(19) United States
(12) Patent Application Publication
(10) Pub. No.: US 2018/0148418 A1
(43) Pub. Date: May 31, 2018

(54) AMIDO-SUBSTITUTED CYCLOHEXANE DERIVATIVES

(71) Applicant: Bayer Pharma Aktiengesellschaft, Berlin (DE)

(72) Inventors: Knut EIS, Berlin (DE); Jens ACKERSTAFF, Düsseldorf (DE); Sarah WAGNER, Düsseldorf (DE); Philipp BUCHGRABER, Berlin (DE); Detlev SÜLZLE, Berlin (DE); Simon HOLTON, Berlin (DE); Eckhard BENDER, Langenfeld (DE); Volkhart LI, Velbert (DE); Ningshu LIU, Berlin (DE); Franziska SIEGEL, Berlin (DE); Philip LIENAU, Berlin (DE); Michaela BAIRLEIN, Wuppertal (DE); Franz VON NUSSBAUM, Lyon (FR); Simon Anthony HERBERT, Berlin (DE); Marcus KOPPITZ, Berlin (DE)

(73) Assignee: Bayer Pharma Aktiengesellschaft, Berlin (DE)

(21) Appl. No.: 15/571,414

(22) PCT Filed: May 2, 2016
(86) PCT No.: PCT/EP2016/059731

§ 371 Date: Nov. 2, 2017

(30) Foreign Application Priority Data
May 5, 2015 (EP) 15166484.4

(51) Int. Cl. C07D 233/90; C07D 495/10; A61P 35/04; C07D 487/10; C07D 487/04; C07D 417/12; C07D 405/14; C07D 405/12; C07D 403/14; C07D 403/06; C07D 401/14; C07D 401/12; C07D 263/34; A61K 45/06; A61K 31/554; A61K 31/5377; A61K 31/519; A61K 31/5025; A61K 31/454; A61K 31/4439; A61K 31/421; A61K 31/4178; A61K 31/4164; A61K 31/4155; A61P 35/00; C07D 491/107

(52) U.S. Cl. CPC C07D 233/90; C07D 491/107; A61P 35/04; C07D 487/10; C07D 487/04; C07D 417/12; C07D 405/14; C07D 405/12; C07D 403/14; C07D 403/06; C07D 401/14; C07D 401/12; C07D 263/34; A61K 45/06; A61K 31/554; A61K 31/5377; A61K 31/519; A61K 31/5025; A61K 31/454; A61K 31/4439; A61K 31/421; A61K 31/4178; A61K 31/4164; A61K 31/4155; A61P 35/00; C07D 495/10

(57) ABSTRACT

The present invention relates to amido-substituted cyclohexane compounds of general formula (I), in which A, R⁴, R⁶, R⁷, R⁸, R⁹, R¹⁰ and R¹¹ are as defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of neoplasms, as a sole agent or in combination with other active ingredient.

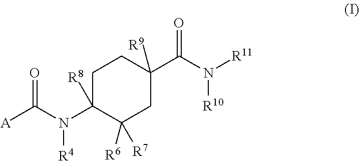

The present invention relates to amido-substituted cyclohexane compounds of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of neoplasms, as a sole agent or in combination with other active ingredients.

### BACKGROUND OF THE INVENTION

Cancer is the leading cause of death in developed countries and the second leading cause of death in developing countries. Deaths from cancer worldwide are projected to continue rising, with an estimated 12 million deaths in 2030. While substantial progress has been made in developing effective therapies, there is a need for additional therapeutic modalities that target cancer and related diseases.

The complexity of cancer disease arises after a selection process for cells with acquired functional capabilities to enhance survival and/or resistance towards apoptosis and a limitless proliferative potential. In addition, bi-direction interaction of cancer cells and stromal cells provides further advantage of cancer cell survival and distant metastasis to the secondary organs and tissues [Liotta L A, Kohn E C. The microenvironment of the tumour-host interface. Nature 2001, 411:375]. Furthermore, cancer stem cells (CSCs) represent the apex in the hierarchical model of tumor genesis, heterogeneity and metastasis. CSCs possess the capacity for unlimited self-renewal, the ability to give rise to progeny cells, and also an innate resistance to cytotoxic therapeutics [Meacham C E and Morrison S J. Tumour heterogeneity and cancer cell plasticity. Nature 2013, 501:328]. Thus, there is need to develop drugs for cancer therapy addressing distinct features of established tumors.

The discovery that Drosophila segment polarity gene Wingless had a common origin with the murine oncogene Int-1 led to intensive studies on Wnt signaling pathway and identification of 19 mammalian Wnts and 10 Wnt receptors [Rijsewijk F, Schuermann M, Wagenaar E, Parren P, Weigel D, Nusse R. The Drosophila homolog of the mouse mammary oncogene int-1 is identical to the segment polarity gene wingless. Cell. 1987, 50: 649]. Wnts are secreted glycoproteins which bind to cell surface receptors to initiate signaling cascades. Wnt signaling cascades have classified into two categories: canonical and non-canonical, differentiated by their dependence on β-catenin. Non-canonical Wnt pathways, such as the planar cell polarity (PCP) and Ca²⁺ pathway, function through β-catenin independent mechanisms. Canonical Wnt signalling is initiated when a Wnt ligand engages co-receptors of the Frizzled (Fzd) and low-density lipoprotein receptor related protein (LRP) families, ultimately leading to β-catenin stabilization, nuclear translocation and activation of target genes [Angers S, Moon R T. Proximal events in Wnt signal transduction. Nat Rev Mol Cell Biol. 2009, 10: 468. Cadigan K M, Liu Y I. Wnt signaling: complexity at the surface. J Cell Sci. 2006, 119: 395. Gordon M D, Nusse R. Wnt signaling: multiple pathways, multiple receptors, and multiple transcription factors. J Biol Chem. 2006, 281: 22429. Huang H, He X. Wnt/beta-catenin signaling: new (and old) players and new insights. Curr Opin Cell Biol. 2008, 20: 119. Polakis P. The many ways of Wnt in cancer. Curr Opin Genet Dev. 2007, 17: 45. Rao T P, Kuhl M. An updated overview on Wnt signaling pathways: a prelude for more. Circ Res. 2010, 106: 1798].

In the absence of Wnt stimulus, β-catenin is held in an inactive state by a multimeric “destruction” complex comprised of adenomatous polyposis coli (APC), Axin, glycogen synthase kinase 3β (GSK3β) and casein kinase 1a (CK1α). APC and Axin function as a scaffold, permitting GSK3β- and CK1α-mediated phosphorylation of critical residues within β-catenin. These phosphorylation events mark β-catenin for ubiquitination recognition by the E3 ubiquitin ligase β-transducin-repeat-containing protein and lead to subsequent proteasomal degradation [He X, Semenov M, Tamai K, Zeng X. LDL receptor-related proteins 5 and 6 in Wnt/beta-catenin signaling: arrows point the way. Development. 2004, 131:1663. Kimelman D, Xu W. beta-catenin destruction complex: insights and questions from a structural perspective. Oncogene 2006, 25: 7482].

In the presence of Wnt stimulus, Axin, GSK3β and Dvl are recruited to the co-receptor complex Fzd and LRP5/6 and lead to disruption of the β-catenin destruction complex. Therefore, β-catenin is stabilized and translocated to the nucleus. Once in the nucleus, β-catenin forms a complex with members of the T-cell factor/lymphoid enhancer factor (TCF/LEF) family of transcription factors, recruiting co-factors such as CBP, p300, TNIK, Bcl9 and Pygopus, and ultimately driving transcription of target genes including c-myc, Oct4, cyclin D, survivin. [Curtin J C and Lorenzi M V. Drug Discovery Approaches to Target Wnt Signaling in Cancer Stem Cells. Oncotarget 2010, 1: 552].

Tankyrases play a key role in the destruction complex by regulating the stability of the rate-limiting AXIN proteins, RNF146 and tankyrase itself. The E3 ubiquitin ligase RNF146 recognizes tankyrase-mediated PARsylation and eartags AXIN, tankyrase and itself for proteasome-mediated degradation. Thus, tankyrases control the protein stability and turnover of key components of the destruction complex, and consequently the cellular levels of β-catenin [Huang S M A, Mishina Y M, Liu S, Cheung A, Stegmeier F, et al. Tankyrase inhibition stabilizes axin and antagonizes Wnt signalling. Nature 2009, 461:614, Zhang Y, Liu S, Mickanin C, Feng Y, Charlat O, et al. RNF146 is a poly(ADP-ribose)-directed E3 ligase that regulates axin degradation and Wnt signalling. Nature Cell Biology 2011, 13:623, 2011].

Aberrant regulation of the Wnt/β-catenin signaling pathway is a common feature across a broad spectrum of human cancers and evolves as a central mechanism in cancer biology. First of all, Wnt overexpression could lead to malignant transformation of mouse mammary tissue [Klaus A, Birchmeier W. Wnt signalling and its impact on development and cancer. Nat Rev Cancer 2008, 8: 387]. Second, tumor genome sequencing discovered the mutations in Wnt/β-catenin pathway components as well as epigenetic mechanisms that altered the expression of genes relevant to Wnt/β-catenin pathway [Ying Y. et al. Epigenetic disruption of the WNT/beta-catenin signaling pathway in human cancers. Epigenetics 2009, 4:307]. Third, Wnt/β-catenin pathway also cooperates with other oncogenic signaling pathways in cancer and regulates tumorigenesis, growth, and metastasis [Klaus A, Birchmeier W. Wnt signalling and its impact on development and cancer. Nat Rev Cancer 8: 387-398, 2008]. In addition, there is an additional role of WNT signaling between tumor and stromal cell interaction leading to tumorigenesis and metastasis [Shahi P, Park D, Pond A C, Seethammagari M, Chiou S-H, Cho K, et al. Activation of Wnt signaling by chemically induced dimerization of LRP5 disrupts cellular homeostasis. PLoS ONE 2012, 7: e30814]. Furthermore, growing body of evidence indicates a critical role of β-catenin in CSCs [Eaves C J, Humphries R K. Acute myeloid leukemia and the Wnt pathway. N Engl J Med. 2010, 362: 2326; Nusse R, Fuerer C, Ching W, Harnish K, Logan C, Zeng A, ten Berge D, Kalani Y. Wnt signaling and stem cell control. Cold Spring Harb Symp Quant Biol. 2008, 73: 59; Reya T, Clevers H. Wnt signalling in stem cells and cancer. Nature 2005, 434: 843]. For example, stem-like colon cells with a high level of β-catenin signaling have a much greater tumorigenic potential than counterpart cells with low β-catenin signaling [Vermeulen L, De Sousa E M F, van der Heijden M, Cameron K, de Jong J H, Borovski T, Tuynman J B, Todaro M, Merz C, Rodermond H, Sprick M R, Kemper K, Richel D J, Stassi G, Medema J P. Wnt activity defines colon cancer stem cells and is regulated by the microenvironment. Nat Cell Biol. 2010, 12: 468]. Finally, activation of Wnt/β-catenin signalling pathway is also one of the major mechanism causing tumor recurrence and drug resistance. All these provide clear rationale to develop therapeutics targeting Wnt/β-catenin signaling pathway for the treatment of cancer.

One of the approaches to inhibit Wnt/β-catenin signaling pathway is to target druggable tankyrases. Tankyrase 1 (TNKS1) and tankyrase 2 (TNKS2) are poly(ADP-ribosyl)ases that are distinguishable from other members of the enzyme family by the structural features of the catalytic domain, and the presence of a sterile a-motif multimerization domain and an ankyrin repeat protein-interaction domain. Inhibition of TNKS blocks PARsylation of AXIN1 and AXIN2 and prevents their proteasomal degradation. As the consequence, TNKS inhibition enhances the activity of the β-catenin destruction complex and suppresses β-catenin nuclear transclocation and the expression of β-catenin target genes.

In addition to its function in Wnt signaling through modulation of β-catenin destruction, tankyrases are also implicated in other cellular functions, including telomere homeostasis, mitotic spindle formation, vesicle transport linked to glucose metabolism, and viral replication. In these processes, tankyrases interact with target proteins, catalyze poly (ADP-ribosyl)ation, and regulate protein interactions and stability. For example, TNKS1 controls telomere homeostasis, which promotes telomeric extension by PARsylating TRF1. TRF1 is then targeted for proteasomal degradation by the E3 ubiquitin ligases F-box only protein 4 and/or RING finger LIM domain-binding protein (RLIM/RNF12), which facilitates telomere maintenance [Donigian J R and de Lange T. The role of the poly(ADP-ribose) polymerase tankyrase 1 in telomere length control by the TRF1 component of the shelterin complex. J Biol Chem 2007, 282:22662]. In addition, telomeric end-capping also requires canonical DNA repair proteins such as DNA-dependent protein kinase (DNAPK). TNKS1 stabilizes the catalytic subunit of DNAPK (DNAPKcs) by PARsylation [Dregalla R C, Zhou J, Idate R R, Battaglia C L, Liber H L, Bailey S M. Regulatory roles of tankyrase 1 at telomeres and in DNA repair: suppression of T-SCE and stabilization of DNA-PKcs. Aging 2010, 2(10):691]. Altered expression of TNKS1 and/or TNKS2, as well as genetic alterations in the tankyrase locus, have been detected in multiple tumors, e.g. fibrosarcoma, ovarian cancer, glioblastoma, pancreatic adenocarcinoma, breast cancer, astrocytoma, lung cancer, gastric cancer, and colon cancer [Lehti L, Chi N-W and Krauss S. Tankyrases as drug targets. FEBS Journal 2013, 280: 3576]. In addition, tankyrases appear to have impact on viral infections. For example, in HSV infection, it was shown that the virus cannot replicate efficiently in cells with depletion of both TNKS1 and TNKS2 [Li Z, Yamauchi Y, Kamakura M, Murayama T, Goshima F, Kimura H, Nishiyama Y, Herpes Simplex Virus Requires Poly(ADP-Ribose) Polymerase Activity for Efficient Replication and Induces Extracellular Signal-Related Kinase-Dependent Phosphorylation and ICPO-Dependent Nuclear Localization of Tankyrase 1. Journal of Virology 2012, 86(1): 492].

Furthermore, a connection between tankyrases and glucose metabolism has been indicated. Thus, DNA polymorphism in a chromosomal region encoding tankyrase/methionine sulfoxide reductase A is robustly associated with early-onset obesity. TNKS1 knockout mice appeared to have reduced fat pads, suggesting a potential connection of TNKS and obesity. TNKS may also play a role in tissue fibrosis.

In summary, tankyrases are promising drug targets in regulating WNT signaling, telomere length (e.g. telomere shortening and DNA damage induced cell death), lung fibrogenesis, myelination and viral infection. The invention presented here describes a novel class of tankyrase inhibitors and their potential clinical utility for the treatment of various diseases, such as cancer, aging, metabolic diseases (e.g. diabetes and obesity), fibrosis (e.g. lung fibrogenesis) and viral infection.

The following list of selected references relates to inhibitors of TNKS1 and/or TNKS2 described in the literature or in patents. However, the chemical structures and compound classes of the inhibitors described in these references are completely different from the chemical structures of the present invention:

Cancer Research 2013, 73 (10): 3132; J Med Chem 2013, 56 (16): 6495; J Med Chem 2013, 56(3): 1341; J Med Chem 2013, 56(17): 7049; J Med Chem 2013, 56(24): 10003; J Med Chem 2013, 56(7): 3012; J Med Chem 2013, 56(20): 7880; J Med Chem 2013, 56(11): 4320; ChemMedChem 2013, 8(12): 1978; ACS Med Chem Lett 2013, 4(12): 1173; ACS Med Chem Lett 2013, 4(12): 1218; Acta Crystallogr Sect F Struct Biol Cryst Commun 2012, 68(Part 2): 115; J Med Chem 2012, 55(3): 1360; WO 2009059994, WO2013164061, WO2014023390, WO 2012076898, WO 2013093508, WO 2013010092, WO 2013189905, WO 2013189865, WO 2013177349, WO 2013012723, WO 2013134079, WO 2013182546, ACS Med Chem Lett, 2014, 6(3): 254.

US2012/004227 (Shionogi & Co.) discloses amido-substituted cyclohexane derivatives as NPY Y5 receptor antagonists.

However, the state of the art described above does not describe the specific substituted cyclohexane compounds of general formula (I) of the present invention as defined herein, bearing:
in its 1-position, a group of structure:

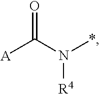

wherein:
indicates the point of attachment of said groups with the rest of the molecule,
A is a five membered nitrogen containing heteroaromatic moiety, selected from

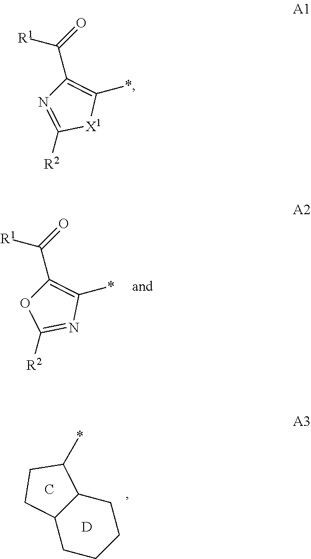

R⁴ is as defined herein;
   and
in its 4-position, a group of structure:

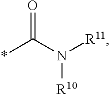

wherein:
indicates the point of attachment of said groups with the rest of the molecule,
R¹⁰ is as defined herein, and
R¹¹ optionally substituted aryl or heteroaryl,
   or a stereoisomer, a tautomer, an N-oxide, a hydrate, a solvate, or a salt thereof, or a mixture of same, as described and defined herein, and as hereinafter referred to as “compounds of the present invention”, or their pharmacological activity.

It has now been found, and this constitutes the basis of the present invention, that said compounds of the present invention have surprising and advantageous properties.

In particular, said compounds of the present invention have surprisingly been found to effectively inhibit TNKS1 and/or TNKS2 and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses mediated by TNKS1 and/or TNKS2 and/or mediated by the Wnt pathway, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof. Compounds of the present invention may additionally show improved selectivity for TNKS1 and/or TNKS2 (e.g. over other PARP (poly(ADP-ribose)-polymerase) enzymes), for the treatment of TNKS1 and/or TNKS2 driven diseases, by reaching sufficient efficacious dose without inducing toxicity driven by, for example, other PARPs inhibition.

### DESCRIPTION OF THE INVENTION

In accordance with a first aspect, the present invention covers compounds of general formula (I):

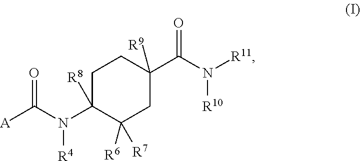

in which:
A represents a group selected from:

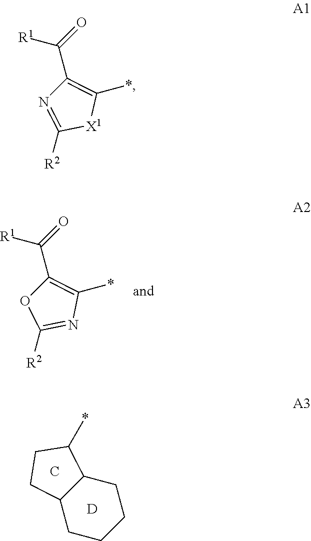

wherein * indicates the point of attachment of said groups with the rest of the molecule, wherein said point of attachment is a carbon atom;

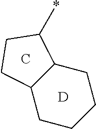

represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by one or two N atoms,
said ring C being optionally substituted with one or two R⁵ groups, and
ring D represents a phenyl group or a 6-membered heteroaryl group which contains one, two or three nitrogen heteroatoms, said ring D being optionally substituted with one, two or three R¹² groups;
X¹ represents NR³ or O,
R¹ represents a group selected from
—OR¹³, and —N(R¹⁴)R¹⁵,
   R² represents a group selected from
hydrogen, C₁-C₃-alkyl, and C₃-C₄-cycloalkyl,
   R³ represents a hydrogen atom,
   R⁴ represents a hydrogen atom,
   R⁵ represents, independently of each other, a group selected from:
halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, and —C(O)OR¹³,
wherein C₁-C₄-alkyl is optionally substituted one, two or three times with a group independently selected from halogen, hydroxy, C₁-C₃-alkoxy, —NH₂, —NH(C₁-C₃-alkyl) and —N(C₁-C₃-alkyl)₂;
   R⁶ represents hydrogen, halogen, hydroxy, C₁-C₃-alkyl or C₁-C₃-alkoxy;
   R⁷ represents hydrogen; or
   R⁶, R⁷ represent, independently of each other, halogen;
   in embodiment a), R⁸, R⁹ and R¹⁰ represent:
   R⁸ represents hydrogen, or C₁-C₃-alkyl,
   R⁹ represents hydrogen, halogen, C₁-C₃-alkoxy, or C₁-C₃-alkyl optionally substituted with one, two or three groups independently selected from hydroxy, halogen and C₃-C₄-cycloalkyl; or
   R⁸ and R⁹ together represent a group:

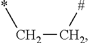

wherein * indicates the point of attachment of said group to the rest of the molecule at R⁸; and
and ^{#} indicates the point of attachment of said group to the rest of the molecule at R⁹; and
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-(C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-; or
in embodiment b), R⁸, R⁹ and R¹⁰ represent:
R⁸ represents hydrogen, or C₁-C₃-alkyl, and
R⁹ and R¹⁰ together represent a group selected from:

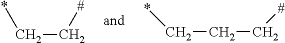

wherein said groups are optionally substituted with one or two groups, which are independently of each other selected from: halogen, C₁-C₃-alkyl and C₁-C₃-alkoxy, hydroxy, C₁-C₃-haloalkyl, C₁-C₃-hydroxyalkyl
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
   wherein aryl and heteroaryl groups are optionally substituted with one, two, three or four groups, which are independently of each other selected from:
   C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
      R¹² represents, independently of each other, halogen, hydroxy, C₁-C₆-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
      wherein C₁-C₆-alkyl is optionally substituted one, two or three times with a substituent independently selected from halogen, hydroxy, C₁-C₃-alkoxy, C₁-C₃-haloalkoxy, and —N(R¹⁸)R¹⁹;
      whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl;
      R¹³ represents a group selected from:
C₁-C₆-alkyl, C₃-C₆-cycloalkyl, C₂-C₆-hydroxyalkyl-, and (C₁-C₃-alkoxy)-(C₂-C₆-alkyl)-,
   R¹⁴ and R¹⁵ are independently of each other selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, —NH(C₁-C₃-alkyl), —N(C₁-C₃-alkyl)₂, hydroxy, a halogen atom, and cyano,
   and,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano, whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
      said azetidine and pyrrolidine being optionally substituted one time with C₁-C₃-alkyl or C₁-C₃-haloalkyl,
      or,
      R¹⁴ and R¹⁵ together with the nitrogen atom to which they are attached form a group selected from:

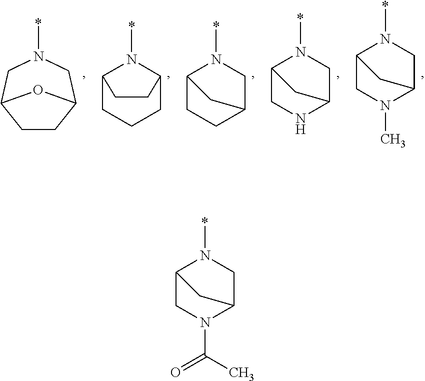

wherein * indicates the point of attachment of said group with the rest of the molecule,
R¹⁶ represents, independently of each other, hydrogen, or C₁-C₃-alkyl,
R¹⁷ represents hydrogen, C₁-C₆-alkyl, C₁-C₆-hydroxyalkyl, C₃-C₆-cycloalkyl, C₁-C₆-haloalkyl, (C₁-C₃-alkoxy)-(C₁-C₆-alkyl)-, aryl, or heteroaryl,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy,
R¹⁸ and R¹⁹ are, independently of each other, selected from:
   hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, and heteroaryl,
      wherein 4- to 6-membered heterocycloalkyl groups are optionally substituted with one two, three or four substituents, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
      and,
      wherein aryl and heteroaryl groups are optionally substituted with one or two substituents, which are independently of each other selected from:
      C₁-C₃-alkyl, halogen, cyano, -
         or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group,
      said azetidine and pyrrolidine being optionally substituted one time with C₁-C₃-alkyl or C₁-C₃-haloalkyl;
      R²⁰ represents, independently of each other, a group selected from:
   hydrogen, C₁-C₃-alkyl, C₁-C₃-haloalkyl, and C₃-C₄-cycloalkyl,
      R²¹ represents hydrogen, cyano, (C₁-C₃-alkyl)-C(═O)—, or (C₁-C₃-haloalkyl)-C(═O)—,
      R²² represents C₁-C₄-alkyl, or C₃-C₄-cycloalkyl,
      or a stereoisomer, a tautomer, an N-oxide, a hydrate, a solvate, or a salt thereof, or a mixture of same.

### Definitions

Constituents which are optionally substituted as stated herein, may be substituted, unless otherwise noted, one or more times, independently from one another at any possible position. When any variable occurs more than one time in any constituent, each definition is independent.

When any variable occurs more than one time in any compound of general formula (I) as described herein, each definition is independent. For example, when R¹³, R¹⁴, R¹⁵, R¹⁶, R¹⁷, R¹⁸, R¹⁹, R²⁰, R²¹, R²², R²³, R²⁴ and/or R²⁵ occur more than one time in any compound of formula (I) each definition of R¹³, R¹⁴, R¹⁵, R¹⁶, R¹⁷, R¹⁸, R¹⁹, R²⁰, R²¹, R²², R²³, R²⁴ and R²⁵ is independent.

A hyphen at the beginning or at the end of the constituent marks the point of attachment to the rest of the molecule. Should a ring be substituted the substitutent could be at any suitable position of the ring, also on a ring nitrogen atom if suitable.

The terms as mentioned in the present text have preferably the following meanings:

The term “comprising” when used in the specification includes “consisting of”.

If it is referred to “as mentioned above” or “mentioned above” within the description it is referred to any of the disclosures made within the specification in any of the preceding pages.

If it is referred to “as mentioned herein”, “as described herein”, “as defined herein”, “as provided herein” or “as stated herein” within the description it is referred to any of the disclosures made within the specification in any of the preceding or subsequent pages.

The term “halogen”, “halogen atom”, “halo-” or “Hal-” is to be understood as meaning a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group, more particularly 1 or 2 carbon atoms (“C₁-C₂-alkyl”), e.g. a methyl, ethyl group, even more particularly 1 carbon atom (“C₁-alkyl”), a methyl group.

The term “C₁-C₆-hydroxyalkyl” is to be understood as meaning a linear or branched, saturated, monovalent hydrocarbon group in which the term “C₁-C₆-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-haloalkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said C₁-C₆-haloalkyl group is, for example, —CF₃, —CHF₂, —CH₂F, —CF₂CF₃, —CH₂CH₂F, —CH₂CHF₂, —CH₂CF₃, or —CH₂CH₂CF₃.

The term “C₁-C₆-alkoxy” is to be understood as preferably meaning a linear or branched, saturated, monovalent, hydrocarbon group of formula —O-alkyl having 1, 2, 3, 4, 5, or 6 carbon atoms, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “C₁-C₆-haloalkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said C₁-C₆-haloalkoxy group is, for example, —OCF₃, —OCHF₂, —OCH₂F, —OCF₂CF₃, or —OCH₂CF₃.

The term “C₃-C₆-cycloalkyl” is to be understood as meaning a saturated, monovalent, monocyclic hydrocarbon ring which contains 3, 4, 5 or 6 carbon atoms (“C₃-C₆-cycloalkyl”). Said C₃-C₆-cycloalkyl group is for example, a monocyclic hydrocarbon ring, e.g. a cyclopropyl, cyclobutyl, cyclopentyl or cyclohexyl ring.

The term “C₃-C₆-cycloalkoxy” is to be understood as preferably meaning a saturated, monovalent, hydrocarbon ring which contains 3, 4, 5 or 6 carbon atoms of formula —O— cycloalkyl, in which the term “cycloalkyl” is defined supra, e.g. a cyclopropyloxy, cyclobutyloxy, cyclopentyloxy or cyclohexyloxy.

The terms “4- to 7-membered heterocycloalkyl” and “4- to 6-membered heterocycloalkyl”, are to be understood as meaning a saturated, monovalent, monocyclic hydrocarbon ring with 4 to 7 or, respectively, 4 to 6 ring atoms in total, and which contains a heteroatom-containing group selected from N, NR²⁰, O, S, S(═O) and S(═O)₂, wherein:
one ring carbon atom is optionally replaced by a further heteroatom-containing group selected from NR²⁰, O, S, S(═O) and S(═O)₂, in which R²⁰ represents a hydrogen atom, or a C₁-C₃-alkyl, C₁-C₃-haloalkyl, or a C₃-C₄-cycloalkyl group, and
one additional ring carbon atom is optionally replaced by C(═O);
   it being possible for said heterocycloalkyl group to be attached to the rest of the molecule via any one of the carbon atoms or, if present, the nitrogen atom.

The term “heteroatom-containing group” as defined herein is to be understood as meaning a group containing a heteroatom, such as NR²⁰, S(═O) and S(═O)₂, and/or a heteroatom such as N, O and S, wherein R²⁰ is as defined herein.

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, or a 6-membered ring, such as tetrahydropyranyl, piperidinyl, morpholinyl, dithianyl, thiomorpholinyl, piperazinyl, or N-methylpiperazinyl. Optionally, said heterocycloalkyl can be benzo fused. Particularly, without being limited thereto, 4- to 6-membered heterocycloalkyl can be selected from piperazinyl, tetrahydro-2H-pyranyl, tetrahydrofuranyl, pyrrolidinyl, piperidinyl, morpholinyl, azetidinyl, 2-oxoimidazolidinyl, 2-oxopyrrolidinyl and 1,1-dioxidothiomorpholinyl. More particularly, without being limited thereto, 4- to 6-membered heterocycloalkyl can be selected from piperazin-1-yl, tetrahydro-2H-pyran-4-yl, tetrahydrofuran-3-yl, pyrrolidin-1-yl, pyrrolidin-2-yl, pyrrolidin-3-yl, piperidin-4-yl, piperidin-1-yl, piperidin-2-yl, piperidin-3-yl, morpholin-4-yl, azetidin-1-yl, tetrahydrofuran-2-yl, 2-oxoimidazolidin-1-yl, 2-oxopyrrolidin-1-yl and 1,1-dioxidothiomorpholin-4-yl.

In certain embodiments, R¹⁴ and R¹⁵ and/or R¹⁸ and R¹⁹ together with the nitrogen atom to which they are attached form a 4- to 7-membered heterocycloalkyl group, in which one ring carbon atom is optionally replaced by a further heteroatom-containing group selected from NR²⁰, O, S, S(═O) and S(═O)₂, and one additional ring carbon atom is optionally replaced by C(═O),
said 4- to 7-membered heterocycloalkyl group being optionally substituted with one, two, three or four groups, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
   whereby when two substituents are attached to the same ring carbon atom, together with the carbon atom to which they are attached, can be linked to one another in such a way that they jointly form a cyclobutanyl, cyclopentanyl, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
   said azetidine and pyrrolidine being optionally substituted one time with C₁-C₃-alkyl or C₁-C₃-haloalkyl.

Particularly, without being limited thereto, when two substituents are attached to the same ring carbon atom of said 4- to 7-membered heterocycloalkyl group, together with the carbon atom to which they are attached, can be linked to one another in such a way that they jointly form groups according to the present invention such as the ones represented below:

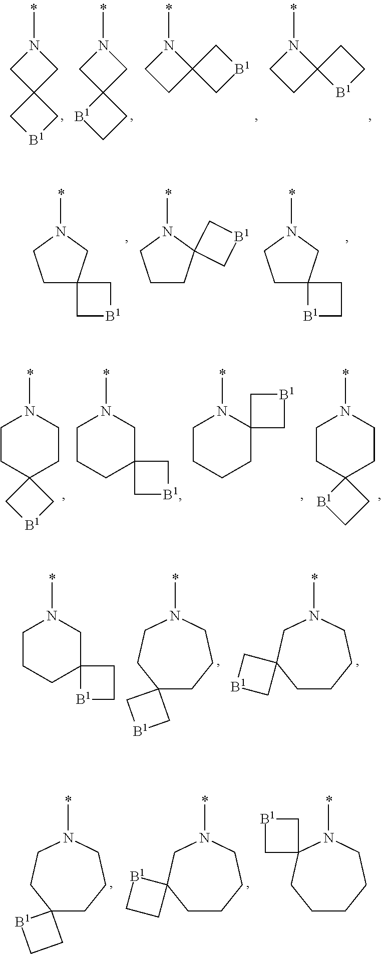

wherein:
* indicates the point of attachment of said group with the rest of the molecule, and
B¹ represents CH₂, —CH₂CH₂—, NH, —CH₂—NH—, N(C₁-C₃-alkyl), —CH₂—N(C₁-C₃-alkyl), N(C₁-C₃-haloalkyl), —CH₂—N(C₁-C₃-haloalkyl)-, O, —CH₂—O—, S, —CH₂—S—, S(O), —CH₂—S(O)—, S(O)₂, or —CH₂—S(O)₂—.

The present invention includes all R¹⁴, R¹⁵ and R¹⁸, R¹⁹ groups described supra.

The term “aryl” is to be understood as meaning a monovalent, aromatic or partially aromatic, mono- or bicyclic hydrocarbon ring having 6, 7, 8, 9 or 10 carbon atoms (a “C₆-C₁₀-aryl” group), particularly a ring having 6 carbon atoms (a “C₆-aryl” group), e.g. a phenyl group; or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group.

The term “heteroaryl” is understood as meaning a monovalent, monocyclic aromatic ring system having 5 or 6 ring atoms (a “5- to 6-membered heteroaryl” group), which contains at least one heteroatom which may be identical or different, said heteroatom being such as oxygen, nitrogen, NH or sulfur. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl etc., or pyridyl, pyridazinyl, pyrimidinyl, pyrazinyl, triazinyl, etc. More particularly, without being limited thereto, heteroaryl can be selected from pyrrolyl, oxazolyl, thiazolyl, imidazolyl, pyrazolyl, triazolyl, or pyridyl, pyridazinyl, pyrimidinyl, pyrazinyl, triazinyl, thienyl, and furanyl. Even more particularly, without being limited thereto, heteroaryl can be selected from oxazolylimidazolyl, pyrazolyl, pyridyl, pyridazinyl, pyrimidinyl and thiazolyl.

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

In general, and unless otherwise mentioned, the heteroarylic radicals include all the possible isomeric forms thereof, e.g. the positional isomers thereof. Thus, for some illustrative non-restricting example, the term pyridinyl includes pyridin-2-yl, pyridin-3-yl and pyridin-4-yl.

In general, and unless otherwise mentioned, aromatic and non-aromatic (hetero)cyclic groups, may optionally be substituted as defined herein. The substituents may be present both when said aromatic and non-aromatic (hetero)cyclic groups exist as a (unitary) constituent, such as, for example, C₃-C₆-cycloalkyl, 4- to 6-membered heterocycloalkyl, aryl and heteroaryl groups, or as part of a constituent composed of more than one part, such as, for example, (C₃-C₆-cycloalkyl)-C₁-C₆-alkyl-, (4- to 6-membered heterocycloalkyl)-(C₂-C₆-alkyl)-,

aryl-(C₁-C₆-alkyl)-, and heteroaryl-(C₁-C₆-alkyl)-, for example. The present invention includes all suitably substituted aromatic and non-aromatic (hetero)cyclic groups both as a (unitary) constituent, or as part of a constituent composed of more than one part. In this context “suitably” is to be understood as meaning chemically possible to be made by methods within the knowledge of a skilled person.

The group

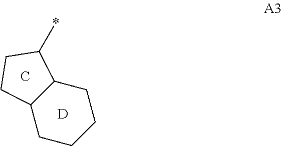

according to the invention represents a bicyclic aromatic ring system, wherein:
indicates the point of attachment of said group with the rest of the molecule, said point of attachment being a carbon atom of ring C;
ring C represents a 5-membered heteroaryl group which contains one heteroatom-containing group selected from N, NH, and N(C₁-C₃-alkyl), in which one or two carbon atoms are optionally further replaced by one or two N atoms, said ring C being optionally substituted with one or two R⁵ groups,
ring D represents a phenyl group or a 6-membered heteroaryl group which contains one, two or three nitrogen atoms, said ring D being optionally substituted with one, two or three R¹² groups,
R⁵ and R¹² are as defined herein.

The total count of nitrogen atoms in ring C includes any nitrogen atoms which are shared with ring D.

Identically, the total count of nitrogen atoms in ring D includes any nitrogen atoms which are shared with ring C.

Particularly, without being limited thereto, said

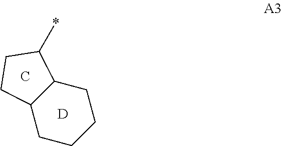

groups according to the present invention can be as represented below:

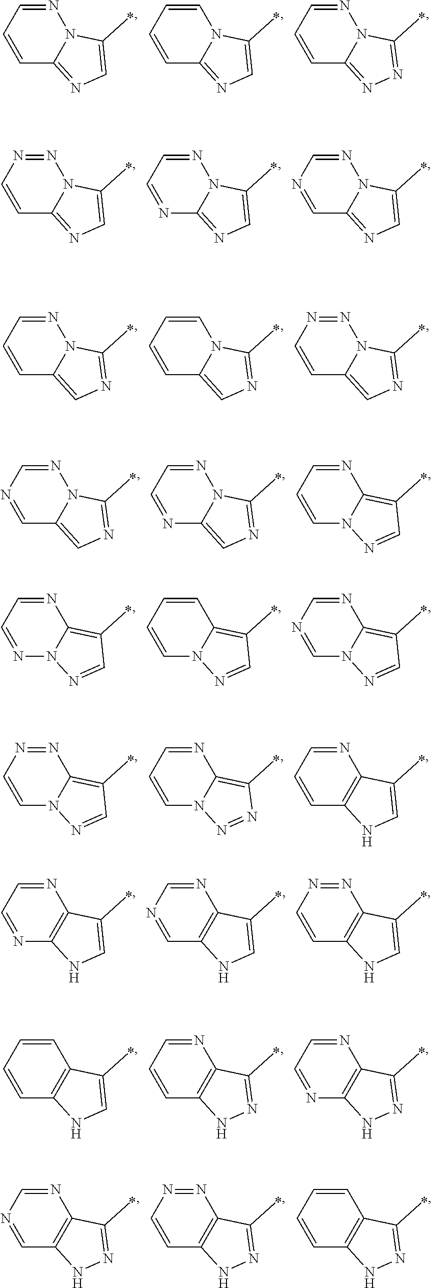

wherein:
indicates the point of attachment of said group with the rest of the molecule, said point of attachment being a carbon atom of ring C;
ring C is optionally substituted with one or two R⁵ groups,
when NH is present in ring C, the nitrogen atom is optionally substituted with C₁-C₃-alkyl,
ring D is optionally substituted with one, two or three R¹² groups, and
R⁵ and R¹² are as defined herein.
   When said

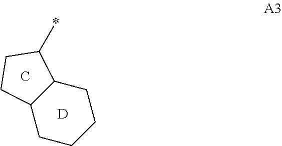

group represents, for example:

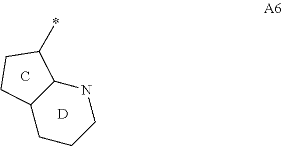

* indicates the point of attachment of said group with the rest of the molecule, said point of attachment being a carbon atom of ring C;
ring C represents a 5-membered heteroaryl group which contains one heteroatom-containing group selected from N, NH, and N(C₁-C₃-alkyl), in which one or two carbon atoms are optionally further replaced by one or two nitrogen atoms, said ring C being optionally substituted with one or two R⁵ groups,
ring D represents a 6-membered heteroaryl group which contains one N atom in the position shown, in which one or two carbon atoms are optionally further replaced by one or two N atoms, said ring D being optionally substituted with one, two or three R¹² groups, and
R⁵ and R¹² are as defined herein.

The present invention includes all

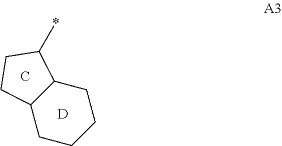

groups described supra, including, but not limited to, A4, A5, A6, A7, A8, A9, A10, A11, A12, A13, A14 and A15.

The term “C₁-C₆”, as used throughout this text, e.g. in the context of the definition of “C₁-C₆-alkyl”, “C₁-C₆-haloalkyl”, “C₁-C₆-hydroxyalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, a used throughout this text, e.g. in the context of the definitions of “C₂-C₆-alkyl”, and “C₂-C₆-hydroxyalkyl” is to be understood as meaning an alkyl group or a hydroxyalkyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, a used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence is preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention optionally contain one or more asymmetric centre, depending upon the location and nature of the various substituents desired. Asymmetric carbon atoms is present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention optionally contain sulphur atoms which are asymmetric, such as an asymmetric sulfoxide, of structure:

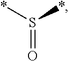

for example,
in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, E- or Z-isomers, or cis or trans, in any ratio. Isolation of a single stereoisomer, e.g. a single enantiomer or a single diastereomer, of a compound of the present invention is achieved by the methods provided herein or by any suitable state of the art method, such as chromatography, especially chiral chromatography, for example.

Particularly, the relative configuration of the amino substituent (NR⁴) relative to the carbonyl group on the central cyclohexane ring can be depicted as follows:

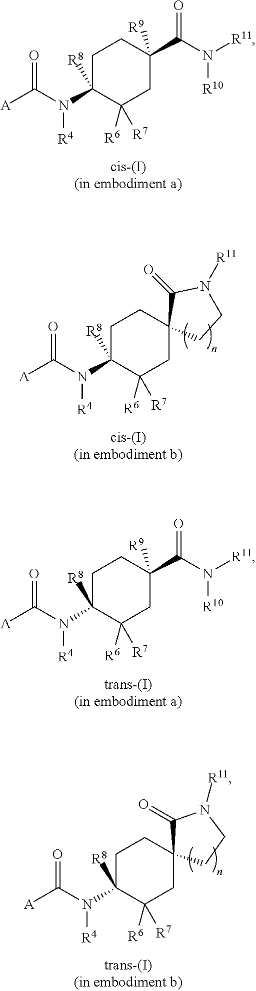

wherein A, R⁴, R⁶, R⁷, R⁸, R⁹, R¹⁰, R¹¹ are as defined herein for the compound of formula (I) and n is 1 or 2.

In respect the cyclohexane ring, the relative configuration of the amino (NR⁴) and carbonyl group substituents on said cyclohexane ring is to be understood as follows:
the term “cis” is to be understood as the relative configuration in which said amino (NR⁴) and carbonyl groups are on the same side of the cyclohexane ring (irrespective of substituents R⁸ and R⁹).
the term “trans” is to be understood as the relative configuration in which said amino (NR⁴) and carbonyl groups are on the opposite side of the cyclohexane ring (irrespective of substituents R⁸ and R⁹).

The present invention includes all cis and trans isomers of the compounds of the present invention as single isomers, or as any mixture of said isomers, in any ratio.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, namely:

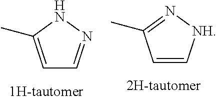

Particularly, when X¹ represents NR³, wherein R³ represents a hydrogen atom, the present invention can exist as one of the below tautomers, or even in a mixture in any amount of the two tautomers, namely:

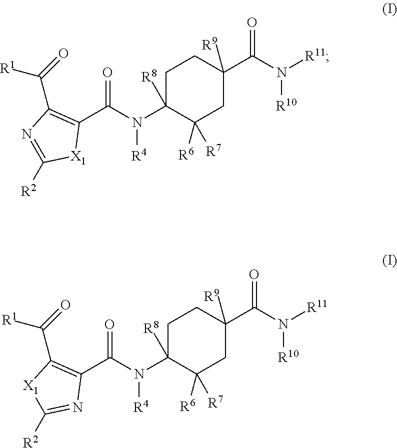

In which R¹, R², R⁴, R⁶, R⁷, R⁸, R⁹, R¹⁰ and R¹¹ are as defined herein.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

In accordance with a second aspect, the present invention covers compounds of general formula (I), supra, in which:

A represents a group selected from:

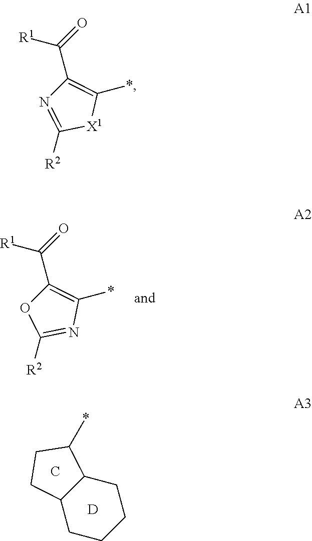

wherein * indicates the point of attachment of said groups with the rest of the molecule, wherein said point of attachment is a carbon atom;

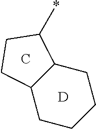

represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by a N atom,
said ring C being optionally substituted with one or two R⁵ groups, and
ring D represents a phenyl group or a 6-membered heteroaryl group which contains one, two or three nitrogen heteroatoms, said ring D being optionally substituted with one, two or three R¹² groups;
X¹ represents NR³ or 0,
R¹ represents a group selected from
—OR¹³, and —N(R¹⁴)R¹⁵,
   R² represents a group selected from
hydrogen, and C₁-C₃-alkyl,
   R³ represents a hydrogen atom,
   R⁴ represents a hydrogen atom,
   R⁵ represents, independently of each other, a group selected from:
halogen, hydroxy, C₁-C₃-alkyl, and —NH₂,
wherein C₁-C₃-alkyl is optionally substituted one, two or three times with a group independently selected from halogen, hydroxy, C₁-C₃-alkoxy, —NH₂, —NH(C₁-C₃-alkyl) and —N(C₁-C₃-alkyl)₂;
   R⁶ represents hydrogen, halogen, hydroxy, C₁-alkyl or C₁-alkoxy;
   R⁷ represents hydrogen; or
   R⁶, R⁷ represent, independently of each other, halogen;
   in embodiment a), R⁸, R⁹ and R¹⁰ represent:
   R⁸ represents hydrogen, or C₁-C₃-alkyl,
   R⁹ represents hydrogen, halogen, C₁-alkoxy or C₁-C₃-alkyl optionally substituted with one, two or three groups independently selected from hydroxy and halogen; or
   R⁸ and R⁹ together represent a group:

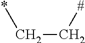

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-; or
in embodiment b), R⁸, R⁹ and R¹⁰ represent:
R⁸ represents hydrogen, or C₁-C₃-alkyl,
R⁹ and R¹⁰ together represent a group selected from:

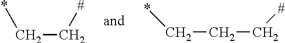

wherein said groups are optionally substituted with one or two groups, which are independently of each other selected from:
halogen, C₁-C₃-alkyl and C₁-C₃-alkoxy, hydroxy, C₁-C₃-haloalkyl, and C₁-C₃-hydroxyalkyl;
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
   wherein aryl and heteroaryl groups are optionally substituted with one, two, three or four groups, which are independently of each other selected from:
   C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
      R¹² represents, independently of each other, halogen, hydroxy, C₁-C₆-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
      wherein C₁-C₆-alkyl is optionally substituted one, two or three times with a substituent independently selected from halogen, hydroxy, C₁-C₃-alkoxy, C₁-C₃-haloalkoxy, —N(R¹⁸)R¹⁹;
      whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl;
      R¹³ represents a group selected from:
C₁-C₆-alkyl, C₃-C₆-cycloalkyl, C₂-C₆-hydroxyalkyl-, and (C₁-C₃-alkoxy)-(C₂-C₆-alkyl)-,
   R¹⁴ and R¹⁵ are independently of each other selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
   and,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, —N(H)₂, N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group,
      said azetidine and pyrrolidine being optionally substituted one time with C₁-C₃-alkyl or C₁-C₃-haloalkyl,
      R¹⁶ represents, independently of each other, hydrogen, or C₁-C₃-alkyl,
      R¹⁷ represents hydrogen, C₁-C₆-alkyl, C₁-C₆-hydroxyalkyl, C₃-C₆-cycloalkyl, C₁-C₆-haloalkyl, (C₁-C₃-alkoxy)-(C₁-C₆-alkyl)-, aryl, or heteroaryl,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy,
      R¹⁸ and R¹⁹ are, independently of each other, selected from:
   hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, and (4- to 6-membered heterocycloalkyl)-(C₁-C₆-alkyl)-,
      wherein 4- to 6-membered heterocycloalkyl groups are optionally substituted with one, two, three or four substituents, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
         or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      R²⁰ represents, independently of each other, a group selected from:
   hydrogen, C₁-C₃-alkyl, C₁-C₃-haloalkyl, and C₃-C₄-cycloalkyl,
      R²¹ represents hydrogen, cyano, (C₁-C₃-alkyl)-C(═O)—, or (C₁-C₃-haloalkyl)-C(═O)—,
      R²² represents C₁-C₄-alkyl, or C₃-C₄-cycloalkyl,
      or a stereoisomer, a tautomer, an N-oxide, a hydrate, a solvate, or a salt thereof, or a mixture of same.

In accordance with a third aspect, the present invention covers compounds of general formula (I), supra, in which:

A represents a group selected from:

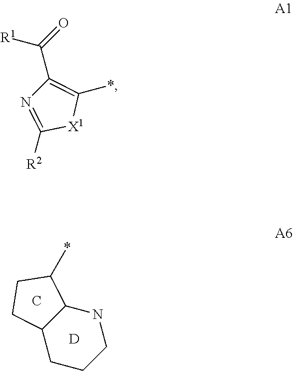

wherein * indicates the point of attachment of said groups with the rest of the molecule, wherein said point of attachment is a carbon atom;

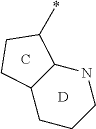

represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by a N atom,
said ring C being optionally substituted with one or two R⁵ groups, and
ring D represents a 6-membered heteroaryl group which contains one, two or three nitrogen heteroatoms, said ring D being optionally substituted with one, two or three R¹² groups;
X¹ represents NR³ or 0,
R¹ represents a group selected from
—OR¹³, and —N(R¹⁴)R¹⁵,
   R² represents a group selected from
hydrogen, and C₁-C₂-alkyl,
   R³ represents a hydrogen atom,
   R⁴ represents a hydrogen atom,
   R⁵ represents, independently of each other, a group selected from:
halogen, hydroxy, C₁-alkyl, and —NH₂,
   R⁶ represents hydrogen, fluorine, hydroxy, C₁-alkyl or C₁-alkoxy;
   R⁷ represents hydrogen; or
   R⁶, R⁷ represent fluorine;
   in embodiment a), R⁸, R⁹ and R¹⁰ represent:
   R⁸ represents hydrogen, or C₁-C₃-alkyl,
   R⁹ represents hydrogen, halogen, or C₁-C₃-alkyl; or
   R⁸ and R⁹ together represent a group:

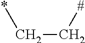

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-(C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-; or
in embodiment b), R⁸, R⁹ and R¹⁰ represent:
R⁸ represents hydrogen, or C₁-C₃-alkyl,
R⁹ and R¹⁰ together represent a group selected from:

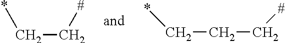

wherein said groups are optionally substituted with one or two groups, which are independently of each other selected from: halogen, C₁-C₃-alkyl and C₁-C₃-alkoxy, hydroxy, C₁-C₃-haloalkyl, C₁-C₃-hydroxyalkyl,
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
   wherein aryl and heteroaryl groups are optionally substituted with one, two, three or four groups, which are independently of each other selected from:
   C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy,
   (C₁-C₆-alkyl)-S—, (C₁-C₆-alkyl)-S(═O)—, (C₁-C₆-alkyl)-S(═O)₂—, —S(═O)(═NR²)R²², —N(R¹⁴)R⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R⁷, —C(═O)OH, —C(═O)OR¹³, and —C(═O)N(R¹⁶)₂,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
      R¹² represents, independently of each other, halogen, hydroxy, C₁-C₆-alkyl, C₃-C₆-cycloalkyl, C₁-C₄-alkoxy, C₁-C₄-haloalkoxy, —N(R¹⁸)R¹⁹, —C(O)R¹³, or —C(O)OR³,
      wherein C₁-C₆-alkyl is optionally substituted one, two or three times with a substituent independently selected from halogen, hydroxy, C₁-C₃-alkoxy, C₁-C₃-haloalkoxy, and —N(R¹⁸)R¹⁹;
      whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl;
      R¹³ represents a group selected from:
C₁-C₃-alkyl, C₃-C₄-cycloalkyl, C₂-C₃-hydroxyalkyl-, and (C₁-alkoxy)-(C₂-C₃-alkyl)-,
   R¹⁴ and R¹⁵ are independently of each other selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl aryl-(C₁-C₆-alkyl)-, and heteroaryl-(C₁-C₆-alkyl)-,
   wherein 4- to 6-membered heterocycloalkyl groups are optionally substituted with one or two substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
   and,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, —N(H)₂, —N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
      said azetidine and pyrrolidine being optionally substituted one time with C₁-C₃-alkyl or C₁-C₃-haloalkyl,
      R¹⁶ represents, independently of each other, hydrogen, or C₁-C₃-alkyl,
      R¹⁷ represents hydrogen, C₁-C₆-alkyl, C₁-C₆-hydroxyalkyl, C₃-C₆-cycloalkyl, C₁-C₆-haloalkyl, (C₁-C₃-alkoxy)-(C₁-C₆-alkyl)-, aryl, or heteroaryl,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy,
      R¹⁸ and R¹⁹ are, independently of each other, selected from:
   hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, and (4- to 6-membered heterocycloalkyl)-(C₁-C₆-alkyl)-,
      wherein 4- to 6-membered heterocycloalkyl groups are optionally substituted with one or two substituents, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
         or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      R²⁰ represents, independently of each other, a group selected from:
   hydrogen, C₁-C₃-alkyl, C₁-C₃-haloalkyl, and C₃-C₄-cycloalkyl,
      R²¹ represents hydrogen, cyano, (C₁-C₃-alkyl)-C(═O)—, or (C₁-C₃-haloalkyl)-C(═O)—,
      R²² represents C₁-C₄-alkyl, or C₃-C₄-cycloalkyl,
      or a stereoisomer, a tautomer, an N-oxide, a hydrate, a solvate, or a salt thereof, or a mixture of same.

In accordance with a fourth aspect, the present invention covers compounds of general formula (I), supra, in which:

A represents a group selected from:

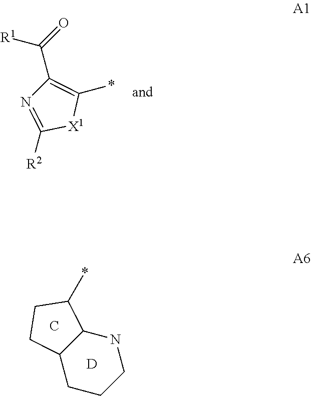

wherein * indicates the point of attachment of said groups with the rest of the molecule, wherein said point of attachment is a carbon atom;

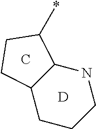

represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R⁵ groups, and
ring D represents a 6-membered heteroaryl group which contains one, two or three nitrogen heteroatoms, said ring being optionally substituted with one, two or three R¹² groups,
X¹ represents NR³ or 0,
R¹ represents a group selected from
—OR¹³, and —N(R¹⁴)R¹⁵,
   R² represents a group selected from
hydrogen, C₁-C₂-alkyl,
   R³ represents a hydrogen atom,
   R⁴ represents a hydrogen atom,
   R⁵ represents, independently of each other, a group selected from:
halogen, hydroxy, C₁-C₂-alkyl, and —NH₂,
wherein C₁-C₂-alkyl is optionally substituted one time with a group selected from hydroxy and C₁-alkoxy;
   R⁶ represents hydrogen, fluorine, C₁-alkyl or C₁-alkoxy;
   R⁷ represents hydrogen; or
   R⁶, R⁷ represent fluorine;
   in embodiment a), R⁸, R⁹ and R¹⁰ represent:
   R⁸ represents hydrogen,
   R⁹ represents hydrogen, fluorine or C₁-alkyl; or
   R⁸ and R⁹ together represent a group:

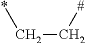

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, or C₁-C₃-haloalkyl; or
in embodiment b), R⁸, R⁹ and R¹⁰ represent:
R⁸ represents hydrogen,
R⁹ and R¹⁰ together represent a group selected from:

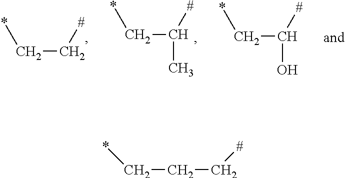

wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
   wherein aryl and heteroaryl groups are optionally substituted with one, two, three or four groups, which are independently of each other selected from:
   C₁-C₄-alkyl, C, —C₃-alkoxy, C₁-C₃-hydroxyalkyl, C₃-C₄-cycloalkyl, C₃-C₄-cycloalkoxy, C₁-C₃-haloalkyl, C₁-C₃-haloalkoxy, halogen, cyano, hydroxy, —C(═O)OR¹³, —N(R¹⁴)R¹⁵, R¹⁴(R¹⁵)N—(C₁-C₃-alkyl)- and R¹⁴(R¹⁵)N—(C₂-C₃-alkoxy)-,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
      R¹² represents, independently of each other, halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, or —C(O)OR¹³,
      wherein C₁-C₄-alkyl is optionally substituted one, two or three times with halogen and optionally substituted one time with a substituent independently selected from hydroxy, C₁-C₃-alkoxy, C₁-C₃-haloalkoxy, —N(R¹⁸)R¹⁹;
      whereby two substituents R¹² when they are in adjacent positions of the ring to which they are attached, can be linked to one another in such a way that they jointly form propane-1,3-diyl;
      R¹³ represents C₁-C₂-alkyl,
      R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, H₂N—(C₂-C₃-alkyl)-, (C₁-C₃-alkyl)N(H)(C₂-C₃-alkyl)-, (C₁-C₃-alkyl)₂N(C₂-C₃-alkyl)-, 4- to 6-membered heterocycloalkyl, (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
      C₁-alkyl, C₁-haloalkyl, C₁-alkoxy, C₁-haloalkoxy, C₃-C₄-cycloalkyl, C₃-C₄-cycloalkoxy, —N(CH₃)₂, —N(H)₂, —N(CH₃)H, hydroxy, and a halogen atom,
      whereby when two substituents are attached to the same ring carbon atom, together with the carbon atom to which they are attached, can be linked to one another in such a way that they jointly form a azetidine, or oxetane;
      said azetidine being optionally substituted one time with C₁-alkyl or C₁-haloalkyl,
      R¹⁸ and R¹⁹ are, independently of each other, selected from:
   hydrogen, C₁-C₄-alkyl, C₃-C₄-cycloalkyl, (C₃-C₄-cycloalkyl)-(C₁-C₃-alkyl)-, C₂-C₄-hydroxyalkyl, (C₁-alkoxy)-(C₂-C₃-alkyl)-, C₁-C₃-haloalkyl, H₂N—(C₂-C₃-alkyl)-, (C₁-alkyl)N(H)(C₂-C₃-alkyl)-, (C₁-alkyl)₂N(C₂-C₃-alkyl)-, HOC(═O)—(C₁-C₃-alkyl)-, R¹³OC(═O)—(C₁-C₃-alkyl)-, 4- to 6-membered heterocycloalkyl, and (4- to 6-membered heterocycloalkyl)-(C₁-C₃-alkyl)-,
      wherein 4- to 6-membered heterocycloalkyl groups are optionally substituted with one or two substituents, which are independently of each other selected from:
      C₁-alkyl, C₁-haloalkyl, C₁-alkoxy, C₁-haloalkoxy, —NH₂, hydroxy, and a halogen atom,
         or,
R¹⁸ and R¹⁹ together with the nitrogen atom to which they are attached form a 5-6-membered heterocycloalkyl group, in which one carbon atom is optionally replaced by a further heteroatom-containing group selected from NR²⁰, 0,
   said 5-6-membered heterocycloalkyl group being optionally substituted with one or two groups, which are independently of each other selected from:
      C₁-alkyl, C₁-haloalkyl, C₁-alkoxy, C₁-haloalkoxy, —NH₂, —N(CH₃)₂, N(CH₃)H, hydroxy, and a halogen atom,
      R²⁰ represents, independently of each other, a group selected from:
   hydrogen, C₁-alkyl, and C₁-C₂-haloalkyl,
      or a stereoisomer, a tautomer, an N-oxide, a hydrate, a solvate, or a salt thereof, or a mixture of same.

In accordance with a fifth aspect, the present invention covers compounds of general formula (I), supra, in which:

A represents a group selected from:

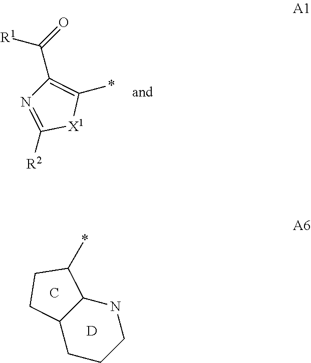

wherein * indicates the point of attachment of said groups with the rest of the molecule, wherein said point of attachment is a carbon atom;

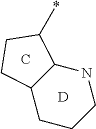

represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R⁵ groups, and
ring D represents a 6-membered heteroaryl group which contains one, two or three nitrogen heteroatoms, said ring being optionally substituted with one, two or three R¹² groups,
X¹ represents NR³ or 0,
R¹ represents a group selected from
—OR¹³, and —N(R¹⁴)R¹⁵,
   R² represents a group selected from
hydrogen, C₁-C₂-alkyl,
   R³ represents a hydrogen atom,
   R⁴ represents a hydrogen atom,
   R⁵ represents, independently of each other, a group selected from:
halogen, hydroxy, C₁-alkyl, and —NH₂,
   R⁶ represents hydrogen, fluorine, C₁-alkyl or C₁-alkoxy;
   R⁷ represents hydrogen; or
   R⁶, R⁷ represent fluorine;
   in embodiment a), R⁸, R⁹ and R¹⁰ represent:
   R⁸ represents hydrogen,
   R⁹ represents hydrogen, or C₁-alkyl; or
   R⁸ and R⁹ together represent a group:

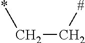

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, or C₁-C₃-haloalkyl; or
in embodiment b), R⁸, R⁹ and R¹⁰ represent:
R⁸ represents hydrogen,
R⁹ and R¹⁰ together represent a group selected from:

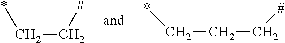

wherein * indicates the point of attachment of said group to the rest of the molecule at R⁹,
and ^{#} indicates the point of attachment of said group to the rest of the molecule at R¹;
R¹¹ represents a group selected from:
aryl, and heteroaryl,
   wherein aryl and heteroaryl groups are optionally substituted with one, two, three or four groups, which are independently of each other selected from:
   C₁-C₄-alkyl, C₁-C₃-alkoxy, C₁-C₃-hydroxyalkyl, C₃-C₄-cycloalkyl, C₃-C₄-cycloalkoxy, C₁-C₅-haloalkyl, C₁-C₅-haloalkoxy, halogen, cyano, hydroxy, R¹⁴(R¹⁵)N—(C₁-C₃-alkyl)-, and R¹⁴(R¹⁵)N—(C₂-C₃-alkoxy)-,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
      R¹² represents, independently of each other, halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, or —C(O)OR¹³,
      wherein C₁-C₄-alkyl is optionally substituted one, two or three times with halogen and optionally substituted one time with a substituent independently selected from hydroxy, C₁-C₃-alkoxy, C₁-C₅-haloalkoxy, —N(R¹⁸)R¹⁹;
      whereby two substituents R¹² when they are in adjacent positions of the ring to which they are attached, can be linked to one another in such a way that they jointly form propane-1,3-diyl;
      R¹³ represents C₁-C₂-alkyl,
      R¹⁴ and R¹⁵ are independently of each other selected from:
hydrogen, C₁-C₃-alkyl, C₃-C₄cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl H₂N—(C₂-C₃-alkyl)-, (C₁-C₅-alkyl)N(H)(C₂-C₃-alkyl)-, (C₁-C₃-alkyl)₂N(C₂-C₃-alkyl)-, 4- to 6-membered heterocycloalkyl, (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
      C₁-alkyl, C₁-haloalkyl, C₁-alkoxy, C₁-haloalkoxy, C₃-C₄-cycloalkyl, C₃-C₄-cycloalkoxy, —N(CH₃)₂, —N(H)₂, —N(CH₃)H, hydroxy, and a halogen atom,
      whereby when two substituents are attached to the same ring carbon atom, together with the carbon atom to which they are attached, can be linked to one another in such a way that they jointly form a azetidine, or oxetane;
      said azetidine being optionally substituted one time with C₁-alkyl or C₁-haloalkyl,
      R¹⁸ and R¹⁹ are, independently of each other, selected from:
   hydrogen, C₁-C₄-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₃-alkyl)-, C₁-C₃-haloalkyl, H₂N—(C₂-C₃-alkyl)-, (C₁-alkyl)N(H)(C₂-C₃-alkyl)-, (C₁-alkyl)₂N(C₂-C₃-alkyl)-, HOC(═O)—(C₁-C₃-alkyl)-, R¹³OC(═O)—(C₁-C₃-alkyl)-, 4- to 6-membered heterocycloalkyl, and (4- to 6-membered heterocycloalkyl)-(C₁-C₃-alkyl)-,
      wherein 4- to 6-membered heterocycloalkyl groups are optionally substituted with one or two substituents, which are independently of each other selected from:
      C₁-alkyl, C₁-haloalkyl, C₁-alkoxy, C₁-haloalkoxy, —NH₂, hydroxy, and a halogen atom,
         or,
R¹⁸ and R¹⁹ together with the nitrogen atom to which they are attached form a 5-6-membered heterocycloalkyl group, in which one carbon atom is optionally replaced by a further heteroatom-containing group selected from NR²⁰, 0,
   said 5-6-membered heterocycloalkyl group being optionally substituted with one or two groups, which are independently of each other selected from:
      C₁-alkyl, C₁-haloalkyl, C₁-alkoxy, C₁-haloalkoxy, —NH₂, —N(CH₃)₂, N(CH₃)H, hydroxy, and a halogen atom,
      R²⁰ represents, independently of each other, a group selected from:
   hydrogen, C₁-alkyl, and C₁-C₂-haloalkyl,
      or a stereoisomer, a tautomer, an N-oxide, a hydrate, a solvate, or a salt thereof, or a mixture of same.

In accordance with a sixth aspect, the present invention covers compounds of general formula (I), supra, in which:

A represents a group selected from:

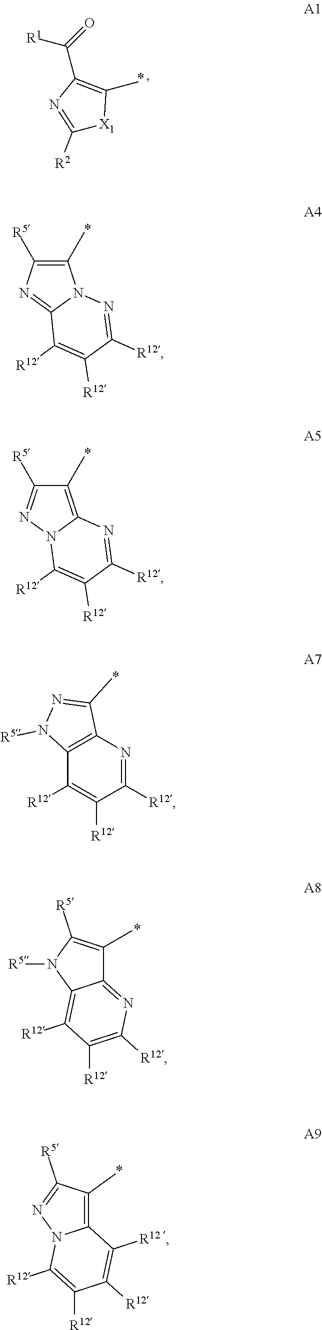

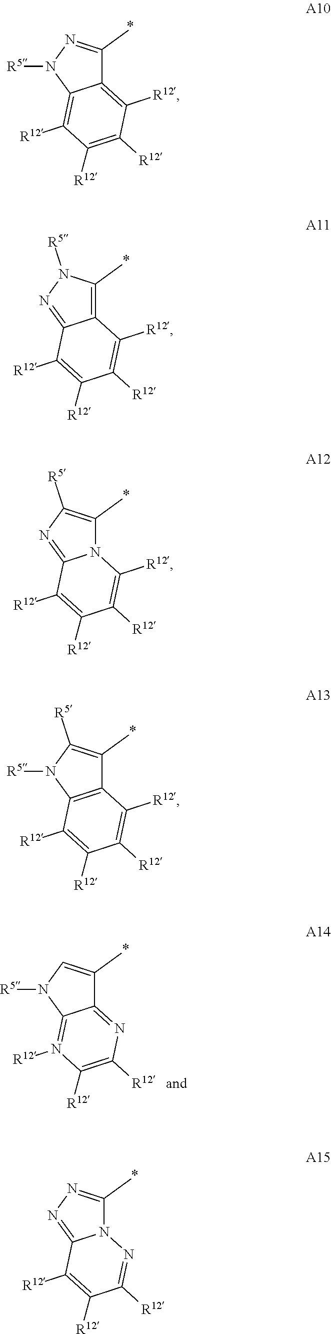

wherein * indicates the point of attachment of said groups with the rest of the molecule,
X¹ represents NR³ or O,
R¹ represents a group selected from:
—OR¹³, and —N(R¹⁴)R¹⁵,
   R² represents hydrogen,
   R³ represents a hydrogen atom,
   R⁴ represents a hydrogen atom,
   R^{5′} represents, independently of each other, a group selected from:
   hydrogen, halogen, hydroxy, C₁-alkyl and —NH₂,
   R^{5′} represents, independently of each other, a group selected from:
   hydrogen, C₁-C₃-alkyl, C₂-hydroxyalkyl and (C₁-alkoxy)-(C₂-alkyl)-,
   R⁶ represents hydrogen, fluorine, C₁-alkyl or C₁-alkoxy;
   R⁷ represents hydrogen; or
   R⁶, R⁷ represent fluorine;
   in embodiment a), R⁸, R⁹ and R¹⁰ represent:
   R⁸ represents hydrogen,
   R⁹ represents hydrogen, fluorine or C₁-alkyl; or
   R⁸ and R⁹ together represent a group:

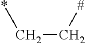

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, or C₂-C₃-hydroxyalkyl; or
in embodiment b), R⁸, R⁹ and R¹⁰ represent:
R⁸ represents hydrogen,
R⁹ and R¹⁰ together represent a group selected from:

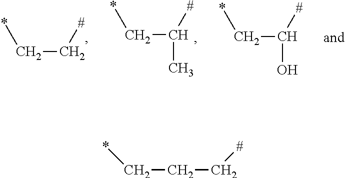

wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
   wherein aryl and heteroaryl groups are optionally substituted with one, two, three or four groups, which are independently of each other selected from:
   C₁-C₄-alkyl, C₁-C₃-alkoxy, C₁-C₃-hydroxyalkyl, C₁-C₃-haloalkyl, C₁-C₃-haloalkoxy, halogen, cyano, —C(═O)OR¹³, and —N(R¹⁴)R¹⁵;
      R^{12′} represents, independently of each other, hydrogen, halogen, hydroxy, C₁-C₄-alkyl, C₃-C₄-cycloalkyl, C₁-alkoxy, —N(R¹⁸)R¹⁹, —C(O)R¹³, or —C(O)OR¹³,
      wherein C₁-C₄-alkyl is optionally substituted one, two or three times, independently of each other, with halogen and optionally substituted one time with a substituent selected from hydroxy, C₁-C₃-alkoxy, —NH₂, —NH(CH₃), —N(CH₃)₂,
      whereby two substituents R^{12′} when they are in adjacent positions of the ring to which they are attached, can be linked to one another in such a way that they jointly form propane-1,3-diyl;
      R¹³ represents C₁-C₂-alkyl,
      R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, 4- to 6-membered heterocycloalkyl and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
   C₁-alkyl, C₁-haloalkyl, C₁-alkoxy, C₁-haloalkoxy, —N(CH₃)₂, hydroxy and a halogen atom,
      whereby when two substituents are attached to the same ring carbon atom, together with the carbon atom to which they are attached, can be linked to one another in such a way that they jointly form a azetidine, thietane 1,1-dioxide, or oxetane;
      said azetidine being optionally substituted one time with C₁-alkyl or C₁-haloalkyl,
      R¹⁸ and R¹⁹ are, independently of each other, selected from:
   hydrogen and C₁-alkyl,
      or,
R¹⁸ and R¹⁹ together with the nitrogen atom to which they are attached form a 5-6-membered heterocycloalkyl group, in which one carbon atom is optionally replaced by a further heteroatom-containing group selected from NR²⁰, and O,
   R²⁰ represents, independently of each other, a group selected from:
   hydrogen, C-alkyl, and C₁-C₂-haloalkyl,
      or a stereoisomer, a tautomer, an N-oxide, a hydrate, a solvate, or a salt thereof, or a mixture of same.

In accordance with a seventh aspect, the present invention covers compounds of general formula (I), supra, in which:

A represents a group selected from:

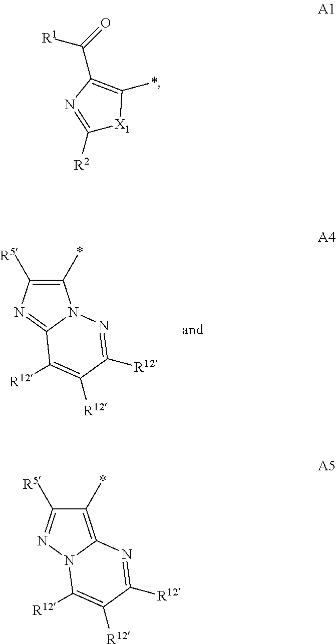

wherein * indicates the point of attachment of said groups with the rest of the molecule,
X¹ represents NR³ or 0,
R¹ represents a group selected from:
—OR¹³, and —N(R¹⁴)R¹⁵,
   R² represents hydrogen,
   R³ represents a hydrogen atom,
   R⁴ represents a hydrogen atom,
   R^{5′} represents, independently of each other, a group selected from:
   hydrogen, halogen, hydroxy, C₁-C₃-alkyl and —NH₂,
   R⁶ represents hydrogen, fluorine, C₁-alkyl or C₁-alkoxy;
   R⁷ represents hydrogen; or
   R⁶, R⁷ represent fluorine;
   in embodiment a), R⁸, R⁹ and R¹⁰ represent:
   R⁸ represents hydrogen,
   R⁹ represents hydrogen, or C₁-alkyl; or
   R⁸ and R⁹ together represent a group:

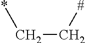

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, or C₂-C₃-hydroxyalkyl; or
in embodiment b), R⁸, R⁹ and R¹⁰ represent:
R⁸ represents hydrogen,
R⁹ and R¹⁰ together represent a group selected from:

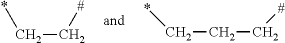

wherein * indicates the point of attachment of said group to the rest of the molecule at R⁹,
and ^{#} indicates the point of attachment of said group to the rest of the molecule at R¹;
R¹¹ represents a group selected from:
aryl, and heteroaryl,
   wherein aryl and heteroaryl groups are optionally substituted with one, two, three or four groups, which are independently of each other selected from:
   C₁-C₄-alkyl, C₁-C₃-alkoxy, C₁-C₃-hydroxyalkyl, C₁-C₃-haloalkyl, halogen and, cyano,
      R^{12′} represents, independently of each other, hydrogen, halogen, hydroxy, C₁-C₄-alkyl, C₃-C₄-cycloalkyl, C₁-alkoxy, —N(R¹⁸)R¹⁹, —C(O)R¹³, or —C(O)OR¹³,
      wherein C₁-C₄-alkyl is optionally substituted one, two or three times, independently of each other, with halogen and optionally substituted one time with a substituent selected from hydroxy, C₁-C₃-alkoxy, —NH₂, —NH(CH₃), —N(CH₃)₂,
      whereby two substituents R^{12′} when they are in adjacent positions of the ring to which they are attached, can be linked to one another in such a way that they jointly form propane-1,3-diyl;
      R¹³ represents C₁-C₂-alkyl,
      R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
   C₁-alkyl, C₁-alkoxy, —N(CH₃)₂, and a halogen atom,
      whereby when two substituents are attached to the same ring carbon atom, together with the carbon atom to which they are attached, can be linked to one another in such a way that they jointly form a azetidine, thietane 1,1-dioxide, or oxetane;
      said azetidine being optionally substituted one time with C₁-alkyl or C₁-haloalkyl,
      R¹⁸ and R¹⁹ are, independently of each other, selected from:
   hydrogen and C₁-alkyl,
      or,
R¹⁸ and R¹⁹ together with the nitrogen atom to which they are attached form a 5-6-membered heterocycloalkyl group, in which one carbon atom is optionally replaced by a further heteroatom-containing group selected from NR²⁰, and O,
   R²⁰ represents, independently of each other, a group selected from:
   hydrogen, C₁-alkyl, and C₁-C₂-haloalkyl,
      or a stereoisomer, a tautomer, an N-oxide, a hydrate, a solvate, or a salt thereof, or a mixture of same.

In accordance with a eighth aspect, the present invention covers a compound of general formula (I), supra, which is selected from the group consisting of:
N⁵-{trans-4-[(2-chloro-4-fluorophenyl)carbamoyl]cyclohexyl}-N⁴-methyl-1H-imidazole-4,5-dicarboxamide
N⁵-{trans-4-[(2-chloro-4-fluorophenyl)carbamoyl]cyclohexyl}-N⁴-[2-(piperidin-1-yl)ethyl]-1H-imidazole-4,5-dicarboxamide
N⁵-{trans-4-[(2-chloro-4,5-difluorophenyl)carbamoyl]cyclohexyl}-N⁴-methyl-1H-imidazole-4,5-dicarboxamide
N⁵-{trans-4-[(2-chloro-4,5-difluorophenyl)carbamoyl]cyclohexyl}-N⁴-[2-(piperidin-1-yl)ethyl]-1H-imidazole-4,5-dicarboxamide
N⁵-{trans-4-[(2-chloro-4,5-dimethoxyphenyl)carbamoyl]cyclohexyl}-N⁴-methyl-1H-imidazole-4,5-dicarboxamide
N⁵-{trans-4-[(2-chloro-4,6-difluorophenyl)carbamoyl]cyclohexyl}-N⁴-methyl-1H-imidazole-4,5-dicarboxamide
N⁵-{trans-4-[(2-bromo-4-fluorophenyl)carbamoyl]cyclohexyl}-N⁴-methyl-1H-imidazole-4,5-dicarboxamide
N⁵-{trans-4-[(4-fluorophenyl)carbamoyl]cyclohexyl}-NM-methyl-1H-imidazole-4,5-dicarboxamide
N⁵-{trans-4-[(2-chloro-4-methylphenyl)carbamoyl]cyclohexyl}-N⁴-methyl-1H-imidazole-4,5-dicarboxamide
N⁵-{trans-4-[(2-chloropyridin-3-yl)carbamoyl]cyclohexyl}-N⁴-methyl-1H-imidazole-4,5-dicarboxamide
N⁵-{trans-4-[(3-chloropyridin-4-yl)carbamoyl]cyclohexyl}-N⁴-methyl-1H-imidazole-4,5-dicarboxamide
N⁵-{trans-4-[(2-chloro-4-fluorophenyl)carbamoyl]cyclohexyl}-N⁴-[2-(4-methylpiperidin-1-yl)ethyl]-1H-imidazole-4,5-dicarboxamide
N⁵-{trans-4-[(2-chloro-4-fluorophenyl)carbamoyl]cyclohexyl}-N⁴-[2-(morpholin-4-yl)ethyl]-1H-imidazole-4,5-dicarboxamide
N-{trans-4-[(2-chloro-4-fluorophenyl)carbamoyl]cyclohexyl}-4-(2-oxa-6-azaspiro[3.3]hept-6-ylcarbonyl)-1H-imidazole-5-carboxamide
N⁵-{trans-4-[(2-chloro-4-fluorophenyl)carbamoyl]cyclohexyl}-N⁴-ethyl-1H-imidazole-4,5-dicarboxamide
N⁵-{trans-4-[(2-chloro-4-fluorophenyl)carbamoyl]cyclohexyl}-N⁴,N⁴-dimethyl-1H-imidazole-4,5-dicarboxamide
N⁵-{trans-4-[(2-chloro-4-fluorophenyl)carbamoyl]cyclohexyl}-N⁴-cyclopropyl-1H-imidazole-4,5-dicarboxamide
N⁵-{trans-4-[(2-chloro-4-fluorophenyl)carbamoyl]cyclohexyl}-N⁴-(2-hydroxyethyl)-1H-imidazole-4,5-dicarboxamide
N⁵-{trans-4-[(2-chloro-4-fluorophenyl)carbamoyl]cyclohexyl}-N⁴-(2-methoxyethyl)-1H-imidazole-4,5-dicarboxamide
N⁵-{trans-4-[(2-chloro-4-fluorophenyl)carbamoyl]cyclohexyl}-N⁴-isopropyl-1H-imidazole-4,5-dicarboxamide
N⁵-{trans-4-[(2-chloro-4-fluorophenyl)carbamoyl]cyclohexyl}-N⁴-(2,2,2-trifluoroethyl)-1H-imidazole-4,5-dicarboxamide
N-{trans-4-[(2-chloro-4-fluorophenyl)carbamoyl]cyclohexyl}-4-[(1,1-dioxido-1-thia-6-azaspiro[3.3]hept-6-yl)carbonyl]-1H-imidazole-5-carboxamide
4-(azetidin-1-ylcarbonyl)-N-{trans-4-[(2-chloro-4-fluorophenyl)carbamoyl]cyclohexyl}-1H-imidazole-5-carboxamide
4 N-{trans-4-[(2-chloro-4-fluorophenyl)carbamoyl]cyclohexyl}-4-[(3-methoxyazetidin-1-yl)carbonyl]-1H-imidazole-5-carboxamide
N⁵-{trans-4-[(2-chloro-4-fluorophenyl)carbamoyl]cyclohexyl}-N⁴-[2-(1-oxa-6-azaspiro[3.3]hept-6-yl)ethyl]-1H-imidazole-4,5-dicarboxamide
N-{trans-4-[(2-chloro-4-fluorophenyl)carbamoyl]cyclohexyl}-4-{[3-(dim ethylamino)azetidin-1-yl]carbonyl}-1H-imidazole-5-carboxamide
N⁵-{trans-4-[(6-bromo-2,3,4-trifluorophenyl)carbamoyl]cyclohexyl}-N⁴-methyl-1H-imidazole-4,5-dicarboxamide
N⁵-{trans-4-[(6-bromo-2,4-difluorophenyl)carbamoyl]cyclohexyl}-N⁴-methyl-1H-imidazole-4,5-dicarboxamide
N⁵-{trans-4-[(2-chloro-4-fluorophenyl)carbamoyl]cyclohexyl}-N⁴-methyl-1,3-oxazole-4,5-dicarboxamide
methyl 4-({trans-4-[(2-chloro-4-fluorophenyl)carbamoyl]cyclohexyl}carbamoyl)-1,3-oxazole-5-carboxylate
N⁴-{(1α,2α,4β)-4-[(2-chloro-4-fluorophenyl)carbamoyl]-2-fluorocyclohexyl}-N⁴-methyl-1H-imidazole-4,5-dicarboxamide
N⁴-{(trans)-4-[(2-chloro-4-fluorophenyl)carbamoyl]-2,2-difluorocyclohexyl}-N⁵-methyl-1H-imidazole-4,5-dicarboxamide
N⁴-{(1R,4R)-4-[(2-chloro-4-fluorophenyl)carbamoyl]-2,2-difluorocyclohexyl}-N⁵-methyl-1H-imidazole-4,5-dicarboxamide
N⁴-{(1 S,4S)-4-[(2-chloro-4-fluorophenyl)carbamoyl]-2,2-difluorocyclohexyl}-N⁴-methyl-1H-imidazole-4,5-dicarboxamide
N⁴-{(1α,2β,4β)-4-[(2-chloro-4-fluorophenyl)carbamoyl]-2-methoxycyclohexyl}-N⁵-methy-1H-imidazole-4,5-dicarboxamide
N⁵-{(1R,2R,4R)-4-[(2-chloro-4-fluorophenyl)carbamoyl]-2-methoxycyclohexyl}-N⁵-methyl-1H-imidazole-4,5-dicarboxamide
N⁵-{(1 S,2S,4S)-4-[(2-chloro-4-fluorophenyl)carbamoyl]-2-methoxycyclohexyl}-MV-methyl-1H-imidazole-4,5-dicarboxamide
N⁴-{4-[(2-chloro-4-fluorophenyl)carbamoyl]-2-methylcyclohexyl}-N⁵-methyl-1H-imidazole-4,5-dicarboxamide
N-{4-[(2-chloro-4-fluorophenyl)carbamoyl]-2-methylcyclohexyl}-5,7-dimethylpyrazolo[1,5-a]pyrimidine-3-carboxamide
N-{trans-4-[(2-chloro-4-fluorophenyl)carbamoyl]cyclohexyl}pyrazolo[1,5-a]pyrimidine-3-carboxamide
N-{trans-4-[(2-chloro-4-fluorophenyl)carbamoyl]cyclohexyl}-5,7-dimethylpyrazolo[1,5-a]pyrimidine-3-carboxamide
N-{trans-4-[(2-chloro-4-fluorophenyl)carbamoyl]cyclohexyl}imidazo[1,2-b]pyridazine-3-carboxamide
6-acetyl-N-{trans-4-[(2-chloro-4-fluorophenyl)carbamoyl]cyclohexyl}-7-methylpyrazolo[1,5-a]pyrimidine-3-carboxamide
6-chloro-N-{trans-4-[(2-chloro-4-fluorophenyl)carbamoyl]cyclohexyl}imidazo[1,2-b]pyridazine-3-carboxamide
N-{trans-4-[(2-chloro-4-fluorophenyl)carbamoyl]cyclohexyl}-6-(morpholin-4-yl)imidazo[1,2-b]pyridazine-3-carboxamide
N-{trans-4-[(2-chloro-4-fluorophenyl)carbamoyl]cyclohexyl}-6-(4-methylpiperazin-1-yl)imidazo[1,2-b]pyridazine-3-carboxamide
N-{trans-4-[(2-chloro-4-fluorophenyl)carbamoyl]cyclohexyl}-6-methoxyimidazo[1,2-b]pyridazine-3-carboxamide
N-{trans-4-[(2-chloro-4-fluorophenyl)carbamoyl]cyclohexyl}-6-(pyrrolidin-1-yl)imidazo[1,2-b]pyridazine-3-carboxamide
N-{trans-4-[(2-chloro-4-fluorophenyl)carbamoyl]cyclohexyl}-6-(dimethylamino)imidazo[1,2-b]pyridazine-3-carboxamide
N-{trans-4-[(2-chloro-4-fluorophenyl)carbamoyl]cyclohexyl}-6-(methylamino)imidazo[1,2-b]pyridazine-3-carboxamide
N-{trans-4-[(2-chloro-4-fluorophenyl)carbamoyl]cyclohexyl}-7-methyl-6-[1-(methylamino)ethyl]pyrazolo[1,5-a]pyrimidine-3-carboxamide formic acid salt
N-{trans-4-[(2-chloro-4-fluorophenyl)carbamoyl]cyclohexyl}-7-methyl-6-[1-(methylamino)ethyl]pyrazolo[1,5-a]pyrimidine-3-carboxamide
N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide formic acid salt
N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide
N-{trans-4-[(2-chloro-4-fluorophenyl)carbamoyl]cyclohexyl}-6-[(1R)-(dimethylamino)ethyl]-7-methylpyrazolo[1,5-a]pyrimidine-3-carboxamide
N-{trans-4-[(2-chloro-4-fluorophenyl)carbamoyl]cyclohexyl}-6-(1-hydroxyethyl)-7-methylpyrazolo[1,5-a]pyrimidine-3-carboxamide
2-amino-N-{trans-4-[(2-chloro-4-fluorophenyl)carbamoyl]cyclohexyl}pyrazolo[1,5-a]pyrimidine-3-carboxamide
7-tert-butyl-N-{trans-4-[(2-chloro-4-fluorophenyl)carbamoyl]cyclohexyl}-5-methylpyrazolo[1,5-a]pyrimidine-3-carboxamide
N-{trans-4-[(2-chloro-4-fluorophenyl)carbamoyl]cyclohexyl}-6-methylpyrazolo[1,5-a]pyrimidine-3-carboxamide
N-{trans-4-[(2-chloro-4-fluorophenyl)carbamoyl]cyclohexyl}-5-cyclopropyl-7-methylpyrazolo[1,5-a]pyrimidine-3-carboxamide
N-{trans-4-[(2-chloro-4-fluoro-5-methylphenyl)carbamoyl]cyclohexyl}-N⁴-methyl-1H-imidazole-4,5-dicarboxamide
N⁵-{trans-4-[(2-chloro-4-fluoro-5-methylphenyl)carbamoyl]cyclohexyl}-N⁴-(tetrahydrofuran-2-ylmethyl)-1H-imidazole-4,5-dicarboxamide
N⁵-{trans-4-[(2-chloro-4,6-difluorophenyl)carbamoyl]cyclohexyl}-N⁴-(1-methoxypropan-2-yl)-1H-imidazole-4,5-dicarboxamide N⁵-{trans-4-[(2-chloro-4,6-difluorophenyl)carbamoyl]cyclohexyl}-N⁴-[(2S)-1-methoxypropan-2-yl]-1H-imidazole-4,5-dicarboxamide
N⁵-{trans-4-[(2-chloro-4,6-difluorophenyl)carbamoyl]cyclohexyl}-N⁴-[(2R)-1-methoxypropan-2-yl]-1H-imidazole-4,5-dicarboxamide
N⁵-{trans-4-[(2-chloro-4,6-difluorophenyl)carbamoyl]cyclohexyl}-N⁴-(2-methoxypropyl)-1H-imidazole-4,5-dicarboxamide
N⁵-{trans-4-[(2-chloro-4,6-difluorophenyl)carbamoyl]cyclohexyl}-N⁴-(tetrahydrofuran-2-ylmethyl)-1H-imidazole-4,5-dicarboxamide
N⁵-{trans-4-[(2-chloro-4,6-difluorophenyl)carbamoyl]cyclohexyl}-N⁴-[(2R)-tetrahydrofuran-2-ylmethyl]-1H-imidazole-4,5-dicarboxamide
N⁵-{trans-4-[(2-chloro-4,6-difluorophenyl)carbamoyl]cyclohexyl}-N⁴-[(2R)-tetrahydrofuran-2-ylmethyl]-1H-imidazole-4,5-dicarboxamide
N-{trans-4-[(2-chloro-4-fluorophenyl)carbamoyl]cyclohexyl}-4-[(3,3-difluoroazetidin-1-yl)carbonyl]-1H-imidazole-5-carboxamide
N⁵-{trans-4-[(2-chloro-4-fluorophenyl)carbamoyl]cyclohexyl}-N⁴-(2-methoxy-2-methylpropyl)-1H-imidazole-4,5-dicarboxamide
N⁵-{trans-4-[(2-chloro-4-fluorophenyl)carbamoyl]cyclohexyl}-N⁴-(2-cyclopropylethyl)-1H-imidazole-4,5-dicarboxamide
N⁵-{trans-4-[(2-chloro-4-fluorophenyl)carbamoyl]cyclohexyl}-N⁴-(2-hydroxy-2-methylpropyl)-1H-imidazole-4,5-dicarboxamide
N⁵-{trans-4-[(2-chloro-4-fluorophenyl)carbamoyl]cyclohexyl}-N⁴-(2-isopropoxyethyl)-1H-imidazole-4,5-dicarboxamide
N⁵-{trans-4-[(2-chloro-4-fluorophenyl)carbamoyl]cyclohexyl}-N⁴-[2-(isopropyl amino)ethyl]-1H-imidazole-4,5-dicarboxamide
N⁵-{trans-4-[(2-chloro-4-fluorophenyl)carbamoyl]cyclohexyl}-N⁴-(tetrahydrofuran-2-ylmethyl)-1H-imidazole-4,5-dicarboxamide
N⁵-{trans-4-[(2-chloro-4-fluorophenyl)carbamoyl]cyclohexyl}-N⁴-[(2 S)-tetrahydrofuran-2-ylmethyl]-1H-imidazole-4,5-dicarboxamide
N⁵-{trans-4-[(2-chloro-4-fluorophenyl)carbamoyl]cyclohexyl}-N⁴-[(2R)-tetrahydrofuran-2-ylmethyl]-1H-imidazole-4,5-dicarboxamide
N⁵-{trans-4-[(2-chloro-4-fluorophenyl)carbamoyl]cyclohexyl}-N⁴-(2-methoxypropyl)-1H-imidazole-4,5-dicarboxamide
N⁵-{trans-4-[(2-chloro-4-fluorophenyl)carbamoyl]cyclohexyl}-N⁴-(1-methoxypropan-2-yl)-1H-imidazole-4,5-dicarboxamide
N⁵-{trans-4-[(2-chloro-4-fluorophenyl)carbamoyl]cyclohexyl}-N⁴-[(2S)-1-methoxypropan-2-yl]-1H-imidazole-4,5-dicarboxamide
N⁵-{trans-4-[(2-chloro-4-fluorophenyl)carbamoyl]cyclohexyl}-N⁴-[(2R)-1-methoxypropan-2-yl]-1H-imidazole-4,5-dicarboxamide
N⁵-{trans-4-[(2-chloro-4,5-difluorophenyl)carbamoyl]cyclohexyl}-N⁴-(2-methoxy-2-methylpropyl)-1H-imidazole-4,5-dicarboxamide
N⁵-{trans-4-[(2-chloro-4,5-difluorophenyl)carbamoyl]cyclohexyl}-N⁴-(tetrahydrofuran-2-ylmethyl)-1H-imidazole-4,5-dicarboxamide
N⁵-{trans-4-[(2-chloro-4,5-difluorophenyl)carbamoyl]cyclohexyl}-N⁴-(1-methoxypropan-2-yl)-1H-imidazole-4,5-dicarboxamide
N⁵-(trans-4-{[2-chloro-5-(morpholin-4-yl)phenyl]carbamoyl}cyclohexyl)-N⁴-methyl-1H-imidazole-4,5-dicarboxamide
N⁵-(trans-4-{[2-chloro-5-(morpholin-4-yl)phenyl]carbamoyl}cyclohexyl)-N⁴-(tetrahydrofuran-2-ylmethyl)-1H-imidazole-4,5-dicarboxamide
N⁵-(trans-4-{[2-chloro-5-(morpholin-4-yl)phenyl]carbamoyl}cyclohexyl)-N⁴-(2-methoxy-2-methylpropyl)-1H-imidazole-4,5-dicarboxamide
N⁵-(trans-4-{[2-chloro-5-(morpholin-4-yl)phenyl]carbamoyl}cyclohexyl)-N⁴-(1-methoxypropan-2-yl)-1H-imidazole-4,5-dicarboxamide
N-(trans-4-{[2-chloro-5-(morpholin-4-yl)phenyl]carbamoyl}cyclohexyl)-4-[(3,3-difluoroazetidin-1-yl)carbonyl]-1H-imidazole-5-carboxamide
N⁵-(trans-4-{[2-chloro-5-(morpholin-4-yl)phenyl]carbamoyl}cyclohexyl)-N⁴-(2-cyclopropylethyl)-1H-imidazole-4,5-dicarboxamide
N⁵-(trans-4-{[2-chloro-5-(morpholin-4-yl)phenyl]carbamoyl}cyclohexyl)-N⁴-(2-hydroxy-2-methylpropyl)-1H-imidazole-4,5-dicarboxamide
N⁵-(trans-4-{[2-chloro-5-(morpholin-4-yl)phenyl]carbamoyl}cyclohexyl)-N⁴-(2,2,2-trifluoroethyl)-1H-imidazole-4,5-dicarboxamide
N⁵-{trans-4-[(4-chloropyridin-3-yl)carbamoyl]cyclohexyl}-N⁴-methyl-1H-imidazole-4,5-dicarboxamide
N⁵-{trans-4-[(4-chloropyridin-3-yl)carbamoyl]cyclohexyl}-N⁴-(tetrahydrofuran-2-ylmethyl)-1H-imidazole-4,5-dicarboxamide
N⁵-{trans-4-[(4-chloropyridin-3-yl)carbamoyl]cyclohexyl}-N⁴-(2-methoxy-2-methyl propyl)-1H-imidazole-4,5-dicarboxamide
N⁵-{trans-4-[(4-chloropyridin-3-yl)carbamoyl]cyclohexyl}-N⁴-(1-methoxypropan-2-yl)-1H-imidazole-4,5-dicarboxamide
N⁵-{trans-4-[(2-chloro-4-fluoro-5-methylphenyl)carbamoyl]cyclohexyl}-N⁴-(2-hydroxy-2-methylpropyl)-1H-imidazole-4,5-dicarboxamide
N⁵-{trans-4-[(2-chloro-4-fluoro-5-methylphenyl)carbamoyl]cyclohexyl}-N⁴-(2-methoxy-2-methylpropyl)-1H-imidazole-4,5-dicarboxamide
N-{trans-4-[(3-chloropyridin-4-yl)carbamoyl]cyclohexyl}-5,7-dimethylpyrazolo[1,5-a]pyrimidine-3-carboxamide
N-{trans-4-[(3-chloropyridin-4-yl)carbamoyl]cyclohexyl}imidazo[1,2-b]pyridazine-3-carboxamide
N-{trans-4-[(3-chloropyridin-4-yl)carbamoyl]cyclohexyl}-6-methylpyrazolo[1,5-a]pyrimidine-3-carboxamide
N-{trans-4-[(3-chloropyridin-4-yl)carbamoyl]cyclohexyl}pyrazolo[1,5-a]pyrimidine-3-carboxamide
ethyl 3-({trans-4-[(2-chloro-4-fluorophenyl)carbamoyl]cyclohexyl}carbamoyl)pyrazolo[1,5-a]pyrimidine-6-carboxylate
N-{trans-4-[(2-chloro-4-fluorophenyl)carbamoyl]cyclohexyl}-5-(morpholin-4-y)pyrazolo[1,5-a]pyrimidine-3-carboxamide
N-{trans-4-[(2-chloro-4-fluorophenyl)carbamoyl]cyclohexyl}-6-(pyrrolidin-1-yl)pyrazolo[1,5-a]pyrimidine-3-carboxamide
N-{trans-4-[(2-chloro-4-fluorophenyl)carbamoyl]cyclohexyl}-6-(4-methylpiperazin-1-yl)pyrazolo[1,5-a]pyrimidine-3-carboxamide
N-{trans-4-[(2-chloro-4-fluorophenyl)carbamoyl]cyclohexyl}-5-(methoxymethyl)-7-methylpyrazolo[1,5-a]pyrimidine-3-carboxamide
N-{trans-4-[(2-chloro-4-fluorophenyl)carbamoyl]cyclohexyl}-7-(methoxymethyl)-5-methylpyrazolo[1,5-a]pyrimidine-3-carboxamide
N-{trans-4-[(2-chloro-4,6-difluorophenyl)carbamoyl]cyclohexyl}-5-(methoxymethyl)-7-methylpyrazolo[1,5-a]pyrimidine-3-carboxamide
N-{trans-4-[(2-chloro-4-fluorophenyl)carbamoyl]cyclohexyl}-5-methyl-7,8-dihydro-6H-cyclopenta[e]pyrazolo[1,5-a]pyrimidine-3-carboxamide
N-{trans-4-[(2-chloro-4-fluorophenyl)carbamoyl]cyclohexyl}-6-[(2-hydroxypropyl)amino]imidazo[1,2-b]pyridazine-3-carboxamide N⁵-{4-[(2-chloro-4-fluorophenyl)carbamoyl]bicyclo[2.2.2]oct-1-yl}-N⁴-methyl-1H-imidazole-4,5-dicarboxamide
N⁴-{4-[(2-chloro-4,6-difluorophenyl)carbamoyl]bicyclo[2.2.2]oct-1-yl}-N⁵-methyl-1H-imidazole-4,5-dicarboxamide
N-{4-[(2-chloro-4-fluorophenyl)carbamoyl]bicyclo[2.2.2]oct-1-yl}pyrazolo[1,5-a]pyrimidine-3-carboxamide
N-{4-[(2-chloro-4-fluoro-5-methyl phenyl)carbamoyl]bicyclo[2.2.2]oct-1-yl}pyrazolo[1,5-a]pyrimidine-3-carboxamide
N-{4-[(2-chloro-4,6-difluorophenyl)carbamoyl]bicyclo[2.2.2]oct-1-yl}pyrazolo[1,5-a]pyrimidine-3-carboxamide
N-{4-[(4-chloropyridin-3-yl)carbamoyl]bicyclo[2.2.2]oct-1-yl}pyrazolo[1,5-a]pyrimidine-3-carboxamide
N-{4-[(2-chloro-4-fluorophenyl)carbamoyl]bicyclo[2.2.2]oct-1-yl}imidazo[1,2-b]pyridazine-3-carboxamide
N-{4-[(2-chloro-4-fluoro-5-methyl phenyl)carbamoyl]bicyclo[2.2.2]oct-1-yl}imidazo[1,2-b]pyridazine-3-carboxamide
N-{4-[(2-chloro-4,6-difluorophenyl)carbamoyl]bicyclo[2.2.2]oct-1-yl}imidazo[1,2-b]pyridazine-3-carboxamide
N-{4-[(4-chloropyridin-3-yl)carbamoyl]bicyclo[2.2.2]oct-1-yl}imidazo[1,2-b]pyridazine-3-carboxamide
N-{4-[(2-chloro-4-fluorophenyl)carbamoyl]bicyclo[2.2.2]oct-1-yl}-5,7-dimethylpyrazolo[1,5-a]pyrimidine-3-carboxamide
N-{4-[(2-chloro-4-fluoro-5-methyl phenyl)carbamoyl]bicyclo[2.2.2]oct-1-yl}-5,7-dimethylpyrazolo[1,5-a]pyrimidine-3-carboxamide
N-{4-[(2-chloro-4,6-difluorophenyl)carbamoyl]bicyclo[2.2.2]oct-1-yl}-5,7-dimethylpyrazolo[1,5-a]pyrimidine-3-carboxamide
N-{4-[(4-chloropyridin-3-yl)carbamoyl]bicyclo[2.2.2]oct-1-yl}-5,7-dimethylpyrazolo[1,5-a]pyrimidine-3-carboxamide
N⁵-{trans-4-[(2-chloro-5-fluorophenyl)carbamoyl]cyclohexyl}-N⁴-(tetrahydrofuran-2-ylmethyl)-1H-imidazole-4,5-dicarboxamide
N-{trans-4-[(2-chloro-4,5-difluorophenyl)carbamoyl]cyclohexyl}pyrazolo[1,5-a]pyrimidine-3-carboxamide
N-{trans-4-[(2-chloro-4,6-difluorophenyl)carbamoyl]cyclohexyl}pyrazolo[1,5-a]pyrimidine-3-carboxamide
N-{trans-4-[(2-chloro-4,5-difluorophenyl)carbamoyl]cyclohexyl}imidazo[1,2-b]pyridazine-3-carboxamide
N-{trans-4-[(2-chloro-4,6-difluorophenyl)carbamoyl]cyclohexyl}imidazo[1,2-b]pyridazine-3-carboxamide
N-{trans-4-[(2-chloro-4,5-difluorophenyl)carbamoyl]cyclohexyl}-5,7-dimethylpyrazolo[1,5-a]pyrimidine-3-carboxamide
N-{trans-4-[(2-chloro-4,6-difluorophenyl)carbamoyl]cyclohexyl}-5,7-dimethylpyrazolo[1,5-a]pyrimidine-3-carboxamide
N-[(trans)-2-(2-chloro-4-fluorophenyl)-1-oxo-2-azaspiro[4.5]dec-8-yl]pyrazolo[1,5-a]pyrimidine-3-carboxamide
N-[(trans)-2-(2-chloro-4-fluorophenyl)-1-oxo-2-azaspiro[4.5]dec-8-yl]-6-methylpyrazolo[1,5-a]pyrimidine-3-carboxamide
N-[(trans)-2-(2-chloro-4-fluorophenyl)-1-oxo-2-azaspiro[4.5]dec-8-yl]imidazo[1,2-b]pyridazine-3-carboxamide
N-[(trans)-2-(2-chloro-4-fluorophenyl)-1-oxo-2-azaspiro[4.5]dec-8-yl]-5,7-dimethylpyrazolo[1,5-a]pyrimidine-3-carboxamide
N⁵-[(trans)-2-(2-chloro-4-fluorophenyl)-1-oxo-2-azaspiro[4.5]dec-8-yl]-N⁵-methyl-1H-imidazole-4,5-dicarboxamide
N⁴-[(trans)-2-(2-chloro-4-fluorophenyl)-1-oxo-2-azaspiro[4.5]dec-8-yl]-N⁵-ethyl-1H-imidazole-4,5-dicarboxamide
N⁵-(trans-4-{[2-chloro-5-(trifluoromethyl)phenyl]carbamoyl}cyclohexyl)-N-methyl-1H-imidazole-4,5-dicarboxamide
N⁵-(trans-4-{[2-chloro-5-(trifluoromethyl)phenyl]carbamoyl}cyclohexyl)-N-(1-methoxypropan-2-yl)-1H-imidazole-4,5-dicarboxamide
N⁵-(trans-4-{[2-chloro-5-(trifluoromethyl)phenyl]carbamoyl}cyclohexyl)-N⁴-(2-methoxy-2-methylpropyl)-1H-imidazole-4,5-dicarboxamide
N⁵-(trans-4-{[2-chloro-5-(trifluoromethyl)phenyl]carbamoyl}cyclohexyl)-N⁴-(2-hydroxy-2-methylpropyl)-1H-imidazole-4,5-dicarboxamide
N⁵-(trans-4-{[2-chloro-5-(trifluoromethyl)phenyl]carbamoyl}cyclohexyl)-N⁴-(tetrahydrofuran-2-ylmethyl)-1H-imidazole-4,5-dicarboxamide
N⁵-{trans-4-[(5-chloropyrimidin-4-yl)carbamoyl]cyclohexyl}-N⁴-methyl-1H-imidazole-4,5-dicarboxamide
N-[(cis)-2-(2-chloro-4-fluorophenyl)-1-oxo-2-azaspiro[4.5]dec-8-yl]pyrazolo[1,5-a]pyrimidine-3-carboxamide
N-(trans-4-{[4-chloro-6-(trifluoromethyl)pyridin-3-yl]carbamoyl}cyclohexyl)pyrazolo[1,5-a]pyrimidine-3-carboxamide
N-{trans-4-[(2-chloro-4-fluoro-5-methylphenyl)carbamoyl]cyclohexyl}pyrazolo[1,5-a]pyrimidine-3-carboxamide
N-{trans-4-[(2-chloro-4-fluoro-6-methylphenyl)carbamoyl]cyclohexyl}pyrazolo[1,5-a]pyrimidine-3-carboxamide
N-{trans-4-[(2-chloro-6-cyano-4-fluorophenyl)carbamoyl]cyclohexyl}pyrazolo[1,5-a]pyrimidine-3-carboxamide
N-{trans-4-[(5-chloro-2-methylpyridin-4-yl)carbamoyl]cyclohexyl}pyrazolo[1,5-a]pyrimidine-3-carboxamide
N-{trans-4-[(2-chloro-4-fluorophenyl)(ethyl)carbamoyl]cyclohexyl}pyrazolo[1,5-a]pyrimidine-3-carboxamide
N-{trans-4-[(2-chloro-4-fluorophenyl)(methyl)carbamoyl]cyclohexyl}pyrazolo[1,5-a]pyrimidine-3-carboxamide
N-{trans-4-[methyl(phenyl)carbamoyl]cyclohexyl}pyrazolo[1,5-a]pyrimidine-3-carboxamide
N⁵-{trans-4-[(2-chloro-4-fluorophenyl)(2-hydroxyethyl)carbamoyl]cyclohexyl}-N⁴-methyl-1H-imidazole-4,5-dicarboxamide
MV-{trans-4-[(2-chlorophenyl)(ethyl)carbamoyl]cyclohexyl}-N⁴-methyl-1H-imidazole-4,5-dicarboxamide
N⁵-[(trans)-2-(2-chloro-4-fluorophenyl)-1-oxo-2-azaspiro[5.5]undec-9-yl]-N⁴-methyl-1H-imidazole-4,5-dicarboxamide)
N⁵-[(trans)-2-(2-chloro-4-fluorophenyl)-1-oxo-2-azaspiro[4.5]dec-8-yl]-N⁴-[2-(4-methylpiperidin-1-yl)ethyl]-1H-imidazole-4,5-dicarboxamide
N⁵-[(trans)-2-(2-chloro-4-fluorophenyl)-1-oxo-2-azaspiro[4.5]dec-8-yl]-N⁴-[2-(piperidin-1-yl)ethyl]-1H-imidazole-4,5-dicarboxamide
N⁵-[(trans)-2-(2-chloro-4-fluorophenyl)-1-oxo-2-azaspiro[4.5]dec-8-yl]-N⁴-isopropyl-1H-imidazole-4,5-dicarboxamide
N⁵-[(trans)-2-(2-chloro-4-fluorophenyl)-1-oxo-2-azaspiro[4.5]dec-8-yl]-N⁴-(2-methoxyethyl)-1H-imidazole-4,5-dicarboxamide
N⁵-[(trans)-2-(2-chloro-4-fluorophenyl)-1-oxo-2-azaspiro[4.5]dec-8-yl]-N⁴-(1-methoxypropan-2-yl)-1H-imidazole-4,5-dicarboxamide
N⁵-[(trans)-2-(2-chloro-4-fluorophenyl)-1-oxo-2-azaspiro[4.5]dec-8-yl]-N⁴-cyclopropyl-1H-imidazole-4,5-dicarboxamide
N-[(trans)-2-(2-chloro-4-fluorophenyl)-1-oxo-2-azaspiro[4.5]dec-8-yl]-4-[(3-fluoroazetidin-1-yl)carbonyl]-1H-imidazole-5-carboxamide
N-[(trans)-2-(2-chloro-4-fluorophenyl)-1-oxo-2-azaspiro[4.5]dec-8-yl]-4-{[4-(2,2,2-trifluoroethyl)piperazin-1-yl]carbonyl}-1H-imidazole-5-carboxamide
N-[2-(2-chloro-4,5-difluorophenyl)-1-oxo-2-azaspiro[4.5]dec-8-yl]pyrazolo[1,5-a]pyrimidine-3-carboxamide (isomer 1)
N-{trans-4-[(4-chloropyridin-3-yl)carbamoyl]cyclohexyl}-5,7-dimethylpyrazolo[1,5-a]pyrimidine-3-carboxamide
N⁵-(trans-4-{[2-chloro-5-(2-hydroxypropan-2-yl)phenyl]carbamoyl}cyclohexyl)-N⁴-methyl-1H-imidazole-4,5-dicarboxamide
N⁴-methyl-N⁵-{trans-4-[methyl(phenyl)carbamoyl]cyclohexyl}-1H-imidazole-4,5-dicarboxamide
N⁵-{trans-4-[(4-fluorophenyl)(methyl)carbamoyl]cyclohexyl}-N⁴-methyl-1H-imidazole-4,5-dicarboxamide
N⁵-{trans-4-[(2-chloro-4-fluorophenyl)(methyl)carbamoyl]cyclohexyl}-N⁴-methyl-1H-imidazole-4,5-dicarboxamide
N⁵-{trans-4-[(2-chloro-6-cyano-4-fluorophenyl)carbamoyl]cyclohexyl}-N⁴-methyl-1H-imidazole-4,5-dicarboxamide
N⁵-{trans-4-[(4-chloro-3-methyl-1,2-thiazol-5-yl)carbamoyl]cyclohexyl}-N⁴-methyl-1H-imidazole-4,5-dicarboxamide
N-{trans-4-[(2-chloro-4-fluoro-5-methylphenyl)(methyl)carbamoyl]cyclohexyl}pyrazolo[1,5-a]pyrimidine-3-carboxamide
N-{trans-4-[(2-chloro-4-fluorophenyl)(cyclopropylmethyl)carbamoyl]cyclohexyl}pyrazolo[1,5-a]pyrimidine-3-carboxamide
N-{trans-4-[methyl(2-methylphenyl)carbamoyl]cyclohexyl}pyrazolo[1,5-a]pyrimidine-3-carboxamide
N-{trans-4-[(2-chloro-4-fluorophenyl)carbamoyl]-4-methylcyclohexyl}pyrazolo[1,5-a]pyrimidine-3-carboxamide
N-{cis-4-[(2-chloro-4-fluorophenyl)carbamoyl]-4-methylcyclohexyl}pyrazolo[1,5-a]pyrimidine-3-carboxamide
N⁴-{cis-4-[(2-chloro-4-fluorophenyl)carbamoyl]-4-methylcyclohexyl}-N⁵-methyl-1H-imidazole-4,5-dicarboxamide
N⁵-[(cis)-2-(2-chloro-4-fluorophenyl)-1-oxo-2-azaspiro[4.5]dec-8-yl]-N⁴-methyl-1H-imidazole-4,5-dicarboxamide
N-[(trans)-2-(2-chloro-4-fluorophenyl)-1-oxo-2-azaspiro[4.5]dec-8-yl]-4-[(6-methyl-1,1-dioxido-1,4-thiazepan-4-yl)carbonyl]-1H-imidazole-5-carboxamide
N-[(trans)-2-(2-chloro-4-fluorophenyl)-1-oxo-2-azaspiro[4.5]dec-8-yl]-4-[(2-methyl-2,6-diazaspiro[3.4]oct-6-yl)carbonyl]-1H-imidazole-5-carboxamide, and
N-[(trans)-2-(2-chloro-4-fluorophenyl)-1-oxo-2-azaspiro[4.5]dec-8-yl]-4-[(6-methyl-2,6-diazaspiro[3.3]hept-2-yl)carbonyl]-1H-imidazole-5-carboxamide,
N⁵-[(trans)-2-(2-chloro-4-fluorophenyl)-1-oxo-2-azaspiro[4.5]dec-8-yl]-1H-imidazole-4,5-dicarboxamide
N⁵-[(trans)-2-(2-chloro-4-fluorophenyl)-1-oxo-2-azaspiro[4.5]dec-8-yl]-N⁴-(1-methylazetidin-3-yl)-1H-imidazole-4,5-dicarboxamide
N-[(trans)-2-(2-chloro-4-fluorophenyl)-1-oxo-2-azaspiro[4.5]dec-8-yl]-4-[(3-methoxyazetidin-1-yl)carbonyl]-1H-imidazole-5-carboxamide
N-[(trans)-2-(2-chloro-4-fluorophenyl)-1-oxo-2-azaspiro[4.5]dec-8-yl]-4-[(3,3-difluoroazetidin-1-yl)carbonyl]-1H-imidazole-5-carboxamide
N-[(trans)-2-(2-chloro-4-fluorophenyl)-1-oxo-2-azaspiro[4.5]dec-8-yl]-4-{[3-(difluoromethyl)azetidin-1-yl]carbonyl}-1H-imidazole-5-carboxamide
N-[(trans)-2-(2-chloro-4-fluorophenyl)-1-oxo-2-azaspiro[4.5]dec-8-yl]-4-[(3-hydroxyazetidin-1-yl)carbonyl]-1H-imidazole-5-carboxamide
N-[(trans)-2-(2-chloro-4-fluorophenyl)-1-oxo-2-azaspiro[4.5]dec-8-yl]-4-{[3-(difluoromethoxy)azetidin-1-yl]carbonyl}-1H-imidazole-5-carboxamide
N-[(trans)-2-(2-chloro-4-fluorophenyl)-1-oxo-2-azaspiro[4.5]dec-8-yl]-4-(2-oxa-6-azaspiro[3.3]hept-6-ylcarbonyl)-1H-imidazole-5-carboxamide
N⁴-[(trans)-2-(2-chloro-4-fluorophenyl)-1-oxo-2-azaspiro[4.5]dec-8-yl]-N⁵-(2,2,2-trifluoroethyl)-1H-imidazole-4,5-dicarboxamide
N⁵-[(trans)-2-(2-chloro-4,5-difluorophenyl)-1-oxo-2-azaspiro[4.5]dec-8-yl]-N⁴-methyl-1H-imidazole-4,5-dicarboxamide
N⁵-[(trans)-2-(2-chloro-4,5-difluorophenyl)-1-oxo-2-azaspiro[4.5]dec-8-yl]-N⁴-ethyl-1H-imidazole-4,5-dicarboxamide
N⁵-[(trans)-2-(2-chloro-4,5-difluorophenyl)-1-oxo-2-azaspiro[4.5]dec-8-yl]-N⁴-(propan-2-yl)-1H-imidazole-4,5-dicarboxamide
N⁵-[(trans)-2-(2-chloro-4,5-difluorophenyl)-1-oxo-2-azaspiro[4.5]dec-8-yl]-N⁴-cyclopropyl-1H-imidazole-4,5-dicarboxamide
N-[(trans)-2-(2-chloro-4,5-difluorophenyl)-1-oxo-2-azaspiro[4.5]dec-8-yl]-4-[(3-fluoroazetidin-1-yl)carbonyl]-1H-imidazole-5-carboxamide
N⁵-[(trans)-2-(2-chloro-4,5-difluorophenyl)-1-oxo-2-azaspiro[4.5]dec-8-yl]-N⁴-(2,2,2-trifluoroethyl)-1H-imidazole-4,5-dicarboxamide
N⁵-[(trans)-2-(2-chloro-4,5-difluorophenyl)-1-oxo-2-azaspiro[4.5]dec-8-yl]-N⁴-(2-methoxyethyl)-1H-imidazole-4,5-dicarboxamide
N⁴-methyl-N⁵-{trans-4-[methyl(2-methylphenyl)carbamoyl]cyclohexyl}-1H-imidazole-4,5-dicarboxamide
N-{trans-4-[(2-chloro-4-fluorophenyl)carbamoyl]cyclohexyl}-7-cyclopropyl-5-methylpyrazolo[1,5-a]pyrimidine-3-carboxamide
N-{trans-4-[(2-chloro-4-fluorophenyl)carbamoyl]cyclohexyl}pyrazolo[1,5-a]pyridine-3-carboxamide
N-{trans-4-[(2-chloro-4-fluorophenyl)carbamoyl]cyclohexyl}imidazo[1,2-a]pyridine-3-carboxamide
N-[(cis)-2-(2-chloro-4-fluorophenyl)-1-oxo-2-azaspiro[4.5]dec-8-yl]-5,7-dimethylpyrazolo[1,5-a]pyrimidine-3-carboxamide
N-[(cis)-2-(4-fluorophenyl)-1-oxo-2-azaspiro[4.5]dec-8-yl]-5,7-dimethylpyrazolo[1,5-a]pyrimidine-3-carboxamide
N-[(trans)-2-(4-fluorophenyl)-1-oxo-2-azaspiro[4.5]dec-8-yl]pyrazolo[1,5-a]pyrimidine-3-carboxamide
N-[(trans)-2-(4-fluorophenyl)-1-oxo-2-azaspiro[4.5]dec-8-yl]-6-methylpyrazolo[1,5-a]pyrimidine-3-carboxamide
N-[(trans)-2-(4-fluorophenyl)-1-oxo-2-azaspiro[4.5]dec-8-yl]-5,7-dimethylpyrazolo[1,5-a]pyrimidine-3-carboxamide
N⁴-[(trans)-2-(4-fluorophenyl)-1-oxo-2-azaspiro[4.5]dec-8-yl]-N⁵-methyl-1H-imidazole-4,5-dicarboxamide
N⁵-ethyl-N⁴-[(trans)-2-(4-fluorophenyl)-1-oxo-2-azaspiro[4.5]dec-8-yl]-1H-imidazole-4,5-dicarboxamide
N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide
N-[(trans)-2-(2-chloro-4-fluorophenyl)-1-oxo-2-azaspiro[4.5]dec-8-yl]-5-(methylamino)pyrazolo[1,5-a]pyrimidine-3-carboxamide
N-[(trans)-2-(2-chloro-4-fluorophenyl)-1-oxo-2-azaspiro[4.5]dec-8-yl]-2-methylpyrazolo[1,5-a]pyrimidine-3-carboxamide
N-[(trans)-2-(2-chloro-4-fluorophenyl)-1-oxo-2-azaspiro[4.5]dec-8-yl]-5-(dimethylamino)pyrazolo[1,5-a]pyrimidine-3-carboxamide
N-[(trans)-2-(2-chloro-4-fluorophenyl)-1-oxo-2-azaspiro[4.5]dec-8-yl]-5-methoxypyrazolo[1,5-a]pyrimidine-3-carboxamide
N-[(trans)-2-(2-chloro-4-fluorophenyl)-1-oxo-2-azaspiro[4.5]dec-8-yl]-7-(methoxymethyl)-5-methylpyrazolo[1,5-a]pyrimidine-3-carboxamide
N-[(trans)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide
N-[(trans)-2-(2-chloro-4-fluorophenyl)-1-oxo-2-azaspiro[4.5]dec-8-yl]-5-methylpyrazolo[1,5-a]pyrimidine-3-carboxamide
N-[(trans)-2-(2-chloro-4-fluorophenyl)-1-oxo-2-azaspiro[4.5]dec-8-yl]-7-methylpyrazolo[1,5-a]pyrimidine-3-carboxamide
N-[(trans)-2-(2-chloro-4-fluorophenyl)-1-oxo-2-azaspiro[4.5]dec-8-yl]imidazo[1,2-a]pyridine-3-carboxamide
N-[(trans)-2-(2-chloro-4-fluorophenyl)-1-oxo-2-azaspiro[4.5]dec-8-yl]-7-(difluoromethyl)-5-methylpyrazolo[1,5-a]pyrimidine-3-carboxamide tert-butyl 4-(3-{[(trans)-2-(2-chloro-4-fluorophenyl)-1-oxo-2-azaspiro[4.5]dec-8-yl]carbamoyl}pyrazolo[1,5-a]pyrimidin-5-yl)piperazine-1-carboxylate
N-[(trans)-2-(2-chloro-4-fluorophenyl)-1-oxo-2-azaspiro[4.5]dec-8-yl]-5-(piperazin-1-yl)pyrazolo[1,5-a]pyrimidine-3-carboxamide hydrochloric acid salt
N-[(trans)-2-(2-chloro-4-fluorophenyl)-1-oxo-2-azaspiro[4.5]dec-8-yl]-2,5,7-trimethylpyrazolo[1,5-a]pyrimidine-3-carboxamide
N-[(trans)-2-(2-chloro-4-fluorophenyl)-1-oxo-2-azaspiro[4.5]dec-8-yl]-1H-pyrazolo[4,3-b]pyridine-3-carboxamide
N-[(trans)-2-(2-chloro-4-fluorophenyl)-1-oxo-2-azaspiro[4.5]dec-8-yl]-5-(4-methylpiperazin-1-yl)pyrazolo[1,5-a]pyrimidine-3-carboxamide
N-[(trans)-2-(2-chloro-4-fluorophenyl)-1-oxo-2-azaspiro[4.5]dec-8-yl]pyrazolo[1,5-a]pyridine-3-carboxamide
N-[(trans)-2-(2-chloro-4-fluorophenyl)-1-oxo-2-azaspiro[4.5]dec-8-yl]-5-(morpholin-4-yl)pyrazolo[1,5-a]pyrimidine-3-carboxamide
N-[(trans)-2-(2-chloro-4-fluorophenyl)-1-oxo-2-azaspiro[4.5]dec-8-yl]-5-(pyrrolidin-1-yl)pyrazolo[1,5-a]pyrimidine-3-carboxamide
N-[(trans)-2-(2-chloro-4-fluorophenyl)-1-oxo-2-azaspiro[4.5]dec-8-yl]-7-(trifluoromethyl)pyrazolo[1,5-a]pyrimidine-3-carboxamide
N-[(trans)-2-(2-chloro-4-fluorophenyl)-1-oxo-2-azaspiro[4.5]dec-8-yl]-5-hydroxypyrazolo[1,5-a]pyrimidine-3-carboxamide
N-[(trans)-2-(2-chloro-4-fluorophenyl)-1-oxo-2-azaspiro[4.5]dec-8-yl]-7-hydroxypyrazolo[1,5-a]pyrimidine-3-carboxamide
N-[(trans)-2-(2-chloro-4-fluorophenyl)-1-oxo-2-azaspiro[4.5]dec-8-yl]-1H-pyrrolo[3,2-b]pyridine-3-carboxamide
N-[(trans)-2-(2-chloro-4-fluorophenyl)-1-oxo-2-azaspiro[4.5]dec-8-yl]-6-(2-methoxyethyl)pyrazolo[1,5-a]pyrimidine-3-carboxamide
N-[(trans)-1-oxo-2-phenyl-2-azaspiro[4.5]dec-8-yl]pyrazolo[1,5-a]pyrimidine-3-carboxamide
5,7-dimethyl-N-[(trans)-1-oxo-2-phenyl-2-azaspiro[4.5]dec-8-yl]pyrazolo[1,5-a]pyrimidine-3-carboxamide
N-[(trans)-1-oxo-2-phenyl-2-azaspiro[4.5]dec-8-yl]imidazo[1,2-b]pyridazine-3-carboxamide
N-[(trans)-2-(2-chloro-4,6-difluorophenyl)-1-oxo-2-azaspiro[4.5]dec-8-yl]-7-(methoxymethyl)-5-methylpyrazolo[1,5-a]pyrimidine-3-carboxamide
N-[(trans)-2-(2-chloro-4,6-difluorophenyl)-1-oxo-2-azaspiro[4.5]dec-8-yl]-5-(methoxymethyl)-7-methylpyrazolo[1,5-a]pyrimidine-3-carboxamide
N-[(trans)-2-(2-chloro-4,6-difluorophenyl)-1-oxo-2-azaspiro[4.5]dec-8-yl]imidazo[1,2-b]pyridazine-3-carboxamide
N-[(trans)-2-(2-chloro-4,6-difluorophenyl)-1-oxo-2-azaspiro[4.5]dec-8-yl]pyrazolo[1,5-a]pyrimidine-3-carboxamide
N-[(trans)-2-(3,5-difluorophenyl)-1-oxo-2-azaspiro[4.5]dec-8-yl]pyrazolo[1,5-a]pyrimidine-3-carboxamide
N-[(trans)-2-(4-chloropyridin-3-yl)-1-oxo-2-azaspiro[4.5]dec-8-yl]pyrazolo[1,5-a]pyrimidine-3-carboxamide
N-[(trans)-2-(2-chloro-4-fluorophenyl)-1-oxo-2-azaspiro[4.5]dec-8-yl]-1-methyl-1H-indazole-3-carboxamide
N-[(trans)-2-(2-chloro-4-fluorophenyl)-1-oxo-2-azaspiro[4.5]dec-8-yl]-4-iodo-1H-indazole-3-carboxamide
N-{trans-4-[(2-chloro-4-fluorophenyl)carbamoyl]cyclohexyl}-1-methyl-1H-indazole-3-carboxamide
N-{trans-4-[(2-chloro-4-fluorophenyl)carbamoyl]cyclohexyl}-2-methyl-2H-indazole-3-carboxamide
N-[trans-2-(2-chloro-4-fluorophenyl)-1-oxo-2-azaspiro[4.5]dec-8-yl]-1H-indazole-3-carboxamide
N-{trans-4-[(2-chloro-4-fluorophenyl)carbamoyl]cyclohexyl}-1H-indole-3-carboxamide
N-{trans-4-[(2-chloro-4-fluorophenyl)carbamoyl]cyclohexyl}-1H-indazole-3-carboxamide
N-{trans-4-[(2-chloro-4-fluorophenyl)carbamoyl]cyclohexyl}-5-fluoro-1H-indazole-3-carboxamide
2-chloro-N-{trans-4-[(2-chloro-4-fluorophenyl)carbamoyl]cyclohexyl}imidazo[1,2-a]pyridine-3-carboxamide
N-[(trans)-2-(2-chlorophenyl)-1-oxo-2-azaspiro[4.5]dec-8-yl]pyrazolo[1,5-a]pyrimidine-3-carboxamide
N-[(trans)-2-(2-chlorophenyl)-1-oxo-2-azaspiro[4.5]dec-8-yl]-5,7-dimethylpyrazolo[1,5-a]pyrimidine-3-carboxamide
N⁵-[(trans)-2-(2-chlorophenyl)-1-oxo-2-azaspiro[4.5]dec-8-yl]-N⁴-ethyl-1H-imidazole-4,5-dicarboxamide
N⁵-[(trans)-2-(2-chlorophenyl)-1-oxo-2-azaspiro[4.5]dec-8-yl]-N⁴-(2-methoxyethyl)-1H-imidazole-4,5-dicarboxamide
N⁵-[(trans)-2-(2-chlorophenyl)-1-oxo-2-azaspiro[4.5]dec-8-yl]-N⁴-[2-(4-methylpiperidin-1-yl)ethyl]-1H-imidazole-4,5-dicarboxamide
N⁵-[(trans)-2-(2-chlorophenyl)-1-oxo-2-azaspiro[4.5]dec-8-yl]-1H-imidazole-4,5-dicarboxamide
N-[(trans)-2-(2-chlorophenyl)-1-oxo-2-azaspiro[4.5]dec-8-yl]-4-[(3-fluoroazetidin-1-yl)carbonyl]-1H-imidazole-5-carboxamide
N-[(trans)-2-(2-chlorophenyl)-1-oxo-2-azaspiro[4.5]dec-8-yl]-4-(2-oxa-6-azaspiro[3.3]hept-6-ylcarbonyl)-1H-imidazole-5-carboxamide
N⁵-[(trans)-2-(2-chlorophenyl)-1-oxo-2-azaspiro[4.5]dec-8-yl]-N⁴-methyl-1H-imidazole-4,5-dicarboxamide
N-[(trans)-2-(2-chloro-4-fluoro-5-methylphenyl)-1-oxo-2-azaspiro[4.5]dec-8-yl]imidazo[1,2-b]pyridazine-3-carboxamide
N-[(trans)-2-(2-chloro-4-fluoro-5-methylphenyl)-1-oxo-2-azaspiro[4.5]dec-8-yl]-5,7-dimethylpyrazolo[1,5-a]pyrimidine-3-carboxamide
N-[(trans)-2-(2-chloro-4-fluoro-5-methylphenyl)-1-oxo-2-azaspiro[4.5]dec-8-yl]pyrazolo[1,5-a]pyrimidine-3-carboxamide
N-[(trans)-2-(3-chlorophenyl)-1-oxo-2-azaspiro[4.5]dec-8-yl]pyrazolo[1,5-a]pyrimidine-3-carboxamide
N-[(trans)-2-(3-chlorophenyl)-1-oxo-2-azaspiro[4.5]dec-8-yl]imidazo[1,2-b]pyridazine-3-carboxamide
N-[(trans)-2-(3-chlorophenyl)-1-oxo-2-azaspiro[4.5]dec-8-yl]-5,7-dimethylpyrazolo[1,5-a]pyrimidine-3-carboxamide
N-[(trans)-2-(2-chlorophenyl)-1-oxo-2-azaspiro[4.5]dec-8-yl]imidazo[1,2-b]pyridazine-3-carboxamide
N-[(trans)-2-(2-chlorophenyl)-1-oxo-2-azaspiro[4.5]dec-8-yl]-1H-pyrazolo[4,3-b]pyridine-3-carboxamide
N-[(trans)-2-(2-chlorophenyl)-1-oxo-2-azaspiro[4.5]dec-8-yl]-7-methylpyrazolo[1,5-a]pyrimidine-3-carboxamide
N-[(trans)-2-(2-chlorophenyl)-1-oxo-2-azaspiro[4.5]dec-8-yl]-1H-pyrrolo[3,2-b]pyridine-3-carboxamide
N-[(trans)-2-(3-chlorophenyl)-1-oxo-2-azaspiro[4.5]dec-8-yl]-1H-pyrrolo[3,2-b]pyridine-3-carboxamide
N⁵-[(trans)-2-(3-chlorophenyl)-1-oxo-2-azaspiro[4.5]dec-8-yl]-N⁴-methyl-1H-imidazole-4,5-dicarboxamide
N⁵-[(trans)-2-(3-chlorophenyl)-1-oxo-2-azaspiro[4.5]dec-8-yl]-N⁴-ethyl-1H-imidazole-4,5-dicarboxamide
N⁵-[(trans)-2-(3-chlorophenyl)-1-oxo-2-azaspiro[4.5]dec-8-yl]-N⁴-(propan-2-yl)-1H-imidazole-4,5-dicarboxamide
N⁵-[(trans)-2-(3-chlorophenyl)-1-oxo-2-azaspiro[4.5]dec-8-yl]-N⁴-cyclopropyl-1H-imidazole-4,5-dicarboxamide
N-[(trans)-2-(3-chlorophenyl)-1-oxo-2-azaspiro[4.5]dec-8-yl]-4-[(3-fluoroazetidin-1-yl)carbonyl]-1H-imidazole-5-carboxamide
N⁵-[(trans)-2-(3-chlorophenyl)-1-oxo-2-azaspiro[4.5]dec-8-yl]-N⁴-(2,2,2-trifluoroethyl)-1H-imidazole-4,5-dicarboxamide
N⁵-[(trans)-2-(3-chlorophenyl)-1-oxo-2-azaspiro[4.5]dec-8-yl]-N⁴-(2-methoxyethyl)-1H-imidazole-4,5-dicarboxamide
N-[(trans)-2-(3-chlorophenyl)-1-oxo-2-azaspiro[4.5]dec-8-yl]-4-[(4-methylpiperazin-1-yl)carbonyl]-1H-imidazole-5-carboxamide
N⁵-[(trans)-2-(3-chlorophenyl)-1-oxo-2-azaspiro[4.5]dec-8-yl]-N⁴-(2-hydroxy-2-methylpropyl)-1H-imidazole-4,5-dicarboxamide
N⁵-[(trans)-2-(3-chlorophenyl)-1-oxo-2-azaspiro[4.5]dec-8-yl]-N⁴-(1-methoxypropan-2-yl)-1H-imidazole-4,5-dicarboxamide
N5-[(trans)-2-(3-chlorophenyl)-1-oxo-2-azaspiro[4.5]dec-8-yl]-N4-[(2S)-1-methoxypropan-2-yl]-1H-imidazole-4,5-dicarboxamide
N5-[(trans)-2-(3-chlorophenyl)-1-oxo-2-azaspiro[4.5]dec-8-yl]-N4-[(2R)-1-methoxypropan-2-yl]-1H-imidazole-4,5-dicarboxamide
N-[(trans)-2-(3-chlorophenyl)-1-oxo-2-azaspiro[4.5]dec-8-yl]-4-(morpholin-4-ylcarbonyl)-1H-imidazole-5-carboxamide
N⁵-[(trans)-2-(3-chlorophenyl)-1-oxo-2-azaspiro[4.5]dec-8-yl]-N⁴-[2-(morpholin-4-y)ethyl]-1H-imidazole-4,5-dicarboxamide
N⁵-[(trans)-2-(2-chloro-4-fluoro-5-methylphenyl)-1-oxo-2-azaspiro[4.5]dec-8-yl]-N⁴-methyl-1H-imidazole-4,5-dicarboxamide
N⁵-[(trans)-2-(2-chloro-4-fluoro-5-methylphenyl)-1-oxo-2-azaspiro[4.5]dec-8-yl]-N⁴-ethyl-1H-imidazole-4,5-dicarboxamide
N⁵-[(trans)-2-(2-chloro-4-fluoro-5-methylphenyl)-1-oxo-2-azaspiro[4.5]dec-8-yl]-N⁴-[2-(4-methylpiperidin-1-yl)ethyl]-1H-imidazole-4,5-dicarboxamide
N⁵-[(trans)-2-(2-chloro-4-fluoro-5-methylphenyl)-1-oxo-2-azaspiro[4.5]dec-8-yl]-1H-imidazole-4,5-dicarboxamide
N-[(trans)-2-(2-chloro-4-fluoro-5-methylphenyl)-1-oxo-2-azaspiro[4.5]dec-8-yl]-4-[(3-fluoroazetidin-1-yl)carbonyl]-1H-imidazole-5-carboxamide
N-[(trans)-2-(2-chloro-4-fluoro-5-methylphenyl)-1-oxo-2-azaspiro[4.5]dec-8-yl]-4-(2-oxa-6-azaspiro[3.3]hept-6-ylcarbonyl)-1H-imidazole-5-carboxamide
N-[(trans)-2-(2-chloro-4-fluoro-5-methylphenyl)-1-oxo-2-azaspiro[4.5]dec-8-yl]-4-[(4-methylpiperazin-1-yl)carbonyl]-1H-imidazole-5-carboxamide
N⁵-[(trans)-2-(2-chloro-4-fluoro-5-methylphenyl)-1-oxo-2-azaspiro[4.5]dec-8-yl]-N⁴-cyclopropyl-1H-imidazole-4,5-dicarboxamide
N-[(trans)-2-(2-chloro-4-fluoro-5-methylphenyl)-1-oxo-2-azaspiro[4.5]dec-8-yl]-4-{[3-(difluoromethoxy)azetidin-1-yl]carbonyl}-1H-imidazole-5-carboxamide
N⁵-[(trans)-2-(2-chloro-4-fluoro-5-methylphenyl)-1-oxo-2-azaspiro[4.5]dec-8-yl]-N⁴-(2,2,2-trifluoroethyl)-1H-imidazole-4,5-dicarboxamide
N-[(trans)-2-(2-chloro-4,5-difluorophenyl)-1-oxo-2-azaspiro[4.5]dec-8-yl]imidazo[1,2-b]pyridazine-3-carboxamide
N-[(trans)-2-(2-chloro-4,5-difluorophenyl)-1-oxo-2-azaspiro[4.5]dec-8-yl]-5,7-dimethylpyrazolo[1,5-a]pyrimidine-3-carboxamide
N-[(trans)-2-(2-chloro-4-fluoro-5-methylphenyl)-1-oxo-2-azaspiro[5.5]undec-9-yl]pyrazolo[1,5-a]pyrimidine-3-carboxamide
N-[(trans)-2-(2-chloro-4-fluoro-5-methylphenyl)-1-oxo-2-azaspiro[5.5]undec-9-yl]imidazo[1,2-b]pyridazine-3-carboxamide
N-[(trans)-2-(2-chloro-4-fluoro-5-methylphenyl)-1-oxo-2-azaspiro[5.5]undec-9-yl]-5,7-dimethylpyrazolo[1,5-a]pyrimidine-3-carboxamide
N-[(trans)-2-(2-chloro-4-fluoro-5-methylphenyl)-1-oxo-2-azaspiro[5.5]undec-9-yl]-4-[(3-fluoroazetidin-1-yl)carbonyl]-1H-imidazole-5-carboxamide
N⁵-[(trans)-2-(2-chloro-4-fluoro-5-methylphenyl)-1-oxo-2-azaspiro[5.5]undec-9-yl]-N⁴-methyl-1H-imidazole-4,5-dicarboxamide
N⁵-[(trans)-2-(2-chloro-4-fluoro-5-methylphenyl)-1-oxo-2-azaspiro[5.5]undec-9-yl]-N⁴-ethyl-1H-imidazole-4,5-dicarboxamide
N⁵-[(trans)-2-(2-chloro-4-fluoro-5-methylphenyl)-1-oxo-2-azaspiro[5.5]undec-9-yl]-N⁴-(2-methoxyethyl)-1H-imidazole-4,5-dicarboxamide
N⁴-[(trans)-2-(2-chloro-4-fluorophenyl)-3-hydroxy-1-oxo-2-azaspiro[4.5]dec-8-yl]-N⁵-methyl-1H-imidazole-4,5-dicarboxamide
N⁴-[(3R,trans)-2-(2-chloro-4-fluorophenyl)-3-hydroxy-1-oxo-2-azaspiro[4.5]dec-8-yl]-N⁵-methyl-1H-imidazole-4,5-dicarboxamide
N⁴-[(3S,trans)-2-(2-chloro-4-fluorophenyl)-3-hydroxy-1-oxo-2-azaspiro[4.5]dec-8-yl]-N⁵-methyl-1H-imidazole-4,5-dicarboxamide
N⁵-[(trans)-2-(2-chloro-4-fluoro-5-methylphenyl)-1-oxo-2-azaspiro[5.5]undec-9-yl]-N⁴-cyclopropyl-1H-imidazole-4,5-dicarboxamide
N⁵-[(trans)-2-(2-chloro-4-fluoro-5-methylphenyl)-1-oxo-2-azaspiro[5.5]undec-9-yl]-1H-imidazole-4,5-dicarboxamide
N⁵-[(trans)-2-(2-chloro-4-fluoro-5-methylphenyl)-1-oxo-2-azaspiro[5.5]undec-9-yl]-N⁴-(propan-2-yl)-1H-imidazole-4,5-dicarboxamide
N-[(trans)-2-(2-chloro-4-fluoro-5-methylphenyl)-1-oxo-2-azaspiro[5.5]undec-9-yl]-4-(2-oxa-6-azaspiro[3.3]hept-6-ylcarbonyl)-1H-imidazole-5-carboxamide
N⁵-[(trans)-2-(2-chloro-4-fluoro-5-methylphenyl)-1-oxo-2-azaspiro[5.5]undec-9-yl]-N⁴-[2-(4-methylpiperidin-1-yl)ethyl]-1H-imidazole-4,5-dicarboxamide
N-[(trans)-2-(2-chloro-4,5-difluorophenyl)-1-oxo-2-azaspiro[5.5]undec-9-yl]pyrazolo[1,5-a]pyrimidine-3-carboxamide
N-[(trans)-2-(2-chloro-4,5-difluorophenyl)-1-oxo-2-azaspiro[5.5]undec-9-yl]imidazo[1,2-b]pyridazine-3-carboxamide
N-[(trans)-2-(2-chloro-4,5-difluorophenyl)-1-oxo-2-azaspiro[5.5]undec-9-yl]-5,7-dimethylpyrazolo[1,5-a]pyrimidine-3-carboxamide
N-[(trans)-2-(2-chloro-4,5-difluorophenyl)-1-oxo-2-azaspiro[5.5]undec-9-yl]-4-[(3-fluoroazetidin-1-yl)carbonyl]-1H-imidazole-5-carboxamide
N⁵-[(trans)-2-(2-chloro-4,5-difluorophenyl)-1-oxo-2-azaspiro[5.5]undec-9-yl]-N⁴-methyl-1H-imidazole-4,5-dicarboxamide
N⁵-[(trans)-2-(2-chloro-4,5-difluorophenyl)-1-oxo-2-azaspiro[5.5]undec-9-yl]-N⁴-ethyl-1H-imidazole-4,5-dicarboxamide
6-bromo-N-[(trans)-2-(2-chloro-4-fluorophenyl)-1-oxo-2-azaspiro[4.5]dec-8-yl]-1-isopropyl-1H-indazole-3-carboxamide
N⁵-[(trans)-2-(2-chloro-4,5-difluorophenyl)-1-oxo-2-azaspiro[5.5]undec-9-yl]-1H-imidazole-4,5-dicarboxamide
N⁵-[(trans)-2-(2-chloro-4,5-difluorophenyl)-1-oxo-2-azaspiro[5.5]undec-9-yl]-N⁴-(propan-2-yl)-1H-imidazole-4,5-dicarboxamide
N-[(trans)-2-(2-chloro-4,5-difluorophenyl)-1-oxo-2-azaspiro[5.5]undec-9-yl]-4-{[3-(difluoromethoxy)azetidin-1-yl]carbonyl}-1H-imidazole-5-carboxamide
N-[(trans)-2-(2-chloro-4,5-difluorophenyl)-1-oxo-2-azaspiro[5.5]undec-9-yl]-4-(morpholin-4-ylcarbonyl)-1H-imidazole-5-carboxamide
N⁵-[(trans)-2-(2-chloro-4,5-difluorophenyl)-1-oxo-2-azaspiro[5.5]undec-9-yl]-N⁴-cyclopropyl-1H-imidazole-4,5-dicarboxamide
N-[(trans)-2-(2-chloro-4-fluorophenyl)-1-oxo-2-azaspiro[5.5]undec-9-yl]pyrazolo[1,5-a]pyrimidine-3-carboxamide
N-[(trans)-2-(2-chloro-4-fluorophenyl)-1-oxo-2-azaspiro[5.5]undec-9-yl]imidazo[1,2-b]pyridazine-3-carboxamide
N-[(trans)-2-(2-chloro-4-fluorophenyl)-1-oxo-2-azaspiro[5.5]undec-9-yl]-4-[(3-fluoroazetidin-1-yl)carbonyl]-1H-imidazole-5-carboxamide
N⁵-[(trans)-2-(2-chloro-4-fluorophenyl)-1-oxo-2-azaspiro[5.5]undec-9-yl]-N⁴-cyclopropyl-1H-imidazole-4,5-dicarboxamide
N⁵-[(trans)-2-(2-chloro-4-fluorophenyl)-1-oxo-2-azaspiro[5.5]undec-9-yl]-N⁴-(propan-2-yl)-1H-imidazole-4,5-dicarboxamide
N-[(trans)-2-(2-chloro-4-fluorophenyl)-1-oxo-2-azaspiro[4.5]dec-8-yl]-5-(difluoromethyl)pyrazolo[1,5-a]pyrimidine-3-carboxamide
N-[(trans)-2-(2-chloro-4-fluorophenyl)-1-oxo-2-azaspiro[4.5]dec-8-yl]-5,7-bis(difluoromethyl)pyrazolo[1,5-a]pyrimidine-3-carboxamide
N-[(cis)-2-(2-chloro-4-fluorophenyl)-1-oxo-2-azaspiro[4.5]dec-8-yl]-1H-pyrrolo[3,2-b]pyridine-3-carboxamide
N-[(cis)-2-(2-chloro-4-fluorophenyl)-1-oxo-2-azaspiro[4.5]dec-8-yl]-7-(difluoromethyl)-5-methylpyrazolo[1,5-a]pyrimidine-3-carboxamide
N⁵-[(trans)-2-(2-chloro-4-fluoro-5-methylphenyl)-1-oxo-2-azaspiro[4.5]dec-8-yl]-N⁴-(2-methoxyethyl)-1H-imidazole-4,5-dicarboxamide
N-[(cis)-2-(2-chloro-4-fluorophenyl)-1-oxo-2-azaspiro[4.5]dec-8-yl]-5-methylpyrazolo[1,5-a]pyrimidine-3-carboxamide
N-[(cis)-2-(2-chloro-4-fluorophenyl)-1-oxo-2-azaspiro[4.5]dec-8-yl]-7-methylpyrazolo[1,5-a]pyrimidine-3-carboxamide
N-[(cis)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide
N⁵-[(cis)-2-(2-chloro-4-fluorophenyl)-1-oxo-2-azaspiro[4.5]dec-8-yl]-N⁴-cyclopropyl-1H-imidazole-4,5-dicarboxamide
N⁵-[(cis)-2-(2-chloro-4-fluorophenyl)-1-oxo-2-azaspiro[4.5]dec-8-yl]-N⁴-ethyl-1H-imidazole-4,5-dicarboxamide
N-[(cis)-2-(2-chloro-4-fluorophenyl)-1-oxo-2-azaspiro[4.5]dec-8-yl]-4-[(3-fluoroazetidin-1-yl)carbonyl]-1H-imidazole-5-carboxamide
N⁵-[(cis)-2-(2-chloro-4-fluorophenyl)-1-oxo-2-azaspiro[4.5]dec-8-yl]-N⁴-(2-methoxyethyl)-1H-imidazole-4,5-dicarboxamide
N⁵-[(cis)-2-(2-chloro-4-fluorophenyl)-1-oxo-2-azaspiro[4.5]dec-8-yl]-1H-imidazole-4,5-dicarboxamide
N-[(trans)-2-(2-chloro-4-fluorophenyl)-1-oxo-2-azaspiro[4.5]dec-8-yl]-1-methyl-1H-pyrrolo[3,2-b]pyridine-3-carboxamide
N-[(trans)-2-(2-chloro-4-fluorophenyl)-1-oxo-2-azaspiro[4.5]dec-8-yl]-1-(2-hydroxyethyl)-1H-pyrrolo[3,2-b]pyridine-3-carboxamide
N-[(trans)-2-(2-chloro-4-fluorophenyl)-1-oxo-2-azaspiro[4.5]dec-8-yl]-1-(2-methoxyethyl)-1H-pyrrolo[3,2-b]pyridine-3-carboxamide
N-[(trans)-1-oxo-2-(pyridin-2-yl)-2-azaspiro[4.5]dec-8-yl]pyrazolo[1,5-a]pyrimidine-3-carboxamide
N-[(trans)-2-(2-chloro-4-fluorophenyl)-1-oxo-2-azaspiro[4.5]dec-8-yl][1,2,4]triazolo[4,3-b]pyridazine-3-carboxamide
N-[(trans)-2-(2-chloro-4-fluorophenyl)-1-oxo-2-azaspiro[4.5]dec-8-yl]-5,7-diethylpyrazolo[1,5-a]pyrimidine-3-carboxamide
6-chloro-N-[(trans)-2-(2-chloro-4-fluorophenyl)-1-oxo-2-azaspiro[4.5]dec-8-yl]-8-[(3,3,3-trifluoropropyl)amino]imidazo[1,2-b]pyridazine-3-carboxamide
N-[(trans)-2-(2-chloro-4-fluorophenyl)-1-oxo-2-azaspiro[4.5]dec-8-yl]-5H-pyrrolo[2,3-b]pyrazine-7-carboxamide
methyl 3-({trans-4-[(2-chloro-4,6-difluorophenyl)carbamoyl]cyclohexyl}carbamoyl)-5-methylpyrazolo[1,5-a]pyrimidine-7-carboxylate
methyl 3-({trans-4-[(2-chloro-4-fluorophenyl)carbamoyl]cyclohexyl}carbamoyl)-5-methylpyrazolo[1,5-a]pyrimidine-7-carboxylate
N-[(trans)-2-(2-chloro-5-methoxyphenyl)-1-oxo-2-azaspiro[4.5]dec-8-yl]pyrazolo[1,5-a]pyrimidine-3-carboxamide
N-{(trans)-2-[2-chloro-4-(trifluoromethoxy)phenyl]-1-oxo-2-azaspiro[4.5]dec-8-yl}pyrazolo[1,5-a]pyrimidine-3-carboxamide
N-[(trans)-2-(2-chloro-4-fluorophenyl)-3-methyl-1-oxo-2-azaspiro[4.5]dec-8-yl]pyrazolo[1,5-a]pyrimidine-3-carboxamide (mixture of isomers at C3)
N-[(3S,trans)-2-(2-chloro-4-fluorophenyl)-3-methyl-1-oxo-2-azaspiro[4.5]dec-8-yl]pyrazolo[1,5-a]pyrimidine-3-carboxamide
N-[(3R,trans)-2-(2-chloro-4-fluorophenyl)-3-methyl-1-oxo-2-azaspiro[4.5]dec-8-yl]pyrazolo[1,5-a]pyrimidine-3-carboxamide
N-[(trans)-2-(2-chloro-4-fluorophenyl)-1-oxo-2-azaspiro[4.5]dec-8-yl]-6-[1-(dimethylamino)ethyl]-7-methylpyrazolo[1,5-a]pyrimidine-3-carboxamide
N-[(trans)-2-(2-chloro-4-fluorophenyl)-1-oxo-2-azaspiro[4.5]dec-8-yl]-6-[(1R)-1-(dimethylamino)ethyl]-7-methylpyrazolo[1,5-a]pyrimidine-3-carboxamide
N-[(trans)-2-(2-chloro-4-fluorophenyl)-1-oxo-2-azaspiro[4.5]dec-8-yl]-6-[(1 S)-1-(dimethylamino)ethyl]-7-methylpyrazolo[1,5-a]pyrimidine-3-carboxamide
N-{trans-4-[(2-chloro-4-fluorophenyl)carbamoyl]cyclohexyl}-6-(2-hydroxypropan-2-yl)-7-methylpyrazolo[1,5-a]pyrimidine-3-carboxamide
N-{trans-4-[(2-chloro-4-fluorophenyl)carbamoyl]cyclohexyl}-6-hydroxyimidazo[1,2-b]pyridazine-3-carboxamide
N-[(trans)-1-oxo-2-(thiophen-2-yl)-2-azaspiro[4.5]dec-8-yl]pyrazolo[1,5-a]pyrimidine-3-carboxamide
methyl 3-chloro-4-{(trans)-1-oxo-8-[(pyrazolo[1,5-a]pyrimidin-3-ylcarbonyl)amino]-2-azaspiro[4.5]dec-2-yl}benzoate
N-{trans-4-[(2-chloro-4,6-difluorophenyl)carbamoyl]cyclohexyl}-5-methyl-7,8-dihydro-6H-cyclopenta[e]pyrazolo[1,5-a]pyrimidine-3-carboxamide
N-[(trans)-2-(2-chloro-4-fluorophenyl)-1-oxo-2-azaspiro[4.5]dec-8-yl]-7-hydroxy-5-methylpyrazolo[1,5-a]pyrimidine-3-carboxamide
N-{4-[(2-chloro-4-fluorophenyl)carbamoyl]-4-fluorocyclohexyl}pyrazolo[1,5-a]pyrimidine-3-carboxamide (Single isomer)
N⁴-{4-[(2-chloro-4-fluorophenyl)carbamoyl]-4-fluorocyclohexyl}-N⁵-methyl-1H-imidazole-4,5-dicarboxamide (Single isomer)
   or a stereoisomer, a tautomer, an N-oxide, a hydrate, a solvate, or a salt thereof, or a mixture of same.

In a further aspect, the invention relates to compounds of formula (I) supra, wherein:

A represents a group selected from:

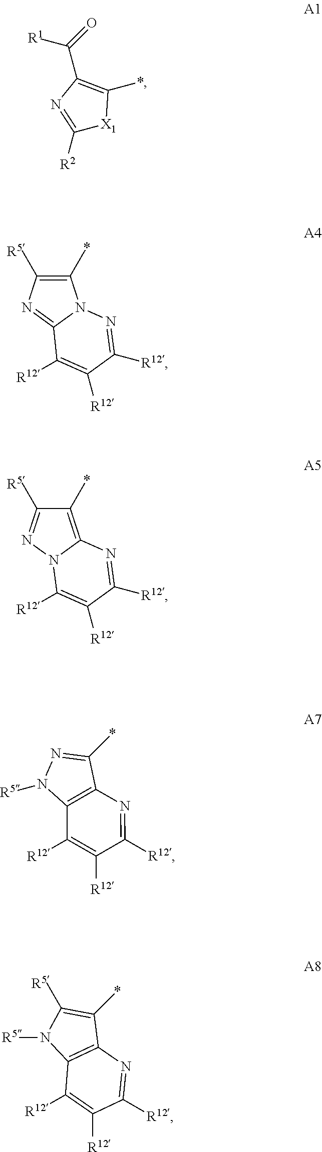

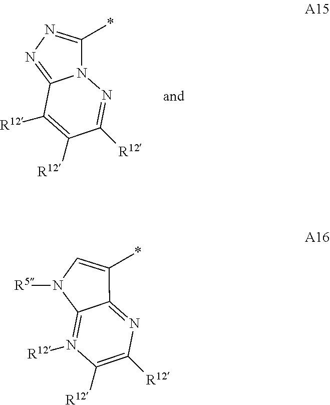

wherein * indicates the point of attachment of said groups with the rest of the molecule,
wherein said point of attachment is a carbon atom;
X¹ represents NR³ or 0,
R¹ represents a group selected from
—OR¹³, and —N(R¹⁴)R¹⁵,
   R² represents hydrogen,
   R³ represents a hydrogen atom,
   R⁴ represents a hydrogen atom,
   R^{5′} represents a group selected from:
   hydrogen, halogen, hydroxy, C₁-C₃-alkyl and —NH₂,
   R^{5′} represents, independently of each other, a group selected from:
   hydrogen, C₁-alkyl, C₂-hydroxyalkyl and (C₁-alkoxy)-(C₂-alkyl)-,
   R⁶ represents hydrogen, fluorine, or C₁-alkyl;
   R⁷ represents hydrogen;
   or
   R⁶, R⁷ represent fluorine;
   in embodiment a), R⁸, R⁹ and R¹⁰ represent:
   R⁸ represents hydrogen,
   R⁹ represents hydrogen, or C₁-alkyl; or
   R⁸ and R⁹ together represent a group:

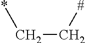

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, or C₂-C₃-hydroxyalkyl; or
in embodiment b), R⁸, R⁹ and R¹⁰ represent:
R⁸ represents hydrogen,
R⁹ and R¹⁰ together represent a group selected from:

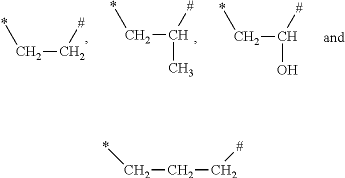

wherein * indicates the point of attachment of said group to the rest of the molecule at R⁹, and ^{#} indicates the point of attachment of said group to the rest of the molecule at R¹⁰;
R¹¹ represents a group selected from:
phenyl, pyridyl and pyrimidinyl,
   wherein phenyl, pyridyl and pyrimidinyl groups are optionally substituted with one, two or three groups, which are independently of each other selected from:
   C₁-C₄-alkyl, C₁-C₃-alkoxy, C₁-C₃-hydroxyalkyl, C₁-C₃-haloalkyl, halogen (preferably selected from chlorine and fluorine) and cyano,
      R^{12′} represents, independently of each other, hydrogen, chlorine, fluorine hydroxy, C₁-C₂-alkyl, C₃-C₄-cycloalkyl, C₁-alkoxy, —N(R¹⁸)R¹⁹, —C(O)R¹³, or —C(O)OR¹³,
      wherein C₁-C₂-alkyl is optionally substituted one, two or three times, independently of each other, with halogen or optionally substituted one time with a substituent selected from hydroxy, C₁-alkoxy, —NH₂, —NH(CH₃), —N(CH₃)₂,
      whereby two substituents R^{12′} when they are in adjacent positions of the ring to which they are attached, can be linked to one another in such a way that they jointly form propane-1,3-diyl;
      R¹³ represents C₁-alkyl,
      R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₃-hydroxyalkyl, (C₁-alkoxy)-(C₂-C₃-alkyl)-, C₁-C₃-haloalkyl, (C₁-alkyl)₂N(C₂-C₃-alkyl)-, 4- to 6-membered heterocycloalkyl (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
   C₁-alkyl, C₁-haloalkyl, C₁-alkoxy, C₁-haloalkoxy, —N(CH₃)₂, hydroxy and a halogen atom,
      whereby when two substituents are attached to the same ring carbon atom, together with the carbon atom to which they are attached, can be linked to one another in such a way that they jointly form a azetidine, thietane 1,1-dioxide, or oxetane;
      said azetidine being optionally substituted one time with C₁-alkyl or C₁-haloalkyl,
      R¹⁸ and R¹⁹ are, independently of each other, selected from:
   hydrogen, C₁-C₃-alkyl, C₂-C₃-hydroxyalkyl, and C₁-C₃-haloalkyl,
      or,
R¹⁸ and R¹⁹ together with the nitrogen atom to which they are attached form a 5- to 6-membered heterocycloalkyl group, in which one carbon atom is optionally replaced by a further heteroatom-containing group selected from NR²⁰, O,
   R²⁰ represents, independently of each other, a group selected from:
   hydrogen and C₁-alkyl,
      or a stereoisomer, a tautomer, an N-oxide, a hydrate, a solvate, or a salt thereof, or a mixture of same.

In a further embodiment of the above-mentioned aspects, the invention relates to compounds of formula (I), wherein:

A represents a group selected from:

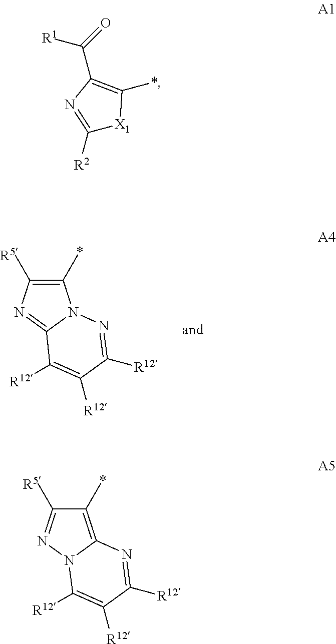

wherein * indicates the point of attachment of said groups with the rest of the molecule,
wherein said point of attachment is a carbon atom;
X¹ represents NR³ or O,
R¹ represents a group selected from
—OR¹³, and —N(R¹⁴)R¹⁵,
   R² represents hydrogen,
   R³ represents a hydrogen atom,
   R⁴ represents a hydrogen atom,
   R^{5′} represents a group selected from:
   hydrogen, halogen, hydroxy, C₁-C₃-alkyl and —NH₂,
   R⁶ represents hydrogen, fluorine, C₁-alkyl or C₁-alkoxy;
   R⁷ represents hydrogen; or
   R⁶, R⁷ represent fluorine;
   in embodiment a), R⁸, R⁹ and R¹⁰ represent:
   R⁸ represents hydrogen,
   R⁹ represents hydrogen, or C₁-alkyl; or
   R⁸ and R⁹ together represent a group:

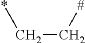

wherein * indicates the point of attachment of said group to the rest of the molecule at R⁸,
and ^{#} indicates the point of attachment of said group to the rest of the molecule at R⁹,
R¹⁰ represents hydrogen, C₁-C₃-alkyl, (C₃-C₄-cycloalkyl)-(C₁-C₃-alkyl)-, or C₂-C₃-hydroxyalkyl; or
in embodiment b), R⁸, R⁹ and R¹⁰ represent:
R⁸ represents hydrogen,
R⁹ and R¹⁰ together represent a group selected from:

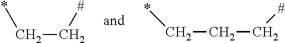

wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
   wherein aryl and heteroaryl groups are optionally substituted with one, two or three groups, which are independently of each other selected from:
   C₁-C₄-alkyl, C₁-C₃-alkoxy, C₁-C₃-hydroxyalkyl, C₁-C₃-haloalkyl, halogen and, cyano,
      R^{12′} represents, independently of each other, hydrogen, halogen, hydroxy, C₁-C₃-alkyl, C₃-C₄-cycloalkyl, C₁-alkoxy, —N(R¹⁸)R¹⁹, —C(O)R¹³, or —C(O)OR¹³,
      wherein C₁-C₃-alkyl is optionally substituted one, two or three times, independently of each other, with halogen and optionally substituted one time with a substituent selected from hydroxy, C₁-alkoxy, —NH₂, —NH(CH₃), —N(CH₃)₂,
      whereby two substituents R^{12′} when they are in adjacent positions of the ring to which they are attached, can be linked to one another in such a way that they jointly form propane-1,3-diyl;
      R¹³ represents C₁-alkyl,
      R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₃-hydroxyalkyl, (C₁-alkoxy)-(C₂-C₃-alkyl)-, C₁-C₃-haloalkyl, (C₁-alkyl)₂N(C₂-C₃-alkyl)-(4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
      C₁-alkoxy, —N(CH₃)₂, and a halogen atom,
      whereby when two substituents are attached to the same ring carbon atom, together with the carbon atom to which they are attached, can be linked to one another in such a way that they jointly form a azetidine, thietane 1,1-dioxide, or oxetane;
      said azetidine being optionally substituted one time with C₁-alkyl or C₁-haloalkyl,
      R¹⁸ and R¹⁹ are, independently of each other, selected from:
   hydrogen and C₁-alkyl,
      or,
R¹⁸ and R¹⁹ together with the nitrogen atom to which they are attached form a 5- to 6-membered heterocycloalkyl group, in which one carbon atom is optionally replaced by a further heteroatom-containing group selected from NR²⁰, 0,
   R²⁰ represents, independently of each other, a group selected from:
   hydrogen and C₁-alkyl,
      or a stereoisomer, a tautomer, an N-oxide, a hydrate, a solvate, or a salt thereof, or a mixture of same.

In a further embodiment of the above-mentioned aspects, the invention relates to compounds of formula (I), wherein the compound of formula (I) has the cis configuration:

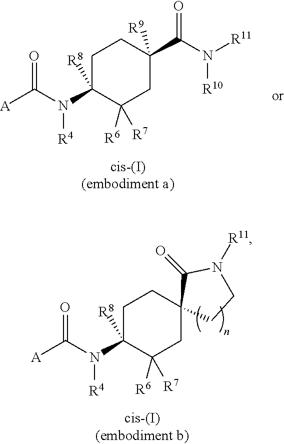

wherein n is 1 or 2.

In a further embodiment of the above-mentioned aspects, the invention relates to compounds of formula (I), wherein the compound of formula (I) has the cis configuration:

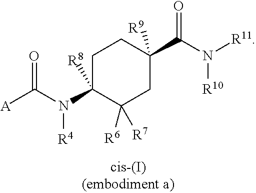

In a further embodiment of the above-mentioned aspects, the invention relates to compounds of formula (I), wherein the compound of formula (I) has the cis configuration:

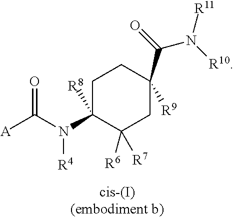

In a further embodiment of the above-mentioned aspects, the invention relates to compounds of formula (I), wherein the compound of formula (I) has the cis configuration:

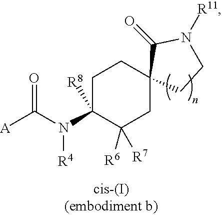

in which n is 1 or 2.

In a further embodiment of the above-mentioned aspects, the invention relates to compounds of formula (I), wherein the compound of formula (I) has the trans configuration:

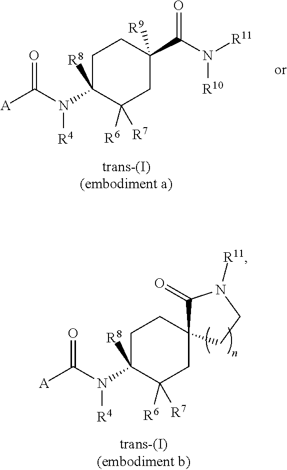

wherein n is 1 or 2.

In a further embodiment of the above-mentioned aspects, the invention relates to compounds of formula (I), wherein the compound of formula (I) has the trans configuration:

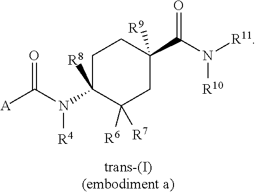

In a further embodiment of the above-mentioned aspects, the invention relates to compounds of formula (I), wherein the compound of formula (I) has the trans configuration:

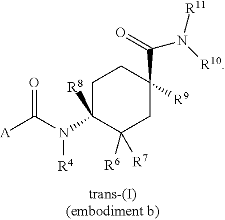

In a further embodiment of the above-mentioned aspects, the invention relates to compounds of formula (I), wherein the compound of formula (I) has the trans configuration:

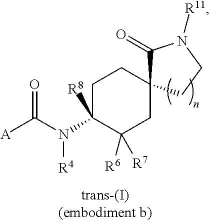

in which n is 1 or 2.

In a further embodiment of the above-mentioned aspects, the invention relates to compounds of formula (I), wherein:

A represents a group selected from:

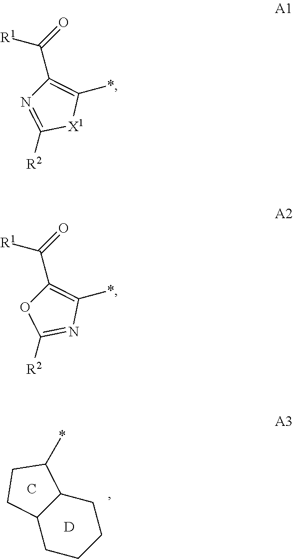

wherein * indicates the point of attachment of said groups with the rest of the molecule, wherein said point of attachment is a carbon atom;

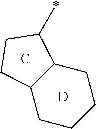

represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by one or two N atoms,
said ring C being optionally substituted with one or two R⁵ groups, and
ring D represents a phenyl group or a 6-membered heteroaryl group which contains one, two or three nitrogen heteroatoms, said ring D being optionally substituted with one, two or three R¹² groups,
in which R¹, R², X¹, R⁵ and R¹² are as defined herein.

In a further embodiment of the above-mentioned aspects, the invention relates to compounds of formula (I), wherein:

A represents a group A1:

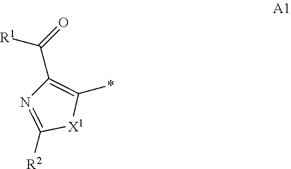

wherein * indicates the point of attachment of said group with the rest of the molecule,
in which R¹, R², and X¹ are as defined herein.

In a further embodiment of the above-mentioned aspects, the invention relates to compounds of formula (I), wherein:

A represents a group A1:

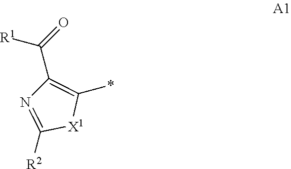

wherein * indicates the point of attachment of said group with the rest of the molecule,
in which X¹ represents NH and R¹ and R² are as defined herein.

In a further embodiment of the above-mentioned aspects, the invention relates to compounds of formula (I), wherein:

A represents a group A2:

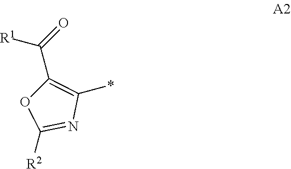

wherein * indicates the point of attachment of said group with the rest of the molecule,
in which R¹, and R² are as defined herein.

In a further embodiment of the above-mentioned aspects, the invention relates to compounds of formula (I), wherein:

A represents a group A3:

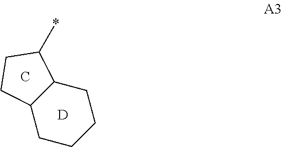

wherein * indicates the point of attachment of said groups with the rest of the molecule,
wherein said point of attachment is a carbon atom;

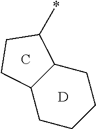

represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by one or two N atoms,
said ring C being optionally substituted with one or two R⁵ groups, and
ring D represents a phenyl group or a 6-membered heteroaryl group which contains one, two or three nitrogen heteroatoms, said ring D being optionally substituted with one, two or three R¹² groups,
in which R⁵ and R¹² are as defined herein.

In a further embodiment of the above-mentioned aspects, the invention relates to compounds of formula (I), wherein:

A represents a group A3:

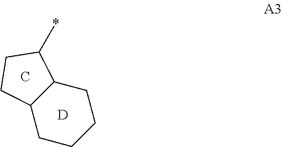

wherein * indicates the point of attachment of said groups with the rest of the molecule,
wherein said point of attachment is a carbon atom;

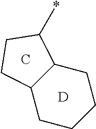

represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by one or two N atoms,
said ring C being optionally substituted with one or two R^{5′} groups, and
ring D represents a phenyl group or a 6-membered heteroaryl group which contains one, two or three nitrogen heteroatoms, said ring D being optionally substituted with one, two or three R^{12′} groups,
in which R^{5′} and R^{12′} are as defined herein, with the proviso that R^{5′} and R^{12′} are not hydrogen.

In a further embodiment of the above-mentioned aspects, the invention relates to compounds of formula (I), wherein:

A represents a group A6:

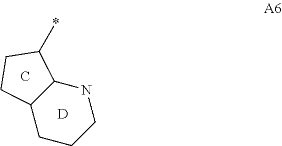

wherein * indicates the point of attachment of said groups with the rest of the molecule,
wherein said point of attachment is a carbon atom;

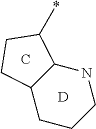

represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R⁵ groups, and
ring D represents a 6-membered heteroaryl group which contains one, two or three nitrogen heteroatoms, said ring being optionally substituted with one, two or three R¹² groups,
in which R⁵ and R¹² are as defined herein.

In a further embodiment of the above-mentioned aspects, the invention relates to compounds of formula (I), wherein:

A represents a group A6:

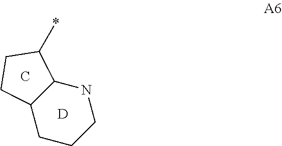

wherein * indicates the point of attachment of said groups with the rest of the molecule,
wherein said point of attachment is a carbon atom;

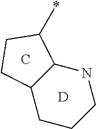

represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R^{5′} groups, and
ring D represents a 6-membered heteroaryl group which contains one, two or three nitrogen heteroatoms, said ring being optionally substituted with one, two or three R^{12′} groups,
in which R^{5′} and R^{12′} are as defined herein, with the proviso that R^{5′} and R^{12′} are not hydrogen.

In a further embodiment of the above-mentioned aspects, the invention relates to compounds of formula (I), wherein:

A represents a group selected from:

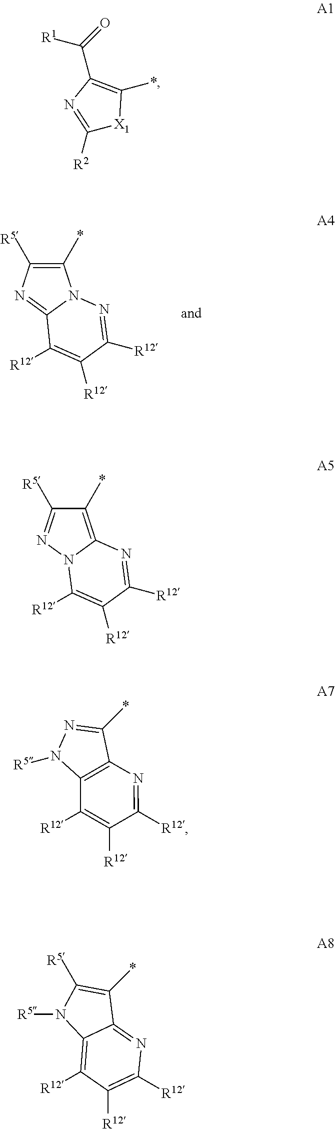

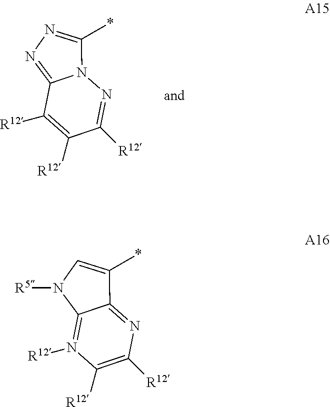

wherein * indicates the point of attachment of said groups with the rest of the molecule,
in which R^{5′}, and R⁵″, R^{12′} are as defined herein.

In a further embodiment of the above-mentioned aspects, the invention relates to compounds of formula (I), wherein:

A represents a group selected from:

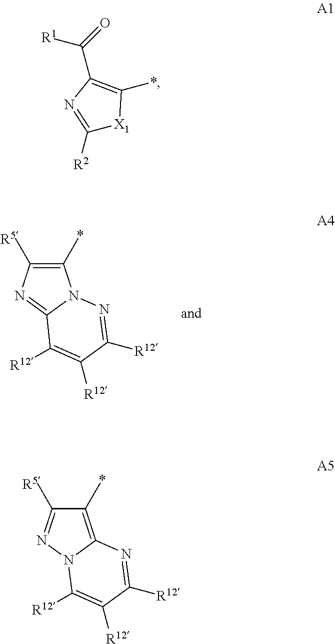

wherein * indicates the point of attachment of said groups with the rest of the molecule,
in which R^{5′} and R^{12′} are as defined herein.

In a further embodiment of the above-mentioned aspects, the invention relates to compounds of formula (I), wherein:

A represents a group selected from:

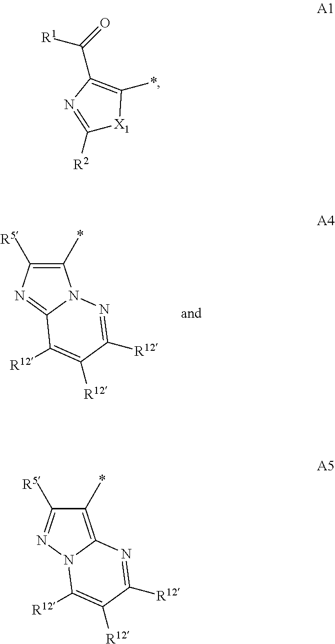

X¹ represents NH,
R^{5′} represents a group selected from:
hydrogen, halogen, hydroxy, C₁-C₃-alkyl and —NH₂,
R^{12′} represents, independently of each other, hydrogen, halogen, hydroxy, C₁-C₄-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
wherein C₁-C₄-alkyl is optionally substituted one, two or three times, independently of each other, with halogen and optionally substituted one time with a substituent selected from hydroxy, C-alkoxy, —NH₂, —NH(CH₃), —N(CH₃)₂,
whereby two substituents R^{12′} when they are in adjacent positions of the ring to which they are attached, can be linked to one another in such a way that they jointly form propane-1,3-diyl.

In a further embodiment of the above-mentioned aspects, the invention relates to compounds of formula (I), wherein:

A represents a group selected from:

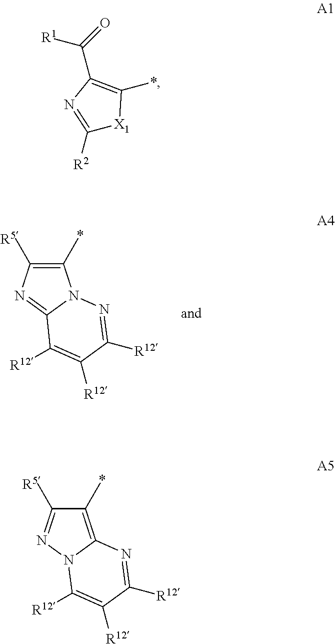

X¹ represents NH
R^{5′} represents a group selected from:
hydrogen, halogen, hydroxy, C₁-C₃-alkyl and —NH₂,
R^{12′} represents, independently of each other, hydrogen, halogen, hydroxy, C₁-C₄-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
wherein C₁-C₄-alkyl is optionally substituted one, two or three times, independently of each other, with halogen and optionally substituted one time with a substituent selected from hydroxy, C₁-alkoxy, —NH₂, —NH(CH₃), —N(CH₃)₂,
whereby two substituents R^{12′} when they are in adjacent positions of the ring to which they are attached, can be linked to one another in such a way that they jointly form propane-1,3-diyl.

In a further embodiment of the above-mentioned aspects, the invention relates to compounds of formula (I), wherein:

A represents a group selected from:

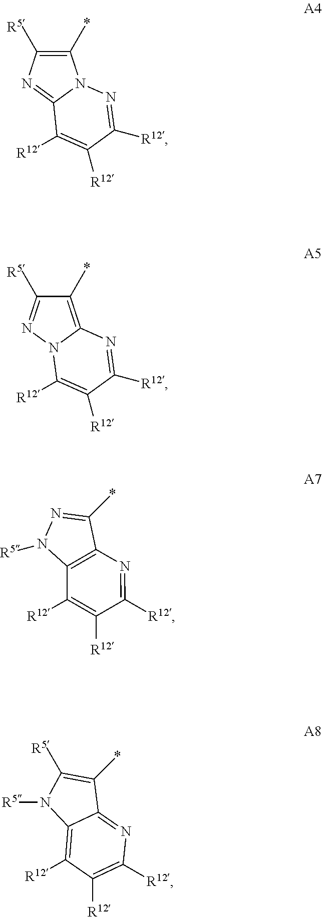

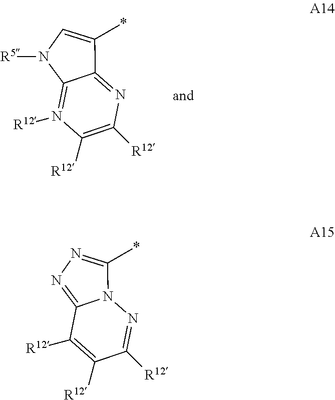

R^{5′} represents a group selected from:
hydrogen, halogen, hydroxy, C₁-C₃-alkyl and —NH₂,
R^{5″} represents, independently of each other, a group selected from:
hydrogen, C₁-C₃-alkyl, C₂-hydroxyalkyl and (C₁-alkoxy)-(C₂-alkyl)-,
R^{12′} represents, independently of each other, hydrogen, halogen, hydroxy, C₁-C₄-alkyl, C₃-C₆-cycloalkyl, C₁-C₄-alkoxy, C₁-C₄-haloalkoxy, —N(R¹⁸)R¹⁹, —C(O)R¹³, or —C(O)OR¹,
wherein C₁-C₄-alkyl is optionally substituted one, two or three times, independently of each other, with halogen and optionally substituted one time with a substituent selected from hydroxy, C₁-alkoxy, —NH₂, —NH(CH₃), —N(CH₃)₂,
whereby two substituents R^{12′} when they are in adjacent positions of the ring to which they are attached, can be linked to one another in such a way that they jointly form propane-1,3-diyl.

In a further embodiment of the above-mentioned aspects, the invention relates to compounds of formula (I), wherein:

A represents a group selected from:

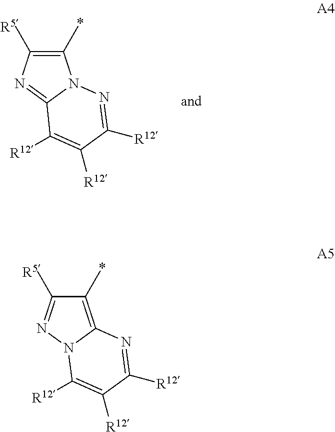

wherein * indicates the point of attachment of said groups with the rest of the molecule,
in which R^{5′} and R^{12′} are as defined herein.

In a further embodiment of the above-mentioned aspects, the invention relates to compounds of formula (I), wherein:

A represents a group selected from:

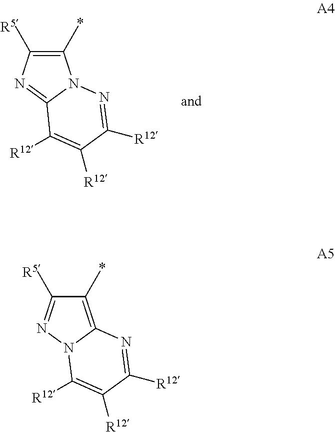

wherein * indicates the point of attachment of said groups with the rest of the molecule,
in which R^{5′} represents, independently of each other, R⁵ or hydrogen, and R^{12′} represents, independently of each other, R¹² or hydrogen, wherein R⁵ and R¹² are as defined herein.

In a further embodiment of the above-mentioned aspects, the invention relates to compounds of formula (I), wherein:

A represents a group selected from:

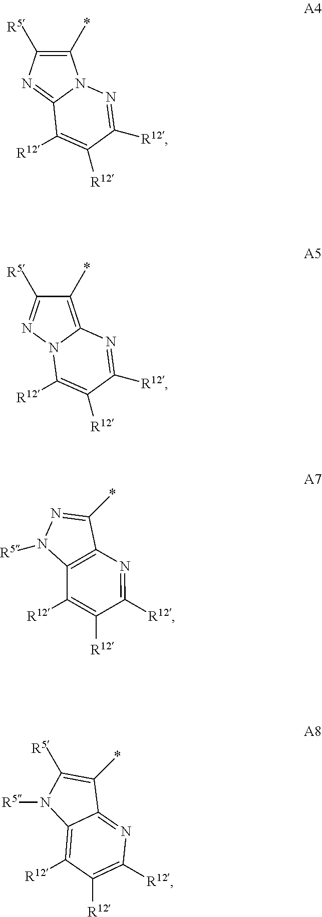

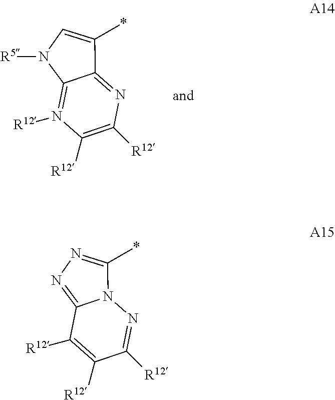

wherein * indicates the point of attachment of said groups with the rest of the molecule,
in which:
R^{5′} represents, independently of each other, R⁵ or hydrogen,
R^{5′} represents, independently of each other, a group selected from:
hydrogen, C₁-C₃-alkyl, C₂-hydroxyalkyl and (C₁-alkoxy)-(C₂-alkyl)-,
and
R^{12′} represents, independently of each other, R¹² or hydrogen,
wherein R⁵ and R¹² are as defined herein.

In a further embodiment of the above-mentioned aspects, the invention relates to compounds of formula (I), wherein:

A represents a group A4:

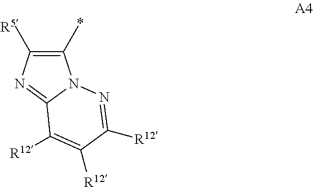

wherein * indicates the point of attachment of said group with the rest of the molecule,
in which R^{5′} and R^{12′} are as defined herein.

In a further embodiment of the above-mentioned aspects, the invention relates to compounds of formula (I), wherein:

A represents a group A4:

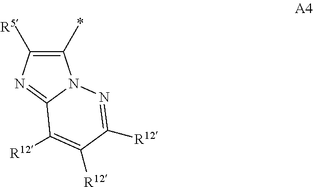

wherein * indicates the point of attachment of said group with the rest of the molecule,
wherein said point of attachment is a carbon atom;
in which R^{5′} represents, independently of each other, R⁵ or hydrogen, and R^{12′} represents, independently of each other, R¹² or hydrogen, wherein R⁵ and R¹² are as defined herein.

In a further embodiment of the above-mentioned aspects, the invention relates to compounds of formula (I), wherein:

A represents a group A5:

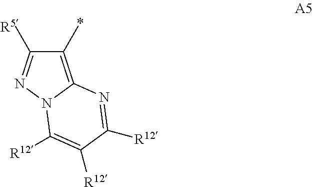

wherein * indicates the point of attachment of said groups with the rest of the molecule,
in which R^{5′} and R^{12′} are as defined herein.

In a further embodiment of the above-mentioned aspects, the invention relates to compounds of formula (I), wherein:

A represents a group selected from:

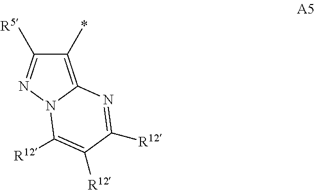

wherein * indicates the point of attachment of said groups with the rest of the molecule,
in which R^{5′} represents, independently of each other, R⁵ or hydrogen, and R^{12′} represents, independently of each other, R¹² or hydrogen, wherein R⁵ and R¹² are as defined herein.

In a further embodiment of the above-mentioned aspects, the invention relates to compounds of formula (I), wherein:

A represents a group selected from:

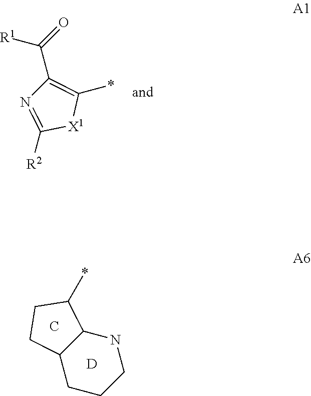

wherein * indicates the point of attachment of said groups with the rest of the molecule, wherein said point of attachment is a carbon atom;

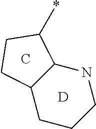

represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R⁵ groups, and ring D represents a 6-membered heteroaryl group which contains one, two or three nitrogen heteroatoms, said ring being optionally substituted with one, two or three R¹² groups,
in which R¹, R², X¹, R⁵ and R¹² are as defined herein.

In a further embodiment of the above-mentioned aspects, the invention relates to compounds of formula (I), wherein:

A represents a group selected from:

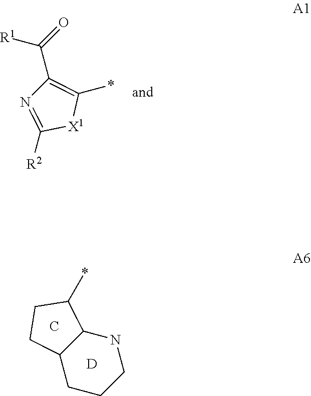

wherein * indicates the point of attachment of said groups with the rest of the molecule,
wherein said point of attachment is a carbon atom;

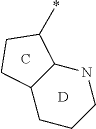

represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R^{5′} groups, and
ring D represents a 6-membered heteroaryl group which contains one, two or three nitrogen heteroatoms, said ring being optionally substituted with one, two or three R^{12′} groups,
in which R¹, R², X¹, R^{5′} and R^{12′} are as defined herein, with the proviso that R^{5′} and R^{12′} are not hydrogen.

In a further embodiment of the above-mentioned aspects, the invention relates to compounds of formula (I), wherein:

X¹ represents NR³ or O.

In a further embodiment of the above-mentioned aspects, the invention relates to compounds of formula (I), wherein:

X¹ represents NR³.

In a further embodiment of the above-mentioned aspects, the invention relates to compounds of formula (I), wherein:

X¹ represents O.

In a further embodiment of the above-mentioned aspects, the invention relates to compounds of formula (I), wherein:

R¹ represents a group selected from:
—OR¹³, and —N(R¹⁴)R¹⁵.

In a further embodiment of the above-mentioned aspects, the invention relates to compounds of formula (I), wherein:

R¹ represents —OR¹³.

In a further embodiment of the above-mentioned aspects, the invention relates to compounds of formula (I), wherein:

R¹ represents —N(R¹⁴)R¹⁵.

In a further embodiment of the above-mentioned aspects, the invention relates to compounds of formula (I), wherein:

R² represents a group selected from:
hydrogen, C₁-C₃-alkyl, and C₃-C₄-cycloalkyl.

In a further embodiment of the above-mentioned aspects, the invention relates to compounds of formula (I), wherein:

R² represents a group selected from:
hydrogen, and C₁-C₃-alkyl.

In a further embodiment of the above-mentioned aspects, the invention relates to compounds of formula (I), wherein:

R² represents a group selected from:
hydrogen, and C₁-C₂-alkyl.

In a further embodiment of the above-mentioned aspects, the invention relates to compounds of formula (I), wherein:

R² represents hydrogen.

In a further embodiment of the above-mentioned aspects, the invention relates to compounds of formula (I), wherein:

R³ represents a hydrogen atom.

In a further embodiment of the above-mentioned aspects, the invention relates to compounds of formula (I), wherein:

R⁴ represents a hydrogen atom.

In a further embodiment of the above-mentioned aspects, the invention relates to compounds of formula (I), wherein:

R⁵ represents, independently of each other, a group selected from:
halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, and —C(O)OR¹³,
wherein C₁-C₄-alkyl is optionally substituted one, two or three times with a group independently selected from halogen, hydroxy, C₁-C₃-alkoxy, —NH₂, —NH(C₁-C₃-alkyl) and —N(C₁-C₃-alkyl)₂.

In a further embodiment of the above-mentioned aspects, the invention relates to compounds of formula (I), wherein:

R⁵ represents, independently of each other, a group selected from:
halogen, hydroxy, C₁-C₃-alkyl, —NH₂,
wherein C₁-C₃-alkyl is optionally substituted one, two or three times with a group independently selected from halogen, hydroxy, C₁-C₃-alkoxy, —NH₂, —NH(C₁-C₃-alkyl) and —N(C₁-C₃-alkyl)₂.

In a further embodiment of the above-mentioned aspects, the invention relates to compounds of formula (I), wherein:

R⁵ represents, independently of each other, a group selected from:
halogen, hydroxy, C₁-alkyl, and —NH₂.

In a further embodiment of the above-mentioned aspects, the invention relates to compounds of formula (I), wherein:

R^{5′} represents, independently of each other, a group selected from:
hydrogen, halogen, hydroxy, C₁-C₃-alkyl and —NH₂,

In a further embodiment of the above-mentioned aspects, the invention relates to compounds of formula (I), wherein:

R^{5′} represents, independently of each other, a group selected from:
hydrogen, hydroxy, C₁-alkyl and —NH₂,

In a further embodiment of the above-mentioned aspects, the invention relates to compounds of formula (I), wherein

R^{5′} represents hydrogen.

In a further embodiment of the above-mentioned aspects, the invention relates to compounds of formula (I), wherein

R^{5′} represents, independently of each other, a group selected from:
hydrogen, C₁-C₃-alkyl, C₂-hydroxyalkyl and (C₁-alkoxy)-(C₂-alkyl)-.

In a further embodiment of the above-mentioned aspects, the invention relates to compounds of formula (I), wherein

R^{5′} represents, independently of each other, a group selected from:
hydrogen, C₁-alkyl, C₂-hydroxyalkyl and (C₁-alkoxy)-(C₂-alkyl)-.

In a further embodiment of the above-mentioned aspects, the invention relates to compounds of formula (I), wherein

R^{5′} represents, independently of each other, a group selected from:
hydrogen, and C₁-alkyl.

In a further embodiment of the above-mentioned aspects, the invention relates to compounds of formula (I), wherein:

R⁶ represents hydrogen, halogen, hydroxy, C₁-C₃-alkyl or C₁-C₃-alkoxy;
R⁷ represents hydrogen; or
R⁶, R⁷ represent, independently of each other, halogen.

In a further embodiment of the above-mentioned aspects, the invention relates to compounds of formula (I), wherein:

R⁶ represents hydrogen, halogen, hydroxy, C₁-C₃-alkyl or C₁-C₃-alkoxy;

In a further embodiment of the above-mentioned aspects, the invention relates to compounds of formula (I), wherein:

R⁶ represents hydrogen, halogen, hydroxy, C₁-C₃-alkyl or C₁-C₃-alkoxy;
R⁷ represents hydrogen.

In a further embodiment of the above-mentioned aspects, the invention relates to compounds of formula (I), wherein:

R⁷ represents hydrogen.

In a further embodiment of the above-mentioned aspects, the invention relates to compounds of formula (I), wherein:

R⁶, R⁷ represent, independently of each other, halogen, preferably fluorine.

In a further embodiment of the above-mentioned aspects, the invention relates to compounds of formula (I), wherein:

R⁶ represents hydrogen, halogen, hydroxy, C₁-alkyl or C₁-alkoxy;
R⁷ represents hydrogen.

In a further embodiment of the above-mentioned aspects, the invention relates to compounds of formula (I), wherein:

R⁶ represents hydrogen, halogen, C-alkyl or C₁-alkoxy;
R⁷ represents hydrogen.

In a further embodiment of the above-mentioned aspects, the invention relates to compounds of formula (I), wherein:

in embodiment a), R⁸, R⁹ and R¹⁰ represent:
R⁸ represents hydrogen,
R⁹ represents hydrogen, fluorine or C₁-alkyl; or
R⁸ and R⁹ together represent a group:

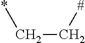

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, or C₁-C₃-haloalkyl.

In a further embodiment of the above-mentioned aspects, the invention relates to compounds of formula (I), wherein:

in embodiment a), R⁸, R⁹ and R¹⁰ represent:
R⁸ represents hydrogen,
R⁹ represents hydrogen, fluorine or C₁-alkyl; or
R⁸ and R⁹ together represent a group:

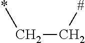

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, or C₂-C₃-hydroxyalkyl.

In a further embodiment of the above-mentioned aspects, the invention relates to compounds of formula (I), wherein:

in embodiment a), R⁸, R⁹ and R¹⁰ represent:
R⁸ represents hydrogen, or C₁-C₃-alkyl,
R⁹ represents hydrogen, halogen, C₁-C₃-alkoxy, or C₁-C₃-alkyl optionally substituted with one, two or three groups independently selected from hydroxy, halogen and C₃-C₄-cycloalkyl; or
R⁸ and R⁹ together represent a group:

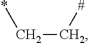

wherein * indicates the point of attachment of said group to the rest of the molecule at R⁸,
and ^{#} indicates the point of attachment of said group to the rest of the molecule at R⁹,
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-(C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-.

In a further embodiment of the above-mentioned aspects, the invention relates to compounds of formula (I), wherein:

in embodiment a), R⁸, R⁹ and R¹⁰ represent:
R⁸ represents hydrogen,
R⁹ represents hydrogen, or C₁-alkyl; or
R⁸ and R⁹ together represent a group:

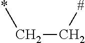

wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, or C₁-C₃-haloalkyl.

In a further embodiment of the above-mentioned aspects, the invention relates to compounds of formula (I), wherein:

in embodiment a), R⁸, R⁹ represent:
R⁸ represents hydrogen, or C₁-C₃-alkyl,
R⁹ represents hydrogen, halogen, C₁-C₃-alkoxy, or C₁-C₃-alkyl optionally substituted with one, two or three groups independently selected from hydroxy, halogen and C₃-C₄-cycloalkyl.

In a further embodiment of the above-mentioned aspects, the invention relates to compounds of formula (I), wherein:

in embodiment a), R⁸, R⁹ represent:
R⁸ represents hydrogen,
R⁹ represents hydrogen.

In a further embodiment of the above-mentioned aspects, the invention relates to compounds of formula (I), wherein:

in embodiment a), R⁸, R⁹ represent:
R⁸ and R⁹ together represent a group:

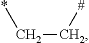

wherein * indicates the point of attachment of said group to the rest of the molecule at R⁸,
and ^{#} indicates the point of attachment of said group to the rest of the molecule at R⁹.

In a further embodiment of the above-mentioned aspects, the invention relates to compounds of formula (I), wherein:

in embodiment a), R⁸ represents:
R⁸ represents hydrogen, or C₁-C₃-alkyl, preferably hydrogen.

In a further embodiment of the above-mentioned aspects, the invention relates to compounds of formula (I), wherein:

in embodiment a), R⁹ represents:
R⁹ represents hydrogen, halogen, C₁-C₃-alkoxy, or C₁-C₃-alkyl optionally substituted with one, two or three groups independently selected from hydroxy, halogen and C₃-C₄-cycloalkyl.

In a further embodiment of the above-mentioned aspects, the invention relates to compounds of formula (I), wherein:

in embodiment a), R⁹ represents:
R⁹ represents hydrogen, halogen, C₁-alkoxy or C₁-C₃-alkyl optionally substituted with one, two or three groups independently selected from hydroxy and halogen.

In a further embodiment of the above-mentioned aspects, the invention relates to compounds of formula (I), wherein:

in embodiment a), R⁹ represents:
R⁹ represents hydrogen, halogen, or C₁-C₃-alkyl.

In a further embodiment of the above-mentioned aspects, the invention relates to compounds of formula (I), wherein:

in embodiment a), R⁹ represents:
R⁹ represents hydrogen, or C₁-alkyl, preferably hydrogen.

In a further embodiment of all aspects of the invention described herein, the invention relates to compounds of formula (I), wherein:

in embodiment a), R¹⁰ represents:
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-.

In a further embodiment of all aspects of the invention described herein, the invention relates to compounds of formula (I), wherein:

in embodiment a), R¹⁰ represents:
R¹⁰ represents hydrogen, C₁-C₃-alkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, or C₁-C₃-haloalkyl.

In a further embodiment of all aspects of the invention described herein, the invention relates to compounds of formula (I), wherein:

in embodiment a), R¹⁰ represents:
R¹⁰ represents hydrogen, C₁-C₃-alkyl, (C₃-C₄-cycloalkyl)-(C₁-C₃-alkyl)-, or C₂-C₃-hydroxyalkyl.

In a preferred embodiment of all aspects of the invention described herein, the invention relates to compounds of formula (I), wherein:

in embodiment b), R⁸, R⁹ and R¹⁰ represent:
R⁸ represents hydrogen, or C₁-C₃-alkyl, and
R⁹ and R¹⁰ together represent a group selected from:

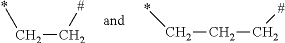

wherein said groups are optionally substituted with one or two groups, which are independently of each other selected from: halogen, C₁-C₃-alkyl and C₁-C₃-alkoxy, hydroxy, C₁-C₃-haloalkyl, C₁-C₃-hydroxyalkyl,
wherein * indicates the point of attachment of said group to the rest of the molecule at R⁹,
and ^{#} indicates the point of attachment of said group to the rest of the molecule at R¹⁰.

In a further embodiment of the above-mentioned aspects, the invention relates to compounds of formula (I), wherein:

in embodiment b), R⁸, R⁹ and R¹⁰ represent:
R⁸ represents hydrogen,
R⁹ and R¹⁰ together represent a group selected from:

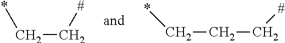

wherein * indicates the point of attachment of said group to the rest of the molecule at R⁹,
and ^{#} indicates the point of attachment of said group to the rest of the molecule at R¹.

In a further embodiment of the above-mentioned aspects, the invention relates to compounds of formula (I), wherein:

in embodiment b), R⁸ represents:
R⁸ represents hydrogen, or C₁-C₃-alkyl, preferably hydrogen.

In a further embodiment of the above-mentioned aspects, the invention relates to compounds of formula (I), wherein:

in embodiment b), R⁹ and R¹⁰ represent:
R⁹ and R¹⁰ together represent a group selected from:

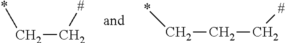

wherein said groups are optionally substituted with one or two groups, which are independently of each other selected from:
halogen, C₁-C₃-alkyl, C₁-C₃-alkoxy, hydroxy, C₁-C₃-haloalkyl, and C₁-C₃-hydroxyalkyl,
wherein * indicates the point of attachment of said group to the rest of the molecule at R⁹,
and ^{#} indicates the point of attachment of said group to the rest of the molecule at R¹⁰.

In a further embodiment of the above-mentioned aspects, the invention relates to compounds of formula (I), wherein:

in embodiment b), R⁹ and R¹⁰ represent:
R⁹ and R¹⁰ together represent a group selected from:

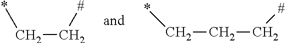

wherein said groups are optionally substituted with one or two groups, which are independently of each other selected from:
C₁-alkyl and hydroxy,
wherein * indicates the point of attachment of said group to the rest of the molecule at R⁹,
and ^{#} indicates the point of attachment of said group to the rest of the molecule at R¹⁰.

In a further embodiment of the above-mentioned aspects, the invention relates to compounds of formula (I), wherein:

in embodiment b), R⁹ and R¹⁰ represent:
R⁹ and R¹⁰ together represent a group selected from:

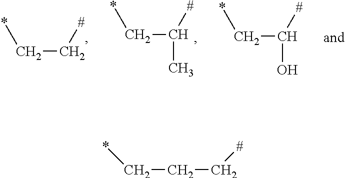

wherein * indicates the point of attachment of said group to the rest of the molecule at R⁹,
and ^{#} indicates the point of attachment of said group to the rest of the molecule at R¹⁰.

In a further embodiment of the above-mentioned aspects, the invention relates to compounds of formula (I), wherein:

in embodiment b), R⁹ and R¹⁰ represent:
R⁹ and R¹⁰ together represent a group selected from:

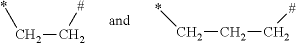

wherein * indicates the point of attachment of said group to the rest of the molecule at R⁹,
and ^{#} indicates the point of attachment of said group to the rest of the molecule at R¹⁰.

In a further embodiment of the above-mentioned aspects, the invention relates to compounds of formula (I), wherein:

R¹¹ represents a group selected from:
aryl, and heteroaryl,
   wherein aryl and heteroaryl groups are optionally substituted with one, two, three or four groups, which are independently of each other selected from:
   C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein:

R¹¹ represents a group selected from:
aryl, and heteroaryl,
   wherein aryl and heteroaryl groups are optionally substituted with one, two, three or four groups, which are independently of each other selected from:
   C₁-C₄-alkyl, C₁-C₃-alkoxy, C₁-C₃-hydroxyalkyl, C₃-C₄-cycloalkyl, C₃-C₄-cycloalkoxy, C₁-C₃-haloalkyl, C₁-C₃-haloalkoxy, halogen, cyano, hydroxy, R¹⁴(R¹⁵)N—(C₁-C₃-alkyl)-, and R¹⁴(R¹⁵)N—(C₂-C₃-alkoxy)-,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein

R¹¹ represents a group selected from:
aryl, and heteroaryl,
   wherein aryl and heteroaryl groups are optionally substituted with one, two, three or four groups, which are independently of each other selected from:
   C₁-C₄-alkyl, C₁-C₃-alkoxy, C₁-C₃-hydroxyalkyl, C₁-C₃-haloalkyl, halogen and, cyano.

In a further embodiment of the above-mentioned aspects, the invention relates to compounds of formula (I), wherein

R¹¹ represents a group selected from:
phenyl, pyridinyl, pyrimidinyl and 1,2-thiazolyl,
   wherein said groups are optionally substituted with one, two, three or four groups, which are independently of each other selected from:
   C₁-C₄-alkyl, C₁-C₃-alkoxy, C₁-C₃-hydroxyalkyl, C₃-C₄-cycloalkyl, C₃-C₄-cycloalkoxy, C₁-C₃-haloalkyl, C₁-C₃-haloalkoxy, halogen, cyano, hydroxy, R¹⁴(R¹⁵)N—(C₁-C₃-alkyl)-, and R¹⁴(R¹⁵)N—(C₂-C₃-alkoxy)-,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein:

R¹¹ represents a group selected from:
phenyl, pyridinyl, pyrimidinyl and 1,2-thiazolyl,
   wherein said groups are optionally substituted with one, two, three or four groups, which are independently of each other selected from:
   C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²)R²², —N(R¹⁴)R⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
   whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein

R¹¹ represents a group selected from:
phenyl, pyridinyl, pyrimidinyl and 1,2-thiazolyl,
   wherein said groups are optionally substituted with one, two, three or four groups, which are independently of each other selected from:
   C₁-C₄-alkyl, C₁-C₃-alkoxy, C₁-C₃-hydroxyalkyl, C₁-C₃-haloalkyl, halogen and, cyano.

In a further embodiment of the above-mentioned aspects, the invention relates to compounds of formula (I), wherein:

R¹¹ represents aryl, preferably phenyl,
wherein aryl is optionally substituted with one, two, three or four groups, which are independently of each other selected from:
C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R⁷, —C(═O)OH, —C(═O)OR¹³, and —C(═O)N(R¹⁶)₂,
whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein:

R¹¹ represents aryl, preferably phenyl,
wherein aryl is optionally substituted with one, two, three or four groups, which are independently of each other selected from:
C₁-C₄-alkyl, C₁-C₃-alkoxy, C₁-C₃-hydroxyalkyl, C₃-C₄-cycloalkyl, C₃-C₄-cycloalkoxy, C₁-C₃-haloalkyl, C₁-C₃-haloalkoxy, halogen, cyano, hydroxy, R¹⁴(R¹⁵)N—(C₁-C₃-alkyl)-, and R¹⁴(R¹⁵)N—(C₂-C₃-alkoxy)-,
whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein

R¹¹ represents aryl, preferably phenyl,
wherein aryl is optionally substituted with one, two or three groups, which are independently of each other selected from:
C₁-C₄-alkyl, C₁-C₃-alkoxy, C₁-C₃-hydroxyalkyl, C₁-C₃-haloalkyl, halogen and, cyano.

In a further embodiment of the above-mentioned aspects, the invention relates to compounds of formula (I), wherein

R¹¹ represents heteroaryl, preferably pyridinyl, pyrimidinyl or 1,2-thiazolyl,
wherein said groups are optionally substituted with one, two, or three groups, which are independently of each other selected from:
C₁-C₄-alkyl, C₁-C₃-alkoxy, C₁-C₃-hydroxyalkyl, C₃-C₄-cycloalkyl, C₃-C₄-cycloalkoxy, C₁-C₃-haloalkyl, C₁-C₃-haloalkoxy, halogen, cyano, hydroxy, R¹⁴(R¹⁵)N—(C₁-C₃-alkyl)-, and R¹⁴(R¹⁵)N—(C₂-C₃-alkoxy)-.

In a further embodiment of the above-mentioned aspects, the invention relates to compounds of formula (I), wherein:

R¹¹ represents heteroaryl, preferably pyridinyl, pyrimidinyl or 1,2-thiazolyl,
wherein said groups are optionally substituted with one, two, or three groups, which are independently of each other selected from:
   C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂.

In a further embodiment of the above-mentioned aspects, the invention relates to compounds of formula (I), wherein

R¹¹ represents heteroaryl, preferably pyridinyl, pyrimidinyl or 1,2-thiazolyl,
wherein said groups are optionally substituted with one, two, three or four groups, which are independently of each other selected from:
C₁-C₄-alkyl, C₁-C₃-alkoxy, C₁-C₃-hydroxyalkyl, C₁-C₃-haloalkyl, halogen and, cyano.

In a further embodiment of the above-mentioned aspects, the invention relates to compounds of formula (I), wherein

R¹¹ represents phenyl, pyridinyl, or pyrimidinyl,
wherein said groups are optionally substituted with one, two or, three groups, which are independently of each other selected from:
C₁-alkyl, C₁-alkoxy, C₃-hydroxyalkyl, C₁-haloalkyl, cyano and halogen, independently selected from fluorine, chlorine and bromine.

In a further embodiment of the above-mentioned aspects, the invention relates to compounds of formula (I), wherein

R¹² represents, independently of each other, halogen, hydroxy, C₁-C₆-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
wherein C₁-C₆-alkyl is optionally substituted one, two or three times with a substituent independently selected from halogen, hydroxy, C₁-C₃-alkoxy, C₁-C₃-haloalkoxy, and —N(R¹⁸)R¹⁹;
whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl.

In a further embodiment of the above-mentioned aspects, the invention relates to compounds of formula (I), wherein

R¹² represents, independently of each other, halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, or —C(O)OR¹³,
wherein C₁-C₄-alkyl is optionally substituted one, two or three times with halogen and optionally substituted one time with a substituent independently selected from hydroxy, C₁-C₃-alkoxy, C₁-C₃-haloalkoxy, —N(R¹⁸)R¹⁹;
whereby two substituents R¹² when they are in adjacent positions of the ring to which they are attached, can be linked to one another in such a way that they jointly form propane-1,3-diyl.

In a further embodiment of the above-mentioned aspects, the invention relates to compounds of formula (I), wherein

R^{12′} represents, independently of each other, hydrogen, halogen, hydroxy, C₁-C₄-alkyl, C₃-C₄-cycloalkyl, C₁-alkoxy, —N(R¹⁸)R¹⁹, —C(O)R¹³, or —C(O)OR¹³,
wherein C₁-C₄-alkyl is optionally substituted one, two or three times, independently of each other, with halogen and optionally substituted one time with a substituent selected from hydroxy, C₁-C₃-alkoxy, —NH₂, —NH(CH₃), —N(CH₃)₂,
whereby two substituents R^{12′} when they are in adjacent positions of the ring to which they are attached, can be linked to one another in such a way that they jointly form propane-1,3-diyl.

In a further embodiment of the above-mentioned aspects, the invention relates to compounds of formula (I), wherein

R¹³ represents a group selected from:
C₁-C₆-alkyl, C₃-C₆-cycloalkyl, C₂-C₆-hydroxyalkyl-, and (C₁-C₃-alkoxy)-(C₂-C₆-alkyl)-.

In a further embodiment of the above-mentioned aspects, the invention relates to compounds of formula (I), wherein

R¹³ represents a group selected from:
C₁-C₃-alkyl, C₃-C₄-cycloalkyl, C₂-C₃-hydroxyalkyl-, and (C₁-alkoxy)-(C₂-C₃-alkyl)-.

In a further embodiment of the above-mentioned aspects, the invention relates to compounds of formula (I), wherein

R¹³ represents C₁-C₂-alkyl.

In a further embodiment of the above-mentioned aspects, the invention relates to compounds of formula (I), wherein:

R¹⁴ and R¹⁵ are, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, —NH(C₁-C₃-alkyl), —N(C₁-C₃-alkyl)₂, hydroxy, a halogen atom, and cyano,
   and,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
      or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
      said azetidine and pyrrolidine being optionally substituted one time with C₁-C₃-alkyl or C₁-C₃-haloalkyl,
      or,
      R¹⁴ and R¹⁵ together with the nitrogen atom to which they are attached form a group selected from:

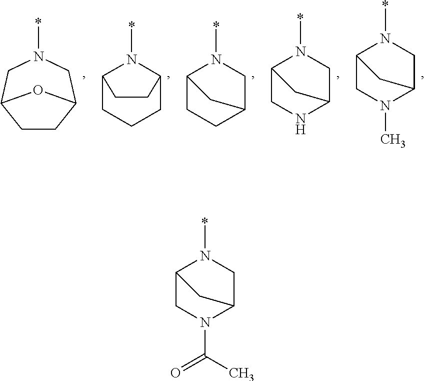

wherein * indicates the point of attachment of said group with the rest of the molecule,

In a further embodiment of the above-mentioned aspects, the invention relates to compounds of formula (I), wherein

R¹⁴ and R¹⁵ are, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, —NH(C₁-C₃-alkyl), —N(C₁-C₃-alkyl)₂, hydroxy, a halogen atom, and cyano,
   and,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂.

In a further embodiment of the above-mentioned aspects, the invention relates to compounds of formula (I), wherein

R¹⁴ is, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, —NH(C₁-C₃-alkyl), —N(C₁-C₃-alkyl)₂, hydroxy, a halogen atom, and cyano,
   and,
   wherein aryl and heteroaryl groups are optionally substituted with one or two substituents, which are independently of each other selected from:
   C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
      R¹⁵ is, independently of each other, selected from:
hydrogen, C₁-C₃-alkyl, C₁-C₃-haloalkyl and C₃-C₄-cycloalkyl, preferably hydrogen.

In a further embodiment of the above-mentioned aspects, the invention relates to compounds of formula (I), wherein:
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
      said azetidine and pyrrolidine being optionally substituted one time with C₁-C₃-alkyl or C₁-C₃-haloalkyl.

In a further embodiment of the above-mentioned aspects, the invention relates to compounds of formula (I), wherein

R¹⁴ and R¹⁵ together with the nitrogen atom to which they are attached form a group selected from:

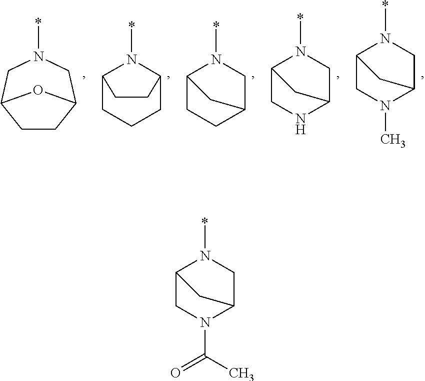

wherein * indicates the point of attachment of said group with the rest of the molecule.

In a further embodiment of the above-mentioned aspects, the invention relates to compounds of formula (I), wherein:

R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl H₂N—(C₂-C₃-alkyl)-, (C₁-C₃-alkyl)N(H)(C₂-C₃-alkyl)-, (C₁-C₃-alkyl)₂N(C₂-C₃-alkyl)-, 4- to 6-membered heterocycloalkyl, (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom.

In a further embodiment of the above-mentioned aspects, the invention relates to compounds of formula (I), wherein:

R¹⁴ is independently of each other, selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl H₂N—(C₂-C₃-alkyl)-, (C₁-C₃-alkyl)N(H)(C₂-C₃-alkyl)-, (C₁-C₃-alkyl)₂N(C₂-C₃-alkyl)-, 4- to 6-membered heterocycloalkyl, (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
      R¹⁵ is, independently of each other, selected from:
hydrogen, C₁-C₃-alkyl, C₁-C₃-haloalkyl and C₃-C₄-cycloalkyl, preferably hydrogen.

In a further embodiment of the above-mentioned aspects, the invention relates to compounds of formula (I), wherein:
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
      C₁-alkyl, C-haloalkyl, C₁-alkoxy, C₁-haloalkoxy, C₃-C₄-cycloalkyl, C₃-C₄-cycloalkoxy, —N(CH₃)₂, —N(H)₂, —N(CH₃)H, hydroxy, and a halogen atom,
      whereby when two substituents are attached to the same ring carbon atom, together with the carbon atom to which they are attached, can be linked to one another in such a way that they jointly form a azetidine, or oxetane;
      said azetidine being optionally substituted one time with C₁-alkyl or C₁-haloalkyl.

In a further embodiment of the above-mentioned aspects, the invention relates to compounds of formula (I), wherein:

R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom.

In a further embodiment of the above-mentioned aspects, the invention relates to compounds of formula (I), wherein:

R¹⁴ is independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom.
      R¹⁵ is, independently of each other, selected from:
hydrogen, C₁-C₃-alkyl, C₁-C₃-haloalkyl and C₃-C₄-cycloalkyl, preferably hydrogen.

In a further embodiment of the above-mentioned aspects, the invention relates to compounds of formula (I), wherein:
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂,
   said 4- to 7-membered heterocycloalkyl group being optionally substituted with one or two groups, which are independently of each other selected from:
      C₁-alkyl, C₁-alkoxy, —N(CH₃)₂, and a halogen atom,
      whereby when two substituents are attached to the same ring carbon atom, together with the carbon atom to which they are attached, can be linked to one another in such a way that they jointly form a azetidine, thietane 1,1-dioxide, or oxetane;
      said azetidine being optionally substituted one time with C₁-alkyl or C₁-haloalkyl.

In a further embodiment of the above-mentioned aspects, the invention relates to compounds of formula (I), wherein:

R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, 4- to 6-membered heterocycloalkyl and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom.

In a further embodiment of the above-mentioned aspects, the invention relates to compounds of formula (I), wherein:

R¹⁴ is independently of each other, selected from:
hydrogen, C₁-C₃-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, 4- to 6-membered heterocycloalkyl and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom.
      R¹⁵ is, independently of each other, selected from:
hydrogen, C₁-C₃-alkyl, C₁-C₃-haloalkyl and C₃-C₄-cycloalkyl, preferably hydrogen.

In a further embodiment of the above-mentioned aspects, the invention relates to compounds of formula (I), wherein:

R¹⁶ represents, independently of each other, hydrogen, or C₁-C₃-alkyl.

In a further embodiment of the above-mentioned aspects, the invention relates to compounds of formula (I), wherein:

In a further embodiment of the above-mentioned aspects, the invention relates to compounds of formula (I), wherein:

R¹⁷ represents hydrogen, C₁-C₆-alkyl, C₁-C₆-hydroxyalkyl, C₃-C₆-cycloalkyl, C₁-C₆-haloalkyl, (C₁-C₃-alkoxy)-(C₁-C₆-alkyl)-, aryl, or heteroaryl,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy.

In a further embodiment of the above-mentioned aspects, the invention relates to compounds of formula (I), wherein:

R¹⁷ represents hydrogen, C₁-C₃-alkyl, C₁-C₃-hydroxyalkyl, C₃-C₄-cycloalkyl, C₁-C₃-haloalkyl, (C₁-C₃-alkoxy)-(C₁-C₃-alkyl)-, or phenyl.

In a further embodiment of the above-mentioned aspects, the invention relates to compounds of formula (I), wherein:

R¹⁸ and R¹⁹ are, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, and heteroaryl,
   wherein 4- to 6-membered heterocycloalkyl groups are optionally substituted with one two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
   and,
   wherein aryl and heteroaryl groups are optionally substituted with one or two substituents, which are independently of each other selected from:
   C₁-C₃-alkyl, halogen, cyano, or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group,
      said azetidine and pyrrolidine being optionally substituted one time with C₁-C₃-alkyl or C₁-C₃-haloalkyl.

In a further embodiment of the above-mentioned aspects, the invention relates to compounds of formula (I), wherein:

R¹⁸ and R¹⁹ are, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, and heteroaryl,
   wherein 4- to 6-membered heterocycloalkyl groups are optionally substituted with one two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
   and,
   wherein aryl and heteroaryl groups are optionally substituted with one or two substituents, which are independently of each other selected from:
   C₁-C₃-alkyl, halogen, cyano.

In a further embodiment of the above-mentioned aspects, the invention relates to compounds of formula (I), wherein:

R¹⁸ is, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, and heteroaryl,
   wherein 4- to 6-membered heterocycloalkyl groups are optionally substituted with one two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
   and,
   wherein aryl and heteroaryl groups are optionally substituted with one or two substituents, which are independently of each other selected from:
   C₁-C₃-alkyl, halogen, cyano,
      R¹⁹ is, independently of each other, selected from hydrogen, C₁-C₄-alkyl, C₁-C₃-haloalkyl, and C₃-C₄-cycloalkyl, preferably hydrogen.

In a further embodiment of the above-mentioned aspects, the invention relates to compounds of formula (I), wherein:
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
      whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group,
      said azetidine and pyrrolidine being optionally substituted one time with C₁-C₃-alkyl or C₁-C₃-haloalkyl.

In a further embodiment of the above-mentioned aspects, the invention relates to compounds of formula (I), wherein:

R¹⁸ and R¹⁹ are, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano.

In a further embodiment of the above-mentioned aspects, the invention relates to compounds of formula (I), wherein:

R¹⁸ is, independently of each other, selected from:
hydrogen, C₁-C₆-alkyl, C₃-C₆-cycloalkyl, (C₃-C₆-cycloalkyl)-(C₁-C₆-alkyl)-C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, and (4- to 6-membered heterocycloalkyl)-(C₁-C₆-alkyl)-,
   wherein 4- to 6-membered heterocycloalkyl groups are optionally substituted with one, two, three or four substituents, which are independently of each other selected from:
   C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano.

R¹⁹ is, independently of each other, selected from hydrogen, C₁-C₄-alkyl, C₁-C₃-haloalkyl, and C₃-C₄-cycloalkyl, preferably hydrogen.

In a further embodiment of the above-mentioned aspects, the invention relates to compounds of formula (I), wherein:
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O),
   said 4- to 7-membered heterocycloalkyl group being optionally substituted with one, two, three or four groups, which are independently of each other selected from:
      C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano.

In a further embodiment of the above-mentioned aspects, the invention relates to compounds of formula (I), wherein:

R¹⁸ and R¹⁹ are, independently of each other, selected from:
hydrogen, C₁-C₄-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₃-alkyl)-, C₁-C₃-haloalkyl, H₂N—(C₂-C₃-alkyl)-, (C₁-alkyl)N(H)(C₂-C₃-alkyl)-, (C₁-alkyl)₂N(C₂-C₃-alkyl)-, HOC(═O)—(C₁-C₃-alkyl)-, R¹³OC(═O)—(C₁-C₃-alkyl)-, 4- to 6-membered heterocycloalkyl, (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, —NH₂, hydroxy, and a halogen atom.

In a further embodiment of the above-mentioned aspects, the invention relates to compounds of formula (I), wherein:

R¹⁸ is, independently of each other, selected from:
hydrogen, C₁-C₄-alkyl, C₃-C₄-cycloalkyl, (C₃-C₄-cycloalkyl)-(C₁-C₃-alkyl)-C₂-C₄-hydroxyalkyl, (C₁-alkoxy)-(C₂-C₃-alkyl)-, C₁-C₃-haloalkyl, H₂N—(C₂-C₃-alkyl)-, (C₁-alkyl)N(H)(C₂-C₃-alkyl)-, (C₁-alkyl)₂N(C₂-C₃-alkyl)-, HOC(═O)—(C₁-C₃-alkyl)-, R¹³OC(═O)—(C₁-C₃-alkyl)-, 4- to 6-membered heterocycloalkyl, and (4- to 6-membered heterocycloalkyl)-(C₁-C₃-alkyl)-,
   wherein 4- to 6-membered heterocycloalkyl groups are optionally substituted with one or two substituents, which are independently of each other selected from:
   C₁-alkyl, C₁-haloalkyl, C₁-alkoxy, C₁-haloalkoxy, —NH₂, hydroxy, and a halogen atom.
      R¹⁹ is, independently of each other, selected from hydrogen, C₁-C₄-alkyl, C₁-C₃-haloalkyl, and C₃-C₄-cycloalkyl, preferably hydrogen.

In a further embodiment of the above-mentioned aspects, the invention relates to compounds of formula (I), wherein:
R¹⁸ and R¹⁹ together with the nitrogen atom to which they are attached form a 5-6-membered heterocycloalkyl group, in which one carbon atom is optionally replaced by a further heteroatom-containing group selected from NR²⁰, 0,
   said 5-6-membered heterocycloalkyl group being optionally substituted with one or two groups, which are independently of each other selected from:
      C₁-alkyl, C₁-haloalkyl, C₁-alkoxy, C₁-haloalkoxy, —NH₂, —N(CH₃)₂, N(CH₃)H, hydroxy, and a halogen atom.

In a further embodiment of the above-mentioned aspects, the invention relates to compounds of formula (I), wherein:

R¹⁸ and R¹⁹ are, independently of each other, selected from:
hydrogen and C₁-alkyl.

In a further embodiment of the above-mentioned aspects, the invention relates to compounds of formula (I), wherein:
R¹⁸ and R¹⁹ together with the nitrogen atom to which they are attached form a 5-6-membered heterocycloalkyl group, in which one carbon atom is optionally replaced by a further heteroatom-containing group selected from NR²⁰, and O.

In a further embodiment of the above-mentioned aspects, the invention relates to compounds of formula (I), wherein:

R²⁰ represents, independently of each other, a group selected from:
hydrogen, C₁-C₃-alkyl, C₁-C₃-haloalkyl, and C₃-C₄-cycloalkyl.

In a further embodiment of the above-mentioned aspects, the invention relates to compounds of formula (I), wherein:

R²⁰ represents, independently of each other, a group selected from:
hydrogen, C₁-alkyl, and C₁-C₂-haloalkyl.

In a further embodiment of the above-mentioned aspects, the invention relates to compounds of formula (I), wherein:

R²¹ represents hydrogen, cyano, (C₁-C₃-alkyl)-C(═O)—, or (C₁-C₃-haloalkyl)-C(═O)—.

In a further embodiment of the above-mentioned aspects, the invention relates to compounds of formula (I), wherein:

In a further embodiment of the above-mentioned aspects, the invention relates to compounds of formula (I), wherein:

R²² represents C₁-C₄-alkyl, or C₃-C₄-cycloalkyl.

In a further embodiment of the above-mentioned aspects, the invention relates to compounds of formula (I), according to any of the above-mentioned embodiments, in the form of or a stereoisomer, a tautomer, an N-oxide, a hydrate, a solvate, or a salt thereof, or a mixture of same.

It is to be understood that the present invention relates to any sub-combination within any embodiment or aspect of the present invention of compounds of general formula (I), supra.

More particularly still, the present invention covers compounds of general formula (I) which are disclosed in the Example section of this text, infra.

In accordance with another aspect, the present invention covers methods of preparing compounds of the present invention, said methods comprising the steps as described in the Experimental Section herein.

Another aspect of the invention is intermediate (C):

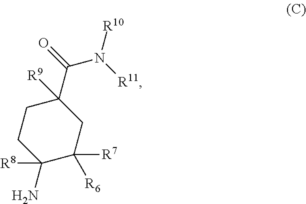

in which R⁶, R⁷, R⁸, R⁹, R¹⁰ and R¹¹ are as defined for the compound of general formula (I) supra or in the examples below.

Another aspect of the invention is intermediate (C-b):

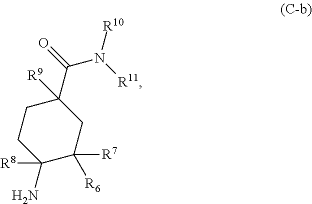

in which R⁶, R⁷, and R¹ are as defined for the compound of general formula (I) supra or in the examples below, and R⁸, R⁹ and R¹⁰ are as defined in embodiment b) for the compound of general formula (I) supra or in the examples below.

Another aspect of the invention is intermediate (3-3):

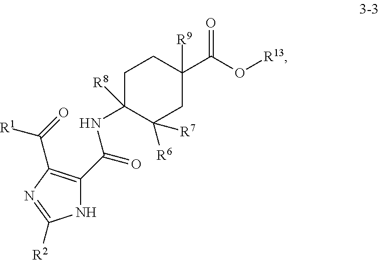

in which R¹, R², R⁶, R⁷, R⁸, R⁹ and R¹³ are as defined for the compound of general formula (I) supra or in the examples below.

Another aspect of the invention is intermediate (3-4):

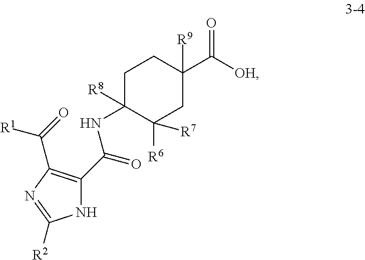

in which R¹, R², R⁶, R⁷, R⁸, and R⁹ are as defined for the compound of general formula (I) supra or in the examples below.

Another aspect of the invention is intermediate (3-8):

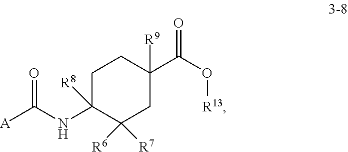

in which A represents A3, A4, A5 A6, A7, A8, A9, A10, A11, A12, A13, A14 or A15, and R⁶, R⁷, R⁸, R⁹, R¹³, A3, A4, A5, A6, A7, A8, A9, A10, A11, A12, A13, A14 and A15 are as defined for the compound of general formula (I) supra or in the examples below.

Another aspect of the invention is intermediate (3-9):

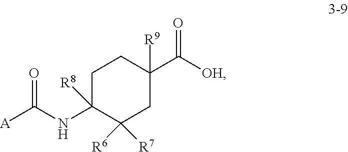

in which A represents A3, A4, A5, A6 A7, A8, A9, A10, A11, A12, A13, A14 or A15, and R⁶, R⁷, R⁸, R⁹, A3, A4, A5, A6, A7, A8, A9, A10, A11, A12, A13, A14 and A15 are as defined for the compound of general formula (I) supra or in the examples below.

Another aspect of the invention is intermediate (3-12):

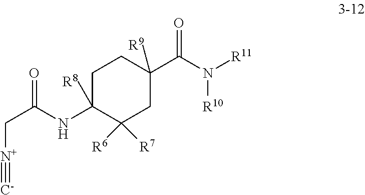

in which R⁶, R⁷, R⁸, R⁹, R¹⁰ and R¹¹ are as defined for the compound of general formula (I) supra or in the examples below.

Another aspect of the invention is intermediate (1-19 (C)):

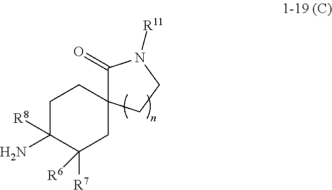

in which n is 1 or 2 and R⁶, R⁷, R⁸, and R¹¹ are as defined for the compound of general formula (I) supra or in the examples below.

Another aspect of the invention is intermediate (1-18):

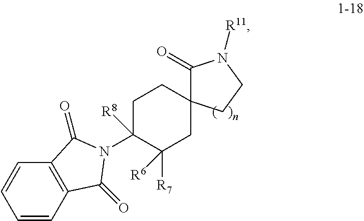

in which n is 1 or 2 and R⁶, R⁷, R⁸, and R¹¹ are as defined for the compound of general formula (I) supra or in the examples below.

Another aspect of the invention is intermediate (3-7):

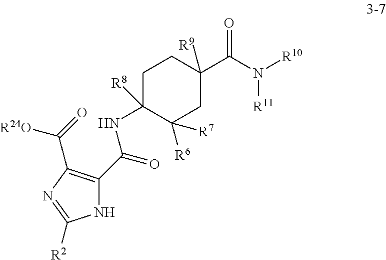

in which R²⁴ represents phenyl and R², R⁶, R⁷, R⁸, R⁹, R¹⁰, and R¹¹ are as defined for the compound of general formula (I) supra or in the examples below.

Another aspect of the invention is intermediate (1-33):

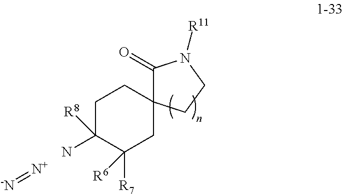

in which n is 1 or 2, and R⁶, R⁷, R⁸, and R¹¹ are as defined for the compound of general formula (I) supra or in the examples below.

Another aspect of the invention is intermediate (I-PG):

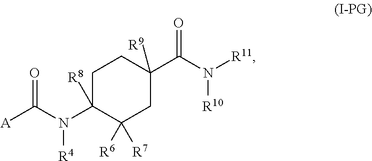

in which, A, X¹, R¹, R², R³, R⁴, R⁵, R⁶, R⁷, R⁸, R⁹, R¹⁰, R¹¹, R¹², R¹³, R¹⁶, R¹⁷, R²¹, and R²² are as defined for compound of formula (I) supra or in the examples below,
R¹⁴ is as defined for compound of formula (I) supra or in the examples below, or is an amine protecting group, such as a BOC group,
R¹⁵ is as defined for compound of formula (I) supra or in the examples below, or is an amine protecting group, such as a BOC group,
R¹⁸, is as defined for compound of formula (I) supra or in the examples below, or is an amine protecting group, such as a BOC group,
R¹⁹ is as defined for compound of formula (I) supra or in the examples below, or is an amine protecting group, such as a BOC group,
R²⁰ is as defined for compound of formula (I) supra or in the examples below, or is an amine protecting group, such as a BOC group,
wherein at least one of R¹⁴, R¹⁵, R¹⁸, R¹⁹, R²⁰ represents an amine protecting group, such as a BOC group.

Another aspect of the invention relates to the intermediates described herein and their use for preparing a compound of formula (I) as defined supra or an N-oxide, a salt, a tautomer or a stereoisomer of said compound, or a salt of said N-oxide, tautomer or stereoisomer.

The intermediates used for the synthesis of the compounds of claims 1 to 6 as described below, as well as their use for the synthesis of the compounds of claims 1 to 6, are one further aspect of the present invention. Preferred intermediates are the Intermediate Examples as disclosed below.

### EXPERIMENTAL SECTION

### General Part

Chemical names were generated using ACD/Name Batch Version 12.02. Stereodescriptors were manually adapted as defined above. In case there is discrepancy between the chemical name of a compound and its chemical structure, the chemical structure shall prevail. In some cases generally accepted names of commercially available reagents were used in place of ACD generated names.

The following table lists the abbreviations used in this paragraph and in the Intermediate Examples and Examples section as far as they are not explained within the text body. A comprehensive list of the abbreviations utilized by organic chemists of ordinary skill in the art appears presented in the first issue of each volume of the Journal of Organic Chemistry; this list is typically presented in a table entitled Standard List of Abbreviations. The abbreviations contained therein, and all abbreviations utilized by organic chemists of ordinary skill in the art are hereby incorporated by reference.

| Abbreviation | Meaning |
|---|---|
| BOC | tert-butoxycarbonyl- |
| br. | broad signal (NMR) |
| CI | chemical ionization |
| DABAL | (mu-1,4-diazabicyclo[2.2.2]octane-kappaN1: kappaN4) |
| | (hexamethyl)dialuminium [CAS No. 137203-34-0] |
| DAST | (diethylamino)sulfur trifluoride [CAS No. 38078-09-0] |
| DCM | dichloromethane |
| d | doublet (NMR), day(s) |
| dd | doublet of doublet (NMR) |
| dt | doublet of triplet (NMR) |
| DMF | N,N-dimethylformamide |
| DMSO | dimethylsulfoxide |
| EDTA | ethylenediaminetetraacetic acid |
| ESI | electrospray (ES) ionisation |
| GC-MS | gas chromatography-mass spectrometry |
| h | hour(s) |
| HATU | 1-[bis(dimethylamino)methylene]-1H-1,2,3-triazolo[4,5- |
| | b]-pyridinium 3-oxid hexafluorophosphate |
| HCl | hydrochloric acid |
| HPLC | high performance liquid chromatography |
| HRP | horseradish peroxidase |
| LCMS | liquid chromatography-mass spectrometry |
| m | multiplet (NMR) |
| min | minute(s) |
| MS | mass spectrometry |
| MTP | microtiter plate |
| NMR | nuclear magnetic resonance spectroscopy: chemical shifts |
| | (δ) are given in ppm. The chemical shifts were corrected |
| | by setting the DMSO signal to 2.50 ppm using unless |
| | otherwise stated. |
| NAD⁺ | nicotinamide adenine dinucleotide |
| PBS | phosphate buffered saline |
| PDC | pyridinium dichromate |
| PG | protecting group |
| Ph | phenyl |
| PyBOP | benzotriazol-1-yl-oxytripyrrolidinophosphonium |
| | hexafluorophosphate |
| q | quartet (NMR) |
| Rₜ | retention time (chromatography) |
| s | singulet (NMR) |
| SPA | Scintillation proximity assay |
| T3P | 1-propanephosphonic anhydride |
| TBAF | tetrabutylammonium fluoride |
| t | triplet (NMR) |
| td | triplet of doublet (NMR) |
| TFA | trifluoro acetic acid |
| THF | tetrahydrufuran |
| [³H]− | tritium |
| δ | chemical shift |

Other abbreviations have their meanings customary per se to the skilled person.

The various aspects of the invention described in this application are illustrated by the following examples which are not meant to limit the invention in any way.

### 1. Syntheses of Compounds (Overview)

The compounds of the present invention can be prepared as described in the following section. Schemes 1 to 17 and the procedures described below illustrate general synthetic routes to the compounds of general formula (I) of the invention and are not intended to be limiting. It is clear to the person skilled in the art that the order of transformations as exemplified in Schemes 1 to 17 can be modified in various ways. The order of transformations as exemplified in the Schemes 1 to 17 are therefore not intended to be limiting. In addition, interconversion of any of the substituents, R¹, R², R⁶, R⁷, R¹⁰, R¹¹ and —N(R¹⁰)R¹¹ can be achieved before and/or after the exemplified transformations. These modifications can be such as the introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs. Further, it is possible that two or more successive steps may be performed without work-up being performed between said steps, e.g. a “one-pot” reaction, as is well-known to the person skilled in the art.

In case mixtures of stereoisomers, such as, for example enantiomers, diastereomers, or cis/trans isomers are formed during a reaction, these isomers can be separated by methods described herein or by methods known to the person skilled in the art such as, but not limited to, chromatography, chiral chromatography and crystallization.

All reagents used for the preparation of the compounds of the invention are either commercially available or can be prepared as described.

### 1.1 Synthesis of Amines

Aliphatic amines as intermediates for the synthesis of compounds of the invention are either commercially available or can be synthesized as depicted in scheme 1 to 6 and scheme 15 to 17.

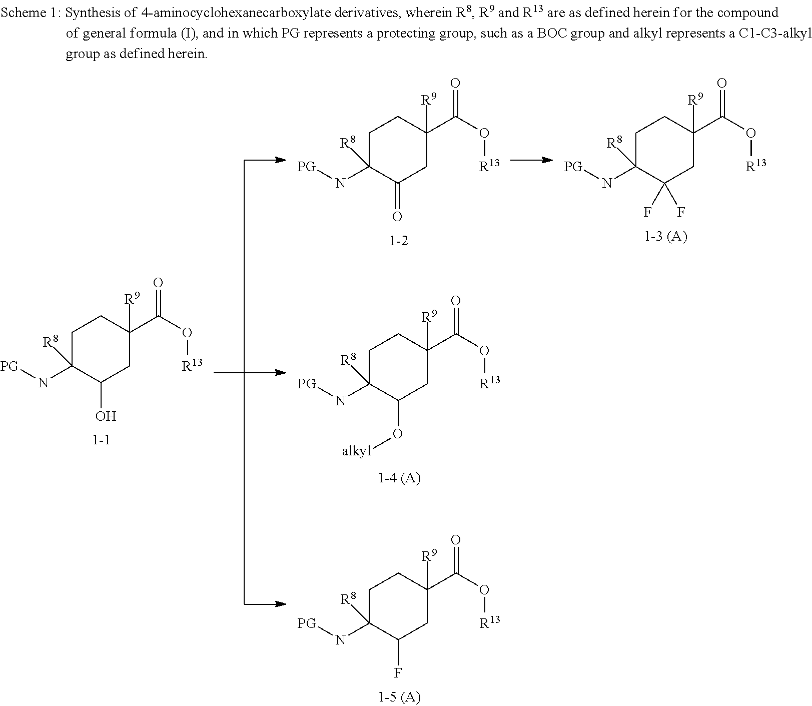

Treatment of 4-aminocyclohexanecarboxylates of type 1-1, which are commercially available or can be prepared according to literature procedures (for example see: I. Z. Siemion et al. Tetrahedron: Asymmetry 2001, 12, 455), where the amino function is bearing a protecting group such as, for example, a BOC group and the carboxylic acid is protected, for example as an ester group, with oxidizing reagents such as, for example pyridinium dichromate (PDC) yield compounds of type 1-2. Reaction of carbonyl derivatives of type 1-2 with fluorinating agents, such as for example (diethylamino)sulfur trifluoride (DAST), yield compounds of type 1-3.

4-Aminocyclohexanecarboxylates of type 1-1 can be treated with alkylating agents such as, for example alkyl halides, preferably alkyl iodides, bromides and chlorides, in the presence of, for example, silver (I) oxide to yield compounds of type 1-4.

Treatment of 4-aminocyclohexanecarboxylates of type 1-1, with fluorinating agents, such as for example 1,1,2,2,3,3,4,4,4-nonafluorobutane-1-sulfonyl fluoride, in the presence of a suitable base, such as for example, (1,8-diazabicyclo[5.4.0]undec-7-ene) (DBU) yield compounds of type 1-5.

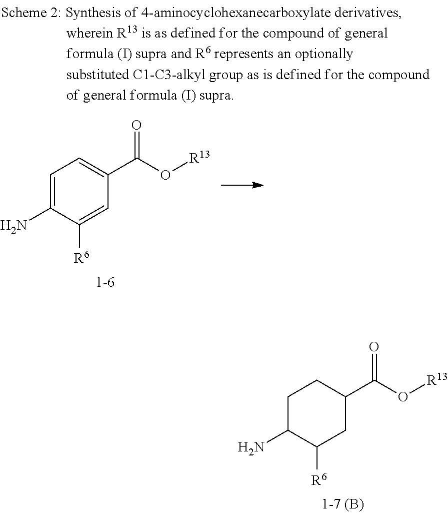

Hydrogenation of 4-aminobenzoic acid derivatives of type 1-6 under elevated pressure of hydrogen in the presence of a suitable catalyst, such as for example, rhodium on charcoal in a suitable solvent, such as for example ethanol, which might contain optionally acidic co-solvents, such as for instance acetic acid, gives cyclohexane derivatives of type 1-7 (B).

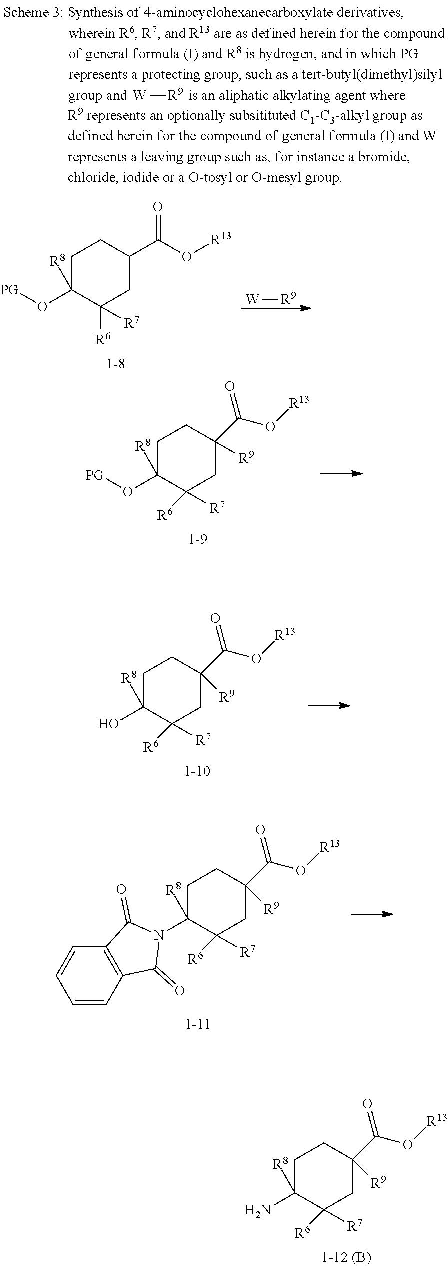

Cyclohexanecarboxylate derivatives of type 1-8, can be alkylated at the appropriate position by treatment with a suitable base such as for example, lithium diisopropylamide, in an appropriate solvent, such as for example tetrahydrofuran or diethylether, followed by addition of a suitable electrophile of type W—R⁹ to give compounds of type 1-9 (where R⁹ represents a optionally substituted C1-C3-alkyl group).

Alternatively, cyclohexanecarboxylate derivatives of type 1-8 react with a suitable base such as for example, lithium diisopropylamide, in an appropriate solvent, such as for example tetrahydrofuran or diethylether followed by addition of halogenating agents, such as for example N-fluorobenzenesulfonimide to give compounds of type 1-9 (where R⁹ represents a halogen, preferably a fluoride).

Deprotection of a protect alcohol of type 1-9, in case of a tert-butyl(dimethyl)silyl group for example, employing tetra-N-butylammonium fluoride (TBAF) results in alcohol derivatives of type 1-10.

Reaction of alcohol derivatives of type 1-10 with phthalimide under Mitsunobu reaction conditions employing for example diisopropyl azodicarboxylate (DIAD) and triphenylphosphine as reagents yields compounds of type 1-11.

Deprotection of the protected amine of type 1-11 bearing a phthalimide group can be achieved, for example, by treatment with hydrazine hydrate or methylamine at elevated temperature (e.g. at reflux) to give amine derivatives of type 1-12 (B).

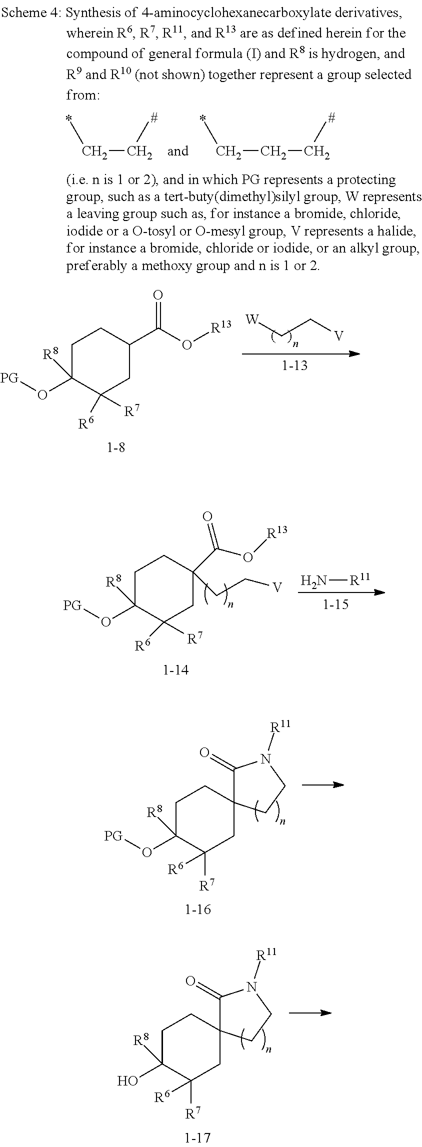

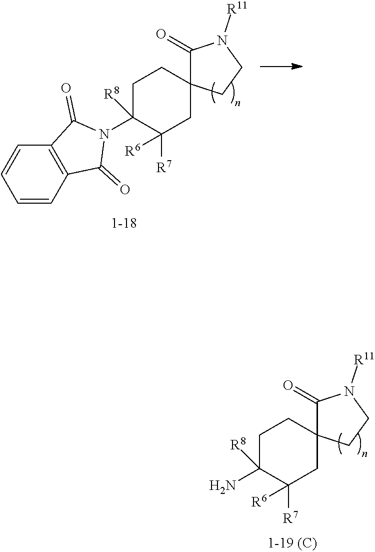

Cyclohexanecarboxylate derivatives of type 1-8, can be alkylated at the appropriate position by treatment with a suitable base such as, for example, lithium diisopropylamide, in an appropriate solvent such as, for example, tetrahydrofuran or diethylether, followed by addition of a suitable electrophile of type 1-13 to give compounds of type 1-14.

Reaction of compounds of type 1-14 (where V represents a halide, such as, for example, a chloride, bromide or idodide) with primary aromatic or heteroaromatic amines of type 1-15 in the presence of a base, such as, for instance, lithium bis(trimethylsilyl)amide, give compounds of type 1-16.

Alternatively compounds of type 1-16 can be obtained by reaction of compounds of type 1-14 (where V represents alkoxy group, such as for example, methoxy, and PG is preferably a hydrogen) with aromatic or heteroaromatic amines of type 1-15 in the presence of a lewis acid, such as for instance diethylaluminium chloride.

Compounds of type 1-19 can be obtained in three steps or 4 steps from compounds of type 1-16 in analogy the synthesis described in scheme 3 and scheme 15.

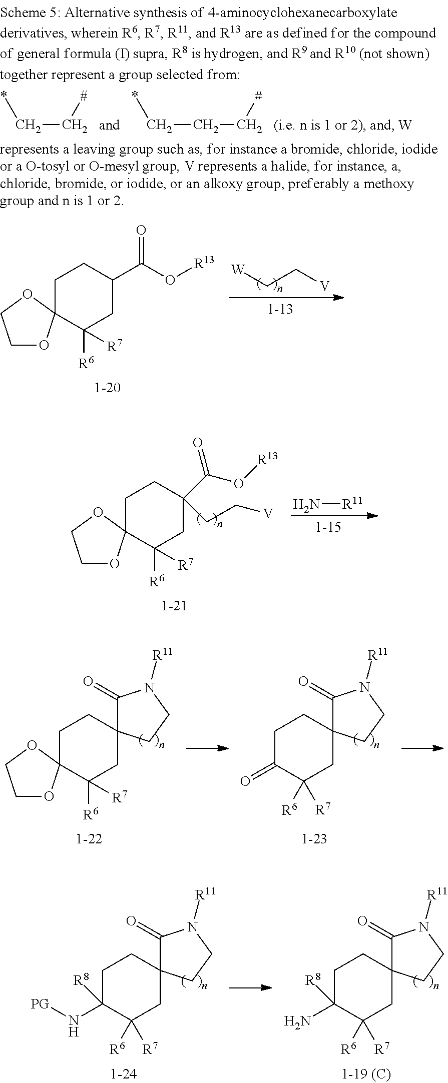

Cyclohexanecarboxylate derivatives of type 1-20, can be alkylated at the appropriate position by treatment with a suitable base such as for example, lithium hexamethyldisilazide, in an appropriate solvent such as for example tetrahydrofuran or diethylether, followed by addition of a suitable electrophile 1-13 to give compounds of type 1-21.

Reaction of compounds of type 1-21 (where V represents a halide) with primary amines of type 1-15 in the presence of a base, such as for instance lithium bis(trimethylsilyl)amide give compounds of type 1-22.

Treatment of compounds of type 1-22 with aqueous mineral acid give compounds of type 1-23.

Reaction of ketones of type 1-23 with amines, such as, for example, benzyl amine under standard reaction conditions for reductive animation reactions, employing for example sodium triacetoxyborhydride as reducing agent, yields protected amine derivatives of type 1-24.

Deprotection of the protected amine of type 1-24, in case of a benzyl protecting group for example, employing a palladium on charcoal catalyst and hydrogen gas, yields amine derivatives of type 1-19 (C).

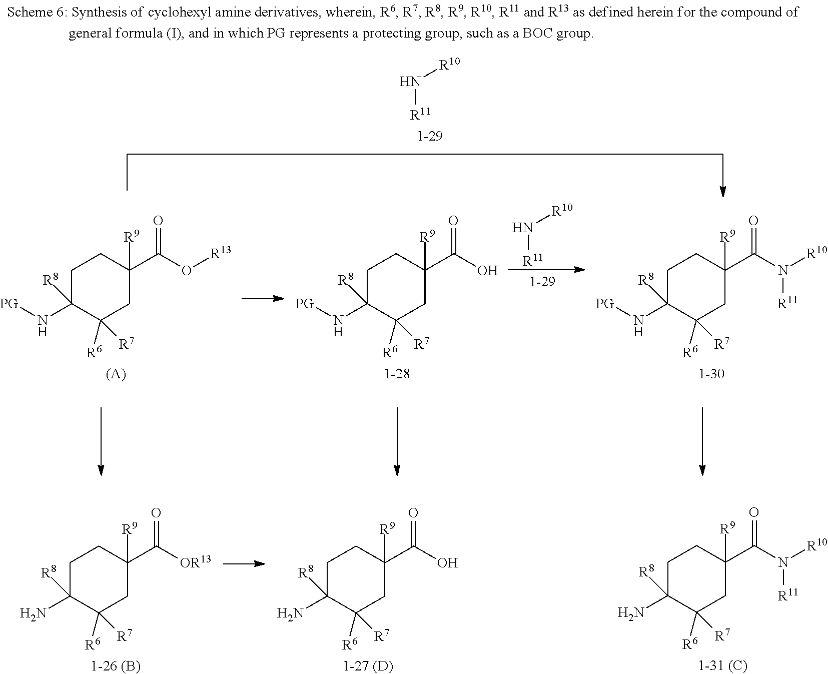

Compounds of type (A) may serve as starting materials for several transformations:

Reaction of a 4-aminocyclohexanecarboxylate derivative of type (A), where the amino function is bearing a protecting group such as, for example, a BOC group and the carboxylic acid is protected, for example as an ester group, preferably a methyl or ethyl ester, with an amine of the type 1-29 in presence of, for example, (mu-1,4-diazabicyclo[2.2.2]octane-kappaN1:kappaN4) (hexamethyl)dialuminium (DABAL) results in compounds auf type 1-30.

Alternatively compounds of type 1-30 can be obtained in a two-step procedure starting from compounds of general formula (A) by first, deprotection of the protected carboxylic acid, for example under basic conditions using for example lithium hydroxide to give compounds of type 1-28 followed by standard amide bond forming reaction with amines of type 1-29 in the presence of coupling agent such as, for example, HATU, T3P or the corresponding acid chloride intermediates of compounds of type 1-28 to give compounds of type 1-30.

Deprotection of the protected amine of type 1-30, in case of a BOC-protecting group, for example, employing trifluoroacetic acid or hydrochloric acid, results in compounds of type 1-31 (C).

Alternatively the protected amine of compounds of general formula 1-28 can be deprotected using, in the case of a BOC-protecting group, for example, trifluoroacetic acid or hydrochloric acid, to give compounds of general formula 1-27 (D).

Deprotection of the protected amine in compounds of the general formula (A) using, in the case of a BOC-protecting group for example, trifluoroacetic acid or hydrochloric acid, to give compounds of general formula 1-26 (B), which can be transformed to compounds of type 1-27 (D) by deprotection of the protected carboxylic acid, for example under basic conditions using, for example, lithium hydroxide to give compounds of general formula 1-27 (D).

### 1.2 Synthesis of Heteroaromatic Carboxylic Acid Derivatives

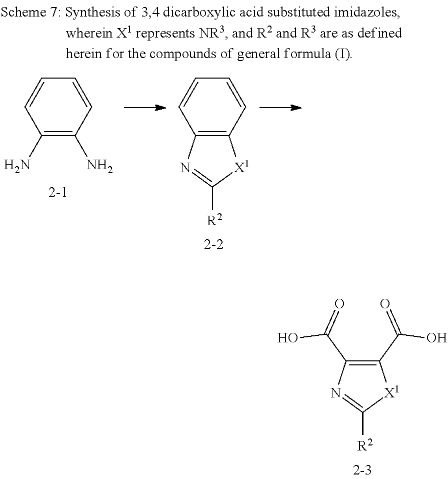

Commercially available benzene-1,2-diamine 2-1 can be reacted with carboxyclic acids at elevated temperatures (e.g. up to 200° C.) to give compounds of type 2-2.

Treatment of compounds of type 2-2, for example with hydrogenperoxide under acidic conditions and elevated temperature (e.g. at about 120° C.), yields compounds of type 2-3.

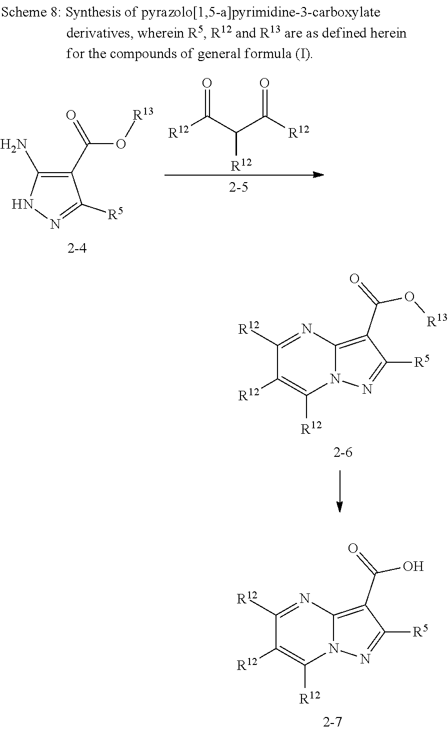

Commercially available, optionally substituted, 5-amino-1H-pyrazole-4-carboxylic acids of type 2-4 can be condensed with dicarbonyl compounds of type 2-5 at elevated temperatures (e.g. about 110° C.) in a suitable solvent such as for example acetic acid to give compounds of type 2-6.

Upon hydrolysis of the ester functionality of compounds of type 2-6 employing standard ester hydrolysis conditions, such as for example, an aqueous inorganic base, such as for example, lithium or sodium hydroxide, carboxylic acids of type 2-7 are obtained.

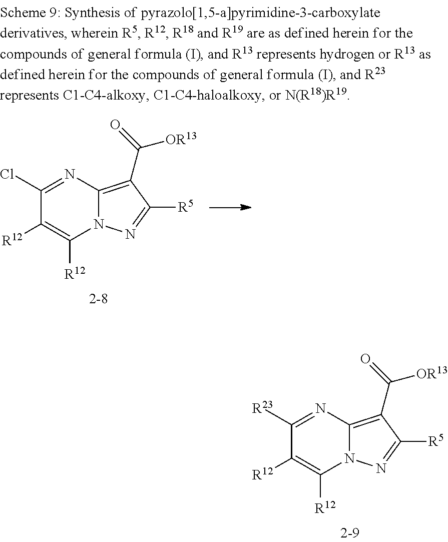

Reaction of compounds of type 2-8 with nucleophiles such as alcohols or primary or secondary amines in the presence of suitable base, for example a tertiary amine base, such as for example, N-ethyl-N-isopropylpropan-2-amine in the case of reaction with secondary amines, in a suitable solvent such as for example 2-propanol at elevated temperatures (e.g. at reflux) gives compounds of type 2-9.

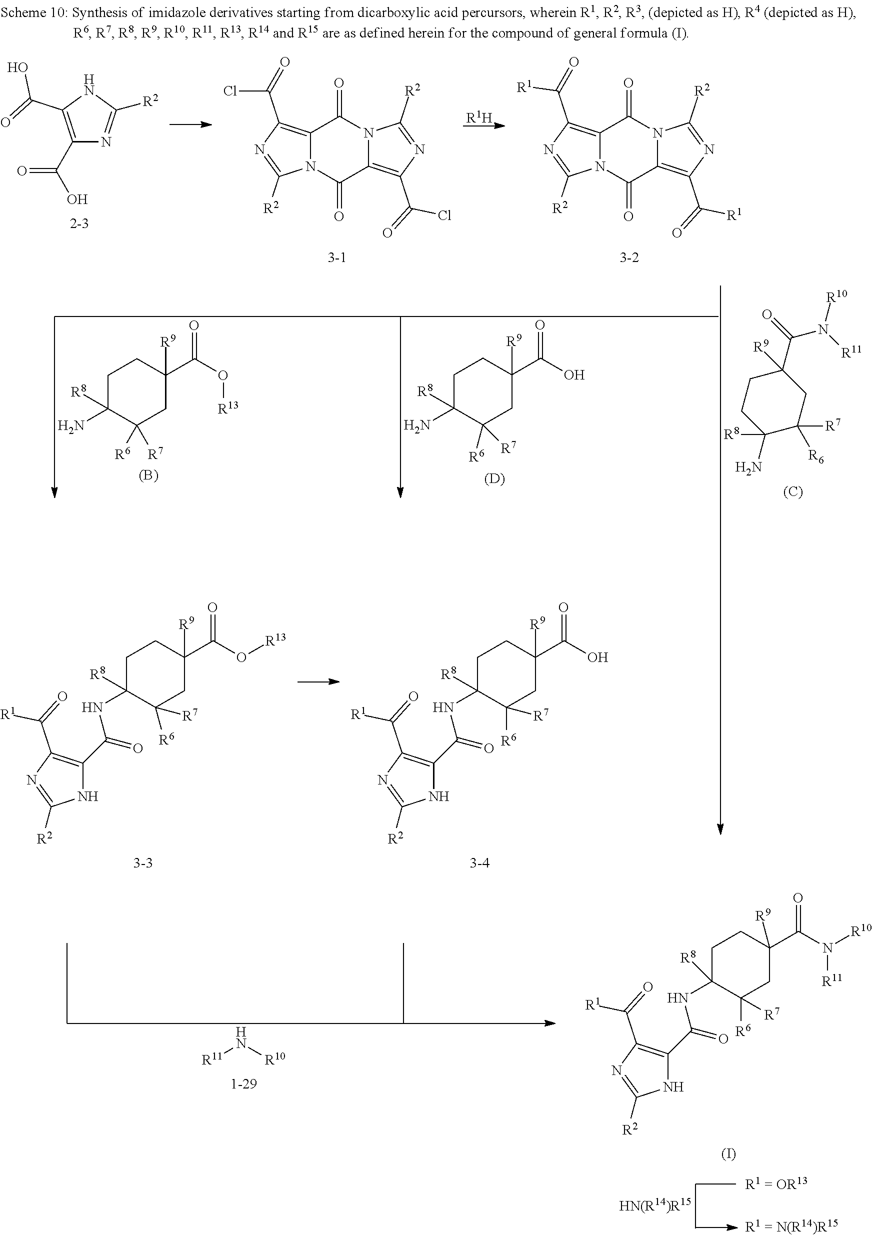

Starting from the corresponding 1H-imidazole-4,5-dicarboxylic acid derivatives of type 2-3, after treatment with thionylchloride at elevated temperature (e.g. at about 90° C.) 5,10-dioxo-5H,10H-diimidazo[1,5-a:1′,5′-d]pyrazine-1,6-dicarbonyl dichlorides of type 3-1 are obtained.

Compounds of type 3-1 can be reacted with suitable nucleophiles, such as, for example, amines or alcohols of general formula R¹H in presence of a suitable base, for example N-ethyl-N-isopropylpropan-2-amine, to give a compound of general formula 3-2.

Compounds of type 3-2 may serve as starting materials for several transformations:

Compounds of general formula (I) can be obtained directly by reacting compounds of general formula 3-2 with a fully decorated amine of general formula (C) at room temperature or elevated temperatures (e.g. at reflux).

Alternatively, an intermediate of type 3-4 can be obtained by reacting a compound of general formula 3-2 with suitably substituted amines of type (D) at elevated temperatures (e.g. at reflux) followed by standard amide bond forming reactions, for example with amines of the type 1-29 in presence of a coupling agent such as, for example, T3P or HATU or by reaction of an amine of the type 1-29 with the corresponding acid chloride of intermediates of type 3-4, to give compounds of formula (I).

Another alternative synthesis route employs compounds of general formula 3-2 in presence of amines of the type (B) with a suitably protected carboxylic acid function, such as, for example a methyl-protecting group, at room temperature or elevated temperatures to give compounds of type 3-3.

Deprotection of the protected carboxylic acid, for example under basic conditions using lithium hydroxide results in compounds of type 3-4, which can then be transformed further as described above. Alternatively, compounds of type 3-3 can be directly transformed to compounds of formula (I) by reaction with an suitable amine of type 1-29 in the presence of for example DABAL.

Esters of formula (I) (i.e. compounds of formula (I) wherein R¹ represents OR¹³) can be transformed into amides of general formula (I) (i.e. compounds of formula (I) wherein R¹ represents —N(R¹⁴)R¹⁵), according to the invention, for example by treatment with different amines of formula HN(R¹⁴)R¹⁵, optionally in presence of a base, such as, for example, N-ethyl-N-isopropylpropan-2-amine, or in the presence of reagents such as DABAL, or alternatively in a two-step procedure consisting of ester hydrolysis, for example using sodium hydroxide followed by standard amide bond formation in presence of amines and coupling agents such as HATU or alternatively in a three step procedure after hydrolysis of the ester, generation of corresponding acid chloride, for example using thionylchloride and reaction with amines under basic conditions in presence of, for example, N-ethyl-N-isopropylpropan-2-amine.

An alternative synthesis route of derivatives of the present invention is depicted in scheme 11.

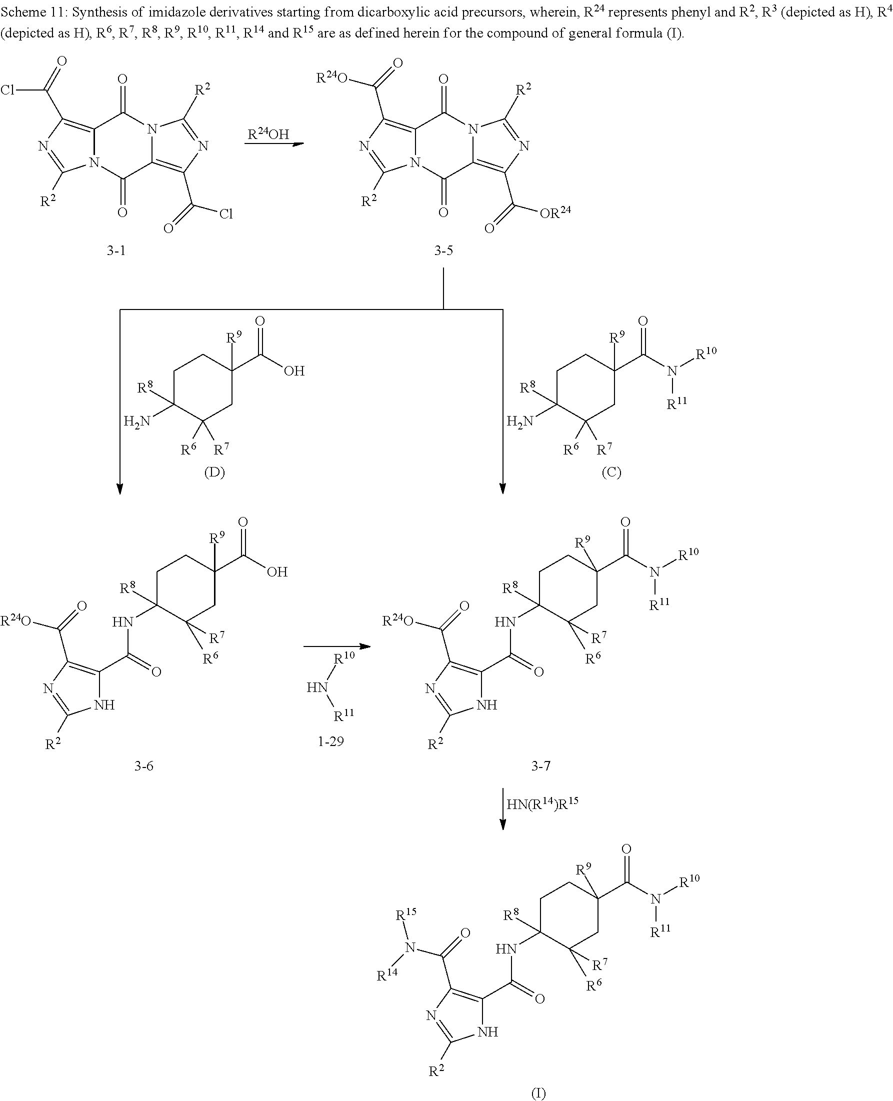

Compounds of type 3-1 (preparation see above) can be reacted with phenol in presence of a suitable base, for example pyridine, to give a compound of general formula 3-5.

Compounds of type 3-5 may serve as starting materials for several transformations: Compounds of general formula 3-7 can be obtained directly by reacting compounds of general formula 3-5 with a fully decorated amine of general formula (C) at room temperature or elevated temperatures (e.g. at reflux).

Alternatively, an intermediate of type 3-6 can be obtained by reacting a compound of general formula 3-5 with suitably substituted amines of type (D) at elevated temperatures (e.g. at reflux) followed by standard amide bond forming reaction with amines of the type 1-29 in presence of a coupling agent such as, for example, T3P, HATU or PyBOP or by reaction of an amine of the type 1-29 with the corresponding acid chloride of intermediates of type 3-6, to give compounds of type 3-7.

Phenyl esters of type 3-7 can be transformed into amides of general formula (I) (i.e. compounds of formula (I) wherein R¹ represents —N(R¹⁴)R¹⁵), according to the invention, for example by treatment with different amines of formula HN(R¹⁴)R¹⁵, optionally in presence of a base, such as, for example, N-ethyl-N-isopropylpropan-2-amine, or in the presence of reagents such as DABAL, or alternatively in a two-step procedure consisting of ester hydrolysis, for example using sodium hydroxide followed by standard amide bond formation in presence of amines and coupling agents such as HATU or alternatively in a three step procedure after hydrolysis of the ester, generation of corresponding acid chloride, for example using thionylchloride and reaction with amines under basic conditions in presence of, for example, N-ethyl-N-isopropylpropan-2-amine.

An alternative synthesis route of heterocylic derivatives of the present invention is depicted in scheme 12.

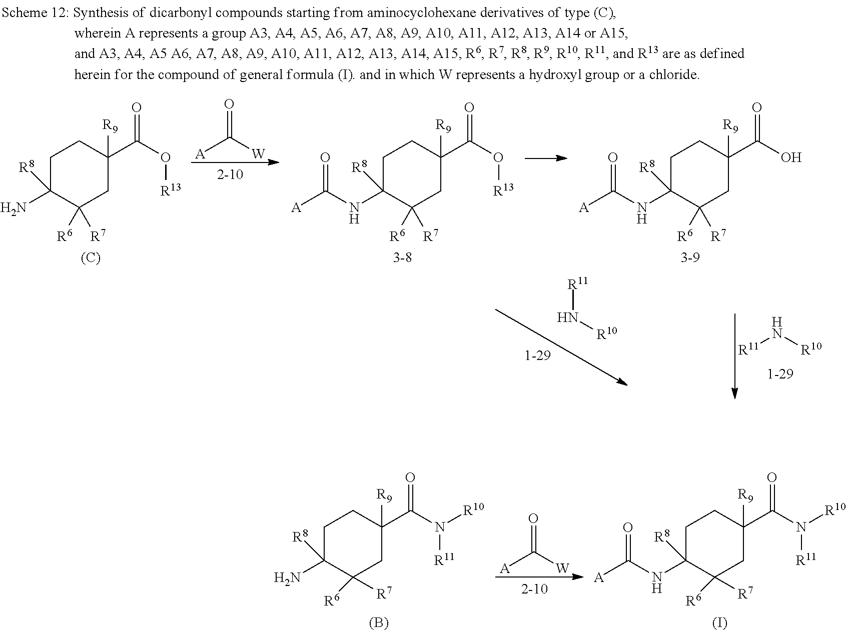

Starting from amino cyclohexane derivatives of type (C), where the carboxylic acid is protected, for example as an ester group, upon standard amide bond forming reaction condition, for example, using a carboxylic acid of type 2-10 in the presence of a coupling agent such as, for example, PyBOP or the corresponding acid chloride, compounds of type 3-8 are obtained. Upon hydrolysis of the ester functionality of compounds of type 3-8 employing standard ester hydrolysis conditions, such as, for example, an aqueous inorganic base, such as, for example, lithium or sodium hydroxide, carboxylic acids of type 3-9 are obtained. Reaction of carboxylic acids of type 3-9 with amines of type 1-29 under standard amide bond forming reaction conditions, for example using a coupling agent such as, for example, PyBOP give compounds of formula (I). Alternatively, carboxylic acids of type 3-9 can be converted to the corresponding acid chlorides applying chlorinating agents, such as, for example, thionyl chloride or 1-chloro-N,N,2-trimethylprop-1-en-1-amine, followed by reaction with amines of type 1-29 to give compounds of formula (I).

Alternatively compounds of type 3-8 can be converted directly to compounds of formula (I) by reaction with an amine of type 1-29 in the presence of, for example, DABAL.

Alternatively, compounds of formula (I) can be obtained starting from amino cyclohexane derivatives of type (B), upon standard amide bond forming reaction condition, for example using a carboxylic acid of type 2-10 in the presence of a coupling agent such as, for example, PyBOP or the corresponding acid chloride of compounds of type 2-10.

### 1.3 Synthesis of Oxazole Derivatives

Yet another possible synthesis route for the compounds of this invention is depicted in schemes 13 and 14.

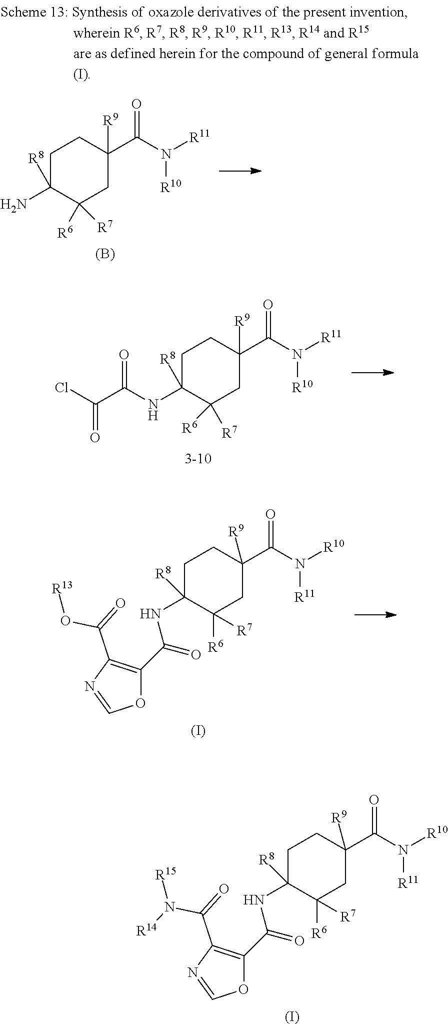

Compounds of type (B) can be transformed into compounds of type 3-10 by reaction with oxalyl chloride.

Reaction of compounds of type 3-10 with alkyl isocyanoacetates in presence of, for example, imidazole and triethylamine yields esters of general formula (I) (i.e. compounds of formula (I) wherein R¹ represents OR¹³) as claimed in this invention.

Esters of general formula (I) can be transformed into amides of general formula (I) (i.e. compounds of formula (I) wherein R¹ represents —N(R¹⁴)R¹⁵), according to the invention, for example by treatment with different amines of formula HN(R¹⁴)R¹⁵, optionally in presence of a base, such as, for example, N-ethyl-N-isopropylpropan-2-amine, or alternatively in a two step procedure consisting of ester hydrolysis, for example using sodium hydroxide followed by standard amide bond formation in presence of amines and coupling agents such as HATU or alternatively in a three step procedure after hydrolysis of the ester, generation of corresponding acid chloride, for example using thionylchloride and reaction with amines under basic conditions in presence of, for example, N-ethyl-N-isopropylpropan-2-amine.

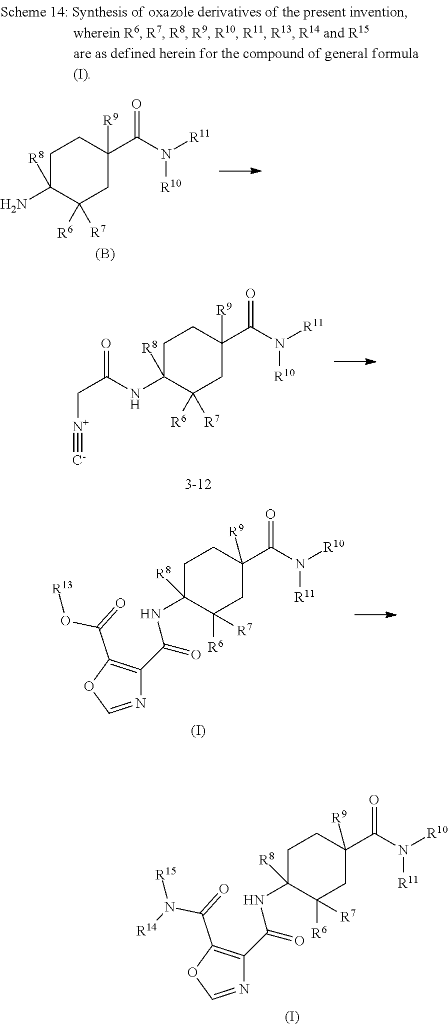

Compounds of type (B) can be transformed into compounds of type 3-12 by reaction with potassium isocyanoacetate in the presence of a suitable base such as for example N-ethyl-N-isopropylpropan-2-amine and HATU.

Reaction of general formula 3-12 with methyl chloro(oxo)acetate in presence of, for example, imidazole and triethylamine yields esters of general formula (I) (i.e. compounds of formula (I) wherein R¹ represents OR¹³) as claimed in this invention.

Esters of general formula (I) can be transformed into amides of general formula (I) (i.e. compounds of formula (I) wherein R¹ represents —N(R¹⁴)R¹⁵), according to the invention, for example by treatment with different amines of formula HN(R¹⁴)R¹⁵, optionally in presence of a base, such as, for example, N-ethyl-N-isopropylpropan-2-amine, or alternatively in a two-step procedure consisting of ester hydrolysis, for example using sodium hydroxide followed by standard amide bond formation in presence of amines of formula HN(R¹⁴)R¹⁵ and coupling agents such as HATU or alternatively in a three step procedure after hydrolysis of the ester, generation of corresponding acid chloride, for example using thionylchloride and reaction with amines of formula HN(R¹⁴)R¹⁵ under basic conditions in presence of, for example, N-ethyl-N-isopropylpropan-2-amine.

### 1.4 Alternative Synthesis of Amines

An alternative synthesis route of derivatives of the present invention is depicted in scheme 15.

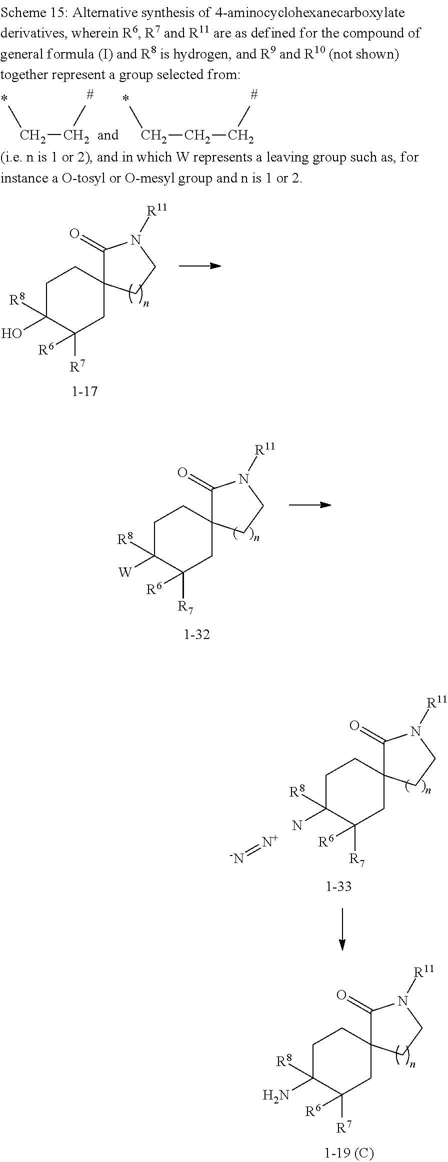

Reaction of the alcohol derivatives of type 1-17 with for example W-Cl in the presence of a suitable base for example pyridine yields compounds of type 1-32. Treatment of compounds of type 1-32 with sodium azide in a suitable solvent, such as for instance DMF yield compounds of type 1-33. Reduction of acid derivatives of type 1-33 with for instance triphenylphosphane give compounds of type 1-19.

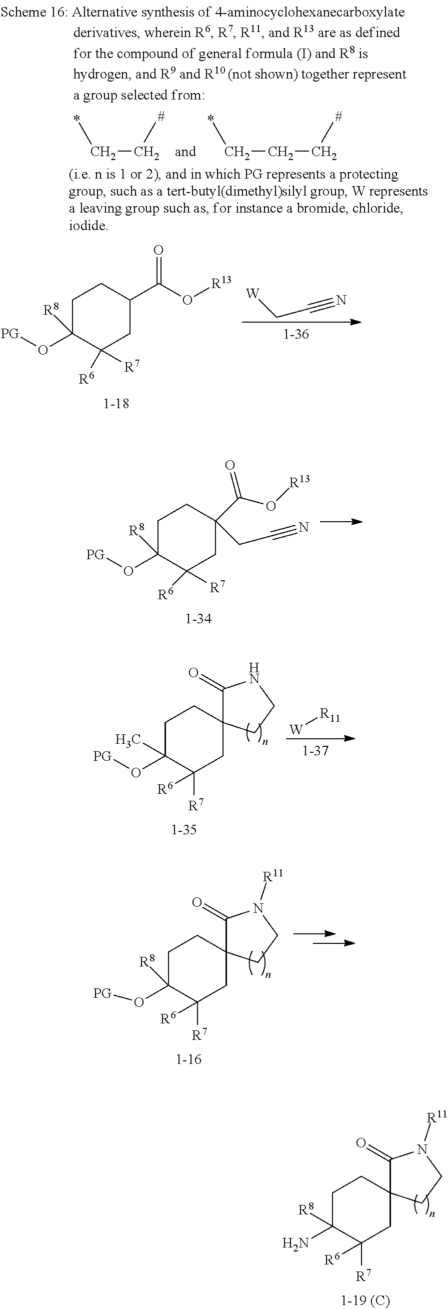

Cyclohexanecarboxylate derivatives of type 1-8, can be alkylated at the appropriate position by treatment with a suitable base such as, for example, lithium diisopropylamide, in an appropriate solvent such as, for example, tetrahydrofuran or diethylether, followed by addition of a suitable electrophile of type 1-36 to give compounds of type 1-34.

Compounds of type 1-34 can be reduced and cyclized to compounds of type 1-35 using for instance, raney nickel in ammonia and under an hydrogen atmosphere.

Compounds of type 1-16 can be obtained by reaction of compounds of type 1-35 with compounds of type 1-37 in the presence of a catalyst, such for example copper(I)iodide and bases such as N,N′-dimethylethylenediamine and potassium carbonate in a suitable solvent like for example dixane.

Compounds of type 1-19 can be obtained in three steps from compounds of type 1-16 in analogy the synthesis described in scheme 4 or scheme 15.

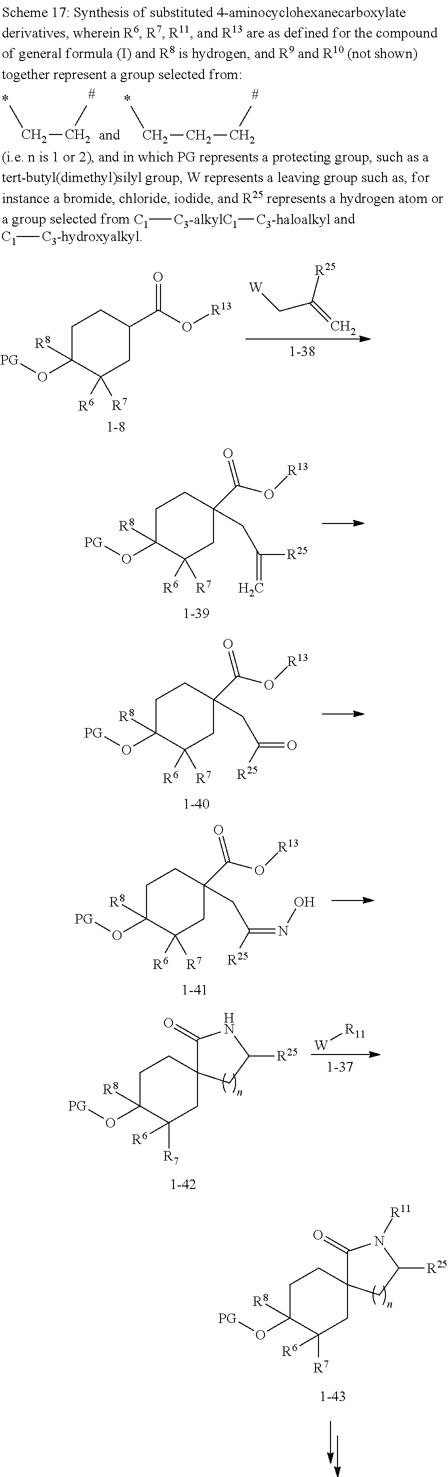

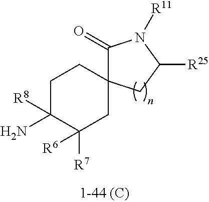

Cyclohexanecarboxylate derivatives of type 1-8, can be alkylated at the appropriate position by treatment with a suitable base such as, for example, lithium diisopropylamide, in an appropriate solvent such as, for example, tetrahydrofuran or diethylether, followed by addition of a suitable electrophile of type 1-38 to give compounds of type 1-39.

Ozonolysis of compounds of type 1-39 yields compounds of type 1-40. Compounds of type 1-40 can be reacted with hydroxyl amine in the presence of a suitable base such as triethyl amine to give compounds of type 1-41.

Compounds of type 1-41 can be reduced and cyclized to compounds of type 1-42 using for instance, Pd/C in methanol and under an hydrogen atmosphere followed by treatment with an suitable base such as triethylamin in a suitable solvent such as toluene at elevated temperatures.

Compounds of type 1-43 can be obtained by reaction of compounds of type 1-42 with compounds of type 1-37 in the presence of a catalyst, such for example copper(I)iodide and bases such as N,N′-dimethylethylenediamine and potassium carbonate in a suitable solvent like for example dixane.

Compounds of type 1-44 (C) can be obtained in three or four steps from compounds of type 1-43 in analogy the synthesis described in scheme 4 or scheme 15.

### NMR Data:

NMR peak forms are stated as they appear in the spectra, possible higher order effects have not been considered.

The ¹H-NMR data of selected examples are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensity), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight”<1%.

### Analytical HPLC Methods:

### Method 1:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method 2:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Method 3:

Instrument: Waters Acquity UPLCMS SingleQuad; Column: Acquity UPLC BEH C18 1.7 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Method 4:

Instrument: Waters Acquity UPLCMS SingleQuad; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method 5:

Instrument: Waters Acquity UPLCMS SingleQuad; column: BEH C 18 (Waters) 1.7 μm, 50×2.1 mm; eluent A: water+0.05 Vol-% formic acid (99%), eluent B: acetonitrile+0.05% formic acid; gradient: 0-0.2 min 98% A, 0.2-1.7 min 98-10% A, 1.7-1.9 min 10% A, 1.9-2.0 min 10-98% A, 2.0-2.5 min 98% A; flow 1.3 ml/min; temperature: 60° C.; DAD scan: 210-400 nm

### Method 6:

Instrument: Agilent 1290 UHPLCMS Tof; column: BEH C 18 (Waters) 1.7 μm, 50×2.1 mm; eluent A: water+0.05 Vol-% formic acid (99%), eluent B: acetonitrile+0.05% formic acid; gradient: 0-1.7 min 98-10% A, 1.7-2.0 min 10% A, 2.0-2.5 min 10-98% A, flow 1.2 ml/min; temperature: 60° C.; DAD scan: 210-400 nm

### Method 7:

Instrument: Waters Acquity UPLCMS SingleQuad; column: Kinetex C 18 (Phenomenex) 2.6 μm, 50×2.1 mm; eluent A: water+0.05 Vol-% formic acid (99%), eluent B: acetonitrile+0.05% formic acid; gradient: 0-0.2 min 98% A, 0.2-1.7 min 98-10% A, 1.7-1.9 min 10% A, 1.9-2.0 min 10-98% A, 2.0-2.5 min 98% A; flow 1.3 ml/min; temperature: 60° C.; DAD scan: 210-400 nm

### Preparative HPLC Methods:

### Method 8:

Instrument: Waters Autopurification MS SingleQuad; Column: Waters XBrigde C18 5p 100×30 mm; eluent A: water+0.1 vol % formic acid (99%), eluent B: acetonitrile; gradient eluent A/eluent B, flow 70 ml/min; temperature: 25° C.; DAD scan: 210-400 nm.

### Method 9:

Instrument: Waters Autopurification MS SingleQuad; Column: Waters XBrigde C18 5μ 100×30 mm; eluent A: water+0.2 vol % aqueous ammonia (32%), eluent B: acetonitrile; gradient: eluent A/eluent B; flow 70 ml/min; temperature: 25° C.; DAD scan: 210-400 nm.

### Analytical GC-MS Methods:

### Method 10

Instruments: Agilent 7890A and Waters GCT Premier; Column: 29 m HP-5MS, 0.25 mm/0.25 μm; Gas: Helium 1 ml/min, Oven: Start 50° C. 1 min, linear to 260° C. at 10° C./min.

### Specific Optical Rotation Methods:

### Method 11

Instrument: JASCO P2000 Polarimeter; wavelength 589 nm; temperature: 20° C.; integration time 10 s; path length 100 mm.

### 3. Intermediates

### Intermediate I1

### 5,10-dioxo-5H,10H-diimidazo[1,5-a:1′,5′-d]pyrazine-1,6-dicarbonyl dichloride

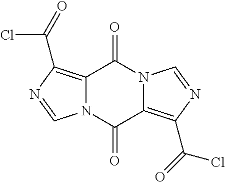 [SMILES: O=C(Cl)c1ncn2c(=O)c3c(C(=O)Cl)ncn3c(=O)c12]

In a dried flask, to 25 g (157 mmol) 1H-imidazole-4,5-dicarboxylic acid in 334 ml of toluene, 12.1 ml DMF and 94 ml (1.29 mol) thionyl chloride were added. The mixture was stirred for 24 h at 80° C. The mixture was concentrated under reduced pressure. 100 ml toluene were added and the mixture was concentrated under reduced pressure to give 35.5 g of the title compound as crude material which was used at the same day without further purification for subsequent steps.

### Intermediate I2

### Diphenyl 5,10-dioxo-5H,10H-diimidazo[1,5-a:1′,5′-d]pyrazine-1,6-dicarboxylate

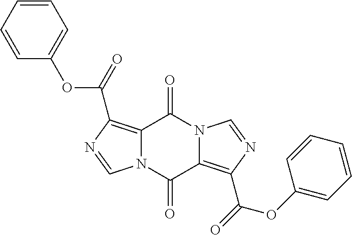 [SMILES: O=C(Oc1ccccc1)c1ncn2c(=O)c3c(C(=O)Oc4ccccc4)ncn3c(=O)c12]

20.0 g (crude product) of 5,10-dioxo-5H,10H-diimidazo[1,5-a:1′,5′-d]pyrazine-1,6-dicarbonyl dichloride and 12.6 g (0.13 mol) phenol were suspended in 380 ml dichloromethane. The resulting mixture was cooled to 0° C. and 10.8 ml (0.13 mol) pyridine were added dropwise. The reaction was stirred for 3 hours at room temperature. The precipitate was filtered off and washed with dichloromethane. The obtained solid material was dried under vacuum at 50° C. to give 21.6 g of the title compound as a crude product which was used without further purification in the subsequent steps.

### Intermediate I3

### trans-4-({[4-(phenoxycarbonyl)-1H-imidazol-5-yl]carbonyl}amino)cyclohexane-carboxylic acid

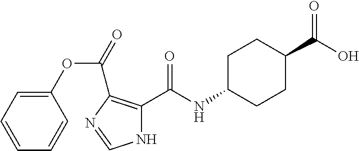 [SMILES: O=C(Oc1ccccc1)c1nc[nH]c1C(=O)N[C@H]1CC[C@H](C(=O)O)CC1]

To a suspension of 7.58 g (97%, 51.4 mmol) trans-4-aminocyclohexanecarboxylic acid and 11 g (25.7 mmol) diphenyl 5,10-dioxo-5H,10H-diimidazo[1,5-a:1′,5′-d]pyrazine-1,6-dicarboxylate in 800 ml tetrahydrofuran were added 7.16 ml (51.4 mmol) triethylamine. After stirring for 7 h at 90° C. 2.00 g (97% 13.9 mmol) trans-4-aminocyclohexanecarboxylic acid were added and the mixture was stirred for 24 h at 90° C. The precipitate was filtered off and the filtrate was concentrated in vacuo to 200 ml. The remaining organic phase was poured into 1 l dest. water and the mixture was acidified to a pH of 3. The resulting precipitate was filtered off and washed with water until a neutral pH was reached. The obtained solid material was dried under vacuum at 60° C. to give 14.7 g of the title compound as a crude product which was used without further purification in the subsequent steps.

LCMS (Method 1): Rₜ=0.84 min; MS (ESIpos) m/z=407.3 [M+H]⁺.

### Intermediate I4

### phenyl 5-({trans-4-[(3-chloropyridin-4-yl)carbamoyl]cyclohexyl}carbamoyl)-1H-imidazole-4-carboxylate

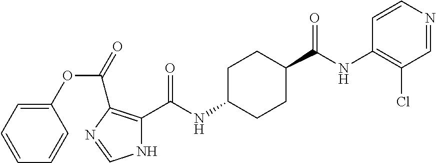 [SMILES: O=C(Oc1ccccc1)c1nc[nH]c1C(=O)N[C@H]1CC[C@H](C(=O)Nc2ccncc2Cl)CC1]

To a suspension of 250 mg (0.70 mmol) trans-4-({[4-(phenoxycarbonyl)-1H-imidazol-5-yl]carbonyl}amino)cyclohexane-carboxylic acid in 20 ml dichloromethane 195 μl (1.40 mmol) 1-chloro-N,N,2-trimethylprop-1-en-1-amine were added and the mixture was stirred at room temperature for 1 hour. 170 μl (2.10 mmol) pyridine and 92.7 mg (0.70 mmol) 3-chloropyridin-4-amine were added and the reaction was stirred at room temperature for 24 hours. The reaction mixture was concentrated in vacuo to obtain the crude product. Another batch was prepared accordingly (1.4 mmol of the starting material) and the combined crude products were purified by flash column chromatography (dichloromethane/methanol-gradient) to give 390 mg of the title compound as a solid material.

### Intermediate I5

### phenyl 5-({trans-4-[(2-chloro-4-fluorophenyl)carbamoyl]cyclohexyl}carbamoyl)-1H-imidazole-4-carboxylate

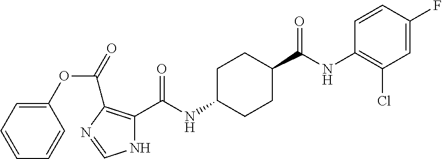 [SMILES: O=C(Oc1ccccc1)c1nc[nH]c1C(=O)N[C@H]1CC[C@H](C(=O)Nc2ccc(F)cc2Cl)CC1]

To a suspension of 3.00 g (8.40 mmol) trans-4-({[4-(phenoxycarbonyl)-1H-imidazol-5-yl]carbonyl}amino)cyclohexane-carboxylic acid in 140 ml dichloromethane 2.31 ml (16.8 mmol) 1-chloro-N,N,2-trimethylprop-1-en-1-amine were added and the mixture was stirred at room temperature for 1 hour. 2.04 ml (25.2 mmol) pyridine and 1.11 ml (9.23 mmol) 2-chloro-4-fluoroaniline were added and the reaction was stirred at room temperature for 3 days. The reaction mixture was concentrated in vacuo and the residue triturated with methanol to obtain 2.34 g of the title compound as a solid material.

LCMS (Method 2): Rₜ=1.01 min; MS (ESIpos) m/z=485.0 [M+H]⁺.

### Intermediate I6

### tert-butyl {trans-4-[(2-chloro-4-fluorophenyl)carbamoyl]cyclohexyl}carbamate

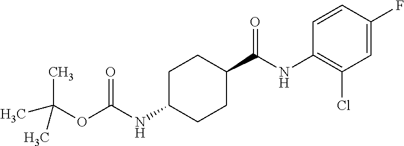 [SMILES: CC(C)(C)OC(=O)N[C@H]1CC[C@H](C(=O)Nc2ccc(F)cc2Cl)CC1]

1-Chloro-N,N,2-trimethylprop-1-en-1-amine (8.7 ml, 66 mmol) was added to a suspension of trans-4-[(tert-butoxycarbonyl)amino]cyclohexanecarboxylic acid (10.0 g, 41.1 mmol) in dichloromethane (500 ml) and the mixture was stirred for 30 min at room temperature. Pyridine (13 ml, 160 mmol) and 2-chloro-4-fluoroaniline (7.4 ml, 62 mmol) were added and the mixture was stirred over night at room temperature. For work-up, water was added and the mixture was extracted with dichloromethane followed by extraction with a mixture of dichloromethane/2-propanol. The combined organic phases were washed with saturated sodium bicarbonate solution and water, filtrated through a silicone filter and concentrated. The residue was stirred with diethyl ether and precipitate was collected by filtration and dried to give the title compound (11.2 g).

¹H-NMR (400 MHz, DMSO-d₆): δ [ppm]=9.45 (s, 1H), 7.59 (dd, 1H), 7.48 (dd, 1H), 7.20 (td, 1H), 6.76 (d, 1H), 3.26-3.04 (m, 1H), 2.43-2.18 (m, 1H), 1.91-1.70 (m, 4H), 1.52-1.29 (m, 11H), 1.25-1.07 (m, 2H)

### Intermediate I7

### trans-4-amino-N-(2-chloro-4-fluorophenyl)cyclohexanecarboxamide hydrochloric acid salt

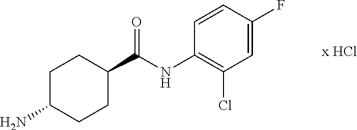 [SMILES: N[C@H]1CC[C@H](C(=O)Nc2ccc(F)cc2Cl)CC1]

Hydrochloric acid (113 ml, 4 M solution in dioxane, 450 mmol) was added to a suspension of tert-butyl {trans-4-[(2-chloro-4-fluorophenyl)carbamoyl]cyclohexyl}carbamate (11.2 g, 30.2 mmol) in dichloromethane (600 ml) and the mixture was stirred over night at room temperature. The precipitate was collected by filtration, washed with dichloromethane and dried to give the title compound (9.27 g).

LC-MS (Method 2): Rₜ=0.89 min; MS (ESIPos): m/z=271.1 [M+H]⁺

¹H-NMR (400 MHz, DMSO-d₆): δ [ppm]=9.54 (s, 1H), 7.94 (br. s., 3H), 7.59 (dd, 1H), 7.49 (dd, 1H), 7.21 (td, 1H), 3.06-2.91 (m, 1H), 2.46-2.34 (m, 1H), 2.07-1.79 (m, 4H), 1.57-1.28 (m, 4H)

### Intermediate I8

### tert-butyl {trans-4-[(4-chloropyridin-3-yl)carbamoyl]cyclohexyl}carbamate

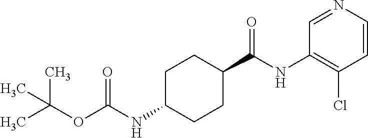 [SMILES: CC(C)(C)OC(=O)N[C@H]1CC[C@H](C(=O)Nc2cnccc2Cl)CC1]

1-Chloro-N,N,2-trimethylprop-1-en-1-amine (4.0 ml, 33 mmol) is added to a suspension of trans-4-[(tert-butoxycarbonyl)amino]cyclohexanecarboxylic acid (5.05 g, 20.7 mmol) in dichloromethane (350 ml) and the mixture was stirred for 30 min. Pyridine (10 ml, 120 mmol) and 4-chloropyridin-3-amine (3.7 ml, 31 mmol) were added and the mixture was stirred for 3 days at room temperature. For work-up, water was added and the mixer was extracted with dichloromethane. The combined organic phases were washed with saturated sodium bicarbonate solution and water, filtrated through a silicone filter and concentrated. The residue was stirred with methanol, the precipitate was collected by filtration and dried to give the title compound (6.88 g, 94% yield)

LC-MS (Method 1): Rₜ=0.99 min; MS (ESIpos): m/z=354 [M+H]⁺

¹H-NMR (400 MHz, DMSO-d₆): δ [ppm]=9.69 (s, 1H), 8.72 (s, 1H), 8.33 (d, 1H), 7.60 (d, 1H), 6.76 (d, 1H), 3.28-3.10 (m, 1H), 2.44-2.35 (m, 1H), 1.93-1.71 (m, 4H), 1.56-1.33 (m, 11H), 1.28-1.11 (m, 2H)

### Intermediate I9

### trans-4-amino-N-(4-chloropyridin-3-yl)cyclohexanecarboxamide hydrochloric acid salt

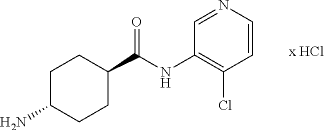 [SMILES: N[C@H]1CC[C@H](C(=O)Nc2cnccc2Cl)CC1]

Hydrochloric acid (73 ml, 4 M in dioxane, 290 mmol) was added to a suspension of tert-butyl {trans-4-[(4-chloropyridin-3-yl)carbamoyl]cyclohexyl}carbamate (6.88 g, 19.4 mmol) in dichloromethane (210 ml) and the mixture was stirred for 3 days. The precipitate was collected by filtration and dried to give the title compound (1.20 g, 21% yield).

LC-MS (Method 2): Rₜ=0.65 min; MS (ESIneg): m/z=252 [M−H]⁻

1H-NMR (400 MHz, DMSO-d6) delta [ppm]: 1.327 (0.79), 1.334 (0.85), 1.335 (0.88), 1.358 (2.37), 1.363 (2.65), 1.369 (2.23), 1.391 (3.48), 1.392 (3.48), 1.398 (3.25), 1.420 (2.37), 1.434 (2.58), 1.436 (2.42), 1.461 (3.66), 1.465 (3.74), 1.469 (3.30), 1.491 (2.88), 1.498 (3.27), 1.500 (3.25), 1.524 (1.09), 1.527 (1.04), 1.533 (0.99), 1.883 (0.76), 1.900 (3.30), 1.906 (3.90), 1.909 (3.97), 1.932 (2.88), 1.939 (3.20), 1.944 (3.06), 1.978 (3.44), 1.981 (3.62), 1.988 (3.43), 2.010 (3.55), 2.015 (3.27), 2.020 (2.98), 2.300 (0.76), 2.304 (1.08), 2.309 (0.78), 2.451 (1.47), 2.519 (1.64), 2.527 (0.88), 2.642 (0.81), 2.646 (1.09), 2.651 (0.78), 2.950 (0.69), 2.964 (1.04), 2.977 (1.40), 2.991 (1.41), 3.005 (1.02), 3.019 (0.64), 3.540 (0.85), 7.710 (9.68), 7.724 (10.21), 7.743 (0.69), 8.041 (5.81), 8.054 (5.81), 8.377 (12.03), 8.391 (11.60), 8.403 (0.95), 8.811 (16.00), 8.830 (1.06), 9.912 (8.14).

### Intermediate I10

### trans-4-amino-N-(3-chloropyridin-4-yl)cyclohexanecarboxamide hydrochloric acid salt

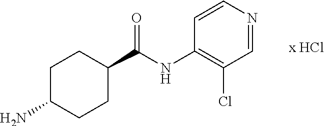 [SMILES: N[C@H]1CC[C@H](C(=O)Nc2ccncc2Cl)CC1]

Was prepared in 2 steps in analogy to the synthesis of trans-4-amino-N-(4-chloropyridin-3-yl)cyclohexanecarboxamide hydrochloric acid saltusing 3-chloropyridin-4-amine (3.96 g, 30.8 mmol) as starting material.

LC-MS (Method 2): Rₜ=0.74 min; MS (ESIPos): m/z=254.1 [M+H]⁺

### Intermediate I11

### ({trans-4-[(2-chloro-4-fluorophenyl)carbamoyl]cyclohexyl}amino)(oxo)acetyl chloride

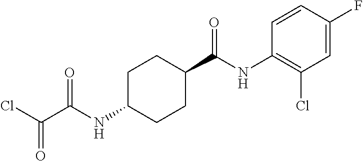 [SMILES: O=C(Cl)C(=O)N[C@H]1CC[C@H](C(=O)Nc2ccc(F)cc2Cl)CC1]

To a suspension of 2.05 g (7.57 mmol) trans-4-amino-N-(2-chloro-4-fluorophenyl) cyclohexanecarboxamide in 200 ml dichloromethane were added 60 ml (121 mmol) ethanedioyl dichloride and the mixture was stirred at room temperature for 3 hours. The reaction mixture was then concentrated in vacuo to give 2.72 mg of the title compound as a solid material which was used without further purification.

### Intermediate I12

### methyl 5-({trans-4-[(2-chloro-4-fluorophenyl)carbamoyl]cyclohexyl}carbamoyl)-1,3-oxazole-4-carboxylate

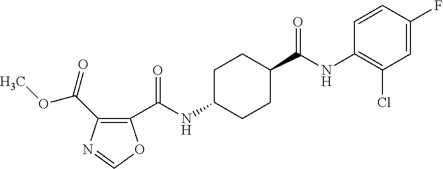 [SMILES: COC(=O)c1ncoc1C(=O)N[C@H]1CC[C@H](C(=O)Nc2ccc(F)cc2Cl)CC1]

To a suspension of 2.72 g (7.53 mmol) ({trans-4-[(2-chloro-4-fluorophenyl)carbamoyl]cyclohexyl}amino)(oxo)acetyl chloride in 60 ml dioxane, 615 mg (9.04 mmol) imidazole, 3.41 ml (24.5 mmol) triethylamine and 721 μl (7.53 mmol) methyl isocyanoacetate were subsequently added and the mixture was stirred for 48 hours at 90° C. The reaction mixture was concentrated in vacuo and the residue was purified by flash column chromatography (three times) to give 186 mg of the title compound as a solid material.

LCMS (Method 1): Rₜ=1.08 min; MS (ESIpos) m/z=424.0 [M+H]⁺.

### Intermediate I13

### trans-N-(2-chloro-4-fluorophenyl)-4-[(isocyanoacetyl)amino] cyclohexane-carboxamide

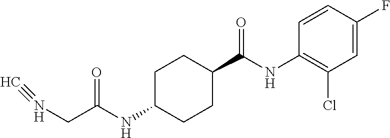

To a suspension of 1.50 g (4.88 mmol) trans-4-amino-N-(2-chloro-4-fluorophenyl)cyclohexanecarboxamide and 685 mg (5.56 mmol) potassium isocyanoacetate in 45 ml abs DMF were added 2.6 ml (14.6 mmol)N-ethyl-N-isopropylpropan-2-amine and 2.2 g (5.76 mmol) HATU and the reaction was stirred at room temperature for 5 hours. The reaction mixture was poured into water and the mixture was extracted three times with dichloromethane. The combined organic phases were washed with saturated sodiumbicarbonate solution until a neutral pH was reached. The organic phase was dried over sodium sulphate, and the solvent was removed under reduced pressure to give 1.54 g of the crude material which was used in subsequent steps without further purification.

### Intermediate I14

### methyl 4-({trans-4-[(2-chloro-4-fluorophenyl)carbamoyl]cyclohexyl}carbamoyl)-1,3-oxazole-5-carboxylate

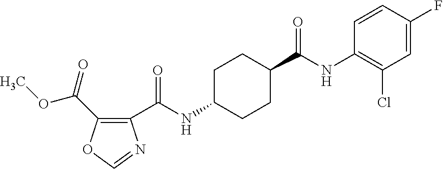 [SMILES: COC(=O)c1ocnc1C(=O)N[C@H]1CC[C@H](C(=O)Nc2ccc(F)cc2Cl)CC1]

To a suspension of 1.65 g (4.89 mmol) trans-N-(2-chloro-4-fluorophenyl)-4-[(isocyanoacetyl)amino] cyclohexane-carboxamide in 40 ml abs. dioxane, 399 mg (5.86 mmol) imidazole, 2.21 ml (15.9 mmol) triethylamine and 468 μl (4.89 mmol) methyl chloro(oxo)acetate were subsequently added and the mixture was stirred for 8 hours at 90° C. The reaction mixture was concentrated in vacuo and the residue was purified by flash column chromatography to give 210 mg of the title compound as a solid material.

LCMS (Method 1): Rₜ=1.01 min; MS (ESIpos) m/z=424.0 [M+H]⁺.

### Intermediate I15

### Methyl (1α,3β,4β)-4-[(tert-butoxycarbonyl)amino]-3-fluorocyclohexanecarboxylate

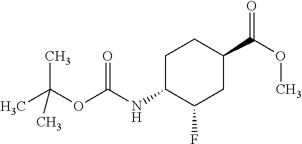 [SMILES: COC(=O)[C@@H]1CC[C@@H](NC(=O)OC(C)(C)C)[C@@H](F)C1]

To a suspension of 400 mg (1.46 mmol) methyl (1α,3α,4β)-4-[(tert-butoxycarbonyl)amino]-3-hydroxycyclohexanecarboxylate (for preparation see: I. Z. Siemion et al. Tetrahedron: Asymmetry 2001, 12, 455) in 10 ml abs. tetrahydrofuran were added dropwise 350 μl (2.34 mmol) DBU at 0° C. 410 μl (2.19 mmol) 1,1,2,2,3,3,4,4,4-nonafluorobutane-1-sulfonyl fluoride were added and the mixture was stirred at 0° C. for 3 hours. The reaction mixture was poured into saturated sodiumbicarbonat solution and the mixture was extracted with dichloromethane. The combined organic phases were washed with saturated sodiumbicarbonate solution until a neutral pH was reached. The organic phase was dried over sodium sulphate, and the solvent was removed under reduced pressure to give 450 mg of the crude material which was used in subsequent steps without further purification.

### Intermediate I16

### methyl (1α,3β,4β)-4-amino-3-fluorocyclohexanecarboxylate

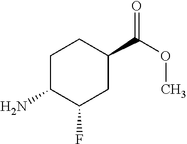

To a suspension of 450 mg (1.63 mmol) methyl (1α,3β,4β)-4-[(tert-butoxycarbonyl)amino]-3-fluorocyclohexanecarboxylate in 5 ml dichloromethane were added 4.5 ml (58.8 mmol) trifluoro acetic acid and the mixture was stirred at room temperature for 24 hours. The reaction mixture was concentrated in vacuo and the residue was treated with saturated sodiumbicarbonate solution. The mixture was extracted with a mixture of dichloromethane/methanol (8:2). The combined organic phases were washed with saturated sodiumbicarbonate solution until a neutral pH was reached. The organic phase was dried over sodium sulphate, and the solvent was removed under reduced pressure to give 301 mg of the crude material which was used in subsequent steps without further purification.

### Intermediate I17

### methyl(1α,3β,4β)-3-fluoro-4-({[4-(methylcarbamoyl)-1H-imidazol-5-yl]carbonyl}amino)cyclohexanecarboxylate

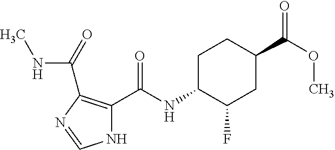

To a suspension of 301 mg (1.72 mmol) methyl (1α,3β,4β)-4-amino-3-fluorocyclohexanecarboxylate and 479 μl (3.44 mmol) triethylamine in 18 ml dichloromethane were added 371 mg (0.86 mmol) N,N-dimethyl-5,10-dioxo-5H,10H-diimidazo[1,5-a:1′,5′-d]pyrazine-1,6-dicarboxamide and the mixture was stirred for 24 hours at room temperature. The reaction mixture was concentrated in vacuo and the residue was purified by flash column chromatography to give 240 mg of the title compound as a solid material which was used in the subsequent steps without further purification.

LCMS (Method 1): Rₜ=0.84 min; MS (ESIpos) m/z=327.1 [M+H]⁺.

### Intermediate I18

### methyl (trans)-4-[(tert-butoxycarbonyl)amino]-3-oxocyclohexanecarboxylate

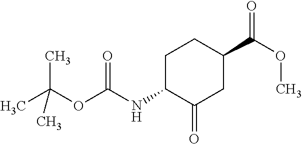 [SMILES: COC(=O)[C@@H]1CC[C@@H](NC(=O)OC(C)(C)C)C(=O)C1]

To a suspension of 400 mg (1.46 mmol) (1α,3α,4β)-4-[(tert-butoxycarbonyl)amino]-3-hydroxycyclohexanecarboxylate (for preparation see: I. Z. Siemion et al. Tetrahedron: Asymmetry 2001, 12, 455) in 20 ml abs. DMF 3.30 g (8.78 mmol) pryidiniumdichromate were added in portions and the mixture was stirred at room temperature for 48 hours. The reaction mixture was poured into water and the aqueous phase was extracted with diethyl ether. The combined organic phases were washed with brine and the organic phase was dried over sodium sulphate. The solvent was removed under reduced pressure to give 450 mg of the crude material which was used in subsequent steps without further purification.

### Intermediate I19

### Methyl(trans)-4-[(tert-butoxycarbonyl)amino]-3,3-difluorocyclohexanecarboxylate

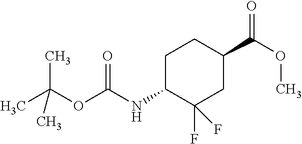 [SMILES: COC(=O)[C@@H]1CC[C@@H](NC(=O)OC(C)(C)C)C(F)(F)C1]

To a suspension of 450 mg (1.66 mmol) methyl (trans)-4-[(tert-butoxycarbonyl)amino]-3-oxocyclohexanecarboxylate in 14 ml dichloromethane a solution of 263 μl (1.99 mmol) DAST in 1 ml dichloromethane were added and the mixture was stirred at room temperature for 24 hours. The reaction mixture was poured into saturated sodiumbicarbonat solution and the aqueous phase was extracted with dichloromethane. The combined organic phases were washed with saturated sodiumbicarbonate solution until a neutral pH was reached. The organic phase was dried over sodium sulphate, and the solvent was removed under reduced pressure to give 510 mg of the crude material which was used in subsequent steps without further purification.

### Intermediate I20

### methyl (trans)-4-amino-3,3-difluorocyclohexanecarboxylate

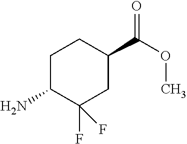

To a suspension of 510 mg (1.74 mmol) methyl (trans)-4-[(tert-butoxycarbonyl)amino]-3,3-difluorocyclohexanecarboxylate in 5 ml dichloromethane were added 4.8 ml (62.6 mmol) trifluoro acetic acid and the mixture was stirred at room temperature for 24 hours. The reaction mixture was concentrated in vacuo and the residue was treated with saturated sodiumbicarbonate solution. The aqueous phase was extracted with a mixture of dichloromethane/methanol (8:2). The combined organic phases were washed with saturated sodiumbicarbonate solution until a neutral pH was reached. The organic phase was dried over sodium sulphate, and the solvent was removed under reduced pressure to give 300 mg of the crude material which was used in subsequent steps without further purification.

### Intermediate I21

### Methyl (trans)-3,3-difluoro-4-({[4-(methylcarbamoyl)-1H-imidazol-5-yl]carbonyl}amino)cyclohexanecarboxylate

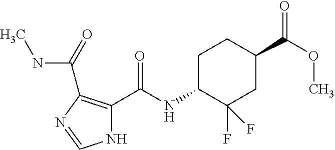

To a suspension of 300 mg (1.55 mmol) methyl (trans)-4-amino-3,3-difluorocyclohexanecarboxylate and 433 μl (3.11 mmol) triethylamine in 17 ml dichloromethane were added 335 mg (0.78 mmol) N,N-dimethyl-5,10-dioxo-5H,10H-diimidazo[1,5-a:1′,5′-d]pyrazine-1,6-dicarboxamide and the mixture was stirred for 24 hours at room temperature. The reaction mixture was concentrated in vacuo and the residue was purified by flash column chromatography to give 200 mg of the title compound as a solid material which was used in the subsequent steps without further purification.

LCMS (Method 1): Rₜ=0.86 min; MS (ESIpos) m/z=345.1 [M+H]⁺.

### Intermediate I22

### Methyl (1α,3α,4β)-4-[(tert-butoxycarbonyl)amino]-3-methoxycyclohexanecarboxylate

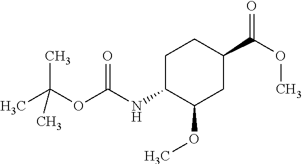 [SMILES: COC(=O)[C@@H]1CC[C@@H](NC(=O)OC(C)(C)C)[C@H](OC)C1]

To a suspension 400 mg (1.46 mmol) (1α,3α,4β)-4-[(tert-butoxycarbonyl)amino]-3-hydroxycyclohexanecarboxylate (for preparation see: I. Z. Siemion et al. Tetrahedron: Asymmetry 2001, 12, 455) in 6 ml dichloromethane were added 1.01 g (4.39 mmol) silver(I)oxide, 273 μl (4.39 mmol) iodomethane and 400 mg molecular sieve. The mixture was stirred at room temperature for 3 days. The solids were filtered off and the filtrate was concentrated under reduced pressure to give 420 mg of the crude material which was used in subsequent steps without further purification.

### Intermediate I23

### methyl (1α,3α,4β)-4-amino-3-methoxycyclohexanecarboxylate

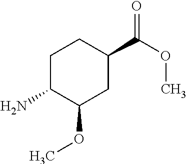

To a suspension of 420 mg (1.46 mmol) methyl (1α,3α,4β)-4-[(tert-butoxycarbonyl)amino]-3-methoxycyclohexanecarboxylate in 5 ml dichloromethane were added 4.0 ml (52.6 mmol) trifluoroactic acid and the mixture was stirred at room temperature for 24 hours. The reaction mixture was concentrated in vacuo and the residue was treated with saturated sodiumbicarbonate solution. The aqueous phase was extracted with a mixture of dichloromethane/methanol (8:2). The combined organic phases were washed with saturated sodiumbicarbonate solution until a neutral pH was reached. The organic phase was dried over sodium sulphate, and the solvent was removed under reduced pressure to give 264 mg of the crude material which was used in subsequent steps without further purification.

### Intermediate I24

### methyl(1α,3α,4β)-3-methoxy-4-({[4-(methylcarbamoyl)-1H-imidazol-5-yl]carbonyl}amino)cyclohexanecarboxylate

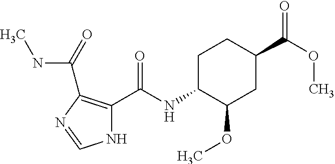

To a suspension of 264 mg (1.41 mmol) methyl (1α,3α,4β)-4-amino-3-methoxycyclohexanecarboxylate and 393 μl (2.82 mmol) triethylamine in 15 ml dichloromethane were added 304 mg (0.70 mmol) N,N-dimethyl-5,10-dioxo-5H,10H-diimidazo[1,5-a:1′,5′-d]pyrazine-1,6-dicarboxamide and the mixture was stirred for 3 days at room temperature. The reaction mixture was concentrated in vacuo and the residue was purified by flash column chromatography to give 210 mg of the title compound as a solid material which was used in the subsequent steps without further purification.

LCMS (Method 1): Rₜ=0.80 min; MS (ESIpos) m/z=339.1 [M+H]⁺.

### Intermediate I25

### phenyl 5-({trans-4-[(2-chloro-4-fluoro-5-methylphenyl)carbamoyl]cyclohexyl}carbamoyl)-1H-imidazole-4-carboxylate

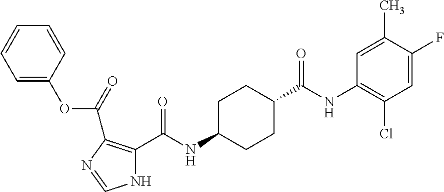 [SMILES: Cc1cc(NC(=O)[C@H]2CC[C@H](NC(=O)c3[nH]cnc3C(=O)Oc3ccccc3)CC2)c(Cl)cc1F]

1-Chloro-N,N,2-trimethylprop-1-en-1-amine (748 mg, 5.60 mmol) was added to a solution of trans-4-({[4-(phenoxycarbonyl)-1H-imidazol-5-yl]carbonyl}amino)cyclohexanecarboxylic acid (1.00 g, 2.80 mmol) in dichloromethane (63 ml) and the mixture was stirred at room temperature for 30 min. Pyridine (0.68 ml, 8.4 mmol) and 2-chloro-4-fluoro-5-methylaniline (491 mg, 3.08 mmol, CAS No. 124185-35-9) were added an the mixture was stirred over night at room temperature. For work-up, water was added, the mixture was extracted with ethyl acetate and the organic phase was washed with a saturated sodium bicarbonate solution and water. The organic phase was dried by filtration through a silicone filter and the filtrate was concentrated. The crude product was stirred with methanol. The precipitate was collected by filtration, washed with methanol and dried to yield the title compound (445 mg, 32% yield).

LC-MS (Method 6): Rₜ=1.18 min; MS (ESIpos) m/z=499.1 [M+H]⁺.

¹H-NMR (400 MHz, DMSO-d₆): δ [ppm]=13.57 (br. s., 1H), 9.46-9.30 (m, 2H), 7.94 (s, 1H), 7.54-7.43 (m, 3H), 7.43-7.24 (m, 4H), 3.80-3.65 (m, 1H), 2.44-2.35 (m, 1H), 2.20 (d, 3H), 2.06-1.82 (m, 4H), 1.63-1.19 (m, 4H).

### Intermediate I26

### phenyl 5-({trans-4-[(2-chloro-4,6-difluorophenyl)carbamoyl]cyclohexyl}carbamoyl)-1H-imidazole-4-carboxylate

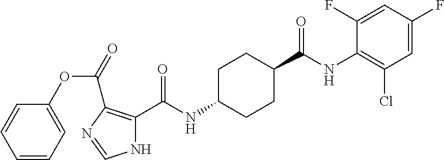 [SMILES: O=C(Oc1ccccc1)c1nc[nH]c1C(=O)N[C@H]1CC[C@H](C(=O)Nc2c(F)cc(F)cc2Cl)CC1]

1-Chloro-N,N,2-trimethylprop-1-en-1-amine (782 mg, 5.86 mmol) was added to a solution of trans-4-({[4-(phenoxycarbonyl)-1H-imidazol-5-yl]carbonyl}amino)cyclohexanecarboxylic acid (1.29 g, 3.36 mmol) in dichloromethane (75 ml) and the mixture was stirred at room temperature for 30 min. Pyridine (0.82 ml, 10 mmol) and 2-chloro-4,6-difluoroaniline (550 mg, 3.36 mmol, CAS No. 36556-56-6) were added and the mixture was stirred over night at room temperature. For work-up, water was added, the mixture was extracted with ethyl acetate and the organic phase was washed with saturated sodium bicarbonate solution and water. The organic phase was dried by filtration through a silicone filter and the filtrate was concentrated. The crude product was purified by flash chromatography (50 g Snap-Catridge, hexane/ethyl acetate gradient 50%->100% ethyl acetate) to yield the title compound (1.05 g, 61% yield).

LC-MS (Method 2): Rₜ=0.93 min; MS (ESIpos) m/z=503.3 [M+H]⁺.

¹H-NMR (400 MHz, DMSO-d₆): δ [ppm]=13.54 (br. s, 1H), 9.53 (s, 1H), 9.43-9.10 (m, 1H), 7.92 (s, 1H), 7.53-7.22 (m, 7H), 3.80-3.65 (m, 1H), 2.40-2.29 (m, 1H), 2.04-1.83 (m, 4H), 1.59-1.44 (m, 2H), 1.35-1.21 (m, 2H).

### Intermediate I27

### phenyl 5-({trans-4-[(5-chloropyrimidin-4-yl)carbamoyl]cyclohexyl}carbamoyl)-1H-imidazole-4-carboxylate

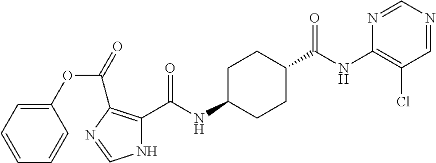 [SMILES: O=C(Oc1ccccc1)c1nc[nH]c1C(=O)N[C@H]1CC[C@H](C(=O)Nc2ncncc2Cl)CC1]

1-Chloro-N,N,2-trimethylprop-1-en-1-amine (500 μl, 3.8 mmol) was added to a solution of trans-4-({[4-(phenoxycarbonyl)-1H-imidazol-5-yl]carbonyl}amino)cyclohexanecarboxylic acid (672 mg, 1.88 mmol) in dichloromethane (45 ml) and the mixture was stirred at room temperature for 1.5 h. Pyridine (460 μl, 5.6 mmol) and 5-chloropyrimidin-4-amine (244 mg, 1.88 mmol, CAS No. 101257-82-3) were added and the mixture was stirred over night at room temperature. For work-up, water was added, the mixture was extracted with ethyl acetate and the organic phase was washed with saturated sodium bicarbonate solution and water. The organic phase was dried by filtration through a silicone filter and the filtrate was concentrated. The crude product purified by flash chromatography (25 g Snap-Catridge, ethyl acetate/ethanol gradient 0%->5% ethanol) to yield the title compound (140 mg, 16% yield).

LC-MS (Method 2): Rₜ=0.68 min; MS (ESIPos): m/z=469.3 [M+H]⁺

¹H-NMR (400 MHz, DMSO-d₆): δ [ppm]=13.57 (br. s., 1H), 10.48 (br. s., 1H), 9.35 (br. s., 1H), 8.92 (s, 1H), 8.84 (s, 1H), 7.93 (s, 1H), 7.52-7.43 (m, 2H), 7.37-7.25 (m, 3H), 3.73 (d, 1H), 2.07-1.84 (m, 4H), 1.59-1.44 (m, 2H), 1.34-1.15 (m, 2H).

### Intermediate I28

### phenyl 5-({trans-4-[(2-chloro-4,5-difluorophenyl)carbamoyl]cyclohexyl}carbamoyl)-1H-imidazole-4-carboxylate

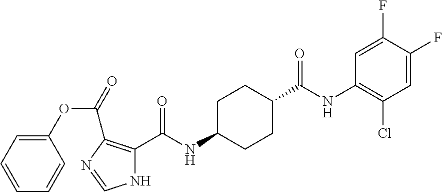 [SMILES: O=C(Oc1ccccc1)c1nc[nH]c1C(=O)N[C@H]1CC[C@H](C(=O)Nc2cc(F)c(F)cc2Cl)CC1]

Was prepared in analogy to the synthesis of phenyl 5-({trans-4-[(5-chloropyrimidin-4-yl)carbamoyl]cyclohexyl}carbamoyl)-1H-imidazole-4-carboxylate using 2-chloro-4,5-difluoroaniline (1.00 g, 6.11 mmol, CAS No 2613-32-3) as starting material. The crude product was stirred with methanol and the precipitate formed was collected by filtration, washed with methanol and dried to give the title compound (1.14 g, 37% yield).

¹H-NMR (400 MHz, DMSO-d₆): δ [ppm]=13.55 (br. s., 1H), 9.53 (s, 1H), 9.45-9.10 (m, 1H), 7.93 (s, 1H), 7.82-7.72 (m, 2H), 7.52-7.44 (m, 2H), 7.36-7.23 (m, 3H), 3.80-3.65 (m, 1H), 2.47-2.39 (m, 1H), 2.05-1.94 (m, 2H), 1.93-1.84 (m, 2H), 1.59-1.43 (m, 2H), 1.33-1.20 (m, 2H).

### Intermediate I29

### phenyl 5-[(trans-4-{[2-chloro-5-(morpholin-4-yl)phenyl]carbamoyl}cyclohexyl)carbamoyl]-1H-imidazole-4-carboxylate

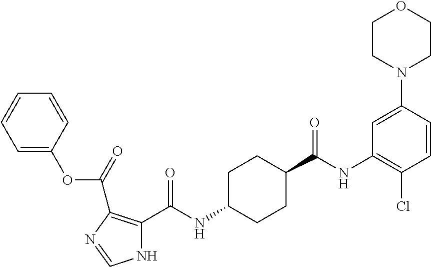 [SMILES: O=C(Oc1ccccc1)c1nc[nH]c1C(=O)N[C@H]1CC[C@H](C(=O)Nc2cc(N3CCOCC3)ccc2Cl)CC1]

1-Chloro-N,N,2-trimethylprop-1-en-1-amine (748 mg, 5.60 mmol) was added to a solution of trans-4-({[4-(phenoxycarbonyl)-1H-imidazol-5-yl]carbonyl}amino)cyclohexanecarboxylic acid 1.00 g, 2.80 mmol) in dichloromethane (63 ml) and the mixture was stirred at room temperature for 30 min. Pyridine (680 μl, 8.4 mmol) and 2-chloro-5-(morpholin-4-yl)aniline (655 mg, 3.08 mmol, CAS No. 915921-20-9) were added and the mixture was stirred over night at room temperature. For work-up, the reaction mixture was concentrated, water was added and the mixture was extracted with ethyl acetate. Insoluble material was collected by filtration and washed with methanol and dried to give the title compound (1.22 g, 76% yield). The ethyl acetate phase was washed with brine, filtrated through a phase separator and concentrated. The residue was stirred with methanol and the precipitate formed was collected by filtration, washed with methanol and dried to give a second fraction of the title compound (0.35 g, 21% yield).

LC-MS (Method 2): Rₜ=0.96 min; MS (ESIpos): m/z=552.4 [M+H]⁺

¹H-NMR (400 MHz, DMSO-d₆): δ [ppm]=13.58 (br. s, 1H), 9.36 (d, 1H), 9.25 (s, 1H), 7.94 (br. s, 1H), 7.52-7.41 (m, 2H), 7.36-7.05 (m, 5H), 6.77 (dd, 1H), 3.81-3.64 (m, 5H), 3.10-2.98 (m, 4H), 2.15-1.74 (m, 4H), 1.62-1.09 (m, 4H).

### Intermediate I30

### phenyl 5-({trans-4-[(4-chloropyridin-3-yl)carbamoyl]cyclohexyl}carbamoyl)-1H-imidazole-4-carboxylate

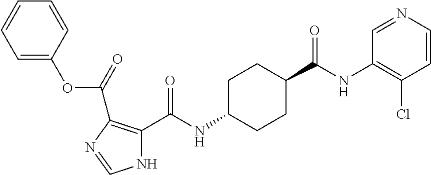 [SMILES: O=C(Oc1ccccc1)c1nc[nH]c1C(=O)N[C@H]1CC[C@H](C(=O)Nc2cnccc2Cl)CC1]

Was prepared in analogy to the synthesis of phenyl 5-[(trans-4-{[2-chloro-5-(morpholin-4-yl)phenyl]carbamoyl}cyclohexyl)carbamoyl]-1H-imidazole-4-carboxylate using 4-chloropyridin-3-amine (396 mg, 3.08 mmol, Cas No. 20511-15-3) as starting material. For work-up, water was added and the mixture was extracted with dichloromethane. The combined organic phases were washed with brine and filtrated through a phase separator. Upon concentration under reduced pressure, a precipitate was formed, which was collected by filtration, washed with methanol and dried to give the title compound (390 mg, 94% purity, 28% yield).

LC-MS (Method 2): Rₜ=0.80 min; MS (ESIpos): m/z=468.3 [M+H]⁺

¹H-NMR (400 MHz, DMSO-d₆): δ [ppm]=9.71 (s, 1H), 9.37 (br. s., 1H), 8.70 (s, 1H), 8.33 (d, 1H), 7.94 (s, 1H), 7.60 (d, 1H), 7.52-7.44 (m, 2H), 7.37-7.21 (m, 3H), 3.79-3.67 (m, 1H), 2.48-2.37 (m, 1H), 2.06-1.85 (m, 4H), 1.61-1.45 (m, 2H), 1.33-1.20 (m, 2H).

### Intermediate I31

### ethyl 5-methyl-7,8-dihydro-6H-cyclopenta[e]pyrazolo[1,5-a]pyrimidine-3-carboxylate (major isomer) and ethyl 8-methyl-6,7-dihydro-5H-cyclopenta[d]pyrazolo[1,5-a]pyrimidine-3-carboxylate (minor isomer)

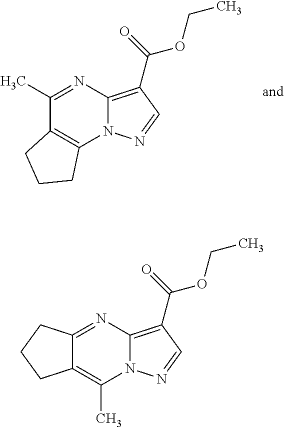 [SMILES: CCOC(=O)c1cnn2c3c(c(C)nc12)CCC3 ; CCOC(=O)c1cnn2c(C)c3c(nc12)CCC3]

A mixture of ethyl 5-amino-1H-pyrazole-4-carboxylate (1.00 g, 6.45 mmol) and 2-acetylcyclopentanone (1.63 g, 12.9 mmol, CAS No. 1670-46-8) in acetic acid (5.0 ml) was heated for 1 h at 110° C. in a microwave reactor (Biotage Initator). Upon cooling to room temperature, the reaction mixture was portioned between water and dichlormethane and the organic phase was washed with water, filtrated through a silicone filter and concentrated under vacuum. The crude product was purified by flash chromatography (50 g Snap Cartrigde, hexanes/ethyl acetate gradient, 12%->100% ethyl acetate) to yield the title compound ethyl 5-methyl-7,8-dihydro-6H-cyclopenta[e]pyrazolo[1,5-a]pyrimidine-3-carboxylate (major isomer) as mixture with the isomer ethyl 8-methyl-6,7-dihydro-5H-cyclopenta[d]pyrazolo[1,5-a]pyrimidine-3-carboxylate (ca. 85/15) (1.28 g, 81% yield for both isomers).

### Major Isomer:

LC-MS (Method 2): Rₜ=0.94 min; MS (ESIpos) m/z=246.1 [M+H]⁺.

¹H-NMR (400 MHz, DMSO-d₆): δ [ppm]=8.53 (s, 1H), 4.28 (q, 2H), 3.08-3.00 (m, 2H), 2.56 (s, 3H), 2.31-2.20 (m, 2H), 1.37-1.26 (m, 3H).

### Minor Isomer (Characteristic Signals Given):

¹H-NMR (400 MHz, DMSO-d₆): δ [ppm]=8.50 (s, 1H), 3.01-2.95 (m, 2H), 2.69 (s, 3H), 2.21-2.14 (m, 2H).

### Intermediate I32

### 5-methyl-7,8-dihydro-6H-cyclopenta[e]pyrazolo[1,5-a]pyrimidine-3-carboxylic acid (major isomer) and 8-methyl-6,7-dihydro-5H-cyclopenta[d]pyrazolo[1,5-a]pyrimidine-3-carboxylic acid (minor isomer)

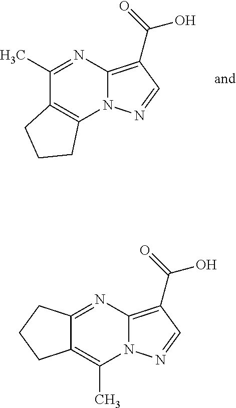 [SMILES: Cc1nc2c(C(=O)O)cnn2c2c1CCC2 ; Cc1c2c(nc3c(C(=O)O)cnn13)CCC2]

A mixture of ethyl 5-methyl-7,8-dihydro-6H-cyclopenta[e]pyrazolo[1,5-a]pyrimidine-3-carboxylate (major isomer) and ethyl 8-methyl-6,7-dihydro-5H-cyclopenta[d]pyrazolo[1,5-a]pyrimidine-3-carboxylate (minor isomer) (1.28 g, 5.22 mmol, ratio of isomers ca 85/15) was stirred in a mixture of lithium hydroxide (26 ml, 26 mmol, 1 M aqueous solution), tetrahydrofuran (43 ml) and methanol (9.1 ml) for 3 days at room temperature. For work-up, aqueous hydrochloric acid (2 M) was added and the precipitate formed was collected by filtration, washed with water and dried to yield the title compounds as mixture of isomers (ca 10:1) (770 mg, 68%).

### Major Isomer:

LC-MS (Method 1): Rₜ=0.71 min; MS (ESIpos) m/z=218.1 [M+H]⁺.

¹H-NMR (400 MHz, DMSO-d₆): δ [ppm]=12.83-11.51 (br. s, 1H), 8.48 (s, 1H), 3.03 (t, 2H), 2.55 (s, 3H), 2.30-2.21 (m, 2H).

### Minor Isomer (Characteristic Signals Given):

¹H-NMR (400 MHz, DMSO-d₆): δ [ppm]=8.45 (s, 1H), 3.00-2.95 (m, 2H), 2.68 (s, 3H), 2.20-2.10 (m, 2H).

### Intermediate I33

### ethyl 5-(methoxymethyl)-7-methylpyrazolo[1,5-a]pyrimidine-3-carboxylate (isomer 1) and ethyl 7-(methoxymethyl)-5-methylpyrazolo[1,5-a]pyrimidine-3-carboxylate (isomer 2)

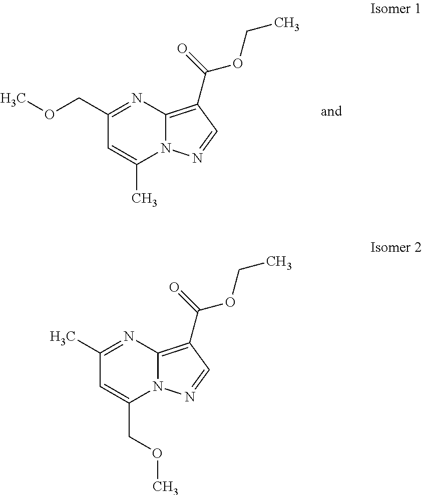

A mixture of ethyl 5-amino-1H-pyrazole-4-carboxylate (1.00 g, 6.45 mmol) and 1-methoxypentane-2,4-dione (1.68 g, 12.9 mmol, CAS No. 6290-50-2) in acetic acid (5.0 ml) was heated for 1 h at 1100 in a microwave reactor (Biotage Initator). Upon cooling to room temperature, the reaction mixture was portioned between water and dichlormethane and the organic phase was washed with water, filtrated through a silicone filter and concentrated under vacuum. The crude product was purified by flash chromatography (50 g Snap Cartrigde, hexanes/ethyl acetate 1:1) to yield the title compound as mixture of isomers fraction 1 (1.35 g, 5.42 mmol, 1:1 mixture of isomer1/isomer 2) and fraction 2 (80 mg, 0.32 mmol, 85:15 mixture of isomer 1/isomer 2).

### Fraction 2:

LC-MS (Method 2): Rₜ=0.89 min (Isomer 1); 0.91 min (isomer 2);

¹H-NMR (400 MHz, DMSO-d₆ isomer 1): δ [ppm]=8.62 (s, 1H), 7.31-7.26 (m, 1H), 4.61 (s, 2H), 4.33-4.25 (m, 2H), 3.44 (s, 3H), 2.80 (d, 3H), 1.31 (t, 3H).

¹H-NMR (400 MHz, DMSO-d₆ isomer 2, characteristic signals): δ [ppm]=8.63 (s, 1H), 7.61 (s, 1H), 4.01 (s, 3H), 2.68 (s, 3H).

### Intermediate I34

### 5-(methoxymethyl)-7-methylpyrazolo[1,5-a]pyrimidine-3-carboxylic acid (isomer 1) and 7-(methoxymethyl)-5-methylpyrazolo[1,5-a]pyrimidine-3-carboxylic acid (isomer 2)

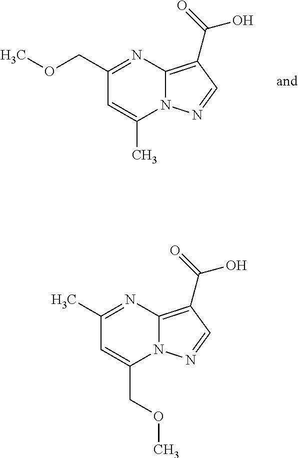 [SMILES: COCc1cc(C)n2ncc(C(=O)O)c2n1 ; COCc1cc(C)nc2c(C(=O)O)cnn12]

A mixture of ethyl 5-(methoxymethyl)-7-methylpyrazolo[1,5-a]pyrimidine-3-carboxylate (isomer 1) and ethyl 7-(methoxymethyl)-5-methylpyrazolo[1,5-a]pyrimidine-3-carboxylate (isomer 2) (1.35 g, 5.42 mmol, ratio of isomers ca 1:1) was stirred in a mixture of lithium hydroxide (27 ml, 27 mmol, 1 M aqueous solution), tetrahydrofuran (35 ml) and methanol (9.4 ml) for 3 days at room temperature. For work-up, aqueous hydrochloric acid (2 M) was added and the precipitate formed was collected by filtration. The filtrated was extracted with ethyl acetate (3×), washed with brine, filtrated through a silicon filter and concentrated to give the title compounds as mixture of isomers (ca 1:1) (820 mg, 69%) which was used in the next step without further purification.

### Intermediate I35

### ethyl 7-tert-butyl-5-methylpyrazolo[1,5-a]pyrimidine-3-carboxylate

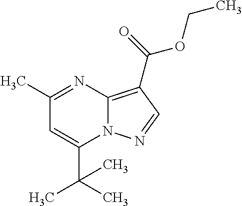 [SMILES: CCOC(=O)c1cnn2c(C(C)(C)C)cc(C)nc12]

A mixture of ethyl 5-amino-1H-pyrazole-4-carboxylate (546 mg, 3.52 mmol) and 5,5-dimethylhexane-2,4-dione (1.03 g, 97% purity, 7.03 mmol) in acetic acid (2.7 ml) was heated for 1 h at 110° C. in a microwave reactor (Biotage Initator). Upon cooling to room temperature, the reaction mixture was portioned between water and dichlormethane and the organic phase was washed with water, filtrated through a silicone filter and concentrated under vacuum. The crude product was purified by flash chromatography (25 g Snap Cartrigde, hexanes/ethyl acetate gradient) to yield the title compound (850 mg, 93% yield).

LC-MS (Method 1): Rₜ=1.25 min; MS (ESIpos) m/z=262.2 [M+H]⁺.

1H-NMR (400 MHz, DMSO-d6) delta [ppm]: 1.281 (2.05), 1.299 (4.51), 1.316 (2.11), 1.375 (0.78), 1.558 (16.00), 2.619 (8.39), 4.252 (0.66), 4.270 (2.08), 4.288 (2.05), 4.305 (0.61), 7.072 (2.38), 8.569 (2.97).

### Intermediate I36

### 7-tert-butyl-5-methylpyrazolo[1,5-a]pyrimidine-3-carboxylic acid

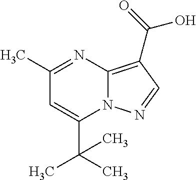 [SMILES: Cc1cc(C(C)(C)C)n2ncc(C(=O)O)c2n1]

A mixture of ethyl 7-tert-butyl-5-methylpyrazolo[1,5-a]pyrimidine-3-carboxylate (320 μl, 1.8 mmol) and lithium hydroxide (9.0 ml, 1 M aqueous solution, 9.0 mmol), tetrahydrofuran (12 ml) and methanol (3.1 ml) was stirred at room temperature for 3 days. The mixture was concentrated under reduced pressure, acidified by addition of hydrochloric acid (2M) and extracted with ethyl acetate (3×). The combined organic phases were washed with brine, filtrated through a silicon filter and dried to give the title compound (/308 mg, 72% yield).

LC-MS (Method 2): Rₜ=0.56 min; MS (ESIneg): m/z=232 [M−H]⁻

1H-NMR (400 MHz, DMSO-d6) delta [ppm]: 1.558 (16.00), 2.610 (8.22), 7.044 (2.36), 8.533 (2.91).

### Intermediate I37

### ethyl 7-cyclopropyl-5-methylpyrazolo[1,5-a]pyrimidine-3-carboxylate and ethyl 5-cyclopropyl-7-methylpyrazolo[1,5-a]pyrimidine-3-carboxylate

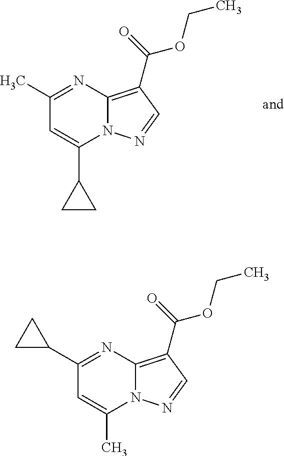 [SMILES: CCOC(=O)c1cnn2c(C3CC3)cc(C)nc12 ; CCOC(=O)c1cnn2c(C)cc(C3CC3)nc12]

Was prepared in analogy to the synthesis of ethyl 7-tert-butyl-5-methylpyrazolo[1,5-a]pyrimidine-3-carboxylate using 1-cyclopropylbutane-1,3-dione (910 μl, 7.9 mmol) as starting material. The crude product was purified by flash chromatography (25 g Snap Cartdrige, hexanes/ethyl acetate gradient, 12%->50% ethyl acetate) to give ethyl 7-cyclopropyl-5-methylpyrazolo[1,5-a]pyrimidine-3-carboxylate (549 mg, 55% yield) and ethyl 5-cyclopropyl-7-methylpyrazolo[1,5-a]pyrimidine-3-carboxylate (295 mg, 30% yield).

### 7-cyclopropyl-5-methylpyrazolo[1,5-a]pyrimidine-3-carboxylate

LC-MS (Method 1): Rₜ=0.97 min; MS (ESIpos): m/z=246.1 [M+H]⁺

1H-NMR (400 MHz, DMSO-d6) delta [ppm]: 1.135 (0.40), 1.147 (1.51), 1.153 (1.52), 1.159 (1.44), 1.166 (1.47), 1.176 (0.64), 1.285 (4.25), 1.293 (1.60), 1.300 (1.78), 1.303 (10.10), 1.314 (1.75), 1.321 (5.49), 1.332 (0.52), 2.523 (0.66), 2.539 (16.00), 2.778 (0.47), 2.786 (0.51), 2.799 (0.94), 2.812 (0.48), 2.820 (0.45), 4.245 (1.14), 4.263 (3.75), 4.280 (3.72), 4.298 (1.13), 6.845 (4.22), 8.554 (5.58).

### ethyl 5-cyclopropyl-7-methylpyrazolo[1,5-a]pyrimidine-3-carboxylate

LC-MS (Method 1): Rₜ=1.04 min; MS (ESIpos): m/z=246.1 [M+H]⁺

1H-NMR (400 MHz, DMSO-d6) delta [ppm]: 1.105 (1.33), 1.111 (4.14), 1.116 (3.07), 1.120 (3.11), 1.123 (8.04), 1.129 (2.74), 1.138 (2.08), 1.143 (4.39), 1.150 (1.69), 1.165 (0.50), 1.286 (6.99), 1.304 (15.77), 1.322 (7.28), 2.192 (0.44), 2.205 (0.83), 2.211 (0.85), 2.217 (0.67), 2.224 (1.81), 2.232 (0.68), 2.237 (0.85), 2.243 (0.78), 2.256 (0.41), 2.523 (1.66), 2.536 (0.87), 2.701 (16.00), 2.703 (15.53), 4.219 (2.19), 4.236 (7.14), 4.254 (7.06), 4.272 (2.16), 7.134 (4.38), 7.136 (4.35), 8.500 (9.32).

### Intermediate I38

### 7-cyclopropyl-5-methylpyrazolo[1,5-a]pyrimidine-3-carboxylic acid

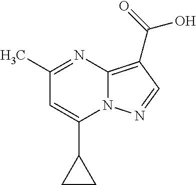 [SMILES: Cc1cc(C2CC2)n2ncc(C(=O)O)c2n1]

Was prepared in analogy to the synthesis of 7-tert-butyl-5-methylpyrazolo[1,5-a]pyrimidine-3-carboxylic acid using ethyl 7-cyclopropyl-5-methylpyrazolo[1,5-a]pyrimidine-3-carboxylate (545 mg, 2.22 mmol) as starting material. The crude product was stirred with methanol, the precipitated was filtrated off and the filtrate was concentrated and dried to give the title compound (118 mg, 47% purity by LC-MS) which was used in the next step without further purification.

LC-MS (Method 1): Rₜ=0.73 min; MS (ESIpos): m/z=218.0 [M+H]⁺

### Intermediate I39

### 5-cyclopropyl-7-methylpyrazolo[1,5-a]pyrimidine-3-carboxylic acid

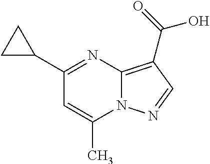 [SMILES: Cc1cc(C2CC2)nc2c(C(=O)O)cnn12]

Was prepared in analogy to the synthesis of 7-tert-butyl-5-methylpyrazolo[1,5-a]pyrimidine-3-carboxylic acid using ethyl 5-cyclopropyl-7-methylpyrazolo[1,5-a]pyrimidine-3-carboxylate (290 mg, 1.18 mmol) as starting material to give the title compound (158 mg).

LC-MS (Method 1): Rₜ=0.79 min; MS (ESIpos): m/z=218.1 [M+H]⁺

1H-NMR (400 MHz, DMSO-d6) delta [ppm]: 1.072 (0.40), 1.085 (1.72), 1.093 (3.68), 1.098 (3.49), 1.104 (3.14), 1.111 (2.75), 1.118 (3.59), 1.124 (1.64), 1.131 (1.88), 1.138 (3.45), 1.145 (1.80), 1.158 (0.58), 1.987 (0.48), 2.187 (0.44), 2.199 (0.91), 2.207 (0.90), 2.213 (0.70), 2.219 (1.71), 2.227 (0.67), 2.231 (0.90), 2.238 (0.86), 2.523 (1.57), 2.532 (0.46), 2.665 (0.42), 2.669 (0.45), 2.694 (14.96), 2.696 (16.00), 7.083 (4.38), 7.086 (4.25), 8.464 (10.72).

### Intermediate I40

### 6-(ethoxycarbonyl)pyrazolo[1,5-a]pyrimidine-3-carboxylic acid

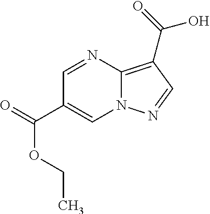 [SMILES: CCOC(=O)c1cnc2c(C(=O)O)cnn2c1]

A mixture of 5-amino-1H-pyrazole-4-carboxylic acid (1.00 g, 7.87 mmol) and ethyl 2-formyl-3-oxopropanoate (2.0 ml, 16 mmol) in acetic acid (6.0 ml) was heated for 1 h at 110° C. in a microwave reactor (Biotage Initator). Upon cooling to room temperature, the precipitate was collected by filtration, washed with water and dried under high vacuum at 50° C. to give the title compound (900 mg, 48% yield).

LC-MS (Method 1): Rₜ=0.68 min; MS (ESIpos): m/z=236.1 [M+1]⁺

1H-NMR (400 MHz, DMSO-d₆): δ [ppm]=12.59 (s, 1H), 9.72 (d, 1H), 9.13 (d, 1H), 8.76 (s, 1H), 4.41 (q, 2H), 1.38 (t, 3H).

### Intermediate I41

### ethyl 5-(4-methylpiperazin-1-yl)pyrazolo[1,5-a]pyrimidine-3-carboxylate

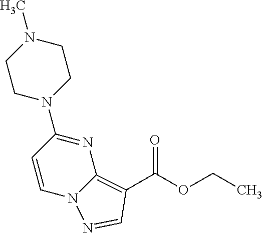 [SMILES: CCOC(=O)c1cnn2ccc(N3CCN(C)CC3)nc12]

A mixture of ethyl 5-chloropyrazolo[1,5-a]pyrimidine-3-carboxylate (677 mg, 3.00 mmol), 1-methylpiperazine (670 μl, 6.0 mmol) and N-ethyl-N-isopropylpropan-2-amine (1.6 ml, 9.0 mmol) in 2-propanol (20 ml) was refluxed for 5 h. Upon cooling, water was added and the mixture was extracted with ethyl acetate (3×). The combined organic phases were filtrated through a silicone filter and concentrated to give the title compound (830 mg) which was used in the next step without purification.

LC-MS (Method 1): Rₜ=0.57 min; MS (ESIpos): m/z=290 [M+H]⁺

¹H-NMR (400 MHz, DMSO-d₆): δ [ppm]=8.71 (d, 1H), 8.20 (s, 1H), 6.86 (d, 1H), 4.18 (q, 2H), 3.81-3.70 (m, 4H), 2.43-2.37 (m, 4H), 2.22 (s, 3H), 1.27 (t, 3H).

### Intermediate I42

### sodium 5-(4-methylpiperazin-1-yl)pyrazolo[1,5-a]pyrimidine-3-carboxylate

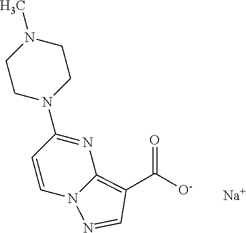 [SMILES: CN1CCN(c2ccn3ncc(C(=O)[O-])c3n2)CC1 ; [Na+]]

A mixture of ethyl 5-(4-methylpiperazin-1-yl)pyrazolo[1,5-a]pyrimidine-3-carboxylate (830 mg, 2.87 mmol), sodium hydroxide (3.6 ml, 2 M aqueous solution, 7.2 mmol), methanol (5.8 ml) and tetrahydrofuran (19 ml) was stirred for 3 h at 70° C. Upon cooling, the reaction mixture was concentrated and residue was azeotroped twice with toluene to give the title compound (1.17 g) which was used in the next step without further purification.

### Intermediate I43

### ethyl 5-(morpholin-4-yl)pyrazolo[1,5-a]pyrimidine-3-carboxylate

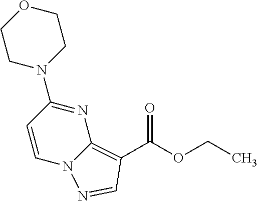 [SMILES: CCOC(=O)c1cnn2ccc(N3CCOCC3)nc12]

Was prepared in analogy to the synthesis of ethyl 5-(4-methylpiperazin-1-yl)pyrazolo[1,5-a]pyrimidine-3-carboxylate using ethyl 5-chloropyrazolo[1,5-a]pyrimidine-3-carboxylate (677 mg, 3.00 mmol) and morpholine (520 μl, 6.0 mmol) as starting materials to give the title compound (779 mg).

LC-MS (Method 1): Rₜ=0.81 min; MS (ESIpos): m/z=277 [M+H]⁺

¹H-NMR (400 MHz, DMSO-d₆): δ [ppm]=8.76 (d, 1H), 8.22 (s, 1H), 6.86 (d, 1H), 4.19 (q, 2H), 3.80-3.66 (m, 8H), 1.28 (t, 3H).

### Intermediate I44

### lithium 5-(morpholin-4-yl)pyrazolo[1,5-a]pyrimidine-3-carboxylate

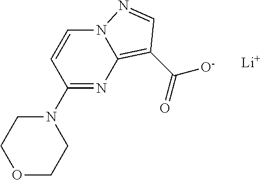 [SMILES: O=C([O-])c1cnn2ccc(N3CCOCC3)nc12 ; [Li+]]

A mixture of ethyl 5-(morpholin-4-yl)pyrazolo[1,5-a]pyrimidine-3-carboxylate (130 mg, 471 μmol), lithium hydroxide (1.2 ml, 1 M aqueous solution, 1.2 mmol), methanol (0.82 ml) and tetrahydrofuran (3.1 ml) were stirred at room temperature over night. For work-up, the reaction mixture was concentrated, diluted with water and acidified with hydrochloric acid. The precipitate was collected by filtration and dried under high vacuum at 50° C. to give the title compound (130 mg).

LC-MS (Method 1): Rₜ=0.58 min; MS (ESIneg): m/z=247.1 [M−H]⁻

### Intermediate I45

### ethyl 5-(pyrrolidin-1-yl)pyrazolo[1,5-a]pyrimidine-3-carboxylate

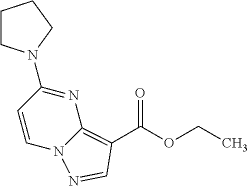 [SMILES: CCOC(=O)c1cnn2ccc(N3CCCC3)nc12]

Was prepared in analogy to the synthesis of ethyl 5-(4-methylpiperazin-1-yl)pyrazolo[1,5-a]pyrimidine-3-carboxylate using ethyl 5-chloropyrazolo[1,5-a]pyrimidine-3-carboxylate (677 mg, 3.00 mmol) and pyrrolidine (500 μl, 6.0 mmol) as starting materials to give the title compound (770 mg).

LC-MS (Method 1): Rₜ=0.93 min; MS (ESIpos): m/z=261.2 [M+H]⁺

1H-NMR (400 MHz, DMSO-d6) delta [ppm]: 1.258 (7.51), 1.276 (16.00), 1.294 (7.81), 1.339 (0.60), 1.930 (1.78), 1.944 (1.99), 1.990 (1.99), 2.005 (1.78), 2.074 (0.51), 2.523 (1.08), 3.471 (1.15), 3.490 (2.03), 3.507 (1.45), 3.583 (1.49), 3.601 (2.04), 3.618 (1.15), 4.153 (2.44), 4.170 (7.50), 4.188 (7.38), 4.206 (2.31), 6.494 (4.51), 6.513 (4.59), 8.168 (9.15), 8.661 (5.06), 8.680 (4.87).

### Intermediate I46

### lithium 5-(pyrrolidin-1-yl)pyrazolo[1,5-a]pyrimidine-3-carboxylate

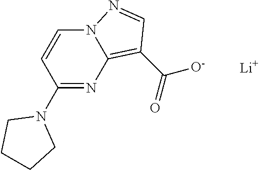 [SMILES: O=C([O-])c1cnn2ccc(N3CCCC3)nc12 ; [Li+]]

Was prepared in analogy to the synthesis of lithium 5-(morpholin-4-yl)pyrazolo[1,5-a]pyrimidine-3-carboxylat using ethyl 5-(pyrrolidin-1-yl)pyrazolo[1,5-a]pyrimidine-3-carboxylate (146 mg, 561 μmol) as starting material. For work-up, the reaction mixture was concentrated and the residue was azeotroped twice with toluene to give the title compound (175 mg) as crude product, which was used in the next step without further purification.

### Intermediate I47

### methyl 4-({[5-(methylcarbamoyl)-1H-imidazol-4-yl]carbonyl}amino)bicyclo[2.2.2]octane-1-carboxylate

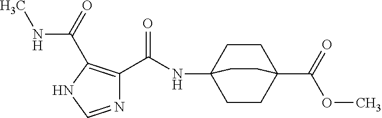

Methyl 4-aminobicyclo[2.2.2]octane-1-carboxylate (220 mg, 1.20 mmol, Cas No 135908-33-7) and N-ethyl-N-isopropylpropan-2-amine (0.31 ml, 1.8 mmol) were added to a suspension of 5,10-dioxo-5H,10H-diimidazo[1,5-a:1′,5′-d]pyrazine-1,6-dicarbonyl dichloride (188 mg, 0.600 mmol) in tetrahydrofuran (8.0 ml) and the mixture was stirred at room temperature. After 6 h, a solution of methanamine (0.60 ml, 1.2 mmol, 2M solution in tetrahydrofuran) and N-ethyl-N-isopropylpropan-2-amine (0.31 ml, 1.8 mmol) in tetrahydrofuran (8 ml) was added and the mixture was stirred over night at room temperature. For work-up, precipitates were filtered off and washed with tetrahydrofuran. The combined filtrates were concentrated and purified by flash chromatography (Snap Cartridge, hexanes/ethyl acetate gradient 50%->100% ethyl acetate) to provide the title compound (198 mg).

LC-MS (Method 2): Rₜ=0.89 min; MS (ESIpos) m/z=335.2 [M+H]⁺.

¹H-NMR (400 MHz, DMSO-d₆): δ [ppm]=13.32-12.85 (m, 1H), 11.42-10.51 (m, 1H), 8.93-8.16 (m, 1H), 7.74 (s, 1H), 3.57 (s, 3H), 2.78 (d, 3H), 2.06-1.90 (m, 6H), 1.89-1.70 (m, 6H).

### Intermediate I48

### 4-({[5-(methylcarbamoyl)-1H-imidazol-4-yl]carbonyl}amino)bicyclo[2.2.2]octane-1-carboxylic acid

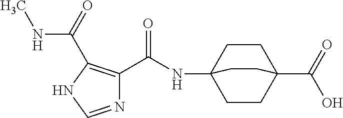 [SMILES: CNC(=O)c1[nH]cnc1C(=O)NC12CCC(C(=O)O)(CC1)CC2]

A mixture of methyl 4-({[5-(methylcarbamoyl)-1H-imidazol-4-yl]carbonyl}amino)bicyclo[2.2.2]octane-1-carboxylate (350 mg, 1.05 mmol) and lithium hydroxide (2.1 ml, 1.0 M aqueous solution, 2.1 mmol) in tetrahydrofuran (1.6 ml) and methanol (6.4 ml) was stirred over night at room temperature. For work-up, the reaction mixture was concentrated, water was added and the mixture was acidified with aqueous hydrochloric acid. The precipitate was collected by filtration, washed with water and ethanol and dried under high vacuum to yield the title compound (167 mg).

LC-MS (Method 1): Rₜ=0.71 min; MS (ESIpos) m/z=3212 [M+H]⁺.

¹H-NMR (400 MHz, DMSO-d6) delta [ppm]: 1.780 (8.94), 1.795 (13.23), 1.797 (14.19), 1.810 (12.90), 1.821 (16.00), 1.865 (0.67), 1.904 (2.15), 1.920 (12.23), 1.931 (11.45), 1.945 (12.61), 1.961 (7.83), 2.317 (0.52), 2.322 (1.16), 2.326 (1.58), 2.331 (1.11), 2.336 (0.52), 2.465 (0.57), 2.522 (3.83), 2.659 (0.54), 2.664 (1.21), 2.668 (1.63), 2.673 (1.16), 2.678 (0.54), 2.769 (8.06), 2.782 (8.74), 2.831 (1.16), 7.760 (15.02), 8.551 (1.73), 8.564 (1.76), 11.070 (3.57), 12.072 (0.93), 13.041 (2.02).

### Intermediate I49

### 4-[(pyrazolo[1,5-a]pyrimidin-3-ylcarbonyl)amino]bicyclo[2.2.2]octane-1-carboxylic acid

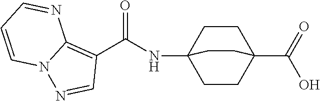 [SMILES: O=C(NC12CCC(C(=O)O)(CC1)CC2)c1cnn2cccnc12]

### Step 1:

(Benzotriazol-1-yloxy)tripyrrolidinophosphonium hexafluorophosphate (687 mg, 1.32 mmol) was added to a mixture of pyrazolo[1,5-a]pyrimidine-3-carboxylic acid (196 mg, 1.20 mmol, CAS No. 25940-35-6), methyl 4-aminobicyclo[2.2.2]octane-1-carboxylate (242 mg, 1.32 mmol, Cas No 135908-33-7) and N-ethyl-N-isopropylpropan-2-amine (0.84 ml, 4.80 mmol) in N,N-dimethylformamide (13 ml) and the mixture was stirred over night at room temperature.

For work-up the reaction mixture was concentrated under reduced pressure and the residue was purified by flash chromatogryph (Snap Cartridge, hexanes/ethyl acetate gradient 0%->100% ethyl acetate) to yield methyl 4-[(pyrazolo[1,5-a]pyrimidin-3-ylcarbonyl)amino]bicyclo[2.2.2]octane-1-carboxylate (453 mg, 115% yield).

LC-MS (Method 2): Rₜ=0.97 min; MS (ESIpos) m/z=329.2 [M+H]⁺.

¹H-NMR (400 MHz, DMSO-d₆): δ [ppm]=9.31 (dd, 1H), 8.79 (dd, 1H), 8.52 (s, 1H), 7.69 (s, 1H), 7.26 (dd, 1H), 3.59 (s, 3H), 2.08-1.94 (m, 6H), 1.92-1.77 (m, 6H).

### Step 2:

A mixture of methyl 4-[(pyrazolo[1,5-a]pyrimidin-3-ylcarbonyl)amino]bicyclo[2.2.2]octane-1-carboxylate (275 mg, 0.837 mmol) was stirred in a mixture of lithium hydroxide (2.1 ml, 2.1 mmol, 1 M aqueous solution), tetrahydrofuran (5.5 ml) and methanol (1.5 ml) over night at room temperature. For work-up, the reaction mixture was concentrated under reduced pressure, diluted with water and acidified with concentrated hydrochloric acid under cooling. The precipitated product was collected by filtration washed with water and ethanol and dried at 50° C. under high vacuum to yield the title (171 mg, 65% yield).

¹H-NMR (400 MHz, DMSO-d₆): δ [ppm]=12.10 (br. s, 1H), 9.31 (dd, 1H), 8.79 (dd, 1H), 8.52 (s, 1H), 7.68 (s, 1H), 7.26 (dd, 1H), 2.04-1.93 (m, 6H), 1.87-1.75 (m, 6H).

### Intermediate I50

### 4-[(imidazo[1,2-b]pyridazin-3-ylcarbonyl)amino]bicyclo[2.2.2]octane-1-carboxylic acid

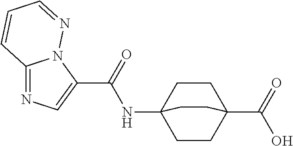 [SMILES: O=C(NC12CCC(C(=O)O)(CC1)CC2)c1cnc2cccnn12]

Was prepared in 2 steps from imidazo[1,2-b]pyridazine-3-carboxylic acid (196 mg, 1.20 mmol) and methyl 4-aminobicyclo[2.2.2]octane-1-carboxylate (242 mg, 1.32 mmol, Cas No 135908-33-7) in analogy to the synthesis of 4-[(pyrazolo[1,5-a]pyrimidin-3-ylcarbonyl)amino]bicyclo[2.2.2]octane-1-carboxylic acid to give the title compound (284 mg).

LC-MS (Method 1): Rₜ=0.73 min; MS (ESIpos) m/z=315.2 [M+H]⁺.

¹H-NMR (400 MHz, DMSO-d₆): δ [ppm]=12.14 (br. s., 1H), 8.78 (dd, 1H), 8.37-8.33 (m, 2H), 8.26 (s, 1H), 7.48-7.43 (m, 1H), 2.07-1.97 (m, 6H), 1.89-1.79 (m, 6H).

### Intermediate I51

### 4-{[(5,7-dimethylpyrazolo[1,5-a]pyrimidin-3-yl)carbonyl]amino}bicyclo[2.2.2]octane-1-carboxylic acid

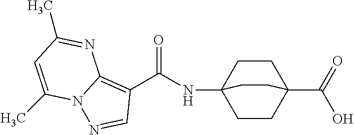 [SMILES: Cc1cc(C)n2ncc(C(=O)NC34CCC(C(=O)O)(CC3)CC4)c2n1]

Was prepared in 2 steps from 5,7-dimethylpyrazolo[1,5-a]pyrimidine-3-carboxylic acid (268 mg, 1.40 mmol) and methyl 4-aminobicyclo[2.2.2]octane-1-carboxylate (282 mg, 1.54 mmol, Cas No 135908-33-7) in analogy to the synthesis of 4-[(pyrazolo[1,5-a]pyrimidin-3-ylcarbonyl)amino]bicyclo[2.2.2]octane-1-carboxylic acid to give the title compound (300 mg).

LC-MS (Method 1): Rₜ=0.92 min; MS (ESIpos) m/z=343.2 [M+H]⁺.

¹H-NMR (400 MHz, DMSO-d₆): δ [ppm]=12.14 (br. s., 1H), 8.43 (s, 1H), 7.93 (s, 1H), 7.11 (d, 1H), 2.73 (d, 3H), 2.60 (s, 3H), 2.03-1.93 (m, 6H), 1.87-1.78 (m, 6H).

### Intermediate I52

### phenyl 5-({trans-4-[(2-chloro-5-fluorophenyl)carbamoyl]cyclohexyl}carbamoyl)-1H-imidazole-4-carboxylate

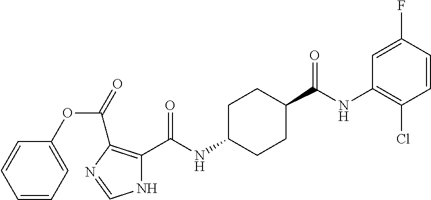 [SMILES: O=C(Oc1ccccc1)c1nc[nH]c1C(=O)N[C@H]1CC[C@H](C(=O)Nc2cc(F)ccc2Cl)CC1]

1-Chloro-N,N,2-trimethylprop-1-en-1-amine (748 mg, 5.60 mmol) was added to a solution of trans-4-({[4-(phenoxycarbonyl)-1H-imidazol-5-yl]carbonyl}amino)cyclohexanecarboxylic acid (1.00 g, 2.80 mmol) in dichloromethane (63 ml) and the mixture was stirred at room temperature for 30 min. Pyridine (0.68 ml, 8.4 mmol) and 2-chloro-5-fluoroaniline (448 mg, 3.08 mmol, CAS No. 452-83-5) were added and the mixture was stirred over night at room temperature. For work-up, water was added, the mixture was extracted with ethyl acetate and the organic phase was washed with a saturated sodium bicarbonate solution and water. The organic phase was dried by filtration through a silicone filter and the filtrate was concentrated. The residue was stirred with methanol, the precipitate formed was collected by filtration, washed with methanol and dried give the title compound (480 mg).

LC-MS (Method 2): Rₜ=1.00 min; MS (ESIpos) m/z=485.3 [M+H]⁺.

¹H-NMR (400 MHz, DMSO-d₆): δ [ppm]=13.66-13.46 (m, 1H), 9.49 (s, 1H), 9.46-9.19 (m, 1H), 7.95 (s, 1H), 7.67 (dd, 1H), 7.55-7.45 (m, 3H), 7.37-7.25 (m, 3H), 7.09-7.01 (m, 1H), 3.82-3.64 (m, 1H), 2.06-1.84 (m, 4H), 1.60-1.45 (m, 2H), 1.36-1.19 (m, 2H).

### Intermediate I53

### trans-4-[(pyrazolo[1,5-a]pyrimidin-3-ylcarbonyl)amino]cyclohexanecarboxylic acid

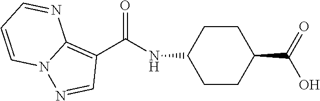

### Step 1:

1-Chlor-1-dimethylamino-2-methyl-1-propen (1.31 g, 9.81 mmol) was added to a solution of pyrazolo[1,5-a]pyrimidine-3-carboxylic acid (800 mg, 4.90 mmol, CAS No. 25940-35-6) in dichloromethane (15 ml) and the mixture was stirred at room temperature for 30 min. Pyridine (1.19 ml, 14.7 mmol) and methyl trans-4-aminocyclohexanecarboxylate hydrochloride (950 mg, 0.418 mmol, Cas No. 61367-C₇-5) were added and the mixture was stirred for 3 days at room temperature. For work-up water was added and the mixture was extracted with dichloromethane. The organic phase was washed with water, filtrated through a silicone filter and concentrated. The crude product purified by flash chromatography (25 g Snap cartridge, hexanes/ethyl acetate gradient 40%->100% ethyl acetate) to yield methyl trans-4-[(pyrazolo[1,5-a]pyrimidin-3-ylcarbonyl)amino]cyclohexanecarboxylate (722 mg, 48% yield).

¹H-NMR (400 MHz, DMSO-d₆): δ [ppm]=9.32 (dd, 1H), 8.82 (dd, 1H), 8.57 (s, 1H), 7.78 (d, 1H), 7.27 (dd, 1H), 3.89-3.73 (m, 1H), 3.61 (s, 3H), 2.43-2.30 (m, 1H), 2.07-1.89 (m, 4H), 1.59-1.28 (m, 4H).

### Step 2:

A mixture of methyl trans-4-[(pyrazolo[1,5-a]pyrimidin-3-ylcarbonyl)amino]cyclohexanecarboxylate (711 mg, 2.39 mmol) was stirred in a mixture of lithium hydroxide (11.9 ml, 11.9 mmol, 1 M aqueous solution), tetrahydrofuran (15.6 ml) and methanol (4.2 ml) for 3 days at room temperature. For work-up, the organic solvents were removed under reduced pressure, the mixture was diluted with water and acidified with concentrated hydrochloric acid under cooling. The precipitated product was collected by filtration and dried at 50° C. under high vacuum to yield the title (586 mg, 83% yield).

LC-MS (Method 1): Rₜ=0.67 min; MS (ESIpos) m/z=289.1 [M+H]⁺.

¹H-NMR (400 MHz, DMSO-d₆): δ [ppm]=12.33-11.85 (m, 1H), 9.32 (dd, 1H), 8.82 (dd, 1H), 8.57 (s, 1H), 7.78 (d, 1H), 7.27 (dd, 1H), 3.86-3.73 (m, 1H), 2.24 (s, 1H), 2.07-1.90 (m, 4H), 1.54-1.27 (m, 4H).

### Intermediate I54

### trans-4-[(imidazo[1,2-b]pyridazin-3-ylcarbonyl)amino]cyclohexanecarboxylic acid

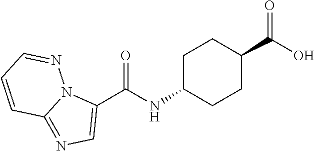 [SMILES: O=C(N[C@H]1CC[C@H](C(=O)O)CC1)c1cnc2cccnn12]

### Step 1:

1-Chlor-1-dimethylamino-2-methyl-1-propen (357 m g, 3.67 mmol) was added to a solution of imidazo[1,2-b]pyridazine-3-carboxylic acid (218 mg, 1.34 mmol, Cas No 1308384-58-8) in dichloromethane (4.2 ml) and the mixture was stirred at room temperature for 30 min. Pyridine (0.32 ml, 4.0 mmol) and methyl trans-4-aminocyclohexanecarboxylate hydrochloride (259 mg, 1.34 mmol, Cas No. 61367-C₇-5) were added and the mixture was stirred over night at room temperature. For work-up water was added and the mixture was extracted with dichloromethane. The organic phase was washed with water, filtrated through a silicone filter and concentrated to give methyl trans-4-[(imidazo[1,2-b]pyridazin-3-ylcarbonyl)amino]cyclohexanecarboxylate (350 mg, 86% yield). The crude product was used in the next step without further purification.

LC-MS (Method 2): Rₜ=0.84 min; MS (ESIpos) m/z=303.2 [M+H]⁺.

¹H-NMR (400 MHz, DMSO-d₆): δ [ppm]=8.78 (dd, 1H), 8.44 (d, 1H), 8.35 (dd, 1H), 8.30 (s, 1H), 7.46 (dd, 1H), 3.93-3.76 (m, 1H), 3.62 (s, 3H), 2.42-2.30 (m, 1H), 2.06-1.94 (m, 4H), 1.57-1.33 (m, 4H).

### Step 2:

A mixture of methyl trans-4-[(imidazo[1,2-b]pyridazin-3-ylcarbonyl)amino]cyclohexanecarboxylate (350 mg, 1.16 mmol) was stirred in a mixture of lithium hydroxide (5.8 ml, 5.8 mmol, 1 M aqueous solution), tetrahydrofuran (7.6 ml) and methanol (2.0 ml) over night at room temperature. For work-up, the organic solvents were removed under reduced pressure, the mixture was diluted with water and acidified with concentrated hydrochloric acid under cooling. The precipitated product was collected by filtration and dried at 50° C. under high vacuum to yield the title. (57 mg, 17% yield).

LC-MS (Method 1): Rₜ=0.62 min; MS (ESIpos) m/z=289.2 [M+H]⁺.

¹H-NMR (400 MHz, DMSO-d₆): δ [ppm]=12.89-11.56 (m, 1H), 8.81-8.76 (m, 1H), 8.45 (d, 1H), 8.37-8.33 (m, 1H), 8.30 (s, 1H), 7.50-7.45 (m, 1H), 3.91-3.78 (m, 1H), 2.29-2.18 (m, 1H), 2.06-1.93 (m, 4H), 1.57-1.33 (m, 4H).

### Intermediate I55

### trans-4-{[(5,7-dimethylpyrazolo[1,5-a]pyrimidin-3-yl)carbonyl]amino}cyclohexanecarboxylic acid

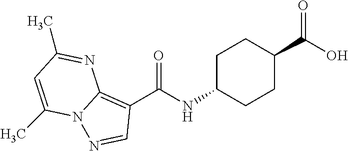 [SMILES: Cc1cc(C)n2ncc(C(=O)N[C@H]3CC[C@H](C(=O)O)CC3)c2n1]

### Step 1:

(Benzotriazol-1-yloxy)tripyrrolidinophosphonium hexafluorophosphate (980 mg, 1.88 mmol) was added to a mixture of 5,7-dimethylpyrazolo[1,5-a]pyrimidine-3-carboxylic acid (300 mg, 1.57 mmol, CAS no 90349-23-8), trans-4-aminocyclohexanecarboxylate hydrochloride (274 mg, 1.41 mmol, Cas No. 61367-07-5) and N-ethyl-N-isopropylpropan-2-amine (1.1 ml, 6.3 mmol) in N,N-dimethylformamide (9.8 ml) and the mixture was stirred for 3 h at room temperature. For work-up the reaction mixture was concentrated under reduced pressure. The residue was dissolved with dichloromethane and the organic phase was washed with water, filtrated through a silicone filter and concentrated. The crude product crystalized upon standing to give methyl trans-4-{[(5,7-dimethylpyrazolo[1,5-a]pyrimidin-3-yl)carbonyl]amino}cyclohexanecarboxylate (237 mg, 45% yield).

LC-MS (Method 1): Rₜ=1.01 min; MS (ESIneg) m/z=329.2 [M−H]⁻.

¹H-NMR (400 MHz, DMSO-d₆): δ [ppm]=8.49 (s, 1H), 7.97 (d, 1H), 7.12 (d, 1H), 3.86-3.70 (m, 1H), 3.61 (s, 3H), 2.74 (d, 3H), 2.62 (s, 3H), 2.43-2.31 (m, 1H), 2.08-1.90 (m, 4H), 1.58-1.29 (m, 4H).

### Step 2:

A mixture of methyl trans-4-{[(5,7-dimethylpyrazolo[1,5-a]pyrimidin-3-yl)carbonyl]amino}cyclohexanecarboxylate (235 mg, 0.711 mmol) was stirred in a mixture of lithium hydroxide (3.6 ml, 3.6 mmol, 1 M aqueous solution), tetrahydrofuran (4.6 ml) and methanol (1.2 ml) over night at room temperature. For work-up, the organic solvents were removed under reduced pressure, the mixture was diluted with water and acidified with concentrated hydrochloric acid under cooling. The precipitated product was collected by filtration and dried to yield the title. (174 mg, 77% yield).

LC-MS (Method 2): Rₜ=0.46 min; MS (ESIpos) m/z=317.2 [M+H]⁺.

¹H-NMR (400 MHz, DMSO-d₆): δ [ppm]=12.97-10.65 (m, 1H), 8.49 (s, 1H), 7.98 (d, 1H), 7.12 (d, 1H), 3.86-3.69 (m, 1H), 2.74 (d, 3H), 2.62 (s, 3H), 2.32-2.21 (m, 1H), 2.07-1.87 (m, 4H), 1.54-1.24 (m, 4H).

### Intermediate I56

### ethyl 4-{[tert-butyl(dimethyl)silyl]oxy}cyclohexanecarboxylate (mixture of cis-/trans-isomers)

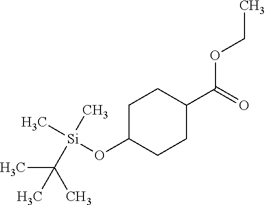 [SMILES: CCOC(=O)C1CCC(O[Si](C)(C)C(C)(C)C)CC1]

tert-Butyldimethylsilyl chloride (26.3 g, 174 mmol) was added to a solution of ethyl 4-hydroxycyclohexanecarboxylate (25.0 g, 145 mmol, mixture of cis-/trans-isomers, Cas No 17159-80-7) and imidazole (24.7 g, 363 mmol) in N,N-dimethylformamide (36 ml) and the mixture was stirred over night at room temperature. For work-up, water was added and the mixture was extracted with tert-butyl methyl ether (3×). The combined organic phases were washed with brine, filtrated through a silicone filter and concentrated under reduced pressure to yield ethyl 4-{[tert-butyl(dimethyl)silyl]oxy}cyclohexanecarboxylate (43.1 g, 104% yield) which was used in the next step without further purification.

¹H-NMR (400 MHz, DMSO-d₆, mixture of isomers): δ [ppm]=4.10-3.99 (m, 2H), 3.93-3.86 (m, 0.7H), 3.63-3.51 (m, 0.3H), 2.39-2.28 (m, 0.8H), 2.27-2.14 (m, 0.3H), 1.91-1.21 (m, 8H), 1.20-1.13 (m, 3H), 0.89-0.79 (m, 9H), 0.08-0.00 (m, 6H).

### Intermediate I57

### ethyl 4-{[tert-butyl(dimethyl)silyl]oxy}-1-(2-chloroethyl)cyclohexanecarboxylate (mixture of cis-/trans-isomers)

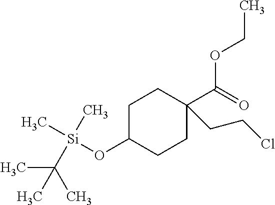 [SMILES: CCOC(=O)C1(CCCl)CCC(O[Si](C)(C)C(C)(C)C)CC1]

Lithium diisopropylamide (20.9 ml, 41.9 mmol, 1.8 M solution in tetrahydrofuran) was added dropwise to a solution of ethyl 4-{[tert-butyl(dimethyl)silyl]oxy}cyclohexanecarboxylate (10.0 g, 34.9 mmol) in tetrahydrofuran (47 ml) at −78° C. and the mixture was stirred for 30 min at that temperature. 1-Bromo-2-chloroethane (4.3 ml, 52 mmol) was added and the mixture was stirred for 1 h at −78° C. and then warmed during 2 h to room temperature. For work-up, water was added and the mixture was extracted with tert-butyl methyl ether (3×). The combined organic phases were washed with brine and filtrated through a silicone filter and concentrated under reduced pressure. The crude product (12 g) was purified by flash chromatogryph (340 g Snap cartridge, hexanes/ethyl acetate gradient) to give ethyl 4-{[tert-butyl(dimethyl)silyl]oxy}-1-(2-chloroethyl)cyclohexanecarboxylate (9.1 g, 75% yield as mixture of isomers, ratio 5:1 by GC-MS).

GC-MS (Method 10): Rₜ=20.27 min (minor isomer); 20.50 min (major isomer); MS (Cl) m/z=366.2 [M+NH4]⁺ and 349.2 [M+H]⁺.

### Intermediate I58

### 8-{[tert-butyl(dimethyl)silyl]oxy}-2-(2-chloro-4-fluorophenyl)-2-azaspiro[4.5]decan-1-one

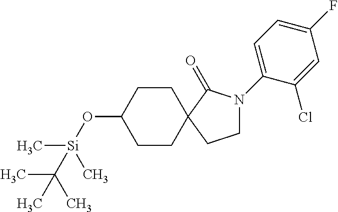 [SMILES: CC(C)(C)[Si](C)(C)OC1CCC2(CC1)CCN(c1ccc(F)cc1Cl)C2=O]

Lithium hexamethyldisilazide (22.9 ml, 22.9 mmol, 1 M solution in tetrahydrofuran) was added over 5 min to solution of 2-chloro-4-fluoroaniline (1.84 g, 12.6 mmol, Cas No 2106-02-7) in tetrahydrofuran (60 ml) at −78° C. and the mixture was stirred at −78° C. for 1 h. A solution of ethyl 4-{[tert-butyl(dimethyl)silyl]oxy}-1-(2-chloroethyl)cyclohexanecarboxylate (mixture of cis-/trans-isomers) (4.00 g, 11.5 mmol) in tetrahydrofuran (60 ml) was added and the mixture was stirred for 2 h at −78° C. and the for 3 days at room temperature. For work-up, the reaction mixture was poured into a mixture of water and saturated sodium bicarbonate solution, extracted with ethyl acetate (3×) and the combined organic phases were washed with brine, filtrated through a silicone filter and concentrated under reduced pressured. The residue was purified by flash chromatography (Snap Cartdidge, hexanes/ethyl acetate gradient) to give 8-{[tert-butyl(dimethyl)silyl]oxy}-2-(2-chloro-4-fluorophenyl)-2-azaspiro[4.5]decan-1-one in 2 fractions. Fraction 1 (1.88 g, isomer 1, contains ca 20 mol % 2-chloro-4-fluoroaniline) and fraction 2 (485 mg, isomer 2).

### Fraction 1 (Isomer 1):

LC-MS (Method 1): Rₜ=1.68 min; MS (ESIpos) m/z=412.2 [M+H]⁺.

¹H-NMR (400 MHz, DMSO-d₆): δ [ppm]=7.58 (dd, 1H), 7.46 (dd, 1H), 7.30 (td, 1H), 3.97-3.92 (m, 1H), 3.59 (t, 2H), 2.06 (t, 2H), 2.01-1.92 (m, 2H), 1.71-1.54 (m, 4H), 1.36-1.29 (m, 2H), 0.92-0.86 (m, 9H), 0.08-0.03 (m, 6H).

### Fraction 2 (Isomer 2):

LC-MS (Method 1): Rₜ=1.67 min; MS (ESIpos) m/z=412.2 [M+H]⁺.

¹H-NMR (400 MHz, DMSO-d₆): δ [ppm]=7.57 (dd, 1H), 7.45 (dd, 1H), 7.33-7.25 (m, 1H), 3.70-3.52 (m, 3H), 2.07 (t, 2H), 1.84-1.74 (m, 2H), 1.62-1.54 (m, 4H), 1.42-1.28 (m, 2H), 0.89-0.83 (m, 9H), 0.07-0.01 (m, 6H)

### Intermediate I59

### 2-(2-chloro-4-fluorophenyl)-8-hydroxy-2-azaspiro[4.5]decan-1-one (isomer 1)

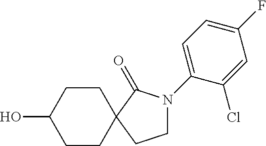 [SMILES: O=C1N(c2ccc(F)cc2Cl)CCC12CCC(O)CC2]

Tetra-N-butylammonium fluoride (9.1 ml, 9.1 mmol, 1 M solution in tetrahyrofuran) was added to a solution of 8-{[tert-butyl(dimethyl)silyl]oxy}-2-(2-chloro-4-fluorophenyl)-2-azaspiro[4.5]decan-1-one (isomer 1) (1.88 g, fraction 1) in tetrahydrofuran (43 ml) and the mixture was stirred at room temperature for 12 h. The mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (Snap cartridge, hexanes/ethyl acetate gradient, 20%->50% ethyl acetate) to provide 2-(2-chloro-4-fluorophenyl)-8-hydroxy-2-azaspiro[4.5]decan-1-one (isomer 1) (1.08 g).

LC-MS (Method 2): Rₜ=0.98 min; MS (ESIpos) m/z=298.1 [M+H]⁺.

¹H-NMR (400 MHz, DMSO-d₆): δ [ppm]=7.58 (dd, 1H), 7.46 (dd, 1H), 7.30 (td, 1H), 4.40 (d, 1H), 3.78-3.72 (m, 1H), 3.59 (t, 2H), 2.04 (t, 2H), 2.00-1.90 (m, 2H), 1.74-1.62 (m, 2H), 1.60-1.47 (m, 2H), 1.36-1.23 (m, 2H).

### Intermediate I60

### 2-(2-chloro-4-fluorophenyl)-8-hydroxy-2-azaspiro[4.5]decan-1-one (isomer 2)

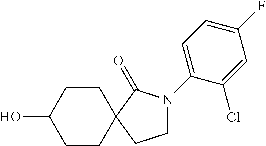 [SMILES: O=C1N(c2ccc(F)cc2Cl)CCC12CCC(O)CC2]

Was prepared in analogy to the synthesis of 2-(2-chloro-4-fluorophenyl)-8-hydroxy-2-azaspiro[4.5]decan-1-one (isomer 1) from of 8-{[tert-butyl(dimethyl)silyl]oxy}-2-(2-chloro-4-fluorophenyl)-2-azaspiro[4.5]decan-1-one (isomer 2) (480 mg, fraction 2) using tetra-N-butylammonium fluoride (2.3 ml, 2.3 mmol, 1 M solution in tetrahyrofuran) go give 2-(2-chloro-4-fluorophenyl)-8-hydroxy-2-azaspiro[4.5]decan-1-one (isomer 2) (300 mg).

LC-MS (Method 2): Rₜ=0.90 min; MS (ESIpos) m/z=298.1 [M+H]⁺.

¹H-NMR (400 MHz, DMSO-d₆): δ [ppm]=7.58 (dd, 1H), 7.46 (dd, 1H), 7.34-7.27 (m, 1H), 4.63 (d, 1H), 3.59 (t, 2H), 3.47-3.35 (m, 1H), 2.07 (t, 2H), 1.85-1.76 (m, 2H), 1.62-1.49 (m, 4H), 1.35-1.18 (m, 2H).

### Intermediate I61

### 2-[2-(2-chloro-4-fluorophenyl)-1-oxo-2-azaspiro[4.5]dec-8-yl]-1H-isoindole-1,3(2H)-dione (isomer 1)

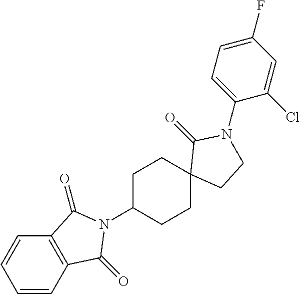 [SMILES: O=C1c2ccccc2C(=O)N1C1CCC2(CC1)CCN(c1ccc(F)cc1Cl)C2=O]

Diisopropyl azodicarboxylate (1.1 ml, 5.4 mmol) was added dropwise to a mixture of 2-(2-chloro-4-fluorophenyl)-8-hydroxy-2-azaspiro[4.5]decan-1-one (isomer 1) (1.08 g, 3.61 mmol), phthalimide (797 mg, 5.42 mmol) and triphenylphosphine (1.42 g, 5.42 mmol) in tetrahydrofuran (30 ml) and the mixture was stirred for 12 h at room temperature. For work-up, the reaction mixture was concentrated and the residue was purified by flash chromatography (hexanes/ethyl acetate gradient) to yield 2-[2-(2-chloro-4-fluorophenyl)-1-oxo-2-azaspiro[4.5]dec-8-yl]-1H-isoindole-1,3(2H)-dione (isomer 1) (697 mg).

LC-MS (Method 2): Rₜ=1.31 min; MS (ESIpos) m/z=427.2 [M+H]⁺.

¹H-NMR (400 MHz, DMSO-d₆): δ [ppm]=7.90-7.82 (m, 4H), 7.63-7.56 (m, 1H), 7.49 (dd, 1H), 7.32 (td, 1H), 4.12-3.98 (m, 1H), 3.66 (t, 2H), 2.35-2.17 (m, 4H), 1.80-1.58 (m, 6H).

### Intermediate I62

### 2-[2-(2-chloro-4-fluorophenyl)-1-oxo-2-azaspiro[4.5]dec-8-yl]-1H-isoindole-1,3(2H)-dione (isomer 2)

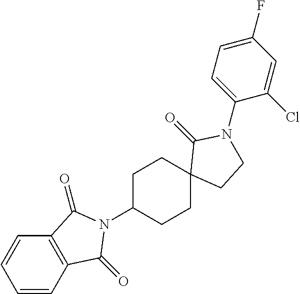 [SMILES: O=C1c2ccccc2C(=O)N1C1CCC2(CC1)CCN(c1ccc(F)cc1Cl)C2=O]

Was prepared in analogy to 2-[2-(2-chloro-4-fluorophenyl)-1-oxo-2-azaspiro[4.5]dec-8-yl]-1H-isoindole-1,3(2H)-dione (isomer 1) using 2-(2-chloro-4-fluorophenyl)-8-hydroxy-2-azaspiro[4.5]decan-1-one (isomer 2) (308 mg, 1.03 mmol) as starting material to give 2-[2-(2-chloro-4-fluorophenyl)-1-oxo-2-azaspiro[4.5]dec-8-yl]-1H-isoindole-1,3(2H)-dione (isomer 2) (190 mg).

LC-MS (Method 1): Rₜ=1.34 min; MS (ESIpos) m/z=427.2 [M+H]⁺.

¹H-NMR (400 MHz, DMSO-d₆): δ [ppm]=7.87-7.79 (m, 1H), 7.61 (dd, 1H), 7.52 (dd, 1H), 7.33 (td, 1H), 4.10-3.98 (m, 1H), 3.61 (t, 2H), 2.90-2.75 (m, 2H), 2.15-2.05 (m, 2H), 2.00 (t, 2H), 1.62-1.49 (m, 4H).

### Intermediate I63

### 8-amino-2-(2-chloro-4-fluorophenyl)-2-azaspiro[4.5]decan-1-one (isomer 1)

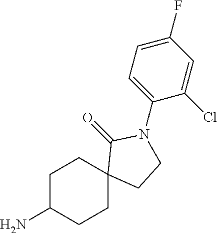 [SMILES: NC1CCC2(CC1)CCN(c1ccc(F)cc1Cl)C2=O]

A mixture of 2-[2-(2-chloro-4-fluorophenyl)-1-oxo-2-azaspiro[4.5]dec-8-yl]-1H-isoindole-1,3(2H)-dione (isomer 1) (585 mg, 1.37 mmol) and hydrazine hydrate (0.34 ml) in ethanol (12 ml) was stirred at 80° C. for 3 h. Upon cooling, the precipitate was filtrated off, washed with ethanol and the filtrate was concentrated to give the title compound (457 mg).

LC-MS (Method 2): Rₜ=0.94 min; MS (ESIpos) m/z=297.1 [M+H]⁺.

### Intermediate I64

### 8-amino-2-(2-chloro-4-fluorophenyl)-2-azaspiro[4.5]decan-1-one (isomer 2)

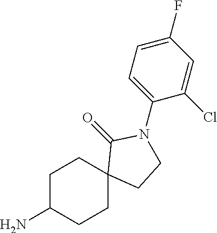 [SMILES: NC1CCC2(CC1)CCN(c1ccc(F)cc1Cl)C2=O]

A mixture of 2-[2-(2-chloro-4-fluorophenyl)-1-oxo-2-azaspiro[4.5]dec-8-yl]-1H-isoindole-1,3(2H)-dione (isomer 2) (188 mg, 0.440 mmol) and hydrazine hydrate (150 μl) in ethanol (4.0 ml) was stirred at 80° C. for 3 h. The reaction mixture was concentrated under reduced pressure and the crude product was codestilled with dichloromethane (2×) to give the title compound (200 mg) which was then used without further purification in the next step.

### Intermediate I65

### phenyl 5-{[2-(2-chloro-4-fluorophenyl)-1-oxo-2-azaspiro[4.5]dec-8-yl]carbamoyl}-1H-imidazole-4-carboxylate (isomer 1)

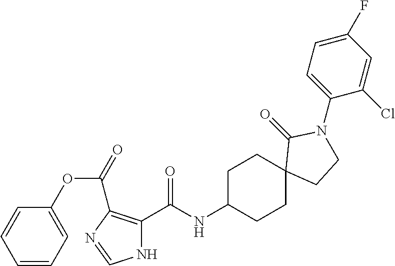 [SMILES: O=C(Oc1ccccc1)c1nc[nH]c1C(=O)NC1CCC2(CC1)CCN(c1ccc(F)cc1Cl)C2=O]

To a suspension of diphenyl 5,10-dioxo-5H,10H-diimidazo[1,5-a:1′,5′-d]pyrazine-1,6-dicarboxylate (1.67 g, 3.90 mmol) in 100 ml tetrahydrofuran were added 1.08 ml (7.80 mmol) trimethylamine and the mixture was heated to 60° C. 8-amino-2-(2-chloro-4-fluorophenyl)-2-azaspiro[4.5]decan-1-one (isomer 1) (1.93 g, 6.50 mmol) dissolved in 100 ml tetrahydrofuran was added over a period of 45 minutes and the mixture was stirred for 2.5 h at 60° C. The reaction mixture was concentrated under reduced pressure and the crude product was purified by flash chromatography (dichloromethane/tetrahydrofuran) to give the title compound as a solid material (3.30 g, isomer 1)

LC-MS (Method 1): Rₜ=1.18 min; MS (ESIpos) m/z=511.1 [M+H]⁺.

### Intermediate I66

### 8-amino-2-(2-chloro-4,5-difluorophenyl)-2-azaspiro[4.5]decan-1-one (isomer 1)

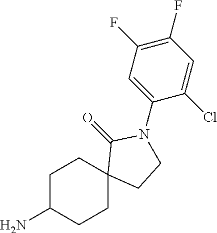 [SMILES: NC1CCC2(CC1)CCN(c1cc(F)c(F)cc1Cl)C2=O]

Was prepared in analogy to the synthesis of 8-amino-2-(2-chloro-4-fluorophenyl)-2-azaspiro[4.5]decan-1-one (isomer 1, intermediate I63 in 4 steps from ethyl 4-{[tert-butyl(dimethyl)silyl]oxy}-1-(2-chloroethyl)cyclohexanecarboxylate (mixture of cis-/trans-isomers) (2.00 g, 5.73 mmol) and 2-chloro-4,5-difluoroaniline (1.03 g, 6.30 mmol, CAS No 2613-32-3) as starting materials to give the title compound as crude product (326 mg) which was used in the next step without further purification.

LC-MS (Method 2): Rₜ=0.96 min; MS (ESIpos) m/z=315.1 [M+H]⁺.

### Intermediate I67

### methyl 4-amino-3-methylcyclohexanecarboxylate hydrochloric acid salt (mixture of isomers)

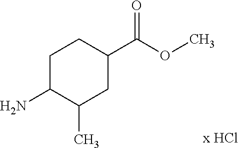

An autoclave was charged with a mixture of methyl 4-amino-3-methylbenzoate (10.0 g, 60.5 mmol, Cas No 18595-14-7), rhodium 5% on charcoal (3.74 g, 50% wet), acetic acid (5.2 ml) and ethanol (100 ml) and pressurized with hydrogen (12 bar). The mixture was stirred at 80 C for 22 h. For work-up, the catalyst was filtrate d off, washed with ethanol and the filtrate was concentrated under reduced pressure. The residue was dissolved in dichloromethane (250 ml) and hydrochloric acid (30 ml, 120 mmol, 4 M in dioxane) was added. The mixture was concentrated unreduced pressure and the residue was codestilled with toluene (2×) to give the crude product as a mixture of isomers which was used without further purification.

### Intermediate I68

### methyl 3-methyl-4-({[5-(methylcarbamoyl)-1H-imidazol-4-yl]carbonyl}amino)cyclohexanecarboxylate (mixture of isomers)

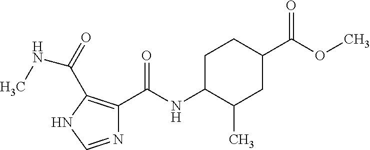

A solution of methyl 4-amino-3-methylcyclohexanecarboxylate hydrochloric acid salt (mixture of isomers) (1.87 g, 9.00 mmol) and N-ethyl-N-isopropylpropan-2-amine (3.9 ml, 23 mmol) in tetrahydrofuran (50 ml) was added to a suspension of 5,10-dioxo-5H,10H-diimidazo[1,5-a:1′,5′-d]pyrazine-1,6-dicarbonyl dichloride (1.41 g, 4.5 mmol) in tetrahydrofuran (100 ml) and the mixture was stirred at room temperature. After 6 h, methanamine (4.5 ml, 9.0 mmol, 2 M solution in tetrahydrofuran) and N-ethyl-N-isopropylpropan-2-amine (2.4 ml, 14 mmol) were added and the mixture was stirred over night at room temperature. For work-up, the reaction mixture was concentrated under reduced pressure and the residue was purified by flash chromatography (Snap Cartridge, hexanes/ethyl acetate gradient) to provide the title compound as mixture of isomers (860 mg).

LC-MS (Method 2): Rₜ=0.84 min; MS (ESIpos) m/z=232.2 [M+H]⁺.

¹H-NMR (400 MHz, DMSO-d₆, characteristic signals for all isomers given): δ [ppm]=13.28-13.01 (m, 1H), 11.32-10.73 (m, 1H), 8.65-8.45 (m, 1H), 7.78 (s, 1H), 3.67-3.55 (m, 3H), 2.86-2.73 (m, 3H), 0.95-0.79 (m, 3H).

### Intermediate I69

### methyl 4-{[(5,7-dimethylpyrazolo[1,5-a]pyrimidin-3-yl)carbonyl]amino}-3-methylcyclohexanecarboxylate (mixture of isomers)

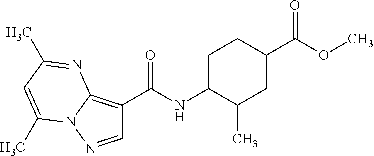

(Benzotriazol-1-yloxy)tripyrrolidinophosphonium hexafluorophosphate (3.33 g, 6.40 mmol) was added to a mixture of 5,7-dimethylpyrazolo[1,5-a]pyrimidine-3-carboxylic acid (1.11 g, 5.82 mmol), methyl 4-amino-3-methylcyclohexanecarboxylate hydrochloric acid salt (mixture of isomers) (1.33 g, 6.40 mmol) and N-ethyl-N-isopropylpropan-2-amine (5.1 ml, 29 mmol) in N,N-dimethylformamide (67 ml) and the mixture was stirred over night at room temperature. (Benzotriazol-1-yloxy)tripyrrolidinophosphonium hexafluorophosphate (1.97 g, 3.78 mmol) and N-ethyl-N-isopropylpropan-2-amine (2.5 ml, 15 mmol) were added and the mixture was stirred for 24 h at room temperature. For work-up, water was added and the mixture was extracted with ethyl acetate (3×). The combined organic phases were washed with brine, filtrated through a silicone filter and concentrated. The aqueous phase was extracted with a mixture of dichloromethane and methanol (4:1) and the combined organic phases were filtrated through a silicon filter and concentrated. The combined residues were purified by flash chromatography (100 g Snap Cartridge, hexanes/ethyl acetate gradient, 20%->100% ethyl acetate) to give the title compound as mixture of isomers (280 mg) which was used in the next step without purification.

### Intermediate I70

### 4-{[(5,7-dimethylpyrazolo[1,5-a]pyrimidin-3-yl)carbonyl]amino}-3-methylcyclohexanecarboxylic acid (mixture of isomers)

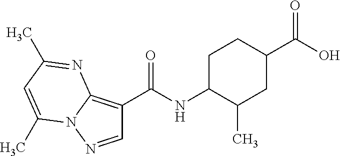 [SMILES: Cc1cc(C)n2ncc(C(=O)NC3CCC(C(=O)O)CC3C)c2n1]

A mixture of methyl 4-{[(5,7-dimethylpyrazolo[1,5-a]pyrimidin-3-yl)carbonyl]amino}-3-methylcyclohexanecarboxylate (280 mg, mixture of isomers) and sodium hydroxide (2.0 ml, 2.0 M aqueous solution, 4.1 mmol) in tetrahydrofuran (4.0 ml) and methanol (1.0 ml) was stirred at room temperature over night. For work-up, water (3 ml) was added and the organic solvents were removed under reduced pressure. The mixture was acidified with hydrochloric acid (2N) and then extracted with a mixture of dichloromethane and methanol (4:1) (3×). The combined organic phases were washed with brine, filtrated through a phase separator and concentrated to give the title compound as mixture of isomers (0.23 g) which was used in the next step without further purification.

### Intermediate I71

### phenyl 5-[(trans-4-{[2-chloro-5-(trifluoromethyl)phenyl]carbamoyl}cyclohexyl)carbamoyl]-1H-imidazole-4-carboxylate

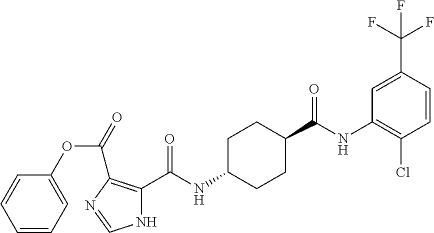 [SMILES: O=C(Oc1ccccc1)c1nc[nH]c1C(=O)N[C@H]1CC[C@H](C(=O)Nc2cc(C(F)(F)F)ccc2Cl)CC1]

1-Chloro-N,N,2-trimethylprop-1-en-1-amine (780 mg, 5.91 mmol) was added to a solution of trans-4-({[4-(phenoxycarbonyl)-1H-imidazol-5-yl]carbonyl}amino)cyclohexanecarboxylic acid (1.6 g, 2.96 mmol) in dichloromethane (66 ml) and the mixture was stirred at room temperature for 30 min. Pyridine (0.72 ml, 8.9 mmol) and 2-chloro-5-(trifluoromethyl)aniline (636 mg, 3.25 mmol, CAS No. 121-50-6,) were added and the mixture was stirred over night at room temperature. For work-up, water was added, the mixture was extracted with dichloromethane and the organic phase was washed with brine, dried by filtration through a silicone filter and the filtrate was concentrated. The residue was stirred with a mixture of dichloromethane and methanol. The precipitate formed was collected by filtration, washed with methanol and dried give the title compound (550 mg, 33% yield).

LC-MS (Method 2): Rₜ=1.15 min; MS (ESIneg) m/z=533.3 [M−H]⁻.

¹H-NMR (400 MHz, DMSO-d₆): δ [ppm]=13.57 (br. s, 1H), 9.68 (s, 1H), 9.50-9.18 (m, 1H), 8.13 (d, 1H), 7.95 (s, 1H), 7.77-7.72 (m, 1H), 7.57-7.45 (m, 3H), 7.37-7.26 (m, 3H), 3.81-3.67 (m, 1H), 2.06-1.83 (m, 4H), 1.62-1.44 (m, 2H), 1.38-1.17 (m, 2H).

### Intermediate I72

### 2-[(2-chloro-4-fluorophenyl)amino]ethanol

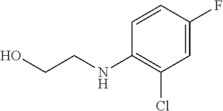 [SMILES: OCCNc1ccc(F)cc1Cl]

To a mixture of 1.47 g (10.0 mmol) 2-chloro-4-fluoroaniline and 1.25 g (10.0 mmol) of 2-bromoethanol in 10 ml toluene were added 1.74 (10.0 mmol)N-ethyl-N-isopropylpropan-2-amine and the mixture was stirred for 90 minutes at 100° C. The mixture was heated in to 150° C. in a microwave for 2 hours. The resulting precipitate was filters of and the filtrate was purified by flash column chromatography to give 1.05 g of the title compound as a solid material.

LC-MS (Method 2): Rₜ=0.96 min; MS (ESIpos) m/z=190.0 [M+H]⁺.

¹H-NMR (400 MHz, DMSO-d₆), δ [ppm]=7.26 (dd, 1H), 7.04 (td, 1H), 6.74 (dd, 1H), 5.04 (t, 1H), 4.84 (t, 1H), 3.59 (q, 2H), 3.16 (q, 2H).

### Intermediate I73

### phenyl 5-({trans-4-[(2-chloro-4-fluorophenyl)(2-hydroxyethyl)carbamoyl]cyclohexyl}carbamoyl)-1H-imidazole-4-carboxylate

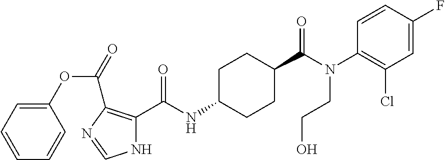 [SMILES: O=C(Oc1ccccc1)c1nc[nH]c1C(=O)N[C@H]1CC[C@H](C(=O)N(CCO)c2ccc(F)cc2Cl)CC1]

To a suspension of 200 mg (0.56 mmol) trans-4-({[4-(phenoxycarbonyl)-1H-imidazol-5-yl]carbonyl}amino)cyclohexane-carboxylic acid in 10 ml dichloromethane 148 μl (1.12 mmol) 1-chloro-N,N,2-trimethylprop-1-en-1-amine were added and the mixture was stirred at room temperature for 1 hour. 136 μl (1.68 mmol) pyridine and 106 mg (0.56 mmol) 2-[(2-chloro-4-fluorophenyl)amino]ethanol were added and the reaction was stirred at room temperature for 45 minutes. The reaction mixture was concentrated in vacuo to obtain the crude product, which was purified by flash column chromatography (dichloromethane/ethyl acetate-gradient) to give 209 mg of the title compound as a solid material.

LC-MS (Method 2): Rₜ=1.18 min; MS (ESIneg) m/z=527.3 [M+H]⁻.

¹H-NMR (400 MHz, DMSO-d₆), δ [ppm]=13.57 (br. s., 1H), 9.36 (br. s., 1H), 7.93 (s, 1H), 7.52-7.45 (m, 2H), 7.37-7.30 (m, 1H), 7.30-7.22 (m, 3H), 7.04 (td, 1H), 6.79 (dd, 1H), 5.28 (t, 1H), 4.16 (t, 2H), 3.75-3.63 (m, 1H), 3.42-3.36 (m, 2H), 2.27 (m, 1H), 1.95-1.86 (m, 4H), 1.48-1.22 (m, 4H).

### Intermediate I74

### phenyl 5-({trans-4-[(2-chlorophenyl)(ethyl)carbamoyl]cyclohexyl}carbamoyl)-1H-imidazole-4-carboxylate

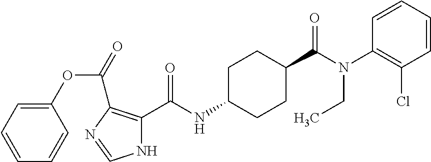 [SMILES: CCN(c1ccccc1Cl)C(=O)[C@H]1CC[C@H](NC(=O)c2[nH]cnc2C(=O)Oc2ccccc2)CC1]

To a suspension of 1.00 g (2.80 mmol) trans-4-({[4-(phenoxycarbonyl)-1H-imidazol-5-yl]carbonyl}amino)cyclohexane-carboxylic acid in 50 ml dichloromethane 555 μl (4.2 mmol) 1-chloro-N,N,2-trimethylprop-1-en-1-amine were added and the mixture was stirred at room temperature for 1 hour. 185 μl 1-chloro-N,N,2-trimethylprop-1-en-1-amine were added and the mixture was stirred for 30 minutes. 679 μl (8.39 mmol) pyridine and 435 mg (2.79 mmol) 2-chloro-N-ethylaniline were added and the reaction was stirred at room temperature for 45 minutes. The reaction mixture was concentrated in vacuo to obtain the crude product which was used without further purification in the subsequent steps.

### Intermediate I75

### N,N′-dimethyl-5,10-dioxo-5H,10H-diimidazo[1,5-a:1′,5′-d]pyrazine-1,6-dicarboxamide

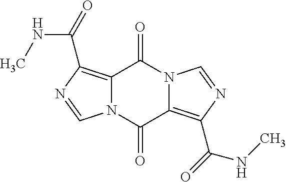

To 35 g (crude product) of 5,10-dioxo-5H,10H-diimidazo[1,5-a:1′,5′-d]pyrazine-1,6-dicarbonyl dichloride in 460 ml tetrahydrofuran were added dropwise 153 ml (307 mmol) of a 2M solution of methanamine in tetrahydrofuran and 68 ml (391 mmol)N-ethyl-N-isopropylpropan-2-amine. The resulting mixture was stirred for 20 h at room temperature.

The precipitate was filtered off and washed with dichloromethane. The obtained solid material was digested in methanol and dried in vacuum to give 15.2 g of the title compound as a crude product which was used without further purification in the subsequent steps.

### Intermediate I76

### ethyl4-{[tert-butyl(dimethyl)silyl]oxy}-1-(3-chloropropyl)cyclohexane carboxylate (mixture of cis-/trans-isomers)

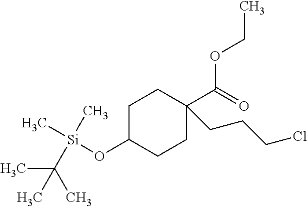 [SMILES: CCOC(=O)C1(CCCCl)CCC(O[Si](C)(C)C(C)(C)C)CC1]

Lithium diisopropylamide (10.5 ml, 21 mmol, 2 M solution in tetrahydrofuran) was added dropwise to a solution of ethyl 4-{[tert-butyl(dimethyl)silyl]oxy}cyclohexanecarboxylate (5.00 g, 17.5 mmol) in tetrahydrofuran (24 ml) at −78° C. and the mixture was stirred for 30 min at that temperature. 1-Bromo-2-chloroproane (2.6 ml, 26.2 mmol) was added and the mixture was stirred for 1 h at −78° C. The mixture was warmed during 2 h to room temperature. For work-up, water was added and the mixture was extracted with tert-butyl methyl ether (3×). The combined organic phases were washed with brine, filtrated through a silicone filter and concentrated under reduced pressure. The crude product was purified by flash chromatogryph (100 g Snap cartridge, hexanes/ethyl acetate gradient) to give ethyl4-{[tert-butyl(dimethyl)silyl]oxy}-1-(3-chloropropyl)cyclohexane carboxylate (4.19 g, as mixture of isomers, ratio 5:1 by GC-MS).

GC-MS (Method 10): Rₜ=21.20 min (minor isomer); 21.51 min (major isomer); MS (Cl) m/z=363.2 [M+H]⁺ and 382.2 [M+NH4]⁺.

### Intermediate I77

### 9-{[tert-butyl(dimethyl)silyl]oxy}-2-(2-chloro-4-fluorophenyl)-2-azaspiro[5.5]undecan-1-one

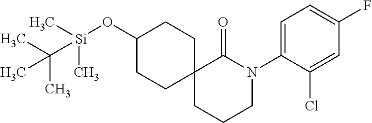 [SMILES: CC(C)(C)[Si](C)(C)OC1CCC2(CCCN(c3ccc(F)cc3Cl)C2=O)CC1]

Lithium hexamethyldisilazide (13.7 ml, 13.7 mmol, 1 M solution in tetrahydrofuran) was added over 5 min to solution of 2-chloro-4-fluoroaniline (1.10 g, 7.57 mmol, Cas No 2106-02-7) in tetrahydrofuran (45 ml) at −78° C. and the mixture was stirred at −78° C. for 1 h. A solution of ethyl4-{[tert-butyl(dimethyl)silyl]oxy}-1-(3-chloropropyl)cyclohexane carboxylate (mixture of cis-/trans-isomers) (2.50 g, 6.89 mmol) in tetrahydrofuran (30 ml) was added and the mixture was stirred for 2 h at −78° C. and then for 4 days at room temperature. For work-up, the reaction mixture was poured into a mixture of water and saturated sodium bicarbonate solution, extracted with ethyl acetate and the combined organic phases were washed with brine, filtrated through a silicone filter and concentrated under reduced pressured. The residue was purified by flash chromatography (hexanes/ethyl acetate gradient) to give 9-{[tert-butyl(dimethyl)silyl]oxy}-2-(2-chloro-4-fluorophenyl)-2-azaspiro[5.5]undecan-1-one in 2 fractions. Fraction 1 (700 mg, isomer 1) and fraction 2 (420 mg, isomer 2).

### Fraction 1 (Isomer 1):

¹H-NMR (400 MHz, DMSO-d₆): δ [ppm]=7.55 (dd, 1H), 7.41 (dd, 1H), 7.27 (td, 1H), 3.87 (br. s., 1H), 3.56-3.46 (m, 1H), 2.24-2.11 (m, 2H), 1.94-1.78 (m, 4H), 1.65-1.49 (m, 4H), 1.41-1.22 (m, 2H), 0.91-0.82 (m, 9H), 0.03 (s, 6H).

### Fraction 2 (Isomer 2):

¹H-NMR (400 MHz, DMSO-d₆): δ [ppm]=7.54 (dd, 1H), 7.42 (dd, 1H), 7.27 (td, 1H), 3.66-3.57 (m, 1H), 3.56-3.47 (m, 1H), 1.93-1.56 (m, 10H), 1.43-1.30 (m, 2H), 0.86 (s, 9H), 0.05 (s, 6H).

### Intermediate I78

### 2-(2-chloro-4-fluorophenyl)-9-hydroxy-2-azaspiro[5.5]undecan-1-one (isomer 1)

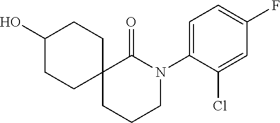 [SMILES: O=C1N(c2ccc(F)cc2Cl)CCCC12CCC(O)CC2]

Tetra-N-butylammonium fluoride (3.43 ml, 3.43 mmol, 1 M solution in tetrahyrofuran) was added to a solution of 9-{[tert-butyl(dimethyl)silyl]oxy}-2-(2-chloro-4-fluorophenyl)-2-azaspiro[5.5]undecan-1-one (isomer 1) (730 mg, fraction 1) in tetrahydrofuran (13 ml) and the mixture was stirred at room temperature for 18 h. Then tetra-N-butylammonium fluoride (3.43 ml, 3.43 mmol, 1 M solution in tetrahyrofuran) was added and the mixture was stirred at room temperature for 1 day. The mixture was concentrated under reduced pressure. The residue was purified by flash chromatography (dichloromethane/ethyl acetate gradient) to provide 2-(2-chloro-4-fluorophenyl)-9-hydroxy-2-azaspiro[5.5]undecan-1-one (isomer 1) (485 mg, 1.56 mmol).

¹H-NMR (400 MHz, DMSO-d₆): δ [ppm]=7.54 (dd, 1H), 7.42 (dd, 1H), 7.27 (td, 1H), 4.32 (d, 1H), 3.70-3.63 (m, 1H), 3.58-3.48 (m, 1H), 3.33-3.28 (m, 1H), 2.20-2.09 (m, 2H), 1.96-1.75 (m, 4H), 1.69-1.46 (m, 4H), 1.41-1.20 (m, 2H).

### Intermediate I79

### 2-(2-chloro-4-fluorophenyl)-9-hydroxy-2-azaspiro[5.5]undecan-1-one (isomer 2)

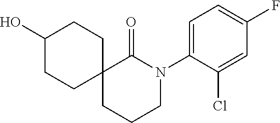 [SMILES: O=C1N(c2ccc(F)cc2Cl)CCCC12CCC(O)CC2]

Was prepared in analogy to the synthesis of 2-(2-chloro-4-fluorophenyl)-9-hydroxy-2-azaspiro[5.5]undecan-1-one (isomer 1) from 9-{[tert-butyl(dimethyl)silyl]oxy}-2-(2-chloro-4-fluorophenyl)-2-azaspiro[5.5]undecan-1-one (isomer 2) (445 mg, fraction 2) using tetra-N-butylammonium fluoride go give 2-(2-chloro-4-fluorophenyl)-9-hydroxy-2-azaspiro[5.5]undecan-1-one (isomer 2) (242 mg, 0.78 mmol).

¹H-NMR (400 MHz, DMSO-d₆): δ [ppm]=7.54 (dd, 1H), 7.41 (dd, 1H), 7.27 (td, 1H), 4.57 (d, 1H), 3.56-3.47 (m, 1H), 3.44-3.36 (m, 1H), 1.92-1.74 (m, 6H), 1.74-1.64 (m, 3H), 1.58 (dd, 1H), 1.38-1.22 (m, 2H).

### Intermediate I80

### 2-[2-(2-chloro-4-fluorophenyl)-1-oxo-2-azaspiro[5.5]undec-9-yl]-1H-isoindole-1,3(2H)-dione (isomer 1)

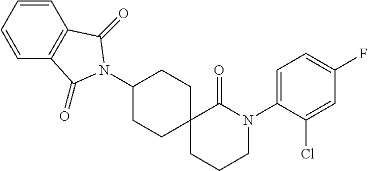 [SMILES: O=C1c2ccccc2C(=O)N1C1CCC2(CCCN(c3ccc(F)cc3Cl)C2=O)CC1]

Diisopropyl azodicarboxylate (0.46 ml, 1.03 mmol) was added dropwise to a mixture of 2-(2-chloro-4-fluorophenyl)-9-hydroxy-2-azaspiro[5.5]undecan-1-one (isomer 1) (480 mg, 1.54 mmol), phthalimide (340 mg, 2.31 mmol) and triphenylphosphine (606 mg, 2.31 mmol) in tetrahydrofuran (12 ml) and the mixture was stirred for 3 days at room temperature. For work-up, the reaction mixture was concentrated and the residue was purified by flash chromatography (hexanes/ethyl acetate gradient) to yield the 2-[2-(2-chloro-4-fluorophenyl)-1-oxo-2-azaspiro[5.5]undec-9-yl]-1H-isoindole-1,3(2H)-dione (isomer 1) (270 mg, 0.56 mmol)

LC-MS (Method 1): Rₜ=1.35 min; MS (ESIpos) m/z=441.2 [M+H]⁺.

¹H-NMR (400 MHz, DMSO-d₆): δ [ppm] 7.90-7.81 (m, 4H), 7.56 (dd, 1H), 7.45 (dd, 1H), 7.29 (td, 1H), 4.09-3.95 (m, 1H), 3.61-3.50 (m, 1H), 3.42-3.36 (m, 1H), 2.31-2.20 (m, 2H), 2.10-1.82 (m, 7H), 1.77 (d, 1H), 1.66-1.56 (m, 2H).

### Intermediate I81

### 9-amino-2-(2-chloro-4-fluorophenyl)-2-azaspiro[5.5]undecan-1-one (isomer 1)

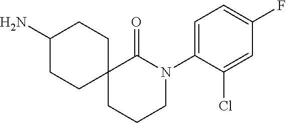 [SMILES: NC1CCC2(CCCN(c3ccc(F)cc3Cl)C2=O)CC1]

A mixture of 2-[2-(2-chloro-4-fluorophenyl)-1-oxo-2-azaspiro[5.5]undec-9-yl]-1H-isoindole-1,3(2H)-dione (isomer 1) (262 mg, 0.59 mmol) and hydrazine hydrate (146 μl) in ethanol (9 ml) was stirred at 80° C. for 3.5 h. The reaction mixture was concentrated under reduced pressure and the crude product was codestilled with dichloromethane (2×) and then used with out further purification in the next step.

### Intermediate I82

### Phenyl 5-{[2-(2-chloro-4-fluorophenyl)-1-oxo-2-azaspiro[5.5]undec-9-yl]carbamoyl}-1H-imidazole-4-carboxylate (isomer 1)

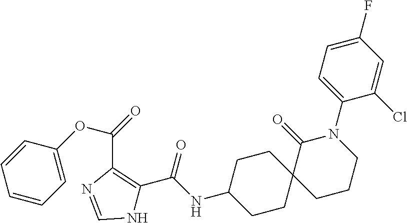 [SMILES: O=C(Oc1ccccc1)c1nc[nH]c1C(=O)NC1CCC2(CCCN(c3ccc(F)cc3Cl)C2=O)CC1]

To a suspension of 9-amino-2-(2-chloro-4-fluorophenyl)-2-azaspiro[5.5]undecan-1-one (isomer 1) (0.59 mmol) and diphenyl 5,10-dioxo-5H,10H-diimidazo[1,5-a:1′,5′-d]pyrazine-1,6-dicarboxylate (127 mg, 030 mmol) in 9 ml tetrahydrofuran were added 99 μl (0.71 mmol) triethylamine. After stirring for 2.5 h at 60° C. the precipitate was filtered of and the filtrate was used without further purification in the subsequent steps.

### Intermediate I83

### methyl 1,4-dioxaspiro[4.5]decane-8-carboxylate

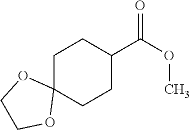

Methyl 4-oxocyclohexanecarboxylate (5.00 g, 32.0 mmol), ethylene glycol (5.4 ml, 96 mmol) and 4-methylbenzenesulfonic acid hydrate (609 mg, 3.20 mmol) were dissolved in 78 ml toluene and the mixture was stirred at 130° C. for 4 hours with a Dean-Stark apparatus. The mixture was cooled to room temperature and to the stirred mixture triethylamine (450 μl, 3.2 mmol) was added dropwise. The reaction was diluted with ethyl acetate and the organic phase was washed with sodium bicarbonate solution and brine, dried over sodium sulphate and concentrated under reduced pressure. The residue was purified by flash chromatography (hexanes/ethyl acetate gradient) to provide the title compound (2.6 g, 12.98 mmol).

### Intermediate I84

### methyl 1,4-dioxaspiro[4.5]decane-8-carboxylate

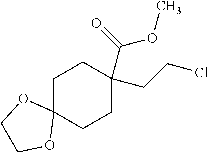 [SMILES: COC(=O)C1(CCCl)CCC2(CC1)OCCO2]

To a cooled solution of methyl 1,4-dioxaspiro[4.5]decane-8-carboxylate (2.50 g, 12.5 mmol) in 44 ml dry tetrahydrofuran was added lithium hexamethyldisilazide (16 ml, 1.0 M, 16 mmol) at −78° C. The mixture was stirred at −78° C. for 30 minutes and for 1 hour at 0° C. The reaction was cooled to −78° C. and 1-bromo-2-chloroethane (1.4 ml, 16 mmol) was added dropwise. The mixture was stirred for 1 hour at room temperature. For work-up, saturated ammonium chlorid solution was added and the mixture was poured into water. The mixture was extracted with ethyl acetate (3×). The combined organic phases were washed with brine, dried over sodium sulfate and concentrated under reduced pressure. Another batch was prepared accordingly (from 0.75 mmol methyl 1,4-dioxaspiro[4.5]decane-8-carboxylate) The combined crude products were purified by flash chromatogryph (hexanes/ethyl acetate gradient) to give methyl 8-(2-chloroethyl)-1,4-dioxaspiro[4.5]decane-8-carboxylate (2.5 g).

### Intermediate I85

### 10-(2-chloro-4-fluorophenyl)-1,4-dioxa-10-azadispiro[4.2.4.2]tetradecan-9-one

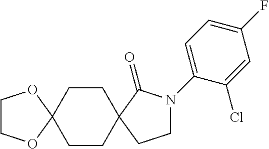 [SMILES: O=C1N(c2ccc(F)cc2Cl)CCC12CCC1(CC2)OCCO1]

Lithium hexamethyldisilazide (24 ml, 24 mmol, 1 M solution in tetrahydrofuran) was added dropwise to solution of 2-chloro-4-fluoroaniline (1.3 ml, 10 mmol, Cas No 2106-02-7) in tetrahydrofuran (40 ml) at −78° C. and the mixture was stirred at −78° C. for 30 minutes. A solution of methyl 1,4-dioxaspiro[4.5]decane-8-carboxylate (2.50 g, 9.52 mmol) in tetrahydrofuran (6 ml) was added and the mixture was stirred for 18 h at room temperature. For work-up, 20 ml methanol were added and the mixture was concentrated under reduced pressured. Another batch was prepared accordingly (0.38 mmol methyl 8-(2-chloroethyl)-1,4-dioxaspiro[4.5]decane-8-carboxylate) The combined crude products were purified by flash chromatogryphy (hexanes/ethyl acetate gradient) to give 10-(2-chloro-4-fluorophenyl)-1,4-dioxa-10-azadispiro[4.2.4.2]tetradecan-9-one (500 mg).

LC-MS (Method 2): Rₜ=1.11 min; MS (ESIpos) m/z=340.1 [M+H]⁺.

### Intermediate I86

### 2-(2-chloro-4-fluorophenyl)-2-azaspiro[4.5]decane-1,8-dione

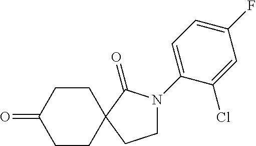 [SMILES: O=C1CCC2(CC1)CCN(c1ccc(F)cc1Cl)C2=O]

To a solution of 10-(2-chloro-4-fluorophenyl)-1,4-dioxa-10-azadispiro[4.2.4.2]tetradecan-9-one (500 mg, 1.47 mmol) in 6 ml acetone were added a 4 M-solution of hydrochloric acid in water (920 μl, 4.0 M, 3.7 mmol) and the mixture was stirred for 3 hours at 60° C. For work-up the reaction was poured into sat. sodiumbicarbonate solution and the mixture was extracted with dichloromethane (3×). The combined organic phases were washed with brine, dried over sodium sulfate and concentrated under reduced pressure. The crude product was purified by flash chromatogryph (hexanes/ethyl acetate gradient) to give 2-(2-chloro-4-fluorophenyl)-2-azaspiro[4.5]decane-1,8-dione (370 mg).

LC-MS (Method 1): Rₜ=0.95 min; MS (ESIpos) m/z=296.1 [M+H]⁺.

### Intermediate I87

### 8-(benzylamino)-2-(2-chloro-4-fluorophenyl)-2-azaspiro[4.5]decan-1-one

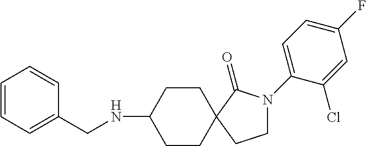 [SMILES: O=C1N(c2ccc(F)cc2Cl)CCC12CCC(NCc1ccccc1)CC2]

To a suspension of 2-(2-chloro-4-fluorophenyl)-2-azaspiro[4.5]decane-1,8-dione (370 mg, 1.25 mmol) in 13 ml dichloroethane were added benzylamine (140 μl, 99% Reinheit, 1.3 mmol) and sodium triacetoxyborhydride (410 mg, 1.88 mmol) and the mixture was stirred for 5 hours at room temperature. For work-up saturated sodium bicarbonate solution was added and the mixture was extracted with chloroform/2-propanol (9:1) filtrated through a silicone filter and concentrated under reduced pressure. The residue were washed with brine, dried over sodium sulfate and concentrated under reduced pressure to give 8-(benzylamino)-2-(2-chloro-4-fluorophenyl)-2-azaspiro[4.5]decan-1-one (500 mg) as crude product which was used without further purification in the subsequent steps.

LC-MS (Method 2): Rₜ=1.38 min; MS (ESIpos) m/z=389.3 [M+H]⁺.

### Intermediate I88

### 8-amino-2-(2-chloro-4-fluorophenyl)-2-azaspiro[4.5]decan-1-one

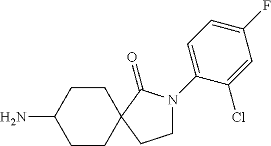 [SMILES: NC1CCC2(CC1)CCN(c1ccc(F)cc1Cl)C2=O]

8-(benzylamino)-2-(2-chloro-4-fluorophenyl)-2-azaspiro[4.5]decan-1-one (500 mg, 1.29 mmol) dissolved in methanol was added to a suspension of palladium(II)hydoxid (45 mg, 0.065 mmol, 20% on charcoal) in 7 ml methanol. The mixture was stirred under a hydrogen atmosphere at room temperature for 4 days. For work-up the catalyst was filtrated off, washed with methanol and the filtrate was concentrated under reduced pressure to give 8-amino-2-(2-chloro-4-fluorophenyl)-2-azaspiro[4.5]decan-1-one which was used without further purification.

### Intermediate I89

### sodium 6-methoxyimidazo[1,2-b]pyridazine-3-carboxylate

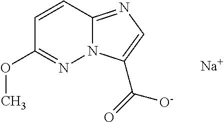

A mixture of 6-chloroimidazo[1,2-b]pyridazine-3-carboxylic acid (190 mg, 962 μmol, CAS No. 1208084-53-0) and sodium methanolate (660 μl, 25% in methanol, 2.9 mmol) in methanol (2.9 ml) was stirred at room temperature over night. The precipitate was collected by filtration, washed with methanol and dried to give a mixture of product and starting material. The mixture was resubmitted to similar reaction conditions to give the title compound as mixture with the starting material (185 mg, 3:1 by 1H NMR) which was used in the next step without further purification.

LC-MS (Method 1): Rₜ=0.49 min; MS (ESIpos) m/z=194.1 [M+H]⁺.

¹H-NMR (400 MHz, DMSO-d₆): δ [ppm]=7.90 (dd, 1H), 7.69-7.59 (m, 1H), 6.78 (dd, 1H), 3.93 (s, 3H).

### Intermediate I90

### 2-aminopyrazolo[1,5-a]pyrimidine-3-carboxylic acid lithium/and or sodium salt

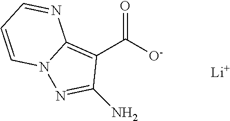 [SMILES: Nc1nn2cccnc2c1C(=O)[O-] ; [Li+]]

A mixture of ethyl 2-aminopyrazolo[1,5-a]pyrimidine-3-carboxylate (100 mg, 485 μmol, CAS No 1260169-02-5), lithium hydroxide (0.97 μl, 1 M aqueous solution, 0.97 mmol), tetrahydrofuran (3.2 ml) and methanol (0.97 ml) were stirred at room temperature over night. Then sodium hydroxide (0.65 ml, 2 M aqueous solution, 1.3 mmol) was added and the mixture was stirred for 24 h at room temperature. For work-up, the reaction mixture as concentrated, aceotroped twice with toluene and dried under high vacuum to give the title compound (lithium and/or sodium salt) as mixture with the starting material (105 mg) which was used in the next step without purification.

LC-MS (Method 1): Rₜ=0.52 min; MS (ESIpos) m/z=178.9 [M+H]⁺.

### Intermediate I91

### 2-chloro-N-ethyl-4-fluoroaniline

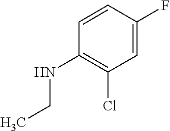

n-Butyllithium (2.7 ml, 2.5 M in hexanes, 6.9 mmol) was added to a solution of 2-chloro-4-fluoroaniline (1.00 g, 6.9 mmol) in tetrahydrofuran (8.9 ml) at −50° C. and the mixture was stirred at that temperature for 30 min. Bromoethane (510 μl, 6.9 mmol) was added and the mixture was stirred at −50° C. for 0.5 h and then warmed to room temperature. For workup, saturated ammonium chloride solution was added and the mixture was extracted with methyl tert-butyl ether. The combined organic phases were washed with brine, filtrated through a silicone filter and concentrated. The residue was purified by flash chromatography to give the title compound (417 mg, 34% yield).

LC-MS (Method 1): Rₜ=1.30 min; MS (ESIpos): m/z=174 [M+H]⁺ 1H-NMR (400 MHz, DMSO-d6) delta [ppm]: 1.136 (7.35), 1.154 (16.00), 1.172 (7.60), 2.523 (0.80), 3.086 (0.97), 3.104 (3.08), 3.118 (3.37), 3.122 (3.27), 3.136 (3.13), 3.153 (0.97), 5.044 (0.64), 5.059 (1.15), 5.073 (0.67), 6.659 (1.73), 6.673 (1.84), 6.682 (2.09), 6.695 (2.06), 6.997 (0.97), 7.004 (1.05), 7.018 (1.69), 7.026 (1.86), 7.041 (0.88), 7.048 (0.93), 7.219 (2.56), 7.227 (2.56), 7.241 (2.70), 7.248 (2.52).

### Intermediate I92

### 2-chloro-4-fluoro-N-methylaniline

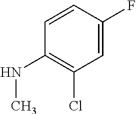

Was prepared in analogy to the synthesis of 2-chloro-N-ethyl-4-fluoroaniline using 2-chloro-4-fluoroaniline (1.00 g, 6.9 mmol) and iodomethane (430 μl, 6.9 mmol) as starting materials.

LC-MS (Method 1): Rₜ=1.17 min; MS (ESIpos): m/z=160 [M+H]⁺

¹H-NMR (400 MHz, DMSO-d₆): δ [ppm]=7.23 (dd, 1H), 7.04 (td, 1H), 6.60 (dd, 1H), 5.43-5.28 (m, 1H), 2.72 (d, 3H).

### Intermediate I93

### 2-chloro-4-fluoro-N,5-dimethylaniline

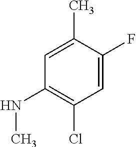

Was prepared in analogy to the synthesis of 2-chloro-N-ethyl-4-fluoroaniline using 2-chloro-4-fluoro-5-methylaniline (1.00 g, 6.3 mmol) and iodomethane (390 μl, 6.3 mmol) as starting materials.

¹H-NMR (400 MHz, DMSO-d₆): δ [ppm]=7.14 (d, 1H), 6.51 (d, 1H), 5.32-5.21 (m, 1H), 2.72 (d, 3H), 2.18 (d, 3H).

### Intermediate I94

### 2-chloro-N-(cyclopropylmethyl)-4-fluoroaniline

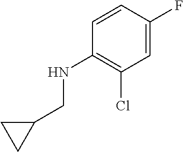 [SMILES: Fc1ccc(NCC2CC2)c(Cl)c1]

Was prepared in analogy to the synthesis of 2-chloro-N-ethyl-4-fluoroaniline using 2-chloro-4-fluoroaniline (1.00 g, 6.9 mmol) (bromomethyl)cyclopropane (670 μl, 6.9 mmol) and (bromomethyl)cyclopropane (670 μl, 6.9 mmol) as starting materials.

LC-MS (Method 1): Rₜ=1.38 min; MS (ESIpos): m/z=200 [M+H]⁺

¹H-NMR (400 MHz, DMSO-d₆): δ [ppm]=7.24 (dd, 1H), 7.02 (td, 1H), 6.73 (dd, 1H), 5.12-5.03 (m, 1H), 2.98 (t, 2H), 1.14-0.99 (m, 1H), 0.52-0.39 (m, 2H), 0.29-0.13 (m, 2H).

### Intermediate I95

### 2-(3-amino-4-chlorophenyl)propan-2-ol

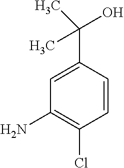

Methyl magnesium bromide (22 ml, 1 M, 22 mmol) was added dropwise to a solution of methyl 3-amino-4-chlorobenzoate (1.00 g, 5.39 mmol) in tetrahydrofuran (15 ml) at 0° C. and the mixture was warmed to room temperature and stirred for 24 h. Saturated ammonium chloride solution was added and the mixture was extracted with dichloromethane. The combined organic phases were filtrated through a silicone filter, and concentrated. The residue was purified by flash chromatography to give the title compound 55.0 mg (5% yield) LC-MS (Method 2): Rₜ=0.87 min; MS (ESIpos): m/z=186 [M+H]⁺

¹H-NMR (400 MHz, DMSO-d₆): δ [ppm]=7.07 (d, 1H), 6.94 (d, 1H), 6.61 (dd, 1H), 5.21 (s, 2H), 4.92 (s, 1H), 1.36 (s, 6H).

### Intermediate I96

### ethyl 1-methyl-4-({[5-(methylcarbamoyl)-1H-imidazol-4-yl]carbonyl}amino)cyclohexanecarboxylate (isomer 1)

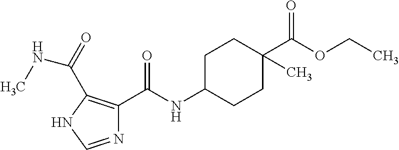 [SMILES: CCOC(=O)C1(C)CCC(NC(=O)c2nc[nH]c2C(=O)NC)CC1]

ethyl 4-amino-1-methylcyclohexanecarboxylate (371 mg, 2.00 mmol) and N-ethyl-N-isopropylpropan-2-amine (0.87 ml, 5.0 mmol) were added to a suspension of 5,10-dioxo-5H,10H-diimidazo[1,5-a:1′,5′-d]pyrazine-1,6-dicarbonyl dichloride (313 mg, 1.00 mmol) in tetrahydrofuran (16 ml) and the mixture was stirred at room temperature for 6 h. Then methanamine (1.0 ml, 2 M solution in tetrahydrofuran, 2.0 mmol) and N-ethyl-N-isopropylpropan-2-amine (0.52 ml, 3.0 mmol) in tetrahydrofuran (16 ml) were added and the mixture was stirred over night at room temperature. For work-up, insoluble material was filtrated off, the filtrate was concentrated and the residue was purified by flash chromatography (hexanes/ethyl acetate gradient) to give the title compound (513 mg).

LC-MS (Method 2): Rₜ=0.99 min; MS (ESIpos): m/z=337 [M+H]⁺

¹H-NMR (400 MHz, DMSO-d₆): δ [ppm]=13.35-12.94 (m, 1H), 11.30-10.67 (m, 1H), 8.74-8.32 (m, 1H), 7.80 (s, 1H), 4.13 (q, 2H), 3.81-3.54 (m, 1H), 2.80 (d, 3H), 2.16-2.04 (m, 2H), 1.90-1.68 (m, 2H), 1.35-1.26 (m, 2H), 1.21 (t, 3H), 1.13 (s, 3H).

### Intermediate I97

### 1-methyl-4-({[5-(methylcarbamoyl)-1H-imidazol-4-yl]carbonyl}amino)cyclohexanecarboxylic acid (isomer 1)

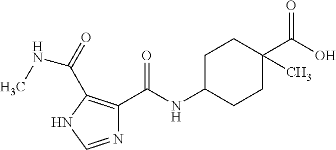 [SMILES: CNC(=O)c1[nH]cnc1C(=O)NC1CCC(C)(C(=O)O)CC1]

A mixture of ethyl cis-1-methyl-4-({[5-(methylcarbamoyl)-1H-imidazol-4-yl]carbonyl}amino)cyclohexanecarboxylate (510 mg, 1.52 mmol), lithium hydroxide (3.8 ml, 1 M aqueous solution), tetrahydrofuran (9.9 ml) and methanol (2.6 ml) were stirred at room temperature over night. Then sodium hydroxide (1.9 ml, 2 M aqueous solution, 3.8 mmol) was added and the mixture was stirred at 60° C. for 8 h. Upon cooling, the solvents were removed under reduced pressure, the residue was diluted with water and acidified using hydrochloric acid. The precipitate was collected by filtration, washed with water and ethanol and dried to give the title compound (350 mg).

LC-MS (Method 1): Rₜ=0.76 min; MS (ESIpos): m/z=309 [M+H]⁺

### Intermediate I98

### ethyl 1-methyl-4-[(pyrazolo[1,5-a]pyrimidin-3-ylcarbonyl)amino]cyclohexanecarboxylate (isomer 1)

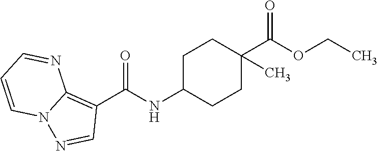 [SMILES: CCOC(=O)C1(C)CCC(NC(=O)c2cnn3cccnc23)CC1]

(Benzotriazol-1-yloxy)tripyrrolidinophosphonium hexafluorophosphate (572 mg, 1.10 mmol) was added to a mixture of pyrazolo[1,5-a]pyrimidine-3-carboxylic acid (163 mg, 1.00 mmol), ethyl 4-amino-1-methylcyclohexanecarboxylate (204 mg, 1.10 mmol), and N-ethyl-N-isopropylpropan-2-amine (0.70 ml, 4.0 mmol) in N,N-dimethylformamide (11 ml) and the mixture was stirred over night at room temperature. For work-up, the reaction mixture was concentrated and the residue was purified by flash chromatography to give the title compound (348 mg).

LC-MS (Method 2): Rₜ=1.04 min; MS (ESIpos): m/z=331 [M+H]⁺

### Intermediate I99

### 1-methyl-4-[(pyrazolo[1,5-a]pyrimidin-3-ylcarbonyl)amino]cyclohexanecarboxylic acid (isomer 1)

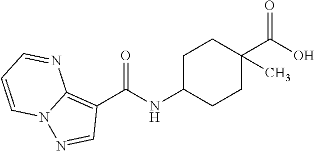 [SMILES: CC1(C(=O)O)CCC(NC(=O)c2cnn3cccnc23)CC1]

Was prepared in analogy to the synthesis of 1-methyl-4-({[5-(methylcarbamoyl)-1H-imidazol-4-yl]carbonyl}amino)cyclohexanecarboxylic acid (isomer 1) using ethyl 1-methyl-4-[(pyrazolo[1,5-a]pyrimidin-3-ylcarbonyl)amino]cyclohexanecarboxylate (isomer 1) (345 mg) as starting material to give the title compound (185 mg).

LC-MS (Method 1): Rₜ=0.75 min; MS (ESIpos): m/z=303 [M+H]⁺

1H-NMR (400 MHz, DMSO-d6) delta [ppm]: 1.106 (16.00), 1.143 (0.58), 1.162 (0.99), 1.180 (1.54), 1.198 (0.82), 1.235 (0.65), 1.262 (3.73), 1.286 (4.81), 1.305 (2.16), 1.340 (0.61), 1.819 (1.08), 1.831 (2.01), 1.838 (2.25), 1.841 (2.28), 1.850 (1.63), 1.864 (1.12), 1.884 (0.68), 2.042 (1.66), 2.049 (1.68), 2.062 (2.15), 2.065 (2.15), 2.071 (1.94), 2.100 (0.49), 2.305 (0.48), 2.647 (0.50), 3.638 (1.15), 3.809 (0.76), 4.094 (0.60), 4.112 (0.60), 7.226 (2.43), 7.237 (2.66), 7.244 (2.55), 7.250 (0.60), 7.255 (2.55), 7.721 (1.83), 7.740 (1.73), 8.534 (9.15), 8.541 (0.53), 8.778 (0.41), 8.784 (0.45), 8.791 (2.75), 8.795 (3.01), 8.802 (2.88), 8.806 (2.76), 9.269 (3.00), 9.274 (2.83), 9.287 (3.15), 9.291 (2.73).

### Intermediate I100

### ethyl 4-{[tert-butyl(dimethyl)silyl]oxy}-1-methylcyclohexanecarboxylate (mixture of cis-/trans-isomers)

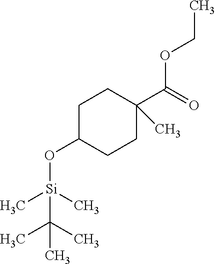 [SMILES: CCOC(=O)C1(C)CCC(O[Si](C)(C)C(C)(C)C)CC1]

Was prepared in analogy to the synthesis of ethyl 4-{[tert-butyl(dimethyl)silyl]oxy}-1-(2-chloroethyl)cyclohexanecarboxylate (mixture of cis-/trans-isomers) using ethyl 4-{[tert-butyl(dimethyl)silyl]oxy}cyclohexanecarboxylate (2.50 g, 8.73 mmol) and iodomethane (820 μl, 13 mmol) as starting materials. The crude product was purified by flash chromatography (100 g Snap Cartridge, hexanes/ethyl acetate gradient) to give the title compound as mixture of cis-/trans-isomers (1.27 g).

### Intermediate I101

### ethyl 4-hydroxy-1-methylcyclohexanecarboxylate (mixture of cis-/trans-isomers)

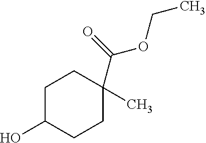

Tetra-N-butylammonium fluoride (8.5 ml, 8.5 mmol, 1 M solution in tetrahyrofuran) was added to a solution of ethyl 4-{[tert-butyl(dimethyl)silyl]oxy}-1-methylcyclohexanecarboxylate (mixture of cis-/trans-isomers) (1.27 g, 4.23 mmol) in tetrahydrofuran (40 ml) and the mixture was stirred at room temperature for 12 h. The mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (Snap cartridge, hexanes/ethyl acetate gradient, 20%->50% ethyl acetate) to provide the title compound as mixture of cis-/trans-isomers (518 mg).

¹H-NMR (400 MHz, DMSO-d₆, characteristic signals for both isomers): b [ppm]=4.48 (d, 0.75H), 4.44 (d, 0.25H), 4.13-4.02 (m, 2H), 3.61-3.45 (m, 0.25H), 1.11 (s, 1H), 1.07 (s, 2H).

### Intermediate I102

### ethyl 4-(1,3-dioxo-1,3-dihydro-2H-isoindol-2-yl)-1-methylcyclohexanecarboxylate (mixture of cis-/trans-isomers)

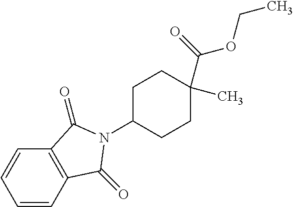 [SMILES: CCOC(=O)C1(C)CCC(N2C(=O)c3ccccc3C2=O)CC1]

Was prepared in analogy to the synthesis of 2-[2-(2-chloro-4-fluorophenyl)-1-oxo-2-azaspiro[4.5]dec-8-yl]-1H-isoindole-1,3(2H)-dione (isomer 1) using ethyl 4-hydroxy-1-methylcyclohexanecarboxylate (mixture of cis-/trans-isomers) (510 mg, 2.74 mmol) as starting material to give the title compound as mixture of cis-/trans-isomers (290 mg).

LC-MS (Method 1): Rₜ=1.31 min; MS (ESIpos): m/z=316 [M+H]⁺

¹H-NMR (400 MHz, DMSO-d₆, characteristic signals for both isomers): δ [ppm]=7.90-7.80 (m, 4H), 4.17 (q, 0.5H), 4.08 (q, 1.50H), 4.03-3.92 (m, 1H), 1.27 (s, 2H), 1.20 (t, 2.2H), 1.13 (s, 0.6H).

### Intermediate I103

### 4-(1,3-dioxo-1,3-dihydro-2H-isoindol-2-yl)-1-methylcyclohexanecarboxylic acid (mixture of cis-/trans-isomers)

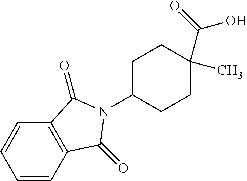 [SMILES: CC1(C(=O)O)CCC(N2C(=O)c3ccccc3C2=O)CC1]

A mixture of ethyl 4-(1,3-dioxo-1,3-dihydro-2H-isoindol-2-yl)-1-methylcyclohexanecarboxylate (mixture of cis-/trans-isomers) 285 mg, 0.904 mmol), lithium hydroxide (2.3 ml, 1 M aqueous solution, 2.3 mmol), tetrahydrofuran (5.9 ml) and methanol (1.6 ml) was stirred at room temperature over night, sodium hydroxide (1.1 ml, 2 M aqueous solution, 2.3 mmol) was added and the mixture was stirred at 60° C. for 8 h. For work-up, the reaction mixture was concentrated, then diluted with water and acidified with hydrochloric acid. The precipitate was collected by filtration, washed with water and ethanol and dried to give the crude product which was used in the next step without further purification (453 mg).

### Intermediate I104

### N-(2-chloro-4-fluorophenyl)-4-(1,3-dioxo-1,3-dihydro-2H-isoindol-2-yl)-1-methylcyclohexanecarboxamide (mixture of cis-/trans-isomers)

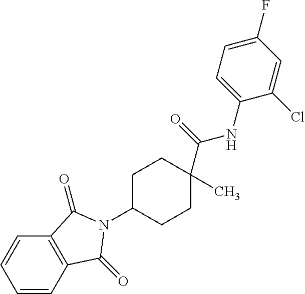 [SMILES: CC1(C(=O)Nc2ccc(F)cc2Cl)CCC(N2C(=O)c3ccccc3C2=O)CC1]

1-chloro-N,N,2-trimethylprop-1-en-1-amine (710 μl, 5.8 mmol) was added to a solution of 4-(1,3-dioxo-1,3-dihydro-2H-isoindol-2-yl)-1-methylcyclohexanecarboxylic acid (mixture of cis-/trans-isomers) (445 mg, 1.46 mmol) in dichloromethane (20 ml) and the mixture was stirred for 1 h. Pyridine (710 μl, 8.7 mmol) and 2-chloro-4-fluoroaniline (350 μl, 2.9 mmol) were added and the mixture was stirred for 16 h at room temperature and 4 h at 40° C. For work-up, aqueous sodium bicarbonate solution was added and the mixture was extracted with dichloromethane. The combined organic phases were filtrated through a silicone filter and concentrated. The residue was purified by flash chromatography (Snap Cartdrige, hexanes/ethyl acetate gradient) to give the title compound as mixture of cis-/trans-isomers (610 mg).

LC-MS (Method 2): Rₜ=1.29 min and 1.37 min; MS (ESIpos): m/z=415.3 [M+H]⁺

### Intermediate I105

### 4-amino-N-(2-chloro-4-fluorophenyl)-1-methylcyclohexanecarboxamide (mixture of cis-/trans-isomers)

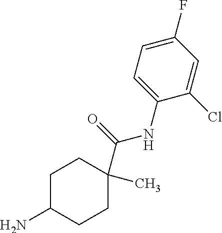 [SMILES: CC1(C(=O)Nc2ccc(F)cc2Cl)CCC(N)CC1]

A mixture of N-(2-chloro-4-fluorophenyl)-4-(1,3-dioxo-1,3-dihydro-2H-isoindol-2-yl)-1-methylcyclohexanecarboxamide (mixture of cis-/trans-isomers) (610 mg, 1.47 mmol) and hydrazine hydrate (360 μl, 7.4 mmol) in ethanol (13 ml) was stirred for 3 h at 80° C. Upon cooling, the precipitate was filtrated off, washed with ethanol and the filtrate was concentrated to give the title compound as mixture of isomers, which was used in the next step without further purification (308 mg).

### Intermediate I106

### Phenyl 5-{[trans-2-(2-chloro-4,5-difluorophenyl)-1-oxo-2-azaspiro[4.5]dec-8-yl]carbamoyl}-1H-imidazole-4-carboxylate (isomer 1)

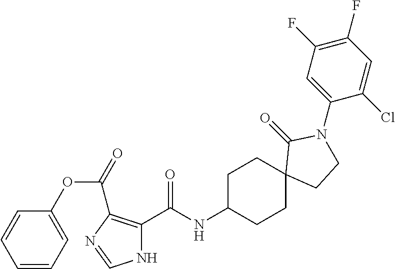 [SMILES: O=C(Oc1ccccc1)c1nc[nH]c1C(=O)NC1CCC2(CC1)CCN(c1cc(F)c(F)cc1Cl)C2=O]

To a mixture of trans-8-amino-2-(2-chloro-4,5-difluorophenyl)-2-azaspiro[4.5]decan-1-one (500 mg, 1.59 mmol) in dichloromethane (9.7 ml), was added diphenyl 5,10-dioxo-5H,10H-diimidazo[1,5-a:1′,5′-d]pyrazine-1,6-dicarboxylate (340 mg, 794 μmol) and triethylamine (220 μl). The mixture was stirred for 5 h at room temperature. For work-up, the product was concentrated and purified by flash chromatography to give the title compound (870 mg).

LC-MS (Method 1): Rₜ=1.21 min; MS (ESIpos) m/z=529.1 [M+H]⁺.

### Intermediate I107

### tert-butyl {trans-4-[(2-chloro-4-fluorophenyl)carbamoyl]cyclohexyl}carbamate

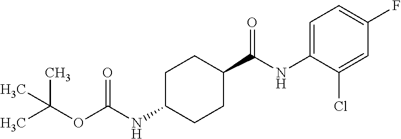 [SMILES: CC(C)(C)OC(=O)N[C@H]1CC[C@H](C(=O)Nc2ccc(F)cc2Cl)CC1]

The reaction was performed for 2 batches in parallel: to a solution of trans-4-(tert-butoxycarbonylamino)cyclohexane-1-carboxylic acid (25 g, 103 mmol) in tetrahydrofuran (500 mL) was added 2-chloro-4-fluoro-aniline (14.96 g, 103 mmol) and pyridine (163 g, 2.06 mol). Then phosphoryl chloride (94.2 g, 614 mmol) was added to the mixture at −20° C. The mixture was stirred at −20° C. for 1 h. Then the mixture was warmed to 15° C. slowly and stirred for another 3 h. TLC (petroleum ether/ethyl acetate=3:1) indicated the reaction completed. The reaction mixture was poured into water (2.5 l) and the mixture was extracted with ethyl acetate (2×1 l). The combined organic phases were washed with aqueous hydrochloric acid (1 M, 800 mL), water (1 I1) and saturated brine (800 ml). The organic phases of both bathes were combined and concentrated in vacuum to approx. 1/5 volume. The formed solid was filtered, washed with ethyl acetate and dried to give the first batch of trans-tert-butyl N-[4-[(2-chloro-4-fluoro-phenyl)carbamoyl]cyclohexyl] carbamate (30 g) as a white solid. The filtrate was concentrated in vacuum to give a yellow solid. The solid was recrystallized from ethyl acetate to give the second batch of trans-tert-butyl N-[4-[(2-chloro-4-fluoro-phenyl)carbamoyl]cyclohexyl] carbamate (10 g) as a white solid.

¹H-NMR (400 MHz, CDCl₃): δ [ppm]=8.35-8.31 (m, 1H), 7.55 (s, 1H), 7.15-7.12 (m, 1H), 7.03-7.01 (m, 1H), 4.45 (br. s, 1H), 3.25 (br. s, 1H), 2.25-2.05 (m, 5H), 1.70-1.67 (m, 2H), 1.46 (s, 9H), 1.21-1.18 (m, 2H).

### Intermediate I108

### trans-4-amino-N-(2-chloro-4-fluorophenyl)cyclohexanecarboxamide hydrochloric acid salt

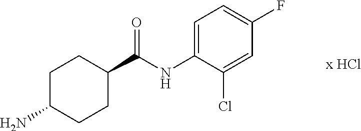 [SMILES: N[C@H]1CC[C@H](C(=O)Nc2ccc(F)cc2Cl)CC1]

To a solution of trans-tert-butyl N-[4-[(2-chloro-4-fluoro-phenyl)carbamoyl]cyclohexyl]carbamate (40 g, 108 mmol) in ethyl acetate (150 ml) was added hydrochloric acid (4 M solution in ethyl acetate, 250 ml) at 0° C. The mixture was stirred at 0° C. for 1 h. TLC (petroleum ether/ethyl acetate=3:1) indicated the reaction completed. The formed solid was collected by filtration, washed with ethyl acetate to give trans-4-amino-N-(2-chloro-4-fluorophenyl)cyclohexanecarboxamide hydrochloric acid salt (30.7 g, 93% yield) as a white solid.

¹H NMR (400 MHz, DMSO-d₆): δ [ppm]=9.58 (s, 1H), 8.16 (s, 3H), 7.59-7.56 (m, 1H), 7.50-7.47 (m, 1H), 7.23-7.21 (m, 1H), 2.99 (br. s, 1H), 2.50-2.45 (m, 1H), 2.03-1.90 (m, 4H), 1.50-1.39 (m, 4H).

### Intermediate I109

### 8-{[tert-butyl(dimethyl)silyl]oxy}-2-(4-fluorophenyl)-2-azaspiro[4.5]decan-1-one

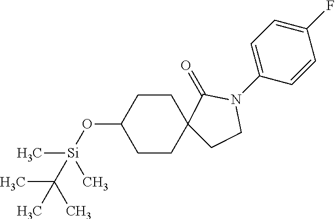 [SMILES: CC(C)(C)[Si](C)(C)OC1CCC2(CC1)CCN(c1ccc(F)cc1)C2=O]

Lithium hexamethyldisilazide (43 ml, 43 mmol, 2 M solution in tetrahydrofuran) was added drop wise over 20 min to a solution of 4-fluoroaniline (2.63 g, 23.6 mmol, CAS No 371-40-4) in tetrahydrofuran (190 ml) at −78° C. and the mixture was stirred for 1 h at that temperature. ethyl trans-4-{[tert-butyl(dimethyl)silyl]oxy}-1-(2-chloroethyl) cyclohexanecarboxylate (mixture of cis-/trans-isomers) (7.50 g, 21.5 mmol) was added drop wise during 1 h and the mixture was then stirred for 2 h at −78° C. A catalytic amount of potassium iodide was added and the mixture was warmed to room temperature and stirred overnight. For work-up, sodium bicarbonate solution was added and the mixture was extracted with ethyl acetate (3×). The combined organic phases were washed with brine, filtrated through a silicone filter and concentrated. The residue was purified by flash chromatography (340 g Snap Cartridge, hexanes/ethyl acetate gradient, 0%->10% ethyl acetate) to give 8-{[tert-butyl(dimethyl)silyl]oxy}-2-(4-fluorophenyl)-2-azaspiro[4.5]decan-1-one in 3 fractions: Fraction 1 (4.08 g, isomer 1), fraction 2 (0.66 g, mixture of isomer 1 and isomer 2,6:4), and fraction 3 (0.450 g, isomer 2).

### Fraction 1 (Isomer 1):

LC-MS (Method 2): Rₜ=1.77 min; MS (ESIpos) m/z=378.3 [M+H]⁺.

¹H-NMR (400 MHz, DMSO-d₆): δ [ppm]=7.76-7.63 (m, 1H), 7.25-7.16 (m, 1H), 3.99-3.92 (m, 1H), 3.75 (t, 2H), 2.04-1.88 (m, 4H), 1.70-1.52 (m, 6H), 1.32-1.20 (m, 2H), 0.89 (s, 9H), 0.05 (s, 6H)

### Fraction 3 (Isomer 2):

LC-MS (Method 2): Rₜ=1.74 min; MS (ESIpos) m/z=378.3 [M+H]⁺.

¹H-NMR (400 MHz, DMSO-d₆): δ [ppm]=7.75-7.63 (m, 2H), 7.26-7.13 (m, 2H), 3.76 (t, 2H), 3.68-3.56 (m, 1H), 2.02 (t, 2H), 1.84-1.72 (m, 2H), 1.65-1.47 (m, 4H), 1.43-1.26 (m, 2H), 0.86 (s, 9H), 0.06 (s, 6H)

### Intermediate I110

### 2-(4-fluorophenyl)-8-hydroxy-2-azaspiro[4.5]decan-1-one (isomer 1)

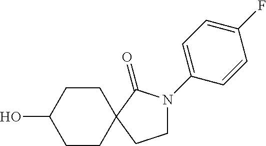 [SMILES: O=C1N(c2ccc(F)cc2)CCC12CCC(O)CC2]

Tetra-n-butyl ammonium fluoride (32 ml, 32 mmol, 1 M solution in tetrahyrofuran) was added to a solution of 8-{[tert-butyl(dimethyl)silyl]oxy}-2-(4-fluorophenyl)-2-azaspiro[4.5]decan-1-one (isomer 1) (4.07 g, 10.8 mmol) in tetrahydrofuran (93 ml) and the mixture was stirred at room temperature for 20 h. For work-up, the mixture was poured into water, extracted with ethyl acetate (3×) and the combined organic phases were washed with saturated sodium bicarbonate solution and brine, filtrated through a silicone filter and concentrated under reduced pressure. The residue was stirred with toluene, the precipitate formed was collected by filtration, washed with hexanes and dried to give the title compound (1.37 g). The mother liquor was concentrated and purified by flash chromatography (50 g snap cartridge, hexanes/ethyl acetate-gradient, 0->100% ethyl acetate) to give a second fraction of the title compound (1.08 g).

LC-MS (Method 2): Rₜ=0.96 min; MS (ESIneg): m/z=264.2 [M+H]⁺

¹H-NMR (400 MHz, DMSO-d6): δ [ppm]=7.74-7.65 (m, 2H), 7.26-7.16 (m, 2H), 4.40 (d, 1H), 3.80-3.71 (m, 3H), 2.03-1.88 (m, 4H), 1.73-1.61 (m, 2H), 1.59-1.49 (m, 2H), 1.30-1.20 (m, 2H)

### Intermediate I11

### 2-[2-(4-fluorophenyl)-1-oxo-2-azaspiro[4.5]dec-8-yl]-1H-isoindole-1,3(2H)-dione (isomer 1)

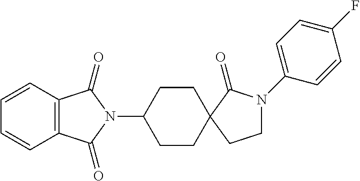 [SMILES: O=C1c2ccccc2C(=O)N1C1CCC2(CC1)CCN(c1ccc(F)cc1)C2=O]

Diisopropyl azodicarboxylate (1.5 ml, 7.8 mmol) was added drop wise to a mixture of 2-(4-fluorophenyl)-8-hydroxy-2-azaspiro[4.5]decan-1-one (isomer 1) (1.37 g, 5.02 mmol), phthalimide (1.15 g, 7.80 mmol) and triphenylphosphine (2.05 g, 7.80 mmol) in tetrahydrofuran (62 ml) and the mixture was stirred at room temperature overnight. For work-up, the reaction mixture was concentrated and the crude product was stirred with a mixture of ethyl acetate and methanol. The precipitate was collected by filtration, and dried to give the title compound (0.68 g).

LC-MS (Method 2): Rₜ=1.29 min; MS (ESIpos): m/z=393.3 [M+H]⁺

¹H-NMR (400 MHz, DMSO-d6): δ [ppm]=7.90-7.80 (m, 4H), 7.76-7.68 (m, 2H), 7.28-7.19 (m, 2H), 4.10-3.99 (m, 1H), 3.83 (t, 2H), 2.32-2.18 (m, 2H), 2.13 (t, 2H), 1.79-1.60 (m, 6H)

### Intermediate I112

### 8-amino-2-(4-fluorophenyl)-2-azaspiro[4.5]decan-1-one (isomer 1)

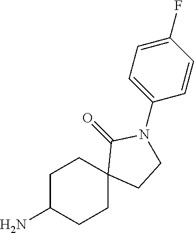 [SMILES: NC1CCC2(CC1)CCN(c1ccc(F)cc1)C2=O]

A mixture of 2-[2-(4-fluorophenyl)-1-oxo-2-azaspiro[4.5]dec-8-yl]-1H-isoindole-1,3(2H)-dione (isomer 1) (650 mg, 1.66 mmol) and hydrazine hydrate (410 μl, 8.3 mmol) in ethanol (14 ml) was stirred at 80° C. for 3.5 h. Upon cooling, the precipitate was filtrated off, washed with ethanol and the filtrate was concentrated to give the title compound (473 mg), which was used in the next step without further purification.

LC-MS (Method 2): Rₜ=0.92 min; MS (ESIneg): m/z=263.2 [M+H]⁻

¹H-NMR (400 MHz, DMSO-d6) δ[ppm]: 1.035 (0.96), 1.052 (2.14), 1.070 (1.47), 1.075 (1.34), 1.087 (1.34), 1.106 (3.66), 1.118 (3.61), 1.134 (3.91), 1.146 (3.78), 1.166 (1.89), 1.178 (1.83), 1.487 (1.53), 1.495 (1.75), 1.513 (11.79), 1.519 (13.89), 1.543 (5.00), 1.552 (5.79), 1.576 (1.26), 1.585 (1.40), 1.691 (4.46), 1.700 (4.54), 1.712 (2.46), 1.724 (4.21), 1.734 (3.86), 1.969 (8.64), 1.978 (1.78), 1.986 (14.80), 1.995 (1.80), 2.003 (8.99), 2.323 (0.73), 2.327 (0.99), 2.331 (0.74), 2.523 (3.96), 2.534 (4.15), 2.544 (2.18), 2.551 (1.29), 2.561 (1.95), 2.571 (0.98), 2.665 (0.82), 2.669 (1.10), 2.673 (0.85), 3.269 (2.76), 3.411 (0.98), 3.428 (1.31), 3.446 (1.20), 3.732 (9.69), 3.740 (1.89), 3.749 (15.42), 3.757 (1.95), 3.766 (9.36), 7.176 (0.73), 7.184 (9.43), 7.190 (2.88), 7.197 (1.29), 7.202 (3.56), 7.207 (16.00), 7.212 (3.80), 7.217 (1.51), 7.224 (3.03), 7.229 (10.48), 7.238 (0.96), 7.666 (0.87), 7.674 (10.20), 7.680 (3.37), 7.687 (10.70), 7.692 (5.38), 7.698 (9.95), 7.704 (3.23), 7.710 (9.38), 7.719 (0.95), 7.757 (1.66), 7.765 (1.48), 7.772 (1.51), 7.780 (1.94), 8.022 (2.00), 8.030 (1.53), 8.037 (1.50), 8.045 (1.61).

### Intermediate I113

### phenyl 4-{[2-(4-fluorophenyl)-1-oxo-2-azaspiro[4.5]dec-8-yl]carbamoyl}-1H-imidazole-5-carboxylate (isomer 1)

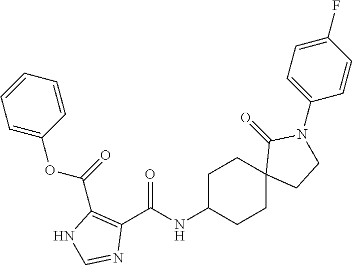 [SMILES: O=C(NC1CCC2(CC1)CCN(c1ccc(F)cc1)C2=O)c1nc[nH]c1C(=O)Oc1ccccc1]

A mixture of 8-amino-2-(4-fluorophenyl)-2-azaspiro[4.5]decan-1-one (isomer 1) (190 mg, 652 μmol), diphenyl 5,10-dioxo-5H,10H-diimidazo[1,5-a:1′,5′-d]pyrazine-1,6-dicarboxylate (140 mg, 326 μmol) and triethylamine (110 μl, 789 μmol) in tetrahydrofuran (10 ml) was stirred for 2.5 h at 60° C. The product was concentrated to give the title compound (0.47 g) which was used in the next step without further purification.

LC-MS (Method 2): Rₜ=0.95 min; MS (ESIneg): m/z=477.2 [M+H]⁺

### Intermediate I114

### 8-{[tert-butyl(dimethyl)silyl]oxy}-2-phenyl-2-azaspiro[4.5]decan-1-one (mixture of cis-/trans-isomers)

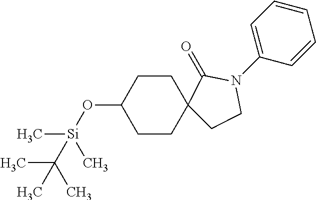 [SMILES: CC(C)(C)[Si](C)(C)OC1CCC2(CC1)CCN(c1ccccc1)C2=O]

Lithium bis(trimethylsilyl)amide (43 ml, 1.0 M in THF, 43 mmol) was added during 15 minutes drop wise to a solution of aniline (2.20 g, 23.6 mmol) in tetrahydrofuran (200 ml) at −78° C. and the mixture was stirred for 1 h at that temperature. A solution of ethyl 4-{[tert-butyl(dimethyl)silyl]oxy}-1-(2-chloroethyl)cyclohexanecarboxylate (mixture of cis-/trans-isomers) (7.50 g, 21.5 mmol) in tetrahydrofuran (200 ml) was added drop wise over 1 h and the mixture was stirred for 2 h at −78° C. The mixture was warmed to room temperature and a catalytic amount of potassium iodide was added and the mixture was warmed to room temperature and stirred overnight. For work-up, sodium bicarbonate solution was added and the mixture was extracted with ethyl acetate (3×). The combined organic phases were washed with brine, filtrated through a silicone filter and concentrated. The residue was purified by flash chromatography (340 g Snap Cartridge, hexanes/ethyl acetate gradient, 0%->15% ethyl acetate) to give 8-{[tert-butyl(dimethyl)silyl]oxy}-2-phenyl-2-azaspiro[4.5]decan-1-one in 3 fractions: Fraction 1 (4.22 g, isomer 1), fraction 2 (0.10 g, mixture of isomer 1 and isomer 2 ca. 7:3), and fraction 3 (0.720 g, isomer 2).

### Fraction 1 (Isomer 1):

¹H-NMR (400 MHz, DMSO-d₆): δ [ppm]=7.68-7.63 (m, 2H), 7.37-7.31 (m, 2H), 7.13-7.06 (m, 1H), 3.97-3.89 (m, 1H), 3.73 (t, 2H), 2.01-1.86 (m, 4H), 1.68-1.48 (m, 4H), 1.29-1.20 (m, 2H), 0.87 (s, 9H), 0.04 (s, 6H)

Fraction 3 (Isomer 2): LC-MS (Method 2): Rₜ=1.75 min; MS (ESIpos): m/z=360.3 [M+H]⁺

¹H-NMR (400 MHz, DMSO-d6): δ [ppm]=7.70-7.64 (m, 2H), 7.40-7.33 (m, 2H), 7.16-7.09 (m, 1H), 3.77 (t, 2H), 3.68-3.59 (m, 1H), 2.02 (t, 2H), 1.84-1.74 (m, 2H), 1.65-1.51 (m, 4H), 1.41-1.28 (m, 2H), 0.87 (s, 9H), 0.07 (s, 6H)

### Intermediate I115

### 8-hydroxy-2-phenyl-2-azaspiro[4.5]decan-1-one (isomer 1)

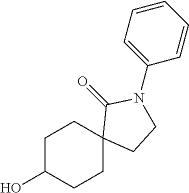 [SMILES: O=C1N(c2ccccc2)CCC12CCC(O)CC2]

Tetra-N-butylammonium fluoride (35 ml, 35 mmol, 1 M solution in tetrahyrofuran) was added to a solution 8-{[tert-butyl(dimethyl)silyl]oxy}-2-phenyl-2-azaspiro[4.5]decan-1-one (isomer 1) (4.22 g, 11.7 mmol) in tetrahydrofuran (100 ml) and the mixture was stirred at room temperature for 20 h. The mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography to give the title compound (2.57 g).

LC-MS (Method 2): Rₜ=0.94 min; MS (ESIpos) m/z=246.2 [M+H]⁺

¹H-NMR (400 MHz, DMSO-d6): δ [ppm]=7.70-7.65 (m, 2H), 7.40-7.34 (m, 2H), 7.15-7.09 (m, 1H), 4.40 (d, 1H), 3.80-3.72 (m, 3H), 2.02-1.89 (m, 4H), 1.73-1.62 (m, 2H), 1.60-1.49 (m, 2H), 1.30-1.21 (m, 2H)

### Intermediate I116

### 2-[1-oxo-2-phenyl-2-azaspiro[4.5]dec-8-yl]-1H-isoindole-1,3(2H)-dione (isomer 1)

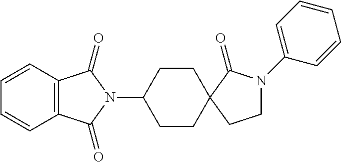 [SMILES: O=C1c2ccccc2C(=O)N1C1CCC2(CC1)CCN(c1ccccc1)C2=O]

Diisopropyl azodicarboxylate (3.1 ml, 16 mmol) was added drop wise to a mixture of 8-hydroxy-2-phenyl-2-azaspiro[4.5]decan-1-one (isomer 1) (2.57 g, 10.5 mmol), phthalimide (2.31 g, 15.7 mmol) and triphenylphosphine (4.12 g, 15.7 mmol) in tetrahydrofuran (120 ml) and the mixture was stirred at room temperature for 12 h. For work-up, the reaction mixture was concentrated and the residue was stirred with methanol. The precipitate was collected by filtration, washed with methanol and dried to give the title compound (1.55 g).

LC-MS (Method 2): Rₜ=1.29 min; MS (ESIpos): m/z=375.2 [M+H]⁺

¹H-NMR (600 MHz, DMSO-d6) δ[ppm]: 1.643 (1.29), 1.650 (1.67), 1.665 (4.54), 1.672 (5.19), 1.687 (5.97), 1.694 (10.51), 1.715 (5.81), 1.720 (6.64), 1.737 (4.60), 1.743 (3.98), 1.759 (0.83), 2.124 (6.62), 2.136 (11.32), 2.147 (6.91), 2.223 (1.34), 2.230 (1.72), 2.245 (3.87), 2.252 (4.25), 2.265 (4.01), 2.273 (3.87), 2.287 (1.43), 2.294 (1.29), 2.381 (1.05), 2.384 (1.43), 2.387 (1.08), 2.482 (3.87), 2.486 (5.43), 2.515 (4.28), 2.518 (4.57), 2.521 (5.00), 2.526 (3.04), 2.609 (1.05), 2.612 (1.37), 2.615 (0.99), 3.292 (3.58), 3.330 (2.93), 3.332 (2.02), 3.821 (7.07), 3.827 (2.21), 3.832 (12.83), 3.838 (1.94), 3.844 (6.96), 4.022 (0.83), 4.029 (1.67), 4.035 (1.05), 4.043 (1.83), 4.050 (3.20), 4.056 (1.77), 4.064 (1.02), 4.070 (1.56), 4.077 (0.81), 7.123 (1.86), 7.125 (3.04), 7.127 (2.07), 7.135 (4.17), 7.137 (6.67), 7.148 (2.61), 7.149 (3.60), 7.151 (2.42), 7.364 (1.48), 7.367 (7.34), 7.371 (3.04), 7.379 (10.03), 7.382 (10.08), 7.391 (3.07), 7.394 (7.26), 7.687 (10.49), 7.689 (11.19), 7.698 (4.22), 7.701 (10.81), 7.703 (8.61), 7.831 (4.95), 7.835 (5.08), 7.837 (6.10), 7.838 (6.13), 7.841 (7.07), 7.845 (15.35), 7.850 (7.37), 7.851 (7.72), 7.856 (16.00), 7.860 (7.64), 7.862 (5.89), 7.864 (6.10), 7.865 (5.24), 7.870 (5.08).

### Intermediate I117

### 8-amino-2-phenyl-2-azaspiro[4.5]decan-1-one (isomer 1)

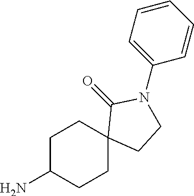 [SMILES: NC1CCC2(CC1)CCN(c1ccccc1)C2=O]

A mixture of 2-[1-oxo-2-phenyl-2-azaspiro[4.5]dec-8-yl]-1H-isoindole-1,3(2H)-dione (isomer 1) (1.52 g, 4.06 mmol) and hydrazine hydrate (1.0 ml, 20 mmol) in ethanol (35 ml) was stirred at 80° C. for 4 h. Upon cooling, the precipitate was filtrated off, washed with ethanol and the filtrate was concentrated and the residue was stirred with dichloromethane. The solid was filtrated off and the filtrated was washed with water and brine, filtrated through a silicone filter and concentrated to give the title compound (958 mg), which was used for the next step without further purification.

LC-MS (Method 2): Rₜ=0.89 min; MS (ESIpos): m/z=245 [M+H]⁺

¹H-NMR (400 MHz, DMSO-d6) δ[ppm]: 1.064 (1.39), 1.076 (1.43), 1.096 (3.87), 1.107 (3.73), 1.124 (4.08), 1.135 (4.07), 1.156 (2.00), 1.168 (2.05), 1.486 (3.05), 1.496 (3.28), 1.517 (15.63), 1.526 (12.73), 1.548 (6.21), 1.557 (6.92), 1.581 (1.83), 1.590 (1.97), 1.686 (4.76), 1.695 (4.69), 1.707 (2.54), 1.719 (4.39), 1.729 (3.95), 1.974 (8.96), 1.983 (1.90), 1.991 (15.09), 2.000 (1.77), 2.009 (9.33), 2.323 (0.68), 2.327 (0.92), 2.523 (3.98), 2.541 (1.91), 2.665 (0.69), 2.669 (0.92), 2.674 (0.68), 3.742 (10.14), 3.751 (1.87), 3.759 (16.00), 3.767 (2.00), 3.776 (9.73), 5.760 (4.33), 7.096 (2.00), 7.098 (3.51), 7.101 (1.94), 7.117 (7.97), 7.132 (2.99), 7.135 (4.69), 7.138 (2.57), 7.336 (1.20), 7.341 (9.36), 7.346 (3.36), 7.354 (1.91), 7.360 (11.38), 7.363 (12.49), 7.368 (2.16), 7.376 (3.14), 7.381 (9.20), 7.386 (1.46), 7.652 (1.46), 7.658 (12.44), 7.660 (13.69), 7.663 (7.17), 7.674 (3.70), 7.679 (13.01), 7.683 (9.56), 7.687 (1.93).

### Intermediate I118

### ethyl 4-{[tert-butyl(dimethyl)silyl]oxy}-1-(2-methoxyethyl)cyclohexanecarboxylate

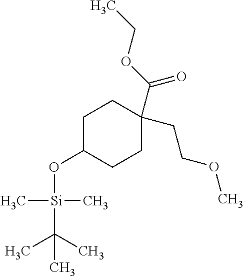

A solution of ethyl 4-{[tert-butyl(dimethyl)silyl]oxy}cyclohexanecarboxylate (mixture of cis-/trans-isomers) (7.50 g, 26.2 mmol) in tetrahydrofuran (59 ml) was added over 25 min to a solution of lithium diisopropylamine (35 ml, 2 M solution in tetrahyrofuran, 71 mmol) in tetrahydrofuran (39 ml) at −5° C. and the mixture was stirred for 2.5 h at that temperature. 2-Bromoethyl methyl ether (6.6 ml, 71 mmol, CAS No 6482-24-2) was added over 20 min and the mixture was stirred for 2 h at room temperature. For work-up, water was added and the mixture was extracted with ethyl acetate (2×) and the combined organic phases were washed with brine, filtrated through a silicone filter and concentrated. The residue was purified by flash chromatography (340 g Snap Cartridge, hexanes/ethyl acetate gradient, 0%->10% ethyl acetate) to give ethyl 4-{[tert-butyl(dimethyl)silyl]oxy}-1-(2-methoxyethyl)cyclohexanecarboxylate as mixture of isomers: Fraction 1 (5.30 g, cis/trans ca. 3.3:1, based on ¹H NMR); Fraction 2 (2.10 g, cis-isomer based on ¹H NMR).

### Fraction 1 (Mixture of Cis-/Trans-Isomers):

¹H-NMR (400 MHz, DMSO-d₆, characteristic signals trans-isomer): δ [ppm]=3.88-3.82 (m, 1H), 1.52-1.41 (m, 4H), 0.87 (s, 6H)

### Fraction 2 (Cis-Isomer):

¹H-NMR (400 MHz, DMSO-d₆): δ [ppm]=4.08 (q, 1H), 3.64-3.53 (m, 1H), 3.25 (t, 2H), 3.15 (s, 3H), 2.10-1.97 (br. s., 2H), 1.76-1.51 (m, 4H), 1.28-1.12 (m, 7H), 0.84 (s, 9H), 0.02 (s, 9H)

### Intermediate I119

### ethyl cis-4-hydroxy-1-(2-methoxyethyl)cyclohexanecarboxylate

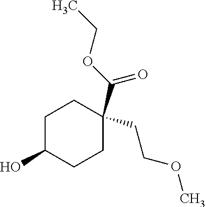 [SMILES: CCOC(=O)[C@]1(CCOC)CC[C@H](O)CC1]

Tetra-n-butylammonium fluoride (17 ml, 1 M solution in tetrahyrofuran, 17 mmol) was added to a solution of ethyl cis-4-{[tert-butyl(dimethyl)silyl]oxy}-1-(2-methoxyethyl)cyclohexanecarboxylate (fraction 2) (2.00 g, 5.80 mmol) in tetrahydrofuran (50 ml) and the mixture was stirred at room temperature for 20 h. For work-up, the mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (50 g Snap cartridge, hexanes/ethyl acetate gradient, 40%->90% ethyl acetate) to give the title compound (1.15 g).

¹H-NMR (400 MHz, DMSO-d6): δ [ppm]=4.48 (d, 1H), 4.08 (q, 2H), 3.39-3.30 (m, 1H), 3.25 (t, 2H), 3.15 (s, 3H), 2.11-1.99 (m, 2H), 1.89-1.49 (m, 5H), 1.22-1.02 (m, 7H)

### Intermediate I120

### ethyl 4-hydroxy-1-(2-methoxyethyl)cyclohexanecarboxylate (mixture of cis-/trans isomers)

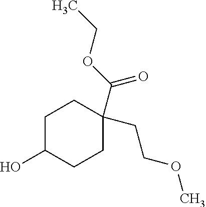 [SMILES: CCOC(=O)C1(CCOC)CCC(O)CC1]

Was prepared in analogy to the synthesis of ethyl cis-4-hydroxy-1-(2-methoxyethyl)cyclohexanecarboxylate using ethyl 4-{[tert-butyl (dimethyl) silyl]oxy}-1-(2-methoxyethyl)cyclohexanecarboxylate (mixture of cis-/trans-isomers) (4.10 g, 1.9 mmol, cis/trans: ca 3.3:1 based on ¹H NMR) as starting material to give ethyl 4-hydroxy-1-(2-methoxyethyl)cyclohexanecarboxylate (mixture of cis-/trans isomers) (2.27 g, 81% yield, ca. 2.8:1 cis-/trans-mixture based on ¹H NMR).

¹H-NMR (400 MHz, DMSO-d6, only characteristic signals trans-isomer): b [ppm]=4.41 (d, 1H), 3.65-3.56 (m, 1H), 3.16 (s, 3H)

### Intermediate I121

### 2-(2-chloro-4,6-difluorophenyl)-8-hydroxy-2-azaspiro[4.5]decan-1-one (isomer 1)

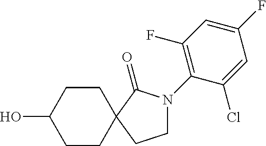 [SMILES: O=C1N(c2c(F)cc(F)cc2Cl)CCC12CCC(O)CC2]

A mixture of ethyl 4-hydroxy-1-(2-methoxyethyl)cyclohexanecarboxylate (mixture of cis-/trans-isomers) (2.50 g, 10.9 mmol), 2-chloro-4,6-difluoroaniline (1.95 g, 11.9 mmol, CAS No 36556-56-6) and dimethylaluminiumchloride (22 ml, 1 M solution in hexane, 22 mmol) in toluene (57 ml), was stirred under argon atmosphere for 4 h at room temperature followed by 6 h under reflux. For work-up, the mixture was poured into ice water, extracted with ethyl acetate (3×) and the combined organic phases were washed with brine, filtrated through a silicone filter and concentrated under reduced pressure. The residue was purified by flash chromatography (25 g Snap cartridge, hexanes/ethyl acetate gradient, 20%->50% ethyl acetate) to give the title compound (2.31 g) as single isomer (isomer 1).

LC-MS (Method 2): Rₜ=1.01 min; MS (ESIpos): m/z=316.1 [M+H]⁺

¹H-NMR (400 MHz, DMSO-d6): δ [ppm]=7.57-7.46 (m, 2H), 4.41 (d, 1H), 3.78-3.68 (m, 1H), 3.64-3.55 (m, 1H), 3.52-3.43 (m, 1H), 2.07 (t, 2H), 1.97-1.87 (m, 2H), 1.74-1.61 (m, 2H), 1.60-1.47 (m, 2H), 1.34-1.22 (m, 2H)

### Intermediate I122

### 2-[2-(2-chloro-4,6-difluorophenyl)-1-oxo-2-azaspiro[4.5]dec-8-yl]-1H-isoindole-1,3(2H)-dione (isomer 1)

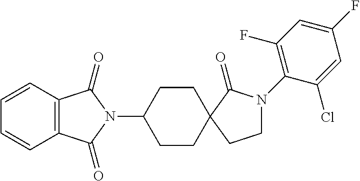 [SMILES: O=C1c2ccccc2C(=O)N1C1CCC2(CC1)CCN(c1c(F)cc(F)cc1Cl)C2=O]

Diisopropyl azodicarboxylate (2.2 ml, 11 mmol) was added drop wise to a mixture of 2-(2-chloro-4,6-difluorophenyl)-8-hydroxy-2-azaspiro[4.5]decan-1-one (isomer 1) (2.31 g, 7.32 mmol), phthalimide (1.61 g, 11.0 mmol, CAS No 85-41-6) and triphenylphosphine (2.88 g, 11.0 mmol) in tetrahydrofuran (87 ml) and the mixture was stirred for 12 h at room temperature. For work-up, the reaction mixture was concentrated and the residue was purified by flash chromatography (100 g Snap cartridge, hexanes/ethyl acetate gradient, 0%->50% ethyl acetate) to give the title compound (38 mg) as single isomer based on ¹H NMR.

LC-MS (Method 2): Rₜ=1.34 min; MS (ESIpos): m/z=445.2 [M+H]⁺

¹H-NMR (400 MHz, DMSO-d6) δ[ppm]: 1.658 (1.11), 1.669 (2.14), 1.679 (1.46), 1.701 (3.50), 1.710 (3.71), 1.722 (3.83), 1.752 (2.68), 2.221 (2.53), 2.224 (2.57), 2.240 (4.74), 2.258 (4.04), 2.281 (1.52), 2.293 (1.46), 2.322 (1.19), 2.327 (1.52), 2.332 (1.01), 2.518 (4.43), 2.523 (2.76), 2.539 (0.91), 2.664 (0.86), 2.669 (1.26), 2.673 (0.91), 3.549 (1.63), 3.555 (1.38), 3.566 (1.07), 3.573 (1.94), 3.590 (0.99), 3.643 (1.05), 3.658 (1.75), 3.661 (1.54), 3.667 (1.15), 3.677 (1.38), 3.682 (1.54), 4.049 (1.30), 7.506 (1.13), 7.513 (1.67), 7.528 (1.50), 7.537 (2.90), 7.544 (2.41), 7.547 (2.08), 7.551 (1.46), 7.554 (1.85), 7.559 (2.47), 7.565 (2.47), 7.569 (2.02), 7.572 (1.11), 7.576 (1.44), 7.824 (1.85), 7.830 (1.26), 7.835 (3.58), 7.841 (3.79), 7.846 (14.58), 7.853 (16.00), 7.859 (5.25), 7.865 (3.67), 7.870 (1.32), 7.876 (1.79).

### Intermediate I123

### 8-amino-2-(2-chloro-4,6-difluorophenyl)-2-azaspiro[4.5]decan-1-one (isomer 1)

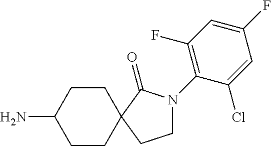 [SMILES: NC1CCC2(CC1)CCN(c1c(F)cc(F)cc1Cl)C2=O]

A mixture of 2-[2-(2-chloro-4,6-difluorophenyl)-1-oxo-2-azaspiro[4.5]dec-8-yl]-1H-isoindole-1,3(2H)-dione (isomer 1) (4.40 g, 9.89 mmol) and methanamine (7.1 ml, 40% aqueous solution, 99 mmol, CAS No 74-89-5) in ethanol (25 ml, 430 mmol), was stirred for 3 h at 60° C. For work-up, the reaction mixture was concentrated under reduced pressure and the residue was stirred with acetonitrile. The precipitate formed was collected by filtration, stirred with a mixture of dichloromethane and methanol (4:1) and precipitate was collected by filtration and dried to give the title compound (627 mg, 55% purity by LC-MS) which was used in the next step without further purification.

LC-MS (Method 2): Rₜ=0.96 min; MS (ESIpos): m/z=315.1 [M+H]⁺

### Intermediate I124

### ethyl 4-{[tert-butyl(dimethyl)silyl]oxy}-1-(cyanomethyl)cyclohexanecarboxylate (mixture of cis-/trans-isomers)

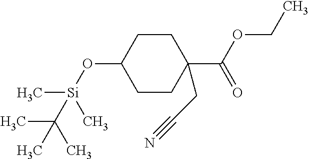 [SMILES: CCOC(=O)C1(CC#N)CCC(O[Si](C)(C)C(C)(C)C)CC1]

Lithium diisopropylamide (21 ml, 2 M solution in tetrahydrofuran, 42 mmol) was added drop wise to a solution of ethyl 4-{[tert-butyl(dimethyl)silyl]oxy}cyclohexanecarboxylate (mixture of cis-/trans-isomers) (13.2 g, 46.1 mmol) in tetrahydrofuran (62 ml) at −73° C. and the mixture was stirred for 30 min at that temperature. bromoacetonitrile (4.8 ml, 69 mmol, CAS No 590-17-0) was added drop wise and the mixture was then stirred for 1 h at the same temperature and then warmed to room temperature over 2.5 h. For work-up, water was added and the mixture was extracted with ethyl acetate (3×) and the combined organic phases were washed with brine, filtrated through a silicone filter and concentrated. The residue was purified by flash chromatography (340 g Snap Cartridge, hexanes/ethyl acetate gradient, 10%->18% ethyl acetate) to give the title compound in 3 fractions: fraction 1 (0.33 g, 2% yield, single isomer based on ¹H NMR, isomer 2), fraction 2 (4.26 g, 28% yield, mixture of isomer 1 and isomer 2, ca. 9:1 based on ¹H NMR), fraction 3 (3.17 g, 21% yield, single isomer based on ¹H NMR, isomer 1).

### Fraction 1 (Isomer 2):

¹H-NMR (400 MHz, DMSO-d₆): δ [ppm]=4.14 (q, 2H), 3.87-3.80 (m, 1H), 2.80 (s, 2H), 1.87-1.67 (m, 4H), 1.62-1.42 (m, 4H), 1.20 (t, 3H), 0.86 (s, 9H), 0.04 (s, 6H)

### Fraction 3 (Isomer 1):

¹H-NMR (400 MHz, DMSO-d₆): δ [ppm]=4.15 (q, 2H), 3.71-3.62 (m, 1H), 2.78 (s, 2H), 2.16-2.04 (m, 2H), 1.76-1.66 (m, 2H), 1.44-1.25 (m, 4H), 1.21 (t, 3H), 0.85 (s, 9H), 0.03 (s, 6H)

### Intermediate I125

### 8-{[tert-butyl(dimethyl)silyl]oxy}-2-azaspiro[4.5]decan-1-one (isomer 1)

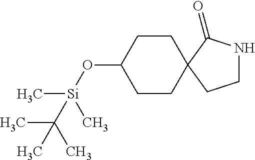 [SMILES: CC(C)(C)[Si](C)(C)OC1CCC2(CCNC2=O)CC1]

An autoclave was charged with a mixture of ethyl 4-{[tert-butyl(dimethyl)silyl]oxy}-1-(cyanomethyl)cyclohexanecarboxylate (mixture of cis-/trans-isomers) (7.40 g, 22.7 mmol), Raney-Nickel catalyst (4.67 g) and ammonia (102 ml, 2 M in ethanol, 204 mmol) and then pressurized with hydrogen (30 bar). The mixture was stirred at 40° C. over night. Upon cooling, the catalyst was filtrated off, washed with ethanol and the filtrate was concentrated under reduced pressure. The residue was dissolved in toluene (59 ml), triethylamine (6 ml) was added and the mixture was refluxed for 24 h. For work-up, the reaction mixture was concentrated and the residue was purified by flash chromatography (hexanes/ethyl acetate gradient) to give the title compound (4.40 g) as single isomer.

¹H-NMR (400 MHz, DMSO-d6): δ [ppm]=7.41 (br. s., 1H), 3.93-3.84 (m, 1H), 3.09 (t, 2H), 1.89-1.75 (m, 4H), 1.62-1.39 (m, 4H), 1.13-1.03 (m, 2H), 0.85 (s, 9H), 0.01 (s, 6H)

### Intermediate I126

### 8-{[tert-butyl(dimethyl)silyl]oxy}-2-(3,5-difluorophenyl)-2-azaspiro[4.5]decan-1-one (isomer 1)

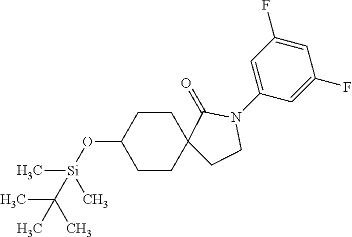 [SMILES: CC(C)(C)[Si](C)(C)OC1CCC2(CC1)CCN(c1cc(F)cc(F)c1)C2=O]

A mixture of 8-{[tert-butyl(dimethyl)silyl]oxy}-2-azaspiro[4.5]decan-1-one (isomer 1) (425 mg, 1.50 mmol), 1,3-difluoro-5-iodobenzene (360 mg, 1.50 mmol, CAS No 2265-91-0), copper(I) iodide (143 mg, 0.750 mmol), N,N′-dimethylethylenediamine (164 μl, 1.50 mmol) and potassium carbonate (435 mg, 3.15 mmol) in dioxane (26 ml) was heated to 160° C. for 2 h in a microwave reactor. Upon cooling, the reaction mixture was filtrated through a pad of celite, and the filtrate was concentrated and the residue was purified by flash chromatography (25 g Snap Cartridge, hexanes/ethyl acetate-gradient, 0%->50% ethyl acetate) to give the title compound as single isomer (575 mg).

LC-MS (Method 2): Rₜ=1.79 min; MS (ESIpos): m/z=396.3 [M+H]⁺

¹H-NMR (400 MHz, DMSO-d6): δ [ppm]=7.50-7.41 (m, 2H), 6.98-6.91 (m, 1H), 3.94-3.88 (m, 1H), 3.72 (t, 2H), 2.00-1.85 (m, 4H), 1.63-1.47 (m, 4H), 1.28-1.20 (m, 2H), 0.85 (s, 9H), 0.01 (s, 6H)

### Intermediate I127

### 2-(3,5-difluorophenyl)-8-hydroxy-2-azaspiro[4.5]decan-1-one (isomer 1)

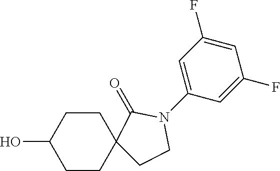 [SMILES: O=C1N(c2cc(F)cc(F)c2)CCC12CCC(O)CC2]

Tetra-N-butylammonium fluoride (4.4 ml, 1 M solution in tetrahydrofuran, 4.4 mmol) was added to a solution of 8-{[tert-butyl(dimethyl)silyl]oxy}-2-(3,5-difluorophenyl)-2-azaspiro[4.5]decan-1-one (isomer 1) (575 mg, 1.45 mmol) in tetrahydrofuran (12 ml) and the mixture was stirred at room temperature for 20 h. The mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (25 g Snap cartridge, hexanes/ethyl acetate-gradient, 0%->100% ethyl acetate) to give the title compound (360 mg) as single isomer.

LC-MS (Method 1): Rₜ=1.05 min; MS (ESIpos): m/z=282.1 [M+H]⁺

¹H-NMR (400 MHz, DMSO-d6) δ[ppm]: 1.172 (0.92), 1.225 (2.23), 1.236 (4.58), 1.246 (2.71), 1.258 (2.69), 1.269 (4.97), 1.279 (2.62), 1.493 (1.16), 1.501 (1.88), 1.510 (1.33), 1.534 (4.19), 1.555 (2.58), 1.564 (3.63), 1.572 (2.07), 1.623 (1.63), 1.635 (4.43), 1.647 (4.73), 1.658 (2.80), 1.669 (2.91), 1.681 (2.69), 1.693 (1.00), 1.892 (3.17), 1.902 (3.43), 1.925 (4.73), 1.932 (4.38), 1.954 (3.20), 1.963 (8.89), 1.980 (12.37), 1.988 (3.52), 1.997 (7.11), 2.327 (0.75), 2.523 (1.72), 2.669 (0.76), 3.735 (9.42), 3.745 (4.95), 3.752 (16.00), 3.770 (7.96), 4.404 (8.55), 4.411 (8.77), 6.958 (1.28), 6.964 (2.69), 6.970 (1.62), 6.981 (2.59), 6.987 (5.17), 6.993 (3.07), 7.004 (1.38), 7.010 (2.63), 7.016 (1.49), 7.460 (1.20), 7.467 (1.49), 7.472 (6.64), 7.478 (7.94), 7.482 (3.17), 7.498 (7.85), 7.504 (6.53), 7.509 (1.39), 7.515 (1.32).

### Intermediate I128

### 2-[2-(3,5-difluorophenyl)-1-oxo-2-azaspiro[4.5]dec-8-yl]-1H-isoindole-1,3(2H)-dione (isomer 1)

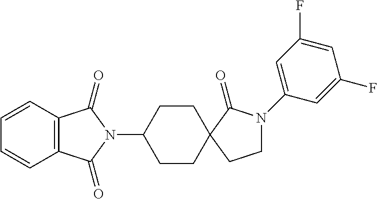 [SMILES: O=C1c2ccccc2C(=O)N1C1CCC2(CC1)CCN(c1cc(F)cc(F)c1)C2=O]

Diisopropyl azodicarboxylate (370 μl, 1.9 mmol) was added drop wise to a mixture of 2-(3,5-difluorophenyl)-8-hydroxy-2-azaspiro[4.5]decan-1-one (isomer 1) (355 mg, 1.26 mmol), phthalimide (279 mg, 1.89 mmol, CAS No 85-41-6) and triphenylphosphine (497 mg, 1.89 mmol) in tetrahydrofuran (15 ml) and the mixture was stirred at room temperature overnight. For work-up, the reaction mixture was concentrated and the residue was purified by flash chromatography (25 g Snap Cartridge, hexanes/ethyl acetate gradient, 0%->50% ethyl acetate) followed by recrystallization from methanol to give the title compound (203 mg) as single isomer, together with unknown impurities.

LC-MS (Method 1): Rₜ=1.40 min; MS (ESIpos): m/z=411.3 [M+H]⁺

¹H-NMR (400 MHz, DMSO-d6) δ[ppm]: 1.172 (0.71), 1.645 (1.93), 1.655 (2.38), 1.677 (1.77), 1.699 (4.64), 1.705 (4.64), 1.730 (3.01), 1.738 (2.67), 1.988 (1.06), 2.116 (3.10), 2.134 (5.42), 2.151 (3.27), 2.219 (1.57), 2.230 (1.87), 2.251 (1.70), 2.262 (1.65), 2.327 (1.01), 2.518 (2.97), 2.523 (2.08), 2.665 (0.71), 2.669 (1.02), 3.821 (3.42), 3.839 (5.73), 3.856 (3.34), 4.034 (0.94), 4.045 (1.42), 6.994 (1.39), 7.000 (0.91), 7.012 (1.42), 7.017 (2.81), 7.023 (1.73), 7.041 (1.34), 7.046 (0.83), 7.499 (3.35), 7.505 (4.00), 7.509 (1.54), 7.525 (3.96), 7.530 (3.34), 7.815 (12.58), 7.823 (2.53), 7.830 (1.68), 7.834 (3.76), 7.841 (4.21), 7.846 (14.71), 7.854 (16.00), 7.860 (5.45), 7.866 (3.96), 7.870 (1.65), 7.877 (2.20).

### Intermediate I129

### 8-amino-2-(3,5-difluorophenyl)-2-azaspiro[4.5]decan-1-one (isomer 1)

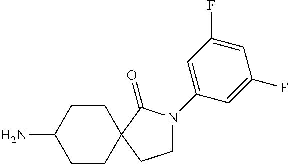 [SMILES: NC1CCC2(CC1)CCN(c1cc(F)cc(F)c1)C2=O]

A mixture of 2-[2-(3,5-difluorophenyl)-1-oxo-2-azaspiro[4.5]dec-8-yl]-1H-isoindole-1,3(2H)-dione (isomer 1) (203 mg, 495 μmol) and hydrazine hydrate (120 μl, 2.5 mmol) in ethanol (4.2 ml) was stirred at 80° C. for 3 h. Upon cooling, the precipitate was filtrated off, washed with ethanol and the filtrate was concentrated to give the title compound (105 mg), which was used in the next step without further purification.

LC-MS (Method 2): Rₜ=1.03 min; MS (ESIpos): m/z=281.2 [M+H]⁺

¹H-NMR (400 MHz, DMSO-d6) δ[ppm]: 1.030 (0.57), 1.048 (1.25), 1.065 (1.80), 1.082 (1.55), 1.092 (3.05), 1.108 (3.48), 1.123 (3.82), 1.141 (3.44), 1.151 (2.12), 1.168 (2.00), 1.507 (13.82), 1.516 (16.00), 1.524 (7.74), 1.533 (11.06), 1.541 (7.10), 1.566 (0.96), 1.575 (0.86), 1.683 (4.85), 1.693 (4.98), 1.705 (2.73), 1.717 (4.60), 1.726 (4.30), 1.766 (1.02), 1.973 (8.33), 1.990 (14.43), 2.008 (8.60), 2.318 (1.09), 2.323 (1.43), 2.327 (1.09), 2.331 (0.64), 2.525 (4.42), 2.535 (2.53), 2.544 (1.50), 2.553 (1.98), 2.563 (1.07), 2.659 (1.14), 2.665 (1.52), 2.669 (1.23), 3.317 (2.96), 3.424 (0.96), 3.442 (0.82), 3.742 (8.63), 3.752 (2.37), 3.761 (14.34), 3.768 (2.64), 3.778 (8.40), 6.961 (1.39), 6.967 (2.89), 6.973 (1.91), 6.984 (2.89), 6.990 (5.64), 6.996 (3.53), 7.008 (1.52), 7.013 (2.84), 7.019 (1.73), 7.450 (0.66), 7.456 (1.46), 7.462 (1.87), 7.467 (7.12), 7.473 (9.01), 7.493 (9.01), 7.499 (7.74), 7.504 (1.96), 7.511 (1.48), 7.518 (0.75), 7.758 (1.41), 7.766 (1.32), 7.773 (1.32), 7.781 (1.57), 8.018 (1.66), 8.026 (1.30), 8.032 (1.30), 8.041 (1.30).

### Intermediate I130

### 8-{[tert-butyl(diphenyl)silyl]oxy}-2-(2-chlorophenyl)-2-azaspiro[4.5]decan-1-one (mixture of cis-/trans-isomers)

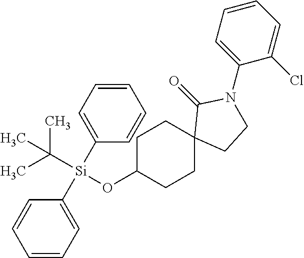 [SMILES: CC(C)(C)[Si](OC1CCC2(CC1)CCN(c1ccccc1Cl)C2=O)(c1ccccc1)c1ccccc1]

Lithium 1,1,1,3,3,3-hexamethyldisilazan-2-ide (42 ml, 1 M solution in tetrahyrofuran, 42 mmol, CAS No 4039-32-1) was added drop wise within 5 minutes to a solution of 2-chloroaniline (2.5 ml, 23 mmol, CAS 95-51-2) in tetrahydrofuran (110 ml) at −78° C. and the mixture was stirred for 60 min at that temperature. A solution of ethyl 4-{[tert-butyl(diphenyl)silyl]oxy}-1-(2-chloroethyl)cyclohexanecarboxylate (mixture of cis/trans isomers) (10.0 g, 21.1 mmol) in tetrahydrofuran (110 ml) was added and the mixture was stirred for 2 h at −78° C. The mixture was warmed to room temperature and stirred for 4 d. For work-up, the reaction was added to a solution of water and sodium bicarbonate and the mixture was extracted with ethyl acetate (3×). The combined organic phases were washed with a saturated sodium chloride solution, dried over sodium sulfate and concentrated under reduced pressure, the residue was purified by flash chromatography (hexane/ethyl acetate gradient, 0%->25% ethyl acetate) to give the title compound in 2 fractions: fraction 1 (6.58 g, single isomer based on ¹H NMR, isomer 1), fraction 2 (1.38 g, single isomer based on ¹H NMR, isomer 2).

### Fraction 1 (Isomer 1):

¹H-NMR (400 MHz, DMSO-d₆): δ [ppm]=7.67-7.55 (m, 5H), 7.52-7.30 (m, 9H), 3.97 (br s, 1H), 3.60 (t, 2H), 2.15-2.07 (m, 2H), 2.04 (t, 2H), 1.76-1.63 (m, 2H), 1.58-1.45 (m, 2H), 1.39-1.27 (m, 2H), 1.05 (s, 9H).

LC-MS (Method 1): Rₜ=1.80 min; MS (ESIpos): m/z=518.3 [M+H]⁺

### Fraction 2 (Isomer 2):

¹H-NMR (400 MHz, DMSO-d6) δ[ppm]=7.68-7.60 (m, 4H), 7.57-7.30 (m, 10H), 3.71-3.62 (m, 1H), 3.60 (t, 2H), 2.09 (t, 2H), 1.83-1.73 (m, 2H), 1.61-1.35 (m, 6H), 1.03 (s, 9H).

LC-MS (Method 1): Rₜ=1.82 min; MS (ESIpos): m/z=518.2 [M+H]⁺

### Intermediate I131

### 2-(2-chlorophenyl)-8-hydroxy-2-azaspiro[4.5]decan-1-one (isomer1)

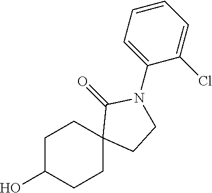 [SMILES: O=C1N(c2ccccc2Cl)CCC12CCC(O)CC2]

A solution of 8-{[tert-butyl(diphenyl)silyl]oxy}-2-(2-chlorophenyl)-2-azaspiro[4.5]decan-1-one (isomer1) (6.58 g, 12.7 mmol) and tetra-butylamonium fluoride (19 ml, 1.0 M, 19 mmol, CAS No 429-41-4) in tetrahydrofuran (100 ml) was stirred overnight at room temperature, then the mixture was heated to reflux and was stirred under reflux for 6 h. For work-up the mixture was concentrated, and the residue was purified by flash chromatography (methylene chloride/ethyl acetate gradient, 0%->100% ethyl acetate), the product containing fractions were concentrated and the resulting precipitate was filtrated off and washed with ethyl acetate to give the title compound (2.87 g).

LC-MS (Method 1): Rₜ=0.95 min; MS (ESIpos): m/z=280.2 [M+H]⁺

¹H-NMR (400 MHz, DMSO-d6) δ[ppm]: 1.266 (1.31), 1.277 (2.48), 1.288 (1.54), 1.299 (1.50), 1.310 (2.68), 1.321 (1.50), 1.517 (1.13), 1.544 (2.15), 1.552 (2.32), 1.560 (1.32), 1.570 (1.50), 1.579 (2.09), 1.587 (1.23), 1.667 (2.31), 1.679 (2.44), 1.691 (1.47), 1.701 (1.59), 1.714 (1.43), 1.924 (1.88), 1.934 (1.98), 1.952 (2.17), 1.957 (2.50), 1.962 (2.31), 1.967 (2.07), 1.985 (1.68), 1.988 (1.48), 1.995 (1.43), 2.023 (4.60), 2.040 (8.41), 2.057 (4.78), 3.584 (5.50), 3.601 (9.02), 3.618 (5.21), 3.736 (1.68), 3.743 (1.66), 4.387 (6.30), 4.395 (6.08), 7.346 (1.34), 7.354 (1.75), 7.360 (1.85), 7.364 (1.31), 7.369 (4.25), 7.376 (1.80), 7.379 (3.50), 7.386 (12.67), 7.390 (16.00), 7.394 (8.00), 7.398 (1.43), 7.402 (2.42), 7.405 (2.71), 7.409 (1.23), 7.538 (0.76), 7.542 (3.23), 7.546 (3.78), 7.549 (2.58), 7.560 (2.60),7.563 (4.00), 7.567 (2.82).

### Intermediate I132

### 2-(2-chlorophenyl)-1-oxo-2-azaspiro[4.5]dec-8-yl methanesulfonate (isomer 1)

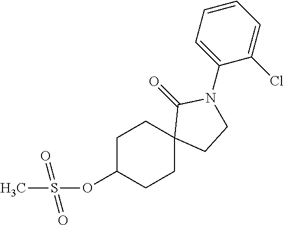 [SMILES: CS(=O)(=O)OC1CCC2(CC1)CCN(c1ccccc1Cl)C2=O]

Triethylamine (1.7 ml) was added to a solution of 2-(2-chlorophenyl)-8-hydroxy-2-azaspiro[4.5]decan-1-one (isomer 1) (2.86 g, 10.2 mmol) in methylene chloride (24 ml) at −20° C., then methanesulfonyl chloride (960 μl, 12 mmol) was added drop wise and the mixture was stirred for 60 min at room temperature. The reaction mixture was diluted with methylene chloride and then washed with sodium bicarbonate and sodium chloride, the organic phase was filtrated through a hydrophobic filter and was then concentrated under reduced pressure to give the title compound (3.68 g).

LC-MS (Method 1): Rₜ=1.03 min; MS (ESIpos): m/z=358.0 [M+H]⁺

¹H-NMR (400 MHz, DMSO-d6) b [ppm]: 2.074 (1.35), 2.091 (2.54), 2.109 (1.41), 3.195 (16.00), 3.331 (10.85), 3.350 (1.28), 3.613 (1.63), 3.631 (2.80), 3.648 (1.56), 7.369 (0.85), 7.381 (1.17), 7.400 (2.48), 7.405 (3.41), 7.408 (4.42), 7.415 (1.22), 7.418 (1.60), 7.553 (1.01), 7.557 (1.49), 7.559 (0.85), 7.571 (0.82), 7.575 (1.27).

### Intermediate I133

### 8-azido-2-(2-chlorophenyl)-2-azaspiro[4.5]decan-1-one (isomer 1)

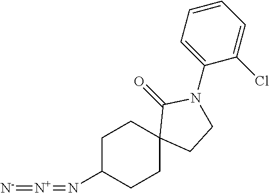 [SMILES: [N-]=[N+]=NC1CCC2(CC1)CCN(c1ccccc1Cl)C2=O]

A suspension of 2-(2-chlorophenyl)-1-oxo-2-azaspiro[4.5]dec-8-yl methanesulfonate (isomer 1) (3.67 g, 10.3 mmol) and sodium azide (867 mg, 13.3 mmol, CAS No 26628-22-8) in N,N-dimethylformamide (32 ml) was stirred at 80° C. for 5.5 h. The mixture was poured into water, stirred for 15 minutes, the resulting precipitate was filtrated off to give the title compound (2.58 g), which was used immediately in the next step without further purification.

LC-MS (Method 1): Rₜ=1.23 min; MS (ESIpos): m/z=305.1 [M+H]⁺

### Intermediate I134

### 8-amino-2-(2-chlorophenyl)-2-azaspiro[4.5]decan-1-one (isomer 1)

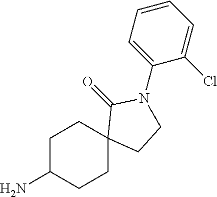 [SMILES: NC1CCC2(CC1)CCN(c1ccccc1Cl)C2=O]

To the solution of 8-azido-2-(2-chlorophenyl)-2-azaspiro[4.5]decan-1-one (isomer 1) (2.58 g, 8.47 mmol) in tetrahydrofuran (29 ml), were added triphenylphosphane (2.66 g, 10.2 mmol) and distilled water (460 μl). The mixture was stirred for 15 h at room temperature and then at 50° C. for 2 h. For work up the mixture was concentrated under reduced pressure and the residue was purified by flash chromatography (25 g Snap cartridge, methylene chloride/methanol gradient, 0%->100% methanol), to give the title compound (1.93 g).

LC-MS (Method 2): Rₜ=0.88 min; MS (ESIpos): m/z=279.2 [M+H]⁺

¹H-NMR (400 MHz, DMSO-d6) δ[ppm]: 1.077 (0.64), 1.098 (1.28), 1.128 (2.06), 1.157 (1.61), 1.180 (0.91), 1.515 (2.39), 1.541 (5.41), 1.549 (6.94), 1.564 (6.62), 1.572 (7.02), 1.703 (2.34), 1.710 (2.38), 1.722 (1.34), 1.733 (2.18), 1.743 (2.04), 2.044 (5.13), 2.052 (0.81), 2.061 (8.77), 2.071 (0.84), 2.078 (5.31), 2.327 (0.53), 2.518 (3.70), 2.523 (1.79), 2.527 (1.36), 2.534 (0.76), 2.544 (1.03), 2.554 (0.48), 2.669 (0.55), 3.590 (5.77), 3.599 (0.92), 3.608 (8.73), 3.617 (0.92), 3.625 (5.57), 7.348 (1.09), 7.355 (1.57), 7.363 (2.48), 7.370 (5.30), 7.376 (3.51), 7.382 (6.60), 7.387 (16.00), 7.392 (7.51), 7.403 (3.80), 7.407 (0.94), 7.417 (0.59), 7.422 (0.75), 7.426 (0.50), 7.542 (5.55), 7.547 (3.46), 7.552 (1.73), 7.557 (1.18), 7.562 (4.24), 7.567 (2.31), 7.571 (0.87).

### Intermediate I135

### phenyl 5-{[2-(2-chlorophenyl)-1-oxo-2-azaspiro[4.5]dec-8-yl]carbamoyl}-1H-imidazole-4-carboxylate (isomer 1)

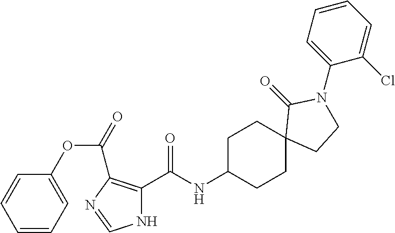 [SMILES: O=C(Oc1ccccc1)c1nc[nH]c1C(=O)NC1CCC2(CC1)CCN(c1ccccc1Cl)C2=O]

To the solution of 8-amino-2-(2-chlorophenyl)-2-azaspiro[4.5]decan-1-one (isomer 1) (1.00 g, 3.59 mmol) in tetrahydrofuran (55 ml), diphenyl 5,10-dioxo-5H,10H-diimidazo[1,5-a:1′,5′-d]pyrazine-1,6-dicarboxylate (922 mg, 2.15 mmol) and triethylamine (600 μl) were added. The mixture was stirred for 15 h at room temperature. The reaction mixture was concentrates and the residue was purified by flash chromatography (40 g Snap cartridge, methylene chloride/methanol gradient, 0%->5% methanol), to give the title compound (1.10 g).

LC-MS (Method 1): Rₜ=1.16 min; MS (ESIpos): m/z=493.1 [M+H]⁺

¹H-NMR (400 MHz, DMSO-d6) δ[ppm]: 1.333 (0.61), 1.349 (0.70), 1.361 (0.81), 1.378 (0.73), 1.619 (2.61), 1.627 (2.93), 1.644 (2.04), 1.908 (0.89), 1.935 (0.83), 2.045 (1.43), 2.062 (2.70), 2.080 (1.48), 3.574 (1.66), 3.591 (2.92), 3.608 (1.60), 3.730 (0.40), 3.739 (0.47), 3.750 (0.43), 3.758 (0.48), 5.760 (16.00), 7.285 (2.51), 7.305 (3.01), 7.333 (1.70), 7.352 (1.20), 7.362 (0.88), 7.370 (1.46), 7.378 (1.47), 7.386 (6.03), 7.400 (1.63), 7.473 (2.25), 7.493 (2.86), 7.513 (1.37), 7.541 (1.06), 7.546 (1.37), 7.557 (0.82), 7.561 (1.43), 7.950 (4.39), 13.597 (0.58).

### Intermediate I136

### 8-{[tert-butyl(diphenyl)silyl]oxy}-2-(2-chloro-4-fluoro-5-methylphenyl)-2-azaspiro[4.5]decan-1-one (mixture of cis-/trans-isomers)

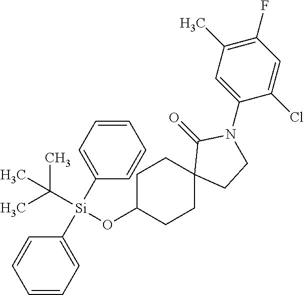 [SMILES: Cc1cc(N2CCC3(CCC(O[Si](c4ccccc4)(c4ccccc4)C(C)(C)C)CC3)C2=O)c(Cl)cc1F]

Lithium 1,1,1,3,3,3-hexamethyldisilazan-2-ide (42 ml, 1 M solution in tetrahyrofuran, 42 mmol, CAS No 4039-32-1) was added drop wise within 5 minutes to a solution of 2-chloro-4-fluoro-5-methylaniline (3.79 g, 23.2 mmol, CAS No 124185-35-9) in tetrahydrofuran (110 ml) at −78° C. The mixture was stirred for 30 min at that temperature. A solution of ethyl 4-{[tert-butyl(diphenyl)silyl]oxy}-1-(2-chloroethyl)cyclohexanecarboxylate (mixture of cis/trans isomers) (10.0 g, 21.1 mmol) in tetrahydrofuran (110 ml) was added and the mixture was stirred for 2 h at −78° C. The mixture was warmed to room temperature and stirred for 4 d. For work-up, the reaction was added to a solution of water and sodium bicarbonate and the mixture was extracted with ethyl acetate (3×). The combined organic phases were washed with sodium chloride, dried over sodium sulfate and concentrated under reduced pressure, the residue was purified by flash chromatography (100 g Snap cartridge, hexane/ethyl acetate gradient, 5%->30% ethyl acetate) the product containing fractions were concentrated and were then purified a second time by flash chromatography (120 g Snap cartridge, hexane/ethyl acetate gradient, 5%->40% ethyl acetate to give the title compound in 2 fractions: fraction 1 (5.85 g, single isomer based on ¹H NMR, isomer 1), fraction 2 (1.21 g, single isomer based on ¹H NMR, isomer 2).

### Fraction 1 (Isomer 1):

¹H-NMR (400 MHz, DMSO-d₆): δ [ppm]=7.65-7.58 (m, 4H), 7.52-7.34 (m, 8H), 3.96 (br s, 1H), 3.56 (t, 2H), 2.23 (d, 3H), 2.09 (td, 2H), 2.04-1.99 (m, 2H), 1.72-1.59 (m, 2H), 1.56-1.45 (m, 2H), 1.31 (m, 2H), 1.04 (s, 9H).

### Fraction 2 (Isomer 2):

¹H-NMR (400 MHz, DMSO-d6) δ[ppm]=7.66-7.59 (m, 4H), 7.50-7.41 (m, 7H), 7.33 (d, 1H), 3.70-3.60 (m, 1H), 3.58-3.52 (m, 2H), 2.20 (d, 3H), 2.07 (t, 2H), 1.77 (br dd, 2H), 1.58-1.33 (m, 6H), 1.02 (s, 9H).

### Intermediate I137

### 2-(2-chloro-4-fluoro-5-methylphenyl)-8-hydroxy-2-azaspiro[4.5]decan-1-one (isomer 1)

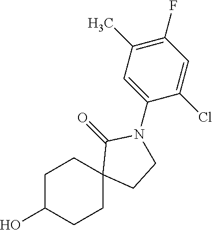 [SMILES: Cc1cc(N2CCC3(CCC(O)CC3)C2=O)c(Cl)cc1F]

A mixture of 8-{[tert-butyl(diphenyl)silyl]oxy}-2-(2-chloro-4-fluoro-5-methylphenyl)-2-azaspiro[4.5]decan-1-one (isomer 1) (5.85 g, 10.6 mmol) and N,N,N-tributylbutan-1-aminium fluoride (16 ml, 1.0 M, 16 mmol, CAS No 429-41-4) in tetrahydrofuran (86 ml), was stirred overnight at 40° C. The reaction was heated to reflux and was stirred for 6 h under reflux. For work-up, the mixture was concentrated under reduced pressure, the residue was purified by flash chromatography (100 g Snap cartridge, methylene chloride/ethyl acetate gradient, 0%->100% ethyl acetate) to give the title compound (3.08 g).

LC-MS (Method 1): Rₜ=1.07 min; MS (ESIpos): m/z=312.2 [M+H]⁺

¹H-NMR (400 MHz, DMSO-d6) δ[ppm]: 0.908 (0.90), 1.251 (1.39), 1.262 (2.68), 1.275 (1.73), 1.283 (1.75), 1.295 (3.03), 1.307 (1.69), 1.503 (0.77), 1.511 (1.22), 1.520 (0.90), 1.537 (2.42), 1.545 (2.58), 1.564 (1.70), 1.572 (2.25), 1.582 (1.24), 1.648 (0.99), 1.659 (2.42), 1.672 (2.60), 1.683 (1.59), 1.693 (1.71), 1.705 (1.54), 1.911 (1.90), 1.921 (2.07), 1.944 (2.71), 1.953 (2.22), 1.972 (1.73), 1.982 (1.46), 1.988 (0.84), 2.007 (4.49), 2.024 (8.49), 2.041 (4.70), 2.215 (15.90), 2.220 (16.00), 2.518 (1.55), 2.523 (1.10), 3.545 (5.40), 3.553 (1.05), 3.563 (8.86), 3.571 (1.08), 3.579 (5.13), 3.731 (1.81), 3.738 (1.81), 4.385 (7.03), 4.392 (7.00), 7.345 (4.32), 7.347 (4.05), 7.364 (4.02), 7.367 (3.93), 7.471 (6.69), 7.495 (6.65).

### Intermediate I138

### 2-(2-chloro-4-fluoro-5-methylphenyl)-1-oxo-2-azaspiro[4.5]dec-8-yl methanesulfonate (isomer 1)

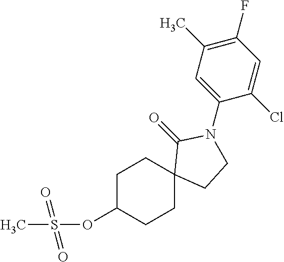 [SMILES: Cc1cc(N2CCC3(CCC(OS(C)(=O)=O)CC3)C2=O)c(Cl)cc1F]

Triethylamine (1.7 ml) was added to a solution of 2-(2-chloro-4-fluoro-5-methylphenyl)-8-hydroxy-2-azaspiro[4.5]decan-1-one (isomer 1) (3.05 g, 9.78 mmol) in methylene chloride (23 ml) at −20° C., then methanesulfonyl chloride (92 0 μl, 12 mmol) was added drop wise and the mixture was stirred for 60 min at room temperature. The reaction mixture was diluted with methylene chloride and then washed with saturated sodium bicarbonate solution and saturated sodium chloride solution, the organic phase was filtrated through a hydrophobic filter and was then concentrated under reduced pressure to give the title compound (3.89 g).

LC-MS (Method 1): Rₜ=1.15 min; MS (ESIpos): m/z=390.1 [M+H]⁺

¹H-NMR (400 MHz, DMSO-d6) δ[ppm]: 0.896 (0.42), 0.914 (1.00), 0.932 (0.49), 1.158 (0.67), 1.175 (1.45), 1.194 (0.73), 1.451 (0.89), 1.461 (0.53), 1.484 (0.82), 1.494 (0.52), 1.784 (0.82), 1.811 (0.58), 1.817 (0.98), 1.840 (1.02), 1.845 (0.99), 1.870 (0.97), 1.907 (0.46), 1.976 (0.70), 1.985 (0.69), 1.996 (0.59), 2.010 (0.64), 2.021 (0.53), 2.057 (1.35), 2.074 (2.55), 2.091 (1.38), 2.219 (5.20), 2.223 (5.27), 2.291 (1.15), 3.193 (16.00), 3.350 (0.94), 3.359 (1.43), 3.575 (1.57), 3.592 (2.72), 3.609 (1.50), 4.854 (0.59), 4.861 (0.72), 4.867 (0.59), 7.369 (1.34), 7.389 (1.33), 7.482 (1.97), 7.505 (1.97).

### Intermediate I139

### 8-azido-2-(2-chloro-4-fluoro-5-methylphenyl)-2-azaspiro[4.5]decan-1-one (isomer 1)

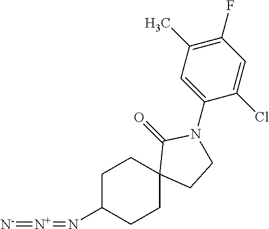 [SMILES: Cc1cc(N2CCC3(CCC(N=[N+]=[N-])CC3)C2=O)c(Cl)cc1F]

A mixture of 2-(2-chloro-4-fluoro-5-methylphenyl)-1-oxo-2-azaspiro[4.5]dec-8-yl methanesulfonate (isomer 1) (3.88 g, 9.95 mmol) and sodium azide (841 mg, 12.9 mmol, CAS No 26628-22-8) in N,N-dimethylformamide (31 ml, 400 mmol) was stirred at 80° C. for 5.5 h. The mixture was poured into water, stirred for 15 minutes, the resulting precipitate was filtrated off to give the title compound (3.23 g), which was used immediately in the next step without further purification.

### Intermediate I140

### 8-amino-2-(2-chloro-4-fluoro-5-methylphenyl)-2-azaspiro[4.5]decan-1-one (isomer 1)

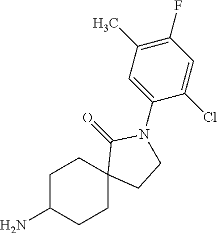 [SMILES: Cc1cc(N2CCC3(CCC(N)CC3)C2=O)c(Cl)cc1F]

To the solution of 8-azido-2-(2-chloro-4-fluoro-5-methylphenyl)-2-azaspiro[4.5]decan-1-one (isomer 1) (3.23 g, 9.59 mmol) in tetrahydrofuran (33 ml, 400 mmol), was added triphenylphosphane (3.02 g, 11.5 mmol) and distilled water (520 μl, 29 mmol). The mixture was stirred for 15 h at room temperature and then for 2 h at 50° C. The mixture was concentrated and the residue was purified by flash chromatography (25 g Snap cartridge, methylene chloride/methanol gradient, 0%->100% methanol), to give the title compound (2.47 g).

LC-MS (Method 2): Rₜ=1.02 min; MS (ESIpos): m/z=311.2 [M+H]⁺

¹H-NMR (400 MHz, DMSO-d6) δ[ppm]: 1.420 (1.60), 1.434 (2.04), 1.450 (1.93), 1.463 (2.30), 1.585 (1.11), 1.610 (2.98), 1.619 (2.98), 1.648 (5.92), 1.891 (2.48), 1.914 (2.24), 2.043 (3.71), 2.060 (7.26), 2.078 (3.98), 2.216 (15.15), 2.220 (16.00), 3.156 (3.78), 3.580 (4.20), 3.597 (7.23), 3.614 (4.07), 5.754 (13.93), 7.346 (4.03), 7.348 (4.01), 7.365 (4.04), 7.367 (3.87), 7.480 (5.98), 7.504 (5.98).

### Intermediate I141

### phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1)

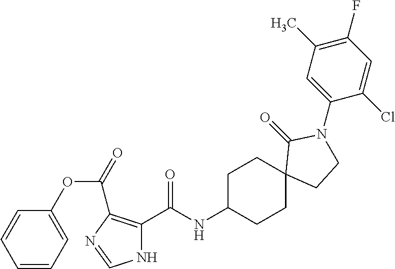 [SMILES: Cc1cc(N2CCC3(CCC(NC(=O)c4[nH]cnc4C(=O)Oc4ccccc4)CC3)C2=O)c(Cl)cc1F]

To the solution of 8-amino-2-(2-chloro-4-fluoro-5-methylphenyl)-2-azaspiro[4.5]decan-1-one (isomer 1) (1.00 g, 3.22 mmol) in tetrahydrofuran (50 ml), diphenyl 5,10-dioxo-5H,10H-diimidazo[1,5-a:1′,5′-d]pyrazine-1,6-dicarboxylate (827 mg, 1.93 mmol) and triethylamine (540 μl) were added. The mixture was stirred for 15 h at room temperature, then concentrated and the residue was purified by flash chromatography (40 g Snap cartridge, methylene chloride/methanol gradient, 0%->5% methanol), to give the title compound (1.39 g).

LC-MS (Method 1): Rₜ=1.25 min; MS (ESIpos): m/z=525.2 [M+H]⁺

¹H-NMR (400 MHz, DMSO-d6) b [ppm]: 1.357 (1.49), 1.604 (4.33), 1.613 (5.25), 1.631 (2.77), 1.639 (2.34), 1.906 (1.55), 1.929 (1.44), 2.028 (2.45), 2.046 (4.76), 2.063 (2.61), 2.207 (11.24), 2.211 (11.05), 2.221 (1.64), 3.534 (2.85), 3.552 (5.08), 3.569 (2.83), 5.759 (16.00), 7.282 (4.05), 7.284 (5.04), 7.287 (2.91), 7.297 (1.78), 7.303 (6.01), 7.306 (4.92), 7.311 (1.85), 7.314 (1.76), 7.328 (1.45), 7.332 (3.37), 7.343 (3.23), 7.348 (2.56), 7.351 (2.37), 7.354 (1.40), 7.363 (3.12), 7.471 (8.17), 7.476 (2.10), 7.485 (1.69), 7.490 (5.66), 7.494 (8.03), 7.506 (1.43), 7.511 (2.91), 7.950 (9.76).

### Intermediate I142

### 8-{[tert-butyl(diphenyl)silyl]oxy}-2-(2-chloro-4,5-difluorophenyl)-2-azaspiro[4.5]decan-1-one (mixture of cis-/trans-isomers)

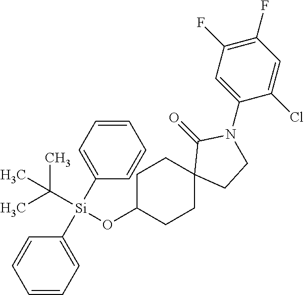 [SMILES: CC(C)(C)[Si](OC1CCC2(CC1)CCN(c1cc(F)c(F)cc1Cl)C2=O)(c1ccccc1)c1ccccc1]

Lithium 1,1,1,3,3,3-hexamethyldisilazan-2-ide (42 ml, 1 M solution in tetrahydrofuran, 42 mmol, CAS No 4039-32-1) was added drop wise within 5 minutes to a solution of 2-chloro-4,5-difluoroaniline (3.88 g, 23.2 mmol) in tetrahydrofuran (110 ml) at −78° C. and the mixture was stirred for 1 h at that temperature. A solution of ethyl 4-{[tert-butyl(diphenyl)silyl]oxy}-1-(2-chloroethyl)cyclohexanecarboxylate (10.0 g, 21.1 mmol) in tetrahydrofuran (110 ml) was added and the mixture was stirred for 2 h at −78° C. The mixture was warmed to room temperature and stirred for 4 d. For work-up, the reaction mixture was added to a mixture of water and sodium bicarbonate solution and the mixture was extracted with ethyl acetate (3×). The combined organic phases were washed with sodium chloride, dried over sodium sulfate and concentrated under reduced pressure, the residue was purified by flash chromatography (340 g Snap cartridge, hexane/ethyl acetate gradient, 5%->25% ethyl acetate). The product containing fractions were concentrated and were purified a second time by flash chromatography (120 g Snap cartridge, hexane/ethyl acetate gradient, 5%->25% ethyl acetate) to give the title compound in 2 fractions: fraction 1 (7.53 g, single isomer based on ¹H NMR, isomer 1, contains impurities from aniline and isomer 2), fraction 2 (1.13 g, isomer 2).

### Fraction 1 (Isomer 1):

¹H-NMR (400 MHz, DMSO-d₆): δ [ppm]=7.67-7.55 (m, 5H), 7.52-7.30 (m, 9H), 3.97 (br s, 1H), 3.60 (t, 2H), 2.15-2.07 (m, 2H), 2.04 (t, 2H), 1.76-1.63 (m, 2H), 1.58-1.45 (m, 2H), 1.39-1.27 (m, 2H), 1.05 (s, 9H).

LC-MS (Method 1): Rₜ=1.80 min; MS (ESIpos): m/z=518.3 [M+H]⁺

### Intermediate I143

### 2-(2-chloro-4,5-difluorophenyl)-8-hydroxy-2-azaspiro[4.5]decan-1-one (isomer 1)

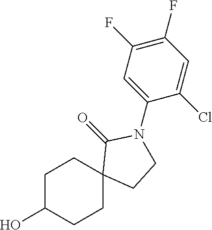 [SMILES: O=C1N(c2cc(F)c(F)cc2Cl)CCC12CCC(O)CC2]

N,N,N-tributylbutan-1-aminium fluoride (38 ml, 1.0 M in THF, 38 mmol, CAS No 429-41-4) was added to a solution of 8-{[tert-butyl(diphenyl)silyl]oxy}-2-(2-chloro-4,5-difluorophenyl)-2-azaspiro [4.5]decan-1-one (isomer 1) (14.2 g, 25.1 mmol) in tetrahydrofuran (200 ml) and the mixture was stirred at 80° C. for 6 h. For work-up, the reaction mixture was diluted with ethyl acetate, washed with sodium bicarbonate and sodium chloride, dried over sodium sulfate and concentrated under reduced pressure. The residue was purified by flash chromatography (120 g Snap cartridge, methylene chloride/ethyl acetate gradient, 0%->10% ethyl acetate) the product containing fractions were concentrated under reduced pressure and the residue was purifies a second time by flash chromatography (120 g Snap cartridge, hexane/ethyl acetate gradient, 20%->100% ethyl acetate to give the title compound (5.31 g).

¹H-NMR (400 MHz, DMSO-d₆): δ [ppm]=7.87 (dd, 1H), 7.70 (dd, 1H), 4.40 (d, 1H), 3.74 (br d, 1H), 3.60 (t, 2H), 2.04 (t, 2H), 1.99-1.90 (m, 2H), 1.69 (dq, 2H), 1.60-1.49 (m, 2H), 1.35-1.24 (m, 2H)

### Intermediate I144

### 2-(2-chloro-4,5-difluorophenyl)-1-oxo-2-azaspiro[4.5]dec-8-yl methanesulfonate (isomer 1)

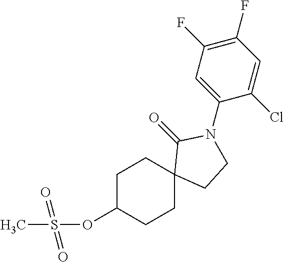 [SMILES: CS(=O)(=O)OC1CCC2(CC1)CCN(c1cc(F)c(F)cc1Cl)C2=O]

Triethylamine (1.6 ml) and methanesulfonyl chloride (890 μl, 11 mmol) were added at 0 to a solution of 2-(2-chloro-4,5-difluorophenyl)-8-hydroxy-2-azaspiro[4.5]decan-1-one (isomer 1) (3.00 g, 9.50 mmol) in methylene chloride (23 ml), the mixture was stirred at 0° C. for 30 minutes. For work-up, the mixture was diluted with methylene chloride, extracted with sodium bicarbonate and with sodium chloride and dried over sodium sulfate, then concentrated under reduced pressure to give the title compound (3.37 g).

LC-MS (Method 1): Rₜ=1.09 min; MS (ESIpos): m/z=394.0 [M+H]⁺

¹H-NMR (400 MHz, DMSO-d₆): δ [ppm]=7.88 (dd, 1H), 7.73 (dd, 1H), 4.92-4.80 (m, 1H), 3.63 (t, 2H), 3.20 (s, 3H), 2.09 (t, 2H), 2.05-1.96 (m, 2H), 1.93-1.74 (m, 4H), 1.55-1.43 (m, 2H).

### Intermediate I145

### 8-azido-2-(2-chloro-4,5-difluorophenyl)-2-azaspiro[4.5]decan-1-one (isomer 1)

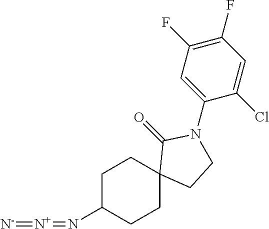 [SMILES: [N-]=[N+]=NC1CCC2(CC1)CCN(c1cc(F)c(F)cc1Cl)C2=O]

Sodium azide (1.23 g, 18.9 mmol, CAS No 26628-22-8) was added to a solution of 2-(2-chloro-4,5-difluorophenyl)-1-oxo-2-azaspiro[4.5]dec-8-yl methanesulfonate (isomer 1) (5.74 g, 14.6 mmol) in dimethylformamide (45 ml), the mixture was stirred at 65° C. for 12 h. For work-up, the mixture was poured in water, the precipitate was filtrated off and washed with water to give the title compound (2.96 g).

LC-MS (Method 1): Rₜ=1.29 min; MS (ESIpos): m/z=341.2 [M+H]⁺

### Intermediate I146

### 8-amino-2-(2-chloro-4,5-difluorophenyl)-2-azaspiro[4.5]decan-1-one (isomer 1)

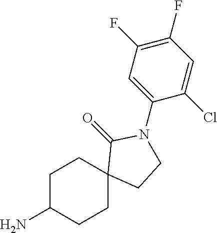 [SMILES: NC1CCC2(CC1)CCN(c1cc(F)c(F)cc1Cl)C2=O]

To a solution of 8-azido-2-(2-chloro-4,5-difluorophenyl)-2-azaspiro[4.5]decan-1-one (isomer 1) (2.96 g, 8.69 mmol) and triphenylphosphane (2.73 g, 10.4 mmol) in tetrahydrofuran (30 ml) was added distilled water (470 μl), the mixture was stirred at room temperature for 24 h.

For work-up the reaction mixture was concentrated under reduced pressure and the residue was purified by flash chromatography (100 g Snap Cartridge, methylene chloride/methanol gradient, 10% methanol) to give the title compounds (963 mg).

LC-MS (Method 1): Rₜ=0.72 min; MS (ESIpos): m/z=315.2 [M+H]⁺

### Intermediate I147

### ethyl 4-{[tert-butyl(diphenyl)silyl]oxy}cyclohexanecarboxylate

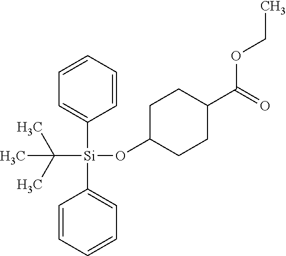 [SMILES: CCOC(=O)C1CCC(O[Si](c2ccccc2)(c2ccccc2)C(C)(C)C)CC1]

Tert-butyl(chloro)diphenylsilane (18 ml, 68 mmol, CAS No 58479-61-1) was added drop wise to a mixture of ethyl 4-hydroxycyclohexanecarboxylate (9.4 ml, 57 mmol, CAS No 17159-80-7), 1H-imidazole (9.68 g, 142 mmol, CAS No 16681-56-4) and N,N-dimethylpyridin-4-amine (348 mg, 2.85 mmol, CAS No 1122-58-3) in dimethylformamide (81 ml), and the mixture was stirred at room temperature for 24 h. For work-up, the mixture was poured into water, extracted with ethyl acetate (3×) and the combined organic phases were washed until pH=7 as reached. The organic phase was dried over sodium sulfate and was concentrated under reduced pressure. The residue was purified by flash chromatography (340 g Snap cartridge, hexane/ethyl acetate gradient, 5%->30% ethyl acetate) to give the title compound (21 g).

### Intermediate I148

### ethyl 4-{[tert-butyl(diphenyl)silyl]oxy}-1-(3-chloropropyl)cyclohexanecarboxylate (mixture of cis/trans isomers)

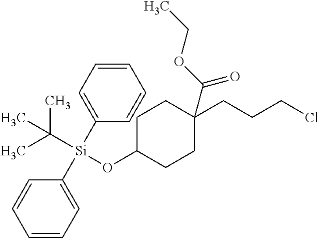 [SMILES: CCOC(=O)C1(CCCCl)CCC(O[Si](c2ccccc2)(c2ccccc2)C(C)(C)C)CC1]

To a solution of ethyl 4-{[tert-butyl(diphenyl)silyl]oxy}cyclohexanecarboxylate (mixture of cis/trans isomers) (38.9 g, 94.7 mmol) in tetrahydrofuran (140 ml) was added lithium di(propan-2-yl)azanide (59 ml, 2.0 M (THF; heptane, ethylbenzene), 120 mmol) at <−70° C., and the mixture was stirred at −78° C. for 30 minutes, then 1-bromo-3-chloropropane (14 ml, 140 mmol, CAS No 109-70-6) was added drop wise. The solution was warmed to room temperature within 2 h. For work-up the mixture was poured in sodium chloride solution, the aqueous phase was extracted with ethyl acetate, the combined organic phases were washed with saturated sodium chloride, filtrated over a hydrophobic filter and concentrated under reduced pressure. The residue was purified by flash chromatography (750 g Snap Cartridge, hexane/ethyl acetate, 0%->10% ethyl acetate) to give the title compound (45 g).

### Intermediate I149

### 9-{[tert-butyl(diphenyl)silyl]oxy}-2-(2-chloro-4-fluoro-5-methylphenyl)-2-azaspiro[5.5]undecan-1-one (isomer 1)

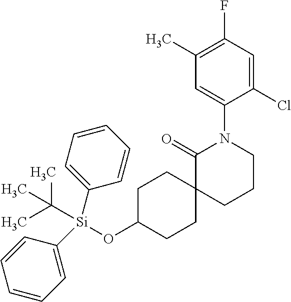 [SMILES: Cc1cc(N2CCCC3(CCC(O[Si](c4ccccc4)(c4ccccc4)C(C)(C)C)CC3)C2=O)c(Cl)cc1F]

Lithium bis(trimethylsilyl)amide (46 ml, 1.0 M in THF, 46 mmol) was added during 5 minutes drop wise to a solution of 2-chloro-4-fluoro-5-methylaniline (4.05 g, 25.4 mmol, CAS No 124185-35-9) in tetrahydrofuran (250 ml) at <−70° C. and the mixture was stirred for 1 h at −78° C. A solution of ethyl-4-{[tert-butyl(diphenyl) silyl]oxy}-1-(3-chloropropyl) cyclohexane carboxylate (mixture of cis/trans isomers) (11.3 g, 23.1 mmol) in tetrahydrofuran (250 ml) was added and the mixture was stirred for 1 h at −78° C. and then at room temperature for 5 d. For work-up, a solution of water and sodium bicarbonate solution was added and the mixture was extracted with ethyl acetate. The combined organic phases were washed with saturated sodium chloride, filtrated through a hydrophobic filter and concentrated. The residue was purified by flash chromatography (340 g Snap Cartridge, hexane/ethyl acetate gradient, 0%->50% ethyl acetate) to give the title compound (6.69 g, based on ¹H NMR predominantly, isomer 1, contains 2-chloro-4-fluoro-5-methylaniline as an impurity).

¹H-NMR (400 MHz, DMSO-d6) δ[ppm]: 1.035 (1.68), 1.043 (16.00), 1.201 (1.36), 1.219 (0.68), 2.016 (2.18), 2.119 (0.88), 2.124 (0.93), 2.248 (3.05), 2.251 (3.04), 7.332 (0.80), 7.352 (0.81), 7.443 (2.42), 7.462 (2.66), 7.469 (2.00), 7.473 (1.17), 7.485 (2.50), 7.508 (1.29), 7.616 (1.72), 7.618 (2.20), 7.620 (2.14), 7.622 (1.84), 7.635 (1.96), 7.638 (1.92), 7.641 (1.10).

### Intermediate I150

### 2-(2-chloro-4-fluoro-5-methylphenyl)-9-hydroxy-2-azaspiro[5.5]undecan-1-one (isomer 1)

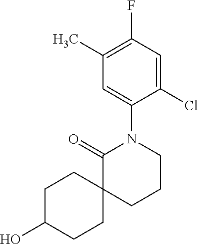 [SMILES: Cc1cc(N2CCCC3(CCC(O)CC3)C2=O)c(Cl)cc1F]

A mixture of 9-{[tert-butyl(diphenyl)silyl]oxy}-2-(2-chloro-4-fluoro-5-methylphenyl)-2-azaspiro[5.5]undecan-1-one (isomer 1) (6.69 g, 8.89 mmol) and N,N,N-tributylbutan-1-aminium fluoride (13 ml, 1.0 M in THF, 13 mmol, CAS No 429-41-4) in tetrahydrofuran (72 ml, 890 mmol), was stirred at 40° C. for 16 hours. The mixture was then heated to reflux and was stirred for 6 h under reflux. For work-up, the mixture was concentrated under reduced pressure and purified by flash chromatography (50 g Snap cartridge, methylene chloride/ethyl acetate gradient, 0%->100% ethyl acetate) to give the title compound (1.88 g).

LC-MS (Method 2): Rₜ=1.16 min; MS (ESIpos): m/z=326.2 [M+H]⁺

### Intermediate I151

### 2-(2-chloro-4-fluoro-5-methylphenyl)-1-oxo-2-azaspiro[5.5]undec-9-yl methanesulfonate (isomer 1)

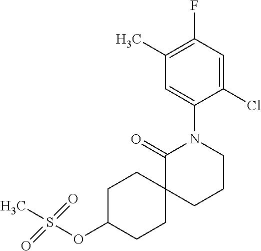 [SMILES: Cc1cc(N2CCCC3(CCC(OS(C)(=O)=O)CC3)C2=O)c(Cl)cc1F]

Methanesulfonyl chloride (480 μl, 6.2 mmol) was added drop wise to a solution of 2-(2-chloro-4-fluoro-5-methylphenyl)-9-hydroxy-2-azaspiro[5.5]undecan-1-one (isomer 1) (1.66 g, 5.09 mmol) in pyridine (10 ml, 120 mmol), the mixture was stirred at room temperature for 90 minutes. For work-up the reaction mixture was poured into water, sodium chloride was added and the solution was stirred for 15 minutes, the resulting precipitate was filtrated, washed with water and then dried do give the title compound (1.87 g) as a crude product which was used in the next step without further purification.

LC-MS (Method 1): Rₜ=1.19 min; MS (ESIpos): m/z=404.1 [M+H]⁺

¹H-NMR (400 MHz, DMSO-d6) δ[ppm]: 1.413 (1.35), 1.514 (1.34), 1.824 (5.81), 1.875 (3.87), 1.888 (4.06), 2.055 (2.42), 2.090 (2.23), 2.214 (12.53), 3.162 (16.00), 3.521 (1.51), 4.785 (2.10), 7.324 (2.60), 7.343 (2.65), 7.443 (2.67), 7.467 (2.67).

### Intermediate I152

### 9-azido-2-(2-chloro-4-fluoro-5-methylphenyl)-2-azaspiro[5.5]undecan-1-one (isomer 1)

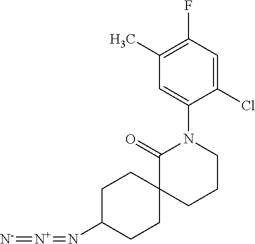 [SMILES: Cc1cc(N2CCCC3(CCC(N=[N+]=[N-])CC3)C2=O)c(Cl)cc1F]

Sodium azide (301 mg, 4.63 mmol) was added to a solution of 2-(2-chloro-4-fluoro-5-methylphenyl)-1-oxo-2-azaspiro[5.5]undec-9-yl methanesulfonate (isomer 1) (1.87 g, 4.63 mmol) in dimethylformamide (14 ml), the mixture was stirred at 70° C. for 2.5 h, then another 0.15 eq of sodium azide (45 mg, 0.69 mmol) were added and the solution was stirred at 70° C. for 1 h. For work-up the mixture was poured into a solution of water and phosphate buffer pH8, the water was decanted and the residue was diluted with tetrahydrofuran to give the title compound as a solution in THF, which was used in the next step without further purification.

### Intermediate I153

### 9-amino-2-(2-chloro-4-fluoro-5-methylphenyl)-2-azaspiro[5.5]undecan-1-one (isomer 1)

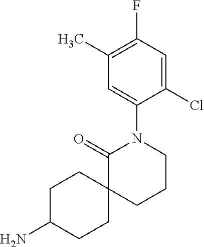 [SMILES: Cc1cc(N2CCCC3(CCC(N)CC3)C2=O)c(Cl)cc1F]

Triphenylphosphane (1.58 g, 6.02 mmol) was added to the crude solution of 9-azido-2-(2-chloro-4-fluoro-5-methylphenyl)-2-azaspiro[5.5]undecan-1-one (isomer 1) (1.62 g, 4.63 mmol) in tetrahydrofuran (17 ml), the mixture was stirred for 16 hours at room temperature. The reaction mixture was concentrated under reduced pressure and the residue was purified by flash chromatography (25 g Snap cartridge, methylene chloride/ethanol gradient, 0%->100% ethanol) to give the title compound (860 mg).

¹H-NMR (400 MHz, DMSO-d6) δ[ppm]: 1.035 (0.85), 1.052 (1.72), 1.070 (0.87), 1.250 (0.82), 1.277 (2.12), 1.311 (2.22), 1.338 (0.99), 1.580 (1.24), 1.613 (1.61), 1.658 (2.70), 1.698 (2.96), 1.729 (1.46), 1.740 (1.32), 1.764 (1.74), 1.774 (1.50), 1.798 (1.99), 1.808 (2.19), 1.833 (3.31), 1.841 (3.63), 1.854 (5.19), 1.864 (4.53), 1.876 (5.47), 1.888 (3.23), 2.205 (15.98), 2.209 (16.00), 2.669 (0.92), 2.678 (1.03), 2.696 (0.99), 2.706 (1.67), 2.735 (0.81), 3.312 (1.58), 3.325 (1.81), 3.339 (2.17), 3.428 (1.04), 3.445 (1.03), 3.472 (1.24), 3.485 (1.93), 3.500 (1.68), 3.514 (1.34), 7.294 (4.01), 7.314 (4.07), 7.437 (5.72), 7.460 (5.69).

### Intermediate I154

### phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1)

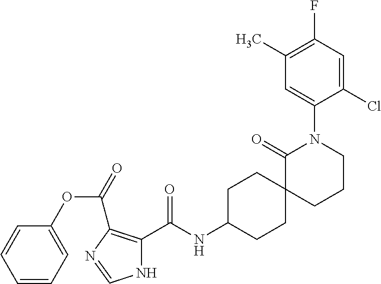 [SMILES: Cc1cc(N2CCCC3(CCC(NC(=O)c4[nH]cnc4C(=O)Oc4ccccc4)CC3)C2=O)c(Cl)cc1F]

Triethylamine (350 μl) was added to a mixture of 9-amino-2-(2-chloro-4-fluoro-5-methylphenyl)-2-azaspiro[5.5]undecan-1-one (isomer 1) (680 mg, 2.09 mmol) and diphenyl 5,10-dioxo-5H,10H-diimidazo[1,5-a:1′,5′-d]pyrazine-1,6-dicarboxylate (538 mg, 1.26 mmol) in tetrahydrofuran (32 ml), the mixture was stirred at room temperature for 15 h. The precipitate was filtrated off. The mother liquor was concentrated under reduced pressure to give the title compound (1.30 g) as crude material which was used in the next steps without further purification.

LC-MS (Method 2): Rₜ=1.13 min; MS (ESIpos): m/z=539.2 [M+H]⁺

### Intermediate I155

### 9-{[tert-butyl(diphenyl)silyl]oxy}-2-(2-chloro-4,5-difluorophenyl)-2-azaspiro[5.5]undecan-1-one (isomer 1)

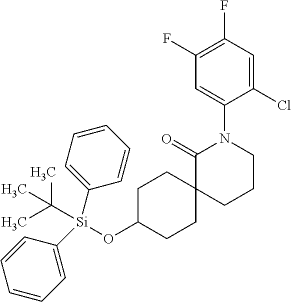 [SMILES: CC(C)(C)[Si](OC1CCC2(CCCN(c3cc(F)c(F)cc3Cl)C2=O)CC1)(c1ccccc1)c1ccccc1]

Lithium bis(trimethylsilyl)amide (46 ml, 1.0 M, 46 mmol) was added during 5 minutes drop wise to a solution of 2-chloro-4,5-difluoroaniline (4.15 g, 25.4 mmol, CAS No 2613-32-3) in tetrahydrofuran (250 ml) at <−70° C. and the mixture was stirred for 1 h at −78° C. A solution of ethyl-4-{[tert-butyl(diphenyl)silyl]oxy}-1-(3-chloropropyl) cyclohexanecarboxylate (mixture of cis/trans isomers) (11.3 g, 23.1 mmol) in tetrahydrofuran (250 ml) was added and the mixture was stirred for 1 h at −78° C. and then at room temperature for 5 d. For work-up, a solution of water and sodium bicarbonate was added and the mixture was extracted with ethyl acetate. The combined organic phases were washed with saturated sodium chloride, filtrated through a hydrophobic filter and concentrated. The residue was purified by flash chromatography (340 g Snap Cartridge, hexane/ethyl acetate gradient, 0%->20% ethyl acetate) to give the title compound (7.68 g, based on ¹H NMR predominantly, isomer 1, contains 2-chloro-4,5 difluoroaniline as an impurity).

¹H-NMR (400 MHz, DMSO-d6) δ[ppm]: 0.980 (1.18), 1.014 (16.00), 1.154 (0.74), 1.172 (1.60), 1.190 (0.81), 1.987 (2.73), 7.398 (0.81), 7.414 (2.57), 7.432 (2.82), 7.440 (2.06), 7.444 (1.32), 7.457 (1.39), 7.587 (2.26), 7.591 (2.21), 7.606 (2.18).

### Intermediate I156

### 2-(2-chloro-4,5-difluorophenyl)-9-hydroxy-2-azaspiro[5.5]undecan-1-one (isomer 1)

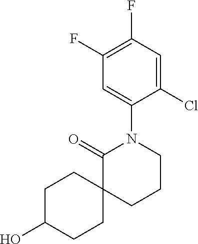 [SMILES: O=C1N(c2cc(F)c(F)cc2Cl)CCCC12CCC(O)CC2]

A mixture of 9-{[tert-butyl(diphenyl)silyl]oxy}-2-(2-chloro-4,5-difluorophenyl)-2-azaspiro[5.5]undecan-1-one (isomer 1) (6.69 g, 8.24 mmol) and N,N,N-tributylbutan-1-aminium fluoride (12 ml, 1.0 M in THF, 12 mmol) in tetrahydrofuran (67 ml), was stirred overnight at 40° C. The mixture was heated to reflux and was stirred for 6 h under reflux. For work-up, the mixture was concentrated under reduced pressure and the residue was purified by flash chromatography (50 g Snap cartridge, methylene chloride/ethyl acetate gradient, 0%->100% ethyl acetate) to give the title compound (1.45 g).

LC-MS (Method 2): Rₜ=1.09 min; MS (ESIpos): m/z=330.1 [M+H]⁺

### Intermediate I157

### 2-(2-chloro-4,5-difluorophenyl)-1-oxo-2-azaspiro[5.5]undec-9-yl methanesulfonate (isomer 1)

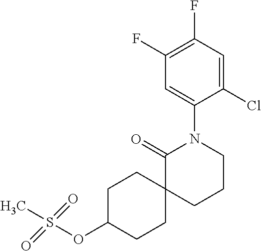 [SMILES: CS(=O)(=O)OC1CCC2(CCCN(c3cc(F)c(F)cc3Cl)C2=O)CC1]

Methanesulfonyl chloride (410 μl, 5.3 mmol) was added drop wise to a solution of 2-(2-chloro-4,5-difluorophenyl)-9-hydroxy-2-azaspiro[5.5]undecan-1-one (isomer 1) (1.44 g, 4.37 mmol) in pyridine (8.6 ml, 110 mmol), then stirred at room temperature for 90 minutes. For work-up the reaction mixture was poured into water, sodium chloride was added, the solution was stirred for 15 min, the precipitate was filtrated off, washed with water and then dried do give the title compound (1.87 g), which was used in the following steps without further purification.

LC-MS (Method 1): Rₜ=1.15 min; MS (ESIpos): m/z=408.1 [M+H]⁺

### Intermediate I158

### 9-azido-2-(2-chloro-4,5-difluorophenyl)-2-azaspiro[5.5]undecan-1-one (isomer 1)

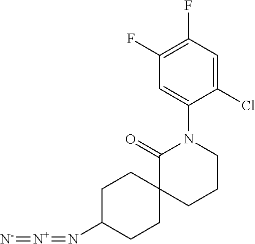 [SMILES: [N-]=[N+]=NC1CCC2(CCCN(c3cc(F)c(F)cc3Cl)C2=O)CC1]

Sodium azide (239 mg, 3.68 mmol) was added to a solution of 2-(2-chloro-4,5-difluorophenyl)-1-oxo-2-azaspiro[5.5]undec-9-yl methanesulfonate (isomer 1) (1.50 g, 3.68 mmol) in dimethylformamide (11 ml), stirred at 70° C. for 3 h, then another 0.15 eq of sodium azide (39 mg, 0.55 mmol) were added. After stirring for additional 3 h at 70° C. the solution was cooled to room temperature and stirred for 16 h. For work-up the mixture was poured into a solution of water and phosphate buffer pH8, the water was decanted, the residue diluted with tetrahydrofuran to give the title compound as a solution in THF, which was used in the next step without further purification.

LC-MS (Method 2): Rₜ=1.36 min; MS (ESIpos): m/z=355.1 [M+H]⁺

### Intermediate I159

### 9-amino-2-(2-chloro-4,5-difluorophenyl)-2-azaspiro[5.5]undecan-1-one (isomer 1)

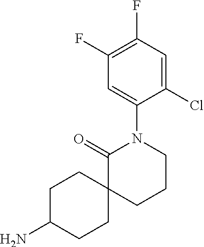 [SMILES: NC1CCC2(CCCN(c3cc(F)c(F)cc3Cl)C2=O)CC1]

Triphenylphosphane (1.25 g, 4.78 mmol) was added to the crude solution of 9-azido-2-(2-chloro-4,5-difluorophenyl)-2-azaspiro[5.5]undecan-1-one (isomer 1) (1.30 g, 3.68 mmol) in tetrahydrofuran (13 ml), the mixture was stirred for 16 hours at room temperature. For work up the reaction mixture was concentrated under reduced pressure and the residue was purified by flash chromatography (25 g Snap cartridge, methylene chloride/methanol gradient, 0%->100% methanol) to give the title compound (780 mg).

### Intermediate I160

### phenyl 5-{[2-(2-chloro-4,5-difluorophenyl)-1-oxo-2-azaspiro[5.5]undec-9-yl]carbamoyl}-1H-imidazole-4-carboxylate (isomer 1)

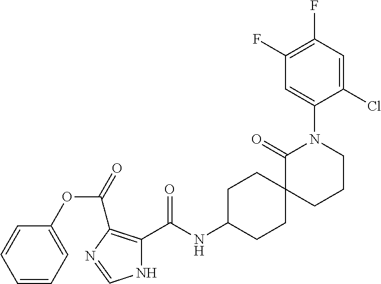 [SMILES: O=C(Oc1ccccc1)c1nc[nH]c1C(=O)NC1CCC2(CCCN(c3cc(F)c(F)cc3Cl)C2=O)CC1]

Triethylamine (280 μl) was added to a mixture of 9-amino-2-(2-chloro-4,5-difluorophenyl)-2-azaspiro[5.5]undecan-1-one (isomer 1) (560 mg, 1.70 mmol) and diphenyl 5,10-dioxo-5H,10H-diimidazo[1,5-a:1′,5′-d]pyrazine-1,6-dicarboxylate (438 mg, 1.02 mmol) in tetrahydrofuran (26 ml), the mixture was stirred at room temperature for 18 h. The precipitate was filtrated off, the mother liquor was concentrated under reduced pressure to give the title compound (1.08 g, 60% purity), which was used in the next step without further purification.

LC-MS (Method 1): Rₜ=1.25 min; MS (ESIpos): m/z=543.2 [M+H]⁺

### Intermediate I161

### 2-(4-chloropyridin-3-yl)-8-hydroxy-2-azaspiro[4.5]decan-1-one (isomer 1)

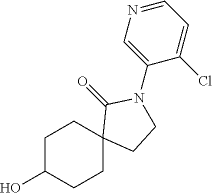 [SMILES: O=C1N(c2cnccc2Cl)CCC12CCC(O)CC2]

A mixture of ethyl 4-hydroxy-1-(2-methoxyethyl)cyclohexanecarboxylate (mixture of cis-/trans-isomers) (450 mg, 1.95 mmol), 4-chloropyridin-3-amine (377 mg, 2.93 mmol, CAS 20511-15-3) and dimethylaluminium chloride (3.9 ml, 1.0 M, 3.9 mmol) in dioxane (8.3 ml), was stirred under argon atmosphere for 16 h at 100° C. Upon cooling, the mixture was poured into ice water, extracted with ethyl acetate (3×) and the combined organic phases were washed with brine, filtrated through a silicone filter and concentrated under reduced pressure. The residue was purified by flash chromatography (25 g Snap cartridge, ethyl acetate/methanol gradient, 0%->10% methanol) to give the title compound (222 mg) as single isomer (isomer 1).

¹H-NMR (500 MHz, DMSO-d₆): δ [ppm]=8.59 (s, 1H), 8.50 (d, 1H), 7.68 (d, 1H), 4.40 (d, 1H), 3.77-3.70 (m, 1H), 3.67 (t, 2H), 2.07 (t, 2H), 2.00-1.92 (m, 2H), 1.73-1.65 (m, 2H), 1.60-1.52 (m, 2H), 1.35-1.27 (m, 2H)

### Intermediate I162

### 2-[2-(4-chloropyrid in-3-yl)-1-oxo-2-azaspiro[4.5]dec-8-yl]-1H-isoindole-1,3(2H)-dione (isomer 1)

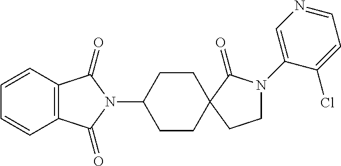 [SMILES: O=C1c2ccccc2C(=O)N1C1CCC2(CC1)CCN(c1cnccc1Cl)C2=O]

Diisopropyl azodicarboxylate (400 μl, 2.0 mmol) was added drop wise to a mixture of 2-(4-chloropyridin-3-yl)-8-hydroxy-2-azaspiro[4.5]decan-1-one (isomer 1) (380 mg, 1.35 mmol), phthalimide (299 mg, 2.03 mmol) and triphenylphosphine (533 mg, 2.03 mmol) in tetrahydrofuran (16 ml) and the mixture was stirred at room temperature for 12 h. For work-up, the reaction mixture was concentrated and the residue was purified by flash chromatography (Snap cartridge, hexanes/ethyl acetate-gradient, 0%->20% ethyl acetate) to provide the title compound as single isomer, together with triphenylphosphine oxide. The crude product was purified by preparative HPLC [Instrument: Waters Autopurificationsystem; column: Waters XBrigde C18 5μ 100×30 mm; eluent A: water+0.2 vol-% trifluoroacetic acid (99%); eluent B: acetonitrile; gradient: 0.00-0.50 min 30% B (25->70 ml/min), 0.51-5.50 min 30-45% B (70 ml/min), DAD scan: 210-400 nm] to give the title compound (249 mg).

LC-MS (Method 2): Rₜ=1.10 min; MS (ESIpos): m/z=410.4 [M+H]⁺

¹H-NMR (400 MHz, DMSO-d6) δ[ppm]: 1.635 (0.48), 1.636 (0.60), 1.648 (1.13), 1.672 (2.22), 1.682 (2.42), 1.705 (1.37), 1.713 (2.68), 1.733 (2.12), 1.758 (5.28), 1.768 (3.65), 1.793 (1.69), 2.216 (3.45), 2.234 (6.88), 2.251 (4.71), 2.264 (2.15), 2.285 (1.88), 2.296 (1.57), 2.318 (0.96), 2.323 (1.09), 2.327 (1.33), 2.331 (1.16), 2.337 (0.50), 2.523 (2.12), 2.540 (0.77), 2.665 (0.73), 2.669 (0.99), 2.674 (0.70), 3.726 (4.06), 3.744 (6.93), 3.761 (3.94), 3.986 (4.12), 4.015 (4.87), 4.024 (5.02), 4.034 (4.46), 4.045 (4.37), 4.054 (4.63), 4.065 (3.55), 4.076 (2.80), 4.085 (2.77), 4.095 (2.15), 7.702 (6.06), 7.716 (6.34), 7.826 (1.81), 7.832 (1.28), 7.837 (3.47), 7.843 (3.82), 7.849 (14.72), 7.856 (16.00), 7.861 (4.88), 7.867 (3.55), 7.872 (1.33), 7.878 (1.76), 8.517 (7.45), 8.530 (7.15), 8.616 (11.27).

### Intermediate I163

### 8-amino-2-(4-chloropyridin-3-yl)-2-azaspiro[4.5]decan-1-one (isomer 1)

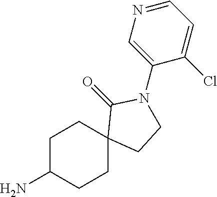 [SMILES: NC1CCC2(CC1)CCN(c1cnccc1Cl)C2=O]

A mixture of 2-[2-(4-chloropyridin-3-yl)-1-oxo-2-azaspiro[4.5]dec-8-yl]-1H-isoindole-1,3(2H)-dione (isomer 1) (240 mg, 586 μmol) and hydrazine hydrate (150 μl, 2.9 mmol) in ethanol (5.0 ml) was stirred at 80° C. for 3 h. Upon cooling, the precipitate was stirred with dichloromethane, the solid was filtrated off and the filtrate was washed with water, filtrated through a silicone filter and concentrated to give the title compound (67.0 mg) which was used without further purification.

LC-MS (Method 2): Rₜ=0.70 min; MS (ESIneg): m/z=480 [M+H]⁻

### Intermediate I164

### ethyl 4-{[tert-butyl(diphenyl)silyl]oxy}-1-(2-chloroethyl)cyclohexanecarboxylate (mixture of cis/trans isomers)

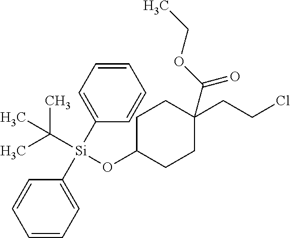 [SMILES: CCOC(=O)C1(CCCl)CCC(O[Si](c2ccccc2)(c2ccccc2)C(C)(C)C)CC1]

Lithium di(propan-2-yl)azanide (98 ml, 2.0 M, 200 mmol, CAS No 4111-54-0) was added drop wise to a solution of ethyl 4-{[tert-butyl(diphenyl)silyl]oxy}cyclohexanecarboxylate (64.2 g, 156 mmol) in tetrahydrofuran (230 ml) at −78° C. to −700, then the mixture was stirred 30 minutes at −78° C. 1-bromo-2-chloroethane (20 ml, 23 0 mmol, CAS No 107-04-0) was added drop wise to the reaction and the mixture was warmed to room temperature within 2 h, then stirred at room temperature for 1.5 h. For work-up, water was added and the mixture was extracted with ethyl acetate. The combined organic phases were washed with half saturated sodium chloride solution (2×) and with saturated sodium chloride solution. The organic phase was filtrated over a hydrophobic filter and concentrated. The residue was purified by flash chromatography (750 g Snap Cartridge, hexane/ethyl acetate gradient, 0%->20% ethyl acetate) to give the title compound (68.51 g).

### Intermediate I165

### 8-{[tert-butyl(diphenyl)silyl]oxy}-2-(3-chlorophenyl)-2-azaspiro[4.5]decan-1-one (mixture of cis/trans isomers)

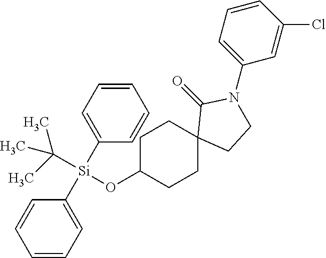 [SMILES: CC(C)(C)[Si](OC1CCC2(CC1)CCN(c1cccc(Cl)c1)C2=O)(c1ccccc1)c1ccccc1]

Bis-(trimethylsilyl)-lithiumamid lithium 1,1,1,3,3,3-hexamethyldisilazan-2-ide (42 ml, 1.0 M in THF, 42 mmol) was added over a period of 5 minutes drop wise to a solution of 3-chloroaniline (2.5 ml, 23 mmol, CAS No 108-42-9) in tetrahydrofuran (110 ml) at −78° C. and the mixture was stirred for 1 h at −78° C. A solution of ethyl 4-{[tert-butyl(diphenyl)silyl]oxy}-1-(2-chloroethyl)cyclohexanecarboxylate (10.0 g, 21.1 mmol) in tetrahydrofuran (110 ml) was added and the mixture was stirred for 2 h at −78° C. and then at room temperature for 4 d. For work-up, a solution of water and sodium bicarbonate was added and the mixture was extracted with ethyl acetate (3×). The combined organic phases were washed with sodium chloride, dried over sodium sulfate, and were concentrated under reduced pressure. The residue was purified by flash chromatography (100 g Snap Cartridge, hexane/ethyl acetate gradient, 7%->60% ethyl acetate) the product containing fractions were combined and were concentrated under reduced pressure. The residue was purified a second time by flash chromatography (120 g Snap Cartridge, hexane/ethyl acetate gradient, 5%->30% ethyl acetate) to give the title compound in 2 fractions: fraction 1 (6.41 g, single isomer based on ¹H NMR, isomer 1), fraction 2 (1.58 g, isomer 2).

### Fraction 1 (Isomer 1):

¹H-NMR (400 MHz, DMSO-d₆): δ [ppm]=7.94 (t, 1H), 7.66-7.55 (m, 5H), 7.52-7.37 (m, 7H), 7.20 (ddd, 1H), 3.97 (br s, 1H), 3.76 (t, 2H), 2.15-2.04 (m, 2H), 1.97 (t, 2H), 1.70-1.59 (m, 2H), 1.54-1.42 (m, 2H), 1.32-1.26 (m, 2H), 1.06 (s, 9H).

### Intermediate I166

### 2-(3-chlorophenyl)-8-hydroxy-2-azaspiro[4.5]decan-1-one (isomer 1)

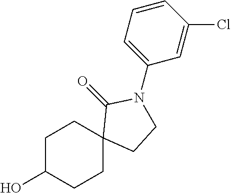 [SMILES: O=C1N(c2cccc(Cl)c2)CCC12CCC(O)CC2]

N,N,N-tributylbutan-1-aminium fluoride (25 ml, 1.0 M in THF, 25 mmol) was added at room temperature to a solution of 8-{[tert-butyl(diphenyl)silyl]oxy}-2-(3-chlorophenyl)-2-azaspiro[4.5]decan-1-one (isomer 1) (6.41 g, 12.4 mmol) in tetrahydrofuran (120 ml), the mixture was stirred at 80° C. for 5 h. For work-up, the mixture was poured into water, extracted with ethyl acetate (3×), the combined organic phases were washed with diluted sodium bicarbonate and with brine. The organic phase was filtrated trough a silicone filter and concentrated under reduced pressure. The residue was purified by flash chromatography (120 g Snap Cartridge, hexane/ethyl acetate gradient, 20%->100% ethyl acetate) to give the title compound (3.1 g).

¹H-NMR (400 MHz, DMSO-d₆): δ [ppm]=7.90 (t, 1H), 7.58 (ddd, 1H), 7.40 (t, 1H), 7.19 (ddd, 1H), 4.41 (d, 1H), 3.81-3.69 (m, 3H), 3.41-3.33 (m, 1H), 3.36-3.28 (m, 1H), 2.02-1.89 (m, 4H), 1.72-1.62 (m, 2H), 1.58-1.49 (m, 2H), 1.26 (dt, 2H).

### Intermediate I167

### 2-(3-chlorophenyl)-1-oxo-2-azaspiro[4.5]dec-8-yl methanesulfonate (isomer 1)

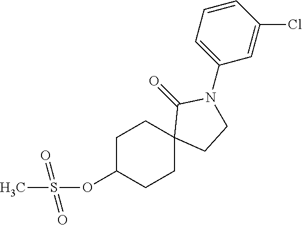 [SMILES: CS(=O)(=O)OC1CCC2(CC1)CCN(c1cccc(Cl)c1)C2=O]

Triethylamine (1.9 ml) and methanesulfonyl chloride (1.0 ml, 13 mmol) were added at 0 to a solution of 2-(3-chlorophenyl)-8-hydroxy-2-azaspiro[4.5]decan-1-one (isomer 1) (3.10 g, 11.1 mmol) in methylene chloride (26 ml), the mixture was stirred at 0° C. for 30 minutes. For work-up, the mixture was diluted with methylene chloride, and the organic phase was washed with sodium bicarbonate and with sodium chloride. The organic phase was dried over sodium sulfate and concentrated under reduced pressure to give the title compound (3.68 g), which was used in the next step without further purification.

¹H-NMR (400 MHz, DMSO-d₆): δ [ppm]=7.89 (t, 1H), 7.62-7.52 (m, 1H), 7.41 (t, 1H), 7.20 (ddd, 1H), 4.92-4.83 (m, 1H), 3.80 (t, 2H), 3.21 (s, 3H), 2.07-1.94 (m, 4H), 1.92-1.74 (m, 4H), 1.51-1.42 (m, 2H).

### Intermediate I168

### 8-azido-2-(3-chlorophenyl)-2-azaspiro[4.5]decan-1-one (isomer 1)

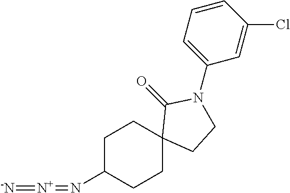

Sodium azide (869 mg, 13.4 mmol) was added to a solution of 2-(3-chlorophenyl)-1-oxo-2-azaspiro[4.5]dec-8-yl methanesulfonate (isomer 1) (3.68 g, 10.3 mmol) in dimethylformamide (32 ml), the reaction was stirred at 65° C. for 10 h. For work-up, the mixture has been poured into water, the precipitate was filtrated off and washed with water to give the title compound (3.22 g), which was used immediately in the next step without further purification.

### Intermediate I169

### 8-amino-2-(3-chlorophenyl)-2-azaspiro[4.5]decan-1-one (isomer 1)

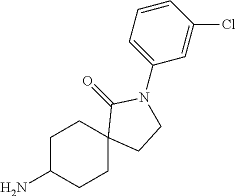 [SMILES: NC1CCC2(CC1)CCN(c1cccc(Cl)c1)C2=O]

To a solution of 8-azido-2-(3-chlorophenyl)-2-azaspiro[4.5]decan-1-one (isomer 1) (3.22 g, 10.6 mmol) and triphenylphosphane (3.33 g, 12.7 mmol) in tetrahydrofuran (36 ml) distilled water (570 μl) was added, then stirred at room temperature for 48 h. For work-up the reaction mixture concentrated under reduced pressure and the residue was purified by flash chromatography (25 g Snap Cartridge, dichloromethane/methanol, 10%->100% methanol) to give the title compound (2.1 g).

### Intermediate I170

### phenyl 5-{[2-(3-chlorophenyl)-1-oxo-2-azaspiro[4.5]dec-8-yl]carbamoyl}-1H-imidazole-4-carboxylate (isomer 1)

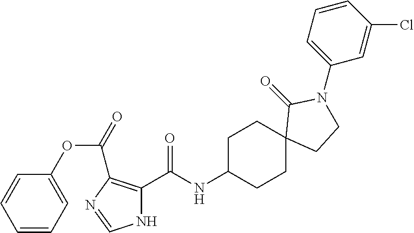 [SMILES: O=C(Oc1ccccc1)c1nc[nH]c1C(=O)NC1CCC2(CC1)CCN(c1cccc(Cl)c1)C2=O]

To the solution of diphenyl 5,10-dioxo-5H,10H-diimidazo[1,5-a:1′,5′-d]pyrazine-1,6-dicarboxylate (isomer 1) (922 mg, 2.15 mmol) in THF (27.5 ml) was added triethylamine (600 μl, 4.3 mmol) and the mixture was stirred at room temperature. A solution of 8-amino-2-(3-chlorophenyl)-2-azaspiro[4.5]decan-1-one (1.00 g, 3.59 mmol) (isomer 1) in THF (27.5 ml) was added and the mixture was stirred for 6 h at room temperature. The reaction mixture was concentrates and the residue was purified by flash chromatography (methylene chloride/methanol gradient, 0%->10% methanol), to give the title compound (1.8 g).

¹H-NMR (400 MHz, DMSO-d6) δ[ppm]: 1.172 (0.43), 1.295 (1.16), 1.316 (3.01), 1.326 (3.26), 1.346 (3.65), 1.356 (3.57), 1.376 (2.00), 1.388 (1.78), 1.569 (3.24), 1.601 (11.47), 1.639 (5.13), 1.665 (1.53), 1.800 (0.64), 1.910 (3.89), 1.934 (3.78), 1.942 (3.74), 1.981 (5.80), 1.999 (9.82), 2.016 (5.37), 2.087 (0.80), 2.322 (0.77), 2.327 (1.09), 2.331 (0.84), 2.518 (4.12), 2.523 (2.69), 2.664 (0.75), 2.669 (1.05), 2.674 (0.73), 3.738 (7.84), 3.755 (13.01), 3.772 (7.75), 5.758 (16.00), 7.170 (5.00), 7.172 (5.69), 7.175 (5.54), 7.177 (5.18), 7.190 (6.53), 7.192 (6.55), 7.195 (6.77), 7.197 (6.21), 7.286 (10.63), 7.305 (13.08), 7.317 (3.42), 7.336 (6.34), 7.354 (3.76), 7.371 (7.19), 7.391 (12.80), 7.411 (7.26), 7.429 (0.67), 7.474 (9.04), 7.493 (11.34), 7.513 (5.28), 7.550 (5.50), 7.553 (5.45), 7.571 (4.38), 7.574 (4.92), 7.596 (0.64), 7.826 (0.54), 7.884 (6.92), 7.889 (11.00), 7.893 (5.78), 7.913 (0.58), 7.922 (0.56), 7.942 (14.35), 7.986 (0.82), 8.146 (0.75), 8.346 (0.41), 9.322 (0.47), 9.399 (4.62), 9.417 (4.47), 13.591 (5.91).

### Intermediate I171

### 5-(morpholin-4-yl)pyrazolo[1,5-a]pyrimidine-3-carboxylic acid

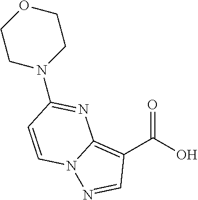 [SMILES: O=C(O)c1cnn2ccc(N3CCOCC3)nc12]

Sodium hydroxide (3.5 ml, 2 M aqueous solution, 7.0 mmol) was added to a solution of ethyl 5-(morpholin-4-yl)pyrazolo[1,5-a]pyrimidine-3-carboxylate (775 mg, 2.80 mmol) in tetrahydrofuran (19 ml) and methanol (5.6 ml), then stirred at 70° C. for 3 h. For work-up, the mixture was concentrated, then poured into water and acidified with citric acid. The precipitate formed was collected by filtration and dried at 50° C. to give the title compound (635 mg).

LC-MS (Method 1): Rₜ=0.57 min; MS (ESIpos): m/z=249 [M+H]⁺

¹H-NMR (500 MHz, DMSO-d6) δ[ppm]: 3.699 (6.75), 3.703 (6.76), 3.708 (10.03), 3.726 (6.57), 6.836 (5.69), 6.852 (5.86), 8.188 (16.00), 8.739 (8.38), 8.755 (7.98).

### Intermediate I172

### 5-(pyrrolidin-1-yl)pyrazolo[1,5-a]pyrimidine-3-carboxylic acid

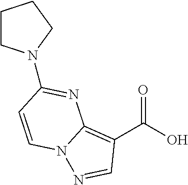 [SMILES: O=C(O)c1cnn2ccc(N3CCCC3)nc12]

Sodium hydroxide (3.7 ml, 2 M aqueous solution, 7.3 mmol) was added to a solution of ethyl 5-(pyrrolidin-1-yl)pyrazolo[1,5-a]pyrimidine-3-carboxylate (765 mg, 2.94 mmol) in tetrahydrofuran (20 ml) and methanol (5.9 ml), then stirred at 70° C. for 3 h For work-up, the mixture was concentrated, then poured into water and acidified with citric acid. The precipitate formed was collected by filtration and dried at 50° C. to give the title compound (630 mg).

¹H-NMR (500 MHz, DMSO-d6) δ[ppm]: 1.907 (0.99), 1.938 (1.66), 1.992 (1.63), 2.518 (1.58), 2.522 (1.20), 3.498 (1.69), 3.589 (1.70), 6.496 (7.19), 6.512 (7.00), 8.151 (16.00), 8.669 (8.45), 8.685 (9.19).

### Intermediate I173

### 6-(2-methoxyethyl)pyrazolo[1,5-a]pyrimidine-3-carboxylic acid

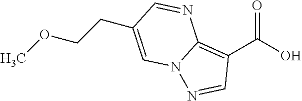 [SMILES: COCCc1cnc2c(C(=O)O)cnn2c1]

Sodium hydride (102 mg, 2.34 mmol) was added at 0° C. to a solution of 6-(2-hydroxyethyl)pyrazolo[1,5-a]pyrimidine-3-carboxylic acid (220 mg, 1.06 mmol, CAS No 725693-84-5) in dimethylformamide (4.4 ml) and the mixture was stirred for 30 minutes at room temperature. Iodomethane (200 μl, 3.2 mmol) was added and the mixture was stirred at room temperature. For work-up, the mixture was poured into ice-water, extracted with dichloromethane/isopropyl alcohol 4:1 (3×), the combined organic phases were filtrated through a silicone filter and concentrated under reduced pressure. The residue was solved in a mixture of tetrahydrofuran (4 ml). methanol (1 ml) and sodium hydroxide (1.6 ml, 2.0 M, 3.2 mmol) and the mixture was stirred at 60° C. for 3 h and then concentrated under reduced pressure. The residue was solved in water, acidified with citric acid and the mixture was extracted with dichloromethane/isopropyl alcohol 4:1 (3×), the combined organic phases were filtrated through a silicone filter and concentrated under reduced pressure to give the crude product (130 mg), which was which was used in the next step without further purification.

### Intermediate I174

### methyl 4-[8-{[tert-butyl(dimethyl)silyl]oxy}-1-oxo-2-azaspiro[4.5]dec-2-yl]-3-chlorobenzoate (isomer 1)

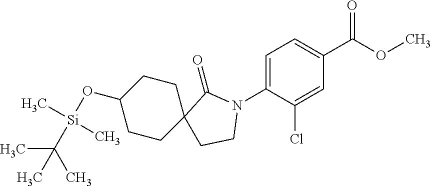 [SMILES: COC(=O)c1ccc(N2CCC3(CCC(O[Si](C)(C)C(C)(C)C)CC3)C2=O)c(Cl)c1]

To a mixture of 8-{[tert-butyl(dimethyl)silyl]oxy}-2-azaspiro[4.5]decan-1-one (331 mg, 1.17 mmol) (isomer 1) and methyl 3-chloro-4-iodobenzoate (416 mg, 1.40 mmol) in o-xylol (25 ml) was added under an argon atmosphere copper(I) iodide (44.5 mg, 234 μmol), N,N′-dimethylethylenediamine (51 μl, 470 μmol) and potassium phosphate (496 mg, 2.34 mmol) and the reaction was heated to 140° C. for 24 h in a microwave reactor. Upon cooling, the reaction mixture was filtrated through a pad of celite the residue was washed with ethyl acetate and the filtrate was concentrated under reduced pressure. Another batch was prepared accordingly, the batches were combined and the crude product was purified by flash chromatography (hexanes/ethyl acetate-gradient, 0%->60% ethyl acetate) to give the title compound as single isomer (497 mg).

LC-MS (Method 2): Rₜ=1.74 min; MS (ESIpos): m/z=452 [M+H]⁺

¹H-NMR (400 MHz, DMSO-d6) δ[ppm]: 0.831 (16.00), 0.838 (1.17), 2.027 (2.38), 2.034 (0.97), 3.623 (1.02), 3.828 (4.81), 7.519 (0.89), 7.540 (1.02), 7.894 (0.60), 7.899 (0.63), 7.919 (0.60), 7.978 (1.09), 7.983 (0.95).

### Intermediate I175

### methyl 3-chloro-4-8-hydroxy-1-oxo-2-azaspiro[4.5]dec-2-yl-benzoate (isomer 1)

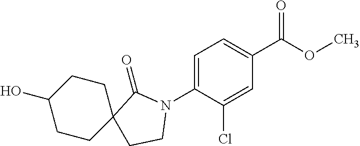 [SMILES: COC(=O)c1ccc(N2CCC3(CCC(O)CC3)C2=O)c(Cl)c1]

Tetra-N-butylammonium fluoride (2.7 ml, 1.0 M in THF, 2.7 mmol) was added to a solution of methyl4-[8-{[tert-butyl(dimethyl)silyl]oxy}-1-oxo-2-azaspiro[4.5]dec-2-yl]-3-chlorobenzoate (497 mg, 1.10 mmol) (isomer 1) in THF (9.5 ml) and the mixture was stirred at room temperature for 18 h. Tetra-N-butylammonium fluoride (2.7 ml, 1.0 M in THF, 2.7 mmol) was added and the mixture was stirred at room temperature. The mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (25 g Snap cartridge, hexanes/ethyl acetate-gradient, 50%->100% ethyl acetate) to give the title compound (178 mg) as single isomer.

LC-MS (Method 2): Rₜ=0.99 min; MS (ESIpos): m/z=338 [M+H]⁺

¹H-NMR (400 MHz, DMSO-d6) δ[ppm]: 1.291 (1.26), 1.324 (1.35), 1.555 (1.26), 1.583 (1.08), 1.667 (1.20), 1.677 (1.28), 1.700 (0.84), 1.925 (0.86), 1.935 (0.97), 1.958 (1.37), 1.987 (1.28), 2.043 (1.86), 2.060 (3.63), 2.077 (1.95), 2.322 (0.95), 2.326 (1.28), 2.331 (0.93), 2.522 (4.03), 2.664 (0.95), 2.668 (1.31), 2.673 (0.93), 3.653 (2.12), 3.670 (3.85), 3.687 (2.04), 3.736 (0.91), 3.877 (16.00), 4.398 (1.88), 4.406 (1.90), 7.570 (2.94), 7.590 (3.30), 7.943 (1.88), 7.948 (2.10), 7.964 (1.66), 7.968 (1.90), 8.026 (3.47), 8.031 (3.12).

### Intermediate I176

### methyl 3-chloro-4-[8-(1,3-dioxo-1,3-dihydro-2H-isoindol-2-yl)-1-oxo-2-azaspiro[4.5]dec-2-yl]benzoate (isomer 1)

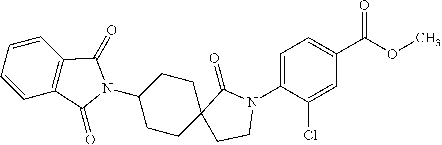 [SMILES: COC(=O)c1ccc(N2CCC3(CCC(N4C(=O)c5ccccc5C4=O)CC3)C2=O)c(Cl)c1]

Diisopropyl azodicarboxylate (130 μl, 680 μmol) was added drop wise to a mixture of methyl 3-chloro-4-[8-hydroxy-1-oxo-2-azaspiro[4.5]dec-2-yl]benzoate (306 mg, 50% purity, 453 μmol) (isomer 1), 1H-isoindole-1,3(2H)-dione (100 mg, 679 μmol) and triphenylphosphine (178 mg, 679 μmol) in THF (7.4 ml) and the mixture was stirred at room temperature for 48 hours. For work-up, the reaction mixture was concentrated and the residue was purified by flash chromatography (hexanes/ethyl acetate gradient, 20%->50% ethyl acetate to give the title compound (81 mg) as single isomer.

LC-MS (Method 2): Rₜ=1.31 min; MS (ESIpos): m/z=467 [M+H]⁺

¹H-NMR (400 MHz, DMSO-d6) δ[ppm]: 1.667 (1.00), 1.677 (1.13), 1.708 (1.04), 1.756 (2.15), 2.207 (1.53), 2.224 (3.21), 2.242 (1.74), 2.323 (0.88), 2.327 (1.25), 2.332 (0.81), 2.518 (4.07), 2.523 (2.87), 2.665 (0.76), 2.669 (1.06), 2.674 (0.72), 3.728 (1.71), 3.746 (3.00), 3.762 (1.64), 3.883 (16.00), 7.594 (3.03), 7.615 (3.22), 7.825 (0.97), 7.837 (1.80), 7.842 (1.89), 7.848 (7.21), 7.855 (8.07), 7.861 (2.59), 7.867 (1.83), 7.872 (0.67), 7.878 (0.92), 7.961 (2.01), 7.965 (2.24), 7.981 (1.73), 7.986 (2.03), 8.043 (3.65), 8.048 (3.30).

### Intermediate I177

### methyl 4-[8-amino-1-oxo-2-azaspiro[4.5]dec-2-yl]-3-chlorobenzoate (isomer 1)

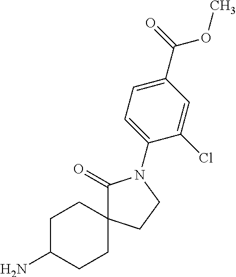 [SMILES: COC(=O)c1ccc(N2CCC3(CCC(N)CC3)C2=O)c(Cl)c1]

A mixture of methyl 3-chloro-4-[8-(1,3-dioxo-1,3-dihydro-2H-isoindol-2-yl)-1-oxo-2-azaspiro[4.5]dec-2-yl]benzoate (79.0 mg, 169 μmol) (isomer 1) and hydrazine hydrate (42 μl, 850 μmol) in ethanol (1.4 ml) was stirred at 80° C. f or 3 h. Upon cooling, the precipitate was filtrated off, washed with ethanol and the filtrate was concentrated to give the title compound (69 mg), which was used in the next step without further purification.

LC-MS (Method 2): Rₜ=0.99 min; MS (ESIpos): m/z=337 [M+H]⁺

### Intermediate I178

### 8-{[tert-butyl(dimethyl)silyl]oxy}-2-(thiophen-2-yl)-2-azaspiro[4.5]decan-1-one (isomer 1)

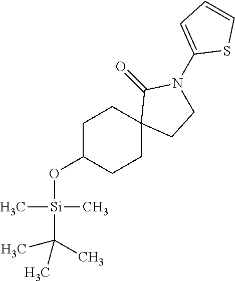 [SMILES: CC(C)(C)[Si](C)(C)OC1CCC2(CC1)CCN(c1cccs1)C2=O]

To a mixture of 8-{[tert-butyl(dimethyl)silyl]oxy}-2-azaspiro[4.5]decan-1-one (500 mg, 1.76 mmol) (isomer 1) and 2-iodothiophene (230 μl, 2.1 mmol) in toluene (12 ml) was added under an argon atmosphere copper(I) iodide (67.2 mg, 353 μmol), N,N′-imethylethylenediamine (77 μl, 710 μmol) and potassium phosphate (749 mg, 3.53 mmol) and the reaction was heated to 80° C. for 18 h in a microwave reactor. Upon cooling, the reaction mixture was filtrated through a pad of celite and the filtrate was concentrated under reduced pressure. The crude product was purified by flash chromatography (hexanes/ethyl acetate-gradient, 0%->20% ethyl acetate) to give the title compound as single isomer (604 mg).

LC-MS (Method 2): Rₜ=1.67 min; MS (ESIneg): m/z=366 [M+H]⁻

¹H-NMR (400 MHz, DMSO-d6) δ[ppm]: 0.852 (16.00), 3.748 (0.92), 6.595 (0.64), 6.845 (0.63), 6.855 (0.58), 6.859 (0.68), 6.869 (0.62), 7.000 (0.65), 7.004 (0.72), 7.014 (0.62).

### Intermediate I179

### 8-hydroxy-2-(thiophen-2-yl)-2-azaspiro[4.5]decan-1-one (isomer 1)

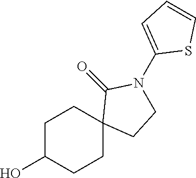 [SMILES: O=C1N(c2cccs2)CCC12CCC(O)CC2]

Tetra-N-butylammonium fluoride (4.1 ml, 1.0 M in THF, 4.1 mmol) was added to a solution of 8-{[tert-butyl(dimethyl)silyl]oxy}-2-(thiophen-2-yl)-2-azaspiro[4.5]decan-1-one (602 mg, 1.65 mmol) (isomer 1) in THF (14 ml) and the mixture was stirred at room temperature for 18 h. The mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (25 g Snap cartridge, hexanes/ethyl acetate-gradient, 50%->100% ethyl acetate) to give the title compound (178 mg) as single isomer.

LC-MS (Method 2): Rₜ=0.92 min; MS (ESIneg): m/z=252 [M+H]⁻

¹H-NMR (400 MHz, DMSO-d6) δ[ppm]: 0.883 (0.65), 1.216 (0.82), 1.268 (2.80), 1.279 (5.52), 1.290 (3.36), 1.301 (3.32), 1.311 (5.87), 1.323 (3.23), 1.543 (1.55), 1.552 (2.50), 1.561 (1.73), 1.572 (2.46), 1.579 (4.96), 1.586 (5.18), 1.594 (2.76), 1.605 (3.15), 1.613 (4.44), 1.622 (2.59), 1.687 (2.07), 1.698 (5.13), 1.711 (5.43), 1.722 (3.36), 1.732 (3.54), 1.745 (3.19), 1.757 (1.25), 1.941 (4.05), 1.952 (4.27), 1.970 (4.74), 1.974 (5.56), 1.979 (5.13), 1.984 (4.74), 2.002 (3.45), 2.012 (3.02), 2.032 (1.47), 2.079 (8.93), 2.089 (2.29), 2.096 (14.62), 2.105 (2.29), 2.114 (9.49), 2.367 (0.82), 2.371 (1.16), 2.376 (0.82), 2.563 (4.10), 2.567 (2.63), 2.709 (0.86), 2.713 (1.16), 2.718 (0.82), 3.781 (3.71), 3.788 (3.71), 3.794 (2.93), 3.812 (11.00), 3.821 (2.33), 3.830 (16.00), 3.837 (2.33), 3.847 (10.05), 4.455 (8.97), 4.463 (9.14), 6.667 (8.84), 6.671 (9.40), 6.677 (9.88), 6.680 (9.62), 6.932 (9.32), 6.942 (8.67), 6.946 (10.35), 6.955 (9.79), 7.080 (10.39), 7.083 (10.78), 7.094 (8.67), 7.097 (8.67).

### Intermediate I180

### 2-[1-oxo-2-(thiophen-2-yl)-2-azaspiro[4.5]dec-8-yl]-1H-isoindole-1,3(2H)-dione (isomer 1)

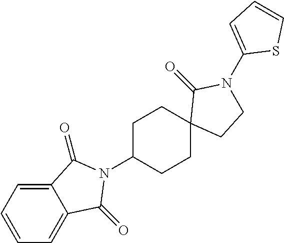 [SMILES: O=C1c2ccccc2C(=O)N1C1CCC2(CC1)CCN(c1cccs1)C2=O]

Diisopropyl azodicarboxylate (410 μl, 2.1 mmol) was added drop wise at 0° C. to a mixture of 8-hydroxy-2-(thiophen-2-yl)-2-azaspiro[4.5]decan-1-one (353 mg, 1.40 mmol) (isomer 1), 1H-isoindole-1,3(2H)-dione (310 mg, 2.11 mmol) and triphenylphosphine (553 mg, 2.11 mmol) in THF (17 ml) and the mixture was stirred at room temperature for 18 hours. Triphenylphosphine (553 mg, 2.11 mmol) and diisopropyl azodicarboxylate (410 μl, 2.1 mmol) were added and the mixture was stirred for 4 hours at room temperature. For work-up, the reaction mixture was concentrated and the residue was purified by flash chromatography (hexanes/ethyl acetate gradient, 20%->40% ethyl acetate to give after trituration with methanol the title compound (167 mg) as single isomer.

LC-MS (Method 2): Rₜ=1.25 min; MS (ESIneg): m/z=381 [M+H]⁻

¹H-NMR (400 MHz, DMSO-d6) δ[ppm]: 1.657 (2.22), 1.667 (3.00), 1.686 (4.87), 1.695 (7.84), 1.711 (2.57), 1.742 (2.36), 1.751 (2.02), 1.987 (0.61), 2.192 (4.12), 2.209 (6.31), 2.227 (5.05), 2.238 (2.13), 2.254 (1.67), 2.268 (1.70), 2.327 (0.72), 2.518 (2.68), 2.523 (1.70), 2.669 (0.75), 3.849 (3.83), 3.859 (1.15), 3.868 (5.94), 3.875 (1.15), 3.884 (3.75), 4.017 (0.98), 4.038 (0.95), 4.047 (1.70), 4.057 (0.92), 4.078 (0.81), 6.672 (3.83), 6.675 (3.92), 6.681 (4.41), 6.685 (3.98), 6.908 (4.38), 6.918 (3.89), 6.922 (4.50), 6.931 (4.55), 7.064 (4.99), 7.068 (4.67), 7.078 (4.18), 7.081 (4.18), 7.823 (2.45), 7.830 (1.67), 7.834 (3.86), 7.841 (4.24), 7.846 (15.14), 7.854 (16.00), 7.860 (5.28), 7.866 (4.01), 7.870 (1.73), 7.877 (2.34).

### Intermediate I181

### 8-amino-2-(thiophen-2-yl)-2-azaspiro[4.5]decan-1-one (isomer 1)

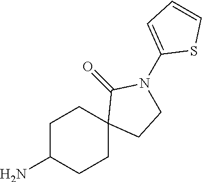 [SMILES: NC1CCC2(CC1)CCN(c1cccs1)C2=O]

A mixture of 2-[(1-oxo-2-(thiophen-2-yl)-2-azaspiro[4.5]dec-8-yl]-1H-isoindole-1,3(2H)-dione (158 mg, 415 μmol) (isomer 1) and hydrazine hydrate (130 μl, 80% purity, 2.1 mmol) in ethanol (2.7 ml) was stirred at 80° C. for 3 h. Upon cooling, the precipitate was filtrated off, washed with ethanol and the filtrate was concentrated. The residue was suspended in dichloromethane, the precipitate was filtrated off. Water was added to the filtrate and the organic phase was filtrated over a hydrophobic filter and concentrated to give the title compound (68 mg).

LC-MS (Method 2): Rₜ=0.88 min; MS (ESIneg): m/z=251 [M+H]⁻

¹H-NMR (400 MHz, DMSO-d6) δ[ppm]: 1.072 (1.60), 1.083 (1.65), 1.104 (4.40), 1.115 (4.19), 1.132 (4.58), 1.143 (4.77), 1.165 (2.49), 1.175 (2.37), 1.231 (1.95), 1.482 (2.51), 1.494 (2.19), 1.508 (7.40), 1.516 (15.74), 1.526 (11.51), 1.549 (6.51), 1.557 (7.26), 1.581 (1.95), 1.591 (2.09), 1.696 (5.23), 1.705 (5.33), 1.717 (3.00), 1.729 (5.05), 1.739 (4.58), 1.781 (1.16), 1.928 (0.79), 1.940 (0.79), 2.053 (10.40), 2.062 (2.93), 2.071 (15.51), 2.079 (2.79), 2.084 (3.35), 2.088 (10.77), 2.323 (1.12), 2.327 (1.49), 2.331 (1.09), 2.518 (5.81), 2.523 (4.02), 2.527 (3.00), 2.537 (4.58), 2.547 (2.49), 2.555 (1.53), 2.565 (2.16), 2.575 (1.09), 2.665 (1.09), 2.669 (1.47), 2.674 (1.05), 3.779 (11.70), 3.788 (2.67), 3.797 (16.00), 3.805 (2.77), 3.815 (11.23), 6.635 (9.74), 6.638 (10.28), 6.645 (10.86), 6.648 (10.44), 6.891 (10.26), 6.900 (9.49), 6.904 (12.09), 6.913 (11.56), 7.039 (10.93), 7.042 (11.79), 7.052 (9.70), 7.056 (9.16), 8.519 (0.77).

### Intermediate I182

### ammonium 6-(2-hydroxypropan-2-yl)-7-methylpyrazolo[1,5-a]pyrimidine-3-carboxylate

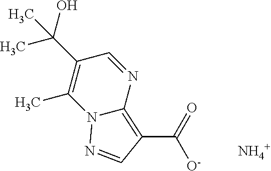 [SMILES: Cc1c(C(C)(C)O)cnc2c(C(=O)[O-])cnn12 ; [NH4+]]

To a suspension of 6-acetyl-7-methylpyrazolo[1,5-a]pyrimidine-3-carboxylic acid (150 mg, 684 μmol, CAS 774183-58-3) in THF (7.5 ml) was added bromo(methyl)magnesium (2.7 ml, 1.0 M, 2.7 mmol) at 0° C. and the mixture was stirred for 49 hours at room temperature. For work-up a solution of ammonium chloride was added and the mixture was extracted with dichloromethane/2-propanol (4:1). The aqueous phase was acidified with hydrochloric acid and was extracted with dichloromethane/2-propanol (4:1). The combined organic phases were filtrated over a hydrophobic filter and concentrated to give the title compound (60 mg).

### Intermediate I183

### 6-acetyl-N-[(2-(2-chloro-4-fluorophenyl)-1-oxo-2-azaspiro[4.5]dec-8-yl]-7-methylpyrazolo[1,5-a]pyrimidine-3-carboxamide (isomer 1)

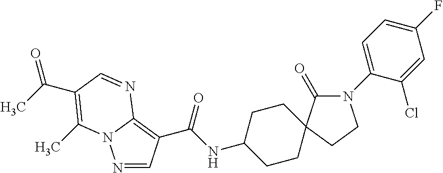 [SMILES: CC(=O)c1cnc2c(C(=O)NC3CCC4(CC3)CCN(c3ccc(F)cc3Cl)C4=O)cnn2c1C]

To a mixture of 6-acetyl-7-methylpyrazolo[1,5-a]pyrimidine-3-carboxylic acid (336 mg, 1.53 mmol, CAS 774183-58-3) and 8-amino-2-(2-chloro-4-fluorophenyl)-2-azaspiro[4.5]decan-1-one (500 mg, 1.68 mmol) in DMF (6.4 ml) was added PyBOP (877 mg, 1.68 mmol) and N,N-diisopropylethylamine (1.3 ml, 7.7 mmol) and the reaction was stirred for 3 days at room temperature. For work-up the reaction was poured into water and the resulting precipitate was filtrated. The residue washed with i-propanol to give after drying the title product (633 mg) which was used in the subsequent steps without further purification.

LC-MS (Method 2): Rₜ=1.13 min; MS (ESIneg): m/z=497 [M−H]⁺

¹H-NMR (400 MHz, DMSO-d6) δ[ppm]: 1.682 (2.11), 1.691 (2.33), 1.707 (1.53), 1.713 (1.28), 2.150 (1.29), 2.167 (2.42), 2.184 (1.35), 2.730 (16.00), 3.061 (10.67), 3.616 (1.39), 3.634 (2.39), 3.650 (1.30), 7.290 (0.60), 7.297 (0.62), 7.312 (0.93), 7.319 (1.03), 7.332 (0.70), 7.340 (0.73), 7.467 (1.28), 7.482 (1.35), 7.490 (1.07), 7.504 (0.99), 7.580 (1.31), 7.587 (1.30), 7.601 (1.29), 7.608 (1.29), 7.893 (1.13), 7.913 (1.07), 8.734 (6.89), 9.244 (4.69).

### Intermediate I184

### ethyl-4-{[tert-butyl(dimethyl)silyl]oxy}-1-(2-methylprop-2-en-1-yl)cyclohexanecarboxylate (mixture of cis/trans isomers)

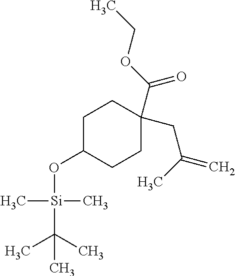 [SMILES: C=C(C)CC1(C(=O)OCC)CCC(O[Si](C)(C)C(C)(C)C)CC1]

Lithium diisopropylamide in THF (21 ml, 2.0 M, 42 mmol) was added drop wise to a solution of ethyl-4-{[tert-butyl(dimethyl)silyl]oxy}cyclohexanecarboxylate (10.0 g, 34.9 mmol) in THF (47 ml) at −78° C. and the mixture was stirred for 30 min at that temperature. 3-bromo-2-methylprop-1-ene (5.4 ml, 97% purity, 52 mmol) was added and the mixture was stirred for 1 h at −78° C. and then warmed during 2 h to room temperature and the mixture was stirred for 18 hours at room temperature. For work-up, ice water was added and the mixture was extracted with tert-butyl methyl ether (3×). The combined organic phases were washed with brine and filtrated through a silicone filter and concentrated under reduced pressure. The crude product (12 g) was purified by flash chromatography (340 g Snap cartridge, hexanes/ethyl acetate gradient) to give the title product as a mixture of isomers (9.5 g).

### Intermediate I185

### ethyl 4-{[tert-butyl(dimethyl)silyl]oxy}-1-(2-oxopropyl)cyclohexanecarboxylate (mixture of cis/trans isomers)

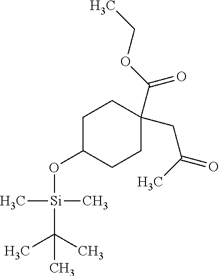 [SMILES: CCOC(=O)C1(CC(C)=O)CCC(O[Si](C)(C)C(C)(C)C)CC1]

A solution of ethyl 4-{[tert-butyl(dimethyl)silyl]oxy}-1-(2-methylprop-2-en-1-yl)cyclohexanecarboxylate (mixture of cis/trans isomers) (6.45 g, 18.9 mmol) in dichloromethane (270 ml) was purged with ozone at −78° C. for 35 minutes. The reaction was stirred for additional 15 minutes at −78° C. Dimeth yl sulfide (14 ml, 190 mmol) was added dropwise and the mixture was warmed to room temperature. For work-up the mixture was poured into water and was extracted with dichloromethane (3×). The combined organic phases were washed with brine and filtrated through a silicone filter and concentrated under reduced pressure. The crude product was purified by flash chromatography (hexanes/ethyl acetate gradient) to give the title product (1.42 g).

¹H-NMR (400 MHz, DMSO-d6) δ[ppm]: 0.821 (1.54), 0.828 (16.00), 0.835 (1.34), 1.091 (1.53), 1.109 (3.17), 1.126 (1.57), 2.017 (5.69), 2.712 (2.46), 3.995 (1.49), 4.012 (1.46).

### Intermediate I186

### ethyl 4-{[tert-butyl(dimethyl)silyl]oxy}-1-[2-(hydroxyimino)propyl]cyclohexanecarboxylate (mixture of cis/trans isomers)

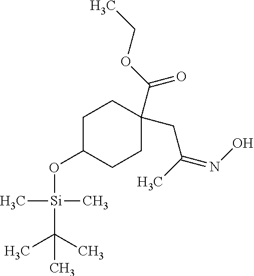 [SMILES: CCOC(=O)C1(C/C(C)=N\O)CCC(O[Si](C)(C)C(C)(C)C)CC1]

To a solution of ethyl 4-{[tert-butyl(dimethyl)silyl]oxy}-1-(2-oxopropyl)cyclohexanecarboxylate (mixture of cis/trans isomers) (2.12 g, 6.18 mmol) in methanol (11 ml) was added triethylamine (2.6 ml, 19 mmol) and the mixture was stirred for 5 minutes at room temperature. hydroxylamine hydrochloride (859 mg, 12.4 mmol) was added and the reaction was stirred for 16 hours at 70° C. For work-up the mixture was concentrated under reduced pressure and residue was purified by flash chromatography (hexanes/ethyl acetate gradient 20%->50% ethyl acetate) to give the title product as a mixture of isomers (982 mg).

¹H-NMR (500 MHz, DMSO-d6) δ[ppm]: 0.000 (13.30), 0.004 (1.44), 0.816 (4.99), 0.819 (16.00), 0.843 (1.14), 1.151 (1.67), 1.165 (3.50), 1.179 (2.60), 1.629 (5.74), 1.661 (1.48), 2.059 (4.94), 2.276 (1.89), 2.503 (0.89), 4.045 (1.49), 4.059 (1.53).

### Intermediate I187

### 8-{[tert-butyl(dimethyl)silyl]oxy}-3-methyl-2-azaspiro[4.5]decan-1-one (mixture of isomers)

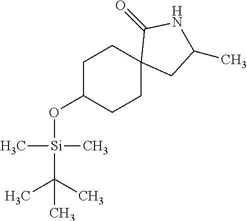

An autoclave was charged with a mixture of ethyl 4-{[tert-butyl(dimethyl)silyl]oxy}-1-[2-(hydroxyimino)propyl]cyclohexanecarboxylate (mixture of cis/trans isomers) (980 mg, 2.74 mmol) and Pd/C (10%) (171 mg) in methanol (20 ml) and then pressurized with hydrogen (6.2 bar). The mixture was stirred at 80° C. for 45 h ours. Upon cooling, the catalyst was filtrated off, washed with methanol and the filtrate was concentrated under reduced pressure.

The residue was dissolved in toluene (7.2 ml), triethylamine (760 μl, 5.5 mmol) was added and the mixture was refluxed for 24 h. For work-up, the reaction mixture was concentrated and the residue was purified by flash chromatography (hexanes/ethyl acetate gradient) to give the title compound (329 mg) as mixture of isomers.

LC-MS (Method 2): Rₜ=1.48 min; MS (ESIpos): m/z=298 [M+H]⁺

1H-NMR (400 MHz, DMSO-d6): δ [ppm]=7.66-7.31 (m, 1H), 3.86 (br s, 1H), 3.59-3.42 (m, 1H), 2.15 (dd, 1H), 1.95 (td, 1H), 1.77-1.67 (m, 1H), 1.66-1.56 (m, 1H), 1.52 (dt, 2H), 1.45-1.34 (m, 1H), 1.27-1.19 (m, 1H), 1.18-1.10 (m, 1H), 1.08-0.98 (m, 4H), 0.90-0.79 (m, 10H), 0.04-0.08 (m, 6H).

### Intermediate I188

### 8-{[tert-butyl(dimethyl)silyl]oxy}-2-(2-chloro-4-fluorophenyl)-3-methyl-2-azaspiro[4.5]decan-1-one (mixture of isomers)

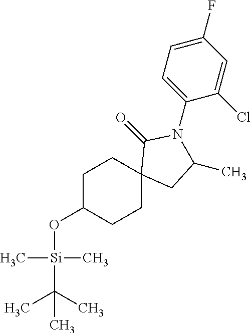 [SMILES: CC1CC2(CCC(O[Si](C)(C)C(C)(C)C)CC2)C(=O)N1c1ccc(F)cc1Cl]

To a mixture of 8-{[tert-butyl(dimethyl)silyl]oxy}-3-methyl-2-azaspiro[4.5]decan-1-one (mixture of isomers) (329 mg, 1.11 mmol) and 2-chloro-4-fluoro-1-iodobenzene (340 mg, 1.33 mmol) in o-xylol (20 ml) was added under an argon atmosphere copper(I) iodide (42.1 mg, 221 μmol), N,N′-imethylethylenediamine (48 μl, 440 μmol) and potassium phosphate (469 mg, 2.21 mmol) and the reaction was heated to 140° C. for 24 h in a microwave reactor. Upon cooling, the reaction mixture was filtrated through a pad of celite the residue was washed with ethyl acetate and the filtrate was concentrated under reduced pressure. The crude product was purified by flash chromatography (hexanes/ethyl acetate-gradient, 0%->10% ethyl acetate) to give the title compound (280 mg).

LC-MS (Method 2): Rₜ=1.75 min; MS (ESIpos): m/z=426 [M+H]⁺

1H-NMR (400 MHz, DMSO-d6): δ [ppm]=7.55 (dd, 1H), 7.44-7.33 (m, 1H), 7.31-7.16 (m, 1H), 4.06-3.97 (m, 1H), 3.94-3.85 (m, 1H), 2.12-1.98 (m, 1H), 1.92-1.76 (m, 1H), 1.71-1.36 (m, 6H), 1.26-1.14 (m, 1H), 0.98 (d, 3H), 0.88-0.80 (m, 9H), 0.01 (d, 6H)

### Intermediate I189

### 2-(2-chloro-4-fluorophenyl)-8-hydroxy-3-methyl-2-azaspiro[4.5]decan-1-one (mixture of isomers)

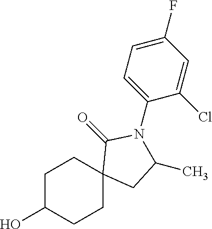 [SMILES: CC1CC2(CCC(O)CC2)C(=O)N1c1ccc(F)cc1Cl]

Tetra-N-butylammonium fluoride (1.9 ml, 1.0 M, 1.9 mmol) was added to a solution of 8-{[tert-butyl(dimethyl)silyl]oxy}-2-(2-chloro-4-fluorophenyl)-3-methyl-2-azaspiro[4.5]decan-1-one (mixture of isomers) (275 mg, 645 μmol) in THF (5.6 ml) and the mixture was stirred at room temperature for 18 h. The mixture was poured into water, extracted with ethyl acetate (3×) and the combined organic phases were washed with sodium bicarbonate solution and brine, filtrated through a silicone filter and concentrated under reduced pressure. The residue was purified by flash chromatography (hexanes/ethyl acetate-gradient, 20%->100% ethyl acetate) to give the title compound (122 mg).

LC-MS (Method 2): Rₜ=1.08 min; MS (ESIpos): m/z=312 [M+H]⁺

¹H-NMR (400 MHz, DMSO-d6) δ[ppm]: 0.999 (15.70), 1.015 (16.00), 1.154 (0.70), 1.173 (1.79), 1.177 (0.96), 1.191 (2.02), 1.201 (1.13), 1.210 (1.10), 1.221 (1.86), 1.234 (1.29), 1.346 (0.86), 1.355 (1.73), 1.366 (1.06), 1.379 (1.06), 1.389 (1.89), 1.399 (1.06), 1.458 (2.99), 1.479 (4.12), 1.489 (3.78), 1.505 (1.56), 1.511 (3.75), 1.521 (0.93), 1.579 (1.39), 1.588 (2.76), 1.602 (4.32), 1.614 (4.75), 1.622 (2.09), 1.709 (1.49), 1.720 (1.63), 1.732 (1.13), 1.743 (1.29), 1.754 (1.16), 1.831 (1.06), 1.844 (1.13), 1.864 (1.43), 1.876 (1.03), 1.887 (0.93), 1.901 (0.73), 1.988 (2.46), 2.028 (1.26), 2.037 (1.46), 2.059 (1.89), 2.066 (1.73), 2.088 (1.23), 2.099 (0.96), 2.331 (1.53), 2.337 (0.73), 2.354 (2.52), 2.370 (2.76), 2.385 (2.56), 2.402 (2.42), 2.518 (8.20), 2.523 (5.58), 2.674 (1.46), 2.678 (0.66), 3.739 (2.32), 4.000 (0.73), 4.018 (1.63), 4.035 (1.73), 4.053 (0.90), 4.378 (7.27), 4.386 (7.20), 7.273 (1.96), 7.280 (2.16), 7.295 (3.82), 7.302 (4.22), 7.315 (2.95), 7.323 (3.09), 7.392 (1.39), 7.567 (3.95), 7.574 (3.92), 7.588 (3.98), 7.596 (3.78).

### Intermediate I190

### 2-[2-(2-chloro-4-fluorophenyl)-3-methyl-1-oxo-2-azaspiro[4.5]dec-8-yl]-1H-isoindole-1,3(2H)-dione (trans isomer, mixture of isomers at C₃)

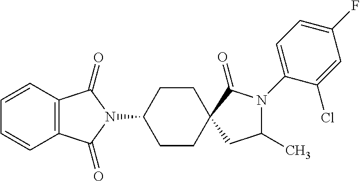

Diisopropyl azodicarboxylate (110 μl, 580 μmol) was added drop wise to a mixture of 2-(2-chloro-4-fluorophenyl)-8-hydroxy-3-methyl-2-azaspiro[4.5]decan-1-one (mixture of isomers) (120 mg, 385 μmol), 1H-isoindole-1,3(2H)-dione (84.9 mg, 577 μmol) and triphenylphosphine (100 mg, 384 μmol) in THF (6.3 ml) and the mixture was stirred at room temperature for 20 h. Triphenylphosphine(151 mg, 577 μmol) was added and the mixture was stirred for 24 h at room temperature. For work-up, the reaction mixture was concentrated and the residue was purified by flash chromatography (hexanes/ethyl acetate gradient, 0%->50% ethyl acetate to give the title compound (85 mg).

LC-MS (Method 2): Rₜ=1.36 min; MS (ESIpos): m/z=441 [M+H]⁺

### Intermediate I191

### 8-amino-2-(2-chloro-4-fluorophenyl)-3-methyl-2-azaspiro[4.5]decan-1-one (trans isomer, mixture of isomers at C3)

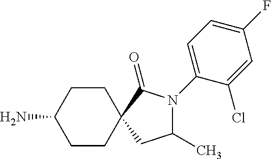

A mixture of 2-[2-(2-chloro-4-fluorophenyl)-3-methyl-1-oxo-2-azaspiro[4.5]dec-8-yl]-1H-isoindole-1,3(2H)-dione (trans isomer, mixture of isomers at C3) (85.0 mg, 193 μmol) and hydrazine hydrate (48 μl, 98% purity, 960 μmol) in ethanol (1.6 ml) was stirred at 80° C. for 3 h. Upon cooling, the precipitate was filtrated off, washed with ethanol and the filtrate was concentrated and the residue was dissolved in dichloromethane. The mixture was washed with water and the organic phase was filtrated over a hydrophobic filter and concentrated to give the title compound (33 mg).

¹H-NMR (400 MHz, DMSO-d6) δ[ppm]: 1.014 (14.95), 1.029 (16.00), 1.067 (1.41), 1.077 (0.87), 1.133 (1.09), 1.170 (8.34), 1.186 (8.80), 1.218 (1.09), 1.230 (1.64), 1.246 (0.64), 1.364 (0.96), 1.373 (1.09), 1.397 (1.73), 1.406 (1.91), 1.421 (1.69), 1.428 (2.51), 1.435 (3.24), 1.456 (3.42), 1.459 (3.28), 1.467 (2.92), 1.488 (1.96), 1.641 (2.05), 1.648 (2.10), 1.657 (1.19), 1.674 (2.10), 1.686 (3.78), 1.695 (4.65), 1.722 (6.11), 1.730 (3.65), 1.739 (2.01), 1.754 (1.91), 1.764 (1.28), 2.318 (0.77), 2.337 (0.82), 2.414 (2.14), 2.431 (2.51), 2.446 (2.42), 2.463 (2.96), 2.518 (10.26), 2.523 (7.89), 2.660 (0.82), 2.679 (0.82), 4.019 (1.09), 4.766 (0.68), 7.273 (1.82), 7.280 (2.14), 7.294 (3.51), 7.302 (3.78), 7.315 (2.87), 7.322 (3.19), 7.388 (1.37), 7.566 (3.65), 7.573 (3.78), 7.588 (3.74), 7.595 (3.74).

### Intermediate I192

### 8-{[tert-butyl(dimethyl)silyl]oxy}-2-[2-chloro-4-(trifluoromethoxy)phenyl]-2-azaspiro[4.5]decan-1-one (isomer 1)

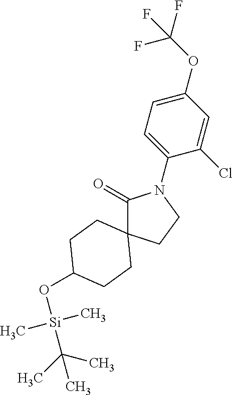 [SMILES: CC(C)(C)[Si](C)(C)OC1CCC2(CC1)CCN(c1ccc(OC(F)(F)F)cc1Cl)C2=O]

To a mixture of 8-{[tert-butyl(dimethyl)silyl]oxy}-2-azaspiro[4.5]decan-1-one (500 mg, 1.76 mmol) (isomer 1) and 3-chloro-4-iodophenyl trifluoromethyl ether (696 mg, 98% purity, 2.12 mmol) in o-xylol (38 ml) was added under an argon atmosphere copper(I) iodide (67.2 mg, 353 μmol), N,N′-dimethylethylenediamine (77 μl, 710 μmol) and potassium phosphate (749 mg, 3.53 mmol) and the reaction was heated to 140° C. for 48 h in a microwave reactor. Upon cooling, the reaction mixture was filtrated through a pad of celite the residue was washed with ethyl acetate and the filtrate was concentrated under reduced pressure. Another batch was prepared accordingly, the batches were combined and the crude product was purified by flash chromatography (hexanes/ethyl acetate-gradient, 50%->100% ethyl acetate) to give the title compound as single isomer (1.0 g).

LC-MS (Method 2): Rₜ=1.78 min; MS (ESIpos): m/z=478 [M+H]⁺

1H-NMR (400 MHz, DMSO-d6): δ [ppm]=7.71 (d, 1H), 7.62-7.36 (m, 2H), 3.94 (br s, 1H), 3.62 (t, 2H), 2.07 (t, 2H), 2.01-1.83 (m, 2H), 1.71-1.54 (m, 4H), 1.33 (br d, 2H), 0.88 (s, 9H), 0.05 (s, 6H).

### Intermediate I193

### 2-[2-chloro-4-(trifluoromethoxy)phenyl]-8-hydroxy-2-azaspiro[4.5]decan-1-one (isomer 1)

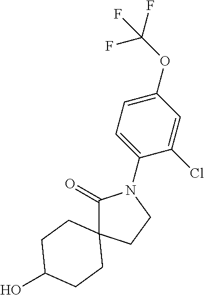 [SMILES: O=C1N(c2ccc(OC(F)(F)F)cc2Cl)CCC12CCC(O)CC2]

Tetra-N-butylammonium fluoride (5.3 ml, 1.0 M in THF, 5.3 mmol) was added to a solution of 8-{[tert-butyl(dimethyl)silyl]oxy}-2-[2-chloro-4-(trifluoromethoxy)phenyl]-2-azaspiro[4.5]decan-1-one (1.02 g, 2.13 mmol) (isomer 1) in THF (18 ml) and the mixture was stirred at room temperature for 18 h. Tetra-N-butylammonium fluoride (2.12 ml, 1.0 M in THF, 2.12 mmol) was added. Upon complete conversion, the mixture was poured into water, extracted with ethyl acetate (3×) and the combined organic phases were washed with saturated sodium bicarbonate solution and brine, filtrated through a silicone filter and concentrated under reduced pressure. The residue was purified by flash chromatography (hexanes/ethyl acetate-gradient, 50%->100% ethyl acetate) to give the title compound (593 mg) as single isomer.

LC-MS (Method 2): Rₜ=1.16 min; MS (ESIpos): m/z=364 [M+H]⁺

¹H-NMR (400 MHz, DMSO-d6) δ[ppm]: 1.154 (0.95), 1.172 (1.90), 1.190 (0.95), 1.271 (2.99), 1.282 (5.53), 1.295 (3.76), 1.303 (3.67), 1.316 (5.85), 1.327 (3.35), 1.517 (2.86), 1.526 (2.04), 1.545 (5.30), 1.551 (5.48), 1.570 (3.49), 1.578 (4.76), 1.587 (2.86), 1.650 (2.27), 1.662 (5.26), 1.673 (5.62), 1.684 (3.63), 1.695 (3.67), 1.708 (3.17), 1.719 (1.36), 1.919 (3.81), 1.929 (4.17), 1.952 (5.94), 1.980 (3.54), 1.988 (6.16), 2.031 (7.89), 2.048 (15.32), 2.065 (8.25), 2.074 (1.45), 2.322 (1.90), 2.327 (2.63), 2.331 (1.95), 2.523 (9.34), 2.665 (2.04), 2.669 (2.67), 2.673 (1.95), 3.603 (8.88), 3.611 (2.76), 3.620 (16.00), 3.629 (2.86), 3.637 (8.39), 3.741 (3.76), 4.017 (0.86), 4.035 (0.82), 4.396 (7.66), 4.404 (7.80), 5.759 (7.98), 7.449 (2.99), 7.452 (3.44), 7.456 (3.49), 7.471 (4.71), 7.474 (5.21), 7.478 (5.44), 7.480 (4.71), 7.550 (15.68), 7.571 (9.84), 7.710 (8.97), 7.716 (8.34).

### Intermediate I194

### 2-{2-[2-chloro-4-(trifluoromethoxy)phenyl]-1-oxo-2-azaspiro[4.5]dec-8-yl}-1H-isoindole-1,3(2H)-dione (isomer 1)

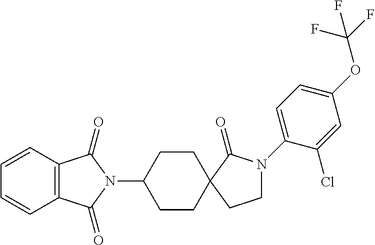 [SMILES: O=C1c2ccccc2C(=O)N1C1CCC2(CC1)CCN(c1ccc(OC(F)(F)F)cc1Cl)C2=O]

Diisopropyl azodicarboxylate (480 μl, 2.4 mmol) was added drop wise to a mixture of 2-[2-chloro-4-(trifluoromethoxy)phenyl]-8-hydroxy-2-azaspiro[4.5]decan-1-one (593 mg, 1.63 mmol) (isomer 1), 1H-isoindole-1,3(2H)-dione (360 mg, 2.45 mmol) and triphenylphosphine (641 mg, 2.45 mmol) in THF (26 ml) and the mixture was stirred at room temperature for 48 hours. For work-up, the reaction mixture was concentrated and the residue was purified by flash chromatography (hexanes/ethyl acetate gradient, 20%->50% ethyl acetate) flowed by HPLC purification (Method 9) to give the title compound (142 mg).

LC-MS (Method 2): Rₜ=1.43 min; MS (ESIpos): m/z=493 [M+H]⁺

### Intermediate I195

### 8-amino-2-[2-chloro-4-(trifluoromethoxy)phenyl]-2-azaspiro[4.5]decan-1-one (isomer 1)

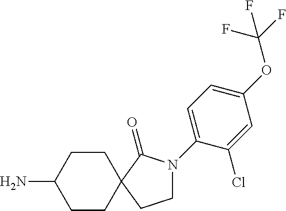 [SMILES: NC1CCC2(CC1)CCN(c1ccc(OC(F)(F)F)cc1Cl)C2=O]

A mixture of 2-{2-[2-chloro-4-(trifluoromethoxy)phenyl]-1-oxo-2-azaspiro[4.5]dec-8-yl}-1H-isoindole-1,3(2H)-dione (162 mg, 329 μmol) (isomer 1) and hydrazine hydrate (82 μl, 1.6 mmol) in ethanol (2.8 ml, 48 mmol) was stirred at 80° C. for 3 h. Upon cooling, the precipitate was filtrated off, washed with ethanol and the filtrate was concentrated to give the title compound (131 mg), which was used in the next step without further purification.

LC-MS (Method 2): Rₜ=1.18 min; MS (ESIpos): m/z=363 [M+H]⁺

¹H-NMR (400 MHz, DMSO-d6) δ[ppm]: 1.006 (4.99), 1.024 (9.01), 1.041 (4.90), 1.141 (2.86), 1.157 (2.77), 2.040 (1.44), 2.045 (16.00), 2.490 (1.60), 2.494 (1.30), 3.400 (1.22), 3.417 (1.16), 3.599 (1.14), 5.729 (3.93), 7.514 (1.39), 7.536 (0.86).

### Intermediate I196

### 8-{[tert-butyl(dimethyl)silyl]oxy}-2-(2-chloro-5-methoxyphenyl)-2-azaspiro[4.5]decan-1-one (isomer 1)

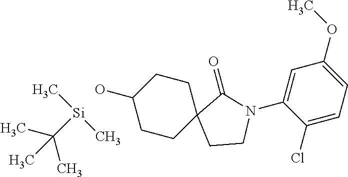

To a mixture of 8-{[tert-butyl(dimethyl)silyl]oxy}-2-azaspiro[4.5]decan-1-one (500 mg, 1.76 mmol) (isomer 1) and 1-chloro-2-iodo-4-methoxybenzene (568 mg, 2.12 mmol) in toluene (38 ml) was added under an argon atmosphere copper(I) iodide (67.2 mg, 353 μmol), N,N′-dimethylethylenediamine (77 μl, 710 μmol) and potassium phosphate (749 mg, 3.53 mmol) and the reaction was heated to 110° C. for 18 hours. Upon cooling, the reaction mixture was filtrated through a pad of celite the residue was washed with ethyl acetate and the filtrate was concentrated under reduced pressure and the crude product was purified by flash chromatography (hexanes/ethyl acetate-gradient, 20%->50% ethyl acetate) to give the title compound as single isomer (564 mg).

LC-MS (Method 2): Rₜ=1.72 min; MS (ESIpos): m/z=424 [M+H]⁺

¹H-NMR (400 MHz, DMSO-d6): δ [ppm]=7.43 (d, 1H), 7.08-6.67 (m, 2H), 3.94 (br s, 1H), 3.76 (s, 3H), 3.60 (t, 2H), 2.15-1.89 (m, 4H), 1.73-1.54 (m, 4H), 1.43-1.27 (m, 2H), 0.93-0.80 (m, 9H), 0.16-0.03 (m, 6H).

### Intermediate I197

### 2-(2-chloro-5-methoxyphenyl)-8-hydroxy-2-azaspiro[4.5]decan-1-one (isomer 1)

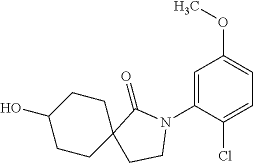 [SMILES: COc1ccc(Cl)c(N2CCC3(CCC(O)CC3)C2=O)c1]

Tetra-N-butylammonium fluoride in THF (3.3 ml, 1.0 M, 3.3 mmol) was added to a solution of 8-{[tert-butyl(dimethyl)silyl]oxy}-2-(2-chloro-5-methoxyphenyl)-2-azaspiro[4.5]decan-1-one (564 mg, 1.33 mmol) (isomer 1) in THF (11 ml) and the mixture was stirred at room temperature for 20 h. Tetra-N-butylammonium fluoride in THF (0.66 ml, 1.0 M, 0.66 mmol) was added to the reaction and the mixture was stirred for 4 hours at room temperature. For work-up the mixture was poured into water, extracted with ethyl acetate (3×) and the combined organic phases were washed with sodium bicarbonate solution and brine, filtrated through a silicone filter and concentrated under reduced pressure. The residue was purified by flash chromatography (25 g Snap cartridge, hexanes/ethyl acetate-gradient, 0%->100% ethyl acetate) to give the title compound (301 mg) as single isomer.

LC-MS (Method 2): Rₜ=0.98 min; MS (ESIpos): m/z=310 [M+H]⁺

¹H-NMR (400 MHz, DMSO-d6) δ[ppm]: 2.010 (1.41), 2.027 (2.71), 2.044 (1.46), 2.075 (16.00), 2.523 (2.13), 3.159 (0.73), 3.171 (0.74), 3.577 (1.61), 3.595 (2.83), 3.612 (1.50), 3.764 (14.27), 4.380 (1.54), 4.388 (1.53), 6.936 (1.04), 6.944 (1.52), 6.958 (0.88), 6.966 (1.97), 6.975 (2.88), 6.983 (1.55), 7.421 (2.34), 7.443 (2.15).

### Intermediate I198

### 2-[2-(2-chloro-5-methoxyphenyl)-1-oxo-2-azaspiro[4.5]dec-8-yl]-1H-isoindole-1,3(2H)-dione (isomer 1)

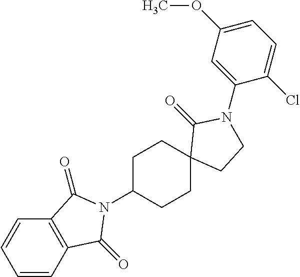 [SMILES: COc1ccc(Cl)c(N2CCC3(CCC(N4C(=O)c5ccccc5C4=O)CC3)C2=O)c1]

Diisopropyl azodicarboxylate (170 μl, 870 μmol) was added drop wise to a mixture of 2-(2-chloro-5-methoxyphenyl)-8-hydroxy-2-azaspiro[4.5]decan-1-one (180 mg, 581 μmol) (isomer 1), 1H-isoindole-1,3(2H)-dione (128 mg, 872 μmol) and triphenylphosphine (229 mg, 872 μmol) in THF (9.4 ml) and the mixture was stirred at room temperature for 48 hours. For work-up, the reaction mixture was concentrated and the residue was purified by flash chromatography (hexanes/ethyl acetate gradient, 20%->100% ethyl acetate to give the title compound (73 mg) as single isomer.

LC-MS (Method 2): Rₜ=1.30 min; MS (ESIpos): m/z=439 [M+H]⁺

¹H-NMR (400 MHz, DMSO-d6) δ[ppm]: 1.065 (0.64), 1.155 (0.77), 1.171 (2.75), 1.186 (2.32), 1.633 (0.68), 1.659 (1.43), 1.667 (1.56), 1.699 (1.45), 1.732 (3.05), 1.756 (2.22), 1.987 (1.19), 2.173 (1.81), 2.191 (3.48), 2.208 (2.01), 2.246 (1.19), 2.254 (1.28), 2.276 (1.11), 2.287 (1.07), 2.327 (1.24), 2.669 (1.15), 3.654 (1.98), 3.671 (3.58), 3.689 (1.86), 3.773 (16.00), 4.017 (0.66), 4.035 (0.87), 4.044 (0.94), 4.053 (0.64), 4.075 (0.43), 6.955 (1.26), 6.962 (1.64), 6.977 (1.28), 6.984 (1.94), 7.008 (3.35), 7.015 (2.37), 7.440 (2.97), 7.462 (2.71), 7.825 (0.96), 7.836 (1.81), 7.848 (7.55), 7.854 (7.85), 7.866 (1.75), 7.877 (0.87), 8.883 (0.47).

### Intermediate I199

### 8-amino-2-(2-chloro-5-methoxyphenyl)-2-azaspiro[4.5]decan-1-one (isomer 1)

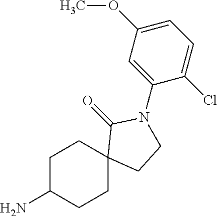 [SMILES: COc1ccc(Cl)c(N2CCC3(CCC(N)CC3)C2=O)c1]

A mixture of 2-[2-(2-chloro-5-methoxyphenyl)-1-oxo-2-azaspiro[4.5]dec-8-yl]-1H-isoindole-1,3(2H)-dione (70.0 mg, 159 μmol) (isomer 1) and hydrazine hydrate (48 μl, 80% purity, 800 μmol) in ethanol (1.4 ml) was stirred at 80° C. for 3 h. Upon cooling, the precipitate was filtrated of and the filtrate was concentrated to give the title compound (43 mg), which was used in the next step without further purification.

### Intermediate I200

### 7-(methoxycarbonyl)-5-methylpyrazolo[1,5-a]pyrimidine-3-carboxylic acid

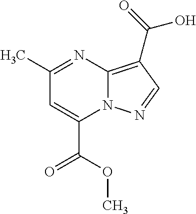 [SMILES: COC(=O)c1cc(C)nc2c(C(=O)O)cnn12]

A mixture of 5-amino-1H-pyrazole-4-carboxylic acid (807 mg, 6.35 mmol) and methyl 2,4-dioxopentanoate (1.83 g, 12.7 mmol) in acetic acid (4.9 ml, 86 mmol) was heated for 1 h at 110° C. in a microwave reactor. Upon cooling to room temperature, the reaction mixture was poured into water and the resulting precipitate was filtered off. The residue was washed with methanol to give the title compound (615 mg, 85% purity) together with unknown impurities.

LC-MS (Method 1): Rₜ=0.62 min; MS (ESIpos): m/z=236 [M+H]⁺

¹H-NMR (400 MHz, DMSO-d6) δ[ppm]: 1.906 (2.11), 2.666 (16.00), 2.855 (1.31), 2.857 (1.32), 3.962 (2.11), 4.003 (15.51), 7.570 (5.55), 8.583 (5.25), 8.753 (0.73).

### Intermediate I201

### methyl 6-chloro-8-[(3,3,3-trifluoropropyl)amino]imidazo[1,2-b]pyridazine-3-carboxylate

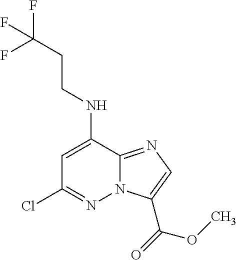 [SMILES: COC(=O)c1cnc2c(NCCC(F)(F)F)cc(Cl)nn12]

A autoclave was charged with a solution of 6-chloro-3-iodo-N-(3,3,3-trifluoropropyl)imidazo[1,2-b]pyridazin-8-amine (3.00 g, 7.68 mmol, described in WO2012032031) in methanol (50 ml) and THF (5.0 ml). [1,1′-Bis(diphenylphosphino)ferrocene]dichloropalladium(II), complex with dichloromethane (1.25 g, 1.54 mmol) and triethylamine (1.2 ml, 8.4 mmol) were added and the mixture was purged with carbon monoxide (3×). The reaction was pressurized with carbon monoxide (10.6 bar) and was stirred at room temperature and for 18 hours at 100° C. (14.4 bar). For work-up the mixture was concentrated under reduced pressure to give the title product.

LC-MS (Method 1): Rₜ=1.10 min; MS (ESIpos): m/z=323 [M+H]⁺

### Intermediate I202

### 6-chloro-8-[(3,3,3-trifluoropropyl)amino]imidazo[1,2-b]pyridazine-3-carboxylic acid

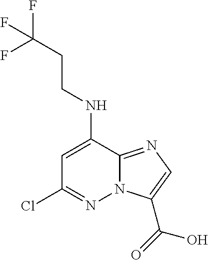 [SMILES: O=C(O)c1cnc2c(NCCC(F)(F)F)cc(Cl)nn12]

To a solution of methyl 6-chloro-8-[(3,3,3-trifluoropropyl)amino]imidazo[1,2-b]pyridazine-3-carboxylate (217 mg, 673 μmol) in methanol (1.1 ml) and THF (3.8 ml) was added a solution of lithium hydroxide in water (1.7 ml, 1.0 M, 1.7 mmol). The reaction was stirred for 3 days at room temperature. For workup the reaction was concentrated and residue was dissolved in water. Citric acid was added until a weak acidic pH was reached and the mixture was extracted with dichloromethane/2-propanol (4:1). The combined organic phases were filtrated over a hydrophobic filter and concentrated to give the title product (87 mg).

LC-MS (Method 1): Rₜ=0.92 min; MS (ESIpos): m/z=309 [M+H]⁺

### Intermediate I203

### 5,7-diethylpyrazolo[1,5-a]pyrimidine-3-carboxylic acid

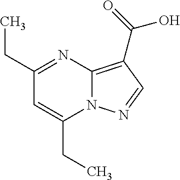 [SMILES: CCc1cc(CC)n2ncc(C(=O)O)c2n1]

A mixture of 5-amino-1H-pyrazole-4-carboxylic acid (1.00 g, 7.87 mmol) and heptane-3,5-dione (2.02 g, 15.7 mmol) in acetic acid (7.4 ml) was heated for 1 h at 110° C. in a microwave reactor. Upon cooling to room temperature, the reaction mixture portioned between water and dichloromethane and the organic phase was washed with water, filtrated through a silicone filter and concentrated under vacuum. The crude product was triturated with ethyl acetate to give the title product (948 mg).

LC-MS (Method 1): Rₜ=0.84 min; MS (ESIpos): m/z=220 [M+H]⁺

¹H-NMR (400 MHz, DMSO-d6) δ [ppm]: 1.275 (6.90), 1.294 (16.00), 1.313 (6.78), 1.332 (6.44), 1.351 (13.58), 1.369 (6.57), 2.842 (1.89), 2.861 (5.72), 2.880 (5.54), 2.899 (1.71), 3.108 (1.17), 3.110 (1.18), 3.126 (3.58), 3.128 (3.68), 3.145 (3.31), 3.147 (3.47), 3.164 (1.08), 3.166 (1.11), 7.116 (5.07), 8.515 (8.35).

### Intermediate I204

### 8-{[tert-butyl(dimethyl)silyl]oxy}-2-(pyridin-2-yl)-2-azaspiro[4.5]decan-1-one (isomer 1)

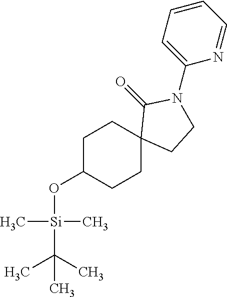 [SMILES: CC(C)(C)[Si](C)(C)OC1CCC2(CC1)CCN(c1ccccn1)C2=O]

To a mixture of 8-{[tert-butyl(dimethyl)silyl]oxy}-2-azaspiro[4.5]decan-1-one (593 mg, 2.09 mmol) (isomer 1) and 2-iodopyridine (515 mg, 2.51 mmol) in toluene (14 ml) was added under an argon atmosphere copper(I) iodide (79.7 mg, 418 μmol), N,N′-dimethylethylenediamine (91 μl, 840 μmol) and potassium phosphate (888 mg, 4.18 mmol) and the reaction was heated to 80° C. for 18 h in a microwave reactor. Upon cooling, the reaction mixture was filtrated through a pad of celite and the filtrate was concentrated under reduced pressure and the crude product was purified by flash chromatography (hexanes/ethyl acetate, 4:1) to give the title compound as single isomer (751 mg).

LC-MS (Method 2): Rₜ=1.72 min; MS (ESIpos): m/z=361 [M+H]⁺

¹H-NMR (400 MHz, DMSO-d6) δ[ppm]: 0.843 (1.01), 0.850 (16.00), 1.924 (0.84), 1.941 (1.09), 3.869 (0.93), 8.263 (0.77), 8.283 (0.65).

### Intermediate I205

### 8-hydroxy-2-(pyridin-2-yl)-2-azaspiro[4.5]decan-1-one (isomer 1)

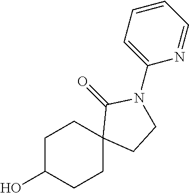 [SMILES: O=C1N(c2ccccn2)CCC12CCC(O)CC2]

Tetra-N-butylammonium fluoride in THF (5.2 ml, 1.0 M, 5.2 mmol) was added to a solution of 8-{[tert-butyl(dimethyl)silyl]oxy}-2-(pyridin-2-yl)-2-azaspiro[4.5]decan-1-one (746 mg, 2.07 mmol) (isomer 1) in THF (18 ml) and the mixture was stirred at room temperature for 20 h. The mixture was poured into water, extracted with ethyl acetate (3×) and the combined organic phases were washed with sodium bicarbonate solution and brine, filtrated through a silicone filter and concentrated under reduced pressure. The residue was purified by flash chromatography (hexanes/ethyl acetate-gradient, 50%->100% ethyl acetate) to give the title compound (421 mg) as single isomer.

LC-MS (Method 2): Rₜ=0.82 min; MS (ESIpos): m/z=247 [M+H]⁺

¹H-NMR (400 MHz, DMSO-d₆): δ [ppm]=8.38 (ddd, 1H), 8.32 (dt, 1H), 7.87-7.59 (m, 1H), 7.13 (ddd, 1H), 4.42 (d, 1H), 3.91 (t, 2H), 3.77 (br d, 1H), 2.07-1.86 (m, 4H), 1.74-1.61 (m, 2H), 1.60-1.48 (m, 2H), 1.31-1.18 (m, 2H).

### Intermediate I206

### 2-[1-oxo-2-(pyridin-2-yl)-2-azaspiro[4.5]dec-8-yl]-1H-isoindole-1,3(2H)-dione (isomer 1)

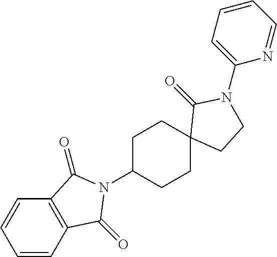 [SMILES: O=C1c2ccccc2C(=O)N1C1CCC2(CC1)CCN(c1ccccn1)C2=O]

Diisopropyl azodicarboxylate (500 μl, 2.6 mmol) was added drop wise to a mixture of 8-hydroxy-2-(pyridin-2-yl)-2-azaspiro[4.5]decan-1-one (419 mg, 1.70 mmol) (isomer 1), 1H-isoindole-1,3(2H)-dione (375 mg, 2.55 mmol) and triphenylphosphine (669 mg, 2.55 mmol) in THF (28 ml) and the mixture was stirred at room temperature for 12 hours. For work-up, the reaction mixture was concentrated and the residue was purified by flash chromatography (hexanes/ethyl acetate gradient, 20%->50% ethyl acetate to give the title compound (551 mg) as single isomer.

LC-MS (Method 2): Rₜ=1.20 min; MS (ESIpos): m/z=376 [M+H]⁺

### Intermediate I207

### 8-amino-2-(pyridin-2-yl)-2-azaspiro[4.5]decan-1-one (isomer 1)

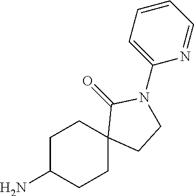 [SMILES: NC1CCC2(CC1)CCN(c1ccccn1)C2=O]

A mixture of 2-[1-oxo-2-(pyridin-2-yl)-2-azaspiro[4.5]dec-8-yl]-1H-isoindole-1,3(2H)-dione (548 mg, 1.46 mmol) (isomer 1) and hydrazine hydrate (440 μl, 80% purity, 7.3 mmol) in ethanol (12 ml) was stirred at 80° C. for 3 h. Upon cooling, the precipitate was filtrated off, washed with ethanol and the filtrate was concentrated and the residue was purified by flash chromatography (dichloromethane/methanol 9:1) to give the title compound (151 mg).

LC-MS (Method 2): Rₜ=0.81 min; MS (ESIpos): m/z=246 [M+H]⁺

¹H-NMR (400 MHz, DMSO-d6) δ[ppm]: 1.065 (1.30), 1.079 (1.35), 1.094 (3.29), 1.108 (3.59), 1.125 (3.90), 1.141 (3.59), 1.154 (2.10), 1.170 (3.76), 1.186 (2.43), 1.486 (2.25), 1.515 (2.34), 1.524 (2.74), 1.534 (13.35), 1.543 (16.00), 1.555 (5.88), 1.564 (6.00), 1.572 (6.19), 1.597 (1.16), 1.605 (1.28), 1.688 (4.42), 1.697 (4.44), 1.709 (2.46), 1.721 (4.16), 1.731 (3.76), 1.962 (9.12), 1.974 (3.29), 1.979 (12.74), 1.984 (3.31), 1.997 (9.45), 2.323 (0.99), 2.327 (1.37), 2.331 (0.97), 2.518 (7.78), 2.523 (4.06), 2.529 (2.32), 2.537 (1.28), 2.547 (1.77), 2.557 (0.85), 2.665 (1.02), 2.669 (1.39), 2.674 (0.97), 3.160 (1.06), 3.170 (1.11), 3.900 (10.54), 3.912 (3.31), 3.918 (13.14), 3.923 (3.64), 3.936 (10.02), 7.115 (4.25), 7.117 (4.37), 7.127 (4.61), 7.129 (5.13), 7.132 (5.08), 7.135 (4.80), 7.145 (4.77), 7.148 (4.58), 7.776 (4.06), 7.781 (4.40), 7.794 (4.44), 7.797 (5.44), 7.799 (5.18), 7.802 (4.66), 7.815 (3.95), 7.820 (4.28), 8.295 (5.22), 8.297 (9.97), 8.300 (5.72), 8.316 (4.92), 8.318 (8.63), 8.321 (5.36), 8.372 (4.61), 8.374 (5.27), 8.377 (5.62), 8.379 (4.66), 8.384 (4.87), 8.386 (5.29), 8.389 (4.99), 8.391 (4.54).

### Intermediate I208

### phenyl 5-{[2-(2-chloro-4-fluorophenyl)-1-oxo-2-azaspiro[4.5]dec-8-yl]carbamoyl}-1H-imidazole-4-carboxylate (isomer 2)

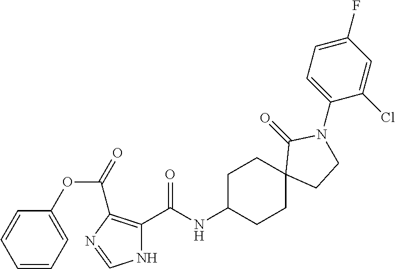 [SMILES: O=C(Oc1ccccc1)c1nc[nH]c1C(=O)NC1CCC2(CC1)CCN(c1ccc(F)cc1Cl)C2=O]

To a suspension of 8-amino-2-(2-chloro-4-fluorophenyl)-2-azaspiro[4.5]decan-1-one (730 mg, 2.46 mmol) (isomer 2) and diphenyl 5,10-dioxo-5H,10H-diimidazo[1,5-a:1′,5′-d]pyrazine-1,6-dicarboxylate (632 mg, 1.48 mmol) in THF (38 ml) was added triethylamine (410 μl, 3.0 mmol) and the mixture was stirred for 5 h at room temperature. The reaction mixture was filtrated and the filtrate was concentrated under reduced pressure to give the title compound as a solid material (1.14 g, isomer 2)

LC-MS (Method 1): Rₜ=1.18 min; MS (ESIpos): m/z=511 [M+H]⁺

### Intermediate I209

### ethyl 5-[4-(tert-butoxycarbonyl)piperazin-1-yl]pyrazolo[1,5-a]pyrimidine-3-carboxylate

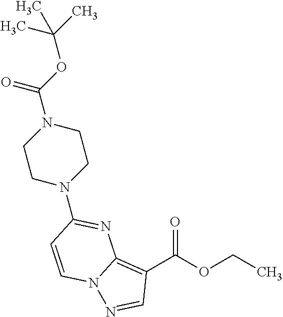 [SMILES: CCOC(=O)c1cnn2ccc(N3CCN(C(=O)OC(C)(C)C)CC3)nc12]

A mixture of ethyl 5-chloropyrazolo[1,5-a]pyrimidine-3-carboxylate (677 mg, 3.00 mmol), tert-butyl piperazine-1-carboxylate (838 mg, 4.50 mmol) and N,N-diisopropylethylamine (1.6 ml, 9.0 mmol) in 2-propanol (20 ml) was refluxed for 5 h. Upon cooling, ice water was added and the mixture was extracted with ethyl acetate (3×). The combined organic phases were filtrated through a silicone filter and concentrated to give the title compound.

LC-MS (Method 1): Rₜ=1.11 min; MS (ESIpos): m/z=376 [M+H]⁺

### Intermediate I210

### 5-[4-(tert-butoxycarbonyl)piperazin-1-yl]pyrazolo[1,5-a]pyrimidine-3-carboxylic acid

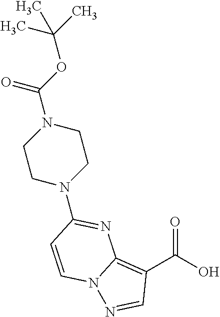 [SMILES: CC(C)(C)OC(=O)N1CCN(c2ccn3ncc(C(=O)O)c3n2)CC1]

To a mixture of ethyl 5-[4-(tert-butoxycarbonyl)piperazin-1-yl]pyrazolo[1,5-a]pyrimidine-3-carboxylate (1.28 g, 3.41 mmol) in THF (23 ml) and methanol (6.8 ml) was added a solution of sodium hydroxide (4.3 ml, 2.0 M, 8.5 mmol), and the reaction was stirred for 3 h at 70° C. Upon cooling, the reaction mixture was concentrated and residue was dissolved in water. Citric acid was added until a weak acidic pH was reached. The resulting precipitate was filtrated of and the residue was washed with water to give the title compound (658 mg) which was used in the next step without further purification.

LC-MS (Method 2): Rₜ=0.46 min; MS (ESIneg): m/z=346 [M−H]⁻

¹H-NMR (400 MHz, DMSO-d6) δ[ppm]: 1.396 (0.70), 1.428 (16.00), 3.449 (1.26), 3.462 (0.98), 3.758 (1.07), 6.829 (0.91), 6.849 (0.92), 8.186 (2.54), 8.741 (1.21), 8.761 (1.18).

### Intermediate I211

### 5-methoxypyrazolo[1,5-a]pyrimidine-3-carboxylic acid

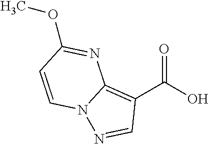

A mixture of ethyl 5-chloropyrazolo[1,5-a]pyrimidine-3-carboxylate (677 mg, 3.00 mmol) and sodium methylate (24 ml, 0.50 M, 12 mmol) was stirred under reflux for 5 hours. A solution of sodium hydroxide (3.0 ml, 2.0 M, 6.0 mmol) was added and the reaction was stirred at room temperature for 12 hours. Upon cooling, the reaction mixture was filtrated and the filtrate was concentrated under reduced pressure. The residue was dissolved in water. Citric acid was added until a weak acidic pH was reached. The resulting precipitate was filtrated of and the residue was washed with water to give the title compound (172 mg) which was used in the next step without further purification.

¹H-NMR (400 MHz, DMSO-d6) δ[ppm]: 2.518 (1.24), 2.522 (1.06), 4.002 (16.00), 4.019 (3.29), 6.717 (2.50), 6.736 (2.61), 6.756 (0.50), 6.774 (0.49), 8.366 (4.45), 8.428 (0.79), 8.987 (3.11), 9.005 (3.46), 9.016 (0.71), 9.035 (0.58).

### Intermediate I212

### ethyl 5-(dimethylamino)pyrazolo[1,5-a]pyrimidine-3-carboxylate

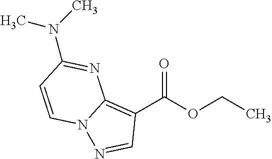 [SMILES: CCOC(=O)c1cnn2ccc(N(C)C)nc12]

A mixture of ethyl 5-chloropyrazolo[1,5-a]pyrimidine-3-carboxylate (677 mg, 3.00 mmol), N-methylmethanamine hydrochloride (489 mg, 6.00 mmol) and N,N-diisopropylethylamine (1.6 ml, 9.0 mmol) in 2-propanol (20 ml) was refluxed for 5 h. Upon cooling, ice water was added and the mixture was extracted with ethyl acetate (3×). The combined organic phases were filtrated through a silicone filter and concentrated to give the title compound (674 mg).

LC-MS (Method 1): Rₜ=0.84 min; MS (ESIpos): m/z=235 [M+H]⁺

¹H-NMR (400 MHz, DMSO-d6) δ[ppm]: 1.261 (7.26), 1.278 (16.00), 1.296 (7.57), 3.182 (14.34), 4.157 (2.25), 4.175 (7.19), 4.193 (7.07), 4.211 (2.14), 6.691 (4.60), 6.710 (4.61), 8.181 (9.13), 8.673 (5.48), 8.693 (5.36).

### Intermediate I213

### 5-(dimethylamino)pyrazolo[1,5-a]pyrimidine-3-carboxylic acid

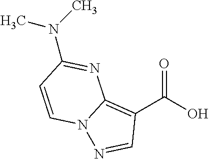

To a mixture of ethyl 5-(dimethylamino)pyrazolo[1,5-a]pyrimidine-3-carboxylate (674 mg, 2.88 mmol) in THF (19 ml) and methanol (5.8 ml) was added a solution of lithium hydroxide (5.8 ml, 1.0 M, 5.8 mmol), and the reaction was stirred for 18 h at room temperature. A solution of sodium hydroxide (2.9 ml, 2.0 M, 5.8 mmol) was added and the mixture was stirred for 24 hours at room temperature and for 3 hours at 70° C. Upon cooling, the reaction mixture was concentrated and residue was dissolved in water. Citric acid was added until a weak acidic pH was reached. The resulting precipitate was filtrated of and the residue was washed with water to give the title compound (520 mg) which was used in the next step without further purification.

LC-MS (Method 1): Rₜ=0.63 min; MS (ESIpos): m/z=207 [M+H]⁺

¹H-NMR (400 MHz, DMSO-d6) δ[ppm]: 3.177 (16.00), 6.683 (3.22), 6.703 (3.24), 8.158 (6.06), 8.672 (3.59), 8.692 (3.45).

### Intermediate I214

### ethyl 5-(methylamino)pyrazolo[1,5-a]pyrimidine-3-carboxylate

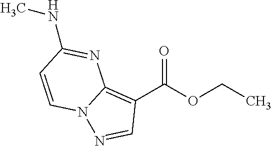 [SMILES: CCOC(=O)c1cnn2ccc(NC)nc12]

A mixture of ethyl 5-chloropyrazolo[1,5-a]pyrimidine-3-carboxylate (677 mg, 3.00 mmol), methanamine (3.0 ml, 2.0 M, 6.0 mmol) and N,N-diisopropylethylamine (1.6 ml, 9.0 mmol) in 2-propanol (20 ml) was refluxed for 5 h. Upon cooling, ice water was added and the mixture was extracted with ethyl acetate (3×). The combined organic phases were filtrated through a silicone filter and concentrated to give the title compound (570 mg).

LC-MS (Method 1): Rₜ=0.74 min; MS (ESIneg): m/z=219 [M−H]⁻

¹H-NMR (400 MHz, DMSO-d6) δ[ppm]: 1.258 (7.44), 1.276 (16.00), 1.293 (7.62), 1.296 (2.96), 1.338 (1.13), 2.902 (6.89), 2.914 (6.83), 4.155 (3.10), 4.173 (9.99), 4.191 (9.82), 4.209 (2.93), 6.336 (1.70), 6.355 (1.74), 7.855 (1.41), 7.867 (1.40), 8.126 (5.66), 8.482 (1.60), 8.501 (1.56).

### Intermediate I215

### 5-(methylamino)pyrazolo[1,5-a]pyrimidine-3-carboxylic acid

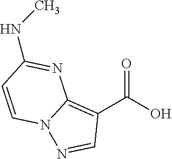

To a mixture ethyl 5-(methylamino)pyrazolo[1,5-a]pyrimidine-3-carboxylate (565 mg, 2.57 mmol) in THF (17 ml) and methanol (5.1 ml) was added a solution of sodium hydroxide (3.2 ml, 2.0 M, 6.4 mmol), and the reaction was stirred for 18 h at room temperature. A solution of sodium hydroxide (2.9 ml, 2.0 M, 5.8 mmol) was added and the mixture was stirred for 3 hours at 70° C. Upon cooling, the reaction mixture was concentrated and residue was dissolved in water. Citric acid was added until a weak acidic pH was reached. The resulting precipitate was filtrated of and the residue was washed with water to give the title compound (483 mg) which was used in the next step without further purification.

LC-MS (Method 1): Rₜ=0.50 min; MS (ESIpos): m/z=193 [M+H]⁺

¹H-NMR (400 MHz, DMSO-d6) δ[ppm]: 1.907 (0.74), 2.322 (1.34), 2.327 (1.81), 2.332 (1.37), 2.518 (4.64), 2.523 (3.49), 2.665 (1.21), 2.669 (1.67), 2.673 (1.15), 2.891 (16.00), 2.903 (15.48), 6.332 (3.40), 6.351 (3.40), 7.879 (3.54), 7.891 (3.40), 8.109 (13.67), 8.488 (3.57), 8.506 (3.02), 8.735 (1.34), 11.638 (1.04).

### Intermediate I216

### methyl 1-fluoro-4-({[5-(methylcarbamoyl)-1H-imidazol-4-yl]carbonyl}amino)cyclohexanecarboxylate

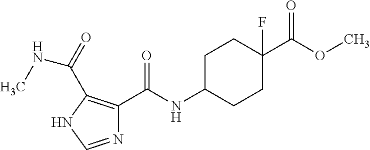

To a suspension of 5,10-dioxo-5H,10H-diimidazo[1,5-a:1′,5′-d]pyrazine-1,6-dicarbonyl dichloride (313 mg, 1.00 mmol) in THF (8 ml) was added methyl 4-amino-1-fluorocyclohexanecarboxylate (350 mg, 2.00 mmol) and N,N-diisopropylethylamine (870 μl, 5.0 mmol) and the mixture was stirred at room temperature for 6 hours. A solution of methanamine (1.0 ml, 2.0 M, 2.0 mmol) and N,N-diisopropylethylamine (520 μl, 3.0 mmol) in THF (8 ml) was added and the mixture was stirred for 18 hours at room temperature. For work-up the reaction was filtrated and the filtrate was concentrated under reduced pressure. The residue was purified by flash chromatography (hexane/ethyl acetate gradient, 50->100% ethyl acetate) to give the title compound (298 mg).

LC-MS (Method 1): Rₜ=0.83 min; MS (ESIpos): m/z=327 [M+H]⁺

¹H-NMR (400 MHz, DMSO-d6) δ[ppm]: 1.829 (1.12), 1.857 (2.09), 1.891 (1.08), 1.987 (0.79), 2.523 (1.93), 2.800 (5.67), 2.813 (5.68), 3.622 (0.80), 3.739 (16.00), 7.810 (6.68).

### Intermediate I217

### 1-fluoro-4-({[5-(methylcarbamoyl)-1H-imidazol-4-yl]carbonyl}amino)cyclohexanecarboxylic acid

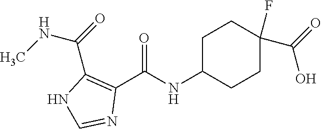 [SMILES: CNC(=O)c1[nH]cnc1C(=O)NC1CCC(F)(C(=O)O)CC1]

Lithium hydroxide (2.2 ml, 1.0 M, 2.2 mmol) was added to a solution of methyl 1-fluoro-4-({[5-(methylcarbamoyl)-1H-imidazol-4-yl]carbonyl}amino)cyclohexanecarboxylate (290 mg, 889 μmol) in THF (5.8 ml) and methanol (1.5 ml) and the mixture was stirred at room temperature for 18 hours. For work-up, the mixture was concentrated, then poured into water and acidified with hydrochloric acid and the mixture was extracted with dichloromethane/2-propanol. The combined organic phases were filtrated through a hydrophobic filter and concentrated to give the title product (217 mg).

LC-MS (Method 1): Rₜ=0.66 min; MS (ESIpos): m/z=313 [M+H]⁺

¹H-NMR (400 MHz, DMSO-d6) δ[ppm]: 1.026 (15.84), 1.041 (16.00), 1.740 (2.02), 1.831 (3.60), 1.854 (3.67), 1.907 (1.01), 2.084 (1.82), 2.523 (3.98), 2.784 (10.74), 2.797 (11.03), 2.831 (1.41), 7.808 (6.79), 7.811 (8.40), 8.623 (1.58), 8.635 (1.61), 11.478 (1.88), 11.495 (1.89), 13.226 (1.84).

### Intermediate I218

### methyl 1-fluoro-4-[(pyrazolo[1,5-a]pyrimidin-3-ylcarbonyl)amino]cyclohexanecarboxylate

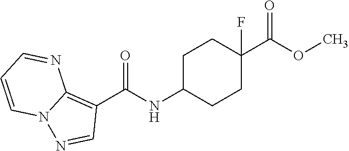 [SMILES: COC(=O)C1(F)CCC(NC(=O)c2cnn3cccnc23)CC1]

To a solution of pyrazolo[1,5-a]pyrimidine-3-carboxylic acid (163 mg, 1.00 mmol) and methyl 4-amino-1-fluorocyclohexanecarboxylate (193 mg) in DMF (11 ml) was added PyBOP (572 mg, 1.10 mmol) and N,N-diisopropylethylamine (700 μl, 4.0 mmol) and the reaction was stirred for 12 h at room temperature. For work-up, the reaction was concentrated under reduced pressure and the residue was purified by flash chromatography (hexane/ethyl acetate gradient, 50->100% ethyl acetate) to give the title compound (294 mg).

LC-MS (Method 1): Rₜ=0.84 min; MS (ESIpos): m/z=321 [M+H]⁺

¹H-NMR (400 MHz, DMSO-d6) δ[ppm]: 1.154 (4.34), 1.172 (9.22), 1.190 (4.65), 1.747 (1.39), 1.907 (2.49), 1.914 (1.70), 1.988 (16.00), 3.631 (0.68), 3.759 (14.93), 3.999 (1.23), 4.017 (3.72), 4.035 (3.69), 4.053 (1.19), 7.263 (1.55), 7.273 (1.52), 7.280 (1.55), 7.291 (1.56), 8.586 (5.15), 8.815 (1.76), 8.819 (1.92), 8.826 (1.83), 8.829 (1.61), 9.319 (1.83), 9.323 (1.73), 9.337 (1.76), 9.340 (1.71).

### Intermediate I219

### 1-fluoro-4-[(pyrazolo[1,5-a]pyrimidin-3-ylcarbonyl)amino]cyclohexanecarboxylic acid

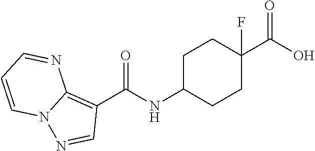 [SMILES: O=C(NC1CCC(F)(C(=O)O)CC1)c1cnn2cccnc12]

Lithium hydroxide (2.3 ml, 1.0 M, 2.3 mmol) was added to a solution of methyl 1-fluoro-4-[(pyrazolo[1,5-a]pyrimidin-3-ylcarbonyl)amino]cyclohexanecarboxylate (290 mg, 905 μmol) in THF (5.9 ml) and methanol (1.6 ml) and the mixture was stirred at room temperature for 18 hours. For work-up, the mixture was concentrated, then poured into water and acidified with hydrochloric acid the resulting precipitate was filtrated off and was washed with water and ethanol to give the title product (149 mg).

LC-MS (Method 1): Rₜ=0.67 min; MS (ESIpos): m/z=307 [M+H]⁺

¹H-NMR (400 MHz, DMSO-d6) δ[ppm]: 1.754 (2.41), 1.874 (3.99), 1.898 (4.38), 1.907 (3.41), 2.043 (1.24), 2.077 (1.55), 2.116 (1.43), 2.145 (1.66), 2.523 (3.32), 4.145 (1.52), 7.257 (4.28), 7.268 (4.50), 7.275 (4.45), 7.286 (4.42), 8.097 (2.57), 8.116 (2.58), 8.582 (16.00), 8.793 (5.11), 8.797 (5.11), 8.804 (5.03), 8.808 (5.11), 9.314 (5.03), 9.318 (5.50), 9.331 (5.51), 9.336 (4.82).

### 4. Examples

### Example 1

### N⁵-{trans-4-[(2-chloro-4-fluorophenyl)carbamoyl]cyclohexyl}-N⁴-methyl-1H-imidazole-4,5-dicarboxamide

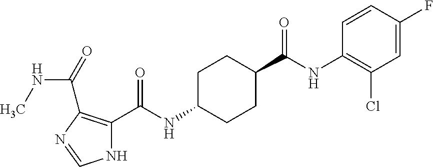 [SMILES: CNC(=O)c1nc[nH]c1C(=O)N[C@H]1CC[C@H](C(=O)Nc2ccc(F)cc2Cl)CC1]

### Step 1:

To a suspension of 500 mg (1.40 mmol) trans-4-({[4-(phenoxycarbonyl)-1H-imidazol-5-yl]carbonyl}amino)cyclohexane-carboxylic acid in 25 ml dichloromethane 312 μl (2.24 mmol) 1-chloro-N,N,2-trimethylprop-1-en-1-amine were added and the mixture was stirred at room temperature for 30 minutes. 339 μl (4.20 mmol) pyridine and 167 μl (1.40 mmol) 2-chloro-4-fluoroaniline were added and the reaction was stirred at room temperature for 30 minutes.

### Step 2:

To the crude reaction mixture were added 3.5 ml (2 M in tetrahydrofuran, 7 mmol) methanamine and the mixture was stirred for 2 days at room temperature. The reaction mixture was diluted with 20 ml tetrahydrofuran and the resulting precipitate was filtered off and was washed with dichloromethane. The solid was dissolved in 50 ml tetrahydrofuran and a few drops of water. 200 ml water were added and the mixture was stirred for 30 minutes. The precipitate was filtered off to give after trituration with diethyl ether and drying under vacuum at 50° C. 166 mg of the title compound as a solid material.

LC-MS (Method 2): Rₜ=0.98 min; MS (ESIpos) m/z=422.2 [M+H]⁺.

¹H-NMR (400 MHz, DMSO-d₆), δ [ppm]=13.17 (br. s., 1H), 11.31-10.88 (m, 1H), 9.47 (s, 1H), 8.73-8.26 (m, 1H), 7.79 (s, 1H), 7.59 (dd, 1H), 7.48 (dd, 1H), 7.20 (td, 1H), 3.72 (br. s., 1H), 2.81 (br. s., 3H), 2.14-1.80 (m, 4H), 1.66-1.16 (m, 5H).

### Example 2

### N⁵-{trans-4-[(2-chloro-4-fluorophenyl)carbamoyl]cyclohexyl}-N⁴-[2-(piperidin-1-yl)ethyl]-1H-imidazole-4,5-dicarboxamide

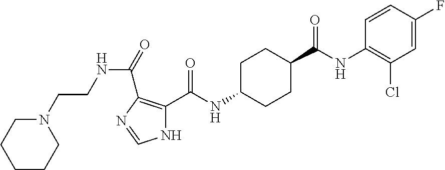 [SMILES: O=C(NCCN1CCCCC1)c1nc[nH]c1C(=O)N[C@H]1CC[C@H](C(=O)Nc2ccc(F)cc2Cl)CC1]

### Step 1:

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 33 μl (0.28 mmol) 2-chloro-4-fluoroaniline were added and the reaction was stirred at room temperature for 30 minutes.

### Step 2

To the crude reaction mixture were added 5 ml tetrahydrofuran and 200 μl (1.40 mmol) 2-(piperidin-1-yl)ethanamine and the mixture was stirred for 3 hours at room temperature. The dichloromethane was distilled of and the residue was diluted with tetrahydrofuran and water. The resulting precipitate was filtered off and was washed with water. The crude product was purified by flash column chromatography (dichloromethane/methanol-gradient) to give 45 mg (contains 6% DCM) of the title compound as a solid material.

LC-MS (Method 2): Rₜ=1.18 min; MS (ESIpos) m/z=519.2 [M+H]⁺.

¹H-NMR (400 MHz, DMSO-d₆), δ [ppm]=13.20 (br. s., 1H), 11.16 (br. s., 1H), 9.49 (br. s., 1H), 8.64-8.17 (m, 1H), 7.81 (s, 1H), 7.61 (dd, 1H), 7.50 (dd, 1H), 7.22 (td, 1H), 5.76 (s, 1H), 3.73 (br. s., 1H), 3.40 (q, 2H), 2.48-2.35 (m, 6H), 2.13-1.79 (m, 4H), 1.66-1.20 (m, 10H).

### Example 3

### N⁵-{trans-4-[(2-chloro-4,5-difluorophenyl)carbamoyl]cyclohexyl}-N⁴-methyl-1H-imidazole-4,5-dicarboxamide

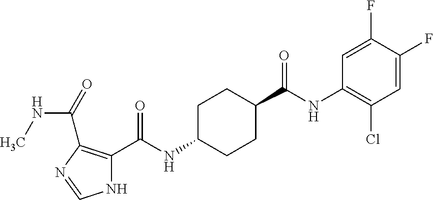 [SMILES: CNC(=O)c1nc[nH]c1C(=O)N[C@H]1CC[C@H](C(=O)Nc2cc(F)c(F)cc2Cl)CC1]

### Step 1:

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 31 μl (0.28 mmol) 2-chloro-4,5-difluoroaniline were added and the reaction was stirred at room temperature for 30 minutes.

### Step 2

To the crude reaction mixture were added 5 ml tetrahydrofuran and 700 μl (2 M in tetrahydrofuran, 1.40 mmol) methanamine and the mixture was stirred for 2.5 hours at 50° C. The reaction mixture was concentrated in vacuo and the residue was dissolved in tetrahydrofuran. Water was added and the resulting precipitate was filtered off and was washed with water. The crude product was purified by flash column chromatography (dichloromethane/methanol-gradient) to give 55 mg (contains 3.5% DCM) of the title compound as a solid material.

LC-MS (Method 2): Rₜ=1.04 min; MS (ESIpos) m/z=440.0 [M+H]⁺.

¹H-NMR (400 MHz, DMSO-d₆), δ [ppm]=13.17 (br. s., 1H), 11.55-10.74 (m, 1H), 9.56 (s, 1H), 8.82-8.26 (m, 1H), 7.95-7.59 (m, 3H), 5.74 (s, 1H), 3.72 (br. s., 1H), 2.81 (d, 3H), 2.15-1.82 (m, 4H), 1.68-1.15 (m, 4H).

### Example 4

### N⁵-{trans-4-[(2-chloro-4,5-difluorophenyl)carbamoyl]cyclohexyl}-N⁴-[2-(piperidin-1-yl)ethyl]-1H-imidazole-4,5-dicarboxamide

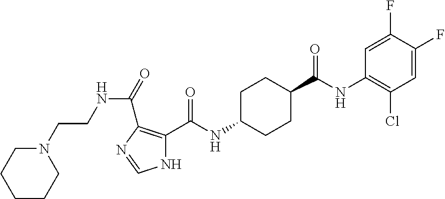 [SMILES: O=C(NCCN1CCCCC1)c1nc[nH]c1C(=O)N[C@H]1CC[C@H](C(=O)Nc2cc(F)c(F)cc2Cl)CC1]

### Step 1:

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 31 μl (0.28 mmol) 2-chloro-4,5-difluoroaniline were added and the reaction was stirred at room temperature for 30 minutes.

### Step 2

To the crude reaction mixture were added 5 ml tetrahydrofuran and 200 μl (1.40 mmol) 2-(piperidin-1-yl)ethanamine and the mixture was stirred for 2.5 hours at 50° C. The reaction mixture was concentrated in vacuo and the residue was dissolved in tetrahydrofuran. Water was added and the resulting precipitate was filtered off and was washed with water. The crude product was triturated with diisopropyl ether/acetone to give after washing with diisopropyl ether and drying under vacuum at 55° C. 64 mg (contains 7% tetrahydrofuran) of the title compound as a solid material.

LC-MS (Method 2): Rₜ=1.25 min; MS (ESIpos) m/z=537.2 [M+H]⁺.

¹H-NMR (400 MHz, DMSO-d₆), δ [ppm]=13.18 (br. s., 1H), 11.13 (br. s., 1H), 9.56 (br. s., 1H), 8.41 (br. s., 1H), 7.93-7.60 (m, 3H), 3.72 (br. s., 1H), 3.59 (s, 1H), 3.38 (q, 2H), 2.45-2.29 (m, 6H), 2.12-1.85 (m, 4H), 1.75 (s, 1H), 1.64-1.20 (m, 11H).

### Example 5

### N⁵-{trans-4-[(2-chloro-4,5-dimethoxyphenyl)carbamoyl]cyclohexyl}-N⁴-methyl-1H-imidazole-4,5-dicarboxamide

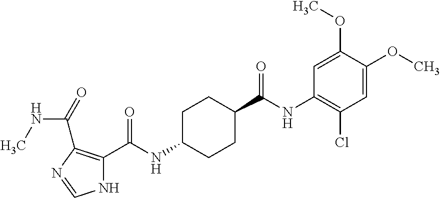

Step 1:

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 42 μl (0.28 mmol) 2-chloro-4,5-dimethoxyaniline were added and the reaction was stirred at room temperature for 30 minutes.

LC-MS (Method 2): Rₜ=0.90 min; MS (ESIpos) m/z=527.1 [M+H]⁺.

### Step 2

To the crude reaction mixture were added 5 ml tetrahydrofuran and 700 μl (2 M in tetrahydrofuran, 1.40 mmol) methanamine and the mixture was stirred for 2.5 hours at 50° C.

The reaction mixture was concentrated in vacuo and the residue was dissolved in tetrahydrofuran. Water was added and the resulting precipitate was filtered off and was washed with water. The crude product was triturated with dichloromethane to give after drying under vacuum at 55° C. 62 mg of the title compound as a solid material.

LC-MS (Method 2): Rₜ=0.89 min; MS (ESIpos) m/z=464.1 [M+H]⁺.

¹H-NMR (400 MHz, DMSO-d₆), δ [ppm]=13.13 (br. s., 1H), 11.34-10.94 (m, 1H), 9.28 (br. s., 1H), 8.68-8.27 (m, 1H), 7.79 (s, 1H), 7.17 (s, 1H), 7.03 (s, 1H), 3.75 (s, 3H), 3.72 (s, 3H), 2.80 (br. s., 3H), 2.14-1.82 (m, 4H), 1.69-1.17 (m, 6H).

### Example 6

### N⁵-{trans-4-[(2-chloro-4,6-difluorophenyl)carbamoyl]cyclohexyl}-N⁴-methyl-1H-imidazole-4,5-dicarboxamide

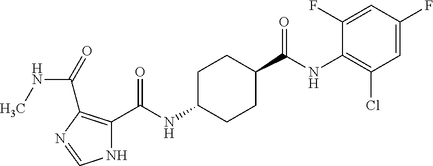 [SMILES: CNC(=O)c1nc[nH]c1C(=O)N[C@H]1CC[C@H](C(=O)Nc2c(F)cc(F)cc2Cl)CC1]

### Step 1:

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 31 μl (0.28 mmol) 2-chloro-4,6-difluoroaniline were added and the reaction was stirred at room temperature for 30 minutes.

### Step 2

To the crude reaction mixture were added 5 ml tetrahydrofuran and 700 μl (2 M in tetrahydrofuran, 1.40 mmol) methanamine and the mixture was stirred for 2.5 hours at 50° C. The reaction mixture was concentrated in vacuo and the residue was dissolved in tetrahydrofuran. Water was added and the resulting precipitate was filtered off and was washed with water. The crude product was triturated with water and DMSO to give after drying under vacuum at 55° C. 50 mg of the title compound as a solid material.

LC-MS (Method 6): Rₜ=0.87 min; MS (ESIpos) m/z=440.1 [M+H]⁺.

¹H-NMR (400 MHz, DMSO-d₆), δ [ppm]=13.17 (br. s., 1H), 11.29-10.84 (m, 1H), 9.57 (s, 1H), 8.86-8.16 (m, 1H), 7.87-7.73 (m, 1H), 7.49-7.30 (m, 2H), 3.73 (br. s., 1H), 2.81 (d, 3H), 2.42 (br. s., 1H), 1.94 (d, 4H), 1.61-1.20 (m, 4H).

### Example 7

### N⁵-{trans-4-[(2-bromo-4-fluorophenyl)carbamoyl]cyclohexyl}-N⁴-methyl-1H-imidazole-4,5-dicarboxamide

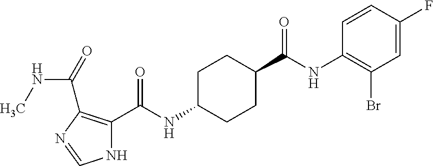 [SMILES: CNC(=O)c1nc[nH]c1C(=O)N[C@H]1CC[C@H](C(=O)Nc2ccc(F)cc2Br)CC1]

### Step 1:

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 31 μl (0.28 mmol) 2-bromo-4-chloroaniline were added and the reaction was stirred at room temperature for 30 minutes.

### Step 2

To the crude reaction mixture were added 5 ml tetrahydrofuran and 700 μl (2 M in tetrahydrofuran, 1.40 mmol) methanamine and the mixture was stirred for 2.5 hours at 50° C. The reaction mixture was concentrated in vacuo and the residue was dissolved in tetrahydrofuran. Water was added and the resulting precipitate was filtered off and was washed with water. The crude product was triturated with water and DMSO to give after drying under vacuum at 55° C. 61 mg of the title compound as a solid material.

LC-MS (Method 2): Rₜ=0.97 min; MS (ESIpos) m/z=468.0 [M+H]⁺.

¹H-NMR (400 MHz, DMSO-d₆), δ [ppm]=13.14 (br. s., 1H), 11.42-10.82 (m, 1H), 9.43 (br. s., 1H), 8.73-8.24 (m, 1H), 7.78 (s, 1H), 7.69-7.57 (m, 1H), 7.50 (dd, 1H), 7.33-7.11 (m, 1H), 3.71 (br. s., 1H), 2.81 (br. s., 3H), 2.13-1.82 (m, 4H), 1.66-1.16 (m, 4H).

### Example 8

### N⁵-{trans-4-[(4-fluorophenyl)carbamoyl]cyclohexyl}-N⁴-methyl-1H-imidazole-4,5-dicarboxamide

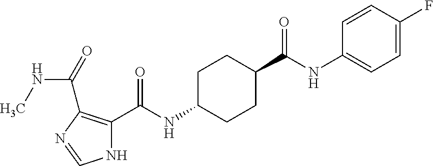 [SMILES: CNC(=O)c1nc[nH]c1C(=O)N[C@H]1CC[C@H](C(=O)Nc2ccc(F)cc2)CC1]

To a suspension of 100 mg (0.28 mmol) trans-4-({[4-(phenoxycarbonyl)-1H-imidazol-5-yl]carbonyl}amino)cyclohexane-carboxylic acid in 5 ml dichloromethane 62 μl (0.45 mmol) 1-chloro-N,N,2-trimethylprop-1-en-1-amine were added and the mixture was stirred at room temperature for 30 minutes. 68 μl (0.84 mmol) pyridine and 27 μl (0.28 mmol) 4-chloroaniline were added and the reaction was stirred at room temperature for 30 minutes.

### Step 2

To the crude reaction mixture were added 5 ml tetrahydrofuran and 700 μl (2 M in tetrahydrofuran, 1.40 mmol) methanamine and the mixture was stirred for 2.5 hours at 50° C. The reaction mixture was concentrated in vacuo and the residue was dissolved in tetrahydrofuran. Water was added and the resulting precipitate was filtered off and was washed with water. The crude product was triturated with water and DMSO to give after drying under vacuum at 55° C. 55 mg of the title compound as a solid material.

LC-MS (Method 2): Rₜ=0.89 min; MS (ESIpos) m/z=388.1 [M+H]⁺.

¹H-NMR (400 MHz, DMSO-d₆), δ [ppm]=13.13 (br. s., 1H), 11.33-10.76 (m, 1H), 9.88 (br. s., 1H), 8.66-8.30 (m, 1H), 7.78 (s, 1H), 7.61 (dd, 2H), 7.11 (t, 2H), 3.70 (br. s., 1H), 2.80 (br. s., 3H), 2.32 (br. s., 1H), 2.10-1.79 (m, 4H), 1.65-1.18 (m, 4H).

### Example 9

### N⁵-{trans-4-[(2-chloro-4-methylphenyl)carbamoyl]cyclohexyl}-N⁴-methyl-1H-imidazole-4,5-dicarboxamide

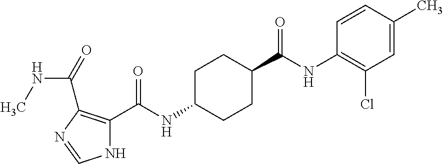

### Step 1:

To a suspension of 250 mg (0.70 mmol) trans-4-({[4-(phenoxycarbonyl)-1H-imidazol-5-yl]carbonyl}amino)cyclohexane-carboxylic acid in 20 ml dichloromethane 195 μl (1.40 mmol) 1-chloro-N,N,2-trimethylprop-1-en-1-amine were added and the mixture was stirred at room temperature for 1 hour. 170 μl (2.10 mmol) pyridine and 88 μl (0.70 mmol) 2-chloro-4-methylaniline were added and the reaction was stirred at room temperature for 24 hours. The reaction mixture was concentrated in vacuo to obtain the crude product which was used in the next step without further purification.

### Step 2

The crude product was suspended in 10 ml tetrahydrofuran and 2.18 ml (2 M in tetrahydrofuran, 4.36 mmol) methanamine were added. The suspension was stirred for 2.5 hours at room temperature. The reaction mixture was concentrated in vacuo and the residue was purified by flash column chromatography (dichloromethane/methanol-gradient) to give after trituration with dichloromethane 136 mg of the title compound as a solid material.

LC-MS (Method 1): Rₜ=1.07 min; MS (ESIpos) m/z=418.1 [M+H]⁺.

¹H-NMR (400 MHz, DMSO-d₆), δ [ppm]=13.15 (br. s., 1H), 11.28-10.94 (m, 1H), 9.33 (br. s., 1H), 8.67-8.29 (m, 1H), 7.79 (s, 1H), 7.47 (d, 1H), 7.30 (s, 1H), 7.15-7.08 (m, 1H), 5.74 (s, 1H), 3.71 (br. s., 1H), 2.81 (br. s., 3H), 2.44 (d, 1H), 2.30-2.22 (m, 3H), 2.10-1.77 (m, 4H), 1.66-1.13 (m, 4H).

### Example 10

### N⁵-{trans-4-[(2-chloropyridin-3-yl)carbamoyl]cyclohexyl}-N⁴-methyl-1H-imidazole-4,5-dicarboxamide

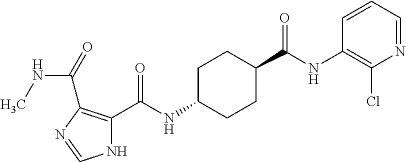 [SMILES: CNC(=O)c1nc[nH]c1C(=O)N[C@H]1CC[C@H](C(=O)Nc2cccnc2Cl)CC1]

### Step 1:

To a suspension of 250 mg (0.70 mmol) trans-4-({[4-(phenoxycarbonyl)-1H-imidazol-5-yl]carbonyl}amino)cyclohexane-carboxylic acid in 20 ml dichloromethane 195 μl (1.40 mmol) 1-chloro-N,N,2-trimethylprop-1-en-1-amine were added and the mixture was stirred at room temperature for 1 hour. 170 μl (2.10 mmol) pyridine and 91.7 mg (0.70 mmol) 2-chloropyridin-3-amine were added and the reaction was stirred at room temperature for 24 hours. The reaction mixture was concentrated in vacuo to obtain the crude product which was used in the next step without further purification.

### Step 2

The crude product was suspended in 10 ml tetrahydrofuran and 2.12 ml (2 M in tetrahydrofuran, 4.23 mmol) methanamine were added. The suspension was stirred for 24 hours at room temperature. The reaction mixture was concentrated in vacuo and the residue was purified by flash column chromatography (dichloromethane/methanol-gradient) to give after trituration with dichloromethane 125 mg of the title compound as a solid material.

LC-MS (Method 1): Rₜ=0.84 min; MS (ESIpos) m/z=403.3 [M+H]⁺.

¹H-NMR (400 MHz, DMSO-d₆), δ [ppm]=13.16 (br. s., 1H), 11.36-10.75 (m, 1H), 9.69-9.53 (m, 1H), 8.72-8.31 (m, 1H), 8.19 (dd, 1H), 8.12 (dd, 1H), 7.82-7.74 (m, 1H), 7.42 (dd, 1H), 5.74 (s, 1H), 3.73 (br. s., 1H), 2.81 (d, 3H), 2.09-1.82 (m, 4H), 1.67-1.21 (m, 4H).

### Example 11

### N⁵-{trans-4-[(3-chloropyridin-4-yl)carbamoyl]cyclohexyl}-N⁴-methyl-1H-imidazole-4,5-dicarboxamide

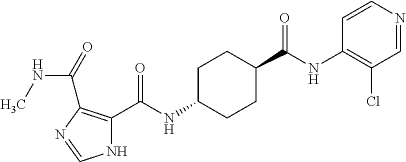 [SMILES: CNC(=O)c1nc[nH]c1C(=O)N[C@H]1CC[C@H](C(=O)Nc2ccncc2Cl)CC1]

390 mg (0.83 mmol) phenyl 5-({trans-4-[(3-chloropyridin-4-yl)carbamoyl]cyclohexyl}carbamoyl)-1H-imidazole-4-carboxylate were suspended in 10 ml tetrahydrofuran and 2.50 ml (2 M in tetrahydrofuran, 5.00 mmol) methanamine were added. The suspension was stirred for 24 hours at room temperature. The reaction mixture was concentrated in vacuo and the residue was purified by flash column chromatography (dichloromethane/methanol-gradient) to give after trituration with dichloromethane 41 mg (95% purity) of the title compound as a solid material.

LC-MS (Method 5): Rₜ=0.81 min; MS (ESIpos) m/z=406 [M+H]⁺.

¹H-NMR (400 MHz, DMSO-d₆), δ [ppm]=13.00-12.78 (m, 1H), 11.21-10.90 (m, 1H), 9.41-8.96 (m, 1H), 8.57 (s, 1H), 8.41 (d, 1H), 8.04 (d, 1H), 7.72 (s, 1H), 3.86-3.71 (m, 1H), 2.86 (d, 3H), 2.71-2.64 (m, 1H), 2.14-1.93 (m, 4H), 1.68-1.35 (m, 4H).

### Example 12

### N⁵-{trans-4-[(2-chloro-4-fluorophenyl)carbamoyl]cyclohexyl}-N⁴-[2-(4-methylpiperidin-1-yl)ethyl]-1H-imidazole-4,5-dicarboxamide

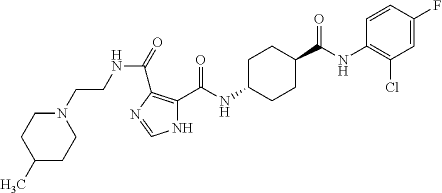 [SMILES: CC1CCN(CCNC(=O)c2nc[nH]c2C(=O)N[C@H]2CC[C@H](C(=O)Nc3ccc(F)cc3Cl)CC2)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 393 μl (2.47 mmol) 2-(4-methylpiperidin-1-yl)ethanamine. The suspension was stirred for 24 hours at room temperature. The reaction mixture was concentrated in vacuo and the residue was purified by flash column chromatography (dichloromethane/methanol-gradient) to give after trituration with diethyl ether 133 mg (96% purity) of the title compound as a solid material.

LC-MS (Method 5): Rₜ=0.89 min; MS (ESIpos) m/z=533 [M+H]⁺.

¹H-NMR (400 MHz, DMSO-d₆), δ [ppm]=13.13 (br. s., 1H), 11.13 (br. s., 1H), 9.46 (s, 1H), 8.39 (br. s., 1H), 7.86-7.71 (m, 1H), 7.59 (dd, 1H), 7.48 (dd, 1H), 7.20 (td, 1H), 3.72 (br. s., 1H), 3.38 (q, 2H), 2.84 (d, 2H), 2.43 (t, 3H), 2.10-1.84 (m, 6H), 1.55 (d, 5H), 1.31 (td, 2H), 1.20-1.02 (m, 2H), 0.87 (d, 3H).

### Example 13

### N⁵-{trans-4-[(2-chloro-4-fluorophenyl)carbamoyl]cyclohexyl}-N⁴-[2-(morpholin-4-yl)ethyl]-1H-imidazole-4,5-dicarboxamide

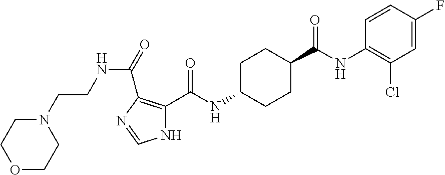 [SMILES: O=C(NCCN1CCOCC1)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 325 μl (2.47 mmol) 2-(morpholin-4-yl)ethanamine. The suspension was stirred for 24 hours at room temperature. The reaction mixture was concentrated in vacuo and the residue was purified by flash column chromatography (dichloromethane/methanol-gradient) to give after trituration with diethyl ether 161 mg (96% purity) of the title compound as a solid material.

LC-MS (Method 5): Rₜ=0.81 min; MS (ESIpos) m/z=521 [M+H]⁺.

¹H-NMR (400 MHz, DMSO-d₆), δ [ppm]=13.11 (br. s., 1H), 11.13 (br. s., 1H), 9.48-9.37 (m, 2H), 8.46 (br. s., 1H), 7.83-7.74 (m, 1H), 7.66-7.55 (m, 1H), 7.48 (dd, 1H), 7.20 (td, 1H), 3.85 (s, 1H), 3.80-3.63 (m, 1H), 3.62-3.52 (m, 4H), 3.41 (q, 2H), 2.47-2.35 (m, 5H), 2.09-1.85 (m, 4H), 1.81-1.70 (m, 1H), 1.64-1.22 (m, 4H).

### Example 14

### N-{trans-4-[(2-chloro-4-fluorophenyl)carbamoyl]cyclohexyl}-4-(2-oxa-6-azaspiro[3.3]hept-6-ylcarbonyl)-1H-imidazole-5-carboxamide

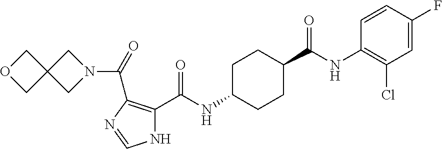 [SMILES: O=C(N[C@H]1CC[C@H](C(=O)Nc2ccc(F)cc2Cl)CC1)c1[nH]cnc1C(=O)N1CC2(COC2)C1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 468 mg (2.47 mmol) 2-oxa-6-azaspiro[3.3]heptane ethanedioate. The suspension was stirred for 8 hours at room temperature and for 24 hours at 60° C. The reaction mixture was concentrated in vacuo and the residue was purified by flash column chromatography (dichloromethane/methanol-gradient) to give after trituration with dichloromethane 107 mg of the title compound as a solid material.

LC-MS (Method 1): Rₜ=0.97 min; MS (ESIpos) m/z=490.1 [M+H]⁺.

¹H-NMR (400 MHz, DMSO-d₆), δ [ppm]=13.23 (br. s., 1H), 11.13 (d, 1H), 9.48 (s, 1H), 7.81 (s, 1H), 7.68-7.44 (m, 2H), 7.22 (td, 1H), 4.77 (s, 2H), 4.75-4.60 (m, 4H), 4.25 (s, 2H), 3.79-3.63 (m, 1H), 2.48-2.40 (m, 1H), 2.09-1.85 (m, 4H), 1.73-1.52 (m, 2H), 1.40-1.10 (m, 2H).

### Example 15

### N⁵-{trans-4-[(2-chloro-4-fluorophenyl)carbamoyl]cyclohexyl}-N⁴-ethyl-1H-imidazole-4,5-dicarboxamide

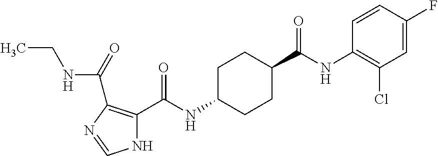 [SMILES: CCNC(=O)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 1.24 ml (2 M in tetrahydrofuran, 2.47 mmol) of an ethylamine solution. The suspension was stirred for 24 hours at room temperature. The reaction mixture was concentrated in vacuo and the residue was purified by flash column chromatography (dichloromethane/methanol-gradient) to give after trituration with diethyl ether 69 mg of the title compound as a solid material.

LC-MS (Method 5): Rₜ=1.11 min; MS (ESIpos) m/z=436 [M+H]⁺.

¹H-NMR (400 MHz, DMSO-d₆), δ [ppm]=13.16 (br. s., 1H), 11.26-10.94 (m, 1H), 9.46 (br. s., 1H), 8.71-8.17 (m, 1H), 7.78 (s, 1H), 7.59 (dd, 1H), 7.48 (dd, 1H), 7.20 (td, 1H), 3.89-3.57 (m, 1H), 3.39-3.30 (m, 2H), 2.46-2.41 (m, 1H), 2.13-1.84 (m, 4H), 1.63-1.17 (m, 5H), 1.17-1.04 (m, 3H).

### Example 16

### N⁵-{trans-4-[(2-chloro-4-fluorophenyl)carbamoyl]cyclohexyl}-N⁴,N⁴-dimethyl-1H-imidazole-4,5-dicarboxamide

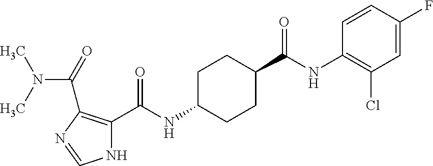 [SMILES: CN(C)C(=O)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 1.24 ml (2 M in tetrahydrofuran, 2.47 mmol) of an N-methylmethanamine solution. The suspension was stirred for 24 hours at room temperature. The reaction mixture was concentrated in vacuo and the residue was purified by flash column chromatography (dichloromethane/methanol-gradient) to give after trituration with dichloromethane 119 mg of the title compound as a solid material.

LC-MS (Method 2): Rₜ=0.88 min; MS (ESIpos) m/z=436.1 [M+H]⁺.

¹H-NMR (400 MHz, DMSO-d₆), δ [ppm]=12.81 (br. s., 1H), 9.97-9.80 (m, 1H), 9.15 (s, 1H), 7.70 (s, 1H), 7.66 (dd, 1H), 7.45-7.34 (m, 1H), 7.18 (td, 1H), 3.72 (m, 1H), 3.05 (s, 6H), 2.48-2.42 (m, 1H), 2.10-1.89 (m, 4H), 1.67-1.50 (m, 2H), 1.43-1.25 (m, 2H).

### Example 17

### N⁵-{trans-4-[(2-chloro-4-fluorophenyl)carbamoyl]cyclohexyl}-N⁴-cyclopropyl-1H-imidazole-4,5-dicarboxamide

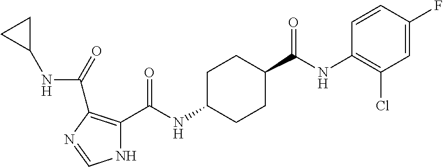 [SMILES: O=C(NC1CC1)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 171 μl (2.47 mmol) cyclopropanamine. The suspension was stirred for 24 hours at room temperature. The reaction mixture was concentrated in vacuo and the residue was purified by flash column chromatography (dichloromethane/methanol-gradient) to give after trituration with dichloromethane 129 mg (95% purity) of the title compound as a solid material.

LC-MS (Method 5): Rₜ=1.11 min; MS (ESIpos) m/z=448 [M+H]⁺.

¹H-NMR (400 MHz, DMSO-d₆), δ [ppm]=13.18 (br. s., 1H), 11.37-10.83 (m, 1H), 9.47 (s, 1H), 8.63-8.30 (m, 1H), 7.78 (br. s., 1H), 7.60 (dd, 1H), 7.48 (dd, 1H), 7.20 (td, 1H), 5.74 (s, 1H), 3.74 (br. s., 1H), 2.86 (br. s., 1H), 2.46-2.40 (m, 1H), 2.08-1.83 (m, 4H), 1.62-1.30 (m, 4H), 0.82-0.42 (m, 4H).

### Example 18

### N⁵-{trans-4-[(2-chloro-4-fluorophenyl)carbamoyl]cyclohexyl}-N⁴-(2-hydroxyethyl)-1H-imidazole-4,5-dicarboxamide

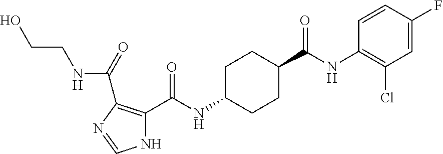 [SMILES: O=C(NCCO)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 149 μl (2.47 mmol) 2-aminoethanol. The suspension was stirred for 24 hours at room temperature. The reaction mixture was concentrated in vacuo and the residue was purified by flash column chromatography (dichloromethane/methanol-gradient) to give after trituration with dichloromethane 137 mg of the title compound as a solid material.

LC-MS (Method 5): Rₜ=0.95 min; MS (ESIpos) m/z=452 [M+H]⁺.

¹H-NMR (400 MHz, DMSO-d₆), δ [ppm]=12.77-12.15 (m, 1H), 11.17 (br. s., 1H), 9.51 (s, 1H), 8.55-8.32 (m, 1H), 7.82 (s, 1H), 7.61 (dd, 1H), 7.50 (dd, 1H), 7.22 (td, 1H), 4.82 (br. s., 1H), 3.74 (br. s., 1H), 3.56-3.47 (m, 2H), 3.41-3.35 (m, 2H), 2.49-2.40 (m, 1H), 2.09-1.83 (m, 4H), 1.65-1.21 (m, 4H).

### Example 19

### N⁵-{trans-4-[(2-chloro-4-fluorophenyl)carbamoyl]cyclohexyl}-N⁴-(2-methoxyethyl)-1H-imidazole-4,5-dicarboxamide

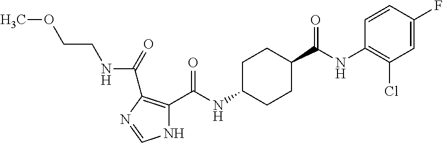 [SMILES: COCCNC(=O)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 215 μl (2.47 mmol) 2-methoxyethanamine. The suspension was stirred for 24 hours at room temperature. The reaction mixture was concentrated in vacuo and the residue was purified by flash column chromatography (dichloromethane/methanol-gradient) to give after trituration with dichloromethane 24 mg of the title compound as a solid material.

LC-MS (Method 6): Rₜ=1.07 min; MS (ESIpos) m/z=466 [M+H]⁺.

¹H-NMR (400 MHz, DMSO-d₆), δ [ppm] 13.20 (br. s., 1H), 11.25-10.99 (m, 1H), 9.47 (d, 1H), 8.56-8.28 (m, 1H), 7.80 (s, 1H), 7.59 (dd, 1H), 7.52-7.41 (m, 1H), 7.20 (td, 1H), 3.86-3.66 (m, 1H), 3.46 (d, 4H), 3.27 (s, 3H), 2.43 (br. s., 1H), 2.09-1.82 (m, 4H), 1.67-1.44 (m, 3H), 1.38-1.09 (m, 2H).

### Example 20

### N⁵-{trans-4-[(2-chloro-4-fluorophenyl)carbamoyl]cyclohexyl}-N⁴-isopropyl-1H-imidazole-4,5-dicarboxamide

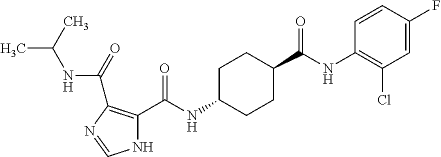 [SMILES: CC(C)NC(=O)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 215 μl (2.47 mmol) propan-2-amine. The suspension was stirred for 6 hours at room temperature. The reaction mixture was concentrated in vacuo and the residue was purified by flash column chromatography (dichloromethane/methanol-gradient) to give after trituration with diethyl ether 175 mg of the title compound as a solid material.

LC-MS (Method 5): Rₜ=1.17 min; MS (ESIpos) m/z=450 [M+H]⁺.

¹H-NMR (400 MHz, DMSO-d₆), δ [ppm]=13.21 (br. s., 1H), 11.30-10.91 (m, 1H), 9.51 (d, 1H), 8.51-8.19 (m, 1H), 7.81 (s, 1H), 7.69-7.56 (m, 1H), 7.56-7.44 (m, 1H), 7.22 (td, 1H), 4.25-3.94 (m, 1H), 3.87-3.62 (m, 1H), 2.45 (br. s., 1H), 2.20-1.79 (m, 4H), 1.64-1.23 (m, 4H), 1.20 (s, 3H), 1.18 (s, 3H).

### Example 21

### N⁵-{trans-4-[(2-chloro-4-fluorophenyl)carbamoyl]cyclohexyl}-N⁴-(2,2,2-trifluoroethyl)-1H-imidazole-4,5-dicarboxamide

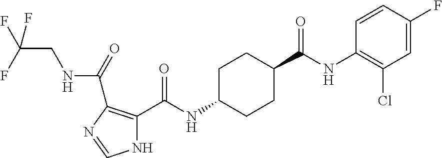 [SMILES: O=C(NCC(F)(F)F)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 194 μl (2.47 mmol) 2,2,2-trifluoroethanamine. The suspension was stirred for 4 days at 130° C. in a pressure tube. The reaction mixture was concentrated in vacuo and the residue was purified by flash column chromatography (dichloromethane/methanol-gradient) to give after trituration with dichloromethane 111 mg of the title compound as a solid material.

LC-MS (Method 5): Rₜ=1.20 min; MS (ESIpos) m/z=490 [M+H]⁺.

¹H-NMR (400 MHz, DMSO-d₆), δ [ppm]=13.43 (br. s., 1H), 12.02-10.57 (m, 1H), 9.52 (br. s., 1H), 9.26-8.48 (m, 1H), 7.91 (br. s., 1H), 7.61 (dd, 1H), 7.50 (dd, 1H), 7.22 (td, 1H), 4.30-4.00 (m, 2H), 3.92-3.63 (m, 1H), 2.47-2.41 (m, 1H), 2.14-1.82 (m, 4H), 1.32 (br. s., 4H).

### Example 22

### N-{trans-4-[(2-chloro-4-fluorophenyl)carbamoyl]cyclohexyl}-4-[(1,1-dioxido-1-thia-6-azaspiro[3.3]hept-6-yl)carbonyl]-1H-imidazole-5-carboxamide

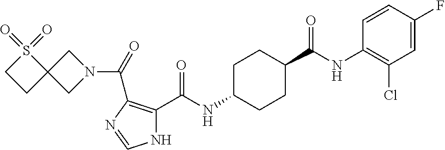 [SMILES: O=C(N[C@H]1CC[C@H](C(=O)Nc2ccc(F)cc2Cl)CC1)c1[nH]cnc1C(=O)N1CC2(CCS2(=O)=O)C1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 454 mg (2.47 mmol) 1-thia-6-azaspiro[3.3]heptane 1,1-dioxide hydrochloride and 345 μl triethylamine. The suspension was stirred for 6 hours at room temperature. The reaction mixture was concentrated in vacuo and the residue was purified by flash column chromatography (dichloromethane/methanol-gradient) to give after trituration with a mixture of dichloromethane/dioxane/methanol (1:1:1) 145 mg of the title compound as a solid material.

LC-MS (Method 5): Rₜ=1.03 min; MS (ESIpos) m/z=528 [M+H]⁺.

¹H-NMR (400 MHz, DMSO-d₆), δ [ppm]=13.26 (br. s., 1H), 10.92 (d, 1H), 9.49 (s, 1H), 7.84 (s, 1H), 7.60 (dd, 1H), 7.50 (dd, 1H), 7.22 (td, 1H), 5.11-4.81 (m, 2H), 4.53-4.32 (m, 2H), 4.13 (t, 2H), 3.71 (d, 1H), 2.48-2.36 (m, 3H), 2.14-1.88 (m, 4H), 1.57 (q, 2H), 1.42-1.17 (m, 2H).

### Example 23

### 4-(azetidin-1-ylcarbonyl)-N-{trans-4-[(2-chloro-4-fluorophenyl)carbamoyl]cyclohexyl}-1H-imidazole-5-carboxamide

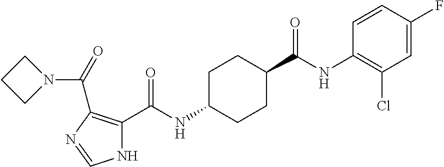 [SMILES: O=C(N[C@H]1CC[C@H](C(=O)Nc2ccc(F)cc2Cl)CC1)c1[nH]cnc1C(=O)N1CCC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 170 μl (2.47 mmol) azetidine. The suspension was stirred for 6 hours at room temperature. The reaction mixture was concentrated in vacuo and the residue was purified by flash column chromatography (dichloromethane/methanol-gradient) to give after trituration with dichloromethane 64 mg of the title compound as a solid material.

LC-MS (Method 5): Rₜ=1.07 min; MS (ESIpos) m/z=448 [M+H]⁺.

¹H-NMR (400 MHz, DMSO-d₆), δ [ppm]=13.16 (br. s., 1H), 11.23 (d, 1H), 9.49 (s, 1H), 7.79 (s, 1H), 7.60 (dd, 1H), 7.50 (dd, 1H), 7.22 (td, 1H), 4.62 (t, 2H), 4.08 (t, 2H), 3.77-3.60 (m, 1H), 2.47-2.39 (m, 1H), 2.36-2.19 (m, 2H), 2.09-1.86 (m, 4H), 1.64-1.46 (m, 2H), 1.38-1.19 (m, 2H).

### Example 24

### 4 N-{trans-4-[(2-chloro-4-fluorophenyl)carbamoyl]cyclohexyl}-4-[(3-methoxyazetidin-1-yl)carbonyl]-1H-imidazole-5-carboxamide

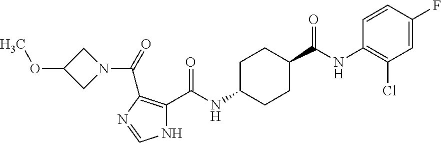 [SMILES: COC1CN(C(=O)c2nc[nH]c2C(=O)N[C@H]2CC[C@H](C(=O)Nc3ccc(F)cc3Cl)CC2)C1]

To a suspension of 150 mg (0.31 mmol) phenyl 5-({trans-4-[(2-chloro-4-fluorophenyl)carbamoyl]cyclohexyl}carbamoyl)-1H-imidazole-4-carboxylate in 5 ml tetrahydrofuran were added 115 mg (0.93 mmol) 3-methoxyazetidine hydrochloride and 129 μl (0.93 mmol) triethylamine. The suspension was stirred for 5 hours 60° C. The precipitate was filtered off and was triturated with dichloromethane. The residue was triturated diethyl ether to give 80 mg of the title compound as a solid material.

LC-MS (Method 1): Rₜ=1.04 min; MS (ESIpos) m/z=478.0 [M+H]⁺.

¹H-NMR (400 MHz, DMSO-d₆), δ [ppm]=13.15 (br. s., 1H), 11.12 (d, 1H), 9.49 (s, 1H), 7.81 (s, 1H), 7.60 (dd, 1H), 7.50 (dd, 1H), 7.22 (td, 1H), 4.79 (dd, 1H), 4.49-4.35 (m, 1H), 4.32-4.22 (m, 2H), 3.96-3.80 (m, 1H), 3.78-3.58 (m, 1H), 3.25 (s, 3H), 2.50-2.44 (m, 1H), 2.11-1.84 (m, 4H), 1.65-1.45 (m, 2H), 1.40-1.11 (m, 3H).

### Example 25

### N⁵-{trans-4-[(2-chloro-4-fluorophenyl)carbamoyl]cyclohexyl}-N⁴-[2-(1-oxa-6-azaspiro[3.3]hept-6-yl)ethyl]-1H-imidazole-4,5-dicarboxamide

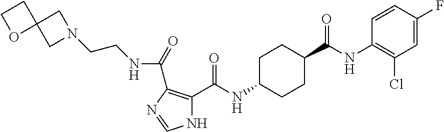 [SMILES: O=C(NCCN1CC2(CCO2)C1)c1nc[nH]c1C(=O)N[C@H]1CC[C@H](C(=O)Nc2ccc(F)cc2Cl)CC1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 634 mg (2.48 mmol) 2-(1-oxa-6-azaspiro[3.3]hept-6-yl)ethanamine trifluoroacetate and 517 μl (3.71 mmol) triethylamine. The suspension was stirred for 3 hours 55° C. The precipitate was filtered off. The residue was triturated with dichloromethane to give after drying under vacuum 101 mg of the title compound as a solid material.

LC-MS (Method 1): Rₜ=0.83 min; MS (ESIpos) m/z=533.1 [M+H]⁺.

¹H-NMR (400 MHz, DMSO-d₆), δ [ppm]=11.19-11.00 (m, 1H), 9.46 (br. s., 1H), 8.39 (br. s., 1H), 7.79 (s, 1H), 7.60 (dd, 1H), 7.48 (dd, 1H), 7.20 (td, 1H), 4.35 (t, 2H), 3.71 (br. s., 1H), 3.63-3.48 (m, 2H), 3.23 (br. s., 2H), 3.04 (d, 2H), 2.72 (t, 2H), 2.09-1.83 (m, 4H), 1.79-1.70 (m, 1H), 1.64-1.17 (m, 5H).

### Example 26

### N-{trans-4-[(2-chloro-4-fluorophenyl)carbamoyl]cyclohexyl}-4-{[3-(dimethylamino)azetidin-1-yl]carbonyl}-1H-imidazole-5-carboxamide

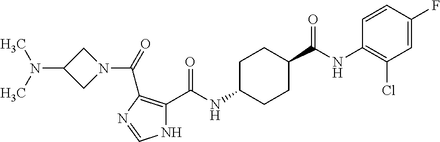 [SMILES: CN(C)C1CN(C(=O)c2nc[nH]c2C(=O)N[C@H]2CC[C@H](C(=O)Nc3ccc(F)cc3Cl)CC2)C1]

To a suspension of 200 mg (0.41 mmol) phenyl 5-({trans-4-[(2-chloro-4-fluorophenyl)carbamoyl]cyclohexyl}carbamoyl)-1H-imidazole-4-carboxylate in 6 ml tetrahydrofuran were added 428 mg (2.48 mmol) N,N-dimethylazetidin-3-amine dihydrochloride and 690 μl (4.95 mmol) triethylamine. The suspension was stirred for 2 hours 55° C. The precipitate was filtered off and was washed with dichloromethane. The residue was triturated with water to give 124 mg of the title compound as a solid material.

LC-MS (Method 1): Rₜ=0.79 min; MS (ESIpos) m/z=491.1 [M+H]⁺.

¹H-NMR (400 MHz, DMSO-d₆), δ [ppm]=13.19 (br. s., 1H), 11.14 (d, 1H), 9.45 (s, 1H), 7.78 (s, 1H), 7.59 (dd, 1H), 7.48 (dd, 1H), 7.20 (td, 1H), 4.62 (dd, 1H), 4.34 (dd, 1H), 4.08 (dd, 1H), 3.83 (dd, 1H), 3.75-3.50 (m, 1H), 3.12-2.97 (m, 1H), 2.47-2.40 (m, 1H), 2.10 (s, 6H), 2.06-1.86 (m, 4H), 1.65-1.45 (m, 2H), 1.37-1.17 (m, 2H).

### Example 27

### N⁵-{trans-4-[(6-bromo-2,3,4-trifluorophenyl)carbamoyl]cyclohexyl}-N⁴-methyl-1H-imidazole-4,5-dicarboxamide

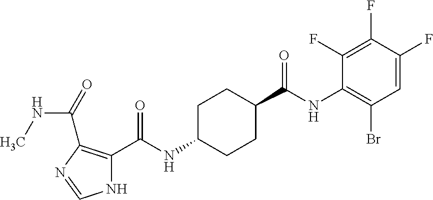 [SMILES: CNC(=O)c1nc[nH]c1C(=O)N[C@H]1CC[C@H](C(=O)Nc2c(Br)cc(F)c(F)c2F)CC1]

### Step 1:

To a suspension of 200 mg (0.56 mmol) trans-4-({[4-(phenoxycarbonyl)-1H-imidazol-5-yl]carbonyl}amino)cyclohexane-carboxylic acid in 10 ml dichloromethane 156 μl (1.12 mmol) 1-chloro-N,N,2-trimethylprop-1-en-1-amine were added and the mixture was stirred at room temperature for 75 minutes. 136 μl (1.68 mmol) pyridine and 126 mg (0.56 mmol) 6-bromo-2,3,4-trifluoroaniline were added and the reaction was stirred at room temperature for 30 minutes.

### Step 2

To the crude reaction mixture were added 1.4 ml (2 M in tetrahydrofuran, 2.80 mmol) of a methanamine solution in tetrahydrofuran and the mixture was stirred for 3 days at room temperature. The reaction mixture was concentrated in vacuo and the residue was purified by flash column chromatography to give after washing with methanol 23 mg of the title compound as a solid material.

LC-MS (Method 6): Rₜ=0.93 min; MS (ESIpos) m/z=504.1 [M+H]⁺.

¹H-NMR (400 MHz, DMSO-d₆), δ [ppm]=13.21 (br. s., 1H), 11.38-10.95 (m, 1H), 9.82 (s, 1H), 8.74-8.32 (m, 1H), 7.90 (ddd, 1H), 7.84-7.78 (m, 1H), 5.77 (s, 0.1H), 3.74 (br. s., 1H), 2.82 (d, 3H), 2.46 (br. s., 1H), 1.98 (d, 4H), 1.64-1.15 (m, 5H).

### Example 28

### N⁵-{trans-4-[(6-bromo-2,4-difluorophenyl)carbamoyl]cyclohexyl}-N⁴-methyl-1H-imidazole-4,5-dicarboxamide

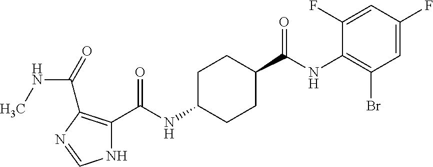 [SMILES: CNC(=O)c1nc[nH]c1C(=O)N[C@H]1CC[C@H](C(=O)Nc2c(F)cc(F)cc2Br)CC1]

### Step 1:

To a suspension of 200 mg (0.56 mmol) trans-4-({[4-(phenoxycarbonyl)-1H-imidazol-5-yl]carbonyl}amino)cyclohexane-carboxylic acid in 10 ml dichloromethane 156 μl (1.12 mmol) 1-chloro-N,N,2-trimethylprop-1-en-1-amine were added and the mixture was stirred at room temperature for 75 minutes. 136 μl (1.68 mmol) pyridine and 116 mg (0.56 mmol) 6-bromo-2,4-difluoroaniline were added and the reaction was stirred at room temperature for 30 minutes.

### Step 2

To the crude reaction mixture were added 1.4 ml (2 M in tetrahydrofuran, 2.80 mmol) of a methanamine solution in tetrahydrofuran and the mixture was stirred for 3 days at room temperature. The reaction mixture was concentrated in vacuo and the residue was purified by flash column chromatography to give after washing with methanol 85 mg of the title compound as a solid material.

LC-MS (Method 6): Rₜ=0.88 min; MS (ESIpos) m/z=484.1 [M+H]⁺.

¹H-NMR (400 MHz, DMSO-d₆), δ [ppm]=13.22 (br. s., 1H), 11.36-10.83 (m, 1H), 9.60 (s, 1H), 8.91-8.08 (m, 1H), 7.81 (s, 1H), 7.63-7.54 (m, 1H), 7.45 (td, 1H), 3.74 (br. s., 1H), 2.82 (d, 3H), 2.43 (br. s., 1H), 2.12-1.80 (m, 4H), 1.69-1.23 (m, 4H).

### Example 29

### N⁵-{trans-4-[(2-chloro-4-fluorophenyl)carbamoyl]cyclohexyl}-N⁴-methyl-1,3-oxazole-4,5-dicarboxamide

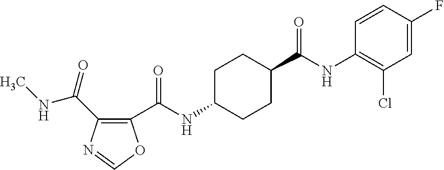 [SMILES: CNC(=O)c1ncoc1C(=O)N[C@H]1CC[C@H](C(=O)Nc2ccc(F)cc2Cl)CC1]

To a suspension of 186 mg (0.44 mmol) methyl 5-({trans-4-[(2-chloro-4-fluorophenyl)carbamoyl]cyclohexyl}carbamoyl)-1,3-oxazole-4-carboxylatein 2.5 ml dioxane were added 1.3 ml (2M in tetrahydrofuran, 2.63 mmol) of a methanamine solution in tetrahydrofuran and the mixture was stirred for 24 hours at 80° C. The reaction mixture was concentrated in vacuo and the residue was purified by flash column chromatography (three times) to give 10 mg of the title compound as a solid material.

LC-MS (Method 1): Rₜ=1.03 min; MS (ESIpos) m/z=443.8 [M+H]⁺.

¹H-NMR (400 MHz, DMSO-d₆), b [ppm]=10.93 (d, 1H), 9.51 (s, 1H), 9.03 (d, 1H), 8.74-8.64 (m, 1H), 7.60 (dd, 1H), 7.51 (dd, 1H), 7.22 (td, 1H), 3.80-3.57 (m, 1H), 2.86-2.80 (m, 3H), 2.49-2.43 (m, 1H), 2.10-1.89 (m, 4H), 1.64-1.19 (m, 5H).

### Example 30

### methyl 4-({trans-4-[(2-chloro-4-fluorophenyl)carbamoyl]cyclohexyl}carbamoyl)-1,3-oxazole-5-carboxylate

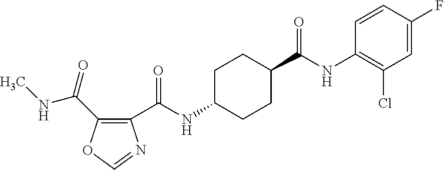 [SMILES: CNC(=O)c1ocnc1C(=O)N[C@H]1CC[C@H](C(=O)Nc2ccc(F)cc2Cl)CC1]

To a suspension of 210 mg (0.50 mmol) methyl 4-({trans-4-[(2-chloro-4-fluorophenyl)carbamoyl]cyclohexyl}carbamoyl)-1,3-oxazole-5-carboxylate in 3 ml dioxane were added 1.5 ml (2M in tetrahydrofuran, 3.00 mmol) of a methanamine solution in tetrahydrofuran and the mixture was stirred for 24 hours at 80° C. The reaction mixture was concentrated in vacuo and the residue was purified by flash column chromatography (twice) to give after recrystallization (dichloromethane) 16 mg of the title compound as a solid material.

LC-MS (Method 2): Rₜ=1.03 min; MS (ESIpos) m/z=423.1 [M+H]⁺.

¹H-NMR (400 MHz, DMSO-d₆), b [ppm]=10.61 (d, 1H), 9.51 (s, 1H), 9.00 (d, 1H), 8.71 (s, 1H), 7.62 (dd, 1H), 7.50 (dd, 1H), 7.22 (td, 1H), 5.76 (s, 0.4H), 3.90-3.72 (m, 1H), 2.86 (d, 3H), 2.44 (d, 1H), 2.00-1.83 (m, 4H), 1.63-1.40 (m, 4H).

### Example 31

### N⁴-{(1α,2α,4β)-4-[(2-chloro-4-fluorophenyl)carbamoyl]-2-fluorocyclohexyl}-N⁵-methyl-1H-imidazole-4,5-dicarboxamide

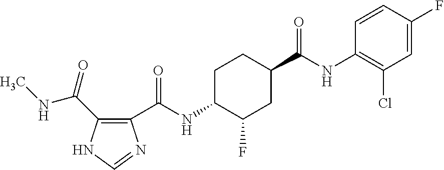 [SMILES: CNC(=O)c1[nH]cnc1C(=O)N[C@@H]1CC[C@@H](C(=O)Nc2ccc(F)cc2Cl)C[C@@H]1F]

903 mg (3.52 mmol) (mu-1,4-diazabicyclo[2.2.2]octane-kappaN1:kappaN4) (hexamethyl)dialuminium and 518 mg (3.52 mmol) 2-chloro-4-fluoroaniline in 12 ml dry tetrahydrofuran were stirred for 1 hour at 40° C. in a microwave vial. A solution of 230 mg (0.71 mmol) methyl (1α,3β,4β)-3-fluoro-4-({[4-(methylcarbamoyl)-1H-imidazol-5-yl]carbonyl}amino)cyclohexanecarboxylate in 2 ml dry tetrahydrofuran were added via a syringe and the mixture was stirred for 3 days at room temperature. The reaction mixture was poured into a mixture of icewater and ammoniumchloride solution. The aqueous phase was extracted three times with a mixture of dichloromethane/methanol (8:2). The combined organic phases were washed with saturated sodiumbicarbonate solution until a neutral pH was reached. The organic phase was dried over sodium sulphate, and the solvent was removed under reduced pressure. The crude product was purified by flash column chromatography (twice) to give after recrystallization (methanol) 15 mg of the title compound as a solid material (racemic mixture).

LC-MS (Method 2): Rₜ=0.95 min; MS (ESIpos) m/z=440.0 [M+H]⁺.

¹H-NMR (00 MHz, DMSO-d₆), b [ppm]=13.23 (br. s., 1H), 11.46 (d, 1H), 9.67 (s, 1H), 8.61 (br. s., 1H), 7.88-7.77 (m, 1H), 7.65-7.56 (m, 1H), 7.54-7.44 (m, 1H), 7.23 (td, 1H), 4.97 (d, 1H), 4.15-3.83 (m, 1H), 2.81 (d, 4H), 2.20 (br. s., 1H), 2.05-1.54 (m, 5H).

### Example 32

### N⁴-{(trans)-4-[(2-chloro-4-fluorophenyl)carbamoyl]-2,2-difluorocyclohexyl}-N⁵-methyl-1H-imidazole-4,5-dicarboxamide

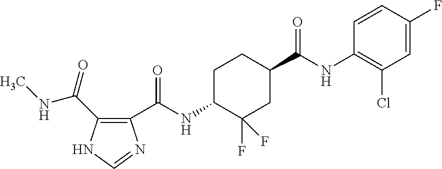 [SMILES: CNC(=O)c1[nH]cnc1C(=O)N[C@@H]1CC[C@@H](C(=O)Nc2ccc(F)cc2Cl)CC1(F)F]

744 mg (2.90 mmol) (mu-1,4-diazabicyclo[2.2.2]octane-kappaN1:kappaN4) (hexamethyl)dialuminium and 427 mg (2.90 mmol) 2-chloro-4-fluoroaniline in 10 ml dry tetrahydrofuran were stirred for 1 hour at 40° C. in a microwave vial. A solution of 200 mg (0.58 mmol) methyl (trans)-3,3-difluoro-4-({[4-(methylcarbamoyl)-1H-imidazol-5-yl]carbonyl}amino)cyclohexanecarboxylate in 2 ml dry tetrahydrofuran were added via a syringe and the mixture was stirred for 3 days at room temperature. The reaction mixture was poured into a mixture of icewater and ammoniumchloride solution. The aqueous phase was extracted three times with a mixture of dichloromethane/methanol (8:2). The combined organic phases were washed with saturated sodiumbicarbonate solution until a neutral pH was reached. The organic phase was dried over sodium sulphate, and the solvent was removed under reduced pressure. The crude product was purified by flash column chromatography (twice) to give after recrystallization (methanol) 116 mg of the title compound as racemic mixture.

Both enantiomers were separated by chiral HPLC to give 30 mg of enantiomer 1 (Example 33) and 17 mg enantiomer 2 (Example 34) of the title compound as solid materials.

Preparative chiral HPLC method: Instrument: Sepiatec: Prep SFC100; column: Chiralpak IC 5 μm 250×20 mm; eluent A: CO₂, eluent B: methanol; isocratic: 1 5% B; flow 80.0 ml/min temperature: 40° C.; BPR: 150 bar; MWD:254 nm

Chrial analytical HPLC method: Instrument: Agilent: 1260 AS, MWD, Aurora SFC-Modul; column: Chiralpak IC 5 μm 100×4.6 mm; eluent A: CO2, eluent B: methanol; isocratic: 15% B; flow 4.0 ml/min; temperature: 37.5° C.; injection: 5 μl; BPR: 100 bar; MWD:254 nm

### Example 33

### N⁴-{(trans)-4-[(2-chloro-4-fluorophenyl)carbamoyl]-2,2-difluorocyclohexyl}-N⁵-methyl-1H-imidazole-4,5-dicarboxamide (enantiomer 1)

Chiral HPLC (Method see Example 32): Rₜ=2.45 min, purity: 99.7% by UV.

LC-MS (Method 6): Rₜ=0.96 min; MS (ESIpos) m/z=458.1 [M+H]⁺.

¹H-NMR (400 MHz, DMSO-d₆), δ [ppm]=13.30 (br. s., 1H), 11.63 (d, 1H), 9.74 (s, 1H), 8.67 (d, 1H), 7.86 (s, 1H), 7.61 (dd, 1H), 7.53 (dd, 1H), 7.24 (td, 1H), 4.40 (d, 1H), 2.86-2.76 (m, 4H), 2.40-2.30 (m, 1H), 2.26-1.95 (m, 3H), 1.80-1.48 (m, 2H).

### Example 34

### N⁴-{(trans)-4-[(2-chloro-4-fluorophenyl)carbamoyl]-2,2-difluorocyclohexyl}-N⁵-methyl-1H-imidazole-4,5-dicarboxamide (enantiomer 2)

Chiral HPLC (Method see Example 32): Rₜ=3.96 min, purity: 97.7% by UV.

LC-MS (Method 6): Rₜ=0.96 min; MS (ESIpos) m/z=458.1 [M+H]⁺.

¹H-NMR (400 MHz, DMSO-d₆), δ [ppm]=13.30 (br. s., 1H), 11.63 (d, 1H), 9.74 (s, 1H), 8.67 (br. s., 1H), 7.86 (s, 1H), 7.61 (dd, 1H), 7.53 (dd, 1H), 7.24 (td, 1H), 4.51-4.24 (m, 1H), 2.86-2.81 (m, 4H), 2.40-2.29 (m, 1H), 2.26-1.95 (m, 3H), 1.81-1.49 (m, 2H).

### Example 35

### N⁴-{(1α,2β,4β)-4-[(2-chloro-4-fluorophenyl)carbamoyl]-2-methoxycyclohexyl}-N⁵-methyl-1H-imidazole-4,5-dicarboxamide

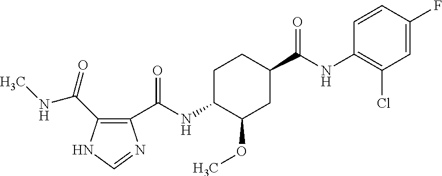 [SMILES: CNC(=O)c1[nH]cnc1C(=O)N[C@@H]1CC[C@@H](C(=O)Nc2ccc(F)cc2Cl)C[C@H]1OC]

795 mg (3.10 mmol) (mu-1,4-diazabicyclo[2.2.2]octane-kappaN1:kappaN4) (hexamethyl)dialuminium and 456 mg (3.10 mmol) 2-chloro-4-fluoroaniline in 10 ml dty tetrahydrofuran were stirred for 1 hour at 40° C. in a microwave vial. A solution of 210 mg (0.62 mmol) methyl (1α,3α,43)-3-methoxy-4-({[4-(methylcarbamoyl)-1H-imidazol-5-yl]carbonyl}amino)cyclohexanecarboxylate in 2 ml dry tetrahydrofuran were added via a syringe and the mixture was stirred for 24 hours at room temperature. The reaction mixture was poured into icewater and the aqueous phase was extracted three times with a mixture of dichloromethane/methanol (8:2). The combined organic phases were washed with saturated sodiumbicarbonate solution until a neutral pH was reached. The organic phase was dried over sodium sulphate, and the solvent was removed under reduced pressure. The crude product was purified by flash column chromatography (twice) to give after trituration with diethyl ether 54 mg of the title compound as racemate. The racemate was separated by preparative chrial HPLC to give 14 mg enantiomer 1 (Example 36) and 16 mg enantiomer 2 (Example 37).

Preparative chiral HPLC method: Instrument: Labomatic Pumpe HD-5000, Labomatic SP-3000, Labocord 5000, Labomatic Labcol Vario 4000, Gilson GX-241; column: Chiralpak IC 5 μm 250×30 mm; eluent A: ethanol, eluent B: methanol; eluent C: diethylamine, isocratic: 50:50:0.1 (v/v/v); flow 35.0 ml/min temperature: room temperature; MWD:254 nm

Chrial analytical HPLC method: Agilent 1260, column: Chiralpak IC 3 μm 100×4.6 mm, Solvent: methanol+0.1% diethylamine/ethanol 50:50 (v/v), flow: 1.0 ml/min, temperature: 25° C., injection: 5.0 μl, detection: DAD 254 nm.

### Example 36

### N⁴-{(1α,2β,4β)-4-[(2-chloro-4-fluorophenyl)carbamoyl]-2-methoxycyclohexyl}-N⁵-methyl-1H-imidazole-4,5-dicarboxamide (enantiomer 1)

Chiral HPLC (Method see Example 35): Rₜ=1.68 min, Purity: 99.9% by UV.

LC-MS (Method 6): Rₜ=0.89 min; MS (ESIpos) m/z=452.1 [M+H]⁺.

¹H-NMR (400 MHz, DMSO-d₆), δ [ppm]=13.15 (br. s., 1H), 11.28 (d, 1H), 9.51 (d, 1H), 8.66-8.40 (m, 1H), 7.79 (d, 1H), 7.61 (ddd, 1H), 7.48 (dt, 1H), 7.27-7.09 (m, 1H), 3.84-3.64 (m, 1H), 3.54-3.45 (m, 0.5H), 3.35-3.27 (m, 4H), 3.24-3.13 (m, 0.5H), 2.82 (dd, 3H), 2.62-2.52 (m, 1H), 2.40-2.19 (m, 1H), 2.13-1.81 (m, 2H), 1.65-1.19 (m, 3H).

### Example 37

### N⁴-{(1α,2β,4β)-4-[(2-chloro-4-fluorophenyl)carbamoyl]-2-methoxycyclohexyl}-N⁵-methyl-1H-imidazole-4,5-dicarboxamide (enantiomer 2)

Chiral HPLC (Method see Example 35): Rₜ=2.90 min, Purity: 99.6% by UV.

LC-MS (Method 6): Rₜ=0.89 min; MS (ESIpos) m/z=452.1 [M+H]⁺.

¹H-NMR (400 MHz, DMSO-d₆), δ [ppm]=13.20 (br. s., 1H), 11.57-10.89 (m, 1H), 9.56 (d, 1H), 8.92-8.37 (m, 1H), 7.82 (s, 1H), 7.62 (dd, 1H), 7.51 (dd, 1H), 7.23 (td, 1H), 3.72 (br. s., 1H), 3.51-3.14 (m, 4H), 2.83 (br. s., 3H), 2.37-2.28 (m, 1H), 2.18-1.76 (m, 2H), 1.66-1.23 (m, 4H).

### Example 38

### N⁴-{4-[(2-chloro-4-fluorophenyl)carbamoyl]-2-methylcyclohexyl}-N⁵-methyl-1H-imidazole-4,5-dicarboxamide (mixture of isomers)

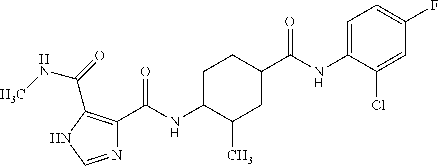 [SMILES: CNC(=O)c1[nH]cnc1C(=O)NC1CCC(C(=O)Nc2ccc(F)cc2Cl)CC1C]

A mixture of (mu-1,4-diazabicyclo[2.2.2]octane-kappaN¹:kappaN⁴)(hexamethyl)dialuminium (3.30 g, 12.9 mmol) and 2-chloro-4-fluoroaniline (1.5 ml, 13 mmol) in tetrahydrofuran (34 ml) was stirred for 1 h at 40° C. A solution of methyl 3-methyl-4-({[5-(methylcarbamoyl)-1H-imidazol-4-yl]carbonyl}amino)cyclohexanecarboxylate (830 mg, 2.57 mmol) in tetrahydrofuran (6 ml) was added ant the reaction mixture was stirred for 2 d at room temperature and then for 1 d at 40° C. For work-up, the mixture was poured into a mixture of ice-water and saturated aqueous ammonium chloride solution. Upon extraction of the aqueous phase with a mixture of dichloromethane/2-propanol (4:1) (3×), the combined organic phases were washed with brine, dried over sodium sulfate, filtrated and concentrated under reduced pressure. The residue was purified by flash chromatography (ethyl acetate/methanol gradient, 0%->10% methanol) to give the title compound as mixture of isomers (664 mg).

The mixture of isomers was separated by preparative HPLC [Instrument: Waters Autopurificationsystem SQD; Column: YMC Triart C18 5μ 100×30 mm; eluent A: water+0.1% Vol. trifluoroacetic acid (99%), eluent B: acetonitrile; gradient: 0.00-0.50 min 35% B (25->70 ml/min), 0.51-5.50 min 35-40% B (70 ml/min), temperature: room temperature; DAD scan: 210-400 nm; MS ESI-Pos., scan range 160-1000 m/z] to give 3 fractions: fraction 1 (142 mg, see Example 39), fraction 2 (170 mg, see Example 40) and fraction 3 (93 mg, see Example 41).

Analytical LC-MS method: Instrument: Agilent UHPLC 1290 MS; Column: YMC Triart C18 1.8p 50×2.1 mm; eluent A: water+0.1% Vol. triflouroacetic acid (99%), eluent B: acetonitrile; gradient: 0-4.5 min 25-35% B; flow 0.8 ml/min; temperature: 50° C.; injection: 1 μl; DAD @220, 254 nm; MS ESI-Pos., scan range 160-1000 m/z

### Example 39

### N⁴-{4-[(2-chloro-4-fluorophenyl)carbamoyl]-2-methylcyclohexyl}-N⁵-methyl-1H-imidazole-4,5-dicarboxamide (mixture diastereoismer 1 and diasteromer 2)

Fraction 1 from Example 38: Mixture of diastereomer 1 and diasteromer 2 (51:49, based on UV 220 nM), 142 mg

Analytical LC-MS (method see Example 38 at 220 nm): Rₜ=2.87 min (diasteromer 1); Rₜ=2.91 min (diasteromer 2), ratio: 51:49

¹H-NMR (400 MHz, DMSO-d₆, characteristic signals given for both isomers): δ [ppm]=13.19 (br. s., 1H), 11.43-10.87 (m, 1H), 9.51 (s, 1H), 8.85-8.43 (m, 1H), 7.82-7.80 (m, 1H), 7.62-7.55 (m, 1H), 7.50 (dd, 1H), 7.21 (td, 1H), 4.03-3.93 (m, 0.5H), 2.84-2.78 (m, 3H), 2.27-2.12 (m, 0.5H), 0.99 (d, 1.5H), 0.95-0.82 (m, 1.5H).

### Example 40

### N⁴-{4-[(2-chloro-4-fluorophenyl)carbamoyl]-2-methylcyclohexyl}-N⁵-methyl-1H-imidazole-4,5-dicarboxamide (mixture diastereoismer 1, diasteromer 2 and diasteromer 3)

Fraction 2 from Example 38: mixture of diasteromer 1, diastereomer 2 and diasteromer 3 (22:56:21 based on UV 220 nM), 170 mg

Analytical LC-MS (method see Example 38 at 220 nm): Rₜ=2.87 min (diasteromer 1); Rₜ=2.91 min (diasteromer 2); Rₜ=3.10 min (diasteromer 3), ratio: 22:56:21

### Example 41

### N⁴-{4-[(2-chloro-4-fluorophenyl)carbamoyl]-2-methylcyclohexyl}-N⁵-methyl-1H-imidazole-4,5-dicarboxamide (mixture of diastereoismer 2 and diasteromer 1)

Fraction 3 from Example 38: mixture of diasteromer 2 and diasteromer 3 (7:93, based on UV @ 220 nm): 93 mg

Analytical LC-MS (method see Example 38 at 220 nm): Rₜ=2.91 min (diasteromer 2); Rₜ=3.10 min (diasteromer 3), ratio: 7:93

¹H-NMR (400 MHz, DMSO-d₆, diasteromer 3): δ [ppm]=13.19 (br. s., 1H), 11.45-11.00 (m, 1H), 9.42 (br. s., 1H), 8.74-8.29 (m, 1H), 7.80 (s, 1H), 7.62 (dd, 1H), 7.50 (dd, 1H), 7.21 (td, 1H), 4.17-4.07 (m, 1H), 2.78 (d, 3H), 1.97-1.45 (m, 8H), 0.91 (d, 3H).

### Example 42

### N-{4-[(2-chloro-4-fluorophenyl)carbamoyl]-2-methylcyclohexyl}-5,7-dimethylpyrazolo[1,5-a]pyrimidine-3-carboxamide (mixture of steroisomers)

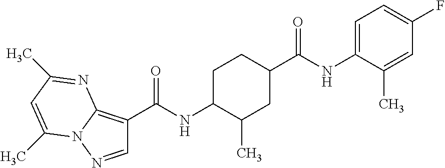 [SMILES: Cc1cc(C)n2ncc(C(=O)NC3CCC(C(=O)Nc4ccc(F)cc4C)CC3C)c2n1]

1-Chloro-N,N,2-trimethylprop-1-en-1-amine (140 mg, 1.04 mmol) was added to a suspension of 4-{[(5,7-dimethylpyrazolo[1,5-a]pyrimidin-3-yl)carbonyl]amino}-3-methylcyclohexanecarboxylic acid (mixture of isomers, 230 mg, 696 μmol) in dichloromethane (30 ml) and the mixture was stirred for 30 min at room temperature. 2-Chloro-4-fluoroaniline (142 mg, 975 μmol) and pyridine (170 μl, 2.1 mmol) were added and the mixture was stirred over night at room temperature. For workup, water was added and the mixture was extracted with dichloromethane. The combined organic phases were washed with saturated sodium bicarbonate solution and brine, filtrated through a phase separator and concentrated. The residue was purified by flash chromatography (25 g Snap Cartdrige, hexanes/ethyl acetate gradient, 20%->100% ethyl acetate) to give the title compound as mixture of isomers 220 mg (95% purity, 66% yield)

LC-MS (Method 2): Rₜ=1.19 min; MS (ESINeg): m/z=456.2 [M−H]⁻

¹H-NMR (400 MHz, DMSO-d6) delta [ppm]: 0.814 (0.40), 0.858 (3.57), 0.875 (3.56), 0.960 (1.18), 0.965 (2.47), 0.981 (2.58), 1.019 (0.89), 1.037 (0.99), 1.054 (3.46), 1.072 (3.56), 1.154 (0.62), 1.172 (1.24), 1.189 (0.64), 1.339 (0.45), 1.341 (0.51), 1.373 (0.56), 1.515 (0.82), 1.548 (1.18), 1.579 (1.03), 1.593 (0.90), 1.616 (1.10), 1.642 (0.82), 1.656 (0.81), 1.682 (1.29), 1.713 (0.87), 1.746 (0.79), 1.773 (1.38), 1.780 (1.38), 1.803 (1.04), 1.806 (1.15), 1.814 (1.21), 1.830 (1.51), 1.840 (1.38), 1.848 (1.34), 1.867 (2.13), 1.930 (0.57), 1.987 (2.22), 2.220 (0.45), 2.238 (0.50), 2.249 (0.42), 2.323 (0.65), 2.327 (0.92), 2.331 (0.67), 2.523 (2.22), 2.574 (0.47), 2.613 (4.32), 2.622 (12.29), 2.624 (16.00), 2.646 (10.41), 2.659 (0.67), 2.665 (0.92), 2.669 (1.09), 2.674 (0.75), 2.678 (0.40), 2.730 (3.56), 2.737 (13.55), 2.743 (11.34), 2.745 (10.70), 2.795 (0.62), 3.310 (0.53), 3.318 (0.75), 4.016 (0.75), 4.034 (0.84), 4.052 (0.53), 4.230 (0.45), 4.258 (0.47), 7.113 (3.28), 7.115 (3.01), 7.121 (1.77), 7.124 (1.66), 7.138 (2.87), 7.141 (2.92), 7.192 (1.03), 7.199 (1.52), 7.206 (0.84), 7.214 (1.60), 7.221 (2.61), 7.228 (1.32), 7.235 (1.15), 7.242 (1.69), 7.248 (0.84), 7.486 (2.41), 7.494 (2.55), 7.501 (0.71), 7.508 (2.45), 7.516 (2.52), 7.523 (0.48), 7.556 (0.40), 7.573 (1.35), 7.583 (1.24), 7.588 (1.96), 7.596 (1.68), 7.605 (1.38), 7.610 (1.71), 7.621 (0.99), 7.626 (0.67), 7.932 (0.65), 7.953 (0.62), 8.236 (1.04), 8.257 (1.04), 8.477 (5.39), 8.482 (1.71), 8.494 (7.60), 8.558 (0.92), 8.582 (0.92), 9.486 (1.20), 9.506 (2.08), 9.523 (0.61), 9.539 (1.66).

### Example 43

### N-{trans-4-[(2-chloro-4-fluorophenyl)carbamoyl]cyclohexyl}pyrazolo[1,5-a]pyrimidine-3-carboxamide

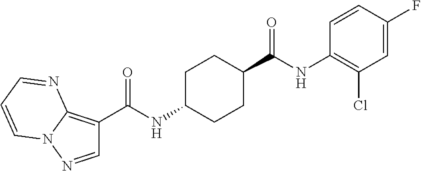 [SMILES: O=C(N[C@H]1CC[C@H](C(=O)Nc2ccc(F)cc2Cl)CC1)c1cnn2cccnc12]

(Benzotriazol-1-yloxy)tripyrrolidinophosphonium hexafluorophosphate (1.05 g, 2.02 mmol) was added to a mixture of pyrazolo[1,5-a]pyrimidine-3-carboxylic acid (299 mg, 1.84 mmol, CAS No. 25940-35-6), trans-4-amino-N-(2-chloro-4-fluorophenyl)cyclohexanecarboxamide hydrochloride (620 mg, 2.02 mmol) and N-ethyl-N-isopropylpropan-2-amine (1.6 ml, 9.2 mmol) in 20 ml N,N-dimethylformamide and the mixture was stirred over night at room temperature. For work-up, the reaction mixture was concentrated under reduced pressure and the residue was stirred with a mixture of water and methanol. The precipitate was collected by filtration, washed with water and methanol and dried to yield the title compound (525 mg, 68% yield).

LC-MS (Method 1): Rₜ=1.01 min; MS (ESIpos) m/z=416.3 [M+H]⁺.

¹H-NMR (400 MHz, DMSO-d₆): δ [ppm]=9.51 (s, 1H), 9.38-9.28 (m, 1H), 8.88-8.76 (m, 1H), 8.58 (s, 1H), 7.85-7.75 (m, 1H), 7.66-7.57 (m, 1H), 7.54-7.43 (m, 1H), 7.32-7.18 (m, 2H), 3.91-3.67 (m, 1H), 2.12-2.01 (m, 2H), 2.01-1.89 (m, 2H), 1.67-1.49 (m, 2H), 1.47-1.27 (m, 2H).

### Example 44

### N-{trans-4-[(2-chloro-4-fluorophenyl)carbamoyl]cyclohexyl}-5,7-dimethylpyrazolo[1,5-a]pyrimidine-3-carboxamide

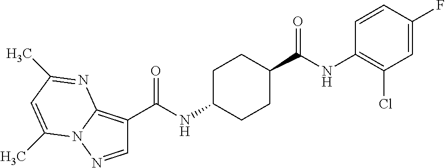 [SMILES: Cc1cc(C)n2ncc(C(=O)N[C@H]3CC[C@H](C(=O)Nc4ccc(F)cc4Cl)CC3)c2n1]

(benzotriazol-1-yloxy)tripyrrolidinophosphonium hexafluorophosphate (678 mg, 1.30 mmol) was added to a mixture of 5,7-dimethyl-pyrazolo[1,5-a]pyrimidine-3-carboxylic acid (226 mg, 1.18 mmol, CAS No. 90349-23-8), trans-4-amino-N-(2-chloro-4-fluorophenyl)cyclohexanecarboxamide hydrochloride (400 mg, 1.32 mmol) and N-ethyl-N-isopropylpropan-2-amine (0.83 ml, 4.7 mmol) in N,N-dimethylformamide (13 ml) and the mixture was stirred over night at room temperature. For work-up, the reaction mixture was concentrated under reduced pressure and the residue was stirred with a mixture of water and methanol. The precipitate was collected by filtration, washed with water and methanol and dried to yield the title compound (290 mg, 54% yield).

LC-MS (Method 1): Rₜ=1.15 min; MS (ESIpos) m/z=444.3 [M+H]⁺.

¹H-NMR (400 MHz, DMSO-d₆): δ [ppm]=9.45 (br. s, 1H), 8.48 (s, 1H), 7.97 (br. d, 1H), 7.60 (dd, 1H), 7.48 (dd, 1H), 7.20 (td, 1H), 7.12-7.09 (m, 1H), 3.84-3.73 (m, 1H), 2.74-2.72 (m, 3H), 2.61 (s, 3H), 2.12-2.02 (m, 2H), 2.00-1.90 (m, 2H), 1.64-1.51 (m, 2H), 1.43-1.28 (m, 2H).

### Example 45

### N-{trans-4-[(2-chloro-4-fluorophenyl)carbamoyl]cyclohexyl}imidazo[1,2-b]pyridazine-3-carboxamide

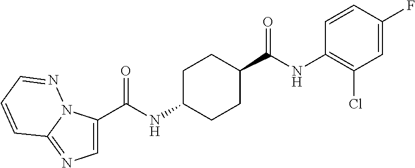 [SMILES: O=C(N[C@H]1CC[C@H](C(=O)Nc2ccc(F)cc2Cl)CC1)c1cnc2cccnn12]

(benzotriazol-1-yloxy)tripyrrolidinophosphonium hexafluorophosphate (83.0 mg, 0.160 mmol) was added to a mixture of imidazo[1,2-b]pyridazine-3-carboxylic acid (23.7 mg, 0.145 mmol, CAS No. 1308384-58-8), trans-4-amino-N-(2-chloro-4-fluorophenyl)cyclohexanecarboxamide hydrochloride (49.0 mg, 0.160 mmol) and N-ethyl-N-isopropylpropan-2-amine (101 μl, 0.58 mmol) in N,N-dimethylformamide (1.6 ml) and the mixture was stirred over night at room temperature. For work-up, the reaction mixture was concentrated under reduced pressure and the residue was stirred with a mixture of water and methanol. The precipitate was collected by filtration, washed with water and methanol and dried to yield the title compound (47.0 mg, 76% yield).

LC-MS (Method 2): Rₜ=0.98 min; MS (ESIpos) m/z=416.3 [M+H]⁺.

¹H-NMR (400 MHz, DMSO-d₆): δ [ppm]=9.51 (s, 1H), 8.84-8.74 (m, 1H), 8.50-8.43 (m, 1H), 8.39-8.32 (m, 1H), 8.30 (s, 1H), 7.65-7.56 (m, 1H), 7.55-7.42 (m, 2H), 7.29-7.14 (m, 1H), 3.96-3.71 (m, 1H), 2.17-2.02 (m, 2H), 2.02-1.84 (m, 2H), 1.69-1.52 (m, 2H), 1.52-1.35 (m, 2H).

### Example 46

### 6-acetyl-N-{trans-4-[(2-chloro-4-fluorophenyl)carbamoyl]cyclohexyl}-7-methylpyrazolo[1,5-a]pyrimidine-3-carboxamide

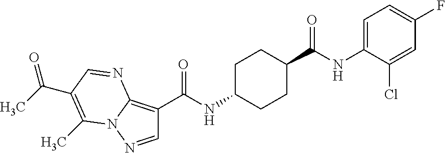 [SMILES: CC(=O)c1cnc2c(C(=O)N[C@H]3CC[C@H](C(=O)Nc4ccc(F)cc4Cl)CC3)cnn2c1C]

(benzotriazol-1-yloxy)tripyrrolidinophosphonium hexafluorophosphate (716 mg, 1.38 mmol) was added to a mixture of 6-acetyl-7-methylpyrazolo[1,5-a]pyrimidine-3-carboxylic acid (274 mg, 1.25 mmol, CAS No. 774183-58-3), trans-4-amino-N-(2-chloro-4-fluorophenyl)cyclohexanecarboxamide hydrochloride (422 mg, 1.38 mmol) and N,N-diisopropylethylamine (1.1 ml, 6.3 mmol) in 14 ml N,N-dimethylformamide and the mixture was stirred over night at room temperature. For work-up, the reaction mixture was concentrated under reduced pressure and the residue was stirred with a mixture of water and methanol. The precipitate was collected by filtration, washed with water and methanol and dried to yield the title compound (440 mg, 69% yield).

LC-MS (Method 1): Rₜ=1.10 min; MS (ESIpos) m/z=472.3 [M+H]⁺.

¹H-NMR (400 MHz, DMSO-d₆): δ [ppm]=9.51 (br. s, 1H), 9.25 (s, 1H), 8.74 (s, 1H), 7.87 (br. d, 1H), 7.60 (dd, 1H), 7.51 (dd, 1H), 7.22 (td, 1H), 3.91-3.76 (m, 1H), 3.07 (s, 3H), 2.74 (s, 3H), 2.15-1.92 (m, 4H), 1.69-1.53 (m, 2H), 1.47-1.31 (m, 2H).

### Example 47

### 6-chloro-N-{trans-4-[(2-chloro-4-fluorophenyl)carbamoyl]cyclohexyl}imidazo[1,2-b]pyridazine-3-carboxamide

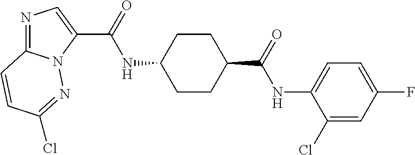 [SMILES: O=C(N[C@H]1CC[C@H](C(=O)Nc2ccc(F)cc2Cl)CC1)c1cnc2ccc(Cl)nn12]

(benzotriazol-1-yloxy)tripyrrolidinophosphonium hexafluorophosphate (1.17 g, 3.30 mmol) was added to a mixture of 6-chloroimidazo[1,2-b]pyridazine-3-carboxylic acid (593 mg, 3.00 mmol, CAS No. 1208084-53-0), trans-4-amino-N-(2-chloro-4-fluorophenyl)cyclohexanecarboxamide hydrochloride (307 mg, 3.30 mmol) and N-ethyl-N-isopropylpropan-2-amine (2.6 ml, 15 mmol) in 33 ml N,N-dimethylformamide and the mixture was stirred over night at room temperature. For work-up, the reaction mixture was concentrated under reduced pressure and the residue was stirred with a mixture of water and methanol. The precipitate was collected by filtration, washed with water and methanol and dried to yield the title compound (815 mg, 60% yield).

LC-MS (Method 1): Rₜ=1.09 min; MS (ESIpos) m/z=550.2 [M+H]⁺.

¹H-NMR (400 MHz, DMSO-d₆): δ [ppm]=9.50 (br. s, 1H), 8.40 (d, 1H), 8.30 (s, 1H), 8.27-8.21 (m, 1H), 7.65-7.57 (m, 2H), 7.51 (dd, 1H), 7.23 (td, 1H), 3.90-3.76 (m, 1H), 2.14-2.02 (m, 2H), 2.02-1.92 (m, 2H), 1.67-1.51 (m, 2H), 1.49-1.35 (m, 2H).

### Example 48

### N-{trans-4-[(2-chloro-4-fluorophenyl)carbamoyl]cyclohexyl}-6-(morpholin-4-yl)imidazo[1,2-b]pyridazine-3-carboxamide

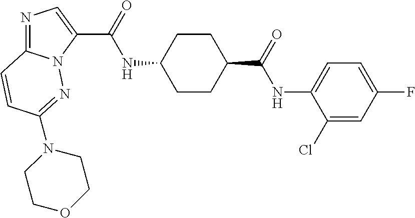 [SMILES: O=C(N[C@H]1CC[C@H](C(=O)Nc2ccc(F)cc2Cl)CC1)c1cnc2ccc(N3CCOCC3)nn12]

A mixture of 6-chloro-N-{trans-4-[(2-chloro-4-fluorophenyl)carbamoyl]cyclohexyl}imidazo[1,2-b]pyridazine-3-carboxamide (100 mg, 0.222 mmol), morpholine (29 μl, 0.33 mmol) and potassium carbonate (46.0 mg, 0.333 mmol) in 2.5 ml dimethylsulfoxide were stirred at 120° C. for 12 h. For workup, the reaction mixture was filtrated and the filtrate was purified by preparative HPLC (Method 9) to yield of the title compound (39.0 mg, 35% yield).

LC-MS (Method 2): Rₜ=1.05 min; MS (ESIpos) m/z=501.3 [M+H]⁺.

¹H-NMR (400 MHz, DMSO-d₆): δ [ppm]=9.50 (br. s, 1H), 8.57 (br. d, 1H), 8.08 (d, 1H), 8.02 (s, 1H), 7.64-7.58 (m, 1H), 7.53-7.48 (m, 1H), 7.42 (d, 1H), 7.26-7.19 (m, 1H), 3.88-3.71 (m, 5H), 3.54 (d, 4H), 2.19-2.07 (m, 2H), 2.03-1.92 (m, 2H), 1.67-1.52 (m, 2H), 1.43-1.27 (m, 2H).

### Example 49

### N-{trans-4-[(2-chloro-4-fluorophenyl)carbamoyl]cyclohexyl}-6-(4-methylpiperazin-1-yl)imidazo[1,2-b]pyridazine-3-carboxamide

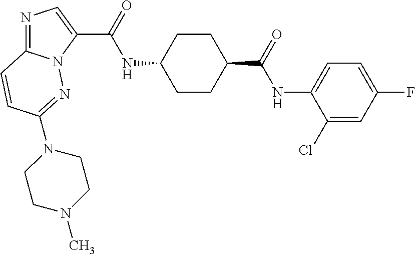 [SMILES: CN1CCN(c2ccc3ncc(C(=O)N[C@H]4CC[C@H](C(=O)Nc5ccc(F)cc5Cl)CC4)n3n2)CC1]

A mixture of 6-chloro-N-{trans-4-[(2-chloro-4-fluorophenyl)carbamoyl]cyclohexyl}imidazo[1,2-b]pyridazine-3-carboxamide (62.0 mg, 0.138 mmol), 1-methylpiperazine (29 μl, 0.21 mmol) and potassium carbonate (28.5 mg, 0.207 mmol) in 1.6 ml dimethylsulfoxide were stirred at 120° C. for 12 h. For workup, the reaction mixture was filtrated and the filtrate was purified by preparative HPLC (Method 9) to yield of the title compound (25.0 mg, 35% yield).

LC-MS (Method 2): Rₜ=1.04 min; MS (ESIpos) m/z=514.4 [M+H]⁺.

¹H-NMR (400 MHz, DMSO-d₆): δ [ppm]=9.49 (br. s, 1H), 8.63 (br. d, 1H), 8.05 (d, 1H), 8.01 (s, 1H), 7.65-7.59 (m, 1H), 7.54-7.48 (m, 1H), 7.43 (d, 1H), 7.26-7.19 (m, 1H), 3.87-3.73 (m, 1H), 3.61-3.49 (m, 4H), 2.26 (s, 3H), 2.20-2.07 (m, 2H), 2.03-1.92 (m, 2H), 1.68-1.51 (m, 2H), 1.41-1.25 (m, 2H).

### Example 50

### N-{trans-4-[(2-chloro-4-fluorophenyl)carbamoyl]cyclohexyl}-6-methoxyimidazo[1,2-b]pyridazine-3-carboxamide

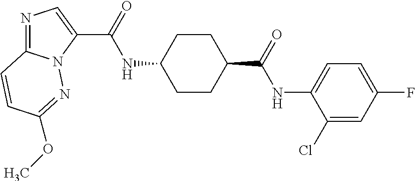 [SMILES: COc1ccc2ncc(C(=O)N[C@H]3CC[C@H](C(=O)Nc4ccc(F)cc4Cl)CC3)n2n1]

(Benzotriazol-1-yloxy)tripyrrolidinophosphonium hexafluorophosphate (226 mg, 0.435 mmol) was added to a mixture of 6-methoxyimidazo[1,2-b]pyridazine-3-carboxylate (85.0 mg, 0.395 mmol), trans-4-amino-N-(2-chloro-4-fluorophenyl)cyclohexanecarboxamide hydrochloride (133 mg, 0.435 mmol) and N-ethyl-N-isopropylpropan-2-amine (0.34 ml, 2.0 mmol) in 4.4 ml N,N-dimethylformamide and the mixture was stirred over night at room temperature. For work-up, the reaction mixture was concentrated under reduced pressure and the residue purified by preparative HPLC [Column: Acquity Xbrigde C18 5 μm 150×50 mm; eluent A: water+0.1 vol % formic acid (99%), Eluent B: acetonitrile; gradient: 0-12 min 25-55% B, flow 150 ml/min; temperature: room temperature; injection: 5×1 ml DMSO-solution] to yield of the title compound (55.5 mg, 31% yield).

LC-MS (Method 1): Rₜ=1.05 min; MS (ESIpos) m/z=446.3 [M+H]⁺.

¹H-NMR (400 MHz, DMSO-d₆): δ [ppm]=9.49 (s, 1H), 8.38 (d, 1H), 8.22 (d, 1H), 8.12 (s, 1H), 7.62 (dd, 1H), 7.51 (dd, 1H), 7.22 (td, 1H), 7.14 (d, 1H), 4.07 (s, 3H), 3.89-3.76 (m, 1H), 2.20-2.08 (m, 2H), 2.03-1.92 (m, 2H), 1.68-1.53 (m, 2H), 1.48-1.32 (m, 2H).

### Example 51

### N-{trans-4-[(2-chloro-4-fluorophenyl)carbamoyl]cyclohexyl}-6-(pyrrolidin-1-yl)imidazo[1,2-b]pyridazine-3-carboxamide

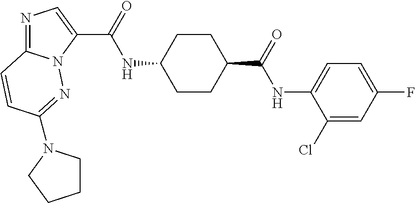 [SMILES: O=C(N[C@H]1CC[C@H](C(=O)Nc2ccc(F)cc2Cl)CC1)c1cnc2ccc(N3CCCC3)nn12]

A mixture of 6-chloro-N-{trans-4-[(2-chloro-4-fluorophenyl)carbamoyl]cyclohexyl}imidazo[1,2-b]pyridazine-3-carboxamide (100 mg 0.222 mmol), pyrrolidine (28 μl, 0.33 mmol) and potassium carbonate (46.0 mg, 0.333 mmol) in 2.5 ml dimethylsulfoxide were stirred at 120° C. for 12 h. For workup, the reaction mixture was filtrated and the filtrate was directly purified by preparative HPLC (Method 9). The isolated product was recrystallized from ethanol to yield of the title compound (36.0 mg, 32% yield).

LC-MS (Method 1): Rₜ=1.11 min; MS (ESIpos) m/z=485.2 [M+H]⁺.

¹H-NMR (400 MHz, DMSO-d₆): δ [ppm]=9.50 (br. s, 1H), 8.92 (br. d, 1H), 8.00 (d, 1H), 7.95 (s, 1H), 7.66-7.59 (m, 1H), 7.54-7.47 (m, 1H), 7.27-7.18 (m, 1H), 7.05 (d, 1H), 3.88-3.71 (m, 1H), 3.56-3.46 (m, 4H), 2.20-2.09 (m, 2H), 2.04 (s, 6H), 1.67-1.53 (m, 2H), 1.41-1.26 (m, 2H).

### Example 52

### N-{trans-4-[(2-chloro-4-fluorophenyl)carbamoyl]cyclohexyl}-6-(dimethylamino)imidazo[1,2-b]pyridazine-3-carboxamide

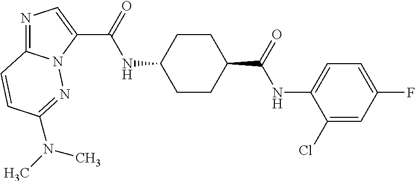 [SMILES: CN(C)c1ccc2ncc(C(=O)N[C@H]3CC[C@H](C(=O)Nc4ccc(F)cc4Cl)CC3)n2n1]

A mixture of 6-chloro-N-{trans-4-[(2-chloro-4-fluorophenyl)carbamoyl]cyclohexyl}imidazo[1,2-b]pyridazine-3-carboxamide (120 mg, 0.266 mmol), dimethylamine hydrochloride (65.2 mg, 0.799 mmol) and potassium carbonate (184 mg, 1.33 mmol) in 3.0 ml dimethylsulfoxide were stirred at 120° C. for 12 h. For workup, the reaction mixture was filtrated and the filtrate was purified by preparative HPLC (Method 9). The obtained product was recrystallized from ethanol to yield of the title compound (16.5 mg, 13% yield).

LC-MS (Method 1): Rₜ=1.02 min; MS (ESIpos) m/z=459.3 [M+H]⁺.

¹H-NMR (400 MHz, DMSO-d₆): δ [ppm]=9.49 (br. s, 1H), 8.83 (br. d, 1H), 8.01 (d, 1H), 7.97 (s, 1H), 7.66-7.59 (m, 1H), 7.53-7.46 (m, 1H), 7.29-7.17 (m, 2H), 3.88-3.73 (m, 1H), 3.16 (s, 6H), 2.21-2.09 (m, 2H), 2.02-1.90 (m, 2H), 1.67-1.53 (m, 2H), 1.40-1.25 (m, 2H).

### Example 53

### N-{trans-4-[(2-chloro-4-fluorophenyl)carbamoyl]cyclohexyl}-6-(methylamino)imidazo[1,2-b]pyridazine-3-carboxamide

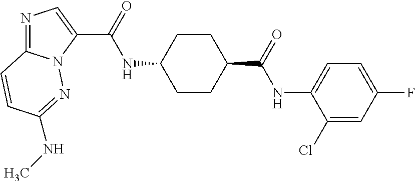 [SMILES: CNc1ccc2ncc(C(=O)N[C@H]3CC[C@H](C(=O)Nc4ccc(F)cc4Cl)CC3)n2n1]

A mixture of 6-chloro-N-{trans-4-[(2-chloro-4-fluorophenyl)carbamoyl]cyclohexyl}imidazo[1,2-b]pyridazine-3-carboxamide (120 mg, 0.266 mmol), methanamine (0.40 ml, 0.80 mmol, 2 M solution in tetrahydrofuran) and potassium carbonate (55.2 mg, 0.400 mmol) in 3.0 ml dimethylsulfoxide were stirred at 120° C. for 12 h. For workup, the reaction mixture was filtrated and the filtrate was purified by preparative HPLC (Method 9) to yield the title compound (10.8 mg, 90% purity, 8% yield).

LC-MS (Method 1): Rₜ=0.95 min; MS (ESIpos) m/z=445.3 [M+H]⁺.

¹H-NMR (400 MHz, DMSO-d₆): δ [ppm]=9.49 (br. s, 1H), 8.96 (br. d, 1H), 7.92-7.84 (m, 2H), 7.70-7.46 (m, 3H), 7.27-7.18 (m, 1H), 6.83 (d, 1H), 3.86-3.74 (m, 1H), 2.87 (d, 3H), 2.24-2.09 (m, 2H), 2.02-1.90 (m, 2H), 1.68-1.52 (m, 2H), 1.42-1.24 (m, 2H).

### Example 54

### N-{trans-4-[(2-chloro-4-fluorophenyl)carbamoyl]cyclohexyl}-7-methyl-6-[1-(methylamino)ethyl]pyrazolo[1,5-a]pyrimidine-3-carboxamide formic acid salt

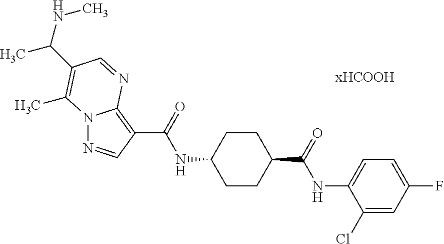 [SMILES: CNC(C)c1cnc2c(C(=O)N[C@H]3CC[C@H](C(=O)Nc4ccc(F)cc4Cl)CC3)cnn2c1C]

Titanium(IV) isopropoxide (146 μl, 0.487 mmol) was added to a mixture of 6-acetyl-N-{trans-4-[(2-chloro-4-fluorophenyl)carbamoyl]cyclohexyl}-7-methylpyrazolo[1,5-a]pyrimidine-3-carboxamide (115 mg, 0.244 mmol) and methanamine (244 μl, 0.49 mmol, 2 M solution in tetrahydrofuran) in 1.2 ml tetrahydrofuran and the mixture was stirred for 24 h at room temperature. Sodium cyanoborohydride (38.0 mg, 0.604 mmol) was added and the mixture was stirred for 12 h at room temperature. For work-up, the reaction mixture was concentrated and the residue was purified by preparative HPLC (Method 8) to yield the title compound (20.0 mg, 15% yield).

LC-MS (Method 1): Rₜ=0.86 min; MS (ESIpos) m/z=487.3 [M+H]⁺.

¹H-NMR (400 MHz, DMSO-d₆): δ [ppm]=9.52 (br. s, 1H), 8.85 (s, 1H), 8.55 (s, 1H), 8.21 (br. s, 1H), 7.88 (br. d, 1H), 7.64-7.57 (m, 1H), 7.54-7.47 (m, 1H), 7.27-7.18 (m, 1H), 4.05-3.96 (m, 1H), 3.89-3.75 (m, 1H), 2.84 (s, 3H), 2.18 (s, 3H), 2.12-2.03 (m, 2H), 2.01-1.93 (m, 2H), 1.65-1.51 (m, 2H), 1.44-1.30 (m, 5H).

### Example 55

### N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide formic acid salt

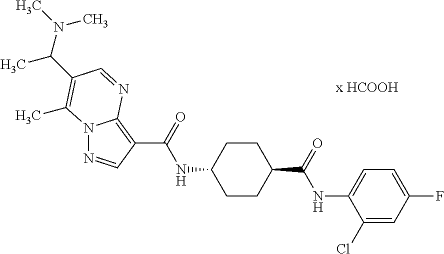 [SMILES: Cc1c(C(C)N(C)C)cnc2c(C(=O)N[C@H]3CC[C@H](C(=O)Nc4ccc(F)cc4Cl)CC3)cnn12]

Titanium(IV) isopropoxide (146 μl, 0.487 mmol) was added to a mixture of 6-acetyl-N-{trans-4-[(2-chloro-4-fluorophenyl)carbamoyl]cyclohexyl}-7-methylpyrazolo[1,5-a]pyrimidine-3-carboxamide (115 mg, 0.244 mmol) and dimethyl amine (244 μl, 0.49 mmol, 2 M solution in tetrahydrofuran) in 1.2 ml tetrahydrofuran and the mixture was stirred for 24 h at room temperature. Sodium cyanoborohydride (38.0 mg, 0.604 mmol) was added and the mixture was stirred for 12 h at room temperature. For work-up, the reaction mixture was concentrated and the residue was purified by preparative HPLC (Method 8) to yield the title compound (39 mg, 29% yield).

LC-MS (Method 1): Rₜ=0.86 min; MS (ESIpos) m/z=501.3 [M+H]⁺.

¹H-NMR (400 MHz, DMSO-d₆): δ [ppm]=9.51 (br. s, 1H), 8.79 (s, 1H), 8.56 (s, 1H), 8.15 (s, 1H), 7.87 (br. d, 1H), 7.63-7.58 (m, 1H), 7.54-7.48 (m, 1H), 7.27-7.18 (m, 1H), 3.90-3.75 (m, 1H), 3.70-3.61 (m, 1H), 2.86 (s, 3H), 2.19 (s, 6H), 2.11-2.02 (m, 2H), 2.01-1.92 (m, 2H), 1.67-1.51 (m, 2H), 1.44-1.29 (m, 5H).

N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide formic acid salt (60.0 mg) was separated into both enantiomers by chiral preparative HPLC [Instrument: Labomatic pump HD-5000, Labomatic SP-3000, Labocord 5000, Labomatic Labcol Vario 4000, Gilson GX-241; Column: Chiralpak IA 5 μm 250×30 mm Nr.029; solvent: methyl tert-butyl ether/ethanol/diethylamine 90:10:0.1 (v/v/v/v); flow: 40 ml/min; temperature: room temperature; MWD 254 nm;] to yield N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide (entantiomer 1) (13.5 mg, Example 56) and enantiomer 2 (15.7 mg Example 57).

Chrial analytical HPLC method: Instrument: Agilent: 1260, MWD, column: Chiralpak IA 5 μm 100×4.6 mm; solvent: methyl tert-buthyl ether/ethanol/diethylamine 90:10:0.1; flow 1.0 ml/min; temperature: room temperature; injection: 5 μl; DAD: 254 nm; solution: 1.0 mg/ml methanol

### Example 56

### N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide enantiomer 1

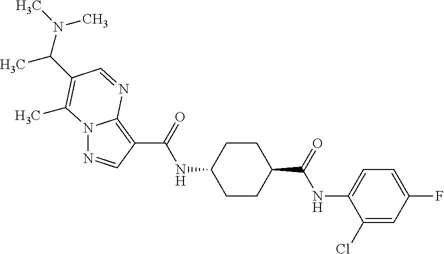 [SMILES: Cc1c(C(C)N(C)C)cnc2c(C(=O)N[C@H]3CC[C@H](C(=O)Nc4ccc(F)cc4Cl)CC3)cnn12]

Chiral analytical HPLC (method see Example 55): Rₜ=5.25 min, purity 99.9% by UV

¹H-NMR (400 MHz, DMSO-d₆): δ [ppm]=9.52 (s, 1H), 8.78 (s, 1H), 8.55 (s, 1H), 7.87 (d, 1H), 7.59 (dd, 1H), 7.50 (dd, 1H), 7.27-7.18 (m, 1H), 3.89-3.74 (m, 1H), 3.64 (q, 1H), 2.85 (s, 3H), 2.18 (s, 6H), 2.11-2.02 (m, 2H), 2.00-1.90 (m, 2H), 1.65-1.51 (m, 2H), 1.44-1.30 (m, 5H).

### Example 57

### N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide enantiomer 2

Chiral analytical HPLC (method see Example 55): Rₜ=6.58 min, purity 99.9% by UV

### Example 58

### N-{trans-4-[(2-chloro-4-fluorophenyl)carbamoyl]cyclohexyl}-6-(1-hydroxyethyl)-7-methylpyrazolo[1,5-a]pyrimidine-3-carboxamide

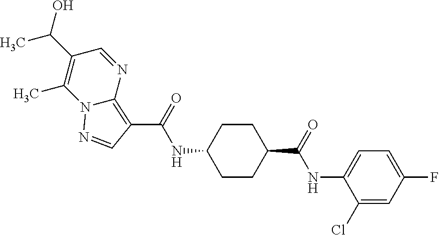 [SMILES: Cc1c(C(C)O)cnc2c(C(=O)N[C@H]3CC[C@H](C(=O)Nc4ccc(F)cc4Cl)CC3)cnn12]

Titanium(IV) isopropoxide (127 μl, 0.424 mmol) was added to a mixture of 6-acetyl-N-{trans-4-[(2-chloro-4-fluorophenyl)carbamoyl]cyclohexyl}-7-methylpyrazolo[1,5-a]pyrimidine-3-carboxamide (100 mg, 0.212 mmol) and morpholine (22 μl, 0.25 mmol) in 1.0 ml tetrahydrofuran and the mixture was stirred for 24 h at room temperature. Sodium cyanoborohydride (33.0 mg, 0.525 mmol) was added and the mixture was stirred for 12 h at room temperature. For work-up, the reaction mixture was concentrated and the residue was purified by preparative HPLC (Method 8). The obtained product was recrystallized from ethanol to yield of the title compound (34.5 mg, 34% yield).

LC-MS (Method 2): Rₜ=0.95 min; MS (ESIpos) m/z=474.3 [M+H]⁺.

¹H-NMR (400 MHz, DMSO-d₆): δ [ppm]=9.92-9.74 (m, 1H), 9.51 (br. s, 1H), 8.04-7.79 (m, 2H), 7.60 (dd, 1H), 7.53-7.46 (m, 2H), 7.22 (td, 1H), 5.27-5.20 (m, 1H), 3.80-3.65 (m, 1H), 2.46-2.38 (m, 1H), 2.22 (s, 3H), 2.00-1.86 (m, 4H), 1.63-1.42 (m, 2H), 1.42-1.27 (m, 5H).

### Example 59

### 2-amino-N-{trans-4-[(2-chloro-4-fluorophenyl)carbamoyl]cyclohexyl}pyrazolo[1,5-a]pyrimidine-3-carboxamide

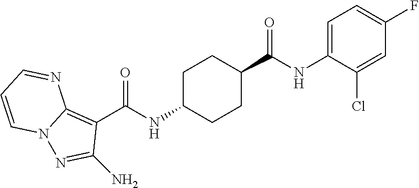 [SMILES: Nc1nn2cccnc2c1C(=O)N[C@H]1CC[C@H](C(=O)Nc2ccc(F)cc2Cl)CC1]

(Benzotriazol-1-yloxy)tripyrrolidinophosphonium hexafluorophosphate (220 mg, 422 μmol) was added to a mixture of 2-aminopyrazolo[1,5-a]pyrimidine-3-carboxylic acid lithium/and or sodium salt (86.2 mg, 82% purity, 384 μmol), trans-4-amino-N-(2-chloro-4-fluorophenyl)cyclohexanecarboxamide hydrochloric acid salt (130 mg, 422 μmol) and N-ethyl-N-isopropylpropan-2-amine (330 μl, 1.9 mmol) in DMF (4.3 ml) and the mixture was stirred over night at room temperature. For work-up, the reaction mixture was concentrated under reduced pressure and the residue purified by preparative HPLC (Method 9) to give the title compound (18.0 m, 11% yield).

LC-MS (Method 2): Rₜ=1.04 min; MS (ESIpos): m/z=431.3 [M+H]⁺

¹H-NMR (400 MHz, DMSO-d₆): δ [ppm]=9.51 (s, 1H), 8.91 (dd, 1H), 8.50 (dd, 1H), 7.62-7.44 (m, 3H), 7.21 (td, 1H), 6.98 (dd, 1H), 6.52 (s, 2H), 3.86-3.68 (m, 1H), 2.13-1.86 (m, 4H), 1.66-1.48 (m, 2H), 1.42-1.27 (m, 2H).

### Example 60

### 7-tert-butyl-N-{trans-4-[(2-chloro-4-fluorophenyl)carbamoyl]cyclohexyl}-5-methylpyrazolo[1,5-a]pyrimidine-3-carboxamide

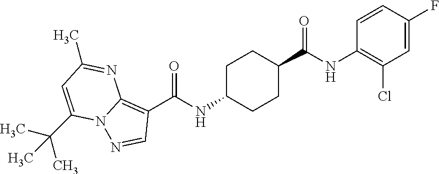 [SMILES: Cc1cc(C(C)(C)C)n2ncc(C(=O)N[C@H]3CC[C@H](C(=O)Nc4ccc(F)cc4Cl)CC3)c2n1]

Was prepared in analogy to the synthesis of 2-amino-N-{trans-4-[(2-chloro-4-fluorophenyl)carbamoyl]cyclohexyl}pyrazolo[1,5-a]pyrimidine-3-carboxamide using 7-tert-butyl-5-methylpyrazolo[1,5-a]pyrimidine-3-carboxylic acid (55.2 mg, 237 μmol) as starting material. For work-up, water was added and the precipitate formed was collected by filtration and washed with methanol and dried. The crude product was recrystallized from methanol to provide the title compound (73.0 mg, 63% yield).

LC-MS (Method 1): Rₜ=1.38 min; MS (ESIpos): m/z=486.2 [M+H]⁺

¹H-NMR (400 MHz, DMSO-d₆): δ [ppm]=9.47 (s, 1H), 8.52 (s, 1H), 8.08 (d, 1H), 7.61 (dd, 1H), 7.50 (dd, 1H), 7.26-7.18 (m, 1H), 7.05 (s, 1H), 3.87-3.71 (m, 1H), 2.66 (s, 3H), 2.13-2.03 (m, 2H), 2.01-1.88 (m, 2H), 1.66-1.47 (m, 11H), 1.45-1.27 (m, 2H).

### Example 61

### N-{trans-4-[(2-chloro-4-fluorophenyl)carbamoyl]cyclohexyl}-6-methylpyrazolo[1,5-a]pyrimidine-3-carboxamide

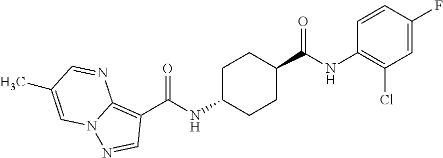 [SMILES: Cc1cnc2c(C(=O)N[C@H]3CC[C@H](C(=O)Nc4ccc(F)cc4Cl)CC3)cnn2c1]

Was prepared in analogy to the synthesis of 2-amino-N-{trans-4-[(2-chloro-4-fluorophenyl)carbamoyl]cyclohexyl}pyrazolo[1,5-a]pyrimidine-3-carboxamide using 6-methylpyrazolo[1,5-a]pyrimidine-3-carboxylic acid (42.0 mg, 237 μmol, CAS No. 869941-96-8) as starting material. For work-up, the reaction mixture was concentrated and the residue was stirred with methanol. The precipitate formed was collected by filtration and washed with methanol and dried to give the title compound (77.6 mg, 73% yield).

LC-MS (Method 1): Rₜ=1.06 min; MS (ESIpos): m/z=430 [M+H]⁺

¹H-NMR (400 MHz, DMSO-d6) delta [ppm]: 1.329 (0.60), 1.353 (1.53), 1.357 (1.64), 1.363 (1.58), 1.383 (1.75), 1.388 (1.91), 1.392 (1.86), 1.414 (0.87), 1.417 (0.87), 1.423 (0.76), 1.528 (0.66), 1.535 (0.82), 1.560 (1.86), 1.565 (1.91), 1.569 (1.75), 1.591 (1.75), 1.597 (1.86), 1.599 (1.86), 1.625 (0.71), 1.632 (0.66), 1.936 (2.18), 1.940 (2.18), 1.970 (1.80), 1.975 (1.58), 2.033 (1.86), 2.037 (1.97), 2.043 (2.08), 2.065 (1.97), 2.069 (1.86), 2.075 (1.75), 2.322 (0.49), 2.327 (0.66), 2.332 (0.55), 2.377 (15.78), 2.379 (16.00), 2.439 (0.87), 2.460 (1.20), 2.468 (1.86), 2.523 (4.10), 2.665 (0.49), 2.669 (0.60), 2.674 (0.44), 3.158 (1.09), 3.171 (1.09), 3.299 (0.60), 3.411 (0.60), 3.779 (0.44), 3.789 (0.76), 3.798 (0.93), 3.808 (0.82), 3.818 (0.93), 3.828 (0.76), 7.190 (1.04), 7.197 (1.15), 7.211 (1.86), 7.219 (1.97), 7.233 (1.20), 7.240 (1.20), 7.484 (2.08), 7.491 (2.18), 7.506 (2.18), 7.513 (2.08), 7.572 (2.02), 7.587 (2.18), 7.594 (2.02), 7.609 (1.80), 7.738 (2.35), 7.757 (2.35), 8.487 (9.17), 8.727 (4.31), 8.732 (4.31), 9.169 (2.84), 9.172 (4.04), 9.178 (2.84), 9.513 (4.15).

### Example 62

### N-{trans-4-[(2-chloro-4-fluorophenyl)carbamoyl]cyclohexyl}-5-cyclopropyl-7-methylpyrazolo[1,5-a]pyrimidine-3-carboxamide

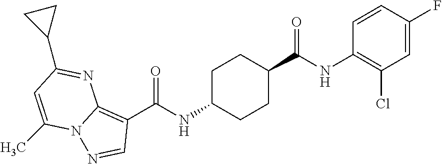 [SMILES: Cc1cc(C2CC2)nc2c(C(=O)N[C@H]3CC[C@H](C(=O)Nc4ccc(F)cc4Cl)CC3)cnn12]

Was prepared in analogy to the synthesis of 2-amino-N-{trans-4-[(2-chloro-4-fluorophenyl)carbamoyl]cyclohexyl}pyrazolo[1,5-a]pyrimidine-3-carboxamide using 5-cyclopropyl-7-methylpyrazolo[1,5-a]pyrimidine-3-carboxylic acid (42.0 mg, 194 μmol) as starting material. For work-up, the reaction mixture was concentrated and the residue was stirred with methanol. The precipitate formed was collected by filtration and dried to give the title compound (48.0 mg, 51% yield).

LC-MS (Method 1): Rₜ=1.21 min; MS (ESIpos): m/z=470.3 [M+H]⁺

¹H-NMR (400 MHz, DMSO-d₆): δ [ppm]=9.49 (s, 1H), 8.45 (s, 1H), 7.96 (d, 1H), 7.63 (dd, 1H), 7.50 (dd, 1H), 7.26-7.16 (m, 2H), 3.81-3.69 (m, 1H), 2.73 (s, 3H), 2.31-2.23 (m, 1H), 2.16-2.06 (m, 2H), 1.99-1.90 (m, 2H), 1.68-1.50 (m, 2H), 1.37-1.18 (m, 4H), 1.15-1.08 (m, 2H).

### Example 63

### N⁵-{trans-4-[(2-chloro-4-fluoro-5-methylphenyl)carbamoyl]cyclohexyl}-N⁴-methyl-1H-imidazole-4,5-dicarboxamide

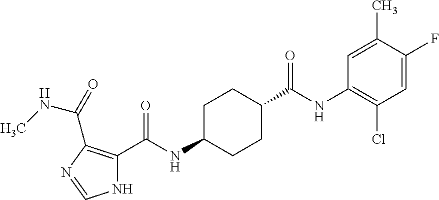 [SMILES: CNC(=O)c1nc[nH]c1C(=O)N[C@H]1CC[C@H](C(=O)Nc2cc(C)c(F)cc2Cl)CC1]

A mixture of phenyl 5-({trans-4-[(2-chloro-4-fluoro-5-methylphenyl)carbamoyl]cyclohexyl}carbamoyl)-1H-imidazole-4-carboxylate (100 mg, 0.200 mmol) and methanamine (0.50 ml, 1.0 mmol, 2 M solution in tetrahydrofuran) in tetrahydrofuran (5.0 ml) was stirred at 60° C. over night. For work-up the reaction mixture was concentrated and the crude product was stirred with methanol. The precipitate was collected by filtration, washed with methanol and dried to yield the title compound (52.0 mg, 60% yield).

LC-MS (Method 6): Rₜ=1.02 min; MS (ESIpos) m/z=436.2 [M+H]⁺.

¹H-NMR (500 MHz, DMSO-d₆): δ [ppm]=13.61-12.72 (m, 1H), 11.53-10.66 (m, 1H), 9.45 (s, 1H), 8.32 (s, 1H), 7.80 (s, 1H), 7.49 (d, 1H), 7.40 (d, 1H), 3.83-3.62 (m, 1H), 2.82 (d, 3H), 2.48-2.39 (m, 1H), 2.21 (d, 3H), 2.12-1.87 (m, 4H), 1.65-1.17 (m, 4H).

### Example 64

### N⁵-{trans-4-[(2-chloro-4-fluoro-5-methylphenyl)carbamoyl]cyclohexyl}-N⁴-(tetrahydrofuran-2-ylmethyl)-1H-imidazole-4,5-dicarboxamide

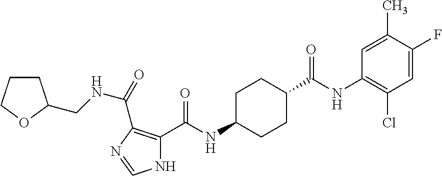 [SMILES: Cc1cc(NC(=O)[C@H]2CC[C@H](NC(=O)c3[nH]cnc3C(=O)NCC3CCCO3)CC2)c(Cl)cc1F]

A mixture of phenyl 5-({trans-4-[(2-chloro-4-fluoro-5-methylphenyl)carbamoyl]cyclohexyl}carbamoyl)-1H-imidazole-4-carboxylate (113 mg, 0.226 mmol) and 1-(tetrahydrofuran-2-yl)methanamine (115 mg, 1.13 mmol) in tetrahydrofuran (5.7 ml) was stirred at 60° C. over night. The precipitate was collected by filtration, washed with methanol and dried to yield the title compound (47.0 mg, 41% yield).

LC-MS (Method 6): Rₜ=0.1.11 min; MS (ESIpos) m/z=506.2 [M+H]⁺.

¹H-NMR (400 MHz, DMSO-d₆): δ [ppm]=13.22 (br. s., 1H), 11.41-10.93 (m, 1H), 9.46 (s, 1H), 8.66-8.15 (m, 1H), 7.82 (s, 1H), 7.50 (d, 1H), 7.41 (d, 1H), 4.03-3.90 (m, 1H), 3.84-3.68 (m, 2H), 3.68-3.59 (m, 1H), 3.44-3.35 (m, 2H), 2.50-2.38 (m, 1H), 2.21 (d, 3H), 2.13-1.68 (m, 8H), 1.68-1.19 (m, 4H).

### Example 65

### N⁵-{trans-4-[(2-chloro-4,6-difluorophenyl)carbamoyl]cyclohexyl}-N⁴-(1-methoxypropan-2-yl)-1H-imidazole-4,5-dicarboxamide

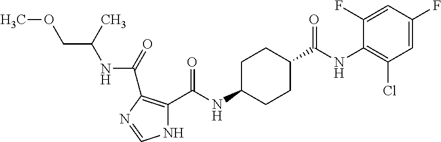 [SMILES: COCC(C)NC(=O)c1nc[nH]c1C(=O)N[C@H]1CC[C@H](C(=O)Nc2c(F)cc(F)cc2Cl)CC1]

A mixture of phenyl 5-({trans-4-[(2-chloro-4,6-difluorophenyl)carbamoyl]cyclohexyl}carbamoyl)-1H-imidazole-4-carboxylate (200 mg, 93% purity, 0.370 mmol) and 1-methoxypropan-2-amine (1.65 mg, 1.85 mmol, CAS No 37143-54-7) in tetrahydrofuran (5.7 ml) was stirred at room temperature over night. For work-up, the reaction mixture was concentrated and the residue was purified by flash chromatography (25 g Snap Cartridge, hexanes/ethyl acetate gradient, 50%->100% ethyl acetate) to yield the title compound (79.0 mg, 42% yield).

LC-MS (Method 2): Rₜ=0.98 min; MS (ESIpos) m/z=498.3 [M+H]⁺.

¹H-NMR (400 MHz, DMSO-d₆): δ [ppm]=13.20-13.02 (m, 1H), 11.24-10.89 (m, 1H), 9.57 (s, 1H), 8.46-8.12 (m, 1H), 7.79 (s, 1H), 7.47-7.34 (m, 2H), 4.26-4.01 (m, 1H), 3.88-3.61 (m, 1H), 3.45-3.36 (m, 1H), 3.36-3.31 (m, 1H), 3.27 (s, 3H), 2.46-2.34 (m, 1H), 2.14-1.80 (m, 4H), 1.60-1.25 (m, 4H), 1.15 (d, 3H).

### Example 66

### N⁵-{trans-4-[(2-chloro-4,6-difluorophenyl)carbamoyl]cyclohexyl}-N⁴-[(2S)-1-methoxypropan-2-yl]-1H-imidazole-4,5-dicarboxamide

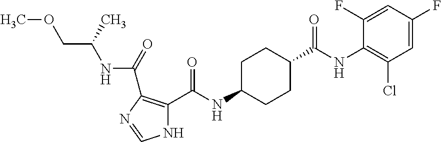 [SMILES: COC[C@H](C)NC(=O)c1nc[nH]c1C(=O)N[C@H]1CC[C@H](C(=O)Nc2c(F)cc(F)cc2Cl)CC1]

Was prepared in analogy to the synthesis of N⁵-{trans-4-[(2-chloro-4,6-difluorophenyl)carbamoyl]cyclohexyl}-N⁴-(1-methoxypropan-2-yl)-1H-imidazole-4,5-dicarboxamide using (2S)-(+)-1-methoxypropan-2-amine [CAS No: 99636-32-5] as starting material and purified by preparative HPLC (Method 9) to give the title compound.

Specific optical rotation (Method 11): +1.9 (c=1.0 g/100 ml in DMSO)

### Example 67

### N⁵-{trans-4-[(2-chloro-4,6-difluorophenyl)carbamoyl]cyclohexyl}-N⁴-[(2R)-1-methoxypropan-2-yl]-1H-imidazole-4,5-dicarboxamide

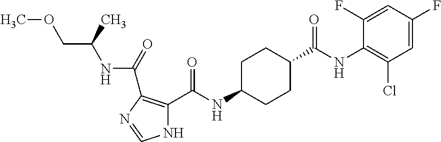 [SMILES: COC[C@@H](C)NC(=O)c1nc[nH]c1C(=O)N[C@H]1CC[C@H](C(=O)Nc2c(F)cc(F)cc2Cl)CC1]

Was prepared in analogy to the synthesis of N⁵-{trans-4-[(2-chloro-4,6-difluorophenyl)carbamoyl]cyclohexyl}-N⁴-(1-methoxypropan-2-yl)-1H-imidazole-4,5-dicarboxamide using (2R)-(−)1-methoxypropan-2-amine hydrochloride (93 mg, 0.74 mmol) as starting material and triethylamine (103 μl) as external base.

Specific optical rotation (Method 11): −3.1 (c=1.0 g/100 ml in DMSO)

### Example 68

### N⁵-{trans-4-[(2-chloro-4,6-difluorophenyl)carbamoyl]cyclohexyl}-N⁴-(2-methoxypropyl)-1H-imidazole-4,5-dicarboxamide

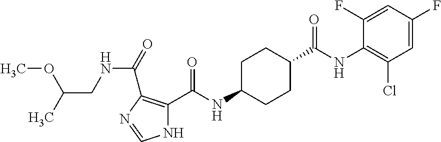 [SMILES: COC(C)CNC(=O)c1nc[nH]c1C(=O)N[C@H]1CC[C@H](C(=O)Nc2c(F)cc(F)cc2Cl)CC1]

A mixture of phenyl 5-({trans-4-[(2-chloro-4,6-difluorophenyl)carbamoyl]cyclohexyl}carbamoyl)-1H-imidazole-4-carboxylate (125 mg, 0.249 mmol), 2-methoxypropan-1-amine hydrochloride (156 mg, 1.24 mmol, CAS No 70807-90-8) and triethylamine (173 μl, 1.24 mmol) in tetrahydrofuran (3.8 ml) was stirred at room temperature over night. The precipitate was collected by filtration, washed with methanol and dried to yield the title compound (54.0 mg, 43% yield).

LC-MS (Method 6): Rₜ=0.97 min; MS (ESIpos) m/z=498 [M+H]⁺.

¹H-NMR (400 MHz, DMSO-d₆): δ [ppm]=13.21 (br. s., 1H), 11.33-10.88 (m, 1H), 9.60 (s, 1H), 8.38 (br. s, 1H), 7.82 (s, 1H), 7.50-7.36 (m, 2H), 3.93-3.64 (m, 1H), 3.63-3.36 (m, 2H), 3.28 (s, 3H), 2.16-1.72 (m, 4H), 1.67-1.18 (m, 4H), 1.11 (d, 3H).

### Example 69

### N⁵-{trans-4-[(2-chloro-4,6-difluorophenyl)carbamoyl]cyclohexyl}-N⁴-(tetrahydrofuran-2-ylmethyl)-1H-imidazole-4,5-dicarboxamide

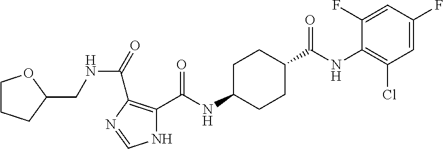 [SMILES: O=C(NCC1CCCO1)c1nc[nH]c1C(=O)N[C@H]1CC[C@H](C(=O)Nc2c(F)cc(F)cc2Cl)CC1]

A mixture of phenyl 5-({trans-4-[(2-chloro-4,6-difluorophenyl)carbamoyl]cyclohexyl}carbamoyl)-1H-imidazole-4-carboxylate (125 mg, 93% purity, 0.231 mmol) and 1-(tetrahydrofuran-2-yl)methanamine (117 mg, 1.16 mmol, CAS No 4795-29-3) in tetrahydrofuran (3.6 ml) was stirred at room temperature over night. The precipitate was collected by filtration, washed with methanol and dried to yield the title compound (53.0 mg, 45% yield).

LC-MS (Method 6): Rₜ=0.97 min; MS (ESIpos) m/z=510.1 [M+H]⁺.

¹H-NMR (400 MHz, DMSO-d₆): δ [ppm]=13.13 (br. s, 1H), 11.41-10.78 (m, 1H), 9.57 (s, 1H), 8.60-8.06 (m, 1H), 7.80 (s, 1H), 7.50-7.31 (m, 2H), 4.01-3.89 (m, 1H), 3.84-3.55 (m, 3H), 3.42-3.32 (m, 2H), 2.45-2.33 (m, 1H), 2.16-1.16 (m 12H).

### Example 70

### N⁵-{trans-4-[(2-chloro-4,6-difluorophenyl)carbamoyl]cyclohexyl}-N⁴-[(2R)-tetrahydrofuran-2-ylmethyl]-1H-imidazole-4,5-dicarboxamide

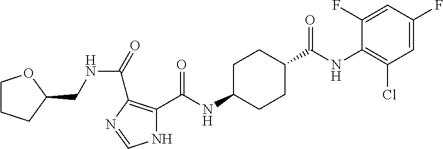 [SMILES: O=C(NC[C@H]1CCCO1)c1nc[nH]c1C(=O)N[C@H]1CC[C@H](C(=O)Nc2c(F)cc(F)cc2Cl)CC1]

Was prepared in analogy to the synthesis of N⁵-{trans-4-[(2-chloro-4,6-difluorophenyl)carbamoyl]cyclohexyl}-N⁴-(tetrahydrofuran-2-ylmethyl)-1H-imidazole-4,5-dicarboxamide using (R)-(−)-1-(tetrahydrofuran-2-yl)methanamine (117 mg, 1.26 mmol, Cas No: 7202-43-9) as starting material.

Specific optical rotation (Method 11): −9.6 (c=1.0 g/100 ml in DMSO)

### Example 71

### N⁵-{trans-4-[(2-chloro-4,6-difluorophenyl)carbamoyl]cyclohexyl}-N⁴-[(2R)-tetrahydrofuran-2-ylmethyl]-1H-imidazole-4,5-dicarboxamide

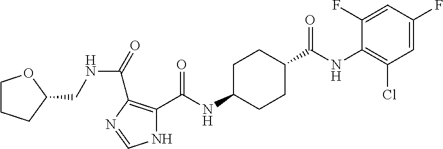 [SMILES: O=C(NC[C@@H]1CCCO1)c1nc[nH]c1C(=O)N[C@H]1CC[C@H](C(=O)Nc2c(F)cc(F)cc2Cl)CC1]

Was prepared in analogy to the synthesis of N⁵-{trans-4-[(2-chloro-4,6-difluorophenyl)carbamoyl]cyclohexyl}-N⁴-(tetrahydrofuran-2-ylmethyl)-1H-imidazole-4,5-dicarboxamide using (S)-(+)-1-(tetrahydrofuran-2-yl)methanamine (117 mg, 1.16 mmol, CAS No: 7175-81-7) as starting material.

Specific optical rotation (Method 11): +9.3 (c=1.0 g/100 ml in DMSO)

### Example 72

### N-{trans-4-[(2-chloro-4-fluorophenyl)carbamoyl]cyclohexyl}-4-[(3,3-difluoroazetidin-1-yl)carbonyl]-1H-imidazole-5-carboxamide

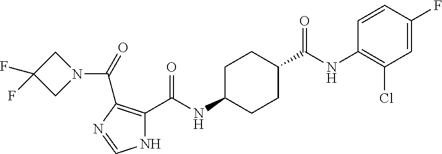 [SMILES: O=C(N[C@H]1CC[C@H](C(=O)Nc2ccc(F)cc2Cl)CC1)c1[nH]cnc1C(=O)N1CC(F)(F)C1]

A mixture of phenyl 5-({trans-4-[(2-chloro-4-fluorophenyl)carbamoyl]cyclohexyl}carbamoyl)-1H-imidazole-4-carboxylate (100 mg, 0.206 mmol), 3,3-difluoroazetidine hydrochloride (134 mg, 1.03 mmol, CAS No 288315-03-7) and triethylamine (144 μl, 1.03 mmol) in tetrahydrofuran (3.2 ml) was stirred at 60° C. over night. For work-up, the reaction mixture was concentrated and the residue was purified by recrystallization from methanol to yield the title compound (73.0 mg, 73% yield).

LC-MS (Method 6): Rₜ=1.08 min; MS (ESIpos) m/z=484.1 [M+H]⁺.

¹H-NMR (400 MHz, DMSO-d₆): δ [ppm]=13.41 (br. s, 1H), 10.86 (d, 1H), 9.50 (s, 1H), 7.86 (s, 1H), 7.65-7.44 (m, 2H), 7.26-7.15 (m, 1H), 5.01 (t, 2H), 4.53 (t, 2H), 3.81-3.65 (m, 1H), 2.17-1.81 (m, 4H), 1.67-1.44 (m, 2H), 1.42-1.20 (m, 2H).

### Example 73

### N⁵-{trans-4-[(2-chloro-4-fluorophenyl)carbamoyl]cyclohexyl}-N⁴-(2-methoxy-2-methylpropyl)-1H-imidazole-4,5-dicarboxamide

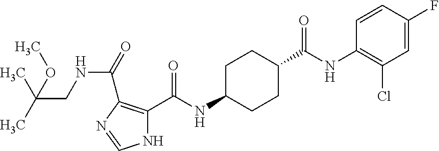 [SMILES: COC(C)(C)CNC(=O)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (150 mg, 0.309 mmol) and 2-methoxy-2-methylpropan-1-amine (160 mg, 1.55 mmol, CAS No 89282-70-2) in tetrahydrofuran (4.8 ml) was stirred at 60° C. over night. For work-up, the reaction mixture was concentrated and the residue was purified by flash chromatography (25 g Snap Cartridge, ethyl acetate/ethanol gradient, 0%->5% ethanol) to yield the title compound (18.0 mg, 12 yield).

LC-MS (Method 6): Rₜ=1.08 min; MS (ESIpos) m/z=494.2 [M+H]⁺.

¹H-NMR (400 MHz, DMSO-d₆): δ [ppm]=13.20-12.45 (m, 1H), 11.43-10.70 (m, 1H), 9.52 (s, 1H), 8.69-7.89 (m, 1H), 7.82 (s, 1H), 7.65-7.57 (m, 1H), 7.53-7.47 (m, 1H), 7.27-7.18 (m, 1H), 3.89-3.66 (m, 1H), 3.37 (d, 2H), 3.15 (s, 3H), 2.48-2.38 (m, 1H), 2.10-1.75 (m, 4H), 1.71-1.32 (m, 4H), 1.14 (s, 6H).

### Example 74

### N⁵-{trans-4-[(2-chloro-4-fluorophenyl)carbamoyl]cyclohexyl}-N⁴-(2-cyclopropylethyl)-1H-imidazole-4,5-dicarboxamide

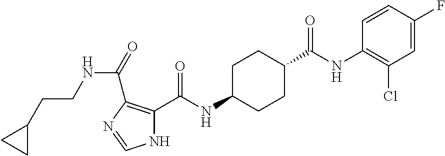 [SMILES: O=C(NCCC1CC1)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (100 mg, 0.206 mmol) and 2-cyclopropylethanamine (88 mg, 1.03 mmol, CAS No 62893-54-3) in tetrahydrofuran (3.2 ml) was stirred at 60° C. over night. For work-up the reaction mixture was concentrated and the residue was recrystalized to yield the title compound (57.0 mg, 58% yield).

LC-MS (Method 6): Rₜ=1.18 min; MS (ESIpos) m/z=476.2 [M+H]⁺.

¹H-NMR (400 MHz, DMSO-d₆): δ [ppm]=13.23-12.80 (m, 1H), 11.55-10.78 (m, 1H), 9.51 (s, 1H), 8.92-8.12 (m, 1H), 7.81 (s, 1H), 7.61 (dd, 1H), 7.50 (dd, 1H), 7.22 (td, 1H), 3.86-3.62 (m, 1H), 3.42-3.35 (m, 2H), 2.48-2.39 (m, 1H), 2.01-1.84 (m, 4H), 1.65-1.19 (m, 6H), 0.79-0.60 (m, 1H), 0.47-0.37 (m, 2H), 0.12-0.03 (m, 2H).

### Example 75

### N⁵-{trans-4-[(2-chloro-4-fluorophenyl)carbamoyl]cyclohexyl}-N⁴-(2-hydroxy-2-methylpropyl)-1H-imidazole-4,5-dicarboxamide

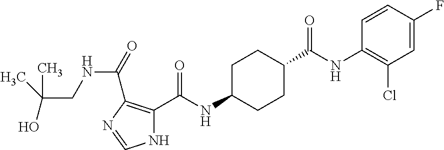 [SMILES: CC(C)(O)CNC(=O)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (150 mg, 0.309 mmol) and 1-amino-2-methylpropan-2-ol (138 mg, 1.55 mmol, CAS No 2854-16-2) in tetrahydrofuran (4.8 ml) was stirred at 60° C. over night. For work-up, the reaction mixture was concentrated and the residue was purified by preparative HPLC (Method 9) to yield the title compound (42.0 mg, 28% yield).

LC-MS (Method 3): Rₜ=0.94 min; MS (ESIpos) m/z=480.2 [M+H]⁺.

¹H-NMR (400 MHz, DMSO-d₆): δ [ppm]=13.35-13.12 (m, 1H), 11.19-11.04 (m, 1H), 9.56-9.45 (m, 1H), 8.42-8.32 (m, 0.5H), 8.24-8.17 (m, 0.5H), 7.82 (d, 1H), 7.66-7.56 (m, 1H), 7.55-7.45 (m, 1H), 7.26-7.16 (m, 1H), 4.76 (s, 0.5), 4.54 (s, 0.5H), 3.89-3.65 (m, 1H), 3.27 (d, 2H), 2.11-1.78 (m, 4H), 1.64-1.44 (m, 3H), 1.38-1.20 (m, 1H), 1.13 (s, 6H).

### Example 76

### N⁵-{trans-4-[(2-chloro-4-fluorophenyl)carbamoyl]cyclohexyl}-N⁴-(2-isopropoxyethyl)-1H-imidazole-4,5-dicarboxamide

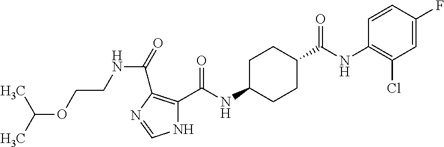 [SMILES: CC(C)OCCNC(=O)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (100 mg, 0.206 mmol) and 2-(propan-2-yloxy)ethanamine (106 mg, 1.03 mmol, CAS No 81731-43-3) in tetrahydrofuran (3.2 ml) was stirred at 60° C. over night. For work-up, the reaction mixture was concentrated and the residue was recrystalized from methanol to yield the title compound (70.0 mg, 69% yield).

LC-MS (Method 7): Rₜ=1.08 min; MS (ESIpos) m/z=494.2 [M+H]⁺.

¹H-NMR (400 MHz, DMSO-d₆): δ [ppm]=13.51-13.00 (m, 1H), 11.42-10.85 (m, 1H), 9.51 (s, 1H), 8.68-8.14 (m, 1H), 7.82 (s, 1H), 7.61 (dd, 1H), 7.50 (dd, 1H), 7.22 (td, 1H), 3.82-3.66 (m, 1H), 3.59 (spt, 1H), 3.52-3.46 (m, 2H), 3.46-3.39 (m, 2H), 2.48-2.39 (m, 1H), 2.15-1.80 (m, 4H), 1.66-1.21 (m, 4H), 1.10 (d, 6H).

### Example 77

### N⁵-{trans-4-[(2-chloro-4-fluorophenyl)carbamoyl]cyclohexyl}-N⁴-[2-(isopropylamino)ethyl]-1H-imidazole-4,5-dicarboxamide

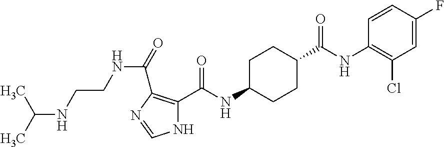 [SMILES: CC(C)NCCNC(=O)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (100 mg, 0.206 mmol) and N-(propan-2-yl)ethane-1,2-diamine (105 mg, 1.03 mmol, CAS No 19522-67-9) in tetrahydrofuran (3.2 ml) was stirred at 60° C. over night. For work-up, the reaction mixture was concentrated and the residue was recrystallized from methanol to yield the title compound (70.0 mg, 69% yield).

LC-MS (Method 7): Rₜ=0.71 min; MS (ESIpos) m/z=493.2 [M+H]⁺.

¹H-NMR (400 MHz, DMSO-d₆): δ [ppm]=11.39-11.22 (m, 0.4H), 11.15-10.99 (m, 0.6H), 9.59-9.45 (m, 1H), 8.84-8.66 (m, 0.6H), 8.58-8.38 (m, 0.4H), 7.85 (s, 1H), 7.60 (dd, 1H), 7.50 (dd, 1H), 7.22 (td, 1H), 3.87-3.63 (m, 1H), 3.57-3.43 (m, 2H), 3.18-2.99 (m, 1H), 2.99-2.83 (m, 2H), 2.48-2.38 (m, 1H), 2.16-1.78 (m, 4H), 1.66-1.20 (m, 4H), 1.12 (d, 6H).

### Example 78

### N⁵-{trans-4-[(2-chloro-4-fluorophenyl)carbamoyl]cyclohexyl}-N⁴-(tetrahydrofuran-2-ylmethyl)-1H-imidazole-4,5-dicarboxamide

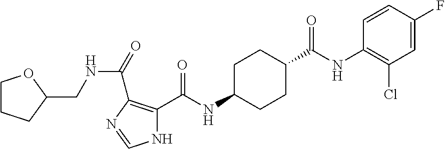 [SMILES: O=C(NCC1CCCO1)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (100 mg, 93% purity, 0.192 mmol) and 1-(tetrahydrofuran-2-yl)methanamine (97 mg, 0.96 mmol, CAS No 4795-29-3) in tetrahydrofuran (3.0 ml) was stirred room temperature over night. For work-up, the reaction mixture was concentrated and the residue was recrystallized from methanol to yield the title compound (53.0 mg, 55% yield).

LC-MS (Method 6): Rₜ=1.03 min; MS (ESIpos) m/z=492.2 [M+H]⁺.

¹H-NMR (400 MHz, DMSO-d₆): δ [ppm]=12.84-11.95 (m, 1H), 11.56-10.67 (m, 1H), 9.50 (s, 1H), 8.80-8.17 (m, 1H), 7.82 (s, 1H), 7.61 (dd, 1H), 7.50 (dd, 1H), 7.22 (td, 1H), 4.04-3.91 (m, 1H), 3.85-3.68 (m, 2H), 3.68-3.58 (m, 1H), 3.44-3.34 (m, 2H), 2.17-1.69 (m, 7H), 1.68-1.20 (m, 5H).

### Example 79

### N⁵-{trans-4-[(2-chloro-4-fluorophenyl)carbamoyl]cyclohexyl}-N⁴-[(2S)-tetrahydrofuran-2-ylmethyl]-1H-imidazole-4,5-dicarboxamide

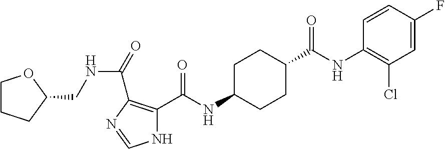 [SMILES: O=C(NC[C@@H]1CCCO1)c1nc[nH]c1C(=O)N[C@H]1CC[C@H](C(=O)Nc2ccc(F)cc2Cl)CC1]

Was prepared in analogy to the synthesis of N⁵-{trans-4-[(2-chloro-4-fluorophenyl)carbamoyl]cyclohexyl}-N⁴-(tetrahydrofuran-2-ylmethyl)-1H-imidazole-4,5-dicarboxamide was prepared using (S)-(+)-1-(tetrahydrofuran-2-yl)methanamine (104 mg, 1.03 mmol, CAS No: 7175-81-7 as starting material.

Specific optical rotation (Method 11): +7.7 (c=1.0 g/100 ml in DMSO)

### Example 80

### N⁵-{trans-4-[(2-chloro-4-fluorophenyl)carbamoyl]cyclohexyl}-N⁴-[(2R)-tetrahydrofuran-2-ylmethyl]-1H-imidazole-4,5-dicarboxamide

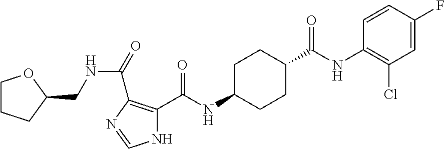 [SMILES: O=C(NC[C@H]1CCCO1)c1nc[nH]c1C(=O)N[C@H]1CC[C@H](C(=O)Nc2ccc(F)cc2Cl)CC1]

Was prepared in analogy to the synthesis of N⁵-{trans-4-[(2-chloro-4-fluorophenyl)carbamoyl]cyclohexyl}-N⁴-(tetrahydrofuran-2-ylmethyl)-1H-imidazole-4,5-dicarboxamide was prepared using (R)-(−)-1-(tetrahydrofuran-2-yl)methanamine (104 mg, 1.03 mmol, Cas No: 7202-43-9) as starting material.

Specific optical rotation (Method 11): −9.6 (c=1.0 g/100 ml in DMSO)

### Example 81

### N⁵-{trans-4-[(2-chloro-4-fluorophenyl)carbamoyl]cyclohexyl}-N⁴-(2-methoxypropyl)-1H-imidazole-4,5-dicarboxamide

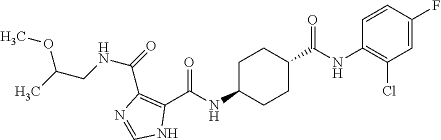 [SMILES: COC(C)CNC(=O)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (100 mg, 0.206 mmol), 2-methoxypropan-1-amine hydrochloride (126 mg, 1.03 mmol, CAS No 70807-90-8) and triethylamine (144 μl, 1.03 mmol) in tetrahydrofuran (3.2 ml) was stirred at room temperature over night. For work-up, the reaction mixture was concentrated and the residue was purified by flash chromatography (25 g Snap-Catridge, ethyl acetate/ethanol gradient 0%->5% ethanol) to yield the title compound (70.0 g, 67% yield).

LC-MS (Method 6): Rₜ=1.03 min; MS (ESIpos) m/z=480.2 [M+H]⁺.

¹H-NMR (400 MHz, DMSO-d₆): δ [ppm]=13.32-13.13 (m, 1H), 11.27-11.02 (m, 1H), 9.60-9.42 (m, 1H), 8.47-8.28 (m, 1H), 7.82 (s, 1H), 7.66-7.55 (m, 1H), 7.54-7.42 (m, 1H), 7.27-7.14 (m, 1H), 3.99-3.64 (m, 1H), 3.58-3.35 (m, 2H), 3.28 (s, 3H), 2.10-1.1.18 (m, 8H), 1.17-1.00 (m, 3H).

### Example 82

### N⁵-{trans-4-[(2-chloro-4-fluorophenyl)carbamoyl]cyclohexyl}-N⁴-(1-methoxypropan-2-yl)-1H-imidazole-4,5-dicarboxamide

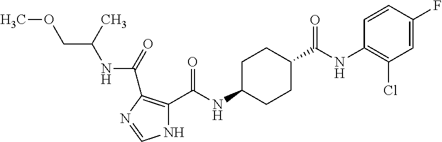 [SMILES: COCC(C)NC(=O)c1nc[nH]c1C(=O)N[C@H]1CC[C@H](C(=O)Nc2ccc(F)cc2Cl)CC1]

A mixture of phenyl 5-({trans-4-[(2-chloro-4-fluorophenyl)carbamoyl]cyclohexyl}carbamoyl)-1H-imidazole-4-carboxylate (100 mg, 93% purity, 0.192 mmol) and 1-methoxypropan-2-amine (85 mg, 0.96 mmol, CAS No 37143-54-7) in tetrahydrofuran (3.0 ml) was stirred at room temperature over night. For work-up, the reaction mixture filtrated and the filtrate was purified by purified by preparative HPLC (Method 9) to yield the title compound (15.0 mg, 16% yield).

LC-MS (Method 3): Rₜ=1.05 min; MS (ESIpos) m/z=480.2 [M+H]⁺.

¹H-NMR (400 MHz, DMSO-d₆): δ [ppm]=13.31-13.13 (m, 1H), 11.29-11.00 (m, 1H), 9.51 (s, 1H), 8.56-8.14 (m, 1H), 7.82 (s, 1H), 7.65-7.55 (m, 1H), 7.53-7.46 (m, 1H), 7.28-7.16 (m, 1H), 4.33-4.05 (m, 1H), 3.93-3.60 (m, 1H), 3.49-3.38 (m, 1H), 3.28 (s, 3H), 2.47-2.39 (m, 1H), 2.16-1.78 (m, 4H), 1.70-1.22 (m, 4H), 1.16 (d, 3H).

### Example 83

### N⁵-{trans-4-[(2-chloro-4-fluorophenyl)carbamoyl]cyclohexyl}-N⁴-[(2S)-1-methoxypropan-2-yl]-1H-imidazole-4,5-dicarboxamide

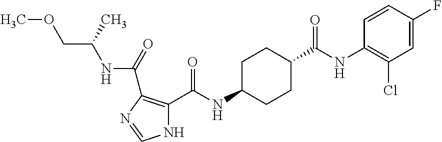 [SMILES: COC[C@H](C)NC(=O)c1nc[nH]c1C(=O)N[C@H]1CC[C@H](C(=O)Nc2ccc(F)cc2Cl)CC1]

Was prepared in analogy to the synthesis of N⁵-{trans-4-[(2-chloro-4-fluorophenyl)carbamoyl]cyclohexyl}-N⁴-(1-methoxypropan-2-yl)-1H-imidazole-4,5-dicarboxamide using (2S)-(+)-1-methoxypropan-2-amine (92 mg, 1.03 mmol, CAS No: 99636-32-5) as starting material.

Specific optical rotation (Method 11): +1.8 (c=1.0 g/100 ml in DMSO)

### Example 84

### N⁵-{trans-4-[(2-chloro-4-fluorophenyl)carbamoyl]cyclohexyl}-N⁴-[(2R)-1-methoxypropan-2-yl]-1H-imidazole-4,5-dicarboxamide

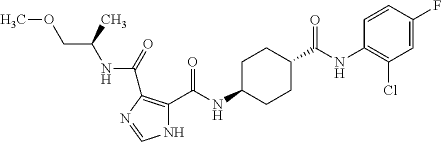 [SMILES: COC[C@@H](C)NC(=O)c1nc[nH]c1C(=O)N[C@H]1CC[C@H](C(=O)Nc2ccc(F)cc2Cl)CC1]

Was prepared in analogy to the synthesis of N⁵-{trans-4-[(2-chloro-4-fluorophenyl)carbamoyl]cyclohexyl}-N⁴-(1-methoxypropan-2-yl)-1H-imidazole-4,5-dicarboxamide using (2R)-(−)1-methoxypropan-2-amine hydrochloride (130 mg, 1.03 mmol) as starting material and triethylamine (144 μl, 1.03 mmol) as additional base.

Specific optical rotation (Method 11): −3.4 (c=1.0 g/100 ml in DMSO)

### Example 85

### N⁵-{trans-4-[(2-chloro-4,5-difluorophenyl)carbamoyl]cyclohexyl}-N⁴-(2-methoxy-2-methylpropyl)-1H-imidazole-4,5-dicarboxamide

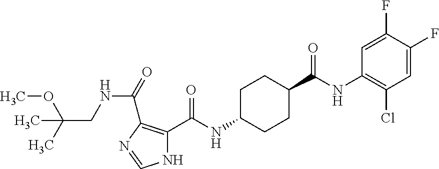 [SMILES: COC(C)(C)CNC(=O)c1nc[nH]c1C(=O)N[C@H]1CC[C@H](C(=O)Nc2cc(F)c(F)cc2Cl)CC1]

A mixture of phenyl 5-({trans-4-[(2-chloro-4,5-difluorophenyl)carbamoyl]cyclohexyl}carbamoyl)-1H-imidazole-4-carboxylate (100 mg, 0.199 mmol) and 2-methoxy-2-methylpropan-1-amine (103 mg, 0.994 mmol, CAS No 89282-70-2) in tetrahydrofuran (5.0 ml) was stirred at 60° C. over night. For work-up, the reaction mixture was concentrated and the residue was first purified by flash chromatography (10 g Snap Cartridge, ethyl acetate/ethanol gradient, 0%->5% ethanol) followed by purification with preparative HPLC (Method 9) to yield the title compound (46.0 mg, 45% yield).

LC-MS (Method 3): Rₜ=1.15 min; MS (ESIpos) m/z=512.2 [M+H]⁺.

¹H-NMR (400 MHz, DMSO-d₆): δ [ppm]=13.38-13.09 (m, 1H), 11.25-10.90 (m, 1H), 9.67-9.53 (m, 1H), 8.52-8.25 (m, 0.5H), 8.21-7.95 (m, 0.5H), 7.87-7.75 (m, 3H), 3.92-3.59 (m, 1H), 3.37 (d, 2H), 3.15 (s, 3H), 2.15-1.77 (m, 4H), 1.65-1.20 (m, 4H), 1.14 (s, 6H).

### Example 86

### N⁵-{trans-4-[(2-chloro-4,5-difluorophenyl)carbamoyl]cyclohexyl}-N⁴-(tetrahydrofuran-2-ylmethyl)-1H-imidazole-4,5-dicarboxamide

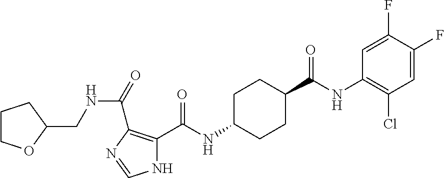 [SMILES: O=C(NCC1CCCO1)c1nc[nH]c1C(=O)N[C@H]1CC[C@H](C(=O)Nc2cc(F)c(F)cc2Cl)CC1]

A mixture of phenyl 5-({trans-4-[(2-chloro-4,5-difluorophenyl)carbamoyl]cyclohexyl}carbamoyl)-1H-imidazole-4-carboxylate (100 mg, 0.199 mmol) and 1-(tetrahydrofuran-2-yl)methanamine (101 mg, 0.994 mmol, CAS No 4795-29-3) in tetrahydrofuran (5.0 ml) was stirred at 60° C. over night. For work-up the reaction mixture was concentrated and the residue was purified recrystaliztaion from methanol to yield the title compound (35.0 mg, 35% yield).

LC-MS (Method 6): Rₜ=1.11 min; MS (ESIpos) m/z=510.2 [M+H]⁺.

¹H-NMR (400 MHz, DMSO-d₆): δ [ppm]=13.04-12.49 (m, 1H), 11.47-10.77 (m, 1H), 9.56 (s, 1H), 8.71-8.09 (m, 1H), 7.90-7.67 (m, 3H), 4.03-3.87 (m, 1H), 3.82-3.67 (m, 2H), 3.65-3.53 (m, 1H), 3.42-3.32 (m, 2H), 2.13-1.70 (m, 7H), 1.64-1.16 (m, 6H).

### Example 87

### N⁵-{trans-4-[(2-chloro-4,5-difluorophenyl)carbamoyl]cyclohexyl}-N⁴-(1-methoxypropan-2-yl)-1H-imidazole-4,5-dicarboxamide

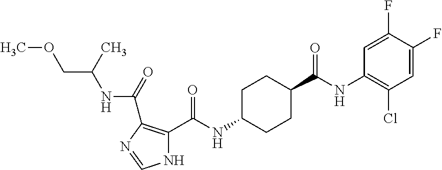 [SMILES: COCC(C)NC(=O)c1nc[nH]c1C(=O)N[C@H]1CC[C@H](C(=O)Nc2cc(F)c(F)cc2Cl)CC1]

A mixture of phenyl 5-({trans-4-[(2-chloro-4,5-difluorophenyl)carbamoyl]cyclohexyl}carbamoyl)-1H-imidazole-4-carboxylate (100 mg, 0.199 mmol) and 1-methoxypropan-2-amine (89 mg, 0.994 mmol, CAS No 37143-54-7) in tetrahydrofuran (5.0 ml) was stirred at 600° C. over night. For work-up the reaction mixture was concentrated and the residue was purified with preparative HPLC (Method 9) to yield the title compound (49.5 mg, 50% yield).

LC-MS (Method 3): Rₜ=1.11 min; MS (ESIpos) m/z=498.2 [M+H]⁺.

¹H-NMR (400 MHz, DMSO-d₆): δ [ppm]=13.19 (br. s., 1H), 9.61 (s, 1H), 7.87-7.74 (m, 3H), 4.23-4.11 (m, 1H), 3.84-3.65 (m, 1H), 3.47-3.39 (m, 1H), 3.28 (s, 3H), 2.14-1.81 (m, 4H), 1.65-1.28 (m, 4H), 1.16 (d, 3H).

### Example 88

### N⁵-(trans-4-{[2-chloro-5-(morpholin-4-yl)phenyl]carbamoyl}cyclohexyl)-N⁴-methyl-1H-imidazole-4,5-dicarboxamide

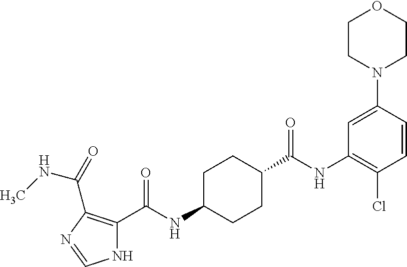 [SMILES: CNC(=O)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (120 mg, 0.217 mmol) and methanamine (0.54 ml, 1.1 mmol, 2 M solution in tetrahydrofuran) in tetrahydrofuran (5.5 ml) was stirred at 60° C. over night. The precipitate was collected by filtration washed with methanol and dried to yield the title compound (63.0 mg, 59% yield).

LC-MS (Method 6): Rₜ=0.93 min; MS (ESIpos) m/z=489.2 [M+H]⁺.

¹H-NMR (400 MHz, DMSO-d₆): δ [ppm]=10.12-9.34 (m, 2H), 9.31 (s, 1H), 7.34-7.24 (m, 2H), 7.23-7.12 (m, 1H), 6.82-6.75 (m, 1H), 3.84-3.57 (m, 5H), 3.13-3.02 (m, 4H), 2.77 (d, 3H), 2.05-1.83 (m, 4H), 1.59-1.43 (m, 2H), 1.42-1.28 (m, 2H).

### Example 89

### N⁵-(trans-4-{[2-chloro-5-(morpholin-4-yl)phenyl]carbamoyl}cyclohexyl)-N⁴-(tetrahydrofuran-2-ylmethyl)-1H-imidazole-4,5-dicarboxamide

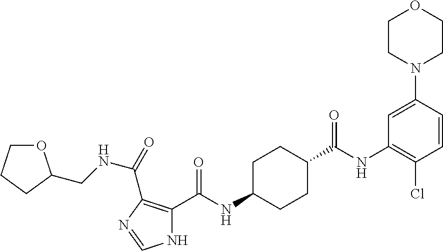 [SMILES: O=C(NCC1CCCO1)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol) and 1-(tetrahydrofuran-2-yl)methanamine (92 mg, 0.906 mmol) in tetrahydrofuran (4.6 ml) was stirred at 60° C. over night. For work-u p the reaction mixture was concentrated and purified by preparative HPLC (Method 9) to yield the title compound (10.0 mg, 10% yield).

LC-MS (Method 3): Rₜ=1.01 min; MS (ESIpos) m/z=559.3 [M+H]⁺.

¹H-NMR (400 MHz, DMSO-d₆): δ [ppm]=13.39-13.09 (m, 1H), 11.52-10.71 (m, 1H), 9.36-9.28 (m, 1H), 8.66-8.15 (m, 1H), 7.82 (s, 1H), 7.32-7.26 (m, 2H), 6.81-6.76 (m, 1H), 4.04-3.91 (m, 1H), 3.85-3.60 (m, 7H), 3.14-2.99 (m, 4H), 2.10-1.72 (m, 7H), 1.65-1.22 (m, 5H).

### Example 90

### N⁵-(trans-4-{[2-chloro-5-(morpholin-4-yl)phenyl]carbamoyl}cyclohexyl)-N⁴-(2-methoxy-2-methylpropyl)-1H-imidazole-4,5-dicarboxamide

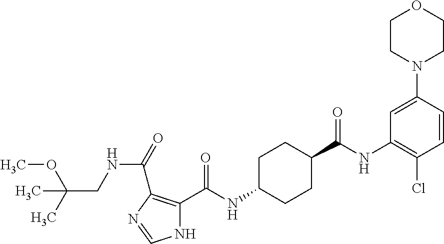 [SMILES: COC(C)(C)CNC(=O)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol) and 2-methoxy-2-methylpropan-1-amine (93 mg, 0.906 mmol, CAS No 89282-70-2) in tetrahydrofuran (4.6 ml) was stirred at 60° C. over night. For work-up the reaction mixture was concentrated and purified by preparative HPLC (Method 9) to yield the title compound (5.2 mg, 5% yield).

LC-MS (Method 6): Rₜ=1.08 min; MS (ESIpos) m/z=561.3 [M+H]⁺.

¹H-NMR (400 MHz, DMSO-d₆): δ [ppm]=10.07-9.57 (m, 1H), 9.51-9.04 (m, 2H), 7.35-7.11 (m, 3H), 6.81-6.76 (m, 1H), 3.82-3.61 (m, 5H), 3.15 (s, 3H), 3.13-3.03 (m, 4H), 2.02-1.86 (m, 4H), 1.62-1.43 (m, 2H), 1.42-1.27 (m, 2H), 1.13 (s, 6H).

### Example 91

### N⁵-(trans-4-{[2-chloro-5-(morpholin-4-yl)phenyl]carbamoyl}cyclohexyl)-N⁴-(1-methoxypropan-2-yl)-1H-imidazole-4,5-dicarboxamide

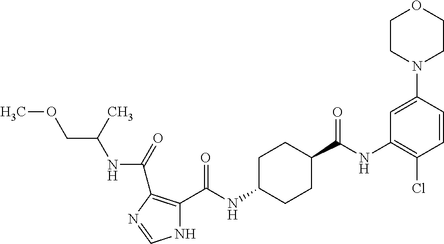 [SMILES: COCC(C)NC(=O)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol) 1-methoxypropan-2-amine (81 mg, 0.906 mmol, CAS No 37143-54-7) in tetrahydrofuran (4.6 ml) was stirred at 60° C. over night. For work-up, the precipitate was filtrated off, washed with methanol and the eluent was concentrated and purified by preparative HPLC (Method 9) to yield the title compound (11.0 mg, 11% yield).

LC-MS (Method 3): Rₜ=1.03 min; MS (ESIpos) m/z=547.3 [M+H]⁺.

¹H-NMR (400 MHz, DMSO-d₆): δ [ppm]=13.34-12.96 (m, 1H), 9.31 (s, 1H), 7.74 (s, 1H), 7.35-7.21 (m, 2H), 6.82-6.72 (m, 1H), 4.26-4.09 (m, 1H), 3.81-3.67 (m, 5H), 3.46-3.39 (m, 1H), 3.28 (s, 3H), 3.11-3.00 (m, 4H), 2.06-1.84 (m, 4H), 1.65-1.31 (m, 4H), 1.16 (d, 3H).

### Example 92

### N-(trans-4-{[2-chloro-5-(morpholin-4-yl)phenyl]carbamoyl}cyclohexyl)-4-[(3,3-difluoroazetidin-1-yl)carbonyl]-1H-imidazole-5-carboxamide

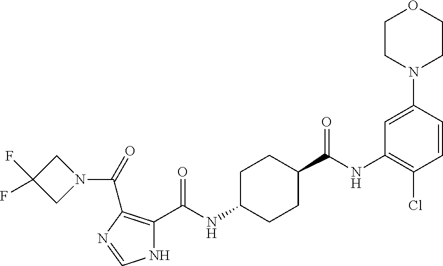 [SMILES: O=C(N[C@H]1CC[C@H](C(=O)Nc2cc(N3CCOCC3)ccc2Cl)CC1)c1[nH]cnc1C(=O)N1CC(F)(F)C1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol), 3,3-difluoroazetidine hydrochloride (117 mg, 0.906 mmol, CAS No 288315-03-7) and triethylamine (126 μl, 0.906 mmol) in tetrahydrofuran (4.5 ml) was stirred at 60° C. over night. For work-up, the reaction mixture was concentrated and the residue was purified by recrystallization from methanol to yield the title compound (50.0 mg, 50% yield).

LC-MS (Method 6): Rₜ=1.09 min; MS (ESIpos) m/z=551.2 [M+H]⁺.

¹H-NMR (400 MHz, DMSO-d₆): δ [ppm]=11.27-11.00 (m, 1H), 9.35 (s, 1H), 7.32-7.20 (m, 3H), 6.83-6.73 (m, 1H), 4.98-4.84 (m, 2H), 4.51-4.34 (m, 2H), 3.78-3.66 (m, 5H), 3.12-3.00 (m, 4H), 2.11-1.87 (m, 4H), 1.65-1.40 (m, 2H), 1.40-1.21 (m, 2H).

### Example 93

### N⁵-(trans-4-{[2-chloro-5-(morpholin-4-yl)phenyl]carbamoyl}cyclohexyl)-N⁴-(2-cyclopropylethyl)-1H-imidazole-4,5-dicarboxamide

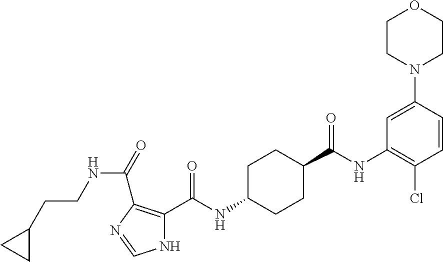 [SMILES: O=C(NCCC1CC1)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol) and 2-cyclopropylethanamine (77 mg, 0.906 mmol, CAS No 62893-54-3) in tetrahydrofuran (4.6 ml) was stirred at 60° C. over nigh t. For work-up, the reaction mixture was concentrated and the residue purified by preparative HPLC (Method 9) to yield the title compound (14.0 mg, 14% yield).

LC-MS (Method 3): Rₜ=1.17 min; MS (ESIpos) m/z=543.3 [M+H]⁺.

¹H-NMR (400 MHz, DMSO-d₆): δ [ppm]=13.27-13.14 (m, 1H), 11.26-11.12 (m, 1H), 9.36-9.25 (m, 1H), 8.64-8.34 (m, 1H), 7.81 (s, 1H), 7.34-7.25 (m, 2H), 6.83-6.74 (m, 1H), 3.86-3.62 (m, 5H), 3.12-3.03 (m, 4H), 2.10-1.79 (m, 5H), 1.63-1.19 (m, 7H), 0.91-0.61 (m, 1H), 0.46-0.37 (m, 2H), 0.13-0.02 (m, 2H).

### Example 94

### N⁵-(trans-4-{[2-chloro-5-(morpholin-4-yl)phenyl]carbamoyl}cyclohexyl)-N⁴-(2-hydroxy-2-methylpropyl)-1H-imidazole-4,5-dicarboxamide

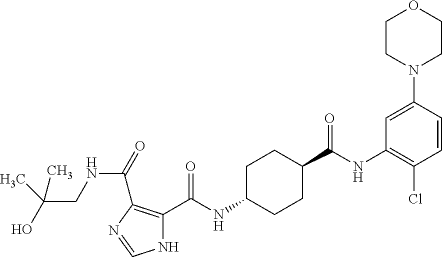 [SMILES: CC(C)(O)CNC(=O)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol) and 1-amino-2-methylpropan-2-ol (81 mg, 0.906 mmol, CAS No 2854-16-2) in tetrahydrofuran (4.6 ml) was stirred at 60° C. over nigh t. For work-up, the reaction mixture was concentrated and the residue was purified by preparative HPLC (Method 9) to yield the title compound (12.0 mg, 12% yield).

LC-MS (Method 3): Rₜ=0.93 min; MS (ESIpos) m/z=547.3 [M+H]⁺.

¹H-NMR (400 MHz, DMSO-d₆): δ [ppm]=13.35-12.98 (m, 1H), 11.45-10.73 (m, 1H), 9.31 (s, 1H), 8.75-7.88 (m, 1H), 7.81 (s, 1H), 7.34-7.20 (m, 2H), 6.85-6.73 (m, 1H), 4.86-4.41 (m, 1H), 3.83-3.66 (m, 5H), 3.27 (d, 2H), 3.14-2.99 (m, 4H), 2.15-1.65 (m, 4H), 1.65-1.19 (m, 4H), 1.13 (s, 6H).

### Example 95

### N⁵-(trans-4-{[2-chloro-5-(morpholin-4-yl)phenyl]carbamoyl}cyclohexyl)-N⁴-(2,2,2-trifluoroethyl)-1H-imidazole-4,5-dicarboxamide

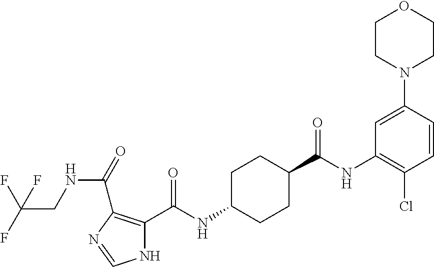 [SMILES: O=C(NCC(F)(F)F)c1nc[nH]c1C(=O)N[C@H]1CC[C@H](C(=O)Nc2cc(N3CCOCC3)ccc2Cl)CC1]

A mixture of phenyl 5-[(trans-4-{[2-chloro-5-(morpholin-4-yl)phenyl]carbamoyl}cyclohexyl)carbamoyl]-1H-imidazole-4-carboxylate (100 mg, 0.181 mmol), 2,2,2-trifluoroethanamine (90 mg, 0.906 mmol, CAS No 753-90-2) and triethylamine (126 μl, 0.906 mmol) in tetrahydrofuran (4.6 ml) was stirred at room temperature over night. For work-up, the reaction mixture was concentrated and the residue was purified by preparative HPLC (Method 9) to yield the title compound (2.9 mg, 3% yield).

LC-MS (Method 3): Rₜ=1.03 min; MS (ESIpos) m/z=557.2 [M+H]⁺.

¹H-NMR (400 MHz, DMSO-d₆): δ [ppm]=13.59-13.25 (m, 1H), 11.93-11.75 (m, 0.5H), 10.83-10.65 (m, 0.5H), 9.39-9.06 (m, 1.5H), 8.67-8.44 (m, 0.5H), 7.97-7.81 (m, 1H), 7.35-7.17 (m, 2H), 6.84-6.74 (m, 1H), 4.31-3.98 (m, 2H), 3.91-3.62 (m, 5H), 3.13-2.99 (m, 4H), 2.14-1.78 (m, 4H), 1.66-1.18 (m, 4H).

### Example 96

### N⁵-{trans-4-[(4-chloropyridin-3-yl)carbamoyl]cyclohexyl}-N⁴-methyl-1H-imidazole-4,5-dicarboxamide

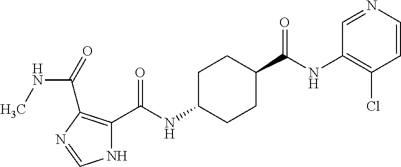 [SMILES: CNC(=O)c1nc[nH]c1C(=O)N[C@H]1CC[C@H](C(=O)Nc2cnccc2Cl)CC1]

A mixture of phenyl 5-({trans-4-[(4-chloropyridin-3-yl)carbamoyl]cyclohexyl}carbamoyl)-1H-imidazole-4-carboxylate (105 mg, 0.224 mmol) and methanamine (0.56 ml, 1.1 mmol, 2 M solution in THF) in tetrahydrofuran (5.6 ml) was stirred at 60° C. over night. For work-up the precipitate was collected by filtration, washed with methanol and dried to yield the title compound (32.0 mg, 35% yield).

LC-MS (Method 6): Rₜ=0.66 min; MS (ESIpos) m/z=405.1 [M+H]⁺.

¹H-NMR (400 MHz, DMSO-d₆): δ [ppm]=13.19 (br. s., 1H), 11.38-10.84 (m, 1H), 9.75 (s, 1H), 8.79-8.28 (m, 3H), 7.81 (s, 1H), 7.62 (d, 1H), 3.92-3.65 (m, 1H), 2.82 (d, 3H), 2.21-1.80 (m, 4H), 1.69-1.14 (m, 4H).

### Example 97

### N⁵-{trans-4-[(4-chloropyridin-3-yl)carbamoyl]cyclohexyl}-N⁴-(tetrahydrofuran-2-ylmethyl)-1H-imidazole-4,5-dicarboxamide

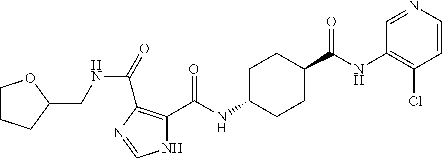 [SMILES: O=C(NCC1CCCO1)c1nc[nH]c1C(=O)N[C@H]1CC[C@H](C(=O)Nc2cnccc2Cl)CC1]

A mixture of phenyl 5-({trans-4-[(4-chloropyridin-3-yl)carbamoyl]cyclohexyl}carbamoyl)-1H-imidazole-4-carboxylate (95.0 mg, 0.203 mmol) and 1-(tetrahydrofuran-2-yl)methanamine (103 mg, 1.02 mmol) in tetrahydrofuran (5.1 ml) was stirred at 60° C. over night. For work-up, the reaction mixture was concentrated and the residue was purified by preparative HPLC (Method 9) to yield the title compound (35.0 mg, 36% yield).

LC-MS (Method 3): Rₜ=0.81 min; MS (ESIpos) m/z=475.3 [M+H]⁺.

¹H-NMR (400 MHz, DMSO-d₆): δ [ppm]=13.31-12.89 (m, 1H), 11.45-10.69 (m, 1H), 9.76 (s, 1H), 8.74 (s, 1H), 8.60-8.29 (m, 2H), 7.82 (s, 1H), 7.62 (d, 1H), 4.04-3.91 (m, 1H), 3.86-3.69 (m, 2H), 3.69-3.59 (m, 1H), 3.43-3.35 (m, 2H), 2.14-1.70 (m, 7H), 1.68-1.13 (m, 5H).

### Example 98

### N⁵-{trans-4-[(4-chloropyridin-3-yl)carbamoyl]cyclohexyl}-N⁴-(2-methoxy-2-methylpropyl)-1H-imidazole-4,5-dicarboxamide

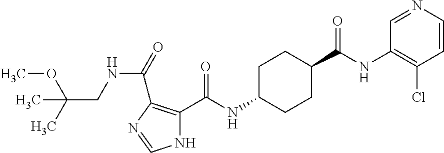 [SMILES: COC(C)(C)CNC(=O)c1nc[nH]c1C(=O)N[C@H]1CC[C@H](C(=O)Nc2cnccc2Cl)CC1]

A mixture of phenyl 5-({trans-4-[(4-chloropyridin-3-yl)carbamoyl]cyclohexyl}carbamoyl)-1H-imidazole-4-carboxylate (95.0 mg, 0.203 mmol) and 2-methoxy-2-methylpropan-1-amine (105 mg, 1.02 mmol, CAS No 89282-70-2) in tetrahydrofuran (5.2 ml) was stirred at 60° C. over night. For work-up, the reaction mixture was concentrated and the residue was purified by preparative HPLC (Method 9) to yield the title compound (32.0 mg, 33% yield).

LC-MS (Method 6): Rₜ=0.83 min; MS (ESIpos) m/z=477.2 [M+H]⁺.

¹H-NMR (400 MHz, DMSO-d₆): δ [ppm]=13.24 (br. s, 1H), 11.24-10.95 (m, 1H), 9.76 (s, 1H), 8.74 (s, 1H), 8.47-8.29 (m, 1.5H), 8.15-7.90 (m, 0.5H), 7.83 (s, 1H), 7.62 (d, 1H), 3.90-3.62 (m, 1H), 3.37 (d, 2H), 3.15 (s, 3H), 2.19-1.78 (m, 4H), 1.75-1.20 (m, 4H), 1.14 (s, 6H).

### Example 99

### N⁵-{trans-4-[(4-chloropyridin-3-yl)carbamoyl]cyclohexyl}-N⁴-(1-methoxypropan-2-yl)-1H-imidazole-4,5-dicarboxamide

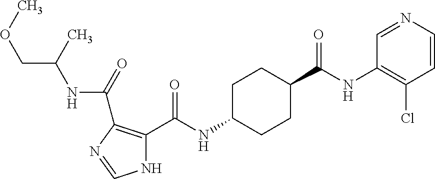 [SMILES: COCC(C)NC(=O)c1nc[nH]c1C(=O)N[C@H]1CC[C@H](C(=O)Nc2cnccc2Cl)CC1]

A mixture of phenyl 5-({trans-4-[(4-chloropyridin-3-yl)carbamoyl]cyclohexyl}carbamoyl)-1H-imidazole-4-carboxylate (90.0 mg, 0.192 mmol) and 1-methoxypropan-2-amine (86 mg, 0.862 mmol, CAS No 37143-54-7) in tetrahydrofuran (4.8 ml) was stirred at 60° C. over night. The precipitate collected by filtration, washed with methanol and dried to yield the title compound (25.0 mg, 27% yield).

LC-MS (Method 6): Rₜ=0.79 min; MS (ESIpos) m/z=463.2 [M+H]⁺.

¹H-NMR (400 MHz, DMSO-d₆): δ [ppm]=12.63-11.56 (m, 1H), 11.43-10.68 (m, 1H), 9.71 (s, 1H), 8.73 (s, 1H), 8.52-8.12 (m, 2H), 7.79 (s, 1H), 7.59 (d, 1H), 4.25-4.05 (m, 1H), 3.87-3.64 (m, 1H), 3.48-3.37 (m, 1H), 3.36-3.31 (m, 1H), 3.27 (s, 3H), 2.15-1.75 (m, 4H), 1.67-1.24 (m, 4H), 1.15 (d, 3H).

### Example 100

### N⁵-{trans-4-[(2-chloro-4-fluoro-5-methylphenyl)carbamoyl]cyclohexyl}-N⁴-(2-hydroxy-2-methylpropyl)-1H-imidazole-4,5-dicarboxamide

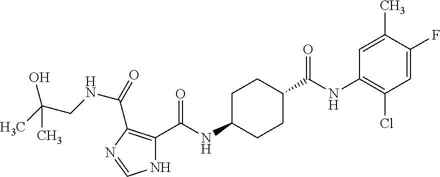 [SMILES: Cc1cc(NC(=O)[C@H]2CC[C@H](NC(=O)c3[nH]cnc3C(=O)NCC(C)(C)O)CC2)c(Cl)cc1F]

A mixture of phenyl 5-({trans-4-[(2-chloro-4-fluoro-5-methylphenyl)carbamoyl]cyclohexyl}carbamoyl)-1H-imidazole-4-carboxylate (100 mg, 0.200 mmol) and 1-amino-2-methylpropan-2-ol (89 mg, 1.02 mmol, CAS No 2854-16-2) in tetrahydrofuran (5.0 ml) was stirred at 60° C. over nigh t. For work-up, the reaction mixture was concentrated and the residue was purified by preparative HPLC (Method 9) to yield the title compound (48.0 mg, 48% yield).

LC-MS (Method 3): Rₜ=1.01 min; MS (ESIpos) m/z=494.3 [M+H]⁺.

¹H-NMR (400 MHz, DMSO-d₆): δ [ppm]=13.22 (br. s., 1H), 11.76-9.72 (m, 0.3H), 9.46 (s, 1H), 9.08-7.91 (m, 0.4H), 7.81 (s, 1H), 7.57-7.31 (m, 2H), 4.77-4.55 (m, 1H), 3.82-3.66 (m, 1H), 3.27 (d, 2H), 2.46-2.39 (m, 1H), 2.21 (d, 3H), 2.07-1.86 (m, 4H), 1.65-1.27 (m, 4H), 1.13 (s, 6H).

### Example 101

### N⁵-{trans-4-[(2-chloro-4-fluoro-5-methylphenyl)carbamoyl]cyclohexyl}-N⁴-(2-methoxy-2-methylpropyl)-1H-imidazole-4,5-dicarboxamide

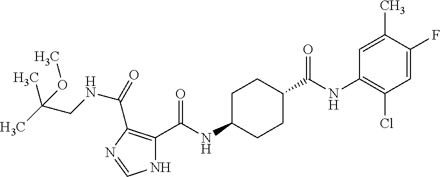 [SMILES: COC(C)(C)CNC(=O)c1nc[nH]c1C(=O)N[C@H]1CC[C@H](C(=O)Nc2cc(C)c(F)cc2Cl)CC1]

A mixture of phenyl 5-({trans-4-[(2-chloro-4-fluoro-5-methylphenyl)carbamoyl]cyclohexyl}carbamoyl)-1H-imidazole-4-carboxylate (100 mg, 0.200 mmol) and 2-methoxy-2-methylpropan-1-amine (103 mg, 1.00 mmol, CAS No 89282-70-2) in tetrahydrofuran (5.0 ml) was stirred at 60° C. over nigh t. For work-up, the reaction mixture was concentrated and the residue was purified by preparative HPLC (Method 9) to yield the title compound (48.0 mg, 48% yield).

LC-MS (Method 3): Rₜ=1.16 min; MS (ESIpos) m/z=508.3 [M+H]⁺.

¹H-NMR (400 MHz, DMSO-d₆): δ [ppm]=13.37-13.15 (m, 1H), 11.22-10.96 (m, 1H), 9.46 (br. s., 1H), 8.47-7.96 (m, 1H), 7.83 (s, 1H), 7.50 (d, 1H), 7.41 (d, 1H), 3.90-3.56 (m, 1H), 3.37 (d, 2H), 3.15 (br. s., 3H), 2.21 (d, 3H), 2.14-1.79 (m, 4H), 1.64-1.47 (m, 3H), 1.40-1.21 (m, 1H), 1.14 (s, 6H).

### Example 102

### N-{trans-4-[(3-chloropyridin-4-yl)carbamoyl]cyclohexyl}-5,7-dimethylpyrazolo[1,5-a]pyrimidine-3-carboxamide

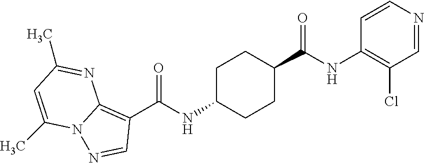 [SMILES: Cc1cc(C)n2ncc(C(=O)N[C@H]3CC[C@H](C(=O)Nc4ccncc4Cl)CC3)c2n1]

(Benzotriazol-1-yloxy)tripyrrolidinophosphonium hexafluorophosphate (261 mg, 0.502 mmol) was added to a mixture of 5,7-dimethyl-pyrazolo[1,5-a]pyrimidine-3-carboxylic acid (80.0 mg, 0.418 mmol, CAS No. 90349-23-8), trans-4-amino-N-(3-chloropyridin-4-yl)cyclohexanecarboxamide hydrochloride (109 mg, 0.377 mmol) and N-ethyl-N-isopropylpropan-2-amine (292 μl, 1.67 mmol) in N,N-dimethylformamide (0.60 ml) and the mixture was stirred for 3 h at room temperature. For work-up, water was added, and the precipitate was collected by filtration. The crude product was recrystallized from methanol/water to yield the title compound (103 mg, 58% yield).

LC-MS (Method 1): Rₜ=0.95 min; MS (ESIpos) m/z=427.4 [M+H]⁺.

¹H-NMR (400 MHz, DMSO-d₆): δ [ppm]=9.64 (br. s, 1H), 8.60 (s, 1H), 8.50 (s, 1H), 8.42 (d, 1H), 8.06 (d, 1H), 8.01 (d, 1H), 7.13 (d, 1H), 3.88-3.72 (m, 1H), 2.78-2.69 (m, 4H), 2.63 (s, 3H), 2.14-2.06 (m, 2H), 2.02-1.90 (m, 2H), 1.68-1.50 (m, 2H), 1.45-1.29 (m, 2H).

### Example 103

### N-{trans-4-[(3-chloropyridin-4-yl)carbamoyl]cyclohexyl}imidazo[1,2-b]pyridazine-3-carboxamide

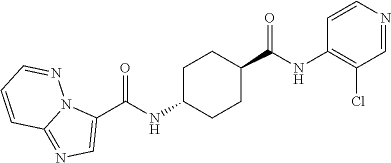 [SMILES: O=C(N[C@H]1CC[C@H](C(=O)Nc2ccncc2Cl)CC1)c1cnc2cccnn12]

Prepared in analogy to N-{trans-4-[(3-chloropyridin-4-yl)carbamoyl]cyclohexyl}-5,7-dimethylpyrazolo[1,5-a]pyrimidine-3-carboxamide using imidazo[1,2-b]pyridazine-3-carboxylic acid (68.2 mg, 0.418 mmol, CAS No. 1308384-58-8) as starting material. For work-up, water was added to the reaction mixture and the precipitate formed was collected by filtration, washed with water and dried to yield the title compound (151 mg, 90% yield).

LC-MS (Method 1): Rₜ=0.76 min; MS (ESIpos) m/z=399.2 [M+H]⁺.

¹H-NMR (400 MHz, DMSO-d₆): δ [ppm]=9.68 (br. s, 1H), 8.82-8.77 (m, 1H), 8.60 (s, 1H), 8.47 (d, 1H), 8.42 (d, 1H), 8.35 (dd, 1H), 8.31 (s, 1H), 8.05 (d, 1H), 7.50-7.44 (m, 1H), 3.94-3.79 (m, 1H), 2.76-2.63 (m, 1H), 2.15-1.94 (m, 4H), 1.67-1.52 (m, 2H), 1.51-1.36 (m, 2H).

### Example 104

### N-{trans-4-[(3-chloropyrid in-4-yl)carbamoyl]cyclohexyl}-6-methyl pyrazolo[1,5-a]pyrimidine-3-carboxamide

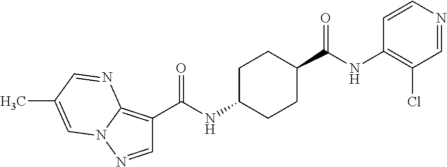 [SMILES: Cc1cnc2c(C(=O)N[C@H]3CC[C@H](C(=O)Nc4ccncc4Cl)CC3)cnn2c1]

(Benzotriazol-1-yloxy)tripyrrolidinophosphonium hexafluorophosphate (179 mg, 0.345 mmol) was added to a mixture of 6-methylpyrazolo[1,5-a]pyrimidine-3-carboxylic acid (55.5 mg, 0.313 mmol, CAS No. 869941-96-8), trans-4-amino-N-(3-chloropyridin-4-yl)cyclohexanecarboxamide hydrochloride (100 mg, 0.345 mmol) and N-ethyl-N-isopropylpropan-2-amine (218 μl, 1.25 mmol) in N,N-dimethylformamide (3.5 ml) and the mixture was stirred for 3 days at room temperature. For work-up, the reaction mixture was concentrated and the residue was washed with water and methanol. The crude product was purified by preparative HPLC (Method 9) followed by recrystallization from methanol to yield the title compound (9.8 mg, 8% yield).

LC-MS (Method 1): Rₜ=0.87 min; MS (ESIpos) m/z=413.2 [M+H]⁺.

¹H-NMR (400 MHz, DMSO-d₆): δ [ppm]=9.67 (br. s, 1H), 9.21-9.16 (m, 1H), 8.74 (d, 1H), 8.60 (s, 1H), 8.50 (s, 1H), 8.42 (d, 1H), 8.05 (d, 1H), 7.76 (d, 1H), 3.90-3.76 (m, 1H), 2.78-2.63 (m, 1H), 2.39 (d, 3H), 2.12-2.02 (m, 2H), 2.01-1.89 (m, 2H), 1.66-1.50 (m, 2H), 1.46-1.29 (m, 2H).

### Example 105

### N-{trans-4-[(3-chloropyridin-4-yl)carbamoyl]cyclohexyl}pyrazolo[1,5-a]pyrimidine-3-carboxamide

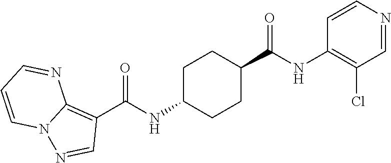 [SMILES: O=C(N[C@H]1CC[C@H](C(=O)Nc2ccncc2Cl)CC1)c1cnn2cccnc12]

1-Chlor-1-dimethylamino-2-methyl-1-propen (112 mg, 0.836 mmol) was added to a solution of pyrazolo[1,5-a]pyrimidine-3-carboxylic acid (68.2 mg, 0.418 mmol, CAS No. 25940-35-6) in dichloromethane (5.3 ml) and the mixture was stirred at room temperature for 30 min. Pyridine (101 μl, 1.25 mmol) and trans-4-amino-N-(3-chloropyridin-4-yl)cyclohexanecarboxamide hydrochloride (121 mg, 0.418 mmol) were added and the mixture was stirred over night at room temperature. For work-up, water was added and the mixture was extracted with dichloromethane. The organic phase was washed with water, dried and concentrated. The crude product was recrystallized from methanol to yield the title compound (30 mg, 18% yield).

LC-MS (Method 2): Rₜ=0.86 min; MS (ESIpos) m/z=399.2 [M+H]⁺.

¹H-NMR (400 MHz, DMSO-d₆): δ [ppm]=9.67 (br. s, 1H), 9.32 (dd, 1H), 8.83 (dd, 1H), 8.60 (s, 1H), 8.59 (s, 1H), 8.42 (d, 1H), 8.05 (d, 1H), 7.81 (d, 1H), 7.28 (dd, 1H), 3.90-3.76 (m, 1H), 2.71-2.64 (m, 1H), 2.12-1.90 (m, 4H), 1.65-1.49 (m, 2H), 1.46-1.31 (m, 2H).

### Example 106

### ethyl 3-({trans-4-[(2-chloro-4-fluorophenyl)carbamoyl]cyclohexyl}carbamoyl)pyrazolo[1,5-a]pyrimidine-6-carboxylate

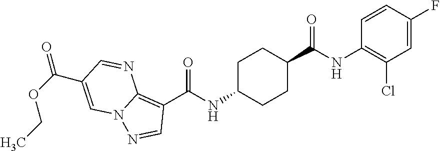 [SMILES: CCOC(=O)c1cnc2c(C(=O)N[C@H]3CC[C@H](C(=O)Nc4ccc(F)cc4Cl)CC3)cnn2c1]

(Benzotriazol-1-yloxy)tripyrrolidinophosphonium hexafluorophosphate (261 mg, 0.502 mmol) was added to a mixture of 6-(ethoxycarbonyl)pyrazolo[1,5-a]pyrimidine-3-carboxylic acid (1.00 g, 4.25 mmol) and N-ethyl-N-isopropylpropan-2-amine (3.0 ml, 17 mmol) in N,N-dimethylformamide (7.0 ml) followed by trans-4-amino-N-(2-chloro-4-fluorophenyl)cyclohexanecarboxamide hydrochloride (1.18 g, 3.83 mol) and N,N-dimethylformamide (1.0 ml) and the mixture was stirred for 2 h at room temperature. N,N-dimethylformamide (2.0 ml) was added and the mixture was stirred over night. Water was added and the precipitate formed was collected by filtration, washed with water and dried under vacuum at 50° C. to yield the title compound (1.32 g, 62% yield).

LC-MS (Method 2): Rₜ=1.19 min; MS (ESIpos) m/z=488.32 [M+H]⁺.

¹H-NMR (400 MHz, DMSO-d₆): δ [ppm]=9.78 (d, 1H), 9.53 (br. s, 1H), 9.15 (d, 1H), 8.76 (s, 1H), 7.80 (d, 1H), 7.60 (dd, 1H), 7.51 (dd, 1H), 7.26-7.18 (m, 1H), 4.41 (q, 2H), 3.92-3.76 (m, 1H), 2.12-1.92 (m, 4H), 1.67-1.51 (m, 2H), 1.48-1.32 (m, 5H).

### Example 107

### N-{trans-4-[(2-chloro-4-fluorophenyl)carbamoyl]cyclohexyl}-5-(morpholin-4-yl)pyrazolo[1,5-a]pyrimidine-3-carboxamide

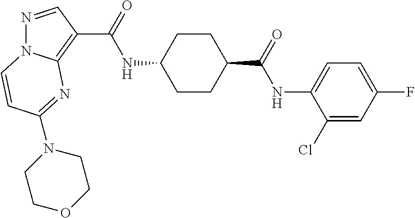 [SMILES: O=C(N[C@H]1CC[C@H](C(=O)Nc2ccc(F)cc2Cl)CC1)c1cnn2ccc(N3CCOCC3)nc12]

(benzotriazol-1-yloxy)tripyrrolidinophosphonium hexafluorophosphate (129 mg, 0.247 mmol) was added to a mixture of lithium 5-(morpholin-1-yl)pyrazolo[1,5-a]pyrimidine-3-carboxylate (68 mg, 84% purity, 0.225 mmol) and trans-4-amino-N-(2-chloro-4-fluorophenyl)cyclohexanecarboxamide hydrochloride (75.9 mg, 0.247 mmol) and N-ethyl-N-isopropylpropan-2-amine (196 μl, 1.12 mmol) in 2.5 ml N,N-dimethylformamide and the mixture was stirred over night at room temperature. For work-up, the reaction mixture was concentrated under reduced pressure and the residue purified by preparative HPLC (Method 9) followed by recrystallization from methanol to yield the title compound (32.5 mg, 29% yield).

LC-MS (Method 2): Rₜ=1.04 min; MS (ESIpos) m/z=501.3 [M+H]⁺.

1H-NMR (400 MHz, DMSO-d6): δ [ppm]=9.49 (br. s, 1H), 8.81 (d, 1H), 8.18 (s, 1H), 7.73-7.66 (m, 1H), 7.64-7.56 (m, 1H), 7.50 (dd, 1H), 7.22 (td, 1H), 6.89 (d, 1H), 3.87-3.59 (m, 9H), 2.14-2.01 (m, 2H), 2.00-1.86 (m, 2H), 1.66-1.47 (m, 2H), 1.38-1.20 (m, 2H).

### Example 108

### N-{trans-4-[(2-chloro-4-fluorophenyl)carbamoyl]cyclohexyl}-6-(pyrrolidin-1-yl)pyrazolo[1,5-a]pyrimidine-3-carboxamide

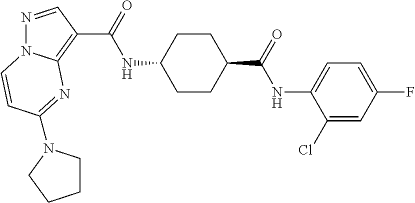 [SMILES: O=C(N[C@H]1CC[C@H](C(=O)Nc2ccc(F)cc2Cl)CC1)c1cnn2ccc(N3CCCC3)nc12]

(benzotriazol-1-yloxy)tripyrrolidinophosphonium hexafluorophosphate (160 mg, 0.308 mmol) was added to a mixture of lithium 5-(pyrrolidin-1-yl)pyrazolo[1,5-a]pyrimidine-3-carboxylate (92.6 mg, 72% purity, 0.280 mmol) and trans-4-amino-N-(2-chloro-4-fluorophenyl)cyclohexanecarboxamide hydrochloride (94.6 mg, 0.308 mmol) and N-ethyl-N-isopropylpropan-2-amine (244 μl, 1.40 mmol) in 3.1 ml N,N-dimethylformamide and the mixture was stirred over night at room temperature. For work-up, the reaction mixture was concentrated under reduced pressure and the residue purified by preparative HPLC (Method 9) to yield the title compound (17.5 mg, 13% yield).

LC-MS (Method 2): Rₜ=1.16 min; MS (ESIpos) m/z=485.3 [M+H]⁺.

¹H-NMR (400 MHz, DMSO-d₆): δ [ppm]=9.48 (s, 1H), 8.73 (d, 1H), 8.12 (s, 1H), 7.97 (d, 1H), 7.63 (dd, 1H), 7.50 (dd, 1H), 7.22 (td, 1H), 6.54 (d, 1H), 3.79-3.67 (m, 1H), 3.65-3.51 (m, 4H), 2.16-1.88 (m, 8H), 1.66-1.50 (m, 2H), 1.36-1.20 (m, 2H).

### Example 109

### N-{trans-4-[(2-chloro-4-fluorophenyl)carbamoyl]cyclohexyl}-6-(4-methylpiperazin-1-yl)pyrazolo[1,5-a]pyrimidine-3-carboxamide

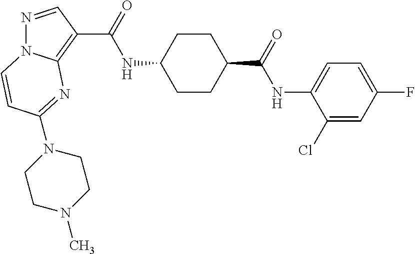 [SMILES: CN1CCN(c2ccn3ncc(C(=O)N[C@H]4CC[C@H](C(=O)Nc5ccc(F)cc5Cl)CC4)c3n2)CC1]

(benzotriazol-1-yloxy)tripyrrolidinophosphonium hexafluorophosphate (160 mg, 0.308 mmol) was added to a mixture of lithium 5-(4-methylpiperazin-1-yl)pyrazolo[1,5-a]pyrimidine-3-carboxylate (99.0 mg, 75% purity, 0.278 mmol) and trans-4-amino-N-(2-chloro-4-fluorophenyl)cyclohexanecarboxamide hydrochloride (93.9 mg, 0.306 mmol) and N-ethyl-N-isopropylpropan-2-amine (242 μl, 1.39 mmol) in 3.1 ml N,N-dimethylformamide and the mixture was stirred over night at room temperature. For work-up, the reaction mixture was concentrated under reduced pressure and the residue purified by preparative HPLC (Method 8) to yield the title compound (20.0 mg, 14% yield).

LC-MS (Method 2): Rₜ=1.02 min; MS (ESIpos) m/z=514.3 [M+H]⁺.

¹H-NMR (400 MHz, DMSO-d₆): δ [ppm]=9.46 (s, 1H), 9.01-8.80 (m, 1H), 8.22 (s, 1H), 7.75-7.56 (m, 2H), 7.54-7.44 (m, 1H), 7.30-7.15 (m, 1H), 7.04-6.81 (m, 1H), 4.84-4.05 (m, 1H), 3.82-3.68 (m, 1H), 2.19-1.87 (m, 4H), 1.70-1.51 (m, 2H), 1.42-1.15 (m, 2H).

### Example 110

### N-{trans-4-[(2-chloro-4-fluorophenyl)carbamoyl]cyclohexyl}-5-(methoxymethyl)-7-methylpyrazolo[1,5-a]pyrimidine-3-carboxamide

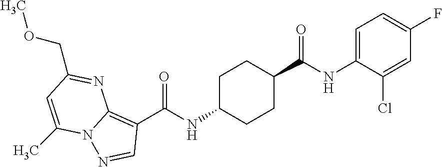 [SMILES: COCc1cc(C)n2ncc(C(=O)N[C@H]3CC[C@H](C(=O)Nc4ccc(F)cc4Cl)CC3)c2n1]

(benzotriazol-1-yloxy)tripyrrolidinophosphonium hexafluorophosphate (226 mg, 0.434 mmol) was added to a mixture of 5-(methoxymethyl)-7-methylpyrazolo[1,5-a]pyrimidine-3-carboxylic acid (isomer 1) and 7-(methoxymethyl)-5-methylpyrazolo[1,5-a]pyrimidine-3-carboxylic acid (isomer 2) (80 mg, 0.362 mmol) and trans-4-amino-N-(2-chloro-4-fluorophenyl)cyclohexanecarboxamide hydrochloride (100 mg, 0.325 mmol) and N-ethyl-N-isopropylpropan-2-amine (252 μl, 1.45 mmol) in 5.0 ml N,N-dimethylformamide and the mixture was stirred over night at room temperature. For work-up, water was added and the mixture was extracted with dichlormethane. The organic phase was filtrated through a silicone filter, concentrated and the residue was purified by preparative HPLC (Method 8) to give the title compound (50.0 mg) together with N-{trans-4-[(2-chloro-4-fluorophenyl)carbamoyl]cyclohexyl}-7-(methoxymethyl)-5-methylpyrazolo[1,5-a]pyrimidine-3-carboxamide (30.0 mg) (see Example 111).

LC-MS (Method 1): Rₜ=1.12 min; MS (ESIpos) m/z=474.4 [M+H]⁺.

¹H-NMR (400 MHz, DMSO-d₆): δ [ppm]=9.49 (s, 1H), 8.56 (s, 1H), 7.94 (d, 1H), 7.61 (dd, 1H), 7.50 (dd, 1H), 7.29-7.16 (m, 2H), 4.66 (s, 2H), 3.86-3.67 (m, 1H), 3.46 (s, 3H), 2.80 (s, 3H), 2.16-2.02 (m, 2H), 2.01-1.91 (m, 2H), 1.68-1.49 (m, 2H), 1.43-1.28 (m, 2H).

### Example 111

### N-{trans-4-[(2-chloro-4-fluorophenyl)carbamoyl]cyclohexyl}-7-(methoxymethyl)-5-methylpyrazolo[1,5-a]pyrimidine-3-carboxamide

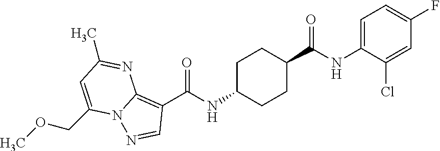 [SMILES: COCc1cc(C)nc2c(C(=O)N[C@H]3CC[C@H](C(=O)Nc4ccc(F)cc4Cl)CC3)cnn12]

Isolated as product in the synthesis of N-{trans-4-[(2-chloro-4-fluorophenyl)carbamoyl]cyclohexyl}-5-(methoxymethyl)-7-methylpyrazolo[1,5-a]pyrimidine-3-carboxamide (Example 110).

LC-MS (Method 1): Rₜ=1.17 min; MS (ESIpos) m/z=474.4 [M+H]⁺.

¹H-NMR (400 MHz, DMSO-d₆): δ [ppm]=9.49 (s, 1H), 8.50 (s, 1H), 7.96 (d, 1H), 7.61 (dd, 1H), 7.50 (dd, 1H), 7.22 (td, 1H), 7.16 (br. s, 1H), 4.96 (d, 2H), 3.88-3.69 (m, 1H), 3.52 (s, 3H), 2.68 (s, 3H), 2.13-1.86 (m, 4H), 1.68-1.48 (m, 2H), 1.45-1.27 (m, 2H).

### Example 112

### N-{trans-4-[(2-chloro-4,6-difluorophenyl)carbamoyl]cyclohexyl}-5-(methoxymethyl)-7-methylpyrazolo[1,5-a]pyrimidine-3-carboxamide

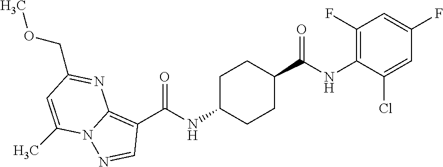 [SMILES: COCc1cc(C)n2ncc(C(=O)N[C@H]3CC[C@H](C(=O)Nc4c(F)cc(F)cc4Cl)CC3)c2n1]

Was prepared in analogy to the synthesis of N-{trans-4-[(2-chloro-4-fluorophenyl)carbamoyl]cyclohexyl}-5-(methoxymethyl)-7-methylpyrazolo[1,5-a]pyrimidine-3-carboxamide trans-4-amino-N-(2-chloro-4,6-difluorophenyl)cyclohexanecarboxamide hydrochloric acid salt (37.0 mg, 114 μmol) as starting material to give the title compound (41.0 mg).

LC-MS (Method 1): Rₜ=1.07 min; MS (ESIpos) m/z=492.3 [M+H]⁺.

¹H-NMR (400 MHz, DMSO-d₆): δ [ppm]=9.61 (s, 1H), 8.55 (s, 1H), 7.95 (d, 1H), 7.50-7.37 (m, 2H), 7.23 (br. s, 1H), 4.66 (s, 2H), 3.87-3.70 (m, 1H), 3.46 (s, 3H), 2.80 (s, 3H), 2.47-2.40 (m, 1H), 2.13-1.88 (m, 4H), 1.67-1.51 (m, 2H), 1.43-1.30 (m, 2H).

### Example 113

### N-{trans-4-[(2-chloro-4-fluorophenyl)carbamoyl]cyclohexyl}-5-methyl-7,8-dihydro-6H-cyclopenta[e]pyrazolo[1,5-a]pyrimidine-3-carboxamide

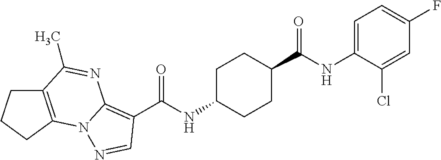 [SMILES: Cc1nc2c(C(=O)N[C@H]3CC[C@H](C(=O)Nc4ccc(F)cc4Cl)CC3)cnn2c2c1CCC2]

(benzotriazol-1-yloxy)tripyrrolidinophosphonium hexafluorophosphate (172 mg, 0.331 mmol) was added to a mixture of 5-methyl-7,8-dihydro-6H-cyclopenta[e]pyrazolo[1,5-a]pyrimidine-3-carboxylic acid (major isomer) and 8-methyl-6,7-dihydro-5H-cyclopenta[d]pyrazolo[1,5-a]pyrimidine-3-carboxylic acid (minor isomer) (60.0 mg, 0.276 mmol, ratio of isomers: 10/1), trans-4-amino-N-(2-chloro-4-fluorophenyl)cyclohexanecarboxamide hydrochloride (76.4 mg, 0.249 mmol) and 192 μl (1.11 mmol)N-ethyl-N-isopropylpropan-2-amine in 2.0 ml N,N-dimethylformamide and the mixture was stirred over night at room temperature. For work-up, water was added and the precipitate was collected by filtration, washed with water and dried under vacuum at 50° C. to provide the title compound (103 mg) together with its isomer N-{trans-4-[(2-chloro-4-fluorophenyl)carbamoyl]cyclohexyl}-8-methyl-6,7-dihydro-5H-cyclopenta[d]pyrazolo[1,5-a]pyrimidine-3-carboxamide (ratios of isomers ca. 10/1 by 1H NMR). The two isomers were separated by preparative HPLC [Instrument: Labomatic Pumpe HD-5000, Labomatic SP-3000, Labocord 5000, Labomatic Labcol Vario 4000, Gilson GX-241; Column: Chiralpak IE 5 μm 250×30 mm Nr.027; Solvent: ethanol/methanol/diethylamine 50:50:0.1 (v/v/v), flow: 30 ml/min; temperature: room temperature; MWD 254 nm] to give N-{trans-4-[(2-chloro-4-fluorophenyl)carbamoyl]cyclohexyl}-5-methyl-7,8-dihydro-6H-cyclopenta[e]pyrazolo[1,5-a]pyrimidine-3-carboxamide (76.0 mg, 57% yield). The minor isomer was not isolated in pure form.

LC-MS (Method 2): Rₜ=1.23 min; MS (ESIpos) m/z=470.1 [M+H]⁺.

¹H-NMR (400 MHz, DMSO-d₆): δ [ppm]=9.51 (br. s, 1H), 8.47 (s, 1H), 8.05 (d, 1H), 7.62 (dd, 1H), 7.51 (dd, 1H), 7.23 (td, 1H), 3.87-3.72 (m, 1H), 3.40-3.36 (m, 2H), 3.07-3.01 (m, 2H), 2.60 (s, 3H), 2.32-2.22 (m, 2H), 2.15-2.06 (m, 2H), 2.02-1.93 (m, 2H), 1.68-1.50 (m, 2H), 1.47-1.32 (m, 2H).

### Example 114

### N-{trans-4-[(2-chloro-4-fluorophenyl)carbamoyl]cyclohexyl}-6-[(2-hydroxypropyl)amino]imidazo[1,2-b]pyridazine-3-carboxamide

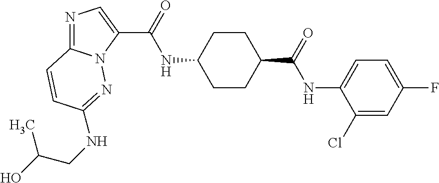 [SMILES: CC(O)CNc1ccc2ncc(C(=O)N[C@H]3CC[C@H](C(=O)Nc4ccc(F)cc4Cl)CC3)n2n1]

Was prepared in analogy to N-{trans-4-[(2-chloro-4-fluorophenyl)carbamoyl]cyclohexyl}-6-(pyrrolidin-1-yl)imidazo[1,2-b]pyridazine-3-carboxamide using (rac)-1-aminopropan-2-ol (25.0 mg, 0.333 mmol) as starting material. The crude mixture was purified twice by preparative HPLC to give the title compound (15.0 mg, 14% yield).

LC-MS (Method 1): t=0.92 min; MS (ESIpos) m/z=489.3 [M+H]⁺.

¹H-NMR (400 MHz, DMSO-d₆, rotamers): δ [ppm]=9.58-9.46 (m, 1H), 8.83 (d, 0.7H), 8.79 (d, 0.2H), 7.90-7.82 (m, 2H), 7.63-7.54 (m, 1.7H), 7.53-7.47 (m, 1H), 7.44-7.39 (m, 0.3H), 7.27-7.18 (m, 1H), 6.99 (d, 0.3H), 6.92 (d, 0.7H), 4.91 (d, 0.7H), 4.80 (d, 0.3H), 4.17-3.72 (m, 2H), 3.29-3.04 (m, 2H), 2.18-2.07 (m, 1.5H), 2.01-1.50 (m, 5H), 1.45-1.14 (m, 4.5H), 1.07 (d, 0.6H).

### Example 115

### N⁴-{4-[(2-chloro-4-fluorophenyl)carbamoyl]bicyclo[2.2.2]oct-1-yl}-N⁵-methyl-1H-imidazole-4,5-dicarboxamide

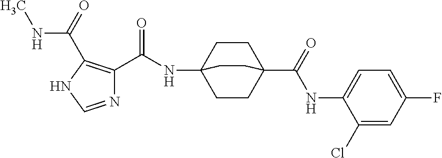 [SMILES: CNC(=O)c1[nH]cnc1C(=O)NC12CCC(C(=O)Nc3ccc(F)cc3Cl)(CC1)CC2]

Trimethylaluminum (0.60 ml, 1.2 mmol, 2 M solution in toluene) was added at 0° C. to a suspension of 2-chloro-4-fluoroaniline (174 mg, 1.20 mmol, Cas No 2106-02-7) in toluene (2.5 ml) and the mixture was stirred at room temperature until no further gas evolution was detected. A solution of methyl 4-({[5-(methylcarbamoyl)-1H-imidazol-4-yl]carbonyl}amino)bicyclo[2.2.2]octane-1-carboxylate (100 mg, 0.239 mmol) was added ad the mixture was stirred for 4 h at 80° C. Upon cooling to 0° C. methanol (1.4 ml) was added dropwise and the mixture was stirred for 1 h at room temperature and then concentrated under vacuum. The crude product was purified by flash chromatography (Snap Cartridge, hexanes/ethyl acetate gradient followed by ethyl acetate/methanol 9:1) followed by preparative HPLC [Waters Autopurificationsystem: Pump 254, Sample Manager 2767, CFO; DAD 2996, SQD 3100; XBrigde C18 5 μm 100×30 mm; eluent A=water+0.1% formic acid (99%); B=acetonitrile; 0-0.5 min 25 ml/min to 70 ml/min 33% B; 0.5-5.5 min 33-53% B; 70 ml/min; room temperature; injection: 25 mg/2 ml; 2×1 ml; DAD scan range 210-400 nm; MS ESI+, ESI−, scan range 160-1000 m/z] to yield the title compound (7.7 mg, 7% yield)

LC-MS (Method 4): Rₜ=1.08 min; MS (ESIneg) m/z=446.2 [M−H]⁻.

¹H-NMR (400 MHz, DMSO-d₆): δ [ppm]=13.42-12.31 (m, 1H), 11.19-11.00 (m, 1H), 9.02 (s, 1H), 8.74-8.45 (m, 1H), 7.92-7.72 (m, 1H), 7.51 (dd, 1H), 7.47-7.42 (m, 1H), 7.22 (td, 1H), 2.87-2.73 (m, 3H), 2.06-1.89 (m, 12H).

### Example 116

### N⁴-{4-[(2-chloro-4,6-difluorophenyl)carbamoyl]bicyclo[2.2.2]oct-1-yl}-N⁵-methyl-1H-imidazole-4,5-dicarboxamide

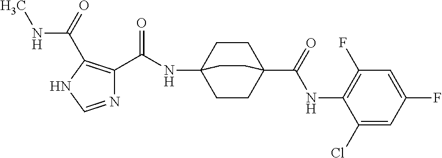 [SMILES: CNC(=O)c1[nH]cnc1C(=O)NC12CCC(C(=O)Nc3c(F)cc(F)cc3Cl)(CC1)CC2]

1-Chlor-1-dimethylamino-2-methyl-1-propen (97 μl, 800 μmol) was added to a solution of 4-({[5-(methylcarbamoyl)-1H-imidazol-4-yl]carbonyl}amino)bicyclo[2.2.2]octane-1-carboxylic acid (85.0 mg, 265 μmol) in dichloromethane (5.0 ml) and the mixture was stirred at room temperature for 30 min. Pyridine (110 μl, 1.3 mmol) and 2-chloro-4,6-difluoroaniline (86.8 mg, 531 μmol, CAS No. 36556-56-6) were added and the mixture was stirred over night at room temperature followed by 4 h at 40° C. For work-up, the reaction mixture was concentrated and the residue was purified by preparative HPLC to give the title compound 15.0 mg (97% purity, 12% yield).

LC-MS (Method 4): Rₜ=1.00 min; MS (ESIpos): m/z=466.2 [M+H]⁺

¹H-NMR (400 MHz, DMSO-d₆): δ [ppm]=13.08 (br. s., 1H), 11.33-10.77 (m, 1H), 9.14 (s, 1H), 8.82-8.11 (m, 1H), 7.75 (s, 1H), 7.51-7.35 (m, 2H), 2.79 (d, 3H), 2.07-1.85 (m, 12H).

### Example 117

### N-{4-[(2-chloro-4-fluorophenyl)carbamoyl]bicyclo[2.2.2]oct-1-yl}pyrazolo[1,5-a]pyrimidine-3-carboxamide

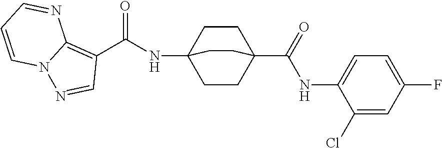 [SMILES: O=C(NC12CCC(C(=O)Nc3ccc(F)cc3Cl)(CC1)CC2)c1cnn2cccnc12]

1-Chlor-1-dimethylamino-2-methyl-1-propen (35.7 mg, 0.267 mmol) was added to a 4-[(pyrazolo[1,5-a]pyrimidin-3-ylcarbonyl)amino]bicyclo[2.2.2]octane-1-carboxylic acid (28.0 mg, 0.089 mmol) in dichloromethane (1.7 ml) and the mixture was stirred at room temperature for 30 min. Pyridine (36 μl, 0.45 mmol) and 2-chloro-4-fluoroaniline (25.9 mg, 0.178 mmol, Cas No 2106-02-7) were added and the mixture was stirred over night at room temperature. For work-up water was added and the mixture was extracted with a mixture of dichloromethane and 2-propanol (4:1). The combined organic phases were washed with saturated sodium bicarbonate solution and water, filtrated through a silicone filter and concentrated. The crude product purified by preparative HPLC (Method 9) to yield the title compound (27.5 mg, 95% purity, 66% yield).

LC-MS (Method 3): Rₜ=1.10 min; MS (ESIpos) m/z=442.1 [M+H]⁺.

¹H-NMR (500 MHz, DMSO-d₆): δ [ppm]=9.32 (dd, 1H), 9.05 (s, 1H), 8.82-8.80 (m, 1H), 8.53 (s, 1H), 7.71 (s, 1H), 7.51 (dd, 1H), 7.44 (dd, 1H), 7.29-7.25 (m, 1H), 7.22 (td, 1H), 2.08-1.91 (m, 12H).

### Example 118

### N-{4-[(2-chloro-4-fluoro-5-methylphenyl)carbamoyl]bicyclo[2.2.2]oct-1-yl}pyrazolo[1,5-a]pyrimidine-3-carboxamide

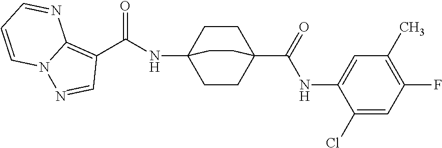 [SMILES: Cc1cc(NC(=O)C23CCC(NC(=O)c4cnn5cccnc45)(CC2)CC3)c(Cl)cc1F]

1-Chlor-1-dimethylamino-2-methyl-1-propen (70.1 mg, 0.525 mmol) was added to a 4-[(pyrazolo[1,5-a]pyrimidin-3-ylcarbonyl)amino]bicyclo[2.2.2]octane-1-carboxylic acid (55.0 mg, 0.175 mmol) in dichloromethane (3.3 ml) and the mixture was stirred at room temperature for 30 min. Pyridine (71 μl, 0.875 mmol) and 2-chloro-4-fluoro-5-methylaniline (55.8 mg, 0.350 mmol, CAS No. 124185-35-9) were added and the mixture was stirred over night at room temperature. For work-up the reaction mixture was concentrated and the residue was purified by preparative HPLC (Method 8) to yield the title compound (55.5 mg, 69% yield).

LC-MS (Method 1): Rₜ=1.20 min; MS (ESIpos) m/z=456.3 [M+H]⁺.

¹H-NMR (400 MHz, DMSO-d₆): δ [ppm]=9.32 (dd, 1H), 9.01 (s, 1H), 8.81 (dd, 1H), 8.53 (s, 1H), 7.71 (s, 1H), 7.42 (d, 1H), 7.34 (d, 1H), 7.27 (dd, 1H), 2.21 (d, 3H), 2.09-1.90 (m, 12H).

### Example 119

### N-{4-[(2-chloro-4,6-difluorophenyl)carbamoyl]bicyclo[2.2.2]oct-1-yl}pyrazolo[1,5-a]pyrimidine-3-carboxamide

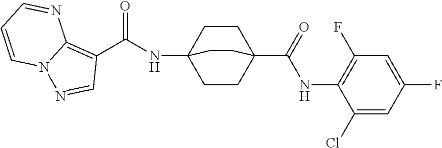 [SMILES: O=C(NC12CCC(C(=O)Nc3c(F)cc(F)cc3Cl)(CC1)CC2)c1cnn2cccnc12]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-[(pyrazolo[1,5-a]pyrimidin-3-ylcarbonyl)amino]bicyclo[2.2.2]octane-1-carboxylic acid (55.0 mg, 0.175 mmol) and 2-chloro-4,6-difluoroaniline (57.2 mg, 0.350 mmol, CAS No. 36556-56-6) to give the title compound (37.5 mg, 46% yield).

LC-MS (Method 1): Rₜ=1.03 min; MS (ESIpos) m/z=460.2 [M+H]⁺.

¹H-NMR (400 MHz, DMSO-d₆): δ [ppm]=9.32 (dd, 1H), 9.16 (br. s, 1H), 8.81 (dd, 1H), 8.53 (s, 1H), 7.71 (s, 1H), 7.49-7.38 (m, 2H), 7.30-7.24 (m, 1H), 2.09-1.90 (m, 12H)

### Example 120

### N-{4-[(4-chloropyrid in-3-yl)carbamoyl]bicyclo[2.2.2]oct-1-yl}pyrazolo[1,5-a]pyrimidine-3-carboxamide

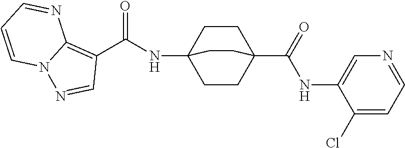 [SMILES: O=C(NC12CCC(C(=O)Nc3cnccc3Cl)(CC1)CC2)c1cnn2cccnc12]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-[(pyrazolo[1,5-a]pyrimidin-3-ylcarbonyl)amino]bicyclo[2.2.2]octane-1-carboxylic acid (55.0 mg, 0.175 mmol) and 4-chloropyridin-3-amine (45.0 mg, 0.350 mmol, Cas No. 20511-15-3) to give the title compound (38.5 mg, 51% yield).

LC-MS (Method 4): Rₜ=0.86 min; MS (ESIpos) m/z=425.2 [M+H]⁺.

¹H-NMR (400 MHz, DMSO-d₆): δ [ppm]=9.34-9.28 (m, 2H), 8.83-8.79 (m, 1H), 8.54 (s, 2H), 8.39 (d, 1H), 7.72 (s, 1H), 7.63 (d, 1H), 7.27 (dd, 1H), 2.13-1.91 (m, 12H).

### Example 121

### N-{4-[(2-chloro-4-fluorophenyl)carbamoyl]bicyclo[2.2.2]oct-1-yl}imidazo[1,2-b]pyridazine-3-carboxamide

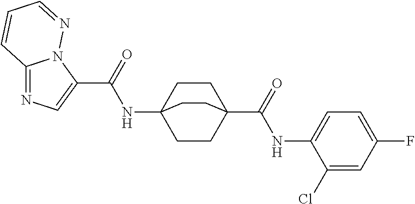 [SMILES: O=C(NC12CCC(C(=O)Nc3ccc(F)cc3Cl)(CC1)CC2)c1cnc2cccnn12]

Was prepared in analogy to N-{4-[(2-chloro-4-fluorophenyl)carbamoyl]bicyclo[2.2.2]oct-1-yl}pyrazolo[1,5-a]pyrimidine-3-carboxamide from 4-[(imidazo[1,2-b]pyridazin-3-ylcarbonyl)amino]bicyclo[2.2.2]octane-1-carboxylic acid (70.0 mg, 0.223 mmol) and 2-chloro-4-fluoroaniline (64.8 mg, 0.445 mmol, Cas No 2106-02-7) using preparative HPLC (Method 8) to give the title compound (60.0 mg, 60% yield).

LC-MS (Method 1): Rₜ=1.08 min; MS (ESIpos) m/z=442.2 [M+H]⁺.

¹H-NMR (400 MHz, DMSO-d₆): δ [ppm]=9.07 (br. s, 1H), 8.79 (dd, 1H), 8.39-8.33 (m, 2H), 8.27 (s, 1H), 7.54-7.41 (m, 3H), 7.22 (td, 1H), 2.14-1.93 (m, 12H).

### Example 122

### N-{4-[(2-chloro-4-fluoro-5-methylphenyl)carbamoyl]bicyclo[2.2.2]oct-1-yl}imidazo[1,2-b]pyridazine-3-carboxamide

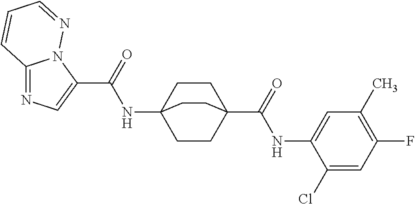 [SMILES: Cc1cc(NC(=O)C23CCC(NC(=O)c4cnc5cccnn45)(CC2)CC3)c(Cl)cc1F]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-[(imidazo[1,2-b]pyridazin-3-ylcarbonyl)amino]bicyclo[2.2.2]octane-1-carboxylic acid (70.0 mg, 0.223 mmol) and 2-chloro-4-fluoro-5-methylaniline (71.1 mg, 0.445 mmol, CAS No. 124185-35-9) using preparative HPLC (Method 8) to give the title compound (27.5 mg, 27% yield).

LC-MS (Method 1): Rₜ=1.16 min; MS (ESneg) m/z=454.2 [M−H]⁻.

¹H-NMR (400 MHz, DMSO-d₆): δ [ppm]=9.02 (s, 1H), 8.81-8.77 (m, 1H), 8.40-8.33 (m, 2H), 8.27 (s, 1H), 7.50-7.40 (m, 2H), 7.34 (d, 1H), 2.22 (d, 3H), 2.12-1.91 (m, 12H).

### Example 123

### N-{4-[(2-chloro-4,6-difluorophenyl)carbamoyl]bicyclo[2.2.2]oct-1-yl}imidazo[1,2-b]pyridazine-3-carboxamide

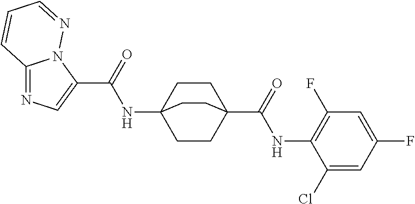 [SMILES: O=C(NC12CCC(C(=O)Nc3c(F)cc(F)cc3Cl)(CC1)CC2)c1cnc2cccnn12]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-[(imidazo[1,2-b]pyridazin-3-ylcarbonyl)amino]bicyclo[2.2.2]octane-1-carboxylic acid (70.0 mg, 0.223 mmol) and 2-chloro-4,6-difluoroaniline (72.8 mg, 0.445 mmol, CAS No. 36556-56-6) using preparative HPLC (Method 8) to give the title compound (55.5 mg, 54% yield).

LC-MS (Method 1): Rₜ=1.00 min; MS (ESIneg) m/z=458.2 [M−H]⁻.

¹H-NMR (400 MHz, DMSO-d₆): δ [ppm]=9.18 (s, 1H), 8.79 (dd, 1H), 8.41-8.32 (m, 2H), 8.27 (s, 1H), 7.52-7.37 (m, 3H), 2.14-1.89 (m, 12H).

### Example 124

### N-{4-[(4-chloropyrid in-3-yl)carbamoyl]bicyclo[2.2.2]oct-1-yl}imidazo[1,2-b]pyridazine-3-carboxamide

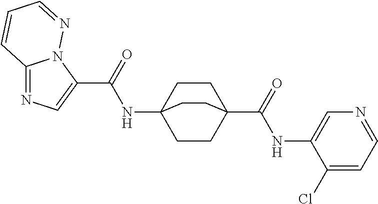 [SMILES: O=C(NC12CCC(C(=O)Nc3cnccc3Cl)(CC1)CC2)c1cnc2cccnn12]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-[(imidazo[1,2-b]pyridazin-3-ylcarbonyl)amino]bicyclo[2.2.2]octane-1-carboxylic acid (70.0 mg, 0.223 mmol) and 4-chloropyridin-3-amine (57.3 mg, 0.445 mmol, Cas No. 20511-15-3) using preparative HPLC (Method 8) to give the title compound (19.5 mg, 20% yield).

LC-MS (Method 4): Rₜ=0.82 min; MS (ESIneg) m/z=423.1 [M−H]⁻.

¹H-NMR (400 MHz, DMSO-d₆): δ [ppm]=9.32 (s, 1H), 8.79 (dd, 1H), 8.54 (s, 1H), 8.42-8.33 (m, 3H), 8.28 (s, 1H), 7.63 (d, 1H), 7.51-7.43 (m, 1H), 2.14-1.93 (m, 12H).

### Example 125

### N-{4-[(2-chloro-4-fluorophenyl)carbamoyl]bicyclo[2.2.2]oct-1-yl}-5,7-dimethylpyrazolo[1,5-a]pyrimidine-3-carboxamide

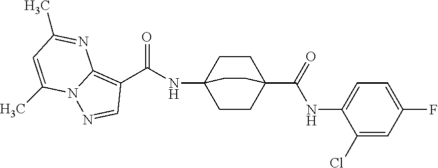 [SMILES: Cc1cc(C)n2ncc(C(=O)NC34CCC(C(=O)Nc5ccc(F)cc5Cl)(CC3)CC4)c2n1]

Was prepared in analogy to N-{4-[(2-chloro-4-fluorophenyl)carbamoyl]bicyclo[2.2.2]oct-1-yl}pyrazolo[1,5-a]pyrimidine-3-carboxamide from 4-{[(5,7-dimethylpyrazolo[1,5-a]pyrimidin-3-yl)carbonyl]amino}bicyclo[2.2.2]octane-1-carboxylic acid (70.0 mg, 2.04 mmol) and 2-chloro-4-fluoroaniline (59.5 mg, 0.409 mmol, Cas No 2106-02-7) using preparative HPLC (Method 8) to give the title compound (58.0 mg, 60% yield).

LC-MS (Method 1): Rₜ=1.25 min; MS (ESIneg) m/z=468.3 [M−H]⁻.

¹H-NMR (400 MHz, DMSO-d₆): δ [ppm]=9.05 (s, 1H), 8.45 (s, 1H), 7.96 (s, 1H), 7.51 (dd, 1H), 7.45 (dd, 1H), 7.27-7.18 (m, 1H), 7.12 (d, 1H), 2.74 (d, 3H), 2.61 (s, 3H), 2.12-1.92 (m, 12H).

### Example 126

### N-{4-[(2-chloro-4-fluoro-5-methylphenyl)carbamoyl]bicyclo[2.2.2]oct-1-yl}-5,7-dimethylpyrazolo[1,5-a]pyrimidine-3-carboxamide

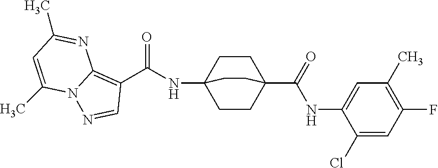 [SMILES: Cc1cc(C)n2ncc(C(=O)NC34CCC(C(=O)Nc5cc(C)c(F)cc5Cl)(CC3)CC4)c2n1]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-{[(5,7-dimethylpyrazolo[1,5-a]pyrimidin-3-yl)carbonyl]amino}bicyclo[2.2.2]octane-1-carboxylic acid (61.0 mg, 0.178 mmol) and 2-chloro-4-fluoro-5-methylaniline (56.9 mg, 0.356 mmol, CAS No. 124185-35-9) using preparative HPLC (Method 8) to give the title compound (29.5 mg, 34% yield).

LC-MS (Method 1): Rₜ=1.33 min; MS (ESIpos) m/z=484.3 [M+H]⁺.

¹H-NMR (400 MHz, DMSO-d₆): δ [ppm]=9.00 (s, 1H), 8.45 (s, 1H), 7.96 (s, 1H), 7.42 (d, 1H), 7.34 (d, 1H), 7.12 (d, 1H), 2.74 (d, 3H), 2.61 (s, 3H), 2.22 (d, 3H), 2.09-1.91 (m, 12H).

### Example 127

### N-{4-[(2-chloro-4,6-difluorophenyl)carbamoyl]bicyclo[2.2.2]oct-1-yl}-5,7-dimethylpyrazolo[1,5-a]pyrimidine-3-carboxamide

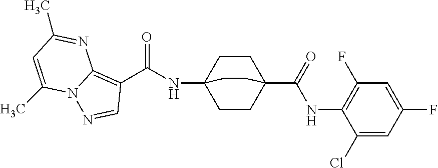 [SMILES: Cc1cc(C)n2ncc(C(=O)NC34CCC(C(=O)Nc5c(F)cc(F)cc5Cl)(CC3)CC4)c2n1]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-{[(5,7-dimethylpyrazolo[1,5-a]pyrimidin-3-yl)carbonyl]amino}bicyclo[2.2.2]octane-1-carboxylic acid (61.0 mg, 0.178 mmol) and 2-chloro-4,6-difluoroaniline (58.3 mg, 0.356 mmol, CAS No. 36556-56-6) using preparative HPLC (Method 8) to give the title compound (64.5 mg, 73% yield).

LC-MS (Method 1): Rₜ=1.17 min; MS (ESIpos) m/z=488.3 [M+H]⁺.

¹H-NMR (400 MHz, DMSO-d₆): δ [ppm]=9.16 (s, 1H), 8.45 (s, 1H), 7.95 (s, 1H), 7.51-7.37 (m, 2H), 7.12 (d, 1H), 2.74 (d, 3H), 2.61 (s, 3H), 2.08-1.91 (m, 12H).

### Example 128

### N-{4-[(4-chloropyrid in-3-yl)carbamoyl]bicyclo[2.2.2]oct-1-yl}-5,7-dimethylpyrazolo[1,5-a]pyrimidine-3-carboxamide

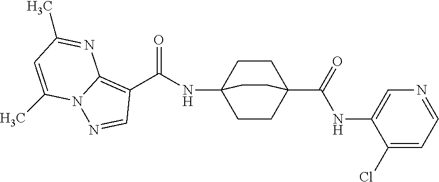 [SMILES: Cc1cc(C)n2ncc(C(=O)NC34CCC(C(=O)Nc5cnccc5Cl)(CC3)CC4)c2n1]

Was prepared in analogy to N-{4-[(2-chloro-4-fluoro-5-methylphenyl)carbamoyl]bicyclo[2.2.2]oct-1-yl}pyrazolo[1,5-a]pyrimidine-3-carboxamide from 4-{[(5,7-dimethylpyrazolo[1,5-a]pyrimidin-3-yl)carbonyl]amino}bicyclo[2.2.2]octane-1-carboxylic acid (86.0 mg, 0.251 mmol) and 4-chloropyridin-3-amine (64.6 mg, 0.502 mmol, Cas No. 20511-15-3) using preparative HPLC (Method 8) to give the title compound (20.5 mg, 18% yield).

LC-MS (Method 1): Rₜ=1.01 min; MS (ESIpos) m/z=453.3 [M+H]⁺.

¹H-NMR (400 MHz, DMSO-d₆): δ [ppm]=9.29 (br. s, 1H), 8.54 (s, 1H), 8.45 (s, 1H), 8.39 (d, 1H), 7.96 (s, 1H), 7.63 (d, 1H), 7.13 (d, 1H), 2.74 (s, 3H), 2.62 (s, 3H), 2.11-1.94 (m, 12H).

### Example 129

### N⁵-{trans-4-[(2-chloro-5-fluorophenyl)carbamoyl]cyclohexyl}-N⁴-(tetrahydrofuran-2-ylmethyl)-1H-imidazole-4,5-dicarboxamide

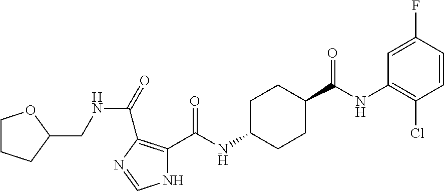 [SMILES: O=C(NCC1CCCO1)c1nc[nH]c1C(=O)N[C@H]1CC[C@H](C(=O)Nc2cc(F)ccc2Cl)CC1]

Phenyl 5-({trans-4-[(2-chloro-5-fluorophenyl)carbamoyl]cyclohexyl}carbamoyl)-1H-imidazole-4-carboxylate (100 mg, 0.206 mmol) and 1-(tetrahydrofuran-2-yl)methanamine (104 mg, 1.03 mmol, CAS No 4795-29-3) in tetrahydrofuran (5.2 ml) was stirred at 60° C. over night. For work-up the reaction mixture was concentrated and the crude product was recrystallized from methanol to yield the title compound (39.0 mg, 38% yield).

LC-MS (Method 6): Rₜ=1.09 min; MS (ESIpos) m/z=492.2 [M+H]⁺.

¹H-NMR (400 MHz, DMSO-d₆): δ [ppm]=13.22 (br. s., 1H), 11.41-10.91 (m, 1H), 9.54 (br. s., 1H), 8.58-8.26 (m, 1H), 7.82 (s, 1H), 7.73-7.66 (m, 1H), 7.58-7.50 (m, 1H), 7.11-7.02 (m, 1H), 4.08-3.87 (m, 1H), 3.85-3.57 (m, 3H), 3.42-3.35 (m, 2H), 2.62-2.54 (m, 1H), 2.16-1.72 (m, 7H), 1.64-1.14 (m, 5H).

### Example 130

### N-{trans-4-[(2-chloro-4,5-difluorophenyl)carbamoyl]cyclohexyl}pyrazolo[1,5-a]pyrimidine-3-carboxamide

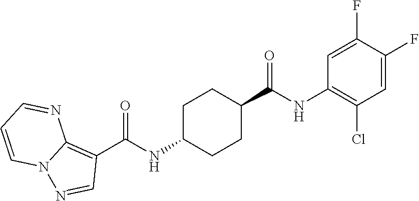 [SMILES: O=C(N[C@H]1CC[C@H](C(=O)Nc2cc(F)c(F)cc2Cl)CC1)c1cnn2cccnc12]

1-Chlor-1-dimethylamino-2-methyl-1-propen (64.9 mg, 0.486 mmol) was added to a solution of trans-4-[(pyrazolo[1,5-a]pyrimidin-3-ylcarbonyl)amino]cyclohexanecarboxylic acid (70.0 mg, 0.243 mmol) in dichloromethane (3.1 ml) and the mixture was stirred at room temperature for 30 min. Pyridine (59 μl, 0.0.73 mmol) and 2-chloro-4,5-difluoroaniline (39.7 mg, 0.243 mmol, CAS No 2613-32-3) were added and the mixture was stirred over night at room temperature. For work-up, water was added and the reaction mixture was extracted with dichloromethane. Die organic phase was filtrated through a silicone filter, concentrated and the residue purified by preparative HPLC (Method 9) to yield the title compound (3.8 mg, 4% yield).

LC-MS (Method 2): Rₜ=1.10 min; MS (ESIpos) m/z=434.1 [M+H]⁺.

¹H-NMR (400 MHz, DMSO-d₆): δ [ppm]=9.62 (s, 1H), 9.32 (dd, 1H), 8.83 (dd, 1H), 8.58 (s, 1H), 7.86-7.76 (m, 3H), 7.28 (dd, 1H), 3.90-3.76 (m, 1H), 2.12-1.91 (m, 4H), 1.66-1.50 (m, 2H), 1.45-1.31 (m, 2H).

### Example 131

### N-{trans-4-[(2-chloro-4,6-difluorophenyl)carbamoyl]cyclohexyl}pyrazolo[1,5-a]pyrimidine-3-carboxamide

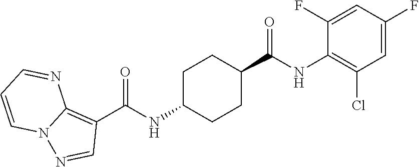 [SMILES: O=C(N[C@H]1CC[C@H](C(=O)Nc2c(F)cc(F)cc2Cl)CC1)c1cnn2cccnc12]

Was prepared in analogy to N-{trans-4-[(2-chloro-4,5-difluorophenyl)carbamoyl]cyclohexyl}pyrazolo[1,5-a]pyrimidine-3-carboxamide from trans-4-[(pyrazolo[1,5-a]pyrimidin-3-ylcarbonyl)amino]cyclohexanecarboxylic acid (70.0 mg, 0.243 mmol) and 2-chloro-4,6-difluoroaniline (39.7 mg, 0.243 mmol, CAS No. 36556-56-6) to give the title compound (1.2 mg, 1% yield).

LC-MS (Method 3): Rₜ=0.94 min; MS (ESIpos) m/z=434.1 [M+H]⁺.

¹H-NMR (400 MHz, DMSO-d₆): δ [ppm]=9.62 (s, 1H), 9.32 (dd, 1H), 8.83 (dd, 1H), 8.58 (s, 1H), 7.80 (d, 1H), 7.50-7.38 (m, 2H), 7.30-7.25 (m, 1H), 3.89-3.76 (m, 1H), 2.46-2.39 (m, 1H), 2.10-1.92 (m, 4H), 1.67-1.52 (m, 2H), 1.47-1.33 (m, 2H).

### Example 132

### N-{trans-4-[(2-chloro-4,5-difluorophenyl)carbamoyl]cyclohexyl}imidazo[1,2-b]pyridazine-3-carboxamide

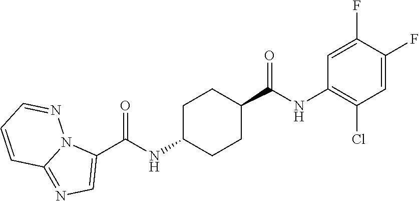 [SMILES: O=C(N[C@H]1CC[C@H](C(=O)Nc2cc(F)c(F)cc2Cl)CC1)c1cnc2cccnn12]

Was prepared in analogy to N-{trans-4-[(2-chloro-4,5-difluorophenyl)carbamoyl]cyclohexyl}pyrazolo[1,5-a]pyrimidine-3-carboxamide from trans-4-[(imidazo[1,2-b]pyridazin-3-ylcarbonyl)amino]cyclohexanecarboxylic acid (34.0 mg, 0.118 mmol) and 2-chloro-4,5-difluoroaniline (19.3 mg, 0.118 mmol, CAS No 2613-32-3). For work-up, water was added and the precipitate formed was collected by filtration, washed with dichloromethane and dried. The crude product was purified by preparative HPLC (Method 8) to give the title compound (12.0 mg, 23% yield).

LC-MS (Method 4): Rₜ=1.05 min; MS (ESIpos) m/z=434.1 [M+H]⁺.

¹H-NMR (400 MHz, DMSO-d₆): δ [ppm]=9.63 (s, 1H), 8.80 (dd, 1H), 8.49-8.43 (m, 1H), 8.35 (dd, 1H), 8.30 (s, 1H), 7.87-7.75 (m, 2H), 7.52-7.43 (m, 1H), 3.95-3.79 (m, 1H), 2.16-1.89 (m, 4H), 1.67-1.36 (m, 4H).

### Example 133

### N-{trans-4-[(2-chloro-4,6-difluorophenyl)carbamoyl]cyclohexyl}imidazo[1,2-b]pyridazine-3-carboxamide

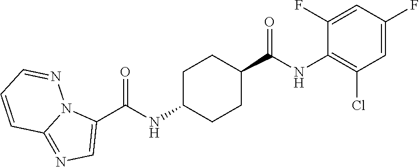 [SMILES: O=C(N[C@H]1CC[C@H](C(=O)Nc2c(F)cc(F)cc2Cl)CC1)c1cnc2cccnn12]

Was prepared in analogy to N-{trans-4-[(2-chloro-4,5-difluorophenyl)carbamoyl]cyclohexyl}pyrazolo[1,5-a]pyrimidine-3-carboxamide from trans-4-[(imidazo[1,2-b]pyridazin-3-ylcarbonyl)amino]cyclohexanecarboxylic acid (57.0 mg, 0.198 mmol) and 2-chloro-4,6-difluoroaniline (32.3 mg, 0.198 mmol, CAS No. 36556-56-6). For work-up, water was added and the precipitate formed was collected by filtration, washed with ethyl acetate and dried to give the title compound (61.0 mg, 70% yield).

LC-MS (Method 1): Rₜ=0.92 min; MS (ESIpos) m/z=434.2 [M+H]⁺.

¹H-NMR (400 MHz, DMSO-d₆): δ [ppm]=9.63 (s, 1H), 8.80 (dd, 1H), 8.47 (d, 1H), 8.35 (dd, 1H), 8.31 (s, 1H), 7.51-7.38 (m, 3H), 3.94-3.81 (m, 1H), 2.47-2.39 (m, 1H), 2.13-1.93 (m, 4H), 1.68-1.38 (m, 4H).

### Example 134

### N-{trans-4-[(2-chloro-4,5-difluorophenyl)carbamoyl]cyclohexyl}-5,7-dimethylpyrazolo[1,5-a]pyrimidine-3-carboxamide

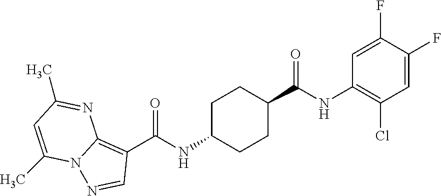 [SMILES: Cc1cc(C)n2ncc(C(=O)N[C@H]3CC[C@H](C(=O)Nc4cc(F)c(F)cc4Cl)CC3)c2n1]

Was prepared in analogy to N-{trans-4-[(2-chloro-4,5-difluorophenyl)carbamoyl]cyclohexyl}pyrazolo[1,5-a]pyrimidine-3-carboxamide from trans-4-{[(5,7-dimethylpyrazolo[1,5-a]pyrimidin-3-yl)carbonyl]amino}cyclohexanecarboxylic acid (70.0 mg, 0.221 mmol) and 2-chloro-4,5-difluoroaniline (36.2 mg, 221 mmol, CAS No 2613-32-3) to give the title compound (4.1 mg, 4% yield).

LC-MS (Method 3): Rₜ=1.18 min; MS (ESIpos) m/z=462.2 [M+H]⁺.

¹H-NMR (400 MHz, DMSO-d₆): δ [ppm]=9.60 (s, 1H), 8.50 (s, 1H), 8.00 (d, 1H), 7.88-7.75 (m, 2H), 7.13 (d, 1H), 3.87-3.72 (m, 1H), 2.76-2.72 (m, 3H), 2.63 (s, 3H), 2.15-1.89 (m, 4H), 1.68-1.50 (m, 2H), 1.45-1.30 (m, 2H).

### Example 135

### N-{trans-4-[(2-chloro-4,6-difluorophenyl)carbamoyl]cyclohexyl}-5,7-dimethylpyrazolo[1,5-a]pyrimidine-3-carboxamide

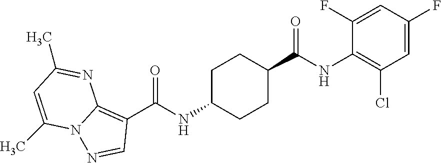 [SMILES: Cc1cc(C)n2ncc(C(=O)N[C@H]3CC[C@H](C(=O)Nc4c(F)cc(F)cc4Cl)CC3)c2n1]

Was prepared in analogy to N-{trans-4-[(2-chloro-4,5-difluorophenyl)carbamoyl]cyclohexyl}pyrazolo[1,5-a]pyrimidine-3-carboxamide from trans-4-{[(5,7-dimethylpyrazolo[1,5-a]pyrimidin-3-yl)carbonyl]amino}cyclohexanecarboxylic acid (70.0 mg, 0.221 mmol) and 2-chloro-4,6-difluoroaniline (36.2 mg, 0.221 mmol, CAS No. 36556-56-6) to give the title compound (11.2 mg, 11% yield).

LC-MS (Method 3): Rₜ=1.06 min; MS (ESIpos) m/z=462.2 [M+H]⁺.

¹H-NMR (400 MHz, DMSO-d₆): δ [ppm]=9.61 (s, 1H), 8.50 (s, 1H), 8.00 (d, 1H), 7.51-7.37 (m, 2H), 7.14-7.11 (m, 1H), 3.85-3.71 (m, 1H), 2.76-2.72 (m, 3H), 2.63 (s, 3H), 2.48-2.40 (m, 1H), 2.14-2.03 (m, 2H), 2.03-1.92 (m, 2H), 1.67-1.53 (m, 2H), 1.48-1.30 (m, 2H).

### Example 136

### N-[(trans)-2-(2-chloro-4-fluorophenyl)-1-oxo-2-azaspiro[4.5]dec-8-yl]pyrazolo[1,5-a]pyrimidine-3-carboxamide

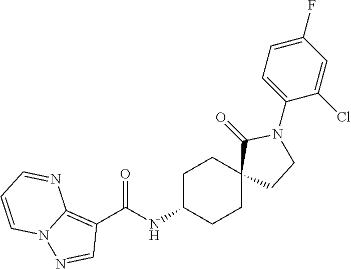 [SMILES: O=C(N[C@H]1CC[C@@]2(CCN(c3ccc(F)cc3Cl)C2=O)CC1)c1cnn2cccnc12]

(Benzotriazol-1-yloxy)tripyrrolidinophosphonium hexafluorophosphate (134 mg, 0.257 mmol) was added to a mixture of 8-amino-2-(2-chloro-4-fluorophenyl)-2-azaspiro[4.5]decan-1-one (isomer 1, intermediate I63) (69.4 mg, crude product), pyrazolo[1,5-a]pyrimidine-3-carboxylic acid (47.7 mg, 0.293 mmol, CAS No. 25940-35-6) and N-ethyl-N-isopropylpropan-2-amine (0.20 ml, 1.2 mmol) in N,N-dimethylformamide (2.6 ml) and the mixture was stirred for 12 h at room temperature. For work-up, the reaction mixture was concentrated under reduced pressure and the residue stirred with methanol, the precipitated was collected by filtration washed with water and ethanol and dried. The crude product was purified by flash chromatography (Snap cartridge, dichloromethane/methanol gradient) followed by preparative HPLC [Instrument: Waters Autopurificationsystem SQD; Column: Waters XBrigde C18 5μ 100×30 mm; Eluent A: water+0.1% Vol. formic acid (99%), Eluent B: acetonitrile; Gradient: 0.00-0.50 min 5% B (25-70 ml/min), 0.51-5.50 min 60% B (70 ml/min); Temperature: room temperature; DAD scan: 210-400 nm; MS ESIPos., scan range 160-1000 m/z] to yield the title compound (12.5 mg).

LC-MS (Method 1): Rₜ=1.04 min; MS (ESIpos) m/z=442.2 [M+H]⁺.

¹H-NMR (400 MHz, DMSO-d₆): δ [ppm]=9.33 (dd, 1H), 8.84 (dd, 1H), 8.59 (s, 1H), 7.83 (d, 1H), 7.62-7.57 (m, 1H), 7.49 (dd, 1H), 7.37-7.24 (m, 2H), 3.92-3.79 (m, 1H), 3.63 (t, 2H), 2.17 (t, 2H), 2.04-1.91 (m, 2H), 1.76-1.63 (m, 4H), 1.58-1.41 (m, 2H).

### Example 137

### N-[(trans)-2-(2-chloro-4-fluorophenyl)-1-oxo-2-azaspiro[4.5]dec-8-yl]-6-methylpyrazolo[1,5-a]pyrimidine-3-carboxamide

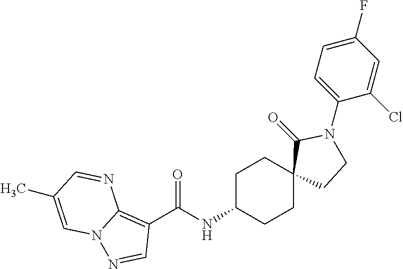 [SMILES: Cc1cnc2c(C(=O)N[C@H]3CC[C@@]4(CCN(c5ccc(F)cc5Cl)C4=O)CC3)cnn2c1]

(Benzotriazol-1-yloxy)tripyrrolidinophosphonium hexafluorophosphate (141 mg, 271 μmol) was added to a mixture of 8-amino-2-(2-chloro-4-fluorophenyl)-2-azaspiro[4.5]decan-1-one (isomer 1, intermediate I63) (73.0 mg, 246 μmol), 6-methylpyrazolo[1,5-a]pyrimidine-3-carboxylic acid (54.5 mg, 307 μmol) and N-ethyl-N-isopropylpropan-2-amine (210 μl, 1.2 mmol) in N,N-dimethylformamide (3.7 ml) and the mixture was stirred for 12 h at room temperature. For work-up, water was added and the precipitate formed was collected by filtration, washed with water and methanol and dried to give the title compound (78.0 mg, 98% purity, 68% yield).

LC-MS (Method 2): Rₜ=1.12 min; MS (ESIpos): m/z=456.3 [M+H]⁺

¹H-NMR (400 MHz, DMSO-d₆): δ [ppm]=9.21 (br. s., 1H), 8.96-8.40 (m, 2H), 8.01-7.65 (m, 1H), 7.59 (dd, 1H), 7.48 (dd, 1H), 7.31 (td, 1H), 3.91-3.73 (m, 1H), 3.62 (t, 2H), 2.46-2.35 (m, 3H), 2.16 (t, 2H), 2.04-1.87 (m, 2H), 1.74-1.61 (m, 4H), 1.57-1.37 (m, 2H).

### Example 138

### N-[(trans)-2-(2-chloro-4-fluorophenyl)-1-oxo-2-azaspiro[4.5]dec-8-yl]imidazo[1,2-b]pyridazine-3-carboxamide

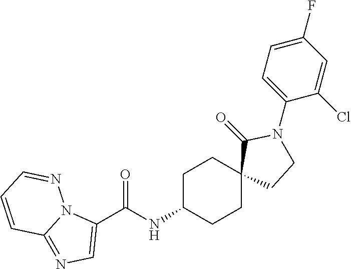 [SMILES: O=C(N[C@H]1CC[C@@]2(CCN(c3ccc(F)cc3Cl)C2=O)CC1)c1cnc2cccnn12]

(Benzotriazol-1-yloxy)tripyrrolidinophosphonium hexafluorophosphate (216 mg, 415 μmol) was added to a mixture of 8-amino-2-(2-chloro-4-fluorophenyl)-2-azaspiro[4.5]decan-1-one (isomer 1, intermediate I63) (160 mg, 70% purity, 377 μmol), imidazo[1,2-b]pyridazine-3-carboxylic acid (77.0 mg, 472 μmol, CAS No 1308384-58-8) and N-ethyl-N-isopropylpropan-2-amine (330 μl, 1.9 mmol) in N,N-dimethylformamide (5.6 ml) and the mixture was stirred for 12 h at room temperature. For work-up, water was added and the precipitate formed was collected by filtration, washed with water and methanol and dried to give the title compound (92.6 mg, 98% purity, 54% yield).

LC-MS (Method 2): Rₜ=1.03 min; MS (ESIpos): m/z=442.4 [M+H]⁺

¹H-NMR (400 MHz, DMSO-d₆): δ [ppm]=8.79 (dd, 1H), 8.48 (d, 1H), 8.34 (dd, 1H), 8.30 (s, 1H), 7.58 (dd, 1H), 7.52-7.42 (m, 2H), 7.31 (td, 1H), 3.95-3.83 (m, 1H), 3.63 (t, 2H), 2.16 (t, 2H), 2.04-1.93 (m, 2H), 1.76-1.63 (m, 4H), 1.62-1.45 (m, 2H).

### Example 139

### N-[(trans)-2-(2-chloro-4-fluorophenyl)-1-oxo-2-azaspiro[4.5]dec-8-yl]-5,7-dimethylpyrazolo[1,5-a]pyrimidine-3-carboxamide

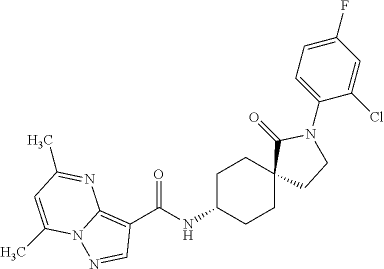 [SMILES: Cc1cc(C)n2ncc(C(=O)N[C@H]3CC[C@@]4(CCN(c5ccc(F)cc5Cl)C4=O)CC3)c2n1]

(Benzotriazol-1-yloxy)tripyrrolidinophosphonium hexafluorophosphate (216 mg, 415 μmol) was added to a mixture of 8-amino-2-(2-chloro-4-fluorophenyl)-2-azaspiro[4.5]decan-1-one (isomer 1, intermediate I63) (160 mg, 70% purity, 377 μmol), 5,7-dimethylpyrazolo[1,5-a]pyrimidine-3-carboxylic acid (90.2 mg, 472 μmol, CAS no 90349-23-8) and N-ethyl-N-isopropylpropan-2-amine (330 μl, 1.9 mmol) in N,N-dimethylformamide (5.6 ml) and the mixture was stirred for 12 h at room temperature. For work-up, water was added and the precipitate formed was collected by filtration, washed with water and methanol and dried. Dimethylsulfoxide was added; the precipitated formed was collected by filtration and then purified by flash chromatography (10 g Snap Cartridge, dichloromethane/methanol 95:5) to give the title compound (18.0 mg, 98% purity, 10% yield).

LC-MS (Method 2): Rₜ=1.20 min; MS (ESIpos): m/z=470.30 [M+H]⁺

¹H-NMR (400 MHz, DMSO-d₆): δ [ppm]=8.50 (s, 1H), 8.02 (d, 1H), 7.59 (dd, 1H), 7.49 (dd, 1H), 7.31 (td, 1H), 7.13 (d, 1H), 3.86-3.76 (m, 1H), 3.63 (t, 2H), 2.74 (s, 3H), 2.63 (s, 3H), 2.17 (t, 2H), 2.07-1.95 (m, 2H), 1.77-1.62 (m, 4H), 1.58-1.39 (m, 2H).

### Example 140

### N⁴-[(trans)-2-(2-chloro-4-fluorophenyl)-1-oxo-2-azaspiro[4.5]dec-8-yl]-N⁵-methyl-1H-imidazole-4,5-dicarboxamide

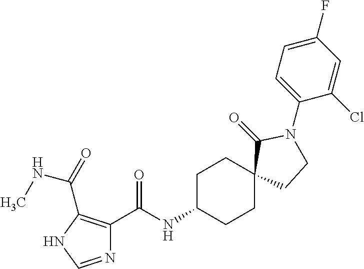 [SMILES: CNC(=O)c1[nH]cnc1C(=O)N[C@H]1CC[C@@]2(CCN(c3ccc(F)cc3Cl)C2=O)CC1]

8-Amino-2-(2-chloro-4-fluorophenyl)-2-azaspiro[4.5]decan-1-one (isomer 1, intermediate I63) (73.0 mg, crude product), was added to a suspension of N,N′-dimethyl-5,10-dioxo-5H,10H-diimidazo[1,5-a:1′,5′-d]pyrazine-1,6-dicarboxamide (37.2 mg, 1.23 mmol,) and triethylamine (69 μl, 0.49 mmol) in dichloromethane (3.1 ml) and the mixture was stirred for 24 h at room temperature. For work-up solids were filtrated of, the filtrate was concentrated under reduced pressure and the residue was purified by preparative HPLC (Method 8) to give the title compound (15.0 mg).

LC-MS (Method 1): Rₜ=1.02 min; MS (ESIpos) m/z=448.2 [M+H]⁺.

¹H-NMR (400 MHz, DMSO-d₆): δ [ppm]=13.71-12.45 (m, 0.6H), 11.92-10.24 (m, 0.3H), 9.23-8.15 (m, 0.5H), 7.79 (s, 1H), 7.60 (dd, 1H), 7.49 (dd, 1H), 7.32 (td, 1H), 3.82-3.69 (m, 1H), 3.63 (t, 2H), 2.82 (d, 3H), 2.15 (t, 2H), 2.00-1.81 (m, 2H), 1.79-1.60 (m, 4H), 1.59-1.34 (m, 2H).

### Example 141

### N⁴-[(trans)-2-(2-chloro-4-fluorophenyl)-1-oxo-2-azaspiro[4.5]dec-8-yl]-N⁵-ethyl-1H-imidazole-4,5-dicarboxamide

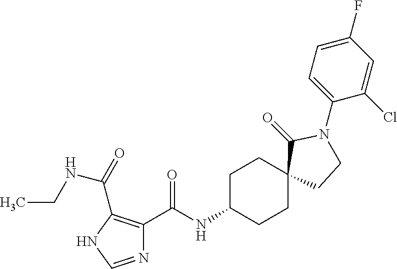 [SMILES: CCNC(=O)c1[nH]cnc1C(=O)N[C@H]1CC[C@@]2(CCN(c3ccc(F)cc3Cl)C2=O)CC1]

### Step 1:

A mixture of 8-Amino-2-(2-chloro-4-fluorophenyl)-2-azaspiro[4.5]decan-1-one (isomer 1, intermediate I63) (415 mg, crude product), diphenyl 5,10-dioxo-5H,10H-diimidazo[1,5-a:1′,5′-d]pyrazine-1,6-dicarboxylate (290 mg, 678 μmol) and triethylamine (0.23 ml) in tetrahydrofuran (21 ml) was stirred for 2.5 h at 60° C. Upon cooling, insoluble materials were filtrated off and the filtrated was concentrated to give phenyl 4-{[2-(2-chloro-4-fluorophenyl)-1-oxo-2-azaspiro[4.5]dec-8-yl]carbamoyl}-1H-imidazole-5-carboxylate (isomer 1) (749 mg) which was used in the subsequent step without further purification.

### Step 2:

Ethylamine (1.8 ml, 2.0 M solution in tetrahydrofuran, 3.7 mmol) was added to a solution of phenyl 4-{[2-(2-chloro-4-fluorophenyl)-1-oxo-2-azaspiro[4.5]dec-8-yl]carbamoyl}-1H-imidazole-5-carboxylate (isomer 1) (375 mg, 734 μmol) in tetrahydrofuran (5.0 ml) and the mixture was stirred for 24 h at room temperature. For work-up, insoluble material was filtrated off, the filtrate was concentrated and the residue was purified by preparative HPLC (Method 9) to give the title compound (66.0 mg).

LC-MS (Method 2): Rₜ=1.08 min; MS (ESIpos): m/z=462.4 [M+H]⁺

¹H-NMR (400 MHz, DMSO-d₆): δ [ppm]=13.19 (br. s., 1H), 11.71-10.49 (m, 1H), 9.63-8.50 (m, 1H), 7.80 (s, 1H), 7.59 (dd, 1H), 7.48 (dd, 1H), 7.31 (td, 1H), 3.85-3.68 (m, 1H), 3.62 (t, 2H), 3.31-3.25 (m, 2H), 2.14 (t, 2H), 2.01-1.77 (m, 2H), 1.72-1.58 (m, 4H), 1.57-1.38 (m, 2H), 1.13 (t, 3H)

### Example 142

### N⁵-(trans-4-{[2-chloro-5-(trifluoromethyl)phenyl]carbamoyl}cyclohexyl)-N⁴-methyl-1H-imidazole-4,5-dicarboxamide

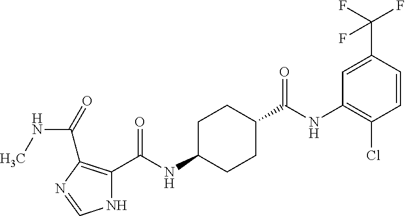 [SMILES: CNC(=O)c1nc[nH]c1C(=O)N[C@H]1CC[C@H](C(=O)Nc2cc(C(F)(F)F)ccc2Cl)CC1]

A mixture of phenyl 5-[(trans-4-{[2-chloro-5-(trifluoromethyl)phenyl]carbamoyl}cyclohexyl)carbamoyl]-1H-imidazole-4-carboxylate (100 mg, 0.187 mmol,) and methanamine (0.47 ml, 0.94 mmol, 2 M solution in tetrahydrofuran) in tetrahydrofuran (5.0 ml) was stirred at room temperature over night. For work-up, the reaction mixture was concentrated and the crude product was stirred with methanol. The precipitate was collected by filtration, and dried to yield the title compound (49.0 mg, 55% yield).

LC-MS (Method 6): Rₜ=1.11 min; MS (ESIpos) m/z=472.1 [M+H]⁺.

¹H-NMR (400 MHz, DMSO-d₆): δ [ppm]=13.21 (br. s., 1H), 11.65-10.89 (m, 1H), 9.72 (s, 1H), 8.73-8.30 (m, 1H), 8.15 (d, 1H), 7.85-7.78 (m, 1H), 7.75 (d, 1H), 7.54 (dd, 1H), 3.74 (br. s., 1H), 2.81 (d, 3H), 2.62-2.53 (m, 1H), 2.14-1.88 (m, 4H), 1.64-1.25 (m, 4H).

### Example 143

### N⁵-(trans-4-{[2-chloro-5-(trifluoromethyl)phenyl]carbamoyl}cyclohexyl)-N⁴-(1-methoxypropan-2-yl)-1H-imidazole-4,5-dicarboxamide

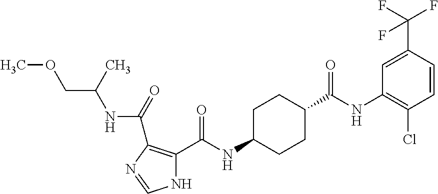 [SMILES: COCC(C)NC(=O)c1nc[nH]c1C(=O)N[C@H]1CC[C@H](C(=O)Nc2cc(C(F)(F)F)ccc2Cl)CC1]

A mixture of phenyl 5-[(trans-4-{[2-chloro-5-(trifluoromethyl)phenyl]carbamoyl}cyclohexyl)carbamoyl]-1H-imidazole-4-carboxylate (100 mg, 0.187 mmol,) and 1-methoxypropan-2-amine (83 mg, 0.935 mmol, CAS No 37143-54-7) in tetrahydrofuran (0.42 ml) was stirred at 60° C. over night. For work-up, the reaction mixture was concentrated and the crude product was purified by preparative HPLC (Method 9) to yield the title compound (54.8 mg, 55% yield).

LC-MS (Method 3): Rₜ=1.21 min; MS (ESIpos) m/z=530.2 [M+H]⁺.

¹H-NMR (400 MHz, DMSO-d₆): δ [ppm]=13.23 (br. s., 1H), 11.89-10.33 (m, 0.5H), 9.73 (s, 1H), 9.11-8.19 (m, 0.5H), 8.18-8.09 (m, 1H), 7.80 (s, 1H), 7.76 (d, 1H), 7.58-7.52 (m, 1H), 4.26-4.11 (m, 1H), 3.84-3.66 (m, 1H), 3.48-3.38 (m, 1H), 3.28 (s, 3H), 2.61-2.52 (m, 1H), 2.11-1.82 (m, 4H), 1.69-1.28 (m, 4H), 1.16 (d, 3H).

### Example 144

### N⁵-(trans-4-{[2-chloro-5-(trifluoromethyl)phenyl]carbamoyl}cyclohexyl)-N⁴-(2-methoxy-2-methylpropyl)-1H-imidazole-4,5-dicarboxamide

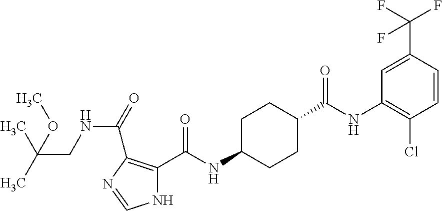 [SMILES: COC(C)(C)CNC(=O)c1nc[nH]c1C(=O)N[C@H]1CC[C@H](C(=O)Nc2cc(C(F)(F)F)ccc2Cl)CC1]

A mixture of phenyl 5-[(trans-4-{[2-chloro-5-(trifluoromethyl)phenyl]carbamoyl}cyclohexyl)carbamoyl]-1H-imidazole-4-carboxylate (123 mg, 0.230 mmol,) and 2-methoxy-2-methylpropan-1-amine (119 mg, 1.15 mmol, CAS No 89282-70-2) in tetrahydrofuran (0.51 ml) was stirred at 60° C. over night. For work-up the reaction mixture was concentrated and the crude product was stirred with methanol. The precipitate was collected by filtration and dried to yield the title compound (41.0 mg, 32% yield).

LC-MS (Method 6): Rₜ=1.25 min; MS (ESIpos) m/z=544.2 [M+H]⁺.

¹H-NMR (400 MHz, DMSO-d₆): δ [ppm]=13.01-11.91 (m, 1H), 11.23-10.98 (m, 1H), 9.79-9.67 (m, 1H), 8.48-8.35 (m, 0.5H), 8.19-8.12 (m, 1H), 8.11-8.00 (m, 0.5H), 7.83 (s, 1H), 7.79-7.72 (m, 1H), 7.60-7.50 (m, 1H), 3.88-3.55 (m, 1H), 3.37 (d, 2H), 3.19-3.12 (m, 3H), 2.63-2.54 (m, 1H), 2.14-1.79 (m, 4H), 1.66-1.45 (m, 3H), 1.43-1.21 (m, 1H), 1.14 (s, 6H).

### Example 145

### N⁵-(trans-4-{[2-chloro-5-(trifluoromethyl)phenyl]carbamoyl}cyclohexyl)-N⁴-(2-hydroxy-2-methylpropyl)-1H-imidazole-4,5-dicarboxamide

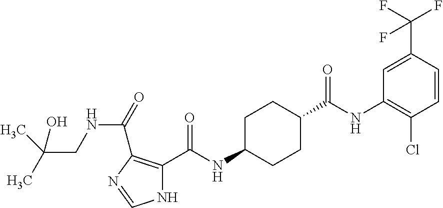 [SMILES: CC(C)(O)CNC(=O)c1nc[nH]c1C(=O)N[C@H]1CC[C@H](C(=O)Nc2cc(C(F)(F)F)ccc2Cl)CC1]

A mixture of phenyl 5-[(trans-4-{[2-chloro-5-(trifluoromethyl)phenyl]carbamoyl}cyclohexyl)carbamoyl]-1H-imidazole-4-carboxylate (123 mg, 0.230 mmol,) and 1-amino-2-methylpropan-2-ol (102 mg, 1.15 mmol, CAS No 2854-16-2) in tetrahydrofuran (0.51 ml) was stirred at 60° C. over night. For work-up the reaction mixture was concentrated and the crude product was purified by preparative HPLC (Method 9) to yield the title compound (47.0 mg, 39% yield).

LC-MS (Method 3): Rₜ=1.09 min; MS (ESIpos) m/z=530.2 [M+H]⁺.

¹H-NMR (400 MHz, DMSO-d₆): δ [ppm]=13.39-13.12 (m, 1H), 11.27-10.95 (m, 1H), 9.82-9.58 (m, 1H), 8.49-8.09 (m, 2H), 7.83 (s, 1H), 7.78-7.72 (m, 1H), 7.60-7.50 (m, 1H), 4.84-4.46 (m, 1H), 3.91-3.63 (m, 1H), 3.27 (d, 2H), 2.14-1.80 (m, 4H), 1.69-1.44 (m, 3H), 1.41-1.20 (m, 1H), 1.13 (s, 6H).

### Example 146

### N⁵-(trans-4-{[2-chloro-5-(trifluoromethyl)phenyl]carbamoyl}cyclohexyl)-N⁴-(tetrahydrofuran-2-ylmethyl)-1H-imidazole-4,5-dicarboxamide

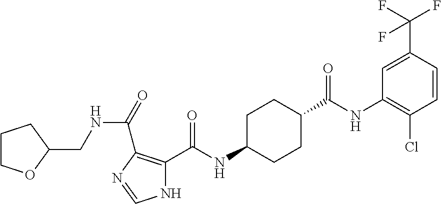 [SMILES: O=C(NCC1CCCO1)c1nc[nH]c1C(=O)N[C@H]1CC[C@H](C(=O)Nc2cc(C(F)(F)F)ccc2Cl)CC1]

A mixture of phenyl 5-[(trans-4-{[2-chloro-5-(trifluoromethyl)phenyl]carbamoyl}cyclohexyl)carbamoyl]-1H-imidazole-4-carboxylate (100 mg, 0.187 mmol,) and 1-(tetrahydrofuran-2-yl)methanamine (88 mg, 0.87 mmol, CAS No 4795-29-3) in tetrahydrofuran (5.0 ml) was stirred at room temperature over night. For work-up the reaction mixture was concentrated and the crude product was stirred with methanol. The precipitate was collected by filtration, and dried to yield the title compound (74.0 mg, 78% yield).

LC-MS (Method 6): Rₜ=1.20 min; MS (ESIpos) m/z=542.2 [M+H]⁺.

¹H-NMR (400 MHz, DMSO-d₆): δ [ppm]=13.34-13.01 (m, 1H), 11.55-10.77 (m, 1H), 9.73 (s, 1H), 8.75-8.23 (m, 1H), 8.16 (d, 1H), 7.83 (s, 1H), 7.76 (d, 1H), 7.60-7.50 (m, 1H), 4.07-3.91 (m, 1H), 3.87-3.69 (m, 2H), 3.64 (d, 1H), 3.37 (d, 2H), 2.66-2.55 (m, 1H), 2.14-1.70 (m, 7H), 1.69-1.17 (m, 5H).

### Example 147

### N⁵-{trans-4-[(5-chloropyrimidin-4-yl)carbamoyl]cyclohexyl}-N⁴-methyl-1H-imidazole-4,5-dicarboxamide

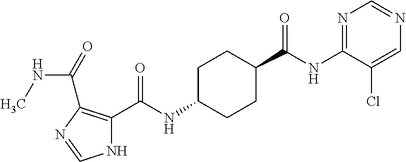 [SMILES: CNC(=O)c1nc[nH]c1C(=O)N[C@H]1CC[C@H](C(=O)Nc2ncncc2Cl)CC1]

Methanamine (320 μl, 2.0 M solution in tetrahydrofuran, 640 μmol) was added to a solution of phenyl 5-({trans-4-[(5-chloropyrimidin-4-yl)carbamoyl]cyclohexyl}carbamoyl)-1H-imidazole-4-carboxylate (60.0 mg, 128 μmol) in diochloromethane (3.0 ml) and the mixture was stirred at room temperature for 24 h. For work-up, the reaction mixture was concentrated and the residue was purified by preparative HPLC (Method 9) followed by recrystallization from methanol to give the title compound (9.50 mg, 91% purity, 17% yield).

LC-MS (Method 1): Rₜ=0.73 min; MS (ESIpos): m/z=406.3 [M+H]⁺

¹H-NMR (400 MHz, DMSO-d₆): δ [ppm]=13.48-12.63 (m, 1H), 11.56-10.25 (m, 1H), 8.92 (s, 1H), 8.85 (s, 1H), 8.78-8.25 (m, 1H), 7.82-7.77 (m, 1H), 3.83-3.58 (m, 1H), 2.85-2.77 (m, 3H), 2.60-2.54 (m, 1H), 2.19-1.82 (m, 4H), 1.64-1.19 (m, 4H).

### Example 148

### N-[(cis)-2-(2-chloro-4-fluorophenyl)-1-oxo-2-azaspiro[4.5]dec-8-yl]pyrazolo[1,5-a]pyrimidine-3-carboxamide

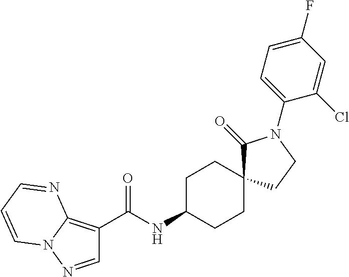 [SMILES: O=C(N[C@H]1CC[C@]2(CCN(c3ccc(F)cc3Cl)C2=O)CC1)c1cnn2cccnc12]

Was prepared in analogy to the synthesis of N-[(trans)-2-(2-chloro-4-fluorophenyl)-1-oxo-2-azaspiro[4.5]dec-8-yl]pyrazolo[1,5-a]pyrimidine-3-carboxamide from 8-amino-2-(2-chloro-4-fluorophenyl)-2-azaspiro[4.5]decan-1-one (isomer 2, intermediate I63) (131 mg, crude product). For work-up, the reaction mixture was concentrated under reduced pressure and the residue was purified by flash chromatography (Snap cartridge, dichloromethane/methanol gradient) followed by preparative HPLC (Method 8) to yield the title compound (38.0 mg, 99% purity).

LC-MS (Method 1): Rₜ=1.06 min; MS (ESIpos) m/z=442.3 [M+H]⁺.

¹H-NMR (400 MHz, DMSO-d₆): δ [ppm]=9.32 (dd, 1H), 8.81 (dd, 1H), 8.58 (s, 1H), 8.06 (br. d, 1H), 7.60 (dd, 1H), 7.53-7.46 (m, 1H), 7.37-7.23 (m, 2H), 4.15-4.00 (m, 1H), 3.63 (t, 2H), 2.09 (t, 2H), 2.03-1.86 (m, 4H), 1.85-1.74 (m, 2H), 1.61-1.51 (m, 2H).

### Example 149

### N-(trans-4-{[4-chloro-6-(trifluoromethyl)pyridin-3-yl]carbamoyl}cyclohexyl)pyrazolo[1,5-a]pyrimidine-3-carboxamide

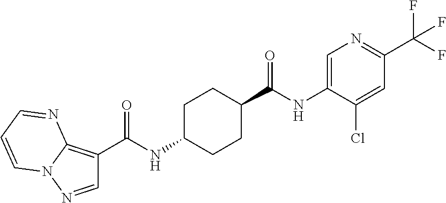 [SMILES: O=C(N[C@H]1CC[C@H](C(=O)Nc2cnc(C(F)(F)F)cc2Cl)CC1)c1cnn2cccnc12]

1-Chlor-1-dimethylamino-2-methyl-1-propen (139 mg, 1.04 mmol) was added to a solution of trans-4-[(pyrazolo[1,5-a]pyrimidin-3-ylcarbonyl)amino]cyclohexanecarboxylic acid (100 mg, 0.347 mmol) in dichloromethane (6.6 ml) and the mixture was stirred at room temperature for 30 min. Pyridine (140 μl, 1.7 mmol) and 4-chloro-6-(trifluoromethyl)pyridin-3-amine (136 mg, 0.694 mmol, CAS No. 1196153-86-2,) were added and the mixture was stirred over night at room temperature followed by 4 h at 40° C. For work-up, the reaction mixture was concentrated and the residue was purified by preparative HPLC (Method 8) to give the title compound (65.6 mg, 40% yield).

LC-MS (Method 4): Rₜ=1.05 min; MS (ESIpos) m/z=467.2 [M+H]⁺.

¹H-NMR (400 MHz, DMSO-d₆): δ [ppm]=10.04 (br. s., 1H), 9.32 (dd, 1H), 9.05 (s, 1H), 8.83 (dd, 1H), 8.58 (s, 1H), 8.20 (s, 1H), 7.80 (d, 1H), 7.30-7.24 (m, 1H), 3.91-3.77 (m, 1H), 2.65-2.56 (m, 1H), 2.14-1.93 (m, 4H), 1.68-1.53 (m, 2H), 1.47-1.33 (m, 2H).

### Example 150

### N-{trans-4-[(2-chloro-4-fluoro-5-methylphenyl)carbamoyl]cyclohexyl}pyrazolo[1,5-a]pyrimidine-3-carboxamide

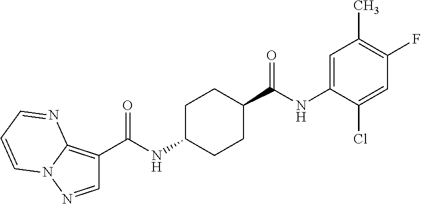 [SMILES: Cc1cc(NC(=O)[C@H]2CC[C@H](NC(=O)c3cnn4cccnc34)CC2)c(Cl)cc1F]

1-Chlor-1-dimethylamino-2-methyl-1-propen (139 mg, 1.04 mmol) was added to a solution of trans-4-[(pyrazolo[1,5-a]pyrimidin-3-ylcarbonyl)amino]cyclohexanecarboxylic acid (100 mg, 0.347 mmol) in dichloromethane (6.6 ml) and the mixture was stirred at room temperature for 30 min. Pyridine (140 μl, 1.7 mmol) and 2-chloro-4-fluoro-5-methylaniline (111 mg, 0.694 mmol, CAS No. 124185-35-9) were added and the mixture was stirred over night at room temperature followed by 3 h at 40° C. For work-up, the reaction mixture was concentrated and the residue was stirred with dimethyl sulfoxide, the precipitated was collected by filtration, washed and dried to yield the title compound (119 mg, 78% yield).

LC-MS (Method 2): Rₜ=1.09 min; MS (ESIpos) m/z=430.2 [M+H]⁺.

¹H-NMR (400 MHz, DMSO-d₆): δ [ppm]=9.46 (br. s, 1H), 9.32 (dd, 1H), 8.82 (dd, 1H), 8.58 (s, 1H), 7.79 (d, 1H), 7.48 (d, 1H), 7.41 (d, 1H), 7.31-7.24 (m, 1H), 3.92-3.74 (m, 1H), 2.48-2.39 (m, 1H), 2.21 (d, 3H), 2.10-2.00 (m, 2H), 1.99-1.89 (m, 2H), 1.67-1.50 (m, 2H), 1.45-1.30 (m, 2H).

### Example 151

### N-{trans-4-[(2-chloro-4-fluoro-6-methylphenyl)carbamoyl]cyclohexyl}pyrazolo[1,5-a]pyrimidine-3-carboxamide

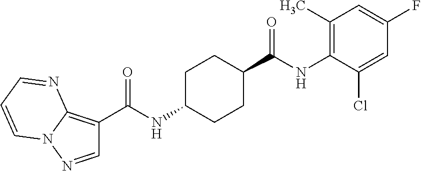 [SMILES: Cc1cc(F)cc(Cl)c1NC(=O)[C@H]1CC[C@H](NC(=O)c2cnn3cccnc23)CC1]

1-Chlor-1-dimethylamino-2-methyl-1-propen (139 mg, 1.04 mmol) was added to a solution of trans-4-[(pyrazolo[1,5-a]pyrimidin-3-ylcarbonyl)amino]cyclohexanecarboxylic acid (100 mg, 0.347 mmol) in dichloromethane (6.6 ml) and the mixture was stirred at room temperature for 30 min. Pyridine (140 μl, 1.7 mmol) and 2-chloro-4-fluoro-6-methylaniline (111 mg, 0.694 mmol, CAS No. 332903-47-6) were added and the mixture was stirred over night at room temperature followed by 4 h at 40° C. For work-up, the reaction mixture was concentrated and the residue was purified by preparative HPLC (Method 8) to yield the title compound (65.6 mg, 40% yield).

LC-MS (Method 1): Rₜ=0.97 min; MS (ESIpos) m/z=430.2 [M+H]⁺.

¹H-NMR (400 MHz, DMSO-d₆): δ [ppm]=9.44 (s, 1H), 9.32 (dd, 1H), 8.83 (dd, 1H), 8.58 (s, 1H), 7.79 (d, 1H), 7.34 (dd, 1H), 7.30-7.25 (m, 1H), 7.17 (dd, 1H), 3.91-3.74 (m, 1H), 2.47-2.36 (m, 1H), 2.17 (s, 3H), 2.13-1.92 (m, 4H), 1.69-1.54 (m, 2H), 1.46-1.33 (m, 2H).

### Example 152

### N-{trans-4-[(2-chloro-6-cyano-4-fluorophenyl)carbamoyl]cyclohexyl}pyrazolo[1,5-a]pyrimidine-3-carboxamide

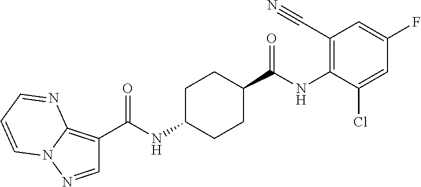 [SMILES: N#Cc1cc(F)cc(Cl)c1NC(=O)[C@H]1CC[C@H](NC(=O)c2cnn3cccnc23)CC1]

Prepared in analogy to the synthesis of N-(trans-4-{[4-chloro-6-(trifluoromethyl)pyridin-3-yl]carbamoyl}cyclohexyl)pyrazolo[1,5-a]pyrimidine-3-carboxamide using 2-amino-3-chloro-5-fluorobenzonitrile (82.8 mg, 486 μmol, CAS No: 1263277-C₆-0) as starting material. For work-up, the reaction mixture was concentrated and the residue was purified by preparative HPLC (Method 9) to give the title compound (2.8 mg).

LC-MS (Method 1): Rₜ=0.92 min; MS (ESIpos): m/z=441.1 [M+H]⁺

¹H-NMR (400 MHz, DMSO-d₆): δ [ppm]=10.15 (s, 1H), 9.32 (dd, 1H), 8.83 (dd, 1H), 8.60-8.55 (m, 1H), 8.06-7.95 (m, 2H), 7.80 (d, 1H), 7.27 (dd, 1H), 3.90-3.75 (m, 1H), 2.12-1.94 (m, 4H), 1.69-1.54 (m, 2H), 1.51-1.32 (m, 2H).

### Example 153

### N-{trans-4-[(5-chloro-2-methyl pyridin-4-yl)carbamoyl]cyclohexyl}pyrazolo[1,5-a]pyrimidine-3-carboxamide

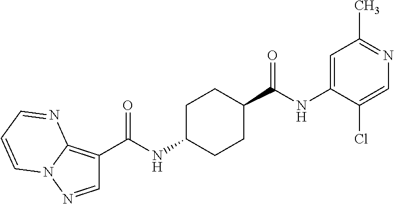 [SMILES: Cc1cc(NC(=O)[C@H]2CC[C@H](NC(=O)c3cnn4cccnc34)CC2)c(Cl)cn1]

Prepared in analogy to the synthesis of N-(trans-4-{[4-chloro-6-(trifluoromethyl)pyridin-3-yl]carbamoyl}cyclohexyl)pyrazolo[1,5-a]pyrimidine-3-carboxamide using 5-chloro-2-methylpyridin-4-amine dihydrochloride (67.3 mg, 312 μmol) as starting material and 3 days reaction time. For work-up, water was added and the mixture was extracted with dichloromethane and the organic phase was washed with saturated sodium bicarbonate solution and brine. The organic phase was dried by filtration through a silicone filter and concentrated. The residue was purified by preparative HPLC to give the title compound (2.3 mg).

LC-MS (Method 1): Rₜ=0.76 min; MS (ESIpos): m/z=413.3 [M+H]⁺

¹H-NMR (400 MHz, DMSO-d6) delta [ppm]: 1.209 (0.95), 1.333 (0.95), 1.339 (0.95), 1.342 (0.95), 1.363 (1.06), 1.367 (1.11), 1.372 (1.11), 1.395 (0.53), 1.401 (0.48), 1.405 (0.48), 1.494 (0.42), 1.502 (0.48), 1.527 (1.11), 1.529 (1.11), 1.536 (1.01), 1.557 (1.01), 1.564 (1.01), 1.566 (1.06), 1.590 (0.42), 1.880 (0.95), 1.905 (1.11), 1.912 (1.27), 1.943 (1.06), 1.949 (0.95), 2.016 (1.01), 2.020 (1.11), 2.026 (1.17), 2.049 (1.11), 2.052 (1.06), 2.059 (0.95), 2.295 (0.64), 2.299 (1.38), 2.304 (1.96), 2.309 (1.43), 2.314 (0.64), 2.403 (16.00), 2.612 (0.48), 2.637 (1.06), 2.642 (2.28), 2.646 (2.44), 2.651 (1.96), 2.656 (0.85), 2.671 (0.42), 3.346 (0.64), 3.777 (0.48), 3.787 (0.58), 3.797 (0.48), 3.806 (0.58), 3.816 (0.42), 7.236 (1.91), 7.247 (1.80), 7.254 (1.85), 7.264 (1.85), 7.766 (1.54), 7.786 (1.54), 7.886 (4.98), 8.415 (5.83), 8.555 (6.94), 8.795 (2.07), 8.800 (2.38), 8.806 (2.23), 8.810 (1.96), 9.284 (2.23), 9.288 (2.17), 9.302 (2.23), 9.306 (1.85), 9.553 (2.17).

### Example 154

### N-{trans-4-[(2-chloro-4-fluorophenyl)(ethyl)carbamoyl]cyclohexyl}pyrazolo[1,5-a]pyrimidine-3-carboxamide

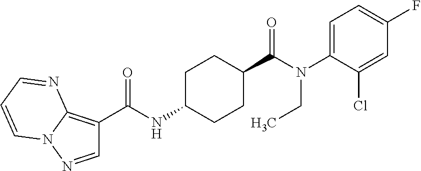 [SMILES: CCN(c1ccc(F)cc1Cl)C(=O)[C@H]1CC[C@H](NC(=O)c2cnn3cccnc23)CC1]

Prepared in analogy to the synthesis of N-(trans-4-{[4-chloro-6-(trifluoromethyl)pyridin-3-yl]carbamoyl}cyclohexyl)pyrazolo[1,5-a]pyrimidine-3-carboxamide using 2-chloro-N-ethyl-4-fluoroaniline (96.4 mg, 555 μmol) as starting material. For workup the reaction mixture was concentrated and the residue was purified by preparative HPLC (Method 8) to give the title compound (62.9 mg).

LC-MS (Method 1): Rₜ=1.13 min; MS (ESIpos): m/z=444.3 [M+H]⁺

¹H-NMR (400 MHz, DMSO-d₆): δ [ppm]=9.35-9.25 (m, 1H), 8.84-9.68 (m, 1H), 8.60-8.50 (m, 1H), 7.84-7.50 (m, 3H), 7.37 (td, 1H), 7.30-7.17 (m, 1H), 3.88-3.65 (m, 2H), 2.11-1.78 (m, 3H), 1.75-1.35 (m, 4H), 1.17-0.92 (m, 5H).

### Example 155

### N-{trans-4-[(2-chloro-4-fluorophenyl)(methyl)carbamoyl]cyclohexyl}pyrazolo[1,5-a]pyrimidine-3-carboxamide

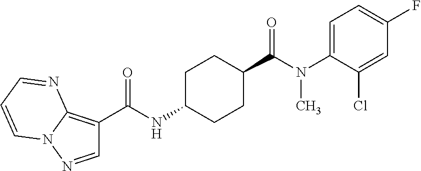 [SMILES: CN(c1ccc(F)cc1Cl)C(=O)[C@H]1CC[C@H](NC(=O)c2cnn3cccnc23)CC1]

1-Chlor-1-dimethylamino-2-methyl-1-propen (96 μl, 730 μmol) was added to a solution of trans-4-[(pyrazolo[1,5-a]pyrimidin-3-ylcarbonyl)amino]cyclohexanecarboxylic acid (70.0 mg, 243 μmol) in dichloromethane (4.6 ml) and the mixture was stirred at room temperature for 30 min. Pyridine (98 μl, 1.2 mmol) and 2-chloro-4-fluoro-N-methylaniline (77.5 mg, 486 μmol) were added and the mixture was stirred for 3 days at room temperature. Water was added and the mixture was extracted with dichloromethane. The combined organic phases were washed with saturated sodium bicarbonate solution and water and the organic phases were dried and concentrated. The residue was purified by preparative HPLC (Method 8) to yield the title compound (68.0 mg, 92% purity, 60% yield).

LC-MS (Method 1): Rₜ=1.06 min; MS (ESIpos): m/z=430.3 [M+H]⁺

¹H-NMR (400 MHz, DMSO-d₆): δ [ppm]=9.35-9.23 (m, 1H), 8.83-8.69 (m, 1H), 8.62-8.48 (m, 1H), 7.84-7.52 (m, 3H), 7.37 (td, 1H), 7.30-7.19 (m, 1H), 3.80-3.67 (m, 1H), 3.06 (s, 3H), 2.11-1.81 (m, 3H), 1.77-1.35 (m, 4H), 1.18-0.94 (m, 2H).

### Example 156

### N-{trans-4-[methyl(phenyl)carbamoyl]cyclohexyl}pyrazolo[1,5-a]pyrimidine-3-carboxamide

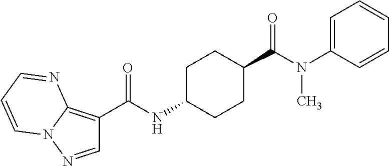 [SMILES: CN(c1ccccc1)C(=O)[C@H]1CC[C@H](NC(=O)c2cnn3cccnc23)CC1]

Prepared in analogy to the synthesis of N-(trans-4-{[4-chloro-6-(trifluoromethyl)pyridin-3-yl]carbamoyl}cyclohexyl)pyrazolo[1,5-a]pyrimidine-3-carboxamide N-methylaniline (66.9 mg, 624 μmol) as starting material. For workup the reaction mixture was concentrated and the residue was purified by preparative HPLC (Method 8) to give the title compound (99.0 mg). LC-MS (Method 1): Rₜ=0.97 min; MS (ESIpos): m/z=378.4 [M+H]⁺

¹H-NMR (400 MHz, DMSO-d₆): δ [ppm]=9.28 (dd, 1H), 8.73 (dd, 1H), 8.52 (s, 1H), 7.67 (d, 1H), 7.53-7.44 (m, 2H), 7.43-7.31 (m, 3H), 7.23 (dd, 1H), 3.86-3.63 (m, 1H), 3.14 (br. s., 3H), 2.22-2.04 (m, 1H), 1.98-1.81 (m, 2H), 1.77-1.62 (m, 2H), 1.60-1.43 (m, 2H), 1.12-0.87 (m, 2H).

### Example 157

### N⁵-{trans-4-[(2-chloro-4-fluorophenyl)(2-hydroxyethyl)carbamoyl]cyclohexyl}-N⁴-methyl-1H-imidazole-4,5-dicarboxamide

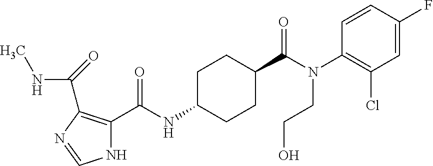 [SMILES: CNC(=O)c1nc[nH]c1C(=O)N[C@H]1CC[C@H](C(=O)N(CCO)c2ccc(F)cc2Cl)CC1]

To a suspension of 207 mg (0.39 mmol) phenyl 5-({trans-4-[(2-chloro-4-fluorophenyl)(2-hydroxyethyl)carbamoyl]cyclohexyl}carbamoyl)-1H-imidazole-4-carboxylate in 5 ml tetrahydrofuran were added 319 μl (2 M in tetrahydrofuran, 0.78 mmol) methanamine. The suspension was stirred for 3 hours at room temperature. The mixture was diluted with water and the aqueous phase was extracted with ethyl acetate. The organic phase was dried over sodium sulphate, and the solvent was removed under reduced pressure. The crude product was purified by HPLC to give 58 mg of the title compound as a solid material.

LC-MS (Method 6): Rₜ=1.15 min; MS (ESIpos) m/z=466.2 [M+H]⁺.

¹H-NMR (400 MHz, DMSO-d₆), δ [ppm]=13.20 (br. s., 1H), 11.24 (br. s., 1H), 8.61 (br. s., 1H), 7.80 (s, 1H), 7.26 (dd, 1H), 7.05 (td, 1H), 6.81 (dd, 1H), 5.31 (t, 1H), 4.18 (t, 2H), 3.70 (br. s., 1H), 3.40 (q, 2H), 2.81 (d, 3H), 1.94 (d, 4H), 1.58-1.14 (m, 4H).

### Example 158

### N⁵-{trans-4-[(2-chlorophenyl)(ethyl)carbamoyl]cyclohexyl}-N⁴-methyl-1H-imidazole-4,5-dicarboxamide

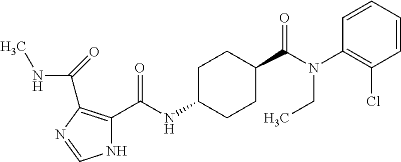 [SMILES: CCN(c1ccccc1Cl)C(=O)[C@H]1CC[C@H](NC(=O)c2[nH]cnc2C(=O)NC)CC1]

To a suspension of 1.38 g (2.79 mmol) phenyl 5-({trans-4-[(2-chlorophenyl)(ethyl)carbamoyl]cyclohexyl}carbamoyl)-1H-imidazole-4-carboxylate in tetrahydrofuran were added 7 ml (2 M in tetrahydrofuran, 13.9 mmol) methanamine. The suspension was stirred for 3 days at room temperature. The reaction mixture was concentrated in vacuo to obtain the crude product, which was purified by flash column chromatography (dichloromethane/ethyl acetate-gradient) to give 40 mg (90% purity) of the title compound as a solid material.

LC-MS (Method 1): Rₜ=1.09 min; MS (ESIpos) m/z=432.5 [M+H]⁺.

¹H-NMR (400 MHz, DMSO-d₆), δ [ppm]=13.26-12.99 (m, 1H), 11.17-10.87 (m, 1H), 8.60-8.50 (m, 0.7H), 8.26-8.14 (m, 0.3H), 7.76 (d, 0.6), 7.75-7.73 (m, 0.4H), 7.70-7.66 (m, 1H), 7.55-7.27 (m, 3H), 3.89-3.79 (m, 1H), 3.73-3.53 (m, 1H), 3.36-3.28 (m, 1H), 2.81 (d, 1.2H), 2.72 (d, 1.8H), 1.94-1.33 (m, 7H), 1.25-1.05 (m, 1H), 1.02-0.96 (m, 3H), 0.96-0.76 (m, 1H).

### Example 159

### N⁵-[(trans)-2-(2-chloro-4-fluorophenyl)-1-oxo-2-azaspiro[5.5]undec-9-yl]-N⁴-methyl-1H-imidazole-4,5-dicarboxamide)

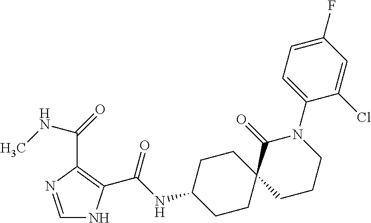 [SMILES: CNC(=O)c1nc[nH]c1C(=O)N[C@H]1CC[C@@]2(CCCN(c3ccc(F)cc3Cl)C2=O)CC1]

To the crude reaction mixture of phenyl 5-{[2-(2-chloro-4-fluorophenyl)-1-oxo-2-azaspiro[5.5]undec-9-yl]carbamoyl}-1H-imidazole-4-carboxylate (isomer 1) (0.59 mmol) were added 1.5 ml (2 M in tetrahydrofuran, 3.00 mmol) methanamine. The suspension was stirred for 1 hour at room temperature. The reaction mixture was concentrated in vacuo to obtain the crude product, which was purified by flash column chromatography (dichloromethane/ethanol-gradient) and HPLC chromatography to give 15 mg of the title compound as a solid material.

LC-MS (Method 2): Rₜ=1.08 min; MS (ESIpos) m/z=462.1 [M+H]⁺.

¹H-NMR (400 MHz, DMSO-d₆), δ [ppm]=12.89 (br. s., 1H), 11.21-10.70 (m, 1H), 8.40-8.08 (m, 1H), 7.76-7.63 (m, 1H), 7.46 (dd, 1H), 7.42-7.33 (m, 1H), 7.23 (td, 1H), 3.87-3.70 (m, 1H), 3.49 (d, 2H), 2.86 (d, 3H), 2.07-1.85 (m, 7H), 1.84-1.37 (m, 5H).

### Example 160

### N⁵-[(trans)-2-(2-chloro-4-fluorophenyl)-1-oxo-2-azaspiro[4.5]dec-8-yl]-N⁴-[2-(4-methylpiperidin-1-yl)ethyl]-1H-imidazole-4,5-dicarboxamide

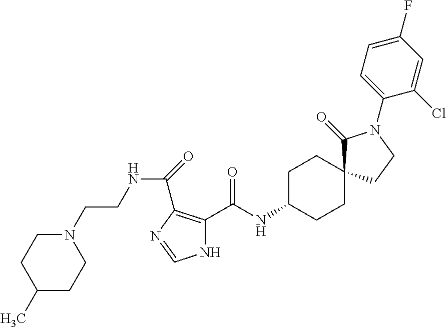 [SMILES: CC1CCN(CCNC(=O)c2nc[nH]c2C(=O)N[C@H]2CC[C@@]3(CCN(c4ccc(F)cc4Cl)C3=O)CC2)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added (4-methylpiperidin-1-yl)ethanamine (55.7 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The acetonitrile was removed under reduced pressure and diethyl ether was added to the aqueous solution. The resulting precipitate was filtered off to give after drying under vacuum 8 mg of the title compound as a solid material.

LC-MS (Method 6): Rₜ=0.79 min; MS (ESIpos) m/z=559.3 [M+H]⁺.

¹H-NMR (400 MHz, DMSO-d₆), δ [ppm]=13.19 (br. s., 1H), 11.32-10.89 (m, 1H), 8.49-8.20 (m, 1H), 7.81 (s, 1H), 7.60-7.57 (m, 1H), 7.53-7.38 (m, 1H), 7.38-7.23 (m, 1H), 3.76 (br. s., 1H), 3.63 (t, 2H), 3.40 (d, 2H), 2.86 (d, 2H), 2.15 (br. s., 2H), 2.01-1.91 (m, 3H), 1.82 (br. s., 1H), 1.76-1.51 (m, 7H), 1.48-1.03 (m, 5H), 0.89 (d, 3H).

### Example 161

### N⁵-[(trans)-2-(2-chloro-4-fluorophenyl)-1-oxo-2-azaspiro[4.5]dec-8-yl]-N⁴-[2-(piperidin-1-yl)ethyl]-1H-imidazole-4,5-dicarboxamide

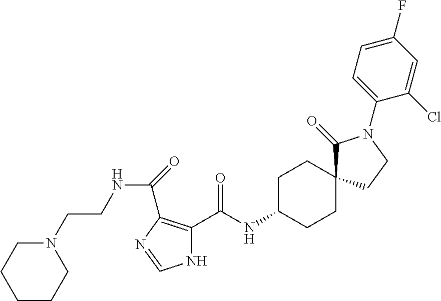 [SMILES: O=C(NCCN1CCCCC1)c1nc[nH]c1C(=O)N[C@H]1CC[C@@]2(CCN(c3ccc(F)cc3Cl)C2=O)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 2-(piperidin-1-yl)ethanamine (50 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The acetonitrile was removed under reduced pressure and diethyl ether was added to the aqueous solution. The resulting precipitate was filtered off to give after drying under vacuum 18 mg of the title compound as a solid material.

LC-MS (Method 1): Rₜ=0.85 min; MS (ESIpos) m/z=545.4 [M+H]⁺.

¹H-NMR (400 MHz, DMSO-d₆), δ [ppm]=13.23 (br. s., 1H), 11.30-10.96 (m, 1H), 8.60-8.31 (m, 1H), 7.82 (s, 1H), 7.60 (dd, 1H), 7.49 (dd, 1H), 7.32 (td, 1H), 3.75 (br. s., 1H), 3.63 (t, 2H), 3.47-3.36 (m, 2H), 2.47-2.35 (m, 5H), 2.14 (t, 2H), 2.07-1.59 (m, 7H), 1.55-1.29 (m, 7H).

### Example 162

### N⁵-[(trans)-2-(2-chloro-4-fluorophenyl)-1-oxo-2-azaspiro[4.5]dec-8-yl]-N⁴-isopropyl-1H-imidazole-4,5-dicarboxamide

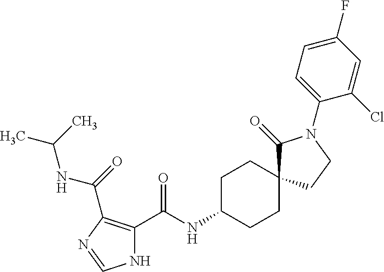 [SMILES: CC(C)NC(=O)c1nc[nH]c1C(=O)N[C@H]1CC[C@@]2(CCN(c3ccc(F)cc3Cl)C2=O)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added propan-2-amine (23.1 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and the resulting precipitate was filtered off to give after drying under vacuum 30 mg of the title compound as a solid material.

LC-MS (Method 1): Rₜ=1.15 min; MS (ESIpos) m/z=476.2 [M+H]⁺.

¹H-NMR (400 MHz, DMSO-d₆), δ [ppm]=13.22 (br. s., 1H), 11.45-10.63 (m, 1H), 8.51-8.14 (m, 1H), 7.82 (s, 1H), 7.60 (dd, 1H), 7.48 (dd, 1H), 7.32 (td, 1H), 4.22-3.97 (m, 1H), 3.90-3.69 (m, 1H), 3.63 (t, 2H), 2.14 (br. s., 2H), 1.98 (br. s., 1H), 1.84-1.58 (m, 6H), 1.51-1.34 (m, 1H), 1.19 (d, 6H).

### Example 163

### N⁵-[(trans)-2-(2-chloro-4-fluorophenyl)-1-oxo-2-azaspiro[4.5]dec-8-yl]-N⁴-(2-methoxyethyl)-1H-imidazole-4,5-dicarboxamide

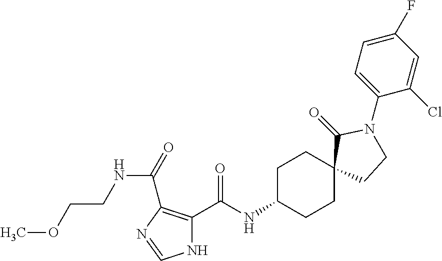 [SMILES: COCCNC(=O)c1nc[nH]c1C(=O)N[C@H]1CC[C@@]2(CCN(c3ccc(F)cc3Cl)C2=O)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 2-methoxyethanamine (29.4 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and diethyl ether was added to the aqueous solution. The resulting precipitate was filtered off to give after drying under vacuum 45 mg of the title compound as a solid material.

LC-MS (Method 1): Rₜ=1.05 min; MS (ESIpos) m/z=492.1 [M+H]⁺.

¹H-NMR (400 MHz, DMSO-d₆), δ [ppm]=13.25 (br. s., 1H), 11.31-10.94 (m, 1H), 8.61-8.33 (m, 1H), 7.83 (s, 1H), 7.60 (dd, 1H), 7.48 (dd, 1H), 7.32 (td, 1H), 3.90-3.69 (m, 1H), 3.63 (t, 2H), 3.52-3.42 (m, 4H), 3.29 (s, 3H), 2.20-2.07 (m, 2H), 2.04-1.92 (m, 1H), 1.87-1.57 (m, 6H), 1.42 (d, 1H).

### Example 164

### N⁵-[(trans)-2-(2-chloro-4-fluorophenyl)-1-oxo-2-azaspiro[4.5]dec-8-yl]-N⁴-(1-methoxypropan-2-yl)-1H-imidazole-4,5-dicarboxamide

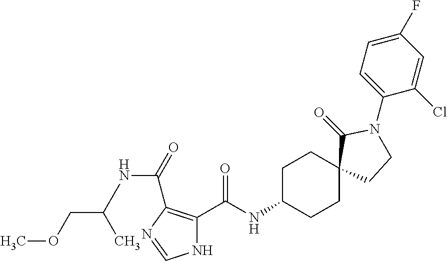 [SMILES: COCC(C)NC(=O)c1nc[nH]c1C(=O)N[C@H]1CC[C@@]2(CCN(c3ccc(F)cc3Cl)C2=O)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added (rac)-1-methoxypropan-2-amine (34.9 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and the resulting precipitate was filtered off to give after drying under vacuum 30 mg of the title compound as a solid material.

LC-MS (Method 6): Rₜ=1.08 min; MS (ESIpos) m/z=506.2 [M+H]⁺.

¹H-NMR (400 MHz, DMSO-d₆), δ [ppm]=13.21 (br. s., 1H), 11.12 (br. s., 1H), 8.49-8.10 (m, 1H), 7.81 (s, 1H), 7.59 (dd, 1H), 7.48 (dd, 1H), 7.31 (td, 1H), 4.28-4.06 (m, 1H), 3.94-3.71 (m, 1H), 3.63 (t, 2H), 3.49-3.33 (m, 2H), 3.29 (s, 3H), 2.15 (t, 2H), 2.00 (br. s., 1H), 1.80 (br. s., 6H), 1.43 (br. s., 1H), 1.17 (d, 3H).

### Example 165

### N⁵-[(trans)-2-(2-chloro-4-fluorophenyl)-1-oxo-2-azaspiro[4.5]dec-8-yl]-N⁴-cyclopropyl-1H-imidazole-4,5-dicarboxamide

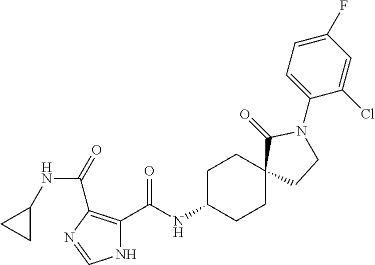 [SMILES: O=C(NC1CC1)c1nc[nH]c1C(=O)N[C@H]1CC[C@@]2(CCN(c3ccc(F)cc3Cl)C2=O)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added cyclopropanamine (22.3 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and the resulting precipitate was filtered off to give after drying under vacuum 40 mg of the title compound as a solid material.

LC-MS (Method 6): Rₜ=1.05 min; MS (ESIpos) m/z=474.2 [M+H]⁺.

¹H-NMR (400 MHz, DMSO-d₆), δ [ppm]=13.22 (br. s., 1H), 11.51-11.04 (m, 1H), 8.78-8.28 (m, 1H), 7.81 (br. s., 1H), 7.59 (dd, 1H), 7.48 (dd, 1H), 7.31 (td, 1H), 3.78 (br. s., 1H), 3.63 (t, 2H), 2.87 (br. s., 1H), 2.15 (br. s., 2H), 2.00 (br. s., 1H), 1.89-1.57 (m, 6H), 1.43 (br. s., 1H), 0.91-0.30 (m, 4H).

### Example 166

### N-[(trans)-2-(2-chloro-4-fluorophenyl)-1-oxo-2-azaspiro[4.5]dec-8-yl]-4-[(3-fluoroazetidin-1-yl)carbonyl]-1H-imidazole-5-carboxamide

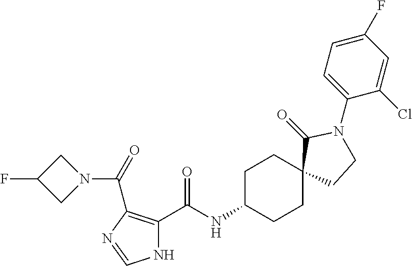 [SMILES: O=C(N[C@H]1CC[C@@]2(CCN(c3ccc(F)cc3Cl)C2=O)CC1)c1[nH]cnc1C(=O)N1CC(F)C1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 3-fluoroazetidinhydrochlorid (43.7 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and the resulting precipitate was filtered off to give after drying under vacuum 36 mg of the title compound as a solid material.

LC-MS (Method 6): Rₜ=1.03 min; MS (ESIpos) m/z=492.2 [M+H]⁺.

¹H-NMR (400 MHz, DMSO-d₆), δ [ppm]=13.28 (br. s., 1H), 11.07 (d, 1H), 7.83 (s, 1H), 7.59 (dd, 1H), 7.48 (dd, 1H), 7.31 (td, 1H), 5.67-5.22 (m, 1H), 5.06-4.84 (m, 1H), 4.74-4.54 (m, 1H), 4.51-4.28 (m, 1H), 4.21-4.01 (m, 1H), 3.80-3.68 (m, 1H), 3.63 (t, 2H), 2.14 (t, 2H), 2.05-1.92 (m, 2H), 1.77-1.59 (m, 4H), 1.48-1.32 (m, 2H).

### Example 167

### N-[(trans)-2-(2-chloro-4-fluorophenyl)-1-oxo-2-azaspiro[4.5]dec-8-yl]-4-{[4-(2,2,2-trifluoroethyl)piperazin-1-yl]carbonyl}-1H-imidazole-5-carboxamide

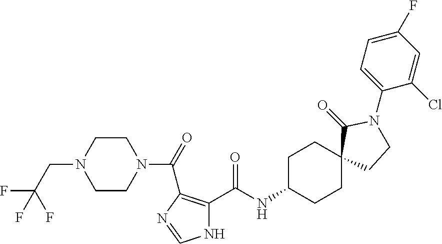 [SMILES: O=C(N[C@H]1CC[C@@]2(CCN(c3ccc(F)cc3Cl)C2=O)CC1)c1[nH]cnc1C(=O)N1CCN(CC(F)(F)F)CC1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 1-(trifluoromethyl)piperazine (65.8 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and the resulting precipitate was filtered off to give after drying under vacuum 25 mg of the title compound as a solid material.

LC-MS (Method 1): Rₜ=1.15 min; MS (ESIpos) m/z=585.2 [M+H]⁺.

¹H-NMR (400 MHz, DMSO-d₆), δ [ppm]=13.26-12.92 (m, 1H), 9.94 (d, 1H), 7.81 (s, 1H), 7.60 (dd, 1H), 7.48 (dd, 1H), 7.31 (td, 1H), 4.00 (br. s., 1H), 3.77-3.66 (m, 2H), 3.62 (t, 3H), 3.25 (q, 2H), 2.78-2.59 (m, 4H), 2.13 (t, 2H), 1.98 (d, 1H), 1.89-1.20 (m, 7H).

### Example 168

### N-[2-(2-chloro-4,5-difluorophenyl)-1-oxo-2-azaspiro[4.5]dec-8-yl]pyrazolo[1,5-a]pyrimidine-3-carboxamide (isomer 1)

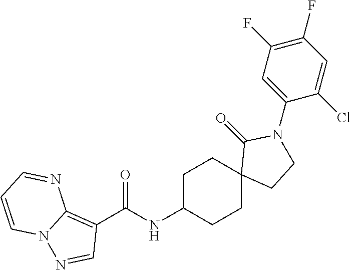 [SMILES: O=C(NC1CCC2(CC1)CCN(c1cc(F)c(F)cc1Cl)C2=O)c1cnn2cccnc12]

(Benzotriazol-1-yloxy)tripyrrolidinophosphonium hexafluorophosphate (296 mg, 570 μmol) was added to a mixture of 8-amino-2-(2-chloro-4,5-difluorophenyl)-2-azaspiro[4.5]decan-1-one (isomer 1) (326 mg), pyrazolo[1,5-a]pyrimidine-3-carboxylic acid (106 mg, 647 μmol, CAS No. 25940-35-6), and N-ethyl-N-isopropylpropan-2-amine (450 μl, 2.6 mmol) in N,N-dimethylformamide (5.8 ml) and the mixture was stirred for 12 h at room temperature. For work-up, the reaction mixture was concentrated under reduced pressure and the residue was purified by flash chromatography (ethyl acetate/ethanol gradient 0%->10% ethanol). DMSO was added to the obtained product and the precipitate thus formed was collected by filtration, washed with methanol and dried to give the title compound (38.5 mg).

LC-MS (Method 1) Rₜ=1.07 min; MS (ESIpos): m/z=460.3 [M+H]⁺

¹H-NMR (400 MHz, DMSO-d₆): δ [ppm]=9.32 (dd, 1H), 8.83 (dd, 1H), 8.58 (s, 1H), 7.93-7.78 (m, 2H), 7.72 (dd, 1H), 7.28 (dd, 1H), 3.92-3.77 (m, 1H), 3.64 (t, 2H), 2.16 (t, 2H), 2.05-1.91 (m, 2H), 1.73-1.63 (m, 4H), 1.58-1.39 (m, 2H).

### Example 169

### N-{trans-4-[(4-chloropyridin-3-yl)carbamoyl]cyclohexyl}-5,7-dimethylpyrazolo[1,5-a]pyrimidine-3-carboxamide

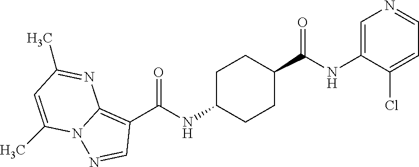 [SMILES: Cc1cc(C)n2ncc(C(=O)N[C@H]3CC[C@H](C(=O)Nc4cnccc4Cl)CC3)c2n1]

Was prepared in analogy to the synthesis of N-{trans-4-[(3-chloropyridin-4-yl)carbamoyl]cyclohexyl}-5,7-dimethylpyrazolo[1,5-a]pyrimidine-3-carboxamide using 5,7-dimethylpyrazolo[1,5-a]pyrimidine-3-carboxylic acid (58.6 mg, 306 μmol) and trans-4-amino-N-(4-chloropyridin-3-yl)cyclohexanecarboxamide hydrochloric acid salt (80.0 mg, 276 μmol) as starting materials to give the title compound (95.0 mg, 71% yield).

LC-MS (Method 1): Rₜ=0.92 min; MS (ESIpos): m/z=427.3 [M+H]⁺

1H-NMR (400 MHz, DMSO-d6) delta [ppm]: 1.358 (0.81), 1.364 (0.81), 1.368 (0.81), 1.388 (0.81), 1.394 (0.92), 1.397 (0.92), 1.420 (0.46), 1.580 (0.92), 1.586 (0.92), 1.589 (0.92), 1.610 (0.81), 1.617 (0.92), 1.619 (0.92), 1.957 (0.92), 1.964 (1.04), 1.966 (1.04), 1.993 (0.92), 1.997 (0.92), 2.002 (0.81), 2.069 (0.92), 2.078 (1.04), 2.101 (1.04), 2.104 (0.92), 2.111 (0.92), 2.327 (0.46), 2.523 (3.11), 2.533 (1.61), 2.543 (0.92), 2.562 (0.46), 2.623 (16.00), 2.635 (0.46), 2.669 (0.58), 2.726 (1.38), 2.733 (9.21), 2.735 (9.78), 2.886 (0.92), 3.332 (0.46), 3.340 (0.69), 3.346 (0.81), 3.351 (0.69), 3.356 (1.38), 3.443 (1.27), 3.449 (1.04), 3.454 (0.69), 3.461 (0.58), 3.466 (0.58), 3.789 (0.46), 3.798 (0.46), 3.808 (0.46), 7.119 (3.11), 7.121 (3.22), 7.606 (2.99), 7.620 (3.22), 7.991 (1.38), 8.011 (1.38), 8.338 (3.57), 8.351 (3.57), 8.494 (7.37), 8.731 (5.64), 9.752 (1.96).

### Example 170

### N⁵-(trans-4-{[2-chloro-5-(2-hydroxypropan-2-yl)phenyl]carbamoyl}cyclohexyl)-N⁴-methyl-1H-imidazole-4,5-dicarboxamide

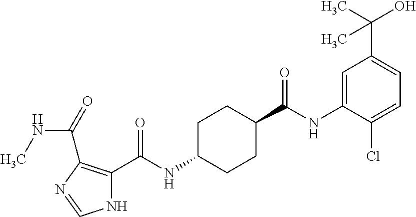 [SMILES: CNC(=O)c1nc[nH]c1C(=O)N[C@H]1CC[C@H](C(=O)Nc2cc(C(C)(C)O)ccc2Cl)CC1]

1-chloro-N,N,2-trimethylprop-1-en-1-amine (782 mg, 5.86 mmol) was added to a solution of trans-4-({[4-(phenoxycarbonyl)-1H-imidazol-5-yl]carbonyl}amino)cyclohexanecarboxylic acid (70.6 mg, 197 μmol) in dichloromethane (3.7 ml) and the mixture was stirred at room temperature for 30 min. Pyridine (80 μl, 990 μmol) and 2-(3-amino-4-chlorophenyl)propan-2-ol (55.0 mg, 296 μmol) as solution in DMF (0.5 ml) were added and the mixture was stirred over night at room temperature. Methanamine (0.49 ml, 0.99 mmol, 2 M solution in tetrahydrofuran) was added to the reaction and the mixture was stirred for 24 h at room temperature. For work-up, the reaction mixture was concentrated and the residue was purified by preparative HPLC to give the title compound (45.0 mg, 47% yield).

LC-MS (Method 1): Rₜ=0.92 min; MS (ESIpos): m/z=462.3 [M+H]⁺

¹H-NMR (400 MHz, DMSO-d₆): δ [ppm]=13.50-12.63 (m, 1H), 11.81-10.16 (m, 1H), 9.41 (s, 1H), 9.13-8.01 (m, 1H), 7.80 (s, 1H), 7.71 (d, 1H), 7.38 (d, 1H), 7.26 (dd, 1H), 5.13 (s, 1H), 3.86-3.60 (m, 1H), 2.81 (d, 3H), 2.12-1.82 (m, 4H), 1.67-1.22 (m, 10H).

### Example 171

### N⁴-methyl-N⁵-{trans-4-[methyl(phenyl)carbamoyl]cyclohexyl}-1H-imidazole-4,5-dicarboxamide

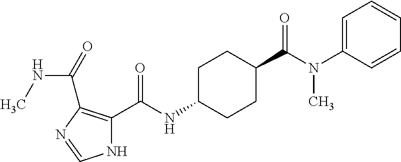 [SMILES: CNC(=O)c1nc[nH]c1C(=O)N[C@H]1CC[C@H](C(=O)N(C)c2ccccc2)CC1]

1-Chloro-N,N,2-trimethylprop-1-en-1-amine (112 mg, 0.839 mmol) was added to a solution of trans-4-({[4-(phenoxycarbonyl)-1H-imidazol-5-yl]carbonyl}amino)cyclohexanecarboxylic acid (100 mg, 280 μmol) in dichloromethane (5.3 ml) and the mixture was stirred at room temperature for 30 min. Pyridine (110 μl, 1.4 mmol) and N-methylaniline (60.0 mg, 560 μmol) were added and the mixture was stirred over night at room temperature followed by 4 h at 400. Then methanamine (700 μl, 1.4 mmol, 2 M solution in tetrahydrofuran) was added and the mixture was stirred for 24 h at room temperature. For work-up, the reaction mixture was concentrated and the residue was purified by preparative HPLC (Method 8) to give the title compound (49.1 mg, 99% purity, 45% yield)

LC-MS (Method 1): Rₜ=0.94 min; MS (ESIpos): m/z=384.2 [M+H]⁺

¹H-NMR (400 MHz, DMSO-d₆): δ [ppm]=13.15 (br. s., 1H), 11.71-10.24 (m, 1H), 9.19-8.02 (m, 1H), 7.76 (s, 1H), 7.56-7.24 (m, 5H), 3.73-3.54 (m, 1H), 3.14 (br. s., 3H), 2.77 (d, 3H), 2.20-2.00 (m, 1H), 1.93-1.58 (m, 4H), 1.56-1.37 (m, 2H), 0.97 (d, 2H).

### Example 172

### N⁵-{trans-4-[(4-fluorophenyl)(methyl)carbamoyl]cyclohexyl}-N⁴-methyl-1H-imidazole-4,5-dicarboxamide

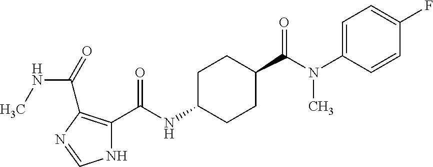 [SMILES: CNC(=O)c1nc[nH]c1C(=O)N[C@H]1CC[C@H](C(=O)N(C)c2ccc(F)cc2)CC1]

1-chloro-N,N,2-trimethylprop-1-en-1-amine (112 mg, 0.839 mmol) was added to a solution of trans-4-({[4-(phenoxycarbonyl)-1H-imidazol-5-yl]carbonyl}amino)cyclohexanecarboxylic acid (100 mg, 280 μmol) in dichloromethane (5.3 ml) and the mixture was stirred at room temperature for 30 min. Pyridine (110 μl, 1.4 mmol) and 4-fluoro-N-methylaniline (70.0 mg, 560 μmol) were added and the mixture was stirred over night at room temperature followed by 4 h at 40° C. Then methanamine (700 μl, 1.4 mmol, 2 M solution in tetrahydrofuran) was added and the mixture was stirred for 24 h at room temperature. For work-up, the reaction mixture was concentrated and the residue was purified by preparative HPLC to give the title compound (23.0 mg, 20% yield).

LC-MS (Method 1): Rₜ=0.96 min; MS (ESIneg): m/z=400.3 [M−H]⁻

¹H-NMR (400 MHz, DMSO-d₆): δ [ppm]=13.15 (br. s., 1H), 11.48-10.27 (m, 1H), 9.11-8.17 (m, 1H), 7.75 (s, 1H), 7.46-7.25 (m, 4H), 3.76-3.55 (m, 1H), 3.11 (br. s, 3H), 2.77 (d, 3H), 2.16-2.03 (m, 1H), 1.93-1.60 (m, 4H), 1.58-1.36 (m, 2H), 1.09-0.89 (m, 2H).

### Example 173

### N⁵-{trans-4-[(2-chloro-4-fluorophenyl)(methyl)carbamoyl]cyclohexyl}-N⁴-methyl-1H-imidazole-4,5-dicarboxamide

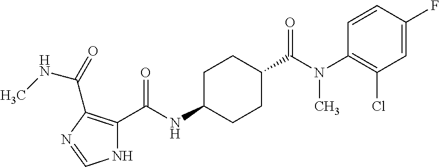 [SMILES: CNC(=O)c1nc[nH]c1C(=O)N[C@H]1CC[C@H](C(=O)N(C)c2ccc(F)cc2Cl)CC1]

### Step 1:

1-Chloro-N,N,2-trimethylprop-1-en-1-amine (134 mg, 1.25 mmol) was added to a solution of trans-4-({[4-(phenoxycarbonyl)-1H-imidazol-5-yl]carbonyl}amino)cyclohexanecarboxylic acid (224 mg, 627 μmol) in dichloromethane (15 ml) and the mixture was stirred at room temperature for 1.5 h. Pyridine (150 μl, 1.9 mmol) and 2-chloro-4-fluoro-N-methylaniline (100 mg, 627 μmol) were added and the mixture was stirred over night at room temperature. For work-up, water was added and the mixture was extracted with ethyl acetate. The combined organic phases were washed with saturated sodium bicarbonate solution and water, filtrated through a silicone filter and concentrated. The residue was purified by flash chromatography (Biotage, 25 g Snap Cartdridge, ethyl acetate/methanol gradient, 0%->10% ethyl acetate) to give phenyl 5-({trans-4-[(2-chloro-4-fluorophenyl)(methyl)carbamoyl]cyclohexyl}carbamoyl)-1H-imidazole-4-carboxylate (135 mg, 43% yield) which was used in the next step.

### Step 2:

Methanamine (810 μl, 2 M solution in tetrahydrofuran, 1.6 mmol) was added to a solution of phenyl 5-({trans-4-[(2-chloro-4-fluorophenyl)(methyl)carbamoyl]cyclohexyl}carbamoyl)-1H-imidazole-4-carboxylate (135 mg, 271 μmol) in tetrahydrofuran (4.0 ml) and the mixture was stirred at room temperature over night. For work-up, water was added and the mixture was extracted with dichloromethane. The combined organic phases were washed with brine, filtrated through a silicone filter and concentrated. The residue was purified by preparative HPLC (Method 9) to give the title compound (37.5 mg, 90% purity, 29% yield).

LC-MS (Method 3): Rₜ=1.00 min; MS (ESIpos): m/z=436.1 [M+H]⁺

¹H-NMR (400 MHz, DMSO-d₆): δ [ppm]=13.40-12.71 (m, 1H), 11.57-10.33 (m, 1H), 8.77-8.05 (m, 1H), 7.76 (s, 1H), 7.73-7.62 (m, 2H), 7.37 (td, 1H), 3.71-3.55 (m, 1H), 3.05 (s, 3H), 2.76 (d, 3H), 1.95-1.32 (m, 7H), 1.21-0.85 (m, 2H).

### Example 174

### N⁵-{trans-4-[(2-chloro-6-cyano-4-fluorophenyl)carbamoyl]cyclohexyl}-N⁴-methyl-1H-imidazole-4,5-dicarboxamide

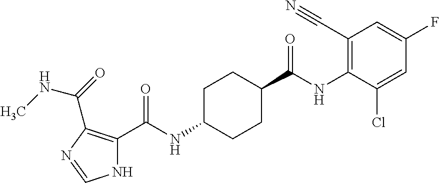 [SMILES: CNC(=O)c1nc[nH]c1C(=O)N[C@H]1CC[C@H](C(=O)Nc2c(Cl)cc(F)cc2C#N)CC1]

1-Chloro-N,N,2-trimethylprop-1-en-1-amine (101 mg, 760 μmol) was added to a solution of trans-4-({[4-(phenoxycarbonyl)-1H-imidazol-5-yl]carbonyl}amino)cyclohexanecarboxylic acid (90.0 mg, 252 μmol) in dichloromethane (4.8 ml) and the mixture was stirred at room temperature for 30 min. Pyridine (100 μl, 1.3 mmol) and 2-amino-3-chloro-5-fluorobenzonitrile (85.9 mg, 504 μmol, CAS No. 1263277-C₆-0) were added and the mixture was stirred over night at room temperature. Then methanamine (630 μl, 2 M solution in tetrahydrofuran, 1.26 mmol) was and the mixture was stirred at room temperature for 24 h. For work-up, the reaction mixture was concentrated and the residue was purified by preparative HPLC (Method 8) to give the title compound (3.30 mg, 97% purity, 3% yield)

LC-MS (Method 1): Rₜ=0.89 min; MS (ESIpos): m/z=447.3 [M+H]⁺

¹H-NMR (400 MHz, DMSO-d₆): δ [ppm]=13.17 (br. s., 1H), 11.41-10.84 (m, 1H), 10.16 (br. s., 1H), 8.86-8.16 (m, 1H), 8.01 (dd, 1H), 7.97 (dd, 1H), 7.80 (s, 1H), 3.87-3.58 (m, 1H), 2.88-2.71 (m, 3H), 2.47-2.39 (m, 1H), 2.14-1.74 (m, 4H), 1.68-1.25 (m, 4H).

### Example 175

### N⁵-{trans-4-[(4-chloro-3-methyl-1,2-thiazol-5-yl)carbamoyl]cyclohexyl}-N⁴-methyl-1H-imidazole-4,5-dicarboxamide

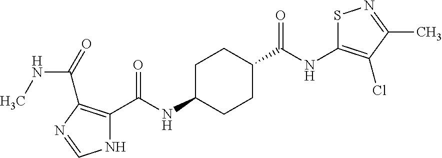

1-Chloro-N,N,2-trimethylprop-1-en-1-amine (135 mg, 1.01 mmol) was added to a solution of trans-4-({[4-(methylcarbamoyl)-1H-imidazol-5-yl]carbonyl}amino)cyclohexanecarboxylic acid (213 mg, 93% purity, 673 μmol) in dichloromethane (15 ml) and the mixture was stirred at room temperature for 30 min. Pyridine (0.160 μl, 2.0 mmol) and 4-chloro-3-methyl-1,2-thiazol-5-amine (100 mg, 673 μmol) were added and the mixture was stirred over night at room temperature. For work-up, water was added, the mixture was extracted with dichloromethane and the organic phase was washed with brine. The organic phase was dried by filtration through a silicone filter and the filtrate was concentrated. The crude product was purified by preparative HPLC (Method 8) to give the title compound (4.00 mg, 99% purity, 1% yield).

LC-MS (Method 1): Rₜ=0.95 min; MS (ESIPos): m/z=425.2 [M+H]

¹H-NMR (400 MHz, DMSO-d₆): δ [ppm]=13.33-13.03 (m, 1H), 11.65-10.94 (m, 2H), 8.68-8.37 (m, 1H), 7.81 (s, 1H), 3.89-3.60 (m, 1H), 2.94-2.71 (m, 4H), 2.31 (s, 3H), 2.13-1.80 (m, 4H), 1.66-1.14 (m, 4H).

### Example 176

### N-{trans-4-[(2-chloro-4-fluoro-5-methylphenyl)(methyl)carbamoyl]cyclohexyl}pyrazolo[1,5-a]pyrimidine-3-carboxamide

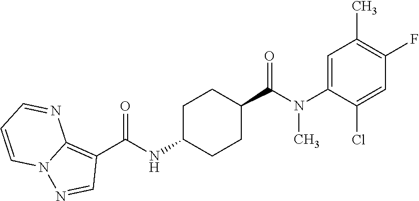 [SMILES: Cc1cc(N(C)C(=O)[C@H]2CC[C@H](NC(=O)c3cnn4cccnc34)CC2)c(Cl)cc1F]

1-Chlor-1-dimethylamino-2-methyl-1-propen (97.3 mg, 0.728 mmol) was added to a solution of trans-4-[(pyrazolo[1,5-a]pyrimidin-3-ylcarbonyl)amino]cyclohexanecarboxylic acid (70.0 mg, 0.243 mmol) in dichloromethane (4.6 ml) and the mixture was stirred at room temperature for 30 min. Pyridine (98 μl, 1.2 mmol) and 2-chloro-4-fluoro-N,5-dimethylaniline (84.3 mg, 486 μmol) were added and the mixture was stirred over night at room temperature and then for 3 h at 40° C. For work-up, the reaction mixture was concentrated and the residue purified by preparative HPLC (Method 8) to give the title compound (67.4 mg, 95% purity, 59% yield)

LC-MS (Method 2): Rₜ=1.15 min; MS (ESIpos): m/z=444.3 [M+H]⁺

¹H-NMR (400 MHz, DMSO-d₆, rotamers present): δ [ppm]=9.32 (dd, 0.1H), 9.28 (dd, 0.9H), 8.82 (dd, 0.1H) 8.73 (dd, 0.9H), 8.58 (s, 0.1H), 8.52 (m, 0.9H), 7.81 (d, 0.1H), 7.68 (d, 0.9H), 7.62 (d, 0.9H), 7.56 (d, 0.9H),7.45 (d, 0.1H), 7.30-7.26 (m, 0.2H), 7.23 (dd, 0.9H), 3.90-3.65 (m, 1H), 3.28 (s, 0.3H), 3.04 (s, 2.7H), 2.25 (d, 2.7H), 2.22 (d, 0.3H), 2.11-1.83 (m, 3H), 1.79-0.76 (m, 6H).

### Example 177

### N-{trans-4-[(2-chloro-4-fluorophenyl)(cyclopropylmethyl)carbamoyl]cyclohexyl}pyrazolo[1,5-a]pyrimidine-3-carboxamide

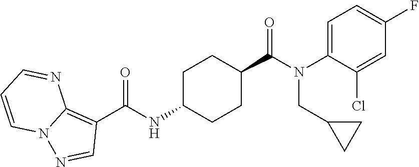 [SMILES: O=C(N[C@H]1CC[C@H](C(=O)N(CC2CC2)c2ccc(F)cc2Cl)CC1)c1cnn2cccnc12]

1-Chlor-1-dimethylamino-2-methyl-1-propen (97.3 mg, 0.728 mmol) was added to a solution of trans-4-[(pyrazolo[1,5-a]pyrimidin-3-ylcarbonyl)amino]cyclohexanecarboxylic acid (70.0 mg, 0.243 mmol) in dichloromethane (4.6 ml) and the mixture was stirred at room temperature for 30 min. Pyridine (98 μl, 1.2 mmol) and 2-chloro-N-(cyclopropylmethyl)-4-fluoroaniline (97.0 mg, 486 μmol) were added and the mixture was stirred over night at room temperature and then for 3 h at 40° C. For work-up, the reaction mixture was concentrated and the residue purified by preparative HPLC (Method 8) to give the title compound (51.8 mg, 96% purity, 44% yield)

LC-MS (Method 1): Rₜ=1.22 min; MS (ESIpos): m/z=470.3 [M+H]⁺

¹H-NMR (400 MHz, DMSO-d₆, rotamers present): δ [ppm]=9.35 (dd, 0.06H), 9.29 (dd, 0.95H), 8.85-8.81 (m, 0.06H), 8.74 (dd, 0.95H), 8.60-8.58 (m, 0.06H), 8.55 (s, 0.95H), 7.83 (d, 0.06H), 7.75-7.67 (m, 1.88H), 7.63 (dd, 1H), 7.58-7.52 (m, 0.08H), 7.44-7.33 (m, 1H), 7.32-7.21 (m, 1.1H), 3.84-3.65 (m, 2H), 3.18 (dd, 1H), 2.00-1.80 (m, 3H), 1.79-1.34 (m, 4H), 1.21-0.76 (m, 3H), 0.48-0.23 (m, 2H), 0.08-0.11 (m, 2H).

### Example 178

### N-{trans-4-[methyl(2-methylphenyl)carbamoyl]cyclohexyl}pyrazolo[1,5-a]pyrimidine-3-carboxamide

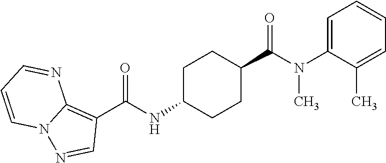 [SMILES: Cc1ccccc1N(C)C(=O)[C@H]1CC[C@H](NC(=O)c2cnn3cccnc23)CC1]

1-Chlor-1-dimethylamino-2-methyl-1-propen (97.3 mg, 0.728 mmol) was added to a solution of trans-4-[(pyrazolo[1,5-a]pyrimidin-3-ylcarbonyl)amino]cyclohexanecarboxylic acid (70.0 mg, 0.243 mmol) in dichloromethane (4.6 ml) and the mixture was stirred at room temperature for 30 min. Pyridine (98 μl, 1.2 mmol) and N,2-dimethylaniline (58.8 mg, 486 μmol, CAS No. 611-21-2) were added and the mixture was stirred over night at room temperature and then for 3 h at 40° C. For work-up, the reaction mixture was concentrated and the residue purified by preparative HPLC (Method 8) to give the title compound (37.6 mg, 97% purity, 38% yield).

LC-MS (Method 2): Rₜ=1.03 min; MS (ESIpos): m/z=392.3 [M+H]⁺

¹H-NMR (400 MHz, DMSO-d₆, rotamers present): 5 [ppm]=9.32 (dd, 0.07H), 9.27 (dd, 0.93H), 8.84-8.81 (m, 0.05H), 8.72 (dd, 0.95H), 8.58 (s, 0.05H), 8.52 (s, 0.95H), 7.81 (d, 0.05H), 7.66 (d, 0.95H), 7.42-7.36 (m, 1H), 7.36-7.07 (m, 4H), 3.88-3.65 (m, 1H), 3.05 (s, 3H), 2.19 (s, 3H), 1.97-1.82 (m, 3H), 1.76-1.36 (m, 4H), 1.11-0.82 (m, 2H).

### Example 179

### N-{trans-4-[(2-chloro-4-fluorophenyl)carbamoyl]-4-methylcyclohexyl}pyrazolo[1,5-a]pyrimidine-3-carboxamide

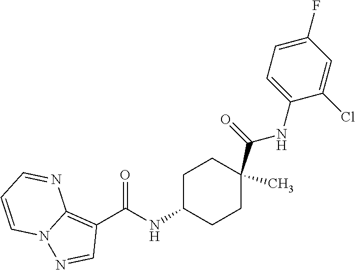 [SMILES: C[C@]1(C(=O)Nc2ccc(F)cc2Cl)CC[C@H](NC(=O)c2cnn3cccnc23)CC1]

(Benzotriazol-1-yloxy)tripyrrolidinophosphonium (181 mg, 348 μmol) was added to a mixture of 4-amino-N-(2-chloro-4-fluorophenyl)-1-methylcyclohexanecarboxamide (mixture of cis-/trans-isomers, intermediate I105) (100 mg, 90% purity, 316 μmol), pyrazolo[1,5-a]pyrimidine-3-carboxylic acid (64.4 mg, 395 μmol, CAS No. 25940-35-6), and N-ethyl-N-isopropylpropan-2-amine (280 μl, 1.6 mmol) in N,N-dimethylformamide (3.5 ml) and the mixture was stirred for 12 h at room temperature. For work-up, the reaction mixture was concentrated under reduced pressure and the residue was purified preparative HPLC (Method 8) to give the title compound (30.0 mg, 99% purity, 17% yield).

LC-MS (Method 1): Rₜ=1.06 min; MS (ESIpos): m/z=403.3 [M+H]⁺

¹H-NMR (400 MHz, DMSO-d₆): δ [ppm]=9.33 (dd, 1H), 9.20 (s, 1H), 8.85 (dd, 1H), 8.57 (s, 1H), 8.02 (d, 1H), 7.52 (dd, 1H), 7.43 (dd, 1H), 7.31-7.18 (m, 2H), 4.02-3.92 (m, 1H), 2.04-1.90 (m, 2H), 1.91-1.79 (m, 2H), 1.72-1.58 (m, 4H), 1.30 (s, 3H).

### Example 180

### N-{cis-4-[(2-chloro-4-fluorophenyl)carbamoyl]-4-methylcyclohexyl}pyrazolo[1,5-a]pyrimidine-3-carboxamide

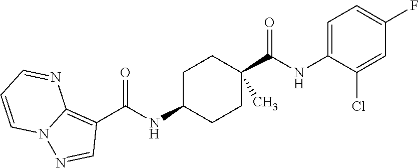 [SMILES: C[C@]1(C(=O)Nc2ccc(F)cc2Cl)CC[C@@H](NC(=O)c2cnn3cccnc23)CC1]

1-Chlor-1-dimethylamino-2-methyl-1-propen (241 mg, 0.1.81 mmol) was added to a solution of 1-methyl-4-[(pyrazolo[1,5-a]pyrimidin-3-ylcarbonyl)amino]cyclohexanecarboxylic acid (isomer 1, intermediate I99) (182 mg, 602 μmol) in dichloromethane (11 ml) and the mixture was stirred at room temperature for 30 min. Pyridine (240 μl, 3.0 mmol) and 2-chloro-4-fluoroaniline (140 μl, 1.2 mmol) were added and the mixture was stirred over night at room temperature. For work-up, water was added and the reaction mixture was extracted with a mixture of dichloromethane/2-propanol (4:1). The combined organic phases were washed with saturated sodium bicarbonate solution and water, filtrated through a silicone filter and concentrated. The residue was purified by preparative HPLC (Method 8) to give the title compound (80.0 mg, 90% purity, 28% yield)

LC-MS (Method 1): Rₜ=1.05 min; MS (ESIpos): m/z=430.2 [M+H]⁺

¹H-NMR (400 MHz, DMSO-d₆): δ [ppm]=9.33-9.25 (m, 2H), 8.80-8.74 (m, 1H), 8.56 (s, 1H), 7.75 (br. d, 1H), 7.53 (dd, 1H), 7.43 (dd, 1H), 7.27-7.21 (m, 2H), 3.93-3.77 (m, 1H), 2.35-2.20 (m, 2H), 1.93-1.79 (m, 2H), 1.56-1.27 (m, 4H), 1.24 (s, 3H).

### Example 181

### N⁴-{cis-4-[(2-chloro-4-fluorophenyl)carbamoyl]-4-methylcyclohexyl}-N⁵-methyl-1H-imidazole-4,5-dicarboxamide

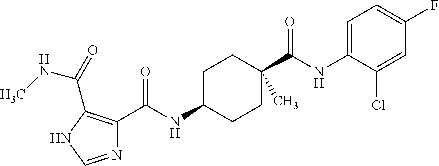 [SMILES: CNC(=O)c1[nH]cnc1C(=O)N[C@H]1CC[C@@](C)(C(=O)Nc2ccc(F)cc2Cl)CC1]

Was prepared in analogy to the synthesis of N-{cis-4-[(2-chloro-4-fluorophenyl)carbamoyl]-4-methylcyclohexyl}pyrazolo[1,5-a]pyrimidine-3-carboxamide (Example 180) using 1-methyl-4-({[5-(methylcarbamoyl)-1H-imidazol-4-yl]carbonyl}amino)cyclohexanecarboxylic acid (isomer 1, intermediate I97) (350 mg, 1.14 mmol) as starting material to give the title compound (129 mg, 26% yield).

LC-MS (Method 1): Rₜ=1.03 min; MS (ESIpos): m/z=436.3 [M+H]⁺

¹H-NMR (400 MHz, DMSO-d₆): δ [ppm]=13.17 (br. s., 1H), 11.36-10.86 (m, 1H), 9.27 (br. s., 1H), 8.76-8.09 (m, 1H), 7.78 (s, 1H), 7.52 (dd, 1H), 7.49-7.33 (m, 1H), 7.24 (td, 1H), 3.85-3.65 (m, 1H), 2.89-2.71 (m, 3H), 2.36-2.19 (m, 2H), 1.90-1.25 (m, 6H), 1.21 (s, 3H).

### Example 182

### N⁵-[(cis)-2-(2-chloro-4-fluorophenyl)-1-oxo-2-azaspiro[4.5]dec-8-yl]-N⁴-methyl-1H-imidazole-4,5-dicarboxamide

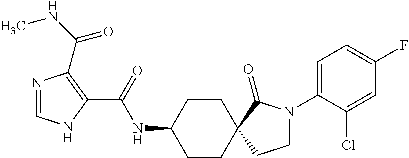 [SMILES: CNC(=O)c1nc[nH]c1C(=O)N[C@H]1CC[C@]2(CCN(c3ccc(F)cc3Cl)C2=O)CC1]

To a solution of 8-amino-2-(2-chloro-4-fluorophenyl)-2-azaspiro[4.5]decan-1-one (intermediate I88) (350 mg, 1.18 mmol) and triethylamine (490 μl, 3.5 mmol) in dichloromethane (13 ml) were added N,N-dimethyl-5,10-dioxo-5H,10H-diimidazo[1,5-a:1′,5′-d]pyrazine-1,6-dicarboxamide (182 mg, 590 μmol) and the mixture was stirred for 24 hours at room temperature. The reaction mixture was concentrated and the residue was purified by flash column chromatography followed by preparative HPLC to give the title compound (35 mg, 93% purity). LC-MS (Method 6): Rₜ=0.99 min; MS (ESIpos): m/z=448.2 [M+H]⁺

¹H-NMR (400 MHz, DMSO-d₆): δ [ppm]=13.18 (br. s., 1H), 11.35 (br. s., 1H), 8.59 (br. s., 1H), 7.80 (s, 1H), 7.59 (dd, 1H), 7.49 (dd, 1H), 7.31 (td, 1H), 3.93 (br. s., 1H), 3.62 (t, 2H), 2.84-2.75 (m, 3H), 2.14-2.04 (m, 2H), 2.03-1.86 (m, 4H), 1.75 (td, 2H), 1.51 (dt, 2H).

### Example 183

### N-[(trans)-2-(2-chloro-4-fluorophenyl)-1-oxo-2-azaspiro[4.5]dec-8-yl]-4-[(6-methyl-1,1-dioxido-1,4-thiazepan-4-yl)carbonyl]-1H-imidazole-5-carboxamide

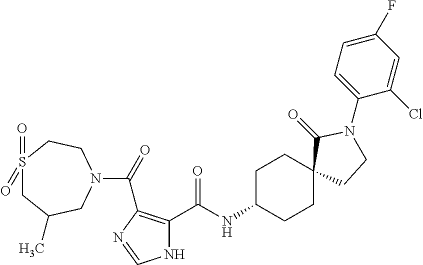 [SMILES: CC1CN(C(=O)c2nc[nH]c2C(=O)N[C@H]2CC[C@@]3(CCN(c4ccc(F)cc4Cl)C3=O)CC2)CCS(=O)(=O)C1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 6-methyl-1,4-thiazepane 1,1-dioxide hydrochloride (78.2 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and diethyl ether was added to the aqueous solution. The resulting precipitate was filtered off and was purified by flash column chromatography to give after drying under vacuum 23 mg of the title compound as a solid material.

LC-MS (Method 1): Rₜ=0.99 min; MS (ESIpos) m/z=580.2 [M+H]⁺.

¹H-NMR (400 MHz, DMSO-d₆), δ [ppm]=13.24 (br. s., 1H), 9.77 (br. s., 1H), 7.82 (s, 1H), 7.59 (dd, 1H), 7.48 (dd, 1H), 7.31 (td, 1H), 4.54-4.10 (m, 1H), 3.72 (br. s., 2H), 3.62 (t, 2H), 3.57-3.43 (m, 2H), 3.32-3.04 (m, 2H), 3.11 (d, 1H), 2.13 (t, 2H), 2.03-1.84 (m, 2H), 1.67 (br. s., 4H), 1.52-1.21 (m, 3H), 1.08 (d, 2H), 0.99-0.75 (m, 2H).

### Example 184

### N-[(trans)-2-(2-chloro-4-fluorophenyl)-1-oxo-2-azaspiro[4.5]dec-8-yl]-4-[(2-methyl-2,6-diazaspiro[3.4]oct-6-yl)carbonyl]-1H-imidazole-5-carboxamide

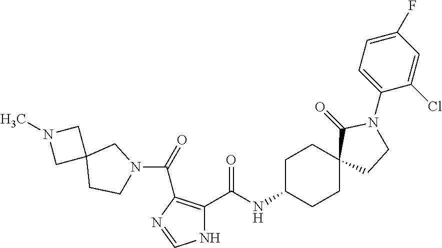 [SMILES: CN1CC2(CCN(C(=O)c3nc[nH]c3C(=O)N[C@H]3CC[C@@]4(CCN(c5ccc(F)cc5Cl)C4=O)CC3)C2)C1]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 2-methyl-2,6-diazaspiro[3.4]octane ethanedioate (84.6 mg, 391 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and the obtained solid was purified by flash column chromatography. The product fractions were concentrated under reduced pressure and diethyl ether was added to the aqueous solution. The resulting precipitate was filtered off to give after drying under vacuum 12 mg of the title compound as a solid material.

LC-MS (Method 1): Rₜ=0.78 min; MS (ESIpos) m/z=543.2 [M+H]⁺.

¹H-NMR (400 MHz, DMSO-d₆), δ [ppm]=13.20 (br. s., 1H), 10.92 (d, 0.5H), 10.85 (d, 0.5H), 7.82 (s, 0.6H), 7.79 (s, 0.4) 7.59 (dd, 1H), 7.52-7.43 (m, 1H), 7.37-7.20 (m, 1H), 4.07 (s, 1H), 3.95 (t, 1H), 3.77-3.66 (m, 1H), 3.66-3.58 (m, 3H), 3.54 (t, 1H), 3.17-3.00 (m, 4H), 2.23 (s, 1.3H), 2.20 (s, 1.7H) 2.14 (t, 2H), 2.08-1.93 (m, 4H), 1.67 (d, 4H), 1.46-1.29 (m, 2H).

### Example 185

### N-[(trans)-2-(2-chloro-4-fluorophenyl)-1-oxo-2-azaspiro[4.5]dec-8-yl]-4-[(6-methyl-2,6-diazaspiro[3.3]hept-2-yl)carbonyl]-1H-imidazole-5-carboxamide

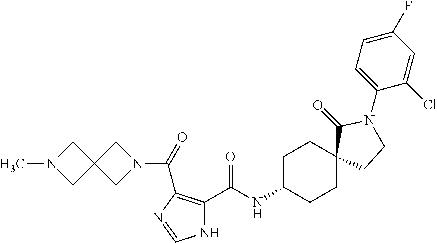 [SMILES: CN1CC2(C1)CN(C(=O)c1nc[nH]c1C(=O)N[C@H]1CC[C@@]3(CCN(c4ccc(F)cc4Cl)C3=O)CC1)C2]

To phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol) in 2 ml tetrahydrofuran were added 2-methyl-2,6-diazaspiro[3.3]heptane ethanedioate (61.5 mg, 196 μmol) and triethylamine (82 μl, 590 μmol) and the mixture was stirred for 90 minutes at room temperature. The reaction mixture was concentrated in vacuo to obtain the crude product, which was purified by HPLC chromatography. The product fractions were concentrated under reduced pressure and diethyl ether was added to the aqueous solution. The resulting precipitate was filtered off and the solid was purified by flash column chromatography to give after drying under vacuum 13 mg of the title compound as a solid material.

LC-MS (Method 1): Rₜ=0.77 min; MS (ESIpos) m/z=529.2 [M+H]⁺.

¹H-NMR (400 MHz, DMSO-d₆), δ [ppm]=13.23 (br. s., 1H), 11.19 (d, 1H), 7.81 (s, 1H), 7.59 (dd, 1H), 7.48 (dd, 1H), 7.32 (td, 1H), 4.65 (s, 2H), 4.13 (s, 2H), 3.77-3.67 (m, 1H), 3.62 (t, 2H), 3.28-3.21 (m, 4H), 2.17 (s, 3H), 2.14 (t, 2H), 2.02-1.89 (m, 2H), 1.72-1.63 (m, 4H), 1.44-1.29 (m, 2H).

### Example 186

### N⁵-[(trans)-2-(2-chloro-4-fluorophenyl)-1-oxo-2-azaspiro[4.5]dec-8-yl]-1H-imidazole-4,5-dicarboxamide

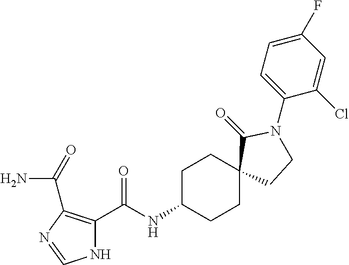 [SMILES: NC(=O)c1nc[nH]c1C(=O)N[C@H]1CC[C@@]2(CCN(c3ccc(F)cc3Cl)C2=O)CC1]

Phenyl 5-{[2-(2-chloro-4-fluorophenyl)-1-oxo-2-azaspiro[4.5]dec-8-yl]carbamoyl}-1H-imidazole-4-carboxylate (isomer 1) (195 mg, 382 μmol) was dissolved in ammonia (19 ml, 0.40 M in THF, 7.6 mmol, Cas No 7664-41-7) and the mixture was stirred for 1 h at room temperature, followed by 4 h at 40° C. The mixture was heated to 80° C. for 2 h and 120° C. for 1 h in a microwave reactor. For work-up, the reaction mixture was concentrated and the residue was purified by preparative HPLC (Method 8). The organic solvent was removed under reduced pressure and the resulting precipitate was collected by filtration and dried under high vacuum to give the title compound (79 mg).

LC-MS (Method 1): Rₜ=0.93 min; MS (ESIpos): m/z=434.1 [M+H]⁺

¹H-NMR (400 MHz, DMSO-d6) δ [ppm]: 1.232 (0.88), 1.382 (1.48), 1.657 (11.87), 1.783 (1.48), 1.986 (1.83), 2.074 (1.92), 2.107 (8.38), 2.124 (16.00), 2.142 (8.66), 2.322 (1.39), 2.326 (1.89), 2.331 (1.32), 2.336 (0.63), 2.518 (5.51), 2.522 (3.50), 2.659 (0.66), 2.664 (1.42), 2.668 (1.92), 2.673 (1.39), 3.600 (8.76), 3.617 (15.59), 3.627 (2.52), 3.634 (8.31), 3.752 (1.26), 7.282 (3.56), 7.289 (3.78), 7.303 (5.98), 7.312 (6.33), 7.325 (4.28), 7.332 (4.57), 7.457 (5.98), 7.471 (6.39), 7.479 (5.17), 7.493 (4.69), 7.573 (7.87), 7.580 (8.00), 7.594 (8.00), 7.601 (7.72), 7.768 (1.95), 7.800 (7.18), 7.971 (1.26), 8.360 (0.72), 10.435 (0.82), 11.291 (1.10), 13.200 (5.01).

### Example 187

### N⁵-[(trans)-2-(2-chloro-4-fluorophenyl)-1-oxo-2-azaspiro[4.5]dec-8-yl]-N⁴-(1-methylazetidin-3-yl)-1H-imidazole-4,5-dicarboxamide

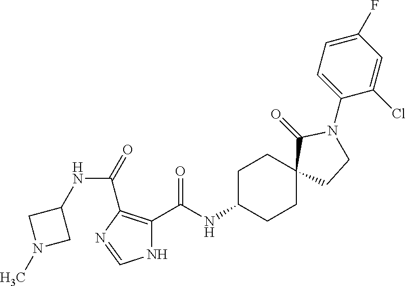 [SMILES: CN1CC(NC(=O)c2nc[nH]c2C(=O)N[C@H]2CC[C@@]3(CCN(c4ccc(F)cc4Cl)C3=O)CC2)C1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (100 mg, 196 μmol), 1-methylazetidin-3-amine dihydrochloride (62.3 mg, 391 μmol, Cas No 959918-41-3) and triethylamine (82 μl) in tetrahydrofuran (1.9 ml) was stirred for 90 min at room temperature. For work-up, the reaction mixture was concentrated and the residue was purified by preparative HPLC, followed by flash chromatography (1 g NH2 Cartridge, dichloromethane/methanol-gradient, 0%->10% methanol) to give the title compound (25 mg, contains 6% diethyl ether).

LC-MS (Method 1): Rₜ=0.81 min; MS (ESIpos): m/z=503.1 [M+H]⁺

1H-NMR (400 MHz, DMSO-d6): δ [ppm]=13.25 (br s, 1H), 11.68-10.86 (m, 1H), 8.98-8.38 (m, 1H), 7.84 (s, 1H), 7.59 (dd, 1H), 7.48 (dd, 1H), 7.31 (td, 1H), 4.58-4.29 (m, 1H), 4.03-3.67 (m, 1H), 3.67-3.45 (m, 4H), 3.16-2.99 (m, 1H), 2.88 (br s, 1H), 2.25 (s, 3H), 2.14 (br t, 2H), 1.97 (br d, 1H), 1.88-1.60 (m, 6H), 1.37 (br d, 1H), 1.23 (s, 1H).

### Example 188

### N-[(trans)-2-(2-chloro-4-fluorophenyl)-1-oxo-2-azaspiro[4.5]dec-8-yl]-4-[(3-methoxyazetidin-1-yl)carbonyl]-1H-imidazole-5-carboxamide

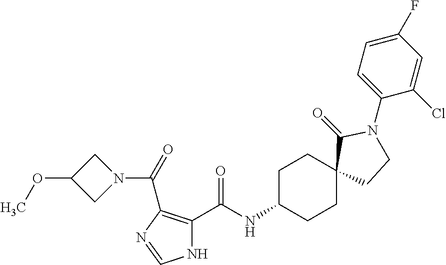 [SMILES: COC1CN(C(=O)c2nc[nH]c2C(=O)N[C@H]2CC[C@@]3(CCN(c4ccc(F)cc4Cl)C3=O)CC2)C1]

To a mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (85.0 mg, 166 μmol) in tetrahydrofuran (1.6 ml), was added 3-methoxyazetidine hydrochloric acid salt (41.1 mg, 333 μmol, Cas No 148644-09-1) and triethylamine (70 μl) and the reaction was stirred for 90 minutes at room temperature. For work-up, the reaction mixture was concentrated and the residue was purified by preparative HPLC (Method 8). The organic solvent was removed under reduced pressure and the resulting precipitate was collected by filtration and dried under high vacuum to give the title compound (48 mg).

LC-MS (Method 1): Rₜ=1.05 min; MS (ESIpos): m/z=504.4 [M+H]⁺

¹H-NMR (400 MHz, DMSO-d6) δ[ppm]: 1.651 (1.33), 1.661 (1.68), 1.675 (1.64), 1.683 (1.59), 2.116 (1.12), 2.133 (2.07), 2.150 (1.18), 3.240 (16.00), 3.599 (1.20), 3.617 (2.17), 3.634 (1.18), 4.245 (1.17), 4.248 (1.53), 7.291 (0.60), 7.306 (0.84), 7.313 (0.95), 7.326 (0.66), 7.333 (0.72), 7.460 (1.14), 7.475 (1.22), 7.482 (1.00), 7.497 (0.93), 7.575 (1.20), 7.582 (1.22), 7.597 (1.19), 7.604 (1.21), 7.809 (5.17), 11.154 (0.90).

### Example 189

### N-[(trans)-2-(2-chloro-4-fluorophenyl)-1-oxo-2-azaspiro[4.5]dec-8-yl]-4-[(3,3-difluoroazetidin-1-yl)carbonyl]-1H-imidazole-5-carboxamide

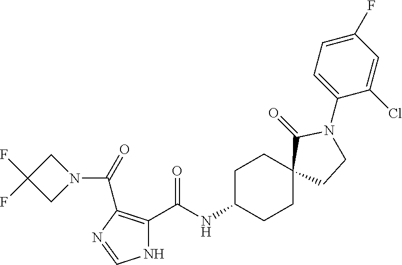 [SMILES: O=C(N[C@H]1CC[C@@]2(CCN(c3ccc(F)cc3Cl)C2=O)CC1)c1[nH]cnc1C(=O)N1CC(F)(F)C1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (85.0 mg, 166 μmol), 3,3-difluoroazetidine hydrochloride (43.1 mg, 333 μmol, Cas No 288315-03-7) and triethylamine (70 μl) in tetrahydrofuran (1.6 ml) was stirred for 5 d at room temperature. For work-up, the reaction mixture was concentrated and the residue was purified by preparative HPLC (Method 8). The organic solvent was removed under reduced pressure and the resulting precipitate was collected by filtration and dried under high vacuum to give the title compound (42 mg).

LC-MS (Method 1): Rₜ=1.17 min; MS (ESIpos): m/z=510.2 [M+H]⁺

¹H-NMR (400 MHz, DMSO-d6) δ[ppm]: 1.349 (1.09), 1.380 (1.84), 1.400 (1.28), 1.410 (1.31), 1.658 (4.48), 1.667 (5.47), 1.680 (4.80), 1.689 (4.68), 1.957 (1.93), 1.966 (2.05), 1.978 (1.35), 1.989 (1.91), 1.998 (1.70), 2.075 (2.77), 2.116 (3.37), 2.133 (6.35), 2.151 (3.56), 2.327 (0.91), 2.518 (2.69), 2.523 (1.94), 2.669 (0.91), 3.600 (3.68), 3.618 (6.31), 3.634 (3.49), 3.731 (0.99), 3.749 (0.99), 4.496 (2.01), 4.527 (3.95), 4.558 (1.87), 4.974 (1.90), 5.006 (3.77), 5.037 (1.78), 7.285 (1.66), 7.292 (1.73), 7.307 (2.43), 7.314 (2.87), 7.327 (1.94), 7.335 (2.04), 7.460 (3.46), 7.475 (3.69), 7.482 (3.02), 7.497 (2.80), 7.575 (3.56), 7.582 (3.48), 7.597 (3.56), 7.604 (3.51), 7.861 (16.00), 10.887 (2.64), 10.905 (2.54), 13.391 (2.66).

### Example 190

### N-[(trans)-2-(2-chloro-4-fluorophenyl)-1-oxo-2-azaspiro[4.5]dec-8-yl]-4-{[3-(difluoromethyl)azetidin-1-yl]carbonyl}-1H-imidazole-5-carboxamide

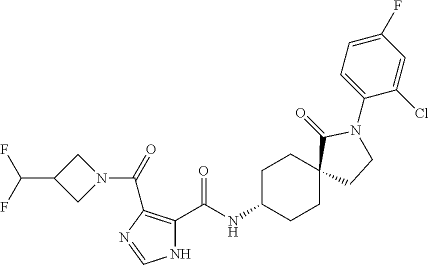 [SMILES: O=C(N[C@H]1CC[C@@]2(CCN(c3ccc(F)cc3Cl)C2=O)CC1)c1[nH]cnc1C(=O)N1CC(C(F)F)C1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (85.0 mg, 166 μmol), 3-(difluoromethyl)azetidine hydrochloride (47.8 mg, 333 μmol, Cas No 1354792-76-9) and triethylamine (70 μl) in tetrahydrofuran (1.6 ml) was stirred for 2 h at room temperature. For work-up, the reaction mixture was concentrated and the residue was purified by preparative HPLC (Method 8). The organic solvent was removed under reduced pressure and the resulting precipitate was collected by filtration and dried under high vacuum to give the title compound (45 mg).

LC-MS (Method 1): Rₜ=1.13 min; MS (ESIpos): m/z=524.2 [M+H]⁺

¹H-NMR (400 MHz, DMSO-d6) δ[ppm]: 1.343 (1.17), 1.354 (1.24), 1.374 (2.00), 1.404 (1.46), 1.653 (4.43), 1.663 (5.74), 1.676 (5.67), 1.685 (5.40), 1.955 (2.24), 1.979 (2.12), 2.118 (3.74), 2.135 (7.17), 2.152 (4.03), 2.327 (0.85), 2.518 (2.63), 2.523 (1.78), 2.669 (0.86), 3.600 (4.11), 3.618 (7.24), 3.635 (3.99), 3.715 (1.12), 3.732 (1.12), 3.978 (1.43), 3.992 (1.47), 4.004 (1.92), 4.019 (1.75), 4.161 (1.68), 4.185 (2.47), 4.210 (1.26), 4.534 (1.38), 4.548 (1.42), 4.562 (1.89), 4.576 (1.78), 4.693 (1.69), 4.715 (2.01), 4.742 (1.24), 6.242 (0.97), 6.253 (0.93), 6.383 (1.78), 6.394 (1.86), 6.535 (0.85), 7.284 (1.92), 7.291 (1.92), 7.306 (2.78), 7.313 (3.25), 7.326 (2.17), 7.334 (2.43), 7.461 (3.92), 7.475 (4.13), 7.482 (3.33), 7.497 (3.08), 7.575 (4.15), 7.582 (4.11), 7.596 (4.11), 7.604 (4.11), 7.826 (16.00), 11.105 (3.31), 11.123 (3.17), 13.290 (3.29).

### Example 192

### N-[(trans)-2-(2-chloro-4-fluorophenyl)-1-oxo-2-azaspiro[4.5]dec-8-yl]-4-[(3-hydroxyazetidin-1-yl)carbonyl]-1H-imidazole-5-carboxamide

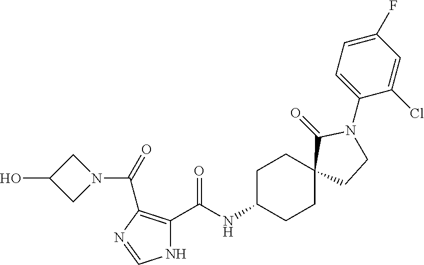 [SMILES: O=C(N[C@H]1CC[C@@]2(CCN(c3ccc(F)cc3Cl)C2=O)CC1)c1[nH]cnc1C(=O)N1CC(O)C1]

To a mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (85.0 mg, 166 μmol), and triethylamine (70 μl) in tetrahydrofuran (1.6 ml) was added azetidin-3-ol hydrochloride (36.5 mg, 333 μmol Cas No 18621-18-6) and the mixture was stirred for 17 h at room temperature. For work-up, the mixture was concentrated and the residue was dissolved in methanol (5 ml), water (50 ml) was added and the mixture was stirred for 1 h, the aqueous phase was concentrated and the resulting precipitate was collected by filtration, washed with water and dried to give the title compound (28 mg).

LC-MS (Method 1): Rₜ=0.90 min; MS (ESIpos): m/z=490.2 [M+H]⁺

¹H-NMR (400 MHz, DMSO-d6) δ[ppm]: 1.233 (1.50), 1.370 (5.34), 1.395 (4.44), 1.662 (16.00), 1.674 (15.94), 1.948 (6.42), 1.979 (6.03), 2.117 (6.87), 2.135 (13.15), 2.151 (7.83), 2.327 (2.04), 2.668 (1.89), 3.601 (7.29), 3.617 (13.15), 3.634 (7.62), 3.725 (3.06), 3.770 (3.84), 3.780 (3.96), 3.797 (4.17), 3.807 (4.08), 4.257 (3.27), 4.275 (4.65), 4.289 (5.58), 4.301 (7.74), 4.329 (4.32), 4.478 (3.99), 4.492 (4.11), 4.777 (3.48), 4.793 (3.69), 4.803 (3.90), 4.820 (3.03), 5.727 (8.35), 5.744 (8.59), 7.283 (2.28), 7.290 (2.85), 7.305 (4.83), 7.312 (5.67), 7.326 (3.18), 7.333 (3.48), 7.460 (4.77), 7.475 (5.61), 7.482 (5.37), 7.497 (4.05), 7.575 (4.95), 7.581 (5.70), 7.596 (5.28), 7.603 (5.61), 7.804 (14.98), 11.201 (6.27), 11.218 (6.21), 13.231 (7.80).

### Example 193

### N-[(trans)-2-(2-chloro-4-fluorophenyl)-1-oxo-2-azaspiro[4.5]dec-8-yl]-4-{[3-(difluoromethoxy)azetidin-1-yl]carbonyl}-1H-imidazole-5-carboxamide

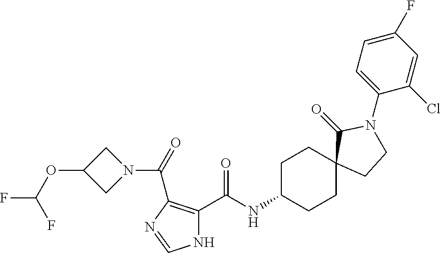 [SMILES: O=C(N[C@H]1CC[C@@]2(CCN(c3ccc(F)cc3Cl)C2=O)CC1)c1[nH]cnc1C(=O)N1CC(OC(F)F)C1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (85.0 mg, 166 μmol), 3-(difluoromethoxy)azetidine (41.0 mg, 333 μmol) and triethylamine (70 μl) in tetrahydrofuran (1.6 ml) was stirred for 17 h at room temperature. For work-up, the reaction mixture was concentrated and the residue was purified by preparative HPLC (Method 8). The organic solvent was removed under reduced pressure and the resulting precipitate was collected by filtration and dried under high vacuum to give the title compound (52 mg).

LC-MS (Method 1): Rₜ=1.14 min; MS (ESIpos): m/z=540.2 [M+H]⁺

¹H-NMR (400 MHz, DMSO-d6) δ[ppm]: 1.373 (1.55), 1.401 (1.27), 1.653 (3.95), 1.663 (5.00), 1.676 (4.78), 1.685 (4.65), 1.953 (1.81), 1.961 (1.65), 1.973 (1.58), 1.984 (1.69), 2.116 (3.24), 2.133 (6.03), 2.151 (3.52), 2.327 (0.73), 2.518 (2.30), 2.523 (1.57), 2.669 (0.73), 3.600 (3.59), 3.617 (6.38), 3.634 (3.43), 4.005 (1.12), 4.014 (1.26), 4.037 (1.43), 4.046 (1.32), 4.406 (1.14), 4.410 (1.15), 4.423 (1.40), 4.426 (1.43), 4.434 (1.18), 4.438 (1.17), 4.455 (1.14), 4.523 (1.07), 4.532 (1.20), 4.556 (1.30), 4.561 (1.36), 4.911 (1.00), 4.927 (1.38), 4.931 (1.43), 4.960 (1.28), 5.002 (1.26), 5.010 (1.33), 5.019 (1.80), 5.028 (1.14), 6.614 (2.63), 6.801 (5.50), 6.988 (2.32), 7.284 (1.69), 7.291 (1.74), 7.306 (2.36), 7.313 (2.83), 7.326 (1.95), 7.333 (2.06), 7.460 (3.37), 7.475 (3.63), 7.482 (2.96), 7.497 (2.75), 7.575 (3.59), 7.582 (3.62), 7.596 (3.60), 7.604 (3.47), 7.826 (16.00), 11.067 (2.59), 11.085 (2.49), 13.297 (2.61).

### Example 194

### N-[(trans)-2-(2-chloro-4-fluorophenyl)-1-oxo-2-azaspiro[4.5]dec-8-yl]-4-(2-oxa-6-azaspiro[3.3]hept-6-ylcarbonyl)-1H-imidazole-5-carboxamide

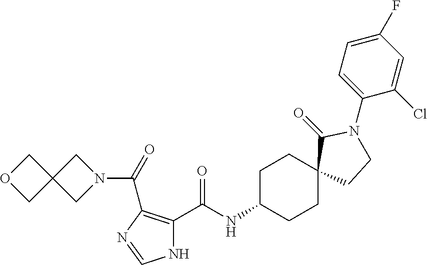 [SMILES: O=C(N[C@H]1CC[C@@]2(CCN(c3ccc(F)cc3Cl)C2=O)CC1)c1[nH]cnc1C(=O)N1CC2(COC2)C1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 1) (85.0 mg, 166 μmol), 2-oxa-6-azaspiro[3.3]heptane ethanedioate (2:1) (48 mg, 166 μmol Cas No 1045709-32-7) and triethylamine (70 μl) in tetrahydrofuran (1.6 ml) was stirred for 2 d at room temperature, followed by 4 h at 50° C. and for 16 hours at 40° C. For work-up, the reaction mixture was concentrated and the residue was purified by preparative HPLC (Method 8). The organic solvent was removed under reduced pressure and the resulting precipitate was collected by filtration and dried under high vacuum to give the title compound (44 mg).

LC-MS (Method 1): Rₜ=0.98 min; MS (ESIpos): m/z=516.3 [M+H]⁺

¹H-NMR (400 MHz, DMSO-d6) δ [ppm]: 1.364 (1.74), 1.393 (1.24), 1.648 (3.78), 1.658 (4.93), 1.672 (4.98), 1.680 (4.81), 1.936 (1.94), 1.944 (2.02), 1.967 (1.93), 1.976 (1.73), 2.115 (3.35), 2.133 (6.33), 2.150 (3.55), 2.327 (0.81), 2.523 (1.50), 2.669 (0.84), 3.600 (3.65), 3.618 (6.26), 3.634 (3.47), 3.704 (1.00), 4.249 (9.17), 4.674 (2.75), 4.691 (11.49), 4.699 (11.42), 4.717 (2.68), 4.768 (8.67), 7.284 (1.64), 7.292 (1.77), 7.306 (2.51), 7.313 (2.81), 7.326 (1.83), 7.334 (2.18), 7.460 (3.54), 7.475 (3.75), 7.482 (3.04), 7.497 (2.80), 7.575 (3.61), 7.582 (3.65), 7.596 (3.54), 7.604 (3.64), 7.811 (16.00), 11.165 (2.72), 11.183 (2.58), 13.242 (2.78).

### Example 195

### N⁴-[(trans)-2-(2-chloro-4-fluorophenyl)-1-oxo-2-azaspiro[4.5]dec-8-yl]-N⁵-(2,2,2-trifluoroethyl)-1H-imidazole-4,5-dicarboxamide

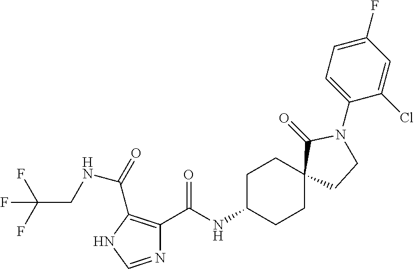 [SMILES: O=C(N[C@H]1CC[C@@]2(CCN(c3ccc(F)cc3Cl)C2=O)CC1)c1nc[nH]c1C(=O)NCC(F)(F)F]

A mixture of (mu-1,4-diazabicyclo[2.2.2]octane-kappaN¹:kappaN⁴)(hexamethyl) dialuminium (282 mg, 1.10 mmol, Cas No 137203-34-0) and 2,2,2-trifluoroethanamine (86 μl, 1.1 mmol Cas No 753-90-2) in tetrahydrofuran (13 ml) was stirred in a sealed tube at 40° C. for 1 h followed by 1 h at room temperature. Phenyl 5-{[2-(2-chloro-4-fluorophenyl)-1-oxo-2-azaspiro[4.5]dec-8-yl]carbamoyl}-1H-imidazole-4-carboxylate (isomer 1) (375 mg, 734 μmol) was added and the mixture was stirred for 1 d at room temperature. For work-up, the mixture was poured into ice-water, extracted with dichloromethane (3×) and the combined organic phases where filtrated through a silicone filter and concentrated. The residue was purified twice by preparative HPLC to give the title compound (20.5 mg).

LC-MS (Method 2): Rₜ=1.07 min; MS (ESIpos): m/z=516 [M+H]⁺

¹H-NMR (400 MHz, DMSO-d₆): δ [ppm]=13.39 (br s, 1H), 7.88 (s, 1H), 7.61-7.57 (m, 1H), 7.48 (dd, 1H), 7.34-7.28 (m, 1H), 4.23-4.09 (m, 2H), 3.80 (br s, 1H), 3.62 (t, 2H), 2.13 (t, 2H), 1.95-1.82 (m, 2H), 1.72-1.45 (m, 6H).

### Example 196

### N⁵-[(trans)-2-(2-chloro-4,5-difluorophenyl)-1-oxo-2-azaspiro[4.5]dec-8-yl]-N⁴-methyl-1H-imidazole-4,5-dicarboxamide

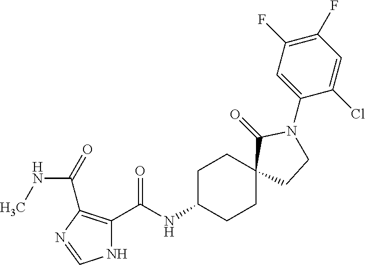 [SMILES: CNC(=O)c1nc[nH]c1C(=O)N[C@H]1CC[C@@]2(CCN(c3cc(F)c(F)cc3Cl)C2=O)CC1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1) (150 mg, 284 μmol) and methanamine (710 μl, 2.0 M in THF, 1.4 mmol in tetrahydrofuran (3.8 ml) was stirred for 5 h at room temperature. For work-up, the reaction mixture was concentrated and the residue was purified by flash chromatography (28 g NH2-cartridge, dichloromethane/methanol-gradient, 0%->10% methanol). The obtained product was recrystallized from diethyl ether give the title compound (94 mg).

LC-MS (Method 1): Rₜ=1.05 min; MS (ESIpos): m/z=466.2 [M+H]⁺

¹H-NMR (400 MHz, DMSO-d6): δ [ppm]=12.94 (br s, 1H), 11.40-10.89 (m, 1H), 8.76-8.18 (m, 1H), 7.88 (dd, 1H), 7.82 (s, 1H), 7.72 (dd, 1H), 3.77 (br s, 1H), 3.65 (t, 2H), 2.83 (br d, 3H), 2.15 (t, 2H), 1.96 (br s, 2H), 1.69 (br s, 5H), 1.46 (br s, 1H).

### Example 197

### N⁵-[(trans)-2-(2-chloro-4,5-difluorophenyl)-1-oxo-2-azaspiro[4.5]dec-8-yl]-N⁴-ethyl-1H-imidazole-4,5-dicarboxamide

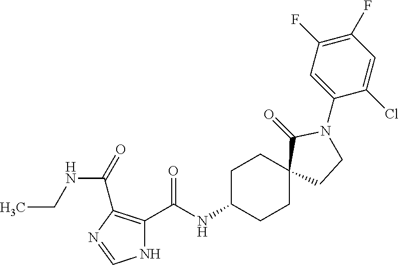 [SMILES: CCNC(=O)c1nc[nH]c1C(=O)N[C@H]1CC[C@@]2(CCN(c3cc(F)c(F)cc3Cl)C2=O)CC1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1) (100 mg, 1.9 mmol) and ethanamine (470 μl, 2.0 M in THF, 950 μmol) in tetrahydrofuran (3.1 ml) was stirred for 8 h at room temperature. For work-up, the reaction mixture was concentrated and the residue was purified by flash chromatography (28 g NH2-cartridge, dichloromethane/methanol-gradient, 0%->10% methanol). The obtained product was recrystallized from diethyl ether give the title compound (48 mg, contains phenol as side product).

LC-MS (Method 1): Rₜ=1.12 min; MS (ESIpos): m/z=480.1 [M+H]⁺

¹H-NMR (600 MHz, DMSO-d6) δ[ppm]: 1.073 (0.41), 1.103 (0.50), 1.114 (0.89), 1.126 (0.50), 1.650 (0.81), 2.119 (0.69), 2.479 (13.27), 2.482 (16.00), 2.485 (12.20), 3.295 (0.75), 3.307 (0.50), 3.608 (0.53), 3.619 (0.95), 3.630 (0.52), 7.783 (1.11).

### Example 198

### N⁵-[(trans)-2-(2-chloro-4,5-difluorophenyl)-1-oxo-2-azaspiro[4.5]dec-8-yl]-N⁴-(propan-2-yl)-1H-imidazole-4,5-dicarboxamide

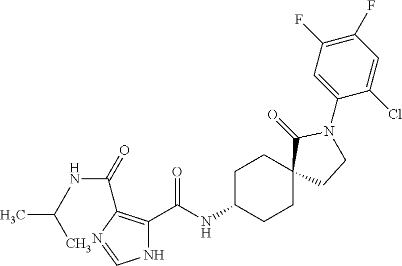 [SMILES: CC(C)NC(=O)c1nc[nH]c1C(=O)N[C@H]1CC[C@@]2(CCN(c3cc(F)c(F)cc3Cl)C2=O)CC1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1)(100 mg, 189 μmol) and propan-2-amine (81 μl, 950 μmol, Cas No 75-31-0) in tetrahydrofuran (3.1 ml) was stirred for 5 h at room temperature. For work-up, the reaction mixture was concentrated and the residue was purified by flash chromatography (28 g NH2-cartridge, dichloromethane/methanol-gradient, 0%->10% methanol). The obtained product was recrystallized from hexane/diethyl ether to give the title compound (59 mg, contains phenol as side product).

LC-MS (Method 1): Rₜ=1.18 min; MS (ESIpos): m/z=494.2 [M+H]⁺

¹H-NMR (400 MHz, DMSO-d6): δ [ppm]=12.91 (br s, 1H), 11.38-10.91 (m, 1H), 8.56-8.23 (m, 1H), 7.91-7.84 (m, 1H), 7.82-7.78 (m, 1H), 7.81-7.77 (m, 1H), 7.70 (dd, 1H), 4.14-3.99 (m, 1H), 3.77 (br s, 1H), 3.62 (t, 2H), 2.12 (t, 2H), 2.02-1.72 (m, 2H), 1.65 (br s, 5H), 1.17 (d, 6H)

### Example 199

### N⁵-[(trans)-2-(2-chloro-4,5-difluorophenyl)-1-oxo-2-azaspiro[4.5]dec-8-yl]-N⁴-cyclopropyl-1H-imidazole-4,5-dicarboxamide

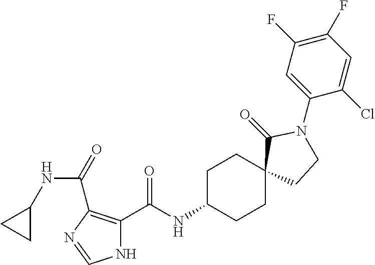 [SMILES: O=C(NC1CC1)c1nc[nH]c1C(=O)N[C@H]1CC[C@@]2(CCN(c3cc(F)c(F)cc3Cl)C2=O)CC1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1) (100 mg, 189 μmol) and cyclopropanamine (65 μl, 950 μmol, Cas No 765-30-0) in tetrahydrofuran (3.1 ml) was stirred for 5 h at room temperature. For work-up, the reaction mixture was concentrated and the residue was purified by flash chromatography (28 g NH2-cartridge, dichloromethane/methanol-gradient, 0%->10% methanol). The obtained product was recrystallized from diethyl ether give the title compound (66 mg, contains phenol as side product).

LC-MS (Method 1): Rₜ=1.13 min; MS (ESIpos): m/z=492.2 [M+H]⁺

¹H-NMR (500 MHz, DMSO-d6) b [ppm]: 0.521 (1.87), 0.721 (5.55), 1.087 (1.41), 1.429 (1.69), 1.658 (16.00), 1.776 (2.05), 1.982 (1.89), 2.134 (8.96), 2.361 (1.47), 2.634 (1.54), 2.864 (3.21), 3.385 (0.88), 3.620 (8.23), 3.634 (14.28), 3.648 (8.21), 3.766 (2.09), 6.734 (7.24), 6.752 (9.66), 6.769 (2.42), 7.137 (4.12), 7.151 (6.98), 7.167 (3.72), 7.694 (3.48), 7.713 (5.22), 7.732 (3.57), 7.806 (8.96), 7.860 (4.14), 7.881 (5.90), 7.897 (4.25), 8.401 (0.88), 8.569 (0.95), 9.324 (5.00), 11.200 (1.21), 13.169 (1.72).

### Example 200

### N-[(trans)-2-(2-chloro-4,5-difluorophenyl)-1-oxo-2-azaspiro[4.5]dec-8-yl]-4-[(3-fluoroazetidin-1-yl)carbonyl]-1H-imidazole-5-carboxamide

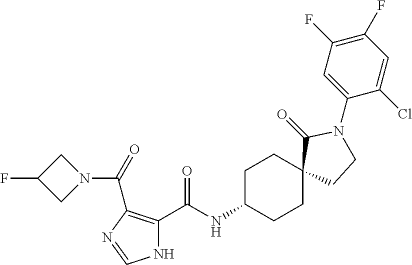 [SMILES: O=C(N[C@H]1CC[C@@]2(CCN(c3cc(F)c(F)cc3Cl)C2=O)CC1)c1[nH]cnc1C(=O)N1CC(F)C1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1) (220 mg, 416 μmol), 3-fluoroazetidine hydrochloride (92.8 mg, 832 μmol, Cas No 617718-46-4) and triethylamine (170 μl) in tetrahydrofuran (6.7 ml) was stirred for 6 h at room temperature. For work-up, the reaction mixture was concentrated and the residue was purified by flash chromatography (28 g NH2-cartridge, dichloromethane/methanol-gradient, 0%->10% methanol). The obtained product was recrystallized from diethyl ether give the title compound (139 mg).

LC-MS (Method 1): Rₜ=1.10 min; MS (ESIpos): m/z=510.2 [M+H]⁺

¹H-NMR (400 MHz, DMSO-d6) δ[ppm]: 0.000 (6.55), 1.357 (1.42), 1.379 (2.20), 1.407 (1.74), 1.658 (5.91), 1.667 (7.13), 1.681 (6.50), 1.689 (6.02), 1.961 (2.53), 1.969 (2.34), 1.983 (2.20), 1.992 (2.27), 2.118 (4.03), 2.136 (7.52), 2.153 (4.14), 3.620 (4.37), 3.627 (1.62), 3.637 (7.80), 3.654 (4.04), 3.716 (1.07), 3.726 (1.31), 3.735 (1.11), 3.743 (1.27), 3.754 (0.98), 4.069 (0.71), 4.076 (1.00), 4.081 (0.73), 4.099 (0.89), 4.104 (1.20), 4.111 (0.92), 4.132 (0.80), 4.138 (1.06), 4.144 (0.73), 4.163 (0.88), 4.168 (1.16), 4.175 (0.78), 4.375 (0.77), 4.379 (0.78), 4.390 (0.92), 4.394 (0.89), 4.405 (0.71), 4.410 (0.75), 4.420 (0.79), 4.425 (0.95), 4.429 (0.96), 4.434 (0.86), 4.444 (0.90), 4.449 (0.85), 4.459 (0.71), 4.464 (0.75), 4.475 (0.64), 4.479 (0.63), 4.590 (0.88), 4.620 (1.08), 4.651 (0.91), 4.681 (1.03), 4.891 (0.68), 4.902 (0.79), 4.943 (0.80), 4.958 (0.77), 5.353 (0.84), 5.360 (1.12), 5.368 (1.29), 5.375 (1.02), 5.382 (0.66), 5.497 (0.75), 5.505 (1.09), 5.512 (1.27), 5.519 (1.05), 5.527 (0.72), 7.685 (2.73), 7.705 (3.10), 7.713 (2.90), 7.733 (2.73), 7.824 (16.00), 7.843 (2.83), 7.863 (3.15), 7.869 (3.14), 7.890 (2.90), 8.314 (0.73), 11.072 (2.33), 11.090 (2.21), 13.295 (2.53).

### Example 201

### N⁵-[(trans)-2-(2-chloro-4,5-difluorophenyl)-1-oxo-2-azaspiro[4.5]dec-8-yl]-N⁴-(2,2,2-trifluoroethyl)-1H-imidazole-4,5-dicarboxamide

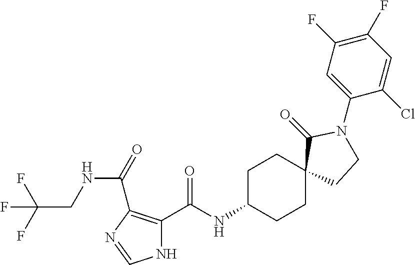 [SMILES: O=C(NCC(F)(F)F)c1nc[nH]c1C(=O)N[C@H]1CC[C@@]2(CCN(c3cc(F)c(F)cc3Cl)C2=O)CC1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1) (150 mg, 284 μmol) and 2,2,2-trifluoroethanamine hydrochloride (192 mg, 1.42 mmol, Cas No 373-88-6) in pyridine (5.0 ml) was heated in a microwave to at 1700 for 90 minutes. For work-up, the reaction mixture was concentrated and the residue was purified by flash chromatography (28 g NH2-cartridge, dichloromethane/methanol-gradient, 0%->10% methanol). The obtained product was recrystallized from diethyl ether give the title compound (76 mg).

LC-MS (Method 6): Rₜ=1.17 min; MS (ESIpos): m/z=534.1 [M+H]⁺

¹H-NMR (400 MHz, CHLOROFORM-d) δ [ppm]: 0.000 (16.00), 1.124 (2.22), 1.141 (4.59), 1.159 (2.39), 1.344 (0.98), 1.367 (2.51), 1.405 (3.31), 1.438 (2.92), 1.473 (2.15), 1.498 (0.97), 1.550 (8.19), 1.663 (3.62), 1.697 (4.88), 1.871 (3.56), 1.880 (4.16), 1.905 (5.92), 1.914 (6.37), 1.939 (2.57), 1.948 (2.57), 2.077 (3.33), 2.096 (8.41), 2.113 (10.88), 2.131 (7.78), 2.151 (6.29), 2.168 (3.36), 3.386 (0.80), 3.403 (2.23), 3.421 (2.21), 3.438 (0.77), 3.598 (8.05), 3.616 (14.04), 3.633 (7.70), 3.905 (0.80), 3.935 (2.16), 3.945 (2.22), 3.954 (2.21), 3.963 (2.13), 3.973 (1.55), 3.985 (1.74), 4.007 (3.00), 4.025 (5.72), 4.040 (4.17), 4.047 (5.53), 4.063 (3.24), 4.084 (0.96), 7.061 (3.06), 7.064 (2.78), 7.080 (3.50), 7.085 (4.76), 7.106 (3.10), 7.109 (2.56), 7.223 (3.29), 7.243 (3.94), 7.246 (4.97), 7.267 (3.25), 7.474 (2.80), 7.494 (2.75), 7.560 (9.35), 7.566 (11.52), 7.890 (1.10), 7.907 (2.16), 7.924 (1.04), 10.716 (2.00), 10.733 (1.97), 11.597 (1.05), 11.791 (0.92), 11.833 (1.48), 11.849 (2.52), 11.864 (1.37).

### Example 202

### N⁵-[(trans)-2-(2-chloro-4,5-difluorophenyl)-1-oxo-2-azaspiro[4.5]dec-8-yl]-N⁴-(2-methoxyethyl)-1H-imidazole-4,5-dicarboxamide

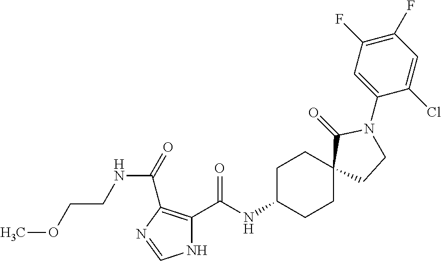 [SMILES: COCCNC(=O)c1nc[nH]c1C(=O)N[C@H]1CC[C@@]2(CCN(c3cc(F)c(F)cc3Cl)C2=O)CC1]

A mixture of phenyl 5-{[2-(2-chloro-4,5-difluorophenyl)-1-oxo-2-azaspiro[4.5]dec-8-yl]carbamoyl}-1H-imidazole-4-carboxylate (isomer 1) (100 mg, 189 μmol) and 2-methoxyethanamine (82 μl, 950 μmol, Cas No 109-85-3) in tetrahydrofuran (3.4 ml) was stirred for 6 h at room temperature. For work-up, the reaction mixture was concentrated and the residue was purified by flash chromatography (28 g NH2-cartridge, dichloromethane/methanol-gradient, 0%->10% methanol). The obtained product was recrystallized from diethyl ether give the title compound (68 mg).

LC-MS (Method 1): Rₜ=1.08 min; MS (ESIpos): m/z=510.2 [M+H]⁺

¹H-NMR (400 MHz, CHLOROFORM-d) δ [ppm]: 0.000 (0.67), 1.124 (1.65), 1.141 (3.58), 1.159 (1.79), 1.438 (0.76), 1.448 (0.72), 1.476 (0.86), 1.572 (1.26), 1.657 (1.18), 1.691 (1.56), 1.863 (0.96), 1.871 (1.13), 1.897 (1.60), 1.905 (1.74), 1.930 (0.70), 1.940 (0.69), 2.092 (2.06), 2.109 (2.12), 2.119 (2.48), 2.127 (1.91), 2.136 (3.04), 2.154 (1.54), 3.341 (16.00), 3.358 (9.08), 3.386 (0.58), 3.403 (1.74), 3.421 (1.71), 3.438 (0.54), 3.495 (0.85), 3.498 (0.94), 3.509 (2.43), 3.521 (2.16), 3.551 (1.65), 3.558 (1.36), 3.562 (1.62), 3.564 (1.67), 3.572 (1.90), 3.587 (3.21), 3.595 (1.89), 3.604 (3.74), 3.612 (2.38), 3.621 (2.05), 3.629 (1.24), 7.060 (1.45), 7.079 (1.58), 7.085 (1.58), 7.104 (1.44), 7.223 (1.51), 7.242 (1.63), 7.247 (1.66), 7.267 (1.46), 7.527 (2.59), 7.539 (4.81), 7.874 (0.85), 11.080 (0.91), 11.098 (0.88).

### Example 203

### N⁴-methyl-N⁵-{trans-4-[methyl(2-methylphenyl)carbamoyl]cyclohexyl}-1H-imidazole-4,5-dicarboxamide

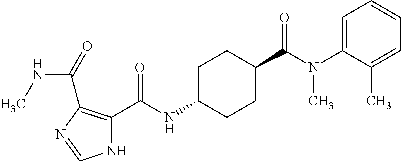 [SMILES: CNC(=O)c1nc[nH]c1C(=O)N[C@H]1CC[C@H](C(=O)N(C)c2ccccc2C)CC1]

1-Chloro-1-dimethylamino-2-methyl-1-propen (161 μl, 1.32 μmol) was added to a solution of trans-4-({[4-(phenoxycarbonyl)-1H-imidazol-5-yl]carbonyl}amino)cyclohexanecarboxylic acid (158 mg, 441 μmol) in dichloromethane (8.3 ml) and the mixture was stirred for 30 minutes at room temperature. N,2-dimethylaniline (107 mg, 881 μmol, CAS 611-21-2) and pyridine (178 μl, 2.2 mmol) were added and the mixture was stirred for 16 h at room temperature followed by 4 h at 40° C. Methanamine (1.1 ml, 2.0 M in tetrahydrofuran, 2.2 mmol) was added and the mixture as stirred at room temperature for 24 h. For work-up, the reaction mixture was concentrated and the residue was purified by flash chromatography (dichloromethane/methanol-gradient, 10%->20% methanol) followed by preparative HPLC [Instrument: Waters Autopurificationsystem; column: Waters XBrigde C18 5μ 100×30 mm; eluent A: water+0.2 Vol-% aqueous ammonia (32%), eluent B: acetonitrile; gradient: 0.00-0.50 min 11% B (25->70 mL/min), 0.51-5.50 min 22-38% B (70 mL/min), DAD scan: 210-400 nm] to give the title compound (52 mg)

LC-MS (Method 2): Rₜ=0.95 min; MS (ESIpos): m/z=398 [M+H]⁺

¹H-NMR (400 MHz, DMSO-d6) δ[ppm]: 1.107 (11.36), 1.305 (0.86), 1.579 (1.64), 2.062 (0.96), 2.185 (14.01), 2.327 (0.74), 2.523 (1.64), 2.669 (0.79), 2.752 (2.66), 2.762 (2.64), 3.049 (16.00), 3.285 (1.36), 4.196 (0.96), 7.254 (0.89), 7.261 (1.09), 7.273 (1.31), 7.277 (2.25), 7.304 (1.85), 7.310 (2.89), 7.319 (2.13), 7.328 (2.82), 7.333 (1.77), 7.383 (1.71), 7.399 (1.08), 7.405 (0.85), 7.754 (6.36).

### Example 204

### N-{trans-4-[(2-chloro-4-fluorophenyl)carbamoyl]cyclohexyl}-7-cyclopropyl-5-methylpyrazolo[1,5-a]pyrimidine-3-carboxamide

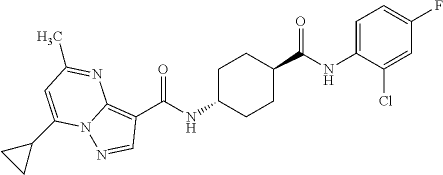 [SMILES: Cc1cc(C2CC2)n2ncc(C(=O)N[C@H]3CC[C@H](C(=O)Nc4ccc(F)cc4Cl)CC3)c2n1]

Benzotriazol-1-yl-oxytripyrrolidinophosphonium hexafluorophosphate (129 mg, 248 μmol) and N,N-diisopropylethylamine (200 μl, 1.1 mmol) were added to a mixture of trans-4-amino-N-(2-chloro-4-fluorophenyl)cyclohexanecarboxamide (61.1 mg, 226 μmol) and 7-cyclopropyl-5-methylpyrazolo[1,5-a]pyrimidine-3-carboxylic acid (123 mg, 282 μmol) in N,N-dimethylformamide (3.4 ml) and the mixture was stirred at room temperature for 12 h. For work-up, water was added and the precipitate formed was collected by filtration, washed with water and methanol. The residue was purified by preparative HPLC [Instrument: Waters Autopurificationsystem; column: Waters XBrigde C18 5μ 100×30 mm; eluent A: water+0.2 Vol-% aqueous ammonia (32%), eluent B: acetonitrile; gradient: 0.00-0.50 min 40% B (25->70 mL/min), 0.51-5.50 min 40-60% B (70 mL/min), DAD scan: 210-400 nm] to give the title compound (3.85 mg).

LC-MS (Method: Instrument: Waters Acquity UPLCMS SingleQuad; column: Acquity UPLC BEH C18 1.7 μm, 50×2.1 mm; eluent A: water+0.2 Vol-% aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm): Rₜ=1.20 min; MS (ESIpos): m/z=470

¹H-NMR (400 MHz, DMSO-d6) δ[ppm]: 1.178 (2.03), 1.185 (2.03), 1.191 (1.96), 1.197 (1.99), 1.207 (0.81), 1.307 (1.02), 1.316 (2.17), 1.323 (1.80), 1.328 (1.39), 1.337 (2.80), 1.344 (2.45), 1.369 (1.07), 1.567 (1.07), 1.941 (1.13), 2.327 (0.76), 2.523 (1.67), 2.579 (16.00), 2.669 (0.76), 2.807 (1.18), 6.833 (5.44), 7.192 (0.74), 7.199 (0.81), 7.212 (1.15), 7.221 (1.18), 7.235 (0.83), 7.242 (0.85), 7.487 (1.39), 7.494 (1.43), 7.509 (1.44), 7.516 (1.39), 7.588 (1.43), 7.603 (1.52), 7.610 (1.37), 7.625 (1.25), 8.002 (1.37), 8.021 (1.34), 8.500 (7.42).

### Example 205

### N-{trans-4-[(2-chloro-4-fluorophenyl)carbamoyl]cyclohexyl}pyrazolo[1,5-a]pyridine-3-carboxamide

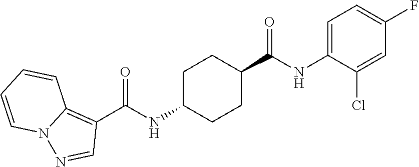 [SMILES: O=C(N[C@H]1CC[C@H](C(=O)Nc2ccc(F)cc2Cl)CC1)c1cnn2ccccc12]

Benzotriazol-1-yl-oxytripyrrolidinophosphonium hexafluorophosphate (161 mg, 310 μmol) and N,N-diisopropylethylamine (230 μl, 1.3 mmol) were added to a mixture of trans-4-amino-N-(2-chloro-4-fluorophenyl)cyclohexanecarboxamide (70 mg, 259 μmol) and pyrazolo[1,5-a]pyridine-3-carboxylic acid (52.4 mg, 323 μmol, Cas No 16205-46-2) in N,N-dimethylformamide (2.9 ml) and the mixture was stirred at room temperature for 12 h. For work-up, the mixture was concentrated, and the residue was stirred with methanol. The precipitated formed was collected by filtration, washed with water and methanol and dried at 50° C. to give the title compound (83.5 mg).

LC-MS (Method 2): Rₜ=1.00 min; MS (ESIpos): m/z=415 [M+H]⁺

¹H-NMR (600 MHz, DMSO-d₆): δ [ppm]=9.49 (s, 1H), 8.76 (d, 1H), 8.61-8.57 (m, 1H), 8.22 (d, 1H), 7.98 (br d, 1H), 7.63 (dd, 1H), 7.51 (dd, 1H), 7.49-7.44 (m, 1H), 7.23 (td, 1H), 7.06 (td, 1H), 3.87-3.78 (m, 1H), 2.50-2.44 (m, 1H), 2.03-1.93 (m, 4H), 1.61-1.52 (m, 2H), 1.46-1.36 (m, 2H)

### Example 206

### N-{trans-4-[(2-chloro-4-fluorophenyl)carbamoyl]cyclohexyl}imidazo[1,2-a]pyridine-3-carboxamide

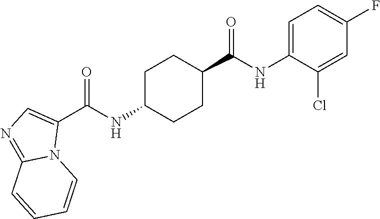 [SMILES: O=C(N[C@H]1CC[C@H](C(=O)Nc2ccc(F)cc2Cl)CC1)c1cnc2ccccn12]

Was prepared in analogy to the synthesis of N-{trans-4-[(2-chloro-4-fluorophenyl)carbamoyl]cyclohexyl}pyrazolo[1,5-a]pyridine-3-carboxamide using imidazo[1,2-a]pyridine-3-carboxylic acid (52.4 mg, 323 μmol, Cas No 6200-60-8) as starting material to give the title compound (59 mg).

LC-MS (Method 2): Rₜ=1.00 min; MS (ESIpos): m/z=415 [M+H]⁺

¹H-NMR (600 MHz, DMSO-d₆): δ [ppm]=9.58 (d, 1H), 9.50 (s, 1H), 8.53 (s, 1H), 8.45 (br d, 1H), 7.85 (d, 1H), 7.69 (br t, 1H), 7.64 (dd, 1H), 7.52 (dd, 1H), 7.32 (br t, 1H), 7.24 (td, 1H), 3.90-3.82 (m, 1H), 2.00 (br t, 4H), 1.63-1.54 (m, 2H), 1.50-1.39 (m, 2H)

### Example 207

### N-[(cis)-2-(2-chloro-4-fluorophenyl)-1-oxo-2-azaspiro[4.5]dec-8-yl]-5,7-dimethylpyrazolo[1,5-a]pyrimidine-3-carboxamide

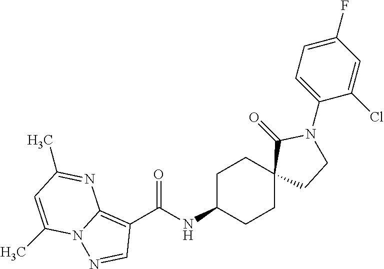 [SMILES: Cc1cc(C)n2ncc(C(=O)N[C@H]3CC[C@]4(CCN(c5ccc(F)cc5Cl)C4=O)CC3)c2n1]

Benzotriazol-1-yl-oxytripyrrolidinophosphonium hexafluorophosphate (637 mg, 1.22 mmol) and N,N-diisopropylethylamine (970 μl, 5.6 mmol) were added to a mixture of 8-amino-2-(2-chloro-4-fluorophenyl)-2-azaspiro[4.5]decan-1-one (isomer 2) (298 mg, 1.11 mmol) and 5,7-dimethylpyrazolo[1,5-a]pyrimidine-3-carboxylic acid (266 mg, 1.39 mmol, Cas No 90349-23-8) in N,N-dimethylformamide (12 ml) and the mixture was stirred at room temperature for 12 h. For work-up, the mixture was concentrated and the residue was purified by flash chromatography (ethyl acetate/methanol gradient, 0%->20% methanol) followed by preparative HPLC [Instrument: Labomatic HD5000, Labocord-5000; Gilson GX-241, Labcol Vario 4000, column: Chiralpak IA 5μ 250×30 mm; eluent A: hexane+0.1 vol-% diethyl amine; eluent B: 2-propanol; isocratic: 50% A+50% B; flow 40.0 ml/min; detection: UV 254 nm] to give N-[(cis)-2-(2-chloro-4-fluorophenyl)-1-oxo-2-azaspiro[4.5]dec-8-yl]-5,7-dimethylpyrazolo[1,5-a]pyrimidine-3-carboxamide (83 mg) and N-[(cis)-2-(4-fluorophenyl)-1-oxo-2-azaspiro[4.5]dec-8-yl]-5,7-dimethylpyrazolo[1,5-a]pyrimidine-3-carboxamide (141 mg).

### N-[(cis)-2-(2-chloro-4-fluorophenyl)-1-oxo-2-azaspiro[4.5]dec-8-yl]-5,7-dimethylpyrazolo[1,5-a]pyrimidine-3-carboxamide

HPLC: [Instrument: Agilent HPLC 1260; column: Chiralpak IA 3μ 100×4.6 mm; eluent A: hexane+0.1 vol-% diethyl amine; eluent B: 2-propanol; isocratic: 50% A+50% B; flow 1.4 ml/min; temperature: 25° C.; detection: DAD @ 254 nm]: 3.75 min

LC-MS (Method 6): Rₜ=1.14 min; MS (ESIpos): m/z=470.2 [M+H]⁺

¹H-NMR (400 MHz, DMSO-d₆): δ [ppm]=8.62 (br d, 1H), 8.49 (s, 1H), 7.60 (dd, 1H), 7.50 (dd, 1H), 7.32 (td, 1H), 7.13-7.11 (m, 1H), 4.24-4.18 (m, 1H), 3.64 (t, 2H), 2.74 (s, 3H), 2.65 (s, 3H), 2.15 (t, 2H), 2.02-1.86 (m, 2H), 1.85-1.75 (m, 4H), 1.62-1.50 (m, 2H)

### Example 208

### N-[(cis)-2-(4-fluorophenyl)-1-oxo-2-azaspiro[4.5]dec-8-yl]-5,7-dimethyl pyrazolo[1,5-a]pyrimidine-3-carboxamide

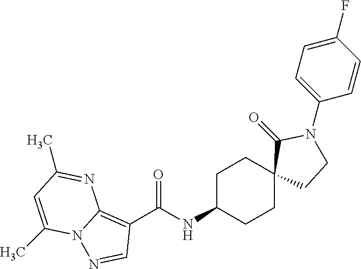 [SMILES: Cc1cc(C)n2ncc(C(=O)N[C@H]3CC[C@]4(CCN(c5ccc(F)cc5)C4=O)CC3)c2n1]

Isolated as side-product in the synthesis of N-[(cis)-2-(2-chloro-4-fluorophenyl)-1-oxo-2-azaspiro[4.5]dec-8-yl]-5,7-dimethylpyrazolo[1,5-a]pyrimidine-3-carboxamide (141 mg).

HP-LC: [Instrument: Agilent HPLC 1260; column: Chiralpak IA 3μ 100×4.6 mm; eluent A: hexane+0.1 vol-% diethyl amine; eluent B: 2-propanol; isocratic: 50% A+50% B; flow 1.4 ml/min; temperature: 25° C.; detection: DAD @ 254 nm]: 6.12 min

LC-MS (Method 6): Rₜ=1.14 min; MS (ESIpos): m/z=436.2 [M+H]⁺

¹H-NMR (400 MHz, DMSO-d₆): δ [ppm]=8.65 (br d, 1H), 8.49 (s, 1H), 7.74-7.68 (m, 2H), 7.26-7.19 (m, 2H), 7.14 (s, 1H), 4.24-4.18 (m, 1H), 3.83-3.75 (m, 2H), 2.75 (s, 3H), 2.70 (s, 3H), 2.11-1.99 (m, 2H), 1.99-1.88 (m, 2H), 1.85-1.72 (m, 4H), 1.55-1.47 (m, 2H)

### Example 209

### N-[(trans)-2-(4-fluorophenyl)-1-oxo-2-azaspiro[4.5]dec-8-yl]pyrazolo[1,5-a]pyrimidine-3-carboxamide

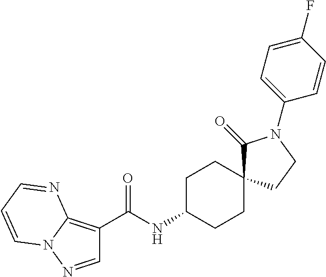 [SMILES: O=C(N[C@H]1CC[C@@]2(CCN(c3ccc(F)cc3)C2=O)CC1)c1cnn2cccnc12]

Benzotriazol-1-yl-oxytripyrrolidinophosphonium hexafluorophosphate (193 mg, 371 μmol) and N,N-diisopropylethylamine (270 μl, 1.5 mmol) were added to a mixture of 8-amino-2-(4-fluorophenyl)-2-azaspiro[4.5]decan-1-one (isomer 1) (90.0 mg, 309 μmol) and pyrazolo[1,5-a]pyrimidine-3-carboxylic acid (63.0 mg, 386 μmol, Cas No 25940-35-6) in N,N-dimethylformamide (3.4 ml) and the mixture was stirred at room temperature for 12 h. For work-up, the mixture was concentrated, and the residue was stirred with methanol. The precipitated formed was collected by filtration, washed with water and methanol and dried at 50° C. to give the title compound (74 mg).

LC-MS (Method 2): Rₜ=1.03 min; MS (ESIpos): m/z=408.5 [M+H]⁺

¹H-NMR (400 MHz, DMSO-d6) δ[ppm]: 1.458 (2.05), 1.468 (2.12), 1.487 (2.31), 1.497 (2.45), 1.518 (1.12), 1.615 (1.32), 1.650 (7.07), 1.659 (5.36), 1.683 (2.58), 1.691 (2.98), 1.940 (2.51), 1.949 (2.64), 1.972 (2.45), 1.981 (2.18), 2.080 (4.10), 2.098 (7.40), 2.115 (4.30), 2.322 (0.93), 2.326 (1.19), 2.332 (0.86), 2.523 (2.78), 2.664 (0.86), 2.669 (1.26), 2.673 (0.93), 2.888 (0.73), 3.772 (4.43), 3.789 (7.74), 3.807 (4.56), 3.832 (1.32), 3.851 (1.26), 5.758 (1.12), 7.200 (5.16), 7.206 (1.65), 7.218 (2.25), 7.223 (8.73), 7.227 (2.38), 7.239 (1.79), 7.245 (5.62), 7.259 (4.10), 7.270 (3.97), 7.277 (4.03), 7.288 (4.17), 7.700 (5.42), 7.706 (2.12), 7.713 (5.75), 7.718 (3.17), 7.724 (5.29), 7.730 (1.92), 7.736 (4.89), 7.813 (3.44), 7.832 (3.37), 8.579 (16.00), 8.818 (4.69), 8.822 (5.02), 8.828 (4.69), 8.832 (4.56), 9.308 (4.76), 9.312 (5.02), 9.326 (4.96), 9.330 (4.56).

### Example 210

### N-[(trans)-2-(4-fluorophenyl)-1-oxo-2-azaspiro[4.5]dec-8-yl]-6-methylpyrazolo[1,5-a]pyrimidine-3-carboxamide

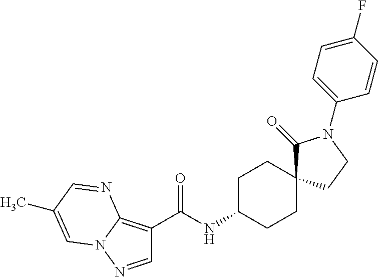 [SMILES: Cc1cnc2c(C(=O)N[C@H]3CC[C@@]4(CCN(c5ccc(F)cc5)C4=O)CC3)cnn2c1]

Was prepared in analogy to the synthesis of N-[(trans)-2-(4-fluorophenyl)-1-oxo-2-azaspiro[4.5]dec-8-yl]pyrazolo[1,5-a]pyrimidine-3-carboxamide using 6-methylpyrazolo[1,5-a]pyrimidine-3-carboxylic acid (68.4 mg, 386 μmol, Cas No 869941-96-8) as coupling partner to give the title compound (115 mg).

LC-MS (Method 2): Rₜ=1.10 min; MS (ESIpos): m/z=422.5 [M+H]⁺

¹H-NMR (400 MHz, DMSO-d6) δ[ppm]: 1.450 (1.54), 1.461 (1.49), 1.480 (1.64), 1.490 (1.64), 1.648 (4.87), 1.657 (3.47), 1.681 (1.83), 1.689 (1.98), 1.936 (1.78), 1.945 (1.83), 1.968 (1.69), 1.978 (1.49), 2.080 (2.94), 2.097 (5.16), 2.114 (2.94), 2.323 (1.06), 2.327 (1.35), 2.332 (1.11), 2.381 (16.00), 2.383 (15.42), 2.518 (2.75), 2.523 (1.93), 2.669 (0.96), 2.727 (1.98), 2.888 (2.36), 3.269 (1.98), 3.770 (3.28), 3.788 (5.54), 3.805 (3.33), 7.200 (3.76), 7.206 (1.30), 7.218 (1.64), 7.223 (6.17), 7.228 (1.45), 7.239 (1.30), 7.245 (4.14), 7.700 (4.10), 7.706 (1.54), 7.713 (4.29), 7.718 (2.27), 7.724 (3.95), 7.730 (1.45), 7.736 (3.61), 7.759 (2.51), 7.778 (2.41), 8.491 (13.98), 8.727 (4.92), 8.732 (4.87), 9.174 (3.28), 9.177 (3.90), 9.179 (4.00), 9.182 (3.04).

### Example 211

### N-[(trans)-2-(4-fluorophenyl)-1-oxo-2-azaspiro[4.5]dec-8-yl]-5,7-di methyl pyrazolo[1,5-a]pyrimidine-3-carboxamide

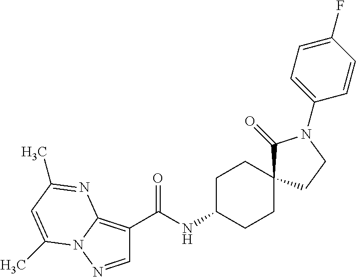 [SMILES: Cc1cc(C)n2ncc(C(=O)N[C@H]3CC[C@@]4(CCN(c5ccc(F)cc5)C4=O)CC3)c2n1]

Was prepared in analogy to the synthesis of N-[(trans)-2-(4-fluorophenyl)-1-oxo-2-azaspiro[4.5]dec-8-yl]pyrazolo[1,5-a]pyrimidine-3-carboxamide using 5,7-dimethylpyrazolo[1,5-a]pyrimidine-3-carboxylic acid (73.8 mg, 386 μmol Cas No 90349-23-8) as coupling partner to give the title compound (99 mg).

LC-MS (Method 2): Rₜ=1.16 min; MS (ESIneg): m/z=434.5 [M−H]⁻

¹H-NMR (400 MHz, DMSO-d6) δ[ppm]: 1.657 (2.51), 1.667 (1.93), 1.691 (1.11), 1.699 (1.21), 2.086 (1.63), 2.103 (3.00), 2.121 (1.74), 2.518 (3.20), 2.522 (2.72), 2.631 (16.00), 2.735 (9.29), 2.737 (9.66), 3.372 (2.39), 3.774 (1.93), 3.792 (3.39), 3.809 (2.04), 7.123 (3.02), 7.126 (3.16), 7.200 (2.09), 7.223 (3.55), 7.245 (2.32), 7.702 (2.16), 7.714 (2.37), 7.720 (1.25), 7.725 (2.18), 7.738 (2.02), 8.008 (1.37), 8.027 (1.35), 8.496 (6.83).

### Example 212

### N⁴-[(trans)-2-(4-fluorophenyl)-1-oxo-2-azaspiro[4.5]dec-8-yl]-N⁵-methyl-1H-imidazole-4,5-dicarboxamide

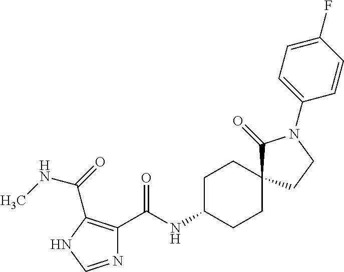 [SMILES: CNC(=O)c1[nH]cnc1C(=O)N[C@H]1CC[C@@]2(CCN(c3ccc(F)cc3)C2=O)CC1]

A mixture of phenyl 4-{[2-(4-fluorophenyl)-1-oxo-2-azaspiro[4.5]dec-8-yl]carbamoyl}-1H-imidazole-5-carboxylate (isomer 1) (121 mg, 80% purity, 203 μmol) and methanamine (0.51 ml, 2.0 M in tetrahydrofuran, 1.0 mmol) in tetrahydrofuran (1.4 ml) was stirred for 1 d at room temperature. For work-up, the reaction mixture was concentrated and the residue was purified by preparative HPLC (Method 9) to give the title compound (43 mg).

LC-MS (Method 3): Rₜ=0.98 min; MS (ESIpos): m/z=414.4 [M+H]⁺

¹H-NMR (500 MHz, DMSO-d₆): δ [ppm]=13.11 (br s, 1H), 11.83-10.54 (m, 1H), 9.14-8.10 (m, 1H), 7.80 (s, 1H), 7.73-7.69 (m, 2H), 7.25-7.19 (m, 2H), 3.82-3.72 (m, 3H), 2.81 (d, 3H), 2.07 (t, 2H), 1.97-1.80 (m, 2H), 1.71-1.60 (m, 4H), 1.47 (br s, 2H)

### Example 213

### N⁵-ethyl-N⁴-[(trans)-2-(4-fluorophenyl)-1-oxo-2-azaspiro[4.5]dec-8-yl]-1H-imidazole-4,5-dicarboxamide

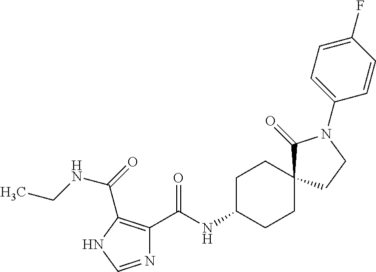 [SMILES: CCNC(=O)c1[nH]cnc1C(=O)N[C@H]1CC[C@@]2(CCN(c3ccc(F)cc3)C2=O)CC1]

Was prepared in analogy to the synthesis of N⁴-[(trans)-2-(4-fluorophenyl)-1-oxo-2-azaspiro[4.5]dec-8-yl]-N⁵-methyl-1H-imidazole-4,5-dicarboxamide using ethanamine (510 μl, 2.0 M in tetrahydrofuran, 1.0 mmol) as coupling partner to give the title compound (56 mg).

LC-MS (Method 3): Rₜ=1.06 min; MS (ESIpos): m/z=428.4 [M+H]⁺

¹H-NMR (500 MHz, DMSO-d6) δ[ppm]: 1.115 (7.47), 1.129 (15.52), 1.143 (7.68), 1.631 (8.78), 1.657 (2.87), 2.057 (4.31), 2.071 (7.84), 2.085 (4.49), 2.357 (0.77), 2.361 (1.07), 2.365 (0.82), 2.518 (3.19), 2.522 (2.35), 2.539 (1.32), 2.634 (1.05), 2.638 (0.80), 3.282 (1.51), 3.296 (4.92), 3.308 (5.95), 3.311 (6.08), 3.323 (7.86), 3.768 (6.08), 3.783 (9.85), 3.796 (5.42), 7.201 (6.16), 7.206 (2.08), 7.215 (2.64), 7.219 (10.32), 7.223 (2.82), 7.232 (2.07), 7.236 (6.31), 7.697 (6.22), 7.702 (2.42), 7.707 (6.82), 7.712 (3.76), 7.716 (6.22), 7.721 (2.30), 7.726 (5.63), 7.805 (16.00).

### Example 214

### N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide

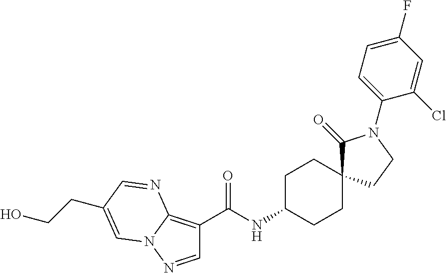 [SMILES: O=C(N[C@H]1CC[C@@]2(CCN(c3ccc(F)cc3Cl)C2=O)CC1)c1cnn2cc(CCO)cnc12]

Benzotriazol-1-yl-oxytripyrrolidinophosphonium hexafluorophosphate (206 mg, 396 μmol) and N,N-diisopropylethylamine (290 μl, 1.7 mmol) were added to a mixture of 8-amino-2-(2-chloro-4-fluorophenyl)-2-azaspiro[4.5]decan-1-one (isomer 1) (100 mg, 330 μmol) and 6-(2-hydroxyethyl)pyrazolo[1,5-a]pyrimidine-3-carboxylic acid (85.5 mg, 413 μmol, Cas No 725693-84-5) in N,N-dimethylformamide (3.7 ml) and the mixture was stirred at room temperature for 12 h. For work-up, the mixture was concentrated and the residue was purified by preparative HPLC (Method 8) to give the title compound (7.5 mg)

LC-MS (Method 2): Rₜ=0.98 min; MS (ESIpos): m/z=486.2 [M+H]⁺

¹H-NMR (400 MHz, DMSO-d6) δ[ppm]: 1.460 (1.28), 1.483 (2.05), 1.503 (1.71), 1.670 (6.35), 1.679 (7.10), 1.695 (4.79), 1.965 (2.43), 1.988 (2.24), 2.074 (1.15), 2.148 (3.70), 2.166 (7.19), 2.183 (3.95), 2.298 (1.34), 2.323 (1.40), 2.327 (1.90), 2.331 (1.40), 2.523 (4.26), 2.665 (1.40), 2.669 (1.90), 2.674 (1.34), 2.831 (3.08), 2.846 (6.63), 2.862 (3.30), 3.609 (3.95), 3.627 (7.00), 3.643 (3.77), 3.669 (1.99), 3.685 (4.98), 3.698 (4.86), 3.714 (1.84), 3.828 (1.12), 3.847 (1.12), 4.782 (2.15), 4.795 (5.01), 4.809 (2.18), 7.288 (1.53), 7.295 (1.68), 7.309 (2.65), 7.317 (2.89), 7.331 (1.87), 7.338 (2.05), 7.466 (3.46), 7.481 (3.70), 7.488 (2.99), 7.503 (2.77), 7.579 (3.46), 7.586 (3.58), 7.600 (3.55), 7.607 (3.49), 7.794 (3.33), 7.814 (3.24), 8.512 (16.00), 8.762 (6.91), 8.767 (6.88), 9.157 (5.85), 9.162 (5.70).

### Example 215

### N-[(trans)-2-(2-chloro-4-fluorophenyl)-1-oxo-2-azaspiro[4.5]dec-8-yl]-5-(methylamino)pyrazolo[1,5-a]pyrimidine-3-carboxamide

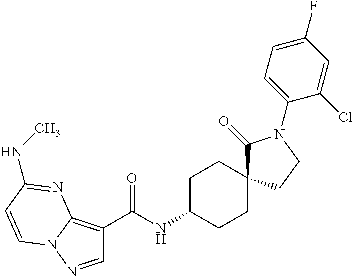 [SMILES: CNc1ccn2ncc(C(=O)N[C@H]3CC[C@@]4(CCN(c5ccc(F)cc5Cl)C4=O)CC3)c2n1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 5-(methylamino)pyrazolo[1,5-a]pyrimidine-3-carboxylic acid (80.9 mg, 421 μmol) as coupling partner. For work-up the reaction mixture was concentrated, the residue was stirred with methanol and the precipitate was collected by filtration. The thus obtained solid was purified by preparative HPLC (Method 9) to give the title compound (15 mg).

LC-MS (Method 2): Rₜ=1.01 min; MS (ESIpos): m/z=471 [M+H]⁺

¹H-NMR (600 MHz, DMSO-d6) δ[ppm]: 1.358 (0.93), 1.369 (2.19), 1.377 (2.29), 1.389 (2.50), 1.397 (2.27), 1.409 (1.13), 1.417 (0.95), 1.660 (1.13), 1.675 (7.34), 1.681 (10.28), 1.695 (3.66), 1.700 (3.84), 1.717 (0.88), 2.042 (2.81), 2.057 (2.71), 2.063 (2.50), 2.072 (5.59), 2.123 (4.56), 2.134 (8.43), 2.146 (4.77), 2.381 (1.29), 2.384 (1.78), 2.387 (1.29), 2.474 (1.93), 2.477 (2.94), 2.480 (3.71), 2.482 (3.92), 2.485 (4.46), 2.515 (7.63), 2.518 (6.70), 2.521 (5.23), 2.526 (2.01), 2.609 (1.47), 2.612 (1.91), 2.615 (1.37), 2.923 (15.51), 2.931 (16.00), 3.164 (7.09), 3.173 (7.11), 3.287 (0.82), 3.327 (0.88), 3.609 (4.95), 3.614 (1.60), 3.620 (8.86), 3.631 (4.87), 3.727 (1.24), 3.734 (1.52), 3.739 (1.29), 3.746 (1.52), 3.753 (1.19), 4.074 (1.78), 4.082 (1.75), 6.355 (3.45), 6.368 (3.53), 7.289 (1.65), 7.294 (1.91), 7.303 (3.12), 7.308 (3.30), 7.318 (2.01), 7.322 (2.19), 7.463 (3.53), 7.472 (3.89), 7.477 (3.40), 7.487 (3.07), 7.571 (3.56), 7.575 (3.53), 7.585 (3.61), 7.590 (3.50), 7.990 (2.65), 8.002 (2.58), 8.076 (11.98), 8.533 (3.22), 8.545 (3.27).

### Example 216

### N-[(trans)-2-(2-chloro-4-fluorophenyl)-1-oxo-2-azaspiro[4.5]dec-8-yl]-2-methylpyrazolo[1,5-a]pyrimidine-3-carboxamide

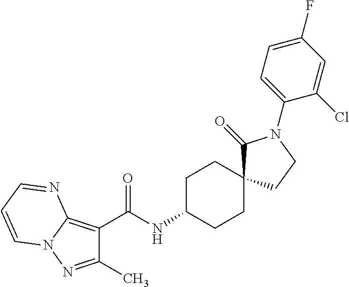 [SMILES: Cc1nn2cccnc2c1C(=O)N[C@H]1CC[C@@]2(CCN(c3ccc(F)cc3Cl)C2=O)CC1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 2-methylpyrazolo[1,5-a]pyrimidine-3-carboxylic acid (73.1 mg, 413 μmol, Cas No 96319-38-9) as coupling partner. For work-up the reaction mixture was concentrated, the residue was stirred with methanol and the precipitate was collected by filtration. The thus obtained solid was purified by preparative HPLC (Method 9) to give the title compound (39 mg).

LC-MS (Method 2): Rₜ=1.09 min; MS (ESIpos): m/z=456 [M+H]⁺

¹H-NMR (500 MHz, DMSO-d6) δ[ppm]: 1.440 (0.45), 1.447 (0.44), 1.464 (0.78), 1.481 (0.55), 1.488 (0.53), 1.672 (2.07), 1.680 (2.41), 1.691 (1.78), 1.697 (1.77), 1.966 (0.77), 1.972 (0.81), 1.982 (0.51), 1.991 (0.76), 1.998 (0.70), 2.150 (1.47), 2.165 (2.70), 2.178 (1.56), 2.361 (0.42), 2.515 (1.17), 2.518 (1.14), 2.522 (0.82), 2.635 (0.47), 2.639 (0.41), 2.649 (16.00), 3.160 (0.63), 3.171 (0.61), 3.611 (1.60), 3.626 (2.73), 3.639 (1.59), 3.830 (0.41), 3.845 (0.41), 7.196 (1.94), 7.205 (1.56), 7.210 (1.78), 7.219 (1.63), 7.293 (0.68), 7.299 (0.76), 7.310 (0.98), 7.317 (1.10), 7.327 (0.79), 7.333 (0.88), 7.471 (1.41), 7.482 (1.48), 7.489 (1.26), 7.500 (1.18), 7.581 (1.42), 7.587 (1.48), 7.598 (1.40), 7.604 (1.35), 8.004 (1.20), 8.019 (1.17), 8.754 (1.88), 8.758 (1.84), 8.763 (1.88), 8.766 (1.80), 9.196 (2.07), 9.200 (1.69), 9.210 (2.03), 9.213 (1.69).

### Example 217

### N-[(trans)-2-(2-chloro-4-fluorophenyl)-1-oxo-2-azaspiro[4.5]dec-8-yl]-5-(dimethylamino)pyrazolo[1,5-a]pyrimidine-3-carboxamide

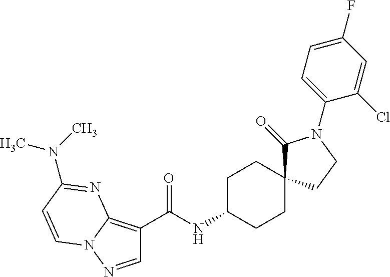 [SMILES: CN(C)c1ccn2ncc(C(=O)N[C@H]3CC[C@@]4(CCN(c5ccc(F)cc5Cl)C4=O)CC3)c2n1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide 5-(dimethylamino)pyrazolo[1,5-a]pyrimidine-3-carboxylic acid (85.1 mg, 413 μmol) as coupling partner. For work-up the reaction mixture was concentrated, the residue was stirred with methanol and the precipitate was collected by filtration. The thus obtained solid was purified by preparative HPLC (Method 9) to give the title compound (39 mg).

LC-MS (Method 2): Rₜ=1.08 min; MS (ESIpos): m/z=485 [M+H]⁺

¹H-NMR (500 MHz, DMSO-d6) δ[ppm]: 1.362 (0.41), 1.375 (0.45), 1.666 (1.41), 1.674 (1.71), 1.688 (0.89), 1.695 (0.68), 2.021 (0.51), 2.028 (0.54), 2.047 (0.51), 2.054 (0.48), 2.118 (0.97), 2.132 (1.78), 2.146 (0.99), 2.515 (0.76), 2.518 (0.66), 2.522 (0.50), 3.160 (0.86), 3.171 (0.91), 3.208 (16.00), 3.604 (1.03), 3.618 (1.80), 3.631 (1.02), 6.717 (1.80), 6.733 (1.83), 7.290 (0.42), 7.296 (0.43), 7.308 (0.64), 7.313 (0.72), 7.324 (0.50), 7.330 (0.53), 7.463 (0.90), 7.475 (0.91), 7.480 (0.81), 7.492 (0.74), 7.578 (0.86), 7.584 (0.89), 7.595 (0.92), 7.601 (0.93), 7.884 (0.81), 7.898 (0.79), 8.133 (4.04), 8.729 (2.34), 8.744 (2.32).

### Example 218

### N-[(trans)-2-(2-chloro-4-fluorophenyl)-1-oxo-2-azaspiro[4.5]dec-8-yl]-5-methoxypyrazolo[1,5-a]pyrimidine-3-carboxamide

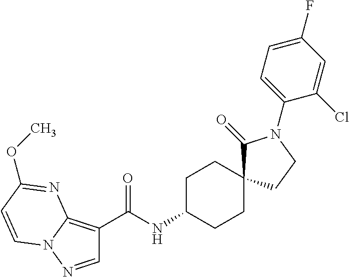 [SMILES: COc1ccn2ncc(C(=O)N[C@H]3CC[C@@]4(CCN(c5ccc(F)cc5Cl)C4=O)CC3)c2n1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide 5-methoxypyrazolo[1,5-a]pyrimidine-3-carboxylic acid (79.7 mg, 413 μmol) as coupling partner.

For work-up the reaction mixture was concentrated, the residue was stirred with methanol and the precipitate was collected by filtration and dried to give the title compound (91 mg).

LC-MS (Method 2): Rₜ=1.11 min; MS (ESIpos): m/z=472 [M+H]⁺

¹H-NMR (400 MHz, DMSO-d₆): δ [ppm]=9.06 (d, 1H), 8.36 (s, 1H), 7.66-7.57 (m, 2H), 7.48 (dd, 1H), 7.31 (td, 1H), 6.76 (d, 1H), 4.07 (s, 3H), 3.82-3.72 (m, 1H), 3.62 (t, 2H), 2.15 (t, 2H), 2.08-1.99 (m, 2H), 1.74-1.64 (m, 4H), 1.53-1.38 (m, 2H)

### Example 219

### N-[(trans)-2-(2-chloro-4-fluorophenyl)-1-oxo-2-azaspiro[4.5]dec-8-yl]-7-(methoxymethyl)-5-methylpyrazolo[1,5-a]pyrimidine-3-carboxamide

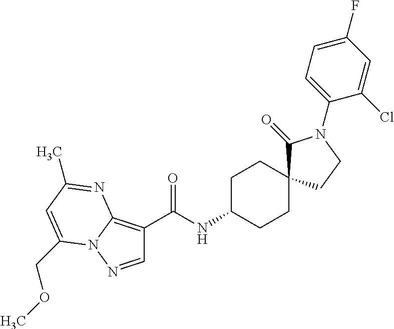 [SMILES: COCc1cc(C)nc2c(C(=O)N[C@H]3CC[C@@]4(CCN(c5ccc(F)cc5Cl)C4=O)CC3)cnn12]

Benzotriazol-1-yl-oxytripyrrolidinophosphonium hexafluorophosphate (206 mg, 396 μmol) and N,N-diisopropylethylamine (290 μl, 1.7 mmol) were added to a mixture of 8-amino-2-(2-chloro-4-fluorophenyl)-2-azaspiro[4.5]decan-1-one (isomer 1) (100 mg, 330 μmol) and a mixture of regioisomers of 5-(methoxymethyl)-7-methylpyrazolo[1,5-a]pyrimidine-3-carboxylic acid (isomer 1) and 7-(methoxymethyl)-5-methylpyrazolo[1,5-a]pyrimidine-3-carboxylic acid (isomer 2) (91.3 mg, 413 μmol) in N,N-dimethylformamide (3.7 ml) and the mixture was stirred at room temperature for 12 h. For work-up, the mixture was concentrated and the residue was purified by preparative HPLC [Instrument: Waters Autopurificationsystem SQD; column: Waters XBrigde C18 5 μm 100×30 mm; eluent A: water+0.2% Vol. ammonia (32%), eluent B: acetonitrile; gradient: 0.00-0.50 min 10% B (25->70 ml/min), 0.51-5.50 min 10-20% B (70 ml/min), temperature: Rt; DAD scan: 210-400 nm; MS ESI-Pos., scan range 160-1000 m/z] to give N-[(trans)-2-(2-chloro-4-fluorophenyl)-1-oxo-2-azaspiro[4.5]dec-8-yl]-7-(methoxymethyl)-5-methylpyrazolo[1,5-a]pyrimidine-3-carboxamide (12.1 mg Rₜ=3.9-4.3 min) and N-[(trans)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide (51.0 mg Rₜ=3.2-3.5 min).

### N-[(trans)-2-(2-chloro-4-fluorophenyl)-1-oxo-2-azaspiro[4.5]dec-8-yl]-7-(methoxymethyl)-5-methylpyrazolo[1,5-a]pyrimidine-3-carboxamide

LC-MS (Method 2): Rₜ=1.23 min; MS (ESIpos): m/z=500 [M+H]⁺

¹H-NMR (400 MHz, DMSO-d6) δ[ppm]: 1.681 (2.15), 1.689 (2.43), 1.706 (1.56), 2.155 (1.32), 2.172 (2.51), 2.189 (1.38), 2.323 (0.87), 2.327 (1.22), 2.331 (0.87), 2.523 (2.41), 2.665 (0.93), 2.669 (1.26), 2.674 (0.93), 2.695 (11.88), 3.521 (16.00), 3.614 (1.40), 3.632 (2.47), 3.648 (1.32), 4.962 (4.36), 4.965 (4.20), 7.165 (2.70), 7.310 (0.95), 7.317 (1.03), 7.339 (0.69), 7.470 (1.24), 7.485 (1.30), 7.492 (1.03), 7.507 (0.97), 7.579 (1.22), 7.587 (1.24), 7.601 (1.24), 7.608 (1.22), 7.981 (1.07), 8.000 (1.03), 8.504 (4.93).

### Example 220

### N-[(trans)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide

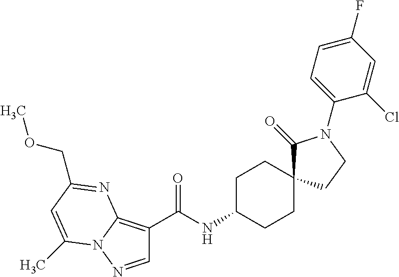 [SMILES: COCc1cc(C)n2ncc(C(=O)N[C@H]3CC[C@@]4(CCN(c5ccc(F)cc5Cl)C4=O)CC3)c2n1]

Isolated as side-product in the synthesis of N-[(trans)-2-(2-chloro-4-fluorophenyl)-1-oxo-2-azaspiro[4.5]dec-8-yl]-7-(methoxymethyl)-5-methylpyrazolo[1,5-a]pyrimidine-3-carboxamide: (51.0 mg Rₜ=3.2-3.5 min)

LC-MS (Method 2): Rₜ=1.17 min; MS (ESIpos): m/z=500.3 [M+H]⁺

¹H-NMR (400 MHz, DMSO-d6) δ[ppm]: 0.000 (1.29), 1.473 (0.49), 1.489 (0.44), 1.684 (1.69), 1.693 (1.93), 1.710 (1.27), 2.000 (0.60), 2.008 (0.64), 2.020 (0.44), 2.031 (0.60), 2.041 (0.56), 2.151 (1.06), 2.168 (1.98), 2.185 (1.13), 2.543 (0.76), 2.806 (6.01), 2.808 (6.16), 3.311 (0.85), 3.468 (16.00), 3.620 (1.13), 3.637 (1.97), 3.654 (1.09), 4.669 (6.75), 7.243 (2.01), 7.245 (1.99), 7.289 (0.50), 7.297 (0.52), 7.311 (0.76), 7.318 (0.84), 7.332 (0.58), 7.339 (0.67), 7.470 (1.07), 7.485 (1.15), 7.492 (0.94), 7.507 (0.87), 7.576 (1.10), 7.584 (1.10), 7.598 (1.08), 7.605 (1.11), 7.960 (0.85), 7.979 (0.83), 8.561 (4.95).

### Example 221

### N-[(trans)-2-(2-chloro-4-fluorophenyl)-1-oxo-2-azaspiro[4.5]dec-8-yl]-5-methylpyrazolo[1,5-a]pyrimidine-3-carboxamide

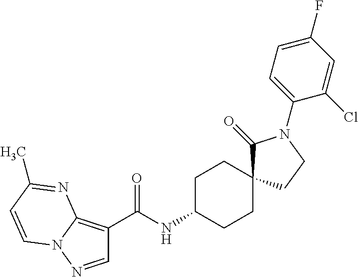 [SMILES: Cc1ccn2ncc(C(=O)N[C@H]3CC[C@@]4(CCN(c5ccc(F)cc5Cl)C4=O)CC3)c2n1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 5-methylpyrazolo[1,5-a]pyrimidine-3-carboxylic acid (74.6 mg, 421 μmol, Cas No 62908-85-4) as coupling partner. For work-up the reaction mixture was concentrated, the residue was stirred with methanol and the precipitate was collected by filtration. The thus obtained solid was purified by preparative HPLC (Method 9) to give the title compound (29 mg).

LC-MS (Method 2): Rₜ=1.09 min; MS (ESIpos): m/z=456.1 [M+H]⁺

¹H-NMR (600 MHz, DMSO-d₆): δ [ppm]=9.14 (d, 1H), 8.49-8.45 (m, 1H), 7.92 (d, 1H), 7.58 (dd, 1H), 7.48 (dd, 1H), 7.31 (td, 1H), 7.15 (d, 1H), 3.82 (tdt, 1H), 3.66-3.61 (m, 2H), 2.67 (s, 3H), 2.17 (t, 2H), 2.05-1.99 (m, 2H), 1.74-1.65 (m, 4H), 1.53-1.43 (m, 2H)

### Example 222

### N-[(trans)-2-(2-chloro-4-fluorophenyl)-1-oxo-2-azaspiro[4.5]dec-8-yl]-7-methylpyrazolo[1,5-a]pyrimidine-3-carboxamide

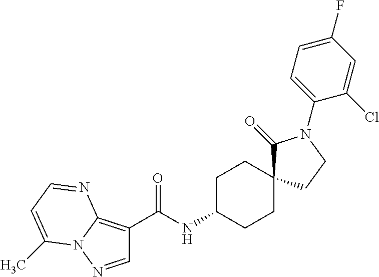 [SMILES: Cc1ccnc2c(C(=O)N[C@H]3CC[C@@]4(CCN(c5ccc(F)cc5Cl)C4=O)CC3)cnn12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 7-methylpyrazolo[1,5-a]pyrimidine-3-carboxylic acid (74.6 mg, 421 μmol, CAS No 1016505-59-1, described in WO2012007375) as coupling partner. For work-up the reaction mixture was concentrated, the residue was purified by preparative HPLC (Method 9) to give the title compound (17 mg).

LC-MS (Method 2): Rₜ=1.09 min; MS (ESIpos): m/z=456 [M+H]⁺

¹H-NMR (600 MHz, DMSO-d6) δ[ppm]: 1.474 (1.13), 1.483 (1.27), 1.492 (1.44), 1.502 (1.37), 1.512 (0.83), 1.680 (5.01), 1.686 (5.51), 1.692 (3.10), 1.697 (3.69), 1.703 (2.50), 1.975 (1.72), 1.981 (1.79), 1.989 (1.17), 1.996 (1.72), 2.003 (1.55), 2.157 (2.90), 2.169 (5.34), 2.181 (3.06), 2.381 (0.83), 2.384 (1.15), 2.387 (0.83), 2.480 (2.31), 2.484 (3.50), 2.515 (4.70), 2.518 (4.57), 2.521 (4.05), 2.526 (1.84), 2.609 (0.97), 2.612 (1.25), 2.615 (0.92), 2.807 (16.00), 3.616 (3.15), 3.628 (5.36), 3.639 (3.01), 3.833 (0.71), 3.840 (0.89), 3.846 (0.75), 3.852 (0.89), 7.232 (2.49), 7.234 (2.45), 7.240 (2.57), 7.241 (2.57), 7.292 (1.01), 7.297 (1.04), 7.306 (1.76), 7.311 (1.84), 7.320 (1.18), 7.325 (1.15), 7.468 (2.10), 7.478 (2.26), 7.483 (1.97), 7.493 (1.72), 7.574 (2.02), 7.578 (2.07), 7.588 (2.03), 7.593 (2.02), 7.897 (2.24), 7.909 (2.16), 8.592 (7.41), 8.715 (4.43), 8.722 (4.16).

### Example 223

### N-[(trans)-2-(2-chloro-4-fluorophenyl)-1-oxo-2-azaspiro[4.5]dec-8-yl]imidazo[1,2-a]pyridine-3-carboxamide

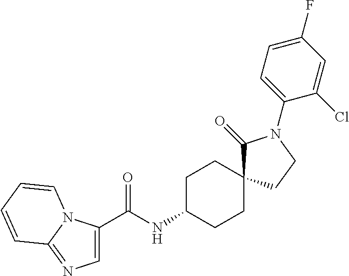 [SMILES: O=C(N[C@H]1CC[C@@]2(CCN(c3ccc(F)cc3Cl)C2=O)CC1)c1cnc2ccccn12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using imidazo[1,2-a]pyridine-3-carboxylic acid (38.4 mg, 237 μmol, Cas No 6200-60-8) as coupling partner. For work-up, the reaction mixture was concentrated and purified by preparative HPLC (Method 9) followed by recrystallization from 2-propanol to give the title compound (41 mg).

LC-MS (Method 2): Rₜ=1.04 min; MS (ESIneg): m/z=439 [M−H]⁻

¹H-NMR (400 MHz, DMSO-d6) δ[ppm]: 1.485 (1.61), 1.498 (2.09), 1.515 (2.01), 1.529 (2.29), 1.558 (1.05), 1.631 (1.07), 1.656 (3.36), 1.665 (3.45), 1.685 (6.39), 1.695 (5.71), 1.876 (2.46), 1.884 (2.60), 1.907 (2.37), 1.917 (2.09), 2.126 (4.04), 2.144 (7.55), 2.161 (4.18), 2.323 (0.73), 2.327 (1.02), 2.332 (0.71), 2.518 (3.28), 2.523 (2.26), 2.665 (0.73), 2.669 (1.02), 3.159 (1.41), 3.171 (1.55), 3.622 (4.52), 3.630 (1.30), 3.640 (7.75), 3.649 (1.27), 3.657 (4.27), 3.828 (0.96), 3.838 (1.22), 3.847 (0.99), 3.857 (1.16), 3.867 (0.90), 5.760 (1.33), 7.093 (2.15), 7.097 (2.12), 7.110 (4.38), 7.114 (4.24), 7.128 (2.35), 7.131 (2.32), 7.291 (1.87), 7.299 (2.01), 7.313 (2.97), 7.320 (3.31), 7.333 (2.20), 7.341 (2.46), 7.425 (2.60), 7.428 (2.60), 7.442 (2.32), 7.445 (2.63), 7.447 (2.97), 7.450 (2.97), 7.464 (6.53), 7.468 (3.17), 7.479 (4.47), 7.487 (3.56), 7.501 (3.28), 7.582 (4.21), 7.589 (4.16), 7.603 (4.18), 7.611 (4.01), 7.686 (3.22), 7.689 (5.43), 7.692 (3.14), 7.708 (2.57), 7.712 (4.69), 7.714 (2.57), 8.292 (3.42), 8.312 (3.34), 8.359 (16.00), 9.471 (2.91), 9.474 (4.81), 9.477 (2.94), 9.488 (2.66), 9.492 (4.49), 9.495 (2.54).

### Example 224

### N-[(trans)-2-(2-chloro-4-fluorophenyl)-1-oxo-2-azaspiro[4.5]dec-8-yl]-7-(difluoromethyl)-5-methylpyrazolo[1,5-a]pyrimidine-3-carboxamide

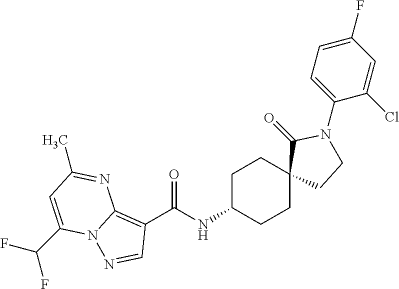 [SMILES: Cc1cc(C(F)F)n2ncc(C(=O)N[C@H]3CC[C@@]4(CCN(c5ccc(F)cc5Cl)C4=O)CC3)c2n1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazlo[1,5-a]pyrimidine-3-carboxamide using 7-(difluoromethyl)-5-methylpyrazolo[1,5-a]pyrimidine-3-carboxylic acid (95.7 mg, 421 μmol, Cas No 438218-14-5) as coupling partner. For work-up the reaction mixture was concentrated and purified by preparative HPLC (Method 9) to give the title compound (91 mg).

LC-MS (Method 2): Rₜ=1.22 min; MS (ESIpos): m/z=506 [M+H]⁺

¹H-NMR (400 MHz, DMSO-d₆): δ [ppm]=8.60 (s, 1H), 7.90 (d, 1H), 7.76-7.45 (m, 4H), 7.31 (td, 1H), 3.88-3.78 (m, 1H), 3.63 (t, 2H), 2.75 (s, 3H), 2.17 (t, 2H), 2.06-1.95 (m, 2H), 1.76-1.63 (m, 4H), 1.58-1.43 (m, 2H)

### Example 225

### tert-butyl 4-(3-{[(trans)-2-(2-chloro-4-fluorophenyl)-1-oxo-2-azaspiro[4.5]dec-8-yl]carbamoyl}pyrazolo[1,5-a]pyrimidin-5-yl)piperazine-1-carboxylate

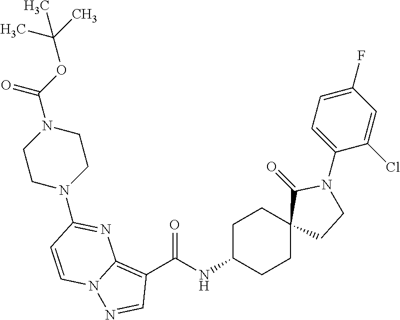 [SMILES: CC(C)(C)OC(=O)N1CCN(c2ccn3ncc(C(=O)N[C@H]4CC[C@@]5(CCN(c6ccc(F)cc6Cl)C5=O)CC4)c3n2)CC1]

Benzotriazol-1-yl-oxytripyrrolidinophosphonium hexafluorophosphate (168 mg, 323 μmol) and N,N-diisopropylethylamine (230 μl, 1.3 mmol) were added to a mixture of 8-amino-2-(2-chloro-4-fluorophenyl)-2-azaspiro[4.5]decan-1-one (isomer 1) (80.0 mg, 270 μmol) and 5-[4-(tert-butoxycarbonyl)piperazin-1-yl]pyrazolo[1,5-a]pyrimidine-3-carboxylic acid (117 mg, 337 μmol) in N,N-dimethylformamide (3.0 ml) and the mixture was stirred at room temperature for 12 h. For work-up, the mixture was concentrated, and the residue was stirred with methanol. The precipitated formed was collected by filtration, washed with water and methanol and dried at 50° C. to give the title compound (112 mg).

LC-MS (Method 2): Rₜ=1.28 min; MS (ESIpos): m/z=626.6 [M+H]⁺

¹H-NMR (400 MHz, DMSO-d6) δ[ppm]: 1.432 (16.00), 1.669 (1.03), 1.678 (1.18), 2.159 (1.25), 2.518 (1.42), 2.523 (1.10), 3.502 (1.18), 3.518 (1.03), 3.633 (1.21), 3.762 (1.19), 6.860 (0.90), 6.880 (0.93), 7.468 (0.69), 7.483 (0.72), 7.579 (0.70), 7.587 (0.69), 7.601 (0.70), 7.608 (0.69), 8.172 (3.23), 8.801 (1.42), 8.821 (1.36).

### Example 226

### N-[(trans)-2-(2-chloro-4-fluorophenyl)-1-oxo-2-azaspiro[4.5]dec-8-yl]-5-(piperazin-1-yl)pyrazolo[1,5-a]pyrimidine-3-carboxamide hydrochloric acid salt

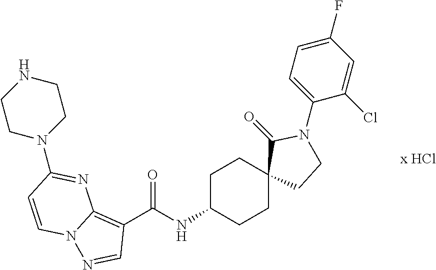 [SMILES: O=C(N[C@H]1CC[C@@]2(CCN(c3ccc(F)cc3Cl)C2=O)CC1)c1cnn2ccc(N3CCNCC3)nc12]

Hydrochloric acid (330 μl, 4.0 M in dioxane, 1.3 mmol) was added to a suspension of tert-butyl 4-(3-{[2-(2-chloro-4-fluorophenyl)-1-oxo-2-azaspiro[4.5]dec-8-yl]carbamoyl}pyrazolo[1,5-a]pyrimidin-5-yl)piperazine-1-carboxylate (isomer 1) (82.0 mg, 131 μmol) in dichloromethane (1.4 ml) and the mixture was stirred at room temperature overnight. The precipitate was collected by filtration and dried to give the title compound (73 mg).

LC-MS (Method 2): Rₜ=0.93 min; MS (ESIpos): m/z=526.6 [M+H]⁺

¹H-NMR (400 MHz, DMSO-d6) δ[ppm]: 1.390 (1.11), 1.404 (1.26), 1.420 (1.45), 1.433 (1.30), 1.447 (0.76), 1.463 (0.57), 1.664 (4.66), 1.673 (5.38), 1.690 (3.13), 1.698 (2.33), 1.907 (0.80), 1.995 (1.72), 2.003 (1.79), 2.026 (1.72), 2.034 (1.53), 2.133 (2.90), 2.151 (5.54), 2.168 (3.05), 2.323 (0.76), 2.327 (1.07), 2.332 (0.76), 2.518 (2.98), 2.523 (2.02), 2.665 (0.76), 2.669 (1.07), 2.673 (0.73), 3.254 (4.01), 3.266 (5.58), 3.278 (4.32), 3.385 (0.42), 3.565 (0.42), 3.608 (3.13), 3.626 (5.42), 3.642 (2.98), 3.710 (0.69), 3.720 (0.84), 3.729 (0.73), 3.738 (0.88), 3.748 (0.65), 3.956 (4.20), 3.969 (5.58), 3.981 (3.93), 6.929 (4.12), 6.948 (4.20), 7.294 (1.45), 7.301 (1.53), 7.315 (2.14), 7.322 (2.41), 7.336 (1.72), 7.343 (1.79), 7.464 (2.98), 7.478 (3.25), 7.486 (2.60), 7.501 (2.41), 7.583 (3.09), 7.591 (3.28), 7.605 (3.25), 7.612 (3.28), 7.626 (2.60), 7.644 (2.41), 8.218 (16.00), 8.231 (0.42), 8.881 (6.45), 8.900 (6.00), 9.190 (0.61).

### Example 227

### N-[(trans)-2-(2-chloro-4-fluorophenyl)-1-oxo-2-azaspiro[4.5]dec-8-yl]-2,5,7-trimethylpyrazolo[1,5-a]pyrimidine-3-carboxamide

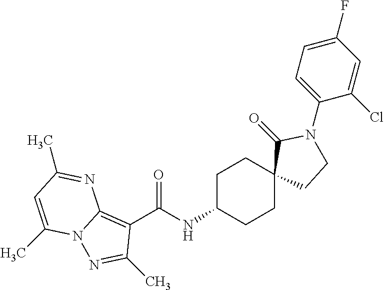 [SMILES: Cc1cc(C)n2nc(C)c(C(=O)N[C@H]3CC[C@@]4(CCN(c5ccc(F)cc5Cl)C4=O)CC3)c2n1]

Benzotriazol-1-yl-oxytripyrrolidinophosphonium hexafluorophosphate (168 mg, 323 μmol) and N,N-diisopropylethylamine (230 μl, 1.3 mmol) were added to a mixture of 8-amino-2-(2-chloro-4-fluorophenyl)-2-azaspiro[4.5]decan-1-one (isomer 1) (80.0 mg, 270 μmol) and 2,5,7-trimethylpyrazolo[1,5-a]pyrimidine-3-carboxylic acid (69.1 mg, 337 μmol, Cas No 1158269-53-4) in N,N-dimethylformamide (3.0 ml) and the mixture was stirred at room temperature for 12 h. For work-up, the mixture was concentrated, and the residue was stirred with methanol. The precipitated formed was collected by filtration and purified by flash chromatography (ethyl acetate/methanol gradient, 0%->30% methanol) to give the title compound (65 mg).

LC-MS (Method 2): Rₜ=1.24 min; MS (ESIneg): m/z=482 [M−H]⁻

¹H-NMR (400 MHz, DMSO-d6) δ[ppm]: 1.434 (0.50), 1.456 (0.71), 1.477 (0.61), 1.678 (2.39), 1.687 (2.74), 1.703 (1.78), 1.999 (0.88), 2.008 (0.92), 2.019 (0.61), 2.031 (0.86), 2.040 (0.79), 2.149 (1.51), 2.166 (2.85), 2.183 (1.57), 2.327 (0.42), 2.518 (1.45), 2.523 (0.92), 2.605 (14.41), 2.632 (16.00), 2.645 (0.53), 2.658 (0.46), 2.669 (0.47), 2.698 (8.44), 2.700 (8.86), 3.611 (1.60), 3.629 (2.82), 3.646 (1.53), 3.800 (0.43), 3.818 (0.42), 7.053 (3.02), 7.055 (3.05), 7.288 (0.66), 7.295 (0.76), 7.310 (1.08), 7.317 (1.18), 7.331 (0.80), 7.338 (0.91), 7.470 (1.48), 7.485 (1.58), 7.492 (1.29), 7.507 (1.21), 7.578 (1.53), 7.585 (1.58), 7.600 (1.57), 7.607 (1.53), 8.252 (1.24), 8.271 (1.20).

### Example 228

### N-[(trans)-2-(2-chloro-4-fluorophenyl)-1-oxo-2-azaspiro[4.5]dec-8-yl]-1H-pyrazolo[4,3-b]pyridine-3-carboxamide

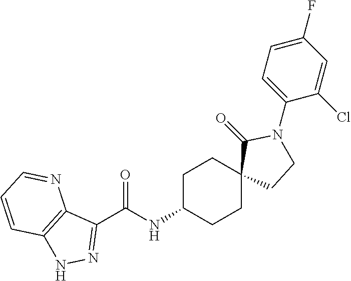 [SMILES: O=C(N[C@H]1CC[C@@]2(CCN(c3ccc(F)cc3Cl)C2=O)CC1)c1n[nH]c2cccnc12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazlo[1,5-a]pyrimidine-3-carboxamide using 1H-pyrazolo[4,3-b]pyridine-3-carboxylic acid (55.0 mg, 337 μmol, Cas No 1260648-73-4) as coupling partner. For work-up the reaction mixture was concentrated and the residue and purified by preparative HPLC (Method 9) to give the title compound (9.0 mg).

LC-MS (Method 2): Rₜ=0.98 min; MS (ESIneg): m/z=440 [M−H]⁻

¹H-NMR (400 MHz, DMSO-d6) δ[ppm]: 1.232 (0.82), 1.489 (1.15), 1.520 (2.67), 1.543 (4.35), 1.561 (3.77), 1.592 (1.97), 1.691 (14.24), 1.700 (16.00), 1.717 (10.13), 1.897 (0.57), 1.995 (5.13), 2.004 (5.37), 2.027 (4.92), 2.036 (4.43), 2.074 (4.35), 2.160 (8.04), 2.177 (15.34), 2.194 (8.41), 2.318 (0.90), 2.323 (1.81), 2.327 (2.42), 2.331 (1.72), 2.337 (0.86), 2.523 (5.46), 2.660 (0.82), 2.665 (1.68), 2.669 (2.38), 2.674 (1.64), 2.678 (0.74), 3.616 (8.62), 3.633 (15.14), 3.650 (8.25), 3.868 (1.03), 3.887 (1.93), 3.898 (2.42), 3.906 (2.05), 3.916 (2.38), 3.926 (1.85), 3.945 (0.90), 5.759 (0.70), 7.290 (3.12), 7.298 (3.57), 7.312 (5.70), 7.319 (6.15), 7.333 (3.90), 7.340 (4.23), 7.472 (7.51), 7.481 (7.79), 7.486 (8.78), 7.493 (10.99), 7.503 (7.71), 7.509 (6.85), 7.514 (7.55), 7.581 (7.38), 7.588 (7.30), 7.602 (7.38), 7.610 (7.18), 8.163 (5.91), 8.166 (5.91), 8.185 (5.70), 8.629 (4.06), 8.648 (3.98), 8.682 (8.53), 8.685 (8.70), 8.693 (8.33), 8.696 (7.71).

### Example 229

### N-[(trans)-2-(2-chloro-4-fluorophenyl)-1-oxo-2-azaspiro[4.5]dec-8-yl]-5-(4-methylpiperazin-1-yl)pyrazolo[1,5-a]pyrimidine-3-carboxamide

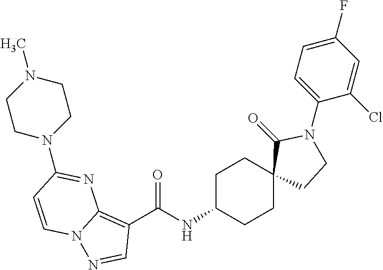 [SMILES: CN1CCN(c2ccn3ncc(C(=O)N[C@H]4CC[C@@]5(CCN(c6ccc(F)cc6Cl)C5=O)CC4)c3n2)CC1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using sodium 5-(4-methylpiperazin-1-yl)pyrazolo[1,5-a]pyrimidine-3-carboxylate (127 mg, 337 μmol) as coupling partner. For work-up the reaction mixture was concentrated and purified by preparative HPLC to give the title compound (24.0 mg).

LC-MS (Method 2): Rₜ=1.05 min; MS (ESIpos): m/z=540 [M+H]⁺

¹H-NMR (400 MHz, DMSO-d6) δ[ppm]: 1.347 (1.13), 1.363 (1.27), 1.379 (1.44), 1.391 (1.25), 1.406 (0.72), 1.662 (4.48), 1.671 (5.39), 1.688 (2.93), 1.697 (2.19), 1.825 (0.76), 2.003 (1.68), 2.012 (1.77), 2.036 (1.68), 2.043 (1.54), 2.120 (2.58), 2.137 (4.95), 2.154 (2.72), 2.241 (16.00), 2.304 (0.60), 2.323 (0.70), 2.327 (0.93), 2.331 (0.67), 2.442 (4.17), 2.454 (6.05), 2.467 (4.60), 2.523 (2.28), 2.665 (0.66), 2.669 (0.89), 2.673 (0.64), 3.609 (2.83), 3.626 (4.94), 3.643 (2.66), 3.725 (4.66), 3.738 (5.93), 3.750 (4.28), 6.882 (3.39), 6.901 (3.45), 7.287 (1.02), 7.294 (1.12), 7.308 (1.83), 7.316 (1.97), 7.330 (1.25), 7.337 (1.30), 7.462 (2.28), 7.477 (2.45), 7.485 (1.97), 7.499 (1.80), 7.578 (2.29), 7.585 (2.34), 7.599 (2.34), 7.606 (2.25), 7.770 (2.29), 7.788 (2.22), 8.156 (9.57), 8.758 (4.37), 8.779 (4.26).

### Example 230

### N-[(trans)-2-(2-chloro-4-fluorophenyl)-1-oxo-2-azaspiro[4.5]dec-8-yl]pyrazolo[1,5-a]pyridine-3-carboxamide

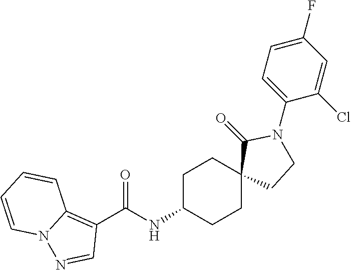 [SMILES: O=C(N[C@H]1CC[C@@]2(CCN(c3ccc(F)cc3Cl)C2=O)CC1)c1cnn2ccccc12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using pyrazolo[1,5-a]pyridine-3-carboxylic acid (36.5 mg, 225 μmol, Cas No 16205-46-2) as coupling partner. For work-up the reaction mixture was concentrated and purified by preparative HPLC (Method 9), followed by recrystallization from acetonitrile to give the title compound (32.0 mg).

LC-MS (Method 2): Rₜ=1.05 min; MS (ESIneg): m/z=439 [M−H]⁻

¹H-NMR (400 MHz, DMSO-d6) δ[ppm]: 1.459 (1.77), 1.471 (2.26), 1.489 (2.15), 1.502 (2.46), 1.532 (1.11), 1.620 (1.15), 1.644 (3.54), 1.654 (3.47), 1.674 (6.80), 1.684 (5.97), 1.864 (2.60), 1.873 (2.74), 1.896 (2.50), 1.905 (2.22), 2.075 (1.39), 2.123 (4.37), 2.140 (8.12), 2.158 (4.55), 2.322 (0.80), 2.327 (1.11), 2.332 (0.80), 2.518 (3.23), 2.523 (2.39), 2.665 (0.80), 2.669 (1.11), 2.673 (0.76), 3.620 (4.86), 3.628 (1.35), 3.637 (8.36), 3.655 (4.72), 3.795 (1.01), 3.805 (1.28), 3.814 (1.08), 3.824 (1.25), 7.026 (2.43), 7.030 (2.33), 7.043 (4.65), 7.047 (4.55), 7.060 (2.64), 7.064 (2.60), 7.290 (2.12), 7.297 (2.33), 7.311 (3.19), 7.319 (3.64), 7.333 (2.46), 7.340 (2.88), 7.426 (2.78), 7.429 (2.98), 7.443 (2.92), 7.446 (3.02), 7.449 (3.26), 7.451 (3.33), 7.463 (5.24), 7.468 (3.12), 7.478 (5.00), 7.485 (3.99), 7.500 (3.71), 7.581 (4.72), 7.588 (4.86), 7.603 (4.82), 7.610 (4.65), 8.008 (3.68), 8.028 (3.57), 8.197 (3.05), 8.200 (4.93), 8.203 (3.19), 8.219 (2.78), 8.222 (4.41), 8.226 (2.74), 8.574 (16.00), 8.744 (3.37), 8.747 (6.39), 8.749 (3.44), 8.761 (3.47), 8.764 (6.11), 8.766 (3.19).

### Example 231

### N-[(trans)-2-(2-chloro-4-fluorophenyl)-1-oxo-2-azaspiro[4.5]dec-8-yl]-5-(morpholin-4-yl)pyrazolo[1,5-a]pyrimidine-3-carboxamide

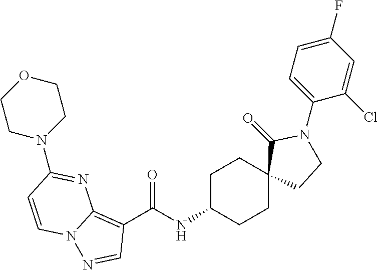 [SMILES: O=C(N[C@H]1CC[C@@]2(CCN(c3ccc(F)cc3Cl)C2=O)CC1)c1cnn2ccc(N3CCOCC3)nc12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 5-(morpholin-4-yl)pyrazolo[1,5-a]pyrimidine-3-carboxylic acid (83.6 mg, 337 μmol) as coupling partner. For work-up the reaction mixture was concentrated and purified by preparative HPLC (Method 9), followed by recrystallization from acetonitrile to give the title compound (58.0 mg).

LC-MS (Method 2): Rₜ=1.06 min; MS (ESIpos): m/z=527 [M+H]⁺

¹H-NMR (400 MHz, DMSO-d6) δ[ppm]: 1.358 (1.37), 1.372 (1.54), 1.388 (1.74), 1.401 (1.54), 1.657 (5.47), 1.666 (6.53), 1.683 (3.73), 1.692 (2.70), 1.993 (2.02), 2.003 (2.12), 2.014 (1.40), 2.026 (2.02), 2.035 (1.81), 2.119 (3.38), 2.137 (6.46), 2.154 (3.56), 2.518 (3.25), 2.523 (2.05), 2.669 (0.89), 3.604 (3.69), 3.622 (6.39), 3.639 (3.49), 3.722 (7.21), 3.730 (10.32), 3.748 (10.36), 3.755 (7.01), 3.759 (6.53), 6.875 (5.06), 6.894 (4.99), 7.286 (1.57), 7.294 (1.61), 7.308 (2.46), 7.315 (2.70), 7.328 (1.81), 7.336 (1.95), 7.461 (3.42), 7.476 (3.62), 7.483 (2.91), 7.498 (2.70), 7.577 (3.35), 7.584 (3.52), 7.598 (3.49), 7.605 (3.42), 7.721 (3.01), 7.739 (2.87), 8.175 (16.00), 8.798 (7.18), 8.817 (6.91).

### Example 232

### N-[(trans)-2-(2-chloro-4-fluorophenyl)-1-oxo-2-azaspiro[4.5]dec-8-yl]-5-(pyrrolidin-1-yl)pyrazolo[1,5-a]pyrimidine-3-carboxamide

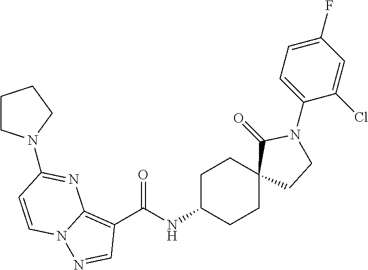 [SMILES: O=C(N[C@H]1CC[C@@]2(CCN(c3ccc(F)cc3Cl)C2=O)CC1)c1cnn2ccc(N3CCCC3)nc12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 5-(pyrrolidin-1-yl)pyrazolo[1,5-a]pyrimidine-3-carboxylic acid (78.3 mg, 337 μmol) as coupling partner. For work-up the reaction mixture was concentrated and the residue was stirred with methanol. The precipitate formed was collected by filtration and dried to give the title compound (78.0 mg).

LC-MS (Method 2): Rₜ=1.20 min; MS (ESIpos): m/z=511 [M+H]⁺

¹H-NMR (600 MHz, DMSO-d6) δ[ppm]: 1.340 (1.17), 1.349 (1.39), 1.360 (3.35), 1.369 (3.61), 1.380 (3.95), 1.388 (3.69), 1.399 (1.96), 1.408 (1.69), 1.683 (14.38), 1.697 (6.02), 1.702 (5.87), 1.719 (1.66), 2.024 (10.84), 2.030 (11.07), 2.052 (6.10), 2.057 (5.35), 2.128 (6.02), 2.139 (11.29), 2.151 (6.48), 2.384 (1.28), 2.460 (1.02), 2.514 (16.00), 2.517 (14.68), 2.612 (1.54), 3.279 (1.05), 3.292 (2.90), 3.330 (11.71), 3.352 (2.48), 3.542 (5.95), 3.599 (6.06), 3.614 (8.73), 3.625 (12.24), 3.637 (6.81), 3.727 (1.77), 3.733 (2.15), 3.739 (1.92), 3.745 (2.18), 3.751 (1.81), 6.518 (7.12), 6.531 (7.15), 7.291 (1.77), 7.295 (1.96), 7.305 (3.73), 7.309 (3.92), 7.319 (2.33), 7.324 (2.33), 7.463 (3.76), 7.473 (4.18), 7.478 (3.69), 7.488 (3.31), 7.571 (3.73), 7.576 (3.80), 7.586 (3.92), 7.590 (3.76), 7.945 (5.08), 7.957 (5.08), 8.117 (12.27), 8.704 (6.89), 8.717 (7.12).

### Example 233

### N-[(trans)-2-(2-chloro-4-fluorophenyl)-1-oxo-2-azaspiro[4.5]dec-8-yl]-7-(trifluoromethyl)pyrazolo[1,5-a]pyrimidine-3-carboxamide

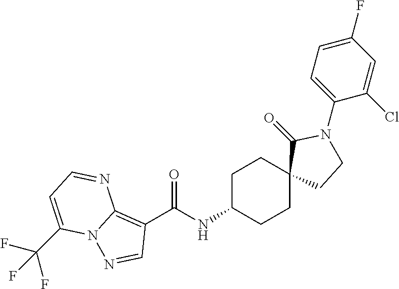 [SMILES: O=C(N[C@H]1CC[C@@]2(CCN(c3ccc(F)cc3Cl)C2=O)CC1)c1cnn2c(C(F)(F)F)ccnc12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazlo[1,5-a]pyrimidine-3-carboxamide using and 7-(trifluoromethyl)pyrazolo[1,5-a]pyrimidine-3-carboxylic acid (77.9 mg, 337 μmol, Cas No 869947-41-1) as coupling partner. For work-up the reaction mixture was concentrated and the residue was stirred with methanol. The precipitate formed was collected by filtration and dried to give the title compound (31.0 mg).

LC-MS (Method 2): Rₜ=1.18 min; MS (ESIneg): m/z=508 [M−H]⁻

¹H-NMR (400 MHz, DMSO-d6) δ[ppm]: 1.484 (1.28), 1.514 (2.25), 1.534 (1.78), 1.675 (6.51), 1.684 (7.52), 1.706 (4.92), 1.951 (2.40), 1.960 (2.48), 1.983 (2.32), 1.991 (2.09), 2.149 (3.99), 2.166 (7.63), 2.183 (4.26), 2.323 (0.93), 2.327 (1.32), 2.331 (0.97), 2.518 (4.69), 2.523 (2.94), 2.665 (0.93), 2.669 (1.28), 2.673 (0.93), 3.612 (4.22), 3.630 (7.52), 3.647 (4.11), 3.856 (1.20), 3.876 (1.16), 7.289 (1.70), 7.296 (1.78), 7.310 (2.79), 7.318 (3.14), 7.331 (2.05), 7.339 (2.21), 7.466 (3.80), 7.480 (4.03), 7.488 (3.33), 7.502 (3.06), 7.579 (3.84), 7.586 (3.87), 7.600 (3.87), 7.608 (3.72), 7.829 (3.84), 7.835 (6.35), 7.847 (7.86), 8.753 (16.00), 9.017 (5.66), 9.028 (5.31).

### Example 234

### N-[(trans)-2-(2-chloro-4-fluorophenyl)-1-oxo-2-azaspiro[4.5]dec-8-yl]-5-hydroxypyrazolo[1,5-a]pyrimidine-3-carboxamide

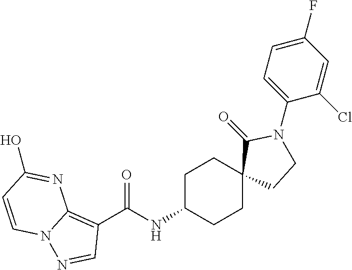 [SMILES: O=C(N[C@H]1CC[C@@]2(CCN(c3ccc(F)cc3Cl)C2=O)CC1)c1cnn2ccc(O)nc12]

A mixture of (mu-1,4-diazabicyclo[2.2.2]octane-kappaN¹:kappaN⁴) (hexamethyl) dialuminium (124 mg, 483 μmol, Cas No 137203-34-0) and 8-amino-2-(2-chloro-4-fluorophenyl)-2-azaspiro[4.5]decan-1-one (isomer 1) (143 mg, 483 μmol) in tetrahydrofuran (8.2 ml) was stirred in a sealed tube at 40° C. for 1 h followed by 1 h at room temperature. ethyl 5-oxo-4,5-dihydropyrazolo[1,5-a]pyrimidine-3-carboxylate (100 mg, 483 μmol, Cas No 926663-C₀-5) was added and the mixture was stirred for 1 d at room temperature. For work-up, the mixture was poured into ice-water, extracted with dichloromethane (3×) and the combined organic phases where filtrated through a silicone filter and concentrated. The residue was purified by preparative HPLC (Method 9) to give the title compound (7.8 mg).

LC-MS (Method 2): Rₜ=0.61 min; MS (ESIpos): m/z=458 [M+H]⁺

¹H-NMR (400 MHz, DMSO-d6) δ[ppm]: 1.232 (0.70), 1.417 (1.25), 1.429 (1.48), 1.448 (1.40), 1.460 (1.64), 1.610 (0.86), 1.634 (2.26), 1.644 (2.19), 1.665 (4.53), 1.862 (1.80), 1.886 (1.64), 1.907 (2.03), 2.115 (2.89), 2.133 (5.39), 2.150 (2.97), 2.318 (1.40), 2.322 (3.20), 2.327 (4.53), 2.332 (3.28), 2.336 (1.48), 2.518 (16.00), 2.523 (10.07), 2.539 (5.46), 2.660 (1.48), 2.664 (3.20), 2.669 (4.60), 2.673 (3.36), 2.678 (1.48), 3.613 (3.28), 3.631 (5.54), 3.648 (3.04), 3.753 (0.86), 3.772 (0.86), 6.094 (0.78), 7.288 (1.48), 7.295 (1.56), 7.309 (2.26), 7.317 (2.50), 7.330 (1.72), 7.337 (1.87), 7.460 (3.12), 7.475 (3.28), 7.482 (2.65), 7.497 (2.34), 7.578 (3.12), 7.586 (3.12), 7.601 (3.12), 7.608 (3.12), 8.090 (0.86), 8.233 (1.48), 8.516 (0.70).

### Example 235

### N-[(trans)-2-(2-chloro-4-fluorophenyl)-1-oxo-2-azaspiro[4.5]dec-8-yl]-7-hydroxypyrazolo[1,5-a]pyrimidine-3-carboxamide

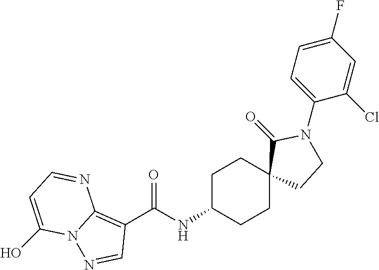 [SMILES: O=C(N[C@H]1CC[C@@]2(CCN(c3ccc(F)cc3Cl)C2=O)CC1)c1cnn2c(O)ccnc12]

Was prepared in analogy to the synthesis of N-[(trans)-2-(2-chloro-4-fluorophenyl)-1-oxo-2-azaspiro[4.5]dec-8-yl]-5-oxo-4,5-dihydropyrazolo[1,5-a]pyrimidine-3-carboxamide using and ethyl 7-oxo-4,7-dihydropyrazolo[1,5-a]pyrimidine-3-carboxylate (100 mg, 483 μmol, Cas No 104556-86-7) as coupling partner to give the title compound (6.0 mg).

LC-MS (Method 2): Rₜ=0.71 min; MS (ESIpos): m/z=458 [M+H]⁺

¹H-NMR (400 MHz, DMSO-d₆): δ [ppm]=12.11 (br d, 1H), 8.41 (s, 1H), 8.22 (br d, 1H), 7.79-7.74 (m, 1H), 7.61-7.57 (m, 1H), 7.48 (dd, 1H), 7.34-7.29 (m, 1H), 5.85 (br d, 1H), 3.86-3.73 (m, 1H), 3.64 (t, 2H), 2.14 (t, 2H), 1.96-1.82 (m, 2H), 1.75-1.60 (m, 4H), 1.54-1.40 (m, 2H)

### Example 236

### N-[(trans)-2-(2-chloro-4-fluorophenyl)-1-oxo-2-azaspiro[4.5]dec-8-yl]-1H-pyrrolo[3,2-b]pyridine-3-carboxamide

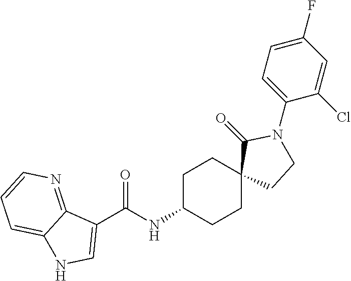 [SMILES: O=C(N[C@H]1CC[C@@]2(CCN(c3ccc(F)cc3Cl)C2=O)CC1)c1c[nH]c2cccnc12]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 1H-pyrrolo[3,2-b]pyridine-3-carboxylic acid (301 mg, 1.85 mmol, Cas No 860496-20-4) as coupling partner. For work-up, the reaction mixture stirred with water and the precipitate formed was collected by filtration, washed with water and methanol to give the title compound (324 mg).

LC-MS (Method 2): Rₜ=1.14 min; MS (ESIneg): m/z=439 [M−H]⁻

¹H-NMR (400 MHz, DMSO-d6) δ[ppm]: 1.447 (1.09), 1.455 (1.19), 1.467 (1.34), 1.477 (1.90), 1.496 (1.54), 1.682 (6.04), 1.691 (6.89), 1.707 (4.55), 1.713 (4.09), 1.997 (2.19), 2.005 (2.28), 2.016 (1.47), 2.028 (2.12), 2.036 (1.96), 2.164 (3.79), 2.181 (7.10), 2.198 (4.00), 2.327 (0.86), 2.518 (2.95), 2.523 (1.87), 2.669 (0.88), 3.612 (4.09), 3.630 (7.10), 3.647 (3.93), 3.841 (1.07), 3.860 (1.07), 7.236 (3.90), 7.248 (3.82), 7.257 (3.99), 7.268 (4.02), 7.288 (1.78), 7.295 (1.81), 7.310 (2.66), 7.317 (3.08), 7.330 (2.07), 7.338 (2.22), 7.470 (3.73), 7.485 (3.88), 7.492 (3.16), 7.506 (2.93), 7.578 (3.87), 7.585 (3.91), 7.600 (3.90), 7.607 (3.70), 7.906 (4.93), 7.909 (4.96), 7.926 (4.52), 7.930 (4.52), 8.166 (16.00), 8.474 (4.55), 8.478 (4.61), 8.486 (4.52), 8.489 (4.15), 8.715 (3.23), 8.734 (3.11).

### Example 237

### N-[(trans)-2-(2-chloro-4-fluorophenyl)-1-oxo-2-azaspiro[4.5]dec-8-yl]-6-(2-methoxyethyl)pyrazolo[1,5-a]pyrimidine-3-carboxamide

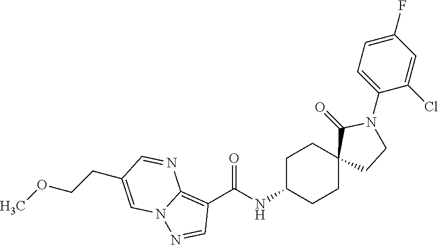 [SMILES: COCCc1cnc2c(C(=O)N[C@H]3CC[C@@]4(CCN(c5ccc(F)cc5Cl)C4=O)CC3)cnn2c1]

Was prepared in analogy to the synthesis of N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide using 6-(2-methoxyethyl)pyrazolo[1,5-a]pyrimidine-3-carboxylic acid (91.3 mg, 413 μmol) as coupling partner. For work-up the reaction mixture was concentrated and purified by preparative HPLC [Instrument: Waters Autopurificationsystem; column: Waters XBrigde C18 5p 100×30 mm; eluent A: water+0.1 Vol-% formic acid (99%), eluent B: acetonitrile; gradient: 0.00-0.50 min 24% B (25->70 mL/min), 0.51-5.50 min 24-55% B (70 ml/min), DAD scan: 210-400 nm] to give the title compound (10.5 mg).

LC-MS (Method 2): Rₜ=1.13 min; MS (ESIpos): m/z=500 [M+H]⁺

¹H-NMR (400 MHz, DMSO-d6) δ[ppm]: 1.107 (2.06), 1.459 (0.41), 1.484 (0.68), 1.503 (0.54), 1.669 (1.96), 1.677 (2.20), 1.693 (1.52), 1.962 (0.74), 1.986 (0.71), 2.147 (1.15), 2.165 (2.16), 2.182 (1.22), 2.323 (1.42), 2.327 (2.00), 2.332 (1.42), 2.518 (7.48), 2.523 (4.67), 2.665 (1.45), 2.669 (2.00), 2.673 (1.42), 2.935 (0.95), 2.950 (2.06), 2.966 (1.05), 3.263 (16.00), 3.607 (2.20), 3.622 (3.79), 3.638 (1.69), 3.643 (1.39), 7.289 (0.47), 7.296 (0.58), 7.311 (0.85), 7.317 (0.91), 7.331 (0.61), 7.338 (0.61), 7.466 (1.12), 7.481 (1.18), 7.489 (0.98), 7.503 (0.85), 7.580 (1.12), 7.587 (1.12), 7.601 (1.12), 7.609 (1.08), 7.780 (1.01), 7.800 (0.98), 8.518 (4.74), 8.773 (2.16), 8.779 (2.20), 9.196 (1.79), 9.201 (1.73).

### Example 238

### N-[(trans)-1-oxo-2-phenyl-2-azaspiro[4.5]dec-8-yl]pyrazolo[1,5-a]pyrimidine-3-carboxamide

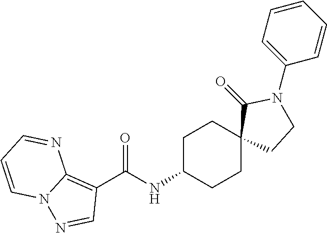 [SMILES: O=C(N[C@H]1CC[C@@]2(CCN(c3ccccc3)C2=O)CC1)c1cnn2cccnc12]

Benzotriazol-1-yl-oxytripyrrolidinophosphonium hexafluorophosphate (179 mg, 344 μmol) and N,N-diisopropylethylamine (250 μl, 1.4 mmol) were added to a mixture of 8-amino-2-phenyl-2-azaspiro[4.5]decan-1-one (isomer 1) (70.0 mg, 286 mol) and pyrazolo[1,5-a]pyrimidine-3-carboxylic acid (58.4 mg, 358 μmol, Cas No 25940-35-6) in N,N-dimethylformamide (3.2 ml) and the mixture was stirred at room temperature overnight. For work-up, the mixture was concentrated, and the residue was stirred with methanol. The precipitated formed was collected by filtration, washed with water and methanol and dried to give the title compound (69 mg).

LC-MS (Method 2): Rₜ=1.02 min; MS (ESIneg): m/z=388 [M−H]⁻

¹H-NMR (400 MHz, DMSO-d6) δ[ppm]: 1.430 (0.53), 1.440 (0.71), 1.461 (1.86), 1.471 (1.94), 1.492 (2.03), 1.501 (2.12), 1.522 (1.06), 1.532 (0.88), 1.620 (1.24), 1.655 (5.39), 1.665 (4.15), 1.690 (2.39), 1.698 (2.65), 1.723 (0.71), 1.731 (0.71), 1.943 (2.21), 1.952 (2.30), 1.963 (1.41), 1.976 (2.12), 1.984 (1.94), 2.085 (3.80), 2.102 (6.81), 2.119 (3.98), 2.322 (0.62), 2.327 (0.88), 2.332 (0.62), 2.518 (2.83), 2.523 (1.94), 2.665 (0.62), 2.669 (0.88), 2.673 (0.62), 2.727 (1.86), 2.888 (2.21), 3.299 (0.44), 3.306 (0.71), 3.309 (0.71), 3.376 (0.88), 3.385 (0.62), 3.783 (4.24), 3.800 (7.25), 3.817 (4.51), 3.834 (1.15), 3.844 (0.88), 3.854 (1.15), 3.864 (0.88), 3.873 (0.44), 3.882 (0.44), 7.111 (1.06), 7.114 (1.94), 7.117 (1.15), 7.133 (4.24), 7.148 (1.50), 7.151 (2.56), 7.154 (1.50), 7.260 (4.07), 7.271 (3.80), 7.278 (3.80), 7.288 (4.07), 7.351 (0.71), 7.357 (5.04), 7.361 (1.86), 7.375 (5.66), 7.378 (6.45), 7.392 (1.59), 7.397 (4.86), 7.682 (6.28), 7.685 (7.34), 7.704 (6.36), 7.707 (5.04), 7.817 (3.09), 7.836 (3.01), 8.580 (16.00), 8.820 (4.69), 8.824 (4.95), 8.830 (4.51), 8.834 (4.51), 9.307 (4.77), 9.312 (5.04), 9.325 (5.04), 9.330 (4.51).

### Example 239

### 5,7-dimethyl-N-[(trans)-1-oxo-2-phenyl-2-azaspiro[4.5]dec-8-yl]pyrazolo[1,5-a]pyrimidine-3-carboxamide

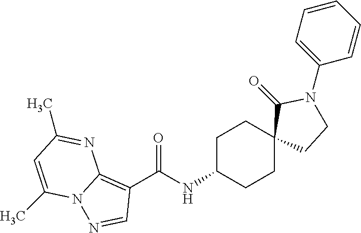 [SMILES: Cc1cc(C)n2ncc(C(=O)N[C@H]3CC[C@@]4(CCN(c5ccccc5)C4=O)CC3)c2n1]

Was prepared in analogy to the synthesis of N-[(trans)-1-oxo-2-phenyl-2-azaspiro[4.5]dec-8-yl]pyrazolo[1,5-a]pyrimidine-3-carboxamide using 5,7-dimethylpyrazolo[1,5-a]pyrimidine-3-carboxylic acid (68.5 mg, 358 μmol, Cas No 90349-23-8) as coupling partner to give the title compound (106 mg).

LC-MS (Method 2): Rₜ=1.15 min; MS (ESIneg): m/z=416 [M−H]⁻

¹H-NMR (400 MHz, DMSO-d6) δ[ppm]: 1.443 (0.74), 1.453 (0.74), 1.473 (0.80), 1.482 (0.85), 1.504 (0.43), 1.625 (0.53), 1.659 (1.86), 1.664 (1.81), 1.672 (1.44), 1.698 (0.96), 1.705 (1.06), 1.985 (0.85), 1.993 (0.90), 2.005 (0.58), 2.017 (0.85), 2.026 (0.74), 2.090 (1.54), 2.107 (2.76), 2.125 (1.65), 2.518 (1.12), 2.523 (0.80), 2.632 (16.00), 2.669 (0.43), 2.728 (1.44), 2.734 (8.61), 2.737 (9.14), 2.887 (1.01), 3.316 (0.43), 3.321 (0.43), 3.325 (0.48), 3.375 (1.12), 3.384 (0.64), 3.386 (0.69), 3.390 (0.58), 3.394 (0.80), 3.785 (1.91), 3.803 (3.14), 3.820 (1.91), 7.111 (0.48), 7.114 (0.96), 7.117 (0.69), 7.122 (3.03), 7.124 (3.08), 7.133 (1.97), 7.148 (0.64), 7.151 (1.17), 7.154 (0.64), 7.357 (2.23), 7.361 (0.74), 7.375 (2.34), 7.378 (2.76), 7.392 (0.64), 7.397 (2.13), 7.683 (2.60), 7.686 (3.08), 7.700 (0.74), 7.705 (2.76), 7.708 (2.07), 8.013 (1.28), 8.031 (1.22), 8.497 (7.65).

### Example 240

### N-[(trans)-1-oxo-2-phenyl-2-azaspiro[4.5]dec-8-yl]imidazo[1,2-b]pyridazine-3-carboxamide

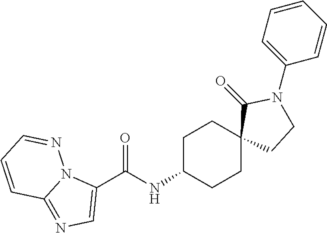 [SMILES: O=C(N[C@H]1CC[C@@]2(CCN(c3ccccc3)C2=O)CC1)c1cnc2cccnn12]

Was prepared in analogy to the synthesis of N-[(trans)-1-oxo-2-phenyl-2-azaspiro[4.5]dec-8-yl]pyrazolo[1,5-a]pyrimidine-3-carboxamide imidazo[1,2-b]pyridazine-3-carboxylic acid (58.4 mg, 358 μmol, Cas No 1308384-58-8) as coupling partner. For work-up, the reaction mixture was concentrated and the residue was stirred with methanol. The precipitate formed was collected by filtration and purified by preparative HPLC to give the title compound (29.0 mg).

LC-MS (Method 2): Rₜ=0.99 min; MS (ESIneg): m/z=388 [M−H]⁻

¹H-NMR (400 MHz, DMSO-d6) δ[ppm]: 1.484 (0.84), 1.495 (1.02), 1.515 (2.91), 1.526 (2.95), 1.546 (3.24), 1.556 (3.36), 1.577 (1.60), 1.586 (1.56), 1.633 (1.76), 1.667 (10.70), 1.700 (3.60), 1.707 (3.94), 1.733 (0.95), 1.740 (0.93), 1.964 (3.56), 1.971 (3.74), 1.995 (3.42), 2.002 (3.11), 2.088 (5.21), 2.105 (9.65), 2.123 (5.47), 2.327 (1.08), 2.669 (1.08), 3.791 (5.56), 3.808 (9.91), 3.825 (5.38), 3.854 (0.74), 3.873 (1.38), 3.884 (1.73), 3.892 (1.44), 3.902 (1.69), 3.912 (1.33), 3.930 (0.63), 7.116 (2.41), 7.134 (5.47), 7.153 (3.25), 7.358 (5.99), 7.380 (9.19), 7.398 (5.70), 7.451 (4.55), 7.462 (4.44), 7.474 (4.67), 7.485 (4.87), 7.687 (9.85), 7.707 (8.68), 8.306 (16.00), 8.338 (5.30), 8.342 (5.52), 8.361 (4.98), 8.365 (5.03), 8.479 (4.21), 8.498 (4.12), 8.786 (4.85), 8.789 (5.07), 8.797 (4.98), 8.800 (4.69).

### Example 241

### N-[(trans)-2-(2-chloro-4,6-difluorophenyl)-1-oxo-2-azaspiro[4.5]dec-8-yl]-7-(methoxymethyl)-5-methylpyrazolo[1,5-a]pyrimidine-3-carboxamide

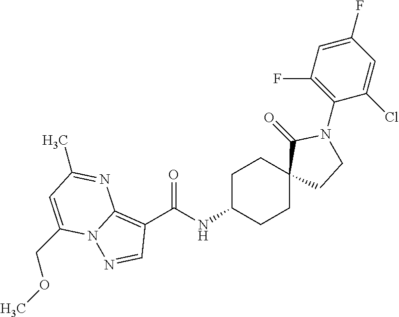 [SMILES: COCc1cc(C)nc2c(C(=O)N[C@H]3CC[C@@]4(CCN(c5c(F)cc(F)cc5Cl)C4=O)CC3)cnn12]

Benzotriazol-1-yl-oxytripyrrolidinophosphonium hexafluorophosphate (218 mg, 419 μmol) and N,N-diisopropylethylamine (300 μl, 1.7 mmol) were added to a mixture of 8-amino-2-(2-chloro-4,6-difluorophenyl)-2-azaspiro[4.5]decan-1-one (isomer 1) (200 mg, 55% purity, 349 μmol) and a mixture of regioisomers of 5-(methoxymethyl)-7-methylpyrazolo[1,5-a]pyrimidine-3-carboxylic acid (isomer 1) and 7-(methoxymethyl)-5-methylpyrazolo[1,5-a]pyrimidine-3-carboxylic acid (isomer 2) (92.8 mg, 419 μmol) in N,N-dimethylformamide (3.9 ml) and the mixture was stirred at room temperature for 12 h. For work-up, the mixture was concentrated and the residue was purified by preparative HPLC [Instrument: Waters Autopurificationsystem; column: Waters XBrigde C18 5μ 100×30 mm; eluent A: water+0.2 Vol-% aqueous ammonia (32%), eluent B: acetonitrile; gradient: 0.00-0.50 min 38% B (25->70 ml/min), 0.51-5.50 min 38-60% B (70 ml/min), DAD scan: 210-400 nm] to give N-[(trans)-2-(2-chloro-4,6-difluorophenyl)-1-oxo-2-azaspiro[4.5]dec-8-yl]-7-(methoxymethyl)-5-methylpyrazolo[1,5-a]pyrimidine-3-carboxamide (19.9 mg, 11% yield, Rₜ=4.54-4.79 min) and N-[(trans)-2-(2-chloro-4,6-difluorophenyl)-1-oxo-2-azaspiro[4.5]dec-8-yl]-5-(methoxymethyl)-7-methylpyrazolo[1,5-a]pyrimidine-3-carboxamide (61.7 mg, 34% yield, Rₜ=3.78-4.03 min)

### N-[(trans)-2-(2-chloro-4,6-difluorophenyl)-1-oxo-2-azaspiro[4.5]dec-8-yl]-7-(methoxymethyl)-5-methylpyrazolo[1,5-a]pyrimidine-3-carboxamide

LC-MS (Method 2): Rₜ=1.24 min; MS (ESIpos): m/z=518 [M+H]⁺

¹H-NMR (400 MHz, DMSO-d6) δ[ppm]: 1.469 (0.51), 1.481 (0.55), 1.499 (0.61), 1.510 (0.58), 1.671 (1.16), 1.681 (1.29), 1.694 (1.25), 1.710 (0.75), 1.722 (0.45), 1.994 (0.66), 2.003 (0.68), 2.013 (0.46), 2.025 (0.65), 2.035 (0.59), 2.059 (0.55), 2.197 (0.88), 2.214 (1.81), 2.232 (1.04), 2.327 (0.43), 2.518 (1.24), 2.523 (0.88), 2.669 (0.47), 2.693 (11.36), 3.521 (16.00), 3.540 (0.41), 3.547 (0.75), 3.633 (0.68), 3.640 (0.43), 3.651 (0.50), 3.657 (0.56), 4.961 (3.83), 4.964 (3.79), 7.165 (2.56), 7.504 (0.44), 7.511 (0.63), 7.527 (0.60), 7.535 (1.09), 7.542 (0.87), 7.546 (0.77), 7.549 (0.58), 7.554 (0.66), 7.558 (0.88), 7.564 (0.87), 7.568 (0.73), 7.571 (0.43), 7.575 (0.49), 7.983 (0.96), 8.002 (0.92), 8.504 (4.99), 8.514 (0.41).

### Example 242

### N-[(trans)-2-(2-chloro-4,6-difluorophenyl)-1-oxo-2-azaspiro[4.5]dec-8-yl]-5-(methoxymethyl)-7-methylpyrazolo[1,5-a]pyrimidine-3-carboxamide

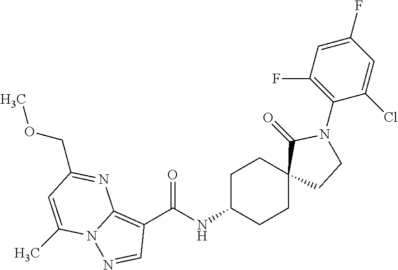 [SMILES: COCc1cc(C)n2ncc(C(=O)N[C@H]3CC[C@@]4(CCN(c5c(F)cc(F)cc5Cl)C4=O)CC3)c2n1]

Isolated as side product in the synthesis of N-[(trans)-2-(2-chloro-4,6-difluorophenyl)-1-oxo-2-azaspiro[4.5]dec-8-yl]-7-(methoxymethyl)-5-methylpyrazolo[1,5-a]pyrimidine-3-carboxamide: (61.7 mg, 34% yield, Rₜ=3.78-4.03 min)

LC-MS (Method 2): Rₜ=1.18 min; MS (ESIpos): m/z=518 [M+H]⁺

¹H-NMR (400 MHz, DMSO-d6) δ[ppm]: 1.453 (0.47), 1.464 (0.51), 1.483 (0.56), 1.493 (0.53), 1.672 (1.08), 1.682 (1.21), 1.694 (1.17), 1.710 (0.74), 1.722 (0.44), 1.994 (0.61), 2.003 (0.64), 2.026 (0.60), 2.035 (0.55), 2.190 (0.84), 2.207 (1.74), 2.224 (0.99), 2.518 (1.05), 2.523 (0.69), 2.802 (5.97), 2.804 (6.34), 3.463 (16.00), 3.527 (0.58), 3.532 (0.47), 3.551 (0.69), 3.636 (0.64), 3.654 (0.45), 3.660 (0.54), 4.665 (6.91), 7.242 (2.03), 7.244 (2.05), 7.512 (0.57), 7.528 (0.53), 7.536 (0.99), 7.543 (0.80), 7.547 (0.71), 7.550 (0.54), 7.554 (0.63), 7.558 (0.80), 7.565 (0.81), 7.568 (0.68), 7.575 (0.48), 7.960 (0.89), 7.979 (0.85), 8.560 (4.95).

### Example 243

### N-[(trans)-2-(2-chloro-4,6-difluorophenyl)-1-oxo-2-azaspiro[4.5]dec-8-yl]imidazo[1,2-b]pyridazine-3-carboxamide

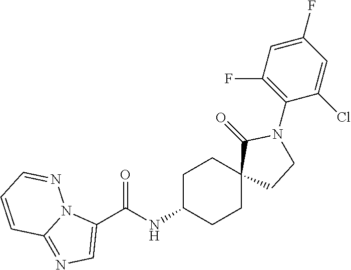 [SMILES: O=C(N[C@H]1CC[C@@]2(CCN(c3c(F)cc(F)cc3Cl)C2=O)CC1)c1cnc2cccnn12]

Benzotriazol-1-yl-oxytripyrrolidinophosphonium hexafluorophosphate (164 mg, 315 μmol) and N,N-diisopropylethylamine (230 μl, 1.3 mmol) were added to a mixture of 8-amino-2-(2-chloro-4,6-difluorophenyl)-2-azaspiro[4.5]decan-1-one (isomer 1) (150 mg, 55% purity 262 μmol) and imidazo[1,2-b]pyridazine-3-carboxylic acid (53.4 mg, 328 μmol, Cas No 1308384-58-8) in N,N-dimethylformamide (2.9 ml) and the mixture was stirred at room temperature overnight. For work-up, the mixture was concentrated and the residue was stirred with methanol. The precipitate formed was collected by filtration and purified by preparative HPLC to give the title compound (22.5 mg).

LC-MS (Method 2): Rₜ=1.04 min; MS (ESIpos): m/z=460 [M+H]⁺

¹H-NMR (400 MHz, DMSO-d6) δ[ppm]: 1.502 (0.46), 1.515 (0.48), 1.531 (1.52), 1.544 (1.59), 1.561 (1.93), 1.573 (1.84), 1.591 (1.08), 1.603 (0.95), 1.677 (3.95), 1.686 (4.32), 1.694 (4.10), 1.710 (2.69), 1.721 (1.59), 1.734 (0.56), 1.744 (0.48), 1.976 (2.04), 1.984 (2.17), 2.007 (2.04), 2.014 (1.87), 2.193 (2.60), 2.211 (5.51), 2.230 (3.28), 2.322 (0.76), 2.326 (1.06), 2.332 (0.78), 2.518 (8.68), 2.522 (6.51), 2.539 (1.74), 2.664 (0.76), 2.669 (1.09), 2.673 (0.80), 3.509 (0.80), 3.527 (1.93), 3.532 (1.67), 3.544 (1.26), 3.551 (2.35), 3.567 (1.17), 3.618 (1.19), 3.634 (2.11), 3.637 (1.78), 3.642 (1.39), 3.653 (1.61), 3.658 (1.87), 3.677 (0.85), 3.861 (0.43), 3.871 (0.48), 3.879 (0.85), 3.889 (1.06), 3.898 (0.87), 3.908 (1.04), 3.917 (0.82), 3.937 (0.41), 7.450 (5.01), 7.461 (4.56), 7.473 (4.78), 7.485 (5.14), 7.504 (1.41), 7.511 (2.00), 7.527 (1.84), 7.535 (3.45), 7.542 (2.69), 7.546 (2.41), 7.549 (1.89), 7.553 (2.21), 7.558 (2.87), 7.564 (2.80), 7.568 (2.34), 7.570 (1.39), 7.575 (1.80), 8.303 (16.00), 8.338 (4.97), 8.342 (5.49), 8.361 (5.01), 8.364 (4.62), 8.480 (2.93), 8.499 (2.89), 8.787 (3.80), 8.791 (3.95), 8.798 (3.93), 8.802 (3.65).

### Example 244

### N-[(trans)-2-(2-chloro-4,6-difluorophenyl)-1-oxo-2-azaspiro[4.5]dec-8-yl]pyrazolo[1,5-a]pyrimidine-3-carboxamide

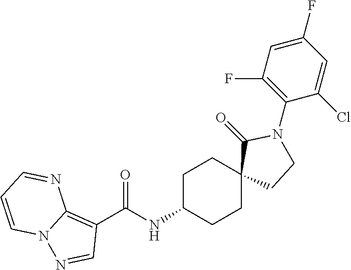 [SMILES: O=C(N[C@H]1CC[C@@]2(CCN(c3c(F)cc(F)cc3Cl)C2=O)CC1)c1cnn2cccnc12]

Was prepared in analogy to the synthesis of N-[(trans)-2-(2-chloro-4,6-difluorophenyl)-1-oxo-2-azaspiro[4.5]dec-8-yl]imidazo[1,2-b]pyridazine-3-carboxamide using pyrazolo[1,5-a]pyrimidine-3-carboxylic acid (71.3 mg, 437 μmol, Cas No 25940-35-6) as coupling partner to give the title compound (47.0 mg).

LC-MS (Method 2): Rₜ=1.07 min; MS (ESIpos): m/z=460 [M+H]⁺

¹H-NMR (400 MHz, DMSO-d₆): δ [ppm]=9.33 (dd, 1H), 8.84 (dd, 1H), 8.59 (s, 1H), 7.84 (d, 1H), 7.58-7.51 (m, 2H), 7.28 (dd, 1H), 3.91-3.81 (m, 1H), 3.69-3.61 (m, 1H), 3.58-3.50 (m, 1H), 2.22 (t, 2H), 2.03-1.95 (m, 2H), 1.75-1.63 (m, 4H), 1.57-1.44 (m, 2H)

### Example 245

### N-[(trans)-2-(3,5-difluorophenyl)-1-oxo-2-azaspiro[4.5]dec-8-yl]pyrazolo[1,5-a]pyrimidine-3-carboxamide

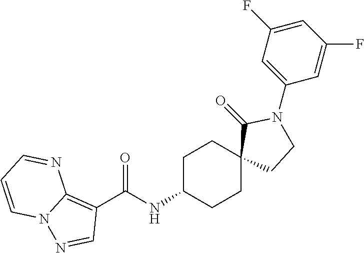 [SMILES: O=C(N[C@H]1CC[C@@]2(CCN(c3cc(F)cc(F)c3)C2=O)CC1)c1cnn2cccnc12]

Benzotriazol-1-yl-oxytripyrrolidinophosphonium hexafluorophosphate (100 mg, 193 μmol) and N,N-diisopropylethylamine (140 μl, 800 μmol) were added to a mixture of 8-amino-2-(3,5-difluorophenyl)-2-azaspiro[4.5]decan-1-one (isomer 1) (50.0 mg, 161 μmol) and pyrazolo[1,5-a]pyrimidine-3-carboxylic acid (28.8 mg, 177 μmol, Cas No 25940-35-6) in N,N-dimethylformamide (1.8 ml) and the mixture was stirred at room temperature for 12 h. For work-up, the mixture was concentrated and the residue was purified by preparative HPLC (Method 9) to give the title compound (44.7 mg).

LC-MS (Method 2): Rₜ=1.11 min; MS (ESIpos): m/z=426 [M+H]⁺

¹H-NMR (400 MHz, DMSO-d6) δ[ppm]: 1.443 (0.92), 1.460 (2.48), 1.472 (2.67), 1.490 (3.02), 1.502 (2.97), 1.520 (1.46), 1.532 (1.31), 1.650 (8.75), 1.658 (11.43), 1.680 (3.50), 1.688 (3.70), 1.713 (0.83), 1.947 (3.50), 1.969 (3.21), 2.092 (4.81), 2.109 (8.85), 2.127 (5.06), 2.323 (2.19), 2.327 (2.82), 2.331 (2.09), 2.523 (8.56), 2.665 (2.24), 2.669 (2.92), 2.673 (2.19), 3.159 (4.23), 3.172 (4.33), 3.788 (5.11), 3.806 (9.48), 3.823 (5.84), 3.835 (1.85), 3.845 (1.41), 3.854 (1.60), 3.865 (1.26), 4.098 (0.97), 4.110 (1.02), 6.982 (0.97), 6.989 (1.85), 6.994 (1.22), 7.006 (2.04), 7.011 (3.70), 7.017 (2.24), 7.034 (1.80), 7.041 (1.07), 7.260 (4.18), 7.271 (4.33), 7.277 (4.33), 7.288 (4.33), 7.500 (4.91), 7.506 (5.98), 7.526 (5.79), 7.531 (5.06), 7.543 (1.02), 7.810 (4.18), 7.830 (4.09), 8.580 (16.00), 8.816 (4.91), 8.821 (5.25), 8.827 (5.06), 8.831 (4.77), 9.309 (5.01), 9.314 (4.72), 9.327 (4.91), 9.331 (4.81).

### Example 246

### N-[(trans)-2-(4-chloropyrid in-3-yl)-1-oxo-2-azaspiro[4.5]dec-8-yl]pyrazolo[1,5-a]pyrimidine-3-carboxamide

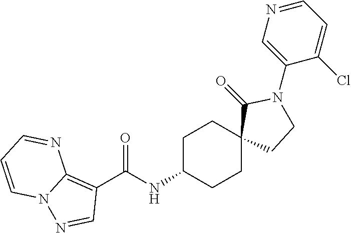 [SMILES: O=C(N[C@H]1CC[C@@]2(CCN(c3cnccc3Cl)C2=O)CC1)c1cnn2cccnc12]

Benzotriazol-1-yl-oxytripyrrolidinophosphonium hexafluorophosphate (105 mg, 201 μmol) and N,N-diisopropylethylamine (150 μl, 840 μmol) were added to a mixture of 8-amino-2-(4-chloropyridin-3-yl)-2-azaspiro[4.5]decan-1-one (isomer 1) (67.0 mg, 168 μmol) and pyrazolo[1,5-a]pyrimidine-3-carboxylic acid (30.1 mg, 184 μmol, Cas No 25940-35-6) in N,N-dimethylformamide (710 μl) and the mixture was stirred at room temperature overnight. For work-up, water was added and the precipitate formed was collected by filtration, washed with methanol and dried to give the title compound (24.0 mg).

LC-MS (Method 2): Rₜ=0.84 min; MS (ESIpos): m/z=425 [M+H]⁺

¹H-NMR (400 MHz, DMSO-d₆): δ [ppm]=9.33 (dd, 1H), 8.84 (dd, 1H), 8.62 (s, 1H), 8.59 (s, 1H), 8.52 (d, 1H), 7.84 (d, 1H), 7.71 (d, 1H), 7.28 (dd, 1H), 3.92-3.80 (m, 1H), 3.72 (t, 2H), 2.21 (t, 2H), 2.04-1.95 (m, 2H), 1.78-1.65 (m, 4H), 1.59-1.44 (m, 2H)

### Example 247

### N-[(trans)-2-(2-chloro-4-fluorophenyl)-1-oxo-2-azaspiro[4.5]dec-8-yl]-1-methyl-1H-indazole-3-carboxamide

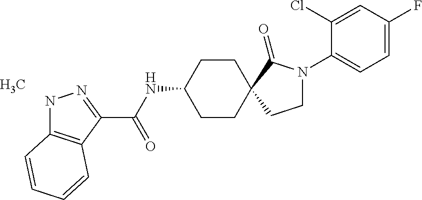

To a solution of 8-amino-2-(2-chloro-4-fluorophenyl)-2-azaspiro[4.5]decan-1-one (isomer 1) (44.5 mg, 150 μmol) in N-methylpyrrolidone (1.0 ml) was added 1-methyl-1H-indazole-3-carboxylic acid (34.4 mg, 195 μmol) in N-methylpyrrolidone (1.0 ml). HATU (74.1 mg, 195 μmol) in N-methylpyrrolidone (0.5 ml) and N,N-diisopropylethylamine (50.4 mg, 390 μmol) in N-methylpyrrolidone (0.5 ml) added. The reaction was shaked at room temperature for 16 h. The reaction mixture was subjected to preparative HPLC purification without further workup to give 14.7 mg (22%) of the title compound.

Instrument MS: Waters ZQ; Instrument HPLC: Waters UPLC Acquity; Column: Acquity BEH C₁₈ (Waters), 50 mm×2.1 mm, 1.7 μm; eluent A: water+0.1 vol % formic acid, eluent B: acetonitrile (Lichrosolv Merck); gradient: 0.0 min 99% A—1.6 min 1% A—1.8 min 1% A—1.81 min 99% A—2.0 min 99% A; temperature: 60° C.; flow: 0.8 mL/min; UV-Detection PDA 210-400 nm.

LC-MS: Rₜ=1.24 min; MS (ESIpos): m/z=455 [M+H]⁺.

The following examples were prepared in analogy to example 247:

| | | MS | |
|---|---|---|---|
| | | (ESIpos) | Retention |
| Example | Structure | m/z | Time |
| No | Name | [M + H]+ | [min] |
| 248 | 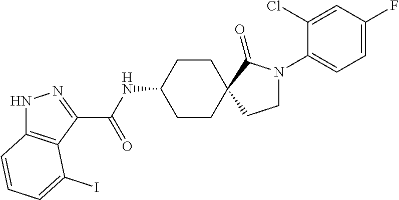 [SMILES: O=C(N[C@H]1CC[C@@]2(CCN(c3ccc(F)cc3Cl)C2=O)CC1)c1n[nH]c2cccc(I)c12] N-[(trans)-2-(2-chloro-4-fluorophenyl)-1-oxo-2- azaspiro[4.5]dec-8-yl]-4-iodo-1H-indazole-3- carboxamide | 567 | 1.16 |
| 249 | 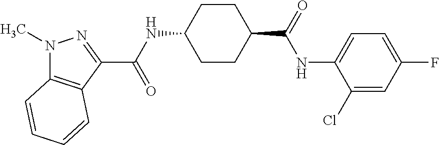 [SMILES: Cn1nc(C(=O)N[C@H]2CC[C@H](C(=O)Nc3ccc(F)cc3Cl)CC2)c2ccccc21] N-{trans-4-[(2-chloro-4- fluorophenyl)carbamoyl]cyclohexyl}-1-methyl-1H- indazole-3-carboxamide | 429 | 1.21 |
| 250 | 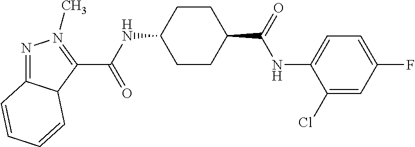 N-{trans-4-[(2-chloro-4- fluorophenyl)carbamoyl]cyclohexyl}-2-methyl-2H- indazole-3-carboxamide | 429 | 1.21 |
| 251 | 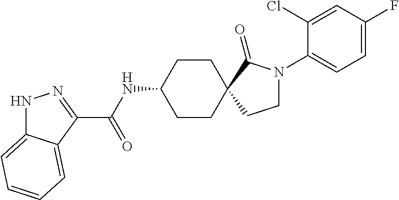 [SMILES: O=C(N[C@H]1CC[C@@]2(CCN(c3ccc(F)cc3Cl)C2=O)CC1)c1n[nH]c2ccccc12] N-[trans-2-(2-chloro-4-fluorophenyl)-1-oxo-2- azaspiro[4.5]dec-8-yl]-1H-indazole-3-carboxamide | 441 | 1.13 |
| 252 | 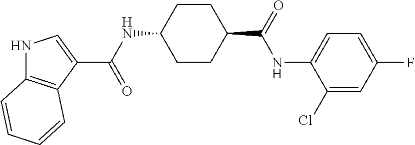 [SMILES: O=C(N[C@H]1CC[C@H](C(=O)Nc2ccc(F)cc2Cl)CC1)c1c[nH]c2ccccc12] N-{trans-4-[(2-chloro-4- fluorophenyl)carbamoyl]cyclohexyl}-1H-indole-3- carboxamide | 414 | 1.10 |
| 253 | 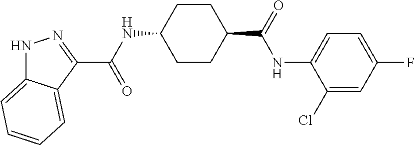 [SMILES: O=C(N[C@H]1CC[C@H](C(=O)Nc2ccc(F)cc2Cl)CC1)c1n[nH]c2ccccc12] N-{trans-4-[(2-chloro-4- fluorophenyl)carbamoyl]cyclohexyl}-1H-indazole-3- carboxamide | 415 | 1.12 |
| 254 | 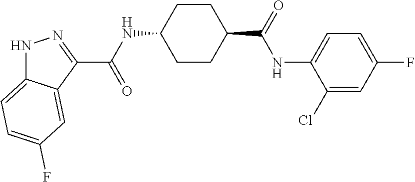 [SMILES: O=C(N[C@H]1CC[C@H](C(=O)Nc2ccc(F)cc2Cl)CC1)c1n[nH]c2ccc(F)cc12] N-{trans-4-[(2-chloro-4- fluorophenyl)carbamoyl]cyclohexyl}-5-fluoro-1H- indazole-3-carboxamide | 433 | 1.15 |
| 255 | 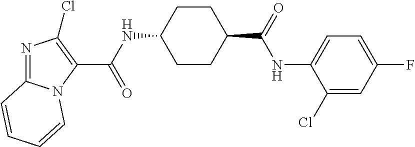 [SMILES: O=C(N[C@H]1CC[C@H](C(=O)Nc2ccc(F)cc2Cl)CC1)c1c(Cl)nc2ccccn12] 2-chloro-N-{trans-4-[(2-chloro-4- fluorophenyl)carbamoyl]cyclohexyl}imidazo[1,2- a]pyridine-3-carboxamide | 449 | 1.19 |

### Example 256

### N-[(trans)-2-(2-chlorophenyl)-1-oxo-2-azaspiro[4.5]dec-8-yl]pyrazolo[1,5-a]pyrimidine-3-carboxamide

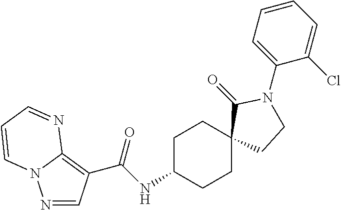 [SMILES: O=C(N[C@H]1CC[C@@]2(CCN(c3ccccc3Cl)C2=O)CC1)c1cnn2cccnc12]

Benzotriazol-1-yl-oxytripyrrolidinophosphonium hexafluorophosphate (187 mg, 359 μmol) and N,N-diisopropylethylamine (230 μl, 1.3 mmol) were added to a mixture of pyrazolo[1,5-a]pyrimidine-3-carboxylic acid (54.8 mg, 326 μmol Cas No Cas No 25940-35-6) and 8-amino-2-(2-chlorophenyl)-2-azaspiro[4.5]decan-1-one (isomer 1) (100 mg, 359 μmol) in N,N-dimethylformamide (3.3 ml) and the mixture was stirred at room temperature for 20 h. For work-up, water (50 ml) and sodium chloride were added and the resulting precipitate was collected by filtration, washed with water and dried to give the title compound (123 mg).

LC-MS (Method 2): Rₜ=1.02 min; MS (ESIpos): m/z=424.2 [M+H]⁺

¹H-NMR (400 MHz, DMSO-d6) δ[ppm]: 1.471 (1.43), 1.496 (2.10), 1.514 (1.91), 1.678 (7.16), 1.686 (8.14), 1.703 (5.20), 1.963 (2.53), 1.971 (2.64), 1.994 (2.46), 2.003 (2.30), 2.156 (4.42), 2.173 (8.35), 2.190 (4.72), 2.523 (1.96), 3.630 (4.70), 3.648 (8.33), 3.665 (4.63), 3.837 (1.25), 3.856 (1.20), 7.262 (4.32), 7.272 (4.20), 7.279 (4.28), 7.290 (4.37), 7.362 (1.10), 7.373 (2.04), 7.375 (1.87), 7.380 (1.03), 7.386 (3.44), 7.390 (1.65), 7.394 (1.97), 7.400 (1.75), 7.404 (7.11), 7.408 (10.86), 7.412 (12.76), 7.419 (3.82), 7.422 (4.75), 7.426 (1.75), 7.559 (3.02), 7.562 (4.95), 7.566 (2.85), 7.577 (2.43), 7.581 (3.87), 7.584 (2.37), 7.818 (3.48), 7.838 (3.41), 8.581 (16.00), 8.823 (4.75), 8.827 (5.36), 8.833 (4.95), 8.838 (4.83), 9.311 (4.94), 9.315 (5.14), 9.328 (4.98), 9.333 (5.03).

### Example 257

### N-[(trans)-2-(2-chlorophenyl)-1-oxo-2-azaspiro[4.5]dec-8-yl]-5,7-dimethylpyrazolo[1,5-a]pyrimidine-3-carboxamide

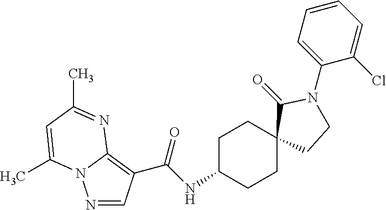 [SMILES: Cc1cc(C)n2ncc(C(=O)N[C@H]3CC[C@@]4(CCN(c5ccccc5Cl)C4=O)CC3)c2n1]

Benzotriazol-1-yl-oxytripyrrolidinophosphonium hexafluorophosphate (187 mg, 359 μmol) and N,N-diisopropylethylamine (230 μl, 1.3 mmol) were added to a mixture of 5,7-dimethylpyrazolo[1,5-a]pyrimidine-3-carboxylic acid (65.6 mg, 326 μmol, Cas No 90349-23-8) and 8-amino-2-(2-chlorophenyl)-2-azaspiro[4.5]decan-1-one (isomer 1) (100 mg, 359 μmol) in N,N-dimethylformamide (3.3 ml) and the mixture was stirred at room temperature for 20 h.

For work-up, water (35 ml) was added and the precipitate formed was collected by filtration, washed with water and dried to give the title compound (125 mg).

LC-MS (Method 2): Rₜ=1.17 min; MS (ESIpos): m/z=452.2 [M+H]⁺

¹H-NMR (400 MHz, DMSO-d6) δ[ppm]: 1.765 (2.78), 1.774 (2.11), 1.805 (1.20), 2.215 (1.71), 2.232 (3.19), 2.249 (1.84), 2.572 (1.37), 2.679 (16.00), 2.786 (9.37), 2.788 (9.83), 3.688 (1.89), 3.705 (3.26), 3.723 (1.86), 7.093 (3.17), 7.095 (3.17), 7.381 (0.65), 7.388 (0.78), 7.396 (1.46), 7.401 (0.91), 7.405 (1.50), 7.413 (5.09), 7.415 (5.99), 7.420 (3.48), 7.429 (1.24), 7.430 (1.22), 7.552 (1.26), 7.555 (1.57), 7.559 (0.88), 7.569 (0.86), 7.572 (1.60), 7.576 (0.90), 8.046 (1.28), 8.065 (1.25), 8.265 (6.38), 8.496 (7.10).

### Example 258

### N⁵-[(trans)-2-(2-chlorophenyl)-1-oxo-2-azaspiro[4.5]dec-8-yl]-N⁴-ethyl-1H-imidazole-4,5-dicarboxamide

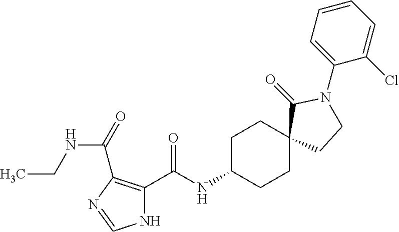 [SMILES: CCNC(=O)c1nc[nH]c1C(=O)N[C@H]1CC[C@@]2(CCN(c3ccccc3Cl)C2=O)CC1]

A mixture of phenyl 5-{[2-(2-chlorophenyl)-1-oxo-2-azaspiro[4.5]dec-8-yl]carbamoyl}-1H-imidazole-4-carboxylate (isomer 1) (150 mg, 259 μmol) and ethanamine (650 μl, 2.0 M, 1.3 mmol, Cas No 75-04-7) in tetrahydrofuran (4.2 ml) was stirred for 20 h at room temperature. For work-up, brine was added and the mixture was extracted with ethyl acetate. The combined organic phases were filtrated through a silicone filter, concentrated and the residue was purified by flash chromatography (25 g Snap-cartridge, dichloromethane/ethanol-gradient, 0%->18% ethanol) to give the title compound (43.0 mg).

LC-MS (Method 2): Rₜ=1.03 min; MS (ESIpos): m/z=444.1 [M+H]⁺

¹H-NMR (500 MHz, DMSO-d6) δ[ppm]: 1.115 (5.09), 1.129 (9.50), 1.143 (5.04), 1.669 (8.55), 1.987 (1.27), 2.131 (4.09), 2.145 (7.70), 2.159 (4.24), 2.361 (1.53), 2.518 (3.63), 2.522 (2.61), 2.634 (1.46), 3.284 (1.44), 3.298 (4.72), 3.310 (6.55), 3.628 (6.09), 3.641 (10.96), 3.655 (5.87), 7.364 (1.46), 7.371 (1.97), 7.376 (2.17), 7.383 (3.95), 7.387 (1.90), 7.391 (2.63), 7.398 (7.38), 7.405 (16.00), 7.411 (4.77), 7.416 (3.73), 7.419 (2.19), 7.557 (4.72), 7.560 (6.33), 7.573 (4.12), 7.574 (4.97), 7.810 (14.59), 13.206 (2.46).

### Example 259

### N⁵-[(trans)-2-(2-chlorophenyl)-1-oxo-2-azaspiro[4.5]dec-8-yl]-N⁴-(2-methoxyethyl)-1H-imidazole-4,5-dicarboxamide

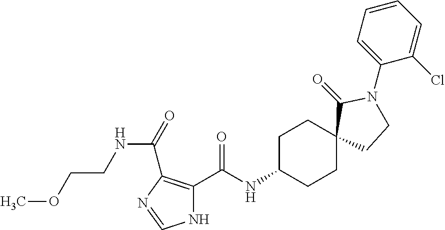 [SMILES: COCCNC(=O)c1nc[nH]c1C(=O)N[C@H]1CC[C@@]2(CCN(c3ccccc3Cl)C2=O)CC1]

A mixture of phenyl 5-{[2-(2-chlorophenyl)-1-oxo-2-azaspiro[4.5]dec-8-yl]carbamoyl}-1H-imidazole-4-carboxylate (isomer 1) (150 mg, 259 μmol) and 2-methoxyethanamine (97.1 mg, 1.29 mmol, Cas No 109-85-3) in tetrahydrofuran (4.2 ml) was stirred for 20 h at room temperature. For work-up, brine was added and the resulting precipitate was collected by filtration and washed with water. The product was purified by flash chromatography (2×) (11 g NH2-cartridge, dichloromethane/ethanol-gradient, 0%->14% ethanol, followed by 12 g Snap Cartridge, dichloromethane/ethanol-gradient, 0%->25% ethanol) to give the title compound (69 mg).

LC-MS (Method 2): Rₜ=0.98 min; MS (ESIpos): m/z=474.2 [M+H]⁺

¹H-NMR (500 MHz, DMSO-d6) δ[ppm]: 1.157 (0.47), 1.171 (0.91), 1.186 (0.50), 1.669 (1.30), 1.987 (1.96), 2.130 (0.57), 2.143 (1.03), 2.157 (0.57), 3.274 (16.00), 3.332 (9.69), 3.626 (1.13), 3.640 (1.98), 3.654 (1.12), 7.375 (0.46), 7.382 (0.91), 7.386 (0.40), 7.390 (0.50), 7.397 (1.73), 7.404 (2.65), 7.407 (2.10), 7.410 (0.80), 7.416 (0.81), 7.419 (0.50), 7.557 (0.80), 7.559 (1.32), 7.561 (0.72), 7.572 (0.75), 7.574 (0.91), 7.576 (0.66), 7.823 (2.53), 13.241 (0.65).

### Example 260

### N⁵-[(trans)-2-(2-chlorophenyl)-1-oxo-2-azaspiro[4.5]dec-8-yl]-N⁴-[2-(4-methylpiperidin-1-yl)ethyl]-1H-imidazole-4,5-dicarboxamide

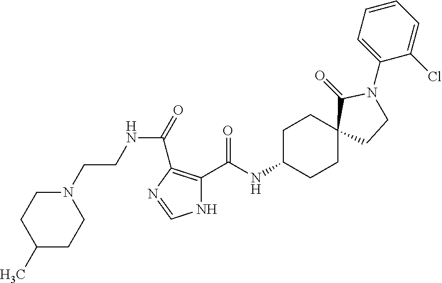 [SMILES: CC1CCN(CCNC(=O)c2nc[nH]c2C(=O)N[C@H]2CC[C@@]3(CCN(c4ccccc4Cl)C3=O)CC2)CC1]

A mixture of phenyl 5-{[2-(2-chlorophenyl)-1-oxo-2-azaspiro[4.5]dec-8-yl]carbamoyl}-1H-imidazole-4-carboxylate (isomer 1) (150 mg, 259 μmol) and 2-(4-methylpiperidin-1-yl)ethanamine (184 mg, 1.29 mmol, Cas No 14156-95-7) in tetrahydrofuran (4.2 ml) was stirred for 20 h at room temperature. For work-up, brine was added and the mixture was extracted with ethyl acetate. The combined organic phases were filtrated through a silicone filter, concentrated and the residue was purified by flash chromatography (2×) (11 g NH2-cartridge, dichloromethane/ethanol-gradient, 0%->18% ethanol, followed 25 g Snap-cartridge, dichloromethane/ethanol-gradient, 0%->30% ethanol) to give the title compound (56 mg).

LC-MS (Method 2): Rₜ=1.26 min; MS (ESIpos): m/z=541.4 [M+H]⁺

¹H-NMR (500 MHz, DMSO-d6) δ[ppm]: 0.874 (14.56), 0.888 (15.19), 1.133 (1.58), 1.309 (1.15), 1.553 (2.88), 1.577 (2.63), 1.669 (5.85), 1.897 (2.00), 1.920 (3.56), 1.941 (2.12), 2.129 (3.20), 2.142 (6.07), 2.156 (3.37), 2.357 (0.79), 2.361 (1.15), 2.365 (0.83), 2.441 (4.93), 2.455 (2.79), 2.514 (3.28), 2.518 (2.92), 2.522 (2.29), 2.630 (0.76), 2.634 (1.08), 2.638 (0.79), 2.841 (3.65), 2.864 (3.53), 3.375 (1.96), 3.387 (4.95), 3.399 (4.88), 3.412 (1.87), 3.626 (5.13), 3.641 (9.05), 3.654 (5.11), 7.364 (1.78), 7.371 (1.94), 7.375 (2.11), 7.379 (1.69), 7.382 (4.07), 7.386 (1.91), 7.390 (2.45), 7.397 (8.13), 7.404 (12.11), 7.407 (10.28), 7.411 (3.91), 7.416 (3.98), 7.419 (2.48), 7.557 (4.00), 7.559 (6.26), 7.561 (3.64), 7.572 (3.28), 7.574 (4.39), 7.576 (3.35), 7.815 (16.00), 13.219 (2.47).

### Example 261

### N⁵-[(trans)-2-(2-chlorophenyl)-1-oxo-2-azaspiro[4.5]dec-8-yl]-1H-imidazole-4,5-dicarboxamide

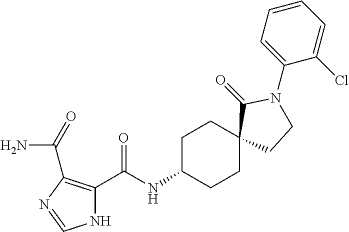 [SMILES: NC(=O)c1nc[nH]c1C(=O)N[C@H]1CC[C@@]2(CCN(c3ccccc3Cl)C2=O)CC1]

phenyl 5-{[2-(2-chlorophenyl)-1-oxo-2-azaspiro[4.5]dec-8-yl]carbamoyl}-1H-imidazole-4-carboxylate (isomer 1) (95.0 mg, 193 μmol) in a solution of ammonia in THF (4.8 ml, 0.40 M, 1.9 mmol) and tetrahydrofuran (5.0 ml) was stirred for 20 h at 50° C. in a microwave. Ammonia in THF (4.8 ml, 0.40 M, 19 mmol) was added and the mixture was heated to 100° C. for 90 min in a microwave reactor. For work-up, the reaction mixture was concentrated and the residue was purified by preparative HPLC (Method 8). The organic solvent of the combined product containing fractions was removed under reduced pressure and the resulting precipitate was collected by filtration and dried under high vacuum to give the title compound (41 mg).

LC-MS (Method 1): Rₜ=0.90 min; MS (ESIpos): m/z=416.1 [M+H]⁺

¹H-NMR (400 MHz, DMSO-d6) δ[ppm]: 1.391 (0.93), 1.663 (8.60), 1.787 (0.91), 1.986 (1.23), 2.115 (6.11), 2.123 (1.65), 2.132 (11.61), 2.149 (6.45), 2.322 (0.81), 2.327 (1.17), 2.332 (0.85), 2.518 (3.59), 2.523 (2.56), 2.665 (0.87), 2.669 (1.23), 2.673 (0.85), 3.622 (6.49), 3.630 (1.79), 3.639 (11.41), 3.649 (1.71), 3.656 (6.27), 3.760 (0.91), 7.359 (1.69), 7.368 (2.52), 7.372 (2.58), 7.376 (1.69), 7.381 (5.42), 7.385 (2.10), 7.390 (2.72), 7.392 (3.53), 7.396 (4.47), 7.400 (12.01), 7.402 (16.00), 7.405 (15.60), 7.411 (5.98), 7.416 (4.82), 7.421 (1.95), 7.547 (0.73), 7.554 (4.72), 7.558 (8.00), 7.561 (3.77), 7.572 (3.77), 7.576 (5.78), 7.579 (3.75), 7.771 (1.31), 7.803 (5.74), 7.976 (0.73), 13.194 (2.44).

### Example 262

### N-[(trans)-2-(2-chlorophenyl)-1-oxo-2-azaspiro[4.5]dec-8-yl]-4-[(3-fluoroazetidin-1-yl)carbonyl]-1H-imidazole-5-carboxamide

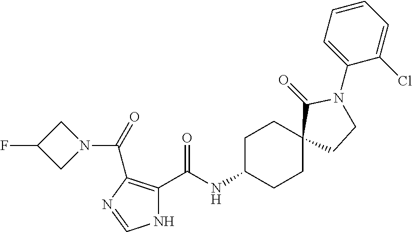 [SMILES: O=C(N[C@H]1CC[C@@]2(CCN(c3ccccc3Cl)C2=O)CC1)c1[nH]cnc1C(=O)N1CC(F)C1]

A mixture of phenyl 5-{[2-(2-chlorophenyl)-1-oxo-2-azaspiro[4.5]dec-8-yl]carbamoyl}-1H-imidazole-4-carboxylate (isomer 1) (95.0 mg, 193 μmol), 3-fluoroazetidine hydrochloride (43.0 mg, 385 μmol, Cas No 617718-46-4) and triethylamine (81 μl) in tetrahydrofuran (5 ml) was stirred for 20 h at 50° C. For work-up, the react ion mixture was concentrated and the residue was purified by preparative HPLC (Method 8). The organic solvent of the combined product containing fractions was removed under reduced pressure and the resulting precipitate was collected by filtration and dried under high vacuum to give the title compound (56 mg).

LC-MS (Method 2): Rₜ=1.05 min; MS (ESIpos): m/z=474.2 [M+H]⁺

¹H-NMR (400 MHz, DMSO-d6) δ[ppm]: 1.353 (1.51), 1.381 (2.22), 1.406 (1.77), 1.662 (6.55), 1.671 (7.81), 1.684 (6.56), 1.693 (6.35), 1.959 (2.61), 1.968 (2.40), 1.981 (2.26), 1.991 (2.40), 2.124 (4.58), 2.141 (8.59), 2.158 (4.83), 2.327 (0.78), 2.669 (0.82), 3.623 (5.00), 3.640 (9.05), 3.657 (4.75), 3.711 (1.12), 3.721 (1.39), 3.730 (1.12), 3.739 (1.36), 3.749 (1.05), 4.076 (1.03), 4.105 (1.28), 4.112 (0.93), 4.138 (1.11), 4.168 (1.24), 4.379 (0.83), 4.389 (0.94), 4.394 (0.91), 4.410 (0.79), 4.429 (1.01), 4.444 (0.93), 4.448 (0.89), 4.464 (0.78), 4.588 (0.94), 4.619 (1.19), 4.641 (0.78), 4.648 (1.01), 4.680 (1.12), 4.899 (0.94), 4.903 (0.90), 4.940 (0.89), 4.954 (0.87), 5.351 (0.86), 5.358 (1.16), 5.365 (1.36), 5.373 (1.10), 5.503 (1.16), 5.510 (1.36), 5.517 (1.12), 5.525 (0.79), 7.360 (1.06), 7.369 (1.69), 7.373 (1.69), 7.377 (1.07), 7.382 (3.41), 7.387 (1.44), 7.393 (2.42), 7.397 (3.31), 7.401 (8.19), 7.404 (12.89), 7.407 (12.68), 7.413 (4.25), 7.417 (3.57), 7.422 (1.39), 7.555 (3.04), 7.559 (5.04), 7.562 (2.94), 7.573 (2.26), 7.577 (4.01), 7.580 (2.37), 7.832 (16.00), 11.072 (3.63), 11.091 (3.47), 13.301 (3.64).

### Example 263

### N-[(trans)-2-(2-chlorophenyl)-1-oxo-2-azaspiro[4.5]dec-8-yl]-4-(2-oxa-6-azaspiro[3.3]hept-6-ylcarbonyl)-1H-imidazole-5-carboxamide

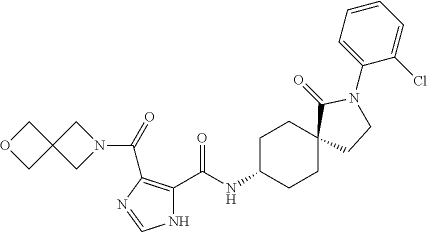 [SMILES: O=C(N[C@H]1CC[C@@]2(CCN(c3ccccc3Cl)C2=O)CC1)c1[nH]cnc1C(=O)N1CC2(COC2)C1]

A mixture of phenyl 5-{[2-(2-chlorophenyl)-1-oxo-2-azaspiro[4.5]dec-8-yl]carbamoyl}-1H-imidazole-4-carboxylate (isomer 1) (95.0 mg, 193 μmol), 2-oxa-6-azaspiro[3.3]heptane ethanedioate (2:1) (55.6 mg, 193 μmol, Cas No 1045709-32-7) and triethylamine (81 μl) in tetrahydrofuran (1.9 ml) was stirred for 20 h at 50° C. 2-oxa-6-azaspiro[3.3]heptane ethanedioate (27.8 mg, 96.5 μmol, Cas No 1045709-32-7) was added and the mixture was stirred for 3 d at 50° C. For work-up, the reaction mixture was concentrated and the residue was purified by preparative HPLC (Method 8). The product crystalized from water/acetonitrile and was collected by filtration and dried under high vacuum to give the title compound (40 mg).

LC-MS (Method 1): Rₜ=0.95 min; MS (ESIpos): m/z=498.3 [M+H]⁺

¹H-NMR (400 MHz, DMSO-d6) δ[ppm]: 1.369 (1.75), 1.388 (1.21), 1.398 (1.25), 1.656 (4.17), 1.666 (5.16), 1.679 (4.75), 1.687 (4.65), 1.939 (1.87), 1.948 (1.95), 1.959 (1.34), 1.971 (1.89), 1.981 (1.69), 2.123 (3.41), 2.140 (6.31), 2.157 (3.52), 2.322 (0.84), 2.327 (1.21), 2.332 (0.88), 2.518 (3.35), 2.523 (2.45), 2.665 (0.84), 2.669 (1.25), 2.673 (0.86), 3.622 (3.70), 3.640 (6.33), 3.657 (3.52), 4.251 (8.97), 4.674 (2.57), 4.692 (11.37), 4.700 (11.25), 4.717 (2.53), 4.768 (8.10), 7.359 (0.99), 7.369 (1.58), 7.373 (1.44), 7.377 (0.90), 7.383 (3.06), 7.386 (1.21), 7.391 (1.32), 7.393 (1.93), 7.397 (2.80), 7.401 (6.50), 7.404 (9.54), 7.407 (9.03), 7.413 (3.17), 7.418 (2.86), 7.422 (1.13), 7.556 (2.74), 7.559 (4.32), 7.562 (2.41), 7.573 (2.12), 7.577 (3.31), 7.580 (2.04), 7.811 (16.00), 11.167 (2.43), 11.185 (2.28), 13.242 (2.37).

### Example 264

### N⁵-[(trans)-2-(2-chlorophenyl)-1-oxo-2-azaspiro[4.5]dec-8-yl]-N⁴-methyl-1H-imidazole-4,5-dicarboxamide

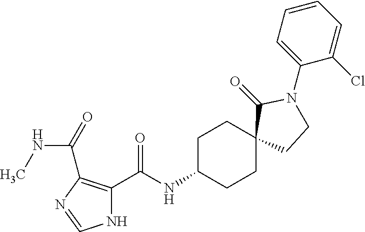 [SMILES: CNC(=O)c1nc[nH]c1C(=O)N[C@H]1CC[C@@]2(CCN(c3ccccc3Cl)C2=O)CC1]

A mixture of phenyl 5-{[2-(2-chlorophenyl)-1-oxo-2-azaspiro[4.5]dec-8-yl]carbamoyl}-1H-imidazole-4-carboxylate (isomer 1) (150 mg, 304 μmol) and methanamine in tetrahydrofuran (760 μl, 2.0 M, 1.5 mmol, Cas No 74-89-5) in tetrahydrofuran (4.9 ml) was stirred for 20 h at room temperature. For work-up, brine was added and the resulting precipitate was collected by filtration and washed with water. The crude product was purified by flash chromatography (dichloromethane/ethyl acetate gradient 0%->100% ethyl acetate) to give the title compound (16 mg).

LC-MS (Method 2): Rₜ=0.96 min; MS (ESIpos): m/z=430.4 [M+H]⁺

¹H-NMR (400 MHz, DMSO-d6) δ[ppm]: 1.154 (0.87), 1.171 (1.76), 1.189 (0.87), 1.669 (7.71), 1.987 (4.24), 2.129 (3.68), 2.146 (6.95), 2.163 (3.89), 2.322 (1.16), 2.326 (1.61), 2.332 (1.13), 2.518 (10.03), 2.522 (8.03), 2.664 (1.18), 2.669 (1.58), 2.673 (1.13), 2.810 (7.08), 2.820 (7.18), 3.625 (5.29), 3.642 (9.50), 3.660 (5.24), 4.017 (0.71), 4.034 (0.71), 7.359 (1.45), 7.369 (2.16), 7.373 (2.08), 7.377 (1.53), 7.383 (4.42), 7.386 (1.97), 7.393 (3.05), 7.397 (4.24), 7.401 (9.76), 7.404 (14.26), 7.407 (13.71), 7.413 (4.79), 7.418 (4.32), 7.422 (1.92), 7.556 (3.82), 7.559 (6.42), 7.563 (3.34), 7.573 (3.13), 7.577 (4.82), 7.580 (3.03), 7.811 (16.00), 13.213 (1.66).

### Example 265

### N-[(trans)-2-(2-chloro-4-fluoro-5-methyl phenyl)-1-oxo-2-azaspiro[4.5]dec-8-yl]imidazo[1,2-b]pyridazine-3-carboxamide

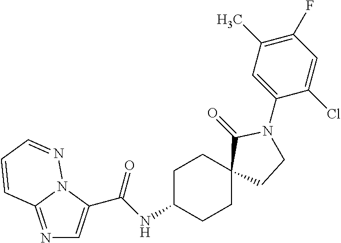 [SMILES: Cc1cc(N2CC[C@]3(CC[C@@H](NC(=O)c4cnc5cccnn45)CC3)C2=O)c(Cl)cc1F]

Benzotriazol-1-yl-oxytripyrrolidinophosphonium hexafluorophosphate (251 mg, 483 μmol) and N,N-diisopropylethylamine (310 μl, 1.8 mmol) were added to a mixture of 8-amino-2-(2-chloro-4-fluoro-5-methylphenyl)-2-azaspiro[4.5]decan-1-one (isomer 1) (150 mg, 483 μmol) and imidazo[1,2-b]pyridazine-3-carboxylic acid (73.8 mg, 439 μmol, Cas No, 1308384-58-8) in N,N-dimethylformamide (4.5 ml) and the mixture was stirred at room temperature for 20 h. For work-up, water (50 ml) was added and the precipitate formed was collected by filtration, washed with water and dried to give the title compound (188 mg).

LC-MS (Method 2): Rₜ=1.11 min; MS (ESIpos): m/z=456.2 [M+H]⁺

¹H-NMR (400 MHz, DMSO-d6) b [ppm]: 1.517 (1.42), 1.546 (2.07), 1.562 (2.08), 1.591 (1.07), 1.674 (7.31), 1.682 (8.14), 1.699 (4.65), 1.982 (2.69), 2.005 (2.53), 2.140 (3.93), 2.157 (7.57), 2.174 (4.17), 2.224 (15.84), 2.229 (16.00), 3.597 (4.25), 3.615 (7.46), 3.632 (3.98), 3.880 (1.20), 3.899 (1.17), 7.373 (4.11), 7.393 (4.03), 7.450 (4.25), 7.462 (4.02), 7.473 (4.09), 7.485 (4.61), 7.492 (6.04), 7.515 (5.84), 8.304 (14.79), 8.339 (4.51), 8.343 (4.73), 8.362 (4.32), 8.366 (4.26), 8.478 (3.30), 8.497 (3.23), 8.786 (3.90), 8.790 (4.12), 8.798 (3.98), 8.801 (3.72).

### Example 266

### N-[(trans)-2-(2-chloro-4-fluoro-5-methyl phenyl)-1-oxo-2-azaspiro[4.5]dec-8-yl]-5,7-dimethylpyrazolo[1,5-a]pyrimidine-3-carboxamide

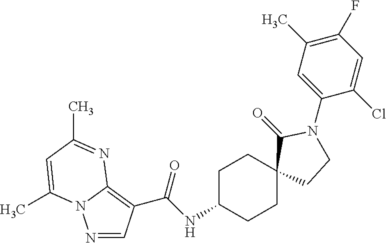 [SMILES: Cc1cc(C)n2ncc(C(=O)N[C@H]3CC[C@@]4(CCN(c5cc(C)c(F)cc5Cl)C4=O)CC3)c2n1]

Benzotriazol-1-yl-oxytripyrrolidinophosphonium hexafluorophosphate (251 mg, 483 μmol) and N,N-diisopropylethylamine (310 μl, 1.8 mmol) were added to a mixture of 8-amino-2-(2-chloro-4-fluoro-5-methylphenyl)-2-azaspiro[4.5]decan-1-one (isomer 1) (150 mg, 483 μmol) and 5,7-dimethylpyrazolo[1,5-a]pyrimidine-3-carboxylic acid (88.3 mg, 439 μmol, Cas No 90349-23-8) in N,N-dimethylformamide (4.5 ml) and the mixture was stirred at room temperature for 20 h. For work-up, water (35 ml) was added and the precipitate formed was collected by filtration. The crude product was recrystallized from acetonitrile to give the title compound (114 mg).

LC-MS (Method 2): Rₜ=1.27 min; MS (ESIpos): m/z=484.1 [M+H]⁺

¹H-NMR (400 MHz, DMSO-d6) δ[ppm]: 1.672 (2.68), 1.681 (3.36), 2.004 (1.11), 2.075 (0.70), 2.143 (1.61), 2.160 (3.12), 2.177 (1.70), 2.224 (6.52), 2.229 (6.51), 2.632 (16.00), 2.737 (9.38), 2.739 (9.61), 3.595 (1.72), 3.613 (3.04), 3.629 (1.63), 7.126 (3.10), 7.129 (3.23), 7.375 (1.70), 7.396 (1.65), 7.491 (2.44), 7.514 (2.42), 8.012 (1.35), 8.031 (1.34), 8.496 (6.82).

### Example 267

### N-[(trans)-2-(2-chloro-4-fluoro-5-methyl phenyl)-1-oxo-2-azaspiro[4.5]dec-8-yl]pyrazolo[1,5-a]pyrimidine-3-carboxamide

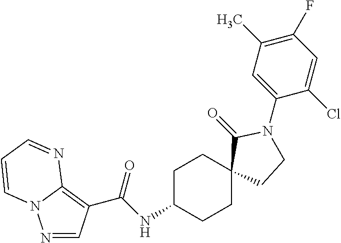 [SMILES: Cc1cc(N2CC[C@]3(CC[C@@H](NC(=O)c4cnn5cccnc45)CC3)C2=O)c(Cl)cc1F]

Benzotriazol-1-yl-oxytripyrrolidinophosphonium hexafluorophosphate (251 mg, 483 μmol) and N,N-diisopropylethylamine (310 μl, 1.8 mmol) were added to a mixture of 8-amino-2-(2-chloro-4-fluoro-5-methylphenyl)-2-azaspiro[4.5]decan-1-one (isomer 1) (150 mg, 483 μmol) and pyrazolo[1,5-a]pyrimidine-3-carboxylic acid (73.8 mg, 439 μmol, Cas No 25940-35-6) in N,N-dimethylformamide (4.5 ml) and the mixture was stirred at room temperature for 20 h. For work-up, water (50 ml) was added and the precipitate formed was collected by filtration, washed with water and dried to give the title compound (127 mg).

LC-MS (Method 2): Rₜ=1.14 min; MS (ESIpos): m/z=456.3 [M+H]⁺

¹H-NMR (400 MHz, DMSO-d6) δ[ppm]: 1.463 (1.65), 1.478 (1.91), 1.492 (2.20), 1.507 (2.12), 1.663 (7.21), 1.672 (8.23), 1.689 (4.77), 1.965 (2.71), 1.988 (2.65), 2.138 (4.04), 2.155 (7.72), 2.172 (4.38), 2.223 (16.00), 2.228 (15.99), 2.522 (4.73), 2.729 (1.31), 2.888 (1.69), 3.591 (4.30), 3.608 (7.64), 3.625 (4.21), 3.831 (1.24), 3.851 (1.23), 7.261 (4.13), 7.272 (3.90), 7.279 (3.98), 7.289 (4.19), 7.371 (4.06), 7.390 (4.14), 7.490 (5.98), 7.514 (5.94), 7.815 (3.50), 7.834 (3.43), 8.579 (15.54), 8.820 (4.40), 8.824 (5.14), 8.830 (4.81), 8.835 (4.51), 9.311 (4.76), 9.315 (4.84), 9.328 (4.77), 9.333 (4.77).

### Example 268

### N-[(trans)-2-(3-chlorophenyl)-1-oxo-2-azaspiro[4.5]dec-8-yl]pyrazolo[1,5-a]pyrimidine-3-carboxamide

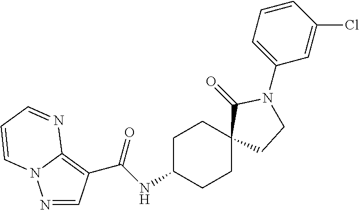 [SMILES: O=C(N[C@H]1CC[C@@]2(CCN(c3cccc(Cl)c3)C2=O)CC1)c1cnn2cccnc12]

Benzotriazol-1-yl-oxytripyrrolidinophosphonium hexafluorophosphate (272 mg, 523 μmol) and N,N-diisopropylethylamine (330 μl, 1.9 mmol) were added to a mixture of 8-amino-2-(3-chlorophenyl)-2-azaspiro[4.5]decan-1-one (isomer 1) (146 mg, 523 μmol) and pyrazolo[1,5-a]pyrimidine-3-carboxylic acid (80.0 mg, 476 μmol, Cas No 25940-35-6) in N,N-dimethylformamide (4.8 ml) and the mixture was stirred at room temperature for 24 h. For work-up, the reaction mixture was concentrated under reduced pressure. The crude product was purified by flash chromatography (2×) (25 g Isolera-cartridge dichloromethane/methanol-gradient 0%->10% methanol) to give after recrystallization from methanol the title compound (129.2 mg).

LC-MS (Method 2): Rₜ=1.15 min; MS (ESIpos): m/z=424.2 [M+H]⁺

¹H-NMR (400 MHz, DMSO-d6) δ[ppm]: 1.458 (1.90), 1.470 (2.00), 1.489 (2.25), 1.500 (2.26), 1.519 (1.07), 1.531 (0.99), 1.626 (0.91), 1.635 (1.02), 1.658 (7.63), 1.684 (2.40), 1.691 (2.75), 1.942 (2.38), 1.950 (2.52), 1.962 (1.53), 1.974 (2.34), 1.983 (2.12), 2.086 (3.88), 2.104 (7.16), 2.121 (4.11), 2.523 (1.15), 2.727 (0.97), 2.888 (1.24), 3.794 (4.28), 3.802 (1.45), 3.811 (7.57), 3.828 (4.77), 3.835 (1.66), 3.846 (1.07), 3.855 (1.22), 3.865 (0.94), 5.759 (9.05), 7.184 (2.27), 7.186 (2.54), 7.189 (2.56), 7.191 (2.41), 7.204 (2.86), 7.206 (3.13), 7.209 (3.18), 7.211 (2.98), 7.259 (4.03), 7.270 (3.99), 7.277 (4.03), 7.287 (4.17), 7.389 (3.73), 7.410 (6.78), 7.430 (3.76), 7.580 (2.57), 7.582 (3.01), 7.585 (2.86), 7.588 (2.80), 7.601 (2.16), 7.603 (2.29), 7.606 (2.45), 7.608 (2.13), 7.811 (3.43), 7.831 (3.34), 7.909 (4.02), 7.914 (7.48), 7.919 (3.93), 8.580 (16.00), 8.817 (4.68), 8.821 (4.88), 8.828 (4.64), 8.831 (4.41), 9.309 (4.72), 9.312 (4.80), 9.326 (4.69), 9.330 (4.66).

### Example 269

### N-[(trans)-2-(3-chlorophenyl)-1-oxo-2-azaspiro[4.5]dec-8-yl]imidazo[1,2-b]pyridazine-3-carboxamide

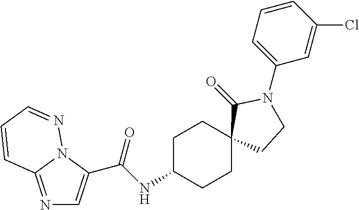 [SMILES: O=C(N[C@H]1CC[C@@]2(CCN(c3cccc(Cl)c3)C2=O)CC1)c1cnc2cccnn12]

Benzotriazol-1-yl-oxytripyrrolidinophosphonium hexafluorophosphate (267 mg, 512 μmol) and N,N-diisopropylethylamine (320 μl, 1.9 mmol) were added to a mixture of imidazo[1,2-b]pyridazine-3-carboxylic acid (80.0 mg, 466 μmol, Cas No 1308384-58-8) and 8-amino-2-(3-chlorophenyl)-2-azaspiro[4.5]decan-1-one (isomer 1) (143 mg, 512 μmol) in N,N-dimethylformamide (4.7 ml) and the mixture was stirred at room temperature for 12 h. For work-up, the mixture was concentrated and was washed with water, dichloromethane and methanol. The residue was purified by preparative HPLC followed by recrystallization from acetonitrile to give the title compound (156.6 mg).

LC-MS (Method 6): Rₜ=1.05 min; MS (ESIpos): m/z=424.2 [M+H]⁺

¹H-NMR (400 MHz, DMSO-d₆): δ [ppm]=8.76 (d, 1H), 8.40 (br d, 1H), 8.34-8.22 (m, 2H), 7.89 (s, 1H), 7.59 (br d, 1H), 7.50-7.32 (m, 2H), 7.19 (br d, 1H), 3.93 (br s, 1H), 3.83 (t, 2H), 2.14 (br t, 2H), 2.05 (br d, 2H), 1.82-1.49 (m, 7H).

### Example 270

### N-[(trans)-2-(3-chlorophenyl)-1-oxo-2-azaspiro[4.5]dec-8-yl]-5,7-dimethylpyrazolo[1,5-a]pyrimidine-3-carboxamide

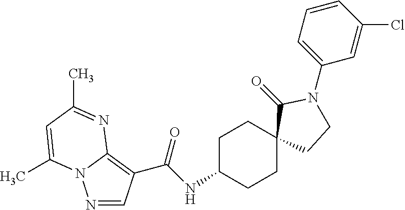 [SMILES: Cc1cc(C)n2ncc(C(=O)N[C@H]3CC[C@@]4(CCN(c5cccc(Cl)c5)C4=O)CC3)c2n1]

Benzotriazol-1-yl-oxytripyrrolidinophosphonium hexafluorophosphate (228 mg, 437 μmol) and N,N-diisopropylethylamine (280 μl, 1.6 mmol) were added to a mixture of 8-amino-2-(3-chlorophenyl)-2-azaspiro[4.5]decan-1-one (isomer 1) (122 mg, 437 μmol) and 5,7-dimethylpyrazolo[1,5-a]pyrimidine-3-carboxylic acid (80.0 mg, 398 μmol, Cas No 90349-23-8) in N,N-dimethylformamide (4 ml) and the mixture was stirred at room temperature for 24 h. For work-up, the mixture was concentrated and was washed with water, dichloromethane and methanol. The residue was recrystallization from acetonitrile to give the title compound (125.7 mg).

LC-MS (Method 6): Rₜ=1.22 min; MS (ESIpos): m/z=452.2 [M+H]⁺

¹H-NMR (400 MHz, DMSO-d6) δ[ppm]: 1.470 (1.28), 1.478 (1.22), 1.508 (1.41), 1.530 (0.65), 1.539 (0.58), 1.651 (1.07), 1.685 (2.50), 1.713 (1.60), 1.721 (1.52), 1.745 (1.73), 1.753 (1.64), 1.777 (0.55), 2.037 (1.53), 2.047 (1.49), 2.069 (1.41), 2.107 (2.15), 2.125 (3.88), 2.143 (2.17), 2.635 (16.00), 2.753 (12.55), 3.046 (9.59), 3.800 (2.48), 3.818 (4.43), 3.835 (2.73), 3.848 (0.75), 3.858 (0.63), 3.867 (0.69), 3.877 (0.53), 7.059 (4.35), 7.162 (1.41), 7.167 (1.34), 7.181 (1.66), 7.187 (1.63), 7.373 (1.48), 7.393 (2.92), 7.414 (1.51), 7.567 (1.69), 7.572 (1.63), 7.588 (1.35), 7.593 (1.32), 7.875 (2.26), 7.880 (3.56), 7.885 (1.84), 7.945 (1.30), 7.962 (1.30), 8.459 (5.71).

### Example 271

### N-[(trans)-2-(2-chlorophenyl)-1-oxo-2-azaspiro[4.5]dec-8-yl]imidazo[1,2-b]pyridazine-3-carboxamide

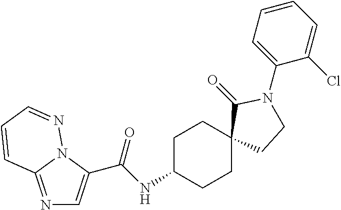 [SMILES: O=C(N[C@H]1CC[C@@]2(CCN(c3ccccc3Cl)C2=O)CC1)c1cnc2cccnn12]

Benzotriazol-1-yl-oxytripyrrolidinophosphonium hexafluorophosphate (187 mg, 359 μmol) and N,N-diisopropylethylamine (230 μl, 1.3 mmol) were added to a mixture of imidazo[1,2-b]pyridazine-3-carboxylic acid (54.8 mg, 326 μmol Cas No, 1308384-58-8) and 8-amino-2-(2-chlorophenyl)-2-azaspiro[4.5]decan-1-one (isomer 1) (100 mg, 359 μmol) in N,N-dimethylformamide (3.3 ml) and the mixture was stirred at room temperature for 20 h. For work-up, water (50 ml) was added and the precipitated formed was collected by filtration, washed with water and dried at 50° C. to give the title compound (128 mg).

LC-MS (Method 2): Rₜ=0.99 min; MS (ESIpos): m/z=424.5 [M+H]⁺

¹H-NMR (400 MHz, DMSO-d6) δ [ppm]: 1.526 (1.57), 1.548 (2.75), 1.567 (2.35), 1.598 (1.25), 1.688 (8.62), 1.696 (9.83), 1.712 (6.17), 1.987 (3.25), 2.009 (2.94), 2.157 (4.94), 2.174 (9.25), 2.191 (5.10), 3.637 (5.25), 3.655 (9.19), 3.672 (4.95), 3.886 (1.50), 3.905 (1.45), 7.364 (1.14), 7.374 (2.32), 7.381 (1.23), 7.386 (3.38), 7.392 (2.06), 7.395 (2.41), 7.405 (7.64), 7.411 (12.62), 7.414 (13.88), 7.423 (5.32), 7.428 (2.06), 7.451 (4.63), 7.463 (4.46), 7.475 (4.52), 7.486 (4.68), 7.561 (3.28), 7.564 (5.32), 7.578 (2.58), 7.582 (4.14), 7.585 (2.74), 8.305 (16.00), 8.339 (4.76), 8.343 (5.14), 8.362 (4.84), 8.366 (4.81), 8.482 (3.79), 8.501 (3.72), 8.789 (4.41), 8.793 (4.57), 8.800 (4.61), 8.804 (4.32).

### Example 272

### N-[(trans)-2-(2-chlorophenyl)-1-oxo-2-azaspiro[4.5]dec-8-yl]-1H-pyrazolo[4,3-b]pyridine-3-carboxamide

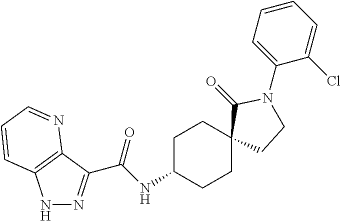 [SMILES: O=C(N[C@H]1CC[C@@]2(CCN(c3ccccc3Cl)C2=O)CC1)c1n[nH]c2cccnc12]

Benzotriazol-1-yl-oxytripyrrolidinophosphonium hexafluorophosphate (153 mg, 294 μmol) and N,N-diisopropylethylamine (190 μl, 1.1 mmol) were added to a mixture of 1H-pyrazolo[4,3-b]pyridine-3-carboxylic acid (45.0 mg, 268 μmol, Cas No 1260648-73-4) and 8-amino-2-(2-chlorophenyl)-2-azaspiro[4.5]decan-1-one (isomer 1) (82.1 mg, 294 μmol) in N,N-dimethylformamide (2.7 ml) and the mixture was stirred at room temperature for 24 h. For work-up, the reaction mixture was concentrated and the residue was purified by flash chromatography (25 g Isolera-cartridge dichloromethane/methanol-gradient 0%->10% methanol) The crude product was recrystallized from dichloromethane to give the title compound (59.4 mg).

LC-MS (Method 1): Rₜ=1.02 min; MS (ESIpos): m/z=424.1 [M+H]⁺

¹H-NMR (400 MHz, DMSO-d6) δ[ppm]: 1.488 (0.72), 1.519 (1.89), 1.532 (2.34), 1.545 (2.87), 1.561 (2.69), 1.575 (1.62), 1.592 (1.27), 1.693 (9.80), 1.702 (11.00), 1.719 (6.74), 1.994 (3.44), 2.003 (3.58), 2.026 (3.33), 2.035 (3.03), 2.162 (5.39), 2.179 (10.36), 2.196 (5.72), 2.318 (0.64), 2.323 (0.90), 2.327 (0.64), 2.660 (0.66), 2.665 (0.89), 2.670 (0.63), 3.156 (1.36), 3.166 (1.38), 3.632 (5.78), 3.650 (10.33), 3.667 (5.59), 3.867 (0.67), 3.886 (1.33), 3.896 (1.64), 3.905 (1.41), 3.915 (1.64), 3.924 (1.25), 5.756 (3.59), 7.361 (1.30), 7.372 (3.42), 7.378 (1.38), 7.384 (3.82), 7.390 (2.84), 7.392 (2.90), 7.402 (7.78), 7.409 (14.36), 7.412 (16.00), 7.421 (6.62), 7.425 (2.86), 7.482 (4.25), 7.492 (4.17), 7.503 (4.31), 7.514 (4.43), 7.558 (3.90), 7.561 (5.68), 7.576 (3.04), 7.580 (5.00), 7.583 (2.92), 8.162 (3.42), 8.183 (3.24), 8.634 (1.96), 8.652 (1.94), 8.682 (5.21), 8.685 (5.43), 8.694 (5.30), 8.697 (4.74), 13.897 (0.55).

### Example 273

### N-[(trans)-2-(2-chlorophenyl)-1-oxo-2-azaspiro[4.5]dec-8-yl]-7-methylpyrazolo[1,5-a]pyrimidine-3-carboxamide

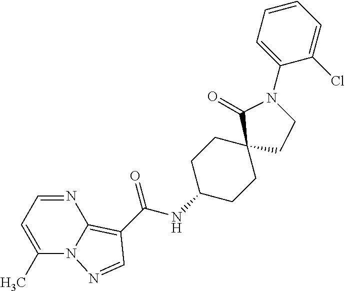 [SMILES: Cc1ccnc2c(C(=O)N[C@H]3CC[C@@]4(CCN(c5ccccc5Cl)C4=O)CC3)cnn12]

Benzotriazol-1-yl-oxytripyrrolidinophosphonium hexafluorophosphate (138 mg, 265 μmol) and N,N-diisopropylethylamine (170 μl, 970 μmol) were added to a mixture of 7-methylpyrazolo[1,5-a]pyrimidine-3-carboxylic acid (45.0 mg, 241 μmol) and 8-amino-2-(2-chlorophenyl)-2-azaspiro[4.5]decan-1-one (isomer 1) (74.0 mg, 265 μmol) in N,N-dimethylformamide (2.5 ml) and the mixture was stirred at room temperature for 24 h. For work-up, the reaction mixture was concentrated and the residue was purified by flash chromatography (25 g Isolera-cartridge dichloromethane/methanol-gradient 0%->10% methanol) The crude product was recrystallized from diethyl ether to give the title compound (68.3 mg).

LC-MS (Method 1): Rₜ=1.07 min; MS (ESIpos): m/z=438.3 [M+H]⁺

¹H-NMR (400 MHz, DMSO-d6) δ[ppm]: 1.463 (0.98), 1.487 (1.42), 1.504 (1.31), 1.535 (0.64), 1.674 (4.95), 1.682 (5.53), 1.691 (3.18), 1.699 (3.61), 1.707 (3.12), 1.723 (1.65), 1.732 (0.83), 1.740 (0.77), 1.962 (1.75), 1.971 (1.82), 1.993 (1.70), 2.003 (1.54), 2.152 (2.81), 2.169 (5.27), 2.186 (2.87), 2.323 (0.50), 2.665 (0.57), 2.801 (16.00), 2.993 (0.58), 3.000 (1.02), 3.009 (1.00), 3.016 (0.57), 3.625 (3.00), 3.642 (5.24), 3.660 (2.83), 3.821 (0.69), 3.831 (0.85), 3.841 (0.72), 3.850 (0.82), 3.860 (0.65), 5.755 (0.85), 7.231 (2.70), 7.233 (2.75), 7.242 (2.63), 7.244 (2.74), 7.358 (0.66), 7.369 (1.32), 7.376 (0.73), 7.381 (2.02), 7.386 (1.18), 7.389 (1.35), 7.395 (1.49), 7.399 (4.73), 7.404 (7.35), 7.407 (8.06), 7.414 (2.56), 7.417 (3.00), 7.421 (1.17), 7.554 (1.91), 7.558 (3.11), 7.561 (1.89), 7.572 (1.57), 7.576 (2.46), 7.579 (1.54), 7.897 (2.29), 7.917 (2.24), 8.593 (9.94), 8.713 (4.88), 8.724 (4.47).

### Example 274

### N-[(trans)-2-(2-chlorophenyl)-1-oxo-2-azaspiro[4.5]dec-8-yl]-1H-pyrrolo[3,2-b]pyridine-3-carboxamide

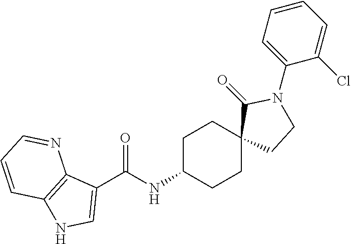 [SMILES: O=C(N[C@H]1CC[C@@]2(CCN(c3ccccc3Cl)C2=O)CC1)c1c[nH]c2cccnc12]

Benzotriazol-1-yl-oxytripyrrolidinophosphonium hexafluorophosphate (154 mg, 296 μmol) and N,N-diisopropylethylamine (190 μl, 1.1 mmol) were added to a mixture of 1H-pyrrolo[3,2-b]pyridine-3-carboxylic acid (45.0 mg, 269 μmol, Cas No 860496-20-4) and trans-8-amino-2-(2-chlorophenyl)-2-azaspiro[4.5]decan-1-one 8-amino-2-(2-chlorophenyl)-2-azaspiro[4.5]decan-1-one (isomer 1) (75.0 mg, 269 μmol) in N,N-dimethylformamide (2.7 ml) and the mixture was stirred at room temperature for 7 h. For work-up, the reaction mixture was concentrated and the residue was purified by flash chromatography (25 g Isolera-cartridge dichloromethane/methanol-gradient 0%->10% methanol) The crude product was recrystallized from methanol to give the title compound (44.4 mg).

LC-MS (Method 1): Rₜ=0.90 min; MS (ESIpos): m/z=423.3 [M+H]⁺

¹H-NMR (400 MHz, DMSO-d6) δ[ppm]: 1.427 (0.69), 1.458 (1.74), 1.470 (2.01), 1.484 (2.44), 1.500 (2.25), 1.513 (1.42), 1.530 (1.13), 1.688 (8.53), 1.697 (9.56), 1.705 (5.36), 1.714 (6.23), 1.720 (4.69), 2.000 (3.07), 2.008 (3.17), 2.019 (2.04), 2.031 (2.94), 2.041 (2.67), 2.169 (5.22), 2.186 (9.74), 2.197 (1.43), 2.204 (5.47), 2.323 (0.52), 2.327 (0.73), 2.332 (0.53), 2.523 (1.51), 2.665 (0.52), 2.669 (0.72), 2.673 (0.54), 3.159 (0.47), 3.172 (0.55), 3.634 (5.66), 3.641 (1.55), 3.651 (9.70), 3.660 (1.47), 3.668 (5.36), 3.815 (0.62), 3.825 (0.70), 3.834 (1.21), 3.844 (1.50), 3.853 (1.26), 3.863 (1.45), 3.872 (1.14), 3.891 (0.55), 7.238 (5.12), 7.250 (4.91), 7.259 (5.03), 7.270 (5.25), 7.363 (1.31), 7.375 (3.81), 7.381 (1.28), 7.386 (4.13), 7.392 (2.75), 7.394 (2.64), 7.405 (7.62), 7.412 (13.02), 7.414 (16.00), 7.424 (6.15), 7.428 (2.72), 7.561 (3.71), 7.565 (5.57), 7.567 (3.79), 7.578 (2.92), 7.582 (5.12), 7.585 (3.08), 7.906 (6.18), 7.910 (6.16), 7.927 (5.77), 7.931 (5.81), 8.166 (9.57), 8.174 (9.28), 8.478 (5.84), 8.482 (6.16), 8.490 (5.97), 8.493 (5.37), 8.718 (4.30), 8.737 (4.14), 11.949 (2.78).

### Example 275

### N-[(trans)-2-(3-chlorophenyl)-1-oxo-2-azaspiro[4.5]dec-8-yl]-1H-pyrrolo[3,2-b]pyridine-3-carboxamide

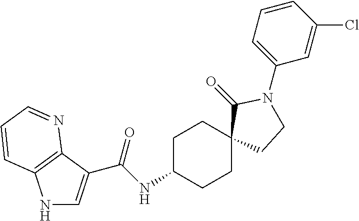 [SMILES: O=C(N[C@H]1CC[C@@]2(CCN(c3cccc(Cl)c3)C2=O)CC1)c1c[nH]c2cccnc12]

Benzotriazol-1-yl-oxytripyrrolidinophosphonium hexafluorophosphate (154 mg, 296 μmol) and N,N-diisopropylethylamine (190 μl, 1.1 mmol) were added to a mixture of 1H-pyrrolo[3,2-b]pyridine-3-carboxylic acid (45.0 mg, 269 μmol, Cas No 860496-20-4) and 8-amino-2-(3-chlorophenyl)-2-azaspiro[4.5]decan-1-one (isomer 1) (75.0 mg, 269 μmol) in N,N-dimethylformamide (2.7 ml) and the mixture was stirred at room temperature for 7 h. For work-up, the reaction mixture was concentrated and the residue was purified by flash chromatography (25 g Isolera-cartridge dichloromethane/methanol-gradient 0%->10% methanol) The crude product was recrystallized from diethyl ether to give the title compound (79.1 mg).

LC-MS (Method 1): Rₜ=1.01 min; MS (ESIpos): m/z=423.2 [M+H]⁺

¹H-NMR (400 MHz, DMSO-d6) δ[ppm]: 1.088 (0.47), 1.220 (2.56), 1.238 (13.85), 1.253 (16.00), 1.268 (9.32), 1.446 (0.57), 1.458 (0.60), 1.476 (0.66), 1.487 (0.66), 1.668 (2.28), 1.695 (0.85), 1.703 (0.92), 1.711 (0.99), 1.720 (1.02), 1.728 (2.21), 1.735 (1.13), 1.744 (0.87), 1.990 (0.79), 2.013 (0.73), 2.104 (1.00), 2.121 (1.86), 2.139 (1.05), 2.522 (1.21), 2.988 (0.52), 2.998 (0.88), 3.005 (1.42), 3.014 (1.45), 3.021 (0.83), 3.031 (0.49), 3.117 (0.50), 3.125 (1.23), 3.136 (1.29), 3.143 (1.28), 3.154 (1.22), 3.162 (0.51), 3.594 (0.77), 3.604 (0.85), 3.611 (1.07), 3.620 (1.06), 3.627 (0.85), 3.637 (0.75), 3.799 (1.10), 3.816 (1.99), 3.833 (1.27), 7.188 (0.71), 7.192 (0.73), 7.209 (0.86), 7.212 (0.87), 7.238 (0.71), 7.249 (0.72), 7.258 (0.77), 7.270 (0.74), 7.392 (0.79), 7.412 (1.46), 7.432 (0.78), 7.585 (0.83), 7.589 (0.83), 7.606 (0.65), 7.610 (0.69), 7.906 (0.96), 7.909 (1.01), 7.919 (1.20), 7.924 (2.13), 7.929 (1.87), 8.166 (1.56), 8.174 (1.61), 8.473 (1.04), 8.476 (1.10), 8.484 (1.03), 8.488 (0.98), 8.711 (0.88), 8.731 (0.86), 11.951 (0.64).

### Example 276

### N⁵-[(trans)-2-(3-chlorophenyl)-1-oxo-2-azaspiro[4.5]dec-8-yl]-N⁴-methyl-1H-imidazole-4,5-dicarboxamide

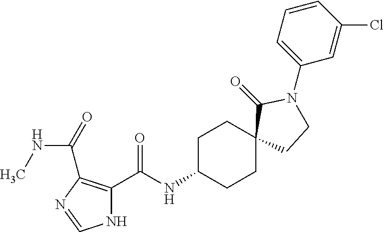 [SMILES: CNC(=O)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol), methanamine in THF (760 μl, 2.0 M, 1.5 mmol, Cas No 74-89-5) in tetrahydrofuran (4.9 ml) was stirred for 1 d at room temperature. For work-up, the reaction mixture was concentrated and the residue was purified by flash chromatography (24 g Snap-cartridge, dichloromethane/methanol-gradient, 0%->10% methanol). The obtained product was recrystallized from diethyl ether give the title compound (91.3 mg).

LC-MS (Method 1): Rₜ=1.11 min; MS (ESIpos): m/z=430.2 [M+H]⁺

¹H-NMR (400 MHz, DMSO-d6) δ[ppm]: 1.071 (2.91), 1.088 (5.69), 1.105 (2.96), 1.229 (0.52), 1.334 (0.83), 1.364 (2.29), 1.376 (2.57), 1.392 (2.88), 1.407 (2.60), 1.422 (1.48), 1.437 (1.17), 1.620 (12.68), 1.641 (11.06), 1.651 (12.26), 1.667 (6.47), 1.677 (4.73), 1.763 (3.04), 1.960 (3.58), 1.983 (3.32), 2.045 (2.52), 2.068 (6.57), 2.085 (9.01), 2.103 (4.55), 2.323 (1.14), 2.327 (1.53), 2.332 (1.17), 2.523 (6.16), 2.665 (1.22), 2.669 (1.58), 2.673 (1.25), 2.794 (15.90), 2.807 (16.00), 2.836 (9.19), 2.848 (8.86), 3.353 (1.51), 3.371 (2.94), 3.388 (2.78), 3.406 (0.94), 3.706 (1.27), 3.717 (1.61), 3.734 (1.61), 3.746 (1.35), 3.791 (6.94), 3.808 (11.43), 3.826 (6.39), 7.185 (5.09), 7.189 (5.32), 7.205 (6.23), 7.209 (6.39), 7.388 (5.97), 7.409 (10.94), 7.429 (5.90), 7.579 (5.95), 7.598 (4.94), 7.808 (11.35), 7.819 (6.42), 7.901 (4.18), 7.906 (3.48), 7.912 (5.14), 7.918 (7.53), 7.923 (4.36), 8.401 (2.10), 8.423 (2.03), 8.622 (2.88), 8.635 (2.86), 11.050 (1.51), 11.061 (1.45), 11.262 (3.87), 11.281 (3.79), 13.206 (4.26), 13.253 (1.22).

### Example 277

### N⁵-[(trans)-2-(3-chlorophenyl)-1-oxo-2-azaspiro[4.5]dec-8-yl]-N⁴-ethyl-1H-imidazole-4,5-dicarboxamide

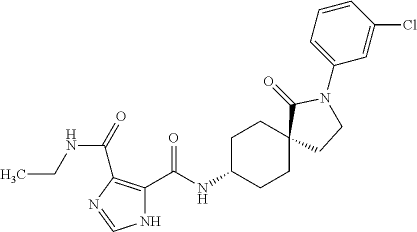 [SMILES: CCNC(=O)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol), ethanamine in THF (760 μl, 2.0 M, 1.5 mmol, Cas No 75-04-7) in tetrahydrofuran (4.9 ml) was stirred for 1 d at room temperature. For work-up, the reaction mixture was concentrated and the residue was purified by flash chromatography (24 g Snap-cartridge, dichloromethane/methanol-gradient, 0%->10% methanol). The obtained product was recrystallized from diethyl ether give the title compound (40 mg).

LC-MS (Method 1): Rₜ=1.18 min; MS (ESIpos): m/z=444.2 [M+H]⁺

¹H-NMR (400 MHz, DMSO-d6) δ[ppm]: 1.099 (7.47), 1.116 (16.00), 1.129 (8.09), 1.135 (9.04), 1.147 (12.95), 1.165 (6.44), 1.363 (1.79), 1.376 (2.04), 1.392 (2.31), 1.406 (2.11), 1.421 (1.25), 1.436 (1.01), 1.624 (13.57), 1.639 (10.18), 1.650 (10.42), 1.666 (5.75), 1.676 (4.50), 1.765 (2.73), 1.952 (2.68), 1.960 (2.85), 1.985 (2.73), 1.992 (2.53), 2.046 (2.70), 2.064 (6.88), 2.085 (8.21), 2.102 (4.10), 2.322 (1.08), 2.326 (1.43), 2.331 (1.11), 2.522 (11.06), 2.664 (1.11), 2.668 (1.47), 2.673 (1.13), 3.270 (1.18), 3.288 (4.15), 3.302 (7.03), 3.315 (5.43), 3.320 (7.62), 3.351 (2.19), 3.705 (1.08), 3.715 (1.30), 3.733 (1.33), 3.742 (1.11), 3.789 (5.78), 3.807 (10.18), 3.824 (5.97), 7.183 (4.42), 7.188 (4.69), 7.204 (5.53), 7.208 (5.65), 7.387 (5.68), 7.407 (10.40), 7.428 (5.68), 7.574 (5.09), 7.579 (5.21), 7.595 (4.18), 7.600 (4.28), 7.806 (8.90), 7.816 (6.81), 7.898 (2.88), 7.903 (4.99), 7.911 (5.04), 7.917 (6.71), 7.922 (3.81), 8.391 (2.41), 8.413 (2.41), 8.637 (1.52), 8.652 (2.95), 8.668 (1.57), 11.123 (1.13), 11.136 (2.04), 11.150 (1.13), 11.245 (3.32), 11.262 (3.24), 13.205 (3.96), 13.229 (1.70).

### Example 278

### N⁵-[(trans)-2-(3-chlorophenyl)-1-oxo-2-azaspiro[4.5]dec-8-yl]-N⁴-(propan-2-yl)-1H-imidazole-4,5-dicarboxamide

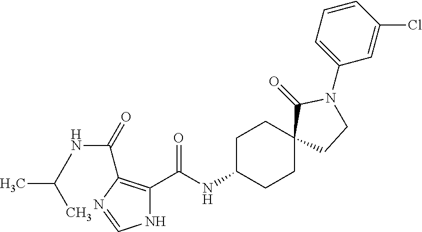 [SMILES: CC(C)NC(=O)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol), propan-2-amine (130 μl, 1.5 mmol Cas No 75-31-0) in tetrahydrofuran (4.9 ml) was stirred for 3 h at room temperature. For work-up, the reaction mixture was concentrated and the residue was purified by flash chromatography (24 g Snap-cartridge, dichloromethane/methanol-gradient, 0%->10% methanol). The obtained product was recrystallized from diethyl ether give the title compound (48.3 mg).

LC-MS (Method 1): Rₜ=1.24 min; MS (ESIpos): m/z=458.3 [M+H]⁺

¹H-NMR (400 MHz, DMSO-d6) δ[ppm]: 1.071 (0.56), 1.088 (1.20), 1.105 (0.65), 1.176 (15.63), 1.192 (16.00), 1.378 (0.56), 1.392 (0.63), 1.407 (0.58), 1.625 (5.57), 1.650 (3.20), 1.763 (1.33), 1.962 (0.76), 1.985 (0.75), 2.048 (1.16), 2.066 (2.83), 2.084 (2.68), 2.103 (1.08), 3.370 (0.75), 3.388 (0.59), 3.789 (1.75), 3.803 (3.15), 3.820 (2.06), 3.985 (0.45), 4.003 (0.73), 4.019 (0.71), 4.036 (0.45), 7.188 (1.50), 7.209 (1.76), 7.387 (1.64), 7.407 (3.01), 7.428 (1.68), 7.579 (1.78), 7.599 (1.50), 7.806 (2.76), 7.811 (3.24), 7.901 (1.20), 7.907 (2.17), 7.912 (2.19), 7.917 (1.90), 7.922 (1.10), 8.285 (0.76), 8.306 (0.73), 8.376 (1.03), 8.399 (1.04), 11.087 (0.88), 11.104 (0.88), 11.208 (0.83), 11.227 (0.84), 13.219 (1.41).

### Example 279

### N⁵-[(trans)-2-(3-chlorophenyl)-1-oxo-2-azaspiro[4.5]dec-8-yl]-N⁴-cyclopropyl-1H-imidazole-4,5-dicarboxamide

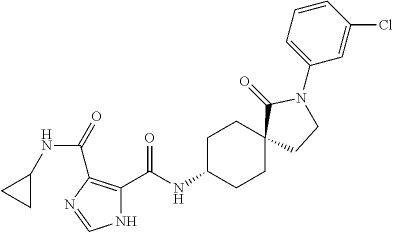 [SMILES: O=C(NC1CC1)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol), cyclopropanamine (110 μl, 1.5 mmol Cas No 765-30-0) in tetrahydrofuran (4.9 ml) was stirred for 1 d at room temperature. For work-up, the reaction mixture was concentrated and the residue was purified by flash chromatography (dichloromethane/methanol-gradient, 0%->10% methanol). The obtained product was recrystallized from diethyl ether give the title compound (78.1 mg).

LC-MS (Method 1): Rₜ=1.17 min; MS (ESIPOS): m/z=456.2

¹H-NMR (400 MHz, DMSO-d6) δ[ppm]: 0.505 (5.06), 0.682 (11.40), 0.755 (4.60), 0.770 (4.41), 1.410 (2.17), 1.620 (16.00), 1.647 (10.12), 1.744 (3.73), 1.966 (2.80), 2.042 (3.06), 2.059 (5.56), 2.077 (5.45), 2.096 (5.21), 2.113 (2.89), 2.326 (1.30), 2.670 (1.32), 2.834 (2.02), 3.800 (8.90), 3.810 (8.81), 7.188 (4.84), 7.207 (5.71), 7.388 (4.15), 7.408 (7.53), 7.428 (4.02), 7.578 (5.71), 7.599 (4.58), 7.794 (5.58), 7.823 (5.84), 7.905 (5.21), 8.404 (2.37), 8.425 (2.32), 8.575 (2.50), 11.183 (2.37), 11.200 (2.37), 11.248 (2.61), 13.233 (3.23).

### Example 280

### N-[(trans)-2-(3-chlorophenyl)-1-oxo-2-azaspiro[4.5]dec-8-yl]-4-[(3-fluoroazetidin-1-yl)carbonyl]-1H-imidazole-5-carboxamide

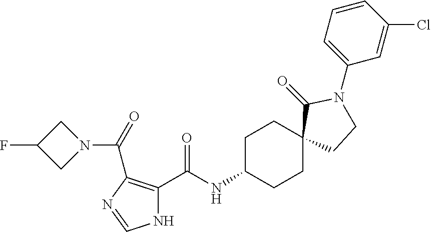 [SMILES: O=C(N[C@H]1CC[C@@]2(CCN(c3cccc(Cl)c3)C2=O)CC1)c1[nH]cnc1C(=O)N1CC(F)C1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol), 3-fluoroazetidine hydrochloride (67.9 mg, 609 μmol, Cas No 617718-46-4) and triethylamine (130 μl) in tetrahydrofuran (4.9 ml) was stirred for 1 d at room temperature. For work-up, the reaction mixture was concentrated and the residue was purified by flash chromatography (dichloromethane/methanol-gradient, 0%->10% methanol). The obtained product was recrystallized from diethyl ether give the title compound (103.7 mg).

LC-MS (Method 1): Rₜ=1.16 min; MS (ESIpos): m/z=474.1 [M+H]⁺

¹H-NMR (400 MHz, DMSO-d₆): δ [ppm]=13.54-12.78 (m, 1H), 11.09 (d, 1H), 7.92 (t, 1H), 7.84 (s, 1H), 7.66-7.54 (m, 1H), 7.41 (t, 1H), 7.28-7.05 (m, 1H), 5.55-5.29 (m, 1H), 5.08-4.83 (m, 1H), 4.70-4.56 (m, 1H), 4.52-4.34 (m, 1H), 4.22-3.99 (m, 1H), 3.87-3.64 (m, 3H), 2.08 (t, 2H), 1.96 (br dd, 2H), 1.73-1.51 (m, 4H), 1.38 (dt, 2H)

### Example 281

### N⁵-[(trans)-2-(3-chlorophenyl)-1-oxo-2-azaspiro[4.5]dec-8-yl]-N⁴-(2,2,2-trifluoroethyl)-1H-imidazole-4,5-dicarboxamide

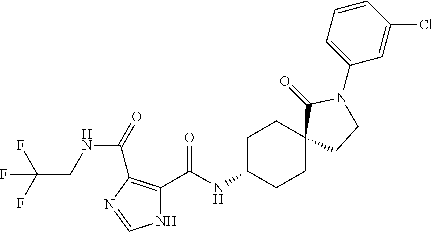 [SMILES: O=C(NCC(F)(F)F)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol), 2,2,2-trifluoroethanamine hydrochloride (247 mg, 1.83 mmol, Cas No 373-88-6) and triethylamine (260 μl) in tetrahydrofuran (4.1 ml) was stirred for 1 d at room temperature, followed by 1 d at 80° C. and 2 d at 130° C. in a sealed tube. For work-up, the reaction mixture was concentrated and the residue was purified by flash chromatography (dichloromethane/methanol-gradient, 0%->10% methanol). The obtained product was recrystallized from diethyl ether give the title compound (37.9 mg).

LC-MS (Method 2): Rₜ=1.20 min; MS (ESIpos): m/z=498.2 [M+H]⁺

¹H-NMR (400 MHz, DMSO-d6) δ[ppm]: 1.071 (1.11), 1.088 (2.19), 1.105 (1.16), 1.391 (1.43), 1.636 (16.00), 1.763 (3.54), 1.965 (1.80), 2.068 (7.34), 2.327 (1.58), 2.669 (1.61), 3.371 (1.72), 3.389 (1.35), 3.406 (0.45), 3.788 (7.45), 3.805 (12.91), 3.822 (7.84), 4.079 (1.69), 4.216 (2.59), 7.185 (4.88), 7.188 (4.96), 7.204 (5.91), 7.208 (5.89), 7.388 (4.94), 7.408 (9.27), 7.428 (5.12), 7.576 (5.49), 7.600 (4.62), 7.907 (8.92), 8.545 (1.95), 8.567 (1.95), 9.157 (1.21), 10.777 (1.16), 11.832 (2.06), 13.396 (1.69), 13.454 (1.98).

### Example 282

### N⁵-[(trans)-2-(3-chlorophenyl)-1-oxo-2-azaspiro[4.5]dec-8-yl]-N⁴-(2-methoxyethyl)-1H-imidazole-4,5-dicarboxamide

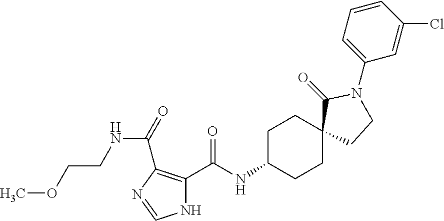 [SMILES: COCCNC(=O)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol) and 2-methoxyethanamine (130 μl, 1.5 mmol, Cas No 109-85-3) in tetrahydrofuran (4.1 ml) was stirred for 1 d at room temperature. For work-up, the reaction mixture was concentrated and the residue was purified by flash chromatography (2×) (dichloromethane/methanol-gradient, 0%->10% methanol, followed dichloromethane/methanol-gradient, 0%->10% methanol). The obtained product was recrystallized from diethyl ether give the title compound (50.8 mg).

LC-MS (Method 6) Rₜ=1.11 min; MS (ESIpos): m/z=474.2 [M+H]⁺

¹H-NMR (400 MHz, DMSO-d6) δ[ppm]: 1.070 (0.92), 1.088 (1.87), 1.105 (0.94), 1.392 (0.59), 1.631 (3.71), 1.763 (0.74), 1.963 (0.75), 1.986 (0.70), 2.066 (1.80), 2.082 (2.06), 2.099 (0.94), 3.274 (16.00), 3.333 (11.88), 3.352 (0.61), 3.370 (0.98), 3.388 (0.94), 3.788 (1.90), 3.805 (3.45), 3.822 (2.06), 7.184 (1.27), 7.188 (1.36), 7.204 (1.58), 7.208 (1.65), 7.387 (1.41), 7.407 (2.65), 7.428 (1.44), 7.579 (1.50), 7.599 (1.25), 7.821 (2.42), 7.915 (1.63), 8.503 (0.62), 11.136 (0.64), 11.153 (0.66), 13.249 (1.48).

### Example 283

### N-[(trans)-2-(3-chlorophenyl)-1-oxo-2-azaspiro[4.5]dec-8-yl]-4-[(4-methylpiperazin-1-yl)carbonyl]-1H-imidazole-5-carboxamide

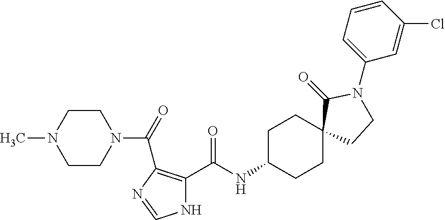 [SMILES: CN1CCN(C(=O)c2nc[nH]c2C(=O)N[C@H]2CC[C@@]3(CCN(c4cccc(Cl)c4)C3=O)CC2)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol) and 1-methylpiperazine (170 μl, 1.5 mmol, Cas No 109-C₁-3) in tetrahydrofuran (4.9 ml) was stirred for 1 d at room temperature. For work-up, the reaction mixture was concentrated and the residue was purified by flash chromatography (dichloromethane/methanol-gradient, 0%->10% methanol). The obtained product was recrystallized from diethyl ether give the title compound (98.1 mg).

LC-MS (Method 6): Rₜ=0.76 min; MS (ESIpos): m/z=499.2 [M+H]⁺

¹H-NMR (400 MHz, CHLOROFORM-d) b [ppm]: 1.155 (0.79), 1.173 (0.40), 1.696 (0.74), 1.705 (0.88), 2.130 (0.73), 2.262 (2.15), 2.412 (0.64), 2.425 (0.69), 2.558 (5.59), 2.563 (11.84), 2.567 (16.00), 2.572 (11.89), 2.576 (5.84), 3.401 (6.10), 3.437 (0.43), 3.849 (0.50), 3.867 (0.88), 3.884 (0.49), 7.452 (0.47), 7.473 (0.87), 7.493 (0.48), 7.868 (0.86), 7.971 (0.51), 7.976 (0.65).

### Example 284

### N⁵-[(trans)-2-(3-chlorophenyl)-1-oxo-2-azaspiro[4.5]dec-8-yl]-N⁴-(2-hydroxy-2-methylpropyl)-1H-imidazole-4,5-dicarboxamide

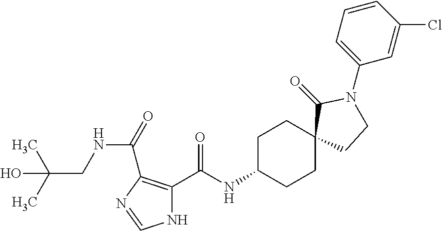 [SMILES: CC(C)(O)CNC(=O)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol) and 1-amino-2-methylpropan-2-ol (71 μl, 1.5 mmol, Cas No 2854-16-2) in tetrahydrofuran (4.9 ml) was stirred for 1 d at room temperature. For work-up, the reaction mixture was concentrated and the residue was purified by flash chromatography (24 g Snap-cartridge, dichloromethane/methanol-gradient, 0%->10% methanol). The obtained product was recrystallized from diethyl ether give the title compound (109.9 mg).

LC-MS (Method 6): Rₜ=1.07 min; MS (ESIpos): m/z=488.2 [M+H]⁺

¹H-NMR (400 MHz, DMSO-d6) δ[ppm]: 1.119 (14.69), 1.125 (16.00), 1.382 (0.54), 1.624 (4.40), 1.638 (2.67), 1.649 (2.50), 1.771 (0.91), 1.962 (0.66), 1.986 (0.63), 2.047 (0.98), 2.057 (1.29), 2.065 (2.05), 2.075 (2.03), 2.082 (1.35), 2.092 (1.04), 2.522 (1.00), 3.258 (2.88), 3.272 (2.77), 3.788 (1.67), 3.805 (2.95), 3.820 (1.63), 4.535 (3.36), 4.752 (3.10), 5.760 (1.11), 7.183 (1.24), 7.185 (1.30), 7.188 (1.27), 7.203 (1.50), 7.206 (1.57), 7.208 (1.48), 7.386 (1.77), 7.407 (3.17), 7.427 (1.72), 7.577 (1.52), 7.597 (1.21), 7.818 (2.49), 7.828 (2.41), 7.898 (0.97), 7.903 (1.78), 7.908 (1.63), 7.915 (1.70), 7.920 (0.89), 8.202 (0.76), 8.355 (0.85), 8.376 (0.82), 11.118 (1.14), 11.135 (1.49), 13.209 (0.62), 13.280 (0.87).

### Example 285

### N⁵-[(trans)-2-(3-chlorophenyl)-1-oxo-2-azaspiro[4.5]dec-8-yl]-N⁴-(1-methoxypropan-2-yl)-1H-imidazole-4,5-dicarboxamide

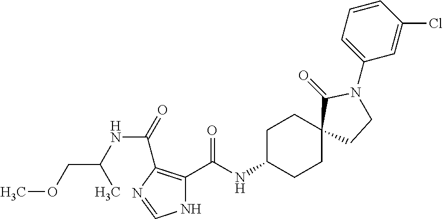 [SMILES: COCC(C)NC(=O)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol) and 1-methoxypropan-2-amine (160 μl, 1.5 mmol, Cas No 37143-54-7) in tetrahydrofuran (4.9 ml) was stirred for 1 d at room temperature. For work-up, the reaction mixture was concentrated and the residue was purified by flash chromatography (dichloromethane/methanol-gradient, 0%->10% methanol). The obtained product was recrystallized from diethyl ether give the title compound (88.4 mg).

LC-MS (Method 6): Rₜ=1.18 min; MS (ESIpos): m/z=488.2 [M+H]⁺

¹H-NMR (400 MHz, DMSO-d6) δ[ppm]: 1.070 (0.99), 1.088 (2.00), 1.105 (1.15), 1.149 (6.29), 1.165 (6.33), 1.394 (0.70), 1.636 (6.72), 1.765 (1.04), 1.966 (0.89), 2.076 (3.08), 2.670 (0.50), 3.333 (16.00), 3.370 (1.54), 3.388 (1.66), 3.405 (1.27), 3.788 (2.92), 3.805 (4.99), 3.822 (2.98), 4.126 (0.49), 7.188 (2.03), 7.207 (2.39), 7.387 (1.75), 7.407 (3.30), 7.428 (1.78), 7.578 (2.34), 7.599 (1.90), 7.819 (5.10), 7.911 (3.44), 8.287 (0.53), 8.400 (0.55), 11.141 (1.08), 13.240 (1.48).

### Example 286

### N-[(trans)-2-(3-chlorophenyl)-1-oxo-2-azaspiro[4.5]dec-8-yl]-4-(morpholin-4-ylcarbonyl)-1H-imidazole-5-carboxamide

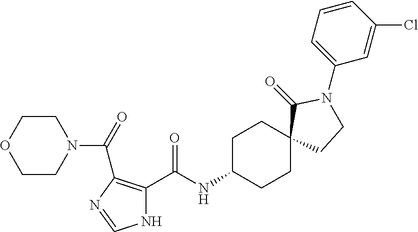 [SMILES: O=C(N[C@H]1CC[C@@]2(CCN(c3cccc(Cl)c3)C2=O)CC1)c1[nH]cnc1C(=O)N1CCOCC1]

A mixture of phenyl 5-{[2-(3-chlorophenyl)-1-oxo-2-azaspiro[4.5]dec-8-yl]carbamoyl}-1H-imidazole-4-carboxylate (isomer 1) (150 mg, 304 μmol) and morpholine (130 μl, 1.5 mmol, Cas No 110-91-8) in tetrahydrofuran (4.9 ml) was stirred for 1 d at room temperature. For work-up, the reaction mixture was concentrated and the residue was purified by flash chromatography (24 g Snap-cartridge, dichloromethane/methanol-gradient, 0%->10% methanol). The obtained product was recrystallized from dichloromethane give the title compound (48.1 mg).

LC-MS (Method 1): Rₜ=1.04 min; MS (ESIpos): m/z=486.2 [M+H]⁺

¹H-NMR (400 MHz, DMSO-d6) δ[ppm]: 1.382 (0.96), 1.630 (6.48), 1.929 (1.08), 2.045 (3.03), 2.062 (5.55), 2.080 (3.23), 2.327 (0.54), 2.523 (5.22), 2.669 (0.57), 3.333 (16.00), 3.615 (2.89), 3.700 (1.48), 3.782 (3.39), 3.800 (5.93), 3.817 (3.36), 4.025 (0.75), 5.760 (2.99), 7.184 (2.26), 7.187 (2.28), 7.204 (2.75), 7.207 (2.72), 7.386 (2.46), 7.406 (4.58), 7.426 (2.49), 7.572 (2.61), 7.576 (2.60), 7.596 (2.19), 7.803 (5.49), 7.903 (3.24), 7.908 (5.21), 7.913 (3.09).

### Example 287

### N⁵-[(trans)-2-(3-chlorophenyl)-1-oxo-2-azaspiro[4.5]dec-8-yl]-N⁴-[2-(morpholin-4-yl)ethyl]-1H-imidazole-4,5-dicarboxamide

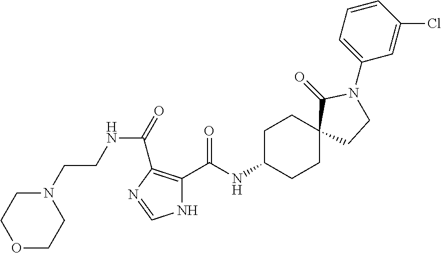 [SMILES: O=C(NCCN1CCOCC1)c1nc[nH]c1C(=O)N[C@H]1CC[C@@]2(CCN(c3cccc(Cl)c3)C2=O)CC1]

A mixture of phenyl 5-{[2-(3-chlorophenyl)-1-oxo-2-azaspiro[4.5]dec-8-yl]carbamoyl}-1H-imidazole-4-carboxylate (isomer 1) (120 mg, 243 μmol) and 2-(morpholin-4-yl)ethanamine (160 μl, 1.2 mmol, Cas No 2038-03-1) in tetrahydrofuran (3.9 ml) was stirred for 1 d at room temperature. For work-up, the reaction mixture was concentrated and the residue was purified by flash chromatography (dichloromethane/methanol-gradient, 0%->10% methanol). The obtained product was recrystallized from diethyl ether give the title compound (85.9 mg).

LC-MS (Method 1): Rₜ=0.90 min; MS (ESIpos): m/z=529.3 [M+H]⁺

¹H-NMR (400 MHz, CHLOROFORM-d) δ [ppm]: 0.000 (7.52), 1.124 (7.71), 1.141 (16.00), 1.159 (7.13), 1.425 (0.93), 1.463 (0.88), 1.601 (1.35), 1.636 (1.68), 1.852 (1.00), 1.877 (1.23), 1.885 (1.65), 2.032 (0.76), 2.049 (1.63), 2.059 (2.14), 2.067 (1.80), 2.076 (3.85), 2.086 (1.53), 2.094 (2.42), 2.108 (0.95), 2.448 (1.90), 2.460 (2.81), 2.469 (2.91), 2.491 (1.17), 2.541 (1.58), 2.556 (3.65), 2.572 (2.37), 2.591 (0.87), 3.385 (2.49), 3.403 (7.04), 3.420 (7.23), 3.438 (2.39), 3.465 (0.80), 3.479 (1.89), 3.494 (1.88), 3.509 (0.92), 3.529 (0.82), 3.543 (0.81), 3.659 (1.87), 3.674 (4.07), 3.682 (4.17), 3.687 (5.71), 3.697 (3.77), 3.705 (3.50), 3.713 (1.88), 3.723 (1.94), 3.730 (0.94), 7.035 (0.89), 7.038 (1.01), 7.040 (1.36), 7.043 (1.27), 7.056 (1.13), 7.058 (1.23), 7.061 (1.67), 7.063 (1.63), 7.201 (1.75), 7.205 (0.95), 7.222 (2.75), 7.225 (1.60), 7.242 (1.42), 7.245 (0.81), 7.498 (1.35), 7.501 (1.56), 7.504 (1.46), 7.507 (1.11), 7.519 (1.23), 7.522 (1.42), 7.525 (1.50), 7.529 (3.41), 7.538 (6.34), 7.666 (1.94), 7.672 (3.45), 7.677 (1.77), 7.961 (0.94), 11.075 (0.97), 11.094 (0.95).

### Example 288

### N⁵-[(trans)-2-(2-chloro-4-fluoro-5-methylphenyl)-1-oxo-2-azaspiro[4.5]dec-8-yl]-N⁴-methyl-1H-imidazole-4,5-dicarboxamide

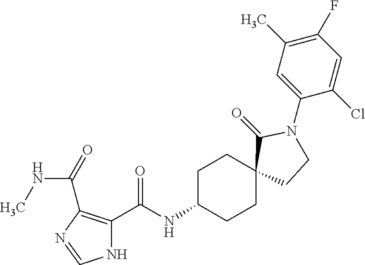 [SMILES: CNC(=O)c1nc[nH]c1C(=O)N[C@H]1CC[C@@]2(CCN(c3cc(C)c(F)cc3Cl)C2=O)CC1]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (150 mg, 263 μmol) and methanamine (660 μl, 2 M in tetrahydrofuran, 1.3 mmol) in tetrahydrofuran (4.3 ml) was stirred for 20 h at room temperature. For work-up, brine was added and the mixture was extracted with ethyl acetate. The combined organic phases were filtrated through a silicone filter, concentrated and the residue was purified by flash chromatography (25 g NH2-cartridge, dichloromethane/ethanol-gradient, 0%->21% ethanol) to give the title compound (78 mg).

LC-MS (Method 2): Rₜ=1.06 min; MS (ESIpos): m/z=462.1 [M+H]⁺

¹H-NMR (500 MHz, DMSO-d6) δ[ppm]: 1.157 (2.62), 1.172 (5.57), 1.186 (2.37), 1.656 (5.44), 1.987 (9.50), 2.116 (2.27), 2.130 (4.13), 2.143 (2.30), 2.221 (15.68), 2.225 (16.00), 2.357 (0.76), 2.361 (1.07), 2.365 (0.77), 2.514 (2.57), 2.518 (2.43), 2.522 (1.85), 2.631 (0.72), 2.634 (0.99), 2.638 (0.74), 2.813 (4.11), 3.589 (4.11), 3.603 (7.29), 3.617 (3.95), 4.004 (0.64), 4.019 (2.12), 4.033 (1.88), 4.047 (0.67), 7.367 (3.32), 7.383 (3.36), 7.488 (6.20), 7.507 (6.20), 7.810 (11.84), 13.206 (0.91).

### Example 289

### N⁵-[(trans)-2-(2-chloro-4-fluoro-5-methylphenyl)-1-oxo-2-azaspiro[4.5]dec-8-yl]-N⁴-ethyl-1H-imidazole-4,5-dicarboxamide

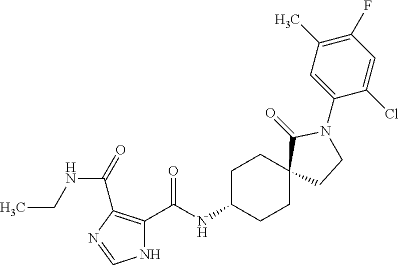 [SMILES: CCNC(=O)c1nc[nH]c1C(=O)N[C@H]1CC[C@@]2(CCN(c3cc(C)c(F)cc3Cl)C2=O)CC1]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (150 mg, 263 μmol) and ethanamine in tetrahydrofuran (660 μl, 2.0 M, 1.3 mmol) in tetrahydrofuran (4.3 ml) was stirred for 20 h at room temperature. For work-up, brine was added and the mixture was extracted with ethyl acetate. The combined organic phases were filtrated through a silicone filter, concentrated and the residue was purified by flash chromatography (25 g Snap-cartridge, dichloromethane/ethanol-gradient, 0%->18% ethanol) to give the title compound (23 mg).

LC-MS (Method 2): Rₜ=1.14 min; MS (ESIpos): m/z=476.1 [M+H]⁺

¹H-NMR (500 MHz, DMSO-d6) ([ppm]: 1.103 (5.09), 1.117 (10.67), 1.131 (8.43), 1.146 (7.77), 1.160 (3.73), 1.172 (0.70), 1.396 (1.31), 1.421 (0.94), 1.637 (5.93), 1.657 (4.17), 1.665 (4.53), 1.674 (3.58), 1.682 (3.37), 1.773 (1.55), 1.972 (1.48), 1.987 (2.16), 2.104 (1.45), 2.118 (3.08), 2.124 (2.79), 2.131 (2.25), 2.138 (4.40), 2.151 (2.28), 2.222 (15.39), 2.226 (16.00), 2.358 (0.77), 2.361 (1.07), 2.365 (0.78), 2.518 (3.46), 2.522 (2.56), 2.631 (0.73), 2.635 (1.06), 2.639 (0.78), 3.277 (0.66), 3.291 (2.71), 3.306 (4.45), 3.320 (3.66), 3.591 (2.93), 3.601 (3.87), 3.605 (4.94), 3.614 (2.23), 3.618 (2.69), 3.731 (0.68), 7.361 (1.55), 7.373 (2.66), 7.387 (2.25), 7.489 (5.06), 7.507 (4.99), 7.805 (4.89), 7.807 (4.98), 7.815 (3.39), 7.817 (3.31), 8.389 (1.31), 8.406 (1.24), 8.637 (0.85), 8.650 (1.69), 8.661 (0.83), 11.130 (1.04), 11.243 (1.94), 11.257 (1.84), 13.193 (2.08).

### Example 290

### N⁵-[(trans)-2-(2-chloro-4-fluoro-5-methylphenyl)-1-oxo-2-azaspiro[4.5]dec-8-yl]-N⁴-[2-(4-methylpiperidin-1-yl)ethyl]-1H-imidazole-4,5-dicarboxamide

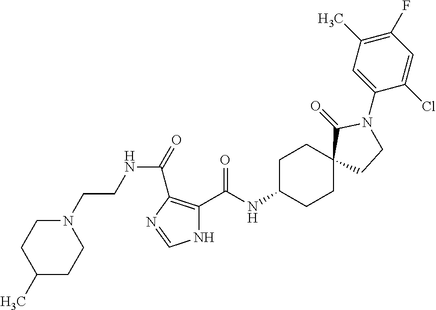 [SMILES: Cc1cc(N2CC[C@]3(CC[C@@H](NC(=O)c4[nH]cnc4C(=O)NCCN4CCC(C)CC4)CC3)C2=O)c(Cl)cc1F]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (150 mg, 263 μmol) and 2-(4-methylpiperidin-1-yl)ethanamine (187 mg, 1.31 mmol, Cas No 14156-95-7) in tetrahydrofuran (4.3 ml) was stirred for 20 h at room temperature. For work-up, brine was added and the mixture was extracted with ethyl acetate. The combined organic phases were filtrated through a silicone filter, concentrated and the residue was purified by flash chromatography (NH2-cartridge, dichloromethane/ethanol-gradient, 0%->14% ethanol) to give the title compound (122 mg).

LC-MS (Method 2): Rₜ=1.34 min; MS (ESIpos): m/z=573.4[M+H]⁺

¹H-NMR (500 MHz, DMSO-d6) δ[ppm]: 0.874 (11.38), 0.888 (11.91), 1.135 (1.31), 1.553 (2.32), 1.654 (5.21), 1.919 (2.83), 2.113 (2.45), 2.126 (4.57), 2.140 (2.49), 2.222 (15.89), 2.225 (16.00), 2.358 (0.86), 2.361 (1.26), 2.365 (0.86), 2.441 (3.88), 2.515 (3.80), 2.518 (3.23), 2.522 (2.38), 2.631 (0.80), 2.635 (1.14), 2.639 (0.82), 2.841 (2.99), 2.864 (2.85), 3.373 (1.54), 3.386 (3.86), 3.398 (3.82), 3.589 (3.92), 3.602 (7.04), 3.616 (3.84), 7.368 (3.16), 7.383 (3.14), 7.488 (6.11), 7.507 (5.86), 7.815 (13.11), 13.218 (2.23).

### Example 291

### N⁵-[(trans)-2-(2-chloro-4-fluoro-5-methylphenyl)-1-oxo-2-azaspiro[4.5]dec-8-yl]-1H-imidazole-4,5-dicarboxamide

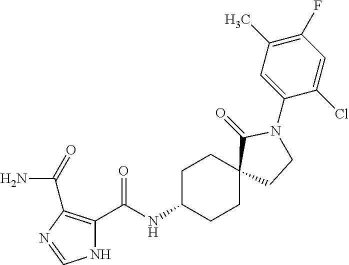 [SMILES: Cc1cc(N2CC[C@]3(CC[C@@H](NC(=O)c4[nH]cnc4C(N)=O)CC3)C2=O)c(Cl)cc1F]

phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (95.0 mg, 181 μmol) in a solution of ammonia in THF (4.5 ml, 0.40 M, 1.8 mmol) and tetrahydrofuran (4.7 ml) was stirred for 20 h at 50° C. Ammonia in THF (4.5 ml, 0.40 M, 1.8 mmol) was added and the mixture was heated to 80° C. for 3 d. For work-up, the reaction mixture was concentrated and the residue was purified by preparative HPLC (Method 8). The acetonitrile was removed under reduced pressure and the product crystalized from water/acetonitrile and was collected by filtration and dried under high vacuum to give the title compound (52 mg).

LC-MS (Method 1): Rₜ=1.02 min; MS (ESIpos): m/z=448.2 [M+H]⁺

¹H-NMR (400 MHz, DMSO-d6) δ[ppm]: 1.380 (0.74), 1.652 (5.40), 1.779 (0.86), 1.974 (0.96), 2.098 (3.88), 2.115 (7.41), 2.132 (4.03), 2.221 (15.86), 2.224 (16.00), 2.322 (0.71), 2.327 (1.00), 2.332 (0.73), 2.518 (3.07), 2.523 (1.99), 2.664 (0.69), 2.669 (1.01), 2.673 (0.73), 3.583 (4.03), 3.591 (1.25), 3.601 (7.11), 3.610 (1.22), 3.617 (3.85), 7.364 (3.17), 7.383 (3.16), 7.486 (6.36), 7.509 (6.28), 7.727 (0.68), 7.799 (2.36), 7.973 (0.83), 11.286 (0.66), 13.203 (2.51).

### Example 292

### N-[(trans)-2-(2-chloro-4-fluoro-5-methyl phenyl)-1-oxo-2-azaspiro[4.5]dec-8-yl]-4-[(3-fluoroazetidin-1-yl)carbonyl]-1H-imidazole-5-carboxamide

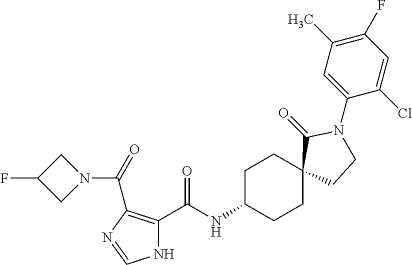 [SMILES: Cc1cc(N2CC[C@]3(CC[C@@H](NC(=O)c4[nH]cnc4C(=O)N4CC(F)C4)CC3)C2=O)c(Cl)cc1F]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (95.0 mg, 181 μmol), 3-fluoroazetidine hydrochloride (40.4 mg, 362 μmol, Cas No 617718-46-4) and triethylamine (76 μl) in tetrahydrofuran (4.7 ml) was stirred for 20 h at 50° C. For work-up, the reaction mixture was concentrated and the residue was purified by preparative HPLC (Method 8). The product crystalized from water/acetonitrile and was collected by filtration and dried under high vacuum to give the title compound (62 mg).

LC-MS (Method 1): Rₜ=1.14 min; MS (ESIpos): m/z=606.2 [M+H]⁺

¹H-NMR (400 MHz, DMSO-d6) δ[ppm]: 1.348 (1.37), 1.376 (1.97), 1.648 (6.16), 1.657 (7.01), 1.672 (5.18), 1.954 (2.26), 1.975 (2.04), 1.984 (2.17), 2.074 (0.61), 2.107 (3.70), 2.124 (6.97), 2.142 (3.92), 2.221 (16.00), 2.226 (15.33), 2.323 (0.94), 2.327 (1.30), 2.331 (0.94), 2.523 (2.69), 2.665 (0.99), 2.669 (1.30), 2.674 (0.92), 3.583 (3.94), 3.601 (7.33), 3.618 (3.83), 3.705 (0.96), 3.716 (1.17), 3.733 (1.21), 4.073 (0.94), 4.103 (1.12), 4.136 (1.01), 4.165 (1.12), 4.376 (0.72), 4.387 (0.87), 4.422 (0.85), 4.441 (0.81), 4.461 (0.69), 4.585 (0.81), 4.616 (0.96), 4.645 (0.83), 4.676 (0.94), 5.351 (0.72), 5.358 (0.99), 5.365 (1.19), 5.373 (0.94), 5.502 (1.03), 5.509 (1.17), 5.517 (0.96), 7.367 (4.21), 7.386 (4.17), 7.486 (5.78), 7.509 (5.74), 7.829 (14.41), 11.069 (2.53), 11.087 (2.42), 13.295 (2.24).

### Example 293

### N-[(trans)-2-(2-chloro-4-fluoro-5-methyl phenyl)-1-oxo-2-azaspiro[4.5]dec-8-yl]-4-(2-oxa-6-azaspiro[3.3]hept-6-ylcarbonyl)-1H-imidazole-5-carboxamide

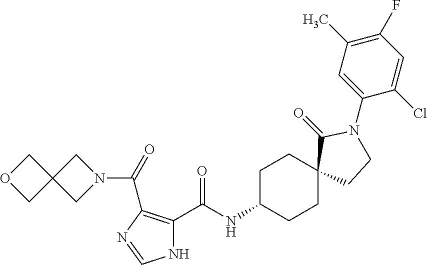 [SMILES: Cc1cc(N2CC[C@]3(CC[C@@H](NC(=O)c4[nH]cnc4C(=O)N4CC5(COC5)C4)CC3)C2=O)c(Cl)cc1F]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (95.0 mg, 181 μmol), 2-oxa-6-azaspiro[3.3]heptane ethanedioate (2:1) (52.2 mg, 181 μmol, Cas No 1045709-32-7) and triethylamine (76 μl) in tetrahydrofuran (1.8 ml) was stirred for 20 h at 50° C. 2-oxa-6-azaspiro[3.3]heptane ethanedioate (2:1) (26.1 mg, 90.5 μmol) was added and the mixture was stirred for 3 d at 50° C. For work-up, the reaction mixture was concentrated and the residue was purified by preparative HPLC (Method 8). The product crystalized from water/acetonitrile and was collected by filtration and dried under high vacuum to give the title compound (52 mg).

LC-MS (Method 1): Rₜ=1.06 min; MS (ESIpos): m/z=530.3 [M+H]⁺

¹H-NMR (400 MHz, DMSO-d6) δ[ppm]: 1.333 (1.17), 1.343 (1.15), 1.363 (1.94), 1.383 (1.37), 1.393 (1.33), 1.643 (5.30), 1.652 (6.14), 1.665 (4.66), 1.673 (4.57), 1.934 (2.05), 1.943 (2.17), 1.954 (1.50), 1.966 (2.05), 1.976 (1.83), 2.075 (0.89), 2.106 (3.38), 2.123 (6.56), 2.140 (3.63), 2.221 (14.30), 2.225 (14.34), 2.327 (1.00), 2.332 (0.73), 2.518 (3.65), 2.523 (2.39), 2.669 (1.04), 3.583 (3.67), 3.601 (6.50), 3.618 (3.54), 3.701 (1.04), 3.720 (1.02), 4.248 (9.88), 4.674 (2.90), 4.691 (12.15), 4.699 (12.05), 4.717 (2.85), 4.768 (9.50), 7.366 (3.82), 7.385 (3.65), 7.386 (3.73), 7.487 (5.81), 7.510 (5.75), 7.811 (16.00), 11.165 (2.96), 11.183 (2.78), 13.240 (3.12).

### Example 294

### N-[(trans)-2-(2-chloro-4-fluoro-5-methyl phenyl)-1-oxo-2-azaspiro[4.5]dec-8-yl]-4-[(4-methylpiperazin-1-yl)carbonyl]-1H-imidazole-5-carboxamide

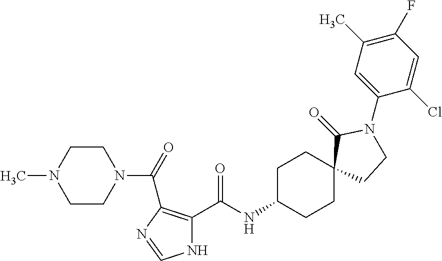 [SMILES: Cc1cc(N2CC[C@]3(CC[C@@H](NC(=O)c4[nH]cnc4C(=O)N4CCN(C)CC4)CC3)C2=O)c(Cl)cc1F]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (95.0 mg, 181 μmol), 1-methylpiperazine (100 μl, 900 μmol) in tetrahydrofuran (2.9 ml) was stirred for 21 h at 50° C. For work-up, the reaction mixture was concentrated and the residue was purified by preparative HPLC (Method 8) The product crystalized from water/acetonitrile and was collected by filtration and dried under high vacuum to give the title compound (59 mg).

LC-MS (Method 1): Rₜ=0.83 min; MS (ESIpos): m/z=532.5 [M+H]⁺

¹H-NMR (400 MHz, DMSO-d6) δ[ppm]: 1.352 (1.55), 1.650 (5.56), 1.955 (1.88), 2.097 (3.04), 2.114 (5.23), 2.130 (3.28), 2.197 (13.63), 2.224 (16.00), 2.361 (5.75), 2.668 (1.01), 3.580 (3.71), 3.597 (6.59), 3.614 (4.23), 3.661 (3.12), 3.965 (2.44), 7.363 (3.01), 7.383 (3.04), 7.485 (3.96), 7.509 (3.97), 7.802 (3.43), 8.146 (2.27), 9.913 (1.44), 9.930 (1.39), 13.212 (1.14).

### Example 295

### N⁵-[(trans)-2-(2-chloro-4-fluoro-5-methylphenyl)-1-oxo-2-azaspiro[4.5]dec-8-yl]-N⁴-cyclopropyl-1H-imidazole-4,5-dicarboxamide

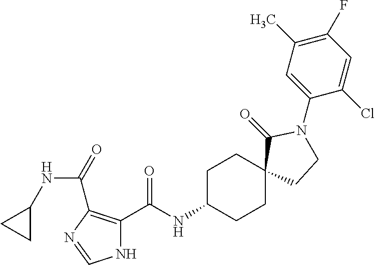 [SMILES: Cc1cc(N2CC[C@]3(CC[C@@H](NC(=O)c4[nH]cnc4C(=O)NC4CC4)CC3)C2=O)c(Cl)cc1F]

A mixture of phenyl 5-{[(2-(2-chloro-4-fluoro-5-methylphenyl)-1-oxo-2-azaspiro[4.5]dec-8-yl]carbamoyl}-1H-imidazole-4-carboxylate (isomer 1) (95.0 mg, 181 μmol) cyclopropanamine (63 μl, 900 μmol, Cas No 765-30-0) in tetrahydrofuran (2.9 ml) was stirred for 21 h at 50° C. For work-up, the reaction mixture was concentrated and the residue was purified by preparative HPLC (Method 8). The product crystalized from water/acetonitrile and was collected by filtration and dried under high vacuum to give the title compound (67 mg).

LC-MS (Method 1): Rₜ=1.17 min; MS (ESIpos): m/z=488.2 [M+H]⁺

¹H-NMR (400 MHz, DMSO-d6) δ [ppm]: 0.504 (1.56), 0.683 (3.41), 0.752 (1.48), 1.414 (0.86), 1.636 (5.41), 1.757 (1.28), 1.975 (1.08), 2.074 (1.20), 2.131 (2.35), 2.222 (16.00), 2.227 (15.55), 2.327 (0.79), 2.523 (2.17), 2.669 (0.74), 2.881 (0.76), 3.586 (2.70), 3.603 (4.83), 3.620 (2.67), 7.365 (2.10), 7.384 (2.07), 7.486 (4.97), 7.509 (4.86), 7.797 (1.80), 7.817 (1.70), 8.418 (0.63), 8.574 (0.80), 11.183 (0.74), 11.247 (0.86), 13.224 (1.16).

### Example 296

### N-[(trans)-2-(2-chloro-4-fluoro-5-methyl phenyl)-1-oxo-2-azaspiro[4.5]dec-8-yl]-4-{[3-(difluoromethoxy)azetidin-1-yl]carbonyl}-1H-imidazole-5-carboxamide

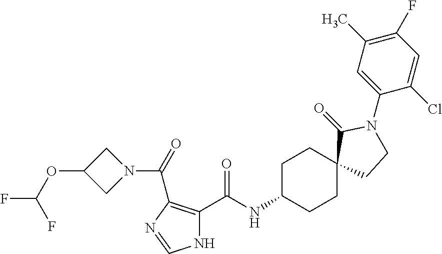 [SMILES: Cc1cc(N2CC[C@]3(CC[C@@H](NC(=O)c4[nH]cnc4C(=O)N4CC(OC(F)F)C4)CC3)C2=O)c(Cl)cc1F]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (85.0 mg, 162 μmol), 3-(difluoromethoxy)azetidine (39.9 mg, 324 μmol) and triethylamine (68 μl) in tetrahydrofuran (1.6 ml) was stirred for 20 h at 50° C. 3-(difluoromethoxy)azetidine 19.95 mg, 162 μmol) was added and the mixture was stirred for 3 d at 50° C. For work-up, the reaction mixture was concentrated and the residue was purified by preparative HPLC (Method 8). The product crystalized from water/acetonitrile and was collected by filtration and dried under high vacuum to give the title compound (44 mg).

LC-MS (Method 1): Rₜ=1.22 min; MS (ESIpos): m/z=554.4 [M+H]⁺

¹H-NMR (400 MHz, DMSO-d6) δ[ppm]: 1.347 (1.19), 1.375 (1.72), 1.397 (1.29), 1.647 (5.36), 1.657 (6.12), 1.670 (4.49), 1.679 (4.22), 1.951 (1.95), 1.960 (1.81), 1.973 (1.76), 1.983 (1.83), 2.107 (3.44), 2.124 (6.33), 2.142 (3.62), 2.221 (14.39), 2.225 (14.19), 2.327 (1.03), 2.518 (2.90), 2.523 (2.10), 2.669 (1.03), 2.673 (0.72), 3.583 (3.71), 3.601 (6.68), 3.618 (3.55), 3.713 (1.03), 4.008 (1.30), 4.014 (1.38), 4.036 (1.56), 4.045 (1.41), 4.405 (1.25), 4.409 (1.30), 4.421 (1.48), 4.426 (1.52), 4.433 (1.25), 4.437 (1.30), 4.454 (1.18), 4.522 (1.14), 4.532 (1.29), 4.554 (1.41), 4.561 (1.43), 4.914 (1.09), 4.927 (1.52), 4.931 (1.54), 4.956 (1.36), 5.002 (1.36), 5.010 (1.45), 5.019 (1.94), 5.028 (1.19), 5.035 (0.92), 6.614 (2.82), 6.801 (5.90), 6.987 (2.52), 7.366 (3.67), 7.367 (3.76), 7.385 (3.76), 7.387 (3.69), 7.487 (5.88), 7.510 (5.85), 7.825 (16.00), 11.067 (2.84), 11.085 (2.66), 13.297 (2.95).

### Example 297

### N⁵-[(trans)-2-(2-chloro-4-fluoro-5-methylphenyl)-1-oxo-2-azaspiro[4.5]dec-8-yl]-N⁴-(2,2,2-trifluoroethyl)-1H-imidazole-4,5-dicarboxamide

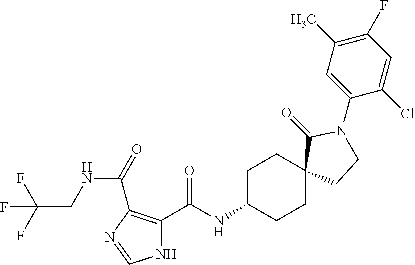 [SMILES: Cc1cc(N2CC[C@]3(CC[C@@H](NC(=O)c4[nH]cnc4C(=O)NCC(F)(F)F)CC3)C2=O)c(Cl)cc1F]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (150 mg, 263 μmol) and 2,2,2-trifluoroethanamine hydrochloride (178 mg, 1.31 mmol, Cas No 373-88-6) in pyridine (10 ml, 120 mmol) was heated in a microwave reactor for 15 min at 130° C. followed by 75 min at 170° C. For work-up, the reaction mixture was concentrated and the residue was purified by flash chromatography (dichloromethane/ethanol-gradient 0%->13% ethanol) followed by flash chromatography (dichloromethane/ethanol-gradient 0%->20% ethanol) to give the title compound (33 mg).

LC-MS (Method 2): Rₜ=1.15 min; MS (ESIpos): m/z=530.1 [M+H]⁺

¹H-NMR (500 MHz, DMSO-d6) δ[ppm]: 1.232 (1.37), 1.650 (6.39), 2.123 (4.41), 2.221 (15.93), 2.225 (16.00), 2.361 (1.51), 2.518 (4.41), 2.522 (3.27), 2.634 (1.35), 3.587 (4.09), 3.601 (7.41), 3.615 (4.02), 7.364 (2.81), 7.380 (2.86), 7.487 (5.60), 7.506 (5.57), 7.911 (1.72).

### Example 298

### N-[(trans)-2-(2-chloro-4,5-difluorophenyl)-1-oxo-2-azaspiro[4.5]dec-8-yl]imidazo[1,2-b]pyridazine-3-carboxamide

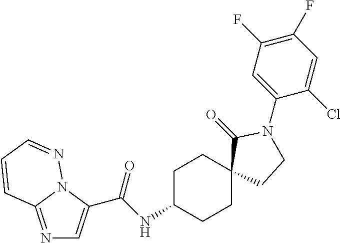 [SMILES: O=C(N[C@H]1CC[C@@]2(CCN(c3cc(F)c(F)cc3Cl)C2=O)CC1)c1cnc2cccnn12]

Benzotriazol-1-yl-oxytripyrrolidinophosphonium hexafluorophosphate (267 mg, 512 μmol) and N,N-diisopropylethylamine (325 μl, 1.9 mmol) were added to a mixture of imidazo[1,2-b]pyridazine-3-carboxylic acid (80.0 mg, 466 μmol, Cas No 1308384-58-8) and 8-amino-2-(2-chloro-4,5-difluorophenyl)-2-azaspiro[4.5]decan-1-one (isomer 1) (161 mg, 512 μmol) in N,N-dimethylformamide (4.7 ml) and the mixture was stirred at room temperature for 24 h. For work-up, the reaction mixture was concentrated and the residue was purified by flash chromatography (dichloromethane/methanol-gradient 0%->10% methanol) The crude product was recrystallized from methanol to give the title compound (138.6 mg).

LC-MS (Method 2): Rₜ=1.06 min; MS (ESIpos): m/z=460.1[M+H]⁺

¹H-NMR (400 MHz, CHLOROFORM-d) δ [ppm]: 0.000 (2.80), 1.422 (0.81), 1.431 (0.79), 1.451 (0.79), 1.460 (0.93), 1.566 (16.00), 1.668 (0.91), 1.703 (1.20), 1.900 (0.69), 1.909 (0.80), 1.934 (1.16), 1.944 (1.28), 2.140 (2.01), 2.148 (1.20), 2.157 (3.97), 2.175 (2.27), 2.181 (1.10), 2.191 (0.89), 3.605 (1.99), 3.622 (3.27), 3.640 (1.90), 5.233 (0.72), 7.064 (1.06), 7.084 (1.17), 7.090 (1.14), 7.109 (1.10), 7.160 (1.42), 7.171 (1.46), 7.183 (1.52), 7.226 (1.15), 7.245 (1.18), 7.250 (1.20), 7.270 (1.07), 8.061 (1.53), 8.065 (1.55), 8.084 (1.44), 8.088 (1.49), 8.342 (0.71), 8.361 (0.70), 8.438 (1.30), 8.443 (1.37), 8.450 (1.37), 8.454 (1.33), 8.463 (5.15).

### Example 299

### N-[(trans)-2-(2-chloro-4,5-difluorophenyl)-1-oxo-2-azaspiro[4.5]dec-8-yl]-5,7-dimethylpyrazolo[1,5-a]pyrimidine-3-carboxamide

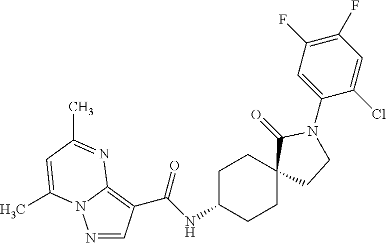 [SMILES: Cc1cc(C)n2ncc(C(=O)N[C@H]3CC[C@@]4(CCN(c5cc(F)c(F)cc5Cl)C4=O)CC3)c2n1]

Benzotriazol-1-yl-oxytripyrrolidinophosphonium hexafluorophosphate (228 mg, 437 μmol) and N,N-diisopropylethylamine (280 μl, 1.6 mmol) were added to a mixture of 5,7-dimethylpyrazolo[1,5-a]pyrimidine-3-carboxylic acid (80.0 mg, 398 μmol Cas No 90349-23-8) 8-amino-2-(2-chloro-4,5-difluorophenyl)-2-azaspiro[4.5]decan-1-one (isomer 1) (138 mg, 437 μmol) in N,N-dimethylformamide (4.0 ml) and the mixture was stirred at room temperature for 24 h. For work-up, the reaction mixture was concentrated and the residue was purified by flash chromatography (dichloromethane/methanol-gradient 0%->10% methanol) The crude product was recrystallized from methanol to give the title compound (119.2 mg).

LC-MS (Method 2): Rₜ=1.22 min; MS (ESIpos): m/z=488.2 [M+H]⁺

¹H-NMR (400 MHz, CHLOROFORM-d) δ [ppm]: 0.000 (1.11), 1.552 (16.00), 1.691 (0.90), 1.926 (0.89), 1.935 (0.94), 2.139 (1.43), 2.156 (2.85), 2.173 (1.73), 2.588 (11.50), 2.724 (6.82), 2.726 (6.76), 3.599 (1.48), 3.617 (2.44), 3.634 (1.42), 6.641 (1.97), 6.644 (1.97), 7.066 (0.78), 7.085 (0.87), 7.091 (0.85), 7.110 (0.81), 7.221 (0.85), 7.240 (0.89), 7.245 (0.89), 7.265 (0.80), 8.554 (3.62).

### Example 300

### N-[(trans)-2-(2-chloro-4-fluoro-5-methyl phenyl)-1-oxo-2-azaspiro[5.5]undec-9-yl]pyrazolo[1,5-a]pyrimidine-3-carboxamide

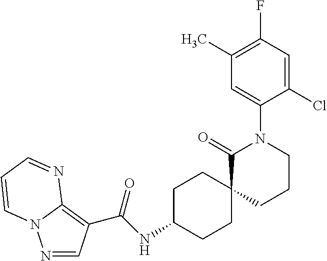 [SMILES: Cc1cc(N2CCC[C@]3(CC[C@@H](NC(=O)c4cnn5cccnc45)CC3)C2=O)c(Cl)cc1F]

Benzotriazol-1-yl-oxytripyrrolidinophosphonium hexafluorophosphate (96 mg, 185 μmol) and N,N-diisopropylethylamine (120 μl) were added to a mixture of pyrazolo[1,5-a]pyrimidine-3-carboxylic acid (28 mg, 168 μmol, Cas No 25940-35-6) and 9-amino-2-(2-chloro-4-fluoro-5-methylphenyl)-2-azaspiro[5.5]undecan-1-one (isomer 1) (60.0 mg, 185 μmol) in N,N-dimethylformamide (1.7 ml) and the mixture was stirred at room temperature for 5 h. For work-up, water (50 ml) and brine were added and the precipitate formed was collected by filtration, washed with water and dried to give the title compound (72 mg).

LC-MS (Method 1): Rₜ=1.17 min; MS (ESIpos): m/z=470.2 [M+H]⁺

¹H-NMR (400 MHz, DMSO-d6) δ[ppm]: 1.517 (2.15), 1.661 (1.56), 1.695 (1.85), 1.755 (1.34), 1.788 (1.95), 1.858 (4.79), 1.887 (4.47), 1.912 (6.75), 1.943 (6.66), 1.978 (2.36), 2.221 (16.00), 2.326 (0.96), 2.668 (0.99), 3.507 (1.88), 3.520 (1.62), 3.808 (1.28), 7.257 (2.67), 7.268 (2.91), 7.275 (2.99), 7.285 (2.82), 7.320 (3.86), 7.339 (3.81), 7.450 (4.41), 7.474 (4.40), 7.833 (3.15), 7.852 (3.11), 8.567 (9.22), 8.812 (3.21), 8.816 (3.71), 8.823 (3.68), 8.827 (3.51), 9.305 (3.24), 9.309 (3.39), 9.323 (3.36), 9.326 (3.41).

### Example 301

### N-[(trans)-2-(2-chloro-4-fluoro-5-methyl phenyl)-1-oxo-2-azaspiro[5.5]undec-9-yl]imidazo[1,2-b]pyridazine-3-carboxamide

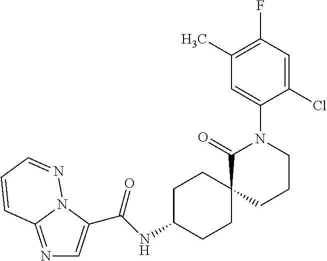 [SMILES: Cc1cc(N2CCC[C@]3(CC[C@@H](NC(=O)c4cnc5cccnn45)CC3)C2=O)c(Cl)cc1F]

Benzotriazol-1-yl-oxytripyrrolidinophosphonium hexafluorophosphate (96 mg, 185 μmol) and N,N-diisopropylethylamine (120 μl) were added to a mixture of imidazo[1,2-b]pyridazine-3-carboxylic acid (28 mg, 168 μmol, Cas No 1308384-58-8) 9-amino-2-(2-chloro-4-fluoro-5-methylphenyl)-2-azaspiro[5.5]undecan-1-one (isomer 1) (60 mg, 185 μmol) in N,N-dimethylformamide (1.7 ml) and the mixture was stirred at room temperature for 5 h. For work-up, water (50 ml) was added and the mixture was stirred overnight. The precipitated formed was collected by filtration, washed with water and dried at 50° C. to give the title compound (72 mg).

LC-MS (Method 1): Rₜ=1.12 min; MS (ESIpos): m/z=470.3 [M+H]⁺

¹H-NMR (400 MHz, DMSO-d6) δ[ppm]: 1.575 (1.94), 1.673 (1.35), 1.700 (1.60), 1.764 (1.14), 1.797 (1.68), 1.867 (4.26), 1.892 (4.06), 1.919 (5.95), 1.935 (4.04), 1.949 (6.21), 1.986 (2.00), 2.218 (15.88), 2.222 (16.00), 3.355 (1.48), 3.371 (1.68), 3.513 (1.74), 3.525 (1.43), 3.859 (1.17), 7.321 (4.12), 7.341 (4.11), 7.449 (5.67), 7.452 (6.48), 7.460 (4.38), 7.472 (5.65), 7.476 (6.42), 7.484 (4.46), 8.293 (14.74), 8.335 (4.27), 8.339 (4.57), 8.358 (4.37), 8.362 (4.30), 8.497 (3.36), 8.517 (3.27), 8.778 (3.94), 8.782 (4.08), 8.790 (4.02), 8.793 (3.69).

### Example 302

### N-[(trans)-2-(2-chloro-4-fluoro-5-methyl phenyl)-1-oxo-2-azaspiro[5.5]undec-9-yl]-5,7-dimethylpyrazolo[1,5-a]pyrimidine-3-carboxamide

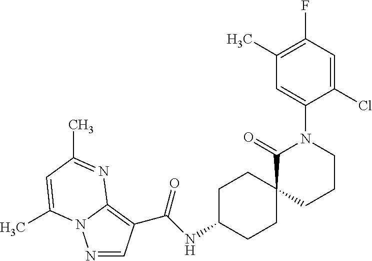 [SMILES: Cc1cc(C)n2ncc(C(=O)N[C@H]3CC[C@@]4(CCCN(c5cc(C)c(F)cc5Cl)C4=O)CC3)c2n1]

Benzotriazol-1-yl-oxytripyrrolidinophosphonium hexafluorophosphate (96.1 mg, 185 μmol) and N,N-diisopropylethylamine (120 μl) were added to a mixture of 5,7-dimethylpyrazolo[1,5-a]pyrimidine-3-carboxylic acid (33.1 mg, 168 μmol, Cas No 90349-23-8) and 9-amino-2-(2-chloro-4-fluoro-5-methylphenyl)-2-azaspiro[5.5]undecan-1-one (isomer 1) (60.0 mg, 185 μmol) in N,N-dimethylformamide (1.7 ml) and the mixture was stirred at room temperature for 5 h. For work-up, water (50 ml) and the mixture was stirred overnight. The precipitated formed was collected by filtration, washed with water and dried at 50° C. to give the title compound (86 mg).

LC-MS (Method 1): Rₜ=1.29 min; MS (ESIpos): m/z=498.3 [M+H]⁺

¹H-NMR (400 MHz, DMSO-d6) δ[ppm]: 1.883 (1.78), 1.909 (3.02), 1.933 (3.21), 1.945 (3.58), 1.961 (2.00), 2.218 (7.61), 2.222 (7.37), 2.634 (16.00), 2.736 (11.09), 7.110 (3.65), 7.307 (1.96), 7.327 (1.96), 7.422 (2.62), 7.446 (2.58), 8.011 (1.58), 8.031 (1.52), 8.301 (1.90), 8.474 (6.34).

### Example 303

### N-[(trans)-2-(2-chloro-4-fluoro-5-methyl phenyl)-1-oxo-2-azaspiro[5.5]undec-9-yl]-4-[(3-fluoroazetidin-1-yl)carbonyl]-1H-imidazole-5-carboxamide

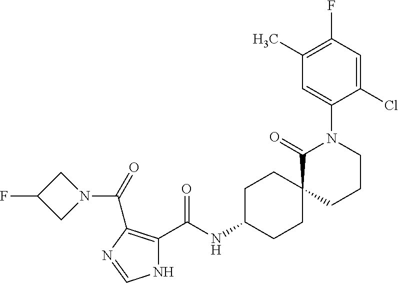 [SMILES: Cc1cc(N2CCC[C@]3(CC[C@@H](NC(=O)c4[nH]cnc4C(=O)N4CC(F)C4)CC3)C2=O)c(Cl)cc1F]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (150 mg, 278 μmol), 3-fluoroazetidine hydrochloride (62.1 mg, 557 μmol, Cas No 617718-46-4) and triethylamine (120 μl) in tetrahydrofuran (4.5 ml) was stirred for 2 at room temperature. For work-up, the reaction mixture was concentrated and the residue was purified by flash chromatography (25 g Snap Cartridge, dichloromethane/methanol-gradient, 0%->7% methanol). The obtained product was dissolved in DMSO and water was added to precipitate the product. The precipitate was collected by filtration and dried to give the title compound (65 mg).

LC-MS (Method 2): Rₜ=1.17 min; MS (ESIpos): m/z=518.5 [M+H]⁺

¹H-NMR (400 MHz, DMSO-d6) δ[ppm]: 1.232 (1.45), 1.395 (1.88), 1.419 (1.55), 1.643 (1.23), 1.678 (1.35), 1.735 (1.02), 1.769 (1.59), 1.838 (3.04), 1.864 (4.58), 1.904 (10.16), 1.951 (1.33), 2.075 (1.00), 2.213 (15.98), 2.217 (16.00), 2.322 (0.80), 2.326 (1.08), 2.332 (0.82), 2.522 (3.27), 2.664 (0.80), 2.668 (1.08), 2.673 (0.82), 3.358 (2.10), 3.502 (1.53), 3.531 (1.19), 3.702 (1.12), 4.071 (0.86), 4.102 (1.08), 4.135 (0.90), 4.165 (1.04), 4.373 (0.72), 4.387 (0.82), 4.423 (0.90), 4.443 (0.80), 4.458 (0.67), 4.582 (0.92), 4.614 (1.12), 4.619 (0.94), 4.644 (0.94), 4.675 (1.06), 4.884 (0.72), 4.894 (0.88), 4.915 (0.67), 4.930 (0.80), 4.941 (0.78), 4.950 (0.84), 4.972 (0.69), 5.350 (0.78), 5.357 (1.06), 5.364 (1.27), 5.372 (1.00), 5.501 (1.02), 5.509 (1.23), 5.516 (1.02), 5.523 (0.74), 7.317 (3.88), 7.336 (3.84), 7.448 (5.84), 7.471 (5.74), 7.823 (9.24), 7.826 (9.17), 11.069 (3.45), 11.088 (3.29), 13.297 (3.70).

### Example 304

### N⁵-[(trans)-2-(2-chloro-4-fluoro-5-methylphenyl)-1-oxo-2-azaspiro[5.5]undec-9-yl]-N⁴-methyl-1H-imidazole-4,5-dicarboxamide

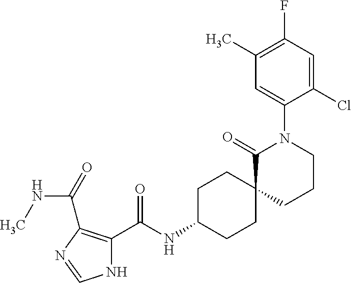 [SMILES: CNC(=O)c1nc[nH]c1C(=O)N[C@H]1CC[C@@]2(CCCN(c3cc(C)c(F)cc3Cl)C2=O)CC1]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (150 mg, 278 μmol) and methanamine in tetrahydrofuran (2.0 ml, 2.0 M, 4.0 mmol) was stirred for 2 d at room temperature. For work-up, the reaction mixture was concentrated and the residue was purified by flash chromatography (25 g Snap Cartridge, dichloromethane/methanol-gradient, 0%->7% methanol). The residue was dissolved in DMSO and water was added to precipitate product. The precipitate was collected by filtration and dried to give the title compound (40 mg).

LC-MS (Method 1): Rₜ=1.16 min; MS (ESIpos): m/z=476.1 [M+H]⁺

¹H-NMR (400 MHz, DMSO-d6) δ[ppm]: 1.415 (1.08), 1.653 (2.37), 1.769 (1.52), 1.914 (7.31), 2.214 (16.00), 2.218 (15.81), 2.322 (0.83), 2.327 (1.16), 2.332 (0.85), 2.523 (2.99), 2.539 (0.89), 2.664 (0.89), 2.669 (1.21), 2.673 (0.89), 2.796 (4.16), 2.807 (4.95), 3.359 (1.72), 3.504 (1.37), 7.317 (2.53), 7.336 (2.47), 7.449 (5.78), 7.472 (5.74), 7.804 (4.47), 8.616 (0.93), 11.262 (1.04), 13.196 (1.37).

### Example 305

### N⁵-[(trans)-2-(2-chloro-4-fluoro-5-methylphenyl)-1-oxo-2-azaspiro[5.5]undec-9-yl]-N⁴-ethyl-1H-imidazole-4,5-dicarboxamide

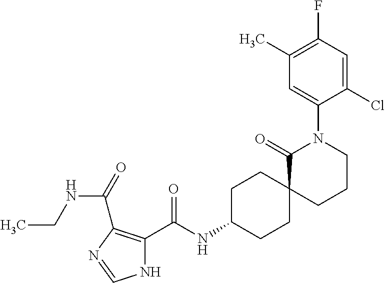 [SMILES: CCNC(=O)c1nc[nH]c1C(=O)N[C@H]1CC[C@@]2(CCCN(c3cc(C)c(F)cc3Cl)C2=O)CC1]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (150 mg, 278 μmol) and ethanamine in tetrahydrofuran (2.0 ml, 2.0 M, 4.0 mmol) was stirred for 2 d at room temperature. For work-up, the reaction mixture was concentrated and the residue was purified by flash chromatography (dichloromethane/methanol-gradient, 0%->9% methanol) to give the title compound (67 mg).

LC-MS (Method 1): Rₜ=1.23 min; MS (ESIpos): m/z=490.3 [M+H]⁺

¹H-NMR (400 MHz, DMSO-d6) b [ppm]: 1.108 (3.27), 1.126 (6.16), 1.143 (3.32), 1.651 (2.24), 1.677 (2.07), 1.737 (1.09), 1.769 (1.60), 1.914 (6.59), 2.214 (16.00), 2.218 (15.73), 2.322 (0.71), 2.327 (0.98), 2.332 (0.71), 2.518 (3.23), 2.523 (1.99), 2.664 (0.71), 2.669 (1.01), 2.673 (0.72), 3.272 (1.16), 3.290 (3.76), 3.304 (4.61), 3.308 (4.40), 3.323 (5.14), 3.359 (1.55), 3.371 (1.06), 3.501 (1.36), 7.316 (3.17), 7.335 (3.17), 7.449 (6.42), 7.472 (6.30), 7.806 (7.11), 13.200 (1.73).

### Example 306

### N⁵-[(trans)-2-(2-chloro-4-fluoro-5-methylphenyl)-1-oxo-2-azaspiro[5.5]undec-9-yl]-N⁴-(2-methoxyethyl)-1H-imidazole-4,5-dicarboxamide

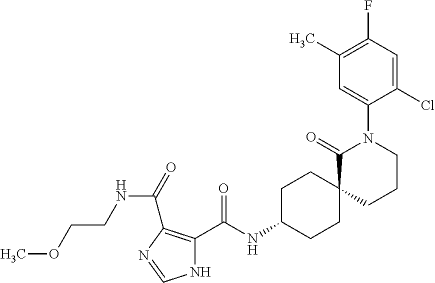 [SMILES: COCCNC(=O)c1nc[nH]c1C(=O)N[C@H]1CC[C@@]2(CCCN(c3cc(C)c(F)cc3Cl)C2=O)CC1]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (150 mg, 278 μmol), 2-methoxyethanamine (41.8 mg, 557 μmol, Cas No 109-85-3) and triethylamine (120 μl) in tetrahydrofuran (4.5 ml) was stirred for 2 d at room temperature. For work-up, the reaction mixture was concentrated and the residue was purified by flash chromatography (25 g Snap Cartridge, dichloromethane/methanol-gradient, 0%->10% methanol) to give the title compound (62 mg).

LC-MS (Method 1): Rₜ=1.19 min; MS (ESIpos): m/z=520.3 [M+H]⁺

¹H-NMR (400 MHz, DMSO-d6) δ[ppm]: 1.651 (1.32), 1.846 (0.97), 1.912 (3.57), 2.214 (8.45), 2.218 (8.25), 2.523 (1.14), 3.271 (16.00), 3.455 (4.84), 3.465 (5.70), 7.319 (1.13), 7.338 (1.10), 7.449 (3.30), 7.472 (3.26), 7.815 (2.17), 13.236 (1.41).

### Example 307

### N⁴-[(trans)-2-(2-chloro-4-fluorophenyl)-3-hydroxy-1-oxo-2-azaspiro[4.5]dec-8-yl]-N⁵-methyl-1H-imidazole-4,5-dicarboxamide (mixture of isomers at C₃)

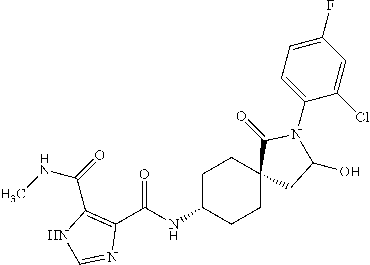

To a mixture of N⁴-[(trans)-2-(2-chloro-4-fluorophenyl)-1-oxo-2-azaspiro[4.5]dec-8-yl]-N⁵-methyl-1H-imidazole-4,5-dicarboxamide (Example 140) (300 mg, 670 μmol) in tert-butanol (6.0 ml) and 1 M aqueous sulphuric acid (6.0 ml) was added cerium(IV) sulfate (667 mg, 2.0 mmol) and the mixture stirred overnight at 40° C. Additional water (20 ml) was added, and the mixture extracted with dichloromethane (3×40 ml). The organic phases were combined, dried by passage through a water repellent filter and the solvent removed under reduced pressure, affording the title compound, after purification by preparative HPLC (8 mg).

LC-MS (Method 1): Rₜ=0.86 min; MS (ESIpos): m/z=464.4 [M+H]⁺

¹H-NMR (600 MHz, DMSO-d6) δ[ppm]: 0.000 (6.90), 1.116 (0.41), 1.234 (0.66), 1.362 (0.74), 1.368 (0.81), 1.387 (1.15), 1.408 (0.86), 1.497 (0.75), 1.517 (0.90), 1.534 (0.56), 1.569 (1.22), 1.586 (2.10), 1.622 (2.56), 1.642 (1.94), 1.672 (1.00), 1.694 (1.26), 1.715 (1.75), 1.735 (1.30), 1.768 (1.23), 1.783 (1.92), 1.797 (2.06), 1.909 (1.62), 1.913 (1.76), 1.931 (1.79), 1.936 (1.80), 1.984 (1.22), 1.990 (1.17), 2.075 (1.76), 2.087 (3.33), 2.098 (1.87), 2.384 (0.50), 2.387 (0.71), 2.390 (0.56), 2.415 (0.43), 2.426 (0.46), 2.437 (0.47), 2.449 (0.85), 2.460 (0.97), 2.471 (1.03), 2.482 (1.30), 2.518 (3.13), 2.521 (2.68), 2.524 (2.06), 2.543 (4.48), 2.612 (0.51), 2.615 (0.70), 2.619 (0.53), 2.805 (4.87), 2.813 (5.15), 2.842 (2.41), 2.849 (2.23), 3.569 (0.44), 3.595 (2.21), 3.606 (3.69), 3.618 (2.05), 3.716 (0.59), 3.728 (0.60), 5.353 (1.53), 5.758 (16.00), 6.328 (0.89), 7.285 (0.77), 7.291 (1.61), 7.296 (1.37), 7.300 (1.64), 7.305 (3.34), 7.310 (2.57), 7.314 (1.46), 7.319 (2.14), 7.324 (1.60), 7.376 (1.29), 7.386 (1.61), 7.400 (0.86), 7.449 (1.47), 7.460 (1.56), 7.464 (1.35), 7.474 (1.26), 7.569 (1.62), 7.573 (1.84), 7.578 (2.33), 7.583 (3.69), 7.588 (2.22), 7.593 (2.27), 7.598 (2.19), 7.807 (3.72), 7.817 (1.65), 8.389 (0.55), 8.403 (0.55), 8.621 (1.01), 8.629 (1.03), 11.047 (0.48), 11.053 (0.48), 11.255 (1.27), 11.267 (1.22), 13.198 (0.63).

### Example 308

### N⁵-[(trans)-2-(2-chloro-4-fluoro-5-methylphenyl)-1-oxo-2-azaspiro[5.5]undec-9-yl]-N⁴-cyclopropyl-1H-imidazole-4,5-dicarboxamide

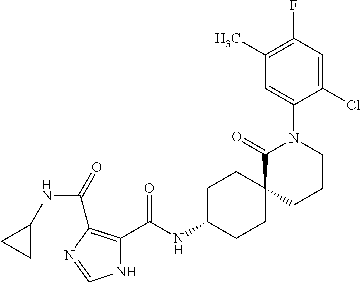 [SMILES: Cc1cc(N2CCC[C@]3(CC[C@@H](NC(=O)c4[nH]cnc4C(=O)NC4CC4)CC3)C2=O)c(Cl)cc1F]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (125 mg, 139 μmol), cyclopropanamine (15.9 mg, 278 μmol, Cas No 765-30-0) in tetrahydrofuran (2.3 ml) was stirred for 17 h at room temperature. For work-up, the reaction mixture was concentrated and the residue was purified by flash chromatography (25 g Snap Cartridge, dichloromethane/methanol-gradient, 0%->22% methanol). The residue was dissolved in DMSO and water was added the product was purified by preparative HPLC to give the title compound (27 mg).

LC-MS (Method 2): Rₜ=1.18 min; MS (ESIpos): m/z=502.2 [M+H]⁺

¹H-NMR (400 MHz, DMSO-d6) δ[ppm]: 0.499 (1.57), 0.684 (3.14), 0.744 (1.55), 0.759 (1.36), 1.433 (0.74), 1.641 (2.21), 1.758 (1.17), 1.897 (4.62), 1.908 (4.74), 1.921 (6.14), 1.961 (1.24), 2.214 (16.00), 2.218 (16.00), 2.323 (1.00), 2.327 (1.43), 2.332 (1.00), 2.518 (4.00), 2.523 (2.81), 2.665 (1.02), 2.669 (1.45), 2.673 (1.02), 2.837 (0.67), 3.359 (1.48), 3.509 (1.19), 7.316 (2.00), 7.335 (1.98), 7.449 (6.19), 7.473 (6.05), 7.791 (1.60), 7.818 (1.64), 8.439 (0.67), 8.459 (0.67), 8.571 (0.76), 11.181 (0.69), 11.201 (0.67), 11.260 (0.86), 13.221 (1.14).

### Example 309

### N⁵-[(trans)-2-(2-chloro-4-fluoro-5-methylphenyl)-1-oxo-2-azaspiro[5.5]undec-9-yl]-1H-imidazole-4,5-dicarboxamide

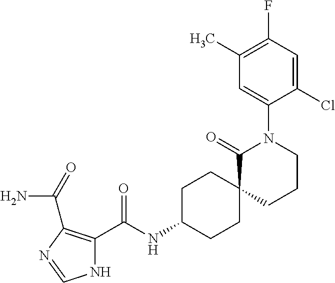 [SMILES: Cc1cc(N2CCC[C@]3(CC[C@@H](NC(=O)c4[nH]cnc4C(N)=O)CC3)C2=O)c(Cl)cc1F]

phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (85.0 mg, 158 μmol) in a solution of ammonia in THF (3.9 ml, 0.40 M, 1.6 mmol) and tetrahydrofuran (5.0 ml) was stirred for 20 h at 50° C. Ammonia in THF (3.9 ml, 0.40 M, 1.6 mmol) was added and the mixture was heated to 100° C. for 90 min in a microwave reactor, followed 8 0° C. for 3 d. For work-up, the reaction mixture was concentrated and the residue was purified by preparative HPLC (Method 8). The product crystalized from water/acetonitrile and was collected by filtration and dried under high vacuum to give the title compound (27 mg).

LC-MS (Method 1): Rₜ=1.07 min; MS (ESIpos): m/z=462.2 [M+H]⁺

¹H-NMR (400 MHz, DMSO-d6) δ[ppm]: 1.378 (0.75), 1.387 (0.72), 1.397 (0.86), 1.405 (1.07), 1.424 (0.86), 1.432 (0.79), 1.640 (3.14), 1.660 (3.72), 1.725 (1.22), 1.760 (1.73), 1.793 (0.72), 1.807 (0.67), 1.826 (0.79), 1.849 (1.84), 1.855 (1.81), 1.896 (8.65), 1.902 (7.82), 1.919 (5.13), 1.952 (1.51), 1.972 (0.79), 2.213 (16.00), 2.218 (15.65), 2.323 (0.75), 2.327 (1.02), 2.331 (0.74), 2.518 (3.39), 2.523 (2.20), 2.665 (0.75), 2.669 (1.01), 2.674 (0.72), 3.357 (1.86), 3.504 (1.39), 3.532 (0.92), 3.722 (0.69), 3.741 (0.86), 7.310 (2.06), 7.318 (2.45), 7.328 (2.16), 7.338 (2.33), 7.446 (5.42), 7.469 (5.35), 7.700 (1.31), 7.709 (1.29), 7.774 (1.86), 7.784 (6.17), 7.813 (4.28), 7.815 (4.19), 7.962 (1.71), 8.388 (1.61), 8.410 (1.56), 10.442 (1.29), 10.450 (1.17), 11.280 (1.91), 11.298 (1.81), 13.196 (2.58).

### Example 310

### N⁵-[(trans)-2-(2-chloro-4-fluoro-5-methylphenyl)-1-oxo-2-azaspiro[5.5]undec-9-yl]-N⁴-(propan-2-yl)-1H-imidazole-4,5-dicarboxamide

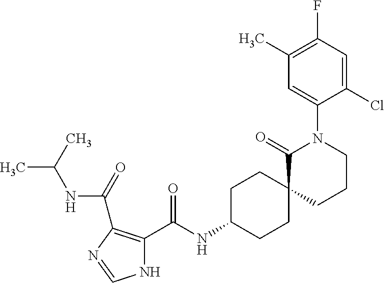 [SMILES: Cc1cc(N2CCC[C@]3(CC[C@@H](NC(=O)c4[nH]cnc4C(=O)NC(C)C)CC3)C2=O)c(Cl)cc1F]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (85.0 mg, 158 μmol), propan-2-amine (68 μl, 790 μmol, Cas No 75-31-0) and triethylamine (66 μl) in tetrahydrofuran (1.5 ml) was stirred for 22 h at 50° C. For work-up, the reaction mixture was concentrated and the residue was purified by preparative HPLC (Method 8). The product crystalized from water/acetonitrile and was collected by filtration and dried under high vacuum to give the title compound (32 mg).

LC-MS (Method 1): Rₜ=1.28 min; MS (ESIpos): m/z=504.3 [M+H]⁺

¹H-NMR (400 MHz, DMSO-d6) b [ppm]: 1.171 (15.96), 1.187 (16.00), 1.233 (0.74), 1.417 (0.74), 1.651 (3.02), 1.764 (1.35), 1.843 (1.61), 1.912 (5.78), 2.074 (1.72), 2.214 (15.04), 2.219 (14.67), 2.323 (0.98), 2.327 (1.33), 2.331 (0.93), 2.518 (4.24), 2.523 (2.76), 2.665 (0.98), 2.669 (1.33), 2.674 (0.93), 3.347 (1.37), 3.360 (1.54), 3.510 (1.22), 3.994 (0.74), 4.010 (0.74), 7.312 (1.89), 7.331 (1.89), 7.448 (5.43), 7.471 (5.35), 7.804 (5.13), 8.274 (0.76), 8.297 (0.76), 8.408 (1.04), 8.430 (1.02), 11.097 (0.98), 11.115 (0.96), 11.206 (0.85), 11.225 (0.78), 13.199 (2.02).

### Example 311

### N-[(trans)-2-(2-chloro-4-fluoro-5-methylphenyl)-1-oxo-2-azaspiro[5.5]undec-9-yl]-4-(2-oxa-6-azaspiro[3.3]hept-6-ylcarbonyl)-1H-imidazole-5-carboxamide

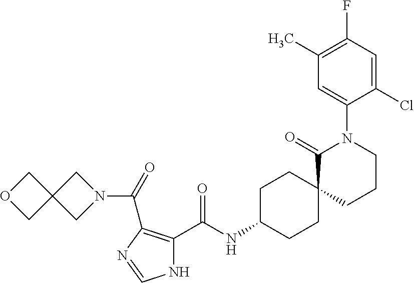 [SMILES: Cc1cc(N2CCC[C@]3(CC[C@@H](NC(=O)c4[nH]cnc4C(=O)N4CC5(COC5)C4)CC3)C2=O)c(Cl)cc1F]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (85.0 mg, 158 μmol), 2-oxa-6-azaspiro[3.3]heptane ethanedioate (2:1) (90.95 mg, 315 μmol, Cas No 1045709-32-7) and triethylamine (66 μl) in tetrahydrofuran (1.5 ml) was stirred for 20 h at 50° C. 2-oxa-6-azaspiro[3.3]heptane ethanedioate (2:1) (45.5 mg, 157.5 μmol) was added and the mixture was stirred for 5 h at 50° C. For work-up, the reaction mixture was concentrated and the residue was purified by preparative HPLC (Method 8). The product crystalized from water/acetonitrile and was collected by filtration and dried under high vacuum to give the title compound (40 mg).

LC-MS (Method 1): Rₜ=1.11 min; MS (ESIpos): m/z=544.3 [M+H]⁺

¹H-NMR (400 MHz, DMSO-d6) δ[ppm]: 1.352 (1.36), 1.362 (1.23), 1.381 (1.92), 1.411 (1.54), 1.643 (1.27), 1.675 (1.40), 1.735 (1.05), 1.769 (1.54), 1.833 (3.05), 1.840 (2.83), 1.859 (3.84), 1.869 (3.03), 1.886 (4.73), 1.904 (9.32), 1.912 (6.49), 1.925 (4.01), 1.947 (1.36), 2.213 (15.68), 2.218 (16.00), 2.323 (0.85), 2.327 (1.17), 2.331 (0.85), 2.518 (4.39), 2.523 (2.89), 2.665 (0.87), 2.669 (1.21), 2.673 (0.87), 3.344 (1.33), 3.359 (1.70), 3.490 (0.83), 3.503 (1.56), 3.513 (1.19), 3.532 (1.13), 3.681 (0.91), 3.690 (1.09), 3.708 (1.11), 3.719 (0.87), 4.248 (11.21), 4.676 (2.99), 4.693 (14.12), 4.700 (13.94), 4.717 (2.99), 4.766 (11.25), 7.316 (4.17), 7.336 (4.17), 7.447 (5.97), 7.471 (5.85), 7.804 (9.22), 7.806 (8.92), 11.161 (3.50), 11.179 (3.30), 13.234 (3.70).

### Example 312

### N⁵-[(trans)-2-(2-chloro-4-fluoro-5-methylphenyl)-1-oxo-2-azaspiro[5.5]undec-9-yl]-N⁴-[2-(4-methylpiperidin-1-yl)ethyl]-1H-imidazole-4,5-dicarboxamide

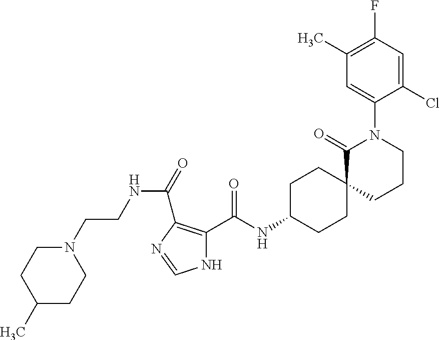 [SMILES: Cc1cc(N2CCC[C@]3(CC[C@@H](NC(=O)c4[nH]cnc4C(=O)NCCN4CCC(C)CC4)CC3)C2=O)c(Cl)cc1F]

A mixture of phenyl 5-{[2-(2-chloro-4-fluoro-5-methylphenyl)-1-oxo-2-azaspiro[5.5]undec-9-yl]carbamoyl}-1H-imidazole-4-carboxylate (isomer 1) (150 mg, 278 μmol), 2-(4-methylpiperidin-1-yl)ethanamine (79.2 mg, 557 μmol, Cas No 14156-95-7) and triethylamine (120 μl) in tetrahydrofuran (4.5 ml) was stirred for 2 d at room temperature. For work-up, the reaction mixture was concentrated and the residue was purified by flash chromatography (dichloromethane/methanol-gradient, 0%->10% methanol) to give the title compound (57 mg).

LC-MS (Method 1): Rₜ=0.96 min; MS (ESIpos): m/z=590.6 [M+H]⁺

¹H-NMR (400 MHz, DMSO-d6) δ[ppm]: 0.860 (3.31), 0.875 (9.04), 0.892 (6.35), 1.103 (1.36), 1.134 (1.99), 1.164 (1.40), 1.410 (1.17), 1.556 (2.60), 1.587 (1.62), 1.663 (3.10), 1.767 (1.59), 1.912 (9.37), 2.213 (16.00), 2.217 (15.99), 2.327 (0.85), 2.449 (3.16), 2.518 (2.94), 2.523 (1.93), 2.669 (0.88), 2.846 (2.37), 3.367 (2.69), 3.383 (4.45), 3.398 (4.29), 3.415 (1.59), 3.509 (1.30), 7.319 (2.41), 7.339 (2.31), 7.447 (6.00), 7.471 (5.91), 7.811 (11.01), 8.374 (0.82), 8.395 (0.83), 8.437 (1.39), 11.147 (0.79), 11.190 (1.54), 11.208 (1.45), 13.221 (1.81).

### Example 313

### N-[(trans)-2-(2-chloro-4,5-difluorophenyl)-1-oxo-2-azaspiro[5.5]undec-9-yl]pyrazolo[1,5-a]pyrimidine-3-carboxamide

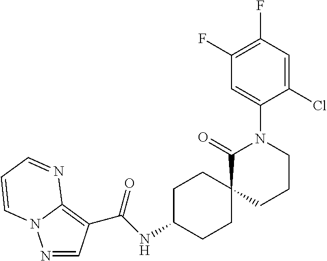 [SMILES: O=C(N[C@H]1CC[C@@]2(CCCN(c3cc(F)c(F)cc3Cl)C2=O)CC1)c1cnn2cccnc12]

Benzotriazol-1-yl-oxytripyrrolidinophosphonium hexafluorophosphate (95.0 mg, 182 μmol) and N,N-diisopropylethylamine (120 μl, 660 μmol) were added to a mixture of pyrazolo[1,5-a]pyrimidine-3-carboxylic acid (27.9 mg, 166 μmol, Cas No 25940-35-6) and 9-amino-2-(2-chloro-4,5-difluorophenyl)-2-azaspiro[5.5]undecan-1-one (isomer 1) (60.0 mg, 182 μmol) in N,N-dimethylformamide (1.7 ml) and the mixture was stirred at room temperature for 23 h. For work-up, water (50 ml) and brine were added and the precipitate formed was collected by filtration, washed with water and dried. The crude product was recrystallized from methanol/water to give the title compound (23 mg).

LC-MS (Method 2): Rₜ=1.13 min; MS (ESIpos): m/z=474.5 [M+H]⁺

¹H-NMR (400 MHz, DMSO-d6) δ[ppm]: 1.487 (1.40), 1.520 (1.98), 1.549 (1.62), 1.660 (1.33), 1.695 (1.57), 1.772 (1.06), 1.811 (2.15), 1.844 (3.72), 1.857 (3.12), 1.869 (3.70), 1.878 (3.46), 1.916 (5.78), 1.928 (4.93), 1.952 (3.24), 1.960 (2.73), 1.969 (2.18), 1.993 (1.93), 2.323 (1.06), 2.327 (1.47), 2.332 (1.06), 2.518 (5.73), 2.523 (3.72), 2.665 (1.04), 2.669 (1.47), 2.673 (1.06), 3.354 (1.62), 3.368 (1.64), 3.551 (1.52), 3.562 (1.31), 3.579 (1.18), 3.800 (0.97), 3.810 (1.18), 3.820 (1.04), 3.829 (1.16), 7.259 (4.23), 7.269 (3.96), 7.276 (3.99), 7.287 (4.33), 7.663 (2.78), 7.684 (3.12), 7.691 (2.97), 7.711 (2.80), 7.830 (3.77), 7.836 (4.01), 7.850 (4.13), 7.856 (6.36), 7.877 (3.05), 8.568 (16.00), 8.812 (4.64), 8.817 (5.32), 8.823 (4.95), 8.827 (4.57), 9.307 (5.05), 9.311 (4.98), 9.325 (4.76), 9.329 (4.93).

### Example 314

### N-[(trans)-2-(2-chloro-4,5-difluorophenyl)-1-oxo-2-azaspiro[5.5]undec-9-yl]imidazo[1,2-b]pyridazine-3-carboxamide

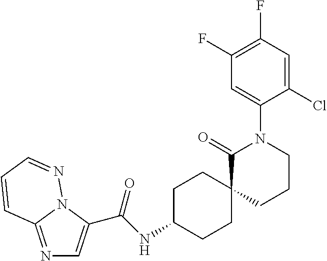 [SMILES: O=C(N[C@H]1CC[C@@]2(CCCN(c3cc(F)c(F)cc3Cl)C2=O)CC1)c1cnc2cccnn12]

Benzotriazol-1-yl-oxytripyrrolidinophosphonium hexafluorophosphate (95.0 mg, 182 μmol) and N,N-diisopropylethylamine (120 μl, 660 μmol) were added to a mixture of imidazo[1,2-b]pyridazine-3-carboxylic acid (27.9 mg, 166 μmol, Cas No 1308384-58-8) and 9-amino-2-(2-chloro-4,5-difluorophenyl)-2-azaspiro[5.5]undecan-1-one (isomer 1) (60.0 mg, 182 μmol) in N,N-dimethylformamide (1.7 ml) and the mixture was stirred at room temperature for 5 h. For work-up, water (50 ml) and brine were added and the precipitate formed was collected by filtration, washed with water and dried. The crude product was recrystallized from methanol/water to give the title compound (32 mg).

LC-MS (Method 1): Rₜ=1.09 min; MS (ESIpos): m/z=474.2 [M+H]⁺

¹H-NMR (400 MHz, DMSO-d6) b [ppm]: 1.546 (1.38), 1.579 (2.07), 1.608 (1.66), 1.671 (1.29), 1.705 (1.54), 1.818 (1.91), 1.850 (3.51), 1.858 (2.81), 1.877 (4.24), 1.912 (4.78), 1.923 (5.99), 1.934 (4.79), 1.957 (2.97), 1.967 (2.69), 1.979 (1.87), 1.991 (1.44), 2.002 (1.88), 2.327 (0.90), 2.523 (2.37), 2.669 (0.90), 3.358 (1.51), 3.372 (1.60), 3.556 (1.51), 3.566 (1.31), 3.584 (1.22), 3.851 (0.97), 3.861 (1.19), 3.871 (1.04), 3.880 (1.19), 7.450 (4.62), 7.461 (4.31), 7.473 (4.29), 7.484 (4.63), 7.664 (2.74), 7.684 (3.06), 7.692 (2.93), 7.712 (2.79), 7.831 (2.78), 7.851 (3.07), 7.858 (3.19), 7.878 (2.90), 8.293 (16.00), 8.336 (4.97), 8.340 (5.10), 8.359 (4.59), 8.364 (4.53), 8.500 (3.46), 8.520 (3.37), 8.777 (4.12), 8.782 (4.28), 8.789 (4.22), 8.793 (3.84).

### Example 315

### N-[(trans)-2-(2-chloro-4,5-difluorophenyl)-1-oxo-2-azaspiro[5.5]undec-9-yl]-5,7-dimethylpyrazolo[1,5-a]pyrimidine-3-carboxamide

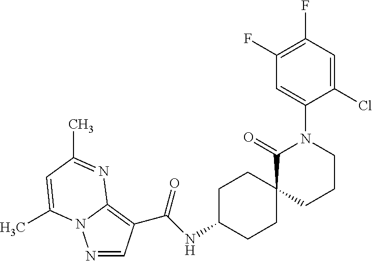 [SMILES: Cc1cc(C)n2ncc(C(=O)N[C@H]3CC[C@@]4(CCCN(c5cc(F)c(F)cc5Cl)C4=O)CC3)c2n1]

Benzotriazol-1-yl-oxytripyrrolidinophosphonium hexafluorophosphate (95.0 mg, 182 μmol) and N,N-diisopropylethylamine (120 μl, 660 μmol) were added to a mixture of 5,7-dimethylpyrazolo[1,5-a]pyrimidine-3-carboxylic acid (32.7 mg, 166 μmol, Cas No 90349-23-8) and 9-amino-2-(2-chloro-4,5-difluorophenyl)-2-azaspiro[5.5]undecan-1-one (isomer 1) (60.0 mg, 182 μmol) in N,N-dimethylformamide (1.7 ml) and the mixture was stirred at room temperature for 5 h. For work-up, water (50 ml) and brine were added and the precipitate formed was collected by filtration, washed with water and dried. The crude product was recrystallized from methanol/water to give the title compound (28 mg).

LC-MS (Method 1): Rₜ=1.26 min; MS (ESIpos): m/z=502.2 [M+H]⁺

¹H-NMR (400 MHz, DMSO-d6) δ[ppm]: 1.500 (0.92), 1.854 (1.22), 1.882 (2.26), 1.913 (2.79), 1.927 (3.33), 1.957 (1.76), 1.988 (0.91), 2.630 (16.00), 2.730 (10.43), 2.732 (10.40), 7.118 (3.46), 7.121 (3.51), 7.661 (1.13), 7.682 (1.32), 7.689 (1.25), 7.709 (1.17), 7.826 (1.17), 7.846 (1.32), 7.853 (1.33), 7.872 (1.22), 8.016 (1.56), 8.036 (1.51), 8.482 (6.67).

### Example 316

### N-[(trans)-2-(2-chloro-4,5-difluorophenyl)-1-oxo-2-azaspiro[5.5]undec-9-yl]-4-[(3-fluoroazetidin-1-yl)carbonyl]-1H-imidazole-5-carboxamide

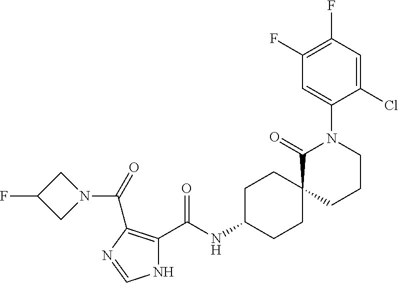 [SMILES: O=C(N[C@H]1CC[C@@]2(CCCN(c3cc(F)c(F)cc3Cl)C2=O)CC1)c1[nH]cnc1C(=O)N1CC(F)C1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (125 mg, 138 μmol), 3-fluoroazetidine hydrochloride (30.8 mg, 276 μmol, Cas No 617718-46-4) and triethylamine (58 μl) in tetrahydrofuran (2.2 ml) was stirred for 2 d at room temperature. For work-up, the reaction mixture was concentrated and the residue was purified by flash chromatography (25 g Snap Cartridge, dichloromethane/methanol-gradient, 0%->20% methanol) to give the title compound (39 mg).

LC-MS (Method 1): Rₜ=1.16 min; MS (ESIpos): m/z=524.3 [M+H]⁺

¹H-NMR (400 MHz, DMSO-d6) δ [ppm]: 1.367 (1.22), 1.394 (1.86), 1.420 (1.29), 1.643 (1.11), 1.677 (1.18), 1.787 (1.58), 1.824 (2.42), 1.851 (3.81), 1.898 (6.00), 1.915 (5.13), 1.925 (4.99), 1.958 (1.34), 2.323 (0.99), 2.327 (1.39), 2.332 (0.99), 2.518 (4.12), 2.523 (2.94), 2.665 (0.99), 2.669 (1.41), 2.673 (0.99), 3.358 (1.58), 3.548 (1.27), 3.559 (1.20), 3.576 (0.96), 3.706 (1.04), 3.724 (1.04), 4.071 (0.87), 4.102 (1.06), 4.135 (0.89), 4.164 (0.99), 4.583 (0.85), 4.615 (1.01), 4.619 (0.85), 4.644 (0.87), 4.674 (0.96), 4.894 (0.80), 4.899 (0.78), 5.350 (0.78), 5.357 (1.04), 5.365 (1.20), 5.372 (0.94), 5.502 (0.96), 5.509 (1.15), 5.517 (0.96), 7.660 (2.07), 7.681 (2.35), 7.688 (2.26), 7.708 (2.07), 7.825 (16.00), 7.846 (2.89), 7.852 (3.04), 7.872 (2.85), 11.072 (3.22), 11.090 (3.04), 13.299 (3.22).

### Example 317

### N⁵-[(trans)-2-(2-chloro-4,5-difluorophenyl)-1-oxo-2-azaspiro[5.5]undec-9-yl]-N⁴-methyl-1H-imidazole-4,5-dicarboxamide

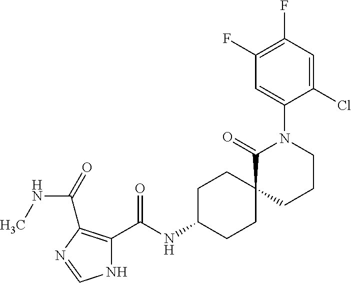 [SMILES: CNC(=O)c1nc[nH]c1C(=O)N[C@H]1CC[C@@]2(CCCN(c3cc(F)c(F)cc3Cl)C2=O)CC1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (125 mg, 138 μmol) and methanamine in THF (990 μl, 2.0 M, 2.0 mmol) was stirred for 18 h at room temperature. For work-up, the reaction mixture was concentrated and the residue was purified by flash chromatography (25 g Snap Cartridge, dichloromethane/methanol-gradient, 0%->20% methanol) to give the title compound (55 mg).

LC-MS (Method 1): Rₜ=1.11 min; MS (ESIpos): m/z=480.2 [M+H]⁺

¹H-NMR (400 MHz, DMSO-d6) δ[ppm]: 1.052 (0.74), 1.232 (1.59), 1.416 (2.72), 1.653 (6.30), 1.678 (5.73), 1.791 (5.73), 1.904 (16.00), 2.318 (1.13), 2.322 (2.38), 2.327 (3.35), 2.332 (2.44), 2.337 (1.02), 2.518 (12.88), 2.523 (8.62), 2.660 (1.19), 2.665 (2.55), 2.669 (3.46), 2.673 (2.50), 2.679 (1.08), 2.808 (13.16), 3.361 (4.31), 3.546 (3.63), 3.575 (2.78), 3.729 (1.93), 5.760 (4.48), 7.660 (4.03), 7.681 (5.11), 7.687 (4.88), 7.708 (3.97), 7.806 (14.35), 7.820 (2.44), 7.827 (7.94), 7.847 (8.17), 7.853 (8.51), 7.873 (7.94), 8.454 (1.02), 8.628 (2.21), 11.055 (1.08), 11.265 (2.44), 11.281 (2.33), 13.197 (3.63).

### Example 318

### N⁵-[(trans)-2-(2-chloro-4,5-difluorophenyl)-1-oxo-2-azaspiro[5.5]undec-9-yl]-N⁴-ethyl-1H-imidazole-4,5-dicarboxamide

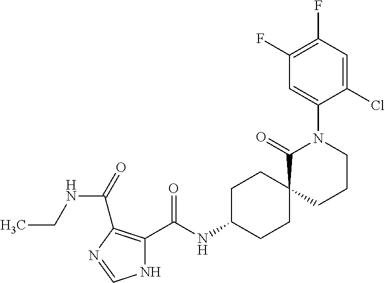 [SMILES: CCNC(=O)c1nc[nH]c1C(=O)N[C@H]1CC[C@@]2(CCCN(c3cc(F)c(F)cc3Cl)C2=O)CC1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (125 mg, 138 μmol) and ethanamine in THF (990 μl, 2.0 M, 2.0 mmol) was stirred for 19 h at room temperature. For work-up, the reaction mixture was concentrated and the residue was purified by flash chromatography (25 g Snap Cartridge, dichloromethane/ethanol-gradient, 0%->20% ethanol) to give after recrystallization from methanol the title compound (8 mg).

LC-MS (Method 1): Rₜ=1.16 min; MS (ESIpos): m/z=494.3 [M+H]⁺

¹H-NMR (400 MHz, DMSO-d6) δ[ppm]: 1.109 (7.81), 1.126 (14.63), 1.144 (8.00), 1.232 (1.23), 1.417 (1.85), 1.651 (5.59), 1.677 (5.21), 1.794 (5.07), 1.906 (13.40), 2.323 (2.22), 2.327 (3.03), 2.331 (2.22), 2.523 (7.01), 2.665 (2.27), 2.669 (3.08), 2.673 (2.27), 3.272 (2.65), 3.290 (8.38), 3.305 (10.89), 3.360 (3.64), 3.546 (3.17), 3.574 (2.41), 3.739 (1.37), 5.759 (0.62), 7.657 (3.98), 7.678 (4.83), 7.685 (4.59), 7.705 (3.98), 7.805 (16.00), 7.825 (6.20), 7.845 (6.63), 7.852 (6.72), 7.871 (6.20), 8.432 (0.85), 8.643 (1.47), 11.141 (1.04), 11.251 (1.51), 13.198 (4.21).

### Example 319

### 6-bromo-N-[(trans)-2-(2-chloro-4-fluorophenyl)-1-oxo-2-azaspiro[4.5]dec-8-yl]-1-isopropyl-1H-indazole-3-carboxamide

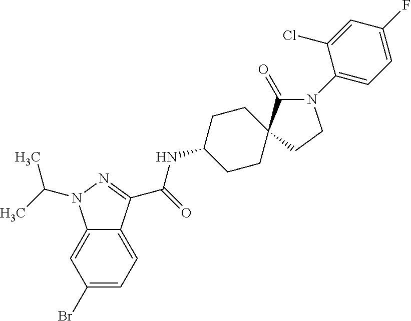 [SMILES: CC(C)n1nc(C(=O)N[C@H]2CC[C@@]3(CCN(c4ccc(F)cc4Cl)C3=O)CC2)c2ccc(Br)cc21]

Was prepared in analogy to example 247 using (trans)-8-amino-2-(2-chloro-4-fluorophenyl)-2-azaspiro[4.5]decan-1-one and 6-bromo-1-isopropyl-1H-indazole-3-carboxylic acid as staring materials.

LC-MS (method see example 247): Rₜ=1.41 min; MS (ESIpos): m/z=561 [M+H]⁺

### Example 320

### N⁵-[(trans)-2-(2-chloro-4,5-difluorophenyl)-1-oxo-2-azaspiro[5.5]undec-9-yl]-1H-imidazole-4,5-dicarboxamide

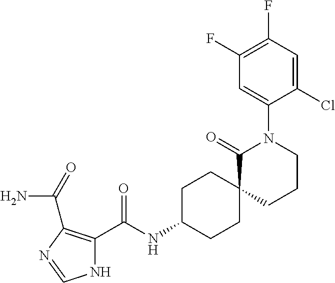 [SMILES: NC(=O)c1nc[nH]c1C(=O)N[C@H]1CC[C@@]2(CCCN(c3cc(F)c(F)cc3Cl)C2=O)CC1]

phenyl 5-{[2-(2-chloro-4,5-difluorophenyl)-1-oxo-2-azaspiro[5.5]undec-9-yl]carbamoyl}-1H-imidazole-4-carboxylate (isomer 1) (85.0 mg, 157 μmol) in a solution of ammonia in THF (3.9 ml, 0.40 M, 1.6 mmol) and tetrahydrofuran (5.0 ml) was stirred for 3 d at 80° C. For work-up, the reaction mixture was concentrated and the residue was purified by preparative HPLC (Method 8). The product crystalized from water/acetonitrile and was collected by filtration and dried under high vacuum to give the title compound (37 mg).

LC-MS (Method 1): Rₜ=1.02 min; MS (ESIpos): m/z=466.3 [M+H]⁺

¹H-NMR (400 MHz, DMSO-d6) δ[ppm]: 1.232 (0.87), 1.379 (1.75), 1.411 (2.55), 1.436 (1.90), 1.462 (0.80), 1.640 (6.45), 1.660 (8.09), 1.753 (1.90), 1.779 (5.47), 1.838 (2.95), 1.862 (6.16), 1.899 (16.00), 1.925 (9.51), 1.957 (2.66), 1.966 (2.70), 1.986 (1.64), 2.074 (5.61), 2.318 (0.69), 2.323 (1.57), 2.327 (2.19), 2.331 (1.60), 2.337 (0.69), 2.518 (8.09), 2.523 (5.25), 2.660 (0.80), 2.665 (1.64), 2.669 (2.22), 2.674 (1.57), 2.678 (0.73), 3.344 (3.97), 3.359 (3.61), 3.547 (2.99), 3.567 (2.30), 3.747 (2.04), 3.759 (1.97), 7.650 (2.81), 7.660 (3.57), 7.671 (3.50), 7.681 (5.03), 7.688 (4.12), 7.699 (5.47), 7.709 (6.23), 7.774 (4.23), 7.783 (13.89), 7.787 (11.92), 7.813 (9.55), 7.816 (9.51), 7.823 (5.76), 7.844 (6.09), 7.850 (6.05), 7.870 (5.61), 7.963 (3.90), 8.389 (3.57), 8.411 (3.46), 10.440 (2.88), 10.448 (2.62), 11.284 (4.23), 11.302 (4.08), 13.197 (5.83).

### Example 321

### N⁵-[(trans)-2-(2-chloro-4,5-difluorophenyl)-1-oxo-2-azaspiro[5.5]undec-9-yl]-N⁴-(propan-2-yl)-1H-imidazole-4,5-dicarboxamide

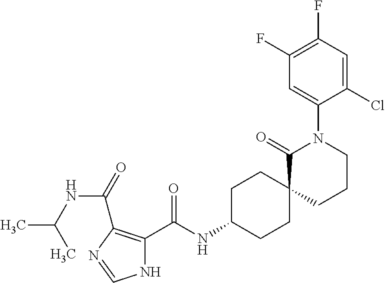 [SMILES: CC(C)NC(=O)c1nc[nH]c1C(=O)N[C@H]1CC[C@@]2(CCCN(c3cc(F)c(F)cc3Cl)C2=O)CC1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (85.0 mg, 157 μmol), propan-2-amine (67 μl, 780 μmol, Cas No 75-31-0) and triethylamine (65 μl) in tetrahydrofuran (1.5 ml) was stirred for 3 d at 50° C. For work-up, the reaction mixture was concentrated and the residue was purified by preparative HPLC (Method 8). The product crystalized from water/acetonitrile and was collected by filtration and dried under high vacuum to give the title compound (32 mg).

LC-MS (Method 1): Rₜ=1.22 min; MS (ESIpos): m/z=508.2 [M+H]⁺

¹H-NMR (400 MHz, DMSO-d6) b [ppm]: 1.172 (16.00), 1.188 (15.87), 1.650 (2.31), 1.791 (1.43), 1.911 (4.02), 2.074 (1.32), 2.323 (0.69), 2.327 (0.96), 2.332 (0.66), 2.518 (3.00), 2.523 (1.98), 2.665 (0.69), 2.669 (0.96), 2.674 (0.66), 3.348 (1.04), 3.360 (1.09), 3.543 (0.90), 7.656 (0.93), 7.681 (1.27), 7.704 (0.95), 7.804 (5.72), 7.825 (2.10), 7.845 (2.25), 7.851 (2.28), 7.871 (2.17), 13.199 (1.62).

### Example 322

### N-[(trans)-2-(2-chloro-4,5-difluorophenyl)-1-oxo-2-azaspiro[5.5]undec-9-yl]-4-{[3-(difluoromethoxy)azetidin-1-yl]carbonyl}-1H-imidazole-5-carboxamide

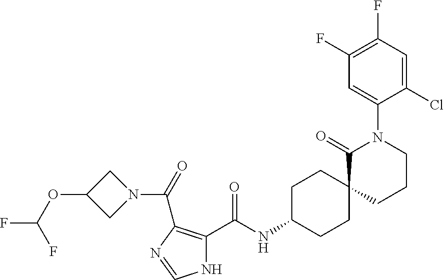 [SMILES: O=C(N[C@H]1CC[C@@]2(CCCN(c3cc(F)c(F)cc3Cl)C2=O)CC1)c1[nH]cnc1C(=O)N1CC(OC(F)F)C1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (85.0 mg, 157 μmol), 3-(difluoromethoxy)azetidine (38.5 mg, 313 μmol) and triethylamine (65 μl) in tetrahydrofuran (1.5 ml) was stirred for 3 d at 50° C. For work-up, the reaction mixture was concentrated and the residue was purified by preparative HPLC (Method 8). The product crystalized from water/acetonitrile and was collected by filtration and dried under high vacuum to give the title compound (35 mg).

LC-MS (Method 1): Rₜ=1.22 min; MS (ESIpos): m/z=572.6 [M+H]⁺

¹H-NMR (400 MHz, DMSO-d6) δ[ppm]: 1.366 (1.17), 1.394 (1.79), 1.419 (1.29), 1.642 (1.06), 1.677 (1.17), 1.789 (1.56), 1.824 (2.35), 1.851 (3.79), 1.897 (5.83), 1.914 (5.08), 1.924 (4.96), 1.957 (1.33), 2.323 (0.88), 2.327 (1.25), 2.332 (0.88), 2.518 (3.58), 2.523 (2.50), 2.665 (0.90), 2.669 (1.27), 2.673 (0.88), 3.358 (1.54), 3.548 (1.23), 3.558 (1.17), 3.705 (1.02), 3.724 (1.02), 4.004 (1.33), 4.013 (1.42), 4.034 (1.60), 4.043 (1.52), 4.406 (1.33), 4.423 (1.54), 4.435 (1.25), 4.451 (1.25), 4.519 (1.27), 4.528 (1.44), 4.552 (1.56), 4.558 (1.60), 4.908 (1.21), 4.912 (1.21), 4.924 (1.73), 4.928 (1.71), 4.937 (1.08), 4.941 (1.02), 4.957 (1.50), 5.002 (1.46), 5.010 (1.54), 5.019 (2.02), 5.028 (1.29), 5.035 (0.98), 6.615 (2.98), 6.801 (6.10), 6.988 (2.63), 7.660 (2.02), 7.681 (2.29), 7.688 (2.15), 7.708 (2.04), 7.820 (16.00), 7.825 (3.40), 7.846 (3.04), 7.852 (3.06), 7.872 (2.90), 11.067 (3.23), 11.086 (3.02), 13.294 (3.31).

### Example 323

### N-[(trans)-2-(2-chloro-4,5-difluorophenyl)-1-oxo-2-azaspiro[5.5]undec-9-yl]-4-(morpholin-4-ylcarbonyl)-1H-imidazole-5-carboxamide

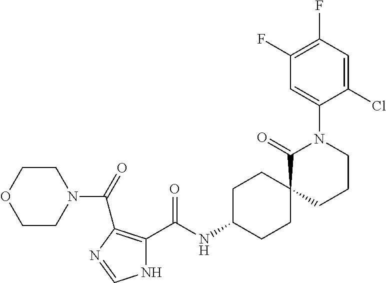 [SMILES: O=C(N[C@H]1CC[C@@]2(CCCN(c3cc(F)c(F)cc3Cl)C2=O)CC1)c1[nH]cnc1C(=O)N1CCOCC1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (88.0 mg, 162 μmol), morpholine (70.6 mg, 810 μmol) and triethylamine (68 μl) in tetrahydrofuran (1.6 ml) was stirred for 3 d at 50° C. For work-up, the reaction mixture was concentrated and the residue was purified by preparative HPLC (Method 9). The product crystalized from water/acetonitrile and was collected by filtration and dried under high vacuum to give the title compound (25 mg).

LC-MS (Method 1): Rₜ=1.05 min; MS (ESIpos): m/z=536.3 [M+H]⁺

¹H-NMR (400 MHz, DMSO-d6) δ [ppm]: 1.233 (1.40), 1.384 (2.09), 1.630 (2.99), 1.660 (2.84), 1.772 (3.39), 1.858 (5.13), 1.892 (10.42), 1.905 (10.07), 2.318 (0.95), 2.322 (2.09), 2.327 (2.99), 2.332 (2.19), 2.337 (0.95), 2.518 (10.52), 2.523 (6.98), 2.660 (0.95), 2.665 (2.14), 2.669 (2.94), 2.673 (2.09), 2.679 (0.90), 3.207 (0.60), 3.356 (3.24), 3.542 (3.14), 3.554 (2.99), 3.571 (2.69), 3.666 (16.00), 4.034 (3.74), 7.657 (3.74), 7.677 (4.34), 7.684 (4.09), 7.705 (3.69), 7.803 (5.83), 7.825 (5.43), 7.845 (5.73), 7.851 (5.88), 7.871 (5.38), 9.975 (1.84), 13.235 (2.29).

### Example 324

### N⁵-[(trans)-2-(2-chloro-4,5-difluorophenyl)-1-oxo-2-azaspiro[5.5]undec-9-yl]-N⁴-cyclopropyl-1H-imidazole-4,5-dicarboxamide

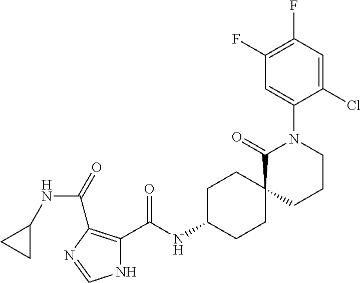 [SMILES: O=C(NC1CC1)c1nc[nH]c1C(=O)N[C@H]1CC[C@@]2(CCCN(c3cc(F)c(F)cc3Cl)C2=O)CC1]

A mixture of phenyl 5-{[2-(2-chloro-4,5-difluorophenyl)-1-oxo-2-azaspiro[5.5]undec-9-yl]carbamoyl}-1H-imidazole-4-carboxylate (isomer 1) (125 mg, 138 μmol) and cyclopropanamine (15.8 mg, 276 μmol, Cas No 765-30-0) and tetrahydrofuran (2.2 ml) was stirred for 17 h at room temperature. cyclopropanamine (15.8 mg, 276 μmol) was added and the mixture was stirred for 3 d at room temperature. For work-up, the reaction mixture was concentrated and the residue was purified by flash chromatography (25 g Snap Cartridge, dichloromethane/ethanol-gradient, 0%->19% ethanol) to give the title compound (32 mg).

LC-MS (Method 1): Rₜ=1.20 min; MS (ESIpos): m/z=506.2 [M+H]⁺

¹H-NMR (400 MHz, DMSO-d6) δ[ppm]: 0.497 (3.63), 0.687 (7.03), 0.742 (4.22), 1.232 (2.08), 1.437 (2.04), 1.643 (6.75), 1.790 (5.48), 1.910 (16.00), 2.323 (2.04), 2.327 (2.81), 2.331 (2.04), 2.523 (6.93), 2.540 (1.41), 2.665 (2.08), 2.669 (2.81), 2.674 (2.04), 2.852 (2.18), 3.347 (4.17), 3.360 (4.03), 3.547 (3.76), 3.749 (1.86), 5.759 (0.73), 7.657 (3.76), 7.678 (5.08), 7.684 (4.90), 7.704 (3.81), 7.805 (4.99), 7.825 (8.25), 7.845 (7.75), 7.852 (7.75), 7.871 (7.07), 8.453 (1.50), 8.569 (1.77), 11.185 (1.59), 11.261 (1.95), 13.229 (3.72).

### Example 325

### N-[(trans)-2-(2-chloro-4-fluorophenyl)-1-oxo-2-azaspiro[5.5]undec-9-yl]pyrazolo[1,5-a]pyrimidine-3-carboxamide

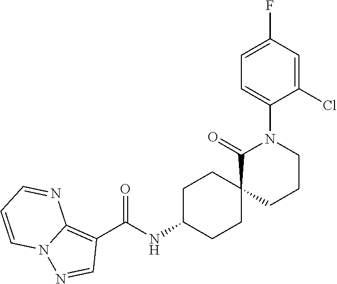 [SMILES: O=C(N[C@H]1CC[C@@]2(CCCN(c3ccc(F)cc3Cl)C2=O)CC1)c1cnn2cccnc12]

Benzotriazol-1-yl-oxytripyrrolidinophosphonium hexafluorophosphate (170 mg, 327 μmol) and N,N-diisopropylethylamine (210 μl, 1.2 mmol) were added to a mixture of pyrazolo[1,5-a]pyrimidine-3-carboxylic acid (50.0 mg, 297 μmol, Cas No 25940-35-6) and 9-amino-2-(2-chloro-4-fluorophenyl)-2-azaspiro[5.5]undecan-1-one (isomer 1) (127 mg, 327 μmol) in N,N-dimethylformamide (3.0 ml) and the mixture was stirred at room temperature for 5 d. For work-up, the reaction mixture was concentrated and the residue was purified by flash chromatography (25 g Isolera-cartridge dichloromethane/methanol-gradient 0%->10% methanol) The crude product was recrystallized from methanol to give the title compound (86.2 mg) with small impurities.

LC-MS (Method 2): Rₜ=1.10 min; MS (ESIpos): m/z=456.2 [M+H]⁺

¹H-NMR (400 MHz, CHLOROFORM-d) δ [ppm]: 0.000 (11.20), 1.124 (0.51), 1.141 (1.16), 1.158 (0.60), 1.349 (5.16), 1.366 (5.19), 1.386 (5.19), 1.389 (2.11), 1.402 (5.17), 1.408 (3.28), 1.426 (1.66), 1.455 (1.34), 1.464 (1.66), 1.471 (1.51), 1.480 (1.65), 1.493 (1.66), 1.498 (1.82), 1.508 (1.52), 1.526 (0.86), 1.532 (0.93), 1.549 (3.15), 1.663 (1.30), 1.670 (1.35), 1.698 (1.48), 1.705 (1.59), 1.734 (2.70), 1.741 (2.68), 1.750 (7.18), 1.758 (2.72), 1.767 (2.87), 1.786 (1.39), 1.793 (1.30), 1.821 (1.59), 1.828 (1.53), 1.883 (0.87), 1.899 (1.60), 1.907 (1.50), 1.914 (1.72), 1.922 (2.37), 1.928 (2.35), 1.949 (3.92), 1.959 (1.90), 1.963 (1.65), 1.976 (1.84), 1.988 (2.98), 1.997 (4.08), 2.011 (2.52), 2.021 (4.08), 2.032 (2.51), 2.048 (1.67), 2.084 (1.24), 2.094 (1.16), 2.118 (2.17), 2.128 (1.86), 2.140 (1.32), 2.151 (1.98), 2.161 (0.93), 2.174 (2.19), 2.184 (1.89), 2.208 (1.02), 2.218 (0.79), 3.070 (2.08), 3.073 (1.12), 3.079 (3.04), 3.081 (2.79), 3.087 (5.75), 3.090 (2.65), 3.092 (2.93), 3.096 (5.93), 3.100 (2.30), 3.104 (2.89), 3.112 (2.35), 3.402 (1.03), 3.407 (0.85), 3.419 (2.25), 3.436 (2.62), 3.449 (2.78), 3.462 (2.60), 3.478 (1.74), 3.491 (0.75), 3.621 (0.55), 3.631 (0.55), 4.006 (1.05), 4.016 (1.34), 4.026 (1.07), 4.036 (1.34), 4.046 (1.00), 6.910 (4.42), 6.921 (4.70), 6.928 (5.28), 6.933 (2.71), 6.938 (4.84), 6.945 (2.58), 6.948 (3.04), 6.952 (2.69), 6.955 (3.43), 6.968 (2.67), 6.974 (2.97), 7.122 (5.12), 7.127 (4.86), 7.134 (5.56), 7.136 (5.95), 7.144 (4.29), 7.147 (5.14), 7.154 (4.82), 7.158 (4.22), 7.705 (2.15), 7.724 (2.11), 8.556 (5.07), 8.560 (5.81), 8.567 (5.50), 8.571 (5.39), 8.618 (16.00), 8.699 (5.93), 8.703 (5.82), 8.717 (5.77), 8.721 (5.05).

### Example 326

### N-[(trans)-2-(2-chloro-4-fluorophenyl)-1-oxo-2-azaspiro[5.5]undec-9-yl]imidazo[1,2-b]pyridazine-3-carboxamide

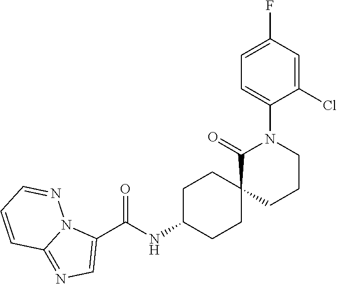 [SMILES: O=C(N[C@H]1CC[C@@]2(CCCN(c3ccc(F)cc3Cl)C2=O)CC1)c1cnc2cccnn12]

Benzotriazol-1-yl-oxytripyrrolidinophosphonium hexafluorophosphate (167 mg, 320 μmol) and N,N-diisopropylethylamine (200 μl, 1.2 mmol) were added to a mixture of imidazo[1,2-b]pyridazine-3-carboxylic acid (50.0 mg, 291 μmol, Cas No 1308384-58-8) and 9-amino-2-(2-chloro-4-fluorophenyl)-2-azaspiro[5.5]undecan-1-one (isomer 1) (124 mg, 320 μmol) in N,N-dimethylformamide (3.0 ml) and the mixture was stirred at room temperature for 5 d. For work-up, the reaction mixture was concentrated and the residue was purified by flash chromatography (25 g Isolera-cartridge dichloromethane/methanol-gradient 0%->10% methanol) The crude product was recrystallized from methanol to give the title compound (99.8 mg) with impurities.

LC-MS (Method 1): Rₜ=1.05 min; MS (ESIpos): m/z=456.2 [M+H]⁺

¹H-NMR (400 MHz, CHLOROFORM-d) δ [ppm]: 0.000 (9.65), 1.123 (0.73), 1.141 (1.57), 1.158 (0.76), 1.343 (11.70), 1.359 (11.85), 1.375 (12.66), 1.391 (12.20), 1.396 (8.33), 1.414 (3.41), 1.481 (1.00), 1.491 (1.30), 1.496 (1.18), 1.509 (1.25), 1.519 (1.33), 1.524 (1.42), 1.535 (1.18), 1.552 (0.64), 1.558 (0.63), 1.567 (0.58), 1.585 (4.96), 1.674 (0.98), 1.680 (1.00), 1.708 (1.27), 1.715 (1.28), 1.734 (6.05), 1.743 (6.33), 1.751 (16.00), 1.760 (6.32), 1.768 (6.35), 1.784 (0.85), 1.795 (1.18), 1.802 (0.99), 1.830 (1.22), 1.837 (1.17), 1.880 (0.50), 1.892 (0.76), 1.907 (1.18), 1.918 (1.37), 1.931 (1.95), 1.954 (3.26), 1.966 (1.40), 1.989 (1.90), 2.001 (2.46), 2.022 (2.26), 2.035 (2.31), 2.041 (1.95), 2.046 (2.02), 2.064 (0.99), 2.074 (0.91), 2.090 (1.11), 2.100 (0.86), 2.124 (1.59), 2.134 (1.39), 2.146 (1.02), 2.156 (1.34), 2.168 (0.73), 2.179 (1.64), 2.189 (1.39), 2.213 (0.76), 2.223 (0.59), 3.061 (4.95), 3.064 (2.25), 3.070 (6.78), 3.078 (13.59), 3.081 (6.77), 3.084 (6.23), 3.087 (12.94), 3.090 (6.13), 3.095 (6.67), 3.097 (4.50), 3.104 (4.57), 3.109 (2.14), 3.116 (0.82), 3.127 (0.57), 3.402 (0.93), 3.415 (0.64), 3.419 (1.06), 3.427 (1.45), 3.444 (2.13), 3.453 (1.82), 3.466 (2.06), 3.482 (1.31), 3.495 (0.55), 3.588 (0.42), 3.594 (0.93), 3.605 (0.98), 3.611 (1.24), 3.621 (1.25), 3.628 (0.96), 3.638 (0.89), 4.046 (0.79), 4.056 (1.01), 4.066 (0.80), 4.075 (1.02), 4.085 (0.77), 6.931 (1.72), 6.938 (1.61), 6.950 (1.88), 6.953 (2.05), 6.957 (2.03), 6.960 (2.29), 6.972 (1.70), 6.980 (1.88), 7.127 (3.86), 7.130 (3.92), 7.137 (3.46), 7.141 (3.78), 7.150 (4.68), 7.156 (4.74), 7.158 (4.09), 7.163 (3.16), 7.167 (3.71), 7.179 (3.56), 8.052 (3.58), 8.056 (3.62), 8.075 (3.28), 8.080 (3.28), 8.348 (1.57), 8.367 (1.56), 8.436 (3.20), 8.441 (3.23), 8.448 (3.78), 8.453 (12.91).

### Example 327

### N-[(trans)-2-(2-chloro-4-fluorophenyl)-1-oxo-2-azaspiro[5.5]undec-9-yl]-4-[(3-fluoroazetidin-1-yl)carbonyl]-1H-imidazole-5-carboxamide

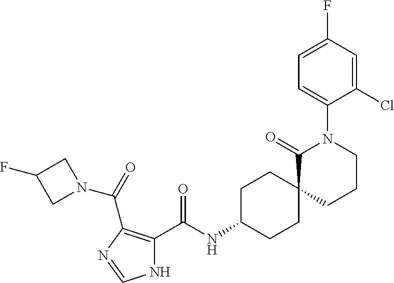 [SMILES: O=C(N[C@H]1CC[C@@]2(CCCN(c3ccc(F)cc3Cl)C2=O)CC1)c1[nH]cnc1C(=O)N1CC(F)C1]

A mixture of phenyl 5-{[2-(2-chloro-4-fluorophenyl)-1-oxo-2-azaspiro[5.5]undec-9-yl]carbamoyl}-1H-imidazole-4-carboxylate (isomer 1) (65.0 mg, 124 μmol), 3-fluoroazetidine hydrochloride (1:1) (27.6 mg, 248 μmol, Cas No 617718-46-4) and triethylamine (52 μl) in tetrahydrofuran (2.0 ml) was stirred for 1 d at room temperature. For work-up, the reaction mixture was concentrated and the residue was purified by flash chromatography (10 g NH2-cartridge, dichloromethane/methanol-gradient, 0%->10% methanol). The obtained product was recrystallized from diethyl ether give the title compound (47.4 mg).

LC-MS (Method 1): Rₜ=1.12 min; MS (ESIpos): m/z=506.2 [M+H]⁺

¹H-NMR (400 MHz, CHLOROFORM-d) δ [ppm]: 0.000 (4.30), 1.141 (0.89), 1.159 (0.46), 1.427 (0.80), 1.450 (1.43), 1.460 (2.16), 1.471 (1.49), 1.490 (2.34), 1.499 (1.64), 1.523 (1.08), 1.541 (16.00), 1.651 (1.38), 1.685 (1.55), 1.778 (1.32), 1.813 (1.65), 1.858 (1.46), 1.873 (1.81), 1.880 (2.31), 1.886 (2.39), 1.893 (2.37), 1.914 (2.86), 1.936 (2.76), 1.952 (3.33), 1.961 (3.92), 1.971 (4.42), 1.995 (2.89), 2.044 (1.19), 2.053 (1.14), 2.077 (2.04), 2.087 (1.79), 2.111 (1.70), 2.141 (1.91), 2.151 (1.73), 2.174 (0.86), 2.184 (0.71), 3.399 (0.78), 3.403 (0.87), 3.410 (1.86), 3.428 (3.14), 3.435 (2.25), 3.441 (2.49), 3.447 (3.19), 3.462 (2.01), 3.477 (0.76), 3.908 (1.12), 3.919 (1.41), 3.929 (1.08), 3.937 (1.39), 3.947 (1.08), 4.198 (0.66), 4.206 (0.89), 4.211 (0.73), 4.229 (0.97), 4.236 (1.23), 4.242 (1.03), 4.260 (0.74), 4.267 (0.89), 4.272 (0.72), 4.290 (0.99), 4.295 (1.22), 4.303 (0.94), 4.369 (0.88), 4.374 (0.86), 4.384 (0.97), 4.389 (0.97), 4.399 (0.66), 4.405 (0.69), 4.420 (1.55), 4.425 (1.01), 4.435 (0.98), 4.439 (0.97), 4.450 (0.66), 4.455 (0.69), 4.466 (0.66), 4.470 (0.66), 4.702 (0.73), 4.707 (0.85), 4.710 (0.91), 4.715 (0.78), 4.733 (0.99), 4.738 (1.15), 4.741 (1.21), 4.746 (1.06), 4.762 (0.81), 4.767 (0.88), 4.770 (0.91), 4.775 (0.76), 4.793 (0.98), 4.798 (1.14), 4.802 (1.14), 4.807 (0.96), 4.923 (0.88), 4.927 (0.88), 4.937 (1.10), 4.942 (1.03), 4.954 (0.69), 4.959 (0.73), 4.969 (0.88), 4.975 (1.43), 4.980 (1.00), 4.990 (1.04), 4.996 (0.98), 5.008 (0.73), 5.012 (0.76), 5.022 (0.75), 5.027 (0.74), 5.225 (0.95), 5.232 (1.17), 5.240 (1.59), 5.249 (1.04), 5.255 (0.79), 5.368 (0.87), 5.374 (1.12), 5.383 (1.56), 5.391 (1.08), 5.398 (0.88), 6.923 (1.69), 6.930 (2.14), 6.943 (2.44), 6.945 (2.69), 6.952 (2.92), 6.964 (2.16), 6.971 (2.33), 7.114 (3.76), 7.125 (5.60), 7.132 (4.91), 7.135 (3.86), 7.146 (4.53), 7.149 (4.01), 7.153 (4.48), 7.516 (12.61), 10.968 (2.95), 10.987 (2.89), 11.478 (1.91).

### Example 328

### N⁵-[(trans)-2-(2-chloro-4-fluorophenyl)-1-oxo-2-azaspiro[5.5]undec-9-yl]-N⁴-cyclopropyl-1H-imidazole-4,5-dicarboxamide

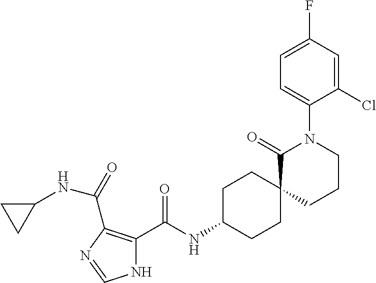 [SMILES: O=C(NC1CC1)c1nc[nH]c1C(=O)N[C@H]1CC[C@@]2(CCCN(c3ccc(F)cc3Cl)C2=O)CC1]

A mixture of phenyl 5-{[2-(2-chloro-4-fluorophenyl)-1-oxo-2-azaspiro[5.5]undec-9-yl]carbamoyl}-1H-imidazole-4-carboxylate (isomer 1) (65.0 mg, 124 μmol) cyclopropanamine (43 μl, 620 μmol, Cas No 765-30-0) in tetrahydrofuran (2 ml) was stirred for 1 d at room temperature. For work-up, the reaction mixture was concentrated and the residue was purified by flash chromatography (24 g NH2-cartridge, dichloromethane/methanol-gradient, 0%->10% methanol). The obtained product was recrystallized from diethyl ether give the title compound (28.7 mg).

LC-MS (Method 1): Rₜ=1.14 min; MS (ESIpos): m/z=488.3 [M+H]⁺

¹H-NMR (400 MHz, CHLOROFORM-d) δ [ppm]: 0.000 (1.80), 0.602 (0.62), 0.612 (0.92), 0.616 (1.82), 0.620 (2.24), 0.624 (2.02), 0.629 (2.71), 0.632 (2.98), 0.637 (1.68), 0.642 (2.26), 0.647 (1.64), 0.650 (1.46), 0.659 (0.71), 0.759 (0.67), 0.769 (0.89), 0.772 (1.40), 0.775 (1.21), 0.777 (1.91), 0.787 (0.95), 0.790 (1.88), 0.793 (1.21), 0.796 (1.28), 0.809 (1.16), 0.820 (1.05), 0.822 (1.85), 0.827 (2.31), 0.837 (1.17), 0.841 (2.43), 0.845 (1.61), 0.858 (0.61), 1.124 (0.74), 1.141 (1.59), 1.159 (0.75), 1.509 (0.78), 1.529 (16.00), 1.541 (0.87), 1.697 (0.78), 1.819 (0.78), 1.828 (0.85), 1.866 (1.14), 1.878 (0.92), 1.892 (1.80), 1.904 (2.12), 1.928 (2.35), 1.937 (2.34), 1.942 (2.27), 1.954 (2.12), 1.961 (2.24), 1.971 (2.03), 1.982 (1.79), 1.994 (1.63), 2.016 (0.80), 2.047 (0.74), 2.081 (0.85), 2.091 (1.07), 2.114 (0.88), 2.124 (1.03), 2.148 (0.93), 2.156 (1.14), 2.820 (0.52), 2.830 (0.73), 2.838 (1.15), 2.848 (1.18), 2.856 (0.96), 2.866 (0.94), 2.873 (0.96), 2.883 (0.92), 3.403 (1.09), 3.415 (1.07), 3.420 (1.30), 3.428 (1.38), 3.433 (1.31), 3.441 (1.64), 3.453 (1.98), 3.468 (1.22), 3.925 (0.73), 6.924 (1.35), 6.931 (1.52), 6.944 (1.67), 6.947 (1.79), 6.951 (1.84), 6.954 (2.02), 6.966 (1.62), 6.973 (1.73), 7.117 (3.12), 7.126 (3.22), 7.131 (3.91), 7.133 (3.68), 7.139 (2.94), 7.147 (3.19), 7.153 (5.01), 7.415 (0.85), 7.436 (0.83), 7.491 (4.91), 7.513 (4.12), 7.599 (1.08), 7.609 (1.06), 10.936 (1.00), 10.955 (0.99).

### Example 329

### N⁵-[(trans)-2-(2-chloro-4-fluorophenyl)-1-oxo-2-azaspiro[5.5]undec-9-yl]-N⁴-(propan-2-yl)-1H-imidazole-4,5-dicarboxamide

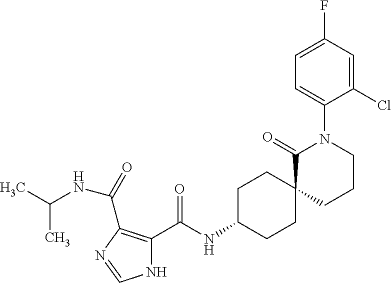 [SMILES: CC(C)NC(=O)c1nc[nH]c1C(=O)N[C@H]1CC[C@@]2(CCCN(c3ccc(F)cc3Cl)C2=O)CC1]

A mixture of phenyl 5-{[2-(2-chloro-4-fluorophenyl)-1-oxo-2-azaspiro[5.5]undec-9-yl]carbamoyl}-1H-imidazole-4-carboxylate (isomer 1) (65.0 mg, 124 μmol), propan-2-amine (53 μl, 620 μmol, Cas No 75-31-0) in tetrahydrofuran (2.0 ml) was stirred for 5 h at room temperature. For work-up, the reaction mixture was concentrated and the residue was purified by flash chromatography (24 g NH2-cartridge, dichloromethane/methanol-gradient, 0%->10% methanol). The obtained product was recrystallized from diethyl ether give the title compound (36.6 mg).

LC-MS (Method 1): Rₜ=1.21 min; MS (ESIpos): m/z=490.3 [M+H]⁺

¹H-NMR (400 MHz, CHLOROFORM-d) δ [ppm]: 0.000 (0.68), 1.124 (7.60), 1.141 (16.00), 1.159 (7.38), 1.212 (9.32), 1.229 (9.67), 1.237 (7.44), 1.253 (7.27), 1.551 (4.91), 1.897 (0.92), 1.904 (0.94), 1.928 (1.22), 1.942 (1.29), 1.961 (1.24), 1.975 (1.17), 1.984 (1.24), 1.994 (1.11), 3.385 (2.61), 3.403 (7.48), 3.420 (7.35), 3.438 (3.12), 3.453 (1.03), 6.924 (0.83), 6.931 (0.94), 6.944 (1.00), 6.946 (1.11), 6.951 (1.09), 6.954 (1.19), 6.966 (0.98), 6.973 (1.07), 7.118 (1.50), 7.127 (1.57), 7.133 (2.57), 7.140 (1.34), 7.147 (1.56), 7.154 (2.76), 7.434 (1.02), 7.498 (4.49), 7.501 (3.42).

### Example 330

### N-[(trans)-2-(2-chloro-4-fluorophenyl)-1-oxo-2-azaspiro[4.5]dec-8-yl]-5-(difluoromethyl)pyrazolo[1,5-a]pyrimidine-3-carboxamide

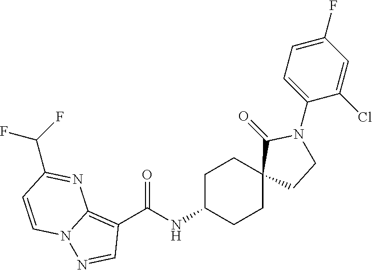 [SMILES: O=C(N[C@H]1CC[C@@]2(CCN(c3ccc(F)cc3Cl)C2=O)CC1)c1cnn2ccc(C(F)F)nc12]

To a mixture of N-[2-(2-chloro-4-fluorophenyl)-1-oxo-2-azaspiro[4.5]dec-8-yl]pyrazolo[1,5-a]pyrimidine-3-carboxamide (isomer 1) (200 mg, 453 μmol) and zinc bis(difluoromethanesulfinate) (268 mg, 905 μmol) in DMSO (2.6 ml) was added trifluoroacetic acid (37 μl, 480 μmol). The mixture was cooled to 0′C and tert-butyl hydroperoxide (190 μl, 70% purity, 0.0 μmol) was added. The reaction was stirred in a closed microwave vial for 16 hours at 50° C. For work-up the mixture was diluted with ethyl acetate and the mixture was poured into a solution of EDTA/saturated sodium bicarbonate solution (40 mL, 18 g EDTA in 150 mL sat sodium bicarbonate solution). The aqueous phase was extracted with ethyl acetate and the combined organic phases were washed with brine. The organic phase was filtrated over a hydrophobic filter and concentrated. The residue was purified by preparative HPLC [Instrument: Waters Autopurificationsystem; column: Waters XBrigde C18 5p 100×30 mm; Eluent A: water+0.2 Vol-% aqueous ammonia (32%), Eluent B: acetonitrile; gradient: 0.00-0.50 min 19% B (25->70 mL/min), 0.51-5.50 min 39-65% B (70 mL/min), DAD scan: 210-400 nm] to give the title compound (19 mg).

LC-MS (Method 2): Rₜ=1.15 min; MS (ESIpos): m/z=492 [M+H]⁺

¹H-NMR (400 MHz, DMSO-d6) δ [ppm]: 0.967 (1.62), 1.107 (16.00), 1.144 (0.97), 1.176 (0.48), 1.209 (0.65), 1.232 (0.48), 1.457 (0.40), 1.480 (0.81), 1.489 (0.81), 1.510 (1.37), 1.531 (1.05), 1.560 (0.57), 1.613 (0.48), 1.675 (3.88), 1.683 (4.53), 1.698 (2.91), 1.705 (2.91), 1.957 (1.37), 1.965 (1.45), 1.988 (1.37), 1.997 (1.29), 2.150 (2.51), 2.167 (4.53), 2.185 (2.59), 2.337 (0.73), 2.518 (9.21), 2.523 (6.55), 2.633 (0.57), 2.674 (1.78), 2.679 (0.81), 3.298 (0.81), 3.351 (1.45), 3.611 (2.75), 3.629 (4.77), 3.646 (2.59), 3.838 (0.57), 3.848 (0.73), 3.857 (0.57), 3.867 (0.73), 3.877 (0.57), 4.192 (0.97), 7.289 (1.29), 7.296 (1.45), 7.311 (1.94), 7.318 (2.10), 7.331 (1.54), 7.338 (1.70), 7.466 (2.59), 7.480 (2.67), 7.488 (2.26), 7.502 (2.02), 7.544 (1.37), 7.579 (4.69), 7.586 (3.47), 7.592 (2.83), 7.601 (2.91), 7.608 (2.75), 7.675 (3.15), 7.805 (1.29), 7.820 (2.10), 7.840 (2.02), 8.704 (10.26), 8.711 (0.57), 8.971 (4.36), 8.982 (3.88).

### Example 331

### N-[(trans)-2-(2-chloro-4-fluorophenyl)-1-oxo-2-azaspiro[4.5]dec-8-yl]-5,7-bis(difluoromethyl)pyrazolo[1,5-a]pyrimidine-3-carboxamide

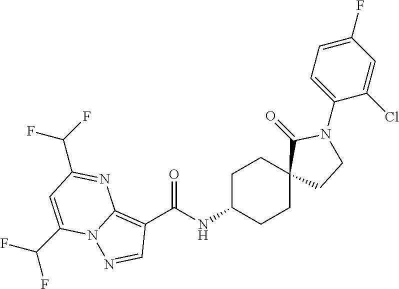 [SMILES: O=C(N[C@H]1CC[C@@]2(CCN(c3ccc(F)cc3Cl)C2=O)CC1)c1cnn2c(C(F)F)cc(C(F)F)nc12]

The title product (10 mg) was isolated as a side product in the synthesis of N-[(trans)-2-(2-chloro-4-fluorophenyl)-1-oxo-2-azaspiro[4.5]dec-8-yl]-5-(difluoromethyl)pyrazolo[1,5-a]pyrimidine-3-carboxamide.

LC-MS (Method 2): Rₜ=1.25 min; MS (ESIpos): m/z=542 [M+H]⁺

¹H-NMR (600 MHz, DMSO-d6) δ[ppm]: 0.000 (2.08), 1.167 (1.13), 1.179 (2.26), 1.191 (1.02), 1.471 (0.98), 1.490 (1.79), 1.499 (2.08), 1.508 (2.30), 1.518 (2.08), 1.527 (1.31), 1.539 (1.02), 1.626 (0.47), 1.669 (0.62), 1.700 (8.31), 1.706 (9.08), 1.717 (6.05), 1.721 (4.56), 1.739 (0.44), 1.867 (0.44), 1.990 (4.41), 2.000 (2.92), 2.006 (3.03), 2.013 (2.04), 2.021 (2.88), 2.027 (2.62), 2.117 (0.47), 2.129 (0.84), 2.140 (0.55), 2.154 (5.03), 2.166 (9.11), 2.177 (5.14), 2.387 (0.55), 2.390 (0.73), 2.393 (0.51), 2.521 (1.79), 2.524 (1.68), 2.527 (1.31), 2.615 (0.51), 2.618 (0.69), 2.621 (0.51), 3.316 (0.44), 3.323 (1.79), 3.374 (0.44), 3.621 (0.77), 3.632 (5.98), 3.643 (9.55), 3.654 (5.07), 3.840 (0.62), 3.846 (0.69), 3.852 (1.17), 3.859 (1.42), 3.865 (1.20), 3.871 (1.42), 3.877 (1.09), 3.884 (0.62), 3.890 (0.55), 4.024 (0.98), 4.036 (0.87), 5.746 (1.57), 7.140 (3.10), 7.229 (6.78), 7.290 (1.97), 7.295 (2.04), 7.304 (3.57), 7.309 (3.43), 7.319 (4.30), 7.323 (2.44), 7.470 (4.19), 7.480 (4.26), 7.485 (3.54), 7.495 (3.17), 7.560 (0.66), 7.565 (4.30), 7.571 (3.97), 7.574 (0.91), 7.580 (4.12), 7.585 (3.94), 7.617 (2.19), 7.703 (4.85), 7.764 (10.46), 7.790 (1.90), 7.826 (3.94), 7.839 (3.79), 7.954 (1.02), 8.309 (2.59), 8.832 (16.00).

### Example 332

### N-[(cis)-2-(2-chloro-4-fluorophenyl)-1-oxo-2-azaspiro[4.5]dec-8-yl]-1H-pyrrolo[3,2-b]pyridine-3-carboxamide

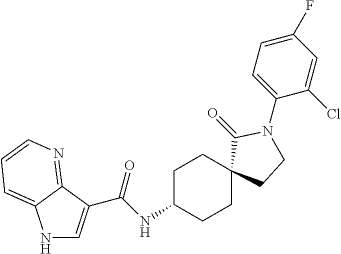 [SMILES: O=C(N[C@H]1CC[C@]2(CCN(c3ccc(F)cc3Cl)C2=O)CC1)c1c[nH]c2cccnc12]

To a solution of 8-amino-2-(2-chloro-4-fluorophenyl)-2-azaspiro[4.5]decan-1-one (isomer 2) (80.0 mg, 270 μmol) in DMF (2.7 ml) was added PYBOP (1H-benzotriazol-1-yloxy)[tri(pyrrolidin-1-yl)]phosphonium hexafluorophosphate (154 mg, 297 μmol), N,N-diisopropylethylamine (190 μl, 1.1 mmol) and 1H-pyrrolo[3,2-b]pyridine-3-carboxylic acid (43.7 mg, 270 μmol) and the reaction was stirred for 16 hours at room temperature. For work-up, brine (45 ml) was added and the resulting precipitate was filtrated. The residue was dissolved in DMSO and the crude product was purified by HPLC (Method 8) to give after trituration with water the title product (19 mg)

LC-MS (Method 6): Rₜ=0.88 min; MS (ESIpos): m/z=441 [M+H]⁺

¹H-NMR (400 MHz, DMSO-d6) δ[ppm]: 1.233 (1.03), 1.525 (3.09), 1.534 (3.20), 1.546 (4.23), 1.559 (3.36), 1.568 (3.63), 1.578 (2.98), 1.765 (2.12), 1.777 (2.33), 1.787 (4.34), 1.797 (4.50), 1.810 (3.63), 1.820 (4.01), 1.889 (2.66), 1.899 (3.36), 1.907 (4.61), 1.926 (3.42), 1.940 (2.71), 1.960 (5.37), 1.970 (3.25), 1.983 (3.58), 1.993 (5.26), 2.003 (3.31), 2.016 (2.93), 2.025 (2.22), 2.078 (6.40), 2.095 (12.58), 2.112 (6.62), 2.318 (1.03), 2.323 (2.33), 2.327 (3.25), 2.332 (2.28), 2.337 (0.98), 2.518 (13.02), 2.523 (9.27), 2.540 (0.49), 2.660 (1.03), 2.665 (2.39), 2.669 (3.31), 2.674 (2.28), 2.679 (0.98), 3.611 (8.19), 3.628 (15.02), 3.644 (7.70), 4.076 (2.28), 4.085 (2.33), 4.093 (2.22), 7.221 (9.22), 7.233 (8.57), 7.242 (8.89), 7.254 (9.49), 7.287 (4.07), 7.294 (4.61), 7.308 (5.75), 7.316 (6.94), 7.329 (4.94), 7.336 (5.10), 7.478 (8.46), 7.492 (8.89), 7.500 (7.43), 7.514 (7.00), 7.575 (8.41), 7.582 (8.57), 7.596 (8.46), 7.603 (8.03), 7.898 (10.68), 7.902 (11.01), 7.919 (10.14), 7.922 (9.93), 8.148 (16.00), 8.156 (15.84), 8.445 (10.25), 8.448 (11.12), 8.456 (10.47), 8.460 (9.27), 9.034 (3.42), 9.053 (3.36), 11.927 (4.01).

### Example 333

### N-[(cis)-2-(2-chloro-4-fluorophenyl)-1-oxo-2-azaspiro[4.5]dec-8-yl]-7-(difluoromethyl)-5-methylpyrazolo[1,5-a]pyrimidine-3-carboxamide

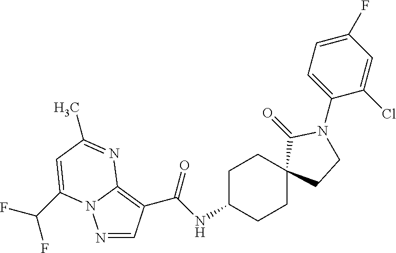 [SMILES: Cc1cc(C(F)F)n2ncc(C(=O)N[C@H]3CC[C@]4(CCN(c5ccc(F)cc5Cl)C4=O)CC3)c2n1]

To a solution of 8-amino-2-(2-chloro-4-fluorophenyl)-2-azaspiro[4.5]decan-1-one (isomer 2) (70.0 mg, 236 μmol) in DMF (2.4 ml) was added PYBOP (1H-benzotriazol-1-yloxy)[tri(pyrrolidin-1-yl)]phosphonium hexafluorophosphate (135 mg, 259 μmol), N,N-diisopropylethylamine (160 μl, 940 μmol) 7-(difluoromethyl)-5-methylpyrazolo[1,5-a]pyrimidine-3-carboxylic acid (53.6 mg, 236 μmol) and the reaction was stirred for 16 hours at room temperature. For work-up sodium chloride solution (45 ml) was added and the resulting precipitate was filtrated. The residue was dissolved in DMSO and the crude product was purified by HPLC (Method 8) to give after trituration with water the title product (27 mg)

LC-MS (Method 2): Rₜ=1.22 min; MS (ESIpos): m/z=506 [M+H]⁺

¹H-NMR (400 MHz, DMSO-d6) δ[ppm]: 1.548 (1.22), 1.559 (0.79), 1.571 (0.78), 1.582 (1.36), 1.803 (2.56), 1.814 (2.87), 1.924 (0.81), 1.942 (1.16), 1.959 (1.09), 1.976 (1.13), 1.995 (0.48), 2.074 (0.51), 2.124 (1.50), 2.141 (2.91), 2.158 (1.56), 2.323 (0.40), 2.327 (0.56), 2.331 (0.40), 2.518 (1.80), 2.523 (1.17), 2.540 (4.17), 2.665 (0.40), 2.669 (0.56), 2.755 (16.00), 3.616 (1.87), 3.633 (3.43), 3.650 (1.79), 4.200 (0.62), 4.210 (0.68), 4.220 (0.60), 7.290 (0.83), 7.298 (0.93), 7.312 (1.37), 7.319 (1.56), 7.332 (1.00), 7.340 (1.10), 7.479 (1.87), 7.493 (2.12), 7.500 (2.11), 7.515 (5.03), 7.579 (1.83), 7.586 (1.91), 7.600 (1.89), 7.607 (1.85), 7.628 (2.38), 7.759 (0.93), 8.198 (0.54), 8.444 (0.80), 8.465 (0.78), 8.596 (7.47).

### Example 334

### N⁵-[(trans)-2-(2-chloro-4-fluoro-5-methylphenyl)-1-oxo-2-azaspiro[4.5]dec-8-yl]-N⁴-(2-methoxyethyl)-1H-imidazole-4,5-dicarboxamide

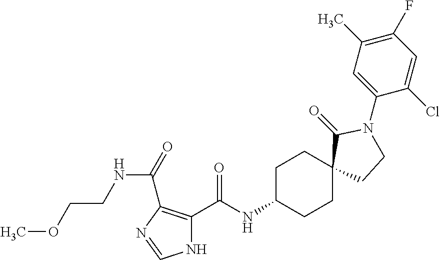 [SMILES: COCCNC(=O)c1nc[nH]c1C(=O)N[C@H]1CC[C@@]2(CCN(c3cc(C)c(F)cc3Cl)C2=O)CC1]

A mixture of phenyl 5-{[2-(2-chloro-4-fluoro-5-methylphenyl)-1-oxo-2-azaspiro[4.5]dec-8-yl]carbamoyl}-1H-imidazole-4-carboxylate (isomer 1) (150 mg, 92% purity, 263 μmol) and 2-methoxyethanamine (98.7 mg, 1.31 mmol) in THF (4.3 ml) was stirred for 20 hours at room temperature. For work-up a sodium chloride solution was added and the mixture was extracted with ethyl acetate. The combined organic phases filtrated over a hydrophobic filter and concentrated. The residue was purified by flash chromatography (dichloromethane/ethanol-gradient, 0%->17% ethanol) to give the title compound (23 mg).

LC-MS (Method 2): Rₜ=1.09 min; MS (ESIpos): m/z=506 [M+H]⁺

¹H-NMR (500 MHz, DMSO-d6) δ[ppm]: 1.656 (0.89), 1.987 (0.95), 2.129 (0.54), 2.222 (2.91), 2.225 (2.83), 3.273 (13.59), 3.331 (16.00), 3.588 (0.75), 3.602 (1.32), 3.616 (0.72), 7.367 (0.43), 7.382 (0.43), 7.487 (1.14), 7.506 (1.11), 7.822 (1.38), 13.238 (0.51).

### Example 335

### N-[(cis)-2-(2-chloro-4-fluorophenyl)-1-oxo-2-azaspiro[4.5]dec-8-yl]-5-methylpyrazolo[1,5-a]pyrimidine-3-carboxamide

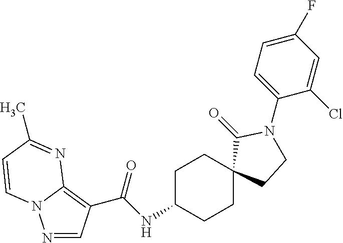 [SMILES: Cc1ccn2ncc(C(=O)N[C@H]3CC[C@]4(CCN(c5ccc(F)cc5Cl)C4=O)CC3)c2n1]

To a solution of 8-amino-2-(2-chloro-4-fluorophenyl)-2-azaspiro[4.5]decan-1-one (isomer 2) (75.0 mg, 253 μmol) in DMF (2.6 ml) was added PYBOP (145 mg, 278 μmol), N,N-diisopropylethylamine (180 μl, 1.0 mmol) and 5-methylpyrazolo[1,5-a]pyrimidine-3-carboxylic acid (44.8 mg, 253 μmol, CAS 62908-85-4) and the reaction was stirred for 16 hours at room temperature. For work-up sodium chloride solution (45 ml) was added and the resulting precipitate was filtrated. The residue was recrystallized from DMSO to give the title product (47 mg)

LC-MS (Method 1): Rₜ=1.10 min; MS (ESIpos): m/z=456 [M+H]⁺

¹H-NMR (400 MHz, DMSO-d6) δ [ppm]: 1.540 (1.03), 1.574 (1.18), 1.771 (1.15), 1.782 (2.12), 1.794 (2.16), 1.802 (2.13), 1.812 (1.22), 1.943 (1.11), 2.124 (1.35), 2.141 (2.64), 2.158 (1.41), 2.539 (1.85), 2.679 (16.00), 3.613 (1.68), 3.630 (3.07), 3.648 (1.60), 7.138 (3.31), 7.157 (3.33), 7.289 (0.72), 7.295 (0.90), 7.310 (1.21), 7.317 (1.29), 7.331 (0.94), 7.338 (1.08), 7.476 (1.72), 7.491 (1.79), 7.499 (1.50), 7.513 (1.40), 7.577 (1.66), 7.584 (1.66), 7.598 (1.65), 7.606 (1.61), 8.470 (7.39), 9.144 (3.65), 9.162 (3.51).

### Example 336

### N-[(cis)-2-(2-chloro-4-fluorophenyl)-1-oxo-2-azaspiro[4.5]dec-8-yl]-7-methylpyrazolo[1,5-a]pyrimidine-3-carboxamide

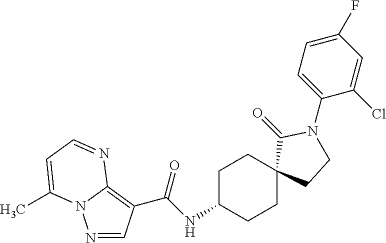 [SMILES: Cc1ccnc2c(C(=O)N[C@H]3CC[C@]4(CCN(c5ccc(F)cc5Cl)C4=O)CC3)cnn12]

To a solution of 8-amino-2-(2-chloro-4-fluorophenyl)-2-azaspiro[4.5]decan-1-one (isomer 2) (75.0 mg, 253 μmol) in DMF (2.6 ml) was added PYBOP (145 mg, 278 μmol), N,N-diisopropylethylamine (180 μl, 1.0 mmol) 7-methylpyrazolo[1,5-a]pyrimidine-3-carboxylic acid (44.8 mg, 253 μmol) and the reaction was stirred for 16 hours at room temperature. For work-up sodium chloride solution (45 ml) was added and the resulting precipitate was filtrated. The residue was dissolved in DMSO and the crude product was purified by HPLC (Method 8) to give the title product (62 mg)

LC-MS (Method 4): Rₜ=1.09 min; MS (ESIpos): m/z=456 [M+H]⁺

¹H-NMR (400 MHz, DMSO-d6) δ[ppm]: 1.527 (1.10), 1.537 (1.18), 1.548 (1.67), 1.560 (1.22), 1.573 (1.26), 1.580 (1.05), 1.788 (1.53), 1.798 (1.62), 1.809 (1.45), 1.821 (1.22), 1.885 (0.98), 1.895 (1.20), 1.903 (1.74), 1.921 (2.07), 1.929 (2.86), 1.944 (1.76), 1.954 (1.98), 1.964 (1.16), 1.976 (1.05), 2.066 (2.21), 2.074 (1.09), 2.083 (4.36), 2.100 (2.27), 2.540 (1.21), 2.795 (15.89), 2.797 (16.00), 3.607 (2.79), 3.624 (5.14), 3.641 (2.64), 4.054 (0.80), 4.063 (0.84), 4.071 (0.80), 7.222 (2.73), 7.224 (2.60), 7.233 (2.63), 7.235 (2.80), 7.285 (1.22), 7.292 (1.40), 7.306 (1.98), 7.314 (2.16), 7.327 (1.50), 7.334 (1.62), 7.474 (2.73), 7.488 (2.87), 7.496 (2.40), 7.511 (2.22), 7.572 (2.58), 7.580 (2.73), 7.594 (2.65), 7.601 (2.65), 8.144 (1.38), 8.164 (1.34), 8.196 (0.67), 8.589 (11.71), 8.690 (5.58), 8.701 (5.32).

### Example 337

### N-[(cis)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide

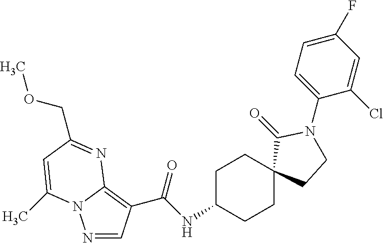 [SMILES: COCc1cc(C)n2ncc(C(=O)N[C@H]3CC[C@]4(CCN(c5ccc(F)cc5Cl)C4=O)CC3)c2n1]

To a solution of 8-amino-2-(2-chloro-4-fluorophenyl)-2-azaspiro[4.5]decan-1-one (isomer 2) (70.0 mg, 236 μmol) in DMF (2.4 ml) was added PYBOP (135 mg, 259 μmol), N,N-diisopropylethylamine (160 μl, 940 μmol) 5-(methoxymethyl)-7-methylpyrazolo[1,5-a]pyrimidine-3-carboxylic acid (52.2 mg, 236 μmol) and the reaction was stirred for 16 hours at room temperature. For work-up sodium chloride solution (45 ml) was added and the mixture was extracted with ethyl acetate. The combined organic phases filtrated over a hydrophobic filter and concentrated. The residue was purified by flash chromatography (dichloromethane/ethyl acetate-gradient, 0%->100% ethyl acetate) to give the title compound (21 mg).

LC-MS (Method 1): Rₜ=1.17 min; MS (ESIpos): m/z=500 [M+H]⁺

¹H-NMR (400 MHz, DMSO-d6) δ[ppm]: 1.788 (1.31), 1.799 (2.14), 1.811 (2.43), 1.822 (1.19), 1.987 (1.16), 2.116 (1.15), 2.133 (2.28), 2.151 (1.22), 2.801 (8.45), 2.803 (8.83), 3.330 (16.00), 3.615 (1.44), 3.632 (2.67), 3.649 (1.38), 4.674 (9.07), 7.245 (2.64), 7.247 (2.76), 7.290 (0.66), 7.297 (0.74), 7.312 (1.07), 7.319 (1.18), 7.333 (0.82), 7.340 (0.87), 7.483 (1.41), 7.498 (1.52), 7.506 (1.26), 7.520 (1.17), 7.579 (1.42), 7.586 (1.46), 7.601 (1.51), 7.608 (1.41), 8.554 (6.18).

### Example 338

### N⁵-[(cis)-2-(2-chloro-4-fluorophenyl)-1-oxo-2-azaspiro[4.5]dec-8-yl]-N⁴-cyclopropyl-1H-imidazole-4,5-dicarboxamide

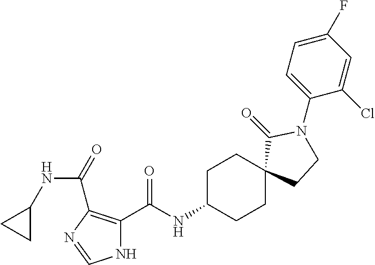 [SMILES: O=C(NC1CC1)c1nc[nH]c1C(=O)N[C@H]1CC[C@]2(CCN(c3ccc(F)cc3Cl)C2=O)CC1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 2) (100 mg, 196 μmol) and cyclopropanamine (22.3 mg, 391 μmol) in THF (1.9 ml) was added triethylamine (82 μl, 590 μmol) and the reaction was stirred at room temperature for 16 hours. For work up the reaction was concentrated and the residue was purified by HPLC (Method 9) to give the title product (50 mg)

LC-MS (Method 3): Rₜ=1.15 min; MS (ESIpos): m/z=474 [M+H]⁺

¹H-NMR (400 MHz, DMSO-d6) δ[ppm]: 0.678 (4.71), 1.232 (0.86), 1.488 (2.78), 1.499 (3.37), 1.509 (4.89), 1.522 (3.40), 1.534 (3.58), 1.742 (4.19), 1.751 (4.25), 1.764 (3.55), 1.904 (3.82), 1.922 (6.88), 1.936 (6.33), 1.948 (6.30), 1.967 (3.98), 2.049 (6.82), 2.065 (13.67), 2.082 (7.19), 2.337 (0.58), 2.518 (5.32), 2.523 (3.73), 2.873 (2.23), 3.596 (8.50), 3.613 (16.00), 3.630 (8.29), 3.925 (2.05), 7.282 (4.44), 7.289 (4.93), 7.302 (5.72), 7.304 (6.24), 7.311 (7.59), 7.324 (5.29), 7.331 (5.57), 7.471 (9.27), 7.485 (9.61), 7.493 (8.05), 7.508 (7.46), 7.570 (8.84), 7.578 (9.42), 7.592 (9.15), 7.599 (9.24), 7.789 (10.40), 8.532 (0.98), 11.255 (0.95), 13.205 (1.47).

### Example 339

### N⁵-[(cis)-2-(2-chloro-4-fluorophenyl)-1-oxo-2-azaspiro[4.5]dec-8-yl]-N⁴-ethyl-1H-imidazole-4,5-dicarboxamide

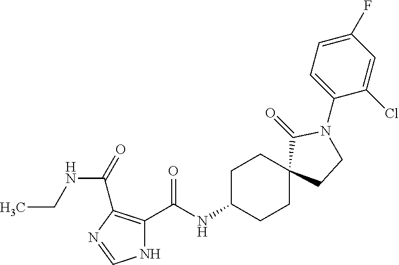 [SMILES: CCNC(=O)c1nc[nH]c1C(=O)N[C@H]1CC[C@]2(CCN(c3ccc(F)cc3Cl)C2=O)CC1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 2) (100 mg, 196 μmol) and ethanamine (17.6 mg, 391 μmol) in THF (1.9 ml) was added triethylamine (82 μl, 590 μmol) and the reaction was stirred at room temperature for 16 hours. For work up the reaction was concentrated and the residue was purified by HPLC (Method 9) to give the title product (47 mg)

LC-MS (Method 6): Rₜ=1.08 min; MS (ESIpos): m/z=462 [M+H]⁺

¹H-NMR (400 MHz, DMSO-d6) δ[ppm]: 1.086 (3.58), 1.103 (6.62), 1.120 (4.31), 1.232 (1.00), 1.499 (2.96), 1.523 (2.33), 1.718 (1.63), 1.730 (1.90), 1.739 (3.58), 1.749 (3.61), 1.762 (2.85), 1.772 (2.66), 1.933 (4.37), 2.049 (5.13), 2.066 (10.33), 2.083 (5.42), 2.518 (5.26), 2.523 (3.53), 3.275 (3.06), 3.291 (4.42), 3.308 (3.58), 3.594 (6.24), 3.611 (11.69), 3.628 (5.94), 3.924 (1.38), 7.282 (3.17), 7.289 (3.53), 7.302 (4.37), 7.309 (4.72), 7.324 (3.66), 7.331 (4.34), 7.469 (6.56), 7.483 (6.81), 7.491 (5.64), 7.506 (5.23), 7.570 (6.62), 7.578 (7.11), 7.592 (6.86), 7.599 (6.92), 7.796 (16.00), 8.599 (1.11), 11.326 (1.14), 13.170 (1.27).

### Example 340

### N-[(cis)-2-(2-chloro-4-fluorophenyl)-1-oxo-2-azaspiro[4.5]dec-8-yl]-4-[(3-fluoroazetidin-1-yl)carbonyl]-1H-imidazole-5-carboxamide

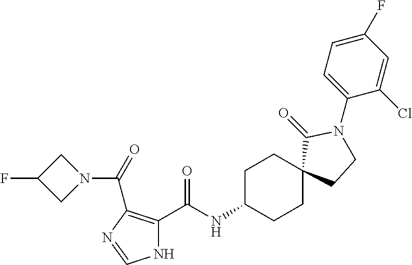 [SMILES: O=C(N[C@H]1CC[C@]2(CCN(c3ccc(F)cc3Cl)C2=O)CC1)c1[nH]cnc1C(=O)N1CC(F)C1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 2) (100 mg, 196 μmol) and 3-fluoroazetidine (980 μl, 390 μmol) in THF (1.9 ml) was added triethylamine (82 μl, 590 μmol) and the reaction was stirred at room temperature for 16 hours. For work up the reaction was concentrated and the residue was purified by HPLC (Method 9) to give the title product (49 mg)

LC-MS (Method 6): Rₜ=1.03 min; MS (ESIpos): m/z=492 [M+H]⁺

1H-NMR (400 MHz, DMSO-d6): δ [ppm]=13.26 (br s, 1H), 11.15 (d, 1H), 7.82 (s, 1H), 7.58 (dd, 1H), 7.48 (dd, 1H), 7.31 (td, 1H), 5.61-5.24 (m, 1H), 5.03-4.82 (m, 1H), 4.70-4.51 (m, 1H), 4.48-4.27 (m, 1H), 4.19-3.99 (m, 1H), 3.98-3.84 (m, 1H), 3.61 (t, 2H), 2.11-2.02 (m, 2H), 1.99-1.85 (m, 4H), 1.74 (br s, 2H), 1.55-1.40 (m, 2H).

### Example 341

### N⁵-[(cis)-2-(2-chloro-4-fluorophenyl)-1-oxo-2-azaspiro[4.5]dec-8-yl]-N⁴-(2-methoxyethyl)-1H-imidazole-4,5-dicarboxamide

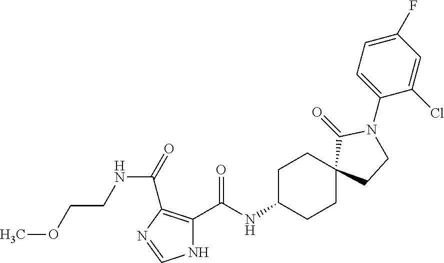 [SMILES: COCCNC(=O)c1nc[nH]c1C(=O)N[C@H]1CC[C@]2(CCN(c3ccc(F)cc3Cl)C2=O)CC1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 2) (100 mg, 196 μmol) and 2-methoxyethanamine (29.4 mg, 391 μmol) in THF (1.9 ml) was added triethylamine (82 μl, 590 μmol) and the reaction was stirred at room temperature for 16 hours. For work up the reaction was concentrated and the residue was purified by HPLC (Method 9) to give the title product (49 mg)

LC-MS (Method 6): Rₜ=1.07 min; MS (ESIpos): m/z=492 [M+H]⁺

¹H-NMR (400 MHz, DMSO-d6) δ[ppm]: 1.501 (1.31), 1.525 (1.04), 1.742 (1.58), 1.753 (1.55), 1.765 (1.24), 1.775 (1.15), 1.931 (1.88), 2.051 (2.22), 2.068 (4.39), 2.085 (2.31), 2.518 (1.89), 2.523 (1.26), 3.254 (16.00), 3.456 (11.66), 3.593 (2.66), 3.610 (5.00), 3.627 (2.59), 7.281 (1.41), 7.289 (1.45), 7.303 (2.07), 7.309 (2.06), 7.323 (1.64), 7.331 (1.80), 7.468 (2.71), 7.482 (2.87), 7.490 (2.40), 7.504 (2.24), 7.570 (3.00), 7.577 (3.13), 7.591 (3.05), 7.598 (2.98), 7.808 (9.38).

### Example 342

### N⁵-[(cis)-2-(2-chloro-4-fluorophenyl)-1-oxo-2-azaspiro[4.5]dec-8-yl]-1H-imidazole-4,5-dicarboxamide

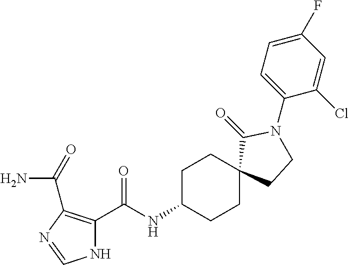 [SMILES: NC(=O)c1nc[nH]c1C(=O)N[C@H]1CC[C@]2(CCN(c3ccc(F)cc3Cl)C2=O)CC1]

A mixture of phenyl 5-{[2-(2-chloro-4-fluorophenyl)-1-oxo-2-azaspiro[4.5]dec-8-yl]carbamoyl}-1H-imidazole-4-carboxylate (isomer 2) (100 mg, 196 μmol) and ammonia in dioxane (780 μl, 0.50 M, 390 μmol) in THF (1.9 ml) was added triethylamine (82 μl, 590 μmol) and the reaction was stirred at room temperature for 18 hours. Ammonia in dioxane (3 ml, 0.50 M, 1.5 mmol) was added and the mixture was stirred for 3 hours at room temperature. For work up the reaction was concentrated and the residue was purified by HPLC (Method 8) to give the title product (42 mg)

LC-MS (Method 4): Rₜ=0.96 min; MS (ESIpos): m/z=434 [M+H]⁺

¹H-NMR (400 MHz, DMSO-d6) δ[ppm]: 1.233 (0.70), 1.458 (2.69), 1.477 (2.49), 1.492 (3.42), 1.543 (0.84), 1.718 (1.74), 1.742 (3.73), 1.751 (3.94), 1.775 (3.28), 1.857 (3.94), 1.880 (2.94), 1.891 (2.60), 1.909 (4.41), 1.943 (4.48), 1.969 (2.29), 2.052 (1.38), 2.071 (5.70), 2.074 (5.18), 2.088 (8.64), 2.106 (4.19), 2.327 (1.38), 2.332 (1.11), 2.522 (4.37), 2.539 (1.43), 2.669 (1.40), 2.673 (1.13), 3.587 (4.73), 3.604 (9.57), 3.613 (4.39), 3.620 (5.34), 3.973 (2.31), 7.277 (1.88), 7.284 (2.51), 7.299 (3.58), 7.306 (4.55), 7.319 (2.51), 7.326 (3.10), 7.456 (4.21), 7.470 (5.00), 7.478 (4.01), 7.487 (1.70), 7.492 (4.03), 7.508 (1.04), 7.566 (4.28), 7.573 (4.89), 7.587 (4.41), 7.594 (4.73), 7.754 (0.97), 7.782 (16.00), 7.811 (7.81), 7.939 (3.80), 8.047 (1.09), 8.067 (1.04), 10.314 (0.91), 11.448 (3.15), 11.465 (3.10), 13.174 (2.53).

### Example 343

### N-[(trans)-2-(2-chloro-4-fluorophenyl)-1-oxo-2-azaspiro[4.5]dec-8-yl]-1-methyl-1H-pyrrolo[3,2-b]pyridine-3-carboxamide

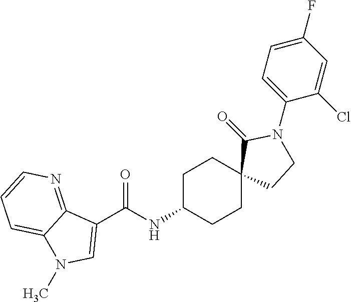 [SMILES: Cn1cc(C(=O)N[C@H]2CC[C@@]3(CCN(c4ccc(F)cc4Cl)C3=O)CC2)c2ncccc21]

To a mixture of N-[2-(2-chloro-4-fluorophenyl)-1-oxo-2-azaspiro[4.5]dec-8-yl]-1H-pyrrolo[3,2-b]pyridine-3-carboxamide (isomer 1) (50.0 mg, 113 μmol) and potassium carbonate (78.4 mg, 567 μmol) in DMF (1.0 ml) was added iodomethane (14 μl, 230 μmol) and the mixture was stirred at 65° C. for 4 hours and for 3 days at room temperature. For work-up the mixture was poured into ammonium chloride solution and the mixture was extracted with dichloromethane/2-propanol (4:1). The combined organic phases were washed with brine and the organic phase was filtrated over a hydrophobic filter and concentrated to give the title product (47 mg).

LC-MS (Method 2): Rₜ=1.20 min; MS (ESIneg): m/z=455 [M+H]⁻

¹H-NMR (400 MHz, DMSO-d6) b [ppm]: 1.450 (0.59), 1.472 (0.93), 1.491 (0.77), 1.522 (0.44), 1.677 (3.04), 1.686 (3.45), 1.702 (2.24), 1.990 (1.08), 1.998 (1.13), 2.010 (0.72), 2.022 (1.05), 2.030 (0.98), 2.161 (1.90), 2.178 (3.60), 2.195 (2.01), 2.326 (0.41), 2.518 (1.13), 2.522 (0.77), 2.668 (0.41), 2.727 (0.51), 2.888 (0.62), 3.610 (2.06), 3.617 (0.54), 3.627 (3.55), 3.644 (1.98), 3.822 (0.46), 3.832 (0.57), 3.841 (0.46), 3.851 (0.57), 3.860 (0.49), 3.885 (16.00), 7.286 (0.95), 7.291 (2.14), 7.294 (1.18), 7.303 (2.06), 7.308 (1.62), 7.312 (2.37), 7.315 (1.67), 7.324 (2.14), 7.329 (1.05), 7.336 (1.16), 7.468 (1.90), 7.483 (2.03), 7.490 (1.65), 7.505 (1.52), 7.577 (1.93), 7.584 (1.98), 7.599 (1.98), 7.605 (1.88), 8.034 (2.34), 8.037 (2.32), 8.055 (2.24), 8.058 (2.16), 8.210 (6.77), 8.500 (2.16), 8.503 (2.32), 8.512 (2.29), 8.515 (2.01), 8.633 (1.59), 8.653 (1.54).

### Example 344

### N-[(trans)-2-(2-chloro-4-fluorophenyl)-1-oxo-2-azaspiro[4.5]dec-8-yl]-1-(2-hydroxyethyl)-1H-pyrrolo[3,2-b]pyridine-3-carboxamide

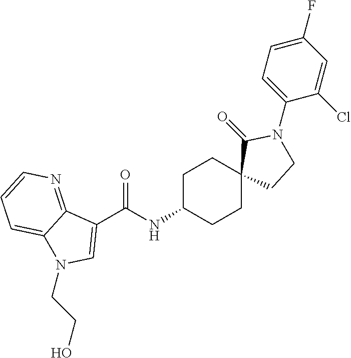 [SMILES: O=C(N[C@H]1CC[C@@]2(CCN(c3ccc(F)cc3Cl)C2=O)CC1)c1cn(CCO)c2cccnc12]

To a mixture of N-[2-(2-chloro-4-fluorophenyl)-1-oxo-2-azaspiro[4.5]dec-8-yl]-1H-pyrrolo[3,2-b]pyridine-3-carboxamide (isomer 1) (70.0 mg, 159 μmol) and potassium carbonate (110 mg, 794 μmol) in DMF (860 μl) was added 2-bromoethanol (23 μl, 320 μmol) and the mixture was stirred at 65° C. for 4 hours and for 18 hours at room temperature. For work-up the mixture was poured into ammonium chloride solution and the mixture was extracted with dichloromethane/2-propanol (4:1). The combined organic phases were washed with brine and the organic phase was filtrated over a hydrophobic filter and concentrated. The crude product was purified by preparative HPLC to give the title compound (6.5 mg).

LC-MS (Method 2): Rₜ=1.12 min; MS (ESIpos): m/z=485 [M+H]⁺

¹H-NMR (400 MHz, DMSO-d6) δ[ppm]: 1.107 (2.98), 1.230 (1.19), 1.348 (0.72), 1.452 (1.33), 1.475 (2.01), 1.494 (1.73), 1.525 (0.93), 1.681 (6.54), 1.691 (7.37), 1.706 (4.85), 1.713 (4.31), 1.990 (2.37), 2.000 (2.44), 2.023 (2.30), 2.031 (2.12), 2.164 (4.10), 2.181 (7.80), 2.198 (4.35), 2.332 (1.51), 2.518 (6.65), 2.522 (4.31), 2.673 (1.51), 3.504 (1.01), 3.510 (0.86), 3.612 (4.42), 3.620 (1.22), 3.630 (7.69), 3.646 (4.24), 3.703 (1.80), 3.716 (4.71), 3.729 (4.82), 3.742 (1.91), 3.829 (0.97), 3.838 (1.19), 3.848 (0.97), 3.858 (1.19), 3.868 (0.90), 4.303 (3.74), 4.317 (6.36), 4.330 (3.49), 4.919 (2.09), 4.932 (4.57), 4.945 (1.98), 7.265 (4.28), 7.277 (3.96), 7.286 (4.60), 7.288 (3.13), 7.295 (3.13), 7.297 (4.67), 7.310 (3.06), 7.317 (3.34), 7.331 (2.27), 7.338 (2.66), 7.470 (4.21), 7.485 (4.42), 7.492 (3.60), 7.507 (3.31), 7.579 (4.31), 7.586 (4.39), 7.601 (4.39), 7.608 (4.17), 8.069 (4.85), 8.072 (5.00), 8.090 (4.64), 8.093 (4.64), 8.197 (16.00), 8.482 (4.93), 8.485 (5.07), 8.494 (4.93), 8.497 (4.46), 8.666 (3.60), 8.686 (3.49).

### Example 345

### N-[(trans)-2-(2-chloro-4-fluorophenyl)-1-oxo-2-azaspiro[4.5]dec-8-yl]-1-(2-methoxyethyl)-1H-pyrrolo[3,2-b]pyridine-3-carboxamide

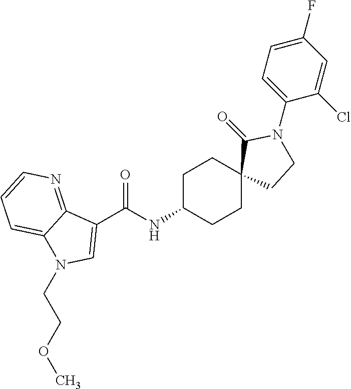 [SMILES: COCCn1cc(C(=O)N[C@H]2CC[C@@]3(CCN(c4ccc(F)cc4Cl)C3=O)CC2)c2ncccc21]

To a mixture of N-[2-(2-chloro-4-fluorophenyl)-1-oxo-2-azaspiro[4.5]dec-8-yl]-1H-pyrrolo[3,2-b]pyridine-3-carboxamide (isomer 1) (90.0 mg, 204 μmol) and potassium carbonate (141 mg, 1.02 mmol) in DMF (1.1 ml) was added 2-bromoethyl methyl ether (52 μl, 410 μmol) and the mixture was stirred at 65° C. for 4 hours and for 16 hours at room temperature. For work-up the mixture was poured into ammonium chloride solution and the mixture was extracted with dichloromethane/2-propanol (4:1). The combined organic phases were washed with brine and the organic phase was filtrated over a hydrophobic filter and concentrated. The crude product was purified by HPLC (Method 9) to give the title compound (45 mg).

LC-MS (Method 2): Rₜ=1.23 min; MS (ESIneg): m/z=497 [M−H]⁻

¹H-NMR (400 MHz, DMSO-d6) δ[ppm]: 1.681 (1.90), 1.690 (2.10), 1.705 (1.40), 1.712 (1.26), 2.163 (1.18), 2.181 (2.20), 2.198 (1.21), 3.205 (16.00), 3.612 (1.27), 3.630 (2.21), 3.647 (1.32), 3.654 (1.32), 3.667 (2.07), 3.680 (1.28), 4.436 (1.18), 4.449 (1.91), 4.461 (1.09), 7.275 (1.15), 7.287 (1.39), 7.296 (1.61), 7.308 (1.70), 7.317 (0.94), 7.338 (0.68), 7.470 (1.14), 7.485 (1.21), 7.492 (0.98), 7.507 (0.92), 7.580 (1.17), 7.587 (1.21), 7.601 (1.21), 7.608 (1.15), 8.089 (1.31), 8.092 (1.32), 8.110 (1.24), 8.113 (1.21), 8.201 (4.45), 8.491 (1.33), 8.494 (1.37), 8.502 (1.30), 8.505 (1.15), 8.663 (1.00), 8.682 (0.97).

### Example 346

### N-[(trans)-1-oxo-2-(pyridin-2-yl)-2-azaspiro[4.5]dec-8-yl]pyrazolo[1,5-a]pyrimidine-3-carboxamide

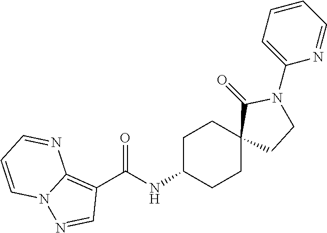 [SMILES: O=C(N[C@H]1CC[C@@]2(CCN(c3ccccn3)C2=O)CC1)c1cnn2cccnc12]

To a solution of pyrazolo[1,5-a]pyrimidine-3-carboxylic acid (62.3 mg, 382 μmol) and 8-amino-2-(pyridin-2-yl)-2-azaspiro[4.5]decan-1-one (isomer 1) (75.0 mg, 306 μmol) in DMF (3.4 ml) was added PyBOP (175 mg, 336 μmol) and N,N-diisopropylethylamine (270 μl, 1.5 mmol) and the reaction was stirred for 12 hours at room temperature. For work-up the mixture was concentrated and the crude product was purified by HPLC (Method 9) to give the title product (26 mg).

LC-MS (Method 2): Rₜ=0.93 min; MS (ESIpos): m/z=391 [M+H]⁺

¹H-NMR (400 MHz, DMSO-d6) δ[ppm]: 1.463 (2.10), 1.474 (2.18), 1.493 (2.61), 1.505 (2.39), 1.524 (1.17), 1.536 (1.02), 1.658 (1.17), 1.681 (7.75), 1.709 (2.61), 1.717 (2.97), 1.947 (2.57), 1.956 (2.70), 1.968 (1.64), 1.980 (2.50), 1.988 (2.27), 2.073 (4.48), 2.090 (6.96), 2.108 (4.66), 2.323 (0.77), 2.327 (1.06), 2.331 (0.76), 2.518 (3.58), 2.523 (2.30), 2.665 (0.76), 2.669 (1.06), 3.835 (1.04), 3.845 (1.29), 3.854 (1.06), 3.864 (1.28), 3.874 (1.01), 3.941 (4.84), 3.953 (2.03), 3.959 (7.28), 3.964 (2.27), 3.976 (4.71), 7.131 (2.53), 7.133 (2.62), 7.143 (2.48), 7.145 (2.86), 7.149 (2.89), 7.151 (2.66), 7.161 (2.68), 7.163 (2.80), 7.260 (4.22), 7.271 (4.03), 7.277 (4.04), 7.288 (4.30), 7.795 (2.30), 7.800 (2.41), 7.814 (2.93), 7.819 (5.07), 7.821 (6.20), 7.835 (3.18), 7.840 (5.34), 8.323 (2.80), 8.325 (5.54), 8.328 (3.43), 8.344 (2.84), 8.347 (4.89), 8.349 (3.18), 8.386 (2.73), 8.389 (3.07), 8.391 (3.34), 8.394 (2.77), 8.399 (2.89), 8.401 (3.15), 8.403 (3.04), 8.406 (2.64), 8.581 (16.00), 8.821 (4.67), 8.824 (5.07), 8.831 (4.73), 8.835 (4.58), 9.308 (4.69), 9.312 (4.87), 9.326 (4.84), 9.330 (4.55).

### Example 347

### N-[(trans)-2-(2-chloro-4-fluorophenyl)-1-oxo-2-azaspiro[4.5]dec-8-yl][1,2,4]triazolo[4,3-b]pyridazine-3-carboxamide

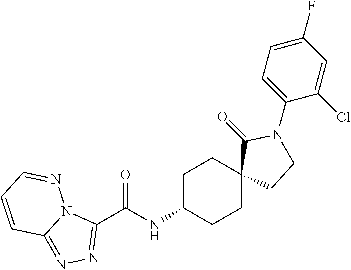 [SMILES: O=C(N[C@H]1CC[C@@]2(CCN(c3ccc(F)cc3Cl)C2=O)CC1)c1nnc2cccnn12]

To a solution of 8-amino-2-(2-chloro-4-fluorophenyl)-2-azaspiro[4.5]decan-1-one (isomer 1) (100 mg, 337 μmol) and potassium [1,2,4]triazolo[4,3-b]pyridazine-3-carboxylate (81.8 mg, 404 μmol) in DMF (3.5 ml) was added PyBOP (193 mg, 371 μmol) and N,N-diisopropylethylamine (290 μl, 1.7 mmol) and the reaction was stirred for 12 hours at room temperature. For work-up the mixture was concentrated and ethanol was added to the residue. The resulting precipitate was filtrated and the precipitate was washed with water and ethanol to give the title product (99 mg).

LC-MS (Method 2): Rₜ=0.92 min; MS (ESIneg): m/z=441 [M−H]⁻

¹H-NMR (400 MHz, DMSO-d6) δ[ppm]: 1.035 (0.68), 1.052 (1.48), 1.070 (0.80), 1.542 (0.97), 1.558 (1.54), 1.572 (2.33), 1.585 (3.30), 1.614 (4.67), 1.639 (5.98), 1.665 (9.22), 1.674 (10.48), 1.682 (10.02), 1.699 (10.70), 1.890 (4.61), 1.904 (4.33), 1.921 (4.73), 1.930 (4.44), 2.114 (8.65), 2.121 (2.16), 2.131 (16.00), 2.149 (8.88), 2.318 (1.08), 2.323 (2.51), 2.327 (3.47), 2.331 (2.45), 2.337 (1.08), 2.518 (10.59), 2.523 (7.12), 2.660 (1.14), 2.665 (2.56), 2.669 (3.47), 2.674 (2.45), 2.678 (1.08), 3.613 (9.34), 3.620 (2.39), 3.630 (15.89), 3.639 (2.28), 3.647 (8.77), 3.848 (0.91), 3.876 (2.11), 3.886 (1.88), 3.896 (1.94), 3.907 (1.65), 3.924 (0.85), 3.998 (0.57), 5.759 (3.42), 7.289 (3.70), 7.296 (4.33), 7.311 (6.04), 7.318 (6.55), 7.332 (4.61), 7.339 (5.24), 7.463 (8.60), 7.470 (12.36), 7.478 (9.62), 7.481 (11.84), 7.485 (7.69), 7.493 (10.99), 7.499 (7.12), 7.504 (12.58), 7.580 (8.37), 7.587 (8.65), 7.601 (8.65), 7.608 (8.37), 8.483 (11.56), 8.487 (12.24), 8.507 (10.76), 8.510 (10.82), 8.779 (9.96), 8.783 (10.19), 8.790 (9.62), 8.793 (9.17), 8.914 (6.78), 8.934 (6.72).

### Example 348

### N-[(trans)-2-(2-chloro-4-fluorophenyl)-1-oxo-2-azaspiro[4.5]dec-8-yl]-5,7-diethyl pyrazolo[1,5-a]pyrimidine-3-carboxamide

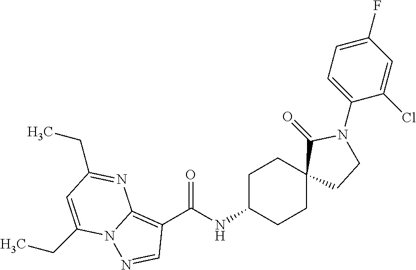 [SMILES: CCc1cc(CC)n2ncc(C(=O)N[C@H]3CC[C@@]4(CCN(c5ccc(F)cc5Cl)C4=O)CC3)c2n1]

To a solution of 8-amino-2-(2-chloro-4-fluorophenyl)-2-azaspiro[4.5]decan-1-one (isomer 1) (100 mg, 337 μmol) and 5,7-diethylpyrazolo[1,5-a]pyrimidine-3-carboxylic acid (94.2 mg, 98% purity, 421 μmol) in DMF (1.0 ml) was added PyBOP (210 mg, 404 μmol) and N,N-diisopropylethylamine (290 μl, 1.7 mmol) and the reaction was stirred for 12 hours at room temperature. For work-up the reaction was poured into water and the resulting precipitate was filtrated to give after trituration with 2-propanol the title compound (18 mg).

LC-MS (Method 2): Rₜ=1.33 min; MS (ESIneg): m/z=496 [M−H]⁻

¹H-NMR (400 MHz, DMSO-d6) δ[ppm]: 1.331 (6.72), 1.341 (7.02), 1.349 (16.00), 1.360 (15.25), 1.368 (7.14), 1.379 (6.72), 1.443 (1.14), 1.686 (3.84), 1.694 (4.44), 1.712 (2.70), 1.720 (1.92), 2.030 (1.38), 2.039 (1.44), 2.062 (1.35), 2.072 (1.23), 2.137 (2.43), 2.154 (4.65), 2.171 (2.61), 2.327 (0.84), 2.518 (2.55), 2.523 (1.77), 2.669 (0.84), 2.943 (1.89), 2.962 (5.91), 2.980 (5.85), 2.998 (1.71), 3.128 (1.23), 3.145 (3.63), 3.147 (3.63), 3.164 (3.63), 3.184 (1.14), 3.613 (2.61), 3.630 (4.59), 3.647 (2.52), 7.122 (5.58), 7.287 (1.11), 7.295 (1.20), 7.310 (1.74), 7.317 (1.92), 7.330 (1.29), 7.338 (1.47), 7.466 (2.37), 7.480 (2.52), 7.488 (2.07), 7.502 (1.92), 7.577 (2.46), 7.585 (2.55), 7.599 (2.49), 7.606 (2.49), 8.143 (2.07), 8.161 (1.98), 8.501 (11.38).

### Example 349

### 6-chloro-N-[(trans)-2-(2-chloro-4-fluorophenyl)-1-oxo-2-azaspiro[4.5]dec-8-yl]-8-[(3,3,3-trifluoropropyl)amino]imidazo[1,2-b]pyridazine-3-carboxamide

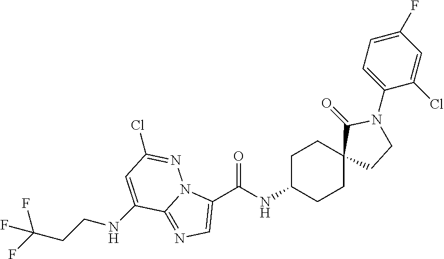 [SMILES: O=C(N[C@H]1CC[C@@]2(CCN(c3ccc(F)cc3Cl)C2=O)CC1)c1cnc2c(NCCC(F)(F)F)cc(Cl)nn12]

To a solution of 8-amino-2-(2-chloro-4-fluorophenyl)-2-azaspiro[4.5]decan-1-one (isomer 1) (63.8 mg, 215 μmol) and 6-chloro-8-[(3,3,3-trifluoropropyl)amino]imidazo[1,2-b]pyridazine-3-carboxylic acid (83.0 mg, 269 μmol) in DMF (2.4 ml, 31 mmol) was added PyBOP (134 mg, 0.258 mmol) and N,N-diisopropylethylamine (190 μl, 1.1 mmol) and the reaction was stirred for 12 hours at room temperature. For work-up the reaction mixture was concentrated under reduced pressure and the residue was purified by HPLC [Instrument: Labomatic HD5000, Labocord-5000; Gilson GX-241, Labcol Vario 4000, column: XBrigde C18 5μ 150×30 mm; eluent A: water+0.1 Vol-% formic acid (99%); eluent C: acetonitrile; gradient: 0-7 min 25-95% C; flow 50.0 ml/min; UV @ 254 nm] to give after trituration with methanol the title product (7.8 mg).

LC-MS (Method 2): Rₜ=1.37 min; MS (ESIneg): m/z=585 [M−H]⁻

¹H-NMR (400 MHz, DMSO-d6) δ[ppm]: 1.107 (0.62), 1.456 (1.32), 1.481 (1.86), 1.496 (1.63), 1.526 (0.93), 1.683 (6.21), 1.693 (7.15), 1.707 (4.50), 2.011 (2.25), 2.020 (2.41), 2.042 (2.25), 2.052 (2.02), 2.084 (9.71), 2.139 (4.04), 2.155 (7.69), 2.172 (4.27), 2.318 (1.48), 2.322 (3.26), 2.327 (4.66), 2.332 (3.34), 2.336 (1.55), 2.518 (15.22), 2.523 (9.63), 2.660 (2.41), 2.664 (3.65), 2.669 (6.37), 2.673 (4.27), 2.678 (2.17), 2.687 (3.50), 2.698 (1.86), 2.704 (2.02), 2.715 (3.26), 2.732 (1.79), 2.744 (1.09), 3.159 (6.76), 3.171 (7.69), 3.611 (5.59), 3.629 (9.09), 3.646 (5.20), 3.824 (1.01), 3.833 (1.17), 3.852 (1.17), 4.083 (0.54), 4.096 (1.79), 4.109 (1.63), 6.570 (5.05), 7.288 (1.86), 7.295 (2.17), 7.309 (2.95), 7.317 (3.11), 7.330 (2.17), 7.337 (2.49), 7.466 (3.96), 7.481 (4.19), 7.489 (3.50), 7.503 (3.18), 7.578 (4.19), 7.585 (4.35), 7.599 (4.19), 7.606 (4.12), 8.045 (16.00), 8.312 (2.56), 8.385 (2.25), 8.404 (2.10).

### Example 350

### N-[(trans)-2-(2-chloro-4-fluorophenyl)-1-oxo-2-azaspiro[4.5]dec-8-yl]-5H-pyrrolo[2,3-b]pyrazine-7-carboxamide

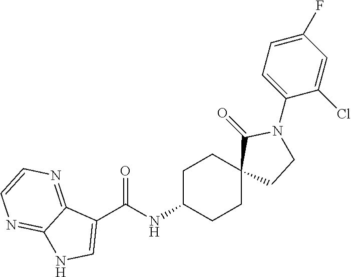 [SMILES: O=C(N[C@H]1CC[C@@]2(CCN(c3ccc(F)cc3Cl)C2=O)CC1)c1c[nH]c2nccnc12]

To a solution of 8-amino-2-(2-chloro-4-fluorophenyl)-2-azaspiro[4.5]decan-1-one (isomer 1) (100 mg, 337 μmol) and 5H-pyrrolo[2,3-b]pyrazine-7-carboxylic acid (68.7 mg, 421 μmol) in DMF (3.5 ml) was added PyBOP (210 mg, 404 μmol) and N,N-diisopropylethylamine (290 μl, 1.7 mmol) and the reaction was stirred for 12 hours at room temperature. For work-up the reaction was concentrated under reduced pressure and the crude product was purified by HPLC to give the title product (6.8 mg).

LC-MS (Method 2): Rₜ=1.02 min; MS (ESIpos): m/z=442 [M+H]⁺

¹H-NMR (400 MHz, DMSO-d6) δ[ppm]: 1.470 (1.33), 1.493 (2.08), 1.512 (1.79), 1.543 (0.87), 1.678 (6.82), 1.687 (7.51), 1.703 (4.91), 1.880 (1.04), 1.983 (2.43), 1.991 (2.54), 2.015 (2.37), 2.022 (2.14), 2.161 (4.10), 2.178 (7.74), 2.195 (4.27), 2.318 (1.21), 2.323 (2.48), 2.327 (3.47), 2.331 (2.43), 2.337 (1.10), 2.518 (12.71), 2.523 (8.14), 2.660 (1.16), 2.665 (2.54), 2.669 (3.47), 2.674 (2.43), 2.678 (1.16), 3.611 (4.45), 3.628 (7.68), 3.638 (1.21), 3.645 (4.22), 3.839 (0.98), 3.849 (1.21), 3.859 (0.98), 3.868 (1.21), 3.878 (0.87), 7.288 (1.73), 7.296 (2.02), 7.310 (2.89), 7.317 (3.12), 7.331 (2.19), 7.338 (2.37), 7.469 (3.87), 7.483 (4.16), 7.491 (3.41), 7.505 (3.18), 7.580 (4.04), 7.587 (4.10), 7.601 (4.10), 7.608 (3.93), 8.098 (3.52), 8.118 (3.41), 8.384 (6.12), 8.391 (6.47), 8.410 (16.00), 8.539 (6.41), 8.545 (5.60).

### Example 351

### methyl 3-({trans-4-[(2-chloro-4,6-difluorophenyl)carbamoyl]cyclohexyl}carbamoyl)-5-methylpyrazolo[1,5-a]pyrimidine-7-carboxylate

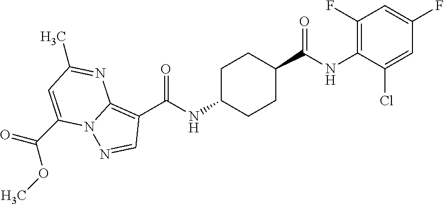 [SMILES: COC(=O)c1cc(C)nc2c(C(=O)N[C@H]3CC[C@H](C(=O)Nc4c(F)cc(F)cc4Cl)CC3)cnn12]

To a solution of trans-4-amino-N-(2-chloro-4,6-difluorophenyl)cyclohexanecarboxamide hydrochloric salt (74.7 mg, 230 μmol) in DMF (1.9 ml) was added PYBOP (159 mg, 306 μmol) and N,N-diisopropylethylamine (180 μl, 1.0 mmol). 7-(methoxycarbonyl)-5-methylpyrazolo[1,5-a]pyrimidine-3-carboxylic acid (60.0 mg, 255 μmol) was added and the reaction was stirred for 16 hours at room temperature. For work-up the mixture was poured into water and the resulting precipitate was filtrated. The residue was washed with water and the crude product was purified by HPLC to give the title product (18 mg).

LC-MS (Method 1): Rₜ=1.05 min; MS (ESIpos): m/z=506 [M+H]⁺

¹H-NMR (400 MHz, DMSO-d6) δ[ppm]: 1.106 (0.85), 1.381 (1.44), 1.396 (1.02), 1.420 (1.10), 1.954 (1.19), 2.518 (4.89), 2.523 (3.70), 2.716 (15.46), 4.013 (16.00), 7.400 (0.73), 7.416 (0.93), 7.424 (1.30), 7.447 (1.70), 7.451 (1.10), 7.469 (0.96), 7.472 (1.02), 7.600 (6.19), 7.898 (1.47), 7.918 (1.41), 8.574 (6.25), 9.600 (2.60).

### Example 352

### methyl 3-({trans-4-[(2-chloro-4-fluorophenyl)carbamoyl]cyclohexyl}carbamoyl)-5-methylpyrazolo[1,5-a]pyrimidine-7-carboxylate

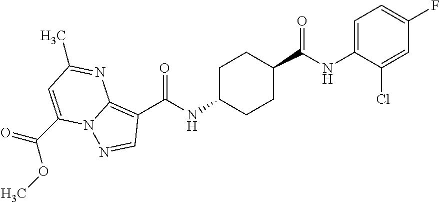 [SMILES: COC(=O)c1cc(C)nc2c(C(=O)N[C@H]3CC[C@H](C(=O)Nc4ccc(F)cc4Cl)CC3)cnn12]

To a solution of trans-4-amino-N-(2-chloro-4-fluorophenyl)cyclohexanecarboxamide hydrochloride (70.5 mg, 230 μmol) in DMF (1.9 ml) was added PyBOP (159 mg, 306 μmol) and N,N-diisopropylethylamine (180 μl, 1.0 mmol). 7-(methoxycarbonyl)-5-methylpyrazolo[1,5-a]pyrimidine-3-carboxylic acid (60.0 mg, 255 μmol) was added and the reaction was stirred for 16 hours at room temperature. For work-up the mixture was poured into water and the resulting precipitate was collected by filtration, washed with water and dried to give the title compound (71 mg).

LC-MS (Method 1): Rₜ=1.12 min; MS (ESIpos): m/z=488 [M+H]⁺

¹H-NMR (400 MHz, DMSO-d6) δ[ppm]: 1.404 (1.13), 1.565 (1.13), 1.603 (1.13), 1.938 (1.35), 1.968 (1.13), 2.060 (1.13), 2.083 (1.13), 2.523 (3.15), 2.710 (16.00), 3.508 (2.70), 3.987 (0.90), 4.008 (15.32), 7.188 (0.68), 7.195 (0.68), 7.210 (1.13), 7.217 (1.35), 7.231 (0.90), 7.238 (0.90), 7.478 (1.35), 7.486 (1.35), 7.500 (1.35), 7.507 (1.35), 7.578 (1.35), 7.591 (7.44), 7.600 (1.58), 7.614 (1.35), 7.909 (1.35), 7.929 (1.35), 8.569 (6.54), 9.510 (2.70).

### Example 353

### N-[(trans)-2-(2-chloro-5-methoxyphenyl)-1-oxo-2-azaspiro[4.5]dec-8-yl]pyrazolo[1,5-a]pyrimidine-3-carboxamide

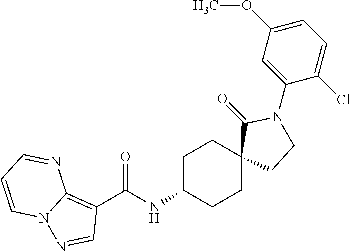 [SMILES: COc1ccc(Cl)c(N2CC[C@]3(CC[C@@H](NC(=O)c4cnn5cccnc45)CC3)C2=O)c1]

To a solution of 8-amino-2-(2-chloro-5-methoxyphenyl)-2-azaspiro[4.5]decan-1-one (isomer 1) (42.0 mg, 136 μmol) and pyrazolo[1,5-a]pyrimidine-3-carboxylic acid (27.7 mg, 170 μmol) in DMF (1.5 ml) was added PyBOP (77.9 mg, 150 μmol) and N,N-diisopropylethylamine (120 μl, 680 μmol). The reaction was stirred for 12 hours at room temperature. For work-up the mixture was concentrated under reduced pressure and the residue was purified by HPLC [Instrument: Waters Autopurificationsystem; column: YMC Triart C18 5μ 100×30 mm; eluent A: water+0.1 Vol-% formic acid (99%), eluent B: acetonitrile; gradient: 0.00-0.50 min 20% B (25->70 mL/min), 0.51-5.50 min 36-56% B (70 mL/min), DAD scan: 210-400 nm] to give the title product (42 mg).

LC-MS (Method 2): Rₜ=1.05 min; MS (ESIpos): m/z=454 [M+H]⁺

1H-NMR (400 MHz, DMSO-d6): δ [ppm]=9.32 (dd, 1H), 8.83 (dd, 1H), 8.68-6.77 (m, 6H), 3.91-3.80 (m, 1H), 3.77 (s, 3H), 3.64 (t, 2H), 2.16 (t, 2H), 2.01-1.87 (m, 2H), 1.79-1.62 (m, 4H), 1.49 (br s, 2H).

### Example 354

### N-{(trans)-2-[2-chloro-4-(trifluoromethoxy)phenyl]-1-oxo-2-azaspiro[4.5]dec-8-yl}pyrazolo[1,5-a]pyrimidine-3-carboxamide

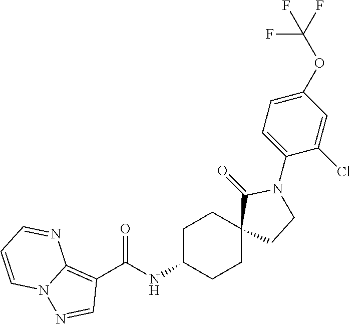 [SMILES: O=C(N[C@H]1CC[C@@]2(CCN(c3ccc(OC(F)(F)F)cc3Cl)C2=O)CC1)c1cnn2cccnc12]

To a solution of 8-amino-2-[2-chloro-4-(trifluoromethoxy)phenyl]-2-azaspiro[4.5]decan-1-one (isomer 1) (131 mg, 361 μmol) and pyrazolo[1,5-a]pyrimidine-3-carboxylic acid (73.6 mg, 451 μmol) in DMF (4.3 ml) was added PyBOP (225 mg, 433 μmol) and N,N-diisopropylethylamine (310 μl, 1.8 mmol). The reaction was stirred for 3 d at room temperature. For work-up the reaction was poured into ice water and the resulting precipitate collected by filtration, washed with water and methanol to give after drying the title product (43 mg).

LC-MS (Method 2): Rₜ=1.19 min; MS (ESIpos): m/z=508 [M+H]⁺

1H-NMR (400 MHz, DMSO-d6): δ [ppm]=9.32 (dd, 1H), 8.83 (dd, 1H), 8.58 (s, 1H), 7.83 (d, 1H), 7.73 (d, 1H), 7.59 (d, 1H), 7.51-7.43 (m, 1H), 7.35-7.18 (m, 1H), 3.95-3.79 (m, 1H), 3.67 (t, 2H), 2.18 (t, 2H), 2.05-1.90 (m, 2H), 1.69 (dd, 4H), 1.59-1.42 (m, 2H).

### Example 355

### N-[(trans)-2-(2-chloro-4-fluorophenyl)-3-methyl-1-oxo-2-azaspiro[4.5]dec-8-yl]pyrazolo[1,5-a]pyrimidine-3-carboxamide (mixture of isomers at C3)

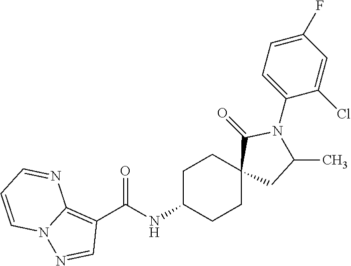

To a solution of 8-amino-2-(2-chloro-4-fluorophenyl)-3-methyl-2-azaspiro[4.5]decan-1-one (trans isomer, mixture of isomers at C3) (31.0 mg, 99.7 μmol) and pyrazolo[1,5-a]pyrimidine-3-carboxylic acid (20.3 mg, 125 μmol) in DMF (1.2 ml) was added PyBOP (62.3 mg, 120 μmol) and N,N-diisopropylethylamine (87 μl, 500 μmol). The reaction was stirred for 16 hours at room temperature. For work-up the reaction was concentrated under reduced pressure and the residue was purified by flash chromatography (hexane/ethyl acetate-gradient, 50%->100% ethyl acetate, ethyl acetate/methanol-gradient, 0->100% ethanol) followed by HPLC purification to give the title compound (12 mg).

LC-MS (Method 2): Rₜ=1.11 min; MS (ESIpos): m/z=456 [M+H]⁺

The title compound (12 mg) was separated into enantiomers by chiral HPLC to give enantiomer 1 (Rₜ 10.9-11.9 min) and enantiomer 2 (Rₜ 11.9-14.1 min).

### Preparative Chiral HPLC Method:

Instrument: Labomatic HD5000, Labocord-5000; Gilson GX-241, Labcol Vario 4000, column: Chiralpak IB 5μ 250×30 mm; eluent A: hexane; eluent B: ethanol; isocratic: 70% A+30% B+0.1 Vol-% diethyl amine (99%); flow 50.0 ml/min; UV 254 nm

### Analytical Chiral HPLC Method:

Instrument: Agilent HPLC 1260; column: Chiralpak IB 3p 100×4.6 mm; eluent A: Hexane+0.1 Vol-% diethyl amine (99%); eluent B: ethanol; isocratic: 70% A+30% B; flow 1.4 ml/min; temperature: 25° C.; DAD 254 nm

### Example 356

### N-[(trans)-2-(2-chloro-4-fluorophenyl)-3-methyl-1-oxo-2-azaspiro[4.5]dec-8-yl]pyrazolo[1,5-a]pyrimidine-3-carboxamide (enantiomer 1)

Chiral separation (method see example 355 Rₜ 10.9-11.9 min) gave the title product (5 mg) in a purity of 86%.

Analytical chiral HPLC (method see example 355): Rₜ=4.18 min, ee=>99.9%

¹H-NMR (400 MHz, DMSO-d6) δ[ppm]: 1.041 (3.93), 1.057 (3.97), 1.070 (0.50), 1.084 (0.62), 1.124 (7.33), 1.142 (16.00), 1.160 (7.83), 1.232 (1.43), 1.259 (1.08), 1.403 (0.46), 1.434 (0.46), 1.535 (1.23), 1.556 (1.77), 1.566 (1.73), 1.589 (1.20), 1.775 (0.58), 1.799 (0.46), 1.831 (0.50), 1.856 (0.62), 1.865 (0.69), 1.906 (1.04), 1.959 (0.73), 1.981 (1.04), 2.337 (0.69), 2.518 (8.37), 2.523 (6.05), 2.537 (0.66), 2.546 (0.89), 2.564 (0.73), 2.579 (0.62), 2.596 (0.54), 2.679 (0.69), 2.868 (1.62), 2.886 (4.86), 2.904 (4.74), 2.922 (1.50), 7.263 (1.70), 7.274 (1.58), 7.281 (1.58), 7.291 (2.31), 7.298 (0.69), 7.312 (1.04), 7.319 (1.16), 7.333 (0.81), 7.340 (0.89), 7.585 (1.16), 7.592 (1.16), 7.607 (1.16), 7.615 (1.08), 7.818 (1.31), 7.838 (1.27), 8.577 (7.06), 8.819 (1.93), 8.823 (2.20), 8.829 (2.00), 8.834 (1.89), 9.311 (2.08), 9.315 (2.20), 9.328 (2.20), 9.333 (1.93).

### Example 357

### N-[(trans)-2-(2-chloro-4-fluorophenyl)-3-methyl-1-oxo-2-azaspiro[4.5]dec-8-yl]pyrazolo[1,5-a]pyrimidine-3-carboxamide (enantiomer 2)

Chiral separation (method see example 355 Rₜ 11.9-14.1 min) gave the title product (6 mg) in a purity of 60%.

Analytical chiral HPLC (method see example 355): Rₜ=5.46 min, ee=>99.9%

¹H-NMR (400 MHz, CHLOROFORM-d) δ [ppm]: 1.133 (3.89), 1.149 (3.81), 1.260 (1.19), 1.317 (7.51), 1.335 (16.00), 1.353 (7.81), 1.475 (0.41), 1.660 (0.56), 1.851 (1.14), 1.860 (1.25), 1.884 (2.17), 2.170 (0.42), 2.179 (0.99), 2.206 (0.82), 2.215 (0.57), 2.555 (0.56), 2.572 (0.62), 2.587 (0.56), 2.604 (0.53), 2.993 (1.42), 3.011 (2.17), 3.024 (2.14), 3.041 (1.34), 4.109 (0.47), 4.126 (0.40), 6.993 (1.01), 7.003 (1.01), 7.006 (0.41), 7.010 (0.99), 7.017 (0.63), 7.020 (1.10), 7.024 (0.62), 7.037 (0.58), 7.039 (0.71), 7.044 (0.66), 7.046 (0.82), 7.058 (0.65), 7.065 (0.69), 7.166 (0.74), 7.179 (0.78), 7.187 (0.59), 7.202 (0.55), 7.219 (0.96), 7.226 (0.93), 7.239 (0.92), 7.246 (0.87), 7.785 (0.47), 7.804 (0.46), 8.637 (1.23), 8.641 (1.40), 8.647 (1.14), 8.651 (1.16), 8.702 (3.65), 8.785 (1.26), 8.790 (1.28), 8.803 (1.32), 8.808 (1.17).

### Example 358

### N-[(trans)-2-(2-chloro-4-fluorophenyl)-1-oxo-2-azaspiro[4.5]dec-8-yl]-6-[1-(dimethylamino)ethyl]-7-methylpyrazolo[1,5-a]pyrimidine-3-carboxamide (racemate)

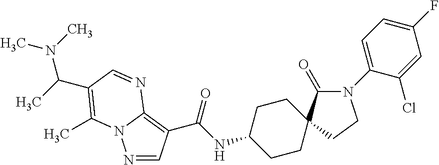

To a mixture of 6-acetyl-N-[2-(2-chloro-4-fluorophenyl)-1-oxo-2-azaspiro[4.5]dec-8-yl]-7-methylpyrazolo[1,5-a]pyrimidine-3-carboxamide (isomer 1) (633 mg, 1.27 mmol) and N-methylmethanamine (1.3 ml, 2.0 M, 2.5 mmol) in THF (6.0 ml) was added titanium (4+) tetrapropan-2-olate (760 μl, 2.5 mmol) and the mixture was stirred at room temperature for 24 hours. (200 mg, 3.18 mmol) was added and the reaction was stirred for 12 h at room temperature. The reaction was poured into a ammonium chloride solution and the mixture was extracted with dichloromethane/2-propanol (4:1). The combined organic phases were washed with water and were filtrated through a hydrophobic filter and concentrated under reduced pressure. The residue was purified by HPLC (Method 9) to give the title compound (70 g).

LC-MS (Method 2): Rₜ=1.25 min; MS (ESIneg): m/z=525 [M−H]⁻

¹H-NMR (400 MHz, DMSO-d6) δ[ppm]: 1.373 (3.51), 1.390 (3.54), 1.671 (1.83), 1.680 (2.07), 1.695 (1.38), 1.702 (1.29), 2.143 (1.20), 2.160 (2.31), 2.178 (3.21), 2.183 (16.00), 2.854 (9.38), 3.610 (1.23), 3.628 (2.25), 3.637 (1.32), 3.645 (1.32), 3.654 (1.05), 7.287 (0.57), 7.294 (0.60), 7.309 (0.81), 7.316 (0.96), 7.329 (0.66), 7.336 (0.69), 7.464 (1.17), 7.478 (1.23), 7.486 (0.99), 7.501 (0.90), 7.576 (1.20), 7.583 (1.26), 7.597 (1.26), 7.604 (1.23), 7.887 (1.02), 7.906 (0.99), 8.553 (6.74), 8.781 (3.99).

### Example 359

### N-{trans-4-[(2-chloro-4-fluorophenyl)carbamoyl]cyclohexyl}-6-(2-hydroxypropan-2-yl)-7-methylpyrazolo[1,5-a]pyrimidine-3-carboxamide

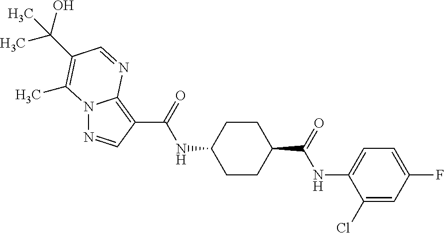 [SMILES: Cc1c(C(C)(C)O)cnc2c(C(=O)N[C@H]3CC[C@H](C(=O)Nc4ccc(F)cc4Cl)CC3)cnn12]

To a solution of ammonium 6-(2-hydroxypropan-2-yl)-7-methylpyrazolo[1,5-a]pyrimidine-3-carboxylate (60.0 mg, 238 μmol) and trans-4-amino-N-(2-chloro-4-fluorophenyl)cyclohexanecarboxamide hydrochloric acid salt (80.4 mg, 262 μmol) in DMF (2.6 ml) was added PyBOP (136 mg, 262 μmol) and N,N-diisopropylethylamine (210 μl, 1.2 mmol) and the reaction was stirred for 12 hours at room temperature. For work-up the reaction was concentrated under reduced pressure. The residue was combined with another batch, which was synthesized accordingly. The combined crude products were purified by HPLC to give the title product (14 mg).

LC-MS (Method 2): Rₜ=1.06 min; MS (ESIpos): m/z=488 [M+H]⁺

¹H-NMR (400 MHz, DMSO-d6) δ[ppm]: 1.755 (16.00), 1.905 (1.56), 1.933 (1.15), 2.193 (6.60), 2.327 (0.72), 2.518 (6.51), 2.523 (5.26), 2.669 (0.74), 3.159 (0.94), 3.172 (0.89), 5.760 (1.25), 7.482 (0.84), 7.489 (0.86), 7.504 (0.88), 7.511 (0.86), 7.575 (0.84), 7.590 (0.91), 7.597 (0.82), 7.612 (0.75), 7.907 (1.17), 9.500 (1.56).

### Example 360

### N-{trans-4-[(2-chloro-4-fluorophenyl)carbamoyl]cyclohexyl}-6-hydroxyimidazo[1,2-b]pyridazine-3-carboxamide

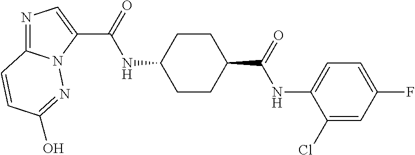 [SMILES: O=C(N[C@H]1CC[C@H](C(=O)Nc2ccc(F)cc2Cl)CC1)c1cnc2ccc(O)nn12]

A mixture of 6-chloro-N-{trans-4-[(2-chloro-4-fluorophenyl)carbamoyl]cyclohexyl}imidazo[1,2-b]pyridazine-3-carboxamide (100 mg, 222 μmol), propan-2-amine (28 μl, 330 μmol) and potassium carbonate (46.0 mg, 333 μmol) in DMSO (2.5 ml) was stirred at 120° C. for 12 hours and for 3 days at room temperature. For work-up the mixture was filtrated. And the residue was purified by HPLC to give after trituration with methanol the title product (9 mg).

¹H-NMR (400 MHz, DMSO-d6) δ[ppm]: 1.107 (0.55), 1.357 (1.16), 1.386 (3.28), 1.415 (3.76), 1.443 (1.78), 1.536 (1.78), 1.564 (4.10), 1.599 (3.90), 1.626 (1.44), 1.907 (3.01), 1.947 (4.58), 1.975 (3.76), 2.038 (4.24), 2.063 (4.03), 2.323 (3.15), 2.327 (4.24), 2.331 (3.21), 2.405 (1.64), 2.433 (3.21), 2.523 (14.15), 2.540 (3.90), 2.665 (3.21), 2.669 (4.31), 2.673 (3.21), 3.811 (1.71), 3.821 (2.05), 3.831 (1.78), 3.840 (1.98), 3.851 (1.57), 6.932 (7.11), 6.956 (7.32), 7.197 (2.26), 7.205 (2.46), 7.219 (3.97), 7.226 (4.17), 7.240 (2.53), 7.247 (2.53), 7.494 (4.38), 7.502 (4.44), 7.516 (4.44), 7.523 (4.24), 7.577 (4.65), 7.592 (4.99), 7.600 (4.51), 7.614 (4.10), 8.030 (16.00), 8.102 (6.29), 8.126 (6.09), 8.610 (2.60), 8.629 (2.53), 9.514 (8.68).

### Example 361

### N-[(trans)-1-oxo-2-(thiophen-2-yl)-2-azaspiro[4.5]dec-8-yl]pyrazolo[1,5-a]pyrimidine-3-carboxamide

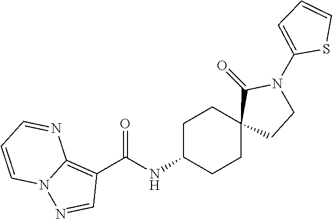 [SMILES: O=C(N[C@H]1CC[C@@]2(CCN(c3cccs3)C2=O)CC1)c1cnn2cccnc12]

To a solution of 8-amino-2-(thiophen-2-yl)-2-azaspiro[4.5]decan-1-one (isomer 1) (66.0 mg, 264 μmol) and pyrazolo[1,5-a]pyrimidine-3-carboxylic acid (53.8 mg, 330 μmol) in DMF (780 μl) was added PyBOP (165 mg, 316 μmol) and N,N-diisopropylethylamine (230 μl, 1.3 mmol) and the reaction was stirred for 16 h at room temperature. For work-up the reaction was poured into water and the resulting precipitate was filtrated. The residue was washed with water and methanol and the crude product triturated with DMSO to give after drying under vacuum the title product (62 mg)

LC-MS (Method 2): Rₜ=0.99 min; MS (ESIneg): m/z=394 [M−H]⁻

¹H-NMR (400 MHz, DMSO-d6) δ[ppm]: 1.461 (1.69), 1.471 (1.69), 1.491 (1.81), 1.500 (1.98), 1.614 (1.35), 1.646 (3.69), 1.659 (2.97), 1.669 (2.64), 1.693 (2.21), 1.701 (2.44), 1.943 (1.95), 1.952 (2.08), 1.963 (1.29), 1.976 (1.95), 1.985 (1.73), 2.164 (3.30), 2.182 (5.38), 2.200 (3.46), 2.327 (0.89), 2.518 (3.02), 2.523 (1.98), 2.540 (16.00), 2.669 (0.89), 3.820 (3.95), 3.828 (1.49), 3.838 (6.28), 3.846 (1.96), 3.855 (4.29), 3.863 (1.24), 6.665 (3.48), 6.669 (3.79), 6.675 (3.95), 6.678 (3.88), 6.906 (3.88), 6.915 (3.36), 6.919 (4.52), 6.929 (4.43), 7.061 (4.48), 7.065 (4.81), 7.075 (4.05), 7.079 (3.68), 7.260 (3.49), 7.271 (3.46), 7.277 (3.51), 7.288 (3.66), 7.817 (2.79), 7.837 (2.71), 8.582 (13.87), 8.819 (4.28), 8.823 (4.40), 8.829 (4.18), 8.833 (3.97), 9.309 (4.35), 9.313 (4.51), 9.326 (4.52), 9.331 (3.94).

### Example 362

### methyl 3-chloro-4-{(trans)-1-oxo-8-[(pyrazolo[1,5-a]pyrimidin-3-ylcarbonyl)amino]-2-azaspiro[4.5]dec-2-yl}benzoate

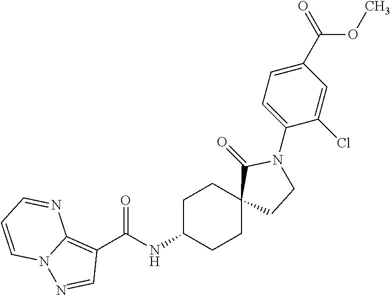 [SMILES: COC(=O)c1ccc(N2CC[C@]3(CC[C@@H](NC(=O)c4cnn5cccnc45)CC3)C2=O)c(Cl)c1]

To a solution of methyl 4-[8-amino-1-oxo-2-azaspiro[4.5]dec-2-yl]-3-chlorobenzoate (isomer 1) (69.0 mg, 205 μmol) and pyrazolo[1,5-a]pyrimidine-3-carboxylic acid (41.8 mg, 256 μmol) in DMF (2.5 ml) was added PyBOP (128 mg, 246 μmol) and N,N-diisopropylethylamine (180 μl, 1.0 mmol) and the reaction was stirred for 3 days at room temperature. For work-up the reaction was poured into ice water and the resulting precipitate was filtrated. The residue was washed with water and methanol to give after drying under vacuum the title product (16 mg).

LC-MS (Method 2): Rₜ=1.05 min; MS (ESIpos): m/z=482 [M+H]⁺

¹H-NMR (400 MHz, DMSO-d6) δ[ppm]: 1.476 (0.62), 1.497 (1.06), 1.527 (1.12), 1.549 (0.45), 1.695 (3.02), 1.709 (2.63), 1.717 (2.57), 1.974 (1.17), 1.997 (1.06), 2.075 (16.00), 2.085 (0.67), 2.177 (1.73), 2.195 (3.36), 2.212 (1.85), 2.323 (2.35), 2.327 (3.24), 2.332 (2.29), 2.456 (1.51), 2.518 (15.50), 2.523 (10.85), 2.665 (2.46), 2.669 (3.36), 2.673 (2.41), 2.728 (1.12), 2.889 (1.34), 3.287 (0.62), 3.368 (0.73), 3.698 (1.85), 3.716 (3.19), 3.733 (1.68), 3.842 (0.56), 3.852 (0.50), 3.861 (0.62), 3.883 (14.15), 7.263 (1.73), 7.273 (1.62), 7.280 (1.62), 7.290 (1.73), 7.595 (2.74), 7.615 (3.02), 7.822 (1.57), 7.841 (1.51), 7.958 (1.96), 7.964 (2.01), 7.979 (1.62), 7.984 (1.79), 8.042 (3.52), 8.047 (2.97), 8.581 (6.60), 8.822 (1.96), 8.826 (2.07), 8.832 (1.90), 8.837 (1.85), 9.311 (2.13), 9.316 (2.01), 9.329 (2.07), 9.333 (1.90).

### Example 363

### N-{trans-4-[(2-chloro-4,6-difluorophenyl)carbamoyl]cyclohexyl}-5-methyl-7,8-dihydro-6H-cyclopenta[e]pyrazolo[1,5-a]pyrimidine-3-carboxamide

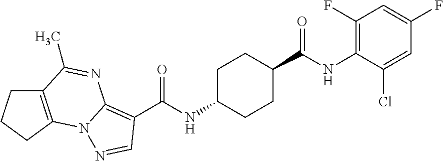 [SMILES: Cc1nc2c(C(=O)N[C@H]3CC[C@H](C(=O)Nc4c(F)cc(F)cc4Cl)CC3)cnn2c2c1CCC2]

To a solution of trans-4-amino-N-(2-chloro-4,6-difluorophenyl)cyclohexanecarboxamide hydrochloride (80.8 mg, 249 μmol) in DMF (2.0 ml) was added PyBOP (172 mg, 331 μmol),N,N-diisopropylethylamine (190 μl, 1.1 mmol) followed by the addition of 5-methyl-7,8-dihydro-6H-cyclopenta[e]pyrazolo[1,5-a]pyrimidine-3-carboxylic acid (60.0 mg, 276 μmol) and the reaction was stirred for 16 hours at room temperature. For work-up the mixture was poured into water and the resulting precipitate was filtrated. The residue was washed with water and the crude product was purified by HPLC [Instrument: Labomatic Pump HD-5000, Labomatic SP-3000, Labocord 5000, Labomatic Labcol Vario 4000, Gilson GX-241, column: Chiralpak IE 5 μm 250×30 mm Nr.027; Solvent: ethanol/methanol/diethylamine 50:50:0.1 (v/v/v)flow: 30 mL/min; temperature: RT; MWD 254 nm] to give the title product (78 mg).

LC-MS (Method 1): Rₜ=1.13 min; MS (ESIpos): m/z=488 [M+H]⁺

¹H-NMR (400 MHz, DMSO-d6) δ[ppm]: 1.380 (1.03), 1.410 (1.20), 1.578 (1.20), 1.616 (1.14), 1.955 (1.37), 1.983 (1.09), 2.077 (1.26), 2.083 (1.83), 2.101 (1.20), 2.250 (1.37), 2.268 (1.94), 2.287 (1.49), 2.327 (0.80), 2.518 (3.71), 2.523 (2.40), 2.596 (16.00), 2.669 (0.80), 3.015 (1.43), 3.034 (2.29), 3.051 (1.31), 3.377 (2.06), 7.400 (0.80), 7.416 (1.03), 7.424 (1.43), 7.447 (1.83), 7.451 (1.26), 7.459 (0.86), 7.468 (1.03), 7.472 (1.09), 7.479 (0.80), 8.037 (1.66), 8.057 (1.60), 8.458 (7.49), 9.604 (1.83).

### Example 364

### N-[(trans)-2-(2-chloro-4-fluorophenyl)-1-oxo-2-azaspiro[4.5]dec-8-yl]-7-hydroxy-5-methylpyrazolo[1,5-a]pyrimidine-3-carboxamide

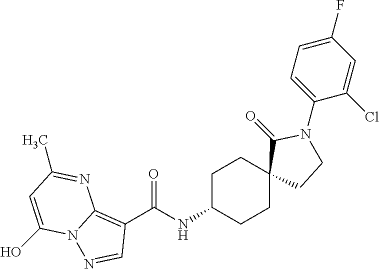 [SMILES: Cc1cc(O)n2ncc(C(=O)N[C@H]3CC[C@@]4(CCN(c5ccc(F)cc5Cl)C4=O)CC3)c2n1]

To a solution of 8-amino-2-(2-chloro-4-fluorophenyl)-2-azaspiro[4.5]decan-1-one (isomer 1) (80.0 mg, 270 μmol) and 7-hydroxy-5-methylpyrazolo[1,5-a]pyrimidine-3-carboxylic acid (65.1 mg, 337 μmol) in DMF (3.0 ml) was added PyBOP (168 mg, 323 μmol) and N,N-diisopropylethylamine (230 μl, 1.3 mmol) and the reaction was stirred for 12 h at room temperature. For work-up the mixture was concentrated under reduced pressure and the residue was treated with methanol. The resulting precipitate was filtrated and the residue was washed with water and methanol. The filtrate was concentrated under reduced pressure and was purified by HPLC to give the title product (3.5 mg)

LC-MS (Method 2): Rₜ=0.78 min; MS (ESIpos): m/z=472 [M+H]⁺

¹H-NMR (400 MHz, DMSO-d6) δ[ppm]: 1.456 (1.43), 1.466 (1.64), 1.487 (1.71), 1.496 (1.86), 1.515 (0.86), 1.527 (0.82), 1.615 (1.07), 1.639 (2.64), 1.648 (2.57), 1.681 (5.50), 1.713 (1.18), 1.880 (2.21), 1.907 (2.71), 2.120 (3.11), 2.138 (5.96), 2.155 (3.29), 2.322 (1.18), 2.326 (1.54), 2.332 (1.18), 2.380 (16.00), 2.522 (6.11), 2.664 (1.14), 2.668 (1.54), 2.673 (1.18), 3.158 (0.61), 3.171 (0.64), 3.619 (3.21), 3.636 (5.68), 3.653 (3.07), 3.783 (0.96), 3.803 (0.96), 5.742 (4.18), 7.289 (1.18), 7.296 (1.29), 7.311 (2.32), 7.317 (2.46), 7.331 (1.50), 7.339 (1.54), 7.459 (2.79), 7.473 (2.96), 7.480 (2.43), 7.495 (2.18), 7.578 (2.82), 7.585 (2.89), 7.600 (2.89), 7.607 (2.79), 8.184 (2.18), 8.203 (2.14), 8.361 (6.75), 11.703 (3.39).

### Example 365

### N-{4-[(2-chloro-4-fluorophenyl)carbamoyl]-4-fluorocyclohexyl}pyrazolo[1,5-a]pyrimidine-3-carboxamide (single isomer)

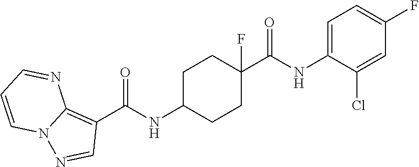 [SMILES: O=C(NC1CCC(F)(C(=O)Nc2ccc(F)cc2Cl)CC1)c1cnn2cccnc12]

1-Chloro-1-dimethylamino-2-methyl-1-propen (180 μl, 1.5 mmol) was added to a solution of 1-fluoro-4-[(pyrazolo[1,5-a]pyrimidin-3-ylcarbonyl)amino]cyclohexanecarboxylic acid (149 mg, 486 μmol) in dichloromethane (9.2 ml) and the mixture was stirred for 30 minutes at room temperature. 2-chloro-4-fluoroaniline (120 μl, 970 μmol) and pyridine (200 μl, 2.4 mmol) were added and the mixture was stirred for 16 h at room temperature in a microwave reactor. For work-up, the reaction mixture was poured into water and the mixture was extracted with dichloromethane/i-propanol (4:1). The combined organic phases were washed with sodium bicarbonate solution and water the organic phase was filtrated through a hydrophobic filter and was then concentrated under reduced pressure. The crude product was purified by HPLC (Method 8) to give the title compound (142 g) as a single isomer.

LC-MS Method 1): Rₜ=1.13 min; MS (ESIpos): m/z=434 [M+H]⁺

¹H-NMR (400 MHz, DMSO-d6) δ[ppm]: 1.880 (3.51), 1.912 (4.61), 1.923 (3.90), 1.946 (6.30), 1.966 (3.20), 1.976 (3.03), 2.140 (1.30), 2.169 (1.69), 2.211 (1.50), 2.249 (1.66), 2.523 (6.00), 4.219 (1.89), 7.231 (2.05), 7.239 (2.36), 7.250 (5.60), 7.253 (4.13), 7.260 (8.28), 7.267 (5.06), 7.274 (3.33), 7.277 (5.60), 7.281 (3.21), 7.516 (4.00), 7.531 (4.49), 7.538 (7.92), 7.546 (4.70), 7.553 (4.00), 7.560 (4.69), 7.568 (4.41), 8.175 (3.32), 8.193 (3.30), 8.588 (16.00), 8.787 (5.28), 8.791 (5.69), 8.798 (5.53), 8.802 (5.06), 9.312 (5.15), 9.316 (5.54), 9.330 (5.55), 9.334 (5.09), 9.799 (3.74), 9.808 (3.69).

### Example 366

### N⁴-{4-[(2-chloro-4-fluorophenyl)carbamoyl]-4-fluorocyclohexyl}-N⁵-methyl-1H-imidazole-4,5-dicarboxamide (single isomer)

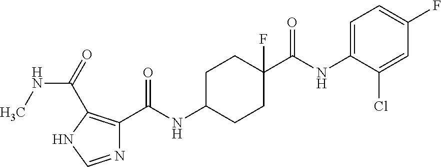 [SMILES: CNC(=O)c1[nH]cnc1C(=O)NC1CCC(F)(C(=O)Nc2ccc(F)cc2Cl)CC1]

1-Chloro-1-dimethylamino-2-methyl-1-propen (330 μl, 2.7 mmol) was added to a solution of 1-fluoro-4-({[5-(methylcarbamoyl)-1H-imidazol-4-yl]carbonyl}amino)cyclohexane carboxylic acid (214 mg, 685 μmol) in dichloromethane (13 ml, 200 mmol) and the mixture was stirred for 30 minutes at room temperature. 2-chloro-4-fluoroaniline (250 μl, 2.1 mmol) and pyridine (330 μl, 4.1 mmol) were added and the mixture was stirred for 16 h at room temperature in a microwave reactor. For work-up, the reaction mixture was poured into water and the mixture was extracted with dichloromethane/i-propanol (4:1). The combined organic phases were washed with sodium bicarbonate solution and water the organic phase was filtrated through a hydrophobic filter and was then concentrated under reduced pressure. The crude product was purified by HPLC (Method 8) to give the title compound (96 g) as a single isomer.

LC-MS (Method 1): Rₜ=1.12 min; MS (ESIpos): m/z=440 [M+H]⁺

¹H-NMR (400 MHz, DMSO-d6) δ[ppm]: 1.890 (9.46), 2.074 (2.41), 2.180 (1.42), 2.202 (1.15), 2.263 (1.39), 2.323 (1.60), 2.327 (2.08), 2.331 (1.51), 2.518 (15.88), 2.523 (13.08), 2.665 (1.48), 2.669 (1.99), 2.674 (1.39), 2.779 (12.87), 2.791 (12.96), 4.098 (1.90), 7.231 (2.14), 7.238 (2.41), 7.251 (3.13), 7.260 (3.59), 7.273 (2.56), 7.280 (2.71), 7.521 (4.10), 7.536 (5.24), 7.538 (5.91), 7.545 (6.51), 7.560 (6.30), 7.567 (4.73), 7.803 (16.00), 9.741 (2.74).

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate)
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide); tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

### Sterile IV Solution:

A 5 mg/ml solution of the desired compound of this invention can be made using sterile, injectable water, and the pH is adjusted if necessary. The solution is diluted for administration to 1-2 mg/ml with sterile 5% dextrose and is administered as an IV infusion over about 60 min.

### Lyophilised Powder for IV Administration:

A sterile preparation can be prepared with (i) 100-1000 mg of the desired compound of this invention as a lyophilised powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 min.

### Intramuscular Suspension:

The following solution or suspension can be prepared, for intramuscular injection:

50 mg/ml of the desired, water-insoluble compound of this invention
5 mg/ml sodium carboxymethylcellulose
4 mg/ml TWEEN 80
9 mg/ml sodium chloride
9 mg/ml benzyl alcohol

### Hard Shell Capsules:

A large number of unit capsules are prepared by filling standard two-piece hard galantine capsules each with 100 mg of powdered active ingredient, 150 mg of lactose, 50 mg of cellulose and 6 mg of magnesium stearate.

### Soft Gelatin Capsules:

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets:

A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg of starch, and 98.8 mg of lactose. Appropriate aqueous and non-aqueous coatings may be applied to increase palatability, improve elegance and stability or delay absorption.

### Immediate Release Tablets/Capsules:

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

The term “chemotherapeutic anti-cancer agents”, includes but is not limited to

131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-linked lintuzumab, EM-1421, HyperAcute vaccine, tucotuzumab celmoleukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131I-chTNT-1/B. Monoclonal antibodies useful as the protein therapeutic include, but are not limited to, muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

A compound of general formula (I) as defined herein can optionally be administered in combination with one or more of the following: ARRY-162, ARRY-300, ARRY-704, AS-703026, AZD-5363, AZD-8055, BEZ-235, BGT-226, BKM-120, BYL-719, CAL-101, CC-223, CH-5132799, deforolimus, E-6201, enzastaurin, GDC-0032, GDC-0068, GDC-0623, GDC-0941, GDC-0973, GDC-0980, GSK-2110183, GSK-2126458, GSK-2141795, MK-2206, novolimus, OSI-027, perifosine, PF-04691502, PF-05212384, PX-866, rapamycin, RG-7167, RO-4987655, RO-5126766, selumetinib, TAK-733, trametinib, triciribine, UCN-01, WX-554, XL-147, XL-765, zotarolimus, ZSTK-474.

Generally, the use of cytotoxic and/or cytostatic agents in combination with a compound or composition of the present invention will serve to:

(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

### Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated with one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or other induction of DNA damage in the cell. In another aspect of the invention, a compound of the invention is administered to a cell concomitantly with the radiation or other induction of DNA damage in the cell. In yet another aspect of the invention, a compound of the invention is administered to a cell immediately after radiation or other induction of DNA damage in the cell has begun.

In another aspect, the cell is in vitro. In another embodiment, the cell is in vivo.

As mentioned supra, the compounds of the present invention have surprisingly been found to effectively inhibit tankyrases and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses are affected by inhibition of tankyrases, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra or a stereoisomer, a tautomer, an N-oxide, a hydrate, a solvate, or a salt thereof, particularly a pharmaceutically acceptable salt thereof, or a mixture of same. for manufacturing a pharmaceutical composition for the treatment or prophylaxis of a disease.

Another aspect of the present invention is the use of a compound of formula (I) or a stereoisomer, a tautomer, an N-oxide, a hydrate, a solvate, or a salt thereof, particularly a pharmaceutically acceptable salt thereof, or a mixture of same, as described herein, in the manufacture of a medicament for the treatment or prophylaxis of a disease.

The diseases referred to in the three preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

Diseases further included in the context of the present invention are metabolic diseases (e.g. diabetes and obesity), fibrosis (e.g. lung fibrogenesis) and viral infection.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyperproliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

### Biological Assays:

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch(es).

### 6 Biochemical Assays

### 6.1 TNKS1 Assays

### TNKS1 Assay A

The potency of the compounds according to the invention was assessed by applying an in vitro inhibition assay. The TNKS1 catalyzed NAD⁺-dependent ribosylation of a suitable protein substrate was detected using a commercially available biotin/streptavidin binding based assay format [TNKS1 Histone Ribosylation Assay Kit (Biotin-labeled NAD⁺), Catalog #80579; BPS Bioscience, San Diego, USA]. Here, the incorporation of a biotin-labeled NAD+during the TNKS1 catalyzed ribosylation reaction was detected with a streptavidin-HRP coupled chemi-luminescent readout. The intensity of the readout signal is proportional to the incorporated NAD⁺. Inhibition of TNKS1 leads to a decreased incorporation of NAD⁺ and consequently to a lower readout signal intensity. The concentration of a test compound which inhibits the enzyme activity by 50% (corresponds to half of the normed readout signal intensity) is reported as IC₅₀.

### Protocol

The assay was conducted in a 384 well MTP format according to the manufacturer's protocol [http://www.bpsbioscience.com/poly-adp-ribose-polymerase/assay-kit/tnks1-histone-ribosylation-assay-kit-biotin-labeled-nad-80579 referencing: Brown, J. A., Marala, R. B. J. Pharmacol. Toxicol. Methods 2002 47:137] and using a BMG Pherastar MTP reader [BMG-Labtech, Offenburg, Germany].

### TNKS1 Assay B

The potency of selected compounds according to the invention was assessed applying a modified in vitro inhibition assay. Here, the TNKS1 catalyzed NAD⁺-dependent ribosylation of the enzyme itself (auto-parsylation) was detected using [³H]-NAD⁺ as substrate and applying the scintillation proximity assay (SPA) method to detect tritium-labeled, parsylated TNKS1. The intensity of the readout signal is proportional to the incorporated [³H]-NAD⁺. Inhibition of TNKS1 leads to a decreased incorporation of [³H]-NAD⁺ and consequently to a lower readout signal intensity. The concentration of a test compound which inhibits the enzyme activity by 50% (corresponds to half of the normed readout signal intensity) is reported as IC₅₀.

### Protocol Auto-Parsylation Assay

The assay was conducted in a 96 well MTP format with the identical TNKS1 enzyme sample and NAD⁺ sample as in the histone ribosylation assay with the following modifications: TNKS1 enzyme sample was diluted with a modified assay buffer (50 mM MES pH 7.0, 1 mM DTT, 0.01% Triton X-100) to a final concentration of 6 nM TNKS1 and 10×NAD⁺ solution was diluted with the modified assay buffer (s. above) to a final 0.445×NAD⁺ solution doped with 100 Bq/μl [³H]-NAD⁺[Catalog #NET443H050UC, Perkin Elmer, Waltham, Mass., USA]. Substrate solution (10 μl) was incubated with different test compound concentrations (2.5 ul in 10% DMSO in modified assay buffer) or control (2.5 ul 10% DMSO in modified assay buffer only) and enzyme (10 μl) over night at room temperature. Incorporated tritium was measured after addition of 50 μl SPA beads (1 mg/ml) [Catalog #RPNQ0095 20 mg/ml, Perkin Elmer, Waltham, Mass., USA; diluted 1:10 with Dulbecco's phosphate buffered saline, PBS Catalog #D8537, Sigma-Aldrich, Steinheim, Germany] and detection of the photon emission with a beta count plate reader [Wallac MicroBeta®, Perkin Elmer, Waltham, Mass., USA].

### 6.2 TNKS2 Assays

### TNKS2 Assay A

The potency of the compounds according to the invention was assessed applying an in vitro inhibition assay. The TNKS2 catalyzed NAD⁺-dependent ribosylation of a suitable protein substrate was detected using a commercially available biotin/streptavidin binding based assay format [TNKS2 Histone Ribosylation Assay Kit (Biotin-labeled NAD⁺), Catalog #80572; BPS Bioscience, San Diego, USA]. Here, the incorporation of a biotin-labeled NAD⁺ during the TNKS2 catalyzed ribosylation reaction was detected with a streptavidin-HRP coupled chemi-luminescent readout. The intensity of the readout signal is proportional to the incorporated NAD⁺. Inhibition of TNKS2 leads to a decreased incorporation of NAD⁺ and consequently to a lower readout signal intensity. The concentration of a test compound which inhibits the enzyme activity by 50% (corresponds to half of the normed readout signal intensity) is reported as IC₅₀.

### Protocol

The assay was conducted in a 384 well MTP format according to the manufacturer's protocol [http://www.bpsbioscience.com/poly-adp-ribose-polymerase/assay-kit/tnks2-histone-ribosylation-assay-kit-biotin-labeled-nad-80572 referencing: Brown, J. A., Marala, R. B. J. Pharmacol. Toxicol. Methods 2002 47:137]. and using a BMG Pherastar MTP reader [BMG-Labtech, Offenburg, Germany].

### TNKS2 Assay B

The potency of selected compounds according to the invention was assessed applying a modified in vitro inhibition assay. Here, the TNKS2 catalyzed NAD⁺-dependent ribosylation of the enzyme itself (auto-parsylation) was detected using [³H]-NAD⁺ as substrate and applying the scintillation proximity assay (SPA) method to detect tritium-labeled, parsylated TNKS2. The intensity of the readout signal is proportional to the incorporated [³H]-NAD⁺. Inhibition of TNKS2 leads to a decreased incorporation of [³H]-NAD⁺ and consequently to a lower readout signal intensity. The concentration of a test compound which inhibits the enzyme activity by 50% (corresponds to half of the normed readout signal intensity) is reported as IC₅₀.

### Protocol Auto-Parsylation Assay

The assay was conducted in a 96 well MTP format with the identical TNKS2 enzyme sample and NAD⁺ sample as in the histone ribosylation assay with the following modifications: TNKS2 enzyme sample was diluted with a modified assay buffer (50 mM MES pH 7.0, 1 mM DTT, 0.01% Triton X-100) to a final concentration of 6 nM TNKS2 and 10×NAD⁺ solution was diluted with the modified assay buffer (s. above) to a final 0.445×NAD⁺ solution doped with 100 Bq/μl [³H]-NAD⁺[Catalog #NET443H050UC, Perkin Elmer, Waltham, Mass., USA]. Substrate solution (10 μl) was incubated with different test compound concentrations (2.5 μl in 10% DMSO in modified assay buffer) or control (2.5 μl 10% DMSO in modified assay buffer only) and enzyme (10 μl) over night at room temperature. Incorporated tritium was measured after addition of 50 ul SPA beads (1 mg/ml) [Catalog #RPNQ0095 20 mg/ml, Perkin Elmer, Waltham, Mass., USA; diluted 1:10 with Dulbecco's phosphate buffered saline, PBS Catalog #D8537, Sigma-Aldrich, Steinheim, Germany] and detection of the photon emission with a beta count plate reader [Wallac MicroBeta®, Perkin Elmer, Waltham, Mass., USA].

### 6.3 PARP1 Assay

The potency of the compounds according to the invention was assessed using a commercially available biotin/streptavidin binding assay kits from BPS Bioscience, San Diego, USA (Catalog #80551). The incorporation of a biotin-labeled NAD⁺ during the PARP1 catalyzed ribosylation of a suitable protein substrate was detected using with a streptavidin-HRP coupled chemi-luminescent readout. The intensity of the readout signal is proportional to the incorporated NAD⁺. Inhibition of PARP1 leads to a decreased incorporation of NAD+ and consequently to a lower readout signal intensity. The concentration of a test compound that inhibits the enzyme activity by 50% (corresponds to half of the normed readout signal intensity) is reported as IC₅₀.

### Protocol

The assay was conducted in a 96 well MTP format according to the manufacturer's protocol (Catalog No. 80551) and using a BMG Pherastar MTP reader [BMG-Labtech, Offenburg, Germany].

### 6.4 PARP2 Assay

The potency of the compounds according to the invention was assessed using a commercially available biotin/streptavidin binding assay kits from BPS Bioscience, San Diego, USA (Catalog #80551). The incorporation of a biotin-labeled NAD⁺ during the PARP2 catalyzed ribosylation of a suitable protein substrate was detected using with a streptavidin-HRP coupled chemi-luminescent readout. The intensity of the readout signal is proportional to the incorporated NAD⁺. Inhibition of PARP2 leads to a decreased incorporation of NAD⁺ and consequently to a lower readout signal intensity. The concentration of a test compound that inhibits the enzyme activity by 50% (corresponds to half of the normed readout signal intensity) is reported as IC₅₀.

### Protocol

The assay was conducted in a 96 well MTP format according to the manufacturer's protocol (Catalog No. 80552) and using a BMG Pherastar MTP reader [BMG-Labtech, Offenburg, Germany].

### 7 Cellular Assays

### 7.1 Measurement of the Inhibitory Activity of Selected Compounds on the Wildtype Wnt Signaling Cascade: HEK293 TOP/FOP Assay

In order to discover and characterize small molecules which inhibit the wildtype Wnt pathway, a cellular reporter assay was employed. The corresponding assay cell was generated by transfection of the mammalian cell line HEK293 (ATCC, #CRL-1573) with the Super TopFlash vector (Morin, Science 275, 1997, 1787-1790; Molenaar et al., Cell 86 (3), 1996, 391-399). The HEK293 cell line is cultivated at 37° C. and 5% CO₂ in DMEM (Life Technologies, #41965-039), supplemented with 2 mM glutamine, 20 mM HEPES, 1.4 mM pyruvate, 0.15% Na-bicarbonate and 10% foetal bovine serum (GIBCO, #10270). Stable transfectants were generated by selection with 300 μg/ml Hygromycin.

In a parallel approach, HEK293 cells were cotransfected with the FOP control vector and pcDNA3. The FOP vector is identical to the TOP construct, but it contains instead of functional TCF elements a randomized, non-functional sequence. For this transfection a stable transfected cell line was generated as well, based on selection with Geneticin (1 mg/ml).

In preparation of the assay, the two cell lines were plated 24 h before beginning the test at 10000 cells per well in a 384 micro titre plate (MTP) in 30 μl growth medium. Before compound testing a dose response curve for the Wnt dependent luciferase expression was recorded by stimulating the assay cell line with human recombinant Wnt-3a (R&D, #5036-WN-010) at different concentrations for 16 h at 37° C. and 5% CO₂ followed by subsequent luciferase measurement, to determine the Wnt-3a EC₅₀ for the HEK293 TOP cell line on the day of testing. The recombinant human Wnt-3a was thereby applied between 2500 and 5 ng/ml in two-fold dilution steps.

Selective inhibitory activity for small molecules on the wildtype Wnt pathway was determined after parallel incubation of both (TOP and FOP) HEK293 reporter cell lines with a compound dilution series from 50 μM to 15 nM in steps of 3.16-fold dilutions in CAFTY buffer (130 mM sodium chloride, 5 mM potassium chloride, 20 mM HEPES, 1 mM magnesium chloride, 5 mM sodium bicarbonate, pH 7.4) containing 2 mM Ca²⁺ and 0.01% BSA.

The compounds were thereby serially prediluted in 100% DMSO and thereafter 50 fold into the CAFTY compound dilution buffer (described above). From this dilution 10 μl were added in combination with the EC₅₀ concentration of recombinant Wnt3a to the cells in 30 μl growth medium and incubated for 16 hours at 37° C. and 5% CO₂. Thereafter luciferase assay buffer (1:1 mixture of luciferase substrate buffer (20 mM Tricine, 2.67 mM magnesium sulfate, 0.1 mM EDTA, 4 mM DTT, 270 μM Coenzyme A, 470 μM Luciferin, 530 μM ATP, ph adjusted to pH 7.8 with a sufficient volume of 5M sodium hydroxide) and Triton buffer (30 ml Triton X-100, 115 ml glycerol, 308 mg Dithiothreitol, 4.45 g disodium hydrogen phosphate dihydrate, 3.03 g Tris. HCl, ad 11H₂O, pH 7.8) was added in an equal volume to determine luciferase expression as a measure of Wnt signaling activity in a luminometer. The Wnt inhibitory activity was determined as IC₅₀ of resulting dose response curves.

### 7.2 Axin Stabilization Assay

The in vitro and in vivo effect of Tankyrase inhibition on the stabilization of cellular Axin was assessed using Peggy Simple Western assay with size-based separation and immunodetection of Axin2. SW403 cells (but not limited to) were seeded at 50000 cells per well in 96-well plates. After overnight incubation, cells were treated with testing compounds and vehicle at 37° C. for 24 hours. Thereafter, cell s were washed with PBS and then lysed in 15 μl of lysis buffer (M-PER buffer, Thermo Scientific #78505) with complete proteinase and phosphatase inhibitors (Roche, #11836153001 and #04906837001). The lysates were centrifuged and the supernatants were harvested for analysis. Tumor xenografts from in vivo studies were homogenized in a 2 ml tubes of Precellys®24 (Bertin Technologies, Villeurbanne, France) following with centrafugation to obtain tumor lysates. Capillary electrophoresis-based Simple Western assays were carried out with Peggy Sue™ NanoPro 1000 (ProteinSimple, California, USA). The protein amounts of Axin2 (but not limited to) were detected using anti-Axin2 antibody (Cell Signaling, Catalog #2151), quantified using the area under the curve, and normalized against GAPDH (anti-GAPDH, Zytomed Systems GmbH, Catalog #RGM2-6C5, Berlin, Germany).

### 7.3 Real-Time RT-PCR for Quantitative Analysis of Gene Transcription

Real-time RT-PCR using a TaqMan fluorogenic detection system is a simple and sensitive assay for quantitative analysis of gene transcription. The TaqMan fluorogenic detection system can monitor PCR in real time using a dual-labeled fluorogenic hybridization probe (TaqMan probe) and a polymerase with 5′-3′ exonuclease activity.

Cells from different cancer cell lines (as HCT116, but not limited to) were grown at 500-1000 cells/well in 384 well cell culture plates. For cell lysis the cell medium was carefully removed. The cells were washed carefully once with 50 μl/well PBS. Then 9.75 μl/well cell lysis buffer (50 mM Tris HCl pH 8.0, 40 mM sodium chloride, 1.5 mM magnesium chloride, 0.5% IGEPAL CA 630, 50 mM Guanidium thiocyanate) and 0.25 μl RNASeOUT (40 U/μl, Invitrogen, 10777-019)) per well were added. The plate was incubated for 5 min at room temperature. Then 30 μl DNAse/RNAse-free water per well was added and the lysates mixed. Isolation of total RNA from tumor tissues was conducted using InviTrap® Spin Tissue RNA Mini Kit (#1062100300, STRATEC MOLECULAR).

For the One-Step RT-PCR 2 μl lysate (each) was transferred to a 384 well PCR plate. The PCR reaction was composed by 5 μl 2× One Step RT qPCR MasterMix Plus, 0.05 μl Euroscript RT/RNAse Inhibitor (50 U/μl, 20 U/μl) and 200 nM of the appropriate Primer/Hydrolysis Probe mix (primer sequences of forward, reverse and probe are given below for each analysed gene of interest or house keeping gene). 10 μl water were added per well. The plate was sealed with an adhesive optical film. The RT-PCR protocol was setup with 30 min 48° C., then 10 min 95° C. followed by 50 cycles of 15 sec 95° C./1 min 60° C. and a cooling step of 40° C. for 30 sec using a Lightcycler LS440 from Roche. Relative expression was calculated using CP values from the gene of interest (e.g. AXIN2, but not limited to) and a house keeping gene (L32).

### Used Primers

L32 (forward primer: AAGTTCATCCGGCACCAGTC
(SEQ ID NO. 1); reverse primer: TGGCCCTTGAATCTT
CTACGA (SEQ ID NO. 2); probe: CCCAGAGGCATTGACAA
CAGGG (SEQ ID NO. 3))
AXIN2 (forward primer: AGGCCAGTGAGTTGGTTGTC
(SEQ ID NO. 4); reverse primer: AGCTCTGAGCCTTCA
GCATC (SEQ ID NO. 5); probe: TCTGTGGGGAAGAAATTC
CATACCG (SEQ ID NO. 6))

### 8 In Vivo Efficacy in Xenograft Models

Subcutaneous xenograft models in immunocompromised mice were used to evaluate in vivo anti-tumor efficacy of the compounds.

### 8.1 Maximum Tolerable Dose (MTD) Studies

Prior to efficacy studies, the maximal tolerable dose (MTD) was determined by the following protocol: Female nude mice (NMRI (nu/nu), Taconic M&B A/S) received a defined oral dose of the test compound daily or bi-daily for 7 consecutive days followed by a 7 day observation period without dosing. Individual body weight and lethality were monitored daily.

The MTD is defined as the maximal applicable dose with a) no animal losing more than 10% body weight compared to initial body weight and b) no lethality during treatment phase.

### 8.2 In Vivo Efficacy Studies

To measure anti-tumor efficacy, the test compounds were analysed in xenograft models on mice. Test compounds were dosed orally at their respective MTD as well as at sub-MTD dosages. In case the MTD could not be determined in previous MTD studies, the compounds were dosed at a maximum daily dose of 200 mg/kg (applied either in one single dose or split in 2 doses at 100 mg/kg).

Compounds were primarily analyzed in an ovarian teratocarcinoma model (PA—1) and in various colorectal cancer models on female immunocompromised mice.

For this purpose, 1-5×10⁶ tumor cells (suspended in 0.1 ml of 50% cell culture medium/50% Matrigel) were subcutanously injected into the flank of each animal. Animals were randomized into treatment groups when tumors had reached an average area of 20-30 mm² and treatment was started. Body weight and tumor area of each animal were measured 2-3 times weekly, depending on tumor growth. Studies were terminated, when animals in the control groups (receiving only compound vehicle solutions) or treatment groups reached tumor areas ˜150 mm². At that time point, all groups in the study were terminated, tumors were isolated and weighed.

As primary parameter for anti-tumor efficacy the Treatment/Control (T/C) ratio of the final tumor weights were calculated (mean tumor weight of treatment group divided by mean tumor weight of vehicle group).

### 8.3 In Vivo Mode of Action Studies

To determine in vivo Mode of Action (MoA) of the test compounds, the same in vivo models as described under 8.2 were utilized. Tumor-bearing animals were treated for at least 3 days at MTD and also sub-MTD dosages. At study end, tumors were isolated and snap frozen in liquid nitrogen. Total RNA and protein were isolated from tumor samples following standard protocols.

Wnt/β-catenin target gene expression and Axin2 protein abundance were measured by standard qRT-PCR and Western blotting methods (see 7.2 and 7.3).

TABLE 1
IC₅₀ values for selected examples in cellular HEK293 TOP
and FOP assay as well as in TNKS1 and TNKS 2 biochemical assay

| | HEK293 | HEK293 | | |
|---|---|---|---|---|
| | TOP | FOP | TNKS1 Assay B | TNKS2 Assay B |
| Example | IC₅₀ [μM] | IC₅₀ [μM] | IC₅₀ [μM] | IC₅₀ [μM] |
| 1 | 0.052 | 50 | 0.073 | 0.072 |
| 2 | 0.025 | 50 | 0.55 | 0.23 |
| 3 | 0.013 | 50 | 0.01 | 0.0081 |
| 4 | 0.01 | 50 | 0.0022 | 0.0021 |
| 5 | 0.19 | 50 | 0.15 | 0.11 |
| 6 | 0.055 | 50 | 0.077 | 0.06 |
| 7 | 0.03 | 50 | 0.024 | 0.027 |
| 8 | 2.1 | 50 | 4.1 | 2.3 |
| 9 | 0.18 | 50 | 0.085 | 0.11 |
| 10 | 0.09 | 50 | 0.25 | 0.24 |
| 11 | 0.14 | 50 | 0.26 | 0.21 |
| 12 | 0.072 | 50 | 0.0037 | 0.0072 |
| 13 | 0.095 | 50 | 0.01 | 0.019 |
| 14 | 0.12 | 50 | 0.063 | 0.037 |
| 15 | 0.071 | 50 | 0.052 | 0.042 |
| 16 | 1.4 | 50 | 1.1 | 1.2 |
| 17 | 0.049 | 50 | 0.084 | 0.09 |
| 18 | 0.15 | 50 | 0.094 | 0.12 |
| 19 | 0.089 | 50 | 0.12 | 0.16 |
| 20 | 0.22 | 50 | 0.09 | 0.13 |
| 21 | 0.079 | 50 | 0.037 | 0.083 |
| 22 | 0.024 | 50 | 0.01 | 0.0053 |
| 23 | 0.11 | 50 | 0.021 | 0.025 |
| 24 | 0.086 | 50 | 0.021 | 0.017 |
| 25 | 0.063 | 50 | 0.013 | 0.011 |
| 26 | 0.14 | 50 | 0.062 | 0.08 |
| 27 | 0.035 | 50 | 0.03 | 0.019 |
| 28 | 0.024 | 50 | 0.068 | 0.026 |
| 29 | 0.93 | 50 | 0.65 | 0.59 |
| 30 | 44 | 40 | >10 | >10 |
| 31 | 0.25 | 50 | 0.15 | 0.12 |
| 32 | 0.27 | 50 | 0.36 | 0.28 |
| 33 | 0.7 | 50 | 0.72 | 0.38 |
| 34 | 0.21 | 50 | | |
| 35 | 17 | 50 | >10 | >10 |
| 36 | 4.9 | 50 | >10 | >10 |
| 37 | 43 | 50 | >10 | >10 |
| 38 | 0.44 | 50 | | |
| 39 | | | 0.38 | 0.1 |
| 40 | 0.46 | 50 | | |
| 41 | 21 | 50 | >10 | >10 |
| 42 | 0.12 | 50 | 0.12 | 0.12 |
| 43 | 0.13 | 50 | 0.081 | 0.074 |
| 44 | 0.081 | 50 | 0.048 | 0.15 |
| 45 | 0.16 | 50 | 0.31 | 0.13 |
| 46 | 0.097 | 50 | 0.065 | 0.025 |
| 47 | 0.39 | 16 | 0.53 | 0.42 |
| 48 | 0.41 | 14 | 0.41 | 0.31 |
| 49 | 0.68 | 32 | 0.4 | 0.13 |
| 50 | 0.35 | 50 | 0.11 | 0.17 |
| 51 | 0.29 | 50 | 0.083 | 0.031 |
| 52 | 0.15 | 40 | 0.022 | 0.011 |
| 53 | 0.4 | 50 | 0.15 | 0.047 |
| 54 | 0.62 | 50 | 0.46 | 0.68 |
| 55 | 0.28 | 50 | 0.74 | 0.49 |
| 56 | 0.99 | 50 | 0.84 | 0.37 |
| 57 | 0.31 | 50 | 0.44 | 0.16 |
| 58 | 1.1 | 50 | 0.74 | 0.71 |
| 59 | 1.3 | 50 | 0.95 | 0.5 |
| 60 | 0.62 | 24 | >10 | 2.2 |
| 61 | 0.021 | 50 | 0.12 | 0.034 |
| 62 | 12 | 50 | >10 | 2 |
| 63 | 0.054 | 50 | 0.057 | 0.031 |
| 64 | 0.74 | 27 | 0.059 | 0.049 |
| 65 | 0.23 | 50 | 0.17 | 0.19 |
| 66 | 0.25 | 50 | 0.31 | 0.28 |
| 67 | 0.22 | 50 | 0.25 | 0.28 |
| 68 | 0.12 | 33 | 0.06 | 0.079 |
| 69 | 0.17 | 50 | 0.057 | 0.057 |
| 70 | 0.22 | 29 | 0.078 | 0.076 |
| 71 | 0.1 | 23 | 0.18 | 0.066 |
| 72 | 0.14 | 50 | 0.055 | 0.026 |
| 73 | 0.4 | 19 | 0.27 | 0.19 |
| 74 | 0.19 | 32 | 0.084 | 0.073 |
| 75 | 0.37 | 50 | 0.084 | 0.1 |
| 76 | 0.23 | 50 | 0.094 | 0.078 |
| 77 | 0.27 | 50 | 0.037 | 0.06 |
| 78 | 0.13 | 9.7 | 0.075 | 0.089 |
| 79 | 0.14 | 43 | 0.17 | 0.1 |
| 80 | 0.09 | 23 | 0.062 | 0.05 |
| 81 | 0.15 | 6.8 | 0.1 | 0.072 |
| 82 | 0.38 | 50 | 0.3 | 0.25 |
| 83 | 0.24 | 29 | 0.61 | 0.27 |
| 84 | 0.23 | 50 | 0.37 | 0.23 |
| 85 | 0.13 | 24 | 0.049 | 0.023 |
| 86 | 0.094 | 14 | 0.03 | 0.022 |
| 87 | 0.06 | 50 | 0.027 | 0.051 |
| 88 | 2.4 | 50 | 3.6 | 7.1 |
| 89 | 1.2 | 24 | 1.2 | 1.6 |
| 90 | 1.9 | 33 | 3.1 | 2.7 |
| 91 | 1.8 | 50 | 2.8 | 6.8 |
| 92 | 3.2 | 21 | >10 | 10 |
| 93 | 1.5 | 12 | 0.79 | 1.6 |
| 94 | 0.61 | 50 | 1.3 | 1.6 |
| 95 | 2.1 | 39 | 2.4 | 4.3 |
| 96 | 0.022 | 18 | 0.04 | 0.028 |
| 97 | 0.034 | 18 | 0.045 | 0.039 |
| 98 | 0.57 | 50 | 0.054 | 0.039 |
| 99 | 0.051 | 20 | 0.068 | 0.11 |
| 100 | 0.2 | 39 | 0.18 | 0.11 |
| 101 | 0.36 | 35 | 0.16 | 0.1 |
| 102 | 0.12 | 50 | 0.33 | 0.2 |
| 103 | 0.41 | 27 | 0.52 | 0.29 |
| 104 | 0.088 | 50 | 0.063 | 0.066 |
| 105 | 0.24 | 50 | 0.19 | 0.12 |
| 106 | 0.4 | 50 | 0.049 | 0.015 |
| 107 | 0.88 | 50 | 0.12 | 0.074 |
| 108 | 0.18 | 50 | 0.052 | 0.06 |
| 109 | 3 | 50 | 0.062 | 0.038 |
| 110 | 0.27 | 50 | 0.31 | 0.086 |
| 111 | 0.073 | 50 | 0.52 | 0.21 |
| 112 | 0.19 | 50 | 0.15 | 0.068 |
| 113 | 0.094 | 50 | 0.11 | 0.052 |
| 114 | 1.6 | 50 | 0.86 | 0.48 |
| 115 | 0.18 | 50 | 0.16 | 0.19 |
| 116 | 0.33 | 8.7 | 0.58 | 0.59 |
| 117 | 0.22 | 50 | 0.34 | 0.46 |
| 118 | 0.3 | 50 | 0.089 | 0.067 |
| 119 | 0.39 | 50 | 0.56 | 0.61 |
| 120 | 0.14 | 7.1 | 0.21 | 0.26 |
| 121 | 0.03 | 50 | 0.4 | 0.33 |
| 122 | 0.47 | 50 | 0.24 | 0.19 |
| 123 | 0.66 | 50 | 1 | 0.48 |
| 124 | 0.29 | 8.9 | 0.23 | 0.26 |
| 125 | 0.069 | 50 | 0.12 | 0.13 |
| 126 | 0.19 | 50 | 0.082 | 0.065 |
| 127 | 0.22 | 50 | 0.32 | 0.3 |
| 128 | 0.24 | 50 | 0.088 | 0.096 |
| 129 | 0.41 | 39 | 0.045 | 0.034 |
| 130 | 0.022 | 50 | 0.034 | 0.018 |
| 131 | 0.14 | 50 | | |
| 132 | 0.051 | 50 | 0.064 | 0.029 |
| 133 | 0.29 | 50 | 0.14 | 0.055 |
| 134 | 0.14 | 50 | 0.074 | 0.033 |
| 135 | 0.038 | 50 | 0.048 | 0.029 |
| 136 | 0.0012 | 50 | 0.013 | 0.0047 |
| 137 | 5 | 50 | 0.011 | 0.0033 |
| 138 | 0.0061 | 50 | 0.022 | 0.007 |
| 139 | 0.0011 | 50 | 0.011 | 0.0042 |
| 140 | 0.00086 | 50 | 0.012 | 0.0069 |
| 141 | 0.0018 | 50 | 0.011 | 0.0065 |
| 142 | 0.068 | 41 | 0.093 | 0.08 |
| 143 | 0.22 | 33 | 0.21 | 0.15 |
| 144 | 0.25 | 25 | 0.19 | 0.23 |
| 145 | 0.15 | 15 | 0.33 | 0.28 |
| 146 | 0.72 | 29 | 0.15 | 0.1 |
| 147 | 0.054 | 50 | 0.15 | 0.077 |
| 148 | 0.14 | 50 | 3.9 | 1.3 |
| 149 | 0.19 | 50 | 0.29 | 0.17 |
| 150 | 0.08 | 50 | 0.17 | 0.035 |
| 151 | 0.11 | 50 | 0.47 | 0.15 |
| 152 | 0.091 | 50 | | 0.14 |
| 153 | 0.53 | 50 | | 0.17 |
| 154 | 0.13 | 50 | 0.087 | 0.057 |
| 155 | 0.13 | 50 | 0.14 | 0.091 |
| 156 | 0.19 | 50 | 0.39 | 0.23 |
| 157 | 8.3 | 50 | 0.45 | 0.28 |
| 158 | 0.034 | 50 | | 0.15 |
| 159 | 0.014 | 50 | | 0.13 |
| 160 | 0.013 | 50 | 0.008 | 0.0034 |
| 161 | 0.055 | 50 | 0.0041 | 0.0016 |
| 162 | 0.0094 | 40 | 0.013 | 0.0082 |
| 163 | 0.0024 | 50 | 0.013 | 0.0059 |
| 164 | 0.0092 | 50 | 0.037 | 0.028 |
| 165 | 0.0038 | 35 | 0.012 | 0.0063 |
| 166 | 0.00069 | 50 | 0.0074 | 0.0026 |
| 167 | 0.13 | 50 | 0.66 | 0.27 |
| 168 | 0.00023 | 50 | 0.0073 | 0.0031 |
| 169 | 0.033 | 50 | 0.035 | 0.014 |
| 170 | 2 | 50 | 2.4 | 1.5 |
| 171 | 0.45 | 50 | 0.4 | 0.15 |
| 172 | 0.41 | 50 | 0.98 | 0.51 |
| 173 | 0.19 | 50 | 0.2 | 0.18 |
| 174 | 0.079 | 50 | | 0.24 |
| 175 | 1.6 | 1.3 | 10 | 10 |
| 176 | 0.35 | 5.3 | 0.16 | 0.13 |
| 177 | 0.071 | 40 | 0.013 | 0.0092 |
| 178 | 0.56 | 50 | 0.33 | 0.23 |
| 179 | 0.41 | 50 | 0.97 | 0.33 |
| 180 | 7.6 | 50 | >10 | >10 |
| 181 | 9.6 | 24 | >10 | >10 |
| 182 | 0.19 | 50 | 2.2 | 1 |
| 183 | 0.21 | 50 | 0.70 | 0.40 |
| 184 | 0.021 | 50 | 0.010 | 0.0062 |
| 185 | 0.0061 | 50 | 0.0087 | 0.0017 |
| 186 | 0.0021 | 50 | 0.018 | 0.0074 |
| 187 | 0.0059 | 50 | 0.012 | 0.0043 |
| 188 | 0.00068 | 50 | | 0.0027 |
| 189 | 0.00030 | 9.8 | | 0.0056 |
| 190 | 0.000060 | 12 | | 0.0028 |
| 192 | 0.00021 | 50 | | 0.0053 |
| 193 | 0.00060 | 16 | | 0.0050 |
| 194 | 0.0015 | 50 | | 0.0048 |
| 195 | 0.0013 | 50 | 0.011 | 0.0071 |
| 196 | 0.00074 | 35 | 0.0067 | 0.0024 |
| 197 | 0.00074 | 19 | 0.0045 | 0.0021 |
| 198 | 0.0017 | 19 | 0.0080 | 0.0031 |
| 199 | 0.00098 | 21 | 0.0075 | 0.0030 |
| 200 | 0.00017 | 30 | 0.0069 | 0.0026 |
| 201 | 0.00054 | 22 | 0.0055 | 0.0022 |
| 202 | 0.00055 | 39 | 0.0073 | 0.0029 |
| 203 | 25 | 50 | 0.63 | 0.48 |
| 204 | 0.048 | 50 | | 0.16 |
| 205 | 0.50 | 25 | >10 | 2.5 |
| 206 | 0.50 | 25 | >10 | >10 |
| 207 | 3.5 | 50 | 10 | >10 |
| 208 | 0.66 | 50 | 5.6 | 3.2 |
| 209 | 0.099 | 50 | 1.2 | 0.24 |
| 210 | 0.28 | 25 | 0.39 | 0.092 |
| 211 | 0.27 | 25 | 0.43 | 0.074 |
| 212 | 0.088 | 38 | 0.56 | 0.29 |
| 213 | 0.14 | 30 | 0.34 | 0.22 |
| 214 | 0.016 | 50 | 0.012 | 0.0053 |
| 215 | 0.0055 | 50 | 0.0075 | 0.0022 |
| 216 | 0.071 | 50 | 0.57 | 0.34 |
| 217 | 0.0037 | 50 | 0.0061 | 0.0017 |
| 218 | 0.016 | 50 | 0.014 | 0.0048 |
| 219 | 0.0056 | 0.50 | | 0.0075 |
| 220 | 0.0026 | 38 | | 0.010 |
| 221 | 0.0015 | 18 | 0.011 | 0.0038 |
| 222 | 0.0021 | 50 | 0.013 | 0.0065 |
| 223 | 0.27 | 50 | | 1.2 |
| 224 | 0.00083 | 50 | 0.014 | 0.011 |
| 225 | 0.059 | 50 | 0.0060 | 0.0025 |
| 226 | 0.032 | 0.50 | 0.0082 | 0.0033 |
| 227 | 0.088 | 50 | | 0.15 |
| 228 | 0.060 | 0.50 | | 0.42 |
| 229 | 0.057 | 50 | | |
| 230 | 0.030 | 0.50 | | 0.28 |
| 231 | 0.0080 | 0.50 | | 0.0038 |
| 232 | 0.0064 | 50 | 0.0085 | 0.0033 |
| 233 | 0.0090 | 50 | 0.071 | 0.025 |
| 234 | 0.59 | 50 | | 0.21 |
| 235 | 0.23 | 0.50 | | 0.029 |
| 236 | 0.35 | 50 | | 0.019 |
| 237 | 0.00014 | 50 | | 0.0089 |
| 238 | 0.043 | 0.50 | | 0.40 |
| 239 | 0.073 | 0.50 | | 0.57 |
| 240 | 0.029 | 0.50 | | 0.51 |
| 241 | 0.00099 | 8.1 | | 0.019 |
| 242 | 0.0071 | 50 | | 0.021 |
| 243 | 0.0015 | 50 | | 0.011 |
| 244 | 0.0037 | 0.50 | | 0.0064 |
| 245 | 0.10 | 50 | | 0.72 |
| 246 | 0.00015 | 50 | | 0.0059 |
| 247 | 0.49 | 13 | | 6.0 |
| 248 | 5.1 | 50 | | >10 |
| 249 | 14 | 50 | | >10 |
| 250 | 50 | 50 | | >10 |
| 251 | 2.5 | 50 | | >10 |
| 252 | 50 | 50 | | >10 |
| 253 | 20 | 19 | | >10 |
| 254 | 10 | 50 | | >10 |
| 255 | 50 | 50 | | >10 |
| 256 | 0.0023 | 50 | 0.012 | 0.012 |
| 257 | 0.0014 | 50 | 0.010 | 0.0057 |
| 258 | 0.0023 | 50 | 0.013 | 0.0063 |
| 259 | 0.0086 | 50 | 0.012 | 0.0063 |
| 260 | 0.0028 | 50 | 0.0074 | 0.0017 |
| 261 | 0.0046 | 50 | | 0.017 |
| 262 | 0.000080 | 50 | | 0.0070 |
| 263 | 0.0027 | 50 | | 0.011 |
| 264 | 0.0023 | 33 | 0.020 | 0.0040 |
| 265 | 0.0035 | 50 | 0.0064 | 0.0048 |
| 266 | 0.0066 | 50 | 0.020 | 0.0068 |
| 267 | 0.0032 | 50 | 0.0099 | 0.0052 |
| 268 | 0.076 | 50 | 0.54 | 0.23 |
| 269 | 0.065 | 50 | 0.80 | 0.29 |
| 270 | 0.0089 | 50 | 0.16 | 0.28 |
| 271 | 0.0036 | 50 | 0.015 | 0.0059 |
| 272 | 0.20 | 50 | | 0.64 |
| 273 | 0.0019 | 50 | | 0.011 |
| 274 | 0.0028 | 38 | | 0.071 |
| 275 | 0.60 | 11 | | 0.30 |
| 276 | 0.027 | 32 | 0.18 | 0.089 |
| 277 | 0.040 | 31 | 0.26 | 0.15 |
| 278 | 0.090 | 50 | 0.45 | 0.17 |
| 279 | 0.064 | 50 | 0.29 | 0.17 |
| 280 | 0.023 | 50 | 0.22 | 0.094 |
| 281 | 0.043 | 50 | 0.33 | 0.28 |
| 282 | 0.041 | 8.0 | 0.31 | 0.16 |
| 283 | 0.44 | 20 | 10 | 4.3 |
| 284 | 0.10 | 6.8 | 0.65 | 0.22 |
| 285 | 0.092 | 16 | 0.63 | 0.38 |
| 286 | 0.028 | 50 | 8.6 | 4.4 |
| 287 | 0.069 | 50 | | 0.021 |
| 288 | 0.0014 | 20 | 0.0067 | 0.0028 |
| 289 | 0.0018 | 16 | 0.0087 | 0.0029 |
| 290 | 0.0047 | 16 | 0.0062 | 0.0017 |
| 291 | 0.0049 | 50 | | 0.0086 |
| 292 | 0.0016 | 10 | | 0.0088 |
| 293 | 0.0016 | 50 | | 0.0079 |
| 294 | 0.046 | 50 | | 0.15 |
| 295 | 0.0010 | 12 | | 0.0092 |
| 296 | 0.0013 | 35 | | 0.0083 |
| 297 | 0.0069 | 9.3 | 0.010 | 0.0076 |
| 298 | 0.0049 | 50 | 0.0061 | 0.0020 |
| 299 | 0.00090 | 50 | 0.0071 | 0.0020 |
| 300 | 0.018 | 0.50 | 0.094 | 0.045 |
| 301 | 0.028 | 0.50 | 0.13 | 0.080 |
| 302 | 0.042 | 0.50 | 0.17 | 0.069 |
| 303 | 0.014 | 0.50 | | 0.032 |
| 304 | 0.011 | 0.50 | | 0.047 |
| 305 | 0.0081 | 0.50 | | 0.065 |
| 306 | 0.012 | 0.50 | | 0.067 |
| 307 | 0.03 | 50 | | |
| 308 | 0.0054 | 11 | | 0.11 |
| 309 | 0.10 | 50 | | 0.045 |
| 310 | 0.015 | 13 | | 0.27 |
| 311 | 0.030 | 50 | | 0.041 |
| 312 | 0.024 | 0.50 | | 0.0041 |
| 313 | 0.0027 | 0.50 | | 0.010 |
| 314 | 0.0063 | 0.50 | | 0.022 |
| 315 | 0.0039 | 0.50 | | 0.014 |
| 316 | 0.0016 | 0.50 | | 0.0050 |
| 317 | 0.0014 | 0.50 | | 0.0087 |
| 318 | 0.0020 | 0.50 | | 0.0087 |
| 319 | 50 | 50 | | >10 |
| 320 | 0.027 | 50 | | 0.0094 |
| 321 | 0.0060 | 24 | | 0.052 |
| 322 | 0.0014 | 21 | | 0.012 |
| 323 | 0.097 | 50 | | 0.93 |
| 324 | 0.0030 | 0.50 | | 0.019 |
| 325 | 0.023 | 50 | | 0.10 |
| 326 | 0.060 | 50 | | 0.18 |
| 327 | 0.0070 | 31 | | 0.029 |
| 328 | 0.050 | 27 | | 0.075 |
| 329 | 0.26 | 40 | | 0.16 |
| 330 | 0.0050 | 30 | | 0.011 |
| 331 | 0.0065 | 30 | | 0.076 |
| 332 | 0.57 | 35 | | 4.7 |
| 333 | 0.055 | 30 | | 0.67 |
| 334 | 0.0095 | 50 | 0.012 | 0.0049 |
| 335 | 0.65 | 50 | | >10 |
| 336 | 0.054 | 50 | | 0.87 |
| 337 | 1.1 | 50 | | >10 |
| 338 | 1.9 | 18 | | >10 |
| 339 | 3.1 | 50 | | >10 |
| 340 | 0.077 | 25 | | 0.85 |
| 341 | 2.9 | 50 | | >10 |
| 342 | 0.84 | 35 | | 1.3 |
| 343 | 0.0023 | 24 | | 0.016 |
| 344 | 0.0040 | 50 | | 0.021 |
| 345 | 0.017 | 8.2 | | 0.044 |
| 346 | 0.059 | 50 | | 0.53 |
| 347 | 2.4 | 50 | | 4.0 |
| 348 | 0.0018 | 50 | | 0.0092 |
| 349 | 0.014 | 50 | | 0.043 |
| 350 | 0.0012 | 50 | | 0.020 |
| 351 | 6.7 | 50 | 0.15 | 0.056 |
| 352 | 7.5 | 50 | 0.35 | 0.12 |
| 353 | 0.0072 | 28 | | 0.027 |
| 354 | 0.015 | 19 | | 0.026 |
| 355 | 0.0034 | 50 | | 0.0084 |
| 356 | 0.026 | 50 | | |
| 357 | 0.0053 | 50 | | |
| 358 | 0.0057 | 50 | | 0.041 |
| 359 | 4.0 | 50 | >10 | >10 |
| 360 | 10 | 50 | 2.5 | 1.7 |
| 361 | 0.56 | 50 | | 3.9 |
| 362 | 0.0039 | 31 | | 0.033 |
| 363 | 0.021 | 50 | 0.057 | 0.019 |
| 364 | 0.17 | 50 | | |
| 365 | 21 | 35 | >10 | >10 |
| 366 | 25 | 50 | >10 | >10 |

1: A compound of formula (I): 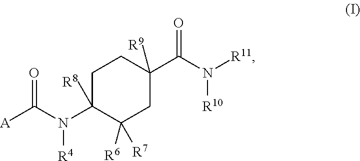
in which,
A represents a group selected from: 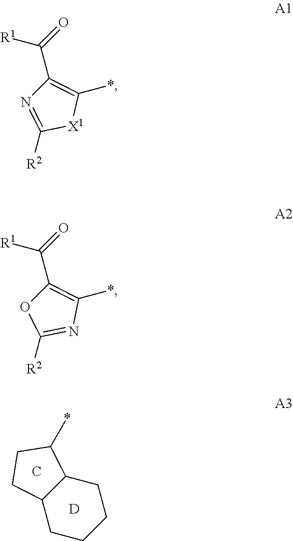
wherein * indicates the point of attachment of said groups with the rest of the molecule, wherein said point of attachment is a carbon atom; 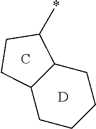
represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by one or two N atoms, said ring C being optionally substituted with one or two R⁵ groups, and
ring D represents a phenyl group or a 6-membered heteroaryl group which contains one, two or three nitrogen heteroatoms, said ring D being optionally substituted with one, two or three R¹² groups;
X¹ represents NR³ or O,
R¹ represents a group selected from:
—OR³, and —N(R¹⁴)R¹⁵,
R² represents a group selected from:
hydrogen, C₁-C₃-alkyl, and C₃-C₄-cycloalkyl,
R³ represents a hydrogen atom,
R⁴ represents a hydrogen atom,
R⁵ represents, independently of each other, a group selected from:
halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, and —C(O)OR¹³,
wherein C₁-C₄-alkyl is optionally substituted one, two or three times with a group independently selected from halogen, hydroxy, C₁-C₃-alkoxy, —NH₂, —NH(C₁-C₃-alkyl) and —N(C₁-C₃-alkyl)₂;
R⁶ represents hydrogen, halogen, hydroxy, C₁-C₃-alkyl or C₁-C₃-alkoxy;
R⁷ represents hydrogen; or
R⁶, R⁷ represent, independently of each other, halogen;
in embodiment a), R⁸, R⁹ and R¹⁰ represent:
R⁸ represents hydrogen, or C₁-C₃-alkyl,
R⁹ represents hydrogen, halogen, C₁-C₃-alkoxy, or C₁-C₃-alkyl optionally substituted with one, two or three groups independently selected from hydroxy, halogen and C₃-C₄-cycloalkyl; or
R⁸ and R⁹ together represent a group: 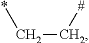
wherein * indicates the point of attachment of said group to the rest of the molecule at R⁸,
and ^{#} indicates the point of attachment of said group to the rest of the molecule at R⁹; and
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-(C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-; or
in embodiment b), R⁸, R⁹ and R¹⁰ represent:
R⁸ represents hydrogen, or C₁-C₃-alkyl, and
R⁹ and R¹⁰ together represent a group selected from: 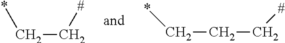
wherein said groups are optionally substituted with one or two groups, which are independently of each other selected from:
halogen, C₁-C₃-alkyl and C₁-C₃-alkoxy, hydroxy, C₁-C₃-haloalkyl, C₁-C₃-hydroxyalkyl,
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
wherein aryl and heteroaryl groups are optionally substituted with one, two, three or four groups, which are independently of each other selected from:
C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
R¹² represents, independently of each other, halogen, hydroxy, C₁-C₆-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
wherein C₁-C₆-alkyl is optionally substituted one, two or three times with a substituent independently selected from halogen, hydroxy, C₁-C₃-alkoxy, C₁-C₃-haloalkoxy, and —N(R¹⁸)R¹⁹;
whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl;
R¹³ represents a group selected from:
C₁-C₆-alkyl, C₃-C₆-cycloalkyl, C₂-C₆-hydroxyalkyl-, and (C₁-C₃-alkoxy)-(C₂-C₆-alkyl)-,
R¹⁴ and R¹⁵ are independently of each other selected from:
hydrogen, C₁-C₆-alkyl, C₃-C₆-cycloalkyl, (C₃-C₆-cycloalkyl)-(C₁-C₆-alkyl)-, C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
wherein 4- to 6-membered heterocycloalkyl groups are optionally substituted with one, two, three or four substituents, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, —NH(C₁-C₃-alkyl), —N(C₁-C₃-alkyl)₂, hydroxy, a halogen atom, and cyano,
and,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
said 4- to 7-membered heterocycloalkyl group being optionally substituted with one, two, three or four groups, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, NH₂, N(CH₃)H, hydroxy, a halogen atom, and cyano,
whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
said azetidine and pyrrolidine being optionally substituted one time with C₁-C₃-alkyl or C₁-C₃-haloalkyl,
or,
R¹⁴ and R¹⁵ together with the nitrogen atom to which they are attached form a group selected from: 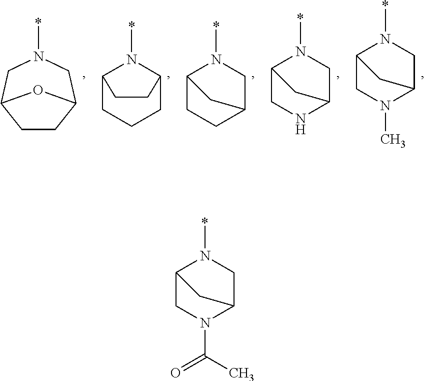
wherein * indicates the point of attachment of said group with the rest of the molecule,
R¹⁶ represents, independently of each other, hydrogen, or C₁-C₃-alkyl,
R¹⁷ represents hydrogen, C₁-C₆-alkyl, C₁-C₆-hydroxyalkyl, C₃-C₆-cycloalkyl,
C₁-C₆-haloalkyl, (C₁-C₃-alkoxy)-(C₁-C₆-alkyl)-, aryl, or heteroaryl,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy,
R¹⁸ and R¹⁹ are, independently of each other, selected from:
hydrogen, C₁-C₆-alkyl, C₃-C₆-cycloalkyl, (C₃-C₆-cycloalkyl)-(C₁-C₆-alkyl)-, C₂-C₆-hydroxyalkyl-, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, and heteroaryl,
wherein 4- to 6-membered heterocycloalkyl groups are optionally substituted with one two, three or four substituents, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
and,
wherein aryl and heteroaryl groups are optionally substituted with one or two substituents, which are independently of each other selected from:
C₁-C₃-alkyl, halogen, cyano, -
or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O), said 4- to 7-membered heterocycloalkyl group being optionally substituted with one, two, three or four groups, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group,
said azetidine and pyrrolidine being optionally substituted one time with C₁-C₃-alkyl or C₁-C₃-haloalkyl;
R²⁰ represents, independently of each other, a group selected from:
hydrogen, C₁-C₃-alkyl, C₁-C₃-haloalkyl, and C₃-C₄-cycloalkyl,
R²¹ represents hydrogen, cyano, (C₁-C₃-alkyl)-C(═O)—, or (C₁-C₃-haloalkyl)-C(═O)—,
R²² represents C₁-C₄-alkyl, or C₃-C₄-cycloalkyl,
or a stereoisomer, a tautomer, an N-oxide, a hydrate, a solvate, or a salt thereof, or a mixture of same.

2: The compound of formula (I) according to claim 1, wherein:
A represents a group selected from: 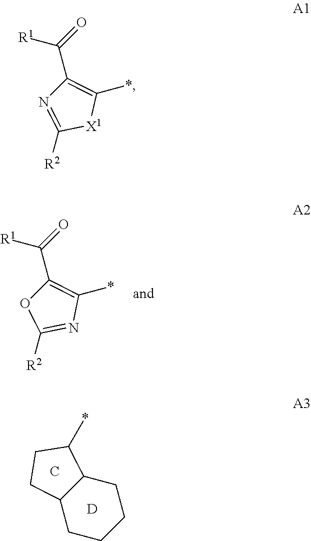
wherein * indicates the point of attachment of said groups with the rest of the molecule, wherein said point of attachment is a carbon atom; 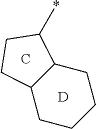
represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by a N atom, said ring C being optionally substituted with one or two R⁵ groups, and
ring D represents a phenyl group or a 6-membered heteroaryl group which contains one, two or three nitrogen heteroatoms, said ring D being optionally substituted with one, two or three R¹² groups;
X¹ represents NR³ or O,
R¹ represents a group selected from:
—OR³, and —N(R¹⁴)R¹⁵,
R² represents a group selected from:
hydrogen, and C₁-C₃-alkyl,
R³ represents a hydrogen atom,
R⁴ represents a hydrogen atom,
R⁵ represents, independently of each other, a group selected from:
halogen, hydroxy, C₁-C₃-alkyl, and —NH₂,
wherein C₁-C₃-alkyl is optionally substituted one, two or three times with a group independently selected from halogen, hydroxy, C₁-C₃-alkoxy, —NH₂, —NH(C₁-C₃-alkyl) and —N(C₁-C₃-alkyl)₂;
R⁶ represents hydrogen, halogen, hydroxy, C₁-alkyl or C₁-alkoxy;
R⁷ represents hydrogen; or
R⁶, R⁷ represent, independently of each other, halogen;
in embodiment a), R⁸, R⁹ and R¹⁰ represent:
R⁸ represents hydrogen, or C₁-C₃-alkyl,
R⁹ represents hydrogen, halogen, C₁-alkoxy or C₁-C₃-alkyl optionally substituted with one, two or three groups independently selected from hydroxy and halogen; or
R⁸ and R⁹ together represent a group: 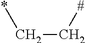
wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-; or
in embodiment b), R⁸, R⁹ and R¹⁰ represent:
R⁸ represents hydrogen, or C₁-C₃-alkyl,
R⁹ and R¹⁰ together represent a group selected from: 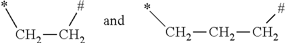
wherein said groups are optionally substituted with one or two groups, which are independently of each other selected from:
halogen, C₁-C₃-alkyl and C₁-C₃-alkoxy, hydroxy, C₁-C₃-haloalkyl, and C₁-C₃-hydroxyalkyl;
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
wherein aryl and heteroaryl groups are optionally substituted with one, two, three or four groups, which are independently of each other selected from:
C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
R¹² represents, independently of each other, halogen, hydroxy, C₁-C₆-alkyl, C₃-C₆-cycloalkyl, C₁-C₄-alkoxy, C₁-C₄-haloalkoxy, —N(R¹⁸)R¹⁹, —C(O)R¹³, or —C(O)OR¹³,
wherein C₁-C₆-alkyl is optionally substituted one, two or three times with a substituent independently selected from halogen, hydroxy, C₁-C₃-alkoxy, C₁-C₃-haloalkoxy, —N(R¹⁸)R¹⁹;
whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl;
R¹³ represents a group selected from:
C₁-C₆-alkyl, C₃-C₆-cycloalkyl, C₂-C₆-hydroxyalkyl-, and (C₁-C₃-alkoxy)-(C₂-C₆-alkyl)-,
R¹⁴ and R¹⁵ are independently of each other selected from:
hydrogen, C₁-C₆-alkyl, C₃-C₆-cycloalkyl, (C₃-C₆-cycloalkyl)-(C₁-C₆-alkyl)-, C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
wherein 4- to 6-membered heterocycloalkyl groups are optionally substituted with one, two, three or four substituents, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
and,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O), said 4- to 7-membered heterocycloalkyl group being optionally substituted with one, two, three or four groups, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, —N(H)₂, N(CH₃)H, hydroxy, a halogen atom, and cyano,
whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group,
said azetidine and pyrrolidine being optionally substituted one time with C₁-C₃-alkyl or C₁-C₃-haloalkyl,
R¹⁶ represents, independently of each other, hydrogen, or C₁-C₃-alkyl,
R¹⁷ represents hydrogen, C₁-C₆-alkyl, C₁-C₆-hydroxyalkyl, C₃-C₆-cycloalkyl, C₁-C₆-haloalkyl, (C₁-C₃-alkoxy)-(C₁-C₆-alkyl)-, aryl, or heteroaryl,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy,
R¹⁸ and R¹⁹ are, independently of each other, selected from:
hydrogen, C₁-C₆-alkyl, C₃-C₆-cycloalkyl, (C₃-C₆-cycloalkyl)-(C₁-C₆-alkyl)-, C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, and (4- to 6-membered heterocycloalkyl)-(C₁-C₆-alkyl)-,
wherein 4- to 6-membered heterocycloalkyl groups are optionally substituted with one, two, three or four substituents, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O), said 4- to 7-membered heterocycloalkyl group being optionally substituted with one, two, three or four groups, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, NH₂, N(CH₃)H, hydroxy, a halogen atom, and cyano,
R²⁰ represents, independently of each other, a group selected from:
hydrogen, C₁-C₃-alkyl, C₁-C₃-haloalkyl, and C₃-C₄-cycloalkyl,
R²¹ represents hydrogen, cyano, (C₁-C₃-alkyl)-C(═O)—, or (C₁-C₃-haloalkyl)-C(═O)—,
R²² represents C₁-C₄-alkyl, or C₃-C₄-cycloalkyl,
or a stereoisomer, a tautomer, an N-oxide, a hydrate, a solvate, or a salt thereof, or a mixture of same.

3: The compound of formula (I) according to claim 1, wherein:
A represents a group selected from: 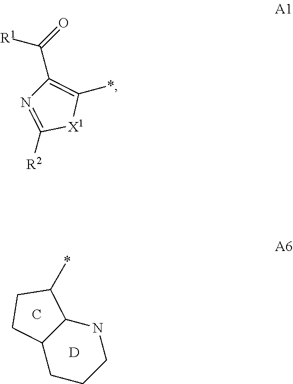
wherein * indicates the point of attachment of said groups with the rest of the molecule, wherein said point of attachment is a carbon atom; 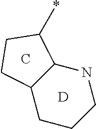
represents a bicyclic aromatic ring system, wherein ring C represents a 5-membered heteroaryl group which contains one heteroatom-containing group selected from N, NH, and N(C₁-C₃-alkyl), in which one or two carbon atoms are optionally further replaced by a N atom, said ring C being optionally substituted with one or two R⁵ groups, and
ring D represents a 6-membered heteroaryl group which contains one, two or three nitrogen heteroatoms, said ring D being optionally substituted with one, two or three R¹² groups;
X¹ represents NR³ or O,
R¹ represents a group selected from:
OR³, and —N(R¹⁴)R¹⁵,
R² represents a group selected from:
hydrogen, and C₁-C₂-alkyl,
R³ represents a hydrogen atom,
R⁴ represents a hydrogen atom,
R⁵ represents, independently of each other, a group selected from:
halogen, hydroxy, C₁-C₂-alkyl, and —NH₂,
wherein C₁-C₂-alkyl is optionally substituted one, two or three times, independently of each other, with halogen or optionally substituted one time with a group selected from hydroxy and C₁-alkoxy;
R⁶ represents hydrogen, fluorine, hydroxy, C₁-alkyl or C₁-alkoxy;
R⁷ represents hydrogen; or
R⁶, R⁷ represent fluorine;
in embodiment a), R⁸, R⁹ and R¹⁰ represent:
R⁸ represents hydrogen, or C₁-C₃-alkyl,
R⁹ represents hydrogen, halogen, or C₁-C₃-alkyl; or
R⁸ and R⁹ together represent a group: 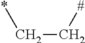
wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, C₃-C₄-cycloalkyl, (C₃-C₄-cycloalkyl)-(C₁-C₃-alkyl)-, C₂-C₃-hydroxyalkyl, (C₁-alkoxy)-(C₂-C₃-alkyl)-(C₁-haloalkoxy)-(C₂-C₃-alkyl)-, C₁-C₃-haloalkyl, H₂N—(C₂-C₃-alkyl)-, (C₁-alkyl)N(H)(C₂-C₃-alkyl)-, or (C₁-alkyl)₂N(C₂-C₃-alkyl)-; or
in embodiment b), R⁸, R⁹ and R¹⁰ represent:
R⁸ represents hydrogen, or C₁-C₃-alkyl,
R⁹ and R¹⁰ together represent a group selected from: 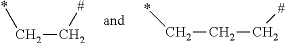
wherein said groups are optionally substituted with one or two groups, which are independently of each other selected from:
halogen, C₁-C₃-alkyl and C₁-C₃-alkoxy, hydroxy, C₁-C₃-haloalkyl, C₁-C₃-hydroxyalkyl,
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
wherein aryl and heteroaryl groups are optionally substituted with one, two, three or four groups, which are independently of each other selected from:
C₁-C₆-alkyl, C₁-C₃-alkoxy, C₁-C₃-hydroxyalkyl, C₃-C₆-cycloalkyl, C₃-C₆-cycloalkoxy, C₁-C₃-haloalkyl, C₁-C₃-haloalkoxy, halogen, cyano, nitro, hydroxy, (C₁-C₆-alkyl)-S—, (C₁-C₆-alkyl)-S(═O)—, (C₁-C₆-alkyl)-S(═O)₂—, —S(═O)(═NR²¹)R²², —N(R¹⁴)R¹⁵, R¹⁴(R¹⁵)N—(C₁-C₆-alkyl)-, R¹⁴(R¹⁵)N—(C₂-C₆-alkoxy)-, phenyl, phenoxy, —N(R¹⁶)C(═O)R¹⁷, —C(═O)OH, —C(═O)OR¹³, and —C(═O)N(R¹⁶)₂,
whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
R¹² represents, independently of each other, halogen, hydroxy, C₁-C₆-alkyl, C₃-C₆-cycloalkyl, C₁-C₄-alkoxy, C₁-C₄-haloalkoxy, —N(R⁸)R¹⁹, —C(O)R¹³, or —C(O)OR¹³,
wherein C₁-C₆-alkyl is optionally substituted one, two or three times with a substituent independently selected from halogen, hydroxy, C₁-C₃-alkoxy, C₁-C₃-haloalkoxy, and —N(R¹⁸)R¹⁹;
whereby two substituents R¹² when they are in adjacent positions of the ring to which they are attached, can be linked to one another in such a way that they jointly form methanediylbisoxy, ethane-1,2-diylbisoxy, propane-1,3-diyl, or butane-1,4-diyl;
R¹³ represents a group selected from:
C₁-C₃-alkyl, C₃-C₄-cycloalkyl, C₂-C₃-hydroxyalkyl-, and (C₁-alkoxy)-(C₂-C₃-alkyl)-,
R¹⁴ and R¹⁵ are independently of each other selected from:
hydrogen, C₁-C₆-alkyl, C₃-C₆-cycloalkyl, (C₃-C₆-cycloalkyl)-(C₁-C₆-alkyl)-, C₂-C₆-hydroxyalkyl, (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, (4- to 6-membered heterocycloalkyl)-(C₁-C₆-alkyl)-, aryl, heteroaryl, aryl-(C₁-C₆-alkyl)-, and heteroaryl-(C₁-C₆-alkyl)-,
wherein 4- to 6-membered heterocycloalkyl groups are optionally substituted with one or two substituents, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
and,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, —C(═O)OH, —C(═O)OR¹³, and —C(═O)N(R¹⁶)₂,
or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, and in which one additional ring atom is optionally replaced by C(═O),
said 4- to 7-membered heterocycloalkyl group being optionally substituted with one, two, three or four groups, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, —NH₂, —N(CH₃)H, hydroxy, a halogen atom, and cyano,
whereby when two substituents are attached to the same ring carbon atom, together with the carbon atom to which they are attached, can be linked to one another in such a way that they jointly form a cyclobutane, cyclopentane, azetidine, pyrrolidine, oxetane, tetrahydrofuran, thietane, tetrahydrothiophene, thietane 1-oxide, tetrahydrothiophene 1-oxide, thietane 1,1-dioxide or tetrahydrothiophene 1,1-dioxide group;
said azetidine and pyrrolidine being optionally substituted one time with C₁-C₃-alkyl or C₁-C₃-haloalkyl,
R¹⁶ represents, independently of each other, hydrogen, or C₁-C₃-alkyl,
R¹⁷ represents hydrogen, C₁-C₆-alkyl, C₁-C₆-hydroxyalkyl, C₃-C₆-cycloalkyl, C₁-C₆-haloalkyl, (C₁-C₃-alkoxy)-(C₁-C₆-alkyl)-, aryl, or heteroaryl,
wherein aryl and heteroaryl groups are optionally substituted with one or two substituents, which are independently of each other selected from:
C₁-C₃-alkyl, C₃-C₆-cycloalkyl, C₁-C₃-alkoxy, C₃-C₆-cycloalkoxy, C₁-C₃-haloalkyl, C₁-C₃-haloalkoxy, halogen, cyano, and hydroxy,
R¹⁸ and R¹⁹ are, independently of each other, selected from:
hydrogen, C₁-C₆-alkyl, C₃-C₆-cycloalkyl, (C₃-C₆-cycloalkyl)-(C₁-C₆-alkyl)-, C₂-C₆-hydroxyalkyl (C₁-C₃-alkoxy)-(C₂-C₆-alkyl)-, C₁-C₆-haloalkyl, H₂N—(C₂-C₆-alkyl)-, (C₁-C₃-alkyl)N(H)(C₂-C₆-alkyl)-, (C₁-C₃-alkyl)₂N(C₂-C₆-alkyl)-, HOC(═O)—(C₁-C₆-alkyl)-, R¹³OC(═O)—(C₁-C₆-alkyl)-, 4- to 6-membered heterocycloalkyl, and (4- to 6-membered heterocycloalkyl)-(C₁-C₆-alkyl)-,
wherein 4- to 6-membered heterocycloalkyl groups are optionally substituted with one or two substituents, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —NH₂, hydroxy, a halogen atom, and cyano,
or,
R¹⁸ and R¹⁹ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, in which heterocycloalkyl group one additional ring atom is optionally replaced by C(═O), said 4- to 7-membered heterocycloalkyl group being optionally substituted with one, two, three or four groups, which are independently of each other selected from:
C₁-C₃-alkyl, C₁-C₃-haloalkyl, C₁-C₃-alkoxy, C₁-C₃-haloalkoxy, C₃-C₄-cycloalkyl, C₃-C₄-cycloalkoxy, —N(CH₃)₂, N(H)₂, N(CH₃)H, hydroxy, a halogen atom, and cyano,
R²⁰ represents, independently of each other, a group selected from:
hydrogen, C₁-C₃-alkyl, C₁-C₃-haloalkyl, and C₃-C₄-cycloalkyl,
R²¹ represents hydrogen, cyano, (C₁-C₃-alkyl)-C(═O)—, or (C₁-C₃-haloalkyl)-C(═O)—,
R²² represents C₁-C₄-alkyl, or C₃-C₄-cycloalkyl,
or a stereoisomer, a tautomer, an N-oxide, a hydrate, a solvate, or a salt thereof, or a mixture of same.

4: The compound according to claim 1, wherein:
A represents a group selected from: 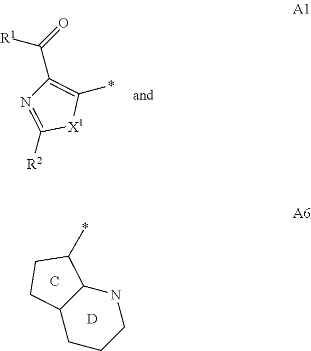
wherein * indicates the point of attachment of said groups with the rest of the molecule, wherein said point of attachment is a carbon atom; 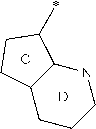
represents a bicyclic aromatic ring system, wherein ring C represents a 5-membered heteroaryl group which contains one heteroatom-containing group selected from N, and NH, in which one or two carbon atoms are optionally further replaced by a nitrogen atom, said 5-membered ring being optionally substituted with one or two R⁵ groups, and
ring D represents a 6-membered heteroaryl group which contains one, two or three nitrogen heteroatoms, said ring being optionally substituted with one, two or three R¹² groups,
X¹ represents NR³ or O,
R¹ represents a group selected from:
—OR³, and —N(R¹⁴)R¹⁵,
R² represents a group selected from:
hydrogen, C₁-C₂-alkyl,
R³ represents a hydrogen atom,
R⁴ represents a hydrogen atom,
R⁵ represents, independently of each other, a group selected from:
halogen, hydroxy, C₁-C₂-alkyl, and —NH₂,
wherein C₁-C₂-alkyl is optionally substituted one time with a group selected from hydroxy and C₁-alkoxy;
R⁶ represents hydrogen, fluorine, C₁-alkyl or C₁-alkoxy;
R⁷ represents hydrogen; or
R⁶, R⁷ represent fluorine;
in embodiment a), R⁸, R⁹ and R¹⁰ represent:
R⁸ represents hydrogen,
R⁹ represents hydrogen, fluorine or C₁-alkyl; or
R⁸ and R⁹ together represent a group: 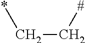
wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, C₂-C₃-hydroxyalkyl, (C₁-alkoxy)-(C₂-C₃-alkyl)-, (C₁-haloalkoxy)-(C₂-C₃-alkyl)-, or C₁-C₃-haloalkyl; or
in embodiment b), R⁸, R⁹ and R¹⁰ represent:
R⁸ represents hydrogen,
R⁹ and R¹⁰ together represent a group selected from: 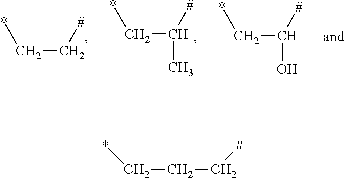
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
wherein aryl and heteroaryl groups are optionally substituted with one, two, three or four groups, which are independently of each other selected from:
C₁-C₄-alkyl, C₁-C₃-alkoxy, C₁-C₃-hydroxyalkyl, C₃-C₄-cycloalkyl, C₃-C₄-cycloalkoxy, C₁-C₃-haloalkyl, C₁-C₃-haloalkoxy, halogen, cyano, hydroxy, —C(═O)OR¹³, —N(R¹⁴)R¹⁵, R¹⁴(R¹⁵)N—(C₁-C₃-alkyl)- and R¹⁴(R¹⁵)N—(C₂-C₃-alkoxy)-,
whereby two substituents of said aryl group, when they are in ortho-position to one another, can be linked to one another in such a way that they jointly form methanediylbisoxy, ethane-1,2-diylbisoxy, propane-1,3-diyl, or butane-1,4-diyl,
R¹² represents, independently of each other, halogen, hydroxy, C₁-C₄-alkyl, C₃-C₄-cycloalkyl, C₁-C₃-alkoxy, C₁-C₃-haloalkoxy, —N(R¹⁸)R¹⁹, —C(O)R¹³, or —C(O)OR¹³,
wherein C₁-C₄-alkyl is optionally substituted one, two or three times with halogen and optionally substituted one time with a substituent independently selected from hydroxy, C₁-C₃-alkoxy, C₁-C₃-haloalkoxy, —N(R¹⁸)R¹⁹;
whereby two substituents R¹² when they are in adjacent positions of the ring to which they are attached, can be linked to one another in such a way that they jointly form propane-1,3-diyl;
R¹³ represents C₁-C₂-alkyl,
R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-, C₂-C₄-hydroxyalkyl, (C₁-alkoxy)-(C₂-C₄-alkyl)-, C₁-C₃-haloalkyl, H₂N—(C₂-C₃-alkyl)-, (C₁-C₃-alkyl)N(H)(C₂-C₃-alkyl)-, (C₁-C₃-alkyl)₂N(C₂-C₃-alkyl)-, 4- to 6-membered heterocycloalkyl, (4- to 6-membered heterocycloalkyl)-(C₁-C₃-alkyl)-,
wherein 4- to 6-membered heterocycloalkyl groups are optionally substituted with one or two substituents, which are independently of each other selected from:
C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, said 4- to 7-membered heterocycloalkyl group being optionally substituted with one or two groups, which are independently of each other selected from:
C₁-alkyl, C₁-haloalkyl, C₁-alkoxy, C₁-haloalkoxy, C₃-C₄-cycloalkyl, C₃-C₄-cycloalkoxy, —N(CH₃)₂, —N(H)₂, —N(CH₃)H, hydroxy, and a halogen atom,
whereby when two substituents are attached to the same ring carbon atom, together with the carbon atom to which they are attached, can be linked to one another in such a way that they jointly form a azetidine, or oxetane;
said azetidine being optionally substituted one time with C₁-alkyl or C₁-haloalkyl,
R¹⁸ and R¹⁹ are, independently of each other, selected from:
hydrogen, C₁-C₄-alkyl, C₃-C₄-cycloalkyl, (C₃-C₄-cycloalkyl)-(C₁-C₃-alkyl)-, C₂-C₄-hydroxyalkyl, (C₁-alkoxy)-(C₂-C₃-alkyl)-, C₁-C₃-haloalkyl, H₂N—(C₂-C₃-alkyl)-, (C₁-alkyl)N(H)(C₂-C₃-alkyl)-, (C₁-alkyl)₂N(C₂-C₃-alkyl)-, HOC(═O)—(C₁-C₃-alkyl)-, R¹³OC(═O)—(C₁-C₃-alkyl)-, 4- to 6-membered heterocycloalkyl, and (4- to 6-membered heterocycloalkyl)-(C₁-C₃-alkyl)-,
wherein 4- to 6-membered heterocycloalkyl groups are optionally substituted with one or two substituents, which are independently of each other selected from:
C₁-alkyl, C₁-haloalkyl, C₁-alkoxy, C₁-haloalkoxy, —NH₂, hydroxy, and a halogen atom,
or,
R¹⁸ and R¹⁹ together with the nitrogen atom to which they are attached form a 5-6-membered heterocycloalkyl group, in which one carbon atom is optionally replaced by a further heteroatom-containing group selected from NR²⁰, O,
said 5-6-membered heterocycloalkyl group being optionally substituted with one or two groups, which are independently of each other selected from:
C₁-alkyl, C₁-haloalkyl, C₁-alkoxy, C₁-haloalkoxy, —NH₂, —N(CH₃)₂, N(CH₃)H, hydroxy, and a halogen atom,
R²⁰ represents, independently of each other, a group selected from:
hydrogen, C₁-alkyl, and C₁-C₂-haloalkyl,
or a stereoisomer, a tautomer, an N-oxide, a hydrate, a solvate, or a salt thereof, or a mixture of same.

5: The compound according to claim 1, wherein:
A represents a group selected from: 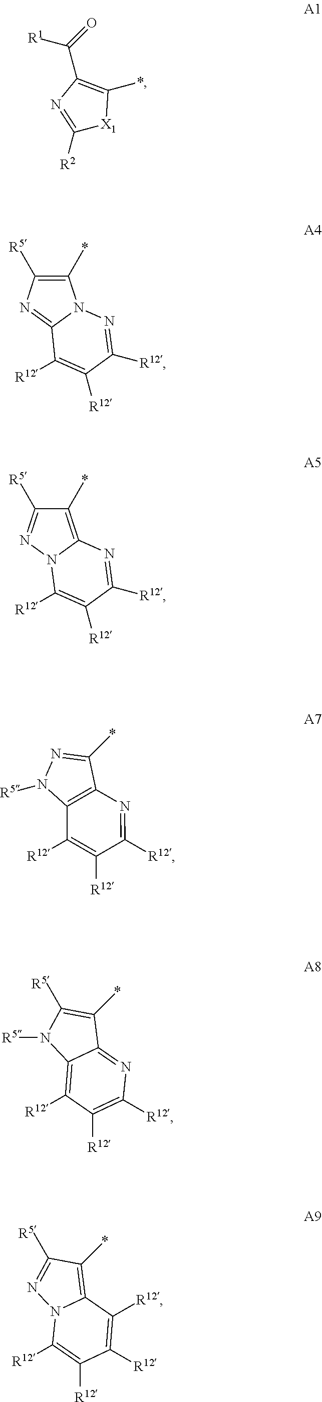 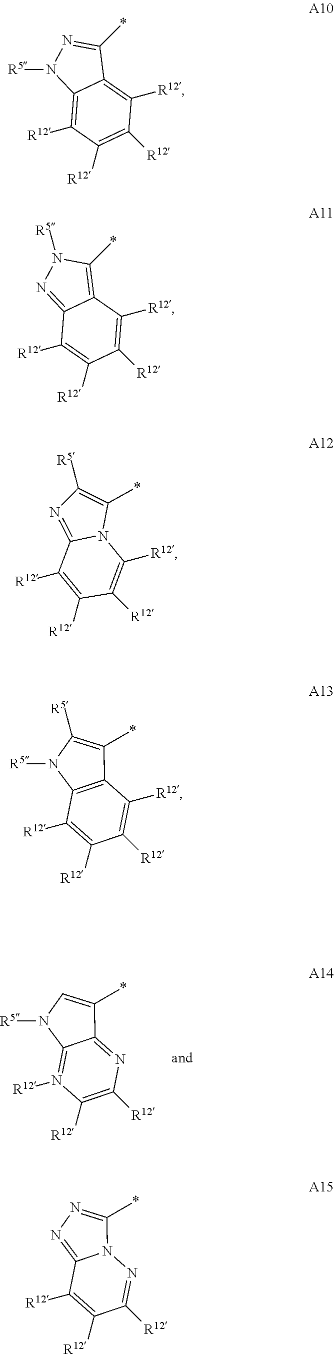
wherein * indicates the point of attachment of said groups with the rest of the molecule,
X¹ represents NR³ or O,
R¹ represents a group selected from:
—OR³, and —N(R¹⁴)R¹⁵,
R² represents hydrogen,
R³ represents a hydrogen atom,
R⁴ represents a hydrogen atom,
R^{5′} represents, independently of each other, a group selected from:
hydrogen, halogen, hydroxy, C₁-alkyl and —NH₂,
R^{5″} represents, independently of each other, a group selected from:
hydrogen, C₁-C₃-alkyl, C₂-hydroxyalkyl and (C₁-alkoxy)-(C₂-alkyl)-,
R⁶ represents hydrogen, fluorine, C₁-alkyl or C₁-alkoxy;
R⁷ represents hydrogen; or
R⁶, R⁷ represent fluorine;
in embodiment a), R⁸, R⁹ and R¹⁰ represent:
R⁸ represents hydrogen,
R⁹ represents hydrogen, or C₁-alkyl; or
R⁸ and R⁹ together represent a group: 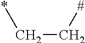
wherein * indicates the point of attachment of said group to the rest of the molecule at R⁸,
and ^{#} indicates the point of attachment of said group to the rest of the molecule at R⁹;
R¹⁰ represents hydrogen, C₁-C₃-alkyl, (C₃-C₄-cycloalkyl)-(C₁-C₃-alkyl)-, or C₂-C₃-hydroxyalkyl;
or
in embodiment b), R⁸, R⁹ and R¹⁰ represent:
R⁸ represents hydrogen,
R⁹ and R¹⁰ together represent a group selected from: 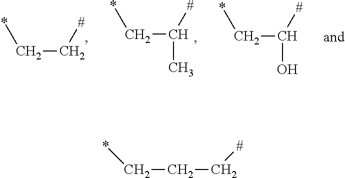
wherein * indicates the point of attachment of said group to the rest of the molecule at R⁹,
and ^{#} indicates the point of attachment of said group to the rest of the molecule at R¹⁰;
R¹¹ represents a group selected from:
aryl, and heteroaryl,
wherein aryl and heteroaryl groups are optionally substituted with one, two, three or four groups, which are independently of each other selected from:
C₁-C₄-alkyl, C₁-C₃-alkoxy, C₁-C₃-hydroxyalkyl, C₁-C₃-haloalkyl, C₁-C₃-haloalkoxy, halogen, cyano, —C(═O)OR¹³, and —N(R¹⁴)R¹⁵;
R^{12′} represents, independently of each other, hydrogen, halogen, hydroxy, C₁-C₄-alkyl, C₃-C₄-cycloalkyl, C₁-alkoxy, —N(R⁸)R¹⁹, —C(O)R¹³, or —C(O)OR¹³,
wherein C₁-C₄-alkyl is optionally substituted one, two or three times, independently of each other, with halogen and optionally substituted one time with a substituent selected from hydroxy, C₁-C₃-alkoxy, —NH₂, —NH(CH₃), —N(CH₃)₂,
whereby two substituents R^{12′} when they are in adjacent positions of the ring to which they are attached, can be linked to one another in such a way that they jointly form propane-1,3-diyl;
R¹³ represents C₁-C₂-alkyl,
R¹⁴ and R¹⁵ are independently of each other selected from:
hydrogen, C₁-C₃-alkyl, C₃-C₄-cycloalkyl, (C₃-C₄-cycloalkyl)-(C₁-C₃-alkyl)-, C₂-C₄-hydroxyalkyl, (C₁-alkoxy)-(C₂-C₄-alkyl)-, C₁-C₃-haloalkyl, (C₁-alkyl)₂N(C₂-C₃-alkyl)-, (C₁-C₃-alkyl)HN(C₂-C₃-alkyl)-, 4- to 6-membered heterocycloalkyl and (4- to 6-membered heterocycloalkyl)-(C₁-C₃-alkyl)-,
wherein 4- to 6-membered heterocycloalkyl groups are optionally substituted with one or two substituents, which are independently of each other selected from:
C₁-alkyl, C₁-haloalkyl, C₁-alkoxy, C₁-haloalkoxy, C₃-C₄-cycloalkyl, C₃-C₄-cycloalkoxy, —NH₂, hydroxy, and a halogen atom,
or,
R¹⁴ and R¹⁵ together with the nitrogen atom to which they are attached form a 4- to 7-membered heterocycloalkyl group, in which one carbon atom is optionally replaced by a further heteroatom-containing group selected from NR²⁰, O, S, S(═O) and S(═O)₂, said 4- to 7-membered heterocycloalkyl group being optionally substituted with one or two groups, which are independently of each other selected from:
C₁-alkyl, C₁-haloalkyl, C₁-alkoxy, C₁-haloalkoxy, —N(CH₃)₂, hydroxy and a halogen atom,
whereby when two substituents are attached to the same ring carbon atom, together with the carbon atom to which they are attached, can be linked to one another in such a way that they jointly form a azetidine, thietane 1,1-dioxide, or oxetane;
said azetidine being optionally substituted one time with C₁-alkyl or C₁-haloalkyl,
R¹⁸ and R¹⁹ are, independently of each other, selected from:
hydrogen and C₁-alkyl,
or,
R¹⁸ and R¹⁹ together with the nitrogen atom to which they are attached form a 5-6-membered heterocycloalkyl group, in which one carbon atom is optionally replaced by a further heteroatom-containing group selected from NR²⁰, and O,
R²⁰ represents, independently of each other, a group selected from:
hydrogen, C₁-alkyl, and C₁-C₂-haloalkyl,
or a stereoisomer, a tautomer, an N-oxide, a hydrate, a solvate, or a salt thereof, or a mixture of same.

6: The compound according to claim 1, which is selected from the group consisting of:
N⁵-{trans-4-[(2-chloro-4-fluorophenyl)carbamoyl]cyclohexyl}-N⁴-methyl-1H-imidazole-4,5-dicarboxamide;
N⁵-{trans-4-[(2-chloro-4-fluorophenyl)carbamoyl]cyclohexyl}-N⁴-[2-(piperidin-1-yl)ethyl]-1H-imidazole-4,5-dicarboxamide;
N⁵-{trans-4-[(2-chloro-4,5-difluorophenyl)carbamoyl]cyclohexyl}-N⁴-methyl-1H-imidazole-4,5-dicarboxamide;
N⁵-{trans-4-[(2-chloro-4,5-difluorophenyl)carbamoyl]cyclohexyl}-N⁴-[2-(piperidin-1-yl)ethyl]-1H-imidazole-4,5-dicarboxamide;
N⁵-{trans-4-[(2-chloro-4,5-dimethoxyphenyl)carbamoyl]cyclohexyl}-N⁴-methyl-1H-imidazole-4,5-dicarboxamide;
N⁵-{trans-4-[(2-chloro-4,6-difluorophenyl)carbamoyl]cyclohexyl}-N⁴-methyl-1H-imidazole-4,5-dicarboxamide;
N⁵-{trans-4-[(2-bromo-4-fluorophenyl)carbamoyl]cyclohexyl}-N⁴-methyl-1H-imidazole-4,5-dicarboxamide;
N⁵-{trans-4-[(4-fluorophenyl)carbamoyl]cyclohexyl}-N⁴-methyl-1H-imidazole-4,5-dicarboxamide;
N⁵-{trans-4-[(2-chloro-4-methylphenyl)carbamoyl]cyclohexyl}-N⁴-methyl-1H-imidazole-4,5-dicarboxamide;
N⁵-{trans-4-[(2-chloropyridin-3-yl)carbamoyl]cyclohexyl}-N⁴-methyl-1H-imidazole-4,5-dicarboxamide;
N⁵-{trans-4-[(3-chloropyridin-4-yl)carbamoyl]cyclohexyl}-N⁴-methyl-1H-imidazole-4,5-dicarboxamide;
N⁵-{trans-4-[(2-chloro-4-fluorophenyl)carbamoyl]cyclohexyl}-N⁴-[2-(4-methylpiperidin-1-yl)ethyl]-1H-imidazole-4,5-dicarboxamide;
N⁵-{trans-4-[(2-chloro-4-fluorophenyl)carbamoyl]cyclohexyl}-N⁴-[2-(morpholin-4-yl)ethyl]-1H-imidazole-4,5-dicarboxamide;
N-{trans-4-[(2-chloro-4-fluorophenyl)carbamoyl]cyclohexyl}-4-(2-oxa-6-azaspiro[3.3]hept-6-ylcarbonyl)-1H-imidazole-5-carboxamide;
N⁵-{trans-4-[(2-chloro-4-fluorophenyl)carbamoyl]cyclohexyl}-N⁴-ethyl-1H-imidazole-4,5-dicarboxamide;
N⁵-{trans-4-[(2-chloro-4-fluorophenyl)carbamoyl]cyclohexyl}-N⁴,N⁴-dimethyl-1H-imidazole-4,5-dicarboxamide;
N⁵-{trans-4-[(2-chloro-4-fluorophenyl)carbamoyl]cyclohexyl}-N⁴-cyclopropyl-1H-imidazole-4,5-dicarboxamide;
N⁵-{trans-4-[(2-chloro-4-fluorophenyl)carbamoyl]cyclohexyl}-N⁴-(2-hydroxyethyl)-1H-imidazole-4,5-dicarboxamide;
N⁵-{trans-4-[(2-chloro-4-fluorophenyl)carbamoyl]cyclohexyl}-N⁴-(2-methoxyethyl)-1H-imidazole-4,5-dicarboxamide;
N⁵-{trans-4-[(2-chloro-4-fluorophenyl)carbamoyl]cyclohexyl}-N⁴-isopropyl-1H-imidazole-4,5-dicarboxamide;
N⁵-{trans-4-[(2-chloro-4-fluorophenyl)carbamoyl]cyclohexyl}-N⁴-(2,2,2-trifluoroethyl)-1H-imidazole-4,5-dicarboxamide;
N-{trans-4-[(2-chloro-4-fluorophenyl)carbamoyl]cyclohexyl}-4-[(1,1-dioxido-1-thia-6-azaspiro[3.3]hept-6-yl)carbonyl]-1H-imidazole-5-carboxamide;
4-(azetidin-1-ylcarbonyl)-N-{trans-4-[(2-chloro-4-fluorophenyl)carbamoyl]cyclohexyl}-1H-imidazole-5-carboxamide;
4 N-{trans-4-[(2-chloro-4-fluorophenyl)carbamoyl]cyclohexyl}-4-[(3-methoxyazetidin-1-yl)carbonyl]-1H-imidazole-5-carboxamide;
N⁵-{trans-4-[(2-chloro-4-fluorophenyl)carbamoyl]cyclohexyl}-N⁴-[2-(1-oxa-6-azaspiro [3.3]hept-6-yl)ethyl]-1H-imidazole-4,5-dicarboxamide;
N-{trans-4-[(2-chloro-4-fluorophenyl)carbamoyl]cyclohexyl}-4-{[3-(dimethylamino)azetidin-1-yl]carbonyl}-1H-imidazole-5-carboxamide;
N⁵-{trans-4-[(6-bromo-2,3,4-trifluorophenyl)carbamoyl]cyclohexyl}-N⁴-methyl-1H-imidazole-4,5-dicarboxamide;
N⁵-{trans-4-[(6-bromo-2,4-difluorophenyl)carbamoyl]cyclohexyl}-N⁴-methyl-1H-imidazole-4,5-dicarboxamide;
N⁵-{trans-4-[(2-chloro-4-fluorophenyl)carbamoyl]cyclohexyl}-N⁴-methyl-1,3-oxazole-4,5-dicarboxamide;
methyl 4-({trans-4-[(2-chloro-4-fluorophenyl)carbamoyl]cyclohexyl I}carbamoyl)-1,3-oxazole-5-carboxylate;
N⁴-{(1α,2α,4β)-4-[(2-chloro-4-fluorophenyl)carbamoyl]-2-fluorocyclohexyl}-N⁵-methyl-1H-imidazole-4,5-dicarboxamide;
N⁴-{(trans)-4-[(2-chloro-4-fluorophenyl)carbamoyl]-2,2-difluorocyclohexyl}-N⁵-methyl-1H-imidazole-4,5-dicarboxamide;
N⁴-{(1R,4R)-4-[(2-chloro-4-fluorophenyl)carbamoyl]-2,2-difluorocyclohexyl}-N⁵-methyl-1H-imidazole-4,5-dicarboxamide;
N⁴-{(1 S,4S)-4-[(2-chloro-4-fluorophenyl)carbamoyl]-2,2-difluorocyclohexyl}-N⁵-methyl-1H-imidazole-4,5-dicarboxamide;
N⁴-{(1α,2β,4β)-4-[(2-chloro-4-fluorophenyl)carbamoyl]-2-methoxycyclohexyl}-N⁵-methyl-1H-imidazole-4,5-dicarboxamide;
N⁴-{(1R,2R,4R)-4-[(2-chloro-4-fluorophenyl)carbamoyl]-2-methoxycyclohexyl}-N⁵-methyl-1H-imidazole-4,5-dicarboxamide;
N⁴-{(1S,2S,4S)-4-[(2-chloro-4-fluorophenyl)carbamoyl]-2-methoxycyclohexyl}-N⁵-methyl-1H-imidazole-4,5-dicarboxamide;
N⁴-{4-[(2-chloro-4-fluorophenyl)carbamoyl]-2-methylcyclohexyl}-N⁵-methyl-1H-imidazole-4,5-dicarboxamide;
N-{4-[(2-chloro-4-fluorophenyl)carbamoyl]-2-methylcyclohexyl}-5,7-dimethylpyrazolo[1,5-a]pyrimidine-3-carboxamide;
N-{trans-4-[(2-chloro-4-fluorophenyl)carbamoyl]cyclohexyl}pyrazolo[1,5-a]pyrimidine-3-carboxamide;
N-{trans-4-[(2-chloro-4-fluorophenyl)carbamoyl]cyclohexyl}-5,7-dimethylpyrazolo[1,5-a]pyrimidine-3-carboxamide;
N-{trans-4-[(2-chloro-4-fluorophenyl)carbamoyl]cyclohexyl}imidazo[1,2-b]pyridazine-3-carboxamide;
6-acetyl-N-{trans-4-[(2-chloro-4-fluorophenyl)carbamoyl]cyclohexyl}-7-methylpyrazolo[1,5-a]pyrimidine-3-carboxamide;
6-chloro-N-{trans-4-[(2-chloro-4-fluorophenyl)carbamoyl]cyclohexyl}imidazo[1,2-b]pyridazine-3-carboxamide;
N-{trans-4-[(2-chloro-4-fluorophenyl)carbamoyl]cyclohexyl}-6-(morpholin-4-yl)imidazo[1,2-b]pyridazine-3-carboxamide;
N-{trans-4-[(2-chloro-4-fluorophenyl)carbamoyl]cyclohexyl}-6-(4-methylpiperazin-1-yl)imidazo[1,2-b]pyridazine-3-carboxamide;
N-{trans-4-[(2-chloro-4-fluorophenyl)carbamoyl]cyclohexyl}-6-methoxyimidazo[1,2-b]pyridazine-3-carboxamide;
N-{trans-4-[(2-chloro-4-fluorophenyl)carbamoyl]cyclohexyl}-6-(pyrrolidin-1-yl)imidazo[1,2-b]pyridazine-3-carboxamide;
N-{trans-4-[(2-chloro-4-fluorophenyl)carbamoyl]cyclohexyl}-6-(dimethylamino)imidazo[1,2-b]pyridazine-3-carboxamide;
N-{trans-4-[(2-chloro-4-fluorophenyl)carbamoyl]cyclohexyl}-6-(methylamino)imidazo[1,2-b]pyridazine-3-carboxamide;
N-{trans-4-[(2-chloro-4-fluorophenyl)carbamoyl]cyclohexyl}-7-methyl-6-[1-(methylamino)ethyl]pyrazolo[1,5-a]pyrimidine-3-carboxamide formic acid salt;
N-{trans-4-[(2-chloro-4-fluorophenyl)carbamoyl]cyclohexyl}-7-methyl-6-[1-(methylamino)ethyl]pyrazolo[1,5-a]pyrimidine-3-carboxamide;
N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide formic acid salt;
N-{trans-4-[(2-chloro-4-fluorophenyl)carbamoyl]cyclohexyl}-6-[1-(dimethylamino)ethyl]-7-methylpyrazolo[1,5-a]pyrimidine-3-carboxamide;
N-{trans-4-[(2-chloro-4-fluorophenyl)carbamoyl]cyclohexyl}-6-[(1R)-(dimethylamino)ethyl]-7-methylpyrazolo[1,5-a]pyrimidine-3-carboxamide;
N-{trans-4-[(2-chloro-4-fluorophenyl)carbamoyl]cyclohexyl}-6-(1-hydroxyethyl)-7-methylpyrazolo[1,5-a]pyrimidine-3-carboxamide;
2-amino-N-{trans-4-[(2-chloro-4-fluorophenyl)carbamoyl]cyclohexyl}pyrazolo[1,5-a]pyrimidine-3-carboxamide;
7-tert-butyl-N-{trans-4-[(2-chloro-4-fluorophenyl)carbamoyl]cyclohexyl}-5-methylpyrazolo[1,5-a]pyrimidine-3-carboxamide;
N-{trans-4-[(2-chloro-4-fluorophenyl)carbamoyl]cyclohexyl}-6-methylpyrazolo[1,5-a]pyrimidine-3-carboxamide;
N-{trans-4-[(2-chloro-4-fluorophenyl)carbamoyl]cyclohexyl}-5-cyclopropyl-7-methylpyrazolo[1,5-a]pyrimidine-3-carboxamide;
N⁵-{trans-4-[(2-chloro-4-fluoro-5-methylphenyl)carbamoyl]cyclohexyl}-N⁴-methyl-1H-imidazole-4,5-dicarboxamide;
N⁵-{trans-4-[(2-chloro-4-fluoro-5-methylphenyl)carbamoyl]cyclohexyl}-N⁴-(tetrahydrofuran-2-ylmethyl)-1H-imidazole-4,5-dicarboxamide;
N⁵-{trans-4-[(2-chloro-4,6-difluorophenyl)carbamoyl]cyclohexyl}-N⁴-(1-methoxypropan-2-yl)-1H-imidazole-4,5-dicarboxamide;
N⁵-{trans-4-[(2-chloro-4,6-difluorophenyl)carbamoyl]cyclohexyl}-N⁴-[(2S)-1-methoxypropan-2-yl]-1H-imidazole-4,5-dicarboxamide;
N⁵-{trans-4-[(2-chloro-4,6-difluorophenyl)carbamoyl]cyclohexyl}-N⁴-[(2R)-1-methoxypropan-2-yl]-1H-imidazole-4,5-dicarboxamide;
N⁵-{trans-4-[(2-chloro-4,6-difluorophenyl)carbamoyl]cyclohexyl}-N⁴-(2-methoxypropyl)-1H-imidazole-4,5-dicarboxamide;
N⁵-{trans-4-[(2-chloro-4,6-difluorophenyl)carbamoyl]cyclohexyl}-N⁴-(tetrahydrofuran-2-ylmethyl)-1H-imidazole-4,5-dicarboxamide;
N⁵-{trans-4-[(2-chloro-4,6-difluorophenyl)carbamoyl]cyclohexyl}-N⁴-[(2R)-tetrahydrofuran-2-ylmethyl]-1H-imidazole-4,5-dicarboxamide;
N⁵-{trans-4-[(2-chloro-4,6-difluorophenyl)carbamoyl]cyclohexyl}-N⁴-[(2R)-tetrahydrofuran-2-ylmethyl]-1H-imidazole-4,5-dicarboxamide;
N-{trans-4-[(2-chloro-4-fluorophenyl)carbamoyl]cyclohexyl}-4-[(3,3-difluoroazetidin-1-yl)carbonyl]-1H-imidazole-5-carboxamide;
N⁵-{trans-4-[(2-chloro-4-fluorophenyl)carbamoyl]cyclohexyl}-N⁴-(2-methoxy-2-methylpropyl)-1H-imidazole-4,5-dicarboxamide;
N⁵-{trans-4-[(2-chloro-4-fluorophenyl)carbamoyl]cyclohexyl}-N⁴-(2-cyclopropylethyl)-1H-imidazole-4,5-dicarboxamide;
N⁵-{trans-4-[(2-chloro-4-fluorophenyl)carbamoyl]cyclohexyl}-N⁴-(2-hydroxy-2-methylpropyl)-1H-imidazole-4,5-dicarboxamide;
N⁵-{trans-4-[(2-chloro-4-fluorophenyl)carbamoyl]cyclohexyl}-N⁴-(2-isopropoxyethyl)-1H-imidazole-4,5-dicarboxamide;
N⁵-{trans-4-[(2-chloro-4-fluorophenyl)carbamoyl]cyclohexyl}-N⁴-[2-(isopropylamino)ethyl]-1H-imidazole-4,5-dicarboxamide;
N⁵-{trans-4-[(2-chloro-4-fluorophenyl)carbamoyl]cyclohexyl}-N⁴-(tetrahydrofuran-2-ylmethyl)-1H-imidazole-4,5-dicarboxamide;
N⁵-{trans-4-[(2-chloro-4-fluorophenyl)carbamoyl]cyclohexyl}-N⁴-[(2S)-tetrahydrofuran-2-ylmethyl]-l 1H-imidazole-4,5-dicarboxamide;
N⁵-{trans-4-[(2-chloro-4-fluorophenyl)carbamoyl]cyclohexyl}-N⁴-[(2R)-tetrahydrofuran-2-ylmethyl]-l 1H-imidazole-4,5-dicarboxamide;
N⁵-{trans-4-[(2-chloro-4-fluorophenyl)carbamoyl]cyclohexyl}-N⁴-(2-methoxypropyl)-1H-imidazole-4,5-dicarboxamide;
N⁵-{trans-4-[(2-chloro-4-fluorophenyl)carbamoyl]cyclohexyl}-N⁴-(1-methoxypropan-2-yl)-1H-imidazole-4,5-dicarboxamide;
N⁵-{trans-4-[(2-chloro-4-fluorophenyl)carbamoyl]cyclohexyl}-N⁴-[(2S)-1-methoxypropan-2-yl]-1H-imidazole-4,5-dicarboxamide;
N⁵-{trans-4-[(2-chloro-4-fluorophenyl)carbamoyl]cyclohexyl}-N⁴-[(2R)-1-methoxypropan-2-yl]-1H-imidazole-4,5-dicarboxamide;
N⁵-{trans-4-[(2-chloro-4,5-difluorophenyl)carbamoyl]cyclohexyl}-N⁴-(2-methoxy-2-methylpropyl)-1H-imidazole-4,5-dicarboxamide;
N⁵-{trans-4-[(2-chloro-4,5-difluorophenyl)carbamoyl]cyclohexyl}-N⁴-(tetrahydrofuran-2-ylmethyl)-1H-imidazole-4,5-dicarboxamide;
N⁵-{trans-4-[(2-chloro-4,5-difluorophenyl)carbamoyl]cyclohexyl}-N⁴-(1-methoxypropan-2-yl)-1H-imidazole-4,5-dicarboxamide;
N⁵-(trans-4-{[2-chloro-5-(morpholin-4-yl)phenyl]carbamoyl}cyclohexyl)-N⁴-methyl-1H-imidazole-4,5-dicarboxamide;
N⁵-(trans-4-{[2-chloro-5-(morpholin-4-yl)phenyl]carbamoyl}cyclohexyl)-N⁴-(tetrahydrofuran-2-ylmethyl)-1H-imidazole-4,5-dicarboxamide;
N⁵-(trans-4-{[2-chloro-5-(morpholin-4-yl)phenyl]carbamoyl}cyclohexyl)-N⁴-(2-methoxy-2-methylpropyl)-1H-imidazole-4,5-dicarboxamide;
N⁵-(trans-4-{[2-chloro-5-(morpholin-4-yl)phenyl]carbamoyl}cyclohexyl)-N⁴-(1-methoxypropan-2-yl)-1H-imidazole-4,5-dicarboxamide;
N-(trans-4-{[2-chloro-5-(morpholin-4-yl)phenyl]carbamoyl}cyclohexyl)-4-[(3,3-difluoroazetidin-1-yl)carbonyl]-1H-imidazole-5-carboxamide;
N⁵-(trans-4-{[2-chloro-5-(morpholin-4-yl)phenyl]carbamoyl}cyclohexyl)-N⁴-(2-cyclopropylethyl)-1H-imidazole-4,5-dicarboxamide;
N⁵-(trans-4-{[2-chloro-5-(morpholin-4-yl)phenyl]carbamoyl}cyclohexyl)-N⁴-(2-hydroxy-2-methylpropyl)-1H-imidazole-4,5-dicarboxamide;
N⁵-(trans-4-{[2-chloro-5-(morpholin-4-yl)phenyl]carbamoyl}cyclohexyl)-N⁴-(2,2,2-trifluoroethyl)-1H-imidazole-4,5-dicarboxamide;
N⁵-{trans-4-[(4-chloropyridin-3-yl)carbamoyl]cyclohexyl}-N⁴-methyl-1H-imidazole-4,5-dicarboxamide;
N⁵-{trans-4-[(4-chloropyridin-3-yl)carbamoyl]cyclohexyl}-N⁴-(tetrahydrofuran-2-ylmethyl)-1H-imidazole-4,5-dicarboxamide;
N⁵-{trans-4-[(4-chloropyridin-3-yl)carbamoyl]cyclohexyl}-N⁴-(2-methoxy-2-methylpropyl)-1H-imidazole-4,5-dicarboxamide;
N⁵-{trans-4-[(4-chloropyridin-3-yl)carbamoyl]cyclohexyl}-N⁴-(1-methoxypropan-2-yl)-1H-imidazole-4,5-dicarboxamide;
N⁵-{trans-4-[(2-chloro-4-fluoro-5-methylphenyl)carbamoyl]cyclohexyl}-N⁴-(2-hydroxy-2-methylpropyl)-1H-imidazole-4,5-dicarboxamide;
N⁵-{trans-4-[(2-chloro-4-fluoro-5-methylphenyl)carbamoyl]cyclohexyl}-N⁴-(2-methoxy-2-methylpropyl)-1H-imidazole-4,5-dicarboxamide;
N-{trans-4-[(3-chloropyridin-4-yl)carbamoyl]cyclohexyl}-5,7-dimethylpyrazolo[1,5-a]pyrimidine-3-carboxamide;
N-{trans-4-[(3-chloropyridin-4-yl)carbamoyl]cyclohexyl}imidazo[1,2-b]pyridazine-3-carboxamide;
N-{trans-4-[(3-chloropyridin-4-yl)carbamoyl]cyclohexyl}-6-methylpyrazolo[1,5-a]pyrimidine-3-carboxamide;
N-{trans-4-[(3-chloropyridin-4-yl)carbamoyl]cyclohexyl}pyrazolo[1,5-a]pyrimidine-3-carboxamide;
ethyl 3-({trans-4-[(2-chloro-4-fluorophenyl)carbamoyl]cyclohexyl}carbamoyl)pyrazolo[1,5-a]pyrimidine-6-carboxylate;
N-{trans-4-[(2-chloro-4-fluorophenyl)carbamoyl]cyclohexyl}-5-(morpholin-4-yl)pyrazolo[1,5-a]pyrimidine-3-carboxamide;
N-{trans-4-[(2-chloro-4-fluorophenyl)carbamoyl]cyclohexyl}-6-(pyrrolidin-1-yl)pyrazolo[1,5-a]pyrimidine-3-carboxamide;
N-{trans-4-[(2-chloro-4-fluorophenyl)carbamoyl]cyclohexyl}-6-(4-methylpiperazin-1-yl)pyrazolo[1,5-a]pyrimidine-3-carboxamide;
N-{trans-4-[(2-chloro-4-fluorophenyl)carbamoyl]cyclohexyl}-5-(methoxymethyl)-7-methylpyrazolo[1,5-a]pyrimidine-3-carboxamide;
N-{trans-4-[(2-chloro-4-fluorophenyl)carbamoyl]cyclohexyl}-7-(methoxymethyl)-5-methylpyrazolo[1,5-a]pyrimidine-3-carboxamide;
N-{trans-4-[(2-chloro-4,6-difluorophenyl)carbamoyl]cyclohexyl}-5-(methoxymethyl)-7-methylpyrazolo[1,5-a]pyrimidine-3-carboxamide;
N-{trans-4-[(2-chloro-4-fluorophenyl)carbamoyl]cyclohexyl}-5-methyl-7,8-dihydro-6H-cyclopenta[e]pyrazolo[1,5-a]pyrimidine-3-carboxamide;
N-{trans-4-[(2-chloro-4-fluorophenyl)carbamoyl]cyclohexyl}-6-[(2-hydroxypropyl)amino]imidazo[1,2-b]pyridazine-3-carboxamide;
N⁴-{4-[(2-chloro-4-fluorophenyl)carbamoyl]bicyclo[2.2.2]oct-1-yl}-N⁵-methyl-1H-imidazole-4,5-dicarboxamide;
N⁴-{4-[(2-chloro-4,6-difluorophenyl)carbamoyl]bicyclo[2.2.2]oct-1-yl}-N⁵-methyl-H-imidazole-4,5-dicarboxamide;
N-{4-[(2-chloro-4-fluorophenyl)carbamoyl]bicyclo[2.2.2]oct-1-yl}pyrazolo[1,5-a]pyrimidine-3-carboxamide;
N-{4-[(2-chloro-4-fluoro-5-methylphenyl)carbamoyl]bicyclo[2.2.2]oct-1-yl}pyrazolo[1,5-a]pyrimidine-3-carboxamide;
N-{4-[(2-chloro-4,6-difluorophenyl)carbamoyl]bicyclo[2.2.2]oct-1-yl}pyrazolo[1,5-a]pyrimidine-3-carboxamide;
N-{4-[(4-chloropyridin-3-yl)carbamoyl]bicyclo[2.2.2]oct-1-yl}pyrazolo[1,5-a]pyrimidine-3-carboxamide;
N-{4-[(2-chloro-4-fluorophenyl)carbamoyl]bicyclo[2.2.2]oct-1-yl}imidazo[1,2-b]pyridazine-3-carboxamide;
N-{4-[(2-chloro-4-fluoro-5-methylphenyl)carbamoyl]bicyclo[2.2.2]oct-1-yl}imidazo[1,2-b]pyridazine-3-carboxamide;
N-{4-[(2-chloro-4,6-difluorophenyl)carbamoyl]bicyclo[2.2.2]oct-1-yl}imidazo[1,2-b]pyridazine-3-carboxamide;
N-{4-[(4-chloropyridin-3-yl)carbamoyl]bicyclo[2.2.2]oct-1-yl}imidazo[1,2-b]pyridazine-3-carboxamide;
N-{4-[(2-chloro-4-fluorophenyl)carbamoyl]bicyclo[2.2.2]oct-1-yl}-5,7-dimethylpyrazolo[1,5-a]pyrimidine-3-carboxamide;
N-{4-[(2-chloro-4-fluoro-5-methylphenyl)carbamoyl]bicyclo[2.2.2]oct-1-yl}-5,7-dimethylpyrazolo[1,5-a]pyrimidine-3-carboxamide;
N-{4-[(2-chloro-4,6-difluorophenyl)carbamoyl]bicyclo[2.2.2]oct-1-yl}-5,7-dimethylpyrazolo[1,5-a]pyrimidine-3-carboxamide;
N-{4-[(4-chloropyridin-3-yl)carbamoyl]bicyclo[2.2.2]oct-1-yl}-5,7-dimethylpyrazolo[1,5-a]pyrimidine-3-carboxamide;
N⁵-{trans-4-[(2-chloro-5-fluorophenyl)carbamoyl]cyclohexyl}-N⁴-(tetrahydrofuran-2-ylmethyl)-1H-imidazole-4,5-dicarboxamide;
N-{trans-4-[(2-chloro-4,5-difluorophenyl)carbamoyl]cyclohexyl}pyrazolo[1,5-a]pyrimidine-3-carboxamide;
N-{trans-4-[(2-chloro-4,6-difluorophenyl)carbamoyl]cyclohexyl}pyrazolo[1,5-a]pyrimidine-3-carboxamide;
N-{trans-4-[(2-chloro-4,5-difluorophenyl)carbamoyl]cyclohexyl}imidazo[1,2-b]pyridazine-3-carboxamide;
N-{trans-4-[(2-chloro-4,6-difluorophenyl)carbamoyl]cyclohexyl}imidazo[1,2-b]pyridazine-3-carboxamide;
N-{trans-4-[(2-chloro-4,5-difluorophenyl)carbamoyl]cyclohexyl}-5,7-dimethylpyrazolo[1,5-a]pyrimidine-3-carboxamide;
N-{trans-4-[(2-chloro-4,6-difluorophenyl)carbamoyl]cyclohexyl}-5,7-dimethylpyrazolo[1,5-a]pyrimidine-3-carboxamide;
N-[(trans)-2-(2-chloro-4-fluorophenyl)-1-oxo-2-azaspiro[4.5]dec-8-yl]pyrazolo[1,5-a]pyrimidine-3-carboxamide;
N-[(trans)-2-(2-chloro-4-fluorophenyl)-1-oxo-2-azaspiro[4.5]dec-8-yl]-6-methylpyrazolo[1,5-a]pyrimidine-3-carboxamide;
N-[(trans)-2-(2-chloro-4-fluorophenyl)-1-oxo-2-azaspiro[4.5]dec-8-yl]imidazo[1,2-b]pyridazine-3-carboxamide;
N-[(trans)-2-(2-chloro-4-fluorophenyl)-1-oxo-2-azaspiro[4.5]dec-8-yl]-5,7-dimethylpyrazolo[1,5-a]pyrimidine-3-carboxamide;
N⁴-[(trans)-2-(2-chloro-4-fluorophenyl)-1-oxo-2-azaspiro [4.5]dec-8-yl]-N⁵-methyl-1H-imidazole-4,5-dicarboxamide;
N⁴-[(trans)-2-(2-chloro-4-fluorophenyl)-1-oxo-2-azaspiro [4.5]dec-8-yl]-N⁵-ethyl-1H-imidazole-4,5-dicarboxamide;
N⁵-(trans-4-{[2-chloro-5-(trifluoromethyl)phenyl]carbamoyl}cyclohexyl)-N⁴-methyl-1H-imidazole-4,5-dicarboxamide;
N⁵-(trans-4-{[2-chloro-5-(trifluoromethyl)phenyl]carbamoyl}cyclohexyl)-N⁴-(1-methoxypropan-2-yl)-1H-imidazole-4,5-dicarboxamide;
N⁵-(trans-4-{[2-chloro-5-(trifluoromethyl)phenyl]carbamoyl}cyclohexyl)-N⁴-(2-methoxy-2-methylpropyl)-1H-imidazole-4,5-dicarboxamide;
N⁵-(trans-4-{[2-chloro-5-(trifluoromethyl)phenyl]carbamoyl}cyclohexyl)-N⁴-(2-hydroxy-2-methylpropyl)-1H-imidazole-4,5-dicarboxamide;
N⁵-(trans-4-{[2-chloro-5-(trifluoromethyl)phenyl]carbamoyl}cyclohexyl)-N⁴-(tetrahydrofuran-2-ylmethyl)-1H-imidazole-4,5-dicarboxamide;
N⁵-{trans-4-[(5-chloropyrimidin-4-yl)carbamoyl]cyclohexyl}-N⁴-methyl-1H-imidazole-4,5-dicarboxamide;
N-[(cis)-2-(2-chloro-4-fluorophenyl)-1-oxo-2-azaspiro[4.5]dec-8-yl]pyrazolo[1,5-a]pyrimidine-3-carboxamide;
N-(trans-4-{[4-chloro-6-(trifluoromethyl)pyridin-3-yl]carbamoyl}cyclohexyl)pyrazolo[1,5-a]pyrimidine-3-carboxamide;
N-{trans-4-[(2-chloro-4-fluoro-5-methylphenyl)carbamoyl]cyclohexyl}pyrazolo[1,5-a]pyrimidine-3-carboxamide;
N-{trans-4-[(2-chloro-4-fluoro-6-methylphenyl)carbamoyl]cyclohexyl}pyrazolo[1,5-a]pyrimidine-3-carboxamide;
N-{trans-4-[(2-chloro-6-cyano-4-fluorophenyl)carbamoyl]cyclohexyl}pyrazolo[1,5-a]pyrimidine-3-carboxamide;
N-{trans-4-[(5-chloro-2-methylpyridin-4-yl)carbamoyl]cyclohexyl}pyrazolo[1,5-a]pyrimidine-3-carboxamide;
N-{trans-4-[(2-chloro-4-fluorophenyl)(ethyl)carbamoyl]cyclohexyl}pyrazolo[1,5-a]pyrimidine-3-carboxamide;
N-{trans-4-[(2-chloro-4-fluorophenyl)(methyl)carbamoyl]cyclohexyl}pyrazolo[1,5-a]pyrimidine-3-carboxamide;
N-{trans-4-[methyl(phenyl)carbamoyl]cyclohexyl}pyrazolo[1,5-a]pyrimidine-3-carboxamide;
N⁵-{trans-4-[(2-chloro-4-fluorophenyl)(2-hydroxyethyl)carbamoyl]cyclohexyl}-N⁴-methyl-1H-imidazole-4,5-dicarboxamide;
N⁵-{trans-4-[(2-chlorophenyl)(ethyl)carbamoyl]cyclohexyl}-N⁴-methyl-1H-imidazole-4,5-dicarboxamide;
N⁵-[(trans)-2-(2-chloro-4-fluorophenyl)-1-oxo-2-azaspiro[5.5]undec-9-yl]-N⁴-methyl-1H-imidazole-4,5-dicarboxamide);
N⁵-[(trans)-2-(2-chloro-4-fluorophenyl)-1-oxo-2-azaspiro[4.5]dec-8-yl]-N⁴-[2-(4-methylpiperidin-1-yl)ethyl]-1H-imidazole-4,5-dicarboxamide;
N⁵-[(trans)-2-(2-chloro-4-fluorophenyl)-1-oxo-2-azaspiro[4.5]dec-8-yl]-N⁴-[2-(piperidin-1-yl)ethyl]-1H-imidazole-4,5-dicarboxamide;
N⁵-[(trans)-2-(2-chloro-4-fluorophenyl)-1-oxo-2-azaspiro [4.5]dec-8-yl]-N⁴-isopropyl-1H-imidazole-4,5-dicarboxamide;
N⁵-[(trans)-2-(2-chloro-4-fluorophenyl)-1-oxo-2-azaspiro[4.5]dec-8-yl]-N⁴-(2-methoxyethyl)-1H-imidazole-4,5-dicarboxamide;
N⁵-[(trans)-2-(2-chloro-4-fluorophenyl)-1-oxo-2-azaspiro[4.5]dec-8-yl]-N⁴-(1-methoxypropan-2-yl)-1H-imidazole-4,5-dicarboxamide;
N⁵-[(trans)-2-(2-chloro-4-fluorophenyl)-1-oxo-2-azaspiro [4.5]dec-8-yl]-N⁴-cyclopropyl-1H-imidazole-4,5-dicarboxamide;
N-[(trans)-2-(2-chloro-4-fluorophenyl)-1-oxo-2-azaspiro[4.5]dec-8-yl]-4-[(3-fluoroazetidin-1-yl)carbonyl]-1H-imidazole-5-carboxamide;
N-[(trans)-2-(2-chloro-4-fluorophenyl)-1-oxo-2-azaspiro[4.5]dec-8-yl]-4-{[4-(2,2,2-trifluoroethyl)piperazin-1-yl]carbonyl}-1H-imidazole-5-carboxamide;
N-[2-(2-chloro-4,5-difluorophenyl)-1-oxo-2-azaspiro[4.5]dec-8-yl]pyrazolo[1,5-a]pyrimidine-3-carboxamide (isomer 1);
N-{trans-4-[(4-chloropyridin-3-yl)carbamoyl]cyclohexyl}-5,7-dimethylpyrazol[1,5-a]pyrimidine-3-carboxamide;
N⁵-(trans-4-{[2-chloro-5-(2-hydroxypropan-2-yl)phenyl]carbamoyl}cyclohexyl)-N⁴-methyl-1H-imidazole-4,5-dicarboxamide;
N⁴-methyl-N⁵-{trans-4-[methyl(phenyl)carbamoyl]cyclohexyl}-1H-imidazole-4,5-dicarboxamide;
N⁵-{trans-4-[(4-fluorophenyl)(methyl)carbamoyl]cyclohexyl}-N⁴-methyl-1H-imidazole-4,5-dicarboxamide;
N⁵-{trans-4-[(2-chloro-4-fluorophenyl)(methyl)carbamoyl]cyclohexyl}-N⁴-methyl-1H-imidazole-4,5-dicarboxamide;
N⁵-{trans-4-[(2-chloro-6-cyano-4-fluorophenyl)carbamoyl]cyclohexyl}-N⁴-methyl-1H-imidazole-4,5-dicarboxamide;
N⁵-{trans-4-[(4-chloro-3-methyl-1,2-thiazol-5-yl)carbamoyl]cyclohexyl}-N⁴-methyl-1H-imidazole-4,5-dicarboxamide;
N-{trans-4-[(2-chloro-4-fluoro-5-methylphenyl)(methyl)carbamoyl]cyclohexyl}pyrazolo[1,5-a]pyrimidine-3-carboxamide;
N-{trans-4-[(2-chloro-4-fluorophenyl)(cyclopropylmethyl)carbamoyl]cyclohexyl}pyrazolo[1,5-a]pyrimidine-3-carboxamide;
N-{trans-4-[methyl(2-methylphenyl)carbamoyl]cyclohexyl}pyrazolo[1,5-a]pyrimidine-3-carboxamide;
N-{trans-4-[(2-chloro-4-fluorophenyl)carbamoyl]-4-methylcyclohexyl}pyrazolo[1,5-a]pyrimidine-3-carboxamide;
N-{cis-4-[(2-chloro-4-fluorophenyl)carbamoyl]-4-methylcyclohexyl}pyrazolo[1,5-a]pyrimidine-3-carboxamide;
N⁴-{cis-4-[(2-chloro-4-fluorophenyl)carbamoyl]-4-methylcyclohexyl}-N⁵-methyl-1H-imidazole-4,5-dicarboxamide;
N⁵-[(cis)-2-(2-chloro-4-fluorophenyl)-1-oxo-2-azaspiro[4.5]dec-8-yl]-N⁴-methyl-1H-imidazole-4,5-dicarboxamide;
N-[(trans)-2-(2-chloro-4-fluorophenyl)-1-oxo-2-azaspiro[4.5]dec-8-yl]-4-[(6-methyl-1,1-dioxido-1,4-thiazepan-4-yl)carbonyl]-1H-imidazole-5-carboxamide;
N-[(trans)-2-(2-chloro-4-fluorophenyl)-1-oxo-2-azaspiro[4.5]dec-8-yl]-4-[(2-methyl-2,6-diazaspiro[3.4]oct-6-yl)carbonyl]-1H-imidazole-5-carboxamide;
N-[(trans)-2-(2-chloro-4-fluorophenyl)-1-oxo-2-azaspiro[4.5]dec-8-yl]-4-[(6-methyl-2,6-diazaspiro[3.3]hept-2-yl)carbonyl]-1H-imidazole-5-carboxamide;
N⁵-[(trans)-2-(2-chloro-4-fluorophenyl)-1-oxo-2-azaspiro[4.5]dec-8-yl]-1H-imidazole-4,5-dicarboxamide;
N⁵-[(trans)-2-(2-chloro-4-fluorophenyl)-1-oxo-2-azaspiro[4.5]dec-8-yl]-N⁴-(1-methylazetidin-3-yl)-1H-imidazole-4,5-dicarboxamide;
N-[(trans)-2-(2-chloro-4-fluorophenyl)-1-oxo-2-azaspiro[4.5]dec-8-yl]-4-[(3-methoxyazetidin-1-yl)carbonyl]-1H-imidazole-5-carboxamide;
N-[(trans)-2-(2-chloro-4-fluorophenyl)-1-oxo-2-azaspiro[4.5]dec-8-yl]-4-[(3,3-difluoroazetidin-1-yl)carbonyl]-1H-imidazole-5-carboxamide;
N-[(trans)-2-(2-chloro-4-fluorophenyl)-1-oxo-2-azaspiro[4.5]dec-8-yl]-4-{[3-(difluoromethyl)azetidin-1-yl]carbonyl}-1H-imidazole-5-carboxamide;
N-[(trans)-2-(2-chloro-4-fluorophenyl)-1-oxo-2-azaspiro[4.5]dec-8-yl]-4-[(3-hydroxyazetidin-1-yl)carbonyl]-1H-imidazole-5-carboxamide;
N-[(trans)-2-(2-chloro-4-fluorophenyl)-1-oxo-2-azaspiro[4.5]dec-8-yl]-4-{[3-(difluoromethoxy)azetidin-1-yl]carbonyl}-1H-imidazole-5-carboxamide;
N-[(trans)-2-(2-chloro-4-fluorophenyl)-1-oxo-2-azaspiro[4.5]dec-8-yl]-4-(2-oxa-6-azaspiro [3.3]hept-6-ylcarbonyl)-1H-imidazole-5-carboxamide;
N⁴-[(trans)-2-(2-chloro-4-fluorophenyl)-1-oxo-2-azaspiro [4.5]dec-8-yl]-N⁵-(2,2,2-trifluoroethyl)-1H-imidazole-4,5-dicarboxamide;
N⁵-[(trans)-2-(2-chloro-4,5-difluorophenyl)-1-oxo-2-azaspiro [4.5]dec-8-yl]-N⁴-methyl-1H-imidazole-4,5-dicarboxamide;
N⁵-[(trans)-2-(2-chloro-4,5-difluorophenyl)-1-oxo-2-azaspiro [4.5]dec-8-yl]-N⁴-ethyl-1H-imidazole-4,5-dicarboxamide;
N⁵-[(trans)-2-(2-chloro-4,5-difluorophenyl)-1-oxo-2-azaspiro [4.5]dec-8-yl]-N⁴-(propan-2-yl)-1H-imidazole-4,5-dicarboxamide;
N⁵-[(trans)-2-(2-chloro-4,5-difluorophenyl)-1-oxo-2-azaspiro [4.5]dec-8-yl]-N⁴-cyclopropyl-1H-imidazole-4,5-dicarboxamide;
N-[(trans)-2-(2-chloro-4,5-difluorophenyl)-1-oxo-2-azaspiro[4.5]dec-8-yl]-4-[(3-fluoroazetidin-1-yl)carbonyl]-1H-imidazole-5-carboxamide;
N⁵-[(trans)-2-(2-chloro-4,5-difluorophenyl)-1-oxo-2-azaspiro[4.5]dec-8-yl]-N⁴-(2,2,2-trifluoroethyl)-1H-imidazole-4,5-dicarboxamide;
N⁵-[(trans)-2-(2-chloro-4,5-difluorophenyl)-1-oxo-2-azaspiro[4.5]dec-8-yl]-N⁴-(2-methoxyethyl)-1H-imidazole-4,5-dicarboxamide;
N⁴-methyl-N⁵-{trans-4-[methyl(2-methylphenyl)carbamoyl]cyclohexyl}-1H-imidazole-4,5-dicarboxamide;
N-{trans-4-[(2-chloro-4-fluorophenyl)carbamoyl]cyclohexyl}-7-cyclopropyl-5-methylpyrazolo[1,5-a]pyrimidine-3-carboxamide;
N-{trans-4-[(2-chloro-4-fluorophenyl)carbamoyl]cyclohexyl}pyrazolo[1,5-a]pyridine-3-carboxamide;
N-{trans-4-[(2-chloro-4-fluorophenyl)carbamoyl]cyclohexyl}imidazo[1,2-a]pyridine-3-carboxamide;
N-[(cis)-2-(2-chloro-4-fluorophenyl)-1-oxo-2-azaspiro[4.5]dec-8-yl]-5,7-dimethylpyrazolo[1,5-a]pyrimidine-3-carboxamide;
N-[(cis)-2-(4-fluorophenyl)-1-oxo-2-azaspiro[4.5]dec-8-yl]-5,7-dimethylpyrazolo[1,5-a]pyrimidine-3-carboxamide;
N-[(trans)-2-(4-fluorophenyl)-1-oxo-2-azaspiro [4.5]dec-8-yl]pyrazolo[1,5-a]pyrimidine-3-carboxamide;
N-[(trans)-2-(4-fluorophenyl)-1-oxo-2-azaspiro[4.5]dec-8-yl]-6-methylpyrazolo[1,5-a]pyrimidine-3-carboxamide;
N-[(trans)-2-(4-fluorophenyl)-1-oxo-2-azaspiro[4.5]dec-8-yl]-5,7-dimethylpyrazolo[1,5-a]pyrimidine-3-carboxamide;
N⁴-[(trans)-2-(4-fluorophenyl)-1-oxo-2-azaspiro[4.5]dec-8-yl]-N⁵-methyl-1H-imidazole-4,5-dicarboxamide;
N⁵-ethyl-N⁴-[(trans)-2-(4-fluorophenyl)-1-oxo-2-azaspiro [4.5]dec-8-yl]-1H-imidazole-4,5-dicarboxamide;
N-[(trans)-2-(2-chloro-4-fluorophenyl)-1-oxo-2-azaspiro[4.5]dec-8-yl]-6-(2-hydroxyethyl)pyrazolo[1,5-a]pyrimidine-3-carboxamide;
N-[(trans)-2-(2-chloro-4-fluorophenyl)-1-oxo-2-azaspiro[4.5]dec-8-yl]-5-(methylamino)pyrazolo[1,5-a]pyrimidine-3-carboxamide;
N-[(trans)-2-(2-chloro-4-fluorophenyl)-1-oxo-2-azaspiro[4.5]dec-8-yl]-2-methylpyrazolo[1,5-a]pyrimidine-3-carboxamide;
N-[(trans)-2-(2-chloro-4-fluorophenyl)-1-oxo-2-azaspiro[4.5]dec-8-yl]-5-(dimethylamino)pyrazolo[1,5-a]pyrimidine-3-carboxamide;
N-[(trans)-2-(2-chloro-4-fluorophenyl)-1-oxo-2-azaspiro[4.5]dec-8-yl]-5-methoxypyrazolo[1,5-a]pyrimidine-3-carboxamide;
N-[(trans)-2-(2-chloro-4-fluorophenyl)-1-oxo-2-azaspiro [4.5]dec-8-yl]-7-(methoxymethyl)-5-methylpyrazolo[1,5-a]pyrimidine-3-carboxamide;
N-[(trans)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide;
N-[(trans)-2-(2-chloro-4-fluorophenyl)-1-oxo-2-azaspiro[4.5]dec-8-yl]-5-methylpyrazolo[1,5-a]pyrimidine-3-carboxamide;
N-[(trans)-2-(2-chloro-4-fluorophenyl)-1-oxo-2-azaspiro[4.5]dec-8-yl]-7-methylpyrazolo[1,5-a]pyrimidine-3-carboxamide;
N-[(trans)-2-(2-chloro-4-fluorophenyl)-1-oxo-2-azaspiro[4.5]dec-8-yl]imidazo[1,2-a]pyridine-3-carboxamide;
N-[(trans)-2-(2-chloro-4-fluorophenyl)-1-oxo-2-azaspiro [4.5]dec-8-yl]-7-(difluoromethyl)-5-methylpyrazolo[1,5-a]pyrimidine-3-carboxamide;
tert-butyl 4-(3-{[(trans)-2-(2-chloro-4-fluorophenyl)-1-oxo-2-azaspiro [4.5]dec-8-yl]carbamoyl}pyrazolo[1,5-a]pyrimidin-5-yl)piperazine-1-carboxylate;
N-[(trans)-2-(2-chloro-4-fluorophenyl)-1-oxo-2-azaspiro[4.5]dec-8-yl]-5-(piperazin-1-yl)pyrazolo[1,5-a]pyrimidine-3-carboxamide hydrochloric acid salt;
N-[(trans)-2-(2-chloro-4-fluorophenyl)-1-oxo-2-azaspiro[4.5]dec-8-yl]-2,5,7-trimethylpyrazolo[1,5-a]pyrimidine-3-carboxamide;
N-[(trans)-2-(2-chloro-4-fluorophenyl)-1-oxo-2-azaspiro[4.5]dec-8-yl]-1H-pyrazolo[4,3-b]pyridine-3-carboxamide;
N-[(trans)-2-(2-chloro-4-fluorophenyl)-1-oxo-2-azaspiro[4.5]dec-8-yl]-5-(4-methylpiperazin-1-yl)pyrazolo[1,5-a]pyrimidine-3-carboxamide;
N-[(trans)-2-(2-chloro-4-fluorophenyl)-1-oxo-2-azaspiro[4.5]dec-8-yl]pyrazolo[1,5-a]pyridine-3-carboxamide;
N-[(trans)-2-(2-chloro-4-fluorophenyl)-1-oxo-2-azaspiro[4.5]dec-8-yl]-5-(morpholin-4-yl)pyrazolo[1,5-a]pyrimidine-3-carboxamide;
N-[(trans)-2-(2-chloro-4-fluorophenyl)-1-oxo-2-azaspiro[4.5]dec-8-yl]-5-(pyrrolidin-1-yl)pyrazolo[1,5-a]pyrimidine-3-carboxamide;
N-[(trans)-2-(2-chloro-4-fluorophenyl)-1-oxo-2-azaspiro[4.5]dec-8-yl]-7-(trifluoromethyl)pyrazolo[1,5-a]pyrimidine-3-carboxamide;
N-[(trans)-2-(2-chloro-4-fluorophenyl)-1-oxo-2-azaspiro[4.5]dec-8-yl]-5-hydroxypyrazolo[1,5-a]pyrimidine-3-carboxamide;
N-[(trans)-2-(2-chloro-4-fluorophenyl)-1-oxo-2-azaspiro[4.5]dec-8-yl]-7-hydroxypyrazolo[1,5-a]pyrimidine-3-carboxamide;
N-[(trans)-2-(2-chloro-4-fluorophenyl)-1-oxo-2-azaspiro [4.5]dec-8-yl]-1H-pyrrolo[3,2-b]pyridine-3-carboxamide;
N-[(trans)-2-(2-chloro-4-fluorophenyl)-1-oxo-2-azaspiro[4.5]dec-8-yl]-6-(2-methoxyethyl)pyrazolo[1,5-a]pyrimidine-3-carboxamide;
N-[(trans)-1-oxo-2-phenyl-2-azaspiro[4.5]dec-8-yl]pyrazolo[1,5-a]pyrimidine-3-carboxamide;
5,7-dimethyl-N-[(trans)-1-oxo-2-phenyl-2-azaspiro [4.5]dec-8-yl]pyrazolo[1,5-a]pyrimidine-3-carboxamide;
N-[(trans)-1-oxo-2-phenyl-2-azaspiro[4.5]dec-8-yl]imidazo[1,2-b]pyridazine-3-carboxamide;
N-[(trans)-2-(2-chloro-4,6-difluorophenyl)-1-oxo-2-azaspiro[4.5]dec-8-yl]-7-(methoxymethyl)-5-methylpyrazolo[1,5-a]pyrimidine-3-carboxamide;
N-[(trans)-2-(2-chloro-4,6-difluorophenyl)-1-oxo-2-azaspiro[4.5]dec-8-yl]-5-(methoxymethyl)-7-methylpyrazolo[1,5-a]pyrimidine-3-carboxamide;
N-[(trans)-2-(2-chloro-4,6-difluorophenyl)-1-oxo-2-azaspiro[4.5]dec-8-yl]imidazo[1,2-b]pyridazine-3-carboxamide;
N-[(trans)-2-(2-chloro-4,6-difluorophenyl)-1-oxo-2-azaspiro[4.5]dec-8-yl]pyrazolo[1,5-a]pyrimidine-3-carboxamide;
N-[(trans)-2-(3,5-difluorophenyl)-1-oxo-2-azaspiro[4.5]dec-8-yl]pyrazolo[1,5-a]pyrimidine-3-carboxamide;
N-[(trans)-2-(4-chloropyridin-3-yl)-1-oxo-2-azaspiro[4.5]dec-8-yl]pyrazolo[1,5-a]pyrimidine-3-carboxamide;
N-[(trans)-2-(2-chloro-4-fluorophenyl)-1-oxo-2-azaspiro[4.5]dec-8-yl]-1-methyl-1H-indazole-3-carboxamide;
N-[(trans)-2-(2-chloro-4-fluorophenyl)-1-oxo-2-azaspiro [4.5]dec-8-yl]-4-iodo-1H-indazole-3-carboxamide;
N-{trans-4-[(2-chloro-4-fluorophenyl)carbamoyl]cyclohexyl}-1-methyl-1H-indazole-3-carboxamide;
N-{trans-4-[(2-chloro-4-fluorophenyl)carbamoyl]cyclohexyl}-2-methyl-2H-indazole-3-carboxamide;
N-[trans-2-(2-chloro-4-fluorophenyl)-1-oxo-2-azaspiro [4.5]dec-8-yl]-1H-indazole-3-carboxamide;
N-{trans-4-[(2-chloro-4-fluorophenyl)carbamoyl]cyclohexyl}-1H-indole-3-carboxamide;
N-{trans-4-[(2-chloro-4-fluorophenyl)carbamoyl]cyclohexyl}-1H-indazole-3-carboxamide;
N-{trans-4-[(2-chloro-4-fluorophenyl)carbamoyl]cyclohexyl}-5-fluoro-H-indazole-3-carboxamide;
2-chloro-N-{trans-4-[(2-chloro-4-fluorophenyl)carbamoyl]cyclohexyl}imidazo[1,2-a]pyridine-3-carboxamide;
N-[(trans)-2-(2-chlorophenyl)-1-oxo-2-azaspiro [4.5]dec-8-yl]pyrazolo[1,5-a]pyrimidine-3-carboxamide;
N-[(trans)-2-(2-chlorophenyl)-1-oxo-2-azaspiro[4.5]dec-8-yl]-5,7-dimethylpyrazolo[1,5-a]pyrimidine-3-carboxamide;
N⁵-[(trans)-2-(2-chlorophenyl)-1-oxo-2-azaspiro [4.5]dec-8-yl]-N⁴-ethyl-1H-imidazole-4,5-dicarboxamide;
N⁵-[(trans)-2-(2-chlorophenyl)-1-oxo-2-azaspiro [4.5]dec-8-yl]-N⁴-(2-methoxyethyl)-1H-imidazole-4,5-dicarboxamide;
N⁵-[(trans)-2-(2-chlorophenyl)-1-oxo-2-azaspiro[4.5]dec-8-yl]-N⁴-[2-(4-methylpiperidin-1-yl)ethyl]-1H-imidazole-4,5-dicarboxamide;
N⁵-[(trans)-2-(2-chlorophenyl)-1-oxo-2-azaspiro[4.5]dec-8-yl]-1H-imidazole-4,5-dicarboxamide;
N-[(trans)-2-(2-chlorophenyl)-1-oxo-2-azaspiro[4.5]dec-8-yl]-4-[(3-fluoroazetidin-1-yl)carbonyl]-1H-imidazole-5-carboxamide;
N-[(trans)-2-(2-chlorophenyl)-1-oxo-2-azaspiro [4.5]dec-8-yl]-4-(2-oxa-6-azaspiro [3.3]hept-6-ylcarbonyl)-1H-imidazole-5-carboxamide;
N⁵-[(trans)-2-(2-chlorophenyl)-1-oxo-2-azaspiro[4.5]dec-8-yl]-N⁴-methyl-1H-imidazole-4,5-dicarboxamide;
N-[(trans)-2-(2-chloro-4-fluoro-5-methylphenyl)-1-oxo-2-azaspiro [4.5]dec-8-yl]imidazo[1,2-b]pyridazine-3-carboxamide;
N-[(trans)-2-(2-chloro-4-fluoro-5-methylphenyl)-1-oxo-2-azaspiro[4.5]dec-8-yl]-5,7-dimethylpyrazolo[1,5-a]pyrimidine-3-carboxamide;
N-[(trans)-2-(2-chloro-4-fluoro-5-methylphenyl)-1-oxo-2-azaspiro [4.5]dec-8-yl]pyrazolo[1,5-a]pyrimidine-3-carboxamide;
N-[(trans)-2-(3-chlorophenyl)-1-oxo-2-azaspiro [4.5]dec-8-yl]pyrazolo[1,5-a]pyrimidine-3-carboxamide;
N-[(trans)-2-(3-chlorophenyl)-1-oxo-2-azaspiro [4.5]dec-8-yl]imidazo[1,2-b]pyridazine-3-carboxamide;
N-[(trans)-2-(3-chlorophenyl)-1-oxo-2-azaspiro[4.5]dec-8-yl]-5,7-dimethylpyrazolo[1,5-a]pyrimidine-3-carboxamide;
N-[(trans)-2-(2-chlorophenyl)-1-oxo-2-azaspiro [4.5]dec-8-yl]imidazo[1,2-b]pyridazine-3-carboxamide;
N-[(trans)-2-(2-chlorophenyl)-1-oxo-2-azaspiro [4.5]dec-8-yl]-1H-pyrazolo[4,3-b]pyridine-3-carboxamide;
N-[(trans)-2-(2-chlorophenyl)-1-oxo-2-azaspiro[4.5]dec-8-yl]-7-methylpyrazolo[1,5-a]pyrimidine-3-carboxamide;
N-[(trans)-2-(2-chlorophenyl)-1-oxo-2-azaspiro[4.5]dec-8-yl]-1H-pyrrolo[3,2-b]pyridine-3-carboxamide;
N-[(trans)-2-(3-chlorophenyl)-1-oxo-2-azaspiro[4.5]dec-8-yl]-1H-pyrrolo[3,2-b]pyridine-3-carboxamide;
N⁵-[(trans)-2-(3-chlorophenyl)-1-oxo-2-azaspiro[4.5]dec-8-yl]-N⁴-methyl-1H-imidazole-4,5-dicarboxamide;
N⁵-[(trans)-2-(3-chlorophenyl)-1-oxo-2-azaspiro[4.5]dec-8-yl]-N⁴-ethyl-1H-imidazole-4,5-dicarboxamide;
N⁵-[(trans)-2-(3-chlorophenyl)-1-oxo-2-azaspiro[4.5]dec-8-yl]-N⁴-(propan-2-yl)-1H-imidazole-4,5-dicarboxamide;
N⁵-[(trans)-2-(3-chlorophenyl)-1-oxo-2-azaspiro[4.5]dec-8-yl]-N⁴-cyclopropyl-1H-imidazole-4,5-dicarboxamide;
N-[(trans)-2-(3-chlorophenyl)-1-oxo-2-azaspiro[4.5]dec-8-yl]-4-[(3-fluoroazetidin-1-yl)carbonyl]-1H-imidazole-5-carboxamide;
N⁵-[(trans)-2-(3-chlorophenyl)-1-oxo-2-azaspiro [4.5]dec-8-yl]-N⁴-(2,2,2-trifluoroethyl)-1H-imidazole-4,5-dicarboxamide;
N⁵-[(trans)-2-(3-chlorophenyl)-1-oxo-2-azaspiro [4.5]dec-8-yl]-N⁴-(2-methoxyethyl)-1H-imidazole-4,5-dicarboxamide;
N-[(trans)-2-(3-chlorophenyl)-1-oxo-2-azaspiro[4.5]dec-8-yl]-4-[(4-methylpiperazin-1-yl)carbonyl]-1H-imidazole-5-carboxamide;
N⁵-[(trans)-2-(3-chlorophenyl)-1-oxo-2-azaspiro[4.5]dec-8-yl]-N⁴-(2-hydroxy-2-methylpropyl)-1H-imidazole-4,5-dicarboxamide;
N⁵-[(trans)-2-(3-chlorophenyl)-1-oxo-2-azaspiro[4.5]dec-8-yl]-N⁴-(1-methoxypropan-2-yl)-1H-imidazole-4,5-dicarboxamide;
N⁵-[(trans)-2-(3-chlorophenyl)-1-oxo-2-azaspiro[4.5]dec-8-yl]-N⁴-[(2S)-1-methoxypropan-2-yl]-1H-imidazole-4,5-dicarboxamide;
N⁵-[(trans)-2-(3-chlorophenyl)-1-oxo-2-azaspiro[4.5]dec-8-yl]-N⁴-[(2R)-1-methoxypropan-2-yl]-1H-imidazole-4,5-dicarboxamide;
N-[(trans)-2-(3-chlorophenyl)-1-oxo-2-azaspiro[4.5]dec-8-yl]-4-(morpholin-4-ylcarbonyl)-1H-imidazole-5-carboxamide;
N⁵-[(trans)-2-(3-chlorophenyl)-1-oxo-2-azaspiro[4.5]dec-8-yl]-N⁴-[2-(morpholin-4-yl)ethyl]-1H-imidazole-4,5-dicarboxamide;
N⁵-[(trans)-2-(2-chloro-4-fluoro-5-methylphenyl)-1-oxo-2-azaspiro[4.5]dec-8-yl]-N⁴-methyl-1H-imidazole-4,5-dicarboxamide;
N⁵-[(trans)-2-(2-chloro-4-fluoro-5-methylphenyl)-1-oxo-2-azaspiro[4.5]dec-8-yl]-N⁴-ethyl-1H-imidazole-4,5-dicarboxamide;
N⁵-[(trans)-2-(2-chloro-4-fluoro-5-methylphenyl)-1-oxo-2-azaspiro[4.5]dec-8-yl]-N⁴-[2-(4-methylpiperidin-1-yl)ethyl]-1H-imidazole-4,5-dicarboxamide;
N⁵-[(trans)-2-(2-chloro-4-fluoro-5-methylphenyl)-1-oxo-2-azaspiro[4.5]dec-8-yl]-1H-imidazole-4,5-dicarboxamide;
N-[(trans)-2-(2-chloro-4-fluoro-5-methylphenyl)-1-oxo-2-azaspiro[4.5]dec-8-yl]-4-[(3-fluoroazetidin-1-yl)carbonyl]-1H-imidazole-5-carboxamide;
N-[(trans)-2-(2-chloro-4-fluoro-5-methylphenyl)-1-oxo-2-azaspiro[4.5]dec-8-yl]-4-(2-oxa-6-azaspiro[3.3]hept-6-ylcarbonyl)-1H-imidazole-5-carboxamide;
N-[(trans)-2-(2-chloro-4-fluoro-5-methylphenyl)-1-oxo-2-azaspiro[4.5]dec-8-yl]-4-[(4-methylpiperazin-1-yl)carbonyl]-1H-imidazole-5-carboxamide;
N⁵-[(trans)-2-(2-chloro-4-fluoro-5-methylphenyl)-1-oxo-2-azaspiro[4.5]dec-8-yl]-N⁴-cyclopropyl-1H-imidazole-4,5-dicarboxamide;
N-[(trans)-2-(2-chloro-4-fluoro-5-methylphenyl)-1-oxo-2-azaspiro[4.5]dec-8-yl]-4-{[3-(difluoromethoxy)azetidin-1-yl]carbonyl}-1H-imidazole-5-carboxamide;
N⁵-[(trans)-2-(2-chloro-4-fluoro-5-methylphenyl)-1-oxo-2-azaspiro[4.5]dec-8-yl]-N⁴-(2,2,2-trifluoroethyl)-1H-imidazole-4,5-dicarboxamide;
N-[(trans)-2-(2-chloro-4,5-difluorophenyl)-1-oxo-2-azaspiro[4.5]dec-8-yl]imidazo[1,2-b]pyridazine-3-carboxamide;
N-[(trans)-2-(2-chloro-4,5-difluorophenyl)-1-oxo-2-azaspiro[4.5]dec-8-yl]-5,7-dimethylpyrazolo[1,5-a]pyrimidine-3-carboxamide;
N-[(trans)-2-(2-chloro-4-fluoro-5-methylphenyl)-1-oxo-2-azaspiro[5.5]undec-9-yl]pyrazolo[1,5-a]pyrimidine-3-carboxamide;
N-[(trans)-2-(2-chloro-4-fluoro-5-methylphenyl)-1-oxo-2-azaspiro[5.5]undec-9-yl]imidazo[1,2-b]pyridazine-3-carboxamide;
N-[(trans)-2-(2-chloro-4-fluoro-5-methylphenyl)-1-oxo-2-azaspiro[5.5]undec-9-yl]-5,7-dimethylpyrazolo[1,5-a]pyrimidine-3-carboxamide;
N-[(trans)-2-(2-chloro-4-fluoro-5-methylphenyl)-1-oxo-2-azaspiro[5.5]undec-9-yl]-4-[(3-fluoroazetidin-1-yl)carbonyl]-1H-imidazole-5-carboxamide;
N⁵-[(trans)-2-(2-chloro-4-fluoro-5-methylphenyl)-1-oxo-2-azaspiro[5.5]undec-9-yl]-N⁴-methyl-1H-imidazole-4,5-dicarboxamide;
N⁵-[(trans)-2-(2-chloro-4-fluoro-5-methylphenyl)-1-oxo-2-azaspiro[5.5]undec-9-yl]-N⁴-ethyl-1H-imidazole-4,5-dicarboxamide;
N⁵-[(trans)-2-(2-chloro-4-fluoro-5-methylphenyl)-1-oxo-2-azaspiro[5.5]undec-9-yl]-N⁴-(2-methoxyethyl)-1H-imidazole-4,5-dicarboxamide;
N⁴-[(trans)-2-(2-chloro-4-fluorophenyl)-3-hydroxy-1-oxo-2-azaspiro[4.5]dec-8-yl]-N⁵-methyl-1H-imidazole-4,5-dicarboxamide;
N⁴-[(3R,trans)-2-(2-chloro-4-fluorophenyl)-3-hydroxy-1-oxo-2-azaspiro[4.5]dec-8-yl]-N⁵-methyl-1H-imidazole-4,5-dicarboxamide;
N⁴-[(3S,trans)-2-(2-chloro-4-fluorophenyl)-3-hydroxy-1-oxo-2-azaspiro[4.5]dec-8-yl]-N⁵-methyl-1H-imidazole-4,5-dicarboxamide;
N⁵-[(trans)-2-(2-chloro-4-fluoro-5-methylphenyl)-1-oxo-2-azaspiro[5.5]undec-9-yl]-N⁴-cyclopropyl-1H-imidazole-4,5-dicarboxamide;
N⁵-[(trans)-2-(2-chloro-4-fluoro-5-methylphenyl)-1-oxo-2-azaspiro[5.5]undec-9-yl]-1H-imidazole-4,5-dicarboxamide;
N⁵-[(trans)-2-(2-chloro-4-fluoro-5-methylphenyl)-1-oxo-2-azaspiro[5.5]undec-9-yl]-N⁴-(propan-2-yl)-1H-imidazole-4,5-dicarboxamide;
N-[(trans)-2-(2-chloro-4-fluoro-5-methylphenyl)-1-oxo-2-azaspiro[5.5]undec-9-yl]-4-(2-oxa-6-azaspiro[3.3]hept-6-ylcarbonyl)-1H-imidazole-5-carboxamide;
N⁵-[(trans)-2-(2-chloro-4-fluoro-5-methylphenyl)-1-oxo-2-azaspiro[5.5]undec-9-yl]-N⁴-[2-(4-methylpiperidin-1-yl)ethyl]-1H-imidazole-4,5-dicarboxamide;
N-[(trans)-2-(2-chloro-4,5-difluorophenyl)-1-oxo-2-azaspiro[5.5]undec-9-yl]pyrazolo[1,5-a]pyrimidine-3-carboxamide;
N-[(trans)-2-(2-chloro-4,5-difluorophenyl)-1-oxo-2-azaspiro[5.5]undec-9-yl]imidazo[1,2-b]pyridazine-3-carboxamide;
N-[(trans)-2-(2-chloro-4,5-difluorophenyl)-1-oxo-2-azaspiro[5.5]undec-9-yl]-5,7-dimethylpyrazolo[1,5-a]pyrimidine-3-carboxamide;
N-[(trans)-2-(2-chloro-4,5-difluorophenyl)-1-oxo-2-azaspiro[5.5]undec-9-yl]-4-[(3-fluoroazetidin-1-yl)carbonyl]-1H-imidazole-5-carboxamide;
N⁵-[(trans)-2-(2-chloro-4,5-difluorophenyl)-1-oxo-2-azaspiro[5.5]undec-9-yl]-N⁴-methyl-1H-imidazole-4,5-dicarboxamide;
N⁵-[(trans)-2-(2-chloro-4,5-difluorophenyl)-1-oxo-2-azaspiro[5.5]undec-9-yl]-N⁴-ethyl-1H-imidazole-4,5-dicarboxamide;
6-bromo-N-[(trans)-2-(2-chloro-4-fluorophenyl)-1-oxo-2-azaspiro[4.5]dec-8-yl]-1-isopropyl-1H-indazole-3-carboxamide;
N⁵-[(trans)-2-(2-chloro-4,5-difluorophenyl)-1-oxo-2-azaspiro[5.5]undec-9-yl]-1H-imidazole-4,5-dicarboxamide;
N⁵-[(trans)-2-(2-chloro-4,5-difluorophenyl)-1-oxo-2-azaspiro[5.5]undec-9-yl]-N⁴-(propan-2-yl)-1H-imidazole-4,5-dicarboxamide;
N-[(trans)-2-(2-chloro-4,5-difluorophenyl)-1-oxo-2-azaspiro[5.5]undec-9-yl]-4-{[3-(difluoromethoxy)azetidin-1-yl]carbonyl}-1H-imidazole-5-carboxamide;
N-[(trans)-2-(2-chloro-4,5-difluorophenyl)-1-oxo-2-azaspiro[5.5]undec-9-yl]-4-(morpholin-4-ylcarbonyl)-1H-imidazole-5-carboxamide;
N⁵-[(trans)-2-(2-chloro-4,5-difluorophenyl)-1-oxo-2-azaspiro[5.5]undec-9-yl]-N⁴ cyclopropyl-1H-imidazole-4,5-dicarboxamide;
N-[(trans)-2-(2-chloro-4-fluorophenyl)-1-oxo-2-azaspiro[5.5]undec-9-yl]pyrazolo[1,5-a]pyrimidine-3-carboxamide;
N-[(trans)-2-(2-chloro-4-fluorophenyl)-1-oxo-2-azaspiro[5.5]undec-9-yl]imidazo[1,2-b]pyridazine-3-carboxamide;
N-[(trans)-2-(2-chloro-4-fluorophenyl)-1-oxo-2-azaspiro[5.5]undec-9-yl]-4-[(3-fluoroazetidin-1-yl)carbonyl]-1H-imidazole-5-carboxamide;
N⁵-[(trans)-2-(2-chloro-4-fluorophenyl)-1-oxo-2-azaspiro[5.5]undec-9-yl]-N⁴-cyclopropyl-1H-imidazole-4,5-dicarboxamide;
N⁵-[(trans)-2-(2-chloro-4-fluorophenyl)-1-oxo-2-azaspiro[5.5]undec-9-yl]-N⁴-(propan-2-yl)-1H-imidazole-4,5-dicarboxamide;
N-[(trans)-2-(2-chloro-4-fluorophenyl)-1-oxo-2-azaspiro[4.5]dec-8-yl]-5-(difluoromethyl)pyrazolo[1,5-a]pyrimidine-3-carboxamide;
N-[(trans)-2-(2-chloro-4-fluorophenyl)-1-oxo-2-azaspiro[4.5]dec-8-yl]-5,7-bis(difluoromethyl)pyrazolo[1,5-a]pyrimidine-3-carboxamide;
N-[(cis)-2-(2-chloro-4-fluorophenyl)-1-oxo-2-azaspiro[4.5]dec-8-yl]-1H-pyrrolo[3,2-b]pyridine-3-carboxamide;
N-[(cis)-2-(2-chloro-4-fluorophenyl)-1-oxo-2-azaspiro[4.5]dec-8-yl]-7-(difluoromethyl)-5-methylpyrazolo[1,5-a]pyrimidine-3-carboxamide;
N⁵-[(trans)-2-(2-chloro-4-fluoro-5-methylphenyl)-1-oxo-2-azaspiro[4.5]dec-8-yl]-N⁴-(2-methoxyethyl)-1H-imidazole-4,5-dicarboxamide;
N-[(cis)-2-(2-chloro-4-fluorophenyl)-1-oxo-2-azaspiro[4.5]dec-8-yl]-5-methylpyrazolo[1,5-a]pyrimidine-3-carboxamide;
N-[(cis)-2-(2-chloro-4-fluorophenyl)-1-oxo-2-azaspiro[4.5]dec-8-yl]-7-methylpyrazolo[1,5-a]pyrimidine-3-carboxamide;
N-[(cis)-2-(2-chloro-4-fluorophenyl)-1-oxo-2-azaspiro[4.5]dec-8-yl]-5-(methoxymethyl)-7-methylpyrazolo[1,5-a]pyrimidine-3-carboxamide;
N⁵-[(cis)-2-(2-chloro-4-fluorophenyl)-1-oxo-2-azaspiro[4.5]dec-8-yl]-N⁴-cyclopropyl-1H-imidazole-4,5-dicarboxamide;
N⁵-[(cis)-2-(2-chloro-4-fluorophenyl)-1-oxo-2-azaspiro[4.5]dec-8-yl]-N⁴-ethyl-1H-imidazole-4,5-dicarboxamide;
N-[(cis)-2-(2-chloro-4-fluorophenyl)-1-oxo-2-azaspiro[4.5]dec-8-yl]-4-[(3-fluoroazetidin-1-yl)carbonyl]-1H-imidazole-5-carboxamide;
N⁵-[(cis)-2-(2-chloro-4-fluorophenyl)-1-oxo-2-azaspiro[4.5]dec-8-yl]-N⁴-(2-methoxyethyl)-1H-imidazole-4,5-dicarboxamide;
N⁵-[(cis)-2-(2-chloro-4-fluorophenyl)-1-oxo-2-azaspiro[4.5]dec-8-yl]-1H-imidazole-4,5-dicarboxamide;
N-[(trans)-2-(2-chloro-4-fluorophenyl)-1-oxo-2-azaspiro[4.5]dec-8-yl]-1-methyl-1H-pyrrolo[3,2-b]pyridine-3-carboxamide;
N-[(trans)-2-(2-chloro-4-fluorophenyl)-1-oxo-2-azaspiro[4.5]dec-8-yl]-1-(2-hydroxyethyl)-1H-pyrrolo[3,2-b]pyridine-3-carboxamide;
N-[(trans)-2-(2-chloro-4-fluorophenyl)-1-oxo-2-azaspiro[4.5]dec-8-yl]-1-(2-methoxyethyl)-1H-pyrrolo[3,2-b]pyridine-3-carboxamide;
N-[(trans)-1-oxo-2-(pyridin-2-yl)-2-azaspiro[4.5]dec-8-yl]pyrazolo[1,5-a]pyrimidine-3-carboxamide;
N-[(trans)-2-(2-chloro-4-fluorophenyl)-1-oxo-2-azaspiro[4.5]dec-8-yl][1,2,4]triazolo[4,3-b]pyridazine-3-carboxamide;
N-[(trans)-2-(2-chloro-4-fluorophenyl)-1-oxo-2-azaspiro[4.5]dec-8-yl]-5,7-diethylpyrazolo[1,5-a]pyrimidine-3-carboxamide;
6-chloro-N-[(trans)-2-(2-chloro-4-fluorophenyl)-1-oxo-2-azaspiro[4.5]dec-8-yl]-8-[(3,3,3-trifluoropropyl)amino]imidazo[1,2-b]pyridazine-3-carboxamide;
N-[(trans)-2-(2-chloro-4-fluorophenyl)-1-oxo-2-azaspiro[4.5]dec-8-yl]-5H-pyrrolo[2,3-b]pyrazine-7-carboxamide;
methyl 3-({trans-4-[(2-chloro-4,6-difluorophenyl)carbamoyl]cyclohexyl}carbamoyl)-5-methylpyrazolo[1,5-a]pyrimidine-7-carboxylate;
methyl 3-({trans-4-[(2-chloro-4-fluorophenyl)carbamoyl]cyclohexyl}carbamoyl)-5-methylpyrazolo[1,5-a]pyrimidine-7-carboxylate;
N-[(trans)-2-(2-chloro-5-methoxyphenyl)-1-oxo-2-azaspiro [4.5]dec-8-yl]pyrazolo[1,5-a]pyrimidine-3-carboxamide;
N-{(trans)-2-[2-chloro-4-(trifluoromethoxy)phenyl]-1-oxo-2-azaspiro [4.5]dec-8-yl}pyrazolo[1,5-a]pyrimidine-3-carboxamide;
N-[(trans)-2-(2-chloro-4-fluorophenyl)-3-methyl-1-oxo-2-azaspiro[4.5]dec-8-yl]pyrazolo[1,5-a]pyrimidine-3-carboxamide (mixture of isomers at C3);
N-[(3S,trans)-2-(2-chloro-4-fluorophenyl)-3-methyl-1-oxo-2-azaspiro[4.5]dec-8-yl]pyrazolo[1,5-a]pyrimidine-3-carboxamide;
N-[(3R,trans)-2-(2-chloro-4-fluorophenyl)-3-methyl-1-oxo-2-azaspiro[4.5]dec-8-yl]pyrazolo[1,5-a]pyrimidine-3-carboxamide;
N-[(trans)-2-(2-chloro-4-fluorophenyl)-1-oxo-2-azaspiro[4.5]dec-8-yl]-6-[1-(dimethylamino)ethyl]-7-methylpyrazolo[1,5-a]pyrimidine-3-carboxamide;
N-[(trans)-2-(2-chloro-4-fluorophenyl)-1-oxo-2-azaspiro[4.5]dec-8-yl]-6-[(1R)-1-(dimethylamino)ethyl]-7-methylpyrazolo[1,5-a]pyrimidine-3-carboxamide;
N-[(trans)-2-(2-chloro-4-fluorophenyl)-1-oxo-2-azaspiro[4.5]dec-8-yl]-6-[(1S)-1-(dimethylamino)ethyl]-7-methylpyrazolo[1,5-a]pyrimidine-3-carboxamide;
N-{trans-4-[(2-chloro-4-fluorophenyl)carbamoyl]cyclohexyl}-6-(2-hydroxypropan-2-yl)-7-methylpyrazolo[1,5-a]pyrimidine-3-carboxamide;
N-{trans-4-[(2-chloro-4-fluorophenyl)carbamoyl]cyclohexyl}-6-hydroxyimidazo[1,2-b]pyridazine-3-carboxamide;
N-[(trans)-1-oxo-2-(thiophen-2-yl)-2-azaspiro[4.5]dec-8-yl]pyrazolo[1,5-a]pyrimidine-3-carboxamide;
methyl 3-chloro-4-{(trans)-1-oxo-8-[(pyrazolo[1,5-a]pyrimidin-3-ylcarbonyl)amino]-2-azaspiro[4.5]dec-2-yl}benzoate;
N-{trans-4-[(2-chloro-4,6-difluorophenyl)carbamoyl]cyclohexyl}-5-methyl-7,8-dihydro-6H-cyclopenta[e]pyrazolo[1,5-a]pyrimidine-3-carboxamide;
N-[(trans)-2-(2-chloro-4-fluorophenyl)-1-oxo-2-azaspiro[4.5]dec-8-yl]-7-hydroxy-5-methylpyrazolo[1,5-a]pyrimidine-3-carboxamide;
N-{4-[(2-chloro-4-fluorophenyl)carbamoyl]-4-fluorocyclohexyl}pyrazolo[1,5-a]pyrimidine-3-carboxamide (Single isomer); and
N⁴-{4-[(2-chloro-4-fluorophenyl)carbamoyl]-4-fluorocyclohexyl}-N⁵-methyl-1H-imidazole-4,5-dicarboxamide (Single isomer);
or a stereoisomer, a tautomer, an N-oxide, a hydrate, a solvate, or a salt thereof, or a mixture of same.

7: A method of preparing a compound of general formula (I) according to claim 1, said method comprising the step of allowing an intermediate compound of general formula (3-2): 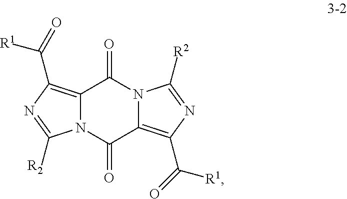
in which R¹ and R² are as defined for the compound of general formula (I) according to claim 1,
to react with a compound of general formula (C): 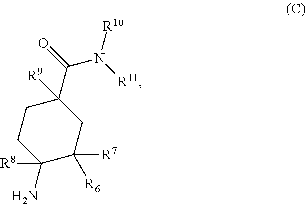
in which R⁶, R⁷, R⁸, R⁹, R¹⁰ and R¹¹ are as defined for the compound of general formula (I) according to claim 1,
thereby giving a compound of general formula (I): 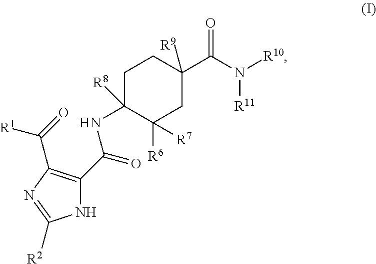
in which R¹, R², R⁶, R⁷, R⁸, R⁹, R¹⁰ and R¹¹ are as defined for the compound of general formula (I) according to claim 1.

8: A method of preparing a compound of general formula (I) according to claim 1, said method comprising the step of allowing a compound of general formula (I), a compound of formula (3-7): 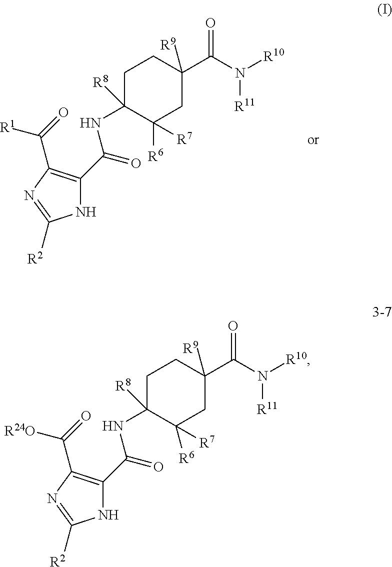
in which R¹ represents OR¹³, R²⁴ represents phenyl, and R², R⁶, R⁷, R⁸, R⁹, R¹⁰, R¹¹ and R¹³ are as defined for the compound of general formula (I) according to claim 1,
to react with an amine of formula HN(R¹⁴)R¹⁵,
wherein R¹⁴ and R¹⁵ are as defined for the compound of general formula (I) according to claim 1,
thereby giving a compound of general formula (I): 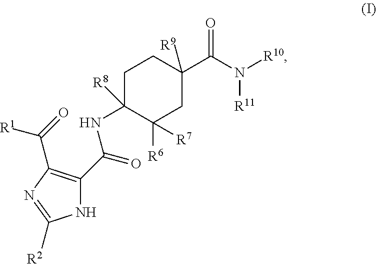
in which R¹ represents —N(R¹⁴)R¹⁵, and R², R⁶, R⁷, R⁸, R⁹, R¹⁰, R¹¹, R¹⁴ and R¹⁵ are as defined for the compound of general formula (I) according to claim 1.

9: A method of preparing a compound of general formula (I) according to claim 1, said method comprising the step of allowing an intermediate compound of general formula (3-3) or an intermediate of general formula (3-4): 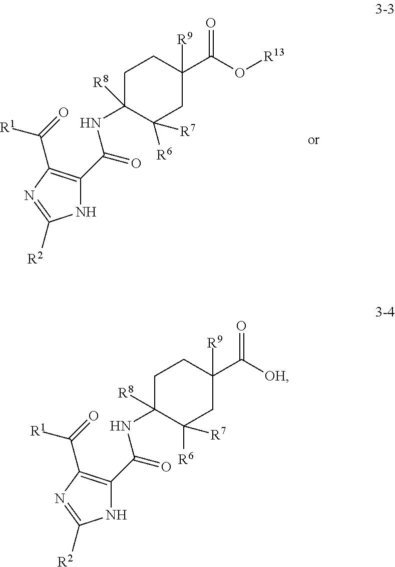
in which R¹, R², R⁶, R⁷, R⁸, R⁹, and R¹³ are as defined for the compound of general formula (I) according to claim 1,
to react with a compound of general formula (1-29): 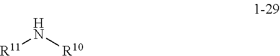
in which R¹⁰ and R¹¹ are as defined for the compound of general formula (I) according to claim 1,
thereby giving a compound of general formula (I): 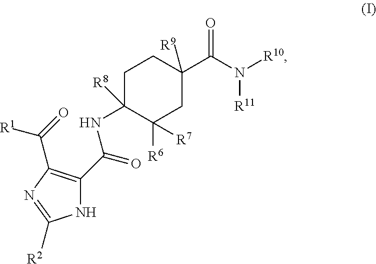
in which R¹, R², R⁶, R⁷, R⁸, R⁹, R¹⁰, and R¹¹ are as defined for the compound of general formula (I) according to claim 1.

10: A method of preparing a compound of general formula (I) according to claim 1, said method comprising the step of allowing an intermediate compound of general formula (3-8) or an intermediate of general formula (3-9): 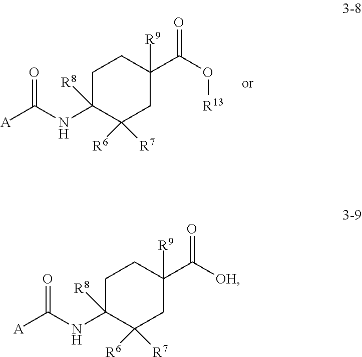
in which A represents A3, A4, A5, A6 A7, A8, A9, A10, A11, A12, A13, A14 or A15, and R⁶, R⁷, R⁸, R⁹, R¹³, A3, A4, A5, A6 A7, A8, A9, A10, A1, A12, A13, A14 and A15, are as defined for the compound of general formula (I) according to claim 1,
to react with a compound of general formula (1-29): 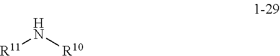
in which R¹⁰ and R¹¹ are as defined for the compound of general formula (I) according to claim 1,
thereby giving a compound of general formula (I): 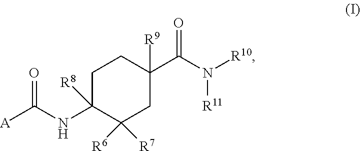
in which A represents A3, A4, A5 A6, A7, A8, A9, A10, A11, A12, A13, A14 or A15, and R⁶, R⁷, R⁸, R⁹, R¹⁰, R¹¹, A3, A4, A5, A6, A7, A8, A9, A10, A11, A12, A13, A14 and A15 are as defined for the compound of general formula (I) according to claim 1.

11: A method of preparing a compound of general formula (I) according to claim 1, said method comprising the step of allowing an intermediate compound of general formula (B): 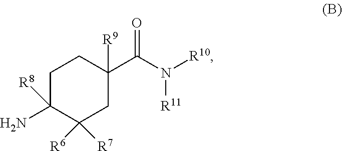
in which R⁶, R⁷, R⁸, R⁹, R¹⁰ and R¹¹ are as defined for the compound of general formula (I) according to claim 1,
to react with a compound of general formula (2-10): 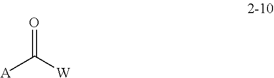
in which A represents A3, A4, A5, A6, A7, A8, A9, A10, A11, A12, A13, A14 or A15, and A3, A4, A5, A6, A7, A8, A9, A10, A11, A12, A13, A14 and A15 are as defined for the compound of general formula (I) according to claim 1, and W represents a hydroxyl group or a chloride,
thereby giving a compound of general formula (I): 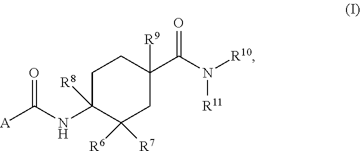
in which A represents A3, A4, A5, A6, A7, A8, A9, A10, A11, A12, A13, A14 or A15, and R⁶, R⁷, R⁸, R⁹, R¹⁰, R¹¹, A3, A4, A5, A6, A7, A8, A9, A10, A11, A12, A13, A14 and A15 are as defined for the compound of general formula (I) according to claim 1.

12: A method of preparing a compound of general formula (I) according to claim 1, said method comprising the step of allowing a compound of general formula (I) or a compound of formula (3-11): 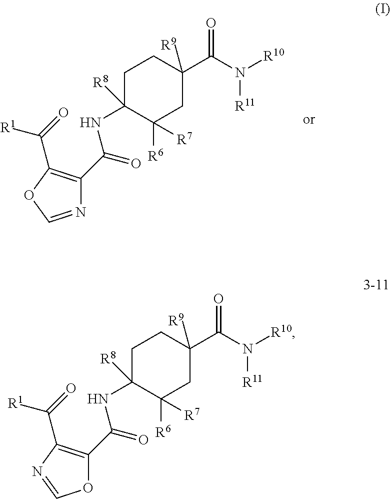
in which R¹ represents OR¹³, and R⁶, R⁷, R⁸, R⁹, R¹⁰, R¹¹ and R¹³ are as defined for the compound of general formula (I) according to claim 1,
to react with an amine of formula HN(R¹⁴)R¹⁵,
wherein R¹⁴ and R¹⁵ are as defined for the compound of general formula (I) according to claim 1,
thereby giving a compound of general formula (I): 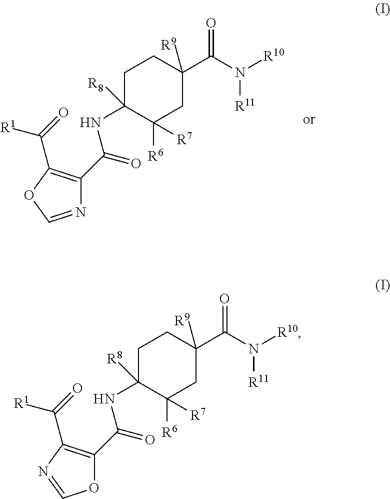
respectively, in which R¹ represents —N(R¹⁴)R¹⁵, and R⁶, R⁷, R⁸, R⁹, R¹⁰, R¹¹, R¹⁴ and R¹⁵ are as defined for the compound of general formula (I) according to claim 1.

13: A method for the treatment or prophylaxis of a disease, comprising administering to a patient in need thereof a compound of general formula (I), or a stereoisomer, a tautomer, an N-oxide, a hydrate, a solvate, or a salt thereof, or a mixture of same, according to claim 1.

14: A pharmaceutical composition comprising a compound of general formula (I), or a stereoisomer, a tautomer, an N-oxide, a hydrate, a solvate, or a salt thereof, or a mixture of same, according to claim 1, and a pharmaceutically acceptable diluent or carrier.

15: A pharmaceutical combination comprising:
one or more first active ingredients selected from a compound of general formula (I), or a stereoisomer, a tautomer, an N-oxide, a hydrate, a solvate, or a salt thereof, or a mixture of same, according to claim 1, and
one or more second active ingredients selected from chemotherapeutic anti-cancer agents.

16-17. (canceled)

18: The method of claim 13, wherein said disease is a disease of uncontrolled cell growth, proliferation and/or survival, an inappropriate cellular immune response, or an inappropriate cellular inflammatory response.

19: An intermediate compound or a salt thereof selected from: 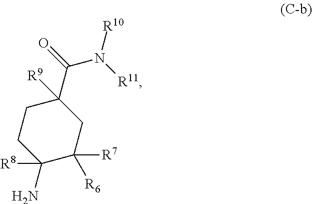
in which R⁶, R⁷, and R¹¹ are as defined for the compound of general formula (I) according to claim 1, and R⁸, R⁹ and R¹⁰ are as defined in embodiment b) for the compound of general formula (I) according to claim 1; 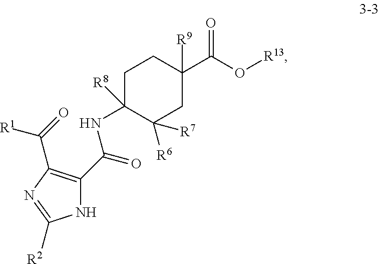
in which R¹, R², R⁶, R⁷, R⁸, R⁹, R¹³, R¹⁴ and R¹⁵ are as defined for the compound of general formula (I) according to claim 1; 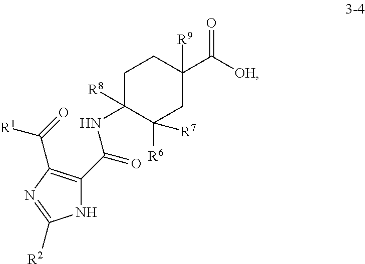
in which R¹, R², R⁶, R⁷, R⁸ and R⁹ are as defined for the compound of general formula (I) according to claim 1; 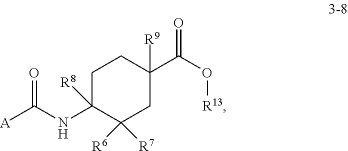
in which A represents A3, A4, A5, A6, A7, A8, A9, A10, A11, A12, A13, A14 or A15, and R⁶, R⁷, R⁸, R⁹, R¹³, A3, A4, A5, A6, A7, A8, A9, A10, A11, A12, A13, A14 and A15 are as defined for the compound of general formula (I) according to claim 1; 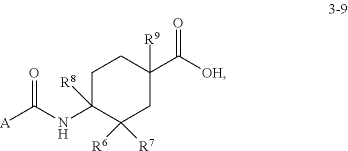
in which A represents A3, A4, A5, A6, A7, A8, A9, A10, A11, A12, A13, A14 or A15, and R⁶, R⁷, R⁸, R⁹, A3, A4, A5, A6, A7, A8, A9, A10, A11, A12, A13, A14 and A15 are as defined for the compound of general formula (I) according to claim 1; 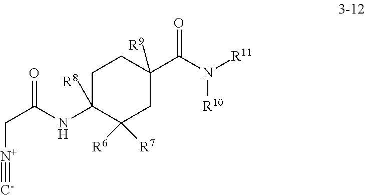
in which R⁶, R⁷, R⁸, R⁹, R¹⁰ and R¹¹ are as defined for the compound of general formula (I) according to claim 1; 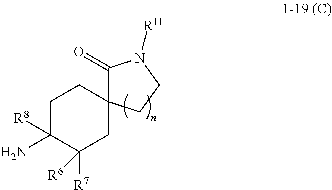
in which n is 1 or 2 and R⁶, R⁷, R⁸, and R¹¹ are as defined for the compound of general formula (I) according to claim 1; 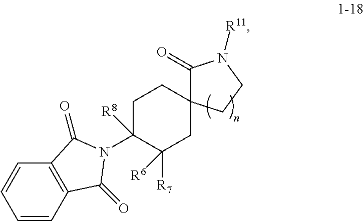
in which n is 1 or 2 and R⁶, R⁷, R⁸, and R¹¹ are as defined for the compound of general formula (I) according to claim 1; 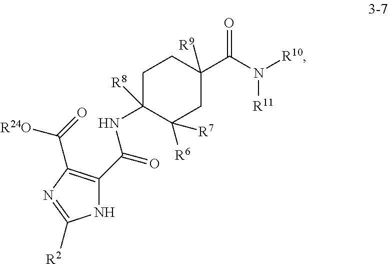
in which R²⁴ represents phenyl and R², R⁶, R⁷, R⁸, R⁹, R¹⁰, and R¹¹ are as defined for the compound of general formula (I) according to claim 1; and 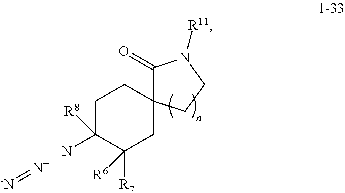
in which n is 1 or 2, and R⁶, R⁷, R⁸, and R¹¹ are as defined for the compound of general formula (I) according to claim 1.

20: An intermediate compound of formula (I-PG) or a salt thereof: 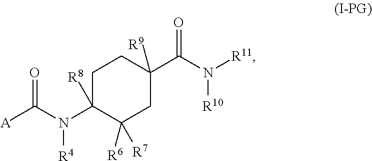
in which, A, X¹, R¹, R², R³, R⁴, R⁵, R⁶, R⁷, R⁸, R⁹, R¹⁰, R¹¹, R¹², R¹³, R¹⁶, R¹⁷, R²¹, and R²² are as defined for compound of formula (I) according to claim 1,
R¹⁴ is as defined for compound of formula (I) according to claim 1, or is an amine protecting group, such as a BOC group,
R¹⁵ is as defined for compound of formula (I) according to claim 1, or is an amine protecting group, such as a BOC group,
R¹⁸, is as defined for compound of formula (I) according to claim 1, or is an amine protecting group, such as a BOC group,
R¹⁹ is as defined for compound of formula (I) according to claim 1, or is an amine protecting group, such as a BOC group,
R²⁰ is as defined for compound of formula (I) according to claim 1, or is an amine protecting group, such as a BOC group,
wherein at least one of R¹⁴, R¹⁵, R¹⁸, R¹⁹, R²⁰ represents an amine protecting group, such as a BOC group.

21: A method for preparing a compound of formula (I) comprising using a compound according to claim 19.

22: The method of claim 18, wherein the disease of uncontrolled cell growth, proliferation and/or survival, inappropriate cellular immune response, or inappropriate cellular inflammatory response is a haematological tumour, a solid tumour and/or metastases thereof.

23: The method of claim 18, wherein the disease of uncontrolled cell growth, proliferation and/or survival, inappropriate cellular immune response, or inappropriate cellular inflammatory response is a leukaemia, myelodysplastic syndrome, a malignant lymphoma, a head and neck tumour, a tumour of the thorax, a gastrointestinal tumour, an endocrine tumour, a mammary tumour, a gynaecological tumour, a urological tumour, a skin tumour, or a sarcoma, and/or metastases thereof.

24: The method of claim 23, wherein the head and neck tumour is a brain tumour or a brain metastasis, or wherein the tumour of the thorax is a non-small cell lung tumour or a small cell lung tumour, or wherein the urological tumour is a renal tumour, a bladder tumour or a prostate tumour.

25: A method for preparing a compound of formula (I) comprising using a compound according to claim 20.


SEQUENCE LISTING (6 sequences)

SEQ ID NO: 1 - DNA, 20 bp, Homo sapiens
aagttcatccggcaccagtc

SEQ ID NO: 2 - DNA, 21 bp, Homo sapiens
tggcccttgaatcttctacga

SEQ ID NO: 3 - DNA, 22 bp, Homo sapiens
cccagaggcattgacaacaggg

SEQ ID NO: 4 - DNA, 20 bp, Homo sapiens
aggccagtgagttggttgtc

SEQ ID NO: 5 - DNA, 20 bp, Homo sapiens
agctctgagccttcagcatc

SEQ ID NO: 6 - DNA, 25 bp, Homo sapiens
tctgtggggaagaaattccataccg